(19) United States
(12) Patent Application Publication
(10) Pub. No.: US 2023/0091047 A1
(43) Pub. Date: Mar. 23, 2023

(54) FUSED RING PYRIMIDONE DERIVATIVES FOR USE IN THE TREATMENT OF HBV INFECTION OR OF HBV-INDUCED DISEASES

(71) Applicant: JANSSEN SCIENCES IRELAND UNLIMITED COMPANY, Co Cork (IE)

(72) Inventors: Sandrine Céline GROSSE, Turnhout (BE); Jan Martin BERKE, Willebroek (BE); Meng-yang HSIAO, Beerse (BE); Lili HU, Mechelen (BE); Edgar JACOBY, Vosselaar (BE); Tim Hugo Maria JONCKERS, Heist-op-den-Berg (BE); Bart Rudolf Romanie KESTELEYN, Berlare (BE); Stefaan Julien LAST, Beveren (BE); Carlolina MARTINEZ LAMENCA, Beerse (BE); Mathieu PERRIER, Beerse (BE); Serge Maria Aloysius PIETERS, Hulst (NL); Pierre Jean-Marie Bernard RABOISSON, Wavre (BE); Abdellah TAHRI, Sint-Pieters-Leeuw (BE); Koen VANDYCK, Paal-Beringer (BE); Wim Gaston VERSCHUEREN, Berchem (BE)

(21) Appl. No.: 17/438,767

(22) PCT Filed: Mar. 13, 2020
(86) PCT No.: PCT/EP2020/056884

§ 371 Date: Sep. 13, 2021

(30) Foreign Application Priority Data
Mar. 14, 2019 (EP) 19162954.2

(51) Int. Cl. C07D 471/04; C07D 519/00

(52) U.S. Cl. CPC C07D 471/04; C07D 519/00

(57) ABSTRACT

The present application relates to compounds according to Formula (I), pharmaceutical compositions comprising at least one of said compounds, their use as a medicament, and their use in treating chronic hepatitis B virus (HBV) infection. The disclosure further pertains to methods for preparing compounds according to Formula (I).

### FIELD OF THE INVENTION

This invention relates to fused ring pyrimidone derivatives, processes for their preparation, pharmaceutical compositions, and their use in treating chronic hepatitis B virus (HBV) infection.

### BACKGROUND OF THE INVENTION

Chronic hepatitis B virus (HBV) infection is a significant global health problem, affecting over 5% of the world population (over 350 million people worldwide and 1.25 million individuals in the U.S.).

Despite the availability of a prophylactic HBV vaccine, the burden of chronic HBV infection continues to be a significant unmet worldwide medical problem, due to suboptimal treatment options and sustained rates of new infections in most parts of the developing world.

Current treatments do not provide a cure and are limited to only two classes of agents (interferon alpha and nucleoside analogues/inhibitors of the viral polymerase); drug resistance, low efficacy, and tolerability issues limit their impact. The low cure rates of HBV are attributed at least in part to the fact that complete suppression of virus production is difficult to achieve with a single antiviral agent. However, persistent suppression of HBV DNA slows liver disease progression and helps to prevent hepatocellular carcinoma. Current therapy goals for HBV-infected patients are directed to reducing serum HBV DNA to low or undetectable levels, and to ultimately reducing or preventing the development of cirrhosis and hepatocellular carcinoma.

The HBV capsid protein plays essential functions during the viral life cycle. HBV capsid/core proteins form metastable viral particles or protein shells that protect the viral genome during intercellular passage, and also play a central role in viral replication processes, including genome encapsidation, genome replication, and virion morphogenesis and egress.

Capsid structures also respond to environmental cues to allow un-coating after viral entry.

Consistently, the appropriate timing of capsid assembly and dis-assembly, the appropriate capsid stability and the function of core protein have been found to be critical for viral infectivity.

Some fused pyrimidone derivatives are commercially available (CAS numbers: 2320260-88-4, 2109559-03-5, 2108279-21-4, 1793859-09-2, 1793086-51-7, 1792958-09-8, 1381725-74-1, 1381725-57-0, 1381687-98-4, 1381629-21-5, 1381629-07-7, 1381545-19-2, 1381545-12-5, 1381545-03-4, 1381541-85-0, 1381499-39-3, 1381495-33-5, 1381461-41-1, 1381461-33-1, 1381461-19-3, 1381435-78-4, 1381435-63-7, 1381435-59-1, 1381399-57-0, 1381336-67-9, 1381303-45-2, 1381303-34-9, 1381269-62-0, 1381269-55-1, 1381269-47-1, 1381269-40-4, 1381265-30-0, 1381265-10-6, 1381263-64-4, 1381263-57-5, 1381263-41-7, 1381263-34-8, 1380757-03-8, 1360422-55-4, 1360417-08-8, 1360409-93-3, 1360401-93-9, 1360388-01-7, 1360374-83-9, 1360367-17-4, 1360361-31-4, 1360359-43-8, 1360357-02-3, 1360354-25-1, 1360353-60-1, 1360318-16-6, 1360314-35-7, 1360308-53-7, 1360308-10-6, 1360302-54-0, 1360298-85-6, 1360298-49-2, 1360296-18-9, 1360292-31-4, 1360287-16-6, 1360265-66-2, 1360262-15-2, 1360247-61-5, 1360224-96-9, 1360221-74-4, 1360217-36-2, 1360074-93-6, 1351251-16-5, 1351125-54-6, 1351116-83-0, 1351084-08-6, 1351083-58-3, 1351068-72-8, 1351051-15-4, 1351050-03-7, 1351029-40-7, 1351022-92-8, 1351010-57-5, 1351010-32-6, 1351008-30-4, 1350996-64-3, 1350995-80-0, 1334244-38-0, 1214501-68-4, 1214426-23-9, 1177959-41-9, 1177951-84-6, 1177876-75-3, 1177873-76-5, 1177735-52-2, 1177669-85-0, 1177628-48-6, 1177612-26-8, 1177607-23-6, 1177589-03-5, 1177524-50-3, 1177468-49-3, 1177460-83-1, 1177431-65-0, 1177363-71-1, 1177152-98-5, 1029643-54-6, 958608-09-8.

WO2008/130581 discloses fused pyrimidinone derivatives having GPR119 modulatory activity; ACS Med Chem Lett 2017, 8, 1258-1263 discloses a fused pyrimidinone derivative having ULK1 inhibitory activity; WO2010/111880 discloses fused pyrimidinone derivatives as inhibitors of the nuclear export of GSK3; EP2078719 discloses bicyclic pyrimidine derivatives having MGAT inhibitory activity; and Mini-Reviews Med Chem, 2013, 13, 749-776 provides a review of small molecule inhibitors of HBV.

There is a need in the art for therapeutic agents that can increase the suppression of virus production and that can treat, ameliorate, or prevent HBV infection. Administration of such therapeutic agents to an HBV infected patient, either as monotherapy or in combination with other HBV treatments or ancillary treatments, will lead to significantly reduced virus burden, improved prognosis, diminished progression of the disease and enhanced seroconversion rates.

Particularly, it is desired to find compounds that are capable of capsid assembly modulation.

### SUMMARY OF THE INVENTION

The present invention relates to compounds that are capable of capsid assembly modulation. The compounds of the present invention may provide a beneficial balance of properties with respect to prior art compounds. In particular, they may display favourable metabolic properties, tissue distribution, safety and pharmaceutical profile. Thus, provided herein is a compound of Formula (I)

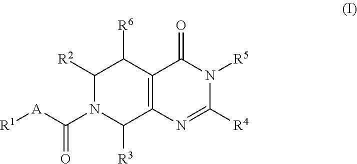

including any of its stereoisomers or tautomeric forms thereof, wherein:
A is a bond or NH;
R¹ is a 5- to 10-membered monocyclic or bicyclic ring system, more particularly a 5 to 9-membered monocyclic or bicyclic ring, wherein the 5- to 10-membered monocyclic or bicyclic ring system, more particularly the 5- to 9-membered monocyclic or bicyclic ring system, optionally contains 1 to 3 heteroatoms, the heteroatoms each independently being selected from N, O and S;
wherein the 5- to 10-membered monocyclic or bicyclic ring, more particularly the 5- to 9-membered monocyclic or bicyclic ring is optionally substituted with one or more substituents each independently selected from the group consisting of halo, CN, CF₃, CF₂H, CFH₂, CF₂CH₃, C₁₋₆alkyl, OC₁₋₆alkyl, OCF₃, OCF₂H, and C₃₋₄cycloalkyl; or R¹ is selected from the group consisting of 1-methyl-2-oxo-1,3-dihydro-1H-benzo[d]imidazol-5-yl, 1-oxo-isoindolin-5-yl, and 1,1-dioxo-benzo[b]thiophen-5-yl;
R² is selected from the group consisting of hydrogen, C₁₋₆alkyl, CF₃, CHF₂, CH₂F, phenyl and fluorophenyl;
R³ is hydrogen;

### R⁴ is X—R′;

wherein X is NR″, S or O;
wherein R′ is hydrogen, C₁₋₄alkyl, C₁₋₆alkyl substituted with OH, or C₂₋₃alkenyl, when X is NR″;
wherein R′ is C₁₋₆alkyl, when X is S;
wherein R′ is C₁₋₆alkyl, when X is O;
wherein R″ is selected from the group consisting of hydrogen, Cycle1, Aryl1, C₂₋₄alkynyl, C₁₋₆alkyl and C₁₋₆alkyl substituted with one or more substituents each independently selected from the group consisting of
fluoro,
OH,
COO₂R¹⁶,
OCONHR¹⁷,
C₃₋₆cycloalkyl,
C₃₋₆cycloalkyl substituted with one or more from among C₁₋₆alkyl,
N-acetyl piperidine,
cubanyl,
benzo[d][1,3]dioxole, and
Aryl2;
   wherein R¹⁶ is hydrogen or C₁₋₆alkyl;
   wherein R¹⁷ is C₁₋₆alkyl;
   wherein Cycle1 is selected from the group consisting of
C₃₋₈cycloalkyl,
C₃₋₈cycloalkyl containing a heteroatom, said heteroatom being an oxygen atom,
C₃₋₈cycloalkyl substituted with one or more substituents each independently selected from CH₃ and Aryl2,
C₃₋₈cycloalkyl containing a heteroatom and being substituted with one or more substituents each independently selected from the group consisting of CH₃, cyclopropyl, and Aryl2, said heteroatom being an oxygen atom,
a 5- to 9-membered fused bicyclic unsaturated or saturated ring system, in particular a saturated heterocycle fused with an aromatic ring, which may be optionally substituted with OCH₃,
a 5- to 9-membered bridged bicyclic unsaturated or saturated ring system optionally substituted with 1, 2 or 3 CH₃ substituents,
a C₅₋₁₂spirocycloalkyl, and
cubanyl;
   wherein Aryl1 is selected from the group consisting of phenyl, monocyclic heteroaryl, and bicyclic heteroaryl which is in particular an aromatic ring fused to a saturated ring or an aromatic ring fused to another aromatic ring, said Aryl1 being optionally substituted with CH₃;
   wherein Aryl2 is selected from the group consisting of phenyl, monocyclic heteroaryl, and bicyclic heteroaryl, said Aryl2 being optionally substituted with one or more substituents each independently selected from the group consisting of halogens, CF₃, CF₂H, CH₂F, C₁₋₄alkyl, C₃₋₆cycloalkyl, CN, CONR¹⁸R¹⁹, OH, OCF₃, OCF₂H, OCH₂F, OC₁₋₄alkyl, OC₃₋₆cycloalkyl, SO₂CH₃, imidazolyl optionally substituted with CH₃, phenyl optionally substituted with fluoro, and triazolyl;
   wherein R¹⁸ and R¹⁹ are independently selected from the group consisting of hydrogen, C₁₋₆alkyl and C₃₋₆cycloalkyl;
   or wherein N, R′ and R″ together form a cycle or cycle system selected from the group consisting of
a C₃₋₈cycloalkyl ring,
a C₃₋₈cycloalkyl ring containing a heteroatom, said heteroatom being an oxygen atom, and optionally being substituted with CH₃,
a C₃₋₈cycloalkyl ring substituted with one or more substituents each independently selected from the group consisting of C₁₋₆alkyl, phenyl, C₂₋₆alkynyl and C₃₋₆cycloalkyl,
a C₃₋₈cycloalkyl ring containing a heteroatom and being substituted with one or more substituents each independently selected from the group consisting of C₁₋₆alkyl, CN, phenyl, C₂₋₆alkynyl and C₃₋₆cycloalkyl, said heteroatom being an oxygen atom,
a C₅₋₁₂-spirocycloalkyl optionally substituted with CH₃, and
a C₅₋₆ bridged bicyclic saturated ring system;
   R⁵ is selected from the group consisting of hydrogen, C₁₋₆alkyl, C₂₋₃alkenyl, Cycle2 and Aryl3;
   wherein C₁₋₆alkyl is optionally substituted with one or more substituents each independently selected from the group consisting of phenyl, methoxyphenyl, OC₁₋₆alkyl, NHSO₂CH₃, C₃₋₆cycloalkyl, and C₃₋₆cycloalkyl containing a heteroatom, said heteroatom being an oxygen atom;
   wherein Cycle2 is selected from the group consisting of
C₃₋₆cycloalkyl,
C₃₋₆cycloalkyl containing SO₂ or a heteroatom, the heteroatom being selected from the group consisting of oxygen and nitrogen,
C₃₋₆cycloalkyl substituted with CO₂R^{20a}, CONHR^{20b} or SO₂C₁₋₆alkyl,
C₃₋₆cycloalkyl containing SO₂ or a heteroatom and being substituted with CO₂R^{20a}, CONHR^{20b} or SO₂C₁₋₆alkyl, the heteroatom being selected from the group consisting of oxygen and nitrogen,
a 5-membered bridged bicyclic saturated ring substituted with CO₂C₁₋₆alkyl or CONHR^{20b},
cubanyl optionally substituted with CO₂C₁₋₆alkyl or CONHR^{20b},
isoindoline-1-one, and
indoline-2-one;
   wherein R^{20a} is hydrogen or C₁₋₆alkyl;
   wherein R^{20b} is C₁₋₆alkyl or C₃₋₆cycloalkyl;
   wherein Aryl3 is selected from the group consisting of phenyl, monocyclic heteroaryl, and bicyclic heteroaryl, said Aryl3 being optionally substituted with one or more substituents each independently selected from the group consisting of halogen, C₁₋₆alkyl, CF₃, CF₂H, CH₂F, CN, OC₁₋₆alkyl, OCF₃, OCF₂H, OCH₂F, OC₃₋₆cycloalkyl, SO₂R²¹, SO₂NHR²², CO₂R²³, COR²⁴, CONR²⁵R²⁶, NHR²⁷, NHCOR²⁸, Cycle3 and Aryl4;
   wherein R²¹ is C₁₋₆alkyl or C₃₋₆cycloalkyl;
   wherein R²² is C₁₋₆alkyl or pyridine;
   wherein R²³ is hydrogen or C₁₋₆alkyl;
   wherein R²⁴ is selected from the group consisting of C₁₋₆alkyl, C₅₋₆heterocycle and C₅₋₆heterocycle substituted with CH₃;
   wherein R²⁵ is hydrogen or CH₃;
   wherein R²⁶ is selected from the group consisting of hydrogen,
C₁₋₆alkyl,
C₁₋₆alkyl optionally substituted with one or more substituents each independently selected from the group consisting of OH, OCH₃, NH₂, CO₂H, C₃₋₆heterocycloalkyl and C₃₋₆heterocycloalkyl substituted with CH₃,
C₃₋₆cycloalkyl;
C₃₋₆cycloalkyl containing a heteroatom, said heteroatom being an oxygen atom;
C₃₋₆cycloalkyl substituted with CO₂H; and
C₃₋₆cycloalkyl containing a heteroatom and being substituted with CO₂H, said heteroatom being an oxygen atom;
   wherein R²⁷ is selected from the group consisting of C₁₋₆alkyl,
C₁₋₆alkyl substituted with C₃₋₆heterocycloalkyl, and
C₃₋₆heterocycloalkyl;
   wherein R²⁸ is C₁₋₆alkyl or C₃₋₆cycloalkyl;
   wherein Cycle3 is selected from the group consisting of
C₃₋₆cycloalkyl,
C₃₋₆heterocycloalkyl,
C₃₋₆heterocycloalkyl substituted with one or more substituents each independently selected from the group consisting of OH, CH₂OH, CO₂R²⁹, NHCH₃ or NHCO₂t-Bu; and imidazolidin-4-one substituted with CH₃;
   wherein R²⁹ is hydrogen or C₁₋₆alkyl;
   wherein Aryl4 is selected from the group consisting of monocyclic heteroaryl and bicyclic heteroaryl, said monocyclic or bicyclic heteroaryl being optionally substituted with one or two substituents each independently selected from the group consisting of halogens, CF₃, CH₂F, C₁₋₆alkyl, C₃₋₆cycloalkyl, OCF₃, OCH₂F, OC₁₋₆alkyl, OC₃₋₆cycloalkyl, CO₂R³⁰, SO₂CH₃, and morpholine;
   wherein R³⁰ is hydrogen or C₁₋₆alkyl;
   wherein R′, R″ and R⁵ are not all hydrogen; and
   wherein R⁶ is hydrogen, CH₃, CF₃ or CF₂H;
   or a pharmaceutically acceptable salt thereof,
   for use in the prevention or treatment of an HBV infection or of an HBV-induced disease.

In an additional aspect, provided herein is a method of treating or preventing HBV infection or an HBV-induced disease in a subject in need thereof, said method comprising administering to said subject a therapeutically effective amount of a compound of Formula (I)

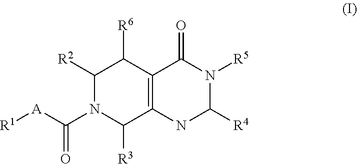

including any of its stereoisomers or tautomeric forms thereof, wherein:
A is a bond or NH;
R¹ is a 5- to 10-membered monocyclic or bicyclic ring system, more particularly a 5- to 9-membered monocyclic or bicyclic ring system, wherein the 5- to 10-membered monocyclic or bicyclic ring system, more particularly the 5- to 9-membered monocyclic or bicyclic ring system, optionally contains 1 to 3 heteroatoms, the heteroatoms each independently being selected from N, O and S;
wherein the 5- to 10-membered monocyclic or bicyclic ring system, more particularly the 5- to 9-membered monocyclic or bicyclic ring system is optionally substituted with one or more substituents each independently selected from the group consisting of halo, CN, CF₃, CF₂H, CFH₂, CF₂CH₃, C₁₋₆alkyl, OC₁₋₆alkyl, OCF₃, OCF₂H, and C₃₋₄cycloalkyl;
or R¹ is selected from the group consisting of 1-methyl-2-oxo-1,3-dihydro-1H-benzo[d]imidazol-5-yl, 1-oxo-isoindolin-5-yl, and 1,1-dioxo-benzo[b]thiophen-5-yl;
R² is selected from the group consisting of hydrogen, C₁₋₆alkyl, CF₃, CHF₂, CH₂F, phenyl and fluorophenyl;
R³ is hydrogen;

### R⁴ is X—R′;

wherein X is NR″, S or O;
wherein R′ is hydrogen, C₁₋₄alkyl, C₁₋₆alkyl substituted with OH, or C₂₋₃alkenyl, when X is NR″;
wherein R′ is C₁₋₆alkyl, when X is S;
wherein R′ is C₁₋₆alkyl, when X is O;
wherein R″ is selected from the group consisting of hydrogen, Cycle1, Aryl1, C₂₋₄alkynyl, C₁₋₆alkyl and C₁₋₆alkyl substituted with one or more substituents each independently selected from the group consisting of
fluoro,
OH,
CO₂R¹⁶,
OCONHR¹⁷,
C₃₋₆cycloalkyl,
C₃₋₆cycloalkyl substituted with one or more from among C₁₋₆alkyl,
N-acetyl piperidine,
cubanyl,
benzo[d][1,3]dioxole, and
Aryl2;
   wherein R¹⁶ is hydrogen or C₁₋₆alkyl;
   wherein R¹⁷ is C₁₋₆alkyl;
   wherein Cycle1 is selected from the group consisting of
C₃₋₈cycloalkyl,
C₃₋₈cycloalkyl containing a heteroatom, said heteroatom being an oxygen atom,
C₃₋₈cycloalkyl substituted with one or more substituents each independently selected from CH₃ and Aryl2,
C₃₋₈cycloalkyl containing a heteroatom and being substituted with one or more substituents each independently selected from the group consisting of CH₃, cyclopropyl, and Aryl2, said heteroatom being an oxygen atom,
a 5- to 9-membered fused bicyclic unsaturated or saturated ring system, in particular a saturated heterocycle fused with an aromatic ring which may be optionally substituted with OCH₃,
a 5- to 9-membered bridged bicyclic unsaturated or saturated ring system optionally substituted with 1, 2 or 3 CH₃ substituents,
a C₅₋₁₂spirocycloalkyl, and
cubanyl;
   wherein Aryl1 is selected from the group consisting of phenyl, monocyclic heteroaryl, and bicyclic heteroaryl which is in particular an aromatic ring fused to a saturated ring or an aromatic ring fused to another aromatic ring, said Aryl1 being optionally substituted with CH₃;
   wherein Aryl2 is selected from the group consisting of phenyl, monocyclic heteroaryl, and bicyclic heteroaryl, said Aryl2 being optionally substituted with one or more substituents each independently selected from the group consisting of halogens, CF₃, CF₂H, CH₂F, C₁₋₄alkyl, C₃₋₆cycloalkyl, CN, CONR¹⁸R¹⁹, OH, OCF₃, OCF₂H, OCH₂F, OC₁₋₄alkyl, OC₃₋₆cycloalkyl, SO₂CH₃, imidazolyl optionally substituted with CH₃, phenyl optionally substituted with fluoro, and triazolyl;
   wherein R¹⁸ and R¹⁹ are independently selected from the group consisting of hydrogen, C₁₋₆alkyl and C₃₋₆cycloalkyl;
   or wherein N, R′ and R″ together form a cycle or cycle system selected from the group consisting of
a C₃₋₈cycloalkyl ring,
a C₃₋₈cycloalkyl ring containing a heteroatom, said heteroatom being an oxygen atom, and optionally being substituted with CH₃,
a C₃₋₈cycloalkyl ring substituted with one or more substituents each independently selected from the group consisting of C₁₋₆alkyl, phenyl, C₂₋₆alkynyl and C₃₋₆cycloalkyl,
a C₃₋₈cycloalkyl ring containing a heteroatom and being substituted with one or more substituents each independently selected from the group consisting of C₁₋₆alkyl, CN, phenyl, C₂₋₆alkynyl and C₃₋₆cycloalkyl, said heteroatom being an oxygen atom,
a C₅₋₁₂-spirocycloalkyl optionally substituted with CH₃, and
a C₅₋₆ bridged bicyclic saturated ring system;
   R⁵ is selected from the group consisting of hydrogen, C₁₋₆alkyl, C₂₋₃alkenyl, Cycle2 and Aryl3;
   wherein C₁₋₆alkyl is optionally substituted with one or more substituents each independently selected from the group consisting of phenyl, methoxyphenyl, OC₁₋₆alkyl, NHSO₂CH₃, C₃₋₆cycloalkyl, and C₃₋₆cycloalkyl containing a heteroatom, said heteroatom being an oxygen atom;
   wherein Cycle2 is selected from the group consisting of
C₃₋₆cycloalkyl,
C₃₋₆cycloalkyl containing SO₂ or a heteroatom, the heteroatom being selected from the group consisting of oxygen and nitrogen,
C₃₋₆cycloalkyl substituted with CO₂R^{20a}, CONHR^{20b} or SO₂C₁₋₆alkyl, C₃₋₆cycloalkyl containing SO₂ or a heteroatom and being substituted with CO₂R^{20a}, CONHR^{20b} or SO₂C₁₋₆alkyl, the heteroatom being selected from the group consisting of oxygen and nitrogen,
a 5-membered bridged bicyclic saturated ring substituted with CO₂C₁₋₆alkyl or CONHR^{20b},
cubanyl optionally substituted with CO₂C₁₋₆alkyl or CONHR^{20b},
isoindoline-1-one, and
indoline-2-one:
   wherein R^{20a} is hydrogen or C₁₋₆alkyl;
   wherein R^{20b} is C₁₋₆alkyl or C₃₋₆cycloalkyl;
   wherein Aryl3 is selected from the group consisting of phenyl, monocyclic heteroaryl, and bicyclic heteroaryl, said Aryl3 being optionally substituted with one or more substituents each independently selected from the group consisting of halogen, C₁₋₆alkyl, CF₃, CF₂H, CH₂F, CN, OC₁₋₆alkyl, OCF₃, OCF₂H, OCH₂F, OC₃₋₆cycloalkyl, SO₂R²¹, SO₂NHR²², CO₂R²³, COR²⁴, CONR²⁵R²⁶, NHR²⁷, NHCOR²⁸, Cycle3 and Aryl4;
   wherein R²¹ is C₁₋₆alkyl or C₃₋₆cycloalkyl;
   wherein R²² is C₁₋₆alkyl or pyridine;
   wherein R²³ is hydrogen or C₁₋₆alkyl;
   wherein R²⁴ is selected from the group consisting of C₁₋₆alkyl, C₅₋₆heterocycle in particular C₅₋₆heterocycloalkyl and C₅₋₆heterocycle, in particular C₅₋₆heterocycloalkyl, substituted with CH₃;
   wherein R²⁵ is hydrogen or CH₃;
   wherein R²⁶ is selected from the group consisting of
hydrogen,
C₁₋₆alkyl,
C₁₋₆alkyl optionally substituted with one or more substituents each independently selected from the group consisting of OH, OCH₃, NH₂, CO₂H, C₃₋₆heterocycloalkyl and C₃₋₆heterocycloalkyl substituted with CH₃, C₃₋₆cycloalkyl;
C₃₋₆cycloalkyl containing a heteroatom, said heteroatom being an oxygen atom;
C₃₋₆cycloalkyl substituted with CO₂H; and
C₃₋₆cycloalkyl containing a heteroatom and being substituted with CO₂H, said heteroatom being an oxygen atom;
   wherein R²⁷ is selected from the group consisting of
C₁₋₆alkyl,
C₁₋₆alkyl substituted with C₃₋₆heterocycloalkyl, and
C₃₋₆heterocycloalkyl;
   wherein R²⁸ is C₁₋₆alkyl or C₃₋₆cycloalkyl;
   wherein Cycle3 is selected from the group consisting of
C₃₋₆cycloalkyl,
C₃₋₆heterocycloalkyl,
C₃₋₆heterocycloalkyl substituted with one or more substituents each independently selected from the group consisting of OH, CH₂OH, CO₂R²⁹, NHCH₃ or NHCO₂t-Bu; and
imidazolidin-4-one substituted with CH₃;
   wherein R²⁹ is hydrogen or C₁₋₆alkyl;
   wherein Aryl4 is selected from the group consisting of monocyclic heteroaryl and bicyclic heteroaryl, said monocyclic or bicyclic heteroaryl being optionally substituted with one or two substituents each independently selected from the group consisting of halogens, CF₃, CH₂F, C₁₋₆alkyl, C₃₋₆cycloalkyl, OCF₃, OCH₂F, OC₁₋₆alkyl, OC₃₋₆cycloalkyl, CO₂R³⁰, SO₂CH₃, and morpholine;
   wherein R³⁰ is hydrogen or C₁₋₆alkyl;
   wherein R′, R″ and R⁵ are not all hydrogen; and
   wherein R⁶ is hydrogen, CH₃, CF₃ or CF₂H;
   or a pharmaceutically acceptable salt thereof.

Also provided herein is a compound of Formula (I)

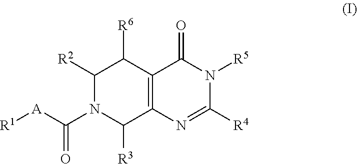

including any of its stereoisomers or tautomeric forms thereof, wherein:
A is a bond or NH;
R¹ is a 5- to 10-membered monocyclic or bicyclic ring system, more particularly a 5- to 9-membered monocyclic or bicyclic ring system, wherein the 5- to 10-membered monocyclic or bicyclic ring system, more particularly the 5- to 9-membered monocyclic or bicyclic ring system, optionally contains 1 to 3 heteroatoms, the heteroatoms each independently being selected from N, O and S;
wherein the 5- to 10-membered monocyclic or bicyclic ring, more particularly the 5- to 9-membered monocyclic or bicyclic ring is optionally substituted with one or more substituents each independently selected from the group consisting of halo, CN, CF₃, CF₂H, CFH₂, CF₂CH₃, C₁₋₆alkyl, OC₁₋₆alkyl, OCF₃, OCF₂H, and C₃₋₄cycloalkyl;
or R¹ is selected from the group consisting of 1-methyl-2-oxo-1,3-dihydro-1H-benzo[d]imidazol-5-yl, 1-oxo-isoindolin-5-yl, and 1,1-dioxo-benzo[b]thiophen-5-yl;
R² is selected from the group consisting of hydrogen, C₁₋₆alkyl, CF₃, CHF₂, CH₂F, phenyl and fluorophenyl;
R³ is hydrogen;

### R⁴ is X—R′;

wherein X is NR″, S or O;
wherein R′ is hydrogen, C₁₋₄alkyl, C₁₋₆alkyl substituted with OH, or C₂₋₃alkenyl, when X is NR″;
wherein R′ is C₁₋₆alkyl, when X is S;
wherein R′ is C₁₋₆alkyl, when X is O;
wherein R″ is selected from the group consisting of hydrogen, Cycle1, Aryl1, C₂₋₄alkynyl, C₁₋₆alkyl and C₁₋₆alkyl substituted with one or more substituents each independently selected from the group consisting of
fluoro,
OH,
CO₂R¹⁶,
OCONHR¹⁷,
C₃₋₆cycloalkyl,
C₃₋₆cycloalkyl substituted with one or more from among C₁₋₆alkyl,
N-acetyl piperidine,
cubanyl,
benzo[d][1,3]dioxole, and
Aryl2;
   wherein R¹⁶ is hydrogen or C₁₋₆alkyl;
   wherein R¹⁷ is C₁₋₆alkyl;
   wherein Cycle1 is selected from the group consisting of
C₃₋₈cycloalkyl,
C₃₋₈cycloalkyl containing a heteroatom, said heteroatom being an oxygen atom,
C₃₋₈cycloalkyl substituted with one or more substituents each independently selected from CH₃ and Aryl2,
C₃₋₈cycloalkyl containing a heteroatom and being substituted with one or more substituents each independently selected from the group consisting of CH₃, cyclopropyl, and Aryl2, said heteroatom being an oxygen atom,
a 5- to 9-membered fused bicyclic unsaturated or saturated ring, in particular a saturated heterocycle fused with an aromatic ring which may be optionally substituted with OCH₃,
a 5- to 9-membered bridged bicyclic unsaturated or saturated ring optionally substituted with 1, 2 or 3 CH₃ substituents,
a C₅₋₁₂spirocycloalkyl, and
cubanyl;
   wherein Aryl1 is selected from the group consisting of phenyl, monocyclic heteroaryl, and bicyclic heteroaryl which is in particular an aromatic ring fused to a saturated ring or an aromatic ring fused to another aromatic ring, said Aryl1 being optionally substituted with CH₃;
   wherein Aryl2 is selected from the group consisting of phenyl, monocyclic heteroaryl, and bicyclic heteroaryl, said Aryl2 being optionally substituted with one or more substituents each independently selected from the group consisting of halogens, CF₃, CF₂H, CH₂F, C₁₋₄alkyl, C₃₋₆cycloalkyl, CN, CONR¹⁸R¹⁹, OH, OCF₃, OCF₂H, OCH₂F, OC₁₋₄alkyl, OC₃₋₆cycloalkyl, SO₂CH₃, imidazolyl optionally substituted with CH₃, phenyl optionally substituted with fluoro, and triazolyl;
   wherein R¹⁸ and R¹⁹ are independently selected from the group consisting of hydrogen, C₁₋₆alkyl and C₃₋₆cycloalkyl;
   or wherein R′ and R″ together form a cycle or cycle system selected from the group consisting of
a C₃₋₈cycloalkyl ring,
a C₃₋₈cycloalkyl ring containing a heteroatom, said heteroatom being an oxygen atom, and optionally being substituted with CH₃,
a C₃₋₈cycloalkyl ring substituted with one or more substituents each independently selected from the group consisting of C₁₋₆alkyl, phenyl, C₂₋₆alkynyl and C₃₋₆cycloalkyl,
a C₃₋₈cycloalkyl ring containing a heteroatom and being substituted with one or more substituents each independently selected from the group consisting of C₁₋₆alkyl, CN, phenyl, C₂₋₆alkynyl and C₃₋₆cycloalkyl, said heteroatom being an oxygen atom,
a C₅₋₁₂-spirocycloalkyl optionally substituted with CH₃, and
a C₅₋₆ bridged bicyclic saturated ring system;
   R⁵ is selected from the group consisting of hydrogen, C₁₋₆alkyl, C₂₋₃alkenyl, Cycle2 and Aryl3;
   wherein C₁₋₆alkyl is optionally substituted with one or more substituents each independently selected from the group consisting of phenyl, methoxyphenyl, OC₁₋₆alkyl, NHSO₂CH₃, C₃₋₆cycloalkyl, and C₃₋₆cycloalkyl containing a heteroatom, said heteroatom being an oxygen atom;
   wherein Cycle2 is selected from the group consisting of
C₃₋₆cycloalkyl,
C₃₋₆cycloalkyl containing SO₂ or a heteroatom, the heteroatom being selected from the group consisting of oxygen and nitrogen,
C₃₋₆cycloalkyl substituted with CO₂R^{20a}, CONHR^{20b} or SO₂C₁₋₆alkyl,
C₃₋₆cycloalkyl containing SO₂ or a heteroatom and being substituted with CO₂R^{20a}, CONHR^{20b} or SO₂C₁₋₆alkyl, the heteroatom being selected from the group consisting of oxygen and nitrogen,
a 5-membered bridged bicyclic saturated ring substituted with CO₂C₁₋₆alkyl or CONHR^{20b},
cubanyl optionally substituted with CO₂C₁₋₆alkyl or CONHR^{20b},
isoindoline-1-one, and
indoline-2-one;
   wherein R^{20a} is hydrogen or C₁₋₆alkyl;
   wherein R^{20b} is C₁₋₆alkyl or C₃₋₆cycloalkyl;
   wherein Aryl3 is selected from the group consisting of phenyl, monocyclic heteroaryl, and bicyclic heteroaryl, said Aryl3 being optionally substituted with one or more substituents each independently selected from the group consisting of halogen, C₁₋₆alkyl, CF₃, CF₂H, CH₂F, CN, OC₁₋₆alkyl, OCF₃, OCF₂H, OCH₂F, OC₃₋₆cycloalkyl, SO₂R²¹, SO₂NHR²², CO₂R²³, COR²⁴, CONR²⁵R²⁶, NHR²⁷, NHCOR²⁸, Cycle3 and Aryl4;
   wherein R²¹ is C₁₋₆alkyl or C₃₋₆cycloalkyl;
   wherein R²² is C₁₋₆alkyl or pyridine;
   wherein R²³ is hydrogen or C₁₋₆alkyl;
   wherein R²⁴ is selected from the group consisting of C₁₋₆alkyl, C₅₋₆heterocycle in particular C₅₋₆heterocycloalkyl and C₅₋₆heterocycle, in particular C₅₋₆heterocycloalkyl, substituted with CH₃;
   wherein R²⁵ is hydrogen or CH₃;
   wherein R²⁶ is selected from the group consisting of
hydrogen,
C₁₋₆alkyl,
C₁₋₆alkyl optionally substituted with one or more substituents each independently selected from the group consisting of OH, OCH₃, NH₂, CO₂H, C₃₋₆heterocycloalkyl and C₃₋₆heterocycloalkyl substituted with CH₃,
C₃₋₆cycloalkyl;
C₃₋₆cycloalkyl containing a heteroatom, said heteroatom being an oxygen atom;
C₃₋₆cycloalkyl substituted with CO₂H; and
C₃₋₆cycloalkyl containing a heteroatom and being substituted with CO₂H, said heteroatom being an oxygen atom;
   wherein R²⁷ is selected from the group consisting of
C₁₋₆alkyl,
C₁₋₆alkyl substituted with C₃₋₆heterocycloalkyl, and
C₃₋₆heterocycloalkyl;
   wherein R²⁸ is C₁₋₆alkyl or C₃₋₆cycloalkyl;
   wherein Cycle3 is selected from the group consisting of
C₃₋₆cycloalkyl,
C₃₋₆heterocycloalkyl,
C₃₋₆heterocycloalkyl substituted with one or more substituents each independently selected from the group consisting of OH, CH₂OH, CO₂R²⁹, NHCH₃ or NHCO₂t-Bu; and
imidazolidin-4-one substituted with CH₃;
   wherein R²⁹ is hydrogen or C₁₋₆alkyl;
   wherein Aryl4 is selected from the group consisting of monocyclic heteroaryl and bicyclic heteroaryl, said monocyclic or bicyclic heteroaryl being optionally substituted with one or two substituents each independently selected from the group consisting of halogens, CF₃, CH₂F, C₁₋₆alkyl, C₃₋₆cycloalkyl, OCF₃, OCH₂F, OC₁₋₆alkyl, OC₃₋₆cycloalkyl, CO₂R³⁰, SO₂CH₃, and morpholine;
   wherein R³⁰ is hydrogen or C₁₋₆alkyl;
   wherein R′, R″ and R⁵ are not all hydrogen; and R⁵ is not CH(Ph)₂ when R⁴ is NH₂; and
   wherein R⁶ is hydrogen, CH₃, CF₃ or CF₂H;
   or a pharmaceutically acceptable salt thereof,
   with the proviso that the compound is not
2-(dimethylamino)-5,6,7,8-tetrahydro-7-[(2-methylimidazo[1,2-a]pyrimidin-3-yl)carbonyl]-Pyrido[3,4-d]pyrimidin-4(3H)-one,
7-[(2,3-dihydro-1,4-benzodioxin-6-yl)carbonyl]-2-(dimethylamino)-5,6,7,8-tetrahydro-pyrido[3,4-d]pyrimidin-4(3H)-one,
2-(dimethylamino)-5,6,7,8-tetrahydro-7-[(4,5,6,7-tetrahydrobenzo[b]thien-3-yl)carbonyl]-pyrido[3,4-d]pyrimidin-4(3H)-one,
5,6,7,8-tetrahydro-2-(1-piperidinyl)-7-(4-thiazolylcarbonyl)-pyrido[3,4-d]pyrimidin-4(3H)-one,
7-(2-fluorobenzoyl)-5,6,7,8-tetrahydro-2-(1-piperidinyl)-pyrido[3,4-d]pyrimidin-4(3H)-one,
5,6,7,8-tetrahydro-2-(4-methyl-1-piperidinyl)-7-(4-thiazolylcarbonyl)-pyrido[3,4-d]pyrimidin-4(3H)-one,
7-(3,4-dimethoxybenzoyl)-5,6,7,8-tetrahydro-2-(4-methyl-1-piperidinyl)-pyrido[3,4-d]pyrimidin-4(3H)-one,
5,6,7,8-tetrahydro-7-[(5-methyl-1-propyl-1H-pyrazol-4-yl)carbonyl]-2-(4-morpholinyl)-pyrido[3,4-d]pyrimidin-4(3H)-one,
7-[(1-ethyl-3-methyl-1H-pyrazol-4-yl)carbonyl]-5,6,7,8-tetrahydro-2-(4-methyl-1-piperidinyl)-pyrido[3,4-d]pyrimidin-4(3H)-one,
7-(2-furanylcarbonyl)-5,6,7,8-tetrahydro-2-(4-methyl-1-piperidinyl)-pyrido[3,4-d]pyrimidin-4(3H)-one,
7-(3,4-dimethoxybenzoyl)-5,6,7,8-tetrahydro-2-(1-pyrrolidinyl)-pyrido[3,4-d]pyrimidin-4(3H)-one,
5,6,7,8-tetrahydro-7-(pyrazolo[1,5-a]pyrimidin-3-ylcarbonyl)-2-(1-pyrrolidinyl)-pyrido[3,4-d]pyrimidin-4(3H)-one,
5,6,7,8-tetrahydro-7-[(5-methyl-1-propyl-1H-pyrazol-4-yl)carbonyl]-2-(1-piperidinyl)-pyrido[3,4-d]pyrimidin-4(3H)-one,
7-(3,4-dimethoxybenzoyl)-5,6,7,8-tetrahydro-2-(1-piperidinyl)-pyrido[3,4-d]pyrimidin-4(3H)-one,
5,6,7,8-tetrahydro-7-(2-pyridinylcarbonyl)-2-(1-pyrrolidinyl)-pyrido[3,4-d]pyrimidin-4(3H)-one,
2-(2,6-dimethyl-4-morpholinyl)-5,6,7,8-tetrahydro-7-(2-thienylcarbonyl)-pyrido[3,4-d]pyrimidin-4(3H)-one,
7-(cyclohexylcarbonyl)-5,6,7,8-tetrahydro-2-(1-pyrrolidinyl)-pyrido[3,4-d]pyrimidin-4(3H)-one,
5,6,7,8-tetrahydro-2-(4-methyl-1-piperidinyl)-7-[(5-methyl-1-propyl-1H-pyrazol-4-yl)carbonyl]-pyrido[3,4-d]pyrimidin-4(3H)-one,
7-(2-furanylcarbonyl)-5,6,7,8-tetrahydro-2-(2-methyl-1-piperidinyl)-pyrido[3,4-d]pyrimidin-4(3H)-one,
5,6,7,8-tetrahydro-2-(1-piperidinyl)-7-(2-pyrazinylcarbonyl)-pyrido[3,4-d]pyrimidin-4(3H)-one,
5,6,7,8-tetrahydro-2-(2-methyl-1-piperidinyl)-7-(2-thienylcarbonyl)-pyrido[3,4-d]pyrimidin-4(3H)-one,
2-(2,6-dimethyl-4-morpholinyl)-7-(2-fluorobenzoyl)-5,6,7,8-tetrahydro-pyrido[3,4-d]pyrimidin-4(3H)-one,
5,6,7,8-tetrahydro-2-(1-pyrrolidinyl)-7-(5-quinoxalinylcarbonyl)-pyrido[3,4-d]pyrimidin-4(3H)-one,
7-[(1,2-dimethyl-1H-benzimidazol-5-yl)carbonyl]-5,6,7,8-tetrahydro-2-(4-morpholinyl)-pyrido[3,4-d]pyrimidin-4(3H)-one,
5,6,7,8-tetrahydro-2-(4-morpholinyl)-7-[(4,5,6,7-tetrahydrobenzo[b]thien-3-yl)carbonyl]-pyrido[3,4-d]pyrimidin-4(3H)-one,
5,6,7,8-tetrahydro-2-(4-methyl-1-piperidinyl)-7-[(3-methyl-1H-pyrazol-4-yl)carbonyl]-pyrido[3,4-d]pyrimidin-4(3H)-one,
7-(2H-1-benzopyran-3-ylcarbonyl)-5,6,7,8-tetrahydro-2-(4-morpholinyl)-pyrido[3,4-d]pyrimidin-4(3H)-one,
5,6,7,8-tetrahydro-2-(4-methyl-1-piperidinyl)-7-[(4,5,6,7-tetrahydro-1H-indazol-3-yl)carbonyl]-pyrido[3,4-d]pyrimidin-4(3H)-one,
2-(2,6-dimethyl-4-morpholinyl)-5,6,7,8-tetrahydro-7-(1H-pyrazol-3-ylcarbonyl)-pyrido[3,4-d]pyrimidin-4(3H)-one,
5,6,7,8-tetrahydro-7-(1H-pyrazol-3-ylcarbonyl)-2-(1-pyrrolidinyl)-pyrido[3,4-d]pyrimidin-4(3H)-one,
7-(2-furanylcarbonyl)-5,6,7,8-tetrahydro-2-(1-pyrrolidinyl)-pyrido[3,4-d]pyrimidin-4(3H)-one,
7-[(1,3-dimethyl-1H-pyrazol-4-yl)carbonyl]-5,6,7,8-tetrahydro-2-(4-methyl-1-piperidinyl)-pyrido[3,4-d]pyrimidin-4(3H)-one,
7-[(1-ethyl-1H-pyrazol-4-yl)carbonyl]-5,6,7,8-tetrahydro-2-(1-pyrrolidinyl)-pyrido[3,4-d]pyrimidin-4(3H)-one,
5,6,7,8-tetrahydro-2-(4-methyl-1-piperidinyl)-7-[(4-methyl-5-thiazolyl)carbonyl]-pyrido[3,4-d]pyrimidin-4(3H)-one,
5,6,7,8-tetrahydro-7-[[5-(2-methylpropyl)-3-isoxazolyl]carbonyl]-2-(1-pyrrolidinyl)-pyrido[3,4-d]pyrimidin-4(3H)-one,
5,6,7,8-tetrahydro-7-(3-pyridinylcarbonyl)-2-(1-pyrrolidinyl)-pyrido[3,4-d]pyrimidin-4(3H)-one,
7-(2-fluorobenzoyl)-5,6,7,8-tetrahydro-2-(1-pyrrolidinyl)-pyrido[3,4-d]pyrimidin-4(3H)-one,
7-(cyclohexylcarbonyl)-2-(2,6-dimethyl-4-morpholinyl)-5,6,7,8-tetrahydro-pyrido[3,4-d]pyrimidin-4(3H)-one,
2-(dimethylamino)-5,6,7,8-tetrahydro-7-(5-quinoxalinylcarbonyl)-pyrido[3,4-d]pyrimidin-4(3H)-one,
2-(dimethylamino)-5,6,7,8-tetrahydro-7-[(2-propyl-4-thiazolyl)carbonyl]-pyrido[3,4-d]pyrimidin-4(3H)-one,
7-[(3-chloro-2-thienyl)carbonyl]-2-(dimethylamino)-5,6,7,8-tetrahydro-pyrido[3,4-d]pyrimidin-4(3H)-one,
2-(dimethylamino)-7-[(1-ethyl-3-methyl-3-piperidinyl)carbonyl]-5,6,7,8-tetrahydro-pyrido[3,4-d]pyrimidin-4(3H)-one,
2-(dimethylamino)-5,6,7,8-tetrahydro-7-[(tetrahydro-2-furanyl)carbonyl]-pyrido[3,4-d]pyrimidin-4(3H)-one,
2-(dimethylamino)-5,6,7,8-tetrahydro-7-(4Hthieno[3,2-b]pyrrol-5-ylcarbonyl)-pyrido[3,4-d]pyrimidin-4(3H)-one,
7-[(2,6-dimethoxy-3-pyridinyl)carbonyl]-2-(dimethylamino)-5,6,7,8-tetrahydro-pyrido[3,4-d]pyrimidin-4(3H)-one,
7-[(4-chloro-1H-pyrazol-3-yl)carbonyl]-2-(dimethylamino)-5,6,7,8-tetrahydro-pyrido[3,4-d]pyrimidin-4(3H)-one,
2-(dimethylamino)-7-[(1-ethyl-5-methyl-1Hpyrazol-4-yl)carbonyl]-5,6,7,8-tetrahydro-pyrido[3,4-d]pyrimidin-4(3H)-one,
2-(dimethylamino)-5,6,7,8-tetrahydro-7-(2-hydroxybenzoyl)-pyrido[3,4-d]pyrimidin-4(3H)-one,
2-(dimethylamino)-5,6,7,8-tetrahydro-7-[(3-methylbenzo[b]thien-2-yl)carbonyl]-pyrido[3,4-d]pyrimidin-4(3H)-one,
7-[(3-cyclohexyl-1H-pyrazol-4-yl)carbonyl]-2-(dimethylamino)-5,6,7,8-tetrahydro-pyrido[3,4-d]pyrimidin-4(3H)-one,
2-(dimethylamino)-5,6,7,8-tetrahydro-7-[(7-methylpyrazolo[1,5-a]pyrimidin-6-yl)carbonyl]-pyrido[3,4-d]pyrimidin-4(3H)-one,
7-[(6,7-dihydro-2-methoxy-5H-cyclopenta[b]pyridin-3-yl)carbonyl]-2-(dimethylamino)-5,6,7,8-tetrahydro-pyrido[3,4-d]pyrimidin-4(3H)-one,
2-(dimethylamino)-7-[(2-ethyl-4-methyl-5-oxazolyl)carbonyl]-5,6,7,8-tetrahydro-pyrido[3,4-d]pyrimidin-4(3H)-one,
7-(2-chloro-3-methylbenzoyl)-2-(dimethylamino)-5,6,7,8-tetrahydro-pyrido[3,4-d]pyrimidin-4(3H)-one,
2-(dimethylamino)-5,6,7,8-tetrahydro-7-[2-(trifluoromethyl)benzoyl]-pyrido[3,4-d]pyrimidin-4(3H)-one,
7-[(1,2-dihydro-1,4,6-trimethyl-2-oxo-3-pyridinyl)carbonyl]-2-(dimethylamino)-5,6,7,8-tetrahydro-pyrido[3,4-d]pyrimidin-4(3H)-one,
2-(dimethylamino)-5,6,7,8-tetrahydro-7-[(6-methylimidazo[2,1-b]thiazol-5-yl)carbonyl]-pyrido[3,4-d]pyrimidin-4(3H)-one,
2-(dimethylamino)-5,6,7,8-tetrahydro-7-[(1,4,5,6-tetrahydro-3-cyclopentapyrazolyl)carbonyl]-pyrido[3,4-d]pyrimidin-4(3H)-one,
7-[(2,3-dihydrothieno[3,4-b]-1,4-dioxin-5-yl)carbonyl]-2-(dimethylamino)-5,6,7,8-tetrahydro-pyrido[3,4-d]pyrimidin-4(3H)-one,
2-(dimethylamino)-5,6,7,8-tetrahydro-7-[[3-(2-methylpropyl)-5-isoxazolyl]carbonyl]-pyrido[3,4-d]pyrimidin-4(3H)-one,
2-(dimethylamino)-5,6,7,8-tetrahydro-7-(4-propylbenzoyl)-pyrido[3,4-d]pyrimidin-4(3H)-one,
7-[(6-chloroimidazo[1,2-a]pyridin-2-yl)carbonyl]-2-(dimethylamino)-5,6,7,8-tetrahydro-pyrido[3,4-d]pyrimidin-4(3H)-one,
2-(dimethylamino)-7-[[1-ethyl-3-(1-methylethyl)-1H-pyrazol-5-yl]carbonyl]-5,6,7,8-tetrahydro-pyrido[3,4-d]pyrimidin-4(3H)-one,
2-(dimethylamino)-5,6,7,8-tetrahydro-7-[(1-methyl-1H-indol-2-yl)carbonyl]-pyrido[3,4-d]pyrimidin-4(3H)-one,
2-(dimethylamino)-5,6,7,8-tetrahydro-7-[(2R)-2-pyrrolidinylcarbonyl]-pyrido[3,4-d]pyrimidin-4(3H)-one,
2-(dimethylamino)-7-[(5,7-dimethyl-1,2,4-triazolo[4,3-a]pyrimidin-3-yl)carbonyl]-5,6,7,8-tetrahydro-pyrido[3,4-d]pyrimidin-4(3H)-one,
2-(dimethylamino)-5,6,7,8-tetrahydro-7-[(7-methyl-2-benzofuranyl)carbonyl]-pyrido[3,4-d]pyrimidin-4(3H)-one,
2-(dimethylamino)-5,6,7,8-tetrahydro-7-[[5-(1-methylethyl)-3-isoxazolyl]carbonyl]-pyrido[3,4-d]pyrimidin-4(3H)-one,
2-(dimethylamino)-7-[(5,7-dimethyl[1,2,4]triazolo[1,5-a]pyrimidin-2-yl)carbonyl]-5,6,7,8-tetrahydro-pyrido[3,4-d]pyrimidin-4(3H)-one,
2-(dimethylamino)-5,6,7,8-tetrahydro-7-[[4-methyl-2-(1-methylethyl)-5-pyrimidinyl]carbonyl]-pyrido[3,4-d]pyrimidin-4(3H)-one,
2-(dimethylamino)-5,6,7,8-tetrahydro-7-[(4,5,6,7-tetrahydro-5-methyl-2H-indazol-3-yl)carbonyl]-pyrido[3,4-d]pyrimidin-4(3H)-one,
7-(5-chloro-2-methoxybenzoyl)-2-(dimethylamino)-5,6,7,8-tetrahydro-pyrido[3,4-d]pyrimidin-4(3H)-one,
2-(dimethylamino)-5,6,7,8-tetrahydro-7-[(4,5,6,7-tetrahydro-5-methyl-1H-pyrazolo[4,3-c]pyridin-3-yl)carbonyl]-pyrido[3,4-d]pyrimidin-4(3H)-one,
2-(dimethylamino)-5,6,7,8-tetrahydro-7-[(1,2,3,4-tetrahydro-8-quinolinyl)carbonyl]-pyrido[3,4-d]pyrimidin-4(3H)-one,
2-(dimethylamino)-7-[[2-(ethylamino)-4-methyl-5-thiazolyl]carbonyl]-5,6,7,8-tetrahydro-pyrido[3,4-d]pyrimidin-4(3H)-one,
2-(dimethylamino)-7-[(2,7-dimethylpyrazolo[1,5-a]pyrimidin-5-yl)carbonyl]-5,6,7,8-tetrahydro-pyrido[3,4-d]pyrimidin-4(3H)-one,
5,6,7,8-tetrahydro-2-(4-methyl-1-piperidinyl)-7-(1Hpyrazol-3-ylcarbonyl)-pyrido[3,4-d]pyrimidin-4(3H)-one,
7-[[3-(1,1-dimethylethyl)-1-methyl-1H-pyrazol-5-yl]carbonyl]-5,6,7,8-tetrahydro-2-(4-morpholinyl)-pyrido[3,4-d]pyrimidin-4(3H)-one,
7-(2-fluorobenzoyl)-5,6,7,8-tetrahydro-2-(4-morpholinyl)-pyrido[3,4-d]pyrimidin-4(3H)-one,
7-[(1-ethyl-1H-pyrazol-4-yl)carbonyl]-5,6,7,8-tetrahydro-2-(4-methyl-1-piperidinyl)-pyrido[3,4-d]pyrimidin-4(3H)-one,
7-(cyclohexylcarbonyl)-5,6,7,8-tetrahydro-2-(4-morpholinyl)-pyrido[3,4-d]pyrimidin-4(3H)-one,
7-[(1-ethyl-5-methyl-1H-pyrazol-4-yl)carbonyl]-5,6,7,8-tetrahydro-2-(1-pyrrolidinyl)-pyrido[3,4-d]pyrimidin-4(3H)-one,
7-[(1,5-dimethyl-1H-pyrazol-4-yl)carbonyl]-5,6,7,8-tetrahydro-2-(1-pyrrolidinyl)-pyrido[3,4-d]pyrimidin-4(3H)-one,
5,6,7,8-tetrahydro-2-(4-methyl-1-piperidinyl)-7-(2-pyrazinylcarbonyl)-pyrido[3,4-d]pyrimidin-4(3H)-one,
5,6,7,8-tetrahydro-2-(1-piperidinyl)-7-[(1,4,5,6-tetrahydro-3-cyclopentapyrazolyl)carbonyl]-pyrido[3,4-d]pyrimidin-4(3H)-one,
7-[(1-ethyl-5-methyl-1H-pyrazol-4-yl)carbonyl]-5,6,7,8-tetrahydro-2-(4-methyl-1-piperidinyl)-pyrido[3,4-d]pyrimidin-4(3H)-one,
5,6,7,8-tetrahydro-2-(1-piperidinyl)-7-[(4,5,6,7-tetrahydro-1H-indazol-3-yl)carbonyl]-pyrido[3,4-d]pyrimidin-4(3H)-one,
7-[(2,3-dihydro-1,4-benzodioxin-6-yl)carbonyl]-5,6,7,8-tetrahydro-2-(1-pyrrolidinyl)-pyrido[3,4-d]pyrimidin-4(3H)-one,
7-(3,5-difluorobenzoyl)-5,6,7,8-tetrahydro-2-(1-pyrrolidinyl)-pyrido[3,4-d]pyrimidin-4(3H)-one,
5,6,7,8-tetrahydro-2-(1-pyrrolidinyl)-7-(2-thienylcarbonyl)-pyrido[3,4-d]pyrimidin-4(3H)-one,
5,6,7,8-tetrahydro-7-(1-isoquinolinylcarbonyl)-2-(4-morpholinyl)-pyrido[3,4-d]pyrimidin-4(3H)-one,
5,6,7,8-tetrahydro-7-(1-isoquinolinylcarbonyl)-2-(1-pyrrolidinyl)-pyrido[3,4-d]pyrimidin-4(3H)-one,
7-[(1-ethyl-1H-pyrazol-4-yl)carbonyl]-5,6,7,8-tetrahydro-2-(1-piperidinyl)-pyrido[3,4-d]pyrimidin-4(3H)-one,
5,6,7,8-tetrahydro-2-(1-piperidinyl)-7-(2-thienylcarbonyl)-pyrido[3,4-d]pyrimidin-4(3H)-one,
7-[(1,5-dimethyl-1H-pyrazol-4-yl)carbonyl]-5,6,7,8-tetrahydro-2-(4-methyl-1-piperidinyl)-pyrido[3,4-d]pyrimidin-4(3H)-one,
5,6,7,8-tetrahydro-2-(1-piperidinyl)-7-(1H-pyrazol-3-ylcarbonyl)-pyrido[3,4-d]pyrimidin-4(3H)-one, or
7-[(2-cyclopropyl-4-quinolinyl)carbonyl]-5,6,7,8-tetrahydro-2-(4-morpholinyl)-pyrido[3,4-d]pyrimidin-4(3H)-one.

Provided also is a compound of Formula (I)

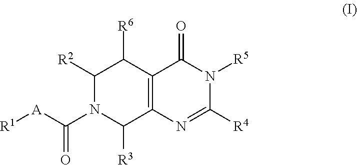

including any of its stereoisomers or tautomeric forms thereof, or a pharmaceutically acceptable salt thereof, wherein:
A is a bond or NH;
R¹ is a 5- to 10-membered monocyclic or bicyclic ring, more particularly a 5- to 9-membered monocyclic or bicyclic ring, wherein the 5- to 10-membered monocyclic or bicyclic ring, more particularly the 5- to 9-membered monocyclic or bicyclic ring, optionally contains 1 to 3 heteroatoms, the heteroatoms independently being selected from N, O and S;
wherein the 5- to 10-membered monocyclic or bicyclic ring, more particularly the 5- to 9-membered monocyclic or bicyclic ring is optionally substituted with one or more substituents selected from hydrogen, halogens, CN, CF₃, CF₂H, CFH₂, CF₂CH₃, C₁₋₆alkyl, OC₁₋₆alkyl, OCF₃, OCF₂H and C₃₋₄cycloalkyl;
R² is selected from the group consisting of hydrogen, C₁₋₆alkyl, CF₃, CHF₂, CH₂F, phenyl and fluorophenyl;
R³ is hydrogen;

### R⁴ is X—R′;

wherein X is NR″, S or O;
wherein R′ is hydrogen, C₁₋₄alkyl, C₁₋₆alkyl substituted with OH, or C₂₋₃alkenyl, when X is NR″;
wherein R′ is C₁₋₆alkyl, when X is S;
wherein R′ is C₁₋₆alkyl, when X is O;
wherein R″ is selected from the group consisting of hydrogen, Cycle1, Aryl1, C₂₋₄alkynyl, C₁₋₆alkyl and C₁₋₆alkyl substituted with one or more substituents selected from the group consisting of
fluoro,
OH,
CO₂R¹⁶,
OCONHR¹⁷,
C₃₋₆cycloalkyl, and C₃₋₆cycloalkyl substituted with one or more from among C₁₋₆alkyl,
N-acetyl piperidine,
benzo[d][1,3]dioxole and
Aryl2;
   wherein R¹⁶ is hydrogen or C₁₋₆alkyl;
   wherein R¹⁷ is C₁₋₆alkyl;
   wherein Cycle1 is selected from the group consisting of
C₃₋₈cycloalkyl
C₃₋₈cycloalkyl containing a heteroatom, said heteroatom being an oxygen atom,
C₃₋₈cycloalkyl substituted with one or more substituents selected from CH₃ and Aryl2,
C₃₋₈cycloalkyl containing a heteroatom and being substituted with one or more substituents selected from CH₃ and Aryl2, said heteroatom being an oxygen atom,
a 5-9 membered fused bicyclic unsaturated or saturated ring,
a 5-9 membered bridged bicyclic unsaturated or saturated ring, and
a C₅₋₁₂spirocycloalkyl;
   wherein Aryl1 is selected from the group consisting of phenyl, monocyclic heteroaryl, and bicyclic heteroaryl, said Aryl1 being optionally substituted with CH₃;
   wherein Aryl2 is selected from the group consisting of phenyl, monocyclic heteroaryl, and bicyclic heteroaryl, said Aryl2 being optionally substituted with one or more substituents selected from the group consisting of halogens, CF₃, CF₂H, CH₂F, C₁₋₄alkyl, C₃₋₆cycloalkyl, CN, CONR¹⁸R¹⁹, OCF₃, OCF₂H, OCH₂F, OC₁₋₄alkyl, OC₃₋₆cycloalkyl, and SO₂CH₃;
   wherein R¹⁸ and R¹⁹ are independently selected from the group consisting of hydrogen, C₁₋₆alkyl and C₃₋₆cycloalkyl;
   or wherein R′ and R″ together form a cycle selected from the group consisting of
a C₃₋₈cycloalkyl ring,
a C₃₋₈cycloalkyl ring containing a heteroatom, said heteroatom being an oxygen atom,
a C₃₋₈cycloalkyl ring substituted with one or more substituents selected from C₁₋₆alkyl, phenyl, C₂₋₆alkynyl and C₃₋₆cycloalkyl,
a C₃₋₈cycloalkyl ring containing a heteroatom and being substituted with one or more substituents selected from C₁₋₆alkyl, phenyl, C₂₋₆alkynyl and C₃₋₆cycloalkyl, said heteroatom being an oxygen atom and
a C₅₋₁₂-spirocycloalkyl;
   R⁵ is selected from the group consisting of hydrogen, C₁₋₆alkyl, C₂₋₃alkenyl, Cycle2 and Aryl3;
   wherein C₁₋₆alkyl is optionally substituted with one or more substituents selected from the group consisting of phenyl, methoxyphenyl, OC₁₋₆alkyl, NHSO₂CH₃,
   C₃₋₆cycloalkyl, and C₃₋₆cycloalkyl containing a heteroatom, said heteroatom being an oxygen atom;
   wherein Cycle2 is selected from the group consisting of
C₃₋₆cycloalkyl,
C₃₋₆cycloalkyl containing SO₂ or a heteroatom, the heteroatom being selected from the group consisting of oxygen and nitrogen,
C₃₋₆cycloalkyl substituted with CO₂R^{20a}, CONHC₁₋₆alkyl or SO₂C₁₋₆alkyl,
C₃₋₆cycloalkyl containing SO₂ or a heteroatom and being substituted with CO₂R^{20a}, CONHC₁₋₆alkyl or SO₂C₁₋₆alkyl, the heteroatom being selected from the group consisting of oxygen and nitrogen,
a 5-membered bridged bicyclic saturated ring substituted with CO₂C₁₋₆alkyl or CONHC₁₋₆Alkyl,
isoindoline-1-one, and
indoline-2-one;
   wherein R^{20a} is hydrogen or C₁₋₆alkyl;
   wherein Aryl3 is selected from the group consisting of phenyl, monocyclic heteroaryl, and bicyclic heteroaryl, said Aryl3 being optionally substituted with one or more substituents selected from the group consisting of halogen, C₁₋₆alkyl, CF₃, CF₂H, CH₂F, CN, OC₁₋₆alkyl, OCF₃, OCF₂H, OCH₂F, OC₃₋₆cycloalkyl, SO₂R²¹, SO₂NHR²², CO₂R²³, COR²⁴, CONR²⁵R²⁶, NHR²⁷, NHCOR²⁸, Cycle3 and Aryl4;
   wherein R²¹ is C₁₋₆alkyl or C₃₋₆cycloalkyl;
   wherein R²² is C₁₋₆alkyl or pyridine;
   wherein R²³ is hydrogen or C₁₋₆alkyl;
   wherein R²⁴ is selected from the group consisting of C₁₋₆alkyl, C₅₋₆heterocycle and C₅₋₆heterocycle substituted with CH₃;
   wherein R²⁵ is hydrogen or CH₃;
   wherein R²⁶ is selected from the group consisting of hydrogen,
C₁₋₆alkyl,
C₁₋₆alkyl optionally substituted with one or more substituents selected from the group consisting of OH, OCH₃, NH₂, CO₂H, C₃₋₆heterocycloalkyl and C₃₋₆heterocycloalkyl substituted with CH₃, C₃₋₄cycloalkyl;
C₃₋₄cycloalkyl containing a heteroatom, said heteroatom being an oxygen atom;
C₃₋₄cycloalkyl substituted with CO₂H; and
C₃₋₄cycloalkyl containing a heteroatom and being substituted with CO₂H, said heteroatom being an oxygen atom;
   wherein R²⁷ is selected from the group consisting of C₁₋₆alkyl,
C₁₋₆alkyl substituted with C₃₋₆heterocycloalkyl, and
C₃₋₆heterocycloalkyl;
   wherein R²⁸ is C₁₋₆alkyl or C₃₋₆cycloalkyl;
   wherein Cycle3 is selected from the group consisting of
C₃₋₆heterocycloalkyl,
C₃₋₆heterocycloalkyl substituted with one or more substituents selected from the group consisting of OH, CH₂OH, CO₂R²⁹, NHCH₃ or NHCO₂t-Bu; and imidazolidin-4-one substituted with CH₃;
   wherein R²⁹ is hydrogen or C₁₋₆alkyl;
   wherein Aryl4 is selected from the group consisting of monocyclic heteroaryl and bicyclic heteroaryl, said monocyclic or bicyclic heteroaryl being optionally substituted with one or two substituents selected from the group consisting of halogens, CF₃, CH₂F, C₁₋₆alkyl, C₃₋₆cycloalkyl, OCF₃, OCH₂F, OC₁₋₆alkyl, OC₃₋₆cycloalkyl, CO₂R³⁰, SO₂CH₃, and morpholine;
   wherein R³⁰ is hydrogen or C₁₋₆alkyl;
   wherein R′, R″ and R⁵ are not all hydrogen; and R⁵ is not CH(Ph)₂ when R⁴ is NH₂; and
   wherein R⁶ is hydrogen, CH₃, CF₃ or CF₂H.

The application relates to a pharmaceutical composition, which comprises at least one compound or a pharmaceutically acceptable salt thereof as defined herein, and which further comprises at least one pharmaceutically acceptable carrier.

In yet another aspect, the application pertains to the compound or pharmaceutically acceptable salt thereof as defined herein or the pharmaceutical composition as defined herein, for use as a medicament.

In yet another aspect, the application pertains to the compound or pharmaceutically acceptable salt thereof as defined herein, or the pharmaceutical composition as defined herein, for use in the prevention or treatment of an HBV infection or of an HBV-induced disease in mammal in need thereof.

In yet another aspect, the application pertains to the compound or pharmaceutically acceptable salt thereof as defined herein, or the pharmaceutical composition as defined herein, for use in the prevention or treatment of chronic Hepatitis B.

HBV-induced disease or condition includes progressive liver fibrosis, inflammation and necrosis leading to cirrhosis, end-stage liver disease, and hepatocellular carcinoma. Additionally, HBV acts as a helper virus to hepatitis delta virus (HDV), and it is estimated that more than 15 million people may be HBV/HDV co-infected worldwide, with an increased risk of rapid progression to cirrhosis and increased hepatic decompensation, than patients suffering from HBV alone (Hughes, S. A. et al. Lancet 2011, 378, 73-85). HDV, infects therefore subjects suffering from HBV infection. In a particular embodiment, the compounds of the invention may be used in the treatment and/or prophylaxis of HBV/HDV co-infection, or diseases associated with HBV/HDV co infection. Therefore, in a particular embodiment, the HBV infection is in particular HBV/HDV co-infection, and the mammal, in particular the human, may be HBV/HDV co-infected, or be at risk of HBV/HDV co infection.

In yet another aspect, the application pertains to the compound or pharmaceutically acceptable salt thereof as defined herein, or the pharmaceutical composition as defined herein, for use in the prevention or treatment of liver fibrosis, liver inflammation, liver necrosis, cirrhosis, end-stage liver disease or hepatocellular carcinoma.

In yet another aspect, the application pertains to a product comprising a first compound and a second compound as a combined preparation for simultaneous, separate or sequential use in the prevention or treatment of an HBV infection or of an HBV-induced disease in mammal in need thereof, wherein said first compound is different from said second compound, wherein said first compound is the compound or pharmaceutically acceptable salt thereof as defined herein or the pharmaceutical composition as defined herein, and wherein said second compound is another HBV inhibitor which is selected from the group consisting of: therapeutic agents selected from HBV combination drugs, HBV vaccines, HBV DNA polymerase inhibitors, immunomodulators, toll-like receptor (TLR) modulators, interferon alpha receptor ligands, hyaluronidase inhibitors, hepatitis b surface antigen (HBsAg) inhibitors, cytotoxic T-lymphocyte-associated protein 4 (ipi4) inhibitors, cyclophilin inhibitors, HBV viral entry inhibitors, antisense oligonucleotide targeting viral mRNA, short interfering RNAs (siRNA) and ddRNAi endonuclease modulators, ribonucleotide reductase inhibitors, HBV E antigen inhibitors, covalently closed circular DNA (cccDNA) inhibitors, famesoid X receptor agonists, HBV antibodies, CCR2 chemokine antagonists, thymosin agonists, cytokines, nucleoprotein modulators, retinoic acid-inducible gene 1 simulators, NOD2 stimulators, phosphatidylinositol 3-kinase (PI3K) inhibitors, indoleamine-2, 3-dioxygenase (IDO) pathway inhibitors, PD-1 inhibitors, PD-L1 inhibitors, recombinant thymosin alpha-1, bruton's tyrosine kinase (BTK) inhibitors, KDM inhibitors, HBV replication inhibitors, arginase inhibitors, and other HBV drugs.

In yet another aspect, the application pertains to a method for the preparation of a compound as defined herein, said method comprising the step of providing a compound according to Formula (XI):

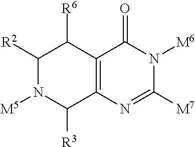

Formula (XI) or a tautomer thereof; wherein

M⁵ is selected from the group consisting of tert-butyloxycarbonyl, hydrogen, and R¹-A-C(O);

### M⁶ is H; and

M⁷ is selected from the group consisting of sulfhydryl, —SCH₃, —Cl, —S(O)CH₃, and R⁴; wherein when M⁵ is R¹-A-C(O)— and M⁶ is H, then M⁷ is not R⁴;
wherein when M⁵ and M⁶ are hydrogen, then M⁷ is not —NH₂;
wherein the method further comprises at least one of steps a) to h):
   a) contacting the compound according to Formula (XI) with a strong acid, more particularly hydrochloric acid or trifluoroacetic acid;
   b) contacting the compound according to Formula (XI) with R¹-A-C(O)-G, more particularly in the presence of a non-nucleophilic base;
   c) contacting the compound according to Formula (XI) with M¹⁰-H^{A}, more particularly in the presence of Cs₂CO₃, wherein M¹⁰ is selected from the group consisting of C₁₋₆alkyl, C₂₋₃alkenyl, and Cycle2; wherein C₁₋₆alkyl is optionally substituted with phenyl, methoxyphenyl, OC₁₋₆alkyl, NHSO₂CH₃, and C₃₋₆cycloalkyl optionally containing an oxygen atom; and wherein H^{A} is a halogen, more particularly Br;
   d) contacting the compound according to Formula (XI) with Aryl3-B(OH)₂, more particularly in the presence of copper(II) acetate;
   e) contacting the compound according to Formula (XI) with a methylating agent, more particularly methyl iodide;
   f) contacting the compound according to Formula (XI) with an oxidizing agent, more particularly meta-chloroperoxybenzoic acid;
   g) contacting the compound according to Formula (XI) with a chlorinating agent, more particularly thiophosgene;
   h) contacting the compound according to Formula (XI) with one of the reagents consisting of R′—S—H, R′—O—H and R′R″N—H, more particularly in the absence or in the presence of a non-nucleophilic base;
      wherein when M⁵ is tert-butyloxycarbonyl, the method comprises the steps a) and b);
      wherein when M⁵ is hydrogen, the method comprises step b);
      wherein when M⁷ is sulfhydryl the method comprises steps e), f) and h); or steps g) and h);
      wherein when M⁷ is —SCH₃, the method comprises steps f) and h);
      wherein when M⁷ is —Cl or —S(O)CH₃, the method comprises step h);
      wherein when the method comprises steps e) and/or f), the method does not comprise step g);
      wherein when the method comprises step c), the method does not comprise step d); and wherein:
      R¹, R², R³, R⁴, R⁵, R⁶, A, X, R′, R″, Cycle2, and Aryl3 are as defined herein;
      G is selected from the group consisting of OH and Cl;
when G is OH, step b) comprises contacting the compound according to Formula (XI) with R¹-A-C(O)-G in the presence of a coupling reagent; and
wherein the coupling reagent more particularly is 2-(1H-benzotriazol-1-yl)-1,1,3,3-tetramethyluronium hexafluorophosphate (HBTU).

In yet another aspect, the application relates to a method for the preparation of a compound as defined herein, wherein R⁵ is other than hydrogen, said method comprising the step of providing a compound according to Formula (XI):

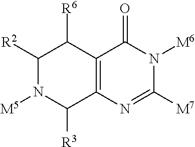

Formula (XI) or a tautomer thereof; wherein

M⁵ is R¹-A-C(O)—;
M⁶ is hydrogen; and
M⁷ is R⁴;
wherein the method further comprises at most one of steps a) and b):
a) contacting the compound according to Formula (XI) with M¹⁰-H^{A}, more particularly in the presence of Cs₂CO₃, wherein M¹⁰ is selected from the group consisting of C₁₋₆alkyl, C₂₋₃alkenyl, and Cycle2; wherein C₁₋₆alkyl is optionally substituted with phenyl, methoxyphenyl, OC₁₋₆alkyl, NHSO₂CH₃, and C₃₋₆cycloalkyl optionally containing an oxygen atom;
   and wherein H^{A} is a halogen, more particularly Br;
b) contacting the compound according to Formula (XII) with Aryl3-B(OH)₂, more particularly in the presence of copper(II) acetate;
   and wherein:
      R¹, R², R³, R⁴, R⁵, R⁶, A, Cycle2, and Aryl3 are as defined herein, with the proviso that R⁵ is not hydrogen.

In yet a further aspect, the application relates to a method for the preparation of a compound as defined herein, wherein R⁵ is other than hydrogen, said method comprising the step of providing a compound according to Formula (XI):

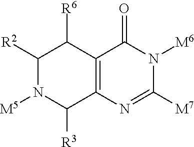

Formula (XI) or a tautomer thereof; wherein

M⁵ is selected from the group consisting of tert-butyloxycarbonyl, hydrogen, and R¹-A-C(O);
M⁶ is R⁵ and is other than hydrogen; and
M⁷ is selected from the group consisting of sulfhydryl, —SCH₃, —Cl, —S(O)CH₃, and R⁴;
wherein when M⁵ is R¹-A-C(O)—, then M⁷ is not R⁴;
wherein the method further comprises at least one of steps a) to f):
a) contacting the compound according to Formula (XIII) with a strong acid, more particularly hydrochloric acid or trifluoroacetic acid;
b) contacting the compound according to Formula (XIII) with R¹-A-C(O)-G, more particularly in the presence of a non-nucleophilic base;
c) contacting the compound according to Formula (XIII) with a methylating agent, more particularly methyl iodide;
d) contacting the compound according to Formula (XIII) with an oxidizing agent, more particularly meta-chloroperoxybenzoic acid;
e) contacting the compound according to Formula (XIII) with a chlorinating agent, more particularly thiophosgene;
f) contacting the compound according to Formula (XIII) with one of the reagents consisting of R′—S—H, R′—O—H and R′R″N—H, more particularly in the absence or in the presence of a non-nucleophilic base;
   wherein when M⁵ is tert-butyloxycarbonyl, the method comprises the steps a) and b);
   wherein when M⁵ is hydrogen, the method comprises step b);
   wherein when M⁷ is sulfhydryl the method comprises steps c), d) and f); or steps e) and f);
   wherein when M⁷ is —SCH₃, the method comprises steps d) and f);
   wherein when M⁷ is —Cl or —S(O)CH₃, the method comprises step f);
   wherein when the method comprises steps c) and/or d), the method does not comprise step e); and wherein:
   R¹, R², R³, R⁴, R⁵, R⁶, A, X, R′, R″, Cycle2, and Aryl3 are as defined herein, with the proviso that R⁵ is other than hydrogen;
   G is selected from the group consisting of OH and Cl;
when G is OH, step b) comprises contacting the compound according to Formula (XIII) with R¹-A-C(O)-G in the presence of a coupling reagent; and
wherein the coupling reagent more particularly is 2-(1H-benzotriazol-1-yl)-1,1,3,3-tetramethyluronium hexafluorophosphate (HBTU).

In yet another aspect, the application pertains to a process for the preparation of a compound as defined herein, comprising the steps of:
a) contacting a compound of Formula (XII) with R⁵—NH₂, wherein Formula (XII) is

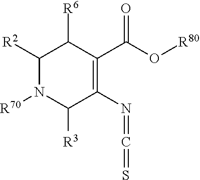

Formula (XII), to provide the compound of Formula (XIII):

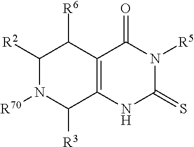

Formula (XIII);
b) contacting the compound of Formula (XIII) with a methylating agent, more particularly methyl iodide, to form a compound according to Formula (XIV):

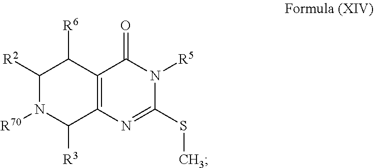

c) contacting the compound of Formula (XIV) with an oxidizing agent, more particularly meta-chloroperoxybenzoic acid, to form a compound of Formula (XV):

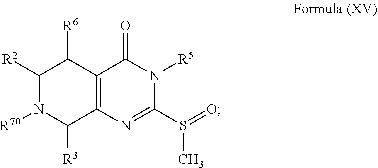

d) contacting the compound of Formula (XV) with R′R″N—H, more particularly in the presence of a non-nucleophilic base, to form a compound of Formula (XVI):

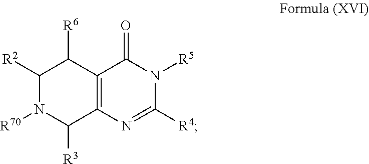

e) contacting the compound of Formula (XVI) with a strong acid, more particularly hydrochloric acid or trifluoroacetic acid, to form a compound of Formula (XVII):

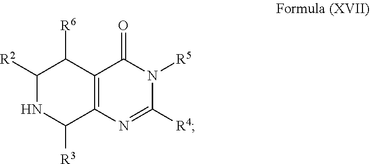

f) contacting the compound of Formula (XVII) with R¹-A-C(O)-G;
wherein:
   R¹, R², R³, R⁴, R⁵, R⁶, A, X, R′ and R″ are as defined herein;
   R⁷⁰ is tert-butyloxycarbonyl;
   R⁸⁰ is C₁₋₄alkyl and
   G is selected from the group consisting of OH and Cl;
   when G is OH, step f) comprises contacting the compound according to Formula (XVII) with R¹-A-C(O)-G in the presence of a coupling reagent, more particularly in the presence of a non-nucleophilic base; and
   wherein the coupling reagent is more particularly 2-(1H-benzotriazol-1-yl)-1,1,3,3-tetramethyluronium hexafluorophosphate (HBTU).

### DESCRIPTION OF THE INVENTION

Provided herein are compounds, e.g., the compounds of formula (I), or pharmaceutically acceptable salts thereof, which are notably useful in the treatment or prevention of HBV infection or of an HBV-associated (or HBV-induced) condition or disease in a subject in need thereof.

The compounds provided herein have potent antiviral activity, and are believed to exhibit favorable metabolic properties, tissue distribution, safety and pharmaceutical profiles, and to be suitable for use in humans. Disclosed compounds may modulate (e.g., accelerate, delay, inhibit, disrupt or reduce) normal viral capsid assembly or disassembly, bind capsid or alter metabolism of cellular polyproteins and precursors. The modulation may occur when the capsid protein is mature, or during viral infectivity. Disclosed compounds can be used in methods of modulating the activity or properties of HBV cccDNA, or the generation or release of HBV RNA particles from within an infected cell.

A compound of the application may accelerate the kinetics of HBV capsid assembly, thereby preventing or competing with the encapsidation of the Pol-pgRNA complex and thus blocking the reverse transcription of the pgRNA.

In an embodiment, the compounds described herein may be suitable for monotherapy and are effective against natural or native HBV strains and against HBV strains resistant to currently known drugs. In another embodiment, the compounds described herein are suitable for use in combination therapy.

### Definitions

Listed below are definitions of various terms used to describe this invention. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and peptide chemistry are those well-known and commonly employed in the art. Similarly, chemical structures or formulas are intended in accordance with the general knowledge in the field of chemistry; the combinations of groups and of substituents that they encompass are compliant with the general knowledge in the field of chemistry.

As used herein, the articles “a” and “an” refer to one or to more than one (i.e. to at least one) of the grammatical object of the article. By way of example, “an element” means one element or more than one element. Furthermore, use of the term “including” as well as other forms, such as “include”, “includes,” and “included,” is not limiting.

As used herein, the term “comprising”, which is synonymous with “including” or “containing”, is open-ended, and does not exclude additional, unrecited element(s), ingredient(s) or method step(s), whereas the term “consisting of” is a closed term, which excludes any additional element, step, or ingredient which is not explicitly recited.

As used herein, the term “essentially consisting of” is a partially open term, which does not exclude additional, unrecited element(s), step(s), or ingredient(s), as long as these additional element(s), step(s) or ingredient(s) do not materially affect the basic and novel properties of the invention.

As used herein, the term “comprising” (or “comprise(s)”) hence includes the term “consisting of” (“consist(s) of”), as well as the term “essentially consisting of” (“essentially consist(s) of”). Accordingly, the term “comprising” (or “comprise(s)”) is, in the present application, meant as more particularly encompassing the term “consisting of” (“consist(s) of”), and the term “essentially consisting of” (“essentially consist(s) of”).

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, including ±5%, ±1%, and ±0.1% from the specified value, as such variations are appropriate to perform the disclosed methods.

As used herein, the term “capsid assembly modulator” refers to a compound that disrupts or accelerates or inhibits or hinders or delays or reduces or modifies normal capsid assembly (e.g., during maturation) or normal capsid disassembly (e.g., during infectivity) or perturbs capsid stability, thereby inducing aberrant capsid morphology and function. In an embodiment, a capsid assembly modulator accelerates capsid assembly or disassembly, thereby inducing aberrant capsid morphology. In another embodiment, a capsid assembly modulator interacts (e.g. binds at an active site, binds at an allosteric site, modifies or hinders folding and the like) with the major capsid assembly protein (CA), thereby disrupting capsid assembly or disassembly. In yet another embodiment, a capsid assembly modulator causes a perturbation in structure or function of CA (e.g., ability of CA to assemble, disassemble, bind to a substrate, fold into a suitable conformation, or the like), which attenuates viral infectivity or is lethal to the virus.

As used herein, the term “treatment” or “treating” is defined as the application or administration of a therapeutic agent, i.e., a disclosed compound (alone or in combination with another pharmaceutical agent), to a patient, or application or administration of a therapeutic agent to an isolated tissue or cell line from a patient (e.g., for diagnosis or ex vivo applications), who has an HBV infection, a symptom of HBV infection or the potential to develop an HBV infection, with the purpose to cure, heal, alleviate, relieve, alter, remedy, ameliorate, improve or affect the HBV infection, the symptoms of HBV infection, or the potential to develop an HBV infection. Such treatments may be specifically tailored or modified, based on knowledge obtained from the field of pharmacogenomics.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual” or “subject” refers to a human or a non-human mammal. Non-human mammals include, for example, livestock and pets, such as ovine, bovine, porcine, canine, feline and murine mammals. Preferably, the patient, subject, or individual is human.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary, and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, the term “alkyl” by itself or as part of another substituent means, unless otherwise stated, a straight or branched chain hydrocarbon having the number of carbon atoms designated (i.e., C₁-C₃ alkyl or C₁₋₃alkyl means an alkyl having one to three carbon atoms, C₁-C₄ alkyl or C₁₋₄alkyl means an alkyl having one to four carbon) and includes straight and branched chains. Examples include methyl, ethyl, propyl, isopropyl, butyl, isobutyl, tert-butyl. Embodiments of alkyl generally include, but are not limited to, C₁-C₁₀alkyl, such as C₁-C₆alkyl, such as C₁-C₄alkyl.

As used herein, the term “alkenyl” by itself or as part of another substituent means, unless otherwise stated, a linear or branched chain of hydrocarbons comprising at least one carbon to carbon double bond, having the number of carbon atoms designated, i.e., C₂-C₄ alkenyl or C₂₋₄alkenyl means an alkenyl having two to four carbon atoms, C₄—C alkenyl or C₄₋₈alkenyl means an alkenyl having four to eight carbon atoms, C₁ alkenyl or C₁alkenyl means a linear or branched chain of hydrocarbons comprising one carbon, wherein the one carbon forms a double bond with a carbon of the main chain to which the C₁ alkenyl or C₁alkenyl is attached. In particular, an alkenyl group in relation to the application is a C₁-C₄ alkenyl or a C₁-C₃ alkenyl, more particularly a C₂-C₄ alkenyl, more particularly a C₂-C₃ alkenyl, more particularly a C₂ alkenyl, C₃ alkenyl, or C₄ alkenyl.

As used herein, the term “alkynyl” by itself or as part of another substituent means, unless otherwise stated, a linear or branched chain of hydrocarbons comprising at least one carbon to carbon triple bond, having the number of carbon atoms designated (i.e., C₂-C₄ alkynyl or C₂₋₄alkynyl means an alkynyl having two to four carbon atoms, C₄-C₈ alkynyl or C₄₋₈alkynyl means an alkynyl having four to eight carbon atoms. In particular, an alkynyl group in relation to the application is a C₂-C₆ alkynyl, more particularly a C₂-C₄ alkynyl, more particularly a C₂ alkynyl, C₃ alkynyl, or C₄ alkynyl.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, “oxo” represents ═O.

As used herein, the term “cycloalkyl” refers to a mono cyclic non-aromatic saturated radical, wherein each of the atoms forming the ring (i.e., skeletal atoms) is a carbon atom, unless such ring contains one or more heteroatoms if so further defined. C₃₋₈cycloalkyl include groups having 3 to 8 ring atoms. Such 3-8 membered saturated rings include, but are not limited to, cyclopropyl, cyclobutyl, cyclopentyl, cyclohexyl, cycloheptyl, cyclooctyl. A cycloalkyl radical consisting of carbon and hydrogen atoms can also be referred to as carbo-cycloalkyl.

As used herein, C₃₋₈cycloalkyl optionally is a heterocyclic group (which may also be denoted as a heterocycloalkyl group) comprising one or more heteroatoms, more in particular, one, two or three, even more in particular, one or two, and most particular, one. Said ring heteroatoms are each selected from O, S, and N. In an embodiment, each heterocyclic group has from 3 to 8 atoms in its ring system, with the proviso that the ring of said group does not contain two adjacent O or S atoms. The heterocyclic group can be attached to the remainder of the molecule, unless otherwise stated, at any heteroatom or carbon atom that affords a stable structure. If indicated, the heterocycle can be partially saturated. Particular examples of heterocyclic groups include, but are not limited to azetidinyl, pyrrolidinyl, pyrazolidinyl, tetrahydrofuranyl, dioxolanyl, piperidinyl, piperazinyl, morpholinyl, azepanyl, and oxazepanyl.

An example of a 3-membered heterocyclic group includes, and is not limited to, aziridine. Examples of 4-membered heterocyclic groups include, and are not limited to, azetidine and a beta lactam. Examples of 5-membered heterocyclic groups include, and are not limited to, pyrrolidine, oxazolidine and thiazolidinedione.

Examples of 6-membered heterocycloalkyl groups include, and are not limited to, piperidine, morpholine, and piperazine. Examples of 7-membered heterocycloalkyl groups include, and are not limited to, azepanyl, and oxazepanyl, e.g. 1,4-oxazepanyl.

Other non-limiting examples of heterocyclic groups include monocyclic groups such as aziridine, oxirane, thiirane, azetidine, oxetane, thietane, pyrrolidine, pyrroline, pyrazolidine, imidazoline, dioxolane, sulfolane, tetrahydrofuran, thiophene, piperidine, piperazine, morpholine, thiomorpholine.

As used herein, the term “monocyclic or bicyclic ring” refers to a mono- or bi-cyclic radical, wherein the atoms forming the ring (i.e. skeletal atoms) are one or more carbon atoms and optionally zero, one or more heteroatoms (such as S, O, N, B, P, more particularly S, O, N). The combinations of atoms and heteroatoms forming the ring are intended in accordance with the general knowledge in the field of chemistry. Unless the (chemical) context dictates otherwise, a monocyclic ring can be saturated, non-saturated, aromatic or non-aromatic.

Similarly, unless the (chemical) context dictates otherwise, a bicyclic ring can be saturated, unsaturated, aromatic, non-aromatic or a combination thereof, for example aromatic, or non-aromatic and saturated, or non-aromatic and non-saturated.

The term “unsaturated” or “non saturated” [ring] refers to the presence of double or triple bonds between the atoms forming the ring. An unsaturated ring may be aromatic or non-aromatic. The term “saturated” refers to the presence of single bonds (rather than multiple bonds) between the atoms forming the ring.

The term “aromatic” [ring] refers to a ring or a ring system comprising one or more cycles, wherein each of the one or more cycles is polyunsaturated and has aromatic character, i.e., has (4n+2) delocalized π (pi) electrons, where n is an integer. The cycle can e.g., be a carbocycle, or a heterocycle (wherein the heteroatom(s) is(are) for example chosen from among S, O, N, B and P, more particularly from among S, O and N).

The term “non-aromatic” [ring] refers to a ring, which does not comprise any cycle which would be polyunsaturated and would have aromatic character.

The disclosure, notably the disclosure on R¹, encompasses more particularly:
a monocycle, which is a 5- or 6-membered (monocyclic) aromatic ring, or
a polycycle, more particularly a bicycle, more particularly a 9-membered bicycle, wherein each cycle independently is aromatic or non-aromatic, and saturated or non-saturated, for example aromatic and non-saturated, or non-aromatic and saturated, or non-aromatic and non-saturated.

For example, a polycycle is a 9-membered bicycle, wherein the first cycle is aromatic (and non-saturated) and the second cycle is non-aromatic and non-saturated.

As used herein, the term “aryl,” employed alone or in combination with other terms, means, unless otherwise stated, a carbocyclic aromatic system containing one or more rings (typically one, two, or three rings), wherein such rings may be attached together in a pendent manner, such as a biphenyl, or may be fused, such as naphthalene. Examples of aryl groups include phenyl, anthracyl, and naphthyl. Preferred examples are phenyl (e.g., C₆-aryl) and biphenyl (e.g., C₁₂-aryl). In some embodiments, aryl groups have from six to sixteen carbon atoms. In some embodiments, aryl groups have from six to twelve carbon atoms (e.g., C₆-C₁₂-aryl). In some embodiments, aryl groups have six carbon atoms (e.g., C₆-aryl).

As used herein, the term “heteroaryl” or “heteroaromatic” refers to a heterocycle having aromatic character. By the reference to the aromatic character, the skilled person is aware of the customary limitations to the number of ring atoms. Generally, heteroaryl substituents may be defined by the number of carbon atoms, e.g., C₁₋₁₂heteroaryl, such as C₃₋₉ indicates the number of carbon atoms contained in the heteroaryl group without including the number of heteroatoms. For example, a C₁-C₉heteroaryl will include an additional one to four heteroatoms. A polycyclic heteroaryl may include one or more rings that are partially saturated. Non-limiting examples of heteroaryls include pyridyl, pyrazinyl, pyrimidinyl (including, e.g., 2- and 4-pyrimidinyl), pyridazinyl, thienyl, furyl, pyrrolyl (including, e.g., 2-pyrrolyl), imidazolyl, thiazolyl, oxazolyl, pyrazolyl (including, e.g., 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Non-limiting examples of polycyclic, such as bicyclic, heterocycles and heteroaryls include indolyl (including, e.g., 3-, 4-, 5-, 6- and 7-indolyl), indolinyl, quinolyl, tetrahydroquinolyl, isoquinolyl (including, e.g., 1- and 5-isoquinolyl), 1,2,3,4-tetrahydroisoquinolyl, cinnolinyl, quinoxalinyl (including, e.g., 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (including, e.g., 3-, 4-, 5-, 6- and 7-benzofuryl), 2,3-dihydrobenzofuryl, 1,2-benzisoxazolyl, benzothienyl (including, e.g., 3-, 4-, 5-, 6-, and 7-benzothienyl), benzoxazolyl, benzothiazolyl (including, e.g., 2-benzothiazolyl and 5-benzothiazolyl), purinyl, benzimidazolyl (including, e.g., 2-benzimidazolyl), benzotriazolyl, thioxanthinyl, carbazolyl, carbolinyl, acridinyl, pyrrolizidinyl, and quinolizidinyl.

Alternatively, aryl, heterocycles, heteroaryl and heteroaromatic groups may be referred to ring size by the total number of atoms in the ring or ring system, e.g. 5-membered, 6-membered if monocyclic, 9-membered, 10-membered if bicyclic, and so forth.

Within the context of this invention, bicyclic saturated carbo- or heterocyclic groups include fused, spiro and bridged saturated heterocycles.

As used herein, “spirocycloalkyl” refers to a radical that comprises a twisted structure of two or three rings, in particular at most two rings, that are linked together by one common atom, in particular a carbon atom. Thus, as used herein, ‘spiro bicyclic’ systems are cyclic systems wherein two cycles are joined at a single atom In particular, a spirocycloalkyl in relation to the application is a C₅₋₁₂spirocycloalkyl, more particularly a C₆₋₁₀spirocycloalkyl, more particularly a C₇₋₉spirocycloalkyl, more particularly a C₇spirocycloalkyl, more particularly spiro[3.3]heptyl. As used herein, a spirocycloalkyl may also contain at least one, in particular at most one, heteroatom, in particular N, more particularly 2-azaspiro[3.3]heptyl. Thus, spirocycloalkyl groups can be in particular spirocarbobicyclic or spiroheterobicyclic.

As used herein, “bridged bicyclic saturated ring” refers to a radical that has two saturated rings, and that contains a bridge, i.e. a single atom or an unbranched chain of atoms or a valence bond that connects two “bridgehead” atoms, i.e. two cycles that share more than two atoms. The bridgehead atoms are defined as any atom that is not a hydrogen, and that is part of the skeletal framework of the molecule. In particular, bridged bicyclic saturated rings in relation to the application are 5-membered bridged bicyclic saturated rings, in particular bicyclo[1.1.1]pentyl or bicyclo[2.1.0]pentyl, more particularly bicyclo[1.1.1]pentyl. Additional bridged bicyclic saturated rings include bicyclo[2.2.1]heptyl (norbornyl), and bridged bicyclic saturated heterocyclyl groups include 2-azabicyclo[2.1.1]hexyl. A particular example of a bridged polycyclic saturated ring is pentacyclo[4.2.0.0.0.0]octanyl (cubanyl). Fused bicyclic groups are two cycles that share two atoms and the bond between these atoms. Particular fused bicyclic systems include, but are not limited to for example, fused saturated carbocycles or heterocycles, e.g., 5-membered saturated heterocycle fused with a 6-membered saturated heterocycle, 6-membered saturated heterocycle fused with a 6-membered saturated heterocycle, or fused saturated and aromatic or partially saturated cycles, e.g. 5-membered heteroaryl fused with a 6-membered saturated carbo- or heterocycle, etc.

Whenever substituents are represented by chemical structure, “---” represents the bond of attachment to the remainder of the molecule.

Lines (such as “---”) drawn into a particular ring of a ring system indicate that the bond may be attached to any of the suitable ring atoms.

As used herein, the half maximal effective concentration (EC₅₀) is intended in accordance with its general meaning in the field. It may more particularly refer to the concentration of a compound which induces a response halfway between the baseline and maximum, typically after a specified exposure time. The EC₅₀ value is commonly used as a measure of a compound's potency, with a lower value generally indicating a higher potency.

The disclosed compounds may possess one or more stereocenters, and each stereocenter may exist independently in either R or S configuration. The stereochemical configuration may be assigned at indicated centers as (*R), (*S), (R*) or (S*) when the absolute stereochemistry is undetermined although the compound itself has been isolated as a single stereoisomer and is enantiomerically/diastereomerically pure. Compounds described herein are present in optically active or racemic forms. It is to be understood that the compounds described herein encompass racemic, optically-active, regioisomeric and stereoisomeric forms, or combinations thereof that possess the therapeutically useful properties described herein. When the absolute R or S stereochemistry of a compound cannot be determined, it can be identified by the retention time after chromatography under particular chromatographic conditions as determined by chromatographic column, eluent, etc. Additionally, the notation *S, and *R has been used herein to denote different atropoisomers, in the case where the atropoisomer(s) have been separated but the orientation has not been determined. An example of such atropoisomer is C36Bb.

A stereoisomeric form of a compound refers to all possible compounds made up of the same atoms bonded by the same sequence of bonds but having different three-dimensional structures which are not interchangeable.

Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. A mixture of one or more isomers can be utilized as the disclosed compound described herein. Compounds described herein may contain one or more chiral centers. These compounds can be prepared by any means, including stereoselective synthesis, enantioselective synthesis or separation of a mixture of enantiomers or diastereomers. Resolution of compounds and isomers thereof can be achieved by any means including, by way of non-limiting example, chemical processes, enzymatic processes, fractional crystallization, distillation, and chromatography.

The disclosed compounds may exist as tautomers. A “tautomer” refers to a proton-shift from one atom of the molecule to another atom of the same molecule. All tautomers are included within the scope of the compounds presented herein, although they may not be explicitly indicated in the above Formula (I). For example, when R⁵ is hydrogen, Formula (I) also covers the other tautomeric form

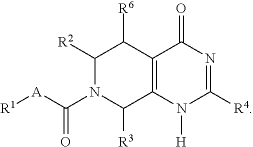

Compounds described herein also include isotopically-labeled compounds wherein one or more atoms are replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. Isotopically-labeled compounds can be useful in drug or substrate tissue distribution studies. Substitution with heavier isotopes such as deuterium may afford greater metabolic stability (which may lead to for example, increased in vivo half-life or reduced dosage requirements). Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds can be prepared by any suitable method or by processes using an appropriate isotopically-labeled reagent in place of the non-labeled reagent otherwise employed.

The compounds described herein may be labeled by other means, including, but not limited to, the use of chromophores or fluorescent moieties, bioluminescent labels, or chemiluminescent labels.

The compounds described herein, and other related compounds having different substituents can be synthesized using techniques and materials described herein and techniques known to the person of average skill in the art. General methods for the preparation of compound as described herein can be modified by the use of appropriate reagents and conditions, for the introduction of the various moieties found in the formula as provided herein.

Compounds described herein can be synthesized using any suitable procedures starting from compounds that are available from commercial sources or are prepared using procedures described herein.

The application relates to the subject matter described in the Summary above, more particularly to the following embodiments.

### Compounds

As already mentioned hereinabove, the present invention provides compounds of Formula(I). In a particular embodiment, the invention relates to a compound of Formula (I) as defined herein, wherein

R¹ is selected from the group consisting of
phenyl optionally substituted with one or more substituents, in particular 1, 2 or 3 substituents, each independently selected from the group consisting of halo, CN, CF₃, CF₂H, CH₂F, CF₂CH₃, C₁₋₆alkyl, OC₁₋₆alkyl, OCF₃, OCF₂H, and C₃₋₄cycloalkyl;
a 5- to 6-membered heteroaryl group containing 1 to 3 heteroatoms, each independently selected from the group consisting of N and O, and being optionally substituted with one or more substituents, in particular 1 to 2 substituents, each independently selected from the group consisting of halo, CN, CF₃, CF₂H, CH₂F, CF₂CH₃, C₁₋₆alkyl, OC₁₋₆alkyl, OCF₃, OCF₂H, and C₃₋₄cycloalkyl;
a 8- to 10-bicyclic heteroaromatic ring system containing 1 to 3 heteroatoms, each independently selected from the group consisting of N, S and O, and optionally substituted with one or more substituents, in particular 1, 2 or 3 substituents, each independently selected from the group consisting of halo, CN, CF₃, CF₂H, CH₂F, CF₂CH₃, C₁₋₆alkyl, OC₁₋₆alkyl, OCF₃, OCF₂H, and C₃₋₄cycloalkyl;
a 9- to 10-ring system wherein an aromatic ring is fused with a saturated ring, containing 1 to 3 heteroatoms, each independently selected from the group consisting of N and O, and optionally substituted with one or more substituents, in particular 1, 2 or 3 substituents, each independently selected from the group consisting of halo, C₁₋₆alkyl, and OC₁₋₆alkyl;
cubanyl optionally substituted with a substituent selected from the group consisting of halo and C₁₋₆alkyl,
or R¹ is selected from the group consisting of 1-methyl-2-oxo-1,3-dihydro-1H-benzo[d]imidazol-5-yl, 1-oxo-isoindolin-5-yl, and 1,1-dioxo-benzo[b]thiophen-5-yl; and all other variables are as defined herein.

In yet another embodiment, the invention relates to a compound of Formula (I) as defined herein, wherein

R¹ is selected from the group consisting of
phenyl optionally substituted with one or more substituents, in particular 1, 2 or 3 substituents, each independently selected from the group consisting of halo, CN, CF₃, CF₂H, CH₂F, CF₂CH₃, C₁₋₆alkyl, OC₁₋₆alkyl, OCF₃, OCF₂H, and C₃₋₄cycloalkyl;
a 5- to 6-membered heteroaryl group selected from pyridyl, thienyl, pyrrolyl and pyrazolyl, each of which is optionally substituted with one or more substituents, in particular 1 to 2 substituents, each independently selected from the group consisting of halo, CN, CF₃, C₁₋₆alkyl, OC₁₋₆alkyl, and C₃₋₄cycloalkyl, more in particular selected from the group consisting of halo, CN, CF₃, and C₁₋₆alkyl;
a 8- to 10-bicyclic heteroaromatic ring system selected from the group consisting of 1H-indolyl, 2,3-dihydro-1H-pyrrolo[3,2-b]pyridinyl, 1H-benzo[d]imidazolyl, benzo[b]thiophenyl, thieno[2,3-c]pyridinyl, imidazo[1,2-a]pyridinyl, imidazo[1,5-a]-pyridinyl, pyrazolo[1,5-a]pyridinyl, 1H-indazolyl, 1H-benzo[d][1,2,3]triazolyl, 1,1-dioxo-benzo[b]thiophenyl, [1,2,4]triazolo[1,5-a]pyridinyl, benzofuranyl, benzo[d]oxazolyl, benzo[d]thiazolyl, 4H-thieno[3,2-b]pyrrolyl, isoquinolinyl, each of which is optionally substituted with one or more substituents, in particular 1, 2 or 3 substituents, each independently selected from the group consisting of halo, CN, CF₃, C₁₋₆alkyl, OC₁₋₆alkyl, and OCF₃;
a 9- to 10-ring system selected from the group consisting of chromanyl, indolinyl, 2,3-dihydrobenzofuranyl, each optionally substituted with one or more, in particular 1 or 2 substituents, each independently selected from the group consisting of halo, C₁₋₆alkyl, and OC₁₋₆alkyl;
cubanyl optionally substituted with a halo substituent;
or R¹ is selected from the group consisting of 1-methyl-2-oxo-1,3-dihydro-1H-benzo[d]imidazol-5-yl, 1-oxo-isoindolin-5-yl, and 1,1-dioxo-benzo[b]thiophen-5-yl; and the rest of the variables are as defined herein.

In another embodiment, the invention relates to a compound of Formula (I) as defined herein, wherein R² is C₁₋₆alkyl, in particular methyl; and all other variables are as defined herein.

In an additional embodiment, the invention relates to a compound of Formula (I) as defined herein, wherein R² is C₁₋₆alkyl, in particular methyl having R stereoconfiguration; and all other variables are as defined herein.

In another embodiment, the invention relates to a compound of Formula (I) as defined herein, wherein R⁴ is selected from the group consisting of —OC₁₋₆alkyl, —SC₁₋₆alkyl and NR′R″, wherein

R′ is hydrogen, C₁₋₄alkyl, C₁₋₆alkyl substituted with OH, or C₂₋₃alkenyl; and
R″ is selected from the group consisting of hydrogen, Cycle1, Aryl1, C₂₋₄alkynyl, C₁₋₆alkyl and C₁₋₆alkyl substituted with one or more substituents each independently selected from the group consisting of
fluoro,
OH,
CO₂R¹⁶,
OCONHR¹⁷,
C₃₋₆cycloalkyl,
C₃₋₆cycloalkyl substituted with one or more from among C₁₋₆alkyl,
N-acetyl piperidine,
cubanyl,
benzo[d][1,3]dioxole, and
Aryl2;
   wherein R¹⁶ is hydrogen or C₁₋₆alkyl;
   wherein R¹⁷ is C₁₋₆alkyl;
   wherein Cycle1 is selected from the group consisting of
C₃₋₈cycloalkyl
C₃₋₈cycloalkyl containing a heteroatom, said heteroatom being an oxygen atom,
C₃₋₈cycloalkyl substituted with one or more substituents each independently selected from CH₃ and Aryl2,
C₃₋₈cycloalkyl containing a heteroatom and being substituted with one or more substituents each independently selected from the group consisting of CH₃, cyclopropyl, and phenyl, said heteroatom being an oxygen atom,
a 5- to 9-membered fused bicyclic unsaturated or saturated ring, in particular a saturated heterocycle fused with an aromatic ring which may be optionally substituted with OCH₃,
a 5- to 9-membered bridged bicyclic unsaturated or saturated ring, optionally substituted with 1, 2 or 3 CH₃ substituents,
a C₇₋₉spirocycloalkyl, and cubanyl;
   wherein Aryl1 is selected from the group consisting of phenyl, monocyclic heteroaryl, and bicyclic heteroaryl which is in particular an aromatic ring fused to a saturated ring or an aromatic ring fused to another aromatic ring, said Aryl1 being optionally substituted with CH₃;
   wherein Aryl2 is selected from the group consisting of
phenyl optionally substituted with one or more substituents each independently selected from the group consisting of halo, CF₃, CF₂H, CH₂F, C₁₋₄alkyl, C₃₋₆cycloalkyl, CN, CONR¹⁸R¹⁹, OH, OCF₃, OCF₂H, OCH₂F, OC₁₋₄alkyl, OC₃₋₆cycloalkyl, SO₂CH₃, imidazolyl optionally substituted with CH₃, and triazolyl; wherein R¹⁸ and R¹⁹ are independently selected from the group consisting of hydrogen, C₁₋₆alkyl and C₃₋₆cycloalkyl;
monocyclic 5- to 6-membered heteroaryl containing 1, 2 or 3 heteroatoms each independently selected from N, O and S, and being optionally substituted with one or more substituents each independently selected from the group consisting of halo, CF₃, CF₂H, CH₂F, C₁₋₄alkyl, C₃₋₆cycloalkyl, OH, OCF₃, OCF₂H, OCH₂F, OC₁₋₄alkyl, OC₃₋₆cycloalkyl, and phenyl optionally substituted with fluoro;
9- to 10-membered bicyclic heteroaryl which is in particular an aromatic ring fused to a saturated ring or an aromatic ring fused to another aromatic ring, containing 1, 2 or 3 heteroatoms each independently selected from N, S, and O, and being optionally substituted with one or more substituents each independently selected from the group consisting of halo, C₁₋₄alkyl, OC₁₋₄alkyl, and C₃₋₆cycloalkyl;
   or wherein NR′ and R″ together form a saturated cycle or cycle system selected from the group consisting of
a 4- to 7-membered heterocycloalkyl, optionally containing a further heteroatom, said heteroatom being an oxygen, and said ring being optionally substituted with CH₃,
a 4- to 7-membered heterocycloalkyl optionally substituted with one or more substituents each independently selected from C₁₋₆alkyl, phenyl, C₂₋₆alkynyl and C₃-6cycloalkyl,
a C₅₋₁₂-spirocycloalkyl, in particular a C₆₋₈spirocycloalkyl, optionally substituted with CH₃, and
   a C₅₋₆ bridged bicyclic saturated ring system, in particular 2-azabicyclo[2.1.1]hexyl; and all other variables are as defined herein.

In another embodiment, the invention relates to a compound of Formula (I) as defined herein, wherein R⁴ is selected from the group consisting of —OC₁₋₆alkyl, —SC₁₋₆alkyl and NR′R″, wherein

R′ is hydrogen, C₁₋₄alkyl, C₁₋₆alkyl substituted with OH, or C₂₋₃alkenyl; and
R″ is selected from the group consisting of hydrogen, Cycle1, Aryl1, C₂₋₄alkynyl, C₁₋₆alkyl and C₁₋₆alkyl substituted with one or more substituents each independently selected from the group consisting of
fluoro,
OH,
CO₂R¹⁶,
OCONHR¹⁷,
C₃₋₆cycloalkyl,
C₃₋₆cycloalkyl substituted with one or more from among C₁₋₆alkyl,
N-acetyl piperidine,
cubanyl,
benzo[d][1,3]dioxole, and
Aryl2;
   wherein R¹⁶ is hydrogen or C₁₋₆alkyl;
   wherein R¹⁷ is C₁₋₆alkyl;
   wherein Cycle1 is selected from the group consisting of
cyclopropyl, cyclobutyl and cyclopentyl, each of which may be optionally substituted with 1 or 2 substituents, each independently selected from CH₃ and Aryl2,
oxetanyl and tetrahydrofuranyl, each of which may be optionally substituted with one or two substituents, each independently selected from CH₃, cyclopropyl, and phenyl,
a 5- to 9-membered fused bicyclic system in which a saturated heterocycle fused with an aromatic ring which may be optionally substituted with OCH₃, selected from

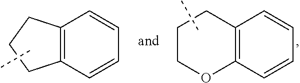

a 5- to 9-membered bridged bicyclic unsaturated or saturated ring selected from the group consisting of bicyclo[1.1.1]pentyl, bicyclo[2.1.0]pentyl, bicyclo[2.1.1]heptane, each of which maybe optionally substituted with 1, 2 or 3 CH₃ substituents;
a C₇₋₉spirocycloalkyl selected from spiro[3.3]heptanyl, and cubanyl;
   wherein Aryl1 is selected from the group consisting of phenyl, and
5- to 6-membered monocyclic heteroaryl, in particular selected from the group consisting of pyrazolyl, oxazolyl, isoxazolyl and triazolyl, each of which is optionally substituted with CH₃,
   wherein Aryl2 is selected from the group consisting of
phenyl optionally substituted with one or more substituents each independently selected from the group consisting of halo, CF₃, CF₂H, CH₂F, C₁₋₄alkyl, C₃₋₆cycloalkyl, CN, CONR¹⁸R¹⁹, OH, OCF₃, OCF₂H, OCH₂F, OC₁₋₄alkyl, OC₃₋₆cycloalkyl, SO₂CH₃, imidazolyl optionally substituted with CH₃, and triazolyl;
   wherein R¹⁸ and R¹⁹ are independently selected from the group consisting of hydrogen, and C₁₋₆alkyl;
monocyclic 5- to 6-membered heteroaryl selected from the group consisting of thiophenyl, pyrazolyl, oxazolyl, isoxazolyl, thiazolyl, triazolyl, pyridyl, pyrimidinyl, pyrazinyl, and pyridazinyl, each of which may be optionally substituted with 1, 2 or 3 substituents each independently selected from the group consisting of halo, CF₃, CF₂H, CH₂F, C₁₋₄alkyl, C₃₋₆cycloalkyl, OH, OCF₃, OCF₂H, OCH₂F, OC₁₋₄alkyl, OC₃₋₆cycloalkyl, and phenyl optionally substituted with fluoro;
9- to 10-membered bicyclic heteroaryl which is in particular an aromatic ring fused to a saturated ring or an aromatic ring fused to another aromatic ring selected from the group consisting of isoquinolinyl, indolyl, 1H-indazolyl, 1H-benzo[d]imidazolyl, and imidazo[1,2-a]pyridinyl, each of which being optionally substituted with one or more substituents each independently selected from the group consisting of halo, C₁₋₄alkyl, OC₁₋₄alkyl, and C₃₋₆cycloalkyl;
   or wherein NR′ and R″ together form a saturated cycle or cycle system selected from the group consisting of
a 4- to 7-membered heterocycloalkyl, optionally containing a further heteroatom, said heteroatom being an oxygen, and said ring being optionally substituted with CH₃,
a 4- to 7-membered heterocycloalkyl optionally substituted with one or more substituents each independently selected from C₁₋₆alkyl, phenyl, C₂₋₆alkynyl and C₃₋₆cycloalkyl,
a C₅₋₁₂-spirocycloalkyl, in particular 2-azaspiro[3.3]heptanyl, optionally substituted with CH₃, and
   a C₅₋₆ bridged bicyclic saturated ring system, in particular 2-azabicyclo[2.1.1]hexyl; and all other variables are as defined herein.

In another embodiment, the invention relates to a compound of Formula (I) as defined herein, wherein R⁴ is selected from the group consisting of —OC₁₋₆alkyl, —SC₁₋₆alkyl and NR′R″, wherein

R′ is hydrogen, C₁₋₄alkyl, or C₁₋₆alkyl substituted with OH; and
R″ is selected from the group consisting of Cycle1, C₁₋₆alkyl and C₁₋₆alkyl substituted with one or more substituents each independently selected from the group consisting of
fluoro,
OH,
C₃₋₆cycloalkyl,
C₃₋₆cycloalkyl substituted with one or more from among C₁₋₆alkyl,
N-acetyl piperidine,
cubanyl,
benzo[d][1,3]dioxole, and
Aryl2;
   wherein Cycle1 is selected from the group consisting of
cyclopropyl, cyclobutyl and cyclopentyl, each of which may be optionally substituted with 1 or 2 substituents, each independently selected from CH₃ and Aryl2,
oxetanyl and tetrahydrofuranyl, each of which may be optionally substituted with one or two substituents, each independently selected from CH₃, cyclopropyl, and phenyl,
a 5- to 9-membered fused bicyclic system in which a saturated heterocycle fused with an aromatic ring which may be optionally substituted with OCH₃, selected from

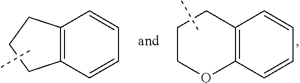

a 5- to 9-membered bridged bicyclic unsaturated or saturated ring selected from the group consisting of bicyclo[1.1.1]pentyl, bicyclo[2.1.0]pentyl, bicyclo[2.1.1]heptane, each of which maybe optionally substituted with 1, 2 or 3 CH₃ substituents;
a C₇₋₉spirocycloalkyl selected from spiro[3.3]heptanyl, and cubanyl;
   wherein Aryl2 is selected from the group consisting of
phenyl optionally substituted with one or more substituents each independently selected from the group consisting of halo, CF₃, CF₂H, CH₂F, C₁₋₄alkyl, C₃₋₆cycloalkyl, CN, CONR¹⁸R¹⁹, OH, OCF₃, OCF₂H, OCH₂F, OC₁₋₄alkyl, OC₃₋₆cycloalkyl, SO₂CH₃, imidazolyl optionally substituted with CH₃, and triazolyl; wherein R¹⁸ and R¹⁹ are independently selected from the group consisting of hydrogen, and C₁₋₆alkyl;
monocyclic 5- to 6-membered heteroaryl selected from the group consisting of thiophenyl, pyrazolyl, oxazolyl, isoxazolyl, thiazolyl, triazolyl, pyridyl, pyrimidinyl, pyrazinyl, and pyridazinyl, each of which may be optionally substituted with 1, 2 or 3 substituents each independently selected from the group consisting of halo, CF₃, CF₂H, CH₂F, C₁₋₄alkyl, C₃₋₆cycloalkyl, OH, OCF₃, OCF₂H, OCH₂F, OC₁₋₄alkyl, OC₃₋₆cycloalkyl, and phenyl optionally substituted with fluoro;
9- to 10-membered bicyclic heteroaryl which is in particular an aromatic ring fused to a saturated ring or an aromatic ring fused to another aromatic ring selected from the group consisting of isoquinolinyl, indolyl, 1H-indazolyl, 1H-benzo[d]imidazolyl, and imidazo[1,2-a]pyridinyl, each of which being optionally substituted with one or more substituents each independently selected from the group consisting of halo, C₁₋₄alkyl, OC₁₋₄alkyl, and C₃₋₆cycloalkyl;
   or wherein NR′ and R″ together form a saturated cycle or cycle system selected from the group consisting of
a 4- to 7-membered heterocycloalkyl, optionally containing a further heteroatom, said heteroatom being an oxygen, and said ring being optionally substituted with CH₃,
a 4- to 7-membered heterocycloalkyl optionally substituted with one or more substituents each independently selected from C₁₋₆alkyl, phenyl, C₂₋₆alkynyl and C₃₋₆cycloalkyl,
a C₅₋₁₂-spirocycloalkyl, in particular 2-azaspiro[3.3]heptanyl, optionally substituted with CH₃, and
   a C₅₋₆ bridged bicyclic saturated ring system, in particular 2-azabicyclo[2.1.1]hexyl; and all other variables are as defined herein.

In another embodiment, the invention relates to a compound of Formula (I) as defined herein, wherein R⁴ is NR′R″, wherein

R′ is hydrogen, C₁₋₄alkyl, or C₁₋₆alkyl substituted with OH; and
R″ is selected from the group consisting of Cycle1, C₁₋₆alkyl and C₁₋₆alkyl substituted with one or more substituents each independently selected from the group consisting of
fluoro,
OH,
C₃₋₆cycloalkyl,
C₃₋₆cycloalkyl substituted with one or more from among C₁₋₆alkyl,
cubanyl,
benzo[d][1,3]dioxole, and
Aryl2;
   wherein Cycle1 is selected from the group consisting of
cyclopropyl, cyclobutyl and cyclopentyl, each of which may be optionally substituted with 1 or 2 substituents, each independently selected from CH₃ and Aryl2, and
oxetanyl and tetrahydrofuranyl, each of which may be optionally substituted with one or two substituents, each independently selected from CH₃, cyclopropyl, and phenyl,
and
cubanyl;
   wherein Aryl2 is selected from the group consisting of
phenyl optionally substituted with one or more substituents each independently selected from the group consisting of halo, CF₃, CF₂H, CH₂F, C₁₋₄alkyl, C₃₋₆cycloalkyl, CN, CONR¹⁸R¹⁹, OH, OCF₃, OCF₂H, OCH₂F, OC₁₋₄alkyl, OC₃₋₆cycloalkyl, SO₂CH₃, imidazolyl optionally substituted with CH₃, and triazolyl;
   wherein R¹⁸ and R¹⁹ are independently selected from the group consisting of hydrogen, and C₁₋₆alkyl;
monocyclic 5- to 6-membered heteroaryl selected from the group consisting of thiophenyl, pyrazolyl, oxazolyl, isoxazolyl, thiazolyl, triazolyl, pyridyl, pyrimidinyl, pyrazinyl, and pyridazinyl, each of which may be optionally substituted with 1, 2 or 3 substituents each independently selected from the group consisting of halo, CF₃, CF₂H, CH₂F, C₁₋₄alkyl, C₃₋₆cycloalkyl, OH, OCF₃, OCF₂H, OCH₂F, OC₁₋₄alkyl, OC₃₋₆cycloalkyl, and phenyl optionally substituted with fluoro;
9- to 10-membered bicyclic heteroaryl which is in particular an aromatic ring fused to a saturated ring or an aromatic ring fused to another aromatic ring selected from the group consisting of isoquinolinyl, indolyl, 1H-indazolyl, 1H-benzo[d]-imidazolyl, and imidazo[1,2-a]pyridinyl, each of which being optionally substituted with one or more substituents each independently selected from the group consisting of halo, C₁₋₄alkyl, OC₁₋₄alkyl, and C₃₋₆cycloalkyl;
   or wherein NR′ and R″ together form a saturated cycle or cycle system selected from the group consisting of
a 4- to 7-membered heterocycloalkyl, optionally containing a further heteroatom, said heteroatom being an oxygen, and said ring being optionally substituted with CH₃,
a 4- to 7-membered heterocycloalkyl optionally substituted with one or more substituents each independently selected from C₁₋₆alkyl, phenyl, C₂₋₆alkynyl and C₃₋₆cycloalkyl,
a C₅₋₁₂-spirocycloalkyl, in particular 2-azaspiro[3.3]heptanyl, optionally substituted with CH₃, and
   a C₅₋₆ bridged bicyclic saturated ring system, in particular 2-azabicyclo[2.1.1]hexyl; and all other variables are as defined herein.

In another embodiment, the invention relates to a compound of Formula (I) as defined herein, wherein R⁴ is NR′R″, wherein

R′ is hydrogen; and
R″ is selected from the group consisting of Cycle1, C₁₋₆alkyl and C₁₋₆alkyl substituted with one or more substituents each independently selected from the group consisting of
fluoro,
C₃₋₆cycloalkyl,
C₃₋₆cycloalkyl substituted with one or more from among C₁₋₆alkyl,
cubanyl,
and
Aryl2;
   wherein Cycle1 is selected from the group consisting of
cyclopropyl, cyclobutyl and cyclopentyl, each of which may be optionally substituted with 1 or 2 substituents, each independently selected from CH₃ and Aryl2, and
oxetanyl and tetrahydrofuranyl, each of which may be optionally substituted with one or two substituents, each independently selected from CH₃, cyclopropyl, and phenyl,
   wherein Aryl2 is selected from the group consisting of
phenyl optionally substituted with one or more substituents each independently selected from the group consisting of halo, CF₃, CF₂H, CH₂F, C₁₋₄alkyl, C₃₋₆cycloalkyl, CN, CONR¹⁸R¹⁹, OH, OCF₃, OCF₂H, OCH₂F, OC₁₋₄alkyl, OC₃₋₆cycloalkyl, SO₂CH₃, imidazolyl optionally substituted with CH₃, and triazolyl;
   wherein R¹⁸ and R¹⁹ are independently selected from the group consisting of hydrogen, and C₁₋₆alkyl;
monocyclic 5- to 6-membered heteroaryl selected from the group consisting of thiophenyl, pyrazolyl, oxazolyl, isoxazolyl, thiazolyl, triazolyl, pyridyl, pyrimidinyl, pyrazinyl, and pyridazinyl, each of which may be optionally substituted with 1, 2 or 3 substituents each independently selected from the group consisting of halo, CF₃, CF₂H, CH₂F, C₁₋₄alkyl, C₃₋₆cycloalkyl, OH, OCF₃, OCF₂H, OCH₂F, OC₁₋₄alkyl, OC₃₋₆cycloalkyl, and phenyl optionally substituted with fluoro;
   or wherein NR′ and R″ together form a saturated cycle or cycle system selected from the group consisting of
a 4- to 7-membered heterocycloalkyl, optionally containing a further heteroatom, said heteroatom being an oxygen, and said ring being optionally substituted with CH₃,
a 4- to 7-membered heterocycloalkyl optionally substituted with one or more substituents each independently selected from C₁₋₆alkyl, phenyl, C₂₋₆alkynyl and C₃₋₆cycloalkyl,
a C₅₋₁₂-spirocycloalkyl, in particular 2-azaspiro[3.3]heptanyl, optionally substituted with CH₃, and
a C₅₋₆ bridged bicyclic saturated ring system, in particular 2-azabicyclo[2.1.1]hexyl;
   and all other variables are as defined herein.

In another embodiment, the invention relates to a compound of Formula (I) as defined herein, wherein R⁴ is NR′R″, wherein

R′ is hydrogen; and
R″ is selected from the group consisting of Cycle1, C₁₋₆alkyl and C₁₋₆alkyl substituted with one or more substituents each independently selected from the group consisting of
fluoro,
C₃₋₆cycloalkyl,
C₃₋₆cycloalkyl substituted with one or more from among C₁₋₆alkyl,
cubanyl,
and
Aryl2;
   wherein Cycle1 is selected from the group consisting of
cyclopropyl, cyclobutyl and cyclopentyl, each of which may be optionally substituted with 1 or 2 substituents, each independently selected from CH₃ and Aryl2, and
oxetanyl and tetrahydrofuranyl, each of which may be optionally substituted with one or two substituents, each independently selected from CH₃, cyclopropyl, and phenyl,
   wherein Aryl2 is phenyl optionally substituted with one or more substituents each independently selected from the group consisting of halo, CF₃, CF₂H, CH₂F, C₁₋₄alkyl, C₃₋₆cycloalkyl, CN, CONR¹⁸R¹⁹, OH, OCF₃, OCF₂H, OCH₂F, OC₁₋₄alkyl, OC₃₋₆cycloalkyl, and SO₂CH₃;
   wherein R¹⁸ and R¹⁹ are independently selected from the group consisting of hydrogen, and C₁₋₆alkyl;
   or wherein NR′ and R″ together form a saturated cycle or cycle system selected from the group consisting of
a 4- to 7-membered heterocycloalkyl, optionally containing a further heteroatom, said heteroatom being an oxygen, and said ring being optionally substituted with CH₃,
a 4- to 7-membered heterocycloalkyl optionally substituted with one or more substituents each independently selected from the group consisting of C₁₋₆alkyl, phenyl, C₂₋₆alkynyl and C₃₋₆cycloalkyl,
a C₅₋₁₂-spirocycloalkyl, in particular 2-azaspiro[3.3]heptanyl, optionally substituted with CH₃, and
a C₅₋₆ bridged bicyclic saturated ring system, in particular 2-azabicyclo[2.1.1]hexyl;
   and all other variables are as defined herein.

In another embodiment, the invention relates to a compound of Formula (I) as defined herein, wherein R⁴ is NR′R″, wherein

R′ is hydrogen; and
R″ is selected from the group consisting of Cycle1, C₁₋₆alkyl and C₁₋₆alkyl substituted with one or more substituents each independently selected from the group consisting of
fluoro,
C₃₋₆cycloalkyl,
C₃₋₆cycloalkyl substituted with one or more from among C₁₋₆alkyl,
cubanyl,
and
Aryl2;
   wherein Cycle1 is selected from the group consisting of
cyclopropyl, cyclobutyl and cyclopentyl, each of which may be optionally substituted with 1 or 2 substituents, each independently selected from CH₃ and Aryl2, and
oxetanyl and tetrahydrofuranyl, each of which may be optionally substituted with one or two substituents, each independently selected from CH₃, cyclopropyl, and phenyl,
   wherein Aryl2 is a monocyclic 5- to 6-membered heteroaryl selected from the group consisting of thiophenyl, pyrazolyl, oxazolyl, isoxazolyl, thiazolyl, triazolyl, pyridyl, pyrimidinyl, pyrazinyl, and pyridazinyl, each of which may be optionally substituted with 1, 2 or 3 substituents each independently selected from the group consisting of halo, CF₃, CF₂H, CH₂F, C₁₋₄alkyl, C₃₋₆cycloalkyl, OH, OCF₃, OCF₂H, OCH₂F, OC₁₋₄alkyl, OC₃₋₆cycloalkyl, and phenyl optionally substituted with fluoro;
   or wherein NR′ and R″ together form a saturated cycle or cycle system selected from the group consisting of
a 4- to 7-membered heterocycloalkyl, optionally containing a further heteroatom, said heteroatom being an oxygen, and said ring being optionally substituted with CH₃,
a 4- to 7-membered heterocycloalkyl optionally substituted with one or more substituents each independently selected from the group consisting of C₁₋₆alkyl, phenyl, C₂₋₆alkynyl and C₃₋₆cycloalkyl,
a C₅₋₁₂-spirocycloalkyl, in particular 2-azaspiro[3.3]heptanyl, optionally substituted with CH₃, and
a C₅₋₆ bridged bicyclic saturated ring system, in particular 2-azabicyclo[2.1.1]hexyl;
   and all other variables are as defined herein.

In another embodiment, the invention relates to a compound of Formula (I) as defined herein, wherein

R⁵ is selected from the group consisting of hydrogen, C₁₋₆alkyl, C₂₋₃alkenyl, Cycle2 and Aryl3;
wherein C₁₋₆alkyl is optionally substituted with one or more substituents each independently selected from the group consisting of phenyl, methoxyphenyl, OC₁₋₆alkyl, NHSO₂CH₃, and C₃₋₆cycloalkyl;
wherein Cycle2 is selected from the group consisting of
C₃₋₆cycloalkyl,
C₃₋₆cycloalkyl containing SO₂ or a heteroatom, the heteroatom being selected from the group consisting of oxygen and nitrogen,
C₃₋₆cycloalkyl substituted with CO₂R^{20a}, CONHR^{20b} or SO₂C₁₋₆alkyl,
C₃₋₆cycloalkyl containing SO₂ or a heteroatom and being substituted with CO₂R^{20a}, CONHR^{20b} or SO₂C₁₋₆alkyl, the heteroatom being selected from the group consisting of oxygen and nitrogen,
a 5-membered bridged bicyclic saturated ring substituted with CO₂C₁₋₆alkyl or CONHR^{20b},
cubanyl optionally substituted with CO₂C₁₋₆alkyl or CONHR^{20b},
isoindoline-1-one, and
indoline-2-one;
   wherein R^{20a} is hydrogen or C₁₋₆alkyl;
   wherein R^{20b} is C₁₋₆alkyl or C₃₋₆cycloalkyl;
   wherein Aryl3 is selected from the group consisting of phenyl, monocyclic heteroaryl, and bicyclic heteroaryl, said Aryl3 being optionally substituted with one or more substituents each independently selected from the group consisting of halo, C₁₋₆alkyl, CF₃, CF₂H, CH₂F, CN, OC₁₋₆alkyl, OCF₃, OCF₂H, OCH₂F, OC₃₋₆cycloalkyl, SO₂R²¹, SO₂NHR²², CO₂R²³, COR²⁴, CONR²⁵R²⁶, NHR²⁷, NHCOR²⁸, Cycle3 and Aryl4;
   wherein R²¹ is C₁₋₆alkyl or C₃₋₆cycloalkyl;
   wherein R²² is C₁₋₆alkyl or pyridine;
   wherein R²³ is hydrogen or C₁₋₆alkyl;
   wherein R²⁴ is selected from the group consisting of C₁₋₆alkyl, C₅₋₆heterocycle and C₅₋₆heterocycle substituted with CH₃;
   wherein R²⁵ is hydrogen or CH₃;
   wherein R²⁶ is selected from the group consisting of hydrogen,
C₁₋₆alkyl,
C₁₋₆alkyl substituted with one or more substituents each independently selected from the group consisting of OH, OCH₃, NH₂, CO₂H, C₃₋₆heterocycloalkyl and C₃₋₆heterocycloalkyl substituted with CH₃,
C₃₋₄cycloalkyl;
C₃₋₄cycloalkyl containing a heteroatom, said heteroatom being an oxygen atom;
C₃₋₄cycloalkyl substituted with CO₂H; and
wherein R²⁷ is C₁₋₆alkyl;
   wherein R²⁸ is C₁₋₆alkyl or C₃₋₆cycloalkyl;
   wherein Cycle3 is selected from the group consisting of
C₃₋₆cycloalkyl,
C₃₋₆heterocycloalkyl,
C₃₋₆heterocycloalkyl substituted with one or more substituents each independently selected from the group consisting of OH, CH₂OH, CO₂R²⁹, NHCH₃ or NHCO₂t-Bu; and
imidazolidin-4-one substituted with CH₃;
   wherein R²⁹ is hydrogen or C₁₋₆alkyl; and
   wherein Aryl4 is selected from the group consisting of monocyclic heteroaryl and bicyclic heteroaryl, said monocyclic or bicyclic heteroaryl being optionally substituted with one or two substituents each independently selected from the group consisting of halogens, CF₃, CH₂F, C₁₋₆alkyl, C₃₋₆cycloalkyl, OCF₃, OCH₂F, OC₁₋₆alkyl, OC₃₋₆cycloalkyl, CO₂R³⁰, SO₂CH₃, and morpholine;
   wherein R³⁰ is hydrogen or C₁₋₆alkyl;
   and all other variables are as defined herein.

In another embodiment, the invention relates to a compound of Formula (I) as defined herein, wherein

R⁵ is selected from the group consisting of hydrogen, C₁₋₆alkyl, Cycle2 and Aryl3;
wherein C₁₋₆alkyl is optionally substituted with one or more substituents each independently selected from the group consisting of phenyl, methoxyphenyl, OC₁₋₆alkyl, NHSO₂CH₃, and C₃₋₆cycloalkyl;
wherein Cycle2 is selected from the group consisting of
C₃₋₆cycloalkyl,
C₃₋₆cycloalkyl containing SO₂ or a heteroatom, the heteroatom being selected from the group consisting of oxygen and nitrogen,
C₃₋₆cycloalkyl substituted with CONHR^{20b} or SO₂C₁₋₆alkyl,
C₃₋₆cycloalkyl containing SO₂ or a heteroatom and being substituted with CONHR^{20b} or SO₂C₁₋₆alkyl, the heteroatom being selected from the group consisting of oxygen and nitrogen,
a 5-membered bridged bicyclic saturated ring, in particular bicyclo[1.1.1]pentanyl or bicyclo[2.1.0]pentanyl, substituted with CONHR^{20b}, and
cubanyl optionally substituted with CONHR^{20b};
   wherein R^{20b} is C₁₋₆alkyl or C₃₋₆cycloalkyl;
   wherein Aryl3 is selected from the group consisting of
phenyl optionally substituted with one or more substituents each independently selected from the group consisting of halo, C₁₋₆alkyl, CF₃, CF₂H, CH₂F, CN, OC₁₋₆alkyl, OCF₃, OCF₂H, OCH₂F, OC₃₋₆cycloalkyl, SO₂R²¹, SO₂NHR²², COR²⁴, CONR²⁵R²⁶, NHR²⁷, NHCOR²⁸, Cycle3 and Aryl4;
5- to 6-membered monocyclic heteroaryl selected from the group consisting of pyrazolyl, imidazolyl, oxazolyl, isoxazolyl, thiazolyl, triazolyl, pyridyl, pyridazinyl, pyrimidinyl, and pyrazinyl, each of which may be optionally substituted with one or more substituents each independently selected from the group consisting of halo, C₁₋₆alkyl, OC₁₋₆alkyl, SO₂R²¹, CONR²⁵R²⁶, and NHR²⁷; and bicyclic heteroaryl selected from the group consisting of 1H-indolyl, 1H-indazolyl, benzo[d]oxazolyl, and benzo[d]isoxazolyl, each of which may be optionally substituted with one or more substituents each independently selected from the group consisting of C₁₋₆alkyl, CONR²⁵R²⁶, and NHR²⁷;
   wherein R²¹ is C₁₋₆alkyl or C₃₋₆cycloalkyl;
   wherein R²² is C₁₋₆alkyl or pyridine;
   wherein R²⁴ is selected from the group consisting of C₁₋₆alkyl, and morpholinyl or piperazinyl each of which may be optionally substituted with CH₃;
   wherein R²⁵ is hydrogen or CH₃;
   wherein R²⁶ is selected from the group consisting of
hydrogen,
C₁₋₆alkyl,
C₁₋₆alkyl substituted with one or more substituents each independently selected from the group consisting of OH, OCH₃, NH₂, CO₂H, and morpholinyl or piperazinyl each of which may be optionally substituted with CH₃,
C₃₋₄cycloalkyl; and
C₃₋₄cycloalkyl containing a heteroatom, said heteroatom being an oxygen atom;
wherein R²⁷ is C₁₋₆alkyl;
   wherein R²⁸ is C₁₋₆alkyl or C₃₋₆cycloalkyl;
   wherein Cycle3 is selected from the group consisting of
cyclopropyl,
C₃₋₆heterocycloalkyl, in particular pyrrolidinyl or morpholinyl, substituted with one or more substituents each independently selected from the group consisting of OH, CH₂OH, CO₂R²⁹, NHCH₃ or NHCO₂t-Bu; and
imidazolidin-4-one substituted with CH₃;
   wherein R²⁹ is hydrogen or C₁₋₆alkyl; and
   wherein Aryl4 is a monocyclic heteroaryl selected from the group consisting of furanyl, pyrazolyl, imidazolyl, oxazolyl, isoxazolyl, thiazolyl, isothiazolyl, triazolyl, tetrazolyl, oxadiazolyl, thiadiazolyl, pyridyl, pyridazinyl, pyrimidinyl, and pyrazinyl, each of which may be optionally substituted with one or two substituents each independently selected from the group consisting of halogens, CF₃, CH₂F, C₁₋₆alkyl, C₃₋₆cycloalkyl, CO₂R³⁰, SO₂CH₃, and morpholine;
   wherein R³⁰ is hydrogen or C₁₋₆alkyl;
   and all other variables are as defined herein.

In another embodiment, the invention relates to a compound of Formula (I) as defined herein, wherein

R⁵ is selected from the group consisting of Cycle2 and Aryl3;
wherein Cycle2 is selected from the group consisting of
C₃₋₆cycloalkyl,
C₃₋₆cycloalkyl containing SO₂ or a heteroatom, the heteroatom being selected from the group consisting of oxygen and nitrogen,
C₃₋₆cycloalkyl substituted with CONHR^{20b} or SO₂C₁₋₆alkyl,
C₃₋₆cycloalkyl containing SO₂ or a heteroatom and being substituted with CONHR^{20b} or SO₂C₁₋₆alkyl, the heteroatom being selected from the group consisting of oxygen and nitrogen,
a 5-membered bridged bicyclic saturated ring, in particular bicyclo[1.1.1]pentanyl or bicyclo[2.1.0]pentanyl, substituted with CONHR^{20b}, and cubanyl optionally substituted with CONHR^{20b};
   wherein R^{20b} is C₁₋₆alkyl or C₃₋₆cycloalkyl;
   wherein Aryl3 is selected from the group consisting of
phenyl optionally substituted with one or more substituents each independently selected from the group consisting of halo, C₁₋₆alkyl, CF₃, CF₂H, CH₂F, CN, OC₁₋₆alkyl, OCF₃, OCF₂H, OCH₂F, OC₃₋₆cycloalkyl, SO₂R²¹, SO₂NHR²², COR²⁴, CONR²⁵R²⁶, NHR²⁷, NHCOR²⁸, Cycle3 and Aryl4;
5- to 6-membered monocyclic heteroaryl selected from the group consisting of pyrazolyl, imidazolyl, oxazolyl, isoxazolyl, thiazolyl, triazolyl, pyridyl, pyridazinyl, pyrimidinyl, and pyrazinyl, each of which may be optionally substituted with one or more substituents each independently selected from the group consisting of halo, C₁₋₆alkyl, OC₁₋₆alkyl, SO₂R²¹, CONR²⁵R²⁶, and NHR²⁷; and bicyclic heteroaryl selected from the group consisting of 1H-indolyl, 1H-indazolyl, benzo[d]oxazolyl, and benzo[d]isoxazolyl, each of which may be optionally substituted with one or more substituents each independently selected from the group consisting of C₁₋₆alkyl, CONR²⁵R²⁶, and NHR²⁷;
   wherein R²¹ is C₁₋₆alkyl or C₃₋₆cycloalkyl;
   wherein R²² is C₁₋₆alkyl or pyridine;
   wherein R²⁴ is selected from the group consisting of C₁₋₆alkyl, and morpholinyl or piperazinyl each of which may be optionally substituted with CH₃;
   wherein R²⁵ is hydrogen or CH₃;
   wherein R²⁶ is selected from the group consisting of
hydrogen,
C₁₋₆alkyl,
C₁₋₆alkyl substituted with one or more substituents each independently selected from the group consisting of OH, OCH₃, NH₂, CO₂H, and morpholinyl or piperazinyl each of which may be optionally substituted with CH₃,
C₃₋₄cycloalkyl; and
C₃₋₄cycloalkyl containing a heteroatom, said heteroatom being an oxygen atom;
wherein R²⁷ is C₁₋₆alkyl;
   wherein R²⁸ is C₆alkyl or C₃₋₆cycloalkyl;
   wherein Cycle3 is selected from the group consisting of cyclopropyl,
C₃₋₆heterocycloalkyl, in particular pyrrolidinyl or morpholinyl, substituted with one or more substituents each independently selected from the group consisting of OH, CH₂OH, CO₂R²⁹, NHCH₃ or NHCO₂t-Bu; and
imidazolidin-4-one substituted with CH₃;
   wherein R²⁹ is hydrogen or C₁₋₆alkyl; and
   wherein Aryl4 is a monocyclic heteroaryl selected from the group consisting of furanyl, pyrazolyl, imidazolyl, oxazolyl, isoxazolyl, thiazolyl, isothiazolyl, triazolyl, tetrazolyl, oxadiazolyl, thiadiazolyl, pyridyl, pyridazinyl, pyrimidinyl, and pyrazinyl, each of which may be optionally substituted with one or two substituents each independently selected from the group consisting of halogens, CF₃, CH₂F, C₁₋₆alkyl, C₃₋₆cycloalkyl, CO₂R³⁰, SO₂CH₃, and morpholine;
   wherein R³⁰ is hydrogen or C₁₋₆alkyl;
   and all other variables are as defined herein.

In another embodiment, the invention relates to a compound of Formula (I) as defined herein, wherein

### R⁵ is Aryl3;

wherein Aryl3 is selected from the group consisting of
phenyl optionally substituted with one or more substituents each independently selected from the group consisting of halo, C₁₋₆alkyl, CF₃, CF₂H, CH₂F, CN, OC₁₋₆alkyl, OCF₃, OCF₂H, OCH₂F, OC₃₋₆cycloalkyl, SO₂R²¹, SO₂NHR²², COR²⁴, CONR²⁵R²⁶, NHR²⁷, NHCOR²⁸, Cycle3 and Aryl4;
5- to 6-membered monocyclic heteroaryl selected from the group consisting of pyrazolyl, imidazolyl, oxazolyl, isoxazolyl, thiazolyl, triazolyl, pyridyl, pyridazinyl, pyrimidinyl, and pyrazinyl, each of which may be optionally substituted with one or more substituents each independently selected from the group consisting of halo, C₁₋₆alkyl, OC₁₋₆alkyl, SO₂R²¹, CONR²⁵R²⁶, and NHR²⁷; and bicyclic heteroaryl selected from the group consisting of 1H-indolyl, 1H-indazolyl, benzo[d]oxazolyl, and benzo[d]isoxazolyl, each of which may be optionally substituted with one or more substituents each independently selected from the group consisting of C₁₋₆alkyl, CONR²⁵R²⁶, and NHR²⁷;
   wherein R²¹ is C₁₋₆alkyl or C₃₋₆cycloalkyl;
   wherein R²² is C₁₋₆alkyl or pyridine;
   wherein R²⁴ is selected from the group consisting of C₁₋₆alkyl, and morpholinyl or piperazinyl each of which may be optionally substituted with CH₃;
   wherein R²⁵ is hydrogen or CH₃;
   wherein R²⁶ is selected from the group consisting of
hydrogen,
C₁₋₆alkyl,
C₁₋₆alkyl substituted with one or more substituents each independently selected from the group consisting of OH, OCH₃, NH₂, CO₂H, and morpholinyl or piperazinyl each of which may be optionally substituted with CH₃,
C₃₋₄cycloalkyl; and
C₃₋₄cycloalkyl containing a heteroatom, said heteroatom being an oxygen atom;
wherein R²⁷ is C₁₋₆alkyl;
   wherein R²⁸ is C₁₋₆alkyl or C₃₋₆cycloalkyl;
   wherein Cycle3 is selected from the group consisting of
cyclopropyl,
C₃₋₆heterocycloalkyl, in particular pyrrolidinyl or morpholinyl, substituted with one or more substituents each independently selected from the group consisting of OH, CH₂OH, CO₂R²⁹, NHCH₃ or NHCO₂t-Bu; and
imidazolidin-4-one substituted with CH₃;
   wherein R²⁹ is hydrogen or C₁₋₆alkyl; and
   wherein Aryl4 is a monocyclic heteroaryl selected from the group consisting of furanyl, pyrazolyl, imidazolyl, oxazolyl, isoxazolyl, thiazolyl, isothiazolyl, triazolyl, tetrazolyl, oxadiazolyl, thiadiazolyl, pyridyl, pyridazinyl, pyrimidinyl, and pyrazinyl, each of which may be optionally substituted with one or two substituents each independently selected from the group consisting of halogens, CF₃, CH₂F, C₁₋₆alkyl, C₃₋₆cycloalkyl, CO₂R³⁰, SO₂CH₃, and morpholine;
   wherein R³⁰ is hydrogen or C₁₋₆alkyl;
   and all other variables are as defined herein.

In another embodiment, the invention relates to a compound of Formula (I) as defined herein, wherein

### R⁵ is Aryl3;

wherein Aryl3 is selected from the group consisting of
phenyl and 5- to 6-membered monocyclic heteroaryl selected from the group consisting of pyrazolyl, imidazolyl, oxazolyl, isoxazolyl, thiazolyl, triazolyl, pyridyl, pyridazinyl, pyrimidinyl, and pyrazinyl, each of which may be optionally substituted with one or more substituents each independently selected from the group consisting of halo, C₁₋₆alkyl, OC₁₋₆alkyl, SO₂R²¹, CONR²⁵R²⁶, and NHR²⁷;
   wherein R²¹ is C₁₋₆alkyl or C₃₋₆cycloalkyl;
   wherein R²⁵ is hydrogen or CH₃;
   wherein R²⁶ is selected from the group consisting of
hydrogen,
C₁₋₆alkyl,
C₁₋₆alkyl substituted with one or more substituents each independently selected from the group consisting of OH, OCH₃, NH₂, CO₂H, and morpholinyl or piperazinyl each of which may be optionally substituted with CH₃,
C₃₋₄cycloalkyl; and
C₃₋₄cycloalkyl containing a heteroatom, said heteroatom being an oxygen atom;
wherein R²⁷ is C₁₋₆alkyl;
   and all other variables are as defined herein.

In another embodiment, the invention relates to a compound of Formula (I) as defined herein, wherein

### R⁵ is Aryl3;

wherein Aryl3 is selected from the group consisting of
phenyl optionally substituted with one or more substituents each independently selected from the group consisting of halo, C₁₋₆alkyl, CF₃, CF₂H, CH₂F, CN, OC₁₋₆alkyl, OCF₃, OCF₂H, OCH₂F, OC₃₋₆cycloalkyl, SO₂R²¹, SO₂NHR²², COR²⁴, CONR²⁵R²⁶, NHR²⁷, NHCOR²⁸, Cycle3 and Aryl4;
   wherein R²¹ is C₁₋₆alkyl or C₃₋₆cycloalkyl;
   wherein R²² is C₁₋₆alkyl or pyridine;
   wherein R²⁴ is selected from the group consisting of C₁₋₆alkyl, and morpholinyl or piperazinyl each of which may be optionally substituted with CH₃;
   wherein R²⁵ is hydrogen or CH₃;
   wherein R²⁶ is selected from the group consisting of
hydrogen,
C₁₋₆alkyl,
C₁₋₆alkyl substituted with one or more substituents each independently selected from the group consisting of OH, OCH₃, NH₂, CO₂H, and morpholinyl or piperazinyl each of which may be optionally substituted with CH₃,
C₃₋₄cycloalkyl; and
C₃₋₄cycloalkyl containing a heteroatom, said heteroatom being an oxygen atom;
wherein R²⁷ is C₆alkyl;
   wherein R²⁸ is C₁₋₆alkyl or C₃₋₆cycloalkyl;
   wherein Cycle3 is selected from the group consisting of
cyclopropyl,
C₃₋₆heterocycloalkyl, in particular pyrrolidinyl or morpholinyl, substituted with one or more substituents each independently selected from the group consisting of OH, CH₂OH, CO₂R²⁹, NHCH₃ or NHCO₂t-Bu; and
imidazolidin-4-one substituted with CH₃;
   wherein R²⁹ is hydrogen or C₁₋₆alkyl; and
   wherein Aryl4 is a monocyclic heteroaryl selected from the group consisting of furanyl, pyrazolyl, imidazolyl, oxazolyl, isoxazolyl, thiazolyl, isothiazolyl, triazolyl, tetrazolyl, oxadiazolyl, thiadiazolyl, pyridyl, pyridazinyl, pyrimidinyl, and pyrazinyl, each of which may be optionally substituted with one or two substituents each independently selected from the group consisting of halogens, CF₃, CH₂F, C₁₋₆alkyl, C₃₋₆cycloalkyl, CO₂R³⁰, SO₂CH₃, and morpholine;
   wherein R³⁰ is hydrogen or C₁₋₆alkyl;
   and all other variables are as defined herein.

In another embodiment, the invention relates to a compound of Formula (I) as defined herein, wherein

### R⁵ is Aryl3;

wherein Aryl3 is selected from the group consisting of
phenyl substituted with one or more substituents each independently selected from the group consisting of Cycle3 and Aryl4;
   wherein Cycle3 is selected from the group consisting of
cyclopropyl,
C₃₋₆heterocycloalkyl, in particular pyrrolidinyl or morpholinyl, substituted with one or more substituents each independently selected from the group consisting of OH, CH₂OH, CO₂R²⁹, NHCH₃ or NHCO₂t-Bu; and
imidazolidin-4-one substituted with CH₃;
   wherein R²⁹ is hydrogen or C₁₋₆alkyl; and
   wherein Aryl4 is a monocyclic heteroaryl selected from the group consisting of furanyl, pyrazolyl, imidazolyl, oxazolyl, isoxazolyl, thiazolyl, isothiazolyl, triazolyl, tetrazolyl, oxadiazolyl, thiadiazolyl, pyridyl, pyridazinyl, pyrimidinyl, and pyrazinyl, each of which may be optionally substituted with one or two substituents each independently selected from the group consisting of halogens, CF₃, CH₂F, C₁₋₆alkyl, C₃₋₆cycloalkyl, CO₂R³⁰, SO₂CH₃, and morpholine;
   wherein R³⁰ is hydrogen or C₁₋₆alkyl;
   and all other variables are as defined herein.

In another embodiment, the invention relates to a compound of Formula (I) as defined herein, wherein

R⁵ is selected from the group consisting of Cycle2 and Aryl3;
wherein Cycle2 is selected from the group consisting of
C₃₋₆cycloalkyl substituted with CONHR^{20b},
C₃₋₆cycloalkyl containing SO₂ or a heteroatom and being substituted with CONHR^{20b}, the heteroatom being selected from the group consisting of oxygen and nitrogen,
a 5-membered bridged bicyclic saturated ring, in particular bicyclo[1.1.1]pentanyl or bicyclo[2.1.0]pentanyl, substituted with CONHR^{20b}, and cubanyl optionally substituted with CONHR^{20b};
   wherein R^{20b} is C₁₋₆alkyl or C₃₋₆cycloalkyl;
   wherein Aryl3 is selected from the group consisting of
phenyl substituted with one or more substituents each independently selected from the group consisting of C₁₋₆alkyl, CONR²⁵R²⁶, and NHR²⁷;
5- to 6-membered monocyclic heteroaryl selected from the group consisting of pyrazolyl, imidazolyl, oxazolyl, isoxazolyl, thiazolyl, triazolyl, pyridyl, pyridazinyl, pyrimidinyl, and pyrazinyl, each of which being substituted with one or more substituents each independently selected from the group consisting of C₁₋₆alkyl, CONR²⁵R²⁶, and NHR²⁷; and
   bicyclic heteroaryl selected from the group consisting of 1H-indolyl, 1H-indazolyl, benzo[d]oxazolyl, and benzo[d]isoxazolyl, each of which may be optionally substituted with one or more substituents each independently selected from the group consisting of C₁₋₆alkyl, CONR²⁵R²⁶, and NHR²⁷;
   wherein R²⁵ is hydrogen or CH₃;
   wherein R²⁶ is selected from the group consisting of
hydrogen,
C₁₋₆alkyl,
C₁₋₆alkyl substituted with one or more substituents each independently selected from the group consisting of OH, OCH₃, NH₂, CO₂H, and morpholinyl or piperazinyl each of which may be optionally substituted with CH₃,
C₃₋₄cycloalkyl; and
C₃₋₄cycloalkyl containing a heteroatom, said heteroatom being an oxygen atom;
wherein R²⁷ is C₁₋₆alkyl;
   and all other variables are as defined herein.

In another embodiment, the invention relates to a compound of Formula (I) as defined herein, wherein

R⁵ is selected from the group consisting of Cycle2 and Aryl3;
wherein Cycle2 is selected from the group consisting of
C₃₋₆cycloalkyl substituted with CONHR^{20b},
C₃₋₆cycloalkyl containing SO₂ or a heteroatom and being substituted with CONHR^{20b}, the heteroatom being selected from the group consisting of oxygen and nitrogen,
a 5-membered bridged bicyclic saturated ring, in particular bicyclo[1.1.1]pentanyl or bicyclo[2.1.0]pentanyl, substituted with CONHR^{20b}, and cubanyl optionally substituted with CONHR^{20b};
   wherein R^{20b} is C₁₋₆alkyl or C₃₋₆cycloalkyl;
   wherein Aryl3 is selected from the group consisting of
phenyl or a 5- to 6-membered monocyclic heteroaryl selected from the group consisting of pyrazolyl, imidazolyl, oxazolyl, isoxazolyl, thiazolyl, triazolyl, pyridyl, pyridazinyl, pyrimidinyl, and pyrazinyl, each of which being substituted with one or more substituents each independently selected from the group consisting of C₁₋₆alkyl, CONR²⁵R²⁶, and NHR²⁷; and
   wherein R²⁵ is hydrogen or CH₃;
   wherein R²⁶ is selected from the group consisting of hydrogen,
C₁₋₆alkyl,
C₁₋₆alkyl substituted with one or more substituents each independently selected from the group consisting of OH, OCH₃, NH₂, CO₂H, and morpholinyl or piperazinyl each of which may be optionally substituted with CH₃,
C₃₋₄cycloalkyl; and
C₃₋₄cycloalkyl containing a heteroatom, said heteroatom being an oxygen atom; and
   wherein R²⁷ is C₁₋₆alkyl;
   and all other variables are as defined herein.

In another embodiment, the invention relates to a compound of Formula (I) as defined herein, wherein

R⁵ is selected from the group consisting of Cycle2 and Aryl3;
wherein Cycle2 is selected from the group consisting of
C₃₋₆cycloalkyl and C₃₋₆cycloalkyl containing SO₂ or a heteroatom selected from the group consisting of oxygen and nitrogen, each of which being substituted with CONHR^{20b}, wherein R^{20b} is C₁₋₆alkyl or C₃₋₆cycloalkyl;
   wherein Aryl3 is selected from the group consisting of
phenyl or a 5- to 6-membered monocyclic heteroaryl selected from the group consisting of pyrazolyl, imidazolyl, oxazolyl, isoxazolyl, thiazolyl, triazolyl, pyridyl, pyridazinyl, pyrimidinyl, and pyrazinyl, each of which being substituted with one or more substituents each independently selected from the group consisting of C₁₋₆alkyl, CONR²⁵R²⁶, and NHR²⁷; and
   wherein R²⁵ is hydrogen or CH₃;
   wherein R²⁶ is selected from the group consisting of
C₁₋₆alkyl,
C₁₋₆alkyl substituted with one or more substituents each independently selected from the group consisting of OH, OCH₃, NH₂, CO₂H, and morpholinyl or piperazinyl each of which may be optionally substituted with CH₃,
C₃₋₄cycloalkyl; and
C₃₋₄cycloalkyl containing a heteroatom, said heteroatom being an oxygen atom; and wherein R²⁷ is C₁₋₆alkyl;
   and all other variables are as defined herein.

In another embodiment, the invention relates to a compound of Formula (I) as defined herein, wherein

R⁵ is selected from the group consisting of Cycle2 and Aryl3;
wherein Cycle2 is selected from the group consisting of
C₃₋₆cycloalkyl and C₃₋₆cycloalkyl containing SO₂ or a heteroatom selected from the group consisting of oxygen and nitrogen, each of which being substituted with CONHR^{20b}, wherein R^{20b} is C₁₋₆alkyl or C₃₋₆cycloalkyl;
   wherein Aryl3 is selected from the group consisting of
phenyl or a 5- to 6-membered monocyclic heteroaryl selected from the group consisting of pyrazolyl, imidazolyl, oxazolyl, isoxazolyl, thiazolyl, triazolyl, pyridyl, pyridazinyl, pyrimidinyl, and pyrazinyl, each of which being substituted with one or more substituents each independently selected from the group consisting of C₁₋₆alkyl, CONR²⁵R²⁶, and NHR²⁷; and
   wherein R²⁵ is hydrogen or CH₃;
   wherein R²⁶ is selected from the group consisting of
C₁₋₆alkyl,
C₃₋₄cycloalkyl, and
C₃₋₄cycloalkyl containing a heteroatom, said heteroatom being an oxygen atom; and wherein R²⁷ is C₁₋₆alkyl;
   and all other variables are as defined herein.

In another embodiment, the invention relates to a compound of Formula (I) as defined herein, wherein

R⁵ is selected from the group consisting of Cycle2 and Aryl3;
wherein Cycle2 is selected from the group consisting of
C₃₋₆cycloalkyl and C₃₋₆cycloalkyl containing SO₂ or a heteroatom selected from the group consisting of oxygen and nitrogen, each of which being substituted with CONHR^{20b}, wherein R^{20b} is C₁₋₆alkyl or C₃₋₆cycloalkyl;
   wherein Aryl3 is selected from the group consisting of
phenyl or a 5- to 6-membered monocyclic heteroaryl selected from the group consisting of pyrazolyl, imidazolyl, oxazolyl, isoxazolyl, thiazolyl, triazolyl, pyridyl, pyridazinyl, pyrimidinyl, and pyrazinyl, each of which being substituted with one or more substituents each independently selected from the group consisting of C₁₋₆alkyl and CONR²⁵R²⁶; and
   wherein R²⁵ is hydrogen or CH₃;
   wherein R²⁶ is selected from the group consisting of
C₁₋₆alkyl,
C₃₋₄cycloalkyl, and
C₃₋₄cycloalkyl containing a heteroatom, said heteroatom being an oxygen atom
   and all other variables are as defined herein.

In another embodiment, the invention relates to a compound of Formula (I) as defined herein, with the proviso that when R⁴ is selected from the group consisting of N(CH₃)₂, pyrrolidin-1-yl, piperidin-1-yl, 2-methyl-piperidin-1-yl, 4-methyl-piperidin-1-yl, morpholin-1-yl, or 2,6-dimethyl-piperidin-4-yl, then R⁵ is not hydrogen.

Compounds wherein R⁴ is OH and A, R¹-R³ and R⁵-R⁶ are as defined herein are useful as synthetic intermediates and/or are isolated in the synthesis of Compounds of Formula (I). Thus, in an additional aspect, the invention relates to a compound of Formula (I-t)

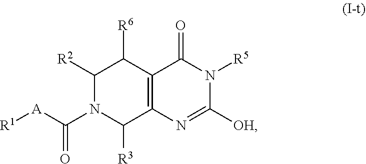

wherein
A, R¹-R³ and R⁵-R⁶ are as defined herein for compounds of Formula (I).

In another embodiment, the invention relates to a compound of Formula (I) as defined herein, wherein R⁵ is selected from the group consisting of hydrogen, C₁₋₆alkyl, C₂₋₃alkenyl, Cycle2 and Aryl3; wherein Cycle2 and Aryl3 are as defined herein.

In another embodiment, the invention relates to a compound of Formula (I) as defined herein, wherein R⁶ is hydrogen or CH₃; and all other variables are as defined herein.

The invention relates in particular, to a compound of Formula (I)

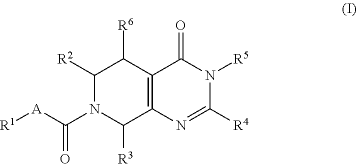

including any of its stereoisomers or tautomeric forms thereof, or a pharmaceutically acceptable salt thereof, wherein:
A is a bond or NH;
R¹ is a 5- to 10-membered monocyclic or bicyclic ring, optionally containing 1 to 3 heteroatoms, the heteroatoms being independently selected from N, O and S;
wherein the 5- to 10-membered monocyclic or bicyclic ring is optionally substituted with one or more substituents selected from hydrogen, halogens, CN, CF₃, CF₂H, CFH₂, CF₂CH₃, C₁₋₆alkyl, OC₁₋₆alkyl, OCF₃, OCF₂H and C₃₋₄cycloalkyl;
R² is selected from the group consisting of hydrogen, C₁₋₆alkyl, CF₃, CHF₂, CH₂F, phenyl and fluorophenyl;
R³ is hydrogen;

### R⁴ is X—R′;

wherein X is NR″, S or O;
wherein R′ is hydrogen, C₁₋₄alkyl, C₁₋₆alkyl substituted with OH, or C₂₋₃alkenyl, when X is NR″;
wherein R′ is C₁₋₆alkyl, when X is S;
wherein R′ is C₁₋₆alkyl, when X is O;
wherein R″ is selected from the group consisting of hydrogen, Cycle1, Aryl1, C₂₋₄alkynyl, C₁₋₆alkyl and C₁₋₆alkyl substituted with one or more substituents selected from the group consisting of
fluoro,
OH,
CO₂R¹⁶,
OCONHR¹⁷,
C₃₋₆cycloalkyl, and C₃₋₆cycloalkyl substituted with one or more from among C₁₋₆alkyl,
N-acetyl piperidine,
benzo[d][1,3]dioxole and
Aryl2;
   wherein R¹⁶ is hydrogen or C₁₋₆alkyl;
   wherein R¹⁷ is C₁₋₆alkyl;
   wherein Cycle1 is selected from the group consisting of
C₃₋₈cycloalkyl
C₃₋₈cycloalkyl containing a heteroatom, said heteroatom being an oxygen atom,
C₃₋₈cycloalkyl substituted with one or more substituents selected from CH₃ and Aryl2,
C₃₋₈cycloalkyl containing a heteroatom and being substituted with one or more substituents selected from CH₃ and Aryl2, said heteroatom being an oxygen atom,
a 5-9 membered fused bicyclic unsaturated or saturated ring,
a 5-9 membered bridged bicyclic unsaturated or saturated ring, and
a C₅₋₁₂spirocycloalkyl;
   wherein Aryl1 is selected from the group consisting of phenyl, monocyclic heteroaryl, and bicyclic heteroaryl, said Aryl1 being optionally substituted with CH₃;
   wherein Aryl2 is selected from the group consisting of phenyl, monocyclic heteroaryl, and bicyclic heteroaryl, said Aryl2 being optionally substituted with one or more substituents selected from the group consisting of halogens CF₃, CF₂H, CH₂F, C₁₋₄alkyl, C₃₋₆cycloalkyl, CN, CONR¹⁸R¹⁹, OCF₃, OCF₂H, OCH₂F, OC₁₋₄alkyl, OC₃₋₆cycloalkyl, and SO₂CH₃;
   wherein R¹⁸ and R¹⁹ are independently selected from the group consisting of hydrogen, C₁₋₆alkyl and C₃₋₆cycloalkyl;
   or wherein R′ and R″ together form a cycle selected from the group consisting of
a C₃₋₈cycloalkyl ring,
a C₃₋₈cycloalkyl ring containing a heteroatom, said heteroatom being an oxygen atom,
a C₃₋₈cycloalkyl ring substituted with one or more substituents selected from C₁₋₆alkyl, phenyl, C₂₋₆alkynyl and C₃₋₆cycloalkyl,
a C₃₋₈cycloalkyl ring containing a heteroatom and being substituted with one or more substituents selected from C₁₋₆alkyl, phenyl, C₂₋₆alkynyl and C₃₋₆cycloalkyl, said heteroatom being an oxygen atom and
a C₅₋₁₂-spirocycloalkyl;
   R⁵ is selected from the group consisting of hydrogen, C₁₋₆alkyl, C₂₋₃alkenyl, Cycle2 and Aryl3;
   wherein C₁₋₆alkyl is optionally substituted with one or more substituents selected from the group consisting of phenyl, methoxyphenyl, OC₁₋₆alkyl, NHSO₂CH₃, C₃₋₆cycloalkyl, and C₃₋₆cycloalkyl containing a heteroatom, said heteroatom being an oxygen atom;
   wherein Cycle2 is selected from the group consisting of
C₃₋₆cycloalkyl,
C₃₋₆cycloalkyl containing SO₂ or a heteroatom, the heteroatom being selected from the group consisting of oxygen and nitrogen,
C₃₋₆cycloalkyl substituted with CO₂R^{20a}, CONHC₁₋₆alkyl or SO₂C₁₋₆alkyl,
C₃₋₆cycloalkyl containing SO₂ or a heteroatom and being substituted with CO₂R^{20a}, CONHC₁₋₆alkyl or SO₂C₁₋₆alkyl, the heteroatom being selected from the group consisting of oxygen and nitrogen,
a 5-membered bridged bicyclic saturated ring substituted with CO₂C₁₋₆alkyl or CONHC₁₋₆alkyl,
isoindoline-1-one, and
indoline-2-one;
   wherein R^{20a} is hydrogen or C₁₋₆alkyl;
   wherein Aryl3 is selected from the group consisting of phenyl, monocyclic heteroaryl, and bicyclic heteroaryl, said Aryl3 being optionally substituted with one or more substituents selected from the group consisting of halogen, C₁₋₆alkyl, CF₃, CF₂H, CH₂F, CN, OC₁₋₆alkyl, OCF₃, OCF₂H, OCH₂F, OC₃₋₆cycloalkyl, SO₂R²¹, SO₂NHR²², CO₂R²³, COR²⁴, CONR²⁵R²⁶, NHR²⁷, NHCOR²⁸, Cycle3 and Aryl4;
   wherein R²¹ is C₁₋₆alkyl or C₃₋₆cycloalkyl;
   wherein R²² is C₁₋₆alkyl or pyridine;
   wherein R²³ is hydrogen or C₁₋₆alkyl;
   wherein R²⁴ is selected from the group consisting of C₁₋₆alkyl, C₅₋₆heterocycle and C₅₋₆heterocycle substituted with CH₃;
   wherein R²⁵ is hydrogen or CH₃;
   wherein R²⁶ is selected from the group consisting of
hydrogen,
C₁₋₆alkyl,
C₁₋₆alkyl optionally substituted with one or more substituents selected from the group consisting of OH, OCH₃, NH₂, CO₂H, C₃₋₆heterocycloalkyl and C₃₋₆heterocycloalkyl substituted with CH₃,
C₃₋₄cycloalkyl;
C₃₋₄cycloalkyl containing a heteroatom, said heteroatom being an oxygen atom;
C₃₋₄cycloalkyl substituted with CO₂H; and
C₃₋₄cycloalkyl containing a heteroatom and being substituted with CO₂H, said heteroatom being an oxygen atom;
   wherein R²⁷ is selected from the group consisting of
C₁₋₆alkyl,
C₁₋₆alkyl substituted with C₃₋₆heterocycloalkyl, and
C₃₋₆heterocycloalkyl;
   wherein R²⁸ is C₁₋₆alkyl or C₃₋₆cycloalkyl;
   wherein Cycle3 is selected from the group consisting of
C₃₋₆heterocycloalkyl,
C₃₋₆heterocycloalkyl substituted with one or more substituents selected from the group consisting of OH, CH₂OH, CO₂R²⁹, NHCH₃ or NHCO₂t-Bu; and imidazolidin-4-one substituted with CH₃;
   wherein R²⁹ is hydrogen or C₁₋₆alkyl;
   wherein Aryl4 is selected from the group consisting of monocyclic heteroaryl and bicyclic heteroaryl, said monocyclic or bicyclic heteroaryl being optionally substituted with one or two substituents selected from the group consisting of halogens, CF₃, CH₂F, C₁₋₆alkyl, C₃₋₆cycloalkyl, OCF₃, OCH₂F, OC₁₋₆alkyl, OC₃₋₆cycloalkyl, CO₂R³⁰, SO₂CH₃, and morpholine;
   wherein R³⁰ is hydrogen or C₁₋₆alkyl;
   wherein R′, R″ and R⁵ are not all hydrogen; and R⁵ is not CH(Ph)₂ when R⁴ is NH₂; and
   wherein R⁶ is hydrogen, CH₃, CF₃ or CF₂H.

In an embodiment, R¹ is a 5- or 6-membered (aromatic) monocyclic ring or a 9-membered bicyclic ring, optionally containing 1 to 3 heteroatoms, the heteroatoms being independently selected from N, O and S;

wherein the 5-, 6- or 9-membered ring is optionally substituted with one or more substituents selected from hydrogen, halogens, CN, CF₃, CF₂H, CFH₂, CF₂CH₃, C₁₋₆alkyl, OC₁₋₆alkyl, OCF₃, OCF₂H and C₃₋₄cycloalkyl.

In an embodiment, R¹ is a 5- to 10-membered monocyclic or bicyclic ring, more particularly a 5- to 9-membered monocyclic or bicyclic ring, optionally containing 1 to 3 heteroatoms, the heteroatoms being independently selected from N, O and S; wherein the 5- to 10-membered monocyclic or bicyclic ring, more particularly the 5- to 9-membered monocyclic or bicyclic ring, is optionally substituted with at least two, at least three, or at least four substituents selected from hydrogen, halogens, CN, CF₃, CF₂H, CFH₂, CF₂CH₃, C₁₋₆alkyl, OC₁₋₆alkyl, OCF₃, OCF₂H and C₃₋₄cycloalkyl.

In an embodiment, R¹ is a 5- to 10-membered monocyclic or bicyclic ring, more particularly a 5- to 9-membered monocyclic or bicyclic ring, optionally containing 1 to 3 heteroatoms, the heteroatoms being independently selected from N, O and S; wherein the 5- to 10-membered monocyclic or bicyclic ring, more particularly the 5- to 9-membered monocyclic or bicyclic ring, is optionally substituted with at most two, at most three, or at most four substituents selected from hydrogen, halogens, CN, CF₃, CF₂H, CFH₂, CF₂CH₃, C₁₋₆alkyl, OC₁₋₆alkyl, OCF₃, OCF₂H and C₃₋₄cycloalkyl.

In an embodiment, R¹ is a 5- to 10-membered monocyclic or bicyclic ring, more particularly a 5- to 9-membered monocyclic or bicyclic ring, optionally containing 1 to 3 heteroatoms, the heteroatoms being selected from N, O and S;

wherein the 5- to 10-membered monocyclic or bicyclic ring, more particularly a 5- to 9-membered monocyclic or bicyclic ring, is optionally substituted with one, two, three, or four substituents selected from hydrogen, halogens, CN, CF₃, CF₂H, CFH₂, CF₂CH₃, C₁₋₆alkyl, OC₁₋₆alkyl, OCF₃, OCF₂H and C₃₋₄cycloalkyl.

In an embodiment, R¹ is a phenyl substituted with one or more substituents selected from hydrogen, halogens, CN, CF₃, CF₂H, CFH₂, CF₂CH₃, C₁₋₆alkyl, OC₁₋₆alkyl, OCF₃, OCF₂H and C₃₋₄cycloalkyl.

In an embodiment, R¹ is a 6-membered heteroaryl containing a nitrogen atom and substituted with one or more substituents selected from hydrogen, halogens, CN, CF₃, CF₂H, CFH₂, CF₂CH₃, C₁₋₆alkyl, OC₁₋₆alkyl, OCF₃, OCF₂H and C₃₋₄cycloalkyl.

In an embodiment, R¹ is a ring of formula (II) or of formula (III):

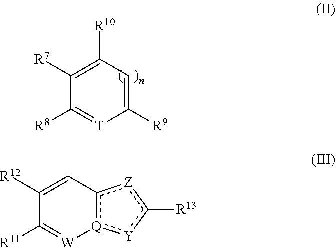

wherein:
n is an integer of 0 or 1;
T is S when n is 0;
T is CR¹⁴ or a nitrogen when n is 1;
R⁷ is hydrogen, halogen, CN, CF₃, CHF₂, C₁₋₆alkyl or OC₁₋₆alkyl;
R⁸ is hydrogen, halogen, CF₃, CHF₂, CN, cyclopropyl, C₁₋₆alkyl or OC₁₋₆alkyl;
R⁹ is C₁₋₄alkyl or the attachment point of the radical;
R¹⁰ is hydrogen or the attachment point of the radical;
R¹⁴ is hydrogen or fluoro, provided that R⁷, R⁸ and R¹⁴ are not all halogen;

### W is CH or N;

### Q is C or N;

### Y is CH, N, NH, O or S;

Z is N, O, CH₂ or CR¹⁵; [symbol]
indicates a saturated or unsaturated bond, whereby Q, Y, and Z are selected such that at least one bond [symbol] is unsaturated;
R¹¹ is hydrogen, fluoro or chloro;
R¹² is hydrogen, fluoro or the attachment of the radical;
R¹³ is hydrogen or the attachment point of the radical; and
R¹⁵ is hydrogen, chloro or methyl.

In an embodiment, R¹ is a ring of Formula (II), and R⁹ is the attachment point of the radical, and R¹⁰ is hydrogen.

In an embodiment, R¹ is a ring of Formula (IV):

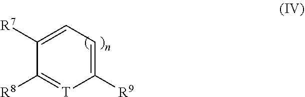

wherein:
n is an integer of 0 or 1;
T is S when n is 0;
T is CR¹⁴ or a nitrogen when n is 1;
R⁷ is halogen, CN, CF₃, CHF₂, C₁₋₆alkyl or OC₁₋₆alkyl;
R⁸ is hydrogen, halogen, CF₃, CHF₂, CN, cyclopropyl, C₁₋₆alkyl or OC₁₋₆alkyl;
R⁹ is the attachment point of the radical; and
R¹⁴ is hydrogen or fluoro, provided that R¹, R⁸ and R¹⁴ are not all halogen.

In an embodiment, n is 1, and T is CR¹⁴ or a nitrogen.

In an embodiment, R¹ is a ring of Formula (V):

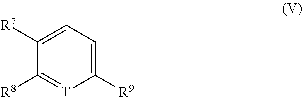

wherein:
T is CR¹⁴ or a nitrogen;
R⁷ is halogen, CN, CF₃, CHF₂, C₁₋₆alkyl or OC₁₋₆alkyl;
R⁸ is hydrogen, halogen, CF₃, CHF₂, CN, cyclopropyl, C₁₋₆alkyl or OC₁₋₆alkyl;
R⁹ is the attachment point of the radical; and
R¹⁴ is hydrogen or fluoro, provided that R⁷, R⁸ and R¹⁴ are not all halogen;

In an embodiment, T is CR¹⁴.

In an embodiment, R¹ is a ring of Formula (VI):

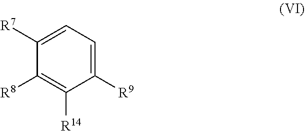

wherein:
R⁷ is halogen, CN, CF₃, CHF₂, C₁₋₆alkyl or OC₁₋₆alkyl;
R⁸ is hydrogen, halogen, CF₃, CHF₂, CN, cyclopropyl, C₁₋₆alkyl or OC₁₋₆alkyl;
R⁹ is the attachment point of the radical; and
R¹⁴ is hydrogen or fluoro, provided that R⁷, R⁸ and R¹⁴ are not all halogen.

In a yet further embodiment, the invention relates to a compound of Formula (I) as defined herein, wherein R¹ is

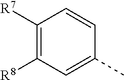

wherein:
R⁷ is halogen, in particular chloro; and
R⁸ is selected from the group consisting of halogen, CF₃, CHF₂, and C₁₋₆alkyl; and all other variables are as defined herein.

The application relates more particularly to those compounds as defined herein which show an EC₅₀ of less than 0.50 μM for the inhibition of HBV DNA for example in the HepG2.117 cell line, more particularly an EC₅₀ of less than 0.50 μM for the inhibition of HBV DNA when measured 3 days after the compound has been placed in the HepG2.117 cell culture. Typically, for measuring the EC₅₀ value of a compound as defined herein, HepG2.117 cells can be cultured in the presence of DMSO or of the test compound in absence of doxycycline (HepG2 cell line available from ATCCO under number HB-8065; transfection of the HepG2 cell line as described in Sun and Nassal, Journal of Hepatology 45 (2006) 636-645 “Stable HepG2- and Huh7-based human hepatoma cell lines for efficient regulated expression of infectious hepatitis B virus”).

In an embodiment, R¹ is a ring of Formula (III), W is CH, and Q is C.

In an embodiment, R¹ is a ring of Formula (VII):

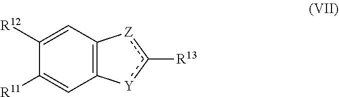

wherein:

### Y is CH, N, NH, O or S;

Z is N, O, CH₂ or CR¹⁵; [symbol]
indicates a saturated or unsaturated bond, whereby Y and Z are selected such that at least one bond [symbol] is unsaturated or the two bonds are saturated;
R¹¹ is hydrogen, fluoro or chloro;
R¹² is hydrogen, fluoro or the attachment of the radical;
R¹³ is hydrogen or the attachment point of the radical; and
R¹⁵ is hydrogen, chloro or methyl.

In an embodiment, Y is NH, O or S, and Z is N, CH₂ or CR¹⁵.

In an embodiment, R¹ is a ring of Formula (VIII):

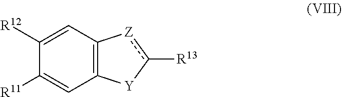

wherein:

### Y is NH, O or S;

Z is N, CH₂ or CR¹⁵; [symbol]
indicates a saturated or unsaturated bond;
R¹¹ is hydrogen, fluoro or chloro;
R¹² is hydrogen, fluoro or the attachment of the radical;
R¹³ is hydrogen or the attachment point of the radical; and
R¹⁵ is hydrogen, chloro or methyl;

In an embodiment, Z is N or CR¹⁵.

In an embodiment, R¹ is a ring of Formula (IX):

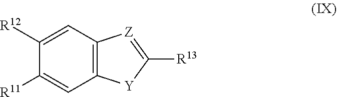

wherein:

### Y is NH, O or S;

### Z is N, or CR¹⁵;

R¹¹ is hydrogen, fluoro or chloro;
R¹² is hydrogen, fluoro or the attachment of the radical;
R¹³ is hydrogen or the attachment point of the radical; and
R¹⁵ is hydrogen, chloro or methyl.

In an embodiment, Z is CR¹⁵, R¹² is hydrogen or fluoro, and R¹³ is the attachment point of the radical.

In an embodiment R¹ is a ring of formula (X):

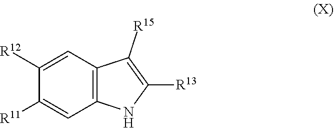

wherein:
R¹¹ is hydrogen, fluoro or chloro;
R¹² is hydrogen, or fluoro;
R¹³ is the attachment point of the radical; and
R¹⁵ is hydrogen, chloro or methyl.

In an embodiment, R′ is hydrogen.

In an embodiment, R⁴ is NR′R″;

wherein R′ is hydrogen;
wherein R″ is selected from the group consisting of Cycle1, C₁₋₆alkyl and C₁₋₆alkyl substituted with Aryl2;
wherein Cycle1 is C₃₋₈cycloalkyl substituted with one or more substituents each independently selected from CH₃ and Aryl2; or C₃₋₈cycloalkyl containing a heteroatom and being substituted with one or more substituents each independently selected from CH₃ and Aryl2, said heteroatom being an oxygen atom; and
wherein Aryl2 is phenyl optionally substituted with one or more substituents each independently selected from the group consisting of halogens CF₃, CF₂H, CH₂F, C₁₋₄alkyl, C₃₋₆cycloalkyl, CN, CONR¹⁸R¹⁹, OCF₃, OCF₂H, OCH₂F, OC₁₋₄alkyl, OC₃₋₆cycloalkyl, and SO₂CH₃;
wherein R¹⁸ and R¹⁹ are independently selected from the group consisting of hydrogen, C₁₋₆alkyl and C₃₋₆cycloalkyl;
or wherein R′ and R″ together form a cycle selected from the group consisting of
a C₃₋₈cycloalkyl ring,
a C₃₋₈cycloalkyl ring containing a heteroatom, said heteroatom being an oxygen atom, and
a C₃₋₈cycloalkyl ring substituted with one or more substituents each independently selected from the group consisting of C₁₋₆alkyl, phenyl, C₂₋₆alkynyl and C₃₋₆cycloalkyl.

In an embodiment, R⁷ and R⁸ are each independently halogen, particularly chloro.

In an embodiment, X is NR″ and R′ is H.

In an embodiment, R″ is C₁₋₆alkyl substituted with one or more substituents selected from the group consisting of fluoro, OH, CO₂R¹⁶, OCONHR¹⁷, C₃₋₆cycloalkyl, and C₃₋₆cycloalkyl substituted with one or more from among C₁₋₆alkyl, N-acetyl piperidine, benzo[d][1,3]dioxole and Aryl2.

In an embodiment, Aryl2 is phenyl or phenyl substituted with one or more substituents selected from the group consisting of halogens, CF₃, CF₂H, CH₂F, C₁₋₄alkyl, C₃₋₆cycloalkyl, CN, CONR¹⁸R¹⁹, OCF₃, OCF₂H, OCH₂F, OC₁₋₄alkyl, OC₃₋₆cycloalkyl and SO₂CH₃.

In an embodiment, R″ is C₁₋₆alkyl.

In an embodiment, R⁵ is Cycle2 or Aryl3;

wherein Cycle2 is selected from the group consisting of
C₃₋₆cycloalkyl or C₃₋₆cycloalkyl containing SO₂ or a heteroatom and being substituted with CONHC₁₋₆alkyl, the heteroatom being selected from the group consisting of oxygen and nitrogen;
   wherein Aryl3 is selected from the group consisting of phenyl and monocyclic heteroaryl, said Aryl3 being substituted with CONR²⁵R²⁶
   wherein R²⁵ is hydrogen or CH₃;
   wherein R²⁶ is selected from the group consisting of
C₁₋₆alkyl,
C₁₋₆alkyl optionally substituted with one or more substituents each independently selected from the group consisting of OH, OCH₃, NH₂, CO₂H, C₃₋₆heterocycloalkyl and C₃₋₆heterocycloalkyl substituted with CH₃,
C₃₋₄cycloalkyl; and
C₃₋₄cycloalkyl containing a heteroatom, said heteroatom being an oxygen atom.

In an embodiment, R⁵ is Aryl3.

In an embodiment, Aryl3 is phenyl or phenyl substituted with one or more substituents selected from the group consisting of halogen, C₁₋₆alkyl, CF₃, CF₂H, CH₂F, CN, OC₁₋₆alkyl, OCF₃, OCF₂H, OCH₂F, OC₃₋₆cycloalkyl, SO₂R²¹, SO₂NHR²², CO₂R²³, COR²⁴, CONR²⁵R²⁶, NHR²⁷, NHCOR²⁸, Cycle3 and Aryl4.

In an embodiment, A is a bond.

In an embodiment, R³ and R⁶ are both hydrogen.

Examples of compounds as defined herein are listed in Section 3 below.

### Medical Application

The compounds of the application can be useful for simultaneous, separate or sequential use in the treatment of chronic Hepatitis B or of HBV-induced diseases.

HBV-induced diseases can be selected from the group consisting of liver fibrosis, liver inflammation, liver necrosis, cirrhosis, end-stage liver disease, and hepatocellular carcinoma.

In one aspect, the application pertains to a method of treatment or prevention of a subject in need thereof, comprising administering to a subject in need thereof with a therapeutically effective amount of the compound or pharmaceutically acceptable salt thereof as defined herein or the pharmaceutical composition as defined herein.

In yet another aspect, the application pertains to a method of treating or preventing an HBV infection or of an HBV-induced disease in a mammal, said method comprising administering to said mammal a therapeutically effective amount of a compound or pharmaceutically acceptable salt thereof as defined herein, or the pharmaceutical composition as defined herein.

In yet another aspect, the application pertains to a method of treating or preventing chronic Hepatitis B in a subject in need thereof, said method comprising administering to said subject a therapeutically effective amount of a compound or pharmaceutically acceptable salt thereof as defined herein, or the pharmaceutical composition as defined herein.

In yet another aspect, the application pertains to a method of treating or preventing HBV-induced disease Hepatitis B in a subject, said method comprising administering to said subject a compound or pharmaceutically acceptable salt as defined herein, or the pharmaceutical composition as defined herein, wherein the HBV-induced disease is selected from the group consisting of liver fibrosis, liver inflammation, liver necrosis, cirrhosis, end-stage liver disease, and hepatocellular carcinoma.

The application relates to such a compound or pharmaceutically acceptable salt thereof, or to such a pharmaceutical composition, for use in the prevention, the prevention of aggravation, the amelioration or the treatment of a HBV-induced disease or condition.

The application relates to such a compound or pharmaceutically acceptable salt, or to such a pharmaceutical composition, for any of the above-mentioned uses, more particularly for use in the prevention, the prevention of aggravation, the amelioration, or the treatment of one or more of the following items:
the prevention of chronic hepatitis infection, more particularly chronic hepatitis B infection (ie, preventing that the hepatitis (B) infection becomes chronic);
the amelioration or treatment of a hepatitis-associated or hepatitis-induced (chronic) disease or condition, more particularly of a hepatitis B-associated or hepatitis B-induced (chronic) disease or condition;
the prevention of the aggravation of a hepatitis-associated or hepatitis-induced (chronic) disease or condition, more particularly of a hepatitis B-associated or hepatitis B-induced (chronic) disease or condition;
the amelioration (regression, or absence of progression) of the stage of liver fibrosis, or of the extent of liver damage, induced by a (chronic) hepatitis infection, more particularly by a (chronic) hepatitis B infection;
the amelioration (reduction) of the fibrosis progression rate of a (chronic) hepatitis infection, more particularly the prevention of cirrhosis in a subject having a (chronic) hepatitis infection, more particularly by a (chronic) hepatitis B infection (e.g., preventing that the subject reaches the cirrhotic stage of fibrosis).

The compounds of the invention may also exist in unsolvated and solvated forms. The term “solvate” is used herein to describe a molecular complex comprising the compound of the invention and one or more pharmaceutically acceptable solvent molecules, for example, ethanol.

The term “polymorph” refers to the ability of the compound of the invention to exist in more than one form or crystal structure.

In yet another aspect, the application pertains to a method of treating or preventing an HBV infection or of an HBV-induced disease in a mammal, said method comprising administering to said mammal a product comprising a first compound and a second compound as a combined preparation for simultaneous, separate or sequential use, wherein said first compound is different from said second compound, wherein said first compound is the compound or pharmaceutically acceptable salt as defined herein or the pharmaceutical composition as defined herein, and wherein said second compound is another HBV inhibitor which is selected from the group consisting of: therapeutic agents selected from HBV combination drugs, HBV vaccines, HBV DNA polymerase inhibitors, immunomodulators, toll-like receptor (TLR) modulators, interferon alpha receptor ligands, hyaluronidase inhibitors, hepatitis b surface antigen (HBsAg) inhibitors, cytotoxic T-lymphocyte-associated protein 4 (ipi4) inhibitors, cyclophilin inhibitors, HBV viral entry inhibitors, antisense oligonucleotide targeting viral mRNA, short interfering RNAs (siRNA) and ddRNAi endonuclease modulators, ribonucleotide reductase inhibitors, HBV E antigen inhibitors, covalently closed circular DNA (cccDNA) inhibitors, farnsoid X receptor agonists, HBV antibodies, CCR2 chemokine antagonists, thymosin agonists, cytokines, nucleoprotein modulators, retinoic acid-inducible gene 1 simulators, NOD2 stimulators, phosphatidylinositol 3-kinase (PI3K) inhibitors, indoleamine 2, 3-dioxygenase (IDO) pathway inhibitors, PD-1 inhibitors, PD-L1 inhibitors, recombinant thymosin alpha-1, bruton's tyrosine kinase (BTK) inhibitors, KDM inhibitors, HBV replication inhibitors, arginase inhibitors, and other anti-HBV drugs.

### Administration/Dosage/Formulations

In another aspect, provided herein is a pharmaceutical composition comprising at least one disclosed compound, or a pharmaceutically acceptable salt thereof, together with a pharmaceutically acceptable carrier.

The compounds of the present invention may be administered as crystalline or amorphous products. They may be obtained for example as solid plugs, powders, or films by methods such as precipitation, crystallization, freeze drying, spray drying, or evaporative drying. They may be administered alone or in combination with one or more other compounds of the invention or in combination with one or more other drugs. Generally, they will be administered as a formulation in association with one or more pharmaceutically acceptable excipients. The term “excipient” is used herein to describe any ingredient other than the compound(s) of the invention. The choice of excipient depends largely on factors such as the particular mode of administration, the effect of the excipient on solubility and stability, and the nature of the dosage form.

The compounds of the present invention or any subgroup thereof may be formulated into various pharmaceutical forms for administration purposes. As appropriate compositions there may be cited all compositions usually employed for systemically administering drugs. To prepare the pharmaceutical compositions of this invention, an effective amount of the particular compound, optionally in addition salt form, as the active ingredient is combined in intimate admixture with a pharmaceutically acceptable carrier, which carrier may take a wide variety of forms depending on the form of preparation desired for administration. These pharmaceutical compositions are desirably in unitary dosage form suitable, for example, for oral, rectal, or percutaneous administration. For example, in preparing the compositions in oral dosage form, any of the usual pharmaceutical media may be employed such as, for example, water, glycols, oils, alcohols and the like in the case of oral liquid preparations such as suspensions, syrups, elixirs, emulsions, and solutions; or solid carriers such as starches, sugars, kaolin, diluents, lubricants, binders, disintegrating agents and the like in the case of powders, pills, capsules, and tablets. Because of their ease in administration, tablets and capsules represent the most advantageous oral dosage unit forms, in which case solid pharmaceutical carriers are obviously employed. Also included are solid form preparations that can be converted, shortly before use, to liquid forms. In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment. The compounds of the present invention may also be administered via inhalation or insufflation by means of methods and formulations employed in the art for administration via this way. Thus, in general the compounds of the present invention may be administered to the lungs in the form of a solution, a suspension or a dry powder.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level will depend upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could begin administration of the pharmaceutical composition to dose the disclosed compound at levels lower than that required in order to achieve the desired therapeutic effect and gradually increase the dosage until the desired effect is achieved.

Furthermore, it is evident that the effective amount may be lowered or increased depending on the response of the treated subject and/or depending on the evaluation of the physician prescribing the compounds of the instant invention. The effective amount ranges mentioned herein are therefore only guidelines and are not intended to limit the scope or use of the invention to any extent.

Those of skill in the treatment of infectious diseases will be able to determine the effective amount from the test results presented hereinafter. In general it is contemplated that an effective daily amount would be from 0.01 mg/kg to 50 mg/kg body weight, more preferably from 0.1 mg/kg to 10 mg/kg body weight. It may be appropriate to administer the required dose as two, three, four or more sub-doses at appropriate intervals throughout the day.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of the disclosed compound calculated to produce the desired therapeutic effect in association with the required pharmaceutical carrier. Examples of such unit dosage forms are tablets (including scored or coated tablets), capsules, pills, powder packets, wafers, suppositories, injectable solutions or suspensions and the like, and segregated multiples thereof.

The dosage unit forms of the invention are dictated by and directly dependent on (a) the unique characteristics of the disclosed compound and the particular therapeutic effect to be achieved, and (b) the limitations inherent in the art of compounding/formulating such a disclosed compound for the treatment of HBV infection in a patient.

In an embodiment, the compositions of the invention are formulated using one or more pharmaceutically acceptable excipients or carriers. In an embodiment, the pharmaceutical compositions of the invention comprise a therapeutically effective amount of a disclosed compound and a pharmaceutically acceptable carrier.

In some embodiments, the dose of a disclosed compound is from about 1 mg to about 2,500 mg. In some embodiments, a dose of a disclosed compound used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., another drug for HBV treatment) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In an embodiment, the present invention is directed to a packaged pharmaceutical composition comprising a container holding a therapeutically effective amount of a disclosed compound, alone or in combination with a second pharmaceutical agent, and instructions for using the compound to treat, prevent, or reduce one or more symptoms of HBV infection in a patient.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent. For parenteral administration, the disclosed compounds may be formulated for injection or infusion, for example, intravenous, intramuscular or subcutaneous injection or infusion, or for administration in a bolus dose or continuous infusion. Suspensions, solutions or emulsions in an oily or aqueous vehicle, optionally containing other formulatory agents such as suspending, stabilizing or dispersing agents may be used.

As used herein, the terms and phrases “simultaneous use”, “separate use” or “sequential use” in the context of the administration of two or more therapies or components to a subject refers to administration of two or more therapies or components, for example a compound according to Formula (I) and at least one other compound.

As used herein, “simultaneous use” can be administration of the two or more components at essentially the same moment in time. In particular, during simultaneous use, the two or more components can be administered in one composition or in multiple, in particular separate, compositions. More particularly, during simulateneous use, when the two or more components are administered in multiple compositions, said compositions may be administered via the same or via a different route.

As used herein, “separate use” can indicate that the two or more components are provided separately, in particular in two or more compositions.

As used herein, when the two or more components are for “sequential use”, they can be administered in multiple, in particular separate, compositions sequentially within a short time period, such as within 24, 20, 16, 12, 8 or 4 hours, within 1 hour, within 45, 30, 20, 15, 10, 5, 4, 3, or 2 minutes, or within 1 minute.

The use of the term “in combination with” does not restrict the order in which therapies or components are administered to a subject. For example, a first therapy or component (e.g. a first compound according to Formula (I)) can be administered prior to (e.g., 5 minutes to one hour before), concomitantly with or simultaneously with, or subsequent to (e.g., 5 minutes to one hour after) the administration of a second therapy or component (e.g., a second compound according to Formula (I)). In some embodiments, a first therapy or component (e.g. a first compound according to Formula (I)) and a second therapy or component (e.g, a second compound according to Formula (I)) are administered in the same composition. In other embodiments, a first therapy or component (e.g. a first compound according to Formula (I)) and a second therapy or component (e.g., a second compound according to Formula (I)) are administered in separate compositions.

### Methods

The application relates to a method for the preparation of a compound as described herein. The method comprises the step of providing a compound according to Formula (XI):

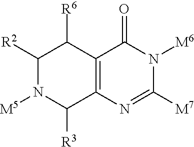

Formula (XI) or a tautomer thereof;
wherein
   M⁵ is selected from the group consisting of tert-butyloxycarbonyl, hydrogen, and R¹-A-C(O);

### M⁶ is H; and

M⁷ is selected from the group consisting of sulfhydryl, —SCH₃, —Cl, —S(O)CH₃, and R⁴;
wherein when M⁵ is R¹-A-C(O)— and M⁶ is H, then M⁷ is not R⁴;
wherein when M⁵ and M⁶ are hydrogen, then M⁷ is not —NH₂;
   wherein the method further comprises at least one of steps a) to h):
a) contacting the compound according to Formula (XI) with a strong acid, more particularly hydrochloric acid or trifluoroacetic acid;
b) contacting the compound according to Formula (XI) with R¹-A-C(O)-G, more particularly in the presence of a non-nucleophilic base;
c) contacting the compound according to Formula (XI) with M¹⁰-H^{A}, more particularly in the presence of Cs₂CO₃, wherein M¹⁰ is selected from the group consisting of C₁₋₆alkyl, C₁₋₃alkenyl, and Cycle2; wherein C₁₋₆alkyl is optionally substituted with phenyl, methoxyphenyl, OC₁₋₆alkyl, NHSO₂CH₃, and C₃₋₆cycloalkyl optionally containing an oxygen atom;
   and wherein H^{A} is a halogen, more particularly Br;
d) contacting the compound according to Formula (XI) with Aryl3-B(OH)₂, more particularly in the presence of copper(II) acetate;
e) contacting the compound according to Formula (XI) with a methylating agent, more particularly methyl iodide;
f) contacting the compound according to Formula (XI) with an oxidizing agent, more particularly meta-chloroperoxybenzoic acid;
g) contacting the compound according to Formula (XI) with a chlorinating agent, more particularly thiophosgene;
h) contacting the compound according to Formula (XI) with one of the reagents consisting of R′—S—H, R′—O—H and R′R″N—H, more particularly in the absence or in the presence of a non-nucleophilic base;
   wherein when M⁵ is tert-butyloxycarbonyl, the method comprises at least steps a) and b);
   wherein when M⁵ is hydrogen, the method comprises at least step b);
   wherein when M⁷ is sulfhydryl the method comprises at least steps e), f) and h); or at least steps g) and h);
   wherein when M⁷ is —SCH₃, the method comprises at least steps f) and h);
   wherein when M⁷ is —Cl or —S(O)CH₃, the method comprises at least step h);
   wherein when the method comprises steps e) and/or f), the method does not comprise step g);
   wherein when the method comprises step c), the method does not comprise step d);
   and wherein:
      R¹, R², R³, R⁴, R⁵, R⁶, A, X, R′, R″, Cycle2, and Aryl3 are as defined herein; G is selected from the group consisting of OH and Cl;
when G is OH, step b) comprises contacting the compound according to Formula (XI) with R¹-A-C(O)-G in the presence of a coupling reagent; and
wherein the coupling reagent more particularly is 2-(1H-benzotriazol-1-yl)-1,1,3,3-tetramethyluronium hexafluorophosphate (HBTU).

In an embodiment, the application relates to a method for the preparation of a compound as described herein and wherein R⁵ is other than hydrogen. The method comprises the step of providing a compound according to Formula (XI):

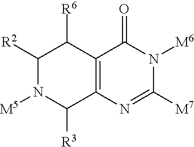

Formula (XI) or a tautomer thereof;
wherein
   M⁵ is R¹-A-C(O)—;
   M⁶ is hydrogen; and
   M⁷ is R⁴;
   wherein the method further comprises at most one of steps a) and b):
a) contacting the compound according to Formula (XI) with M¹⁰-H^{A}, more particularly in the presence of Cs₂CO₃, wherein M¹⁰ is selected from the group consisting of C₁₋₆alkyl, C₁₋₃alkenyl, and Cycle2; wherein C₁₋₆alkyl is optionally substituted with phenyl, methoxyphenyl, OC₁₋₆alkyl, NHSO₂CH₃, and C₃₋₆ cycloalkyl optionally containing an oxygen atom;
   and wherein H^{A} is a halogen, more particularly Br;
b) contacting the compound according to Formula (XI) with Aryl3-B(OH)₂, more particularly in the presence of copper(II) acetate;
   and wherein:
      R¹, R², R³, R⁴, R⁵, R⁶, A, Cycle2, and Aryl3 are as defined herein, with the proviso that R⁵ is not hydrogen.

In another embodiment, the application relates to a method for the preparation of a compound as described herein, wherein R⁵ is other than hydrogen. The method comprises the step of providing a compound according to Formula (XI):

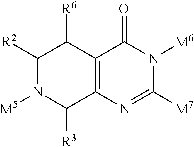

Formula (XI) or a tautomer thereof; wherein
   M⁵ is selected from the group consisting of tert-butyloxycarbonyl, hydrogen, and R¹-A-C(O);
   M⁶ is R⁵ is other than hydrogen; and
   M⁷ is selected from the group consisting of sulfhydryl, —SCH₃, —Cl, —S(O)CH₃, and R⁴;
   wherein when M⁵ is R¹-A-C(O)—, then M⁷ is not R⁴;
   wherein the method further comprises at least one of steps a) to f):
a) contacting the compound according to Formula (XI) with a strong acid, more particularly hydrochloric acid or trifluoroacetic acid;
b) contacting the compound according to Formula (XI) with R¹-A-C(O)-G, more particularly in the presence of a non-nucleophilic base;
c) contacting the compound according to Formula (XI) with a methylating agent, more particularly methyl iodide;
d) contacting the compound according to Formula (XI) with an oxidizing agent, more particularly meta-chloroperoxybenzoic acid;
e) contacting the compound according to Formula (XI) with a chlorinating agent, more particularly thiophosgene;
f) contacting the compound according to Formula (XI) with one of the reagents consisting of R′—S—H, R′—O—H and R′R″N—H, more particularly in the absence or in the presence of a non-nucleophilic base;
   wherein when M⁵ is tert-butyloxycarbonyl, the method comprises at least steps a) and b);
   wherein when M⁵ is hydrogen, the method comprises at least step b);
   wherein when M⁷ is sulfhydryl the method comprises at least steps c), d) and f); or at least steps e) and f);
   wherein when M⁷ is —SCH₃, the method comprises at least steps d) and f);
   wherein when M⁷ is —Cl or —S(O)CH₃, the method comprises at least step f);
   wherein when the method comprises steps c) and/or d), the method does not comprise step e); and wherein:
   R¹, R², R³, R⁴, R⁵, R⁶, A, X, R′, R″, Cycle2, and Aryl3 are as defined herein and with the proviso that R⁵ is other than hydrogen;
   G is selected from the group consisting of OH and Cl;
when G is OH, step b) comprises contacting the compound according to Formula (XI) with R¹-A-C(O)-G in the presence of a coupling reagent; and
wherein the coupling reagent more particularly is 2-(1H-benzotriazol-1-yl)-1,1,3,3-tetramethyluronium hexafluorophosphate (HBTU).

In yet another embodiment, the application relates to a process for the preparation of a compound as described herein. The process comprises the steps of:
a) contacting a compound of Formula (XII) with R⁵—NH₂, wherein Formula (XII) is

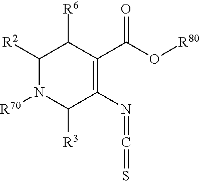

Formula (XII), to provide the compound of Formula (XIII):

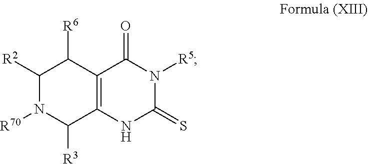

b) contacting the compound of Formula (XIII) with a methylating agent, more particularly methyl iodide, to form a compound according to Formula (XIV):

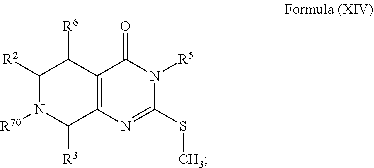

c) contacting the compound of Formula (XIV) with an oxidizing agent, more particularly meta-chloroperoxybenzoic acid, to form a compound of Formula (XV):

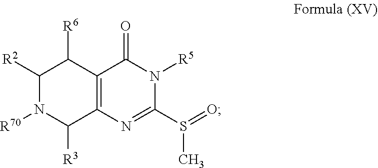

d) contacting the compound of Formula (XV) with R′R″N—H, more particularly in the presence of a non-nucleophilic base, to form a compound of Formula (XVI):

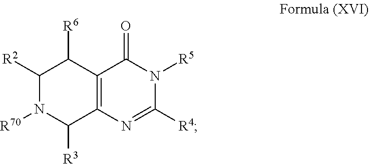

e) contacting the compound of Formula (XVI) with a strong acid, more particularly hydrochloric acid or trifluoroacetic acid, to form a compound of Formula (XVII):

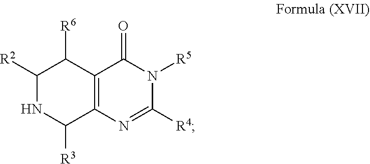

f) contacting the compound of Formula (XVII) with R¹-A-C(O)-G;
wherein:
R¹, R², R³, R⁴, R⁵, R⁶, A, X, and R′ are as defined herein;
R⁷⁰ is tert-butyloxycarbonyl;
R⁸⁰ is C₁₋₄alkyl; and
G is selected from the group consisting of OH and Cl,
when G is OH, step f) comprises contacting the compound according to Formula (XVII) with R¹-A-C(O)-G in the presence of a coupling reagent, more particularly in the presence of a non-nucleophilic base; and wherein the coupling reagent more particularly is 2-(1H-benzotriazol-1-yl)-1,1,3,3-tetramethyluronium hexafluorophosphate (HBTU).

In an embodiment, the application relates to a method for the preparation of a compound as described herein. The method comprises the step of providing a compound according to Formula (XI):

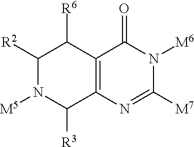

Formula (XI) or a tautomer thereof; wherein
   M⁵ is selected from the group consisting of tert-butyloxycarbonyl, hydrogen, and R¹-A-C(O);
   M⁶ is selected from the group consisting of tert-butyloxycarbonyl, and H; and
   M⁷ is selected from the group consisting of sulfhydryl, —SCH₃, —Cl, —S(O)CH₃, and R⁴;
   wherein when M⁵ is R¹-A-C(O)— and M⁶ is H, then M⁷ is not R⁴;
   wherein when M⁵ and M⁶ are hydrogen, then M⁷ is not —NH₂;
   wherein the method further comprises at least one of steps a) to h):
a) contacting the compound according to Formula (XI) with a strong acid, more particularly hydrochloric acid or trifluoroacetic acid;
b) contacting the compound according to Formula (XI) with R¹-A-C(O)-G, more particularly in the presence of a non-nucleophilic base;
c) contacting the compound according to Formula (XI) with M¹⁰-H^{A}, more particularly in the presence of Cs₂CO₃, wherein M¹⁰ is selected from the group consisting of C₁₋₆alkyl, C₁₋₃alkenyl, and Cycle2; wherein C₁₋₆alkyl is optionally substituted with phenyl, methoxyphenyl, OC₁₋₆alkyl, NHSO₂CH₃, and C₃₋₆cycloalkyl optionally containing an oxygen atom;
   and wherein H^{A} is a halogen, more particularly Br;
d) contacting the compound according to Formula (XI) with Aryl3-B(OH)₂, more particularly in the presence of copper(II) acetate;
e) contacting the compound according to Formula (XI) with a methylating agent, more particularly methyl iodide;
f) contacting the compound according to Formula (XI) with an oxidizing agent, more particularly meta-chloroperoxybenzoic acid;
g) contacting the compound according to Formula (XI) with a chlorinating agent, more particularly thiophosgene;
h) contacting the compound according to Formula (XI) with H—X—R′, more particularly in the absence or in the presence of a non-nucleophilic base;
   wherein when M⁵ is tert-butyloxycarbonyl, the method comprises at least steps a) and b);
   wherein when M⁵ is hydrogen, the method comprises at least step b);
   wherein when M⁶ is hydrogen, the method optionally comprises at least step c) or at least step d);
      wherein when M⁶ is tert-butyloxycarbonyl, the method comprises at least steps a) and c) or at least steps a) and d);
wherein when M⁷ is sulfhydryl the method comprises at least steps e), f) and h); or at least steps g) and h);
wherein when M⁷ is —SCH₃, the method comprises at least steps f) and h);
wherein when M⁷ is —Cl or —S(O)CH₃, the method comprises at least step h);
wherein when the method comprises steps e) and/or f), the method does not comprise step g);
wherein when the method comprises step c), the method does not comprise step d);
and wherein:
   R¹, R², R³, R⁴, R⁵, R⁶, A, X, R′, Cycle2, and Aryl3 are as defined herein;
   G is selected from the group consisting of OH and Cl;
when G is OH, step b) comprises contacting the compound according to Formula (XI) with R¹-A-C(O)-G in the presence of a coupling reagent; and
wherein the coupling reagent more particularly is 2-(1H-benzotriazol-1-yl)-1,1,3,3-tetramethyluronium hexafluorophosphate (HBTU).

In an embodiment, the application relates to a method for the preparation of a compound as described herein. The method comprises the step of providing a compound according to Formula (XI):

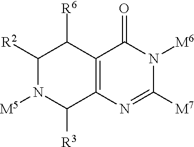

Formula (XI) or a tautomer thereof;
wherein
   M⁵ is selected from the group consisting of tert-butyloxycarbonyl, hydrogen, and R¹-A-C(O);

### M⁶ is H; and

M⁷ is selected from the group consisting of sulfhydryl, —SCH₃, —Cl, —S(O)CH₃, and R⁴;
wherein when M⁵ is R¹-A-C(O)— and M⁶ is H, then M⁷ is not R⁴;
wherein when M⁵ and M⁶ are hydrogen, then M⁷ is not —NH₂;
wherein the method further comprises at least one of steps a) to h):
a) contacting the compound according to Formula (XI) with a strong acid, more particularly hydrochloric acid or trifluoroacetic acid;
b) contacting the compound according to Formula (XI) with R¹-A-C(O)-G, more particularly in the presence of a non-nucleophilic base;
c) contacting the compound according to Formula (XI) with M¹⁰-H^{A}, more particularly in the presence of Cs₂CO₃, wherein M¹⁰ is selected from the group consisting of C₁₋₆alkyl, C₁₋₃alkenyl, and Cycle2; wherein C₁₋₆alkyl is optionally substituted with phenyl, methoxyphenyl, OC₁₋₆alkyl, NHSO₂CH₃, and C₃₋₆cycloalkyl optionally containing an oxygen atom;
and wherein H^{A} is a halogen, more particularly Br;
d) contacting the compound according to Formula (XI) with Aryl3-B(OH)₂, more particularly in the presence of copper(II) acetate;
e) contacting the compound according to Formula (XI) with a methylating agent, more particularly methyl iodide;
f) contacting the compound according to Formula (XI) with an oxidizing agent, more particularly meta-chloroperoxybenzoic acid;
g) contacting the compound according to Formula (XI) with a chlorinating agent, more particularly thiophosgene;
h) contacting the compound according to Formula (XI) with one of the reagents consisting of R′—S—H, R′—O—H and R″NH₂, more particularly in the absence or in the presence of a non-nucleophilic base;
wherein when M⁵ is tert-butyloxycarbonyl, the method comprises at least steps a) and b);
wherein when M⁵ is hydrogen, the method comprises at least step b); wherein when M⁷ is sulfhydryl the method comprises at least steps e), f) and h); or at least steps g) and h);
wherein when M⁷ is —SCH₃, the method comprises at least steps f) and h);
wherein when M⁷ is —Cl or —S(O)CH₃, the method comprises at least step h);
wherein when the method comprises steps e) and/or f), the method does not comprise step g);
wherein when the method comprises step c), the method does not comprise step d);
and wherein:
   R¹, R², R³, R⁴, R⁵, R⁶, A, R′, R″, Cycle2, and Aryl3 are as defined herein;
   G is selected from the group consisting of OH and Cl;
when G is OH, step b) comprises contacting the compound according to Formula (XI) with R¹-A-C(O)-G in the presence of a coupling reagent; and
wherein the coupling reagent more particularly is 2-(1H-benzotriazol-1-yl)-1,1,3,3-tetramethyluronium hexafluorophosphate (HBTU).

In another embodiment, the application relates to a method for the preparation of a compound as described herein, wherein R⁵ is other than hydrogen. The method comprises the step of providing a compound according to Formula (XI):

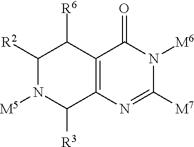

Formula (XI) or a tautomer thereof; wherein
   M⁵ is selected from the group consisting of tert-butyloxycarbonyl, hydrogen, and R¹-A-C(O);
   M⁶ is R⁵ is other than hydrogen; and
   M⁷ is selected from the group consisting of sulfhydryl, —SCH₃, —Cl, —S(O)CH₃, and R⁴;
   wherein when M⁵ is R¹-A-C(O)—, then M⁷ is not R⁴;
   wherein the method further comprises at least one of steps a) to f):
a) contacting the compound according to Formula (XI) with a strong acid, more particularly hydrochloric acid or trifluoroacetic acid;
b) contacting the compound according to Formula (XI) with R¹-A-C(O)-G, more particularly in the presence of a non-nucleophilic base;
c) contacting the compound according to Formula (XI) with a methylating agent, more particularly methyl iodide;
d) contacting the compound according to Formula (XI) with an oxidizing agent, more particularly meta-chloroperoxybenzoic acid;
e) contacting the compound according to Formula (XI) with a chlorinating agent, more particularly thiophosgene;
f) contacting the compound according to Formula (XI) with one of the reagents consisting of R′—S—H, R′—O—H and R″NH₂, more particularly in the absence or in the presence of a non-nucleophilic base;
   wherein when M⁵ is tert-butyloxycarbonyl, the method comprises at least steps a) and b);
   wherein when M⁵ is hydrogen, the method comprises at least step b);
   wherein when M⁷ is sulfhydryl the method comprises at least steps c), d) and f); or at least steps e) and f);
   wherein when M⁷ is —SCH₃, the method comprises at least steps d) and f);
   wherein when M⁷ is —Cl or —S(O)CH₃, the method comprises at least step f);
   wherein when the method comprises steps c) and/or d), the method does not comprise step e); and wherein:
   R¹, R², R³, R⁴, R⁵, R⁶, A, R′, R″, Cycle2, and Aryl3 are as defined herein and with the proviso that R⁵ is other than hydrogen;
   G is selected from the group consisting of OH and Cl;
when G is OH, step b) comprises contacting the compound according to Formula (XI) with R¹-A-C(O)-G in the presence of a coupling reagent; and
wherein the coupling reagent more particularly is 2-(1H-benzotriazol-1-yl)-1,1,3,3-tetramethyluronium hexafluorophosphate (HBTU).

In yet another embodiment, the application relates to a process for the preparation of a compound as described herein. The process comprises the steps of:
a) contacting a compound of Formula (XII) with R⁵—NH₂, wherein Formula (XII) is

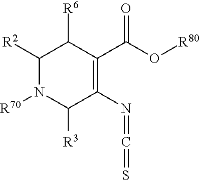

Formula (XII), to provide the compound of Formula (XIII):

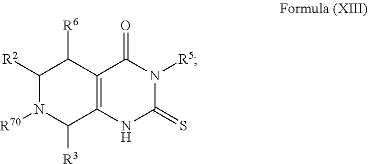

b) contacting the compound of Formula (XIII) with a methylating agent, more particularly methyl iodide, to form a compound according to Formula (XIV):

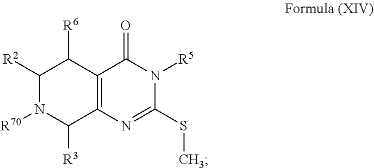

c) contacting the compound of Formula (XIV) with an oxidizing agent, more particularly meta-chloroperoxybenzoic acid, to form a compound of Formula (XV):

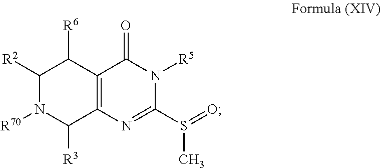

d) contacting the compound of Formula (XV) with R″NH₂, more particularly in the presence of a non-nucleophilic base, to form a compound of Formula (XVI):

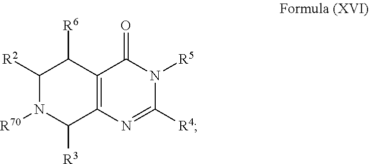

e) contacting the compound of Formula (XVI) with a strong acid, more particularly hydrochloric acid or trifluoroacetic acid, to form a compound of Formula (XVII):

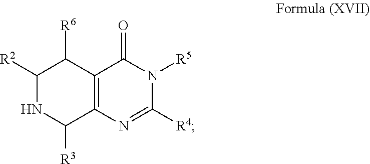

f) contacting the compound of Formula (XVII) with R¹-A-C(O)-G;
wherein:
R¹, R², R³, R⁴, R⁵, R⁶, A, and R″ are as defined herein;
R⁷⁰ is tert-butyloxycarbonyl;
R⁸⁰ is C₁₋₄alkyl; and
G is selected from the group consisting of OH and Cl,
when G is OH, step f) comprises contacting the compound according to Formula (XVII) with R¹-A-C(O)-G in the presence of a coupling reagent, more particularly in the presence of a non-nucleophilic base; and
wherein the coupling reagent more particularly is 2-(1H-benzotriazol-1-yl)-1,1,3,3-tetramethyluronium hexafluorophosphate (HBTU).

In an embodiment, the application relates to a method for the preparation of a compound as described herein. The method comprises the step of providing a compound according to Formula (XI):

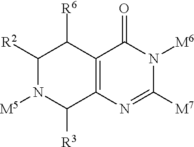

Formula (XI) or a tautomer thereof; wherein
   M⁵ is selected from the group consisting of tert-butyloxycarbonyl, hydrogen, and R¹-A-C(O);
   M⁶ is selected from the group consisting of tert-butyloxycarbonyl, and H; and
   M⁷ is selected from the group consisting of sulfhydryl, —SCH₃, —Cl, —S(O)CH₃, and R⁴;
   wherein when M⁵ is R¹-A-C(O)— and M⁶ is H, then M⁷ is not R⁴;
   wherein when M⁵ and M⁶ are hydrogen, then M⁷ is not —NH₂;
   wherein the method further comprises at least one of steps a) to h):
a) contacting the compound according to Formula (XI) with a strong acid, more particularly hydrochloric acid or trifluoroacetic acid;
b) contacting the compound according to Formula (XI) with R¹-A-C(O)-G, more particularly in the presence of a non-nucleophilic base;
c) contacting the compound according to Formula (XI) with M¹⁰-H^{A}, more particularly in the presence of Cs₂CO₃, wherein M¹⁰ is selected from the group consisting of C₁₋₆alkyl, C₁₋₃alkenyl, and Cycle2; wherein C₁₋₆alkyl is optionally substituted with phenyl, methoxyphenyl, OC₁₋₆alkyl, NHSO₂CH₃, and C₃₋₆cycloalkyl optionally containing an oxygen atom;
   and wherein H^{A} is a halogen, more particularly Br;
d) contacting the compound according to Formula (XI) with Aryl3-B(OH)₂, more particularly in the presence of copper(II) acetate;
e) contacting the compound according to Formula (XI) with a methylating agent, more particularly methyl iodide;
f) contacting the compound according to Formula (XI) with an oxidizing agent, more particularly meta-chloroperoxybenzoic acid;
g) contacting the compound according to Formula (XI) with a chlorinating agent, more particularly thiophosgene;
h) contacting the compound according to Formula (XI) with one of the reagents consisting or R′S—H, R′O—H and R″NH₂, more particularly in the absence or in the presence of a non-nucleophilic base;
   wherein when M⁵ is tert-butyloxycarbonyl, the method comprises at least steps a) and b);
   wherein when M⁵ is hydrogen, the method comprises at least step b);
   wherein when M⁶ is hydrogen, the method optionally comprises at least step c) or step d);
wherein when M⁶ is tert-butyloxycarbonyl, the method comprises at least steps a) and c) or at least steps a) and d);
   wherein when M⁷ is sulfhydryl the method comprises at least steps e), f) and h); or at least steps g) and h);
   wherein when M⁷ is —SCH₃, the method comprises at least steps f) and h);
   wherein when M⁷ is —Cl or —S(O)CH₃, the method comprises at least step h);
   wherein when the method comprises steps e) and/or f), the method does not comprise step g);
   wherein when the method comprises step c), the method does not comprise step d);
   and wherein:
      R¹, R², R³, R⁴, R⁵, R⁶, A, R″, Cycle2, and Aryl3 are as defined herein;
      G is selected from the group consisting of OH and Cl;
when G is OH, step b) comprises contacting the compound according to Formula (XI) with R¹-A-C(O)-G in the presence of a coupling reagent; and
wherein the coupling reagent more particularly is 2-(1H-benzotriazol-1-yl)-1,1,3,3-tetramethyluronium hexafluorophosphate (HBTU).

In an embodiment, the application relates to a method for the preparation of a compound as described herein. The method comprises the step of providing a compound according to Formula (XI):

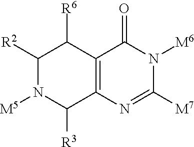

Formula (XI) or a tautomer thereof;
wherein
   M⁵ is selected from the group consisting of tert-butyloxycarbonyl, hydrogen, and R¹-A-C(O);

### M⁶ is H; and

M⁷ is selected from the group consisting of sulfhydryl, —SCH₃, —Cl, —S(O)CH₃, and R⁴;
wherein when M⁵ is R¹-A-C(O)— and M⁶ is H, then M⁷ is not R⁴;
wherein when M⁵ and M⁶ are hydrogen, then M⁷ is not —NH₂;
wherein the method further comprises at least the following step c):
c) contacting the compound according to Formula (XI) with M¹⁰-H^{A}, more particularly in the presence of Cs₂CO₃, wherein M¹⁰ is selected from the group consisting of C₁₋₆alkyl, C₁₋₃alkenyl, and Cycle2; wherein C₁₋₆alkyl is optionally substituted with phenyl, methoxyphenyl, OC₁₋₆alkyl, NHSO₂CH₃, and C₃₋₆cycloalkyl optionally containing an oxygen atom;
   and wherein H^{A} is a halogen, more particularly Br;
      wherein the method optionally further comprises at least one step from among steps a), b), d), e), f), g) and h):
a) contacting the compound according to Formula (XI) with a strong acid, more particularly hydrochloric acid or trifluoroacetic acid;
b) contacting the compound according to Formula (XI) with R¹-A-C(O)-G, more particularly in the presence of a non-nucleophilic base;
d) contacting the compound according to Formula (XI) with Aryl3-B(OH)₂, more particularly in the presence of copper(II) acetate;
e) contacting the compound according to Formula (XI) with a methylating agent, more particularly methyl iodide;
f) contacting the compound according to Formula (XI) with an oxidizing agent, more particularly meta-chloroperoxybenzoic acid;
g) contacting the compound according to Formula (XI) with a chlorinating agent, more particularly thiophosgene;
h) contacting the compound according to Formula (XI) with one of the reagents consisting of R′—S—H, R′—O—H and R′R″N—H, more particularly in the absence or in the presence of a non-nucleophilic base;
   wherein when M⁵ is tert-butyloxycarbonyl, the method further comprises the steps a) and b);
   wherein when M⁵ is hydrogen, the method further comprises step b);
   wherein when M⁷ is sulfhydryl the method further comprises steps e), f) and h); or steps g) and h);
   wherein when M⁷ is —SCH₃, the method further comprises steps f) and h);
   wherein when M⁷ is —Cl or —S(O)CH₃, the method further comprises step h);
   wherein when the method further comprises steps e) and/or f), the method does not comprise step g);
   and wherein:
   R¹, R², R³, R⁴, R⁵, R⁶, A, R′, R″, Cycle2, and Aryl3 are as defined herein;
   G is selected from the group consisting of OH and Cl;
when G is OH, step b) comprises contacting the compound according to Formula (XI) with R¹-A-C(O)-G in the presence of a coupling reagent; and
wherein the coupling reagent more particularly is 2-(1H-benzotriazol-1-yl)-1,1,3,3-tetramethyluronium hexafluorophosphate (HBTU).

In another embodiment, the application relates to a method for the preparation of a compound as described herein. The method comprises the step of providing a compound according to Formula (XI):

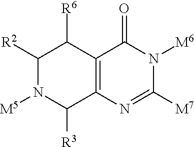

Formula (XI) or a tautomer thereof; wherein
   M⁵ is selected from the group consisting of tert-butyloxycarbonyl, hydrogen, and R¹-A-C(O);
   M⁶ is selected from the group consisting of tert-butyloxycarbonyl, and H; and
   M⁷ is selected from the group consisting of sulfhydryl, —SCH₃, —Cl, —S(O)CH₃, and R⁴;
   wherein when M⁵ is R¹-A-C(O)— and M⁶ is H, then M⁷ is not R⁴;
   wherein when M⁵ and M⁶ are hydrogen, then M⁷ is not —NH₂;
   wherein the method further comprises at least step c):
c) contacting the compound according to Formula (XI) with M¹⁰-H^{A}, more particularly in the presence of Cs₂CO₃, wherein M¹⁰ is selected from the group consisting of C₁₋₆alkyl, C₁₋₃alkenyl, and Cycle2; wherein C₁₋₆alkyl is optionally substituted with phenyl, methoxyphenyl, OC₁₋₆alkyl, NHSO₂CH₃, and C₃₋₆cycloalkyl optionally containing an oxygen atom;
   and wherein H^{A} is a halogen, more particularly Br;
      and wherein the method optionally further comprises at least one step from among steps a), b), e), f), g) and h):
a) contacting the compound according to Formula (XI) with a strong acid, more particularly hydrochloric acid or trifluoroacetic acid;
b) contacting the compound according to Formula (XI) with R¹-A-C(O)-G, more particularly in the presence of a non-nucleophilic base;
e) contacting the compound according to Formula (XI) with a methylating agent, more particularly methyl iodide;
f) contacting the compound according to Formula (XI) with an oxidizing agent, more particularly meta-chloroperoxybenzoic acid;
g) contacting the compound according to Formula (XI) with a chlorinating agent, more particularly thiophosgene;
h) contacting the compound according to Formula (XI) with H—X—R′, more particularly in the absence or in the presence of a non-nucleophilic base;
   wherein when M⁵ is tert-butyloxycarbonyl, the method further comprises the steps a) and b);
   wherein when M⁵ is hydrogen, the method further comprises step b);
   wherein when M⁶ is tert-butyloxycarbonyl, the method further comprises steps a) and c);
   wherein when M⁷ is sulfhydryl the method further comprises steps e), f) and h); or steps g) and h);
   wherein when M⁷ is —SCH₃, the method further comprises steps f) and h);
   wherein when M⁷ is —Cl or —S(O)CH₃, the method further comprises step h);
   wherein when the method further comprises steps e) and/or f), the method does not comprise step g);
   and wherein:
      R¹, R², R³, R⁴, R⁵, R⁶, A, X, R′, Cycle2, and Aryl3 are as defined herein;
      G is selected from the group consisting of OH and Cl;
when G is OH, step b) comprises contacting the compound according to Formula (XI) with R¹-A-C(O)-G in the presence of a coupling reagent; and
wherein the coupling reagent more particularly is 2-(1H-benzotriazol-1-yl)-1,1,3,3-tetramethyluronium hexafluorophosphate (HBTU).

In an embodiment, the application relates to a method for the preparation of a compound as described herein. The method comprises the step of providing a compound according to Formula (XI):

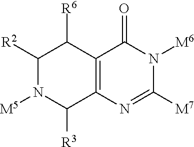

Formula (XI) or a tautomer thereof;
wherein
   M⁵ is selected from the group consisting of tert-butyloxycarbonyl, hydrogen, and R¹-A-C(O);

### M⁶ is H; and

M⁷ is selected from the group consisting of sulfhydryl, —SCH₃, —Cl, —S(O)CH₃, and R⁴;
wherein when M⁵ is R¹-A-C(O)— and M⁶ is H, then M⁷ is not R⁴;
wherein when M⁵ and M⁶ are hydrogen, then M⁷ is not —NH₂;
wherein the method further comprises step d):
d) contacting the compound according to Formula (XI) with Aryl3-B(OH)₂, more particularly in the presence of copper(II) acetate;
   wherein the method further optionally comprises at least one step from among steps a), b), c), e), f), g) and h),
a) contacting the compound according to Formula (XI) with a strong acid, more particularly hydrochloric acid or trifluoroacetic acid;
b) contacting the compound according to Formula (XI) with R¹-A-C(O)-G, more particularly in the presence of a non-nucleophilic base;
c) contacting the compound according to Formula (XI) with M¹⁰-H^{A}, more particularly in the presence of Cs₂CO₃, wherein M¹⁰ is selected from the group consisting of C₁₋₆alkyl, C₁₋₃alkenyl, and Cycle2; wherein C₁₋₆alkyl is optionally substituted with phenyl, methoxyphenyl, OC₁₋₆alkyl, NHSO₂CH₃, and C₃₋₆cycloalkyl optionally containing an oxygen atom;
   and wherein H^{A} is a halogen, more particularly Br;
e) contacting the compound according to Formula (XI) with a methylating agent, more particularly methyl iodide;
f) contacting the compound according to Formula (XI) with an oxidizing agent, more particularly meta-chloroperoxybenzoic acid;
g) contacting the compound according to Formula (XI) with a chlorinating agent, more particularly thiophosgene;
h) contacting the compound according to Formula (XI) with one of the reagents consisting of R′—S—H, R′—O—H and R′R″N—H, more particularly in the absence or in the presence of a non-nucleophilic base;
   wherein when M⁵ is tert-butyloxycarbonyl, the method further comprises the steps a) and b);
   wherein when M⁵ is hydrogen, the method further comprises step b);
   wherein when M⁷ is sulfhydryl the method further comprises steps e), f) and h); or steps g) and h);
   wherein when M⁷ is —SCH₃, the method further comprises steps f) and h);
   wherein when M⁷ is —Cl or —S(O)CH₃, the method further comprises step h); wherein when the method further comprises steps e) and/or f), the method does not comprise step g);
   and wherein:
   R¹, R², R³, R⁴, R⁵, R⁶, A, X, R′, R″, Cycle2, and Aryl3 are as defined herein;
   G is selected from the group consisting of OH and Cl;
when G is OH, step b) comprises contacting the compound according to Formula (XI) with R¹-A-C(O)-G in the presence of a coupling reagent; and
wherein the coupling reagent more particularly is 2-(1H-benzotriazol-1-yl)-1,1,3,3-tetramethyluronium hexafluorophosphate (HBTU).

In an embodiment, the application relates to a method for the preparation of a compound as described herein. The method comprises the step of providing a compound according to Formula (XI):

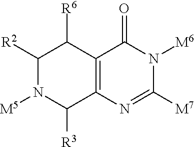

Formula (XI) or a tautomer thereof; wherein
   M⁵ is selected from the group consisting of tert-butyloxycarbonyl, hydrogen, and R¹-A-C(O);
   M⁶ is selected from the group consisting of tert-butyloxycarbonyl, and H; and
   M⁷ is selected from the group consisting of sulfhydryl, —SCH₃, —Cl, —S(O)CH₃, and R⁴;
   wherein when M⁵ is R¹-A-C(O)— and M⁶ is H, then M⁷ is not R⁴;
   wherein when M⁵ and M⁶ are hydrogen, then M⁷ is not —NH₂;
   wherein the method further comprises step d):
d) contacting the compound according to Formula (XI) with Aryl3-B(OH)₂, more particularly in the presence of copper(II) acetate;
   wherein the method optionally further comprises at least one step from among steps a), b), c), e), f), g) and h):
a) contacting the compound according to Formula (XI) with a strong acid, more particularly hydrochloric acid or trifluoroacetic acid;
b) contacting the compound according to Formula (XI) with R¹-A-C(O)-G, more particularly in the presence of a non-nucleophilic base;
c) contacting the compound according to Formula (XI) with M¹⁰-H^{A}, more particularly in the presence of Cs₂CO₃, wherein M¹⁰ is selected from the group consisting of C₁₋₆alkyl, C₁₋₃alkenyl, and Cycle2; wherein C₁₋₆alkyl is optionally substituted with phenyl, methoxyphenyl, OC₁₋₆alkyl, NHSO₂CH₃, and C₃₋₆cycloalkyl optionally containing an oxygen atom;
   and wherein H^{A} is a halogen, more particularly Br;
e) contacting the compound according to Formula (XI) with a methylating agent, more particularly methyl iodide;
f) contacting the compound according to Formula (XI) with an oxidizing agent, more particularly meta-chloroperoxybenzoic acid;
g) contacting the compound according to Formula (XI) with a chlorinating agent, more particularly thiophosgene;
h) contacting the compound according to Formula (XI) with H—X—R′, more particularly in the absence or in the presence of a non-nucleophilic base;
   wherein when M⁵ is tert-butyloxycarbonyl, the method further comprises the steps a) and b);
   wherein when M⁵ is hydrogen, the method further comprises step b);
   wherein when M⁶ is hydrogen, the method further optionally further comprises step c);
   wherein when M⁶ is tert-butyloxycarbonyl, the method further comprises steps a) and c) or step a);
   wherein when M⁷ is sulfhydryl the method further comprises steps e), f) and h); or steps g) and h);
   wherein when M⁷ is —SCH₃, the method further comprises steps f) and h);
   wherein when M⁷ is —Cl or —S(O)CH₃, the method further comprises step h);
   wherein when the method further comprises steps e) and/or f), the method does not comprise step g);
   and wherein:
   R¹, R², R³, R⁴, R⁵, R⁶, A, X, R′, Cycle2, and Aryl3 are as defined herein;
   G is selected from the group consisting of OH and Cl;
when G is OH, step b) comprises contacting the compound according to Formula (XI) with R¹-A-C(O)-G in the presence of a coupling reagent; and
wherein the coupling reagent more particularly is 2-(1H-benzotriazol-1-yl)-1,1,3,3-tetramethyluronium hexafluorophosphate (HBTU).

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The term “comprising”, which is synonymous with “including” or “containing”, is open-ended, and does not exclude additional, unrecited element(s), ingredient(s) or method step(s), whereas the term “consisting of” is a closed term, which excludes any additional element, step, or ingredient which is not explicitly recited.

The term “essentially consisting of” is a partially open term, which does not exclude additional, unrecited element(s), step(s), or ingredient(s), as long as these additional element(s), step(s) or ingredient(s) do not materially affect the basic and novel properties of the subject matter of the application.

The term “comprising” (or “comprise(s)”) hence includes the term “consisting of” (“consist(s) of”), as well as the term “essentially consisting of” (“essentially consist(s) of”). Accordingly, the term “comprising” (or “comprise(s)”) is, in the present application, meant as more particularly encompassing the term “consisting of” (“consist(s) of”), and the term “essentially consisting of” (“essentially consist(s) of”).

In an attempt to help the reader of the present application, the description has been separated in various paragraphs or sections. These separations should not be considered as disconnecting the substance of a paragraph or section from the substance of another paragraph or section. To the contrary, the present description encompasses all the combinations of the various sections, paragraphs and sentences that can be contemplated.

Each of the relevant disclosures of all references cited herein is specifically incorporated by reference. The following examples are offered by way of illustration, and not by way of limitation.

### Examples

### 1. General Information

### 1.1. General Procedure for LCMS Methods

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software. Compounds are described by their experimental retention times (Rt) and ions. If not specified differently in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, etc. . . . ). All results were obtained with experimental uncertainties that are commonly associated with the method used.

Hereinafter, “SQD” means Single Quadrupole Detector, “MSD” Mass Selective Detector, “RT” room temperature, “BEH” bridged ethylsiloxane/silica hybrid, “DAD” Diode Array Detector, “HSS” High Strength silica., “Q-Tof” Quadrupole Time-of-flight mass spectrometers, “CLND”, ChemiLuminescent Nitrogen Detector, “ELSD” Evaporative Light Scanning Detector,

### LCMS Methods

(Flow expressed in mL/min; column temperature (T) in ° C.; Run time in minutes).

| Method | | | | | Flow | Run |
|---|---|---|---|---|---|---|
| code | Instrument | Column | Mobile phase | Gradient | Col T | time |
| A | Waters: | Waters: | A: 10 mM | From 100% A | 0.7 | 3.5 |
| | Acquity^{R} | HSS T3 | CH₃COONH₄ | to 5% A in | 55 | |
| | UPLC^{R}- | (1.8 μm, | in 95% H₂O + | 2.10 min, to | | |
| | DAD and | 2.1*100 mm) | 5% CH₃CN | 0% A in 0.90 | | |
| | SQD | | B: CH₃CN | min, to 5% A | | |
| | | | | in 0.5 min | | |
| B | Waters: | Waters: | A: 10 mM | From 100% A | 0.7 | 3.5 |
| | Acquity^{R} | HSS T3 | CH₃COONH₄ | to 5% A in | 55 | |
| | UPLC^{R}- | (1.8 μm, | in 95% H₂O + | 2.10 min, to | | |
| | DAD, | 2.1*100 mm) | 5% CH₃CN | 0% A in 0.90 | | |
| | SQD and | | B: CH₃CN | min, to 5% A | | |
| | ELSD | | | in 0.5 min | | |
| C | Waters: | Waters: | A: 10 mM | From 95% A | 0.8 | 2 |
| | AcquityR | BEH C18 | CH₃COONH₄ | to 5% A in | 55 | |
| | UPLCR- | (1.7 μm, | in 95% H₂O + | 1.3 min, held | | |
| | DAD and | 2.1*50 mm) | 5% CH₃CN | for 0.7 min. | | |
| | SQD | | B: CH₃CN | | | |
| D | Waters: | Waters: | A: 10 mM | From 100% | 0.6 | 3.5 |
| | Acquity^{R} | BEH | CH₃COONH₄ | to 5% A in | 55 | |
| | UPLC^{R}- | (1.8 μm, | in 95% H₂O + | 2.10 min, to | | |
| | DAD and | 2.1*100 mm) | 5% CH₃CN | 0% A in 0.90 | | |
| | SQD | | B: CH₃CN | min, to 5% A | | |
| | | | | in 0.5 min | | |
| E | Waters: | Waters: | A: 0.1% | From 95% A | 0.7 | 3 |
| | Acquity^{R} | BEH C18 | HCOOH + | to 0% A in | 55 | |
| | UPLC^{R}- | (1,7 μm, | 5% CH₃OH | 2.50 min, to | | |
| | DAD- | 2.1*50 mm) | in H₂O | 5% A in 0.5 | | |
| | SQD and | | B: CH₃CN | min | | |
| | ELSD | | | | | |
| F | Waters: | Waters: | A: 0.1% | From 100% A | 0.6 | 3.5 |
| | Acquity^{R} | BEH | NH₄HCO₃ in | to 5% A in | 55 | |
| | UPLC^{R}- | (1.8 μm, | H₂O | 2.10 min, to | | |
| | DAD, SQD | 2.1*100 mm) | B: CH₃CN | 0% A in 0.90 | | |
| | | | | min, to 5% A | | |
| | | | | in 0.5 min | | |
| G | Waters: | Waters: | A: 0.1% | From 100% A | 0.7 | 3.5 |
| | AcquityR | BEH | NH₄HCO₃ in | to 5% A in | 55 | |
| | UPLCR- | (1.8 μm, | 95% H₂O + | 2.10 min, to | | |
| | DAD and | 2.1*100 mm) | 5% CH₃CN | 0% A in 0.90 | | |
| | SQD | | B: CH₃CN | min, to 5% A | | |
| | | | | in 0.5 min | | |
| H | Waters: | Waters: | A: 10 mM | From 100% A | 0.7 | 3.5 |
| | AcquityR | BEH | CH₃COONH₄ | to 5% A in | 55 | |
| | UPLCR- | (1.8 μm, | in 95% H₂O + | 2.10 min, to | | |
| | DAD and | 2.1*100 mm) | 5% CH₃CN | 0% A in 0.90 | | |
| | SQD | | B: CH₃CN | min, to 5% A | | |
| | | | | in 0.5 min | | |
| I | Waters: | Waters: | A: 10 mM | From 100% A | 0.6 | 3.5 |
| | Acquity^{R} | BEH | CH₃COONH₄ | to 5% A in | 55 | |
| | UPLC^{R}- | (1.8 μm, | in 95% H₂O + | 2.10 min, to | | |
| | DAD and | 2.1*100 mm) | 5% CH₃CN | 0% A in 0.90 | | |
| | SQD | | B: CH₃CN | min, to 5% A | | |
| | | | | in 0.5 min | | |
| J | Thermo- | Agilent: | A: HCO₂H | 98% A for 2 | 1 | 18.4 |
| | scientific | Poroshell | 0.1% in | min, to 0% A | 30 | |
| | Ultimate | EC-C18 | water/ | in 10 min, | | |
| | 3000 DAD | (4 μm, | B: HCOOH | held for 3.4 | | |
| | and Brucker | 4.6 × 100 mm) | 0.05% in | min, back to | | |
| | HCT ultra | | CH₃CN | 98% Ain 1.3 | | |
| | | | | min, held for | | |
| | | | | 1.7 min | | |
| K | Waters: | Waters: | A: 10 mM | From 100% A | 0.6 | 3.5 |
| | AcquityR | HSS T3 | CH₃COONH₄ | to 5% A in | 55 | |
| | UPLCR- | (1.8 μm, | in 95% H₂O + | 2.10 min, to | | |
| | DAD, SQD | 2.1*100 mm) | 5% CH₃CN | 0% A in | | |
| | and ELSD | | B: CH₃CN | 0.90 min, to | | |
| | | | | 5% A in | | |
| | | | | 0.5 min | | |
| L | Waters: | Waters: | A: 0.1% | From 100% A | 0.6 | 5.5 |
| | AcquityR | BEH | NH₄HCO₃ | to 5% A in | 55 | |
| | UPLCR- | (1.8 μm, | in 95% H₂O + | 2.10 min, | | |
| | DAD and | 2.1*100 mm) | 5% CH₃CN | to 0% A in | | |
| | SQD | | B: MeOH | 0.9 min, | | |
| | | | | to 5% A in | | |
| | | | | 0.5 min | | |
| M | Waters: | Atlantis T3 | A: | 100% B to | 1.5 | 13.5 |
| | Alliance ®- | column | 70% CH3OH, | 5% B in 9 min, | 45 | |
| | DAD-ZMD | (5 μm, 4.6 × | 30% H2O | hold 3.0 min | | |
| | and CLND | 100 mm) | B: 0.1 formic acid | to 100% B in | | |
| | 8060 Antek | | in H2O/methanol | 1 min and | | |
| | | | 95/5 | hold 0.5 min | | |
| N | Agilent | SB-C18 | A-acetonitrile: | 0.01 min -99% B | 3 | 5.92 |
| | Poroshell | 4.6 × 30 mm | water (99:1%), | 1.5 min - 0% B | 60 | |
| | 120 | 2.7 μm | 0.1% formic acid | 2.2 min - 0% B | | |
| | | with UHPLC | B-water | 2.21 min - 99% B | | |
| | | Guard Infinity | (0.1% formic acid) | | | |
| | | Lab Poroshell | | | | |
| | | 120 SB-C18 | | | | |
| | | 4.6 × 5 mm | | | | |
| | | 2.7 μm | | | | |

### 1.2. General Procedure for SFC Methods

### SFC Methods

(Flow expressed in mL/min; column temperature (T) in OC; Run time in minutes).

| Method | | | | Flow | Run time |
|---|---|---|---|---|---|
| code | column | mobile phase | gradient | Col T | BPR |
| SFC_A | Daicel ChiralpakR | A: CO₂ | 10%-50% B | 2.5 | 9.5 |
| | AD-H column (3.0 | B: EtOH + 0.2% | in 6 min, | 40 | 110 |
| | μm, 150 × 4.6 mm) | i-PrNH₂ | hold 3.5 min | | |
| SFC_B | Daicel ChiralpakR | A: CO₂ | 10%-50% B | 2.5 | 9.5 |
| | AD-H column (3.0 | B: i-PrOH + 0.2% | in 6 min, | 40 | 110 |
| | μm, 150 × 4.6 mm) | i-PrNH₂ | hold 3.5 min | | |
| SFC_C | Daicel ChiralpakR | A: CO₂ | 10%-50% B | 2.5 | 9.5 |
| | IG3 column (3.0 | B: EtOH + 0.2% | in 6 min, | 40 | 130 |
| | μm, 150 × 4.6 mm) | i-PrNH₂ | hold 3.5 min | | |
| SFC_D | Daicel ChiralpakR | A: CO₂ | 10%-50% B | 2.5 | 9.5 |
| | IC3 column (3.0 | B: EtOH + 0.2% | in 6 min, | 40 | 130 |
| | μm, 150 × 4.6 mm) | i-PrNH₂ | hold 3.5 min | | |
| SFC_E | Daicel ChiralpakR | A: CO₂ | 10%-50% B | 2.5 | 9.5 |
| | OJ3 column (3.0 | B: EtOH + 0.2% | in 6 min, | 40 | 130 |
| | μm, 150 × 4.6 mm) | i-PrNH₂ | hold 3.5 min | | |
| SFC_F | Daicel Chiralpak ® | A: CO₂ | 10%-50% B | 2.5 | 9.5 |
| | AS3 column (3.0 | B: MeOH + 0.2% | in 6 min, | 40 | 130 |
| | μm, 150 × 4.6 mm) | iPrNH2 | hold 3.5 min | | |
| SFC_G | Daicel Chiralpak ® | A: CO₂ | 10%-50% B | 2.5 | 9.5 |
| | AS3 column (3.0 | B: EtOH + 0.2% | in 6 min, | 40 | 130 |
| | μm, 150 × 4.6 mm) | iPrNH₂ | hold 3.5 min | | |
| SFC_H | Daicel Chiralpak ® | A: CO₂ | 10%-50% B | 2.5 | 9.5 |
| | OJ3 column (3.0 | B: MeOH + 0.2% | in 6 min, | 40 | 130 |
| | μm, 150 × 4.6 mm) | iPrNH₂ | hold 3.5 min | | |
| SFC_I | Daicel Chiralpak ® | A: CO₂ | 10%-50% B | 2.5 | 9.5 |
| | IC3 column (3.0 | B: MeOH + 0.2% | in 6 min, | 40 | 130 |
| | μm, 150 × 4.6 mm) | iPrNH₂ | hold 3.5 min | | |
| SFC_J | Daicel Chiralpak ® | A: CO₂ | 10%-50% B | 2.5 | 9.5 |
| | AD3 column (3.0 | B: EtOH-iPrOH | in 6 min, | 40 | 130 |
| | μm, 150 × 4.6 mm) | (50-50) + 0.2% | hold 3.5 min | | |
| | | iPrNH₂ | | | |
| SFC_K | Daicel Chiralpak ® | A: CO2 | 10%-50% B | 2.5 | 9.5 |
| | ID3 column (3.0 | B: iPrOH + 0.2% | in 6 min, | 40 | 130 |
| | μm, 150 × 4.6 mm) | iPrNH2 | hold 3.5 min | | |
| SFC_L | Daicel Chiralpak ® | A: CO2 | 10%-50% B | 2.5 | 9.5 |
| | ID3 column (3.0 | B: MeOH + 0.2% | in 6 min, | 40 | 130 |
| | μm, 150 × 4.6 mm) | iPrNH2 | hold 3.5 min | | |
| SFC_M | Daicel Chiralpak ® | A: CO2 | 10%-50% B | 2.5 | 9.5 |
| | IC3 column (3.0 | B: EtOH + 0.2% | in 6 min, | 40 | 130 |
| | μm, 150 × 4.6 mm) | iPrNH2 | hold 3.5 min | | |
| SFC_N | Daicel Chiralpak ® | A: CO2 | 50% B in 6 | 2.5 | 9.5 |
| | IC3 column (3.0 | B: EtOH + 0.2% | min, hold | 40 | 130 |
| | μm, 150 × 4.6 mm) | iPrNH2 | 9.5 min | | |
| SFC_O | Daicel Chiralpak ® | A: CO2 | 10%-50% B | 2.5 | 9.5 |
| | AD3 column (3.0 | B: MeOH + 0.2% | in 6 min, | 40 | 130 |
| | μm, 150 × 4.6 mm) | iPrNH2 | hold 3.5 min | | |
| SFC_P | Daicel Chiralpak ® | A: CO2 | 10%-50% B | 2.5 | 9.5 |
| | OJ3 column (3.0 | B: iPrOH + 0.2% | in 6 min, | 40 | 130 |
| | μm, 150 × 4.6 mm) | iPrNH2 | hold 3.5 min | | |
| SFC_Q | Daicel Chiralpak ® | A: CO2 | 10%-50% B | 2.5 | 9.5 |
| | OJ3 column (3.0 | B: iPrOH + 0.2% | in 6 min, | 40 | 130 |
| | μm, 150 × 4.6 mm) | iPrNH2 | hold 3.5 min | | |
| SFC_R | Daicel Chiralpak ® | A: CO2 | 10%-50% B | 2.5 | 9.5 |
| | IC3 column (3.0 | B: EtOH-iPrOH | in 6 min, | 40 | 130 |
| | μm, 150 × 4.6 mm) | (50-50) + 0.2% | hold 3.5 min | | |
| | | iPrNH2 | | | |

### 1.3. NMR Analysis

¹H NMR spectra were recorded on 1) a Bruker DPX 400 MHz spectrometer or 2) a Bruker Avance 400 MHz spectrometer or 3) a Bruker Avance III 400 MHz spectrometer or 4) a Bruker Avance 600 MHz spectrometer.

NMR spectra were recorded at ambient temperature unless otherwise stated. Data are reported as follow: chemical shift in parts per million (ppm) relative to TMS (b=0 ppm) on the scale, integration, multiplicity (s=singulet, d=doublet, t=triplet, q=quartet, quin=quintuplet, sext=sextuplet, sept=septuplet, m=multiplet, b=broad, or a combination of these), coupling constant(s) J in Hertz (Hz).

### 1.3. Separation of the Rotamers, Diastereoisomers and Enantiomers

Rotamers, diasteroisomers and enantiomers have been purified by preparative SFC and/or by preparative HPLC. The conditions and the compounds that have been purified are listed below.

### Description of the Methods

| Method | Conditions |
|---|---|
| A | Preparative SFC (Stationary phase: Chiralpak Diacel AS 20 × 250 mm, |
| | mobile phase: CO₂, EtOH + 0.4% i-PrNH₂) |
| B | Preparative SFC (Stationary phase: Chiralcel Diacel OJ 20 × 250 mm, |
| | mobile phase: CO₂, EtOH + 0.4% i-PrNH₂). |
| C | Preparative SFC (stationary phase: Chiralpak Daicel ID 20 × 250 mm, |
| | mobile phase: CO₂, EtOH + 0.4% i-PrNH₂). |
| D | Preparative HPLC (stationary phase: Chiralcel Diacel OJ 20 × 250 mm, |
| | mobile phase: CO₂, EtOH + 0.4% i-PrNH₂). |
| E | Preparative HPLC (stationary phase: Chiralpak Diacel AD 20 × 250 mm, |
| | mobile phase: CO₂, EtOH + 0.4% i-PrNH₂). |
| F | Preparative SFC (stationary phase: Chiralpak Diacel AD 20 × 250 mm, |
| | mobile phase: CO₂, EtOH + 0.4% i-PrNH₂). |
| G | Preparative SFC (stationary phase: Chiralpak Daicel IC 20 × 250 mm, |
| | mobile phase: CO₂, EtOH + 0.4% i-PrNH₂). |
| H | Preparative SFC (stationary phase: Chiralpak Diacel AD 20 × 250 mm, |
| | mobile phase: CO₂, EtOH:i-PrOH (50:50) + 0.4% i-PrNH₂). |
| I | Preparative SFC (stationary phase: Chiralcel Diacel OJ 20 × 250 mm, |
| | mobile phase: CO₂, EtOH:i-PrOH (50:50) + 0.4% i-PrNH₂). |

### Separation of the Compounds

| Compounds | Method |
|---|---|
| C26a and C26b | A |
| C36Aa, C36Bb, C36Ab and C36Ba | A |
| C62A, C62Ba and C62Bb | A, then B |
| C63A and C63B | C |
| C100A and C100B | D |
| C101a and C101b | E |
| C109A and C109B | A |
| C110A and C110B | F |
| C154A and C154B | B |
| C158A and C158B | B |
| C256 and C257 | A |
| C282 and C283 | A |
| C285 and C286 | B |
| C294 and C295 | A |
| C300a and C300b | A |
| C308a and C308b | B |
| C311 and C312 | B |
| C314a and C314b | B |
| C318 and C319 | F |
| C320 and C321 | A |
| C340a and C340b | G |
| C342 and C343 | F |
| C348 and C349 | H |
| C350 and C351 | I |
| C352 and C353 | B |

### 2. Abbreviations

| | |
|---|---|
| μw | Microwave |
| 2-MeTHF | 2-Methyltetrahydrofuran |
| AcOH | Acetic acid |
| aq | Aqueous |
| Boc₂O | Di-tert-butyl dicarbonate |
| CDI | 1,1′-Carbonyldiimidazole |
| Cu(OAc)₂ | Copper(II) acetate |
| DBU | 1,8-Diazabicyclo[5.4.0]undec-7-ene |
| DCE | 1,2-Dichloroethane |
| DCM | Dichloromethane |
| DIPE | Diisopropyl ether |
| DIPEA | N,N-Diisopropylethylamine |
| DMAP | 4-(Dimethylamino)pyridine |
| DMF | N,N-Dimethylformamide |
| DMSO | Dimethyl sulfoxide |
| Et₃N | Triethylamine |
| EtOAc | Ethyl acetate |
| EtOH | Ethanol |
| h | Hour |
| HBTU | N,N,N′,N′-Tetramethyl-O-(1H-benzotriazol-1-yl)uranium |
| | hexafluorophosphate |
| HPLC | High Liquid Performance Chromatography |
| i-PrNH₂ | Isopropylamine |
| i-PrOH | Isopropyl alcohol |
| KOAc | Potassium acetate |
| LCMS | Liquid Chromatography Mass Spectrometry |
| m-CPBA | 3-Chloroperbenzoic acid |
| Mel | Methyl iodide |
| MeOH | Methanol |
| min | Minute |
| MS | Molecular sieves |
| NaBH(OAc)₃ | Sodium triacetoxyborohydride |
| NaOAc | Sodium acetate |
| NaOMe | Sodium methylate |
| n-BuOH | n-Butyl alcohol |
| NH₄OAc | Ammonium acetate |
| NMM | 4-Methylmorpholine |
| NMR | Nuclear Magnetic Resonance |
| N-XantPhos Pd | (6-Diphenylphosphanyl-10H-phenoxazin-4-yl)- |
| G3 | diphenylphosphane; methanesulfonic acid; palladium; 2- |
| | phenylaniline |
| o/n | Overnight |
| o/WE | Over weekend |
| Pd(dppf)Cl₂ | [1,1′-Bis(diphenylphosphino)ferrocene]dichloropalladium(II) |
| Pd(dppf)Cl₂•DCM | [1,1′-Bis(diphenylphosphino)ferrocene]dichloropalladium(II) |
| | complex with dichloromethane |
| Pd(OH)₂ | Palladium hydroxide on carbon |
| Pd(PPh₃)₄ | Tetrakis(triphenylphosphine)palladium(0) |
| rt | Room temperature |
| Rt | Retention time |
| SFC | Supercritic Flash Chromatography |
| TBHP | tert-Butyl hydroperoxide |
| t-BuOK | tert-Butyl alcohol |
| TFA | Trifluoroacetic acid |
| THF | Tetrahydrofuran |
| Ti(OEt)₄ | Titanium(IV) ethoxide |
| TMEDA | N,N,N′,N′-Tetramethylethylenediamine |
| Xantphos Pd G3 | (5-Diphenylphosphanyl-9,9-dimethylxanthen-4-yl)- |
| | diphenylphosphane; methanesulfonic acid; palladium; 2- |
| | phenylaniline |
| Δ | Reflux |

### 3. Experimental Procedures

### 3.1. Synthesis of the Intermediates

### 3.1.1. Synthesis of Intermediates I7a, I7b and I7c

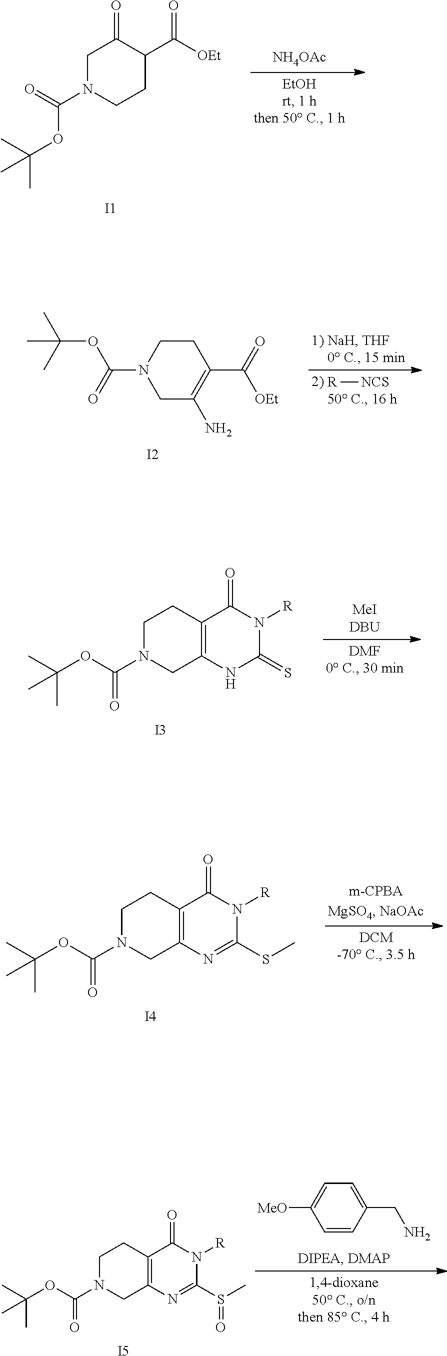

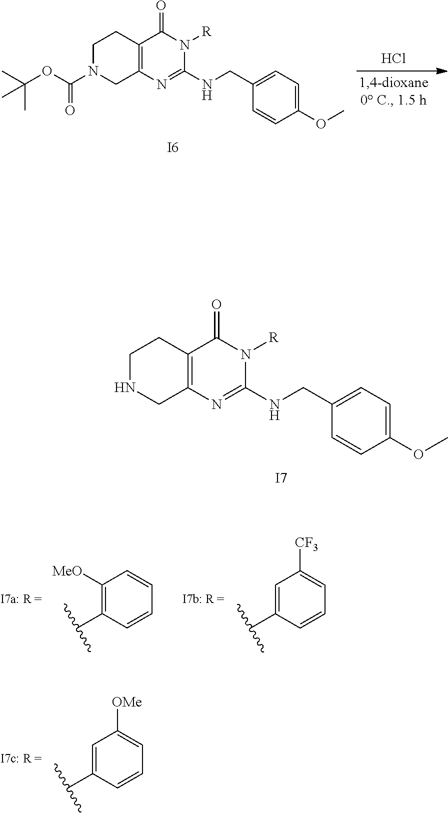

1-tert-Butyl 4-ethyl-3-oxopiperidine-1,4-dicarboxylate [71233-25-5] (Intermediate I1) (25.0 g, 92.0 mmol) was dissolved in EtOH (300 mL) under N₂ atmosphere. NH₄OAc (35.5 g, 461 mmol) was added and the reaction mixture was stirred at room temperature for 1 h. The reaction mixture was then stirred at 50° C. for 1 h, cooled to room temperature and concentrated under reduced pressure. The residue was partitioned between 2-MeTHF and a saturated aqueous solution of K₂CO₃. The layers were separated and the aqueous phase was extracted with 2-MeTHF. The combined organic extracts were dried (MgSO₄), filtered and concentrated under reduced pressure. The residue was triturated in heptane, filtered and dried under vacuum to afford 1-(tert-butyl) 4-ethyl 5-amino-3,6-dihydropyridine-1,4(2H)-dicarboxylate (Intermediate I2) (23 g, 92%) as a white solid.

To a solution of Intermediate I2 (5.79 g, 21.4 mmol) in THF (50 mL) at 0° C. under N₂ atmosphere was added NaH (60% dispersion in mineral oil, 1.91 g, 43.8 mmol) in portions. The reaction mixture was stirred at room temperature for 15 min and thereto was added 2-methoxyphenyl isothiocyanate (reagent a) (4.60 g, 27.8 mmol). The reaction mixture was stirred at 50° C. for 16 h. The reaction was quenched with a 1M aqueous solution of HCl and the mixture was extracted with 2-MeTHF. The organic layer was washed with a saturated aqueous solution of NaHCO₃, dried (MgSO₄), filtered and concentrated under reduced pressure. The residue was purified by flash column chromatography (silica, mobile phase gradient: 0 to 50% EtOAc in heptane). The residue was triturated in DIPE and filtered off to afford tert-butyl 3-(2-methoxyphenyl)-4-oxo-2-thioxo-2,3,4,5,6,8-hexahydropyrido[3,4-d]pyrimidine-7(1H)-carboxylate (Intermediate I3a) (2.4 g, 29%) as a white solid.

To a solution of Intermediate I3a (2.4 g, 6.16 mmol) in DMF (30 mL) were added DBU (1.11 mL, 7.40 mmol) and Mel (0.42 mL, 6.8 mmol) at 0° C. The reaction mixture was stirred at 0° C. for 30 min and the reaction was quenched with a saturated aqueous solution of NaHCO₃. The mixture was extracted with 2-MeTHF. The organic phase was dried (MgSO₄), filtered, and concentrated under reduced pressure. The residue was purified by flash column chromatography (silica, mobile phase gradient: 0 to 50% EtOAc in heptane) to afford tert-butyl 3-(2-methoxyphenyl)-2-(methylthio)-4-oxo-4,5,6,8-tetrahydropyrido[3,4-d]pyrimidine-7(3H)-carboxylate (Intermediate I4a) (2.2 g, 88%) as a pale yellow foam.

Intermediate I4a (500 mg, 1.24 mmol) was dissolved in DCM (20 mL). MgSO₄ (2.00 g, 16.6 mmol) and NaOAc (231 mg, 2.82 mmol) were added and the reaction mixture was cooled and stirred at −70° C. A solution of m-CPBA (990 mg, 5.7 mmol) in DCM (20 mL) was added dropwise. The reaction mixture was stirred at −70° C. for 3.5 h and then poured into a 10% aqueous solution of Na₂S₂O₃ (75 mL). The layers were separated and the organic phase was washed with a saturated aqueous solution of NaHCO₃ (50 mL), dried (Na₂SO₄), filtered and concentrated under reduced pressure to obtain tert-butyl 3-(2-methoxyphenyl)-2-(methylsulfinyl)-4-oxo-4,5,6,8-tetrahydropyrido[3,4-d]pyrimidine-7(3H)-carboxylate (Intermediate I5a) (0.49 g, 93%) as a white powder.

A mixture of Intermediate I5a (0.49 g, 1.16 mmol), 4-methoxybenzylamine (reagent b) (290 mg, 2.12 mmol), DIPEA (150 mg, 1.16 mmol) and DMAP (12.6 mg, 0.10 mmol) in 1,4-dioxane (50 mL) was stirred at 50° C. overnight and at 85° C. for 1 h.

Additional amount of 4-methoxybenzylamine (reagent b) (290 mg, 2.12 mmol) was added and the reaction mixture was stirred for another 4 h at 85° C. The reaction mixture was cooled to room temperature and concentrated under reduced pressure. The crude mixture was purified by flash column chromatography (silica, mobile phase gradient: 30 to 100% EtOAc in heptane). The product was dried at 50° C. under vacuum overnight to afford tert-butyl 2-((4-methoxybenzyl)amino)-3-(2-methoxy-phenyl)-4-oxo-4,5,6,8-tetrahydropyrido[3,4-d]pyrimidine-7(3H)-carboxylate (Intermediate I6a) (460 mg, 81%) as a clear resin.

Intermediate I6a (435 mg, 0.88 mmol) was dissolved in 1,4-dioxane (5 mL) and the solution was cooled in an ice bath. HCl (4 M in 1,4-dioxane, 5 mL, 20 mmol) was added and the reaction mixture was stirred in an ice bath for 90 min. The reaction mixture was concentrated under reduced pressure to afford 2-((4-methoxybenzyl)amino)-3-(2-methoxyphenyl)-5,6,7,8-tetrahydropyrido[3,4-d]pyrimidin-4(3H)-one dihydrochloride (Intermediate I7a) (410 mg, quant.) as a white powder.

Intermediates I7b and I7c were synthesized following the procedure described for intermediate I7a. Reagent a was 3-(trifluoromethyl)phenyl isothiocyanate and 3-methoxyphenyl isothiocyanate for the synthesis of intermediates I3b and I3c respectively.

### 3.1.2. Synthesis of Intermediates I11a, I11b and I11c

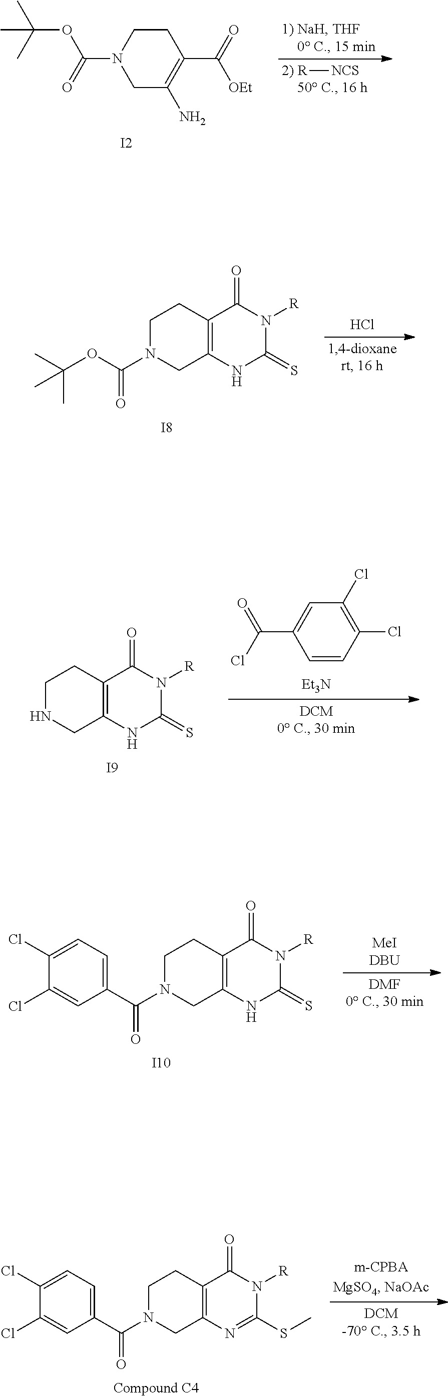

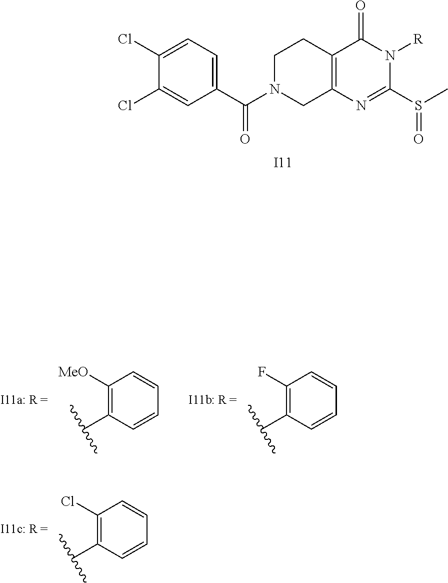

To a solution of Intermediate I2 (5.79 g, 21.4 mmol) in THF (50 mL) at 0° C. under N₂ atmosphere was added NaH (60% dispersion in mineral oil, 1.91 g, 43.8 mmol) in portions. The reaction mixture was stirred at room temperature for 15 min and thereto was added 2-methoxyphenyl isothiocyanate (reagent a) (4.60 g, 27.8 mmol). The reaction mixture was stirred at 50° C. for 16 h. The reaction was quenched with a 1M aqueous solution of HCl and the mixture was extracted with 2-MeTHF. The organic layer was washed with a saturated aqueous solution of NaHCO₃, dried (MgSO₄), filtered and concentrated under reduced pressure. The residue was purified by flash column chromatography (silica, mobile phase gradient: 0 to 50% EtOAc in heptane). The residue was triturated in DIPE and filtered off to afford tert-butyl 3-(2-methoxyphenyl)-4-oxo-2-thioxo-2,3,4,5,6,8-hexahydropyrido[3,4-d]pyrimidine-7(1H)-carboxylate (Intermediate I8a) (2.4 g, 29%) as a white solid.

Intermediate I8a (2.6 g, 6.676 mmol) was dissolved in 1,4-dioxane (100 mL) and HCl (4M in 1,4-dioxane, 40 mL, 160 mmol) was added. The reaction mixture was stirred at room temperature for 16 h. HCl (4M in 1,4-dioxane, 40 mL, 160 mmol) was added and the reaction mixture was stirred for another 4 h. The reaction mixture was concentrated under reduced pressure. The residue was triturated in DIPE, filtered and dried to afford 3-(2-methoxyphenyl)-2-thioxo-2,3,5,6,7,8-hexahydropyrido[3,4-d]pyrimidin-4(1H)-one dihydrochloride (Intermediate I9a) (2.3 g, 95%) as a yellow solid.

Intermediate I9a dihydrochloride (2.3 g, 6.35 mmol) was suspended in dry DCM (400 mL). The reaction mixture was cooled in an ice bath and Et₃N (3.53 mL, 25.4 mmol) was added followed by 3,4-dichlorobenzoyl chloride (1.44 g, 6.67 mmol). The reaction mixture was stirred at 0-5° C. for 30 min and allowed to warm to room temperature. The reaction mixture was washed with a saturated aqueous solution of NaHCO₃ (50 mL), dried (Na₂SO₄), filtered and concentrated under reduced pressure. The residue was purified by flash column chromatography (silica, mobile phase gradient: 10 to 100% EtOAc in heptane). The product was dried under vacuum to afford 7-(3,4-dichlorobenzoyl)-3-(2-methoxyphenyl)-2-thioxo-2,3,5,6,7,8-hexahydropyrido[3,4-d]pyrimidin-4(1H)-one (Intermediate I10a) (2.9 g, 99%). To a solution of Intermediate I10a (2.9 g, 6.27 mmol) in DMF (25 mL) were added DBU (1.12 mL, 7.53 mmol) and Mel (430 μL, 6.9 mmol) at 0° C. The reaction mixture was stirred at 0° C. for 30 min and the reaction was quenched with a saturated aqueous solution of NaHCO₃. The layers were separated and the aqueous phase was extracted with 2-MeTHF. The organic layer was dried (MgSO₄), filtered and concentrated under reduced pressure. The residue was purified by flash column chromatography (silica, mobile phase gradient: 0 to 50% EtOAc in heptane) to give Compound C4 (2.4 g, 80%) as a pale-yellow foam.

Compound C4 (0.5 g, 1.05 mmol) was dissolved in DCM (10 mL). MgSO₄ (1.7 g, 14 mmol) and NaOAc (196 mg, 2.39 mmol) were added and the reaction mixture was cooled and stirred at −70° C. A solution of m-CPBA (838 mg, 4.85 mmol) in DCM (15 mL) was added dropwise. The reaction mixture was stirred at −70° C. for 3.5 h and then poured into a 10% aqueous solution of Na₂S₂O₃ (75 mL). The layers were separated and the organic phase was washed with a saturated aqueous solution of NaHCO₃ (50 mL), dried (Na₂SO₄), filtered and concentrated under reduced pressure to afford 7-(3,4-dichlorobenzoyl)-3-(2-methoxyphenyl)-2-(methylsulfinyl)-5,6,7,8-tetrahydropyrido[3,4-d]pyrimidin-4(3H)-one (Intermediate I11a) (465 mg, 90%) as a white powder.

Intermediates I11b and I11c were synthesized following the procedure described for intermediate I11a. Reagent a was 2-fluorophenyl isothiocyanate and 2-chlorophenyl isothiocyanate for the synthesis of intermediates I8b and I8c respectively.

### 3.1.3. Synthesis of Intermediate I12

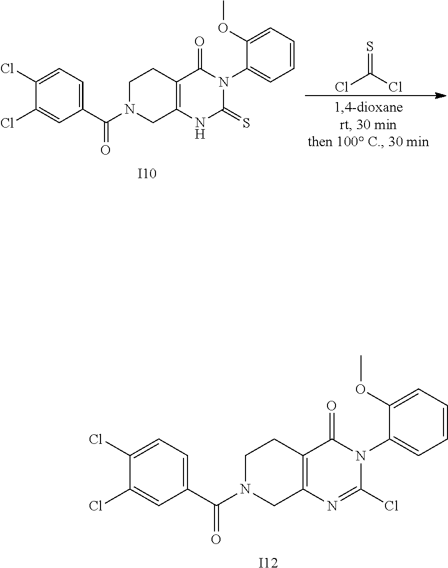

Intermediate I10 (3.00 g, 6.49 mmol) was suspended in 1,4-dioxane (45 mL) and thiophosgene (0.51 mL, 6.49 mmol) was added. The reaction mixture was stirred at room temperature for 30 min, and at 100° C. for another 30 min. The mixture was cooled to room temperature and concentrated under reduced pressure. The residue was purified by flash column chromatography (silica, mobile phase gradient: 0 to 100% EtOAc in heptane) to afford 2-chloro-7-(3,4-dichlorobenzoyl)-3-(2-methoxyphenyl)-5,6,7,8-tetrahydropyrido[3,4-d]pyrimidin-4(3H)-one (Intermediate I12) (2.6 g, 86%) as a white foam.

### 3.1.4. Synthesis of Intermediates I13, I14 and I15

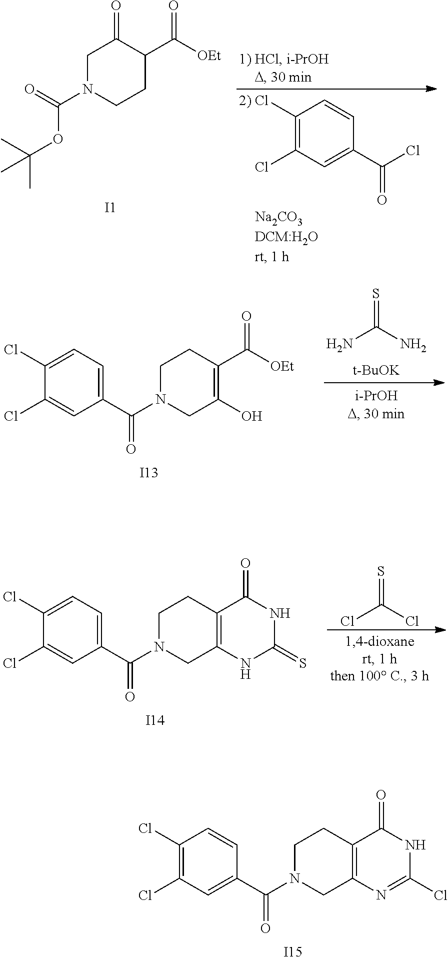

HCl (6 M in i-PrOH, 31 mL, 186 mmol) was added to a solution of Intermediate I1 (25.0 g, 92.1 mmol) in i-PrOH (250 mL) and the reaction mixture was stirred under reflux for 30 min. The reaction mixture was concentrated under reduced pressure.

The residue was triturated in DIPE, filtered off and dried. This was dissolved in a mixture of DCM (250 mL) and water (250 mL). 3,4-Dichlorobenzoyl chloride (20.9 g, 96.8 mmol) was added followed by the portion wise addition of Na₂CO₃ (19.5 g, 184 mmol) over 10 min. The reaction mixture was stirred at room temperature for 1 h. The layers were separated and the organic phase was dried (MgSO₄), filtered and concentrated under reduced pressure. The crude mixture was purified by flash column chromatography (silica, mobile phase gradient: 0 to 100% EtOAc in heptane). The residue was triturated in DIPE, filtered and dried under vacuum to afford ethyl 1-(3,4-dichlorobenzoyl)-5-hydroxy-1,2,3,6-tetrahydropyridine-4-carboxylate (Intermediate I13) (19.3 g, 61%) as a white solid.

To a suspension of Intermediate I13 (5 g, 14.5 mmol) in i-PrOH (100 mL) were added thiourea (2.21 g, 29.1 mmol) and t-BuOK (2.02 g, 18.0 mmol). The reaction mixture was stirred under reflux for 30 min. The reaction mixture was cooled to room temperature and concentrated under reduced pressure to dryness. The residue was dissolved in water (100 mL) and the mixture was acidified with HCl (1M in H₂O, 19 mL, 19 mmol) dropwise. The precipitate was filtered off and dried at 50-55° C. to afford 7-(3,4-dichlorobenzoyl)-2-thioxo-2,3,5,6,7,8-hexahydropyrido[3,4-d]pyrimidin-4(1H)-one (Intermediate I14) (4.48 g, 87%).

Thiophosgene (555 μL, 7.02 mmol) was added to a suspension of Intermediate I14 (2.00 g, 5.61 mmol) in 1,4-dioxane (50 mL). The reaction mixture was stirred at room temperature for 1 h and then stirred at 100° C. for 3 h. The reaction mixture was cooled to room temperature and concentrated under reduced pressure to dryness. The crude mixture was purified by flash column chromatography (silica, mobile phase gradient: 0 to 100% EtOAc in heptane) to afford 2-chloro-7-(3,4-dichlorobenzoyl)-5,6,7,8-tetrahydropyrido[3,4-d]pyrimidin-4(3H)-one (Intermediate I15) (1.37 g, 67%) as a light brown powder.

### 3.1.5. Synthesis of Intermediates I20 and I23

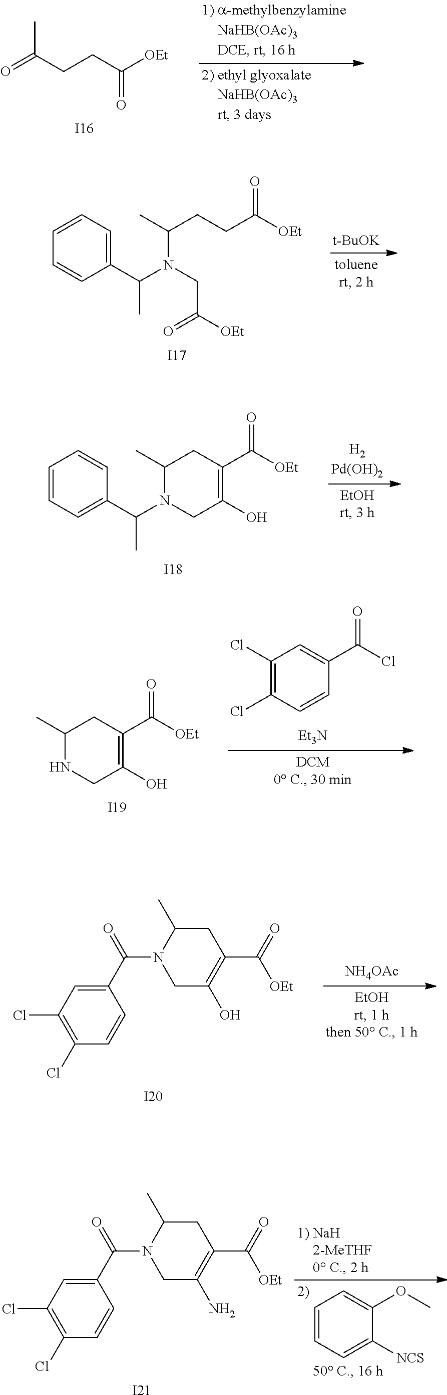

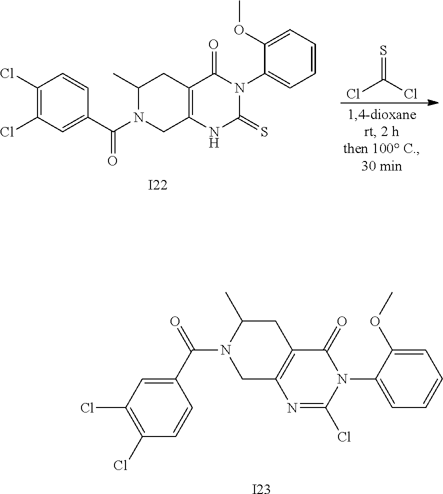

To a mixture of ethyl levulinate [539-88-8] (Intermediate I16) (10 mL, 70.3 mmol) and DL-a-methylbenzylamine (9.07 mL, 70.3 mmol) in DCE (300 mL) was added NaHB(OAc)₃ (29.8 g, 141 mmol) in portions of 5 g. The reaction mixture was stirred at room temperature for 16 h. Ethyl glyoxalate (27.8 mL, 141 mmol, 50% purity) was added followed by NaHB(OAc)₃ (14.9 g, 70.3 mmol). The reaction mixture was stirred at room temperature for 3 days. The reaction was quenched with a saturated aqueous solution of NaHCO₃ (200 mL) and solid NaHCO₃ was added until pH was 7. The layers were separated and the water layer was extracted with DCM (2×200 mL). The combined organic extracts were concentrated under reduced pressure. The crude mixture was purified by flash column chromatography (silica, mobile phase gradient: 0 to 50% EtOAc in heptane) to afford ethyl 4-((2-ethoxy-2-oxoethyl)(1-phenylethyl)amino)pentanoate (Intermediate I17) (18.1 g, 77%) as a colorless oil.

t-BuOK (11.1 g, 99.3 mmol) was added to a solution of Intermediate I17 (19.6 g, 58.4 mmol) in dry toluene (350 mL). The reaction mixture was stirred at room temperature for 2 h. The reaction was quenched with a saturated aqueous solution of NH₄Cl (150 mL). The layers were separated and the water layer was extracted with DCM (3×150 mL). The combined organic extracts were dried (MgSO₄), filtered and concentrated under reduced pressure. The crude mixture was purified by flash column chromatography (silica, mobile phase gradient: 0 to 100% EtOAc in heptane). A second purification by flash column chromatography was performed (silica, mobile phase: 1% MeOH in DCM) to afford ethyl 5-hydroxy-2-methyl-1-(1-phenylethyl)-1,2,3,6-tetrahydropyridine-4-carboxylate (Intermediate I18) (12.2 g, 71%) as an orange oil.

To a solution of Intermediate I18 (4.63 g, 16 mmol) in EtOH (120 mL) was added Pd(OH)₂ (20 wt. %, 0.23 g, 0.33 mmol). The reaction mixture was stirred at room temperature under H₂ atmosphere for 3 h. The reaction mixture was filtered over decalite and concentrated under reduced pressure to afford ethyl 5-hydroxy-2-methyl-1,2,3,6-tetrahydropyridine-4-carboxylate (Intermediate I19) (2.9 g, 91%, 93% purity) as a brown oil.

Intermediate I19 (2.9 g, 14.6 mmol, 93% purity) was dissolved in dry DCM (75 mL). The mixture was cooled in an ice bath and Et₃N (2.13 mL, 15.3 mmol) was added followed by 3,4-dichlorobenzoyl chloride (3.3 g, 15.3 mmol). The reaction mixture was stirred at 0-5° C. for 30 min and then allowed to warm to room temperature. The reaction mixture was washed with a saturated aqueous solution of NaHCO₃ (50 mL) and concentrated under reduced pressure. The crude mixture was purified by flash column chromatography (silica, mobile phase: 5% EtOAc in DCM) to afford ethyl 1-(3,4-dichlorobenzoyl)-5-hydroxy-2-methyl-1,2,3,6-tetrahydropyridine-4-carboxylate (Intermediate I20) (4.45 g, 63%, 74% purity) as a bright yellow oil.

Intermediate I20 (4.45 g, 9.19 mmol, 74% purity) was dissolved in EtOH (35 mL) under N₂ atmosphere. NH₄OAc (4.8 g, 62.1 mmol) was added and the reaction mixture was stirred at room temperature for 1 h and at 50° C. for another hour. The reaction mixture was cooled to room temperature and concentrated under reduced pressure. The residue was partitioned between 2-MeTHF (50 mL) and a saturated aqueous solution of Na₂CO₃ (50 mL). The layers were separated and the water layer was extracted with 2-MeTHF (25 mL). The combined organic layers were concentrated under reduced pressure. The crude mixture was purified by flash column chromatography (silica, mobile phase gradient: 0 to 50% EtOAc in DCM) to afford ethyl 5-amino-1-(3,4-dichlorobenzoyl)-2-methyl-1,2,3,6-tetrahydropyridine-4-carboxylate (Intermediate I21) (3.91 g, 75% over 3 steps) as a yellow powder.

To a suspension of Intermediate I21 (3.91 g, 10.9 mmol) in 2-MeTHF (30 mL) was added NaH (60% dispersion in mineral oil, 0.90 g, 22.4 mmol) at 0° C. under N₂ atmosphere. The reaction mixture was stirred at room temperature for 2 h, and thereto was added 2-methoxyphenyl isothiocyanate (2.35 g, 14.2 mmol). The reaction mixture was stirred at 50° C. for 16 h. The reaction was cooled to room temperature and neutralized with HOAc (10V % in H₂O, 20 mL). The mixture was extracted with 2-MeTHF. The organic layer was washed with a saturated aqueous solution of NaHCO₃ and concentrated under reduced pressure. The crude mixture was purified by flash column chromatography (silica, mobile phase gradient: 0 to 100% EtOAc in heptane) to afford 7-(3,4-dichlorobenzoyl)-3-(2-methoxyphenyl)-6-methyl-2-thioxo-2,3,5,6,7,8-hexahydropyrido[3,4-d]pyrimidin-4(1H)-one (Intermediate I22) (3.44 g, 66%) as an orange powder.

Intermediate I22 (1.00 g, 2.10 mmol) was dissolved in 1,4-dioxane (15 mL) and thiophosgene (0.17 mL, 2.10 mmol) was added. The reaction mixture was stirred at room temperature for 2 h and then at 100° C. for 30 min. The mixture was cooled to room temperature and concentrated under vacuum. The crude mixture was purified by flash column chromatography (silica, mobile phase gradient: 0 to 100% EtOAc in DCM) to afford 2-chloro-7-(3,4-dichlorobenzoyl)-3-(2-methoxyphenyl)-6-methyl-5,6,7,8-tetrahydropyrido[3,4-d]pyrimidin-4(3H)-one (Intermediate I23) (977 mg, 96%) as a yellow-orange foam.

### 3.1.6. Synthesis of Intermediates I26 and I27

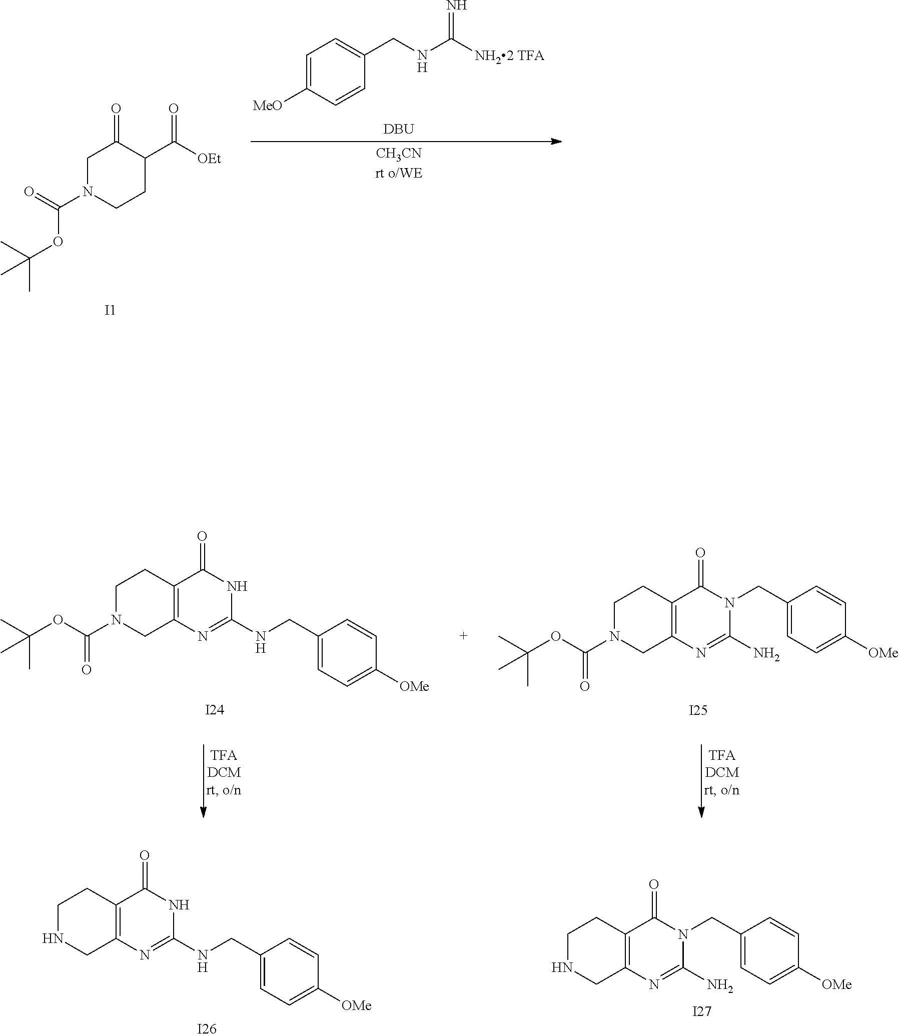

A mixture of N-[(4-methoxyphenyl)methyl]guandine.2TFA salt (10.6 g, 26.0 mmol), Intermediate I1 (7.04 g, 26.0 mmol) and DBU (15.5 mL, 104 mmol) in CH₃CN (100 mL) was stirred at room temperature over the weekend. The reaction mixture was concentrated under reduced pressure and the crude mixture was purified via preparative HPLC (Stationary phase: RP XBridge Prep C18 OBD-10 μm, 50×150 mm, Mobile phase: 0.25% NH₄HCO₃ solution in water, MeOH) to afford tert-butyl 2-((4-methoxybenzyl)amino)-4-oxo-4,5,6,8-tetrahydropyrido[3,4-d]pyrimidine-7(3H)-carboxylate (Intermediate I24) (2.18 g, 22%) and tert-butyl 2-amino-3-(4-methoxybenzyl)-4-oxo-4,5,6,8-tetrahydropyrido[3,4-d]pyrimidine-7(3H)-carboxylate (Intermediate I25) (1.93 g, 19%).

A solution of Intermediate I24 (2.14 g, 5.54 mmol) and TFA (8.5 mL, 111 mmol) in DCM (300 mL) was stirred at room temperature overnight and concentrated under reduced pressure to afford 2-{[(4-methoxyphenyl)methyl]amino}-5,6,7,8-tetrahydropyrido[3,4-d]pyrimidin-4(3H)-one.2TFA (Intermediate I26 as a 2TFA salt) (4.7 g) as an oil.

TFA (3.76 mL, 49.2 mmol) was added to a solution of Intermediate I25 (1.9 g, 4.92 mmol) in DCM (150 mL) and the reaction mixture was stirred at room temperature overnight. The reaction mixture was concentrated under reduced pressure to afford 2-amino-3-[(4-methoxyphenyl)methyl]-5,6,7,8-tetrahydropyrido[3,4-d]pyrimidin-4(3H)-one-3TFA (Intermediate I27 as a 3TFA salt) (3.75 g) as an oily resin.

### 3.1.7. Synthesis of Intermediate I28

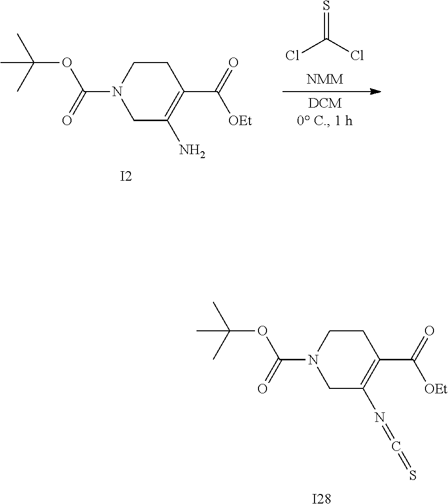

Intermediate I2 (1.00 g, 3.7 mmol) and NMM (1.02 mL, 9.25 mmol) were dissolved in DCM (10 mL). The reaction mixture was cooled in an ice bath and thiophosgene (0.37 mL, 4.6 mmol) was added dropwise at 0° C. The reaction mixture was stirred at 0° C. for 1 h. The mixture was loaded on a silica cartridge and purified by flash column chromatography (silica, mobile phase gradient: 0 to 50% EtOAc in heptane) to afford 1-(tert-butyl) 4-ethyl 5-isothiocyanato-3,6-dihydropyridine-1,4(2H)-dicarboxylate (Intermediate I28) (1.1 g, 95%) as a yellow oil.

### 3.1.8. Synthesis of Intermediate I30

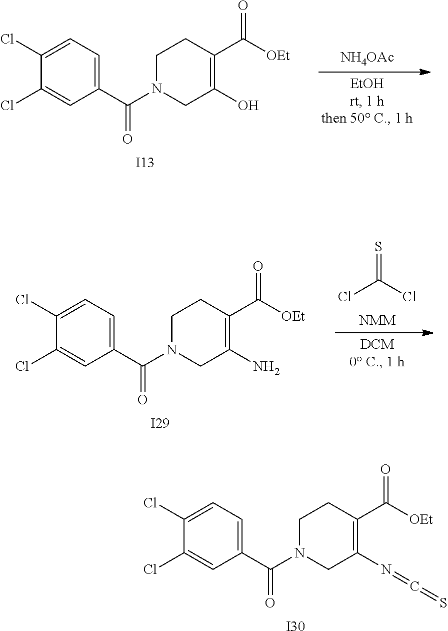

Intermediate I13 (5 g, 14.5 mmol) was dissolved in EtOH (50 mL) under N₂ atmosphere. NH₄OAc (5.60 g, 72.6 mmol) was added and the reaction mixture was stirred at room temperature for 1 h, then at 50° C. for 1 h. The reaction mixture was cooled to room temperature and concentrated under reduced pressure. The residue was partitioned between 2-MeTHF and a saturated aqueous solution of K₂CO₃. The layers were separated and the water layer was extracted with 2-MeTHF. The combined organic extracts were dried (MgSO₄), filtered and concentrated under reduced pressure to afford ethyl 5-amino-1-(3,4-dichlorobenzoyl)-1,2,3,6-tetrahydropyridine-4-carboxylate (Intermediate I29) (4.8 g, 96%) as a white solid.

Intermediate I29 (2 g, 5.83 mmol) and NMM (1.61 mL, 14.6 mmol) were dissolved in DCM (15 mL). The reaction mixture was cooled in an ice bath and thiophosgene (0.58 mL, 7.3 mmol) was added dropwise at 0° C. The reaction mixture was stirred at 0° C. for 1 h. The mixture was loaded on a silica cartridge and purified by flash column chromatography (silica, mobile phase gradient: 0 to 50% EtOAc in heptane). The residue was triturated in heptane and filtered off to afford ethyl 1-(3,4-dichlorobenzoyl)-5-isothiocyanato-1,2,3,6-tetrahydropyridine-4-carboxylate (Intermediate I30) (1.9 g, 85%) as a yellow solid.

### 3.1.9. Synthesis of Intermediates I36 and I37

The reaction sequence was carried out in the presence of ethyl and methyl ester intermediates.

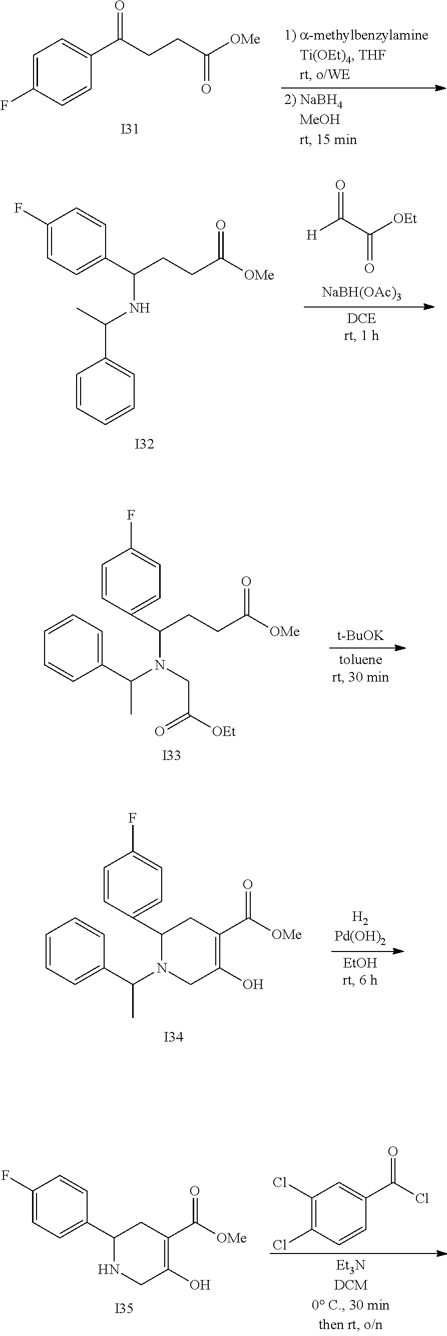

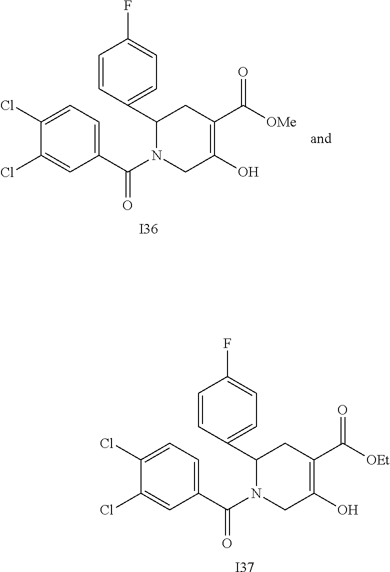 [SMILES: "I36" ; COC(=O)C1=C(O)CN(C(=O)c2ccc(Cl)c(Cl)c2)C(c2ccc(F)cc2)C1 ; "I37" ; CCOC(=O)C1=C(O)CN(C(=O)c2ccc(Cl)c(Cl)c2)C(c2ccc(F)cc2)C1]

Methyl 4-(4-fluorophenyl)-4-oxobutanoate [39560-31-1] (Intermediate I31) (1.00 g, 4.76 mmol) and DL-a-methylbenzylamine (0.8 mL, 6.18 mmol) were dissolved in dry THF (20 mL) and titanium (IV) ethoxide (2 mL, 9.56 mmol) was added. The reaction mixture was stirred at room temperature over the weekend. MeOH (9 mL) was added followed by careful addition of sodium borohydride (360 mg, 9.52 mmol). The reaction mixture was stirred for 15 min at room temperature and the reaction was quenched with a saturated aqueous solution of NaHCO₃ (3 mL). The mixture was stirred for 5 min, the precipitate was filtered off and the filtration cake was washed with EtOAc. The filtrate was evaporated under reduced pressure to dryness. The crude mixture was purified by flash column chromatography (silica, mobile phase gradient: 0 to 50% EtOAc in DCM) to afford a mixture of methyl- and ethyl-4-(4-fluorophenyl)-4-((1-phenylethyl)amino)butanoate (Intermediates I32) (1.16 g) as a yellowish oil.

To a mixture of Intermediates I32 (1.16 g) and ethyl glyoxalate (1.39 mL, 7.02 mmol, 50% purity) in DCE (8.34 mL) was added NaBH(OAc)₃ (3.72 g, 17.5 mmol) portion wise at room temperature. The suspension was stirred at room temperature for 1 h. The reaction mixture was diluted with DCM (10 mL) and a saturated aqueous solution of NaHCO₃ (20 mL) was added. The layers were separated and the aqueous layer was extracted with DCM (3×20 mL). The combined organic extracts were concentrated under reduced pressure. The crude mixture was purified by flash column chromatography (silica, mobile phase gradient: 0 to 80% EtOAc in heptane) to afford a mixture of methyl- and ethyl-4-((2-ethoxy-2-oxoethyl)(1-phenylethyl)amino)-4-(4-fluorophenyl)butanoate (Intermediates I33) (867 mg) as a yellowish oil.

t-BuOK (0.37 g, 3.30 mmol) was added to a solution of Intermediates I33 (867 mg) in dry toluene (10 mL). The reaction mixture was stirred at room temperature for 30 min and the reaction was quenched with a saturated aqueous solution of NH₄Cl (30 mL). The layers were separated and the water layer was extracted with DCM (2×20 mL). The combined organic extracts were concentrated under reduced pressure. The crude mixture was purified by flash column chromatography (silica, mobile phase gradient: 0 to 5% MeOH in DCM) to afford a mixture of methyl- and ethyl-2-(4-fluorophenyl)-5-hydroxy-1-(1-phenylethyl)-1,2,3,6-tetrahydropyridine-4-carboxylate (Intermediates I34) (526 mg) as a yellowish oil.

To a solution of Intermediates I34 (100 mg) in EtOH (10 mL) was added Pd(OH)₂ (20 wt. %, 16.2 mg, 0.023 mmol). The reaction mixture was stirred at room temperature under H₂ atmosphere for 6 h. The reaction mixture was filtered over decalite and concentrated under reduced pressure to afford a mixture of methyl- and ethyl-2-(4-fluorophenyl)-5-hydroxy-1,2,3,6-tetrahydropyridine-4-carboxylate (Intermediates I35) as a brown oil.

To a solution of Intermediates I35 in dry DCM (10 mL) was added Et₃N (0.034 mL, 0.24 mmol) followed by 3,4-dichlorobenzoyl chloride (37 mg, 0.17 mmol) at 0-5° C. The reaction mixture stirred at 0° C. for 30 min, allowed to warm to room temperature and stirred for 16 h. The reaction was quenched with a saturated aqueous solution of NaHCO₃ (3 mL). The layers were separated and the organic phase was concentrated under reduced pressure. The crude mixture was purified by flash column chromatography (silica, mobile phase gradient: 0 to 30% EtOAc in DCM) to afford a mixture of methyl 1-(3,4-dichlorobenzoyl)-2-(4-fluorophenyl)-5-hydroxy-1,2,3,6-tetrahydropyridine-4-carboxylate (Intermediate I36) and ethyl 1-(3,4-dichlorobenzoyl)-2-(4-fluorophenyl)-5-hydroxy-1,2,3,6-tetrahydropyridine-4-carboxylate (intermediate 137) (44 mg) as a bright yellow oil.

### 3.1.10. Synthesis of Intermediate I42

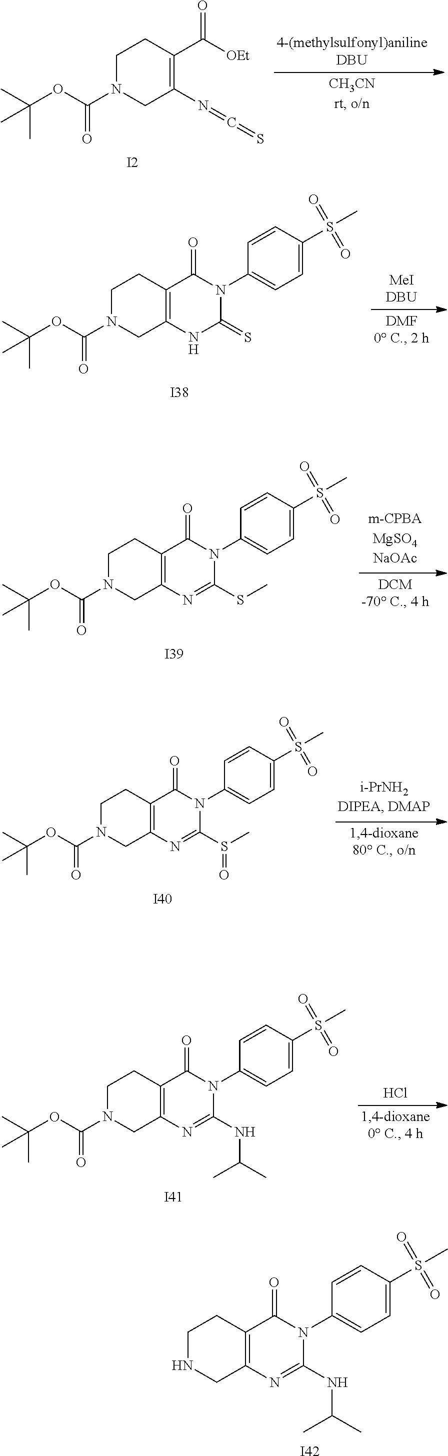

A mixture of Intermediate I2 (513 mg, 1.64 mmol), 4-(methylsulfonyl)aniline (366 mg, 2.14 mmol) and DBU (0.53 mL, 3.55 mmol) in dry CH₃CN (4 mL) was stirred at room temperature overnight. The mixture was loaded on a silica cartridge and purified by flash column chromatography (silica, mobile phase gradient: 0 to 100% EtOAc in heptane) to afford tert-butyl 3-(4-(methylsulfonyl)phenyl)-4-oxo-2-thioxo-2,3,4,5,6,8-hexahydropyrido[3,4-d]pyrimidine-7(1H)-carboxylate (Intermediate I38) (431 mg) as a yellow oil.

To a solution of Intermediate I38 (1.84 g, 1.98 mmol, 47% purity) in dry DMF (22 mL) were added DBU (0.9 mL, 6.02 mmol) and Mel (0.35 mL, 5.62 mmol) at 0° C. The reaction mixture was stirred at 0° C. for 2 h and the reaction was quenched with a saturated aqueous solution of NaHCO₃. The mixture was extracted with 2-MeTHF (2×20 mL). The combined organic extracts were dried (MgSO₄), filtered and concentrated under reduced pressure. The residue was purified by flash column chromatography (silica, mobile phase gradient: 0 to 50% EtOAc in heptane) to afford tert-butyl 3-(4-(methylsulfonyl)phenyl)-2-(methylthio)-4-oxo-4,5,6,8-tetrahydropyrido[3,4-d]pyrimidine-7(3H)-carboxylate (Intermediate I39) (1.5 g, 60% purity) as a pale yellow solid.

Intermediate I39 (2.17 g, 3.51 mmol, 73% purity) was dissolved in dry DCM (40 mL). MgSO₄ (6.68 g, 55.5 mmol) and NaOAc (800 mg, 9.75 mmol) were added and the mixture was cooled to −70° C. A solution of m-CPBA (3.50 g, 20.3 mmol) in dry DCM (50 mL) was added dropwise. The reaction mixture was stirred at −70° C. for 4 h and poured into a 10% aqueous solution of Na₂S₂O₃ (˜100 mL). The layers were separated and the aqueous phase was extracted with DCM (2×100 mL). The combined organic extracts were washed with a saturated aqueous solution of NaHCO₃ (50 mL), dried (MgSO₄), filtered and concentrated under reduced pressure to afford tert-butyl 2-(methylsulfinyl)-3-(4-(methylsulfonyl)phenyl)-4-oxo-4,5,6,8-tetrahydropyrido[3,4-d]pyrimidine-7(3H)-carboxylate (Intermediate I40) (2.45 g, 87%, 58% purity).

A mixture of Intermediate I40 (2.46 g, 3.57 mmol, 68% purity), isopropylamine (0.7 mL, 8.17 mmol), DIPEA (1 mL, 5.84 mmol) and DMAP (90 mg, 737 μmol) in dry 1,4-dioxane (70 mL) was stirred at 80° C. overnight. The reaction mixture was concentrated under reduced pressure and the residue was purified by flash column chromatography (silica, mobile phase gradient: 30 to 100% EtOAc in heptane) to afford tert-butyl 2-(isopropylamino)-3-(4-(methylsulfonyl)phenyl)-4-oxo-4,5,6,8-tetrahydropyrido[3,4-d]pyrimidine-7(3H)-carboxylate (Intermediate I41) (0.9 g, 55%).

Intermediate I41 (0.90 g, 1.95 mmol) was dissolved in 1,4-dioxane (11 mL). The solution was cooled in an ice bath and HCl (4M in 1,4-dioxane, 11 mL, 44.0 mmol) was added. The reaction mixture was stirred in the ice bath for 4.5 h. The reaction mixture was concentrated under reduced pressure to afford 3-[4-(methanesulfonyl)phenyl]-2-[(propan-2-yl)amino]-5,6,7,8-tetrahydropyrido[3,4-d]pyrimidin-4(3H)-one hydrochloride (Intermediate I42 as a HCl salt) (816 mg).

### 3.1.11. Synthesis of Intermediates I43-(R), I43-(S) and I45-(R)

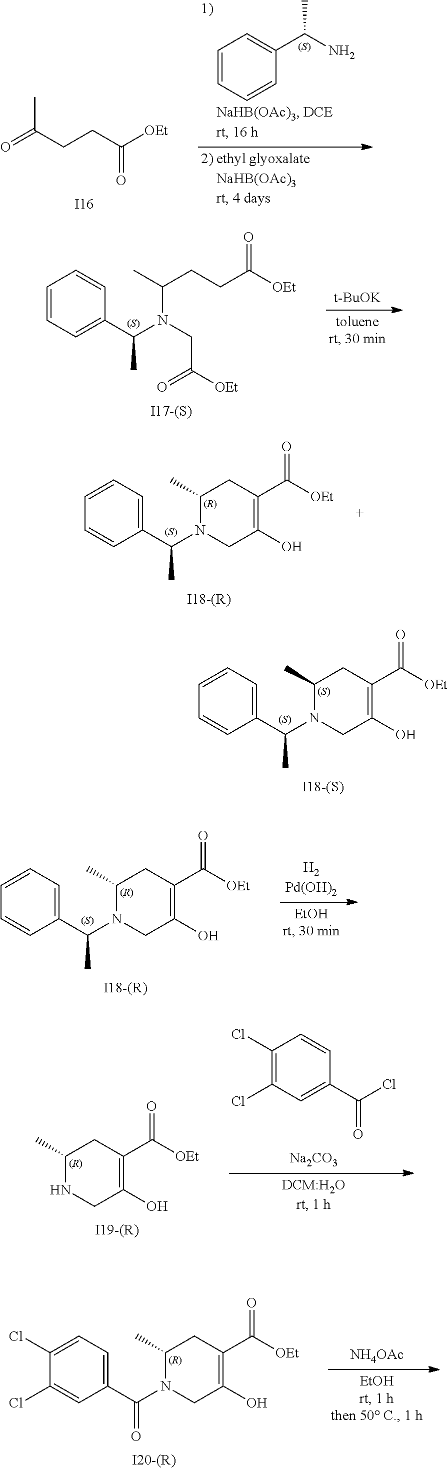

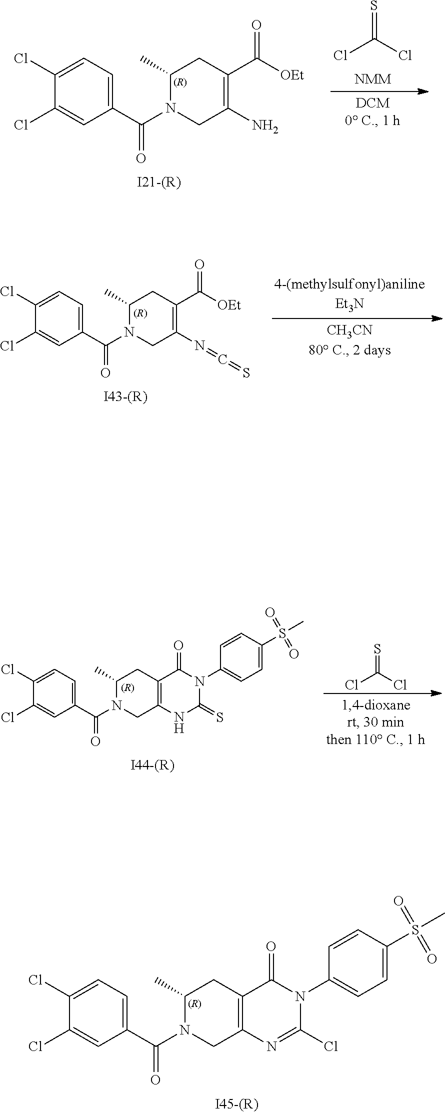

To a mixture of ethyl levulinate [539-88-8] (Intermediate I16) (20 mL, 141 mmol) and (S)-(−)-1-phenylethylamine (18.1 mL, 141 mmol) in DCE (341 mL) was added NaHB(OAc)₃ (44.7 g, 211 mmol). The reaction mixture was stirred at room temperature for 16 h. Ethyl glyoxalate (55.7 mL, 281 mmol) was added followed by NaHB(OAc)₃ (44.7 g, 211 mmol). The reaction mixture was stirred at room temperature for 4 days. The reaction was quenched with a saturated aqueous solution of NaHCO₃ (350 mL) and NaHCO₃ was added until pH was 7. The layers were separated and the aqueous phase was extracted with DCM (2×300 mL). The combined organic extracts were concentrated under reduced pressure to afford ethyl 4-((2-ethoxy-2-oxoethyl)((S)-1-phenylethyl)amino)pentanoate (Intermediate I17-(S)). The crude product was used in the next step.

t-BuOK (39.4 g, 351 mmol) was added to a solution of Intermediate I17-(S) in dry toluene (529 mL). The reaction mixture was stirred at room temperature for 30 min. The reaction was quenched with a saturated aqueous solution of NH₄Cl (500 mL) and the mixture was stirred for 15 min. The layers were separated and the aqueous phase was extracted with DCM (400 mL). The combined organic extracts were concentrated under reduced pressure. The crude mixture was purified by flash column chromatography (silica, mobile phase: 2.5% EtOAc in heptane) to afford ethyl (*S)-5-hydroxy-2-methyl-1-((S)-1-phenylethyl)-1,2,3,6-tetrahydropyridine-4-carboxylate (Intermediate I18-(S)) (10.9 g, 27% over 2 steps) and ethyl (R)-5-hydroxy-2-methyl-1-((S)-1-phenylethyl)-1,2,3,6-tetrahydropyridine-4-carboxylate (Intermediate I18-(R)) (5.13 g, 12% over 2 steps).

To a solution of Intermediate I18-(R) (5.13 g, 17.7 mmol) in EtOH (133 mL) was added Pd(OH)₂ (20 wt. %, 1.25 g, 1.77 mmol). The mixture was degassed and filled with H₂. The reaction mixture was stirred at room temperature for 30 min. The mixture was filtered over decalite and the filtrate was concentrated under reduced pressure to afford (R)-5-hydroxy-2-methyl-1,2,3,6-tetrahydropyridine-4-carboxylate (Intermediate I19-(R)). The crude product was used in the next step.

Intermediate I19-(R) was dissolved in DCM (65 mL) and water (65 mL). 3,4-Dichlorobenzoyl chloride (reagent a) (4.02 g, 18.6 mmol) was added followed by Na₂CO₃ (9.39 g, 88.6 mmol). The reaction mixture was stirred at room temperature for 1 h. The layers were separated and the organic phase was concentrated under reduced pressure. The crude mixture was purified by flash column chromatography (silica, mobile phase gradient: 0 to 50% DCM in EtOAc) to afford ethyl (R)-1-(3,4-dichlorobenzoyl)-5-hydroxy-2-methyl-1,2,3,6-tetrahydropyridine-4-carboxylate (Intermediate I20-(R)) (5.68 g, 78% over 2 steps) as a bright yellow oil.

Intermediate I20-(R) (5.68 g, 15.9 mmol) was dissolved in EtOH (43 mL). NH₄OAc (6.11 g, 79.3 mmol) was added and the reaction mixture was stirred at room temperature for 1 h and at 50° C. for another hour. The reaction mixture was cooled to room temperature and concentrated under reduced pressure. The residue was partitioned between 2-MeTHF (50 mL) and a saturated aqueous solution of Na₂CO₃ (50 mL). The layers were separated and the aqueous phase was extracted with 2-MeTHF (50 mL). The combined organic extracts were concentrated under reduced pressure. The crude mixture was purified by flash column chromatography (silica, mobile phase gradient: 0 to 30% EtOAc in DCM) to afford ethyl (R)-5-amino-1-(3,4-dichlorobenzoyl)-2-methyl-1,2,3,6-tetrahydropyridine-4-carboxylate (Intermediate I21-(R)) (5.42 g, 96%) as a white foam.

A mixture of Intermediate I21-(R) (5.42 g, 15.2 mmol) and NMM (4.2 mL, 37.9 mmol) in dry DCM (41 mL) was cooled in an ice bath. Thiophosgene (1.50 mL, 19.0 mmol) was added dropwise. The reaction mixture was stirred at 0° C. for 1 h. The reaction mixture was loaded on a silica cartridge and the mixture was purified by flash column chromatography (silica, mobile phase gradient: 0 to 50% EtOAc in heptane) to afford ethyl (R)-1-(3,4-dichlorobenzoyl)-5-isothiocyanato-2-methyl-1,2,3,6-tetrahydro-pyridine-4-carboxylate (Intermediate I43-(R)) (5.86 g, 95%) as a sticky yellow oil/foam.

A mixture of Intermediate I43-(R) (3.00 g, 7.51 mmol), 4-(methylsulfonyl)aniline (1.72 g, 9.77 mmol) and Et₃N (1.57 mL, 11.3 mmol) in dry CH₃CN (40 mL) was stirred at 80° C. for 2 days. Volatiles were evaporated under reduced pressure. The crude mixture was purified by flash column chromatography (silica, mobile phase gradient: 0 to 100% EtOAc in heptane). The residue was triturated in MeOH, filtered and washed with DIPE to afford (R)-7-(3,4-dichlorobenzoyl)-6-methyl-3-(4-(methylsulfonyl)phenyl)-2-thioxo-2,3,5,6,7,8-hexahydropyrido[3,4-d]pyrimidin-4(1H)-one (Intermediate I44-(R)) (1.47 g, 37%).

A microwave tube was charged with Intermediate I44-(R) (1.47 g, 2.81 mmol) in 1,4-dioxane (15.6 mL) and sealed under N₂ atmosphere. Thiophosgene (222 μL, 2.81 mmol) was added and the reaction mixture was stirred at room temperature for 30 min and at 110° C. for 1 h. The mixture was loaded on a silica cartridge and purified by flash column chromatography (silica, mobile phase gradient: 0 to 100% EtOAc in heptane) to afford (R)-2-chloro-7-(3,4-dichlorobenzoyl)-6-methyl-3-(4-(methyl-sulfonyl)phenyl)-5,6,7,8-tetrahydropyrido[3,4-d]pyrimidin-4(3H)-one (Intermediate I45-(R)) (1.2 g, 72%, 89% purity) as a yellow foam.

Intermediate I45-(S) was synthesized following this procedure starting from intermediate I18-(S).

### 3.1.12. Synthesis of Intermediate I56

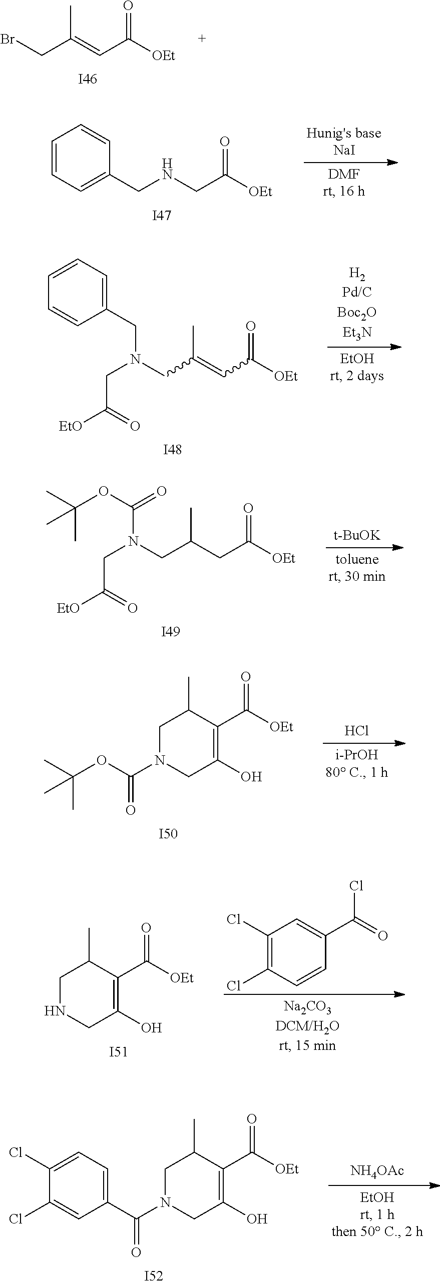

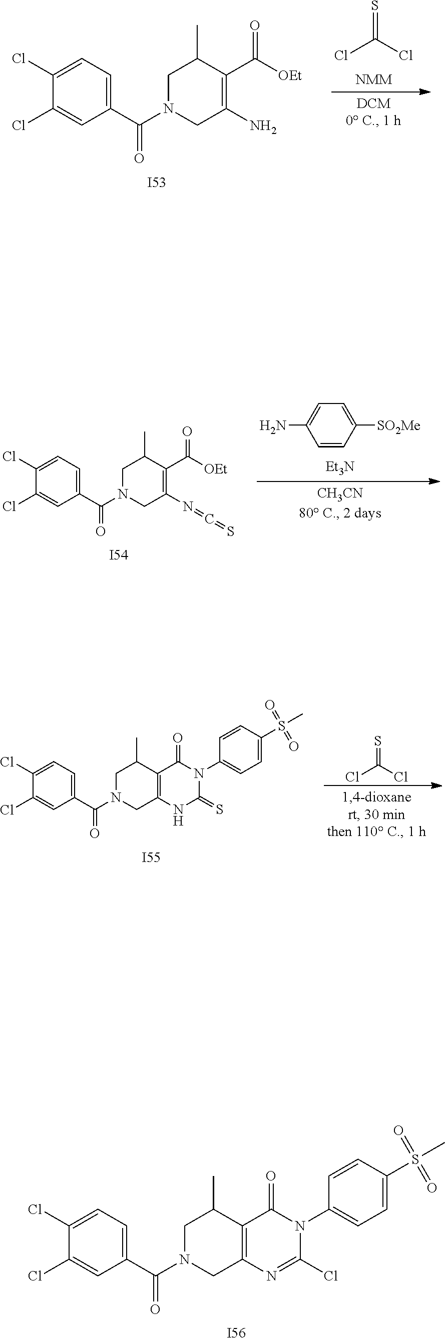

A mixture of ethyl 4-bromo-3-methylbut-2-enoate [26918-14-9] (Intermediate I46) (5 mL, 31.4 mmol), N-benzylglycine ethyl ester [6436-90-4] (Intermediate I47) (7.10 mL, 37.7 mmol), Hunig's base (10.8 mL, 62.8 mmol) and NaI (7.06 g, 47.1 mmol) in dry DMF (243 mL) was stirred at room temperature for 16 h. The reaction was quenched with a saturated aqueous solution of NaHCO₃ (100 mL). The layers were separated and the aqueous phase was extracted with EtOAc (2×100 mL). The combined organic extracts were dried (Na₂SO₄), filtered and concentrated under reduced pressure. The crude mixture was purified by flash column chromatography (silica, mobile phase gradient: 0 to 100% EtOAc in heptane) to afford ethyl 4-(benzyl(2-ethoxy-2-oxoethyl)amino)-3-methylbut-2-enoate (Intermediate I48) (8.84 g, 88%) as a yellow oil.

To a mixture of Intermediate I48 (3.06 g, 9.58 mmol), Et₃N (1.34 mL, 9.58 mmol) and boc anhydride (2.09 g, 9.58 mmol) in EtOH (42 mL) was added Pd/C (10 wt. %, 1.34 g, 1.26 mmol). The reaction mixture was stirred at room temperature for 2 days under H₂ atmosphere. The reaction mixture was filtered over decalite and the filtrate was concentrated under reduced pressure. The residue was partitioned between 2-MeTHF and water. The layers were separated and the organic phase was dried (MgSO₄), filtered and concentrated under reduced pressure. The crude mixture was purified by flash column chromatography (silica, mobile phase gradient: 0 to 60% EtOAc in heptane) to afford ethyl 4-((tert-butoxycarbonyl)(2-ethoxy-2-oxoethyl)amino)-3-methylbutanoate (Intermediate I49) (2.17 g, 61%, 89% purity) as a yellow oil.

t-BuOK (1.79 g, 16.0 mmol) was added to a solution of Intermediate I49 (2.12 g, 6.40 mmol) in dry toluene (24 mL). The reaction mixture was stirred at room temperature for 30 min. The reaction was quenched with a saturated aqueous solution of NH₄Cl (50 mL) and the mixture was stirred for 15 min. The layers were separated and the aqueous phase was extracted with EtOAc (40 mL). The combined organic extracts were concentrated under reduced pressure. The crude mixture was purified by flash column chromatography (silica, mobile phase gradient: 0 to 100% EtOAc in heptane) to afford 1-(tert-butyl) 4-ethyl 5-hydroxy-3-methyl-3,6-dihydropyridine-1,4(2H)-dicarboxylate (Intermediate I50) (263 mg, 14%) as a yellow oil.

Intermediate I50 (260 mg, 0.91 mmol) was dissolved in HCl (6M in i-PrOH, 0.74 mL, 4.45 mmol) and the solution was stirred at 80° C. for 1 h. The solvents were evaporated under reduced pressure and the residue was co-evaporated with CH₃CN to afford ethyl 5-hydroxy-3-methyl-1,2,3,6-tetrahydropyridine-4-carboxylate (Intermediate I51). The crude product was used in the next step without further purification.

Intermediate I51 was dissolved in DCM (2 mL). Water (2 mL), 3,4-dichlorobenzoyl chloride (200 mg, 0.96 mmol) and Na₂CO₃ (483 mg, 4.56 mmol) were added. The reaction mixture was stirred at room temperature for 15 min and diluted with DCM (2 mL) and water (2 mL). The layers were separated and the organic phase was concentrated under reduced pressure. The crude mixture was purified by flash column chromatography (silica, mobile phase gradient: 0 to 50% EtOAc in DCM) to afford ethyl 1-(3,4-dichlorobenzoyl)-5-hydroxy-3-methyl-1,2,3,6-tetrahydropyridine-4-carboxylate (Intermediate I52) (300 mg, 92% over 2 steps) as a clear yellow oil.

Intermediate I52 (300 mg, 837 μmol) was dissolved in EtOH (14.2 mL) and NH₄OAc (323 mg, 4.19 mmol) was added. The reaction mixture was stirred at room temperature for 1 h and at 50° C. for 2 h. Solvent was evaporated under reduced pressure and the residue was partitioned between 2-MeTHF and a saturated aqueous solution of Na₂CO₃. The layers were separated and the organic layer was concentrated under reduced pressure. The crude mixture was purified by flash column chromatography (silica, mobile phase gradient: 0 to 50% EtOAc in DCM) to afford ethyl 5-amino-1-(3,4-dichlorobenzoyl)-3-methyl-1,2,3,6-tetrahydropyridine-4-carboxylate (Intermediate I53) (263 mg, 85%) as a white foam.

A mixture of Intermediate I53 (263 mg, 714 μmol) and NMM (197 μL, 1.79 mmol) in dry DCM (1.93 mL) was cooled in an ice bath. Thiophosgene (70.5 μL, 0.89 mmol) was added dropwise and the reaction mixture was stirred at 0° C. for 1 h. The reaction mixture was loaded on a silica cartridge and the mixture was purified by flash column chromatography (silica, mobile phase gradient: 0 to 50% EtOAc in heptane) to afford ethyl 1-(3,4-dichlorobenzoyl)-5-isothiocyanato-3-methyl-1,2,3,6-tetrahydropyridine-4-carboxylate (Intermediate I54) (237 mg, 81%) as a yellow oil.

In a sealed tube, a mixture of Intermediate I54 (236 mg, 579 μmol), 4-(methyl-sulfonyl)aniline (204 mg, 1.16 mmol) and Et₃N (0.24 mL, 1.74 mmol) in dry CH₃CN (3.1 mL) was stirred at 80° C. for 2 days. The reaction mixture was cooled to room temperature and concentrated under reduced pressure. The crude mixture was purified by flash column chromatography (silica, mobile phase gradient: 0 to 100% EtOAc in DCM) to afford 7-(3,4-dichlorobenzoyl)-5-methyl-3-(4-(methylsulfonyl)-phenyl)-2-thioxo-2,3,5,6,7,8-hexahydropyrido[3,4-d]pyrimidin-4(1H)-one (Intermediate I55) (122 mg, 38%) as a yellow solid.

In a sealed tube, to a solution of Intermediate I55 (120 mg, 229 μmol) in dry 1,4-dioxane (1.9 mL) was added thiophosgene (19.9 μL, 252 μmol). The reaction mixture was stirred at room temperature for 30 min and at 110° C. for 1 h. The reaction mixture was cooled to room temperature and loaded on a silica cartridge. The mixture was purified by flash column chromatography (silica, mobile phase gradient: 0 to 100% EtOAc in heptane) to afford 2-chloro-7-(3,4-dichlorobenzoyl)-5-methyl-3-(4-(methylsulfonyl)phenyl)-5,6,7,8-tetrahydropyrido[3,4-d]pyrimidin-4(3H)-one (Intermediate I56) (108 mg, 73%, 82% purity) as a yellow foam.

### 3.1.13. Synthesis of Intermediate I64

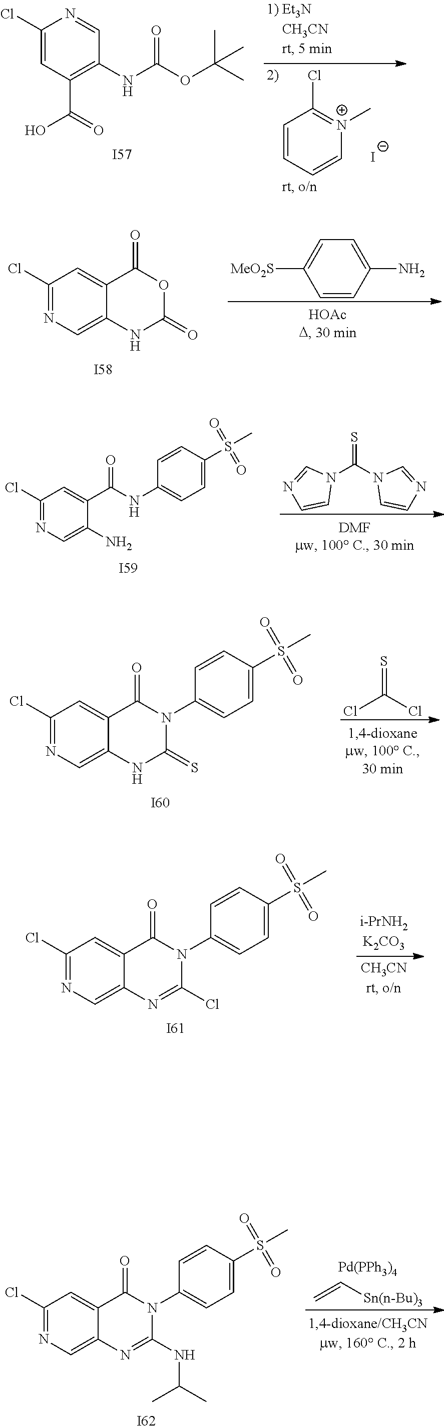

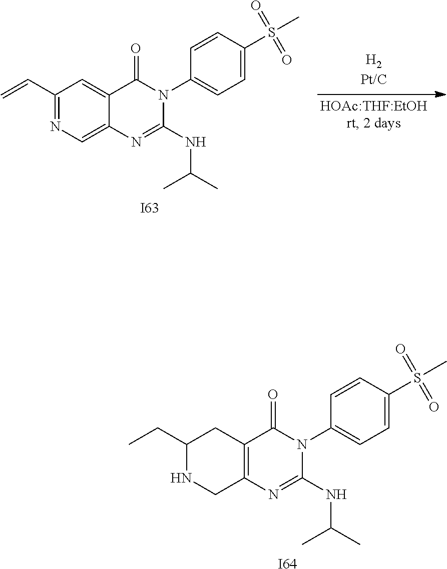

5-((tert-Butoxycarbonyl)amino)-2-chloroisonicotinic acid [171178-46-4] (Intermediate I57) (9.59 g, 35.2 mmol) was suspended in CH₃CN (150 mL). Et₃N (9.77 mL, 70.3 mmol) was added and the reaction mixture was stirred at room temperature for 5 min. 2-Chloro-1-methylpyridinium iodide (10.8 g, 42.1 mmol) was added and the reaction mixture was stirred at room temperature overnight. The reaction mixture was filtered and the filtrate was concentrated under reduced pressure. The crude mixture was purified by flash column chromatography (silica, mobile phase gradient: 30 to 100% EtOAc in heptane) to afford 6-chloro-2H-pyrido[3,4-d][1,3]oxazine-2,4(1H)-dione (Intermediate I58) (5.3 g, 76%) as a yellow powder.

A mixture of Intermediate I58 (4.57 g, 26.7 mmol) in acetic acid (150 mL) was stirred under reflux for 30 min. The reaction mixture was concentrated under reduced pressure. The residue was dissolved in THF (300 mL) and washed with an aqueous solution of NaHCO₃. The aqueous phase was extracted with THF (100 mL) and the combined organic extracts were concentrated under reduced pressure. The residue was dried under vacuum at 50° C. to afford 5-amino-2-chloro-N-(4-(methylsulfonyl)-phenyl)isonicotinamide (Intermediate I59) (quant.) as a yellow powder.

A mixture of Intermediate I59 (1.04 g, 3.18 mmol) and 1,1′-thiocarbonyldiimidazole (0.88 g, 4.92 mmol) in DMF (10 mL) was heated at 100° C. for 30 min in a Biotage microwave. The mixture was loaded on a silica cartridge and purified by flash column chromatography (silica, mobile phase gradient: 30 to 100% EtOAc in heptane) to afford 6-chloro-3-(4-(methylsulfonyl)phenyl)-2-thioxo-2,3-dihydropyrido[3,4-d]pyrimidin-4(1H)-one (Intermediate I60) (630 mg, 54%) as a yellow powder.

A mixture of Intermediate I60 (1.00 g, 2.72 mmol) and thiophosgene (0.81 g, 6.80 mmol) in 1,4-dioxane (10 mL) was heated at 100° C. for 30 min in a microwave. Additional amount of thiophosgene (400 mg, 3.37 mmol) was added and the reaction mixture was heated at 100° C. for another 30 min in a microwave. The reaction mixture was filtered. The filtrate was loaded on a silica cartridge and purified by flash column chromatography (silica, mobile phase gradient: 15 to 100% EtOAc in heptane) to afford 2,6-dichloro-3-(4-(methylsulfonyl)phenyl)pyrido[3,4-d]pyrimidin-4(3H)-one (Intermediate I61) (500 mg, 50%) as a light yellow powder.

Intermediate I61 (500 mg, 1.35 mmol) was dissolved in CH₃CN (150 mL). K₂CO₃ (0.37 g, 2.70 mmol) was added followed by isopropylamine (798 mg, 13.5 mmol). The reaction mixture was stirred at room temperature overnight. The reaction mixture was filtered. The filtrate was slowly diluted with water (350 mL) and crystallization occurred. The light yellow crystals were filtered off and dried under vacuum at 50° C. to afford 6-chloro-2-(isopropylamino)-3-(4-(methylsulfonyl)phenyl)pyrido[3,4-d]pyrimidin-4(3H)-one (Intermediate I62) (261 mg, 49%).

N₂ was bubbled through a mixture of Intermediate I62 (241 mg, 0.65 mmol) and tributyl(vinyl)tin (426 mg, 1.30 mmol) in 1,4-dioxane (10 mL) and CH₃CN (3 mL) for 10 min. Pd(PPh₃)₄ (188 mg, 163 μmol) was added and the reaction mixture was heated at 160° C. for 2 h in a Biotage microwave. The mixture was loaded on a silica cartridge and purified by flash column chromatography (silica, mobile phase gradient: 30 to 100% EtOAc in heptane) to afford 2-(isopropylamino)-3-(4-(methylsulfonyl)phenyl)-6-vinylpyrido[3,4-d]pyrimidin-4(3H)-one (Intermediate I63) (161 mg, 68%) as a white powder.

Intermediate I63 (160 mg, 416 μmol) was dissolved in a warm mixture of acetic acid (238 μL, 4.16 mmol), EtOH (25 mL) and THF (25 mL). The reaction mixture was cooled to room temperature and Pt/C (30 mg) was added under N₂ atmosphere. The reaction mixture was stirred at room temperature under H₂ atmosphere overnight. Additional amount of Pt/C was added and the reaction mixture was stirred under H₂ atmosphere for 2 days. The reaction mixture was filtered and the filtrate was concentrated under reduced pressure to afford 6-ethyl-2-(isopropylamino)-3-(4-(methylsulfonyl)phenyl)-5,6,7,8-tetrahydropyrido[3,4-d]pyrimidin-4(3H)-one (159 mg, 98%) (Intermediate I64) as a crude oil.

### 3.1.14. Synthesis of Intermediate I67

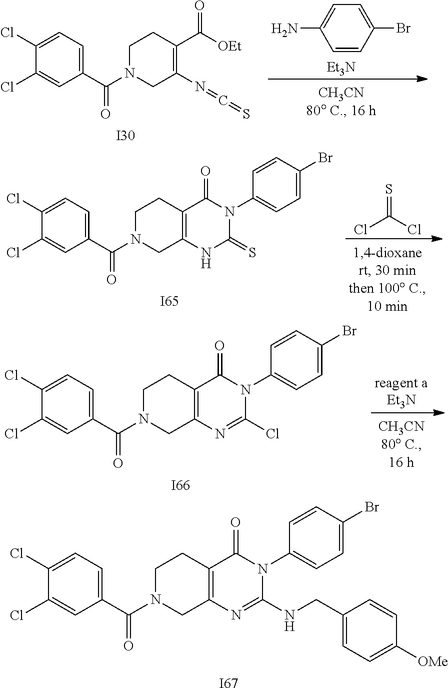

In a sealed tube, a mixture of Intermediate I30 (1.00 g, 2.60 mmol)), 4-bromoaniline (605 mg, 3.37 mmol), Et₃N (0.54 mL, 3.89 mmol) and dry CH₃CN (15 mL) was stirred at 80° C. for 16 h. The reaction mixture was cooled to room temperature. The precipitate was filtered off, washed with DIPE and dried under vacuum to afford 3-(4-bromophenyl)-7-(3,4-dichlorobenzoyl)-2-thioxo-2,3,5,6,7,8-hexahydropyrido[3,4-d]pyrimidin-4(1H)-one (Intermediate I65) (1.2 g, 90%) as a white solid.

To a solution of Intermediate I65 (805 mg, 1.575 mmol) in dry 1,4-dioxane (15 mL) in a sealed tube was added thiophosgene (137 μL, 1.73 mmol). The reaction mixture was stirred at room temperature for 30 min and at 100° C. for 10 min. The reaction mixture was cooled to room temperature and concentrated under reduced pressure. The crude mixture was purified by flash column chromatography (silica, mobile phase gradient: 0 to 50% EtOAc in heptane) to afford 3-(4-bromophenyl)-2-chloro-7-(3,4-dichlorobenzoyl)-5,6,7,8-tetrahydropyrido[3,4-d]pyrimidin-4(3H)-one (Intermediate I66) (765 mg, 95%) as a white powder.

In a sealed tube, a mixture of Intermediate I66 (765 mg, 1.49 mmol), 4-methoxybenzylamine (reagent a) (234 μL, 1.79 mmol) and Et₃N (0.41 mL, 2.98 mmol) in dry CH₃CN (15 mL) was stirred at 80° C. for 16 h. The reaction mixture was cooled to room temperature and concentrated under reduced pressure. The crude mixture was purified by flash column chromatography (silica, mobile phase gradient: 0 to 100% EtOAc in heptane). The residue was triturated in DIPE, filtered and dried under vacuum to afford 3-(4-bromophenyl)-7-(3,4-dichlorobenzoyl)-2-((4-methoxybenzyl)amino)-5,6,7,8-tetrahydropyrido[3,4-d]pyrimidin-4(3H)-one (Intermediate I67) (719 mg, 79%) as a white powder.

### 3.1.15. Synthesis of Intermediate I69

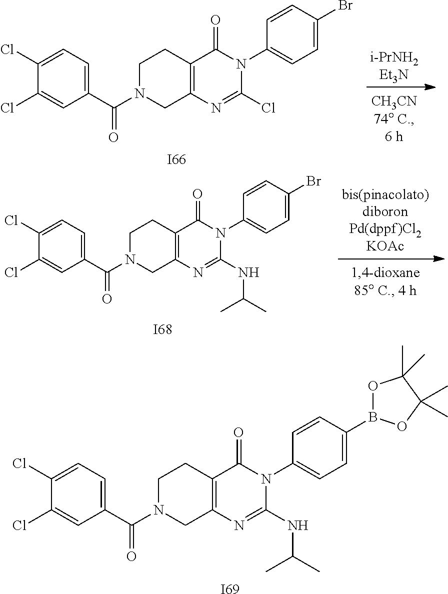 [SMILES: "I68" ; "I66" ; "I69" ; O=C(c1ccc(Cl)c(Cl)c1)N1CCc2c(nc(Cl)n(-c3ccc(Br)cc3)c2=O)C1 ; CC(C)Nc1nc2c(c(=O)n1-c1ccc(Br)cc1)CCN(C(=O)c1ccc(Cl)c(Cl)c1)C2 ; CC(C)Nc1nc2c(c(=O)n1-c1ccc(B3OC(C)(C)C(C)(C)O3)cc1)CCN(C(=O)c1ccc(Cl)c(Cl)c1)C2]

A mixture of Intermediate I66 (9.71 g, 18.9 mmol), isopropylamine (1.97 mL, 22.7 mmol), Et₃N (5.24 mL, 37.8 mmol) and CH₃CN (100 mL) was stirred at 74° C. for 6 h. The reaction mixture was cooled to 15° C. and the precipitate was filtered off and dried at 50-55° C. overnight. The residue was dissolved in DCM (25 mL) and the organic phase was washed with a saturated aqueous solution of NaHCO₃ (15 mL), dried (MgSO₄), filtered and concentrated under reduced pressure to dryness to afford a first crop of 3-(4-bromophenyl)-7-(3,4-dichlorobenzoyl)-2-(isopropylamino)-5,6,7,8-tetrahydropyrido[3,4-d]pyrimidin-4(3H)-one (Intermediate I68) (4.72 g, 47%) as a white powder. The filtrate was concentrated under reduced pressure to dryness and the residue was purified by flash column chromatography (silica, mobile phase gradient: 0 to 100% EtOAc in heptane) to afford a second crop of Intermediate I68 (4.4 g, 43%).

In a sealed tube, a mixture of Intermediate I68 (300 mg, 55.9 μmol), bis(pinacolato)diboron (284 mg, 1.12 mmol), KOAc (115 mg, 1.18 mmol) and Pd(dppf)Cl₂.DCM (46.2 mg, 55.9 μmol) in 1,4-dioxane (3 mL) was stirred at 85° C. for 4 h. The reaction mixture was cooled to room temperature and volatiles were removed under reduced pressure. The crude mixture was purified by flash column chromatography (silica, mobile phase gradient: heptane to EtOAc) to afford 7-(3,4-dichlorobenzoyl)-2-[(propan-2-yl)amino]-3-[4-(4,4,5,5-tetramethyl-1,3,2-dioxaborolan-2-yl)phenyl]-5,6,7,8-tetrahydropyrido[3,4-d]pyrimidin-4(3H)-one (Intermediate I69) (325 mg, quant.) as a white foam.

### 3.1.16. Synthesis of Intermediate I70

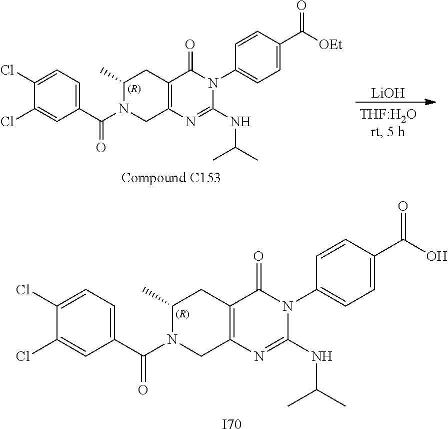

Compound C153 (430 mg, 791 μmol) was dissolved in THF (10 mL) and a solution of LIOH (38.0 mg, 1.58 mmol) in water (5 mL) was added. The reaction mixture was stirred at room temperature for 5 h. HCl (1 M in H₂O, 1.58 mL, 1.58 mmol) was added and the mixture was stirred for 10 min. The white solids were filtered off and washed with water and DIPE to afford (R)-4-(7-(3,4-dichlorobenzoyl)-2-(isopropylamino)-6-methyl-4-oxo-5,6,7,8-tetrahydropyrido[3,4-d]pyrimidin-3(4H)-yl)benzoic acid (Intermediate I70) (400 mg, 98%).

### 3.1.17. Synthesis of Intermediate I74

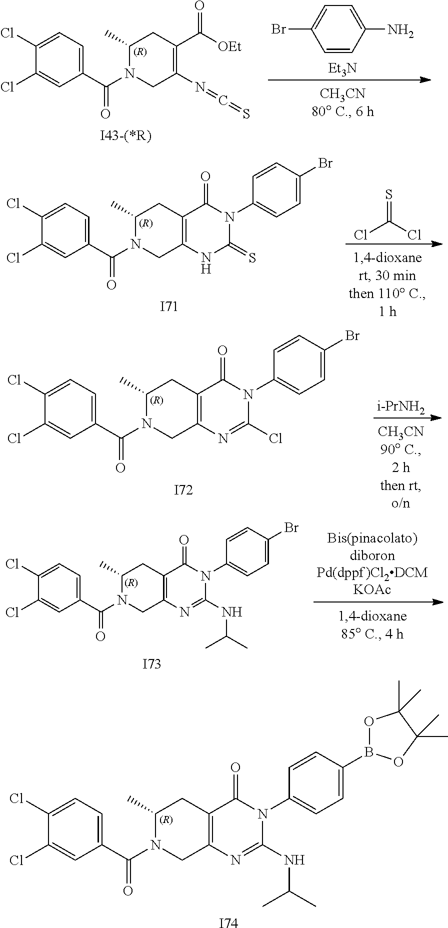

A reactor was charged with intermediate I43-(R) (5.00 g, 12.4 mmol), 4-bromoaniline (2.89 g, 16.2 mmol) and Et₃N (2.60 mL, 18.6 mmol) in dry CH₃CN (100 mL). The reaction mixture was stirred at 80° C. for 6 h. The reaction mixture was evaporated under reduced pressure and the crude mixture was purified by flash column chromatography (silica, mobile phase gradient: DCM/EtOAc, 100:0 to 50:50). The residue was triturated in EtOH and the solid was filtered off, washed with DIPE and dried under vacuum to afford (6R)-3-(4-bromophenyl)-7-(3,4-dichlorobenzoyl)-6-methyl-2-sulfanylidene-2,3,5,6,7,8-hexahydropyrido[3,4-d]pyrimidin-4(1H)-one (Intermediate I71) (5.9 g, 87%).

In a pressure tube, Intermediate I71 (2.00 g, 3.66 mmol) was dissolved in dry 1,4-dioxane (20.3 mL) and thiophosgene (0.39 mL, 5.12 mmol) was added. The reaction vessel was sealed under N₂ atmosphere. The reaction mixture was stirred at room temperature for 30 min and at 110° C. for 1 h. The mixture was cooled to room temperature and loaded on a silica cartridge. The mixture was purified by flash column chromatography (silica, mobile phase gradient: 0 to 100% EtOAc in heptane) to afford (6R)-3-(4-bromophenyl)-2-chloro-7-(3,4-dichlorobenzoyl)-6-methyl-5,6,7,8-tetrahydropyrido[3,4-d]pyrimidin-4(3H)-one (Intermediate I72) (1.86 g, 96%).

In a pressure tube, to a solution of intermediate I72 (2.53 g, 4.80 mmol) in dry CH₃CN (57 mL) under N₂ atmosphere was added isopropylamine (4.34 mL, 50.5 mmol) and the tube was sealed. The reaction mixture was stirred at 90° C. for 2 h, then at room temperature for 16 h. The reaction mixture was partially concentrated under reduced pressure. The white solid was filtered off, washed with water and DIPE and dried to afford (6R)-3-(4-bromophenyl)-7-(3,4-dichlorobenzoyl)-6-methyl-2-[(propan-2-yl)-amino]-5,6,7,8-tetrahydropyrido[3,4-d]pyrimidin-4(3H)-one (Intermediate I73) (2.09 g, 79%).

A microwave vial was charged with Intermediate I73 (500 mg, 0.91 mmol), bis(pinacolato)dibroron (462 mg, 1.82 mmol), KOAc (187 mg, 1.91 mmol), Pd(dppf)Cl₂.DCM (75.0 mg, 9.09 μmol) and dry 1,4-dioxane (3 mL). The vial was sealed and the reaction mixture was stirred at 85° C. for 4 h. The reaction mixture was cooled to room temperature and concentrated under reduced pressure. The crude mixture was purified by flash column chromatography (silica, mobile phase gradient: 0 to 100% EtOAc in heptane) to afford (6R)-7-(3,4-dichlorobenzoyl)-6-methyl-2-[(propan-2-yl)amino]-3-[4-(4,4,5,5-tetramethyl-1,3,2-dioxaborolan-2-yl)phenyl]-5,6,7,8-tetrahydropyrido[3,4-d]pyrimidin-4(3H)-one (Intermediate I74) (518 mg, 95%) as a white solid.

### 3.1.18. Synthesis of Intermediate I126

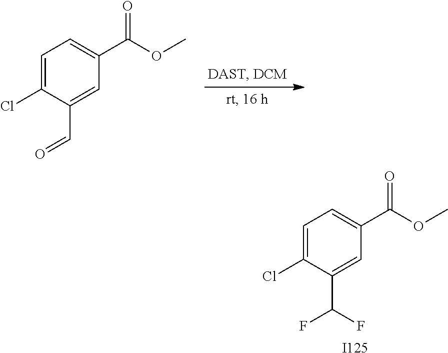

DAST (1.9 mL, 15.51 mmol) was added to a solution of methyl 4-chloro-3-formylbenzoate (1.02 g, 5.16 mmol) in dry DCM (25 mL). The reaction mixture was stirred at RT for 16 h. The rm was quenched with a saturated solution of NaHCO₃ aq., stirred 30 min, then the layers were separated. The aqueous layer was extracted with DCM (2×25 mL). The combined organic layers were dried over MgSO₄, filtered and evaporated under vacuum at 40° C. The crude was purified by column chromatography (heptane/EtOAc from 100/0 to 0/100) to afford the title compound methyl 4-chloro-3-(difluoromethyl)benzoate I125 (1.05 g, yield 92%) as a colourless oil.

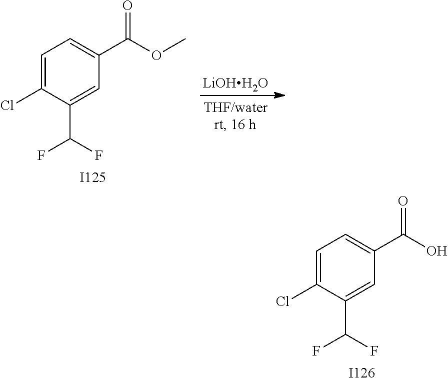

Lithium hydroxide monohydrate (412 mg, 9.82 mmol) was added in a mixture of methyl 4-chloro-3-(difluoromethyl)benzoate (1.05 g, 4.77 mmol) in THF (20 mL) and water (10 mL). The mixture was stirred at RT for 16 h. The THF was evaporated. EtOAc (10 mL) was added to the mixture. The organic layer was extracted, and the water layer was acidified with HCl 1 M in water to pH=1. The product was extracted with EtOAc (2×20 mL). The organic layers were combined, dried over MgSO₄, filtered and evaporated to afford the title compound I126 (808 mg, yield 82%) as a white powder.

### 3.1.19. Synthesis of Intermediate I127

Intermediates I127

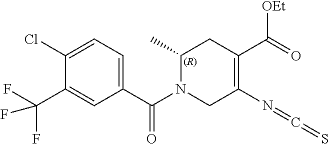

was synthetized following a similar procedure as for 143 starting from 4-chloro-3-(trifluoromethyl)benzoyl chloride.

### 3.1.20. Synthesis of Intermediate I130

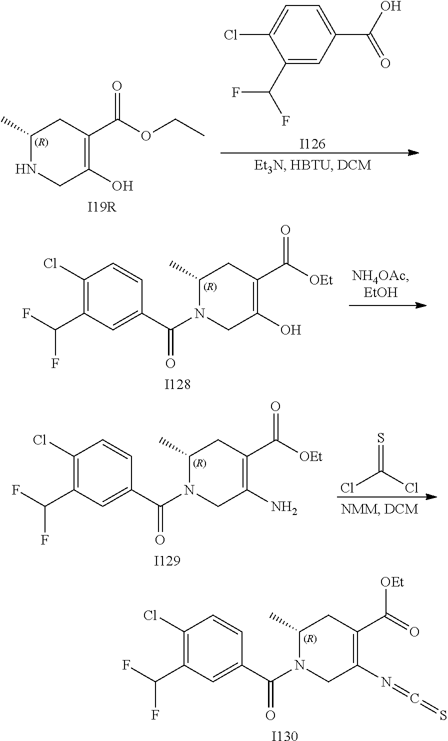

I19R (472 mg, 2.13 mmol) was added to a solution of I126 (400 mg, 1.94 mmol), dry triethylamine (1.3 mL, 9.62 mmol) and HBTU (1.1 g, 2.91 mmol) in dry DCM (20 mL). The mixture was stirred overnight at RT. The solvent was removed and the mixture was purified on silica column chromatography (heptane/EtOAc from 100/0 to 0/100) to afford I128 (553 mg, yield 70%) as a white powder.

I128 (511 mg, 1.258 mmol) was solubilised in EtOH (15 mL). Ammonium acetate (503 mg, 6.53 mmol) was added and the mixture was stirred overnight at RT. The volatiles were removed under reduced pressure and the product was washed with Na₂CO₃ aq. sat. (˜20 mL) and extracted in Me-THF (3×10 mL). The combined organic layers were dried over MgSO₄, filtered and concentrated in vacuo to afford the I129 (504 mg, yield quantitative) as a yellow oil and used as such in the next step.

I129 (500 mg, 1.247 mmol) and 4-methylmorpholine (0.45 mL, 4.08 mmol) were dissolved in dry DCM (4 mL). The mixture was cooled at −5° C. and thiophosgene (0.15 mL) was added dropwise at −5° C. The mixture was stirred 3 h in the bath. The product was purified on silica column chromatography (heptane/EtOAc from 100/0 to 0/50). The product fractions were concentrated in vacuo at 45° C. to afford the I130 498 mg, yield 96%) as an orange oil.

### 3.1.21. Synthesis of Intermediate I132

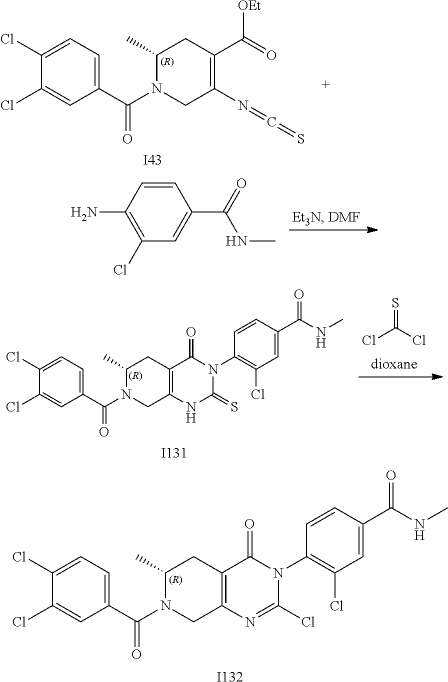

A VLT tube was charged with ethyl (2R)-1-(3,4-dichlorobenzoyl)-5-isothiocyanato-2-methyl-3,6-dihydro-2H-pyridine-4-carboxylate I43 (5.0 g, 12.53 mmol), 4-amino-3-chloro-N-methylbenzamide (2.97 g, 16.09 mmol), dry Et₃N (6 mL, 43.17 mmol), in dry DMF (5 mL). The mixture was stirred overnight at 95° C. The mixture was cooled and the mixture was concentrated in vacuo. The residue was purified by column chromatography (heptane/EtOAc from 100/0 to 0/100) to afford (R)-3-chloro-4-(7-(3,4-dichlorobenzoyl)-6-methyl-4-oxo-2-thioxo-1,4,5,6,7,8-hexahydropyrido[3,4-d]pyrimidin-3(2H)-yl)-N-methylbenzamide I131 (6.22 g, yield 60%) as a yellow oil.

Thiophosgene (1.4 mL, 17.716 mmol) was added to a solution of (R)-3-chloro-4-(7-(3,4-dichlorobenzoyl)-6-methyl-4-oxo-2-thioxo-1,4,5,6,7,8-hexahydropyrido[3,4-d]pyrimidin-3(2H)-yl)-N-methylbenzamide I131 (6.22 g, 7.52 mmol) in dry 1,4-dioxane (60 mL). The mixture was stirred 3 h at 100° C. The solvent was removed under reduced pressure. The residue was purified by column chromatography (heptane/EtOAc from 100/0 to 0/100) to afford I132 (1.21 g, yield 25%) as a yellow foam.

### 3.1.22. Synthesis of Intermediate I133

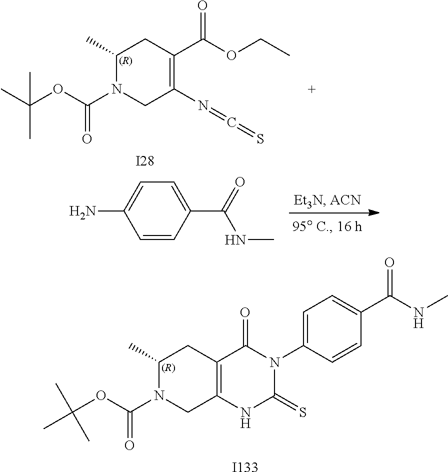

A large pressure tube was charged with 128 (23.44 g, 69.65 mmol), 4-amino-N-methylbenzamide (12.6 g, 83.90 mmol) and dry Et₃N (25 mL, 179.86 mmol) in dry CH₃CN (250 mL). The reaction mixture was stirred at 95° C. for 16 h. The solvent was removed at reduced pressure and the crude was washed with warm EtOAc (300 mL). The suspension was filtered to afford the title compound tert-butyl (R)-6-methyl-3-(4-(methylcarbamoyl)phenyl)-4-oxo-2-thioxo-2,3,4,5,6,8-hexahydropyrido[3,4-d]pyrimidine-7(1H)-carboxylate intermediate I133 (19.78 g, yield 65%) as a white powder.

### 3.1.23. Synthesis of Intermediate I134

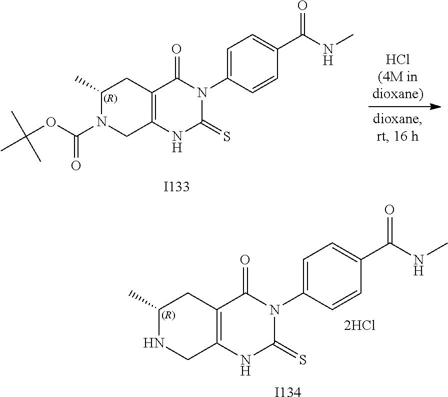

Tert-butyl (R)-6-methyl-3-(4-(methylcarbamoyl)phenyl)-4-oxo-2-thioxo-2,3,4,5,6,8-hexahydropyrido[3,4-d]pyrimidine-7(1H)-carboxylate I133 (306 mg, 0.66 mmol) was suspended in dry 1,4-dioxane (5 mL). HCl (1.8 mL, 4M in dioxane, 7.2 mmol) was added and the mixture was stirred at room temperature for 16 h. The rm was concentrated in vacuo to afford the title compound (R)-N-methyl-4-(6-methyl-4-oxo-2-thioxo-1,4,5,6,7,8-hexahydropyrido[3,4-d]pyrimidin-3(2H)-yl)-benzamide.2hydrochloride I134 (315 mg, yield quantitative) as a white powder.

### 3.1.24. Synthesis of Intermediate I138

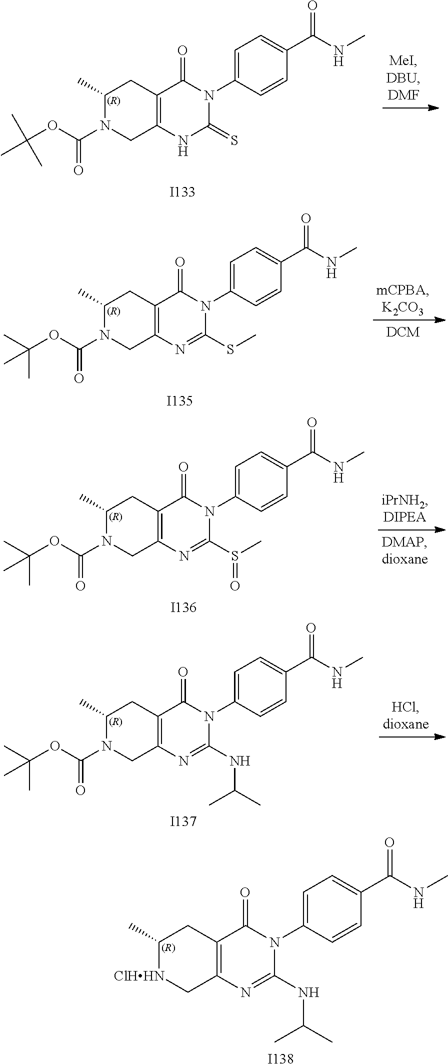

To a solution of tert-butyl (R)-6-methyl-3-(4-(methylcarbamoyl)phenyl)-4-oxo-2-thioxo-2,3,4,5,6,8-hexahydropyrido[3,4-d]pyrimidine-7(1H)-carboxylate I133 (17.6 g, 40.9 mmol) in DMF (400 mL) cooled at 0° C., were added DBU (9.16 mL, 61.3 mmol) and Mel (4.58 mL, 73.6 mmol) and the reaction mixture was stirred at 0° C. for 1 h 30. An aqueous saturated solution of NaHCO₃ (200 mL) and water (500 mL) were added. The aqueous layer was extracted with MeTHF (3×500 mL). The combined organic layers were washed with brine, dried over Na₂SO₄, filtered and concentrated under reduced pressure. The crude was purified by flash chromatography over silica gel (DCM/MeOH from 100/0 to 94/6) to afford tert-butyl (R)-6-methyl-3-(4-(methyl-carbamoyl)phenyl)-2-(methylthio)-4-oxo-4,5,6,8-tetrahydropyrido[3,4-d]pyrimidine-7(3H)-carboxylate I135 (18.8 g, yield 98%).

To a solution of tert-butyl (R)-6-methyl-3-(4-(methylcarbamoyl)phenyl)-2-(methylthio)-4-oxo-4,5,6,8-tetrahydropyrido[3,4-d]pyrimidine-7(3H)-carboxylate I135 (18.1 g, 40.7 mmol) in DCM (330 mL) cooled at 0° C. was added mCPBA (10.0 g, 44.8 mmol) and the reaction mixture was stirred 1.5 h at 0° C. Potassium carbonate (25.3 g, 183 mmol) was added and the reaction mixture was stirred at RT for 15 min. The reaction mixture was filtered and the filtrate was evaporated to afford tert-butyl (6R)-6-methyl-3-(4-(methylcarbamoyl)phenyl)-2-(methylsulfinyl)-4-oxo-4,5,6,8-tetrahydropyrido[3,4-d]pyrimidine-7(3H)-carboxylate I136 (23 g, yield quantitative) as a white solid, which was used as such for the next step.

Tert-butyl (6R)-6-methyl-3-(4-(methylcarbamoyl)phenyl)-2-(methylsulfinyl)-4-oxo-4,5,6,8-tetrahydropyrido[3,4-d]pyrimidine-7(3H)-carboxylate I136 (23.0 g, 40.5 mmol) was dissolved in dioxane (800 mL) and anhydrous isopropylamine (8.66 mL, 101 mmol), diisopropylethylamine (10.4 mL, 60.7 mmol) and DMAP (0.494 g, 4.05 mmol) were added and the reaction mixture was stirred for 18 h at 80° C. The reaction mixture was concentrated and the solid obtained was purified by flash chromatography (toluene/acetone from 100/0 to 50/50) a first pure fraction and a second non pure fraction. The second fraction was repurified by flash chromatography over silica gel (EtOAc/MeOH from 100/0 to 98/2). The two fraction were combined and co evaporated with EtOAc (50 mL) and dried under vacuum at 50° C. for 2 days to afford tert-butyl (R)-2-(isopropylamino)-6-methyl-3-(4-(methylcarbamoyl)phenyl)-4-oxo-4,5,6,8-tetrahydropyrido[3,4-d]pyrimidine-7(3H)-carboxylate I137 (9.8 g, 52%) as a yellow solid.

(R)-2-(isopropylamino)-6-methyl-3-(4-(methylcarbamoyl)phenyl)-4-oxo-4,5,6,8-tetrahydropyrido[3,4-d]pyrimidine-7(3H)-carboxylate I137 (4.55 g, 9.98 mmol) was suspended in dry 1,4-dioxane (100 mL). HCl (4M in dioxane) (35 mL, 4M in dioxane, 140 mmol) was added and the mixture was stirred overnight at room temperature. The reaction mixture was concentrated in vacuo. The product was washed with diethyl ether (100 mL×3) to afford (R)-4-(2-(isopropylamino)-6-methyl-4-oxo-5,6,7,8-tetrahydropyrido[3,4-d]pyrimidin-3(4H)-yl)-N-methylbenzamide hydrochloride I138 (1.01 g, yield quantitative) as a white powder and used as such.

### 3.1.25. Synthesis of Intermediate I140

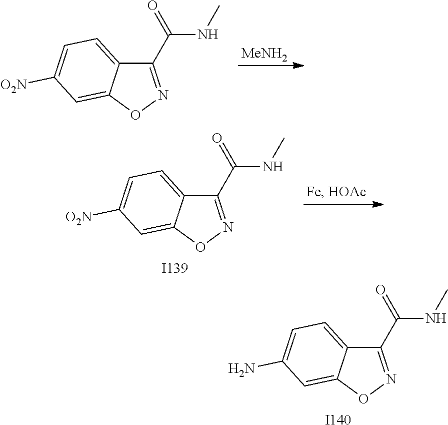 [SMILES: "MeNH2" ; CNC(=O)c1noc2cc([N+](=O)[O-])ccc12 ; "I139" ; "I140" ; CNC(=O)c1noc2cc([N+](=O)[O-])ccc12 ; CNC(=O)c1noc2cc(N)ccc12]

In a tube methylamine (10 mL, 2 M, 20 mmol) was added to methyl 6-nitro-1,2-benzisoxazole-3-carboxylate (1 g, 4.50 mmol). The tube was sealed, and the reaction mixture was heated at 60° C. for 16 h. The mixture was cooled and concentrated in vacuo. The residue was triturated in CH₃CN, filtered off and dried under vacuum to afford I139 (865 mg, yield 87%).

A tube was charged with I139 (865 mg, 3.91 mmol), iron (1.1 g mg, 19.55 mmol) in HOAc (15.9 mL). The mixture was heated at 60° C. for 30 min. A very sticky mixture was formed. The mixture was poured out in water and neutralized with sat. aq. Na₂CO₃. The mixture was extracted with Me-THF (3×). The organic layer was separated, dried over MgSO₄, filtered and concentrated in vacuo. The residue was purified on silica column chromatography (heptane/EtOAc-EtOH (3-1) from 100/0 to 0/100). The resulting product was triturated in DIPE, filtered off and dried to afford I140 (293 mg, yield 24%) as a yellow solid which was used as such in the next step.

### 3.1.26. Synthesis of Intermediate I144

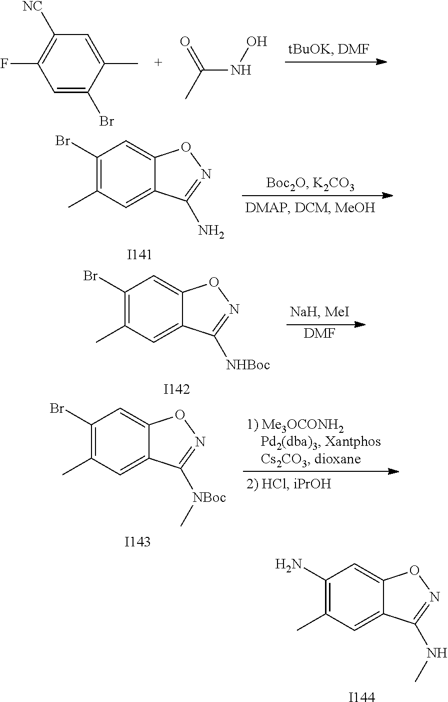

Acetohydroxamic acid (2.63 g, 35.04 mmol) was dissolved in DMF (40 mL) and tBuOK (3.93 g, 35.04 mmol) was added. The mixture was stirred at RT for 30 min. 4-bromo-2-fluoro-5-methylbenzonitrile (5 g, 23.36 mmol) was added in one portion and the mixture was stirred 16h at RT. The mixture was poured out in 100 ml sat. NH₄Cl solution. The mixture was extracted with Me-THF and the organic layer was separated, dried over MgSO₄, filtered and concentrated in vacuo. The residue was purified by column chromatography (heptane/EtOAc from 100/0 to 0/60). The obtained product was triturated in DIPE, filtered off and dried under vacuum to afford I141 (2.9 g, yield 55%) as white solid.

I141 (2.9 g, 12.77 mmol) was dissolved in DCM (30 mL). Di-tert-butyl dicarbonate (14 mL, 2 M, 28.098 mmol) was added. DMAP (156 mg, 1.27 mmol) was added in one portion. The mixture was stirred at RT for 1 h. The mixture was concentrated in vacuo. The residue was dissolved in MeOH (49 mL) and K₂CO₃ (3.5 g, 25.54 mmol) was added. The reaction mixture was refluxed for mixture before being cooled down and concentrated in vacuo. The residue was portioned between water and DCM. The mixture was neutralized with 1N HCl solution. The organic layer was separated, dried over MgSO₄, filtered and concentrated in vacuo. The residue was purified on silica column chromatography (heptane/EtOAc from 100/0 to 0/100) to afford I142 (3.4 g, yield 81%) as a white solid.

I142 (3.4 g, 10.39 mmol) was dissolved in DMF (30 mL) under inert atmosphere. NaH (60% dispersion in mineral oil) (498 mg, 12.47 mmol) was added and the mixture was stirred at RT for 10 min. Mel (0.71 mL, 11.43 mmol) was added dropwise and the mixture was stirred at RT for 1 h. The mixture was poured out in ice water and extracted with Me-THF. The organic layer was separated, dried over MgSO₄, filtered and concentrated in vacuo. The residue was purified on silica column chromatography (heptane/EtOAc from 100/0 to 0/100), The resulting product was triturated in DIPE, filtered off and dried under vacuum to afford I143 (2.9 g, yield 82%) as a yellow solid.

A 20 mL microwave vial was charged with I143 (1 g, 2.93 mmol), tert-butyl carbamate (412 mg, 3.52 mmol), Cs₂CO₃ (1.91 g, 5.86 mmol) in dry 1,4-dioxane (15 mL). The mixture was purged with N₂ for 5 min. Xantphos (153 mg, 0.264 mmol) and Pd₂(dba)₃ (91 mg, 0.10 mmol) were added and the vial was sealed. The mixture was heated at 120° C. for 16 h. The mixture was cooled and filtered over decalite. The filtrate was concentrated in vacuo and was dissolved in i-PrOH (19 mL) and HCl (4.9 mL, 6 M in i-PrOH, 29.3 mmol) was added. The mixture was heated at reflux for 30 min. The mixture was cooled down and concentrated in vacuo. The residue was dissolved in water and neutralized with sat. aq. Na₂CO₃. The mixture was extracted with Me-THF and the organic layer was separated, dried over MgSO₄, filtered and concentrated in vacuo. The residue was purified silica column chromatography (heptane/EtOAc from 100/0 to 0/100) to afford I144 (342 mg, yield 66%) as a solid.

### 3.1.27. Synthesis of Intermediate I147

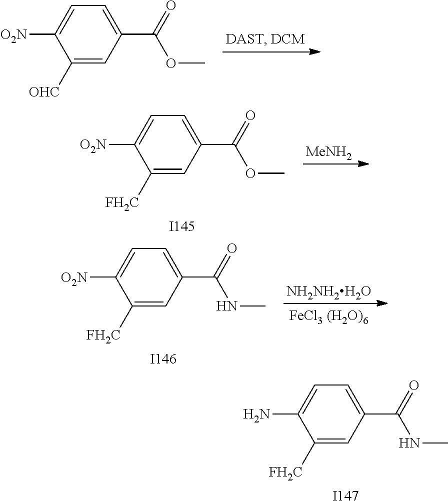

To a solution methyl 3-formyl-4-nitrobenzoate (2 g, 9.28 mmol) at 0° C. under inert atmosphere was added DAST (1.47 mL, 11.13 mmol) dropwise over 5 min. The reaction mixture was allowed to stir at 0° C. for 2 h followed by 15 min at RT. 20 mL sat. aq. NaHCO₃ solution was added to stop the reaction and DCM was added. The organic phase was dried over Na₂SO₄, filtered and concentrated in vacuo. The residue was purified by column chromatography (heptane/EtOAc from 100/0 to 0/100) to afford I145 (5.1 g, yield 95%) a yellowish oil.

A flask was charged with I145 (4 g, 17.3 mmol) and methylamine (40 mL) was added. The mixture was heated at 60° C. for 16 h. The mixture was concentrated in vacuo and the residue was purified on silica column chromatography (heptane/EtOAc from 100/0 to 0/100). The resulting product was triturated in DIPE, filtered off and dried to afford I146 (3.5 g, yield 88%) as a white solid.

To a solution of I146 (500 mg, 2.17 mmol) in EtOH (10 mL), were added hydrazine hydrate (1:1) (800 μL, 8.22 mmol), activated carbon (100 mg) and iron(III) chloride hexahydrate (45 mg, 0.16 mmol). The mixture was refluxed for 4 h. The reaction mixture was filtered. The filtrate was diluted with EtOAc (80 mL) and washed with water (2×25 mL) and saturated aqueous NaCl solution (25 mL), dried over anhydrous MgSO₄ and concentrated in vacuo. The residue was purified by column chromatography (heptane/EtOAc-EtOH (3-1) from 100/0 to 0/100) to afford I147 (314 mg, yield 72%).

### 3.1.28. Synthesis of Intermediate I148

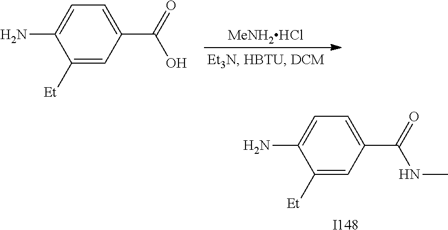

A tube was charged with 4-amino-3-ethylbenzoic acid (1 g, 6.05 mmol), HBTU (2.76 g, 7.26 mmol) and triethylamine (4.2 mL) in dry DCM (31 mL). The solution was stirred for 15 min. at RT and then methylamine hydrochloride (0.6 g, 9.08 mmol) was added and the stirring was continued for 16 h. The reaction mixture directly purified on silica column chromatography (heptane/EtOAc from 100/0 to 0/100) to afford a crude of I148 (1.6 g, yield quantitative) as a clear orange oil.

### 3.1.29. Synthesis of Intermediate I152

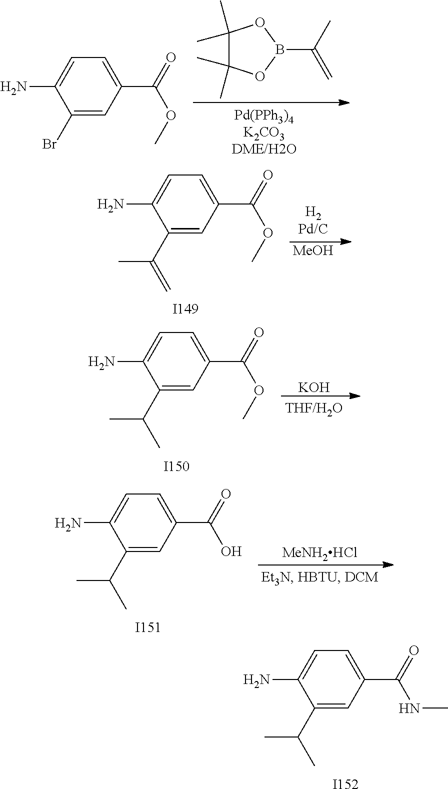

Isoprene boronicacid pinacolester (5 mL, 26.6 mmol) was added to a solution of methyl 4-amino-3-bromobenzoate (5.03 g, 21.88 mmol), Pd(PPh₃)₄ (2.6 g, 2.25 mmol) and potassium carbonate (6.2 g, 44.7 mmol) in dry DME (90 mL) and water (10 mL). The mixture was stirred overnight at 120° C. and the solvent was removed under reduced pressure. The residue was washed with water (˜200 mL) and extracted with DCM (3×50 mL). The different organic phases were combined, dried over MgSO₄, filtered and evaporated under reduced pressure. The residue was purified on silica column chromatography (heptane/EtOAc from 100/0 to 0/100) to afford the I149 (2.73 g, yield 57%) as an orange oil.

A flask containing a mixture of I149 (2.73 g, 12.56 mmol), Pd/C (10%) (1.34 g, 1.26 mmol) in MeOH (152 mL) was charged with hydrogen and stirred hydrogenated at RT for 48 h. The reaction mixture was filtered on decalite and the filtrate was evaporated to afford the I150 as a dark orange oil which was used as such in the next step.

KOH (1.58 g, 28.13 mmol) was added in a mixture of I150 (2.64 g, 13.50 mmol) in THF (40 mL) and water (25 mL)). The reaction mixture was stirred overnight at RT (no reaction). KOH (˜2 g) was added and the mixture was heated at 75° C. to 2 days. (complete conversion). THF was evaporated. The pH was acidified until pH=1 with HCl (1 M in water) and the suspension was stirred for 1 h. The solid was filtered, washed with water and solubilised in a mixture of EtOH/DCM (1/4). The filtrate was dried over MgSO₄, filtered concentrated under reduced pressure to afford I151 as a light red powder which was used as such in the next step.

Methylamine hydrochloride (1.45 g, 21.48 mmol) was added to a solution of I151 (3.06 g, 14.19 mmol), dry TEA (15 mL, 107 mmol) and HBTU (6.53 g, 17.23 mmol) in dry DCM (90 mL). The mixture was stirred 16 h at RT. The solvent was removed under reduced pressure and the product was purified on silica column chromatography (heptane/EtOAc from 100/0 to 0/100) to afford the I152 (1.08 g (100% pure), yield 39%) and (1.92 g (88% pure), yield 62%)) as orange oils.

### 3.1.30. Synthesis of Intermediate I154

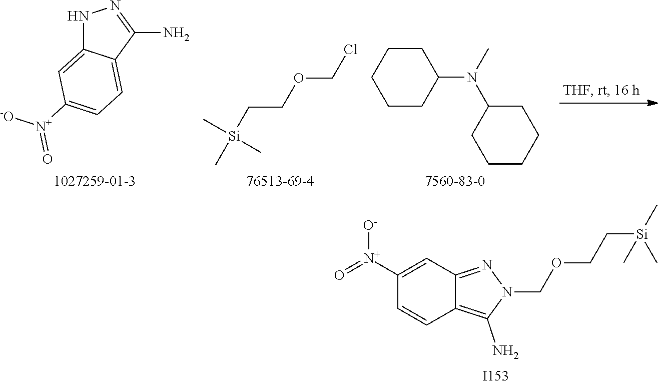

A 100 mL flask was charged with 6-nitro-1H-indazol-3-amine (1.5 g, 8.42 mmol) and N,N-dicyclohexylmethylamine (2.2 mL, 10.10 mmol) in dry THF (50 mL). 2-(trimethylsilyl)ethoxymethyl chloride (1.9 mL, 10.10 mmol) was added dropwise and the reaction mixture was stirred at RT for 16 h. The mixture was diluted with Me-THF and quenched with 100 mL 0.5 N NaOH. The organic layer was separated, dried over MgSO₄, filtered and concentrated in vacuo. The residue was purified by column chromatography (heptane/EtOAc from 100/0 to 0/100) to afford the title compound 6-nitro-2-((2-(trimethylsilyl)ethoxy)methyl)-2H-indazol-3-amine 1.52 g, yield 59%) as a red semi solid.

The following steps to obtain I154

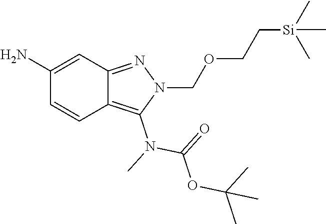 [SMILES: CN(C(=O)OC(C)(C)C)c1c2ccc(N)cc2nn1COCC[Si](C)(C)C]

were similar to the synthesis of tert-butyl (6-aminobenzo[d]isoxazol-3-yl)(methyl)carbamate I144.

### 3.1.31. Synthesis of Intermediate I159

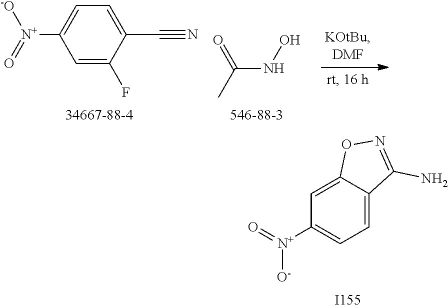

Acetohydroxamic acid (678 mg, 9.03 mmol) was dissolved in DMF (13.7 mL). KOtBu (675.5 mg, 6.02 mmol) was added and the mixture was stirred at RT for 30 min. 2-Fluoro-4-nitrobenzonitrile (1 g, 6.02 mmol) was added and the mixture was further stirred at RT for 16 h. The mixture was concentrated in vacuo and water was added. The precipitated product was filtered off and washed two times with CH₃CN. The product was dried under vacuum to become the title compound 6-nitrobenzo[d]isoxazol-3-amine I155 (481 mg, yield 45%) as a pale yellow solid.

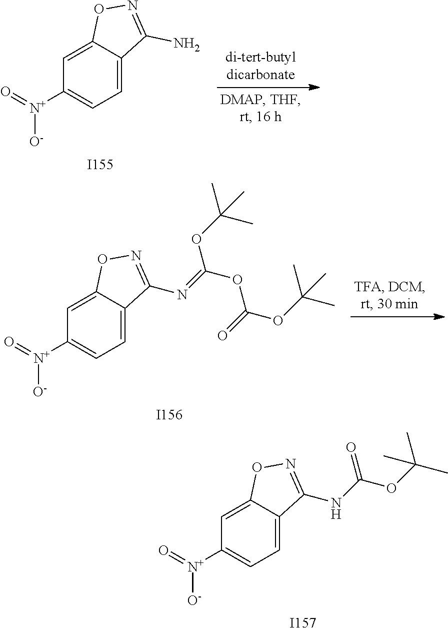 [SMILES: "I155" ; "I156" ; "I157" ; Nc1noc2cc([N+](=O)[O-])ccc12 ; CC(C)(C)OC(=O)O/C(=N/c1noc2cc([N+](=O)[O-])ccc12)OC(C)(C)C ; CC(C)(C)OC(=O)Nc1noc2cc([N+](=O)[O-])ccc12]

6-Nitrobenzo[d]isoxazol-3-amine I155 (481 mg, 2.69 mmol) and DMAP (65.6 mg, 0.54 mmol) was dissolved in THF (10 mL). Di-tert-butyl dicarbonate (2.7 mL, 2 M, 5.37 mmol) was added and the mixture was stirred at RT for 16 h. The mixture was concentrated in vacuo. The residue was purified by column chromatography (heptane/EtOAc from 100/0 to 0/100). The resulting compound (801 mg) was dissolved in DCM (5 mL) and TFA (323 μL, 1.49 g/mL, 4.22 mmol) was added at RT. The mixture was stirred at RT for 30 min. The mixture was washed with sat. NaHCO₃ solution and the organic layer was separated, dried over MgSO₄, filtered and concentrated in vacuo. The product was triturated in DIPE, filtered off and dried under vacuum to become the title compound tert-butyl (6-nitrobenzo[d]isoxazol-3-yl) carbamate I157 (501 mg, yield 67%) as a white solid.

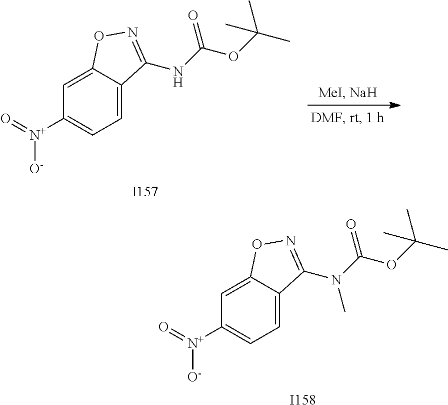 [SMILES: "I157" ; "I158" ; CC(C)(C)OC(=O)Nc1noc2cc([N+](=O)[O-])ccc12 ; CN(C(=O)OC(C)(C)C)c1noc2cc([N+](=O)[O-])ccc12]

Tert-butyl (6-nitrobenzo[d]isoxazol-3-yl) carbamate I157 (2 g, 7.162 mmol) was dissolved in DMF (21 mL) under N₂ atmosphere. NaH (60% dispersion in mineral oil) (344 mg, 8.59 mmol) was added and the mixture was stirred at RT for 10 min. Mel (0.49 mL, 7.88 mmol) was added dropwise and the mixture was stirred at RT for 1 h. The mixture was poured out in ice water and stirred for 15 min. The product was filtered off and washed with water and dried under vacuum. The product was triturated in DIPE, filtered off and dried under vacuum to become the title compound tert-butyl methyl(6-nitrobenzo[d]isoxazol-3-yl) carbamate I158 (2.1 g, yield quantitative) as a yellow solid.

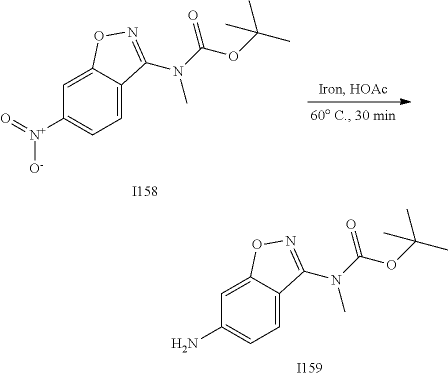 [SMILES: "I158" ; "I159" ; CN(C(=O)OC(C)(C)C)c1noc2cc([N+](=O)[O-])ccc12 ; CN(C(=O)OC(C)(C)C)c1noc2cc(N)ccc12]

A reaction tube was charged with tert-butyl methyl(6-nitrobenzo[d]isoxazol-3-yl) carbamate (2.1 g, 7.16 mmol), iron (2.0 g, 35.80 mmol) in HOAc (29 mL). The mixture was heated at 60° C. for 30 min. The mixture was cooled and concentrated in vacuo. The residue was diluted with DCM and filtered over decalite. The filtrate was washed with sat. Na₂CO₃ solution. The organic layer was separated, dried over MgSO₄, filtered and concentrated in vacuo. The residue was purified by column chromatography (Heptane/EtOAc-EtOH (3:1) from 100/0 to 0/100). The product fractions were collected and concentrated in vacuo to become the title compound tert-butyl (6-aminobenzo[d]isoxazol-3-yl)(methyl)carbamate I159 (1.3 g, yield 69%) as a solid.

### 3.1.32. Synthesis of Intermediate I160

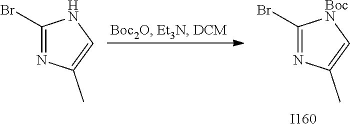

2-bromo-4-methyl-1H-imidazole (500 mg, 3.11 mmol) was dissolved in DCM (20 mL) under inert atmosphere. Et₃N (0.52 mL, 3.73 mmol) and BOC-anhydride (0.73 mL, 3.42 mmol) were added dropwise. The mixture was stirred overnight. DCM/MeOH (30 mL) were added and the organic layer was washed twice with an aqueous solution of 10% of K₂CO₃ (15 mL). The organic phases were collected and washed with brine, dried over MgSO₄, and concentrated under reduced pressure. The residue was purified on silica column chromatography using DCM/MeOH to afford I160 (760 mg, 90% yield).

### 3.2. Final Products Syntheses

### Procedure A

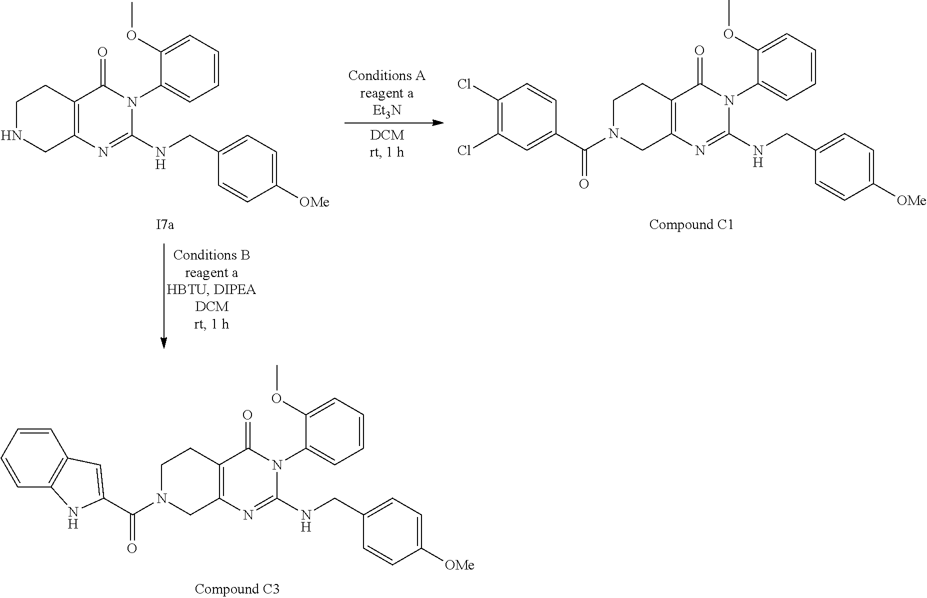 [SMILES: COc1ccc(CNc2nc3c(c(=O)n2-c2ccccc2OC)CCNC3)cc1 ; COc1ccc(CNc2nc3c(c(=O)n2-c2ccccc2OC)CCN(C(=O)c2ccc(Cl)c(Cl)c2)C3)cc1 ; COc1ccc(CNc2nc3c(c(=O)n2-c2ccccc2OC)CCN(C(=O)c2cc4ccccc4[nH]2)C3)cc1]

### Conditions A

A mixture of Intermediate I7a (106 mg, 0.23 mmol), 3,4-dichlorobenzoyl chloride (reagent a) (51.5 mg, 0.24 mmol) and Et₃N (0.16 mL, 1.14 mmol) in DCM (30 ml-) was stirred at room temperature for 1 h. The mixture was washed with a saturated aqueous solution of NaHCO₃ (50 mL), dried (Na₂SO₄), filtered and concentrated under reduced pressure. The residue was purified by flash column chromatography (silica, mobile phase gradient: 10 to 100% EtOAc in heptane). The product was dried at 50° C. under vacuum overnight to give Compound C1 (96 mg, 75%) as a clear resin.

### Conditions B

A mixture of Intermediate I7a (65.0 mg, 0.14 mmol), 3-chloro-4-fluorobenzoic acid (reagent a) (25.6 mg, 147 μmol), DIPEA (96.3 μL, 0.56 mmol) and HBTU (53.3 mg, 0.14 mmol) in DCM (0.83 mL) was stirred at room temperature for 1 h. The reaction mixture was purified by flash column chromatography (silica, mobile phase gradient: 40 to 100% EtOAc in heptane). The residue was dissolved in warm i-PrOH (1 mL) and the solution was acidified with HCl (6M in i-PrOH). A precipitate was observed. The mixture was diluted slowly with DIPE (25 mL). The white powder was filtered off and dried overnight under vacuum to afford Compound C3 as a HCl salt (65 mg, 79%).

### Procedure B

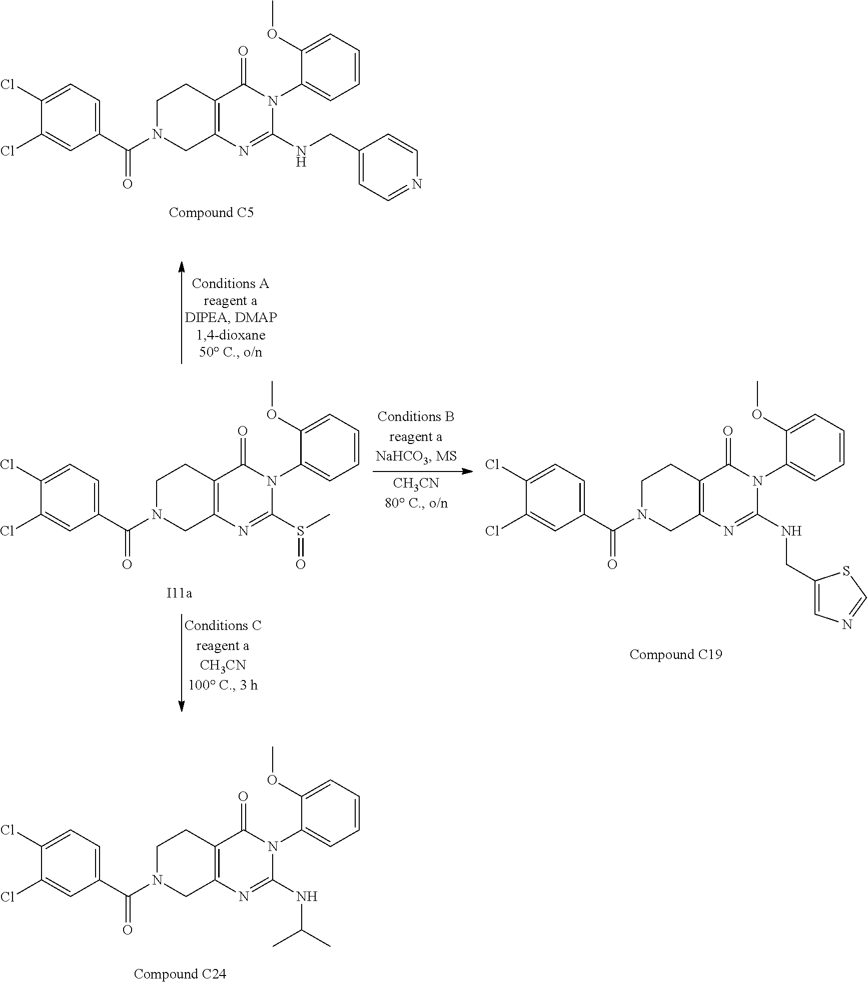 [SMILES: COc1ccccc1-n1c(NCc2ccncc2)nc2c(c1=O)CCN(C(=O)c1ccc(Cl)c(Cl)c1)C2 ; COc1ccccc1-n1c(S(C)=O)nc2c(c1=O)CCN(C(=O)c1ccc(Cl)c(Cl)c1)C2 ; COc1ccccc1-n1c(NCc2cncs2)nc2c(c1=O)CCN(C(=O)c1ccc(Cl)c(Cl)c1)C2 ; COc1ccccc1-n1c(NC(C)C)nc2c(c1=O)CCN(C(=O)c1ccc(Cl)c(Cl)c1)C2]

### Conditions A

A mixture of Intermediate I11a (125 mg, 254 μmol), 4-picolylamine (reagent a) (47 μL, 0.46 mmol), DIPEA (44 μL, 0.25 mmol) and DMAP (2.8 mg, 0.023 mmol) in 1,4-dioxane (5 mL) was stirred at 50° C. overnight. The reaction mixture was cooled to room temperature and concentrated under reduced pressure. The crude mixture was purified via preparative HPLC (Stationary phase: RP XBridge Prep C18 OBD-10 μm, 50×150 mm, Mobile phase: 0.25% NH₄HCO₃ solution in water, CH₃CN). The residue was dissolved in MeOH and concentrated under reduced pressure. The residue was triturated in DIPE, filtered off and dried under vacuum to afford Compound C5 (66 mg, 48%) as a white solid.

### Conditions B

To a mixture of Intermediate I11a (125 mg, 254 μmol), thiazol-5-ylmethanamine (reagent a) (68.8 mg, 457 μmol) and NaHCO₃ (53.3 mg, 0.64 mmol) in dry CH₃CN (4 mL) was added molecular sieves 4A (1 g). The reaction mixture was stirred at 80° C. overnight, cooled to room temperature and concentrated under reduced pressure. The crude mixture was purified via Prep HPLC (Stationary phase: RP XBridge Prep C18 OBD-10 μm, 50×150 mm, Mobile phase: 0.25% NH₄HCO₃ solution in water, CH₃CN). The residue was dissolved in MeOH and concentrated under reduced pressure. The residue was triturated in DIPE, filtered off and dried under vacuum to afford Compound C19 (41 mg, 30%) as a white solid.

DMAP was used instead of NaHCO₃ in the synthesis of Compounds C₂₀ and C₂₂. Molecular sieves were not used in the synthesis of Compound C25.

### Conditions C

A microwave tube was charged with Intermediate I11a (125 mg, 254 μmol), isopropylamine (reagent a) (39.3 μL, 457 μmol) and CH₃CN (3 mL), and sealed. The reaction mixture was stirred at 100° C. for 3 h, cooled to room temperature and concentrated under reduced pressure. The crude mixture was purified via Prep HPLC (Stationary phase: RP XBridge Prep C18 OBD-10 μm, 50×150 mm, Mobile phase: 0.25% NH₄HCO₃ solution in water, CH₃CN). The residue was dissolved in MeOH and concentrated under reduced pressure. The residue was triturated in heptane, filtered off and dried under vacuum to afford Compound C24 (14 mg, 11%) as a white solid.

### Procedure C

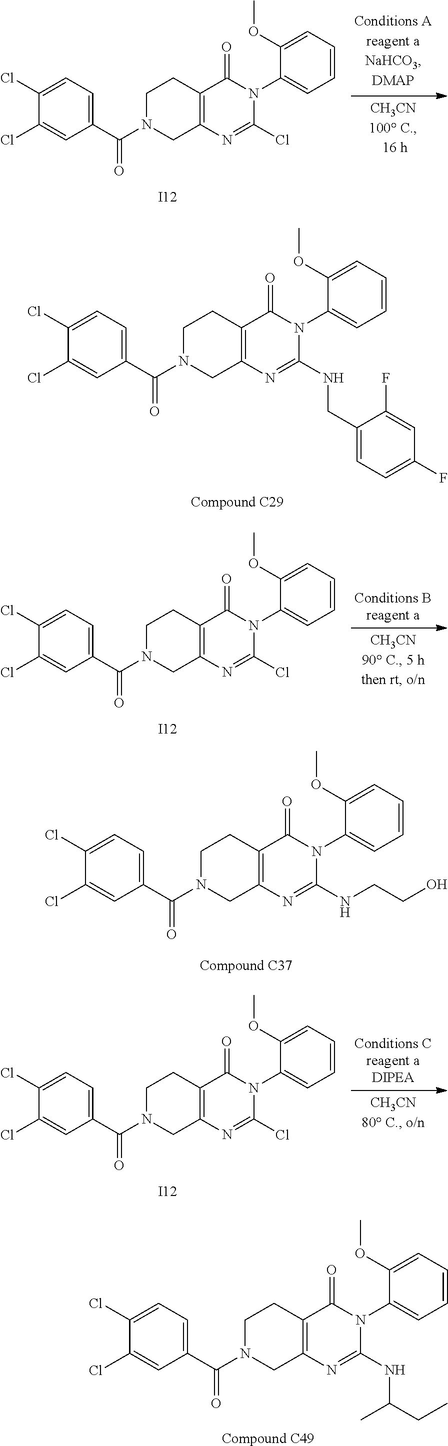

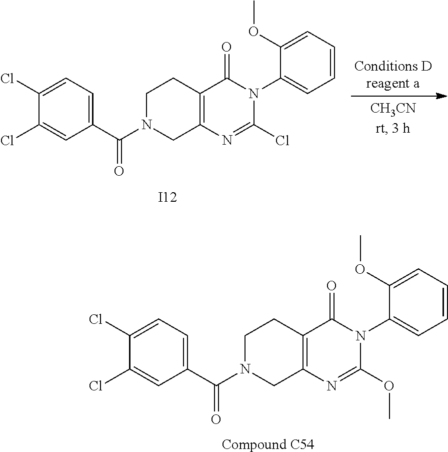

### Conditions A

A microwave tube was charged with Intermediate I12 (125 mg, 0.27 mmol), 2,4-difluorobenzylamine (reagent a) (59 mg, 0.40 mmol), NaHCO₃ (45 mg, 0.54 mmol), DMAP (2.9 mg, 0.024 mmol) and dry CH₃CN (2 mL), and sealed. The reaction mixture was stirred at 100° C. for 16 h. The reaction mixture was cooled to room temperature and filtered over decalite. The filtrate was concentrated under reduced pressure and the crude mixture was purified via preparative HPLC (Stationary phase: RP XBridge Prep C18 OBD-10 μm, 50×150 mm, Mobile phase: 0.25% NH₄HCO₃ solution in water, MeOH). The residue was dissolved in MeOH and concentrated under reduced pressure. The residue was triturated in DIPE, filtered off and dried under vacuum to afford Compound C29 (34 mg, 22%) as a white solid.

### Conditions B

To a solution of Intermediate I12 (100 mg, 215 μmol) in dry CH₃CN (2.56 mL) under N₂ atmosphere was added ethanolamine (reagent a) (0.13 mL, 2.15 mmol). The reaction mixture was stirred at 90° C. for 5 h, then at room temperature for 16 h. Volatiles were removed under reduced pressure. The crude mixture was purified by flash column chromatography (silica, mobile phase gradient: 0 to 100% EtOAc in DCM, then 0 to 10% MeOH in DCM) to afford Compound C37 (84 mg, 80%) as a white solid.

The reaction mixture was stirred at 80° C. for 16 hours in the synthesis of Compounds C46, C47, C48 and C62.

The reaction mixture was stirred at 110° C. for 16 hours in the synthesis of Compound C101.

The reaction was performed under neat conditions for the synthesis of Compounds C38, C46, C57, C86 and C101.

### Conditions C

To a solution of Intermediate I12 (100 mg, 215 μmol) in dry CH₃CN (2.56 mL) under N₂ atmosphere were added s-butylamine (reagent a) (87 μL, 0.86 mmol) and DIPEA (74.2 μL, 0.43 mmol). The reaction mixture was stirred at 80° C. for 16 h. Volatiles were removed under reduced pressure. The crude mixture was purified by flash column chromatography (silica, mobile phase gradient: 0 to 10% MeOH in DCM). A second purification was performed via Prep HPLC (Stationary phase: RP XBridge Prep C18 OBD-10 μm, 30×150 mm, Mobile phase: 0.25% NH₄HCO₃ solution in water, CH₃CN) to give Compound C49 (65 mg, 60%) as a white solid.

Et₃N was used instead of DIPEA in the synthesis of Compound C32.

### Conditions D

To a solution of Intermediate I12 (100 mg, 215 μmol) in dry CH₃CN (2.56 mL, 49.1 mmol) under N₂ atmosphere was added NaOMe (reagent a) (0.5M in MeOH, 2 mL, 1 mmol). The reaction mixture was stirred at room temperature for 3 h. The pH was neutralized with 3M aqueous solution of HCl. The mixture was concentrated under reduced pressure. The crude mixture was purified via Prep HPLC (Stationary phase: RP XBridge Prep C18 OBD-10 μm, 30×150 mm, Mobile phase: 0.25% NH₄HCO₃ solution in water, CH₃CN) to afford Compound C54 (42 mg, 42%) as a white solid.

### Procedure D

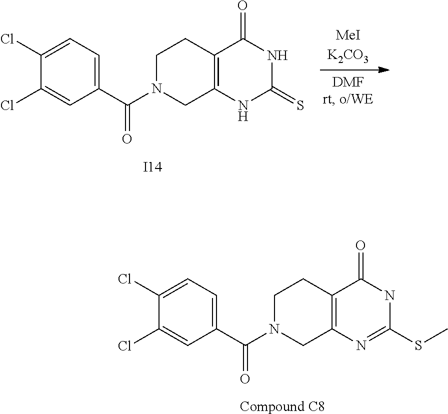

Mel (1.75 g, 12.4 mmol) was added to a mixture of Intermediate I14 (2.2 g, 6.18 mmol) and K₂CO₃ (854 mg, 6.18 mmol) in DMF (200 mL) The reaction mixture was stirred at room temperature over the weekend and concentrated under reduced pressure. The residue was dissolved in DCM (250 mL). The solution was washed with a saturated aqueous solution of NaHCO₃ (200 mL), dried (MgSO₄), filtered and concentrated under reduced pressure. The residue was dissolved in boiling MeOH (300 mL) and filtered while still hot. Water (200 mL) was added slowly to the filtrate and the product was allowed to crystallize. The precipitate was filtered and dried overnight under vacuum at 50° C. yielding Compound C6 (1.51 g).

### Procedure E

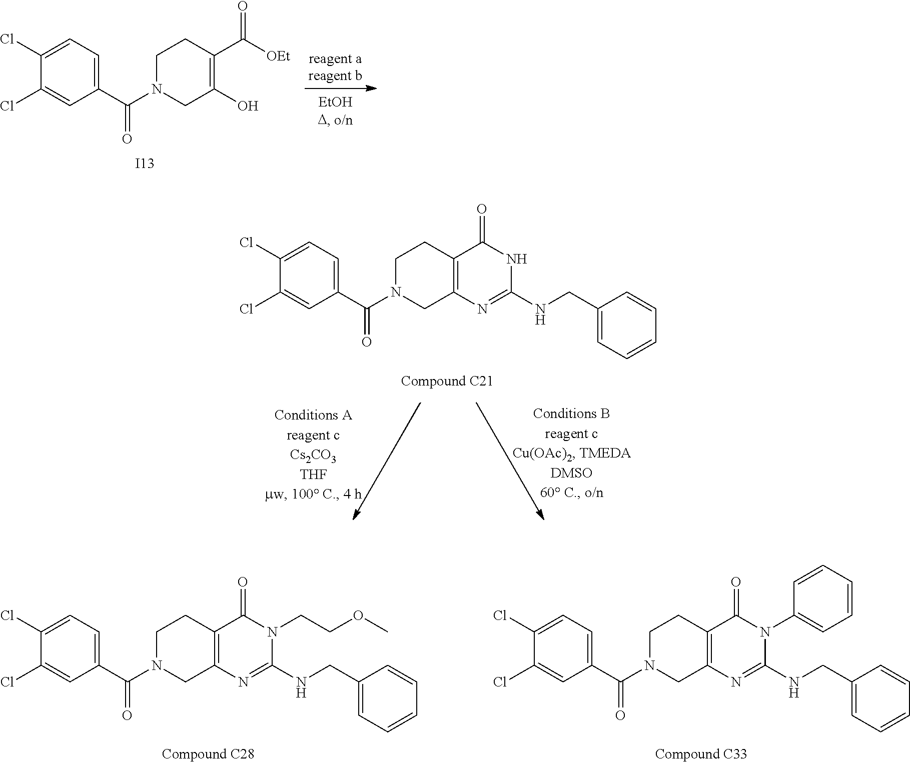

A mixture of Intermediate I13 (1.34 g, 3.91 mmol), N-benzylguanidine (reagent a) (612 mg, 4.1 mmol) and t-BuOK (reagent b) (974 mg, 8.59 mmol) in EtOH (50 mL) was refluxed overnight. The reaction mixture was cooled to room temperature and concentrated under reduced pressure. The residue was suspended in water (50 mL), HOAc (0.19 mL, 3.36 mmol) was added and the mixture was stirred for 15 min. The white precipitate was filtered off, rinsed with water and dried under vacuum at 60° C. for 2 h. The powder was dissolved in warm EtOH/DMF (90:10, 100 mL), filtered and purified by flash column chromatography (silica, mobile phase gradient: 0 to 100% heptane in EtOAc/EtOH (3:1), then 0 to 100% EtOAc/EtOH (3:1) in i-PrOH). A second purification by flash column chromatography was performed (silica, mobile phase gradient: 10 to 100% EtOAc in heptane, then 0 to 25% EtOH in EtOAc/EtOH (3:1)). The product was dried under vacuum at 60° C. to afford Compound C21 (476 mg, 28%) as a white powder.

The reaction mixture was stirred at room temperature overnight for the synthesis of Compounds C42, C63 and C89.

### Conditions A

A mixture of Compound C21 (123 mg, 287 μmol), 2-bromoethyl methyl ether (reagent c) (47.8 mg, 0.34 mmol) and Cs₂CO₃ (467 mg, 1.43 mmol) in THF (10 mL) was heated in a microwave at 100° C. for 4 h. An additional amount of 2-bromoethyl methyl ether (reagent c) (48 mg, 0.34 mmol) and Cs₂CO₃ (467 mg, 1.43 mmol) was added and the reaction mixture was heated at 100° C. for 1 h twice. The reaction mixture was filtered and concentrated under reduced pressure. The crude mixture was purified by flash column chromatography (silica, mobile phase gradient: 10 to 100% EtOAc in heptane, then 0 to 100% EtOAc/EtOH (3:1) in EtOAc). The residue was dissolved in i-PrOH (1 mL) and the solution was acidified with HCl (6M in i-PrOH). On the addition of DIPE precipitation occurred. The precipitate was filtered off and dried under vacuum at 45° C. overnight to give Compound C28 (20.3 mg, 14%) as a HCl salt and as a white powder.

### Conditions B

A mixture of Compound C21 (49.0 mg, 114 μmol), TMEDA (13.4 mg, 114 μmol), Cu(OAc)₂ (20.9 mg, 114 μmol) and phenylboronic acid (reagent c) (13.9 mg, 114 μmol) in DMSO (0.5 mL, 6.99 mmol) was stirred at 60° C. After 1 h, an additional amount of phenylboronic acid (reagent c) (13.9 mg, 114 μmol) was added and the reaction mixture was stirred at 60° C. overnight. The mixture was loaded on a silica cartridge and purified by flash column chromatography (silica, mobile phase gradient: 50 to 100% EtOAc in heptane, then 0 to 25% EtOH in EtOAc). The product was dried under vacuum overnight to afford Compound C33 (20 mg) as a clear oil.

### Procedure F

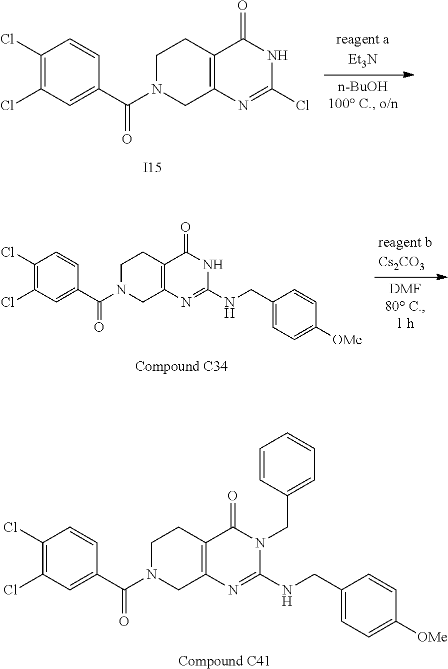

A microwave tube was charged with Intermediate I15 (1.37 g, 3.83 mmol), 4-methoxybenzylamine (reagent a) (1.0 mL, 7.7 mmol), Et₃N (1.06 mL, 7.7 mmol) and n-BuOH (15 mL). The tube was sealed and the reaction mixture was stirred at 100° C. overnight. The volatiles were removed under reduced pressure. The crude mixture was purified by flash column chromatography (silica, mobile phase gradient: 0 to 100% EtOAc in heptane) yielding Compound C34 (1.57 g, 89%).

A microwave tube was charged with Compound C34 (100 mg, 0.22 mmol), benzyl bromide (reagent b) (76 mg, 0.44 mmol), Cs₂CO₃ (142 mg, 0.44 mmol) and DMF (1 mL). The tube was sealed and the reaction mixture was stirred at 80° C. for 1 h. The volatiles were removed under reduced pressure. The residue was purified via preparative HPLC (Stationary phase: RP XBridge Prep C18 OBD-10 μm, 30×150 mm, Mobile phase: 0.25% NH₄HCO₃ solution in water, CH₃CN) to give Compound C41 (15 mg, 13%).

The reaction mixture was stirred at room temperature overnight in the synthesis of Compound C51.

The reaction mixture was stirred at 110° C. for 16 hours in the synthesis of Compounds C58 and C65 and the solvent of the reaction was DMF.

### Procedure G

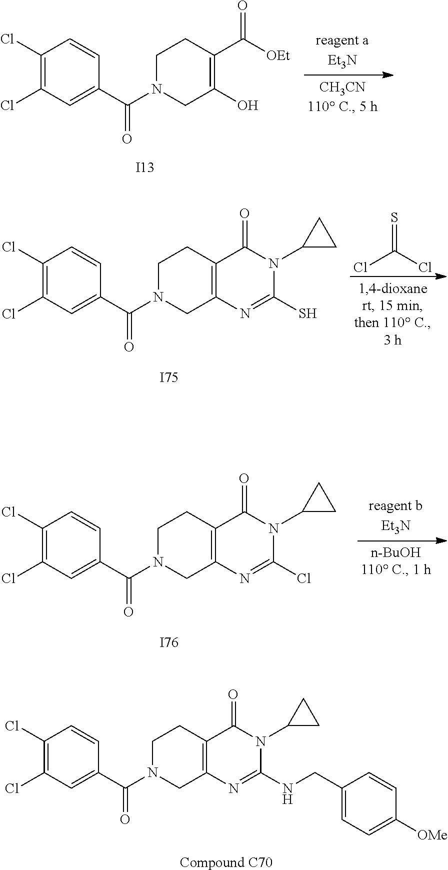

A solution of intermediate I13 (250 mg, 0.73 mmol), N-cyclopropylthiourea (reagent a) (75.9 mg, 0.65 mmol) and Et₃N (0.15 mL, 1.09 mmol) in CH₃CN (5 mL) was stirred at 110° C. for 5 h. The volatiles were removed under reduced pressure and the residue was purified by flash column chromatography (silica, mobile phase gradient: heptane to EtOAc:EtOH:NH₃ (3:1:0.02)) to afford 3-cyclopropyl-7-(3,4-dichloro-benzoyl)-2-sulfanyl-5,6,7,8-tetrahydropyrido[3,4-d]pyrimidin-4(3H)-one (Intermediate I75) (178 mg, 62%).

For the synthesis of Compound C64, DBU was used instead of Et₃N and the reaction mixture was stirred at 80° C. for 5 days.

The reaction mixture was stirred at room temperature overnight in the synthesis of Compound C71.

The reaction mixture was stirred at 110° C. overnight in the synthesis of Compound C72.

Thiophosgene (44.4 μL, 0.56 mmol) was added to a suspension of Intermediate I75 (178 mg, 0.45 mmol) in 1,4-dioxane (3 mL). The reaction mixture was stirred at room temperature for 15 min, and at 110° C. for 3 h. The volatiles were removed under reduced pressure and the residue was used in the next step without further purification.

Crude Intermediate I76, 4-methoxybenzylamine (reagent b) (235 μL, 1.80 mmol) and Et₃N (250 μL, 1.80 mmol) were dissolved in n-BuOH (3 mL) and the reaction mixture was stirred at 110° C. for 1 h. The volatiles were removed under reduced pressure and the residue was purified by preparative HPLC (Stationary phase: RP XBridge Prep C18 OBD-10 μm, 50×150 mm, Mobile phase: 0.25% NH₄HCO₃ solution in water, CH₃CN). A second purification was performed via preparative HPLC (Stationary phase: RP XBridge Prep C18 OBD-10 μm, 30×150 mm, Mobile phase: 0.25% NH₄HCO₃ solution in water, MeOH) to afford Compound C70 (58.5 mg, 26%).

### Procedure H

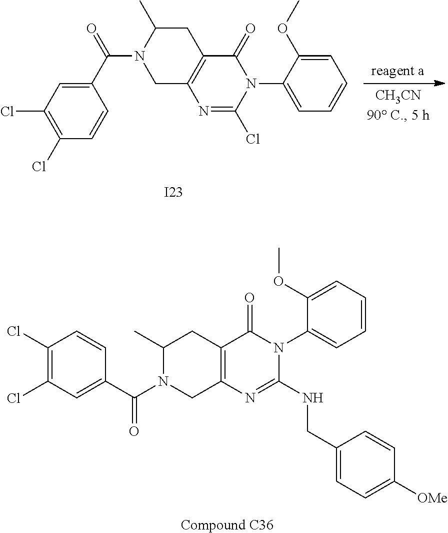

A microwave tube was charged with a solution of Intermediate I23 (250 mg, 0.52 mmol) in dry CH₃CN (5 mL) and 4-methoxybenzylamine (reagent a) (0.68 mL, 5.17 mmol). The tube was sealed and the reaction mixture was stirred at 90° C. for 5 h. The solvents were evaporated under reduced pressure and the residue was purified by flash column chromatography (silica, mobile phase gradient: 0 to 100% EtOAc in DCM) to give Compound C36 (254 mg, 84%) as a beige powder.

The 4 isomers (two enantiomers and their corresponding rotamers) were separated via preparative SFC (Stationary phase: Chiralpak Diacel AD 20×250 mm, Mobile phase: CO₂, EtOH+0.4% i-PrNH₂) yielding Compound C36a (58 mg, 19%), Compound C36b (33 mg, 11%), Compound C36c (30 mg, 9%) and Compound C36d (54 mg, 18%).

For the synthesis of Compound C95, the reaction was performed under neat conditions with 20 equivalents of NH₃. The reaction mixture was stirred at 110° C. for 30 days.

### Procedure I

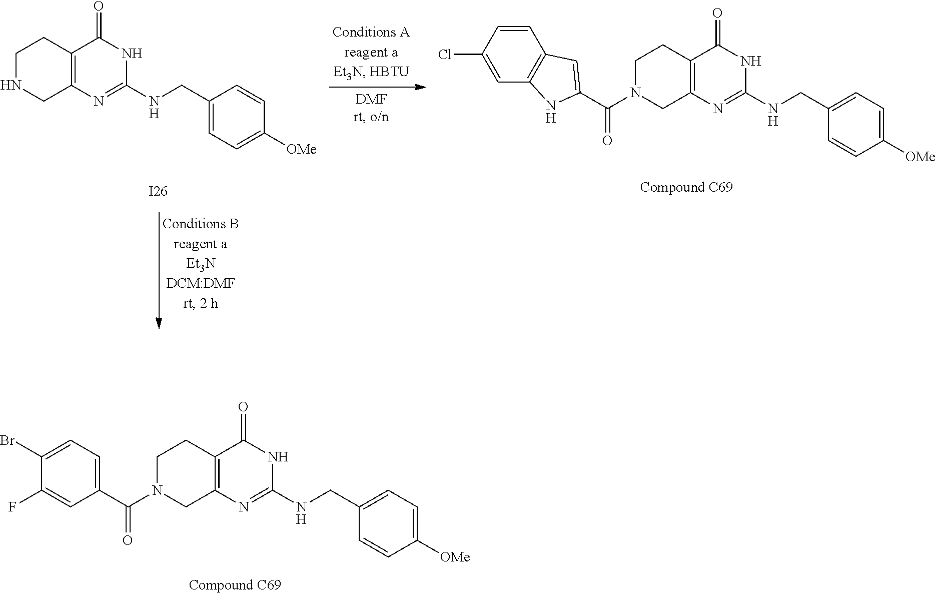 [SMILES: "I26" ; COc1ccc(CNc2nc3c(c(=O)[nH]2)CCNC3)cc1 ; COc1ccc(CNc2nc3c(c(=O)[nH]2)CCN(C(=O)c2cc4ccc(Cl)cc4[nH]2)C3)cc1 ; "Compound C69" ; COc1ccc(CNc2nc3c(c(=O)[nH]2)CCN(C(=O)c2ccc(Br)c(F)c2)C3)cc1 ; "Compound C69"]

### Conditions A

A mixture of Intermediate I26 (255 mg, 496 μmol), 6-chloroindole-2-carboxylic acid (reagent a) (100 mg, 0.51 mmol), Et₃N (0.3 mL, 2.16 mmol) and HBTU (185 mg, 0.49 mmol) in DMF (2.5 ml) was stirred at room temperature overnight The mixture was loaded on a silica cartridge and purified by flash column chromatography (silica, mobile phase gradient: 60 to 100% EtOAc in heptane, then to 25% EtOH in EtOAc). The residue was triturated in CH₃CN (˜5 mL), filtered off and dried at 50° C. under vacuum overnight to afford Compound C79 (26 mg, 11%) as a white powder. Et₃N was replaced by DIPEA in the synthesis of Compound C60.

### Conditions B

A mixture of Intermediate I26 (247 mg, 0.48 mmol), 4-bromo-3-fluorobenzoyl chloride (reagent a) (148 mg, 0.62 mmol) and Et₃N (0.33 mL, 2.40 mmol) in DCM (2 ml-) and DMF (2 ml-) was stirred at room temperature for 2 h. The mixture was loaded on a silica cartridge and purified by flash column chromatography (silica, mobile phase gradient: from 75 to 100% EtOAc in heptane, then from 0 to 25% EtOH in EtOAc). The residue was triturated in CH₃CN (5 ml-) and the white powder was filtered off and dried overnight under vacuum to give Compound C69 (28 mg, 12%).

DCM was not used as co-solvent in the synthesis of Compounds C106, C115, C116, C117 and C118.

### Procedure J

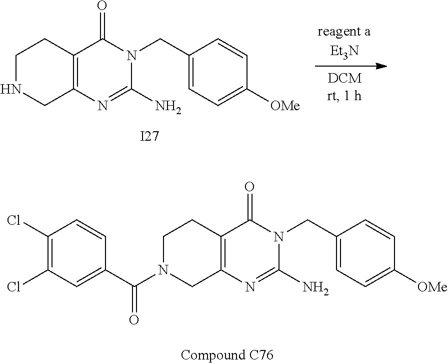 [SMILES: "I27" ; COc1ccc(Cn2c(N)nc3c(c2=O)CCNC3)cc1 ; COc1ccc(Cn2c(N)nc3c(c2=O)CCN(C(=O)c2ccc(Cl)c(Cl)c2)C3)cc1 ; "Compound C76"]

A mixture of Intermediate I27 (3.75 g, 5.97 mmol), 3,4-dichlorobenzoyl chloride (reagent a) (1.31 g, 6.27 mmol) and Et₃N (4.15 mL, 29.8 mmol) in DCM (75 mL) was stirred at room temperature for 30 min. The mixture was loaded on a silica cartridge and purified by flash column chromatography (silica, mobile phase gradient: 50 to 100% EtOAc in heptane, then 0 to 100% EtOAc/EtOH (3:1) in EtOAc) to give Compound C76 (2.06 g, 75%) as a white powder.

### Procedure K

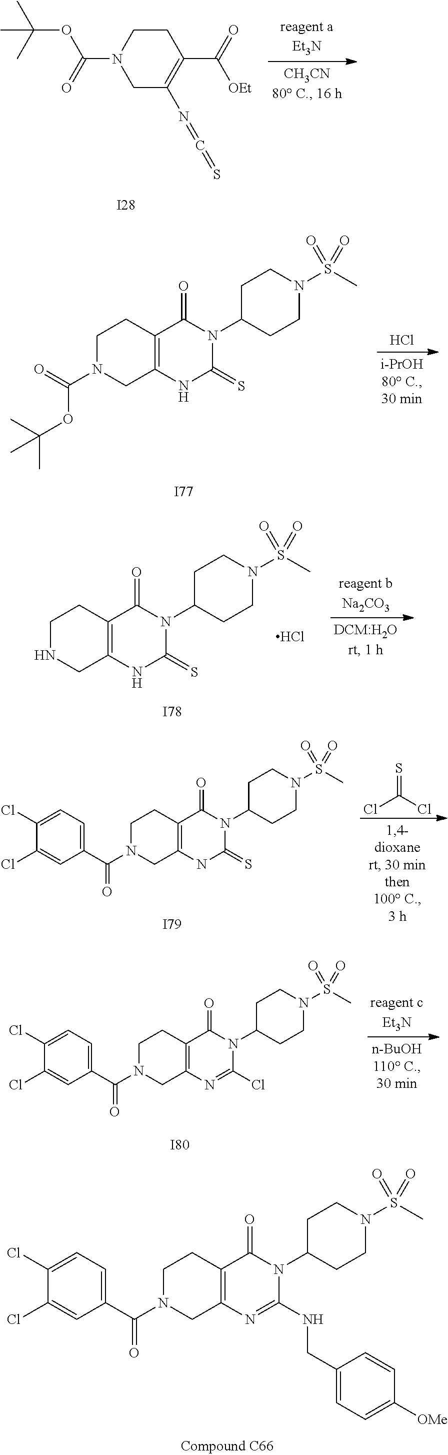

A mixture of Intermediate I28 (500 mg, 1.60 mmol), 4-amino-1-methanesulfonyl-piperidine (reagent a) (371 mg, 2.08 mmol), Et₃N (0.25 mL, 1.76 mmol) and dry CH₃CN (10 mL) in a sealed tube was stirred at 80° C. for 16 h. The reaction mixture was cooled to room temperature and the precipitate was filtered off to afford tert-butyl 3-(1-(methylsulfonyl)piperidin-4-yl)-4-oxo-2-thioxo-2,3,4,5,6,8-hexahydropyrido[3,4-d]pyrimidine-7(1H)-carboxylate (Intermediate I77) (617 mg, 87%) as a white powder.

In a sealed tube, HCl (6M in i-PrOH, 2.83 mL, 17 mmol) was added to a solution of Intermediate I77 (617 mg, 1.39 mmol) in i-PrOH (15 mL). The reaction mixture was stirred at 80° C. for 30 min and then allowed to cool to room temperature. The precipitate was filtered off and washed with DIPE to give 3-(1-(methylsulfonyl)-piperidin-4-yl)-2-thioxo-2,3,5,6,7,8-hexahydropyrido[3,4-d]pyrimidin-4(1H)-one hydrochloride (Intermediate I78) (370 mg, 70%) as a white powder.

Intermediate I78 (370 mg, 0.97 mmol) was dissolved in a mixture of DCM (5 mL) and water (5 mL). 3,4-Dichlorobenzoyl chloride (reagent b) (220 mg, 1.02 mmol) was added followed by Na₂CO₃ (206 mg, 1.94 mmol) portion wise over a period of 10 min. The reaction mixture was stirred at room temperature for 1 h. The precipitate was filtered off, washed with DIPE and dried under vacuum to afford 7-(3,4-dichloro-benzoyl)-3-(1-(methylsulfonyl)piperidin-4-yl)-2-thioxo-2,3,5,6,7,8-hexahydropyrido[3,4-d]pyrimidin-4(1H)-one (Intermediate I79) (180 mg) as a white solid. 2-MeTHF was used as organic solvent instead of DCM in the synthesis of Compound C68.

A microwave vial was charged with Intermediate I79 (180 mg, 0.35 mmol) in dry 1,4-dioxane (3 mL). Thiophosgene (34 μL, 0.44 mmol) was added and the reaction mixture was stirred at room temperature for 30 min and then at 100° C. for 30 min. The reaction mixture was cooled to room temperature and concentrated under reduced pressure to afford 2-chloro-7-(3,4-dichlorobenzoyl)-3-[1-(methanesulfonyl)-piperidin-4-yl]-5,6,7,8-tetrahydropyrido[3,4-d]pyrimidin-4(3H)-one (Intermediate I80).

In a sealed tube, Intermediate I80 was dissolved in n-BuOH (3 mL) and 4-methoxybenzylamine (reagent c) (0.14 mL, 1.05 mmol) and Et₃N (0.097 mL, 0.70 mmol) were added. The reaction mixture was stirred at 110° C. for 30 min and the reaction mixture was cooled to room temperature. The precipitate was filtered and the filtrate was concentrated under reduced pressure. The residue was dissolved in DCM and the precipitate was filtered. The filtrate was loaded on a silica cartridge and purified by flash column chromatography (silica, mobile phase gradient: 0 to 100% EtOAc/EtOH 3:1+0.2% MeOH/NH₃(7N) in heptane). A second purification was performed via preparative HPLC (Stationary phase: RP XBridge Prep C18 OBD-10 μm, 50×150 mm, Mobile phase: 0.25% NH₄HCO₃ solution in water, CH₃CN). The residue was dissolved in MeOH and concentrated under reduced pressure, triturated in DIPE, filtered and dried under vacuum to afford Compound C66 (68 mg, 31%) as a white powder.

### Procedure L

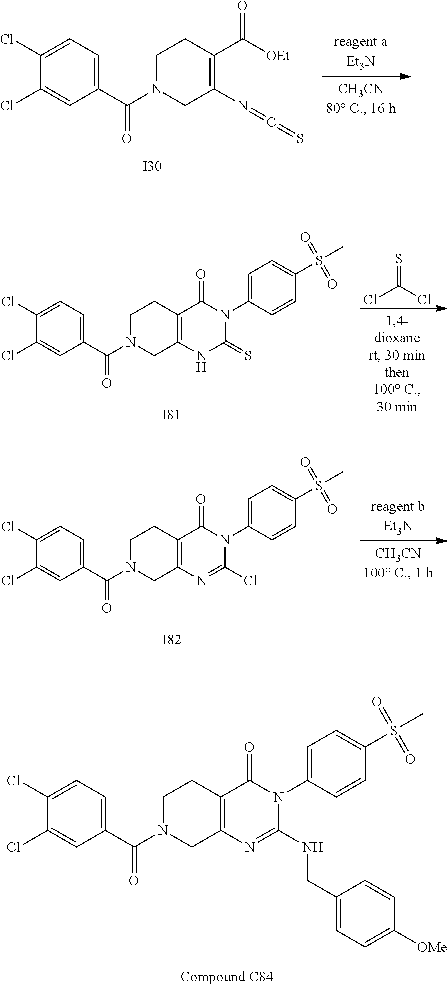

In a pressure tube, a mixture of Intermediate I30 (500 mg, 1.30 mmol), 4-(methyl-sulfonyl)aniline (reagent a) (295 mg, 1.69 mmol), Et₃N (0.2 mL, 1.43 mmol) and dry CH₃CN (7.3 mL) was stirred at 80° C. for 16 h. The mixture was cooled to room temperature and the precipitate was filtered off and dried under vacuum to afford 7-(3,4-dichlorobenzoyl)-3-[4-(methanesulfonyl)phenyl]-2-sulfanylidene-2,3,5,6,7,8-hexahydropyrido[3,4-d]pyrimidin-4(1H)-one (Intermediate I81) (287 mg, 43%).

The reaction mixture was stirred at 60° C. for 16 hours in the synthesis of Compounds C87 and C88.

The reaction mixture was stirred at room temperature for 1 hour in the synthesis of Compounds C111 and C112.

When no precipitate was observed, the reaction mixture was cooled to room temperature and concentrated under reduced pressure. Then, the crude mixture was purified by flash column chromatography.

A microwave vial was charged with Intermediate I81 (275 mg, 0.54 mmol) in dry 1,4-dioxane (4.35 mL). Thiophosgene (47 μL, 0.59 mmol) was added and the reaction mixture was stirred at room temperature for 30 min and at 100° C. for 30 min. The reaction mixture was cooled to room temperature, loaded on a silica cartridge and purified by flash column chromatography (silica, mobile phase gradient: 0 to 100% EtOAc in heptane) to afford 2-chloro-7-(3,4-dichlorobenzoyl)-3-[4-(methanesulfonyl)-phenyl]-5,6,7,8-tetrahydropyrido[3,4-d]pyrimidin-4(3H)-one (Intermediate I82) (200 mg, 72%) as a white solid.

A microwave vial was charged with Intermediate I82 (100 mg, 0.20 mmol), 4-methoxybenzylamine (reagent b) (28 μL, 0.22 mmol), Et₃N (41 μL, 0.29 mmol) and dry CH₃CN (3 mL) was stirred at 100° C. for 1 h. The reaction mixture was cooled to room temperature and concentrated under reduced pressure. The residue was partitioned between water and DCM. The layers were separated and the organic phase was dried (MgSO₄), filtered and concentrated under reduced pressure. The product was crystallized from CH₃CN, filtered off and dried under vacuum to give Compound C84 (54 mg, 45%) as a white solid.

The solvent of the reaction mixture was n-BuOH in the synthesis of Compounds C77, C78, C83, C85, C87, C88, C93 and C120.

The reaction mixture was stirred at 110° C. overnight in the synthesis of Compound C93.

The reaction mixture was stirred at 80° C. for 16 hours in the synthesis of Compounds C96, C99, C102, C108, C111, C125, C127, C131, C143, C144, C148, C156, C160, C162, C210, C219 and C220.

For Compounds C77 and C78, once the reaction was complete, the reaction mixture was cooled to room temperature and the residue was dissolved in DCM. The mixture was filtered and the filtrate was loaded on a silica cartridge. The mixture was purified by flash column chromatography and/or by preparative HPLC. The residue was triturated in DIPE, filtered off and dried under vacuum.

Upon completion of the reaction, the mixture was cooled to room temperature and concentrated under reduced pressure. The residue was partitioned between DCM and water. The organic layer was separated, dried (MgSO₄), filtered and concentrated under reduced pressure. The crude mixture was purified by purified by flash column chromatography and/or by preparative HPLC. The residue was eventually triturated in DIPE, filtered off and dried under vacuum to afford the desired product. This procedure was followed for Compounds C74, C85, C87, C88, C96, C156, C160 and C162.

Upon completion of the reaction, the mixture was cooled to room temperature and concentrated under reduced pressure. The crude mixture was purified by flash column chromatography and/or by preparative HPLC. The residue was eventually triturated in DIPE, filtered off and dried under vacuum to afford the desired product. This procedure was followed for the synthesis of Compounds C83, C93, C99, C102, C108, C111, C112, C119, C120, C121, C126, C131, C143, C144, C210, C219 and C220.

Upon completion of the reaction, the mixture was cooled to room temperature and concentrated under reduced pressure. The residue was triturated in MeOH and stirred for 10 min at room temperature. The precipitate was filtered off, washed with DIPE and dried under vacuum. This procedure was followed for the synthesis of Compounds C125 and C127.

### Procedure M

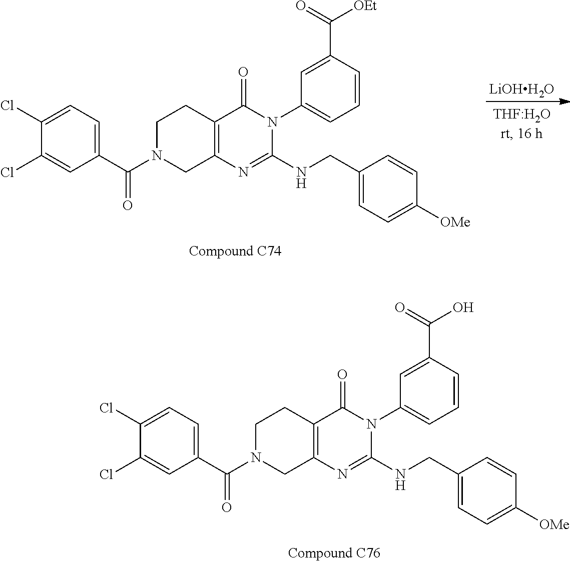 [SMILES: CCOC(=O)c1cccc(-n2c(NCc3ccc(OC)cc3)nc3c(c2=O)CCN(C(=O)c2ccc(Cl)c(Cl)c2)C3)c1 ; "LiOH·H2O" ; COc1ccc(CNc2nc3c(c(=O)n2-c2cccc(C(=O)O)c2)CCN(C(=O)c2ccc(Cl)c(Cl)c2)C3)cc1]

To a solution of Compound C74 (158 mg, 0.26 mmol) in THF (3.3 mL) and H₂O (1.6 mL) was added LiOH.H₂O (21.8 mg, 0.52 mmol). The reaction mixture was stirred at room temperature for 16 h and concentrated under reduced pressure. The residue dissolved in water. HCl (1 M in H₂O, 0.52 mL, 0.52 mmol) was added and the mixture was stirred at room temperature for 1 h. The product was filtered off and washed with CH₃CN and DIPE. The product was dried under vacuum to give Compound C75 (75 mg, 50%) as a white solid.

In the synthesis of Compounds C254 and C280, the mixture was extracted with 2-MeTHF and the organic layer was concentrated under reduced pressure. The residue was triturated in CH₃CN, filtered off and dried under vacuum.

### Procedure N

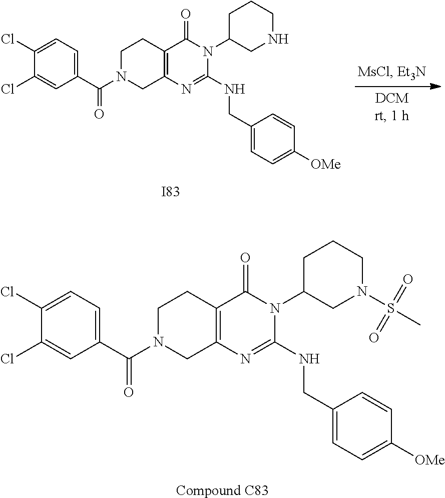

A mixture of 7-(3,4-dichlorobenzoyl)-2-{[(4-methoxyphenyl)methyl]amino}-3-(piperidin-3-yl)-5,6,7,8-tetrahydropyrido[3,4-d]pyrimidin-4(3H)-one (Intermediate I83) (58 mg, 0.11 mmol), methanesulfonyl chloride (9.93 μL, 0.13 mmol) and Et₃N (59.3 μL, 0.43 mmol) in DCM (5 mL) was stirred at room temperature for 1 h. The volatiles were removed under reduced pressure and the residue was purified by flash column chromatography (silica, mobile phase gradient: heptane to EtOAc) to afford Compound C83 (22.8 mg, 34%).

### Procedure O

The reaction sequence was carried out in the presence of ethyl and methyl ester intermediates, but Compound C94 was isolated as a pure product.

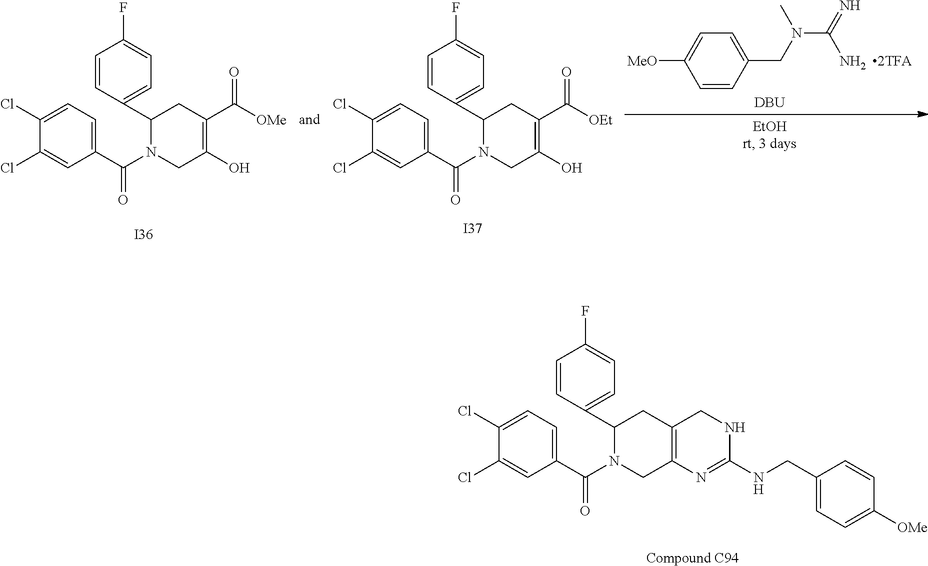

To a solution of Intermediates I36 and I37 (44 mg) in EtOH (0.60 mL) was added N-[(4-methoxyphenyl)methyl]-N-guanidine.2TFA (20.1 mg, 49.4 μmol) followed by DBU (24.6 μL, 0.17 mmol) and the reaction mixture was stirred at room temperature for 3 days. The reaction mixture was concentrated under reduced pressure. The residue was purified via Prep HPLC (Stationary phase: RP XBridge Prep C18 OBD-10 μm, 30×150 mm, Mobile phase: 0.25% NH₄HCO₃ solution in water, CH₃CN). A second purification was performed by flash column chromatography (silica, mobile phase gradient: DCM/MeOH, 100:0 to 95:5) to give Compound C94 (17 mg) as a white solid.

### Procedure P

The reaction sequence was carried out in the presence of methyl and ester intermediates.

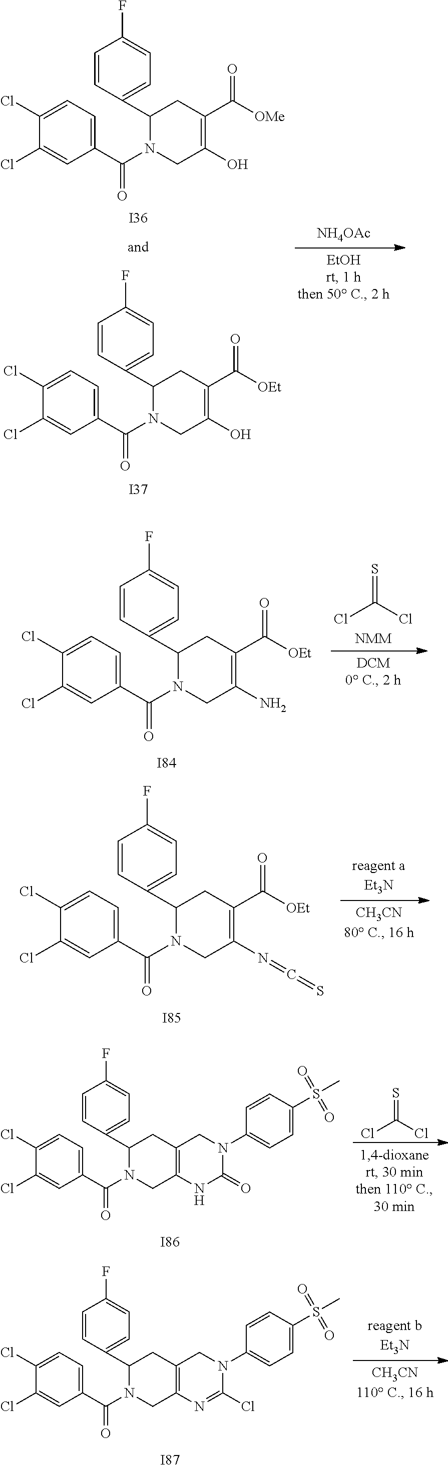

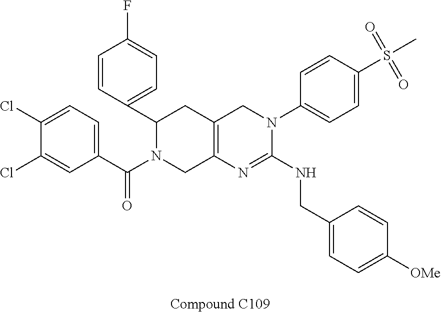 [SMILES: COc1ccc(CNC2=NC3=C(CC(c4ccc(F)cc4)N(C(=O)c4ccc(Cl)c(Cl)c4)C3)CN2c2ccc(S(C)(=O)=O)cc2)cc1 ; "Compound C109"]

A mixture of Intermediates I36 and I37 (1.01 g) and NH₄OAc (0.92 g, 11.9 mmol) were dissolved in EtOH (6.95 mL). The reaction mixture was stirred at room temperature for 1 h and at 50° C. for 2 h. The reaction mixture was cooled to room temperature and concentrated under reduced pressure. The crude mixture was purified by flash column chromatography (silica, mobile phase gradient: DCM/EtOAc, 0:1 to 1:0) to afford a mixture of methyl- and ethyl-5-amino-1-(3,4-dichlorobenzoyl)-2-(4-fluorophenyl)-1,2,3,6-tetrahydropyridine-4-carboxylate (Intermediate I84) (836 mg) as a yellow solid.

A mixture of Intermediate I84 (836 mg) and NMM (0.53 mL, 4.78 mmol) were dissolved in dry DCM (5.0 mL). The mixture was cooled in an ice bath and thiophosgene (0.19 mL 2.39 mmol) was added dropwise at 0° C. The reaction mixture was stirred at 0° C. for 2 h. The mixture was loaded on a silica cartridge and purified by flash column chromatography (mobile phase gradient: heptane/EtOAc, 100:0 to 1:1) to afford a mixture of methyl- and ethyl-1-(3,4-dichlorobenzoyl)-2-(4-fluorophenyl)-5-isothiocyanato-1,2,3,6-tetrahydropyridine-4-carboxylate (Intermediate I85) (834 mg) as a yellow foam.

In a sealed tube, Intermediate I85 (500 mg), 4-(methylsulfonyl)aniline (reagent a) (237 mg, 1.36 mmol), Et₃N (0.16 mL 1.15 mmol) and dry CH₃CN (6.0 mL) was stirred at 80° C. for 16 h. The reaction mixture was cooled to room temperature and concentrated under reduced pressure. The crude mixture was purified by flash column chromatography (silica, mobile phase gradient: heptane/EtOAc, 100:0 to 0:100). The residue was triturated in MeOH, filtered and dried under vacuum to afford (3,4-dichlorophenyl)[6-(4-fluorophenyl)-3-[4-(methanesulfonyl)phenyl]-2-sulfanylidene-2,3,4,5,6,8-hexahydropyrido[3,4-d]pyrimidin-7(1H)-yl]methanone (Intermediate I86) (366 mg) as a white solid.

A microwave tube was charged with Intermediate I86 (366 mg, 605 μmol) and 1,4-dioxane (4.2 mL). The tube was sealed under N₂ atmosphere and thiophosgene (47.8 μL, 605 μmol) was added. The reaction mixture was stirred at room temperature for 30 min and then at 110° C. for another 30 min. The mixture was cooled to room temperature and loaded on a silica cartridge. The crude mixture was purified by flash column chromatography (silica, mobile phase gradient: heptane/EtOAc, 100:0 to 0:100) to afford [2-chloro-6-(4-fluorophenyl)-3-[4-(methanesulfonyl)phenyl]-4,5,6,8-tetrahydropyrido[3,4-d]pyrimidin-7(3H)-yl](3,4-dichlorophenyl)methanone (Intermediate I87) (367 mg, quant.) as a yellow foam.

A microwave tube was charged with a solution of Intermediate I87 (194 mg, 0.32 mmol) in dry CH₃CN (6 mL), 4-methoxybenzylamine (reagent b) (62.6 μL, 479 μmol) and Et₃N (66.7 μL, 479 μmol) under N₂ atmosphere. The microwave tube was sealed and the reaction mixture was stirred at 110° C. for 16 h. The reaction mixture was poured into water (10 mL) and the suspension was stirred at room temperature for 5 h. The white solid was filtered off, washed with DIPE and dried under vacuum for 16 h to give Compound C109 (181 mg, 80%).

The enantiomers were separated by Prep SFC (Stationary phase: Chiralpak Diacel AS 20×250 mm, Mobile phase: CO₂, EtOH+0.4% i-PrNH₂) to afford Compound C109a (64 mg, 29%) and Compound C109b (69 mg, 31%) as off white solids.

The reaction was performed in the absence of Et₃N for the synthesis of Compound C110.

### Procedure Q

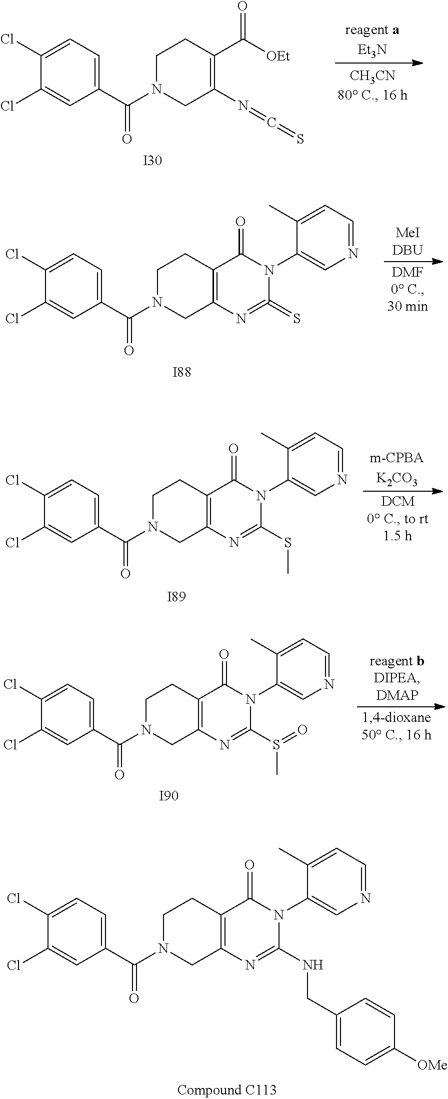

In a sealed tube, a mixture of Intermediate I30 (500 mg, 1.27 mmol), 3-amino-4-methylpyridine (reagent a) (182 mg, 1.65 mmol), Et₃N (0.19 mL, 1.40 mmol) and dry CH₃CN (7.2 mL) was stirred at 80° C. for 16 h. The reaction mixture was cooled to room temperature and concentrated under reduced pressure. The crude mixture was purified by flash column chromatography (silica, mobile phase gradient: 0 to 100% EtOH/EtOAc (1:3) in heptane). The residue was triturated in DIPE, filtered off and dried under vacuum to afford 7-(3,4-dichlorobenzoyl)-3-(4-methylpyridin-3-yl)-2-sulfanylidene-2,3,5,6,7,8-hexahydropyrido[3,4-d]pyrimidin-4(1H)-one (Intermediate I88) (527 mg, 93%) as a yellow solid.

For the synthesis of Compounds C128 and C130, the reaction mixture was cooled to room temperature and the precipitate was filtered off, washed with DIPE and dried under vacuum.

To a solution of Intermediate I88 (527 mg, 1.19 mmol) in dry DMF (4.7 mL) were added DBU (211 μL, 1.41 mmol) and Mel (80.7 μL, 1.30 mmol) at 0° C. The reaction mixture was stirred at 0° C. for 30 min and the reaction was quenched with a saturated aqueous solution of NaHCO₃. The layers were separated and the aqueous phase was extracted with 2-MeTHF. The combined organic extracts were dried (MgSO₄), filtered and concentrated under reduced pressure. The crude mixture was purified by flash column chromatography (silica, mobile phase gradient: 0 to 100% EtOAc in heptane) to afford 7-(3,4-dichlorobenzoyl)-3-(4-methylpyridin-3-yl)-2-(methylsulfanyl)-5,6,7,8-tetrahydropyrido[3,4-d]pyrimidin-4(3H)-one (Intermediate I89) (431 mg, 79%) as a pale yellow foam.

The crude mixture was purified by trituration in CH₃CN. The solid was filtered off and dried under vacuum to afford the desired product. This purification step was applied in the synthesis of Compounds C128 and C130.

A solution of Intermediate I89 (100 mg, 217 μmol) in dry DCM (3 mL) was cooled to O ° C. and m-CPBA (58.3 mg, 0.26 mmol, 77% purity) was added. The reaction mixture was stirred at this temperature for 1 h. K₂CO₃ (136 mg, 0.98 mmol) was added and the mixture was stirred at room temperature for 30 min. The reaction mixture was filtered and concentrated under reduced pressure to afford 7-(3,4-dichlorobenzoyl)-2-(methanesulfinyl)-3-(4-methylpyridin-3-yl)-5,6,7,8-tetrahydropyrido[3,4-d]pyrimidin-4(3H)-one (Intermediate I90) (103 mg, quant.) as a white foam.

A microwave vial was charged with Intermediate I90 (103 mg, 217 μmol), 4-methoxybenzylamine (reagent b) (42.5 μL, 0.33 mmol), DIPEA (48.6 μL, 0.28 mmol) and DMAP (2.37 mg, 19.4 μmol) in dry 1,4-dioxane (2.1 mL). The reaction mixture was stirred at 50° C. for 16 h, cooled to room temperature and concentrated under reduced pressure. The crude mixture was purified by Prep HPLC (Stationary phase: RP XBridge Prep C18 OBD-10 μm, 30×150 mm, Mobile phase: 0.25% NH₄HCO₃ solution in water, CH₃CN). The residue was dissolved in MeOH and concentrated under reduced pressure to afford Compound C113 (43 mg, 36%) as a white solid.

The reaction mixture was stirred at 110° C. for 1 hour for the synthesis of Compound C114.

The reaction was performed in the absence of DIPEA for the synthesis of Compound C134.

For the synthesis of Compound C130, the crude mixture was purified by flash column chromatography (silica, mobile phase gradient: 0 to 100% EtOAc in heptane).

### Procedure R

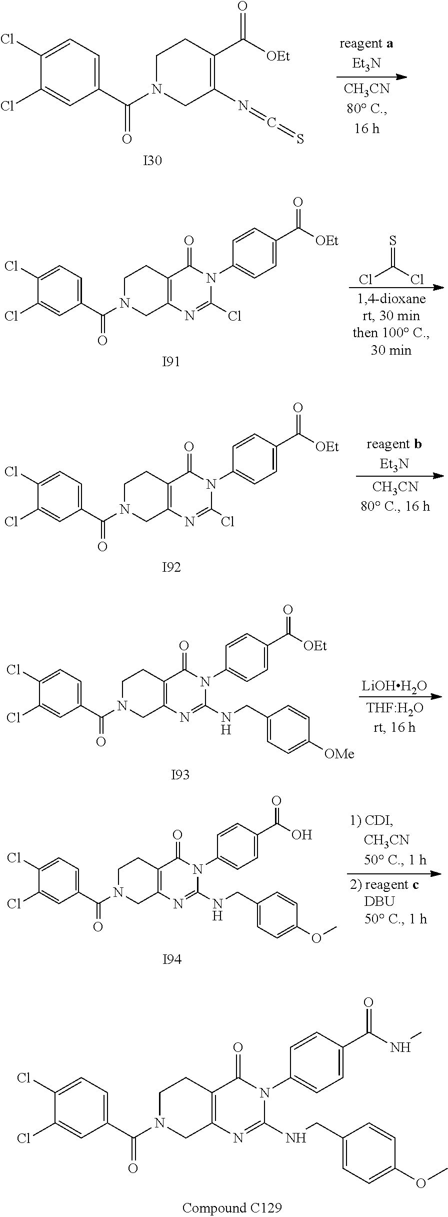

In a pressure tube, a mixture of Intermediate I30 (1.00 g, 2.60 mmol), ethyl 4-aminobenzoate (reagent a) (569 mg, 3.37 mmol) and Et₃N (397 μL, 2.86 mmol) in dry CH₃CN (14.7 mL) was stirred at 80° C. for 16 h. The reaction mixture was cooled to room temperature. The precipitate was filtered off and dried under vacuum to afford ethyl 4-(7-(3,4-dichlorobenzoyl)-4-oxo-2-thioxo-1,4,5,6,7,8-hexahydropyrido[3,4-d]pyrimidin-3(2H)-yl)benzoate (intermediate I91) (984 mg, 75%) as a yellow solid.

In a sealed tube, to a solution of Intermediate I91 (667 mg, 1.32 mmol) in dry 1,4-dioxane (15.4 mL, 180 mmol) was added thiophosgene (115 μL, 1.46 mmol). The reaction mixture was stirred at room temperature for 30 min and at 100° C. for 10 min. The reaction mixture was cooled to room temperature and concentrated under reduced pressure. The crude mixture was purified by flash column chromatography (silica, mobile phase gradient: 0 to 100% EtOAc in heptane) to afford ethyl 4-(2-chloro-7-(3,4-dichlorobenzoyl)-4-oxo-5,6,7,8-tetrahydropyrido[3,4-d]pyrimidin-3(4H)-yl)benzoate (Intermediate I92) (670 mg, quant.) as a white foam.

In a sealed tube, a mixture of Intermediate I92 (670 mg, 1.32 mmol), 4-methoxybenzylamine (reagent b) (207 μL, 1.59 mmol), Et₃N (0.37 mL, 2.64 mmol) and dry CH₃CN (15 mL) was stirred at 80° C. for 16 h. The reaction mixture was cooled to room temperature and concentrated under reduced pressure. The residue was purified by flash column chromatography (silica, mobile phase gradient: 0 to 100% EtOAc in heptane) to afford ethyl 4-(7-(3,4-dichlorobenzoyl)-2-((4-methoxy-benzyl)amino)-4-oxo-5,6,7,8-tetrahydropyrido[3,4-d]pyrimidin-3(4H)-yl)benzoate (Intermediate I93) (691 mg, 86%) as a white foam.

Intermediate I93 (691 mg, 1.14 mmol) was dissolved in THF (14.3 mL) and water (7.1 mL). LiOH.H₂O (95.5 mg, 2.28 mmol) was added and the reaction mixture was stirred at room temperature for 16 h. The reaction mixture was concentrated under reduced pressure. The residue was dissolved in water. HCl (1 M in H₂O, 2.3 mL, 2.3 mmol) was added and the mixture was stirred at room temperature for 10 min. The solid was filtered off and dried under vacuum. The residue was triturated in DIPE, filtered off and dried under vacuum. A purification was performed by flash column chromatography (silica, mobile phase gradient: 0 to 100% EtOAc/EtOH (3:1 with 2% AcOH) in heptane. The residue was triturated in CH₃CN, filtered off and dried under vacuum to afford 4-(7-(3,4-dichlorobenzoyl)-2-((4-methoxybenzyl)amino)-4-oxo-5,6,7,8-tetrahydropyrido[3,4-d]pyrimidin-3(4H)-yl)benzoic acid (Intermediate I94) (466 mg, 71%) as a white solid.

In a sealed tube, a mixture of Intermediate I94 (100 mg, 173 μmol) in CH₃CN (3 mL) and CDI (42.0 mg, 0.26 mmol) was stirred at 50° C. for 1 h. The reaction mixture was cooled to room temperature and methylamine (reagent c) (6.43 mg, 207 μmol) and DBU (50 μL, 0.34 mmol) were added. The reaction mixture was stirred at 50° C. for 1 h. The reaction mixture was cooled to room temperature and concentrated under reduced pressure. The crude mixture was purified by flash column chromatography (silica, mobile phase gradient: 0 to 100% EtOAc/EtOH (3:1) in heptane to give Compound C129 (78 mg, 76%) as a white powder. Another purification was performed via Prep HPLC (Stationary phase: RP XBridge Prep C18 OBD-10 μm, 30×150 mm, Mobile phase: 0.25% NH₄HCO₃ solution in water, CH₃CN). The residue was dissolved in MeOH and concentrated under reduced pressure. The product was triturated in DIPE, filtered and dried under vacuum to give Compound C129 (17 mg, 17%) as a white solid.

### Procedure S

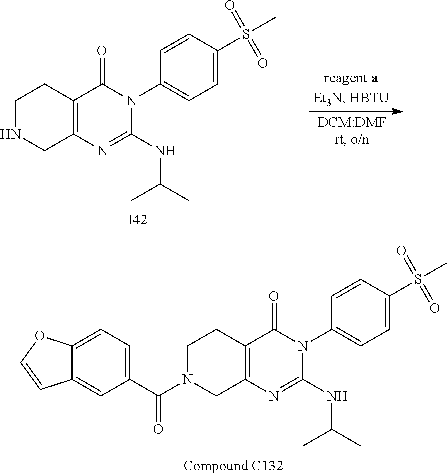 [SMILES: "I42" ; CC(C)Nc1nc2c(c(=O)n1-c1ccc(S(C)(=O)=O)cc1)CCNC2 ; CC(C)Nc1nc2c(c(=O)n1-c1ccc(S(C)(=O)=O)cc1)CCN(C(=O)c1ccc3occc3c1)C2]

A solution of Intermediate I42 hydrochloride (300 mg, 752 μmol) in dry DCM (3 mL) and dry DMF (1.5 mL) was added to a mixture of 1-benzofuran-5-carboxylic acid (reagent a) (142 mg, 876 μmol), dry Et₃N (0.9 mL, 6.48 mmol) and HBTU (340 mg, 897 μmol) in dry DCM (3 mL). The reaction mixture was stirred at room temperature overnight. The mixture was loaded on a silica cartridge and purified by flash column chromatography (silica, mobile phase gradient: 60 to 100% EtOAc in heptane, then 0 to 25% EtOH in EtOAc). The residue was triturated in MeOH (˜25 mL) and filtered to give Compound C132 (210 mg, 55%).

The reaction was performed in the absence of HBTU and DMF for the synthesis of Compounds C152 and C221.

The reaction was performed in the absence of DMF for the synthesis of Compounds C151, C155, C164, C165, C181, C182, C184, C191, C193, C194, C222, C223, C224, C225.

The crude mixture was purified by Prep HPLC (Stationary phase: RP XBridge Prep C18 OBD-10 μm, 30×150 mm, Mobile phase: 0.25% NH₄HCO₃ solution in water, CH₃CN). The product was precipitated in the water phase and filtered off for Compounds C133, C147, C163, C164, and C165.

Compound C137 was crystallized from CH₃CN and filtered off.

### Procedure T

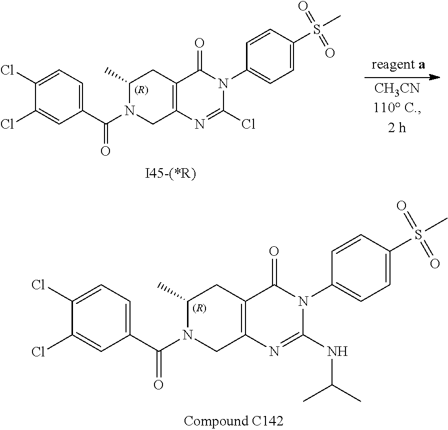

In a microwave tube, to a solution of Intermediate I45-(R) (100 mg, 0.17 mmol, 89% purity) in dry CH₃CN (2.0 mL) was added isopropylamine (reagent a) (0.15 mL, 1.78 mmol) under N₂ atmosphere. The tube was sealed and the reaction mixture was stirred at 110° C. for 2 h. The reaction mixture was cooled to room temperature and the white crystals were filtered off and washed with water and DIPE to give Compound C142 (65 mg).

For Compounds C146, C150 and C198, the reaction was performed in the presence of DIPEA (10 equivalents).

For Compound C207, the reaction was performed in the presence of Et₃N (2 equivalents) at 80° C. for 4 days.

The following work-up was used for Compound C145. The reaction mixture was cooled to room temperature and the mixture was diluted with MeOH. The crystals were filtered off and washed with water and DIPE to afford the desired product.

For Compounds C146, C198 and C207, the crude mixture was purified via Prep HPLC (Stationary phase: RP XBridge Prep C18 OBD-10 μm, 30×150 mm, Mobile phase: 0.25% NH₄HCO₃ solution in water, CH₃CN). The residue was triturated in DIPE and filtered.

For Compound C199, the crude mixture was purified by flash column chromatography (silica, mobile phase gradient: 0 to 10% MeOH in DCM). The residue was crystallized from MeOH, filtered off, washed with DIPE and dried under vacuum.

### Procedure U

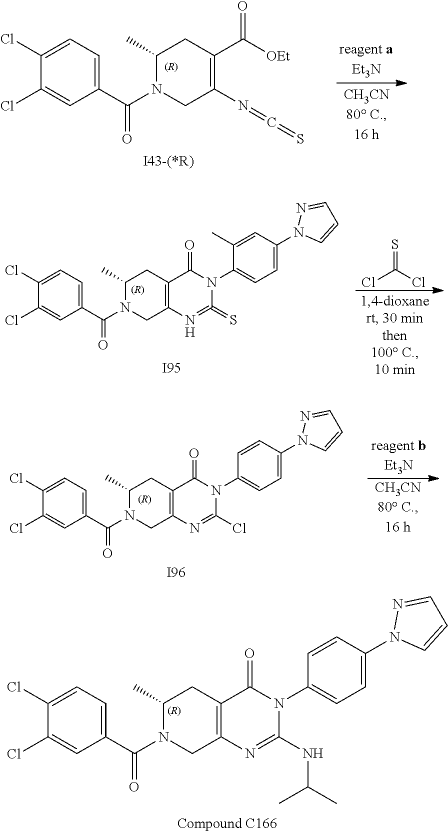

A pressure tube was charged with intermediate I43-(R) (500 mg, 1.24 mmol), 4-(1H-pyrazol-1-yl)aniline (257 mg, 1.61 mmol) and Et₃N (0.26 mL, 1.86 mmol) in dry CH₃CN (7 mL). The reaction mixture was stirred at 80° C. for 16 h. The mixture was cooled to room temperature and the mixture was concentrated under reduced pressure. The crude mixture was purified by flash column chromatography (silica, mobile phase gradient: 0 to 100% EtOAc in heptane). The residue was triturated in DIPE, filtered off and dried under vacuum to afford (6R)-7-(3,4-dichlorobenzoyl)-6-methyl-3-[4-(1H-pyrazol-1-yl)phenyl]-2-sulfanylidene-2,3,5,6,7,8-hexahydropyrido[3,4-d]pyrimidin-4(1H)-one (Intermediate I95) (490 mg, 77%) as a white solid. In the synthesis of Compound C292, DBU was used instead of Et₃N.

The reaction mixture was stirred at 90° C. for 17 days for the synthesis of Compounds C340a and C340b.

For Compound C153, the reaction mixture was cooled to room temperature, and the solid was filtered off and washed with water and DIPE.

A pressure tube was charged with Intermediate I95 (490 mg, 0.96 mmol) in dry 1,4-dioxane (11 mL). Thiophosgene (83.1 μL, 1.05 mmol) was added and the reaction mixture was stirred at room temperature for 30 min, then at 100° C. for 10 min. The mixture was concentrated under reduced pressure. The residue was purified by flash column chromatography (silica, mobile phase gradient: 0 to 100% EtOAc in heptane) to afford (6R)-2-chloro-7-(3,4-dichlorobenzoyl)-6-methyl-3-[4-(1H-pyrazol-1-yl)phenyl]-5,6,7,8-tetrahydropyrido[3,4-d]pyrimidin-4(3H)-one (Intermediate I96) (200 mg, 41%).

The reaction mixture was stirred at room temperature for 30 min and at then at 110° C. for 1 hour for the synthesis of Compounds C153, C183, C226, C227, C228, C233, C234, C236, C279, C281, C293, C300a, C300b, C308, C308a, C308b, C314a, C314b, C340a and C340b.

A microwave vial was charged with Intermediate I96 (200 mg, 0.39 mmol), isopropylamine (67 μL, 0.78 mmol), Et₃N (108 μL, 0.78 mmol) and dry CH₃CN (3.9 mL). The reaction mixture was stirred at 80° C. for 16 h. The mixture was cooled to room temperature and the mixture was concentrated under reduced pressure. The residue was purified by flash column chromatography (silica, mobile phase gradient: 0 to 100% EtOAc in heptane). The residue was triturated in DIPE, filtered off and dried under vacuum to give Compound C166 (115 mg, 55%) as a white solid. DIPEA was used instead of Et₃N in the synthesis of Compound C230.

The reaction mixture was stirred at 110° C. for 2 h in the synthesis of Compounds C153and C183.

The reaction was performed in the absence of Et₃N in the synthesis of Compounds C228, C233, C234, C236, C279, C281, C287, C292, C293, C300a, C300b, C308, C308a, C308b, C314a, C314b, C331, C335, C340a, and C340b.

For Compounds C183 and C323, the mixture was partially evaporated under reduced pressure, and the white solid was filtered off and washed with water and DIPE.

For the following compounds the work up was modified: Compounds C206, C229, C230, C254, C273, C274, C318 and C322. The reaction mixture was cooled to room temperature. The mixture was concentrated under reduced pressure and purified following the procedure described below, or a workup was done prior to the purification. Work-up: The mixture was cooled to room temperature and the mixture was concentrated under reduced pressure. The residue was partitioned between water and 2-MeTHF. The layers were separated and the organic phase was dried (MgSO₄), filtered and concentrated under reduced pressure. Purification: The purification was done by flash column chromatography (silica) and/or by preparative HPLC (Stationary phase: RP XBridge Prep C18 OBD-10 μm, 30×150 mm, Mobile phase: 0.25% NH₄HCO₃ solution in water, CH₃CN). The residue was finally triturated in DIPE, filtered and dried under vacuum. Also, when possible, the residue was crystallized from CH₃CN, filtered off and dried under vacuum.

For Compound C267, the reaction mixture was cooled to room temperature and the precipitate was filtered off, washed with DIPE and dried under vacuum.

Compound C287 was obtained after a second purification via a preparative SFC.

### Procedure V

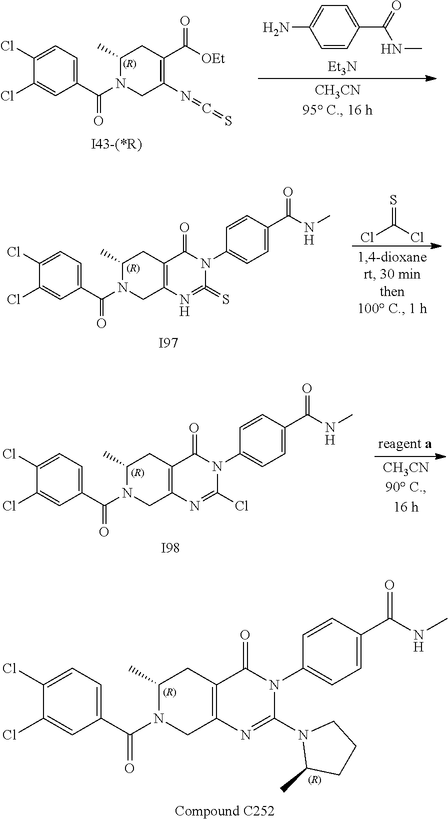

A pressure tube was charged with intermediate I43-(R) (5.00 g, 12.5 mmol), 4-amino-N-methylbenzamide (2.44 g, 16.3 mmol) and Et₃N (4.3 mL, 31.3 mmol) in dry CH₃CN (71 mL). The reaction mixture was stirred at 95° C. for 16 h. The solvents were removed under reduced pressure and the crude mixture was dissolved in DCM (50 mL). On standing, the product crystalized and was filtered off and washed with DCM to afford 4-[(6R)-7-(3,4-dichlorobenzoyl)-6-methyl-4-oxo-2-sulfanylidene-1,4,5,6,7,8-hexahydropyrido[3,4-d]pyrimidin-3(2H)-yl]-N-methylbenzamide (Intermediate I97) (5.02 g, 79%).

In a pressure tube, Intermediate I97 (5.83 g, 11.58 mmol) was suspended in dry 1,4-dioxane (64 mL) and thiophosgene (1.24 mL, 16.2 mmol) was added. The reaction vessel was sealed under N₂ atmosphere and the reaction mixture was stirred at room temperature for 30 min, then at 110° C. for 1 h. The mixture was cooled to room temperature and the solid was filtered off, washed with 1,4-dioxane and DIPE. The product was dried under vacuum at 40° C. for 16 h to afford 4-[(6R)-2-chloro-7-(3,4-dichlorobenzoyl)-6-methyl-4-oxo-5,6,7,8-tetrahydropyrido[3,4-d]pyrimidin-3(4H)-yl]-N-methylbenzamide (Intermediate I98) (4.3 g, 67%) as a yellow powder. The filtrate was charged on a silica cartridge and the mixture was purified by flash column chromatography (silica, mobile phase gradient: 0 to 100% EtOAc in heptane) to afford a second crop of Intermediate I98 (1.45 g, 24%) as a white solid.

To a suspension of Intermediate I98 (150 mg, 0.27 mmol) in dry CH₃CN (1.5 mL) was added (R)-2-methylpyrrolidine (reagent a) (0.27 mL, 2.67 mmol) in a pressure tube. The tube was sealed under N₂ atmosphere and the reaction mixture was stirred at 90° C. for 16 h. The solvents were evaporated under reduced pressure. The crude mixture was purified by flash column chromatography (silica, mobile phase gradient: DCM/MeOH, 100:0 to 90:10). The residue was crystalized in CH₃CN and DIPE and filtered off to give Compound C252 (85 mg, 56%) as a white solid.

DIPEA (2 to 5 equivalents) was added in the reaction mixture for the synthesis of Compounds C251, C277, C278, C282, C283, C284, C302, C303, C306, C311, C312, C316, C320, C321, C333, C334, C336, C337, C342, C343, C347, C348, C349, C350, C351, C352 and C353.

When the reaction was not complete after 16 hours, the reaction mixture was stirred for a longer period of time (sometimes several days).

For Compounds C258, C276, C282, C283, C303, C330, C332, C334 and C337, the reaction mixture was cooled to room temperature and crystallization occurred. The solids were filtered off, washed with water and dried under vacuum.

### Procedure W

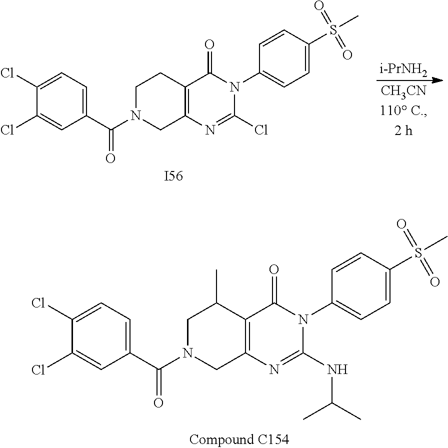 [SMILES: "i-PrNH2" ; "I56" ; CS(=O)(=O)c1ccc(-n2c(Cl)nc3c(c2=O)CCN(C(=O)c2ccc(Cl)c(Cl)c2)C3)cc1 ; CC(C)Nc1nc2c(c(=O)n1-c1ccc(S(C)(=O)=O)cc1)C(C)CN(C(=O)c1ccc(Cl)c(Cl)c1)C2]

In a sealed tube, to a solution of intermediate I56 (108 mg, 168 μmol, 82% purity) in dry CH₃CN (1 mL) was added isopropylamine (76.0 μL, 885 μmol). The reaction mixture was stirred at 110° C. for 2 h and cooled to room temperature. The white solids were filtered off and washed with water and DIPE to give Compound C154 (39 mg, 42%).

The enantiomers were separated via Prep SFC (Stationary phase: Chiralcel Diacel OJ 20×250 mm, Mobile phase: CO₂, EtOH+0.4% i-PrNH₂) to give (*R)-7-(3,4-dichlorobenzoyl)-2-(isopropylamino)-5-methyl-3-(4-(methylsulfonyl)phenyl)-5,6,7,8-tetrahydropyrido[3,4-d]pyrimidin-4(3H)-one (Compound C154a) (30 mg, 33%) and (*S)-7-(3,4-dichlorobenzoyl)-2-(isopropylamino)-5-methyl-3-(4-(methylsulfonyl)-phenyl)-5,6,7,8-tetrahydropyrido[3,4-d]pyrimidin-4(3H)-one (Compound C154b) (25 mg, 27%) both as white solids.

### Procedure X

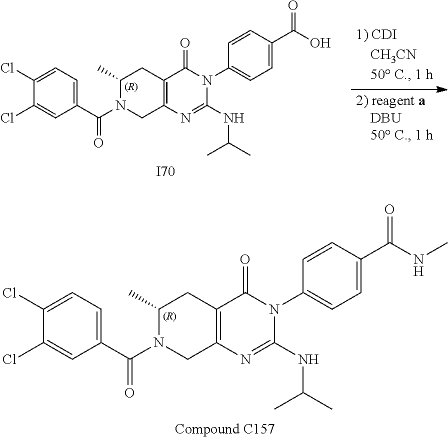

A microwave tube was charged with intermediate I70 (300 mg, 582 μmol) in CH₃CN (10 mL). CDI (142 mg, 873 μmol) was added and the reaction mixture was stirred at 50° C. for 1 h. The reaction mixture was cooled to room temperature and methylamine (reagent a) (21.7 mg, 698 μmol) and DBU (174 μL, 1.16 mmol) were added. The reaction mixture was stirred at 50° C. for 1 h, cooled to room temperature and concentrated under reduced pressure. The crude mixture was purified by flash column chromatography (silica, mobile phase gradient: 0 to 10% MeOH in DCM) to give Compound C157 (196 mg, 64%) as a white solid.

The product was eventually triturated in DIPE, filtered off and dried under vacuum. Compounds C178, C214 and C266 were purified by Prep SFC (Stationary phase: Chiralpak Diacel AD 20×250 mm, Mobile phase: CO₂, i-PrOH+0.4% i-PrNH₂).

### Procedure Y

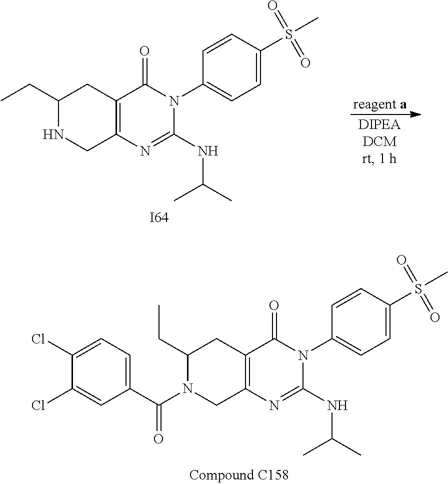 [SMILES: "I64" ; CCC1Cc2c(nc(NC(C)C)n(-c3ccc(S(C)(=O)=O)cc3)c2=O)CN1 ; CCC1Cc2c(nc(NC(C)C)n(-c3ccc(S(C)(=O)=O)cc3)c2=O)CN1C(=O)c1ccc(Cl)c(Cl)c1]

3,4-Dichlorobenzoyl chloride (reagent a) (124 mg, 0.59 mmol) was added to a mixture of intermediate I64 and DIPEA (464 μL 2.69 mmol) in DCM (50 mL). The reaction mixture was stirred at room temperature for 1 h. The reaction mixture was concentrated under reduced pressure and the crude mixture was purified by flash column chromatography (silica, mobile phase gradient: 30 to 100% EtOAc in heptane, then 0 to 25% EtOH in EtOAc). The product was dried under vacuum at 50° C. overnight to give Compound C158 (176 mg, 76% over 2 steps) as a white powder. The enantiomers were separated via Prep SFC (Stationary phase: Chiralcel Diacel OJ 20×250 mm, Mobile phase: CO₂, EtOH+0.4% i-PrNH₂) to afford Compound C158a (65 mg) and Compound C158b (56 mg).

### Procedure Z

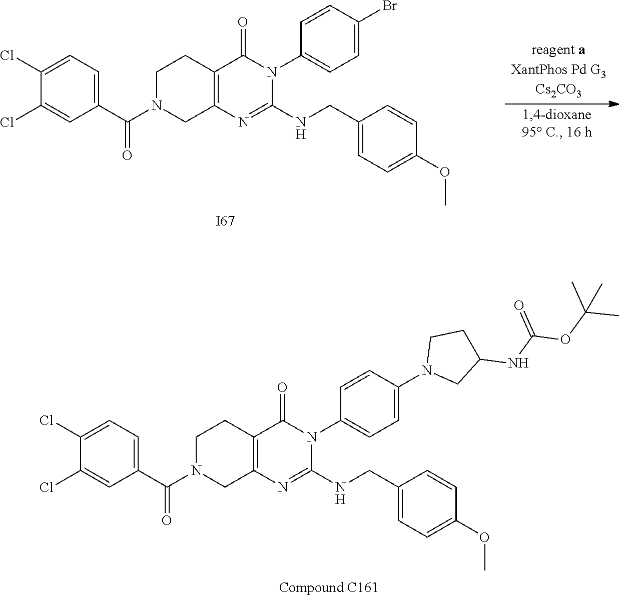 [SMILES: "I67" ; COc1ccc(CNc2nc3c(c(=O)n2-c2ccc(Br)cc2)CCN(C(=O)c2ccc(Cl)c(Cl)c2)C3)cc1 ; COc1ccc(CNc2nc3c(c(=O)n2-c2ccc(N4CCC(NC(=O)OC(C)(C)C)C4)cc2)CCN(C(=O)c2ccc(Cl)c(Cl)c2)C3)cc1]

A microwave vial was charged with Intermediate I67 (200 mg, 326 μmol), 3-(tert-butoxycarbonylamino)pyrrolidine (reagent a) (72.8 mg, 0.39 mmol), Cs₂CO₃ (212 mg, 0.65 mmol) and 1,4-dioxane (6 mL). The mixture was purged with N₂ for 5 min. XantPhos Pd G3 (30.9 mg, 32.6 μmol) was added and the vial was sealed. The reaction mixture was stirred at 95° C. for 16 h, cooled to room temperature and concentrated under reduced pressure. The crude mixture was purified by flash column chromatography (silica, mobile phase gradient: 0 to 100% EtOAc/EtOH (2:1) in heptane). The product was crystallized from CH₃CN, filtered and dried under vacuum to give Compound C161 (90 mg, 38%) as a white solid.

### Procedure AA

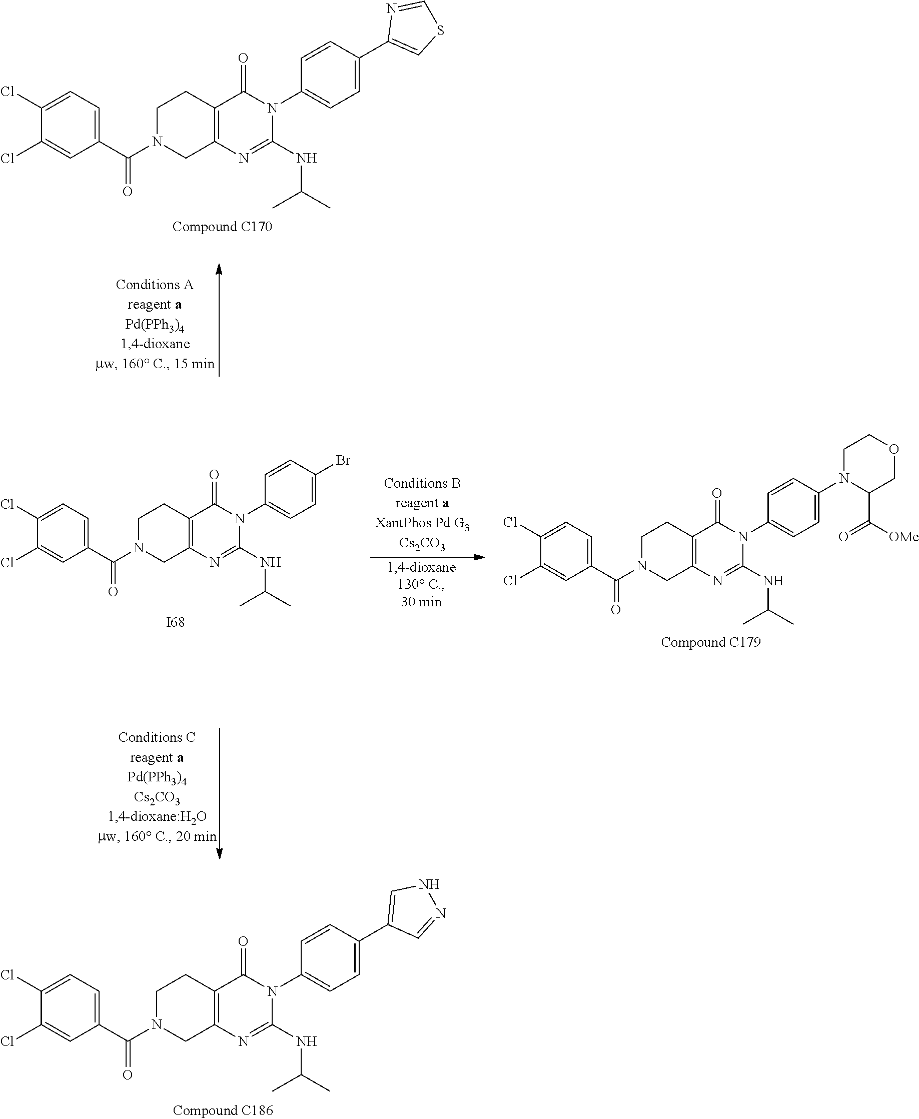 [SMILES: CC(C)Nc1nc2c(c(=O)n1-c1ccc(-c3cscn3)cc1)CCN(C(=O)c1ccc(Cl)c(Cl)c1)C2 ; "I68" ; CC(C)Nc1nc2c(c(=O)n1-c1ccc(Br)cc1)CCN(C(=O)c1ccc(Cl)c(Cl)c1)C2 ; COC(=O)C1COCCN1c1ccc(-n2c(NC(C)C)nc3c(c2=O)CCN(C(=O)c2ccc(Cl)c(Cl)c2)C3)cc1 ; CC(C)Nc1nc2c(c(=O)n1-c1ccc(-c3cn[nH]c3)cc1)CCN(C(=O)c1ccc(Cl)c(Cl)c1)C2]

### Conditions A

In a microwave vial, Intermediate I68 (25.0 mg, 47.0 μmol), 4-(tributylstannyl)thiazole (reagent a) (36.3 mg, 93.0 μmol) and Pd(PPh₃)₄ (5.50 mg, 4.7 μmol) were dissolved in 1,4-dioxane (3 mL). The reaction mixture was heated at 160° C. for 15 min in a microwave. The reaction mixture was diluted with MeOH (30 mL) and filtered. The crude mixture was purified via preparative HPLC (Stationary phase: RP XBridge Prep C18 OBD-10 μm, 50×150 mm, Mobile phase: 0.25% NH₄HCO₃ solution in water, CH₃CN) to afford Compound C170 (97.6 mg, 65%).

For the synthesis of Compound C171, the reaction mixture was microwaved at 160° C. for 1 h.

### Conditions B

A microwave vial was charged with Intermediate I68 (250 mg, 466 μmol), methyl-3-morpholinecarboxylate (reagent a) (81.2 mg, 559 μmol), Cs₂CO₃ (304 mg, 0.93 mmol) and 1,4-dioxane (5 mL). The mixture was purged with N₂ for 5 min. N-XantPhos Pd G3 (44.2 mg, 46.6 μmol) was added and the vial was sealed. The reaction mixture was stirred at 130° C. for 30 min. The reaction mixture was concentrated under reduced pressure. The crude mixture was purified by flash column chromatography (silica, mobile phase gradient: 0 to 100% EtOAc in heptane). A second purification was performed via Prep HPLC (Stationary phase: RP XBridge Prep C18 OBD-10 μm, 30×150 mm, Mobile phase: 0.25% NH₄HCO₃ solution in water, CH₃CN). The residue was dissolved in MeOH and the mixture was evaporated under reduced pressure. The product was triturated in DIPE, filtered and dried under vacuum to afford Compound C179 (73 mg, 26%) as a pale yellow solid.

The work-up for the synthesis of Compound C185 was modified: the mixture was cooled to room temperature and concentrated under reduced pressure. The residue was partitioned between water and DCM. The layers were separated and the organic phase was dried (MgSO₄), filtered and concentrated under reduced pressure. The crude mixture was purified via Prep HPLC (Stationary phase: RP XBridge Prep C18 OBD-10 μm, 30×150 mm, Mobile phase: 0.25% NH₄HCO₃ solution in water, CH₃CN). The residue was dissolved in MeOH and concentrated under reduced pressure. The product was triturated in CH₃CN, filtered off and dried under vacuum.

### Conditions C

Intermediate I68 (200 mg, 0.37 mmol), 1-boc-pyrazole-4-boronic acid pinacol ester (reagent a) (132 mg, 0.45 mmol), Cs₂CO₃ (365 mg, 1.12 mmol) and Pd(PPh₃)₄ (44.0 mg, 37.0 μmol) were dissolved in 1,4-dioxane (3 mL) and water (0.2 mL). The reaction mixture was heated at 160° C. for 20 min in a microwave. The reaction mixture was filtered and purified via preparative HPLC (Stationary phase: RP XBridge Prep C18 OBD-10 μm, 50×150 mm, Mobile phase: 0.25% NH₄HCO₃ solution in water, CH₃CN) to afford Compound C186 (57.1 mg, 29%).

### Procedure AB

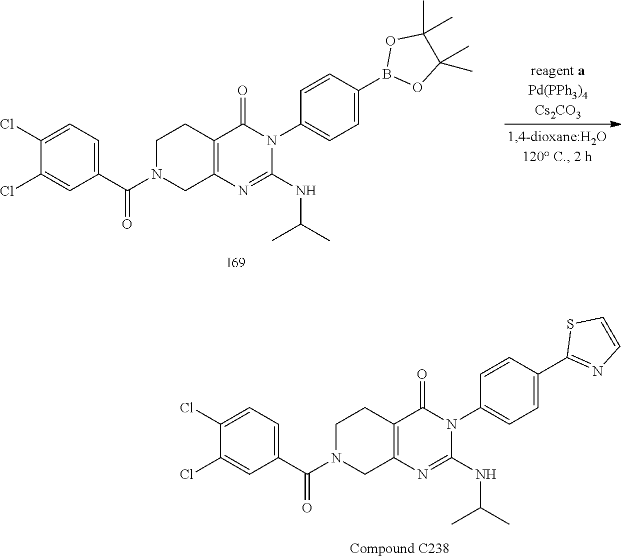 [SMILES: "I69" ; CC(C)Nc1nc2c(c(=O)n1-c1ccc(B3OC(C)(C)C(C)(C)O3)cc1)CCN(C(=O)c1ccc(Cl)c(Cl)c1)C2 ; CC(C)Nc1nc2c(c(=O)n1-c1ccc(-c3nccs3)cc1)CCN(C(=O)c1ccc(Cl)c(Cl)c1)C2]

A mixture of Intermediate I69 (150 mg, 0.26 mmol), 2-bromothiazole (reagent a) (63.3 mg, 0.39 mmol), Cs₂CO₃ (251 mg, 0.77 mmol) and Pd(PPh₃)₄ (30.3 mg, 25.7 μmol) in 1,4-dioxane (3 mL) and water (0.2 mL) was stirred at 120° C. for 2 h. The reaction mixture was cooled to room temperature, diluted with MeOH and filtered. The filtrate was purified via preparative HPLC (Stationary phase: RP XBridge Prep C18 OBD-10 μm, 50×150 mm, Mobile phase: 0.25% NH₄HCO₃ solution in water, CH₃CN) to afford Compound C238 (71.8 mg, 52%).

### Procedure AC

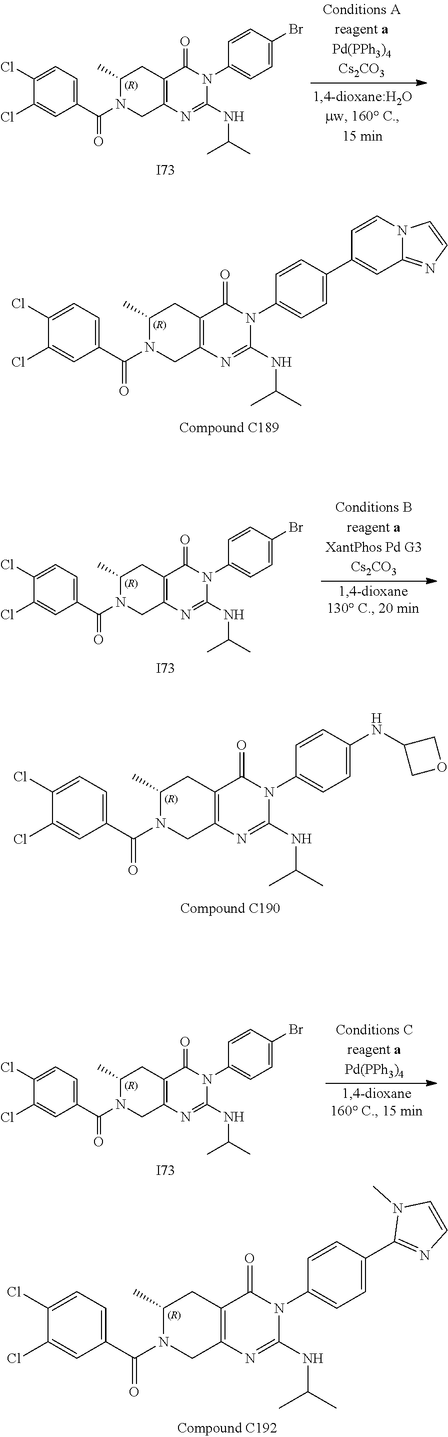

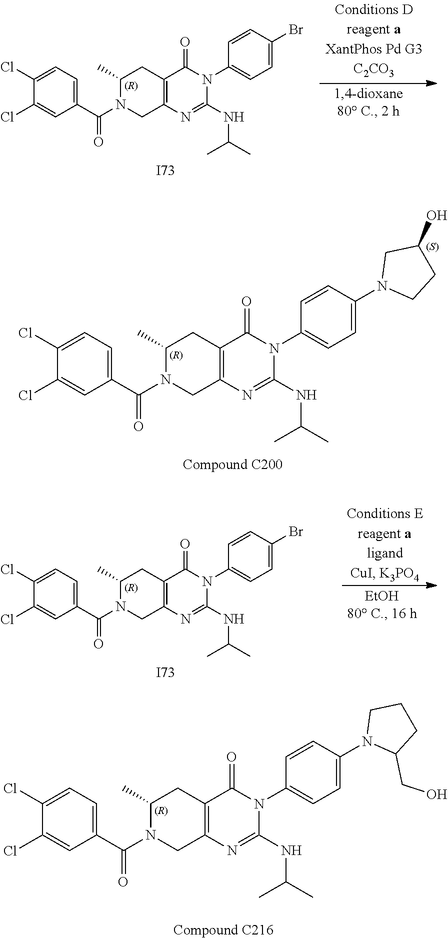

### Conditions A

Intermediate I73 (250 mg, 0.45 mmol), 7-(4,4,5,5-tetramethyl-1,3,2-dioxaborolan-2-yl)imidazo[1,2-a]pyridine (reagent a) (222 mg, 0.91 mmol), Cs₂CO₃ (444 mg, 1.36 mmol) and Pd(PPh₃)₄ (53.6 mg, 45 μmol) were dissolved in 1,4-dioxane (3 mL) and water (0.2 mL). The reaction mixture was heated at 160° C. for 15 min in a microwave. The reaction mixture was cooled to room temperature and filtered. The filtrate was purified via preparative HPLC (Stationary phase: RP XBridge Prep C18 OBD-10 μm, 50×150 mm, Mobile phase: 0.25% NH₄HCO₃ solution in water, CH₃CN) to afford Compound C189 (45.9 mg, 17%).

### Conditions B

A microwave vial was charged with Intermediate I73 (200 mg, 0.36 mmol), 3-oxetamine (reagent a) (31.9 mg, 436 μmol), Cs₂CO₃ (237 mg, 0.73 mmol) and 1,4-dioxane (3 mL). The mixture was purged with N₂ for 5 min. XantPhos Pd G3 (34.5 mg, 36.3 μmol) was added and the vial was sealed. The reaction mixture was stirred at 130° C. for 20 min. The mixture was cooled to room temperature and concentrated under reduced pressure. The residue was partitioned between water and DCM. The layers were separated and the organic layer was dried (MgSO₄), filtered and concentrated under reduced pressure. The crude mixture was purified via Prep HPLC (Stationary phase: RP XBridge Prep C18 OBD-10 μm, 30×150 mm, Mobile phase: 0.25% NH₄HCO₃ solution in water, CH₃CN). The residue was dissolved in MeOH and concentrated under reduced pressure. The product was triturated in CH₃CN, filtered and dried under vacuum to give Compound C190 (28 mg, 14%, 96% purity) as a white solid.

### Conditions C

In a microwave vial, Intermediate I73 (119 mg, 208 μmol) was dissolved in degassed 1,4-dioxane (3 mL). Pd(PPh₃)₄ (24.5 mg, 20.8 μmol) and 1-methyl-2-(tributylstannyl)-1H-imidazole (reagent a) (154 μL, 415 μmol) were added and the tube was sealed under N₂ atmosphere. The reaction mixture was heated at 160° C. for 15 min. The reaction was diluted with MeCN and filtered. A purification was performed via Prep HPLC (Stationary phase: RP XBridge Prep C18 OBD-10 μm, 30×150 mm, Mobile phase: 0.25% NH₄HCO₃ solution in water, CH₃CN) to give Compound C192 (33 mg, 29%) as a white solid.

### Conditions D

A microwave vial was charged with Intermediate I73 (200 mg, 363 μmol), (S)-3-hydroxypyrrolidine (reagent a) (34.8 mg, 0.40 mmol), Cs₂CO₃ (237 mg, 727 μmol) and 1,4-dioxane (3 mL). The mixture was purged with N₂ for 5 min. Xantphos Pd G3 (34.5 mg, 36.3 μmol) was added and the vial was sealed. The reaction mixture was stirred at 80° C. for 2 h. The mixture was concentrated under reduced pressure and the residue was partitioned between water and DCM. The layers were separated and the organic phase was dried (MgSO₄), filtered and concentrated under reduced pressure. The crude mixture was purified by flash column chromatography (silica, mobile phase gradient: 0 to 100% EtOAc in heptane). The residue was triturated in CH₃CN, filtered off and dried under vacuum to give Compound C200 (130 mg, 64%) as a yellow solid. A second purification was performed via Prep HPLC (Stationary phase: RP XBridge Prep C18 OBD-10 μm, 30×150 mm, Mobile phase: 0.25% NH₄HCO₃ solution in water, CH₃CN). The residue was dissolved in MeOH and concentrated in vacuo. The product was triturated in DIPE, filtered off and dried under vacuum to afford Compound C200 (35 mg, 17%) as a white solid.

The work up for Compound C202 was different: The residue was partitioned between water and 2-MeTHF. The layers were separated and the organic phase was dried (MgSO₄), filtered and concentrated

### Conditions E

A microwave vial was charged with Intermediate I73 (100 mg, 182 μmol), pyrrolidine-2-ylmethanol (reagent a) (27.6 mg, 273 μmol), K₃PO₄ (78.7 mg, 0.36 mmol), CuI (3.46 mg, 18.2 μmol), 4,7-dimethoxy-1,10-phenanthroline (ligand) (4.50 mg, 18.2 μmol) in EtOH (2 mL). The reaction mixture was stirred at 80° C. for 16 h. The reaction mixture was cooled to room temperature and concentrated under reduced pressure. The residue was diluted with DCM and filtered over CeliteR and washed with DCM. The filtrate was concentrated under reduced pressure. The crude mixture was purified via Prep HPLC (Stationary phase: RP XBridge Prep C18 OBD-10 μm, 30×150 mm, Mobile phase: 0.25% NH₄HCO₃ solution in water, CH₃CN). The residue was dissolved in MeOH and concentrated under reduced pressure. The product was triturated in DIPE, filtered and dried under vacuum to give Compound C215 (16 mg, 15%) as a white solid.

### Procedure AD

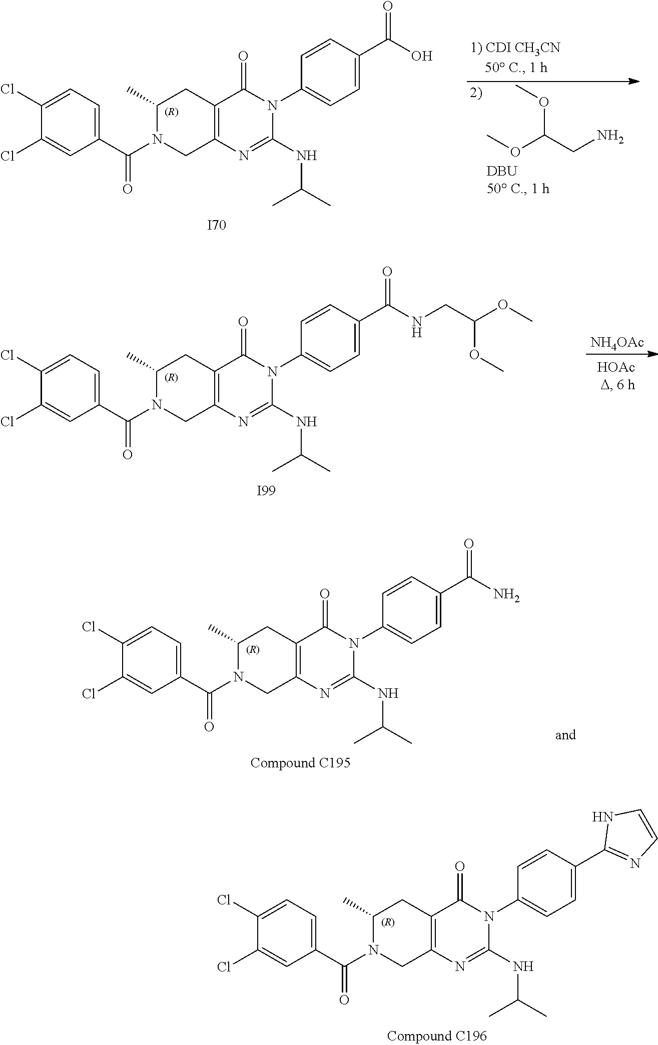

A microwave tube was charged with a mixture of Intermediate I70 (100 mg, 194 μmol) and CDI (47.2 mg, 0.29 mmol) in dry CH₃CN (3.3 mL). The tube was sealed and the reaction mixture was stirred at 50° C. for 1 h. Aminoacetaldehyde dimethyl acetal (25.4 μL 233 μmol) and DBU (58.0 μL, 0.39 mmol) were added and the reaction mixture was stirred at 50° C. for 1 h. The reaction mixture was cooled to room temperature and concentrated under reduced pressure. The crude mixture was purified by flash column chromatography (silica, mobile phase gradient: 0 to 10% MeOH in DCM) to afford (R)-4-(7-(3,4-dichlorobenzoyl)-2-(isopropylamino)-6-methyl-4-oxo-5,6,7,8-tetrahydropyrido[3,4-d]pyrimidin-3(4H)-yl)-N-(2,2-dimethoxyethyl)benzamide (Intermediate I99) (108 mg, 92%) as a white solid.

A mixture of Intermediate I99 (54.0 mg, 89.6 μmol), NH₄OAc (41.5 mg, 538 μmol) and acetic acid (1.5 mL, 26.2 mmol) was stirred under reflux for 6 h. The reaction mixture was cooled to room temperature and concentrated under reduced pressure. The crude mixture was purified via Prep HPLC (Stationary phase: RP XBridge Prep C18 OBD-10 μm, 30×150 mm, Mobile phase: 0.25% NH₄HCO₃ solution in water, CH₃CN) to give Compound C195 (7 mg, 15%) and Compound C196 (7 mg, 14%) as slightly brown solids.

### Procedure AE

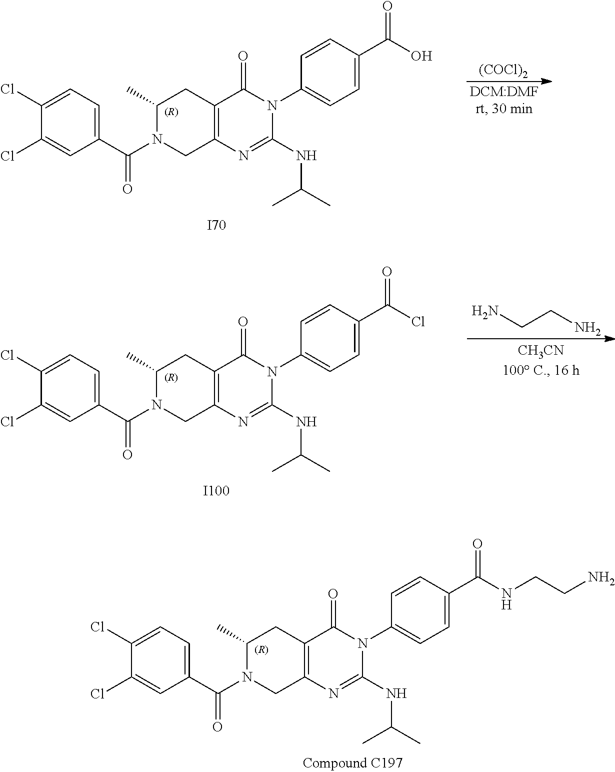

To a solution of Intermediate I70 (131 mg, 254 μmol) in dry DCM (1.63 mL) was added oxalyl chloride (44.3 μmol, 051 mmol) followed by 2 drops of DMF. The reaction mixture was stirred at room temperature for 30 min and concentrated under reduced pressure. The residue was dissolved in CH₃CN (6 mL) and filtered. The filtrate was used as such in the next step.

To a crude solution of (*R)-4-(7-(3,4-dichlorobenzoyl)-2-(isopropylamino)-6-methyl-4-oxo-5,6,7,8-tetrahydropyrido[3,4-d]pyrimidin-3(4H)-yl)benzoyl chloride (Intermediate I100) in CH₃CN was added ethylenediamine (17.0 μL, 254 μmol). The reaction mixture was stirred at 100° C. for 16 h. The reaction mixture was cooled to room temperature and concentrated under reduced pressure. The crude mixture was purified by flash column chromatography (silica, mobile phase gradient: 1 to 15% (NH₃ in MeOH) in DCM. The residue was triturated in DIPE and the suspension was sonicated for 15 min. The powder was filtered and dried under vacuum overnight to give Compound C197 (42 mg, 28%).

### Procedure AF

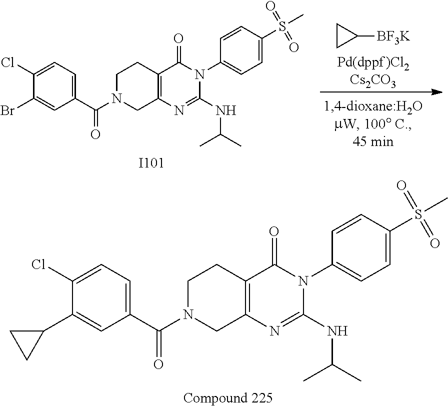

A mixture of 7-(3-bromo-4-chlorobenzoyl)-3-[4-(methanesulfonyl)phenyl]-2-[(propan-2-yl)amino]-5,6,7,8-tetrahydropyrido[3,4-d]pyrimidin-4(3H)-one (Intermediate I101) (201 mg, 347 μmol), potassium cyclopropyltrifluoroborate (86 mg, 0.58 mmol), Pd(dppf)Cl₂ (34 mg, 46.5 μmol) and Cs₂CO₃ (352 mg, 1.08 mmol) in water (0.8 mL) and 1,4-dioxane (10 mL) was heated at 100° C. for 45 min in a microwave tube. The reaction was quenched with water (20 mL). The layers were separated and the aqueous phase was extracted with EtOAc (2×10 mL). The combined organic extracts were dried (MgSO₄), filtered and concentrated under reduced pressure. The crude mixture was purified by flash column chromatography (silica, mobile phase gradient: 0 to 100% EtOAc in heptane). The residue (60 mg) was purified via Prep SFC (Stationary phase: Chiralcel Diacel OJ 20×250 mm, Mobile phase: CO₂, EtOH+0.4% i-PrNH₂). The product was re-crystallized in EtOH to afford Compound C225 (36 mg, 20%) as a white powder.

### Procedure AG

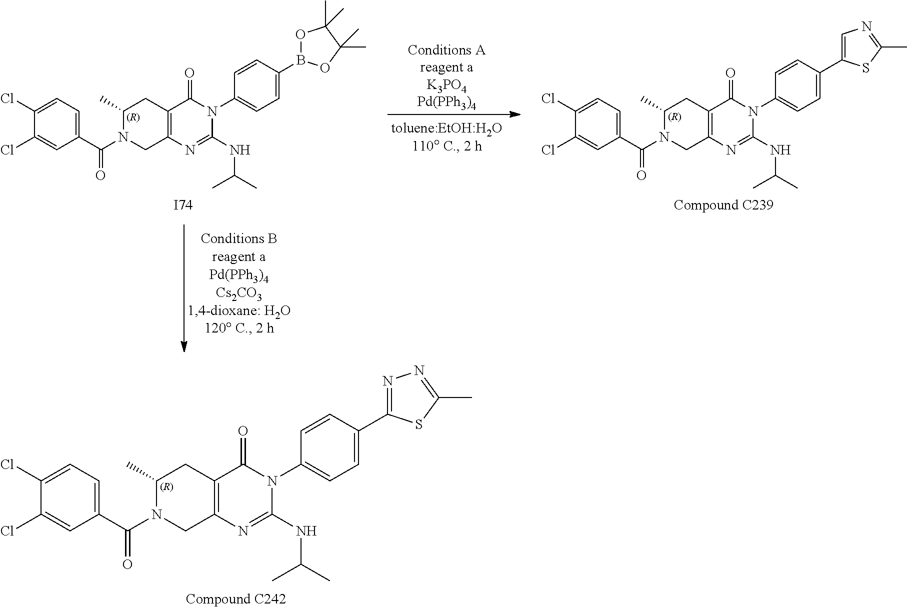

### Conditions A

A microwave vial was charged with Intermediate I74 (218 mg, 365 μmol), 3,5-dimethylisoxazole-4-boronic acid (reagent a) (56.6 mg, 0.40 mmol), K₃PO₄ (158 mg, 0.73 mmol), toluene (820 μL), EtOH (208 mL), and water (365 μL). The mixture was purged with N₂ for 5 min and Pd(PPh₃)₄ (21.5 mg, 18.2 μmol) was added. The vial was sealed and the reaction mixture was stirred at 110° C. for 2 h. The reaction mixture was cooled to room temperature and concentrated under reduced pressure. The crude mixture was purified via Prep HPLC (Stationary phase: RP XBridge Prep C18 OBD-10 μm, 30×150 mm, Mobile phase: 0.25% NH₄HCO₃ solution in water, CH₃CN). The residue was dissolved in MeOH and concentrated under reduced pressure. The product was triturated in DIPE, filtered off and dried under vacuum to afford Compound C239 (48 mg, 23%) as a pale yellow solid.

### Conditions B

Intermediate I74 (200 mg, 0.33 mmol), 2-bromo-5-methyl-1,3,4-oxadiazole (reagent a) (81.8 mg, 0.50 mmol), Cs₂CO₃ (327 mg, 1.00 mmol) and Pd(PPh₃)₄ (39.5 mg, 33.0 μmol) were dissolved in 1,4-dioxane (3 mL) and water (0.5 mL). The reaction mixture was stirred at 120° C. for 2 h. The reaction mixture was cooled to room temperature, diluted with MeOH and filtered. The filtrate was purified via preparative HPLC (Stationary phase: RP XBridge Prep C18 OBD-10 μm, 50×150 mm, Mobile phase: 0.25% NH₄HCO3 solution in water, CH₃CN) to afford Compound C242 (68.3 mg, 37%).

For the synthesis of Compound C247, the work-up was modified: The reaction mixture was cooled to room temperature and diluted with water and 2-MeTHF. The layers were separated and the organic phase was dried over a Isolute HM-N cartridge. The filtrate was evaporated to dryness under reduced pressure and purified via preparative SFC (Stationary phase: Chiralcel Diacel OJ 20×250 mm, Mobile phase: CO₂, EtOH+0.4% i-PrNH₂).

### Procedure AH

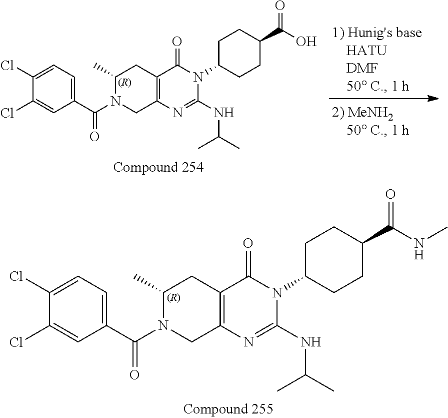

A sealed tube was charged with a solution of Compound C254 (200 mg, 384 μmol) in CH₃CN (6.70 mL), Hunig's base (90.2 μL, 0.52 mmol) and HATU (93.3 mg, 0.58 mmol). The reaction mixture was stirred at 50° C. for 1 h. The reaction mixture was cooled to room temperature and methylamine (14.3 mg, 0.46 mmol) was added. The reaction mixture was stirred at 50° C. for another hour, cooled to room temperature and concentrated under reduced pressure. The crude mixture was purified by flash column chromatography (silica, mobile phase gradient: 0 to 100% EtOAc/EtOH (3:1) in heptane). A second purification was performed via Prep HPLC (Stationary phase: RP XBridge Prep C18 OBD-10 μm, 30×150 mm, Mobile phase: 0.25% NH₄HCO₃ solution in water, CH₃CN). The residue was dissolved in MeOH and concentrated under reduced pressure. The product was triturated in DIPE, filtered off and dried under vacuum to afford Compound C255 (66 mg, 31%) as a white solid.

### Procedure Al

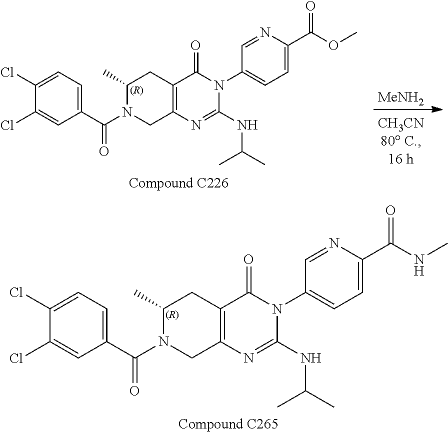

In a sealed tube, to a solution of Compound C226 (189 mg, 0.34 mmol) in dry CH₃CN (3 mL) was added methylamine (105 mg, 3.39 mmol). The tube was sealed and the reaction mixture was stirred at 80° C. for 16 h. The reaction mixture was cooled to room temperature and a white precipitate was observed. The solid was filtered off, washed with water and DIPE to afford Compound C265 (176 mg, 97%) as a white powder.

### Procedure AJ

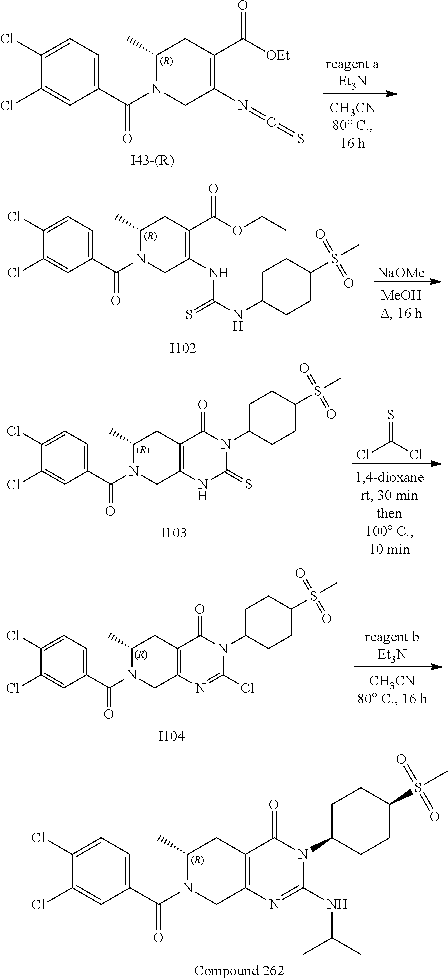

To a mixture of 4-methanesulfonylcyclohexan-1-amine (reagent a) (458 mg, 2.14 mmol) in dry CH₃CN (10.1 mL) was added Et₃N (0.6 mL, 4.46 mmol). The reaction mixture was stirred at room temperature for 5 min. Intermediate I43-(R) (0.72 g, 1.79 mmol) was added and the reaction mixture was stirred for 10 min and at 80° C. for 16h. The reaction mixture was cooled to room temperature and concentrated under reduced pressure. The residue was partitioned between water and 2-MeTHF. The layers were separated and the organic phase was dried (MgSO₄), filtered and concentrated under reduced pressure. The crude mixture was purified by flash column chromatography (silica, mobile phase gradient: 0 to 100% EtOAc/EtOH (2:1) in heptane) to afford ethyl (2R)-1-(3,4-dichlorobenzoyl)-5-({[4-(methanesulfonyl)-cyclohexyl]carbamothioyl}amino)-2-methyl-1,2,3,6-tetrahydropyridine-4-carboxylate (Intermediate I102) (679 mg, 66%).

In a sealed tube, a mixture of Intermediate I102 (679 mg, 1.18 mmol) and NaOMe (63.6 mg, 1.18 mmol) in dry MeOH (5 mL) was stirred under reflux for 16 h. The reaction mixture was cooled to room temperature and concentrated under reduced pressure. The crude mixture was purified by flash column chromatography (silica, mobile phase gradient: 0 to 100% EtOAc/EtOH (2:1) in heptane) to afford (6R)-7-(3,4-dichlorobenzoyl)-3-[4-(methanesulfonyl)cyclohexyl]-6-methyl-2-sulfanylidene-2,3,5,6,7,8-hexahydropyrido[3,4-d]pyrimidin-4(1H)-one (Intermediate I103) (230 mg, 37%).

A sealed tube was charged with Intermediate I103 (460 mg, 0.87 mmol) in dry 1,4-dioxane (10.1 mL). Thiophosgene (75.4 μL, 0.95 mmol) was added and the reaction mixture was stirred at room temperature for 30 min and at 100° C. for 10 min. The reaction mixture was cooled to room temperature and concentrated under reduced pressure. The residue was purified by flash column chromatography (silica, mobile phase gradient: 0 to 100% EtOAc in heptane) to afford (6R)-2-chloro-7-(3,4-dichlorobenzoyl)-3-[4-(methanesulfonyl)cyclohexyl]-6-methyl-5,6,7,8-tetrahydropyrido[3,4-d]pyrimidin-4(3H)-one (Intermediate I104) (355 mg, 77%).

A microwave vial was charged with Intermediate I104 (150 mg, 0.28 mmol), isopropylamine (reagent b) (29.1 μL, 0.34 mmol), Et₃N (78.3 μL, 0.56 mmol) and dry CH₃CN (1.59 mL). The reaction mixture was stirred at 80° C. for 2 h. The reaction mixture was cooled to room temperature and concentrated under reduced pressure. The crude mixture was purified via Prep HPLC (Stationary phase: RP XBridge Prep C18 OBD-10 μm, 30×150 mm, Mobile phase: 0.25% NH₄HCO₃ solution in water, MeOH). The residue was dissolved in MeOH and concentrated under reduced pressure. The product was crystallized from CH₃CN, filtered off and dried under vacuum to afford Compound C262 (60 mg, 38%) as a white solid.

### Procedure AK

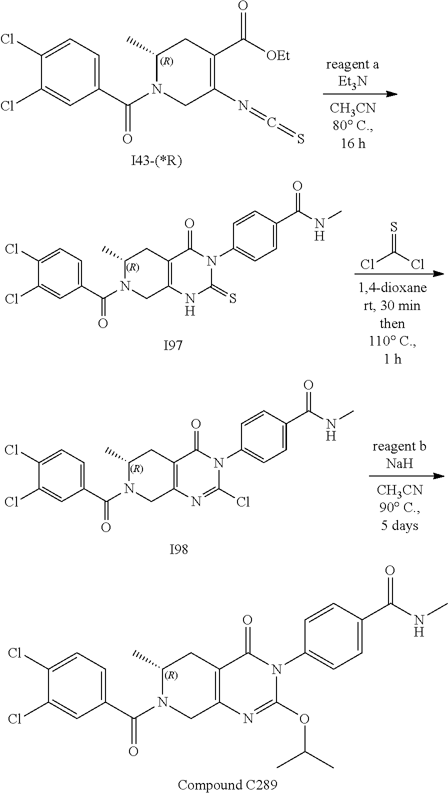

A pressure tube was charged with Intermediate I43-(R) (5.00 g, 12.5 mmol), 4-amino-N-methylbenzamide (reagent a) (2.44 g, 16.3 mmol), Et₃N (4.35 mL, 31.3 mmol) and dry CH₃CN (71 mL). The reaction mixture was stirred at 95° C. for 16 h. The reaction mixture was cooled to room temperature and the solvents were removed under reduced pressure. The residue was dissolved in DCM (50 mL) and left to crystalize. The white crystals were filtered off and washed with little DCM to afford 4-[(6R)-7-(3,4-dichlorobenzoyl)-6-methyl-4-oxo-2-sulfanylidene-1,4,5,6,7,8-hexahydropyrido[3,4-d]pyrimidin-3(2H)-yl]-N-methylbenzamide (Intermediate I97) (5.02 g, 79%). The filtrate was evaporated under reduced pressure, mixed with decalite and the mixture was charged on a column chromatography. The mixture was purified by flash column chromatography (silica, mobile phase: 2.5% MeOH in DCM) to afford a second crop of Intermediate I97 (0.81 g, 12%) as a yellow solid.

In a pressure tube, Intermediate I97 (5.83 g, 11.6 mmol) was suspended in dry 1,4-dioxane (64 mL) and thiophosgene (1.24 mL, 16.2 mmol) was added. The tube was sealed under N₂ atmosphere and the reaction mixture was stirred at room temperature for 30 min, then at 110° C. for 1 h. The reaction mixture was cooled to room temperature. The precipitated was filtered off and washed with little dioxane and DIPE. The residue was dried at 40° C. under vacuum for 16 h to afford a first crop of 4-[(6R)-2-chloro-7-(3,4-dichlorobenzoyl)-6-methyl-4-oxo-5,6,7,8-tetrahydro-pyrido[3,4-d]pyrimidin-3(4H)-yl]-N-methylbenzamide (4.35 g, 67%, 90% purity) as a yellow powder. The filtrate was charged on a silica cartridge and purified by flash column chromatography (silica, mobile phase gradient: 0 to 100% EtOAc in heptane) to afford a second fraction of 4-[(6R)-2-chloro-7-(3,4-dichlorobenzoyl)-6-methyl-4-oxo-5,6,7,8-tetrahydropyrido[3,4-d]pyrimidin-3(4H)-yl]-N-methylbenzamide (Intermediate I98) (1.45 g, 24%) as a white solid.

In a microwave vial, to a solution of Intermediate I98 (150 mg, 267 μmol, 90% purity) in dry CH₃CN (1.40 mL) was added i-PrOH (reagent b) (1.5 mL, 19.6 mmol) followed by NaH (60% dispersion in mineral oil, 16.0 mg, 0.40 mmol). The vial was sealed under N₂ atmosphere and the reaction mixture was stirred at 90° C. for 5 days. Volatiles were evaporated under reduced pressure and the crude mixture was purified by flash column chromatography (silica, mobile phase gradient: 0 to 100% EtOAc in heptane). The residue was crystalized from DIPE and CH₃CN. The product was filtered off and dried under vacuum to afford Compound C289 (45 mg, 32%) as white fluffy solid.

### Procedure AL

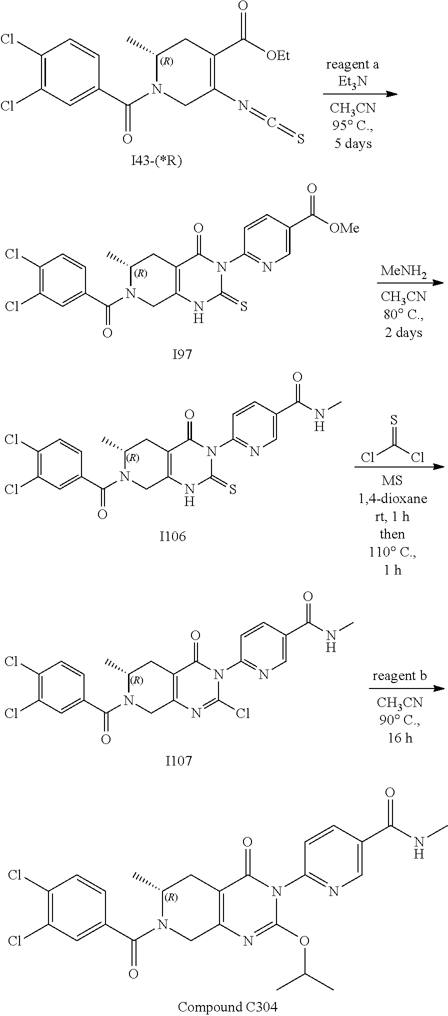

A large μ-wave tube was charged with Intermediate I43-(R) (1.00 g, 2.50 mmol), methyl 6-aminonicotinate (reagent a) (495 mg, 3.26 mmol) and Et₃N (0.87 mL, 6.26 mmol) in dry CH₃CN (14.2 mL). The reaction mixture was stirred at 95° C. for 5 days. The reaction mixture was loaded on a silica cartridge and purified by flash column chromatography (silica, mobile phase gradient: 0 to 10% MeOH in DCM) to afford methyl 6-[(6R)-7-(3,4-dichlorobenzoyl)-6-methyl-4-oxo-2-sulfanylidene-1,4,5,6,7,8-hexahydropyrido[3,4-d]pyrimidin-3(2H)-yl]pyridine-3-carboxylate (Intermediate I105) (463 mg, 34%, 92% purity) as a brown solid.

To a solution of Intermediate I105 (440 mg, 0.871 mmol) in dry CH₃CN (10 mL) in a microwave tube under N₂ atmosphere was added methylamine (270 mg, 8.71 mmol). The tube was sealed and the reaction mixture was stirred at 80° C. for 2 days The mixture was cooled to room temperature and the mixture was charged on a silica cartridge. The mixture was purified by flash column chromatography (silica, mobile phase gradient: 0 to 10% MeOH in DCM). A second purification by flash column chromatography (silica, mobile phase gradient: EtOAc/DCM, 80:20, then DCM/MeOH, 93:7) delivered 6-[(6R)-7-(3,4-dichlorobenzoyl)-6-methyl-2-(methylamino)-4-oxo-5,6,7,8-tetrahydropyrido[3,4-d]pyrimidin-3(4H)-yl]-N-methylpyridine-3-carboxamide (Intermediate I106) (0.27 g, 61%) as a white solid.

Intermediate I106 (0.27 g, 0.53 mmol) was suspended in dry 1,4-dioxane (2.94 mL). Thiophosgene (56.9 μL, 0.74 mmol) and molecular sieves were added. The reaction vessel was sealed under N₂ atmosphere. The reaction mixture was stirred at room temperature for 1 h, and at 110° C. for 1 h. The mixture was cooled to room temperature and loaded on a silica cartridge. The mixture was purified by flash column chromatography (silica, mobile phase gradient: heptane/EtOAc, 100:0 to 0:100) to afford 6-[(6R)-2-chloro-7-(3,4-dichlorobenzoyl)-6-methyl-4-oxo-5,6,7,8-tetrahydropyrido[3,4-d]pyrimidin-3(4H)-yl]-N-methylpyridine-3-carboxamide (Intermediate I107) (214 mg, 64%, 80% purity) as a yellow foam.

To a solution of Intermediate I107 (214 mg, 0.338 mmol, 80% purity) in dry CH₃CN (1.90 mL) in a microwave tube was added isopropylamine (reagent b) (0.29 mL, 3.38 mmol). The tube was sealed under N₂ atmosphere and the reaction mixture was stirred at 90° C. for 16 h. The mixture was charged onto a silica cartridge and purified by flash column chromatography (silica, mobile phase gradient: from 0 to 10% MeOH in DCM). The residue was triturated in CH₃CN and DIPE, and the solid was filtered off to afford Compound C304 (137 mg, 77%) as a yellow solid.

### Procedure AM

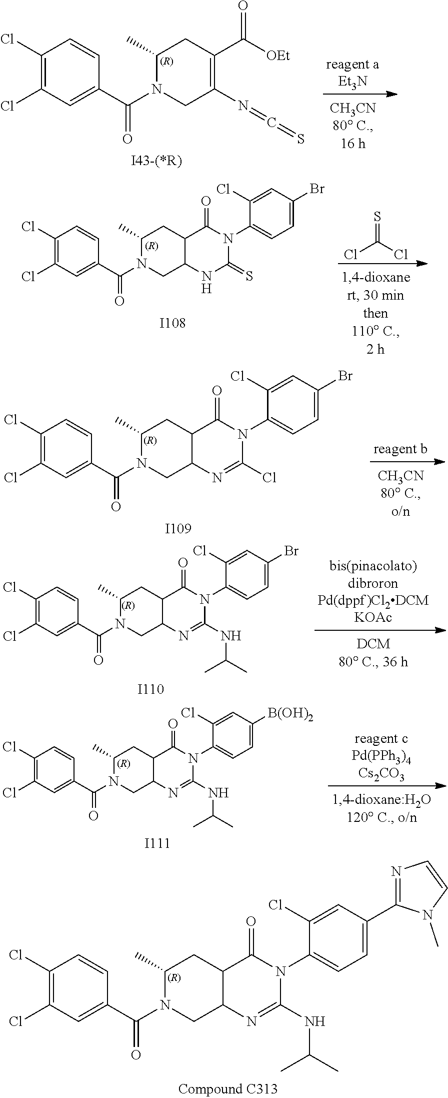

A microwave tube was charged with intermediate I43-(R) (3.00 g, 7.513 mmol), 4-bromo-2-chloroaniline (reagent a) (1.78 g, 8.27 mmol) and Et₃N (1.57 mL, 11.3 mmol) in dry CH₃CN (10 mL). The reaction mixture was stirred for at 80° C. for 16 h. The mixture was cooled to room temperature and the solvent was removed under reduced pressure. The crude mixture was purified by flash column chromatography (silica, mobile phase: heptane/EtOAc) to afford (6R)-3-(4-bromo-2-chlorophenyl)-7-(3,4-dichlorobenzoyl)-6-methyl-2-sulfanylidene-2,3,5,6,7,8-hexahydropyrido[3,4-d]pyrimidin-4(1H)-one (Intermediate I108) (3.8 g, 90%) as a white powder.

In a pressure tube, Intermediate I108 (3.8 g, 6.79 mmol) was dissolved in dry 1,4-dioxane (20 mL) and thiophosgene (0.73 mL, 9.51 mmol) was added. The tube was sealed under N₂ atmosphere and the mixture was stirred at room temperature for 30 min, then at 110° C. for 2 h. The reaction mixture was cooled to room temperature and loaded on a silica cartridge. The mixture was purified by flash column chromatography (silica, mobile phase gradient: 0 to 100% EtOAc in heptane) to afford (6R)-3-(4-bromo-2-chlorophenyl)-2-chloro-7-(3,4-dichlorobenzoyl)-6-methyl-5,6,7,8-tetrahydropyrido[3,4-d]pyrimidin-4(3H)-one (Intermediate I109) (3.1 g, 81%).

In a microwave tube, to a solution of Intermediate I109 (3.10 g, 5.52 mmol) in dry CH₃CN (30 mL) in a N₂ atmosphere was added isopropylamine (reagent b) (14 mL, 164 mmol). The tube was sealed and the reaction mixture was stirred at 80° C. overnight. The solvent was removed under reduced pressure. The crude mixture was purified by flash column chromatography (silica, mobile phase gradient: heptane/EtOAc) to afford (6R)-3-(4-bromo-2-chlorophenyl)-7-(3,4-dichlorobenzoyl)-6-methyl-2-[(propan-2-yl)amino]-5,6,7,8-tetrahydropyrido[3,4-d]pyrimidin-4(3H)-one (Intermediate I110) (2.75 g, 85%) as a white foam.

A mixture of Intermediate I110 (1.50 g, 2.57 mmol), bis(pinacolato)diboron (1.30 g, 5.13 mmol), KOAc (529 mg, 5.39 mmol) and Pd(dppf)Cl₂.DCM (212 mg, 0.26 mmol) in 1,4-dioxane (10 mL) was stirred at 85° C. for 4 days. The reaction was cooled to room temperature and the volatiles were removed under reduced pressure. The residue was purified by flash column chromatography (silica, mobile phase gradient: heptane/EtOAc) to afford {3-chloro-4-[(6R)-7-(3,4-dichlorobenzoyl)-6-methyl-4-oxo-2-[(propan-2-yl)amino]-5,6,7,8-tetrahydropyrido[3,4-d]pyrimidin-3(4H)-yl]phenyl}boronic acid (Intermediate I111) (633 mg, 45%).

Intermediate I111 (150 mg, 0.273 mmol), 2-bromo-1-methyl-1H-imidazole (reagent c) (87.9 mg, 0.55 mmol) and Pd(PPh₃)₄ (32.2 mg, 27.3 μmol) were dissolved in 1,4-dioxane (3 mL) and water (0.5 mL). The reaction mixture was stirred at 120° C. overnight. The reaction mixture was cooled to room temperature and diluted with water (2 mL) and 2-MeTHF (3 mL). The layers were separated and the organic phase was dried (MgSO₄), filtered and concentrated under reduced pressure to dryness. The crude mixture was purified by flash column chromatography (silica, mobile phase: heptane/EtOAc) to afford Compound C313 (100 mg, 63%).

### Procedure AN

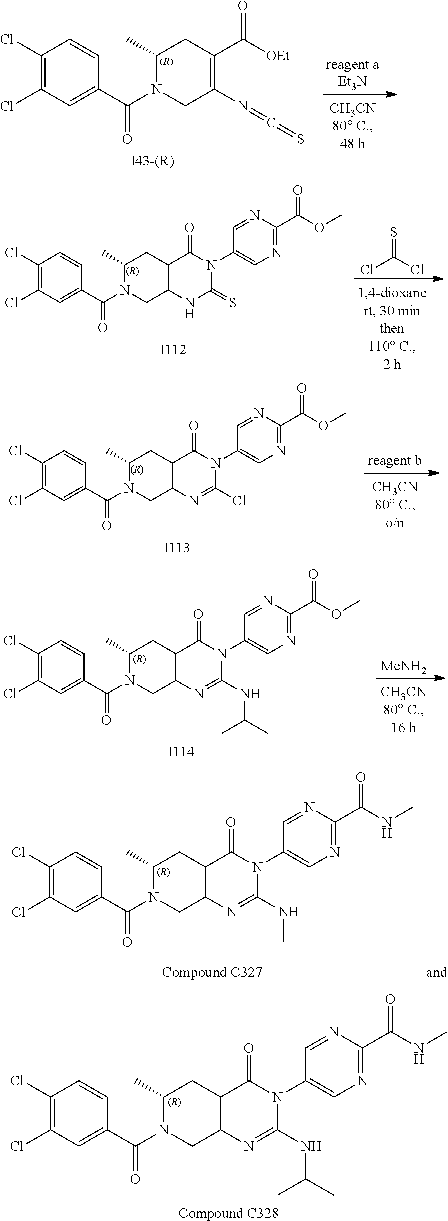

A mixture of Intermediate I43-(R) (2.20 g, 5.51 mmol), methyl 5-aminopyridine-2-carboxylate (reagent a) (1.00 g, 6.53 mmol) and Et₃N (2 mL, 14.4 mmol) in dry CH₃CN (25 mL) was stirred at 85° C. for 48 h. The solvent was removed under reduced pressure and the crude mixture was purified by flash column chromatography (silica, mobile phase gradient: from 0 to 100% EtOAc in heptane) to afford methyl 5-[(6R)-7-(3,4-dichlorobenzoyl)-6-methyl-4-oxo-2-sulfanylidene-1,4,5,6,7,8-hexahydropyrido[3,4-d]pyrimidin-3(2H)-yl]pyrimidine-2-carboxylate (Intermediate I112) (1.87 g, 67%) as a yellow powder.

In a pressure tube, Intermediate I112 (933 mg, 1.84 mmol) was solubilized in dry 1,4-dioxane (12 mL) and thiophosgene (0.24 mL, 3.13 mmol) was added under N₂ atmosphere. The reaction vessel was sealed and the reaction mixture was stirred at room temperature for 30 min, then at 110° C. for 1 h. The mixture was loaded on a silica cartridge and purified by flash column chromatography (silica, mobile phase gradient: 0 to 100% EtOAc in heptane) to afford methyl 5-[(6R)-2-chloro-7-(3,4-dichlorobenzoyl)-6-methyl-4-oxo-5,6,7,8-tetrahydropyrido[3,4-d]pyrimidin-3(4H)-yl]pyrimidine-2-carboxylate (Intermediate I113) (502 mg, 54%) as a light yellow powder.

In a microwave tube, to a solution of Intermediate I113 (502 mg, 0.99 mmol) in dry CH₃CN (11.5 mL) under N₂ atmosphere was added isopropylamine (reagent b) (0.17 mL, 1.98 mmol). The tube was sealed and the reaction mixture was stirred at 80° C. overnight. The solvent was evaporated under reduced pressure. The crude mixture was purified by flash column chromatography (silica, mobile phase gradient: 0 to 100% EtOAc in heptane, then 0 to 25% EtOH in EtOAc) to afford methyl 5-[(6R)-7-(3,4-dichlorobenzoyl)-6-methyl-4-oxo-2-[(propan-2-yl)amino]-5,6,7,8-tetrahydropyrido[3,4-d]pyrimidin-3(4H)-yl]pyrimidine-2-carboxylate (Intermediate I114) (400 mg, 51%, 66% purity) as a yellow powder.

In a microwave tube, Intermediate I114 (150 mg, 186 μmol, 66% purity) was dissolved in dry CH₃CN (1 mL) and methylamine (57.9 mg, 1.86 mmol) was added. The tube was sealed and the reaction mixture was stirred at 80° C. for 16 h. The mixture was purified by flash column chromatography (silica, mobile phase gradient: 0 to 10% MeOH in DCM) to afford Compound C328 (88 mg, 86%) as an off-white solid and non-pure fraction of Compound C327. The later was purified via SFC (Stationary phase: Chiralcel Diacel OJ 20×250 mm, Mobile phase: CO₂, EtOH+0.4% i-PrNH₂) to afford pure Compound C327 (19 mg, 20%) as a slightly yellow solid.

### Procedure AO

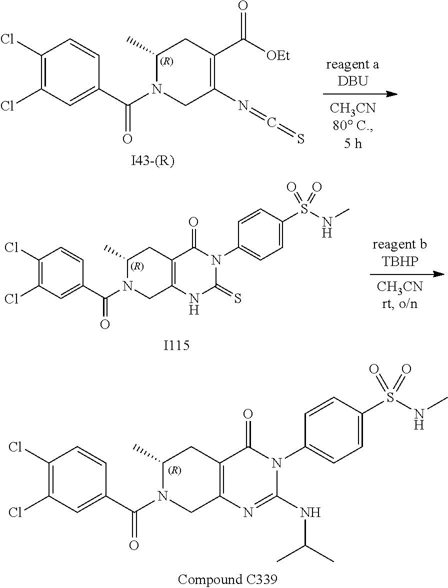

Intermediate I43-(R) (0.82 g, 2.07 mmol) was dissolved in CH₃CN (8.4 mL) and 4-amino-N-methylbenzenesulfonamide (reagent a) (500 mg, 2.69 mmol) and DBU (472 mg, 3.10 mmol) were added. The reaction mixture was stirred at 80° C. for 5 h. The solvent was removed under reduced pressure, and the residue was purified by flash column chromatography (silica, mobile phase gradient: 0 to 10% MeOH in DCM). A second purification via reverse phase HPLC delivered 4-[(6R)-7-(3,4-dichlorobenzoyl)-6-methyl-4-oxo-2-sulfanylidene-1,4,5,6,7,8-hexahydropyrido[3,4-d]pyrimidin-3(2H)-yl]-N-methylbenzene-1-sulfonamide (Intermediate I115) (281 mg, 25%) as a light yellow solid.

To a solution of Intermediate I115 (277 mg, 0.51 mmol), isopropylamine (reagent b) (265 μL, 3.08 mmol) in CH₃CN (1 mL) was added TBHP (0.28 mL, 1.54 mmol). The reaction mixture was stirred at room temperature overnight, and at 60° C. for 24 h. The solvent was evaporated under reduced pressure and the residue was purified by preparative RP-HPLC (CH₃CN/(NH₄)₂CO₃) to afford Compound C339 (89 mg, 31%) as a white solid.

The reaction mixture was stirred at room temperature overnight for the synthesis of Compounds C344 and C355.

For compound C344, the work-up was different: the reaction was quenched with Zn dust and the mixture was stirred for 30 minutes. The mixture was purified via preparative HPLC (Stationary phase: RP XBridge Prep C18 OBD-10 μm, 30×150 mm, Mobile phase: 0.25% NH₄HCO₃ solution in water, CH₃CN).

For compound C355, the work-up was different: the reaction was quenched with Zn dust and the mixture was stirred for 30 min. Volatiles were removed under reduced pressure. The residue was purified by flash column chromatography (silica, mobile phase gradient: 0 to 10% MeOH (with NH₃) in DCM). The residue was triturated in DIPE, filtered and dried under vacuum.

### Procedure AP

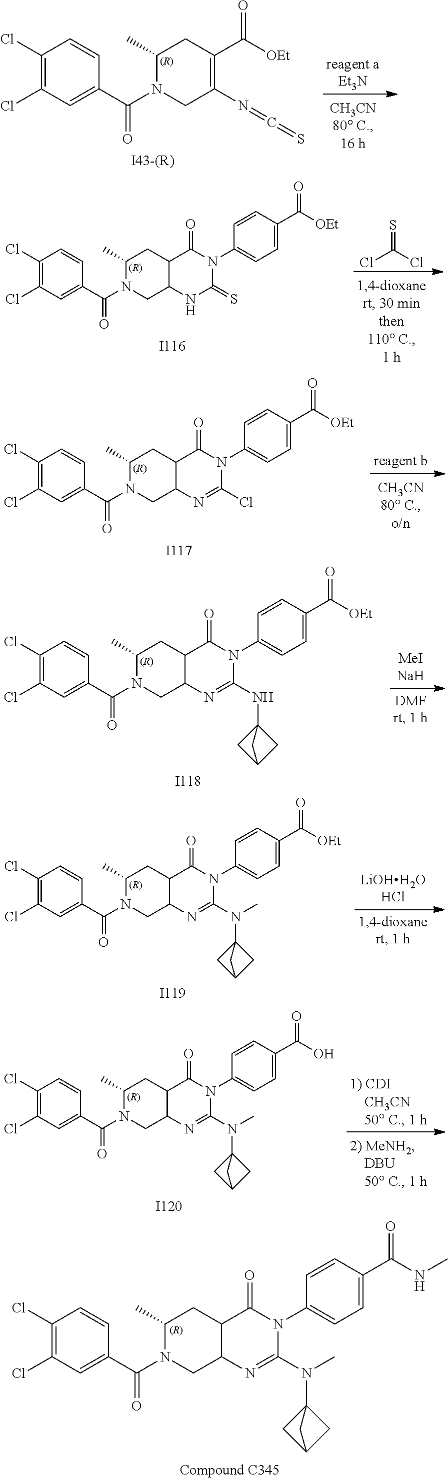

A pressure tube was charged with Intermediate I43-(R) (1.00 g, 2.50 mmol), ethyl 4-aminobenzoate (reagent a) (548.795 mg, 3.256 mmol), Et₃N (0.38 mL, 2.76 mmol) and in dry CH₃CN (7.6 mL). The reaction mixture was stirred at 80° C. for 16 h. The mixture was cooled to room temperature and concentrated under reduced pressure. The crude mixture was purified by flash column chromatography (silica, mobile phase gradient: 0 to 100% EtOAc in heptane). The residue was triturated in DIPE. The solid was filtered off and dried under vacuum to afford ethyl 4-[(6R)-7-(3,4-dichlorobenzoyl)-6-methyl-4-oxo-2-sulfanylidene-1,4,5,6,7,8-hexahydropyrido[3,4-d]pyrimidin-3(2H)-yl]benzoate (Intermediate I116) (1.1 g, 85%) as a pale yellow solid. A pressure tube was charged with Intermediate I116 (500 mg, 0.96 mmol) in dry 1,4-dioxane (4.9 mL). Thiophosgene (83.8 μL, 1.06 mmol) was added and the reaction mixture was stirred at room temperature for 30 min, then at 100° C. for 10 min. The mixture was concentrated under reduced pressure. The residue was purified by flash column chromatography (silica, mobile phase gradient: 0 to 100% EtOAc in heptane) to afford ethyl 4-[(6R)-2-chloro-7-(3,4-dichlorobenzoyl)-6-methyl-4-oxo-5,6,7,8-tetrahydropyrido[3,4-d]pyrimidin-3(4H)-yl]benzoate (Intermediate I117) (460 mg, 92%) as a yellow foam.

A pressure tube was charged with bicyclo[1.1.1]pentane-1-amine hydrochloride (reagent b) (171 mg, 1.36 mmol) and Et₃N (0.47 mL, 3.40 mmol) in dry CH₃CN (6.40 mL). The reaction mixture was stirred at room temperature for 5 min and Intermediate I117 (590 mg, 1.13 mmol) was added. The reaction mixture was stirred at 90° C. for 48 h. The mixture was cooled to room temperature and concentrated under reduced pressure. The residue was purified by flash column chromatography (silica, mobile phase gradient: 0 to 50% EtOAc in heptane) to afford ethyl 4-[(6R)-2-[(bicyclo[1.1.1]pentan-1-yl)amino]-7-(3,4-dichlorobenzoyl)-6-methyl-4-oxo-5,6,7,8-tetrahydropyrido[3,4-d]pyrimidin-3(4H)-yl]benzoate (Intermediate I118) (322 mg, 51%) as a white solid.

Intermediate I118 (322 mg, 567 μmol) was suspended in DMF (4 mL) under N₂ atmosphere. NaH (60% dispersion in mineral oil, 27.2 mg, 0.68 mmol) was added and the reaction mixture was stirred at for 10 min. Mel (38.9 μL, 0.62 mmol) was added dropwise and the reaction mixture was stirred at room temperature for 1 h. The mixture was poured out in cold water. The layers were separated and the aqueous phase was extracted with 2-MeTHF. The combined organic extracts were dried (MgSO₄), filtered and concentrated under reduced pressure. The residue was purified via Prep HPLC (Stationary phase: RP XBridge Prep C18 OBD-10 μm, 30×150 mm, Mobile phase: 0.25% NH₄HCO₃ solution in water, CH₃CN). The residue was dissolved in MeOH and concentrated under reduced pressure to afford ethyl 4-[(6R)-2-[(bicyclo[1.1.1]pentan-1-yl)(methyl)amino]-7-(3,4-dichlorobenzoyl)-6-methyl-4-oxo-5,6,7,8-tetrahydropyrido[3,4-d]pyrimidin-3(4H)-yl]benzoate (Intermediate I119) (150 mg, 45%).

Intermediate I119 (150 mg, 258 μmol) was dissolved in water (0.5 mL) and 1,4-dioxane (1.5 mL). Lithium hydroxide monohydrate (21.6 mg, 0.52 mmol) was added and the reaction mixture was stirred at room temperature for 1 h. The mixture was concentrated under reduced pressure and the residue was dissolved in water. HCl (1 M in H₂O, 0.52 mL, 0.52 mmol) was added and the mixture was extracted with 2-MeTHF. The combined organic extracts were concentrated under reduced pressure to afford 4-[(6R)-2-[(bicyclo[1.1.1]pentan-1-yl)(methyl)amino]-7-(3,4-dichlorobenzoyl)-6-methyl-4-oxo-5,6,7,8-tetrahydropyrido[3,4-d]pyrimidin-3(4H)-yl]benzoic acid (intermediate I120) (130 mg, 91%).

A pressure tube was charged with Intermediate I120 (130 mg, 235 μmol) in CH₃CN (4.1 mL). CDI (57.1 mg, 0.35 mmol) was added and the reaction mixture was stirred at 50° C. for 1 h. The mixture was cooled to room temperature and methylamine (8.75 mg, 0.28 mmol) and DBU (70.2 μL, 0.47 mmol) was added. The reaction mixture was stirred at 50° C. for 1 h. The mixture was cooled to room temperature and concentrated under reduced pressure. The crude mixture was purified by flash column chromatography (silica, mobile phase gradient: 0 to 100% EtOAc/EtOH (2:1) in heptane). The residue was triturated in DIPE, filtered off and dried under vacuum to afford Compound C345 (88 mg, 66%) as a white solid.

### Procedure AQ

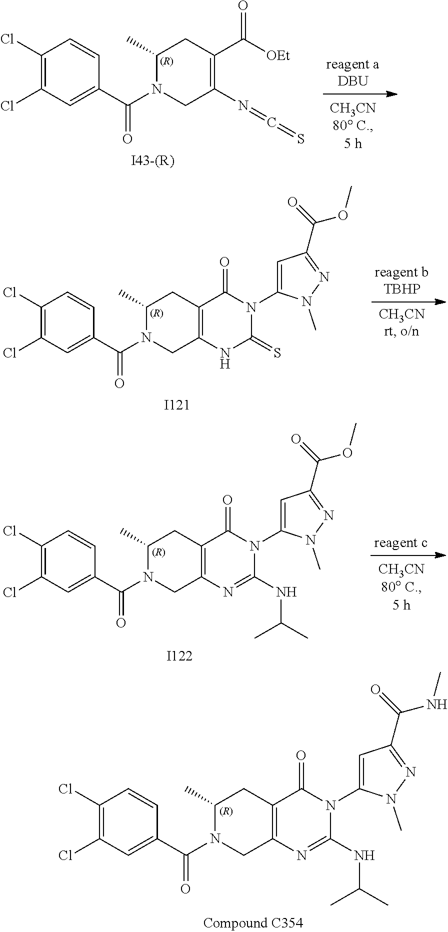

Intermediate I43-(R) (990 mg, 2.48 mmol) was dissolved in CH₃CN (9 ml-) and methyl 5-amino-1-methyl-1H-pyrazole-3-carboxylate (reagent a) (500 mg, 3.22 mmol) and DBU (566 mg, 3.72 mmol) were added. The reaction mixture was stirred at 80° C. for 5 h. The solvent was removed under reduced pressure and the residue was purified by flash column chromatography (silica, mobile phase gradient: 0 to 5% MeOH in DCM) to afford methyl 5-[(6R)-7-(3,4-dichlorobenzoyl)-6-methyl-4-oxo-2-sulfanylidene-1,4,5,6,7,8-hexahydropyrido[3,4-d]pyrimidin-3(2H)-yl]-1-methyl-1H-pyrazole-3-carboxylate (Intermediate I121) (470 mg, 37%).

To a mixture of Intermediate I121 (450 mg, 0.88 mmol), isopropylamine (reagent b) (456 μL, 5.31 mmol) in CH₃CN (8 mL) was added TBHP (368 μL, 2.66 mmol). The reaction mixture was stirred at room temperature for 48 h. The reaction was quenched with a saturated aqueous solution of Na₂S₂O₃. The solvent was removed under reduced pressure and the aqueous phase was extracted with EtOAc. The combined organic extracts were washed with brine, dried (MgSO₄), filtered and concentrated under reduced pressure. The residue was purified by flash column chromatography (silica, mobile phase gradient: 0 to 3% MeOH (with NH₃) in DCM) to afford methyl 5-[(6R)-7-(3,4-dichlorobenzoyl)-6-methyl-4-oxo-2-[(propan-2-yl)amino]-5,6,7,8-tetrahydropyrido[3,4-d]pyrimidin-3(4H)-yl]-1-methyl-1H-pyrazole-3-carboxylate (Intermediate I122) (164 mg, 35%) as light brownish gum.

To the solution of Intermediate I122 (160 mg, 0.30 mmol) in CH₃CN (5 mL) was added methylamine (reagent c) (93.2 mg, 3.00 mmol). The reaction mixture was stirred at 80° C. for 5 h. The solvent was removed under reduced pressure and the crude mixture was purified by flash column chromatography (silica, mobile phase gradient: 0 to 10% MeOH (with NH₃) in DCM) to afford Compound C354 (105 mg, 66%) as a light orange solid.

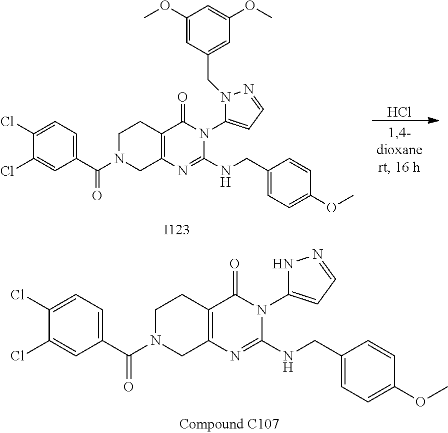 [SMILES: "I123" ; COc1ccc(CNc2nc3c(c(=O)n2-c2ccnn2Cc2cc(OC)cc(OC)c2)CCN(C(=O)c2ccc(Cl)c(Cl)c2)C3)cc1 ; COc1ccc(CNc2nc3c(c(=O)n2-c2ccn[nH]2)CCN(C(=O)c2ccc(Cl)c(Cl)c2)C3)cc1 ; Cl]

A mixture of 7-(3,4-dichlorobenzoyl)-3-{1-[(3,5-dimethoxyphenyl)methyl]-1H-pyrazol-5-yl}-2-{[(4-methoxyphenyl)methyl]amino}-5,6,7,8-tetrahydropyrido[3,4-d]pyrimidin-4(3H)-one (Intermediate I123) (131 mg, 0.19 mmol) and HCl (4M in 1,4-dioxane, 2.5 mL, 10 mmol) in dry 1,4-dioxane (1.5 mL) was stirred at room temperature for 16 h. The reaction mixture was concentrated under reduced pressure. The crude mixture was purified via Prep HPLC (Stationary phase: RP XBridge Prep C18 OBD-10 μm, 50×150 mm, Mobile phase: 0.25% NH₄HCO₃ solution in water, CH₃CN). The residue was dissolved in MeOH and concentrated under reduced pressure. The product was triturated in DIPE, filtered off and dried under vacuum to give Compound C107 (27 mg, 27%) as a yellow solid.

For Compound C180, the residue was partitioned between 2-MeTHF and a saturated aqueous solution of Na₂CO₃. The layers were separated and the organic phase was dried (MgSO₄), filtered and concentrated under reduced pressure. The residue was finally purified by via Preparative HPLC (Stationary phase: RP XBridge Prep C18 OBD-10 μm, 30×150 mm, Mobile phase: 0.25% NH₄HCO₃ solution in water, CH₃CN). Compound C184 was purified via preparative SFC (Stationary phase: Chiralpak Diacel AS 20×250 mm, Mobile phase: CO₂, MeOH+0.4% i-PrNH₂).

### Procedure AS

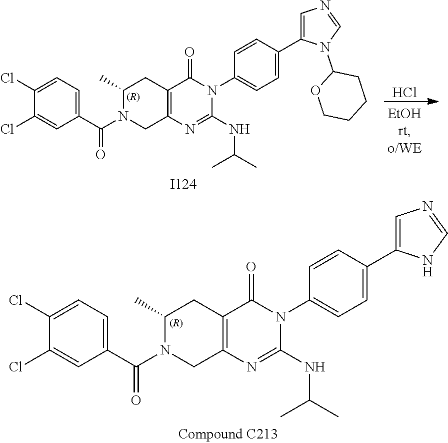

HCl (6M in i-PrOH, 5 mL, 30 mmol) was added to a solution of (6R)-7-(3,4-dichloro-benzoyl)-6-methyl-3-{4-[1-(oxan-2-yl)-1H-imidazol-5-yl]phenyl}-2-[(propan-2-yl)amino]-5,6,7,8-tetrahydropyrido[3,4-d]pyrimidin-4(3H)-one (Intermediate I124) (152 mg, 0.24 mmol) in EtOH (5 mL). The reaction mixture was stirred at room temperature over the weekend. The volatiles were removed under reduced pressure and the residue was purified by flash column chromatography (silica, mobile phase gradient: 0 to 100% (EtOAc/EtOH/NH₃, 3:1:0.02) in heptane to give Compound C213 (125 mg, 96%).

The solvent of the reaction was i-PrOH and the reaction was performed at 100° C. for 20 min in the synthesis of Compounds C263 and C315.

### 4. List of Synthesized Compounds

The compounds listed below have been purified by preparative SFC and/or by preparative HPLC following one of the methods described in general information “Separation of the rotamers, diastereoisomers and enantiomers”: Compounds C26a, C26b, C36Aa, C26Ba, C36Aa, C36Bb, C62A, C62Ba, C62Bb, C63A, C63B, C100A, C100B, C101a, C101b, C109A, C109B, C110A, C110B, C154A, C154B, C158A, C158B, C256, C257, C282, C283, C285, C286, C294, C295, C300a, C300b, C308a, C308b, C311, C312, C314a, C314b, C318, C319, C320, C321, C340a, C340b, C342, C343, C348, C349, C350, C351, C352, C353.

In the synthesis of Compound C287, procedure U, the reaction with thiophosgene between the reactions with reagents a and b was not carried out.

| | Name | | |
|---|---|---|---|
| Compound | | Starting | |
| # | Procedure | material | Reagents |
| C1 | 7-(3,4-dichlorobenzoyl)-3-(2-methoxyphenyl)-2-{[(4- | | |
| | methoxyphenyl)methyl]amino}-5,6,7,8-tetrahydropyrido[3,4-d]pyrimidin- | | |
| | 4(3H)-one | | |
| | A | I7a | a: 3,4-dichlorobenzoyl chloride, conditions A |
| C2 | 7-(1H-indole-2-carbonyl)-2-((4-methoxybenzyl)amino)-3-(2-methoxyphenyl)- | | |
| | 5,6,7,8-tetrahydropyrido[3,4-d]pyrimidin-4(3H)-one | | |
| | A | I7a | a: indole-2-carboxylic acid, conditions B |
| C3 | 7-(3-chloro-4-fluorobenzoyl)-3-(2-methoxyphenyl)-2-{[(4-methoxyphenyl)- | | |
| | methyl]amino}-5,6,7,8-tetrahydropyrido[3,4-d]pyrimidin-4(3H)-one | | |
| | A | I7a | a: 3-chloro-4-fluorobenzoic acid, conditions B |
| C4 | 7-(3,4-dichlorobenzoyl)-3-(2-methoxyphenyl)-2-(methylsulfanyl)-5,6,7,8- | | |
| | tetrahydropyrido[3,4-d]pyrimidin-4(3H)-one | | |
| | Compound C4 is an intermediate in the synthesis of Intermediates 11a, 11b | | |
| | and 11c. | | |
| C5 | 7-(3,4-dichlorobenzoyl)-3-(2-methoxyphenyl)-2-{[(Pyridin-4-yl)methyl]amino}- | | |
| | 5,6,7,8-tetrahydropyrido[3,4-d]pyrimidin-4(3H)-one | | |
| | B | I11a | a: 4-picolylamine, conditions A |
| C6 | 7-(3,4-dichlorobenzoyl)-2-(methylsulfanyl)-5,6,7,8-tetrahydropyrido[3,4- | | |
| | d]pyrimidin-4(3H)-one | | |
| | D | I14 | |
| C7 | 7-(3,4-dichlorobenzoyl)-2-{[(4-methoxyphenyl)methyl]amino}-3-[3- | | |
| | (trifluoromethyl)phenyl]-5,6,7,8-tetrahydropyrido[3,4-d]pyrimidin-4(3H)-one | | |
| | A | I7b | a: 3,4-dichlorobenzoyl chloride, conditions A |
| C8 | 7-(3,4-dichlorobenzoyl)-3-(3-methoxyphenyl)-2-{[(4-methoxyphenyl)- | | |
| | methyl]amino}-5,6,7,8-tetrahydropyrido[3,4-d]pyrimidin-4(3H)-one | | |
| | A | I7c | a: 3,4-dichlorobenzoyl chloride, conditions A |
| C9 | 7-(3,4-dichlorobenzoyl)-3-(2-methoxyphenyl)-2-{[(pyridin-3-yl)methyl]amino}- | | |
| | 5,6,7,8-tetrahydropyrido[3,4-d]pyrimidin-4(3H)-one | | |
| | B | I11a | a: 3-(aminomethyl)pyridine, conditions A |
| C10 | 2-(benzylamino)-7-(3,4-dichlorobenzoyl)-3-(2-methoxyphenyl)-5,6,7,8- | | |
| | tetrahydropyrido[3,4-d]pyrimidin-4(3H)-one | | |
| | B | I11a | a: benzylamine, conditions A |
| C11 | 7-(3,4-dichlorobenzoyl)-3-(2-methoxyphenyl)-2-(methylamino)-5,6,7,8- | | |
| | tetrahydropyrido[3,4-d]pyrimidin-4(3H)-one | | |
| | B | I11a | a: methylamine, conditions A |
| C12 | 7-(3,4-dichlorobenzoyl)-3-(2-methoxyphenyl)-2-{[(pyridin-2-yl)methyl]amino}- | | |
| | 5,6,7,8-tetrahydropyrido[3,4-d]pyrimidin-4(3H)-one | | |
| | B | I11a | a: 2-(aminomethyl)pyridine, conditions A |
| C13 | 7-(3,4-dichlorobenzoyl)-3-(2-methoxyphenyl)-2-{[(1,3-thiazol-2- | | |
| | yl)methyl]amino}-5,6,7,8-tetrahydropyrido[3,4-d]pyrimidin-4(3H)-one | | |
| | B | I11a | a: thiazol-2-ylmethanamine, conditions A |
| C14 | 7-(3,4-dichlorobenzoyl)-3-(2-fluorophenyl)-2-{[(4-methoxyphenyl)methyl]- | | |
| | amino}-5,6,7,8-tetrahydropyrido[3,4-d]pyrimidin-4(3H)-one | | |
| | B | I11b | a: 4-methoxybenzylamine, conditions A |
| C15 | 3-(2-chlorophenyl)-7-(3,4-dichlorobenzoyl)-2-{[(4-methoxyphenyl)methyl]- | | |
| | amino}-5,6,7,8-tetrahydropyrido[3,4-d]pyrimidin-4(3H)-one | | |
| | B | I11c | a: 4-methoxybenzylamine, conditions A |
| C16 | 7-(3,4-dichlorobenzoyl)-3-(2-methoxyphenyl)-2-{[(1,3-thiazol-4- | | |
| | yl)methyl]amino}-5,6,7,8-tetrahydropyrido[3,4-d]pyrimidin-4(3H)-one | | |
| | B | I11a | a: 1,3-thiazol-4-ylmethanamine, conditions A |
| C17 | 7-(3,4-dichlorobenzoyl)-3-(2-methoxyphenyl)-2-{[(2-methoxyphenyl)methyl]- | | |
| | amino}-5,6,7,8-tetrahydropyrido[3,4-d]pyrimidin-4(3H)-one | | |
| | B | I11a | a: 2-methoxybenzylamine, conditions A |
| C18 | 7-(3,4-dichlorobenzoyl)-3-(2-methoxyphenyl)-2-{[(3-methoxyphenyl)methyl]- | | |
| | amino}-5,6,7,8-tetrahydropyrido[3,4-d]pyrimidin-4(3H)-one | | |
| | B | I11a | a: 3-methoxybenzylamine, conditions A |
| C19 | 7-(3,4-dichlorobenzoyl)-3-(2-methoxyphenyl)-2-{[(1,3-thiazol-5- | | |
| | yl)methyl]amino}-5,6,7,8-tetrahydropyrido[3,4-d]pyrimidin-4(3H)-one | | |
| | B | I11a | a: thiazol-5-ylmethanamine, conditions B |
| C20 | 7-(3,4-dichlorobenzoyl)-3-(2-methoxyphenyl)-2-[(2-phenylethyl)amino]- | | |
| | 5,6,7,8-tetrahydropyrido[3,4-d]pyrimidin-4(3H)-one | | |
| | B | I11a | a: 2-phenylethylamine, conditions B |
| C21 | 2-(benzylamino)-7-(3,4-dichlorobenzoyl)-5,6,7,8-tetrahydropyrido[3,4- | | |
| | d]pyrimidin-4(3H)-one | | |
| | E | I13 | a: N-benzylguanidine, b: t-BuOK |
| C22 | 7-(3,4-dichlorobenzoyl)-3-(2-methoxyphenyl)-2-[(2-methylpropyl)amino]- | | |
| | 5,6,7,8-tetrahydropyrido[3,4-d]pyrimidin-4(3H)-one | | |
| | B | I11a | a: isobutylamine, conditions B |
| C23 | 7-(3,4-dichlorobenzoyl)-3-(2-methoxyphenyl)-2-({[4-(trifluoromethyl)phenyl]- | | |
| | methyl}amino)-5,6,7,8-tetrahydropyrido[3,4-d]pyrimidin-4(3H)-one | | |
| | B | I11a | a: 4-(trifluoromethyl)benzylamine, conditions A |
| C24 | 7-(3,4-dichlorobenzoyl)-3-(2-methoxyphenyl)-2-[(propan-2-yl)amino]-5,6,7,8- | | |
| | tetrahydropyrido[3,4-d]pyrimidin-4(3H)-one | | |
| | B | I11a | a: isopropylamine, conditions C |
| C25 | 2-{[(1-acetylpiperidin-4-yl)methyl]amino}-7-(3,4-dichlorobenzoyl)-3-(2- | | |
| | methoxyphenyl)-5,6,7,8-tetrahydropyrido[3,4-d]pyrimidin-4(3H)-one | | |
| | B | I11a | a: 1-[4-(aminomethyl)piperidin-1-yl]ethan-1-one, |
| | | | conditions B |
| C26 | 2-{[(2H-1,3-benzodioxol-5-yl)methyl]amino}-7-(3,4-dichlorobenzoyl)-3-(2- | | |
| | methoxyphenyl)-5,6,7,8-tetrahydropyrido[3,4-d]pyrimidin-4(3H)-one | | |
| | B | I11a | a: piperonylamine, conditions C |
| C26a | 2-{[(2H-1,3-benzodioxol-5-yl)methyl]amino}-7-(3,4-dichlorobenzoyl)-3-(2- | | |
| | methoxyphenyl)-5,6,7,8-tetrahydropyrido[3,4-d]pyrimidin-4(3H)-one - | | |
| | Rotamer (*S) | | |
| C26b | 2-{[(2H-1,3-benzodioxol-5-yl)methyl]amino}-7-(3,4-dichlorobenzoyl)-3-(2- | | |
| | methoxyphenyl)-5,6,7,8-tetrahydropyrido[3,4-d]pyrimidin-4(3H)-one - | | |
| | Rotamer (*R) | | |
| C27 | 7-(3,4-dichlorobenzoyl)-2-({[4-(methanesulfonyl)phenyl]methyl}amino)-3-(2- | | |
| | methoxyphenyl)-5,6,7,8-tetrahydropyrido[3,4-d]pyrimidin-4(3H)-one | | |
| | B | I11a | a: 4-(methylsulphonyl)benzylamine hydrochloride, |
| | | | conditions C |
| C28 | 2-(benzylamino)-7-(3,4-dichlorobenzoyl)-3-(2-methoxyethyl)-5,6,7,8- | | |
| | tetrahydropyrido[3,4-d]pyrimidin-4(3H)-one | | |
| | E | C21 | c: 2-bromoethyl methyl ether, conditions A |
| C29 | 7-(3,4-dichlorobenzoyl)-2-{[(2,4-difluorophenyl)methyl]amino}-3-(2- | | |
| | methoxyphenyl)-5,6,7,8-tetrahydropyrido[3,4-d]pyrimidin-4(3H)-one | | |
| | C | I12 | a: 2,4-difluorobenzylamine, conditions A |
| C30 | 7-(3,4-dichlorobenzoyl)-2-{[(2,4-dichlorophenyl)methyl]amino}-3-(2- | | |
| | methoxyphenyl)-5,6,7,8-tetrahydropyrido[3,4-d]pyrimidin-4(3H)-one | | |
| | C | I12 | a: 2,4-dichlorobenzylamine, conditions A |
| C31 | 7-(3,4-dichlorobenzoyl)-2-{[(4-methoxyphenyl)methyl](methyl)amino}- | | |
| | 5,6,7,8-tetrahydropyrido[3,4-d]pyrimidin-4(3H)-one | | |
| | E | I13 | a: N-(4-methoxybenzyl)-N-methylguanidine sulfate, b: t- |
| | | | BuOK |
| C32 | 2-(cyclobutylamino)-7-(3,4-dichlorobenzoyl)-3-(2-methoxyphenyl)-5,6,7,8- | | |
| | tetrahydropyrido[3,4-d]pyrimidin-4(3H)-one | | |
| | C | I12 | a: cyclobutanamine, conditions C |
| C33 | 2-(benzylamino)-7-(3,4-dichlorobenzoyl)-3-phenyl-5,6,7,8- | | |
| | tetrahydropyrido[3,4-d]pyrimidin-4(3H)-one | | |
| | E | C21 | c: phenylboronic acid, conditions B |
| C34 | 7-(3,4-dichlorobenzoyl)-2-{[(4-methoxyphenyl)methyl]amino}-5,6,7,8- | | |
| | tetrahydropyrido[3,4-d]pyrimidin-4(3H)-one | | |
| | F | I15 | a: 4-methoxybenzylamine |
| C35 | 7-(3,4-dichlorobenzoyl)-3-(2-methoxyphenyl)-2-{[(1S)-1-phenylethyl]amino}- | | |
| | 5,6,7,8-tetrahydropyrido[3,4-d]pyrimidin-4(3H)-one | | |
| | C | I12 | a: (S)-(−)-1-phenylethylamine, conditions B |
| C36 | 7-(3,4-dichlorobenzoyl)-3-(2-methoxyphenyl)-2-{[(4-methoxyphenyl)methyl]- | | |
| | amino}-6-methyl-5,6,7,8-tetrahydropyrido[3,4-d]pyrimidin-4(3H)-one | | |
| | H | I23 | a: 4-methoxybenzylamine |
| C36Aa | (6R)-7-(3,4-dichlorobenzoyl)-3-(2-methoxyphenyl)-2-{[(4-methoxyphenyl)- | | |
| | methyl]amino}-6-methyl-5,6,7,8-tetrahydropyrido[3,4-d]pyrimidin-4(3H)-one - | | |
| | Enantiomer (R), rotamer (*R) | | |
| C36Ba | (6S)-7-(3,4-dichlorobenzoyl)-3-(2-methoxyphenyl)-2-{[(4-methoxyphenyl)- | | |
| | methyl]amino}-6-methyl-5,6,7,8-tetrahydropyrido[3,4-d]pyrimidin-4(3H)-one - | | |
| | Enantiomer (S), rotamer (*R) | | |
| C36Ab | (6R)-7-(3,4-dichlorobenzoyl)-3-(2-methoxyphenyl)-2-{[(4-methoxyphenyl)- | | |
| | methyl]amino}-6-methyl-5,6,7,8-tetrahydropyrido[3,4-d]pyrimidin-4(3H)-one - | | |
| | Enantiomer (R), rotamer (*S) | | |
| C36Bb | (6S)-7-(3,4-dichlorobenzoyl)-3-(2-methoxyphenyl)-2-{[(4-methoxyphenyl)- | | |
| | methyl]amino}-6-methyl-5,6,7,8-tetrahydropyrido[3,4-d]pyrimidin-4(3H)-one - | | |
| | Enantiomer (S), rotamer (*S) | | |
| C37 | 7-(3,4-dichlorobenzoyl)-2-[(2-hydroxyethyl)amino]-3-(2-methoxyphenyl)- | | |
| | 5,6,7,8-tetrahydropyrido[3,4-d]pyrimidin-4(3H)-one | | |
| | C | I12 | a: ethanolamine, conditions B |
| C38 | 7-(3,4-dichlorobenzoyl)-2-(ethylamino)-3-(2-methoxyphenyl)-5,6,7,8- | | |
| | tetrahydropyrido[3,4-d]pyrimidin-4(3H)-one | | |
| | C | I12 | a: ethylamine, conditions B |
| C39 | 7-(3,4-dichlorobenzoyl)-3-(2-methoxyphenyl)-2-(pyrrolidin-1-yl)-5,6,7,8- | | |
| | tetrahydropyrido[3,4-d]pyrimidin-4(3H)-one | | |
| | C | I12 | a: pyrrolidine, conditions B |
| C40 | 7-(3,4-dichlorobenzoyl)-3-(2-methoxyphenyl)-2-(morpholin-4-yl)-5,6,7,8- | | |
| | tetrahydropyrido[3,4-d]pyrimidin-4(3H)-one | | |
| | C | I12 | a: morpholine, conditions B |
| C41 | 3-benzyl-7-(3,4-dichlorobenzoyl)-2-{[(4-methoxyphenyl)methyl]amino}- | | |
| | 5,6,7,8-tetrahydropyrido[3,4-d]pyrimidin-4(3H)-one | | |
| | F | C34 | b: benzyl bromide |
| C42 | 7-(3,4-dichlorobenzoyl)-2-[(propan-2-yl)amino]-5,6,7,8-tetrahydropyrido[3,4- | | |
| | d]pyrimidin-4(3H)-one | | |
| | E | I13 | a: bis(1-(propan-2-yl)guanidine) sulfuric acid, b: DBU |
| C43 | 7-(3,4-dichlorobenzoyl)-3-(2-methoxyphenyl)-2-{[(4-methoxyphenyl)methyl]- | | |
| | (methyl)amino}-5,6,7,8-tetrahydropyrido[3,4-d]pyrimidin-4(3H)-one | | |
| | C | I12 | a: 4-methoxy-N-methylbenzylamine, conditions B |
| C44 | 7-(3,4-dichlorobenzoyl)-3-(2-methoxyphenyl)-2-{[(1R)-1-phenylethyl]amino}- | | |
| | 5,6,7,8-tetrahydropyrido[3,4-d]pyrimidin-4(3H)-one | | |
| | C | I12 | a: (R)-(+)-1-phenylethylamine, conditions B |
| C45 | 2-(cyclopentylamino)-7-(3,4-dichlorobenzoyl)-3-(2-methoxyphenyl)-5,6,7,8- | | |
| | tetrahydropyrido[3,4-d]pyrimidin-4(3H)-one | | |
| | C | I12 | a: cyclopentylamine, conditions B |
| C46 | 7-(3,4-dichlorobenzoyl)-2-(dimethylamino)-3-(2-methoxyphenyl)-5,6,7,8- | | |
| | tetrahydropyrido[3,4-d]pyrimidin-4(3H)-one | | |
| | C | I12 | a: dimethylamine, conditions B |
| C47 | 2-[(1-cyclopropylethyl)amino]-7-(3,4-dichlorobenzoyl)-3-(2-methoxyphenyl)- | | |
| | 5,6,7,8-tetrahydropyrido[3,4-d]pyrimidin-4(3H)-one | | |
| | C | I12 | a: 1-cyclopropylethanamine, conditions B |
| C48 | 7-(3,4-dichlorobenzoyl)-3-(2-methoxyphenyl)-2-[(oxetan-3-yl)amino]-5,6,7,8- | | |
| | tetrahydropyrido[3,4-d]pyrimidin-4(3H)-one | | |
| | C | I12 | a: 3-oxetanamine, conditions B |
| C49 | 2-[(butan-2-yl)amino]-7-(3,4-dichlorobenzoyl)-3-(2-methoxyphenyl)-5,6,7,8- | | |
| | tetrahydropyrido[3,4-d]pyrimidin-4(3H)-one | | |
| | C | I12 | a: sec-butylamine, conditions C |
| C50 | 2-[(cyclopropylmethyl)amino]-7-(3,4-dichlorobenzoyl)-3-(2-methoxyphenyl)- | | |
| | 5,6,7,8-tetrahydropyrido[3,4-d]pyrimidin-4(3H)-one | | |
| | C | I12 | a: cyclopropylmethylamine, conditions C |
| C51 | 7-(3,4-dichlorobenzoyl)-3-(2-methoxyethyl)-2-{[(4-methoxyphenyl)methyl]- | | |
| | amino}-5,6,7,8-tetrahydropyrido[3,4-d]pyrimidin-4(3H)-one | | |
| | F | C34 | b: 2-bromoethyl methyl ether |
| C52 | 7-(3,4-dichlorobenzoyl)-2-{[(4-methoxyphenyl)methyl]amino}-3-methyl- | | |
| | 5,6,7,8-tetrahydropyrido[3,4-d]pyrimidin-4(3H)-one | | |
| | F | C34 | b: methyl iodide |
| C53 | 2-(tert-butylamino)-7-(3,4-dichlorobenzoyl)-3-(2-methoxyphenyl)-5,6,7,8- | | |
| | tetrahydropyrido[3,4-d]pyrimidin-4(3H)-one | | |
| | C | I12 | a: tert-butylamine, conditions B |
| C54 | 7-(3,4-dichlorobenzoyl)-2-methoxy-3-(2-methoxyphenyl)-5,6,7,8- | | |
| | tetrahydropyrido[3,4-d]pyrimidin-4(3H)-one | | |
| | C | I12 | a: NaOMe, conditions D |
| C55 | 7-(3,4-dichlorobenzoyl)-2-[(2,3-dihydro-1H-inden-1-yl)amino]-3-(2- | | |
| | methoxyphenyl)-5,6,7,8-tetrahydropyrido[3,4-d]pyrimidin-4(3H)-one | | |
| | C | I12 | a: 1-aminoindan, conditions C |
| C56 | 7-(3,4-dichlorobenzoyl)-3-(2-methoxyphenyl)-2-{[1-(pyridin-4-yl)ethyl]amino}- | | |
| | 5,6,7,8-tetrahydropyrido[3,4-d]pyrimidin-4(3H)-one | | |
| | C | I12 | a: 1-(pyridin-4-yl)ethanamine, conditions C |
| C57 | 2-amino-7-(3,4-dichlorobenzoyl)-3-(2-methoxyphenyl)-5,6,7,8- | | |
| | tetrahydropyrido[3,4-d]pyrimidin-4(3H)-one | | |
| | C | I12 | a: NH₃, conditions B |
| C58 | 7-(3,4-dichlorobenzoyl)-2-{[(4-methoxyphenyl)methyl]amino}-3-(oxetan-3-yl)- | | |
| | 5,6,7,8-tetrahydropyrido[3,4-d]pyrimidin-4(3H)-one | | |
| | F | C34 | b: 3-iodooxetane |
| C59 | 3-(cyclopropylmethyl)-7-(3,4-dichlorobenzoyl)-2-{[(4-methoxyphenyl)methyl]- | | |
| | amino}-5,6,7,8-tetrahydropyrido[3,4-d]pyrimidin-4(3H)-one | | |
| | F | C34 | b: (bromomethyl)cyclopropane |
| C60 | 7-(4-chloro-3-methylbenzoyl)-2-{[(4-methoxyphenyl)methyl]amino}-5,6,7,8- | | |
| | tetrahydropyrido[3,4-d]pyrimidin-4(3H)-one | | |
| | I | I26 | a: 4-chloro-3-methylbenzoic acid; conditions A |
| C61 | 7-(4-bromo-3-chlorobenzoyl)-2-{[(4-methoxyphenyl)methyl]amino}-5,6,7,8- | | |
| | tetrahydropyrido[3,4-d]pyrimidin-4(3H)-one | | |
| | I | I26 | a: 4-bromo-3-fluorobenzoyl chloride; conditions B |
| C62A | 7-(3,4-dichlorobenzoyl)-2-{[(2*R)-1-hydroxypropan-2-yl]amino}-3-(2- | | |
| | methoxyphenyl)-5,6,7,8-tetrahydropyrido[3,4-d]pyrimidin-4(3H)-one - | | |
| | Diastereomer (*R) | | |
| | C | I12 | a: DL-alaninol, conditions B |
| C62Ba | 7-(3,4-dichlorobenzoyl)-2-{[(2*S)-1-hydroxypropan-2-yl]amino}-3-(2- | | |
| | methoxyphenyl)-5,6,7,8-tetrahydropyrido[3,4-d]pyrimidin-4(3H)-one - | | |
| | Diastereomer (*S), Rotamer (*R) | | |
| C62Bb | 7-(3,4-dichlorobenzoyl)-2-{[(2*S)-1-hydroxypropan-2-yl]amino}-3-(2- | | |
| | methoxyphenyl)-5,6,7,8-tetrahydropyrido[3,4-d]pyrimidin-4(3H)-one - | | |
| | Diastereomer (*S), Rotamer (*S) | | |
| C63 | 7-(3,4-dichlorobenzoyl)-2-{[(4-methoxyphenyl)methyl]amino}-6-methyl- | | |
| | 5,6,7,8-tetrahydropyrido[3,4-d]pyrimidin-4(3H)-one | | |
| | E | I20 | a: 1-(4-methoxybenzyl)guanidine, b: DBU |
| C63A | (6R)-7-(3,4-dichlorobenzoyl)-2-{[(4-methoxyphenyl)methyl]amino}-6-methyl- | | |
| | 5,6,7,8-tetrahydropyrido[3,4-d]pyrimidin-4(3H)-one - Enantiomer (R) | | |
| C63B | (6S)-7-(3,4-dichlorobenzoyl)-2-{[(4-methoxyphenyl)methyl]amino}-6-methyl- | | |
| | 5,6,7,8-tetrahydropyrido[3,4-d]pyrimidin-4(3H)-one - Enantiomer (S) | | |
| C64 | 3-cyclohexyl-7-(3,4-dichlorobenzoyl)-2-{[(4-methoxyphenyl)methyl]amino}- | | |
| | 5,6,7,8-tetrahydropyrido[3,4-d]pyrimidin-4(3H)-one | | |
| | G | I13 | a: 1-cyclohexyl-2-thiourea; b: 4-methoxybenzylamine |
| C65 | 7-(3,4-dichlorobenzoyl)-2-{[(4-methoxyphenyl)methyl]amino}-3-[(oxan-4- | | |
| | yl)methyl]-5,6,7,8-tetrahydropyrido[3,4-d]pyrimidin-4(3H)-one | | |
| | F | C34 | b: 4-(bromomethyl)tetrahydropyran |
| C66 | 7-(3,4-dichlorobenzoyl)-3-[1-(methanesulfonyl)piperidin-4-yl]-2-{[(4-methoxy- | | |
| | phenyl)methyl]amino}-5,6,7,8-tetrahydropyrido[3,4-d]pyrimidin-4(3H)-one | | |
| | K | I28 | a: 4-amino-1-methanesulfonylpiperidine; b: 3,4- |
| | | | dichlorobenzoyl chloride; c: 4-methoxybenzylamine |
| C67 | 7-(3,4-dichlorobenzoyl)-2-{[(4-methoxyphenyl)methyl]amino}-3-(2- | | |
| | methylpropyl)-5,6,7,8-tetrahydropyrido[3,4-d]pyrimidin-4(3H)-one | | |
| | G | I13 | a: N-isobutylthiourea; b: 4-methoxybenzylamine |
| C68 | 7-(3,4-dichlorobenzoyl)-2-{[(4-methoxyphenyl)methyl]amino}-3-(pyridin-3-yl)- | | |
| | 5,6,7,8-tetrahydropyrido[3,4-d]pyrimidin-4(3H)-one | | |
| | K | I28 | a: 3-aminopyridine; b: 3,4-dichlorobenzoyl chloride; c: |
| | | | 4-methoxybenzylamine |
| C69 | 7-(4-bromo-3-fluorobenzoyl)-2-{[(4-methoxyphenyl)methyl]amino}-5,6,7,8- | | |
| | tetrahydropyrido[3,4-d]pyrimidin-4(3H)-one | | |
| | I | I26 | a: 4-bromo-3-fluorobenzoyl chloride, conditions B |
| C70 | 3-cyclopropyl-7-(3,4-dichlorobenzoyl)-2-{[(4-methoxyphenyl)methyl]amino}- | | |
| | 5,6,7,8-tetrahydropyrido[3,4-d]pyrimidin-4(3H)-one | | |
| | G | I13 | a: N-cyclopropylthiourea; b: 4-methoxybenzylamine |
| C71 | 7-(3,4-dichlorobenzoyl)-2-{[(4-methoxyphenyl)methyl]amino}-3-propyl- | | |
| | 5,6,7,8-tetrahydropyrido[3,4-d]pyrimidin-4(3H)-one | | |
| | G | I13 | a: N-propylthiourea; b: 4-methoxybenzylamine |
| C72 | 7-(3,4-dichlorobenzoyl)-2-{[(4-methoxyphenyl)methyl]amino}-3-(propan-2-yl)- | | |
| | 5,6,7,8-tetrahydropyrido[3,4-d]pyrimidin-4(3H)-one | | |
| | G | I13 | a: isopropylthiourea; b: 4-methoxybenzylamine |
| C73 | 3-cyclopentyl-7-(3,4-dichlorobenzoyl)-2-{[(4-methoxyphenyl)methyl]amino}- | | |
| | 5,6,7,8-tetrahydropyrido[3,4-d]pyrimidin-4(3H)-one | | |
| | G | I13 | a: N-cyclopentylthiourea; b: 4-methoxybenzylamine |
| C74 | ethyl 3-[7-(3,4-dichlorobenzoyl)-2-{[(4-methoxyphenyl)methyl]amino}-4-oxo- | | |
| | 5,6,7,8-tetrahydropyrido[3,4-d]pyrimidin-3(4H)-yl]benzoate | | |
| | L | I30 | a: ethyl 3-aminobenzoate; b: 4-methoxybenzylamine |
| C75 | 3-[7-(3,4-dichlorobenzoyl)-2-{[(4-methoxyphenyl)methyl]amino}-4-oxo- | | |
| | 5,6,7,8-tetrahydropyrido[3,4-d]pyrimidin-3(4H)-yl]benzoic acid | | |
| | M | C74 | |
| C76 | 2-amino-7-(3,4-dichlorobenzoyl)-3-[(4-methoxyphenyl)methyl]-5,6,7,8- | | |
| | tetrahydropyrido[3,4-d]pyrimidin-4(3H)-one | | |
| | J | I27 | a: 3,4-dichlorobenzoyl chloride |
| C77 | 5-[7-(3,4-dichlorobenzoyl)-2-{[(4-methoxyphenyl)methyl]amino}-4-oxo- | | |
| | 5,6,7,8-tetrahydropyrido[3,4-d]pyrimidin-3(4H)-yl]-2-fluorobenzonitrile | | |
| | L | I30 | a: 5-amino-2-fluorobenzonitrile; b: 4- |
| | | | methoxybenzylamine |
| C78 | 7-(3,4-dichlorobenzoyl)-2-{[(4-methoxyphenyl)methyl]amino}-3-(pyridin-2-yl)- | | |
| | 5,6,7,8-tetrahydropyrido[3,4-d]pyrimidin-4(3H)-one | | |
| | L | I30 | a: 2-aminopyridine; b: 4-methoxybenzylamine |
| C79 | 7-(6-chloro-1H-indole-2-carbonyl)-2-{[(4-methoxyphenyl)methyl]amino}- | | |
| | 5,6,7,8-tetrahydropyrido[3,4-d]pyrimidin-4(3H)-one | | |
| | I | I26 | a: 6-chloroindole-2-carboxylic acid, conditions A |
| C80 | 7-(3,4-dibromobenzoyl)-2-{[(4-methoxyphenyl)methyl]amino}-5,6,7,8- | | |
| | tetrahydropyrido[3,4-d]pyrimidin-4(3H)-one | | |
| | I | I26 | a: 3,4-dibromobenzoic acid, condtions A |
| C81 | 7-(1-benzofuran-5-carbonyl)-2-{[(4-methoxyphenyl)methyl]amino}-5,6,7,8- | | |
| | tetrahydropyrido[3,4-d]pyrimidin-4(3H)-one | | |
| | I | I26 | a: 1-benzofuran-5-carboxylic acid, conditions A |
| C82 | 2-chloro-4-[2-{[(4-methoxyphenyl)methyl]amino}-4-oxo-4,5,6,8- | | |
| | tetrahydropyrido[3,4-d]pyrimidine-7(3H)-carbonyl]benzonitrile | | |
| | I | I26 | a: 3-chloro-4-cyanobenzoyl chloride, conditions B |
| C83 | 7-(3,4-dichlorobenzoyl)-3-[1-(methanesulfonyl)piperidin-3-yl]-2-{[(4-methoxy- | | |
| | phenyl)methyl]amino}-5,6,7,8-tetrahydropyrido[3,4-d]pyrimidin-4(3H)-one | | |
| | L, then | I30 | a: 3-amino-1-N-boc-piperidine; b: 4-methoxybenzyl- |
| | N | | amine |
| C84 | 7-(3,4-dichlorobenzoyl)-3-[4-(methanesulfonyl)phenyl]-2-{[(4-methoxy- | | |
| | phenyl)methyl]amino}-5,6,7,8-tetrahydropyrido[3,4-d]pyrimidin-4(3H)-one | | |
| | L | I30 | a: 4-(methylsulfonyl)aniline; b: 4-methoxybenzylamine |
| C85 | 7-(3,4-dichlorobenzoyl)-3-[4-(methanesulfonyl)phenyl]-2-[(propan-2-yl)- | | |
| | amino]-5,6,7,8-tetrahydropyrido[3,4-d]pyrimidin-4(3H)-one | | |
| | L | I30 | a: 4-(methylsulfonyl)aniline, b: isopropylamine |
| C86 | 7-(3,4-dichlorobenzoyl)-3-(2-methoxyphenyl)-2-{[(2S)-1,1,1-trifluoropropan- | | |
| | 2-yl]amino}-5,6,7,8-tetrahydropyrido[3,4-d]pyrimidin-4(3H)-one | | |
| | C | I12 | a: (S)-2-amino-1,1,1-trifluoropropane, conditions B |
| C87 | 7-(3,4-dichlorobenzoyl)-3-[3-(methanesulfonyl)-5-methoxyphenyl]-2-{[(4- | | |
| | methoxyphenyl)methyl]amino}-5,6,7,8-tetrahydropyrido[3,4-d]pyrimidin- | | |
| | 4(3H)-one | | |
| | L | I30 | a: 3-methoxy-5-(methylsulfonyl)aniline; b: 4-methoxy- |
| | | | benzylamine |
| C88 | 7-(3,4-dichlorobenzoyl)-3-[3-(methanesulfonyl)-5-methoxyphenyl]-2- | | |
| | [(propan-2-yl)amino]-5,6,7,8-tetrahydropyrido[3,4-d]pyrimidin-4(3H)-one | | |
| | L | I30 | a: 3-methoxy-5-(methylsulfonyl)aniline; b: |
| | | | isopropylamine |
| C89 | 7-(3,4-dichlorobenzoyl)-3-(prop-2-en-1-yl)-2-[(prop-2-en-1-yl)amino]-5,6,7,8- | | |
| | tetrahydropyrido[3,4-d]pyrimidin-4(3H)-one | | |
| | E | I13 | a: 1,3-diallylguanidine, b: DBU |
| C90 | 7-(3,4-dichlorobenzoyl)-2-{(3-hydroxypropyl)[(4-methoxyphenyl)methyl]- | | |
| | amino}-5,6,7,8-tetrahydropyrido[3,4-d]pyrimidin-4(3H)-one | | |
| | F | I15 | a: 3-([(4-methoxyphenyl)methyl]amino)propan-1-ol |
| C91 | 7-(3,4-dichlorobenzoyl)-2-{(2-hydroxyethyl)[(4-methoxyphenyl)methyl]- | | |
| | amino}-5,6,7,8-tetrahydropyrido[3,4-d]pyrimidin-4(3H)-one | | |
| | F | I15 | a: 2-[(4-methoxybenzyl)amino]ethanol hydrochloride |
| C92 | 7-(3,4-dichlorobenzoyl)-2-{[3-hydroxy-1-(4-methoxyphenyl)propyl]amino}- | | |
| | 5,6,7,8-tetrahydropyrido[3,4-d]pyrimidin-4(3H)-one | | |
| | F | I15 | a: 3-amino-3-(4-methoxyphenyl)propan-1-ol |
| C93 | 7-(3,4-dichlorobenzoyl)-2-{[(4-methoxyphenyl)methyl]amino}-3-(oxan-4-yl)- | | |
| | 5,6,7,8-tetrahydropyrido[3,4-d]pyrimidin-4(3H)-one | | |
| | L | I30 | a: 4-aminotetrahydropyran; b: 4-methoxybenzylamine |
| C94 | 7-(3,4-dichlorobenzoyl)-6-(4-fluorophenyl)-2-{[(4-methoxyphenyl)methyl]- | | |
| | amino}-5,6,7,8-tetrahydropyrido[3,4-d]pyrimidin-4(3H)-one | | |
| | O | I36- | |
| | | I37 | |
| C95 | 2-amino-7-(3,4-dichlorobenzoyl)-3-(2-methoxyphenyl)-6-methyl-5,6,7,8- | | |
| | tetrahydropyrido[3,4-d]pyrimidin-4(3H)-one | | |
| | H | I23 | a: NH₃ (2M in 1,4-dioxane) |
| C96 | 7-(3,4-dichlorobenzoyl)-2-{[(4-methoxyphenyl)methyl]amino}-3-(2- | | |
| | methylpyridin-3-yl)-5,6,7,8-tetrahydropyrido[3,4-d]pyrimidin-4(3H)-one | | |
| | L | I30 | a: 3-amino-2-methylpyridine; b: 4-methoxybenzylamine |
| C97 | 7-(3,4-dichlorobenzoyl)-2-[(2-hydroxyethyl)(propan-2-yl)amino]-5,6,7,8- | | |
| | tetrahydropyrido[3,4-d]pyrimidin-4(3H)-one | | |
| | F | I15 | a: 2-(isopropylamino)ethanol |
| C98 | 7-(3,4-dichlorobenzoyl)-2-[(1,3-dihydroxypropan-2-yl)amino]-5,6,7,8- | | |
| | tetrahydropyrido[3,4-d]pyrimidin-4(3H)-one | | |
| | F | I15 | a: 2-amino-1,3-propanediol |
| C99 | 7-(3,4-dichlorobenzoyl)-3-(2-methylpyridin-3-yl)-2-[(propan-2-yl)amino]- | | |
| | 5,6,7,8-tetrahydropyrido[3,4-d]pyrimidin-4(3H)-one | | |
| | L | I30 | a: 3-amino-2-methylpyridine; b: isopropylamine |
| C100 | 7-(3,4-dichlorobenzoyl)-3-[4-(methanesulfonyl)phenyl]-6-methyl-2-[(propan- | | |
| | 2-yl)amino]-5,6,7,8-tetrahydropyrido[3,4-d]pyrimidin-4(3H)-one | | |
| | H | I23 | a: isopropylamine |
| C100A | (6R)-7-(3,4-dichlorobenzoyl)-3-[4-(methanesulfonyl)phenyl]-6-methyl-2- | | |
| | [(propan-2-yl)amino]-5,6,7,8-tetrahydropyrido[3,4-d]pyrimidin-4(3H)-one - | | |
| | Enantiomer (R) | | |
| C100B | (6S)-7-(3,4-dichlorobenzoyl)-3-[4-(methanesulfonyl)phenyl]-6-methyl-2- | | |
| | [(propan-2-yl)amino]-5,6,7,8-tetrahydropyrido[3,4-d]pyrimidin-4(3H)-one - | | |
| | Enantiomer (S) | | |
| C101 | 7-(3,4-dichlorobenzoyl)-3-(2-methoxyphenyl)-2-{[(2R)-1,1,1-trifluoropropan- | | |
| | 2-yl]amino}-5,6,7,8-tetrahydropyrido[3,4-d]pyrimidin-4(3H)-one | | |
| | C | I12 | a: (R)-1,1,1-trifluoro-2-propylamine, conditions B |
| C101a | 7-(3,4-dichlorobenzoyl)-3-(2-methoxyphenyl)-2-{[(2R)-1,1,1-trifluoropropan- | | |
| | 2-yl]amino}-5,6,7,8-tetrahydropyrido[3,4-d]pyrimidin-4(3H)-one - Rotamer | | |
| | (*R) | | |
| C101b | 7-(3,4-dichlorobenzoyl)-3-(2-methoxyphenyl)-2-{[(2R)-1,1,1-trifluoropropan- | | |
| | 2-yl]amino}-5,6,7,8-tetrahydropyrido[3,4-d]pyrimidin-4(3H)-one - Rotamer | | |
| | (*S) | | |
| C102 | 7-(3,4-dichlorobenzoyl)-3-[6-(methanesulfonyl)pyridin-3-yl]-2-{[(4-methoxy- | | |
| | phenyl)methyl]amino}-5,6,7,8-tetrahydropyrido[3,4-d]pyrimidin-4(3H)-one | | |
| | L | I30 | a: 6-(methylsulfonyl)-3-pyridinamine; b: 4- |
| | | | methoxybenzylamine |
| C103 | 2-chloro-5-[2-{[(4-methoxyphenyl)methyl]amino}-4-oxo-4,5,6,8- | | |
| | tetrahydropyrido[3,4-d]pyrimidine-7(3H)-carbonyl]benzonitrile | | |
| | I | I26 | a: 4-chloro-3-cyanobenzoic acid, conditions A |
| C104 | 7-(4-bromo-3-methylbenzoyl)-2-{[(4-methoxyphenyl)methyl]amino}-5,6,7,8- | | |
| | tetrahydropyrido[3,4-d]pyrimidin-4(3H)-one | | |
| | I | I26 | a: 4-bromo-3-methylbenzoic acid, conditions A |
| C105 | 2-bromo-5-[2-{[(4-methoxyphenyl)methyl]amino}-4-oxo-4,5,6,8- | | |
| | tetrahydropyrido[3,4-d]pyrimidine-7(3H)-carbonyl]benzonitrile | | |
| | I | I26 | a: 4-bromo-3-cyanobenzoic acid, conditions A |
| C106 | 7-(4-chloro-3-fluorobenzoyl)-2-{[(4-methoxyphenyl)methyl]amino}-5,6,7,8- | | |
| | tetrahydropyrido[3,4-d]pyrimidin-4(3H)-one | | |
| | I | I26 | a: 4-chloro-3-fluorobenzoyl chloride, conditions B |
| C107 | 7-(3,4-dichlorobenzoyl)-2-{[(4-methoxyphenyl)methyl]amino}-3-(1H-pyrazol- | | |
| | 5-yl)-5,6,7,8-tetrahydropyrido[3,4-d]pyrimidin-4(3H)-one | | |
| | Q, then | I30 | a: 1-(2,4-dimethoxybenzyl)-1H-pyrazol-5-amine; b: 4- |
| | AR | | methoxybenzylamine |
| C108 | 7-(3,4-dichlorobenzoyl)-2-{[(4-methoxyphenyl)methyl]amino}-3-(1-methyl- | | |
| | 1H-pyrazol-5-yl)-5,6,7,8-tetrahydropyrido[3,4-d]pyrimidin-4(3H)-one | | |
| | L | I30 | a: 1-methyl-1H-pyrazol-5-amine; b: 4- |
| | | | methoxybenzylamine |
| C109 | 7-(3,4-dichlorobenzoyl)-6-(4-fluorophenyl)-3-[4-(methanesulfonyl)phenyl]-2- | | |
| | {[(4-methoxyphenyl)methyl]amino}-5,6,7,8-tetrahydropyrido[3,4-d]pyrimidin- | | |
| | 4(3H)-one | | |
| | P | I36- | a: 4-(methylsulfonyl)aniline; b: 4-methoxybenzylamine |
| | | I37 | |
| C109A | (6*S)-7-(3,4-dichlorobenzoyl)-6-(4-fluorophenyl)-3-[4-(methanesulfonyl)- | | |
| | phenyl]-2-{[(4-methoxyphenyl)methyl]amino}-5,6,7,8-tetrahydropyrido[3,4- | | |
| | d]pyrimidin-4(3H)-one - Enantiomer (*S) | | |
| C109B | (6*R)-7-(3,4-dichlorobenzoyl)-6-(4-fluorophenyl)-3-[4-(methanesulfonyl)- | | |
| | phenyl]-2-{[(4-methoxyphenyl)methyl]amino}-5,6,7,8-tetrahydropyrido[3,4- | | |
| | d]pyrimidin-4(3H)-one - Enantiomer (*R) | | |
| C110 | 7-(3,4-dichlorobenzoyl)-6-(4-fluorophenyl)-3-[4-(methanesulfonyl)phenyl]-2- | | |
| | [(propan-2-yl)amino]-5,6,7,8-tetrahydropyrido[3,4-d]pyrimidin-4(3H)-one | | |
| | P | I36- | a: 4-(methylsulfonyl)aniline; b: isopropylamine |
| | | I37 | |
| C110A | (6*S)-7-(3,4-dichlorobenzoyl)-6-(4-fluorophenyl)-3-[4-(methanesulfonyl)- | | |
| | phenyl]-2-[(propan-2-yl)amino]-5,6,7,8-tetrahydropyrido[3,4-d]pyrimidin- | | |
| | 4(3H)-one - Enantiomer (*S) | | |
| C110B | (6*R)-7-(3,4-dichlorobenzoyl)-6-(4-fluorophenyl)-3-[4-(methanesulfonyl)- | | |
| | phenyl]-2-[(propan-2-yl)amino]-5,6,7,8-tetrahydropyrido[3,4-d]pyrimidin- | | |
| | 4(3H)-one - Enantiomer (*R) | | |
| cm | N-{2-[7-(3,4-dichlorobenzoyl)-2-{[(4-methoxyphenyl)methyl]amino}-4-oxo- | | |
| | 5,6,7,8-tetrahydropyrido[3,4-d]pyrimidin-3(4H)-yl]ethyl}methanesulfonamide | | |
| | L | I30 | a: N-(2-aminoethyl)methanesulfonamide; b: 4- |
| | | | methoxybenzylamine |
| C112 | N-{2-[7-(3,4-dichlorobenzoyl)-4-oxo-2-[(propan-2-yl)amino]-5,6,7,8- | | |
| | tetrahydropyrido[3,4-d]pyrimidin-3(4H)-yl]ethyl}methanesulfonamide | | |
| | L | I30 | a: N-(2-aminoethyl)methanesulfonamide; b: |
| | | | isopropylamine |
| C113 | 7-(3,4-dichlorobenzoyl)-2-{[(4-methoxyphenyl)methyl]amino}-3-(4- | | |
| | methylpyridin-3-yl)-5,6,7,8-tetrahydropyrido[3,4-d]pyrimidin-4(3H)-one | | |
| | Q | I30 | a: 3-amino-4-methylpyridine; b: 4-methoxybenzylamine |
| C114 | 7-(3,4-dichlorobenzoyl)-3-(4-methylpyridin-3-yl)-2-[(propan-2-yl)amino]- | | |
| | 5,6,7,8-tetrahydropyrido[3,4-d]pyrimidin-4(3H)-one | | |
| | Q | I30 | a: 3-amino-4-methylpyridine; b: isopropylamine |
| C115 | 7-(4-chlorobenzoyl)-2-{[(4-methoxyphenyl)methyl]amino}-5,6,7,8- | | |
| | tetrahydropyrido[3,4-d]pyrimidin-4(3H)-one | | |
| | I | I26 | a: 4-chlorobenzoyl chloride, conditions B |
| C116 | 7-(4-bromobenzoyl)-2-{[(4-methoxyphenyl)methyl]amino}-5,6,7,8- | | |
| | tetrahydropyrido[3,4-d]pyrimidin-4(3H)-one | | |
| | I | I26 | a: 4-bromobenzoyl chloride, conditions B |
| C117 | 7-(4-iodobenzoyl)-2-{[(4-methoxyphenyl)methyl]amino}-5,6,7,8- | | |
| | tetrahydropyrido[3,4-d]pyrimidin-4(3H)-one | | |
| | I | I26 | a: 4-iodobenzoyl chloride, conditions B |
| C118 | 2-{[(4-methoxyphenyl)methyl]amino}-7-[4-(trifluoromethyl)benzoyl]-5,6,7,8- | | |
| | tetrahydropyrido[3,4-d]pyrimidin-4(3H)-one | | |
| | I | I26 | a: 4-(trifluoromethyl)benzoyl chloride, conditions B |
| C119 | 4-[7-(3,4-dichlorobenzoyl)-2-{[(4-methoxyphenyl)methyl]amino}-4-oxo- | | |
| | 5,6,7,8-tetrahydropyrido[3,4-d]pyrimidin-3(4H)-yl]-1λ⁶-thiane-1,1-dione | | |
| | L | I30 | a: 4-aminotetrahydro-2H-thiopyran 1,1-dioxide; b: 4- |
| | | | methoxybenzylamine |
| C120 | 4-[7-(3,4-dichlorobenzoyl)-4-oxo-2-[(propan-2-yl)amino]-5,6,7,8- | | |
| | tetrahydropyrido[3,4-d]pyrimidin-3(4H)-yl]-1λ⁶-thiane-1,1-dione | | |
| | L | I30 | a: 4-aminotetrahydro-2H-thiopyran 1,1-dioxide; b: |
| | | | isopropylamine |
| C121 | ethyl 4-[7-(3,4-dichlorobenzoyl)-2-{[(4-methoxyphenyl)methyl]amino}-4-oxo- | | |
| | 5,6,7,8-tetrahydropyrido[3,4-d]pyrimidin-3(4H)-yl]benzoate | | |
| | L | I30 | a: ethyl 4-aminobenzoate; b: 4-methoxybenzylamine |
| C122 | 7-(3,4-dichlorobenzoyl)-2-[(propan-2-yl)amino]-3-(pyridin-4-yl)-5,6,7,8- | | |
| | tetrahydropyrido[3,4-d]pyrimidin-4(3H)-one | | |
| | Q | I30 | a: 4-aminopyridine; b: isopropylamine |
| C123 | 7-(3,4-dichlorobenzoyl)-2-{[(4-methoxyphenyl)methyl]amino}-3-(pyridin-4-yl)- | | |
| | 5,6,7,8-tetrahydropyrido[3,4-d]pyrimidin-4(3H)-one | | |
| | Q | I30 | a: 4-aminopyridine; b: 4-methoxybenzylamine |
| C124 | 4-[7-(3,4-dichlorobenzoyl)-2-{[(4-methoxyphenyl)methyl]amino}-4-oxo- | | |
| | 5,6,7,8-tetrahydropyrido[3,4-d]pyrimidin-3(4H)-yl]benzoic acid | | |
| | M | C121 | |
| C125 | 3-(4-bromophenyl)-7-(3,4-dichlorobenzoyl)-2-{[(4-methoxyphenyl)methyl]- | | |
| | amino}-5,6,7,8-tetrahydropyrido[3,4-d]pyrimidin-4(3H)-one | | |
| | L | I30 | a: 4-bromoaniline; b: 4-methoxybenzylamine |
| C126 | 3-(4-acetylphenyl)-7-(3,4-dichlorobenzoyl)-2-{[(4-methoxyphenyl)methyl]- | | |
| | amino}-5,6,7,8-tetrahydropyrido[3,4-d]pyrimidin-4(3H)-one | | |
| | L | I30 | a: 4-aminoacetophenone; b: 4-methoxybenzylamine |
| C127 | 3-(4-acetylphenyl)-7-(3,4-dichlorobenzoyl)-2-[(propan-2-yl)amino]-5,6,7,8- | | |
| | tetrahydropyrido[3,4-d]pyrimidin-4(3H)-one | | |
| | L | I30 | a: 4′-aminoacetophenone; b: isopropylamine |
| C128 | 7-(3,4-dichlorobenzoyl)-2-{[(4-methoxyphenyl)methyl]amino}-3-[4- | | |
| | (morpholin-4-yl)phenyl]-5,6,7,8-tetrahydropyrido[3,4-d]pyrimidin-4(3H)-one | | |
| | Q | I30 | a: 4-morpholinoaniline; b: 4-methoxybenzylamine |
| C129 | 4-[7-(3,4-dichlorobenzoyl)-2-{[(4-methoxyphenyl)methyl]amino}-4-oxo- | | |
| | 5,6,7,8-tetrahydropyrido[3,4-d]pyrimidin-3(4H)-yl]-N-methylbenzamide | | |
| | R | I30 | a: ethyl 4-aminobenzoate; b: 4-methoxybenzylamine; c: |
| | | | methylamine |
| C130 | 7-(3,4-dichlorobenzoyl)-3-[4-(morpholin-4-yl)phenyl]-2-[(propan-2-yl)amino]- | | |
| | 5,6,7,8-tetrahydropyrido[3,4-d]pyrimidin-4(3H)-one | | |
| | Q | I30 | a: 4-morpholinoaniline; b: isopropylamine |
| C131 | 3-(1,3-benzoxazol-6-yl)-7-(3,4-dichlorobenzoyl)-2-{[(4-methoxyphenyl)- | | |
| | methyl]amino}-5,6,7,8-tetrahydropyrido[3,4-d]pyrimidin-4(3H)-one | | |
| | L | I30 | a: 1,3-benzoxazol-6-amine; b: 4-methoxybenzylamine |
| C132 | 7-(1-benzofuran-5-carbonyl)-3-[4-(methanesulfonyl)phenyl]-2-[(propan-2- | | |
| | yl)amino]-5,6,7,8-tetrahydropyrido[3,4-d]pyrimidin-4(3H)-one | | |
| | S | I42 | a: 1-benzofuran-5-carboxylic acid |
| C133 | 7-(3-chloro-1H-indole-2-carbonyl)-3-[4-(methanesulfonyl)phenyl]-2-[(propan- | | |
| | 2-yl)amino]-5,6,7,8-tetrahydropyrido[3,4-d]pyrimidin-4(3H)-one | | |
| | S | I42 | a: 3-chloro-1H-indole-2-carboxylic acid |
| C134 | 7-(3,4-dichlorobenzoyl)-2-[(propan-2-yl)amino]-3-(pyridazin-4-yl)-5,6,7,8- | | |
| | tetrahydropyrido[3,4-d]pyrimidin-4(3H)-one | | |
| | R | I30 | a: 4-aminopyridazine; b: isopropylamine |
| C135 | 7-(3,4-dichlorobenzoyl)-2-{[(4-methoxyphenyl)methyl]amino}-3-(pyridazin-4- | | |
| | yl)-5,6,7,8-tetrahydropyrido[3,4-d]pyrimidin-4(3H)-one | | |
| | R | I30 | a: 4-aminopyridazine; b: 4-methoxybenzylamine |
| C136 | 7-(5-fluoro-1H-indole-2-carbonyl)-3-[4-(methanesulfonyl)phenyl]-2-[(propan- | | |
| | 2-yl)amino]-5,6,7,8-tetrahydropyrido[3,4-d]pyrimidin-4(3H)-one | | |
| | S | I42 | a: 5-fluoroindole-2-carboxylic acid |
| C137 | 7-(6-fluoro-1H-indole-2-carbonyl)-3-[4-(methanesulfonyl)phenyl]-2-[(propan- | | |
| | 2-yl)amino]-5,6,7,8-tetrahydropyrido[3,4-d]pyrimidin-4(3H)-one | | |
| | S | I42 | a: 6-fluoroindole-2-carboxylic acid |
| C138 | 3-[4-(methanesulfonyl)phenyl]-2-[(propan-2-yl)amino]-7-(pyrazolo[1,5- | | |
| | a]pyridine-5-carbonyl)-5,6,7,8-tetrahydropyrido[3,4-d]pyrimidin-4(3H)-one | | |
| | S | I42 | a: pyrazolo[1,5-a]pyridine-5-carboxylic acid |
| C139 | 7-(1-benzofuran-6-carbonyl)-3-[4-(methanesulfonyl)phenyl]-2-[(propan-2- | | |
| | yl)amino]-5,6,7,8-tetrahydropyrido[3,4-d]pyrimidin-4(3H)-one | | |
| | S | I42 | a: benzofuran-6-carboxylic acid |
| C140 | (6S)-7-(3,4-dichlorobenzoyl)-3-[4-(methanesulfonyl)phenyl]-2-{[(4- | | |
| | methoxyphenyl)methyl]amino}-6-methyl-5,6,7,8-tetrahydropyrido[3,4- | | |
| | d]pyrimidin-4(3H)-one | | |
| | T | I45-(S) | a: 4-methoxylbenzylamine |
| C141 | (6S)-7-(3,4-dichlorobenzoyl)-3-[4-(methanesulfonyl)phenyl]-6-methyl-2- | | |
| | [(propan-2-yl)amino]-5,6,7,8-tetrahydropyrido[3,4-d]pyrimidin-4(3H)-one | | |
| | T | I45-(S) | a: isopropylamine |
| C142 | (6R)-7-(3,4-dichlorobenzoyl)-3-[4-(methanesulfonyl)phenyl]-6-methyl-2- | | |
| | [(propan-2-yl)amino]-5,6,7,8-tetrahydropyrido[3,4-d]pyrimidin-4(3H)-one | | |
| | T | I45-(R) | a: isopropylamine |
| C143 | 7-(3,4-dichlorobenzoyl)-2-{[(4-methoxyphenyl)methyl]amino}-3-[4-(1H- | | |
| | pyrazol-1-yl)phenyl]-5,6,7,8-tetrahydropyrido[3,4-d]pyrimidin-4(3H)-one | | |
| | L | I30 | a: 4-(1H-pyrazol-1-yl)aniline; b: 4-methoxybenzylamine |
| C144 | 7-(3,4-dichlorobenzoyl)-2-[(propan-2-yl)amino]-3-[4-(1H-pyrazol-1-yl)phenyl]- | | |
| | 5,6,7,8-tetrahydropyrido[3,4-d]pyrimidin-4(3H)-one | | |
| | L | I30 | a: 4-(1H-pyrazol-1-yl)aniline; b: isopropylamine |
| C145 | (6R)-7-(3,4-dichlorobenzoyl)-3-[4-(methanesulfonyl)phenyl]-2-{[(4- | | |
| | methoxyphenyl)methyl]amino}-6-methyl-5,6,7,8-tetrahydropyrido[3,4- | | |
| | d]pyrimidin-4(3H)-one | | |
| | T | I45-(R) | a: 4-methoxybenzylamine |
| C146 | (6R)-7-(3,4-dichlorobenzoyl)-3-[4-(methanesulfonyl)phenyl]-6-methyl-2- | | |
| | {[(1,3-thiazol-4-yl)methyl]amino}-5,6,7,8-tetrahydropyrido[3,4-d]pyrimidin- | | |
| | 4(3H)-one | | |
| | T | I45-(R) | a: 1,3-thiazol-4-ylmethanamine |
| C147 | 3-[4-(methanesulfonyl)phenyl]-7-(3-methyl-1H-indole-2-carbonyl)-2-[(propan- | | |
| | 2-yl)amino]-5,6,7,8-tetrahydropyrido[3,4-d]pyrimidin-4(3H)-one | | |
| | S | I42 | a: 3-methyl-1H-indole-2-carboxylic acid |
| C148 | 3-(1,3-benzoxazol-4-yl)-7-(3,4-dichlorobenzoyl)-2-{[(4- | | |
| | methoxyphenyl)methyl]amino}-5,6,7,8-tetrahydropyrido[3,4-d]pyrimidin- | | |
| | 4(3H)-one | | |
| | L | I30 | a: 4-amino-1,3-benzoxazole; b: 4-methoxybenzylamine |
| C149 | 4-[7-(3,4-dichlorobenzoyl)-2-{[(4-methoxyphenyl)methyl]amino}-4-oxo- | | |
| | 5,6,7,8-tetrahydropyrido[3,4-d]pyrimidin-3(4H)-yl]benzamide | | |
| | R | I30 | a: ethyl 4-aminobenzoate; b: 4-methoxybenzylamine; c: |
| | | | ammonia |
| C150 | (6R)-7-(3,4-dichlorobenzoyl)-3-[4-(methanesulfonyl)phenyl]-6-methyl-2- | | |
| | {[(pyrimidin-4-yl)methyl]amino}-5,6,7,8-tetrahydropyrido[3,4-d]pyrimidin- | | |
| | 4(3H)-one | | |
| | T | I45-(R) | a: pyrimidin-4-ylmethanamine |
| C151 | 7-(1,3-benzoxazole-6-carbonyl)-3-[4-(methanesulfonyl)phenyl]-2-[(propan-2- | | |
| | yl)amino]-5,6,7,8-tetrahydropyrido[3,4-d]pyrimidin-4(3H)-one | | |
| | S | I42 | a: benzo[d]oxazole-6-carboxylic acid |
| C152 | 7-(1-benzofuran-2-carbonyl)-3-[4-(methanesulfonyl)phenyl]-2-[(propan-2- | | |
| | yl)amino]-5,6,7,8-tetrahydropyrido[3,4-d]pyrimidin-4(3H)-one | | |
| | S | I42 | a: benzofuran-2-carbonyl chloride |
| C153 | ethyl 4-[(6R)-7-(3,4-dichlorobenzoyl)-6-methyl-4-oxo-2-[(propan-2-yl)amino]- | | |
| | 5,6,7,8-tetrahydropyrido[3,4-d]pyrimidin-3(4H)-yl]benzoate | | |
| | U | I43-(R) | a: ethyl 4-aminobenzoate; b: isopropylamine |
| C154 | 7-(3,4-dichlorobenzoyl)-3-[4-(methanesulfonyl)phenyl]-5-methyl-2-[(propan- | | |
| | 2-yl)amino]-5,6,7,8-tetrahydropyrido[3,4-d]pyrimidin-4(3H)-one | | |
| | W | I56 | |
| C154A | (5*R)-7-(3,4-dichlorobenzoyl)-3-[4-(methanesulfonyl)phenyl]-5-methyl-2- | | |
| | [(propan-2-yl)amino]-5,6,7,8-tetrahydropyrido[3,4-d]pyrimidin-4(3H)-one - | | |
| | Enantiomer (*R) | | |
| C154B | (5*S)-7-(3,4-dichlorobenzoyl)-3-[4-(methanesulfonyl)phenyl]-5-methyl-2- | | |
| | [(propan-2-yl)amino]-5,6,7,8-tetrahydropyrido[3,4-d]pyrimidin-4(3H)-one - | | |
| | Enantiomer (*S) | | |
| C155 | 7-(2,3-dihydro-1-benzofuran-5-carbonyl)-3-[4-(methanesulfonyl)phenyl]-2- | | |
| | [(propan-2-yl)amino]-5,6,7,8-tetrahydropyrido[3,4-d]pyrimidin-4(3H)-one | | |
| | S | I42 | a: 2,3-dihydrobenzo[b]furan-5-carboxylic acid |
| C156 | methyl 4-{4-[7-(3,4-dichlorobenzoyl)-2-{[(4-methoxyphenyl)methyl]amino}-4- | | |
| | oxo-5,6,7,8-tetrahydropyrido[3,4-d]pyrimidin-3(4H)-yl]phenyl}morpholine-3- | | |
| | carboxylate | | |
| | L | I30 | a: 4-(4-aminophenyl)morpholine-3-carboxylic acid ethyl |
| | | | ester; b: 4-methoxybenzylamine |
| C157 | 4-[(6R)-7-(3,4-dichlorobenzoyl)-6-methyl-4-oxo-2-[(propan-2-yl)amino]- | | |
| | 5,6,7,8-tetrahydropyrido[3,4-d]pyrimidin-3(4H)-yl]-N-methylbenzamide | | |
| | X | I70 | a: methylamine |
| C158 | 7-(3,4-dichlorobenzoyl)-6-ethyl-3-[4-(methanesulfonyl)phenyl]-2-[(propan-2- | | |
| | yl)amino]-5,6,7,8-tetrahydropyrido[3,4-d]pyrimidin-4(3H)-one | | |
| | Y | I64 | a: 3,4-dichlorobenzoyl chloride |
| C158A | (6*R)-7-(3,4-dichlorobenzoyl)-6-ethyl-3-[4-(methanesulfonyl)phenyl]-2- | | |
| | [(propan-2-yl)amino]-5,6,7,8-tetrahydropyrido[3,4-d]pyrimidin-4(3H)-one - | | |
| | Enantiomer (*R) | | |
| C158B | (6*S)-7-(3,4-dichlorobenzoyl)-6-ethyl-3-[4-(methanesulfonyl)phenyl]-2- | | |
| | [(propan-2-yl)amino]-5,6,7,8-tetrahydropyrido[3,4-d]pyrimidin-4(3H)-one - | | |
| | Enantiomer (*S) | | |
| C159 | 4-{4-[7-(3,4-dichlorobenzoyl)-2-{[(4-methoxyphenyl)methyl]amino}-4-oxo- | | |
| | 5,6,7,8-tetrahydropyrido[3,4-d]pyrimidin-3(4H)-yl]phenyl}morpholine-3- | | |
| | carboxylic acid | | |
| | R | I30 | a: methyl 4-(4-aminophenyl)morpholine-3-carboxylate; |
| | | | b: 4-methoxybenzylamine |
| C160 | 7-(3,4-dichlorobenzoyl)-2-{[(4-methoxyphenyl)methyl]amino}-3-[4-(1,3- | | |
| | oxazol-2-yl)phenyl]-5,6,7,8-tetrahydropyrido[3,4-d]pyrimidin-4(3H)-one | | |
| | L | I30 | a: 4-(1,3-oxazol-2-yl)aniline; b: 4-methoxybenzylamine |
| C161 | tert-butyl (1-{4-[7-(3,4-dichlorobenzoyl)-2-{[(4-methoxyphenyl)methyl]amino}- | | |
| | 4-oxo-5,6,7,8-tetrahydropyrido[3,4-d]pyrimidin-3(4H)-yl]phenyl}pyrrolidin-3- | | |
| | yl)carbamate | | |
| | Z | I67 | a: 3-(tert-butoxycarbonylamino)pyrrolidine |
| C162 | 7-(3,4-dichlorobenzoyl)-3-[4-(1,3-oxazol-2-yl)phenyl]-2-[(propan-2-yl)amino]- | | |
| | 5,6,7,8-tetrahydropyrido[3,4-d]pyrimidin-4(3H)-one | | |
| | L | I30 | a: 4-(1,3-oxazol-2-yl)aniline; b: isopropylamine |
| C163 | 7-(6-chloro-1H-benzimidazole-2-carbonyl)-3-[4-(methanesulfonyl)phenyl]-2- | | |
| | [(propan-2-yl)amino]-5,6,7,8-tetrahydropyrido[3,4-d]pyrimidin-4(3H)-one | | |
| | S | I42 | a: 6-chloro-1H-benzoimidazole-2-carboxylic acid |
| C164 | 7-(1-benzothiophene-5-carbonyl)-3-[4-(methanesulfonyl)phenyl]-2-[(propan- | | |
| | 2-yl)amino]-5,6,7,8-tetrahydropyrido[3,4-d]pyrimidin-4(3H)-one | | |
| | S | I42 | a: 1-benzothiophene-5-carboxylic acid |
| C165 | 7-(5,6-difluoro-1H-indole-2-carbonyl)-3-[4-(methanesulfonyl)phenyl]-2- | | |
| | [(propan-2-yl)amino]-5,6,7,8-tetrahydropyrido[3,4-d]pyrimidin-4(3H)-one | | |
| | S | I42 | a: 5,6-difluoroindole-2-carboxylic acid |
| C166 | (6R)-7-(3,4-dichlorobenzoyl)-6-methyl-2-[(propan-2-yl)amino]-3-[4-(1H- | | |
| | pyrazol-1-yl)phenyl]-5,6,7,8-tetrahydropyrido[3,4-d]pyrimidin-4(3H)-one | | |
| | U | I43-(R) | a: 4-(1H-pyrazol-1-yl)aniline; b: isopropylamine |
| C167 | N-cyclopropyl-4-[(6R)-7-(3,4-dichlorobenzoyl)-6-methyl-4-oxo-2-[(propan-2- | | |
| | yl)amino]-5,6,7,8-tetrahydropyrido[3,4-d]pyrimidin-3(4H)-yl]benzamide | | |
| | X | I70 | a: cyclopropylamine |
| C168 | (6R)-7-(3,4-dichlorobenzoyl)-6-methyl-3-[4-(morpholine-4-carbonyl)phenyl]- | | |
| | 2-[(propan-2-yl)amino]-5,6,7,8-tetrahydropyrido[3,4-d]pyrimidin-4(3H)-one | | |
| | X | I70 | a: morpholine |
| C169 | (6R)-7-(3,4-dichlorobenzoyl)-6-methyl-3-[4-(4-methylpiperazine-1- | | |
| | carbonyl)phenyl]-2-[(propan-2-yl)amino]-5,6,7,8-tetrahydropyrido[3,4- | | |
| | d]pyrimidin-4(3H)-one | | |
| | X | I70 | a: 1-methylpiperazine |
| C170 | 7-(3,4-dichlorobenzoyl)-2-[(propan-2-yl)amino]-3-[4-(1,3-thiazol-4-yl)phenyl]- | | |
| | 5,6,7,8-tetrahydropyrido[3,4-d]pyrimidin-4(3H)-one | | |
| | AA | I68 | a: 4-(tributylstannyl)thiazole, conditions A |
| C171 | 7-(3,4-dichlorobenzoyl)-3-[4-(3,5-difluoropyridin-2-yl)phenyl]-2-[(propan-2- | | |
| | yl)amino]-5,6,7,8-tetrahydropyrido[3,4-d]pyrimidin-4(3H)-one | | |
| | AA | I68 | a: 3,5-difluoro-2-tributylstannylpyridine, conditions A |
| C172 | 7-(3,4-dichlorobenzoyl)-3-[4-(3-methoxypyridin-2-yl)phenyl]-2-[(propan-2- | | |
| | yl)amino]-5,6,7,8-tetrahydropyrido[3,4-d]pyrimidin-4(3H)-one | | |
| | AA | I68 | a: pyridine, 3-methoxy-2-(tributylstannyl), conditions A |
| C173 | 7-(3,4-dichlorobenzoyl)-3-{4-[6-(morpholin-4-yl)pyridin-2-yl]phenyl}-2- | | |
| | [(propan-2-yl)amino]-5,6,7,8-tetrahydropyrido[3,4-d]pyrimidin-4(3H)-one | | |
| | AA | I68 | a: 4-[6-(tributylstannyl)-2-pyridinyl]morpholine, |
| | | | conditions A |
| C174 | 7-(3,4-dichlorobenzoyl)-3-[4-(1-methyl-1H-imidazol-2-yl)phenyl]-2-[(propan- | | |
| | 2-yl)amino]-5,6,7,8-tetrahydropyrido[3,4-d]pyrimidin-4(3H)-one | | |
| | AA | I68 | a: 1-methyl-2-(tri-N-butylstannyl)imidazole, conditions A |
| C175 | 7-(3,4-dichlorobenzoyl)-3-[4-(furan-2-yl)phenyl]-2-[(propan-2-yl)amino]- | | |
| | 5,6,7,8-tetrahydropyrido[3,4-d]pyrimidin-4(3H)-one | | |
| | AA | I68 | a: 2-(tributylstannyl)furan, conditions A |
| C176 | 7-(3,4-dichlorobenzoyl)-2-[(propan-2-yl)amino]-3-[4-(pyrazin-2-yl)phenyl]- | | |
| | 5,6,7,8-tetrahydropyrido[3,4-d]pyrimidin-4(3H)-one | | |
| | AA | I68 | a: 2-(tributylstannyl)pyrazine, conditions A |
| C177 | 7-(3,4-dichlorobenzoyl)-2-[(propan-2-yl)amino]-3-[4-(pyridazin-4-yl)phenyl]- | | |
| | 5,6,7,8-tetrahydropyrido[3,4-d]pyrimidin-4(3H)-one | | |
| | AA | I68 | a: 4-(tributylstannyl)pyridazine, conditions A |
| C178 | 4-[(6R)-7-(3,4-dichlorobenzoyl)-6-methyl-4-oxo-2-[(propan-2-yl)amino]- | | |
| | 5,6,7,8-tetrahydropyrido[3,4-d]pyrimidin-3(4H)-yl]-N-(propan-2-yl)benzamide | | |
| | X | I70 | a: isopropylamine |
| C179 | methyl 4-{4-[7-(3,4-dichlorobenzoyl)-4-oxo-2-[(propan-2-yl)amino]-5,6,7,8- | | |
| | tetrahydropyrido[3,4-d]pyrimidin-3(4H)-yl]phenyl}morpholine-3-carboxylate | | |
| | AA | I68 | a: methyl-3-morpholine carboxylate, conditions B |
| C180 | 7-(3,4-dichlorobenzoyl)-3-{4-[3-(methylamino)pyrrolidin-1-yl]phenyl}-2- | | |
| | [(propan-2-yl)amino]-5,6,7,8-tetrahydropyrido[3,4-d]pyrimidin-4(3H)-one | | |
| | AA, then | I68 | a: 3-(N-tert-butoxycarbonyl-N-methylamino)pyrrolidine, |
| | AR | | conditions B |
| C181 | 7-(4,5-dichlorothiophene-2-carbonyl)-3-[4-(methanesulfonyl)phenyl]-2- | | |
| | [(propan-2-yl)amino]-5,6,7,8-tetrahydropyrido[3,4-d]pyrimidin-4(3H)-one | | |
| | S | I42 | a: 4,5-dichlorothiophene-2-carboxylic acid |
| C182 | 7-(1-benzothiophene-2-carbonyl)-3-[4-(methanesulfonyl)phenyl]-2-[(propan- | | |
| | 2-yl)amino]-5,6,7,8-tetrahydropyrido[3,4-d]pyrimidin-4(3H)-one | | |
| | S | I42 | a: benzo[b]thiophene-2-carboxylic acid |
| C183 | (6R)-3-(4-bromophenyl)-7-(3,4-dichlorobenzoyl)-6-methyl-2-[(propan-2- | | |
| | yl)amino]-5,6,7,8-tetrahydropyrido[3,4-d]pyrimidin-4(3H)-one | | |
| | U | I43-(R) | a: 4-bromoaniline; b: isopropylamine |
| C184 | 7-(2,3-dihydro-1H-indole-2-carbonyl)-3-[4-(methanesulfonyl)phenyl]-2- | | |
| | [(propan-2-yl)amino]-5,6,7,8-tetrahydropyrido[3,4-d]pyrimidin-4(3H)-one | | |
| | S, then | I42 | a: 1-(tert-butoxycarbonyl)-2-indolinecarboxylic acid |
| | AR | | |
| C185 | 7-(3,4-dichlorobenzoyl)-3-(4-{[(oxan-4-yl)methyl]amino}phenyl)-2-[(propan-2- | | |
| | yl)amino]-5,6,7,8-tetrahydropyrido[3,4-d]pyrimidin-4(3H)-one | | |
| | AA | I68 | a: 4-aminoethyltetrahydropyran, conditions B |
| C186 | 7-(3,4-dichlorobenzoyl)-2-[(propan-2-yl)amino]-3-[4-(1H-pyrazol-4-yl)phenyl]- | | |
| | 5,6,7,8-tetrahydropyrido[3,4-d]pyrimidin-4(3H)-one | | |
| | AA | I68 | a: 1-boc-pyrazole-4-boronic acid pinacol ester, |
| | | | conditions C |
| C187 | 7-(3,4-dichlorobenzoyl)-2-[(propan-2-yl)amino]-3-[4-(pyrimidin-2-yl)phenyl]- | | |
| | 5,6,7,8-tetrahydropyrido[3,4-d]pyrimidin-4(3H)-one | | |
| | AA | I68 | a: 2-(tributylstannyl)pyrimidine, conditions A |
| C188 | 7-(3,4-dichlorobenzoyl)-2-[(propan-2-yl)amino]-3-[4-(1,3-thiazol-5-yl)phenyl]- | | |
| | 5,6,7,8-tetrahydropyrido[3,4-d]pyrimidin-4(3H)-one | | |
| | AA | I68 | a: 5-(4,4,5,5-tetrametyl-1,3,2-dioxaborolan-2-yl)thiazole, |
| | | | conditions C |
| C189 | (6R)-7-(3,4-dichlorobenzoyl)-3-[4-(imidazo[1,2-a]pyridin-7-yl)phenyl]-6- | | |
| | methyl-2-[(propan-2-yl)amino]-5,6,7,8-tetrahydropyrido[3,4-d]pyrimidin- | | |
| | 4(3H)-one | | |
| | AC | I73 | a: 7-(4,4,5,5-tetramethyl-1,3,2dioxaborolan-2- |
| | | | yl)imidazo[1,2-a]pyridine, conditions A |
| C190 | (6R)-7-(3,4-dichlorobenzoyl)-6-methyl-3-{4-[(oxetan-3-yl)amino]phenyl}-2- | | |
| | [(propan-2-yl)amino]-5,6,7,8-tetrahydropyrido[3,4-d]pyrimidin-4(3H)-one | | |
| | AC | I73 | a: 3-oxetamine, conditions B |
| C191 | 7-[4-chloro-3-(trifluoromethyl)benzoyl]-3-[4-(methanesulfonyl)phenyl]-2- | | |
| | [(propan-2-yl)amino]-5,6,7,8-tetrahydropyrido[3,4-d]pyrimidin-4(3H)-one | | |
| | S | I42 | a: 4-chloro-3-(trifluoromethyl)benzoic acid |
| C192 | (6R)-7-(3,4-dichlorobenzoyl)-6-methyl-3-[4-(1-methyl-1H-imidazol-2- | | |
| | yl)phenyl]-2-[(propan-2-yl)amino]-5,6,7,8-tetrahydropyrido[3,4-d]pyrimidin- | | |
| | 4(3H)-one | | |
| | AC | I68 | a: 1-methyl-2-(tributylstqnnyl)-1H-imidazole, conditions |
| | | | C |
| C193 | 7-(4-chloro-3-ethylbenzoyl)-3-[4-(methanesulfonyl)phenyl]-2-[(propan-2- | | |
| | yl)amino]-5,6,7,8-tetrahydropyrido[3,4-d]pyrimidin-4(3H)-one | | |
| | S | I42 | a: 4-chloro-3-ethylbenzoic acid |
| C194 | 7-[3-chloro-4-(trifluoromethyl)benzoyl]-3-[4-(methanesulfonyl)phenyl]-2- | | |
| | [(propan-2-yl)amino]-5,6,7,8-tetrahydropyrido[3,4-d]pyrimidin-4(3H)-one | | |
| | S | I42 | a: 3-chloro-4-(trifluoromethyl)benzoic acid |
| C195 | 4-[(6R)-7-(3,4-dichlorobenzoyl)-6-methyl-4-oxo-2-[(propan-2-yl)amino]- | | |
| | 5,6,7,8-tetrahydropyrido[3,4-d]pyrimidin-3(4H)-yl]benzamide | | |
| | AD | I70 | |
| C196 | (6R)-7-(3,4-dichlorobenzoyl)-3-[4-(1H-imidazol-2-yl)phenyl]-6-methyl-2- | | |
| | [(propan-2-yl)amino]-5,6,7,8-tetrahydropyrido[3,4-d]pyrimidin-4(3H)-one | | |
| | AD | I70 | |
| C197 | N-(2-aminoethyl)-4-[(6R)-7-(3,4-dichlorobenzoyl)-6-methyl-4-oxo-2-[(propan- | | |
| | 2-yl)amino]-5,6,7,8-tetrahydropyrido[3,4-d]pyrimidin-3(4H)-yl]benzamide | | |
| | AE | I70 | |
| C198 | (6R)-7-(3,4-dichlorobenzoyl)-3-[4-(methanesulfonyl)phenyl]-6-methyl-2-{[1- | | |
| | (pyridin-4-yl)cyclopropyl]amino}-5,6,7,8-tetrahydropyrido[3,4-d]pyrimidin- | | |
| | 4(3H)-one | | |
| | T | I45-(R) | a: 1-(4-pyridinyl)-cyclopropylamine dihydrochloride |
| C199 | (6R)-2-[(1-cyclopropylethyl)amino]-7-(3,4-dichlorobenzoyl)-3-[4-(methane- | | |
| | sulfonyl)phenyl]-6-methyl-5,6,7,8-tetrahydropyrido[3,4-d]pyrimidin-4(3H)-one | | |
| | T | I45-(R) | a: 1-cyclopropylethanamine |
| C200 | (6R)-7-(3,4-dichlorobenzoyl)-3-{4-[(3S)-3-hydroxypyrrolidin-1-yl]phenyl}-6- | | |
| | methyl-2-[(propan-2-yl)amino]-5,6,7,8-tetrahydropyrido[3,4-d]pyrimidin- | | |
| | 4(3H)-one | | |
| | AC | I73 | a: (R)-3-hydroxypyrrolidine, conditions D |
| C201 | (6R)-7-(3,4-dichlorobenzoyl)-3-{4-[(3R)-3-hydroxypyrrolidin-1-yl]phenyl}-6- | | |
| | methyl-2-[(propan-2-yl)amino]-5,6,7,8-tetrahydropyrido[3,4-d]pyrimidin- | | |
| | 4(3H)-one | | |
| | AC | I73 | a: (S)-3-hydroxypyrrolidine, conditions D |
| C202 | (6R)-7-(3,4-dichlorobenzoyl)-6-methyl-3-[4-(3-methyl-4-oxoimidazolidin-1- | | |
| | yl)phenyl]-2-[(propan-2-yl)amino]-5,6,7,8-tetrahydropyrido[3,4-d]pyrimidin- | | |
| | 4(3H)-one | | |
| | AC | I73 | a: 3-methylimidazolidin-4-one hydrochloride, conditions |
| | | | D |
| C203 | 4-[(6R)-7-(3,4-dichlorobenzoyl)-6-methyl-4-oxo-2-[(propan-2-yl)amino]- | | |
| | 5,6,7,8-tetrahydropyrido[3,4-d]pyrimidin-3(4H)-yl]-N-[2-(morpholin-4- | | |
| | yl)ethyl]benzamide | | |
| | X | I70 | a: 4-(2-aminoethyl)morpholine |
| C204 | 4-[(6R)-7-(3,4-dichlorobenzoyl)-6-methyl-4-oxo-2-[(propan-2-yl)amino]- | | |
| | 5,6,7,8-tetrahydropyrido[3,4-d]pyrimidin-3(4H)-yl]-N-(2-hydroxyethyl)- | | |
| | benzamide | | |
| | X | I70 | a: ethanolamine |
| C205 | 4-[(6R)-7-(3,4-dichlorobenzoyl)-6-methyl-4-oxo-2-[(propan-2-yl)amino]- | | |
| | 5,6,7,8-tetrahydropyrido[3,4-d]pyrimidin-3(4H)-yl]-N-(2-methoxyethyl)- | | |
| | benzamide | | |
| | X | I70 | a: 2-methoxyethylamine |
| C206 | (6R)-7-(3,4-dichlorobenzoyl)-6-methyl-3-(2-oxo-2,3-dihydro-1H-indol-5-yl)-2- | | |
| | [(propan-2-yl)amino]-5,6,7,8-tetrahydropyrido[3,4-d]pyrimidin-4(3H)-one | | |
| | U | I43-(R) | a: 5-aminooxindole; b: isopropylamine |
| C207 | (6R)-7-(3,4-dichlorobenzoyl)-3-[4-(methanesulfonyl)phenyl]-6-methyl-2-{[2- | | |
| | (1-methyl-1H-pyrazol-4-yl)propan-2-yl]amino}-5,6,7,8-tetrahydropyrido[3,4- | | |
| | d]pyrimidin-4(3H)-one | | |
| | T | I45-(R) | a: 2-(1-methyl-1H-pyrazol-4-yl)propan-2-amine |
| C208 | 4-[(6R)-7-(3,4-dichlorobenzoyl)-6-methyl-4-oxo-2-[(propan-2-yl)amino]- | | |
| | 5,6,7,8-tetrahydropyrido[3,4-d]pyrimidin-3(4H)-yl]-N-[2-(4-methylpiperazin-1- | | |
| | yl)ethyl]benzamide | | |
| | X | I70 | a: 2-(4-methylpiperazin-1-yl)ethanamine |
| C209 | 4-[(6R)-7-(3,4-dichlorobenzoyl)-6-methyl-4-oxo-2-[(propan-2-yl)amino]- | | |
| | 5,6,7,8-tetrahydropyrido[3,4-d]pyrimidin-3(4H)-yl]-N-(oxetan-3-yl)benzamide | | |
| | X | I70 | a: 3-oxetanamine |
| C210 | 2-chloro-4-[7-(3,4-dichlorobenzoyl)-4-oxo-2-[(propan-2-yl)amino]-5,6,7,8- | | |
| | tetrahydropyrido[3,4-d]pyrimidin-3(4H)-yl]-N-methylbenzamide | | |
| | L | I30 | a: 4-amino-2-chloro-N-methylbenzamide; b: |
| | | | isopropylamine |
| C211 | (6R)-7-(3,4-dichlorobenzoyl)-3-[4-(3,5-dimethyl-1,2-oxazol-4-yl)phenyl]-6- | | |
| | methyl-2-[(propan-2-yl)amino]-5,6,7,8-tetrahydropyrido[3,4-d]pyrimidin- | | |
| | 4(3H)-one | | |
| | AC | I73 | a: 3,5-dimethylisoxazole-4-boronic acid, conditions A |
| C212 | (6R)-7-(3,4-dichlorobenzoyl)-6-methyl-2-[(propan-2-yl)amino]-3-[4-(1,2- | | |
| | thiazol-5-yl)phenyl]-5,6,7,8-tetrahydropyrido[3,4-d]pyrimidin-4(3H)-one | | |
| | AC | I73 | a: 5-(4,4,5,5-tetramethyl-1,3,2-dioxaborolan-2- |
| | | | yl)isothiazole, conditions A |
| C213 | (6R)-7-(3,4-dichlorobenzoyl)-3-[4-(1H-imidazol-5-yl)phenyl]-6-methyl-2- | | |
| | [(propan-2-yl)amino]-5,6,7,8-tetrahydropyrido[3,4-d]pyrimidin-4(3H)-one | | |
| | AC, then | I73 | a: 1-(tetrahydro-2H-pyran-2-yl)-1H-imidazole-5-boronic |
| | AS | | acid pinacol ester, conditions A |
| C214 | 3-{4-[(6R)-7-(3,4-dichlorobenzoyl)-6-methyl-4-oxo-2-[(propan-2-yl)amino]- | | |
| | 5,6,7,8-tetrahydropyrido[3,4-d]pyrimidin-3(4H)-yl]benzamido}-2,2- | | |
| | dimethylpropanoic acid | | |
| | X | I70 | a: 3-amino-2,2-dimethylpropanoic acid |
| C215 | (6R)-7-(3,4-dichlorobenzoyl)-3-{4-[2-(hydroxymethyl)pyrrolidin-1-yl]phenyl}- | | |
| | 6-methyl-2-[(propan-2-yl)amino]-5,6,7,8-tetrahydropyrido[3,4-d]pyrimidin- | | |
| | 4(3H)-one | | |
| | AC | I73 | a: pyrrolidine-2-ylmethanol, conditions E |
| C216 | 4-{4-[(6R)-7-(3,4-dichlorobenzoyl)-6-methyl-4-oxo-2-[(propan-2-yl)amino]- | | |
| | 5,6,7,8-tetrahydropyrido[3,4-d]pyrimidin-3(4H)-yl]phenyl}pyridine-2- | | |
| | carboxylic acid | | |
| | AC | I73 | a: methyl 4-(4,4,5,5-tetramethyl-1,3,2-dioxaborolan-2- |
| | | | yl)picolinate, conditions A |
| C217 | 4-[(6R)-7-(3,4-dichlorobenzoyl)-6-methyl-4-oxo-2-[(propan-2-yl)amino]- | | |
| | 5,6,7,8-tetrahydropyrido[3,4-d]pyrimidin-3(4H)-yl]-N,N-dimethylbenzamide | | |
| | X | I70 | a: dimethylamine |
| C218 | (6R)-7-(3,4-dichlorobenzoyl)-6-methyl-3-[4-(morpholin-4-yl)phenyl]-2- | | |
| | [(propan-2-yl)amino]-5,6,7,8-tetrahydropyrido[3,4-d]pyrimidin-4(3H)-one | | |
| | U | I43-(R) | a: 4-morpholinoaniline; b: isopropylamine |
| C219 | 4-[7-(3,4-dichlorobenzoyl)-4-oxo-2-[(propan-2-yl)amino]-5,6,7,8- | | |
| | tetrahydropyrido[3,4-d]pyrimidin-3(4H)-yl]-2-fluoro-N-methylbenzamide | | |
| | L | I30 | a: 4-amino-2-fluoro-N-methylbenzamide; b: |
| | | | isopropylamine |
| C220 | 4-[7-(3,4-dichlorobenzoyl)-2-{[(4-methoxyphenyl)methyl]amino}-4-oxo- | | |
| | 5,6,7,8-tetrahydropyrido[3,4-d]pyrimidin-3(4H)-yl]-2-fluoro-N-methyl- | | |
| | benzamide | | |
| | L | I30 | a: 4-amino-2-fluoro-N-methylbenzamide; b: 4- |
| | | | methoxybenzylamine |
| C221 | 7-(4-chloro-3-methylbenzoyl)-3-[4-(methanesulfonyl)phenyl]-2-[(propan-2- | | |
| | yl)amino]-5,6,7,8-tetrahydropyrido[3,4-d]pyrimidin-4(3H)-one | | |
| | S | I42 | a: 4-chloro-3-methylbenzoyl cloride |
| C222 | 7-[4-chloro-3-(propan-2-yl)benzoyl]-3-[4-(methanesulfonyl)phenyl]-2- | | |
| | [(propan-2-yl)amino]-5,6,7,8-tetrahydropyrido[3,4-d]pyrimidin-4(3H)-one | | |
| | S | I42 | a: 4-chloro-3-isopropylbenzoic acid |
| C223 | 7-(5,6-dichloropyridine-2-carbonyl)-3-[4-(methanesulfonyl)phenyl]-2- | | |
| | [(propan-2-yl)amino]-5,6,7,8-tetrahydropyrido[3,4-d]pyrimidin-4(3H)-one | | |
| | S | I42 | a: 5,6-dichloropicolinic acid |
| C224 | 3-[4-(methanesulfonyl)phenyl]-7-(3-methyl-1-benzofuran-5-carbonyl)-2- | | |
| | [(propan-2-yl)amino]-5,6,7,8-tetrahydropyrido[3,4-d]pyrimidin-4(3H)-one | | |
| | S | I42 | a: 3-methyl-1-benzofuran-5-carboxylic acid |
| C225 | 7-(4-chloro-3-cyclopropylbenzoyl)-3-[4-(methanesulfonyl)phenyl]-2-[(propan- | | |
| | 2-yl)amino]-5,6,7,8-tetrahydropyrido[3,4-d]pyrimidin-4(3H)-one | | |
| | S, then | I42 | a: 3-bromo-4-chlorobenzoic acid |
| | AF | | |
| C226 | methyl 5-(6R)-7-(3,4-dichlorobenzoyl)-6-methyl-4-oxo-2-[(propan-2- | | |
| | yl)amino]-5,6,7,8-tetrahydropyrido[3,4-d]pyrimidin-3(4H)-yl]pyridine-2- | | |
| | carboxylate | | |
| | U | I43-(R) | a: methyl 5-aminopyridine-2-carboxylate; b: |
| | | | isopropylamine |
| C227 | 5-[(6R)-7-(3,4-dichlorobenzoyl)-6-methyl-4-oxo-2-[(propan-2-yl)amino]- | | |
| | 5,6,7,8-tetrahydropyrido[3,4-d]pyrimidin-3(4H)-yl]-N-(propan-2-yl)pyridine-2- | | |
| | carboxamide | | |
| | U | I43-(R) | a: methyl 5-aminopyridine-2-carboxylate; b: |
| | | | isopropylamine |
| C228 | (6R)-3-(6-chloropyridin-3-yl)-7-(3,4-dichlorobenzoyl)-6-methyl-2-[(propan-2- | | |
| | yl)amino]-5,6,7,8-tetrahydropyrido[3,4-d]pyrimidin-4(3H)-one | | |
| | U | I43-(R) | a: 5-amino-2-chloropyridine; b: isopropylamine |
| C229 | (6R)-7-(3,4-dichlorobenzoyl)-6-methyl-3-(3-oxo-2,3-dihydro-1H-isoindol-5- | | |
| | yl)-2-[(propan-2-yl)amino]-5,6,7,8-tetrahydropyrido[3,4-d]pyrimidin-4(3H)-one | | |
| | U | I43-(R) | a: 6-amino-2,3-dihydro-1H-isoindol-1-one; b: |
| | | | isopropylamine |
| C230 | ethyl 4-[(6R)-2-[(1-cyclopropyl-3-hydroxypropyl)amino]-7-(3,4-dichloro- | | |
| | benzoyl)-6-methyl-4-oxo-5,6,7,8-tetrahydropyrido[3,4-d]pyrimidin-3(4H)- | | |
| | yl]benzoate | | |
| | U | I43-(R) | a: ethyl 4-aminobenzoate; b: 3-amino-3-clopropan-ol |
| | | | hydrochloride |
| C231 | (6R)-7-(3,4-dichlorobenzoyl)-6-methyl-3-(1-oxo-2,3-dihydro-1H-isoindol-5- | | |
| | yl)-2-[(propan-2-yl)amino]-5,6,7,8-tetrahydropyrido[3,4-d]pyrimidin-4(3H)-one | | |
| | U | I43-(R) | a: 5-aminoisoindoline-1-one; b: isopropylamine |
| C232 | 3-[(6R)-7-(3,4-dichlorobenzoyl)-6-methyl-4-oxo-2-[(propan-2-yl)amino]- | | |
| | 5,6,7,8-tetrahydropyrido[3,4-d]pyrimidin-3(4H)-yl]-N-methylbenzamide | | |
| | U | I43-(R) | a: 3-amino-N-methylbenzamide; b: isopropylamine |
| C233 | (6R)-3-(4-bromo-2-chlorophenyl)-7-(3,4-dichlorobenzoyl)-6-methyl-2- | | |
| | [(propan-2-yl)amino]-5,6,7,8-tetrahydropyrido[3,4-d]pyrimidin-4(3H)-one | | |
| | U | I43-(R) | a: 4-bromo-3-chloroaniline; b: isopropylamine |
| C234 | methyl 3-chloro-4-[(6R)-7-(3,4-dichlorobenzoyl)-6-methyl-4-oxo-2-[(propan- | | |
| | 2-yl)amino]-5,6,7,8-tetrahydropyrido[3,4-d]pyrimidin-3(4H)-yl]benzoate | | |
| | U | I43-(R) | a: methyl 4-amino-2-chlorobenzoate; b: isopropylamine |
| C235 | 3-chloro-4-[(6R)-7-(3,4-dichlorobenzoyl)-6-methyl-4-oxo-2-[(propan-2- | | |
| | yl)amino]-5,6,7,8-tetrahydropyrido[3,4-d]pyrimidin-3(4H)-yl]benzoic acid | | |
| | M | C234 | |
| C236 | methyl 3-(6R)-7-(3,4-dichlorobenzoyl)-6-methyl-4-oxo-2-[(propan-2- | | |
| | yl)amino]-5,6,7,8-tetrahydropyrido[3,4-d]pyrimidin-3(4H)-yl]bicyclo[1.1.1]- | | |
| | pentane-1-carboxylate | | |
| | U | I43-(R) | a: methyl 3-aminobicyclo[1.1.1]pentane-1-carboxe |
| | | | hydrochloride; b: isopropylamine |
| C237 | (6R)-7-(3,4-dichlorobenzoyl)-6-methyl-2-[(propan-2-yl)amino]-3-[4- | | |
| | (pyrimidin-2-yl)phenyl]-5,6,7,8-tetrahydropyrido[3,4-d]pyrimidin-4(3H)-one | | |
| | AC | I73 | a: 2-(tributylstannyl)pyrimidine, conditions C |
| C238 | 7-(3,4-dichlorobenzoyl)-2-[(propan-2-yl)amino]-3-[4-(1,3-thiazol-2-yl)phenyl]- | | |
| | 5,6,7,8-tetrahydropyrido[3,4-d]pyrimidin-4(3H)-one | | |
| | AB | I69 | a: 2-bromothiazole |
| C239 | (6R)-7-(3,4-dichlorobenzoyl)-6-methyl-3-[4-(5-methyl-1,3-thiazol-2-yl)- | | |
| | phenyl]-2-[(propan-2-yl)amino]-5,6,7,8-tetrahydropyrido[3,4-d]pyrimidin- | | |
| | 4(3H)-one | | |
| | AG | I74 | a: 3,5-dimethylisoxazole-4-boronic acid, conditions A |
| C240 | (6R)-7-(3,4-dichlorobenzoyl)-6-methyl-2-[(propan-2-yl)amino]-3-[4-(1,3- | | |
| | thiazol-2-yl)phenyl]-5,6,7,8-tetrahydropyrido[3,4-d]pyrimidin-4(3H)-one | | |
| | AG | I74 | a: 2-bromothiazole, conditions A |
| C241 | (6R)-7-(3,4-dichlorobenzoyl)-6-methyl-2-[(propan-2-yl)amino]-3-[4-(1H- | | |
| | pyrazol-3-yl)phenyl]-5,6,7,8-tetrahydropyrido[3,4-d]pyrimidin-4(3H)-one | | |
| | AC | I73 | a: pyrazole-3-boronic acid, conditions A |
| C242 | (6R)-7-(3,4-dichlorobenzoyl)-6-methyl-3-[4-(5-methyl-1,3,4-oxadiazol-2-yl)- | | |
| | phenyl]-2-[(propan-2-yl)amino]-5,6,7,8-tetrahydropyrido[3,4-d]pyrimidin- | | |
| | 4(3H)-one | | |
| | AG | I74 | a: 2-bromo-5-methyl-1,3,4-oxadiazole, conditions B |
| C243 | (6R)-7-(3,4-dichlorobenzoyl)-6-methyl-3-[4-(5-methyl-1H-pyrazol-3-yl)- | | |
| | phenyl]-2-[(propan-2-yl)amino]-5,6,7,8-tetrahydropyrido[3,4-d]pyrimidin- | | |
| | 4(3H)-one | | |
| | AG | I74 | a: 3-bromo-5-methyl-1H-pyrazole, conditions B |
| C244 | (6R)-7-(3,4-dichlorobenzoyl)-6-methyl-3-[4-(2-methyl-1H-imidazol-4-yl)- | | |
| | phenyl]-2-[(propan-2-yl)amino]-5,6,7,8-tetrahydropyrido[3,4-d]pyrimidin- | | |
| | 4(3H)-one | | |
| | AG | I74 | a: 4-bromo-2-methylimidazole, conditions B |
| C245 | (6R)-7-(3,4-dichlorobenzoyl)-6-methyl-3-[4-(5-methyl-1,3,4-thiadiazol-2-yl)- | | |
| | phenyl]-2-[(propan-2-yl)amino]-5,6,7,8-tetrahydropyrido[3,4-d]pyrimidin- | | |
| | 4(3H)-one | | |
| | AG | I74 | a: 2-bromo-5-methyl-1,3,4-thiazole, conditions B |
| C246 | (6R)-7-(3,4-dichlorobenzoyl)-6-methyl-3-[4-(3-methyl-1,2-oxazol-5-yl)- | | |
| | phenyl]-2-[(propan-2-yl)amino]-5,6,7,8-tetrahydropyrido[3,4-d]pyrimidin- | | |
| | 4(3H)-one | | |
| | AG | I74 | a: 5-bromo-3-methyl-1,2-oxazole, conditions B |
| C247 | (6R)-7-(3,4-dichlorobenzoyl)-6-methyl-3-[4-(4-methyl-1,3-thiazol-2-yl)- | | |
| | phenyl]-2-[(propan-2-yl)amino]-5,6,7,8-tetrahydropyrido[3,4-d]pyrimidin- | | |
| | 4(3H)-one | | |
| | AG | I74 | a: 2-bromo-4-methylthiazole, conditions B |
| C248 | (6R)-7-(3,4-dichlorobenzoyl)-6-methyl-3-[4-(5-methyl-1H-imidazol-4-yl)- | | |
| | phenyl]-2-[(propan-2-yl)amino]-5,6,7,8-tetrahydropyrido[3,4-d]pyrimidin- | | |
| | 4(3H)-one | | |
| | AG | I74 | a: 4(5)-bromo5(4)-methyl-imidazole, conditions B |
| C249 | (6R)-3-[4-(5-bromo-1H-1,2,4-triazol-3-yl)phenyl]-7-(3,4-dichlorobenzoyl)-6- | | |
| | methyl-2-[(propan-2-yl)amino]-5,6,7,8-tetrahydropyrido[3,4-d]pyrimidin- | | |
| | 4(3H)-one | | |
| | AG | I74 | a: 3,5-dibromo-1H-1,2,4-triazole, conditions B |
| C250 | (6R)-7-(3,4-dichlorobenzoyl)-6-methyl-3-[4-(4-methyl-1H-imidazol-2-yl)- | | |
| | phenyl]-2-[(propan-2-yl)amino]-5,6,7,8-tetrahydropyrido[3,4-d]pyrimidin- | | |
| | 4(3H)-one | | |
| | AG | I74 | a: 2-bromo-4-methyl-1H-imidazole, conditions B |
| C251 | 4-[(6R)-2-[(bicyclo[1.1.1]pentan-1-yl)amino]-7-(3,4-dichlorobenzoyl)-6- | | |
| | methyl-4-oxo-5,6,7,8-tetrahydropyrido[3,4-d]pyrimidin-3(4H)-yl]-N- | | |
| | methylbenzamide | | |
| | V | I43-(R) | a: bicyclo[1.1.1pentan-1-amine hydrochloride |
| C252 | 4-[(6R)-7-(3,4-dichlorobenzoyl)-6-methyl-2-[(2R)-2-methylpyrrolidin-1-yl]-4- | | |
| | oxo-5,6,7,8-tetrahydropyrido[3,4-d]pyrimidin-3(4H)-yl]-N-methylbenzamide | | |
| | V | I43-(R) | a: (R)-2-methylpyrrolidine |
| C253 | 4-[(6R)-7-(3,4-dichlorobenzoyl)-6-methyl-4-oxo-2-{[(1S)-1-phenylethyl]- | | |
| | amino}-5,6,7,8-tetrahydropyrido[3,4-d]pyrimidin-3(4H)-yl]-N-methyl- | | |
| | benzamide | | |
| | V | I43-(R) | a: (S)-1-phenylethylamine |
| C254 | trans-4-[(6R)-7-(3,4-dichlorobenzoyl)-6-methyl-4-oxo-2-[(propan-2-yl)amino]- | | |
| | 5,6,7,8-tetrahydropyrido[3,4-d]pyrimidin-3(4H)-yl]cyclohexane-1-carboxylic | | |
| | acid | | |
| | U, then | I43-(R) | a: methyl trans-4-aminocyclohexanecarboxylate |
| | M | | hydrochloride; b: idopropylamine |
| C255 | trans-4-[(6R)-7-(3,4-dichlorobenzoyl)-6-methyl-4-oxo-2-[(propan-2-yl)amino]- | | |
| | 5,6,7,8-tetrahydropyrido[3,4-d]pyrimidin-3(4H)-yl]-N-methylcyclohexane-1- | | |
| | carboxamide | | |
| | AH | C254 | |
| C256 | (3*R)-3-cyclopropyl-3-({(6R)-7-(3,4-dichlorobenzoyl)-6-methyl-3-[4- | | |
| | (methylcarbamoyl)phenyl]-4-oxo-3,4,5,6,7,8-hexahydropyrido[3,4- | | |
| | d]pyrimidin-2-yl}amino)propyl methylcarbamate | | |
| | X | C230 | a: methylamine |
| C257 | (3*S)-3-cyclopropyl-3-({(6R)-7-(3,4-dichlorobenzoyl)-6-methyl-3-[4- | | |
| | (methylcarbamoyl)phenyl]-4-oxo-3,4,5,6,7,8-hexahydropyrido[3,4- | | |
| | d]pyrimidin-2-yl}amino)propyl methylcarbamate | | |
| | X | C230 | a: methylamine |
| C258 | 4-[(6R)-2-{[(2R)-butan-2-yl]amino}-7-(3,4-dichlorobenzoyl)-6-methyl-4-oxo- | | |
| | 5,6,7,8-tetrahydropyrido[3,4-d]pyrimidin-3(4H)-yl]-N-methylbenzamide | | |
| | V | I43-(R) | a: (R)-(−)-sec-butylamine |
| C261 | 3-[(6R)-7-(3,4-dichlorobenzoyl)-6-methyl-4-oxo-2-[(propan-2-yl)amino]- | | |
| | 5,6,7,8-tetrahydropyrido[3,4-d]pyrimidin-3(4H)-yl]-N-methylbicyclo- | | |
| | [1.1.1]pentane-1-carboxamide | | |
| | X | C236 | a: methylamine |
| C262 | (6R)-7-(3,4-dichlorobenzoyl)-3-[cis-4-(methanesulfonyl)cyclohexyl]-6-methyl- | | |
| | 2-[(propan-2-yl)amino]-5,6,7,8-tetrahydropyrido[3,4-d]pyrimidin-4(3H)-one | | |
| | AJ | I43-(R) | a: 4-methanesulfonylcyclohexan-1-amine; b: |
| | | | isopropylamine |
| C263 | (6R)-7-(3,4-dichlorobenzoyl)-6-methyl-3-[3-(methylamino)-1,2-benzoxazol-5- | | |
| | yl]-2-[(propan-2-yl)amino]-5,6,7,8-tetrahydropyrido[3,4-d]pyrimidin-4(3H)-one | | |
| | Q, then | I43-(R) | a: tert-butyl (5-amino-1,2-benzoxazol-3- |
| | AS | | yl)methylcarbamate; b: isopropylamine |
| C264 | (6R)-7-(3,4-dichlorobenzoyl)-3-[6-(3,5-dimethyl-1,2-oxazol-4-yl)pyridin-3-yl]- | | |
| | 6-methyl-2-[(propan-2-yl)amino]-5,6,7,8-tetrahydropyrido[3,4-d]pyrimidin- | | |
| | 4(3H)-one | | |
| | AC | C228 | a: 3,5-dimethylisoxazole-4-boronic acid, conditions A |
| C265 | 5-[(6R)-7-(3,4-dichlorobenzoyl)-6-methyl-4-oxo-2-[(propan-2-yl)amino]- | | |
| | 5,6,7,8-tetrahydropyrido[3,4-d]pyrimidin-3(4H)-yl]-N-methylpyridine-2- | | |
| | carboxamide | | |
| | AI | C226 | a: methylamine |
| C266 | 1-{4-[(6R)-7-(3,4-dichlorobenzoyl)-6-methyl-4-oxo-2-[(propan-2-yl)amino]- | | |
| | 5,6,7,8-tetrahydropyrido[3,4-d]pyrimidin-3(4H)-yl]benzamido}cyclopropane- | | |
| | 1-carboxylic acid | | |
| | X | I70 | a: 1-aminocyclopropane-1-carboxylic acid |
| C267 | methyl 1-{4-[(6R)-7-(3,4-dichlorobenzoyl)-6-methyl-4-oxo-2-[(propan-2- | | |
| | yl)amino]-5,6,7,8-tetrahydropyrido[3,4-d]pyrimidin-3(4H)-yl]phenyl}-1H- | | |
| | pyrazole-3-carboxylate | | |
| | U | I43-(R) | a: methyl 1-(4-aminophenyl)-1H-pyrazole-3- |
| | | | carboxylate; b: isopropylamine |
| C268 | (6R)-7-(3,4-dichlorobenzoyl)-3-{4-[5-(difluoromethyl)-1,3,4-oxadiazol-2- | | |
| | yl]phenyl}-6-methyl-2-[(propan-2-yl)amino]-5,6,7,8-tetrahydropyrido[3,4- | | |
| | d]pyrimidin-4(3H)-one | | |
| | AG | I74 | a: 2-bromo-5-(difluoromethyl)-1,3,4-oxadiazole |
| C269 | 4-[(6R)-2-(tert-butylamino)-7-(3,4-dichlorobenzoyl)-6-methyl-4-oxo-5,6,7,8- | | |
| | tetrahydropyrido[3,4-d]pyrimidin-3(4H)-yl]-N-methylbenzamide | | |
| | V | I43-(R) | a: tert-butylamine |
| C270 | 4-[(6R)-7-(3,4-dichlorobenzoyl)-6-methyl-2-[methyl(propan-2-yl)amino]-4- | | |
| | oxo-5,6,7,8-tetrahydropyrido[3,4-d]pyrimidin-3(4H)-yl]-N-methylbenzamide | | |
| | V | I43-(R) | a: N-isopropylethylamine |
| C271 | 4-[(6R)-7-(3,4-dichlorobenzoyl)-6-methyl-2-[(2S)-2-methylpyrrolidin-1-yl]-4- | | |
| | oxo-5,6,7,8-tetrahydropyrido[3,4-d]pyrimidin-3(4H)-yl]-N-methylbenzamide | | |
| | V | I43-(R) | a: (S)-2-methylpyrrolidine |
| C272 | 4-[(6R)-7-(3,4-dichlorobenzoyl)-2-{[(2S)-1-hydroxypropan-2-yl]amino}-6- | | |
| | methyl-4-oxo-5,6,7,8-tetrahydropyrido[3,4-d]pyrimidin-3(4H)-yl]-N- | | |
| | methylbenzamide | | |
| | V | I43-(R) | a: L-alaninol |
| C273 | ethyl cis-4-[(6R)-7-(3,4-dichlorobenzoyl)-6-methyl-4-oxo-2-[(propan-2- | | |
| | yl)amino]-5,6,7,8-tetrahydropyrido[3,4-d]pyrimidin-3(4H)-yl]cyclohexane-1- | | |
| | carboxylate | | |
| | U | I43-(R) | a: cis ethyl-4-aminocyclohexanecarboxylate |
| | | | hydrochloride; b: isopropylamine |
| C274 | 4-[(6R)-7-(3,4-dichlorobenzoyl)-6-methyl-4-oxo-2-[(propan-2-yl)amino]- | | |
| | 5,6,7,8-tetrahydropyrido[3,4-d]pyrimidin-3(4H)-yl]-2-fluoro-N- | | |
| | methylbenzamide | | |
| | U | I43-(R) | a: 4-amino-2-fluoro-N-methylbenzamide; b: |
| | | | isopropylamine |
| C275 | 4-[(6R)-7-(3,4-dichlorobenzoyl)-2-{[(5-methoxy-1H-indol-3-yl)methyl]amino}- | | |
| | 6-methyl-4-oxo-5,6,7,8-tetrahydropyrido[3,4-d]pyrimidin-3(4H)-yl]-N- | | |
| | methylbenzamide | | |
| | V | I43-(R) | a: (5-methoxy-1H-indol-3-yl)methanamine |
| C276 | 4-[(6R)-7-(3,4-dichlorobenzoyl)-2-{[(2S)-4-hydroxybutan-2-yl]amino}-6- | | |
| | methyl-4-oxo-5,6,7,8-tetrahydropyrido[3,4-d]pyrimidin-3(4H)-yl]-N- | | |
| | methylbenzamide | | |
| | V | I43-(R) | a: (S)-3-aminobutan-1-ol |
| C277 | 4-[(6R)-7-(3,4-dichlorobenzoyl)-2-{[(1H-indol-3-yl)methyl]amino}-6-methyl-4- | | |
| | oxo-5,6,7,8-tetrahydropyrido[3,4-d]pyrimidin-3(4H)-yl]-N-methylbenzamide | | |
| | V | I43-(R) | a: (1H-indol-3-yl)methanamine |
| C278 | 4-[(6R)-7-(3,4-dichlorobenzoyl)-6-methyl-2-[(1,2-oxazol-3-yl)amino]-4-oxo- | | |
| | 5,6,7,8-tetrahydropyrido[3,4-d]pyrimidin-3(4H)-yl]-N-methylbenzamide | | |
| | V | I43-(R) | a: 3-aminoisoxazole |
| C279 | (6R)-7-(3,4-dichlorobenzoyl)-6-methyl-2-[(propan-2-yl)amino]-3-{2-[(propan- | | |
| | 2-yl)amino]pyrimidin-5-yl}-5,6,7,8-tetrahydropyrido[3,4-d]pyrimidin-4(3H)-one | | |
| | U | I43-(R) | a: 5-amino-2-chloropyrimidine; b: isopropylamine |
| C280 | cis-4-[(6R)-7-(3,4-dichlorobenzoyl)-6-methyl-4-oxo-2-[(propan-2-yl)amino]- | | |
| | 5,6,7,8-tetrahydropyrido[3,4-d]pyrimidin-3(4H)-yl]-N-methylcyclohexane-1- | | |
| | carboxamide | | |
| | M, then | C273 | |
| | AH | | |
| C281 | 6-[(6R)-7-(3,4-dichlorobenzoyl)-6-methyl-4-oxo-2-[(propan-2-yl)amino]- | | |
| | 5,6,7,8-tetrahydropyrido[3,4-d]pyrimidin-3(4H)-yl]-N-methylpyridazine-3- | | |
| | carboxamide | | |
| | U | I43-(R) | a: 6-amino-N-methylpyridazine-3-carboxamide; b: |
| | | | isopropylamine |
| C282 | 4-[(6R)-2-{[(1*S)-1-cyclopropyl-3-hydroxypropyl]amino}-7-(3,4-dichloro- | | |
| | benzoyl)-6-methyl-4-oxo-5,6,7,8-tetrahydropyrido[3,4-d]pyrimidin-3(4H)-yl]- | | |
| | N-methylbenzamide | | |
| | V | I43-(R) | a: 3-amino-3-cyclopropylpropan-1-ol hydrochloride |
| C283 | 4-[(6R)-2-{[(1*R)-1-cyclopropyl-3-hydroxypropyl]amino}-7-(3,4-dichloro- | | |
| | benzoyl)-6-methyl-4-oxo-5,6,7,8-tetrahydropyrido[3,4-d]pyrimidin-3(4H)-yl]- | | |
| | N-methylbenzamide | | |
| | V | I43-(R) | a: 3-amino-3-cyclopropylpropan-1-ol hydrochloride |
| C284 | 4-[(6R)-7-(3,4-dichlorobenzoyl)-2-{[(1H-indol-5-yl)methyl]amino}-6-methyl-4- | | |
| | oxo-5,6,7,8-tetrahydropyrido[3,4-d]pyrimidin-3(4H)-yl]-N-methylbenzamide | | |
| | V | I43-(R) | a: (1H-indol-5-yl)methanamine |
| C285 | 4-[(6R)-7-(3,4-dichlorobenzoyl)-2-{[(1*R)-1-(2-methoxypyridin-3-yl)ethyl]- | | |
| | amino}-6-methyl-4-oxo-5,6,7,8-tetrahydropyrido[3,4-d]pyrimidin-3(4H)-yl]-N- | | |
| | methylbenzamide - Diastereoisomer (*R) | | |
| | V | I43-(R) | a: 1-(2-methoxypyridin-3-yl)ethanamine |
| C286 | 4-[(rt)-7-(3,4-dichlorobenzoyl)-2-{[(1*S)-1-(2-methoxypyridin-3-yl)ethyl]- | | |
| | amino}-6-methyl-4-oxo-5,6,7,8-tetrahydropyrido[3,4-d]pyrimidin-3(4H)-yl]-N- | | |
| | methylbenzamide - Diastereoisomer (*S) | | |
| | V | I43-(R) | a: 1-(2-methoxypyridin-3-yl)ethanamine |
| C287 | 5-[(6R)-7-(3,4-dichlorobenzoyl)-6-methyl-2-(methylamino)-4-oxo-5,6,7,8- | | |
| | tetrahydropyrido[3,4-d]pyrimidin-3(4H)-yl]-N-methylpyrazine-2-carboxamide | | |
| | U | I43-(R) | a: ethyl-5-aminopyrazine-2-carboxylate; b: methylamine |
| C288 | 5-[(6R)-7-(3,4-dichlorobenzoyl)-6-methyl-4-oxo-2-[(propan-2-yl)amino]- | | |
| | 5,6,7,8-tetrahydropyrido[3,4-d]pyrimidin-3(4H)-yl]-N-methylpyrazine-2- | | |
| | carboxamide | | |
| | AL | I43-(R) | a: ethyl 5-aminopyrazine-2-carboxylate; b:: |
| | | | isopropylamine |
| C289 | 4-[(6R)-7-(3,4-dichlorobenzoyl)-6-methyl-4-oxo-2-[(propan-2-yl)oxy]-5,6,7,8- | | |
| | tetrahydropyrido[3,4-d]pyrimidin-3(4H)-yl]-N-methylbenzamide | | |
| | AK | I43-(R) | a: 4-amino-N-methylbenzamide; b: isopropanol |
| C290 | 4-[(6R)-7-(3,4-dichlorobenzoyl)-6-methyl-2-[methyl(phenyl)amino]-4-oxo- | | |
| | 5,6,7,8-tetrahydropyrido[3,4-d]pyrimidin-3(4H)-yl]-N-methylbenzamide | | |
| | V | I43-(R) | a: N-methylaniline |
| C291 | 4-[(6R)-7-(3,4-dichlorobenzoyl)-6-methyl-2-[methyl(phenyl)amino]-4-oxo- | | |
| | 5,6,7,8-tetrahydropyrido[3,4-d]pyrimidin-3(4H)-yl]-N-methylbenzamide | | |
| | V | I43-(R) | a: N-methylaniline |
| C292 | 3-[(6R)-7-(3,4-dichlorobenzoyl)-6-methyl-4-oxo-2-[(propan-2-yl)amino]- | | |
| | 5,6,7,8-tetrahydropyrido[3,4-d]pyrimidin-3(4H)-yl]-N,1-dimethyl-1H-pyrazole- | | |
| | 5-carboxamide | | |
| | U | I43-(R) | a: 3-amino-N,1-dimethyl-1H-pyrazole-5-carboxamide; b: |
| | | | isopropylamine |
| C293 | 3-chloro-4-[(6R)-7-(3,4-dichlorobenzoyl)-6-methyl-4-oxo-2-[(propan-2- | | |
| | yl)amino]-5,6,7,8-tetrahydropyrido[3,4-d]pyrimidin-3(4H)-yl]-N- | | |
| | methylbenzamide | | |
| | U | I43-(R) | a: 4-amino-3-chloro-N-methylbenzamide; b: |
| | | | isopropylamine |
| C293a | 3-chloro-4-[(6R)-7-(3,4-dichlorobenzoyl)-6-methyl-4-oxo-2-[(propan-2- | | |
| | yl)amino]-5,6,7,8-tetrahydropyrido[3,4-d]pyrimidin-3(4H)-yl]-N- | | |
| | methylbenzamide - Rotamer (*R) | | |
| C293b | 3-chloro-4-[(6R)-7-(3,4-dichlorobenzoyl)-6-methyl-4-oxo-2-[(propan-2- | | |
| | yl)amino]-5,6,7,8-tetrahydropyrido[3,4-d]pyrimidin-3(4H)-yl]-N- | | |
| | methylbenzamide - Rotamer (*S) | | |
| C294 | 4-[(6R)-2-{[(1S)-1-cyclopropylethyl]amino}-7-(3,4-dichlorobenzoyl)-6-methyl- | | |
| | 4-oxo-5,6,7,8-tetrahydropyrido[3,4-d]pyrimidin-3(4H)-yl]-N-methylbenzamide | | |
| | V | I43-(R) | a: 1-cyclopropylethan-1-amine hydrochloride |
| C295 | 4-[(6R)-2-{[(1R)-1-cyclopropylethyl]amino}-7-(3,4-dichlorobenzoyl)-6-methyl- | | |
| | 4-oxo-5,6,7,8-tetrahydropyrido[3,4-d]pyrimidin-3(4H)-yl]-N-methylbenzamide | | |
| | V | I43-(R) | a: 1-cyclopropylethan-1-amine hydrochloride |
| C297 | 4-[(6R)-7-(3,4-dichlorobenzoyl)-6-methyl-2-[(1-methylcyclopropyl)amino]-4- | | |
| | oxo-5,6,7,8-tetrahydropyrido[3,4-d]pyrimidin-3(4H)-yl]-N-methylbenzamide | | |
| | V | I43-(R) | a: 1-methylcyclopropan-1-amine |
| C300a | 4-[(6R)-7-(3,4-dichlorobenzoyl)-6-methyl-4-oxo-2-[(propan-2-yl)amino]- | | |
| | 5,6,7,8-tetrahydropyrido[3,4-d]pyrimidin-3(4H)-yl]-N,3-dimethylbenzamide - | | |
| | Rotamer (*R) | | |
| | U | I43-(R) | a: 4-amino-N,3-dimethylbenzamide; b: isopropylamine |
| C300b | 4-[(6R)-7-(3,4-dichlorobenzoyl)-6-methyl-4-oxo-2-[(propan-2-yl)amino]- | | |
| | 5,6,7,8-tetrahydropyrido[3,4-d]pyrimidin-3(4H)-yl]-N,3-dimethylbenzamide - | | |
| | Rotamer (*S) | | |
| | U | I43-(R) | a: 4-amino-N,3-dimethylbenzamide; b: isopropylamine |
| C302 | 4-[(6R)-2-{[(2S)-but-3-yn-2-yl]amino}-7-(3,4-dichlorobenzoyl)-6-methyl-4- | | |
| | oxo-5,6,7,8-tetrahydropyrido[3,4-d]pyrimidin-3(4H)-yl]-N-methylbenzamide | | |
| | V | I43-(R) | a: (S)-1-methyl-prop-2-ynylamine hydrochloride |
| C303 | 4-[(6R)-7-(3,4-dichlorobenzoyl)-2-(2,2-dimethylazetidin-1-yl)-6-methyl-4-oxo- | | |
| | 5,6,7,8-tetrahydropyrido[3,4-d]pyrimidin-3(4H)-yl]-N-methylbenzamide | | |
| | V | I43-(R) | a: 2,2-dimethylazetidine |
| C304 | 6-[(6R)-7-(3,4-dichlorobenzoyl)-6-methyl-4-oxo-2-[(propan-2-yl)amino]- | | |
| | 5,6,7,8-tetrahydropyrido[3,4-d]pyrimidin-3(4H)-yl]-N-methylpyridine-3- | | |
| | carboxamide | | |
| | AL | I43-(R) | a: methyl 6-aminopyridine-3-carboxylate; b: |
| | | | isopropylamine |
| C305 | (6R)-7-(3,4-dichlorobenzoyl)-6-methyl-3-[3-(methylamino)-1,2-benzoxazol-6- | | |
| | yl]-2-[(propan-2-yl)amino]-5,6,7,8-tetrahydropyrido[3,4-d]pyrimidin-4(3H)-one | | |
| | Q, then | I30 | a: tert-butyl (6-amino-1,2-benzoxazol-3-yl)methyl- |
| | AS | | carbamate; b: isopropylamine |
| C306 | 4-[(6R)-7-(3,4-dichlorobenzoyl)-2-{[(1S)-1-(3-fluorophenyl)ethyl]amino}-6- | | |
| | methyl-4-oxo-5,6,7,8-tetrahydropyrido[3,4-d]pyrimidin-3(4H)-yl]-N- | | |
| | methylbenzamide | | |
| | V | I43-(R) | a: (S)-1-(3-fluorophenyl)ethanamine |
| C307 | 4-[(6R)-7-(3,4-dichlorobenzoyl)-2-(2,2-dimethylpyrrolidin-1-yl)-6-methyl-4- | | |
| | oxo-5,6,7,8-tetrahydropyrido[3,4-d]pyrimidin-3(4H)-yl]-N-methylbenzamide | | |
| | V | I43-(R) | a: 2,2dimethylpyrrolidine |
| C308 | (6R)-7-(3,4-dichlorobenzoyl)-6-methyl-3-[2-methyl-4-(1H-pyrazol-1-yl)- | | |
| | phenyl]-2-[(propan-2-yl)amino]-5,6,7,8-tetrahydropyrido[3,4-d]pyrimidin- | | |
| | 4(3H)-one | | |
| | U | I43-(R) | a: 2-methyl-4-(1H-pyrazol-1-yl)aniline; b: |
| | | | isopropylamine |
| C308a | (6R)-7-(3,4-dichlorobenzoyl)-6-methyl-3-[2-methyl-4-(1H-pyrazol-1-yl)- | | |
| | phenyl]-2-[(propan-2-yl)amino]-5,6,7,8-tetrahydropyrido[3,4-d]pyrimidin- | | |
| | 4(3H)-one - Rotamer (*R) | | |
| C308b | (6R)-7-(3,4-dichlorobenzoyl)-6-methyl-3-[2-methyl-4-(1H-pyrazol-1-yl)- | | |
| | phenyl]-2-[(propan-2-yl)amino]-5,6,7,8-tetrahydropyrido[3,4-d]pyrimidin- | | |
| | 4(3H)-one - Rotamer (*S) | | |
| C311 | 4-[(6R)-7-(3,4-dichlorobenzoyl)-6-methyl-2-[(2*R)-2-methylazetidin-1-yl]-4- | | |
| | oxo-5,6,7,8-tetrahydropyrido[3,4-d]pyrimidin-3(4H)-yl]-N-methylbenzamide | | |
| | V | I43-(R) | a: 2-methylazetidine |
| C312 | 4-[(6R)-7-(3,4-dichlorobenzoyl)-6-methyl-2-[(2*S)-2-methylazetidin-1-yl]-4- | | |
| | oxo-5,6,7,8-tetrahydropyrido[3,4-d]pyrimidin-3(4H)-yl]-N-methylbenzamide | | |
| | V | I43-(R) | a: 2-methylazetidine |
| C313 | (6R)-3-[2-chloro-4-(1-methyl-1H-imidazol-2-yl)phenyl]-7-(3,4-dichloro- | | |
| | benzoyl)-6-methyl-2-[(propan-2-yl)amino]-5,6,7,8-tetrahydropyrido[3,4- | | |
| | d]pyrimidin-4(3H)-one | | |
| | AM | I43-(R) | a: 4-bromo-2-chloroaniline; b: isopropylamine; c: 2- |
| | | | bromo-1-methyl-1H-imidazole |
| C314a | 4-[(6R)-7-(3,4-dichlorobenzoyl)-6-methyl-4-oxo-2-[(propan-2-yl)amino]- | | |
| | 5,6,7,8-tetrahydropyrido[3,4-d]pyrimidin-3(4H)-yl]-3-methoxy-N- | | |
| | methylbenzamide - Rotamer (*R) | | |
| | U | I43-(R) | a: 4-amino-3-methoxy-N-methylbenzamide; b: |
| | | | isopropylamine |
| C314b | 4-[(6R)-7-(3,4-dichlorobenzoyl)-6-methyl-4-oxo-2-[(propan-2-yl)amino]- | | |
| | 5,6,7,8-tetrahydropyrido[3,4-d]pyrimidin-3(4H)-yl]-3-methoxy-N- | | |
| | methylbenzamide - Rotamer (*S) | | |
| | U | I43-(R) | a: 4-amino-3-methoxy-N-methylbenzamide; b: |
| | | | isopropylamine |
| C316 | 4-[(6R)-7-(3,4-dichlorobenzoyl)-2-{[(1S)-1-(2-fluorophenyl)ethyl]amino}-6- | | |
| | methyl-4-oxo-5,6,7,8-tetrahydropyrido[3,4-d]pyrimidin-3(4H)-yl]-N- | | |
| | methylbenzamide | | |
| | V | I43-(R) | a: (S)-1-(2-fluorophenyl)ethylamine |
| C317 | 4-[(6R)-7-(3,4-dichlorobenzoyl)-6-methyl-4-oxo-2-[(propan-2-yl)amino]- | | |
| | 5,6,7,8-tetrahydropyrido[3,4-d]pyrimidin-3(4H)-yl]-2-methoxy-N- | | |
| | methylbenzamide | | |
| | U | I43-(R) | a: 4-amino-2-methoxy-N-methylbenzamide; b: |
| | | | isopropylamine |
| C318 | 4-[(6R)-7-(3,4-dichlorobenzoyl)-2-{[(1*R)-2,2-dimethylcyclopentyl]amino}-6- | | |
| | methyl-4-oxo-5,6,7,8-tetrahydropyrido[3,4-d]pyrimidin-3(4H)-yl]-N- | | |
| | methylbenzamide | | |
| | V | I43-(R) | a: 2,2-dimethylcyclopentan-1-amine |
| C319 | 4-[(6R)-7-(3,4-dichlorobenzoyl)-2-{[(1*S)-2,2-dimethylcyclopentyl]amino}-6- | | |
| | methyl-4-oxo-5,6,7,8-tetrahydropyrido[3,4-d]pyrimidin-3(4H)-yl]-N- | | |
| | methylbenzamide | | |
| | V | I43-(R) | a: 2,2-dimethylcyclopentan-1-amine |
| C320 | 4-[(6R)-7-(3,4-dichlorobenzoyl)-6-methyl-4-oxo-2-{[(1*R)-1-(pyridin-2- | | |
| | yl)ethyl]amino}-5,6,7,8-tetrahydropyrido[3,4-d]pyrimidin-3(4H)-yl]-N- | | |
| | methylbenzamide | | |
| | V | I43-(R) | a: 1-pyridin-2-yl-ethylamine |
| C321 | 4-[(6R)-7-(3,4-dichlorobenzoyl)-6-methyl-4-oxo-2-{[(1*S)-1-(pyridin-2- | | |
| | yl)ethyl]amino}-5,6,7,8-tetrahydropyrido[3,4-d]pyrimidin-3(4H)-yl]-N- | | |
| | methylbenzamide | | |
| | V | I43-(R) | a: 1-pyridin-2-yl-ethylamine |
| C322 | 5-chloro-4-[(6R)-7-(3,4-dichlorobenzoyl)-6-methyl-4-oxo-2-[(propan-2- | | |
| | yl)amino]-5,6,7,8-tetrahydropyrido[3,4-d]pyrimidin-3(4H)-yl]-2-methoxy-N- | | |
| | methylbenzamide | | |
| | U | I43-(R) | a: 4-amino-5-chloro-2-methoxy-N-methylbenzamide; b: |
| | | | isopropylamine |
| C323 | (6R)-7-(3,4-dichlorobenzoyl)-6-methyl-3-(1-oxo-2,3-dihydro-1H-isoindol-4- | | |
| | yl)-2-[(propan-2-yl)amino]-5,6,7,8-tetrahydropyrido[3,4-d]pyrimidin-4(3H)-one | | |
| | U | I43-(R) | a: 4-aminoisoindolin-1-one; b: isopropylamine |
| C324 | (6R)-3-[2-chloro-4-(2-methyl-1H-imidazol-4-yl)phenyl]-7-(3,4-dichloro- | | |
| | benzoyl)-6-methyl-2-[(propan-2-yl)amino]-5,6,7,8-tetrahydropyrido[3,4-d]- | | |
| | pyrimidin-4(3H)-one | | |
| | AM | I43-(R) | a: 4-bromo-2-chloroaniline; b: isopropylamine; b: |
| | | | 4-bromo-2-methyl-1H-imidazole |
| C325 | 4-[(6R)-7-(3,4-dichlorobenzoyl)-2-{[(1H-indol-6-yl)methyl]amino}-6-methyl-4- | | |
| | oxo-5,6,7,8-tetrahydropyrido[3,4-d]pyrimidin-3(4H)-yl]-N-methylbenzamide | | |
| | V | I43-(R) | a: 1H-indole-6-methanamine |
| C326 | 4-[(6R)-7-(3,4-dichlorobenzoyl)-2-{[(2R)-4-hydroxybutan-2-yl]amino}-6- | | |
| | methyl-4-oxo-5,6,7,8-tetrahydropyrido[3,4-d]pyrimidin-3(4H)-yl]-N- | | |
| | methylbenzamide | | |
| | V | I43-(R) | a: (R)-3-aminobutan-1-ol |
| C327 | 5-[(6R)-7-(3,4-dichlorobenzoyl)-6-methyl-2-(methylamino)-4-oxo-5,6,7,8- | | |
| | tetrahydropyrido[3,4-d]pyrimidin-3(4H)-yl]-N-methylpyrimidine-2- | | |
| | carboxamide | | |
| | AN | I43-(R) | a: methyl 5-aminopyridine-2-carboxylate; b: |
| | | | isopropylamine |
| C328 | 5-[(6R)-7-(3,4-dichlorobenzoyl)-6-methyl-4-oxo-2-[(propan-2-yl)amino]- | | |
| | 5,6,7,8-tetrahydropyrido[3,4-d]pyrimidin-3(4H)-yl]-N-methylpyrimidine-2- | | |
| | carboxamide | | |
| | AN | I43-(R) | a: methyl 5-aminopyridine-2-carboxylate; b: |
| | | | isopropylamine |
| C329 | 4-[(6R)-2-(cyclopropylamino)-7-(3,4-dichlorobenzoyl)-6-methyl-4-oxo- | | |
| | 5,6,7,8-tetrahydropyrido[3,4-d]pyrimidin-3(4H)-yl]-N-methylbenzamide | | |
| | V | I43-(R) | a: cyclopropylamine |
| C330 | 4-[(6R)-7-(3,4-dichlorobenzoyl)-2-{[(2R)-1-hydroxypropan-2-yl]amino}-6- | | |
| | methyl-4-oxo-5,6,7,8-tetrahydropyrido[3,4-d]pyrimidin-3(4H)-yl]-N- | | |
| | methylbenzamide | | |
| | V | I43-(R) | a: D-alaninol |
| C331 | 6-[(6R)-7-(3,4-dichlorobenzoyl)-6-methyl-2-(methylamino)-4-oxo-5,6,7,8- | | |
| | tetrahydropyrido[3,4-d]pyrimidin-3(4H)-yl]-N-methylpyridine-3-carboxamide | | |
| | U | I43-(R) | a: methyl 6-aminonicotinate; b: methylamine |
| C332 | 4-[(6R)-2-(2-azaspiro[3.3]heptan-2-yl)-7-(3,4-dichlorobenzoyl)-6-methyl-4- | | |
| | oxo-5,6,7,8-tetrahydropyrido[3,4-d]pyrimidin-3(4H)-yl]-N-methylbenzamide | | |
| | V | I43-(R) | a: 2-azaspiro[3.3]heptane |
| C333 | 4-[(6R)-7-(3,4-dichlorobenzoyl)-2-[(2,2-dimethylcyclopropyl)amino]-6-methyl- | | |
| | 4-oxo-5,6,7,8-tetrahydropyrido[3,4-d]pyrimidin-3(4H)-yl]-N-methylbenzamide | | |
| | V | I43-(R) | a: 2,2-dimethylcyclopropan-1-amine hydrochloride |
| C334 | 4-[(6R)-7-(3,4-dichlorobenzoyl)-2-{[(1S)-2,2-dimethylcyclopropyl]amino}-6- | | |
| | methyl-4-oxo-5,6,7,8-tetrahydropyrido[3,4-d]pyrimidin-3(4H)-yl]-N- | | |
| | methylbenzamide | | |
| | V | I43-(R) | a: (1S)-2,2-dimethylcyclopropan-1-amine hydrochloride |
| C336 | (3R)-3-({(6R)-7-(3,4-dichlorobenzoyl)-6-methyl-3-[4-(methylcarbamoyl)- | | |
| | phenyl]-4-oxo-3,4,5,6,7,8-hexahydropyrido[3,4-d]pyrimidin-2- | | |
| | yl}amino)butanoic acid | | |
| | V | I43-(R) | a: (R)-aminobutyric acid |
| C337 | 4-[(6R)-7-(3,4-dichlorobenzoyl)-6-methyl-4-oxo-2-[(spiro[3.3]heptan-2- | | |
| | yl)amino]-5,6,7,8-tetrahydropyrido[3,4-d]pyrimidin-3(4H)-yl]-N- | | |
| | methylbenzamide | | |
| | V | I43-(R) | a: spiro[3.3]heptane-2-amine hydrochloride |
| C338 | 2-chloro-4-[(6R)-7-(3,4-dichlorobenzoyl)-6-methyl-4-oxo-2-[(propan-2- | | |
| | yl)amino]-5,6,7,8-tetrahydropyrido[3,4-d]pyrimidin-3(4H)-yl]-N- | | |
| | methylbenzamide | | |
| | U | I43-(R) | a: 4-amino-2-chloro-N-methylbenzamide; b: |
| | | | isopropylamine |
| C339 | 4-[(6R)-7-(3,4-dichlorobenzoyl)-6-methyl-4-oxo-2-[(propan-2-yl)amino]- | | |
| | 5,6,7,8-tetrahydropyrido[3,4-d]pyrimidin-3(4H)-yl]-N-methylbenzene-1- | | |
| | sulfonamide | | |
| | AO | I43-(R) | a: 4-amino-N-methylbenzensulfonamide; b: |
| | | | isopropylamine |
| C340a | 4-[(6R)-7-(3,4-dichlorobenzoyl)-6-methyl-4-oxo-2-[(propan-2-yl)amino]- | | |
| | 5,6,7,8-tetrahydropyrido[3,4-d]pyrimidin-3(4H)-yl]-N-methyl-3- | | |
| | (trifluoromethyl)benzamide - Rotamer (*S) | | |
| | U | I43-(R) | a: 4-amino-N-methyl-3-(trifluoromethyl)benzamide; b: |
| | | | isopropylamine |
| C340b | 4-[(6R)-7-(3,4-dichlorobenzoyl)-6-methyl-4-oxo-2-[(propan-2-yl)amino]- | | |
| | 5,6,7,8-tetrahydropyrido[3,4-d]pyrimidin-3(4H)-yl]-N-methyl-3-(trifluoro- | | |
| | methyl)benzamide - Rotamer (*R) | | |
| | U | I43-(R) | a: 4-amino-N-methyl-3-(trifluoromethyl)benzamide; b: |
| | | | isopropylamine |
| C342 | 4-[(6R)-2-{[(*R)-cycopropyl(phenyl)methyl]amino}-7-(3,4-dichlorobenzoyl)-6- | | |
| | methyl-4-oxo-5,6,7,8-tetrahydropyrido[3,4-d]pyrimidin-3(4H)-yl]-N- | | |
| | methylbenzamide | | |
| | V | I43-(R) | a: cyclopropyl(phenyl)methanamine |
| C343 | 4-[(6R)-2-{[(*S)-cycopropyl(phenyl)methyl]amino}-7-(3,4-dichlorobenzoyl)-6- | | |
| | methyl-4-oxo-5,6,7,8-tetrahydropyrido[3,4-d]pyrimidin-3(4H)-yl]-N- | | |
| | methylbenzamide | | |
| | V | I43-(R) | a: cyclopropyl(phenyl)methanamine |
| C344 | 4-[(6R)-7-(3,4-dichlorobenzoyl)-6-methyl-4-oxo-2-[(propan-2-yl)amino]- | | |
| | 5,6,7,8-tetrahydropyrido[3,4-d]pyrimidin-3(4H)-yl]-N-methylpyridine-2- | | |
| | carboxamide | | |
| | AO | I43-(R) | a: 5-amino-N-methylpyridine-2-carboxamide; b: |
| | | | isopropylamine |
| C345 | 4-[(6R)-2-[(bicyclo[1.1.1]pentan-1-yl)(methyl)amino]-7-(3,4-dichlorobenzoyl)- | | |
| | 6-methyl-4-oxo-5,6,7,8-tetrahydropyrido[3,4-d]pyrimidin-3(4H)-yl]-N- | | |
| | methylbenzamide | | |
| | AP | I43-(R) | a: ethyl 4-aminobenzoate; b: bicyclo[1.1.1]pentane-1- |
| | | | amine hydrochloride |
| C346 | 4-[(6R)-7-(3,4-dichlorobenzoyl)-2-{[1-(4-methoxyphenyl)ethyl]amino}-6- | | |
| | methyl-4-oxo-5,6,7,8-tetrahydropyrido[3,4-d]pyrimidin-3(4H)-yl]-N- | | |
| | methylbenzamide | | |
| | V | I43-(R) | a: 1-(4-methoxyphenyl)ethanamine |
| C347 | 4-[(6R)-7-(3,4-dichlorobenzoyl)-6-methyl-4-oxo-2-[(spiro[3.3]heptan-1- | | |
| | yl)amino]-5,6,7,8-tetrahydropyrido[3,4-d]pyrimidin-3(4H)-yl]-N-methyl- | | |
| | benzamide | | |
| | V | I43-(R) | a: spiro[3.3]heptan-1-amine hydrochloride |
| C348 | 4-[(6R)-7-(3,4-dichlorobenzoyl)-6-methyl-4-oxo-2-[(2*R)-2-phenylazetidin-1- | | |
| | yl]-5,6,7,8-tetrahydropyrido[3,4-d]pyrimidin-3(4H)-yl]-N-methylbenzamide | | |
| | V | I43-(R) | a: 2-phenylazetidine |
| C349 | 4-[(6R)-7-(3,4-dichlorobenzoyl)-6-methyl-4-oxo-2-[(2*S)-2-phenylazetidin-1- | | |
| | yl]-5,6,7,8-tetrahydropyrido[3,4-d]pyrimidin-3(4H)-yl]-N-methylbenzamide | | |
| | V | I43-(R) | a: 2-phenylazetidine |
| C350 | 4-[(6R)-7-(3,4-dichlorobenzoyl)-2-[(2*R)-2-ethynylpyrrolidin-1-yl]-6-methyl-4- | | |
| | oxo-5,6,7,8-tetrahydropyrido[3,4-d]pyrimidin-3(4H)-yl]-N-methylbenzamide | | |
| | V | I43-(R) | a: 2-ethynylpyrrolidine hydrochloride |
| C351 | 4-[(6R)-7-(3,4-dichlorobenzoyl)-2-[(2*S)-2-ethynylpyrrolidin-1-yl]-6-methyl-4- | | |
| | oxo-5,6,7,8-tetrahydropyrido[3,4-d]pyrimidin-3(4H)-yl]-N-methylbenzamide | | |
| | V | I43-(R) | a: 2-ethynylpyrrolidine hydrochloride |
| C352 | 4-[(6R)-2-[(2*R)-2-cyclopropylpyrrolidin-1-yl]-7-(3,4-dichlorobenzoyl)-6- | | |
| | methyl-4-oxo-5,6,7,8-tetrahydropyrido[3,4-d]pyrimidin-3(4H)-yl]-N- | | |
| | methylbenzamide | | |
| | V | I43-(R) | a: 2-cyclopropylpyrrolidine hydrochloride |
| C353 | 4-[(6R)-2-[(2*S)-2-cyclopropylpyrrolidin-1-yl]-7-(3,4-dichlorobenzoyl)-6- | | |
| | methyl-4-oxo-5,6,7,8-tetrahydropyrido[3,4-d]pyrimidin-3(4H)-yl]-N- | | |
| | methylbenzamide | | |
| | V | I43-(R) | a: 2-cyclopropylpyrrolidine hydrochloride |
| C354 | 5-[(6R)-7-(3,4-dichlorobenzoyl)-6-methyl-4-oxo-2-[(propan-2-yl)amino]- | | |
| | 5,6,7,8-tetrahydropyrido[3,4-d]pyrimidin-3(4H)-yl]-N,1-dimethyl-1H-pyrazole- | | |
| | 3-carboxamide | | |
| | AQ | I43-(R) | a: methyl 5-amino-1-methyl-1H-pyrazole-3-carboxylate; |
| | | | b: isopropylamine; c: methylamine |
| C355 | 5-[(6R)-7-(3,4-dichlorobenzoyl)-6-methyl-4-oxo-2-[(propan-2-yl)amino]- | | |
| | 5,6,7,8-tetrahydropyrido[3,4-d]pyrimidin-3(4H)-yl]-N-methyl-1H-pyrazole-3- | | |
| | carboxamide | | |
| | AO | I43-(R) | a: 5-amino-N-methyl-1H-pyrazole-3-carboxamide; b: |
| | | | isopropylamine |

### Procedure AT

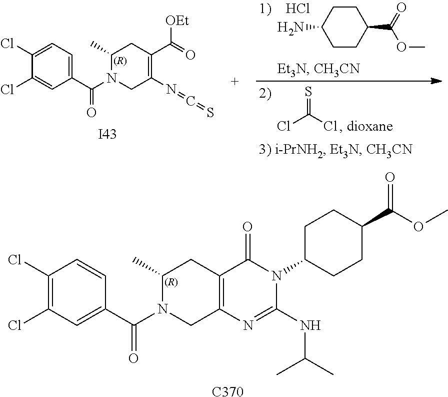

A tube was charged with methyl trans-4-aminocyclohexanecarboxylate hydrochloride (576 mg, 2.97 mmol) in dry CH₃CN (14 mL, 269 mmol). Et₃N (0.86 mL, 6.2 mmol) was added and the mixture was stirred for 5 min. at RT. I43 (i g, 2.50 mmol) was added and the mixture was stirred at RT for 10 min and then at 80° C. for 16 h. The mixture was concentrated in vacuo and the residue partitioned between water and Me-THF. The organic layer dried over MgSO4, filtered and concentrated in vacuo.

The residue was purified by column chromatography (heptane/EtOAc 100/0 to 0/100). The obtained product was triturated in DIPE, filtered off and dried to afford methyl (1R,4r)-4-((R)-7-(3,4-dichlorobenzoyl)-6-methyl-4-oxo-2-thioxo-1,4,5,6,7,8-hexahydropyrido[3,4-d]pyrimidin-3(2H)-yl)cyclohexane-1-carboxylate (1 g, yield 78%) as a white solid.

A vial was charged with methyl (1R,4r)-4-((R)-7-(3,4-dichlorobenzoyl)-6-methyl-4-oxo-2-thioxo-1,4,5,6,7,8-hexahydropyrido[3,4-d]pyrimidin-3(2H)-yl)cyclohexane-1-carboxylate (1 g, 1.95 mmol) in dry 1,4-dioxane (23 mL). Thiophosgene was added and the mixture was stirred at RT for 30 min followed by 10 min. at 100° C. The mixture was concentrated in vacuo. The residue was purified by column chromatography (heptane/EtOAc from 100/0 to 0/100) to afford methyl (1R,4r)-4-((R)-2-chloro-7-(3,4-dichlorobenzoyl)-6-methyl-4-oxo-5,6,7,8-tetrahydropyrido[3,4-d]pyrimidin-3(4H)-yl)cyclohexane-1-carboxylate (834 mg, yield 83%) as a white foam.

A vial was charged with methyl (1R,4r)-4-((R)-2-chloro-7-(3,4-dichlorobenzoyl)-6-methyl-4-oxo-5,6,7,8-tetrahydropyrido[3,4-d]pyrimidin-3(4H)-yl)cyclohexane-1-carboxylate (834 mg, 1.62 mmol), isopropylamine (0.17 mL, 1.95 mmol), Et₃N (0.45 mL, 3.25 mmol) in dry CH₃CN (9 mL). The reaction mixture was stirred at 60° C. for 16h. The mixture was cooled to RT and concentrated in vacuo. The residue was partitioned between water and DCM. The organic layer was separated, dried over MgSO₄ filtered and concentrated in vacuo. The residue was purified on silica column chromatography (heptane/EtOAc from 100/0 to 0/100). The resulting product was triturated in DIPE, filtered off and dried under vacuum to afford C370 (702 mg, yield 81%) as a white solid.

The compounds listed in the table below were obtained in an analogous manner:

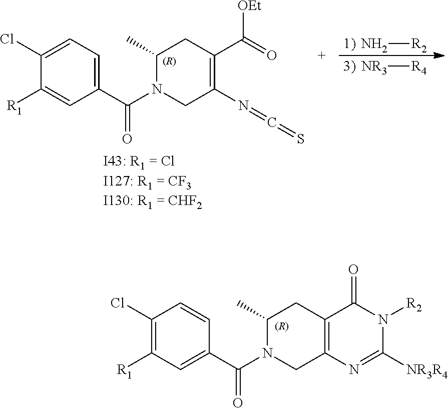

| Cmpd | | | | |
|---|---|---|---|---|
| No | Structure | R | Amine step 1 | Amine step 3 |
| C370 | 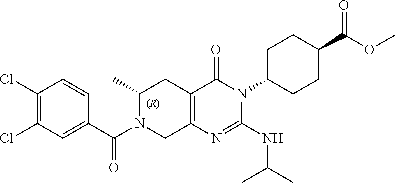 | Cl | 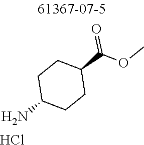 | 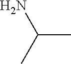 |
| C376 | 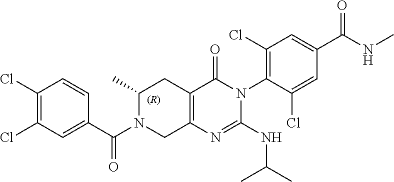 | Cl | 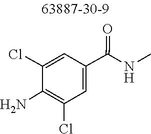 | 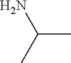 |
| C379 | 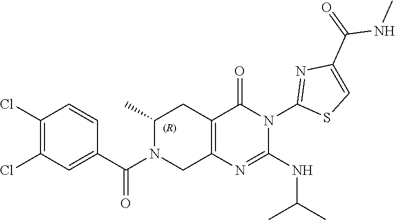 | Cl | 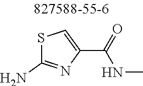 | 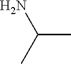 |
| C382^{≠} | 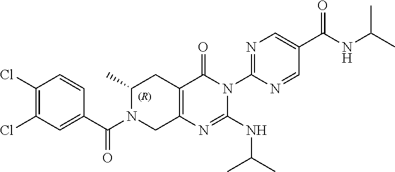 | Cl | 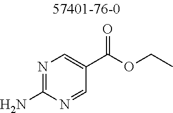 | 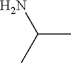 |
| C383 | 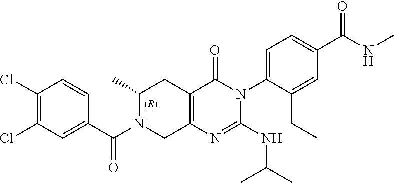 | Cl | 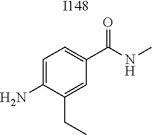 | 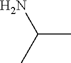 |
| C385 | 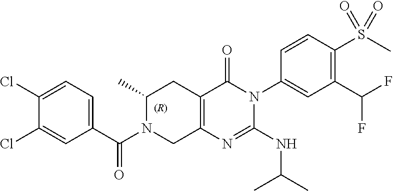 | Cl | 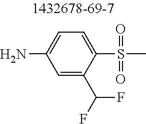 | 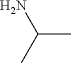 |
| C386 | 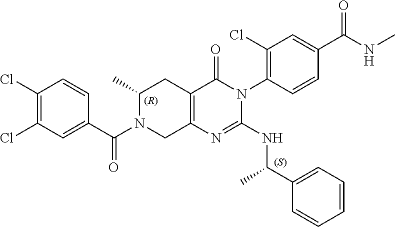 | Cl | 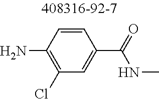 | 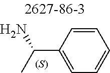 |
| C387 | 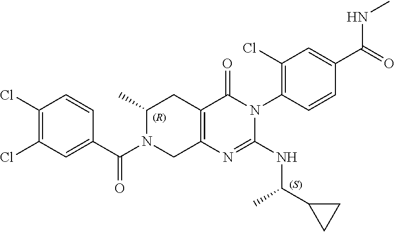 | Cl | 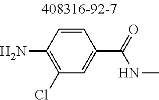 | 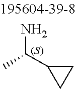 |
| C391 | 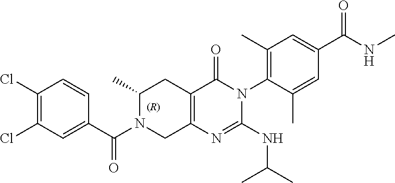 | Cl | 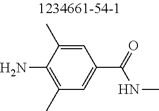 | 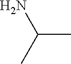 |
| C393 | 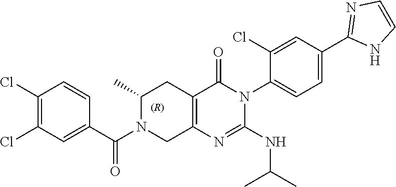 | Cl | 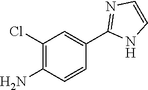 [SMILES: Nc1ccc(-c2ncc[nH]2)cc1Cl] | 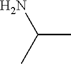 |
| C395 | 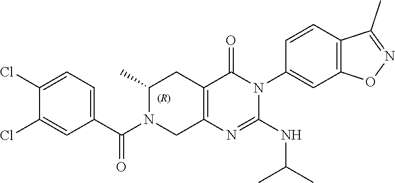 | Cl | 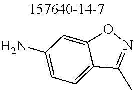 | 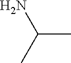 |
| C399 | 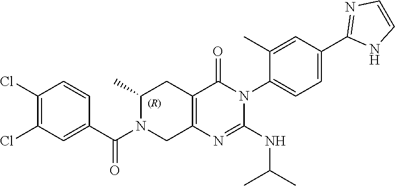 | Cl | 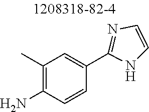 | 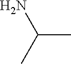 |
| C404^{¥} | 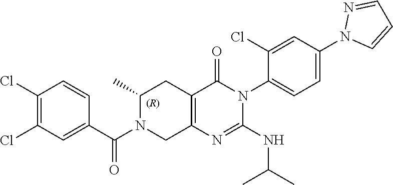 | Cl | 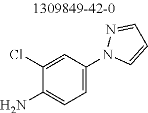 [SMILES: Nc1ccc(-n2cccn2)cc1Cl] | 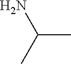 |
| C409 | 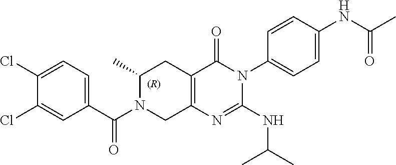 | Cl | 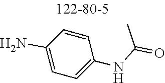 | 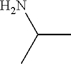 |
| C410 | 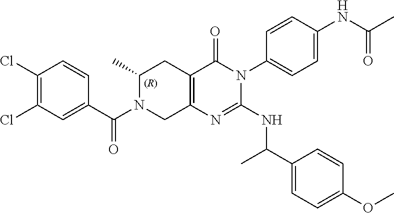 | Cl | 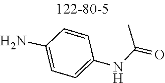 | 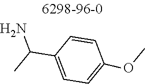 |
| C420 | 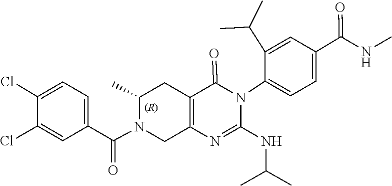 | Cl | 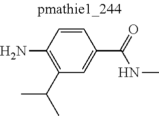 | 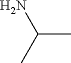 |
| C430 | 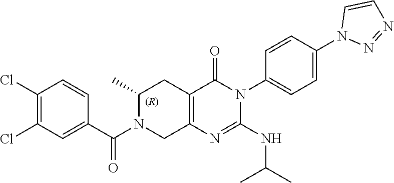 | Cl | 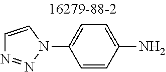 [SMILES: Nc1ccc(-n2ccnn2)cc1] | 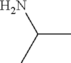 |
| C442 | 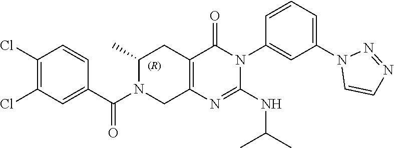 | Cl | 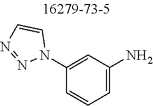 | 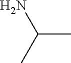 |
| C447 | 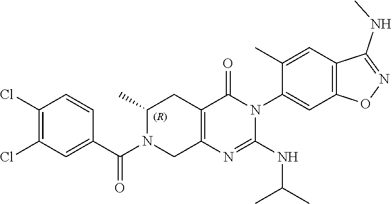 | Cl | 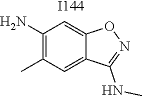 | 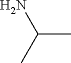 |
| C453 | 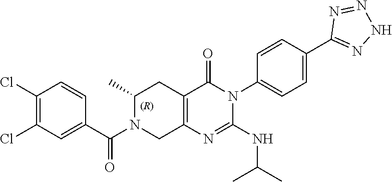 | Cl | 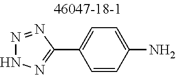 [SMILES: Nc1ccc(-c2nn[nH]n2)cc1] | 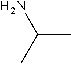 |
| C460 | 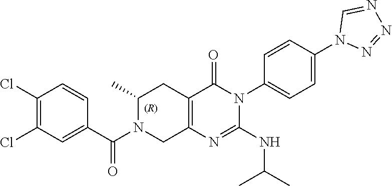 | Cl | 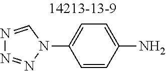 [SMILES: Nc1ccc(-n2cnnn2)cc1] | 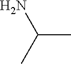 |
| C462 | 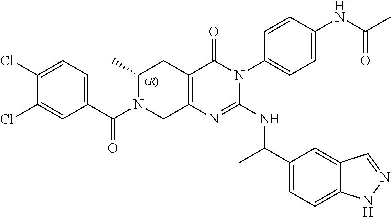 | Cl | 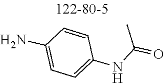 | 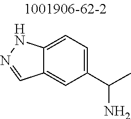 |
| C463 | 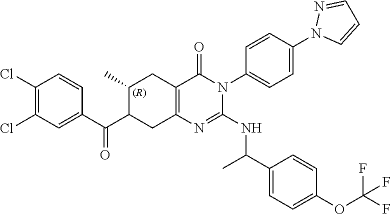 | Cl | 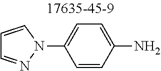 [SMILES: Nc1ccc(-n2cccn2)cc1] | 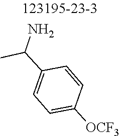 |
| C528^{£} | 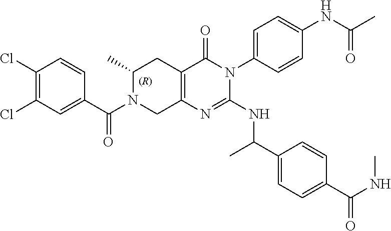 | Cl | 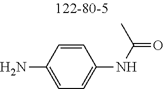 | 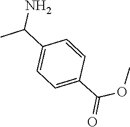 |
| C532 | 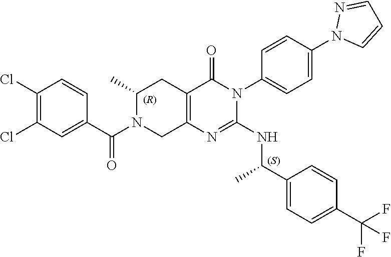 | Cl | 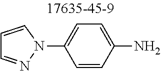 [SMILES: Nc1ccc(-n2cccn2)cc1] | 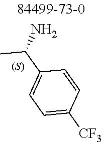 |
| C533 | 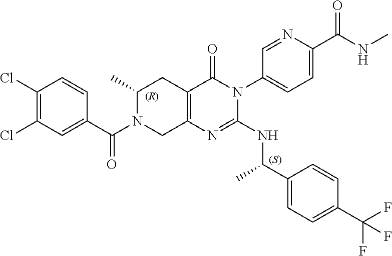 | Cl | 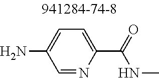 | 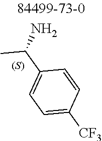 |
| C537^{α} | 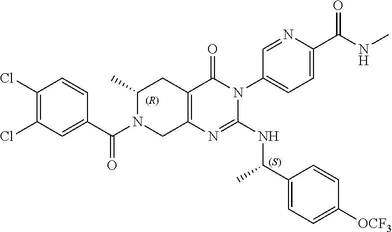 | Cl | 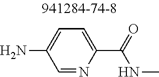 | 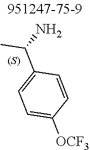 |
| C610 | 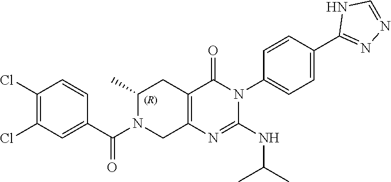 | Cl | 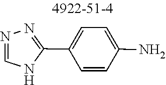 [SMILES: Nc1ccc(-c2nnc[nH]2)cc1] | 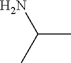 |
| C611 | 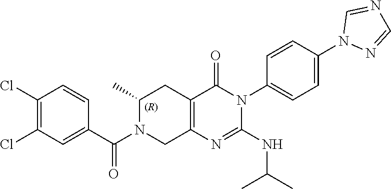 | Cl | 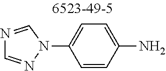 [SMILES: Nc1ccc(-n2cncn2)cc1] | 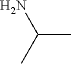 |
| C628 | 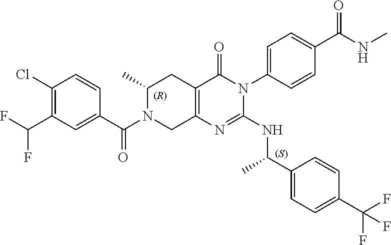 | CHF₂ | 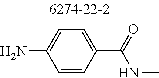 | 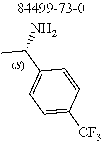 |
| C638 | 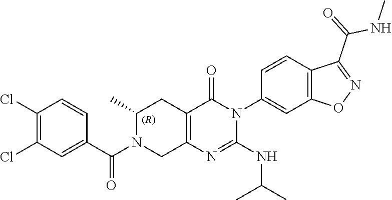 | Cl | 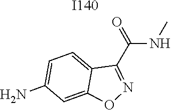 | 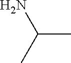 |
| C639 | 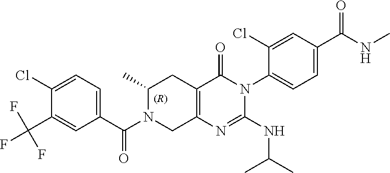 | CF₃ | 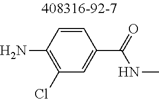 | 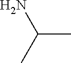 |
| C642 | 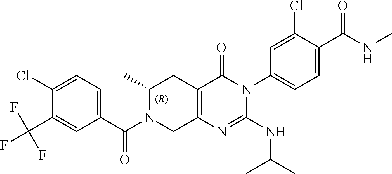 | CF₃ | 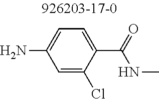 | 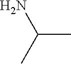 |
| C643 | 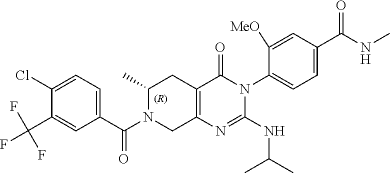 | CF₃ | 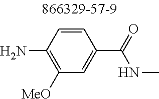 | 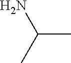 |
| C649 | 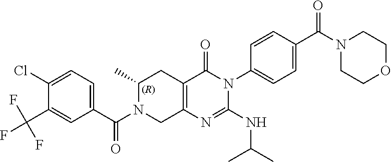 | CF₃ | 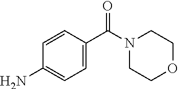 [SMILES: Nc1ccc(C(=O)N2CCOCC2)cc1] | 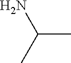 |
| C650 | 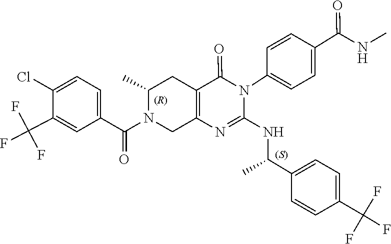 | CF₃ | 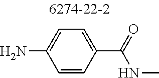 | 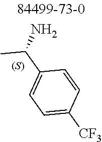 |
| C651 | 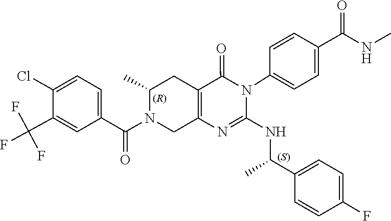 | CF₃ | 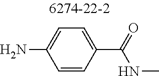 | 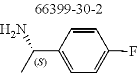 |
| C654 | 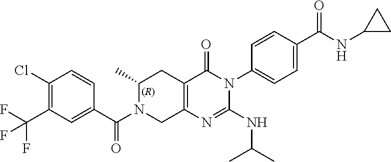 | CF₃ | 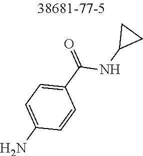 [SMILES: Nc1ccc(C(=O)NC2CC2)cc1] | 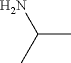 |
| C656 | 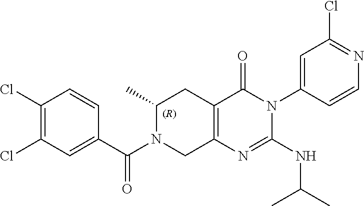 | Cl | 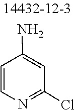 | 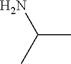 |
| C665 | 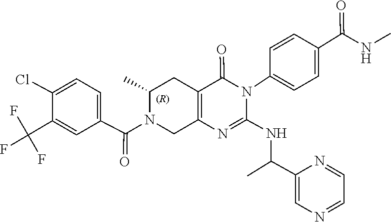 | CF₃ | 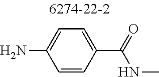 | 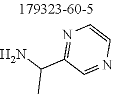 |
| C669 | 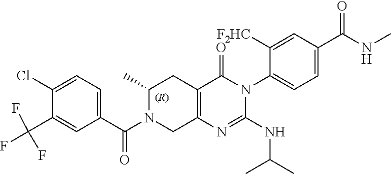 | CF₃ | 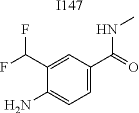 | 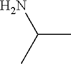 |
| C670 | 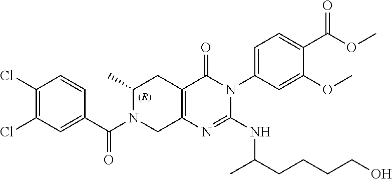 | Cl | 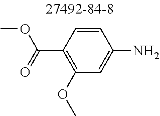 | 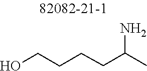 |
| C676 | 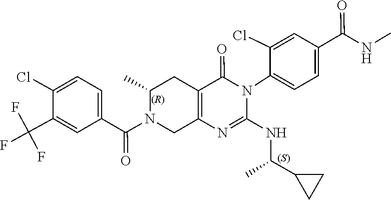 | CF₃ | 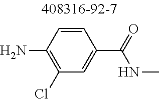 | 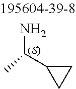 |
| C677 | 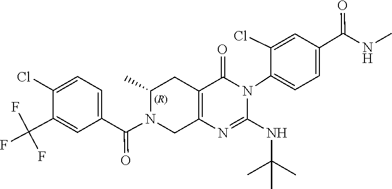 | CF₃ | 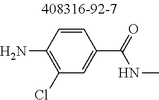 | 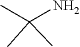 |
| C681 | 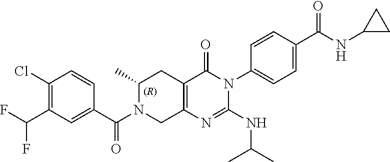 | CHF₂ | 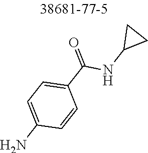 [SMILES: Nc1ccc(C(=O)NC2CC2)cc1] | 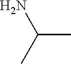 |
| C682 | 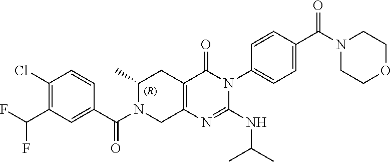 | CHF₂ | 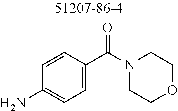 [SMILES: Nc1ccc(C(=O)N2CCOCC2)cc1] | 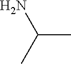 |
| C683 | 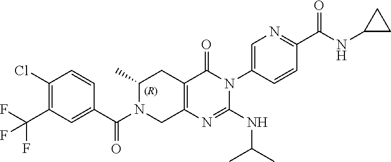 | CF₃ | 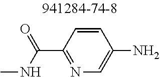 | 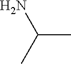 |
| C684 | 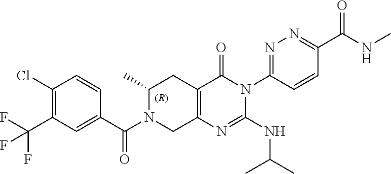 | CF₃ | 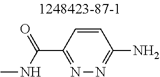 | 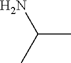 |
| C685 | 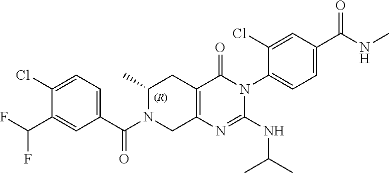 | CHF₂ | 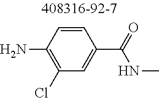 | 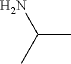 |
| C688 | 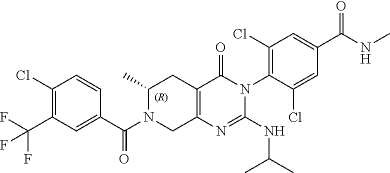 | CF₃ | 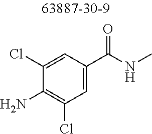 | 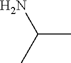 |
| C691 | 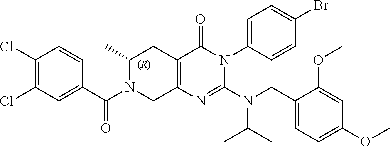 | Cl | 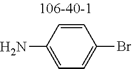 | 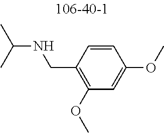 |
| C698 | 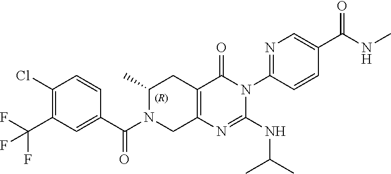 | CF₃ | 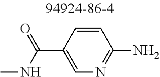 | 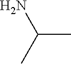 |
| C701 | 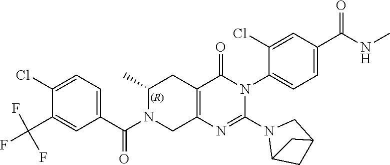 | CF₃ | 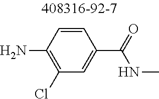 | 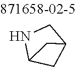 |
| I161 | 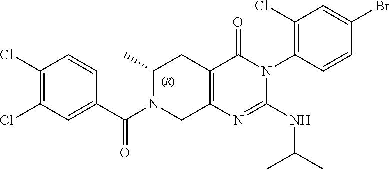 | Cl | 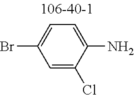 | 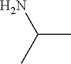 |
| I162 | 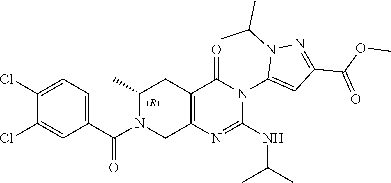 | Cl | 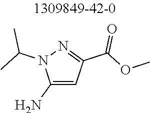 | 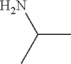 |
| I163 | 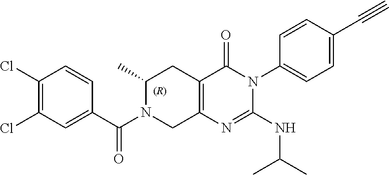 | Cl | 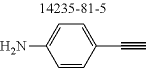 | 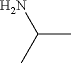 |
| I164 | 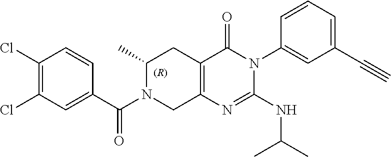 | Cl | 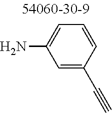 | 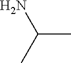 |
| I165 | 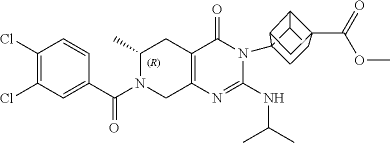 | Cl | 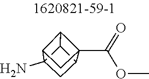 | 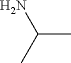 |
| I166 | 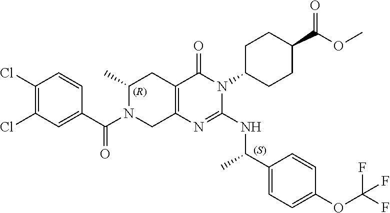 | Cl | 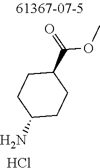 | 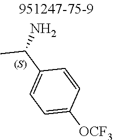 |
| I167 | 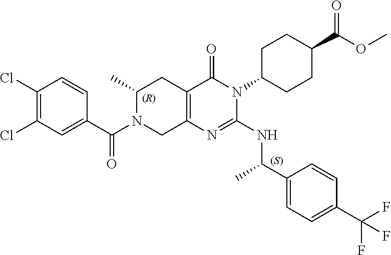 | Cl | 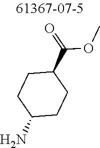 | 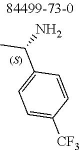 |
| I168 | 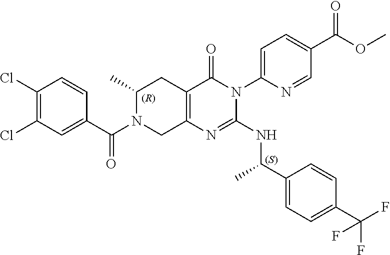 | Cl | 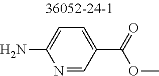 | 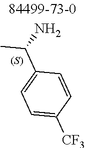 |
| I169 | 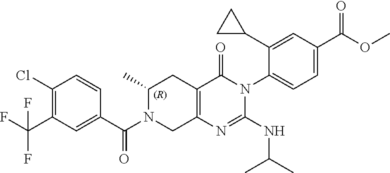 | CF₃ | 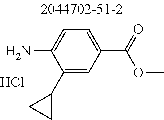 | 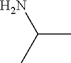 |
| I170 | 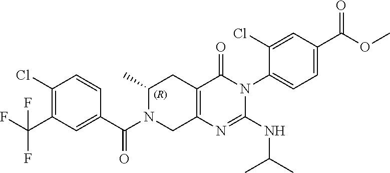 | CF₃ | 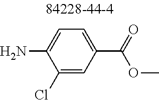 | 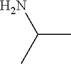 |
| I171 | 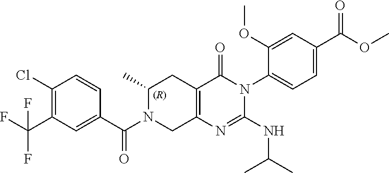 | CF₃ | 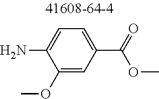 | 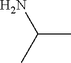 |
| I172 | 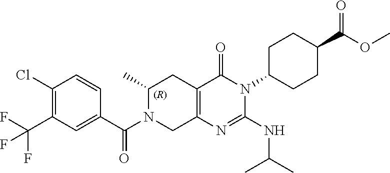 | CF₃ | 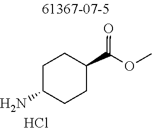 | 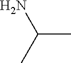 |
| I173 [symbol] | 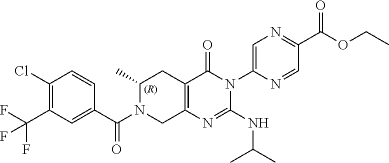 | CF₃ | 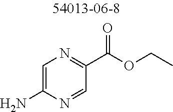 | 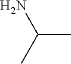 |
| I174 | 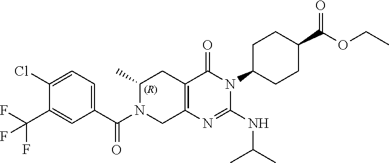 | CF₃ | 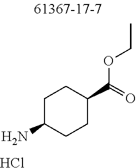 | 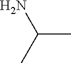 |
| I175 | 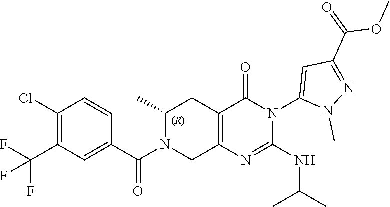 | CF₃ | 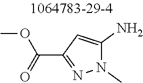 | 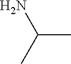 |

^{¥}Only 1 rotamer was detected.
^{£}Few drops of Methylamine were added to the reaction mixture. The rm was stirred and heated at 100° C. for 1 week. Fresh methylamine was added every single 24 hours.
^{≠}Compound C384 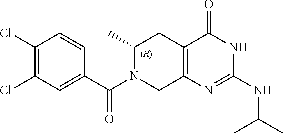
was obtained as by product. [symbol]
Compound C694 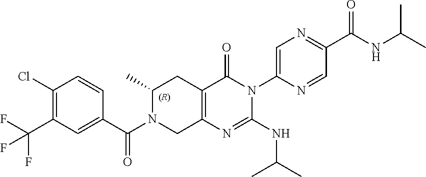
was obtained as a by-product (major) of the reaction (72/28).
^{α}Compound C625 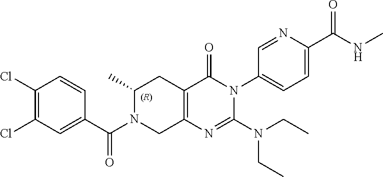
was obtained as a by-product of the reaction (due to contamination of 951247-75-9 by diethylamine).

### SFC Separation

The compounds below have been purified by preparative SFC following one of the methods described in the general information “Separation of rotamers, diastereoisomers and enantiomers”

| Cmpd | |
|---|---|
| no | Structure |
| C383a | 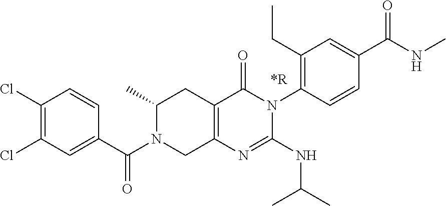 [SMILES: CCc1cc(C(=O)NC)ccc1-n1c(NC(C)C)nc2c(c1=O)C[C@@H](C)N(C(=O)c1ccc(Cl)c(Cl)c1)C2] |
| C383b | 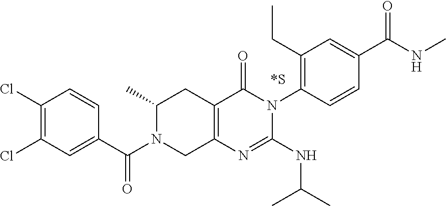 [SMILES: CCc1cc(C(=O)NC)ccc1-n1c(NC(C)C)nc2c(c1=O)C[C@@H](C)N(C(=O)c1ccc(Cl)c(Cl)c1)C2] |
| C386a | 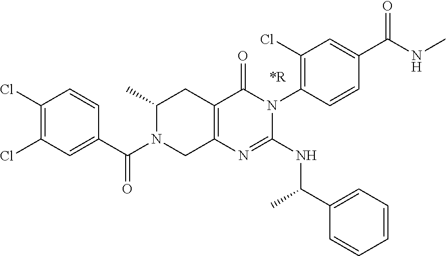 [SMILES: CNC(=O)c1ccc(-n2c(N[C@@H](C)c3ccccc3)nc3c(c2=O)C[C@@H](C)N(C(=O)c2ccc(Cl)c(Cl)c2)C3)c(Cl)c1] |
| C386b | 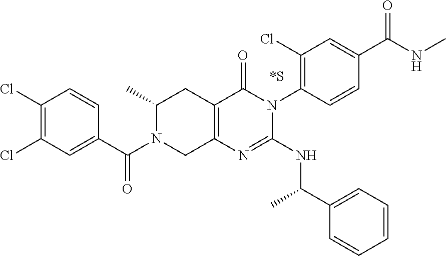 [SMILES: CNC(=O)c1ccc(-n2c(N[C@@H](C)c3ccccc3)nc3c(c2=O)C[C@@H](C)N(C(=O)c2ccc(Cl)c(Cl)c2)C3)c(Cl)c1] |
| C387a | 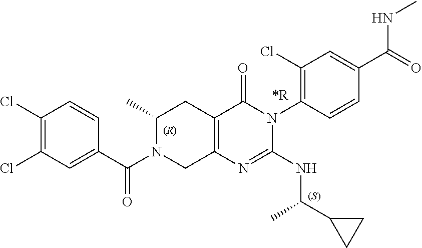 |
| C387b | 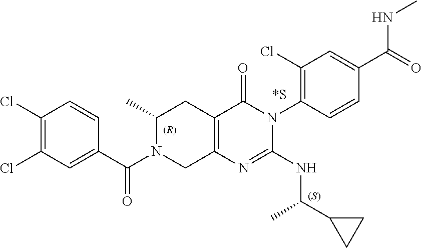 |
| C392a | 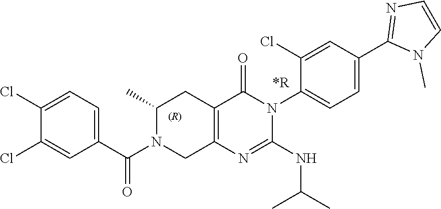 |
| C392b | 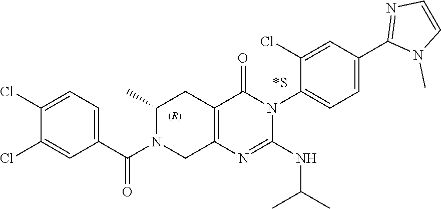 |
| C393a | 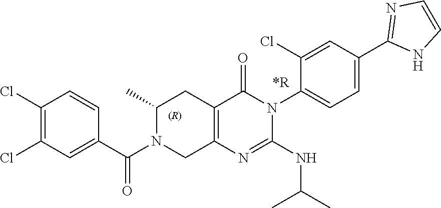 |
| C393b | 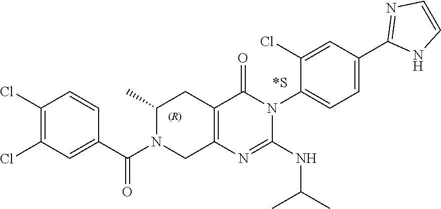 |
| C399a | 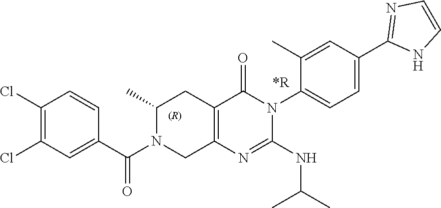 |
| C399b | 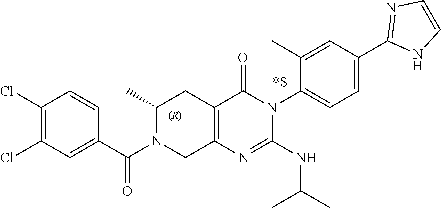 |
| C420a | 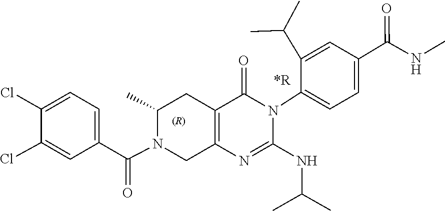 |
| C420b | 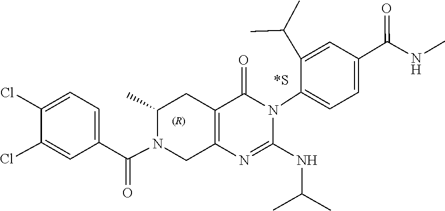 |
| C447a | 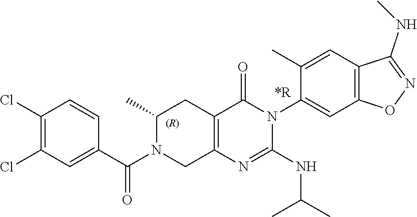 |
| C447b | 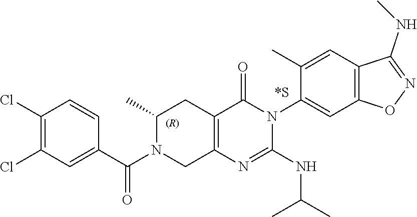 |
| C455a | 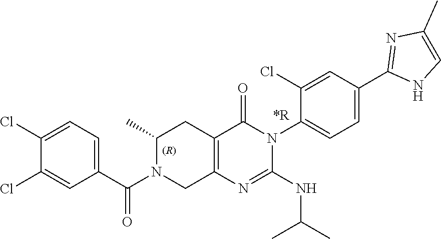 |
| C455b | 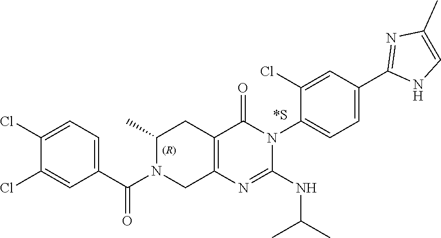 |
| C410b | 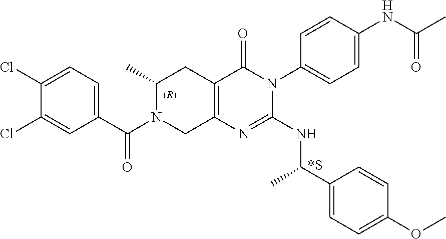 |
| C410a | 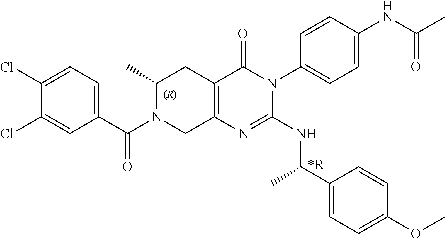 |
| C462a | 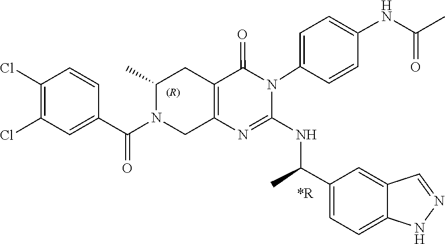 |
| C462b | 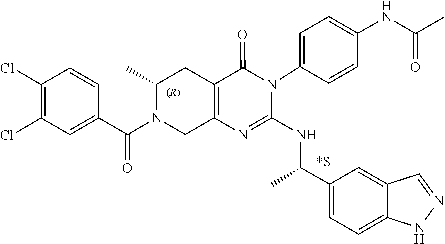 |
| C463a | 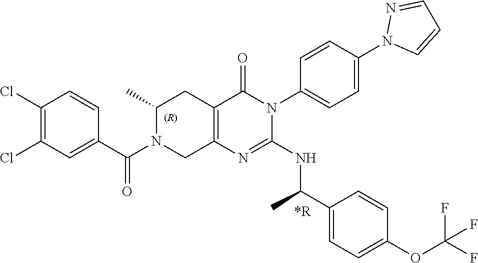 |
| C528a | 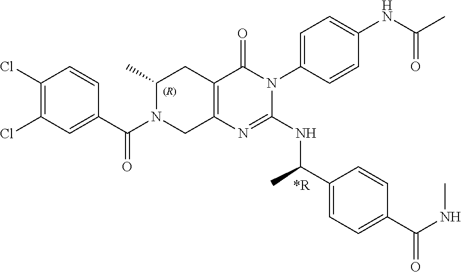 |
| C528b | 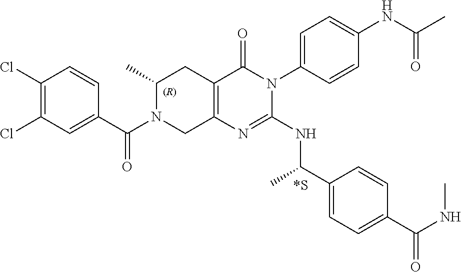 |
| C463b | 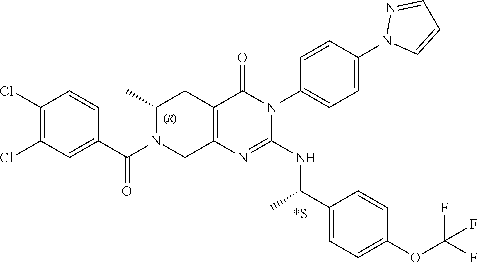 |
| C639a | 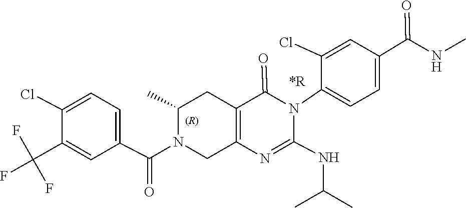 |
| C639b | 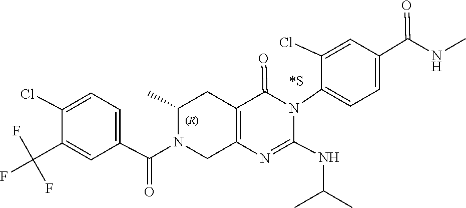 |
| C643a | 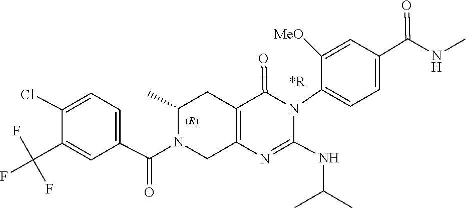 |
| C643b | 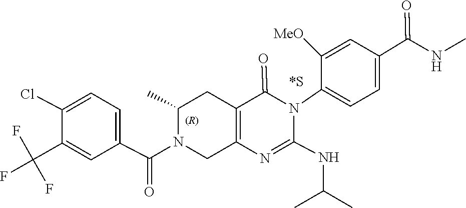 |
| C661a | 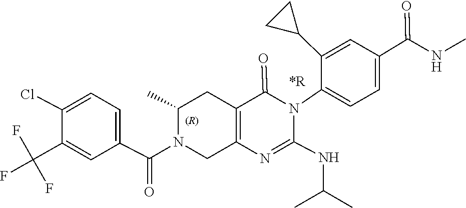 |
| C661b | 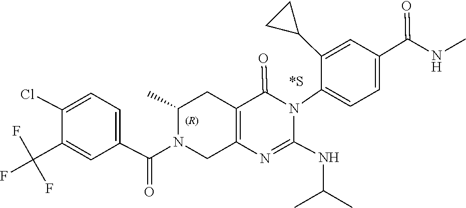 |
| C665a | 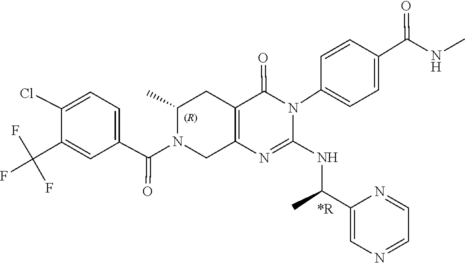 |
| C665b | 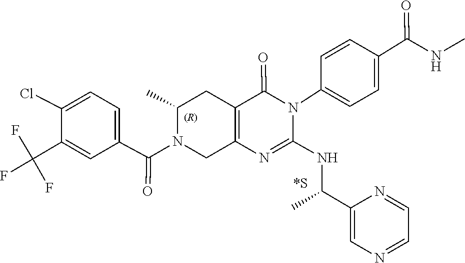 |
| C669a | 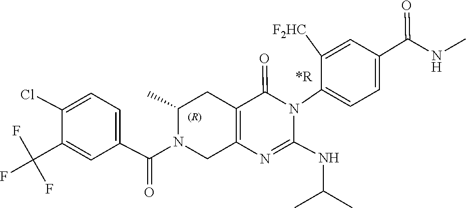 |
| C669b | 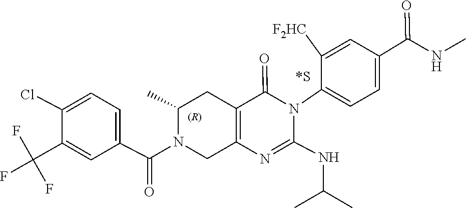 |
| C676a | 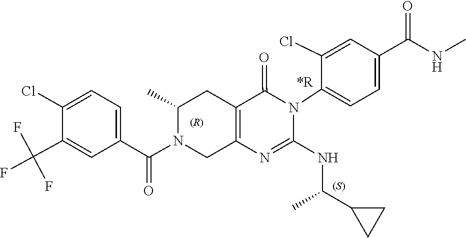 |
| C676b | 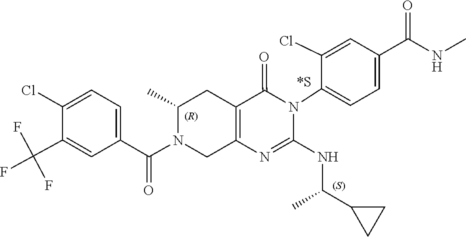 |
| C677b | 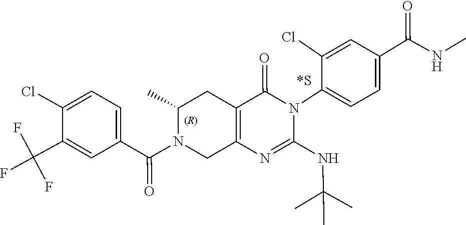 |
| C673a | 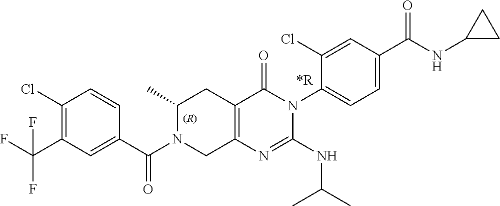 |
| C673b | 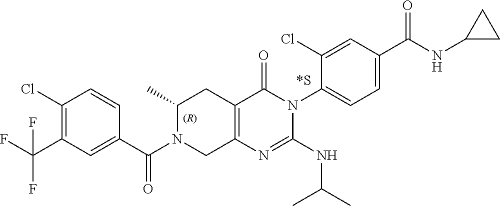 |
| C685a | 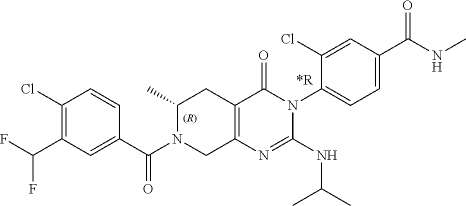 |
| C685b | 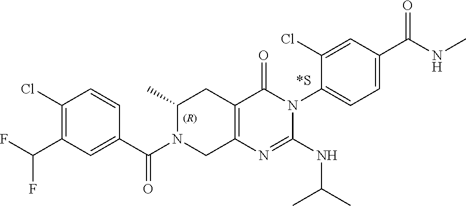 |
| C678a | 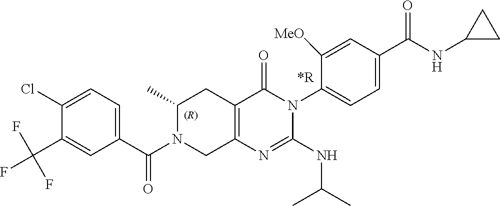 |
| C678b | 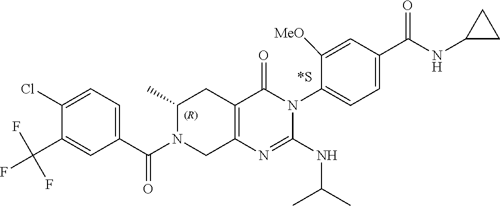 |
| C677a | 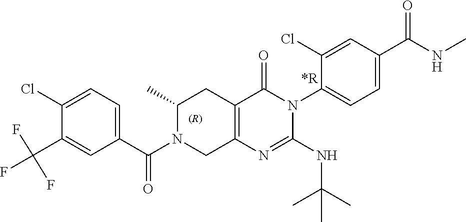 |
| C701a | 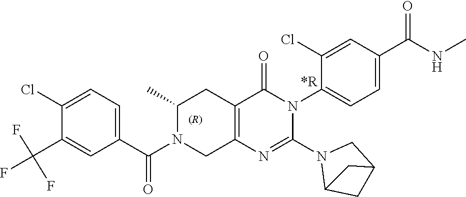 |
| C701b | 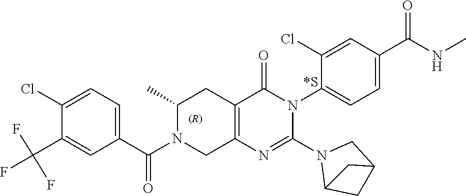 |

### Procedure AU

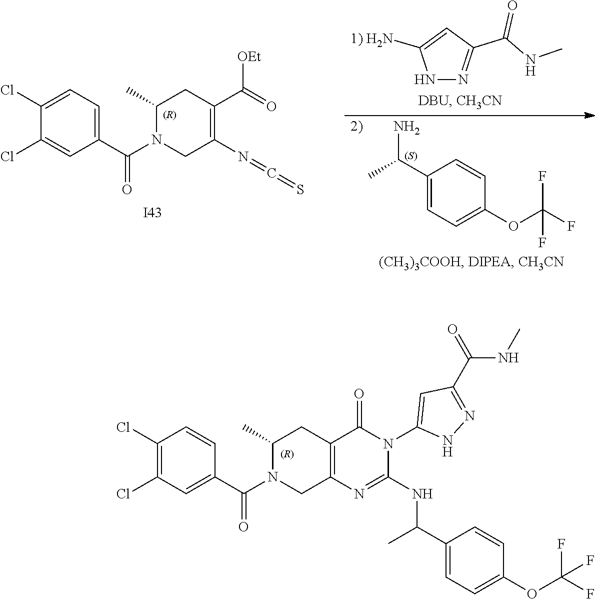

Step 1: I43 (1 g, 2.5 mmol) was dissolved in ACN (9 mL) and 5-amino-N-methyl-1H-pyrazole-3-carboxamide (456 mg, 3.26 mmol) and DBU (0.56 mL, 3.76 mmol) were added. The RM was heated at 80° C. for 5 h. LCMS indicated full conversion. The solvent was removed under reduced pressure, the residue was purified on column chromatography (DCM/MeOH from 100/0 to 90/10) to afford (R)-5-(7-(3,4-dichloro-benzoyl)-6-methyl-4-oxo-2-thioxo-1,4,5,6,7,8-hexahydropyrido[3,4-d]pyrimidin-3(2H)-yl)-N-methyl-1H-pyrazole-3-carboxamide (812 mg, 63% yield which was used as such in next step.

Step 2: To a solution of (R)-5-(7-(3,4-dichlorobenzoyl)-6-methyl-4-oxo-2-thioxo-1,4,5,6,7,8-hexahydropyrido[3,4-d]pyrimidin-3(2H)-yl)-N-methyl-1H-pyrazole-3-carboxamide ((500 mg, 0.973 mmol) in dry ACN (7.6 mL, 145.9 mmol) was added (s)-1-(4-(trifluoromethoxy)phenyl)ethanamine (0.4 mL, 1.95 mmol), dry DIPEA (1 mL, 5.8 mmol) and tert-butyl hydroperoxide in decane (0.53 mL, 5.5 M, 2.9 mmol). The reaction was stirred at RT for 16 h under inert atmosphere. After cooling down, dilution in ACN and filtration, the filtrate was purified via Prep HPLC (Stationary phase: RP XBridge Prep C18 OBD-10 μm, 50×150 mm, Mobile phase: 0.25% NH₄HCO₃ solution in water, CH₃CN). The resulting product was recrystallized in ACN to afford after filtration C607 (130 mg, yield 20%) as a white solid.

The compound listed in the table below were obtained in an analogous manner.

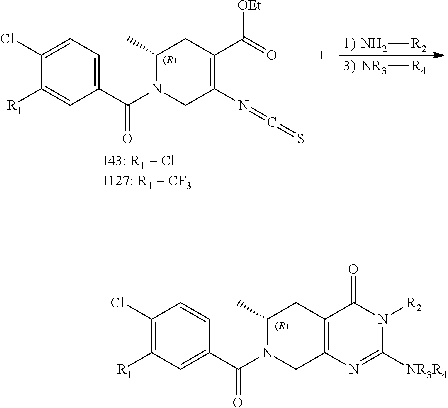

| Cmpd | | | Amine step 1 | Amine Step 3 |
|---|---|---|---|---|
| no | Structure | R | NH₂—R₂ | NR₃R₄ |
| C388 | 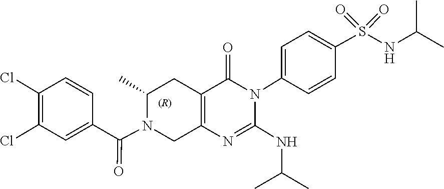 | Cl | 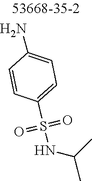 | 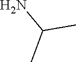 |
| C400 | 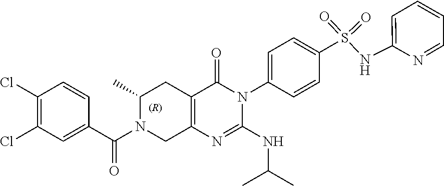 | Cl | 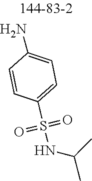 | 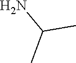 |
| C432 | 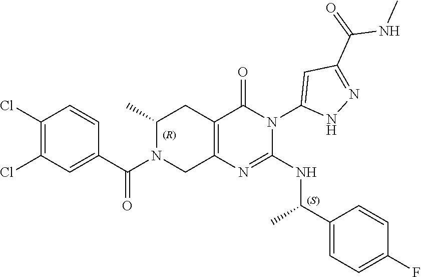 | | 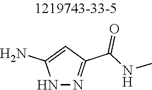 | 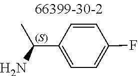 |
| C531 | 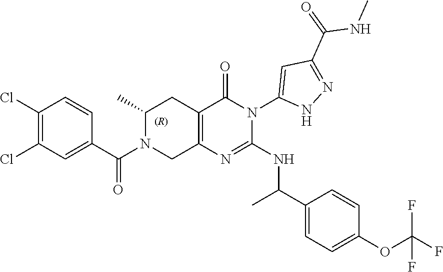 | Cl | 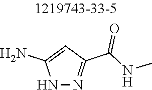 | 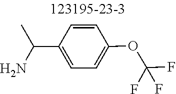 |
| C607 | 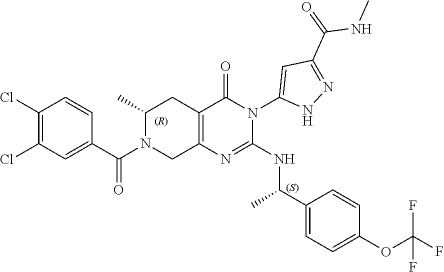 | Cl | 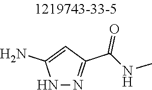 | 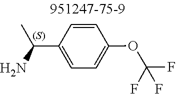 |
| C622 | 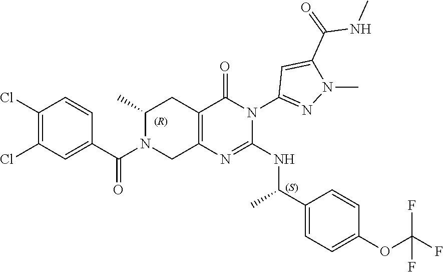 | Cl | 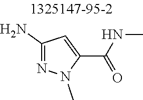 | 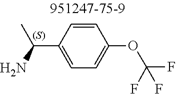 |
| C623 | 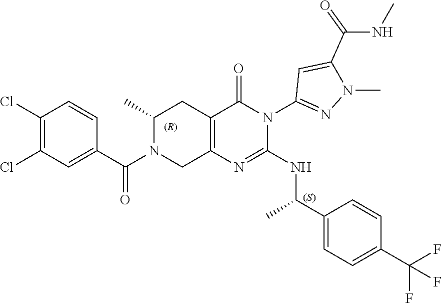 | Cl | 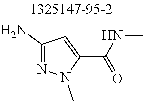 | 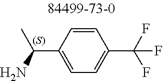 |
| C624 | 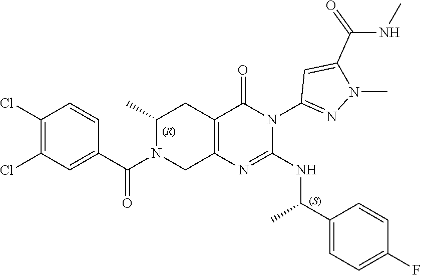 | CF₃ | 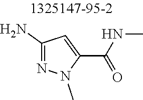 | 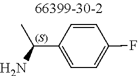 |
| C630 | 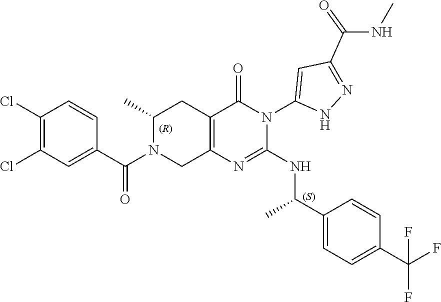 | Cl | 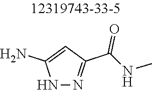 | 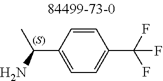 |
| C658^{±} | 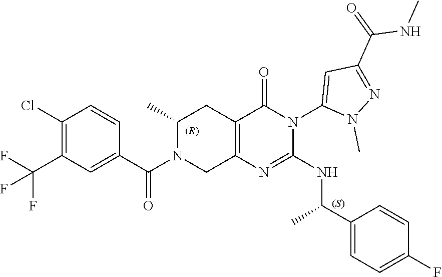 | CF₃ | 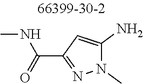 | 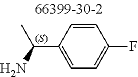 |
| C668 | 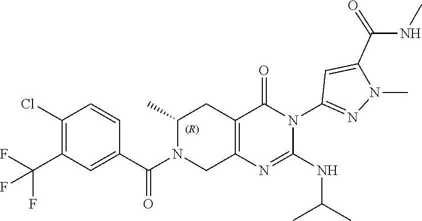 | CF₃ | 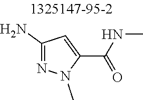 | 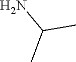 |
| C687 | 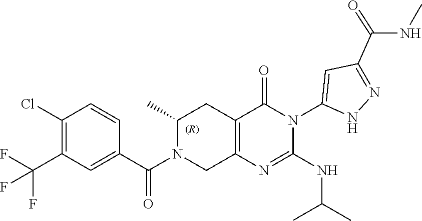 | CF₃ | 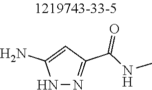 | 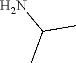 |
| I176 | 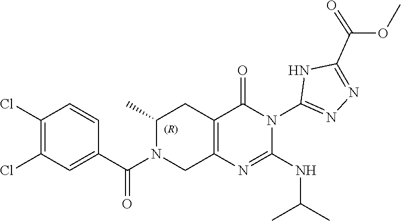 | Cl | 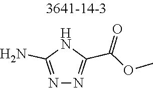 | 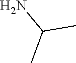 |
| I177 | 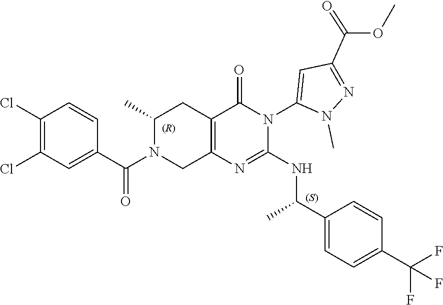 | Cl | 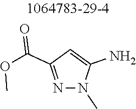 | 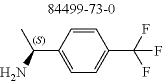 |
| I178 | 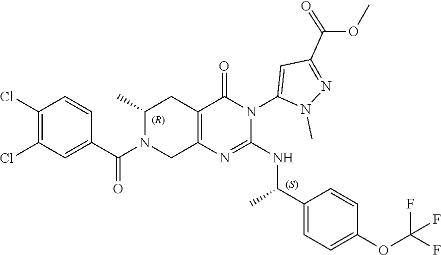 | Cl | 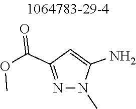 | 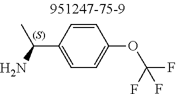 |
| I179 | 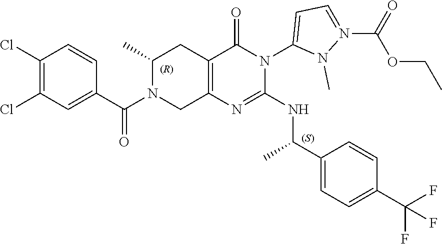 | Cl | 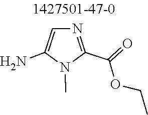 | 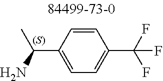 |
| I180 | 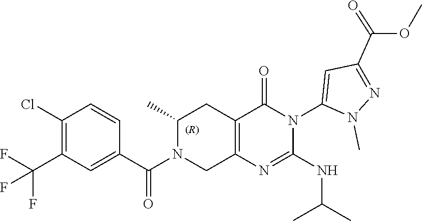 | CF₃ | 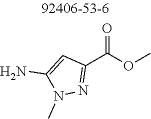 | 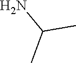 |
| I181 | 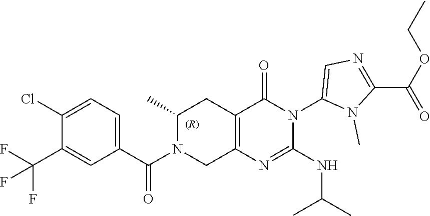 | CF₃ | 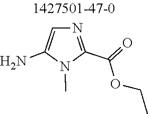 | 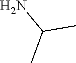 |
| I182 [symbol] | 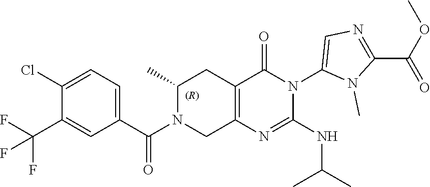 | CF₃ | 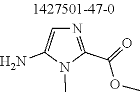 | 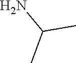 |
| I183 | 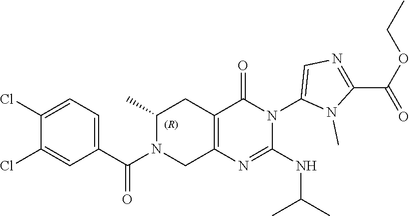 | Cl | 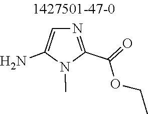 | 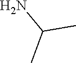 |
| I184 [symbol] | 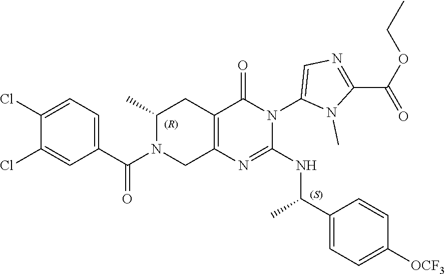 | Cl | 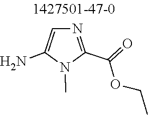 | 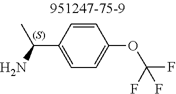 |
| I186 | 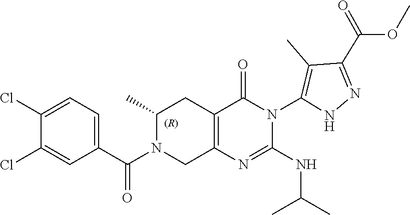 | Cl | 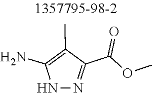 | 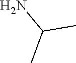 |
| I187 | 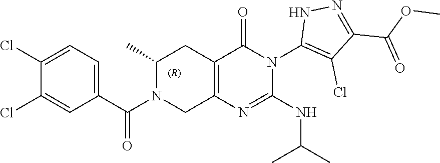 | Cl | 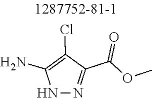 | 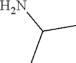 |

^{±}compound C653 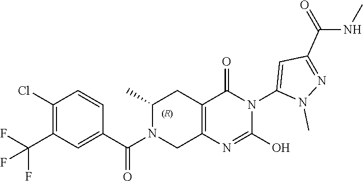
was obtained as a by-product. [symbol]
compound C703 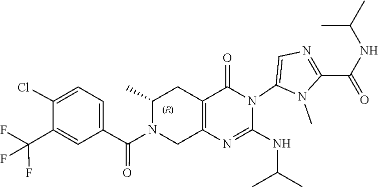
was obtained as a by-product. [symbol]
Intermediate I185 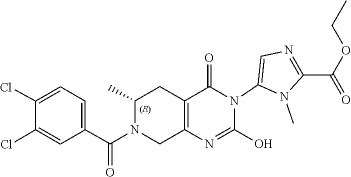
was obtained as a by-product.

### Procedure AV

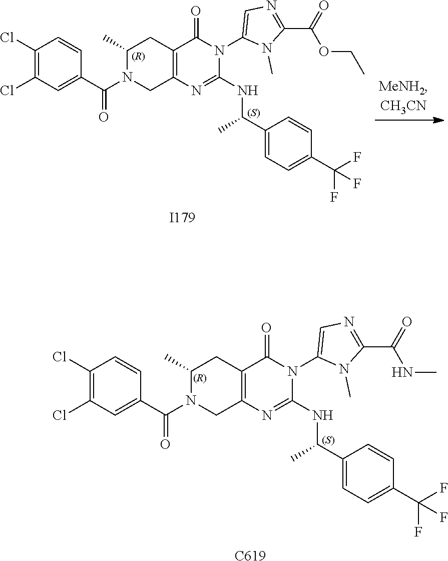

To the solution of I179 (258 mg, 0.38 mmol) in ACN (2 mL), methylamine (118 mg, 3.80 mmol) was added and the reaction mixture was stirred at 80° C. for 16 h. The solvent was removed under reduced pressure. The crude was then purified by column chromatography (DCM/MeOH 100/0 to 95/5) to afford C619 (132 mg, yield 52%).

The compounds listed in the table were obtained in an analogous manner starting from corresponding methyl or ethyl ester.

| Cmpd n° | Structure |
|---|---|
| C374 | 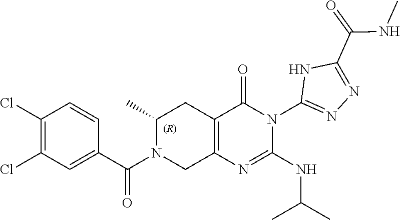 |
| C419 | 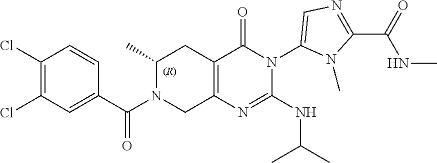 |
| C424 | 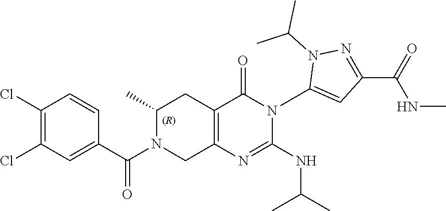 |
| C534 | 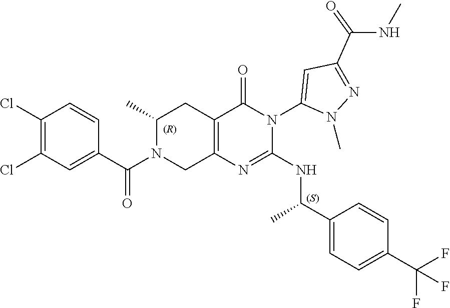 |
| C618 | 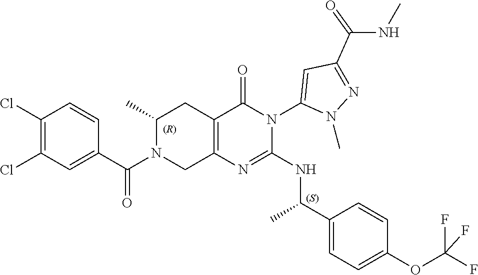 |
| C619 | 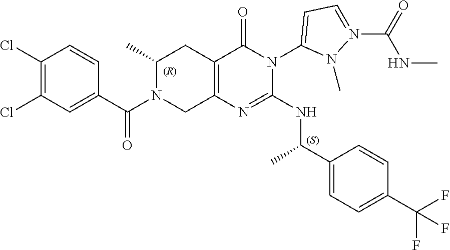 |
| C620 | 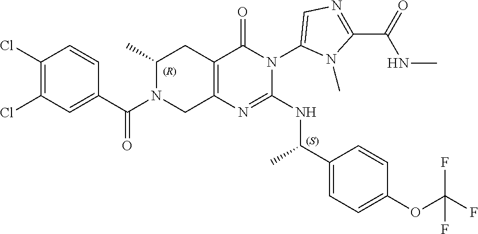 |
| C645^{ψ} | 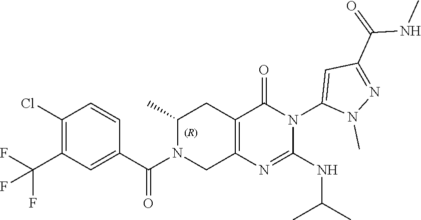 |
| C655 | 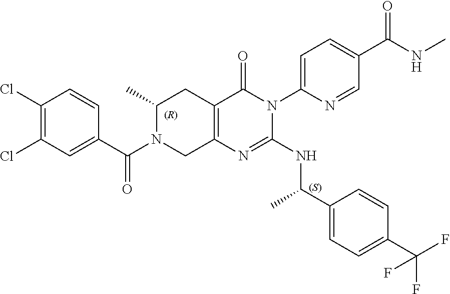 |
| C671^{Δ} | 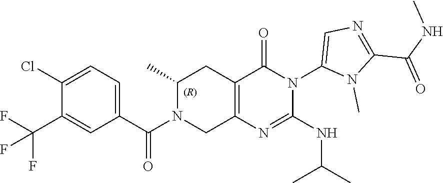 |
| C672 | 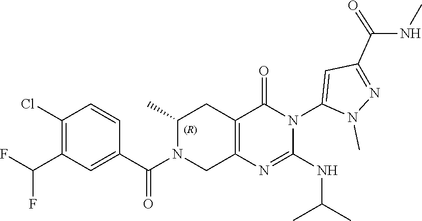 |
| C692 | 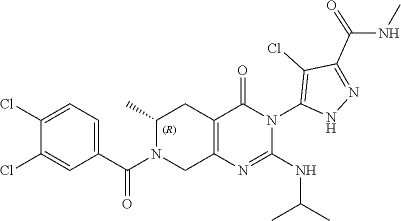 |
| C693 | 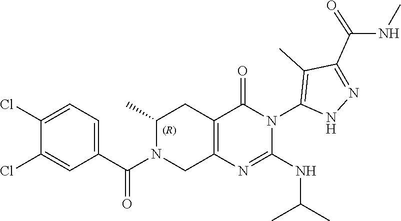 |
| C695 | 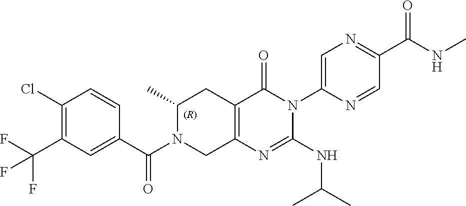 |

^{ψ}compound C646 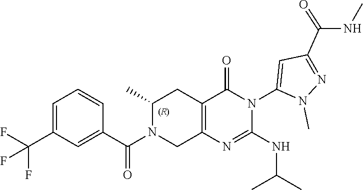
was obtained as a by-product
^{Δ}compound C702 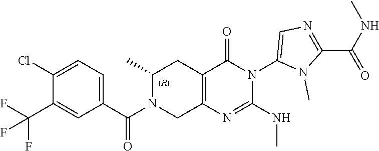
was obtained as a by-product.

### Procedure AW

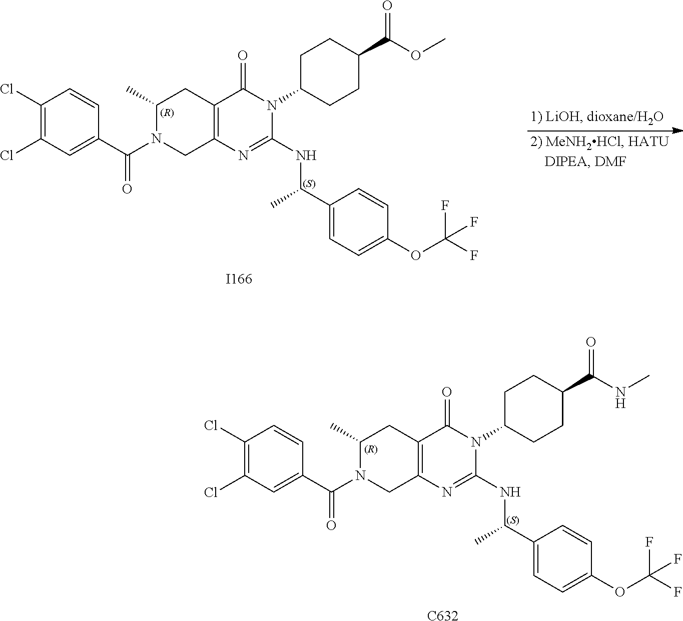

I166 (575 mg, 0.85 mmol) was dissolved in water (1.6 mL) and 1,4-dioxane (4.8 mL). Lithium hydroxide monohydrate (70.8 mg, 1.69 mmol) was added and the mixture was stirred at RT for 1 h. The mixture was concentrated and the residue dissolved in water. HCl (1.69 mL, 1 M in H₂O, 1.69 mmol) was added and the mixture was extracted with Me-THF. The organic layer was dried over Na₂SO₄), filtered and concentrated in vacuo to afford (1S,4r)-4-((R)-7-(3,4-dichlorobenzoyl)-6-methyl-4-oxo-2-(((S)-1-(4-(trifluoromethoxy)phenyl)ethyl)amino)-5,6,7,8-tetrahydropyrido[3,4-d]pyrimidin-3(4H)-yl)cyclohexane-1-carboxylic acid (517 mg, yield 92%).

A tube was charged with (1S,4r)-4-((R)-7-(3,4-dichlorobenzoyl)-6-methyl-4-oxo-2-(((S)-1-(4-(trifluoromethoxy)phenyl)ethyl)amino)-5,6,7,8-tetrahydropyrido[3,4-d]pyrimidin-3(4H)-yl)cyclohexane-1-carboxylic acid (517 mg, 0.78 mmol) in DMF (5 mL). DIPEA (0.17 mL, 1.01 mmol) and HATU (353 mg, 0.93 mmol) was added and the mixture was stirred at RT for 1 h. Methylamine hydrochloride (28.87 mg, 0.93 mmol) was added and the mixture was stirred at RT for 1 h. The mixture was concentrated in vacuo. The residue was purified on silica column chromatography (heptane/EtOAc from 100/0 to 0/100). The obtained product was crystallized from water-MeOH, filtered off and dried under vacuum to afford C632 (378 mg, yield 72%) as a white solid.

The compounds listed in the table were obtained in an analogous manner starting from corresponding methyl or ethyl ester.

| Cmpd n° | Structure |
|---|---|
| C456 | 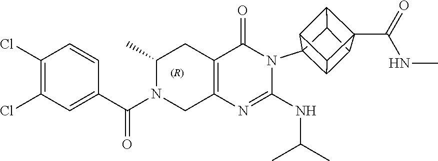 |
| C632 | 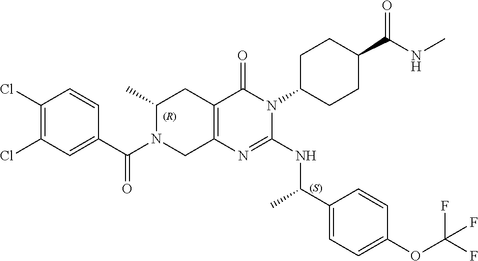 |
| C640 | 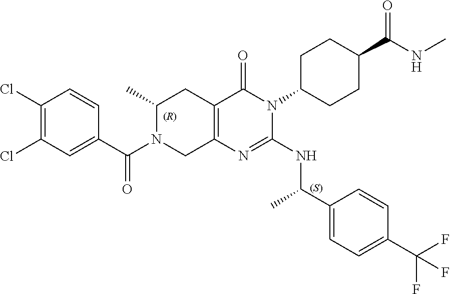 |
| C661 | 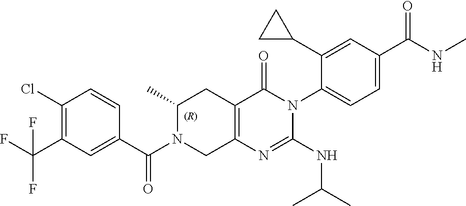 |
| C673 | 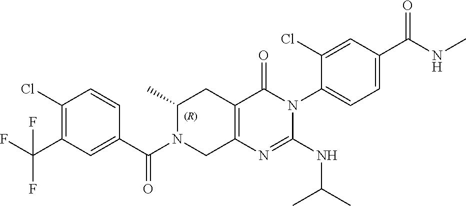 |
| C678 | 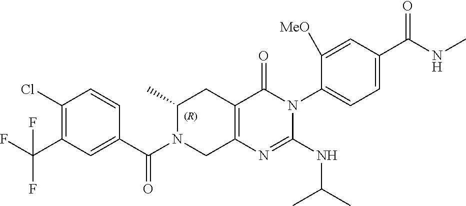 |
| C690 | 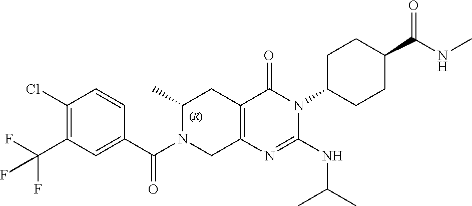 |
| C696 | 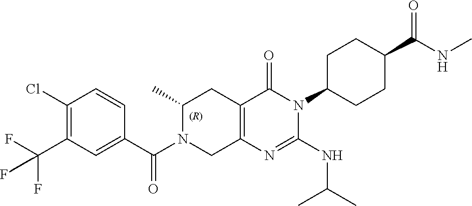 |
| C700 | 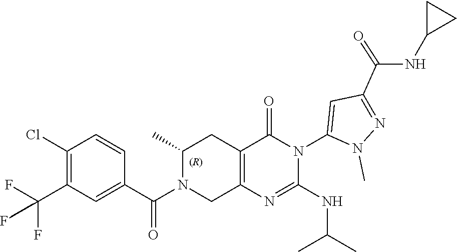 |

### Procedure AX

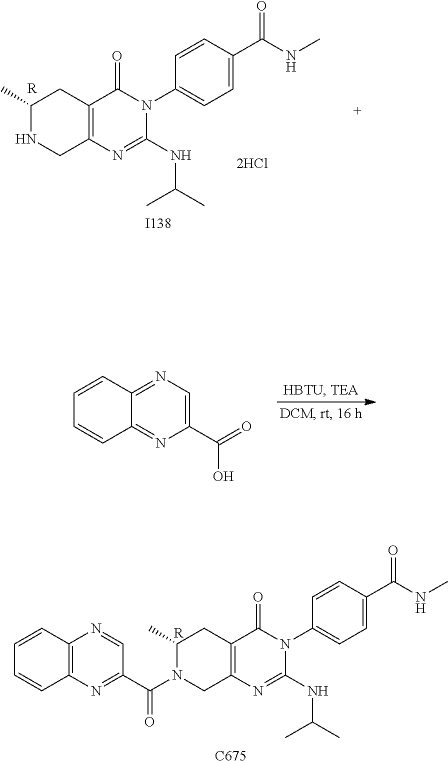

(R)-4-(2-(isopropylamino)-6-methyl-4-oxo-5,6,7,8-tetrahydropyrido[3,4-d]pyrimidin-3(4H)-yl)-N-methylbenzamid.2HCl I138 (201 mg, 0.47 mmol) was added to a solution of 2-quinoxalinecarboxylic acid (243 mg, 1.40 mmol), dry TEA (0.6 mL, 4.32 mmol) and HBTU (288 mg, 0.76 mmol) in dry DCM (5 mL). The mixture was stirred at room temperature for 16 h. The solvent was removed and the crude was purified via Prep HPLC (Stationary phase: RP XBridge Prep C18 OBD-10 μm, 50×150 mm, Mobile phase: 0.25% NH₄HCO₃ solution in water, CH₃CN). The obtained product was recrystallized in acetone and water. The crystals were collected by filtration to get C675 (155 mg, yield 65%) as a white powder.

In analogous manner, the compounds below were synthetizes starting from intermediate I42 or I138 and the appropriate corresponding carboxylic acid.

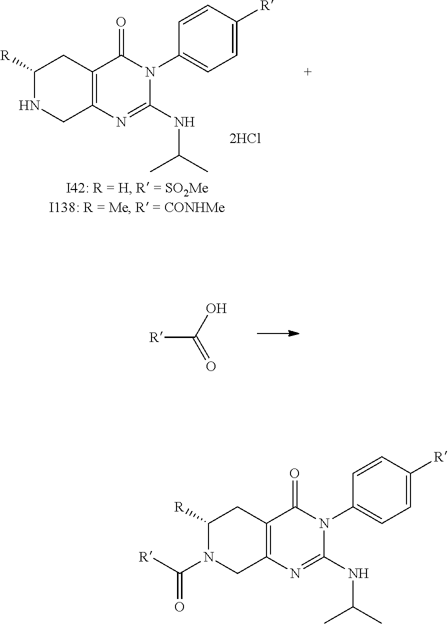

| Cmpd n° | Structure |
|---|---|
| C356 | 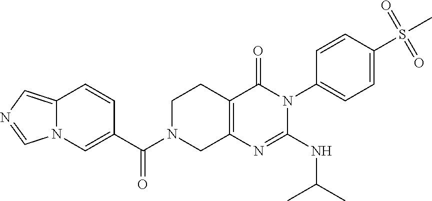 [SMILES: CC(C)Nc1nc2c(c(=O)n1-c1ccc(S(C)(=O)=O)cc1)CCN(C(=O)c1ccc3cncn3c1)C2] |
| C357 | 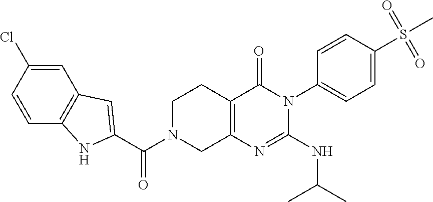 [SMILES: CC(C)Nc1nc2c(c(=O)n1-c1ccc(S(C)(=O)=O)cc1)CCN(C(=O)c1cc3cc(Cl)ccc3[nH]1)C2] |
| C358 | 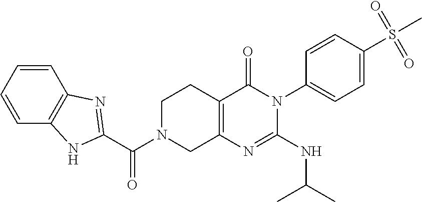 [SMILES: CC(C)Nc1nc2c(c(=O)n1-c1ccc(S(C)(=O)=O)cc1)CCN(C(=O)c1nc3ccccc3[nH]1)C2] |
| C359 | 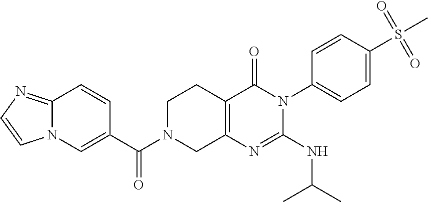 [SMILES: CC(C)Nc1nc2c(c(=O)n1-c1ccc(S(C)(=O)=O)cc1)CCN(C(=O)c1ccc3nccn3c1)C2] |
| C360 | 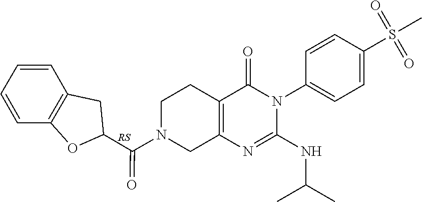 [SMILES: CC(C)Nc1nc2c(c(=O)n1-c1ccc(S(C)(=O)=O)cc1)CCN(C(=O)C1Cc3ccccc3O1)C2] |
| C361 | 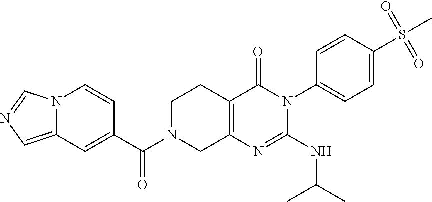 [SMILES: CC(C)Nc1nc2c(c(=O)n1-c1ccc(S(C)(=O)=O)cc1)CCN(C(=O)c1ccn3cncc3c1)C2] |
| C362 | 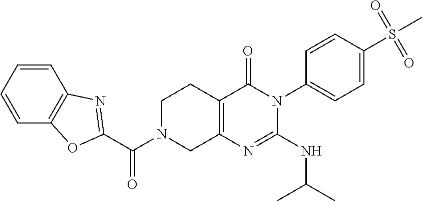 [SMILES: CC(C)Nc1nc2c(c(=O)n1-c1ccc(S(C)(=O)=O)cc1)CCN(C(=O)c1nc3ccccc3o1)C2] |
| C363 | 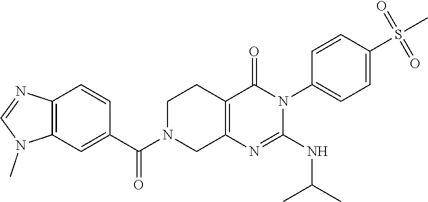 [SMILES: CC(C)Nc1nc2c(c(=O)n1-c1ccc(S(C)(=O)=O)cc1)CCN(C(=O)c1ccc3ncn(C)c3c1)C2] |
| C364 | 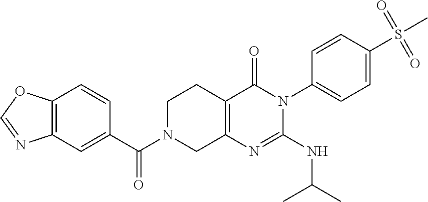 [SMILES: CC(C)Nc1nc2c(c(=O)n1-c1ccc(S(C)(=O)=O)cc1)CCN(C(=O)c1ccc3ocnc3c1)C2] |
| C365 | 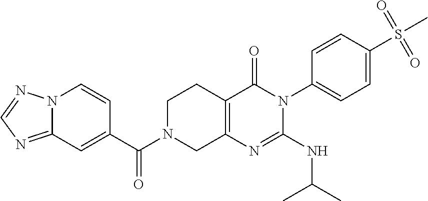 [SMILES: CC(C)Nc1nc2c(c(=O)n1-c1ccc(S(C)(=O)=O)cc1)CCN(C(=O)c1ccn3ncnc3c1)C2] |
| C366 | 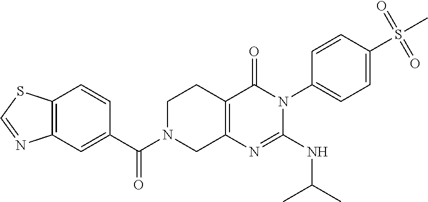 [SMILES: CC(C)Nc1nc2c(c(=O)n1-c1ccc(S(C)(=O)=O)cc1)CCN(C(=O)c1ccc3scnc3c1)C2] |
| C367 | 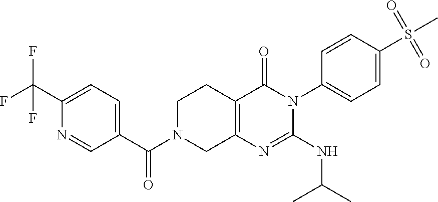 [SMILES: CC(C)Nc1nc2c(c(=O)n1-c1ccc(S(C)(=O)=O)cc1)CCN(C(=O)c1ccc(C(F)(F)F)nc1)C2] |
| C368 | 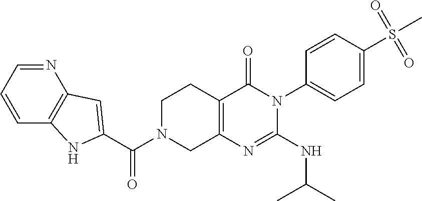 [SMILES: CC(C)Nc1nc2c(c(=O)n1-c1ccc(S(C)(=O)=O)cc1)CCN(C(=O)c1cc3ncccc3[nH]1)C2] |
| C369 | 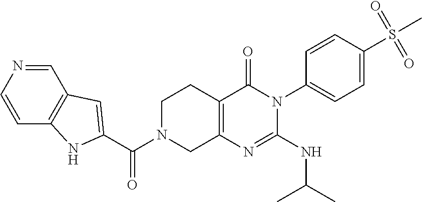 [SMILES: CC(C)Nc1nc2c(c(=O)n1-c1ccc(S(C)(=O)=O)cc1)CCN(C(=O)c1cc3cnccc3[nH]1)C2] |
| C418 | 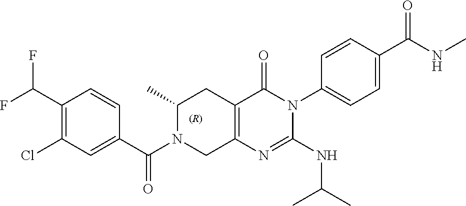 |
| C427 | 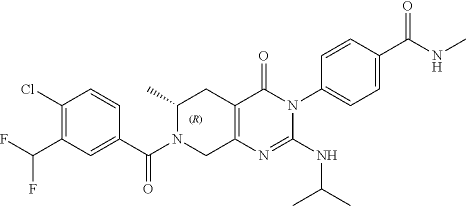 |
| C428 | 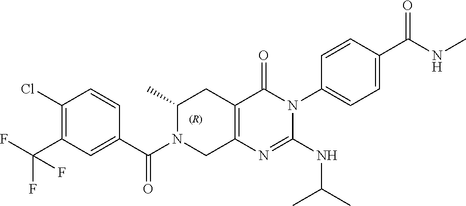 |
| C429 | 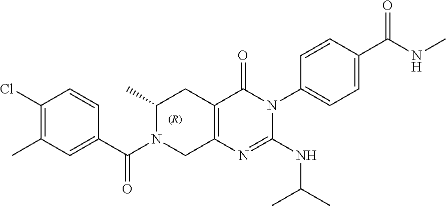 |
| C475 | 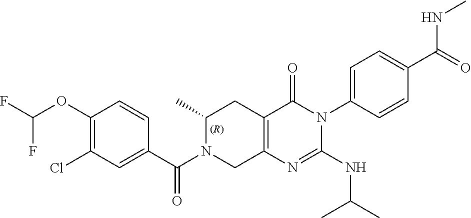 |
| C525 | 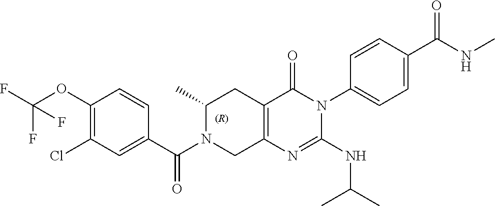 |
| C606 | 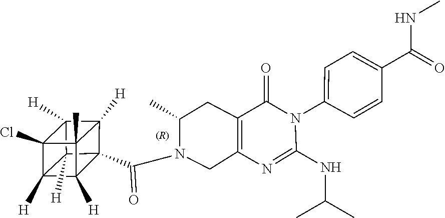 |
| C634 | 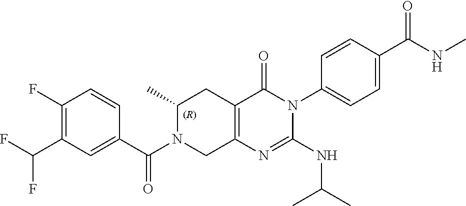 |
| C635 | 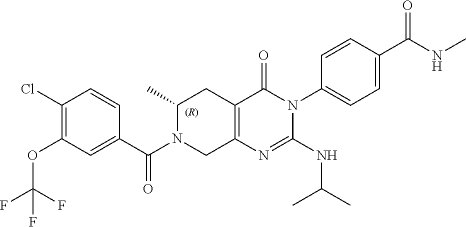 |
| C641 | 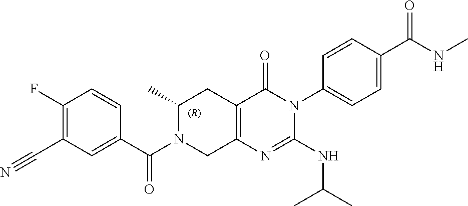 |
| C648 | 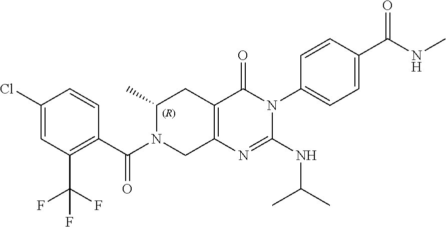 |
| C674 | 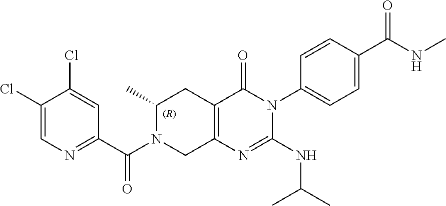 |
| C675 | 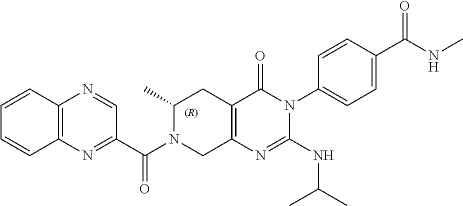 |
| C679 | 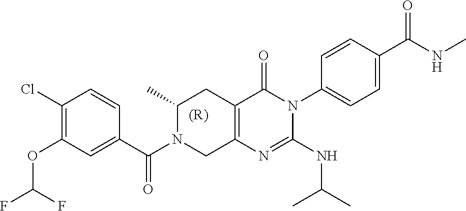 |
| C686 | 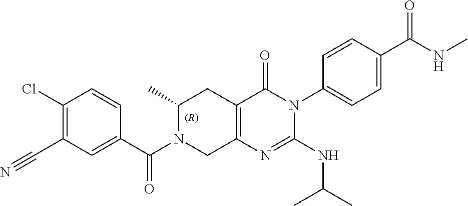 |

### Procedure AY

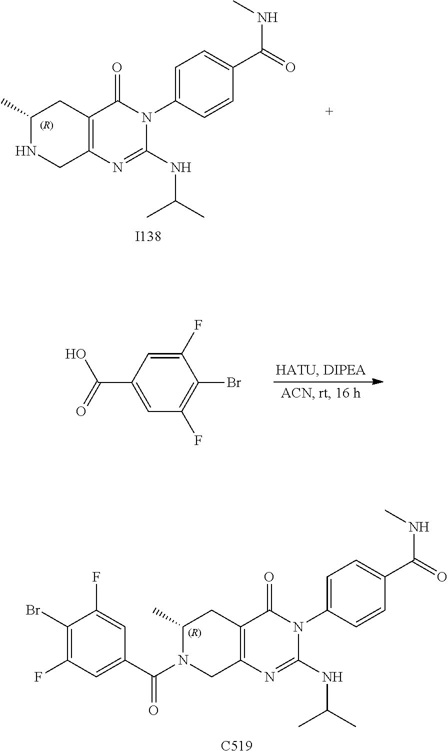

The vial was charged with (R)-4-(2-(isopropylamino)-6-methyl-4-oxo-5,6,7,8-tetrahydropyrido[3,4-d]pyrimidin-3(4H)-yl)-N-methylbenzamide I138 (36 mg, 0.10 mmol), 4-bromo-3,5-difluorobenzoic acid (26 mg, 0.11 mmol) and DIPEA (0.0431 mL, 0.25 mmol, 0.75 g/mL). Then HATU (39.92 mg, 0.11 mmol) was added in one portion. The reaction mixture was left to stand for 16 h at ambient temperature. The solvent was evaporated, the residue was dissolved in DMSO and subjected to HPLC purification♦♦

to obtain the title compound C519 (17 mg, yield 30%).

♦♦The purification was performed using Agilent 1260 Infinity systems equipped with DAD and mass-detector. Waters Sunfire C18 OBD Prep Column, 100 A, 5 μm, 19 mm×100 mm with SunFire C18 Prep Guard Cartridge, 100 A, 10 μm, 19 mm×10 mm was used. Deionized Water (phase A) and HPLC-grade Methanol (phase B) were used as an eluent. In some cases, ammonia or TFA was used as an additive to improve the separation of the products. In these cases, free bases and TFA salts of the products were formed respectively.

The compound listed in the table below were synthetized in an analogous manner.

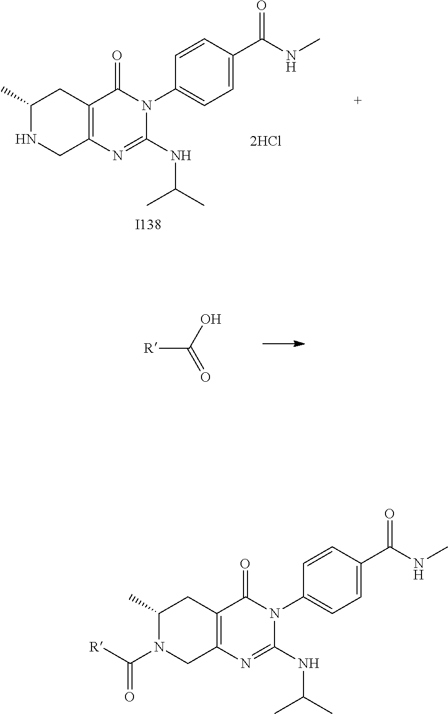

| Cmpd n° | Structure |
|---|---|
| C591 | 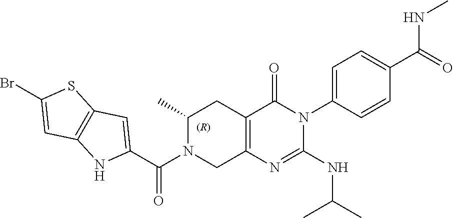 |
| C570 | 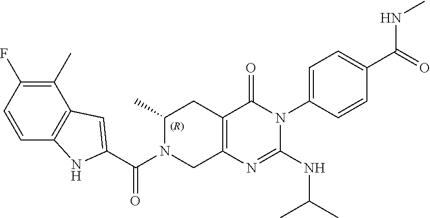 |
| C569 | 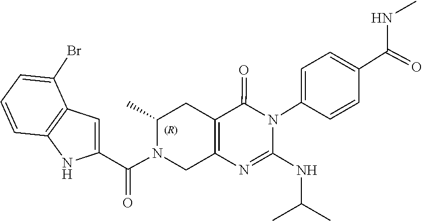 |
| C572 | 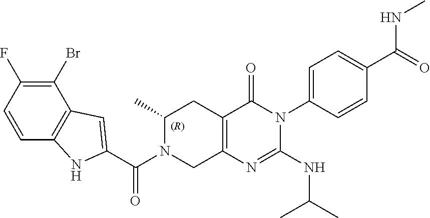 |
| C519 | 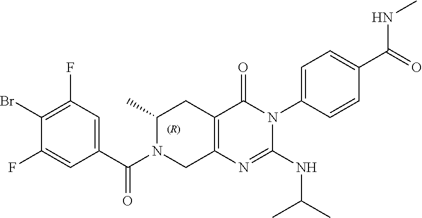 |
| C564 | 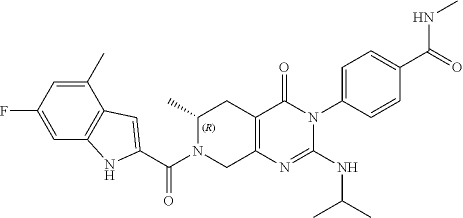 |
| C505 | 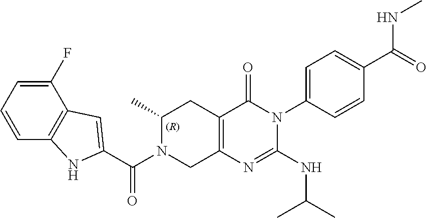 |
| C568 | 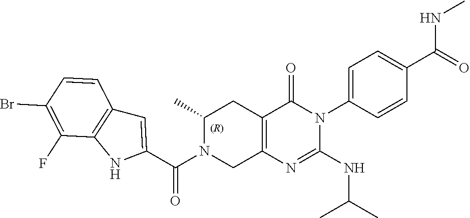 |
| C566 | 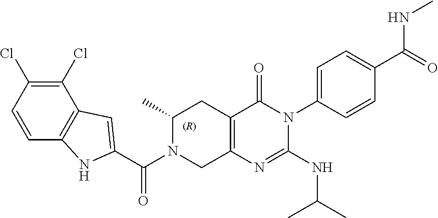 |
| C594 | 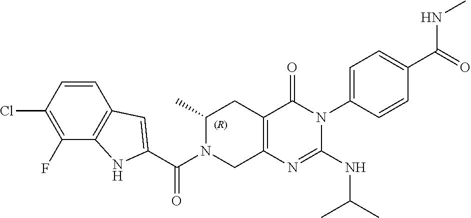 |
| C589 | 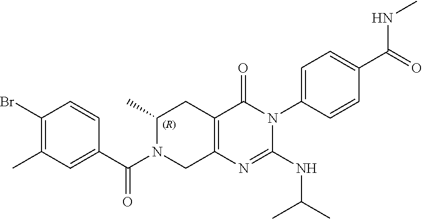 |
| C576 | 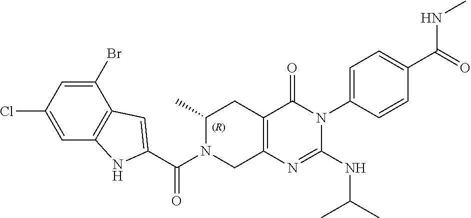 |
| C475 | 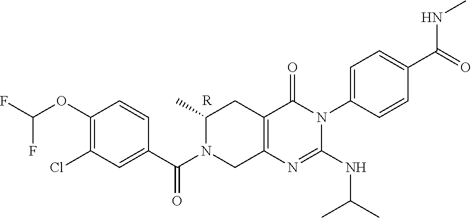 |
| C580 | 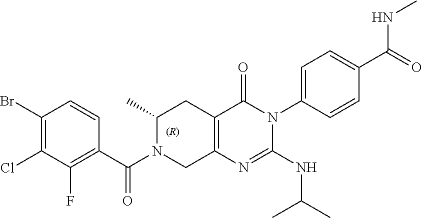 |
| C587 | 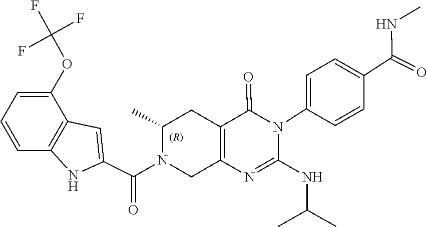 |
| C571 | 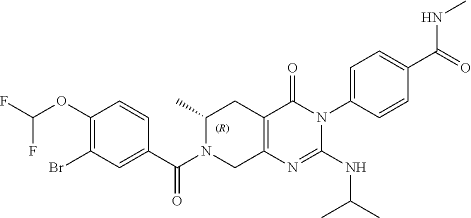 |
| C585 | 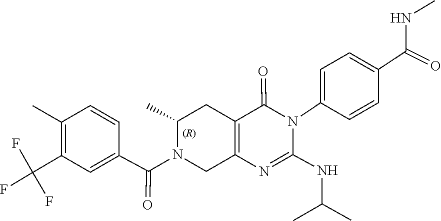 |
| C584 | 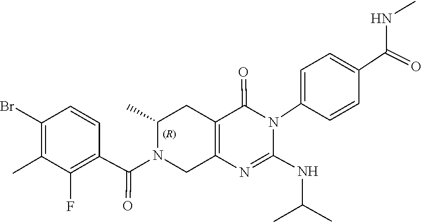 |
| C578 | 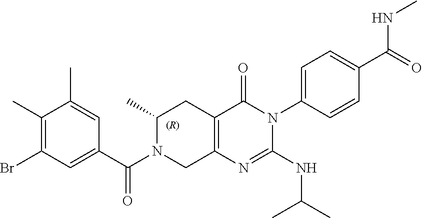 |
| C476 | 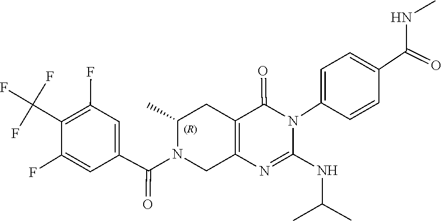 |
| C579 | 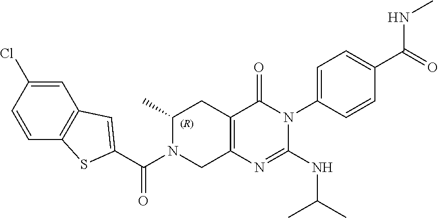 |
| C581 | 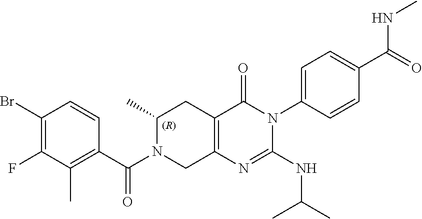 |
| C577 | 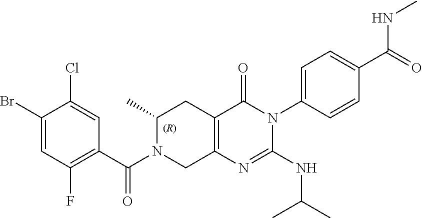 |
| C586 | 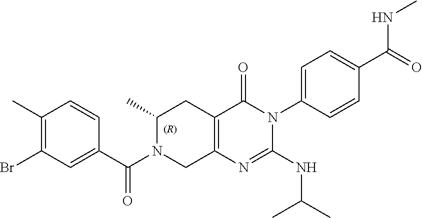 |
| C583 | 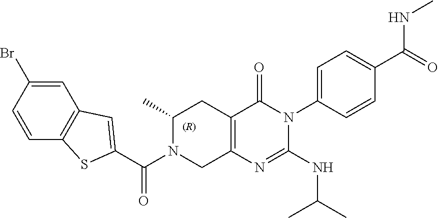 |
| C471 | 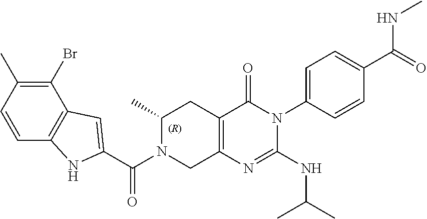 |
| C563 | 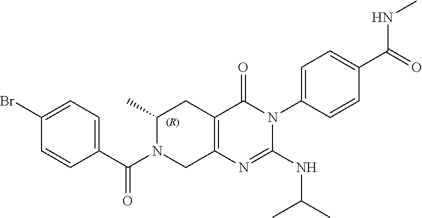 |
| C573 | 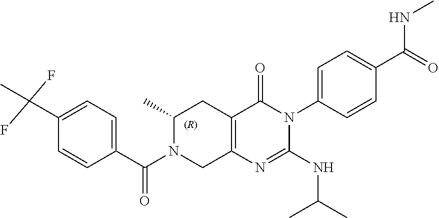 |
| C501 | 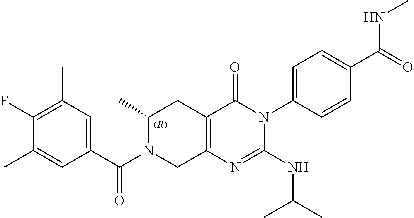 |
| C602 | 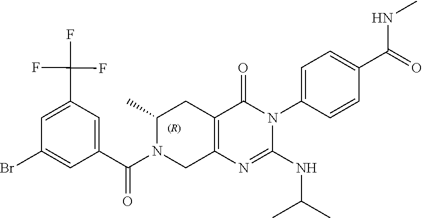 |
| C575 | 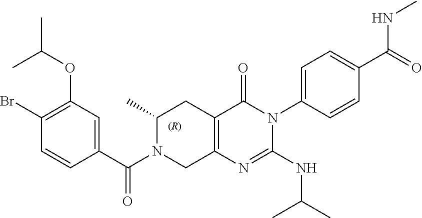 |
| C500 | 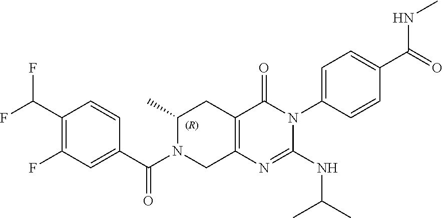 |
| C514 | 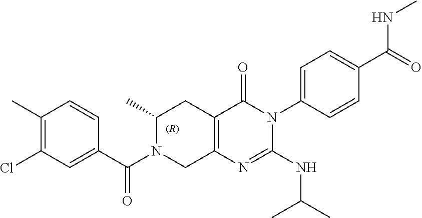 |
| C567 | 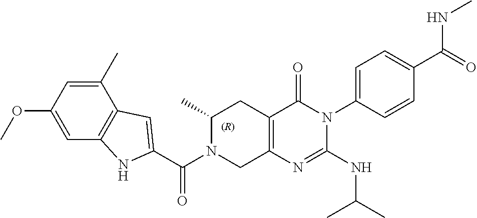 |
| C565 | 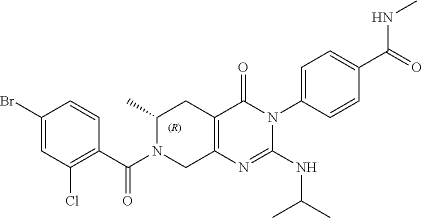 |
| C592 | 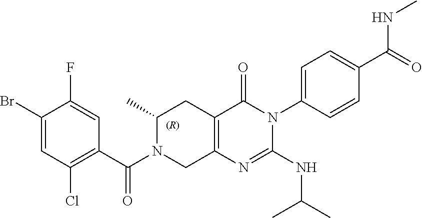 |
| C574 | 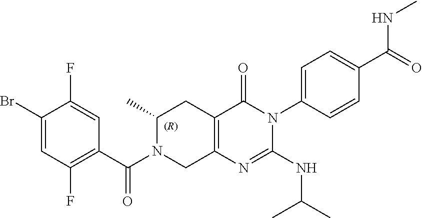 |
| C522 | 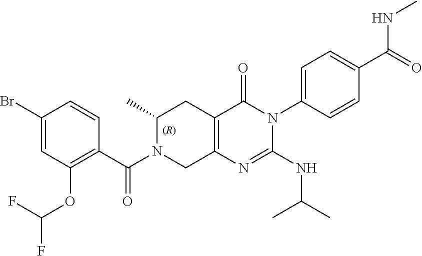 |
| C508 | 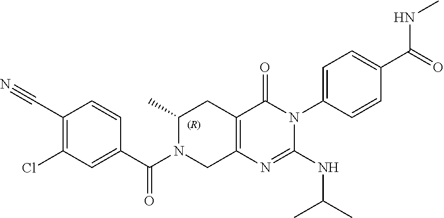 |
| C472 | 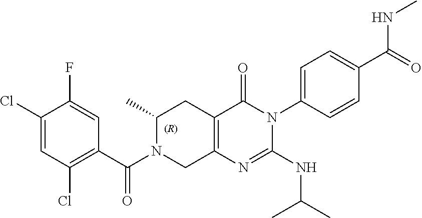 |
| C506 | 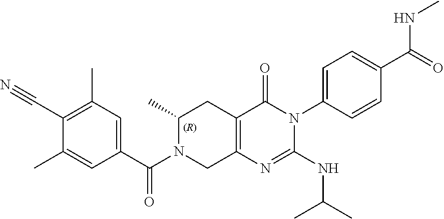 |
| C504 | 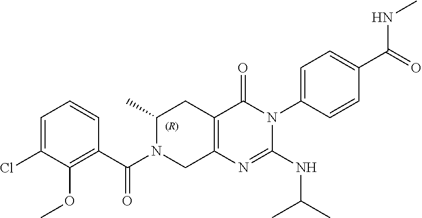 |
| C512 | 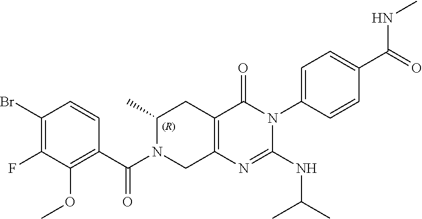 |
| C515 | 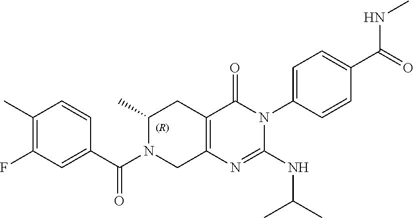 |
| C478 | 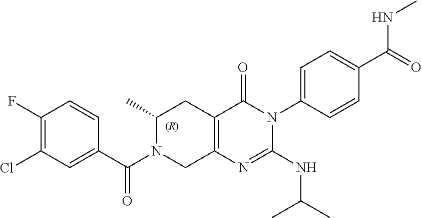 |
| C524 | 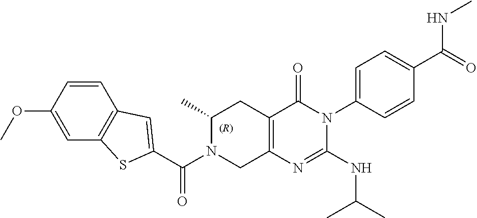 |
| C499 | 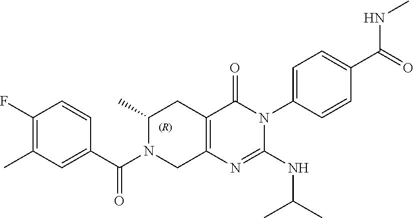 |
| C588 | 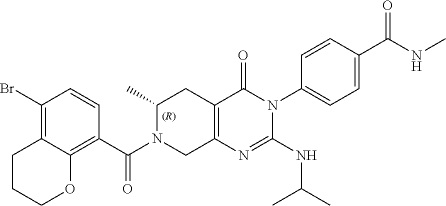 |
| C492 | 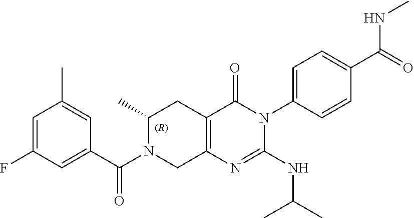 |
| C582 | 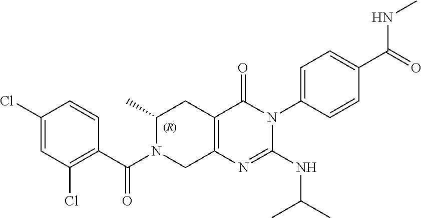 |
| C511 | 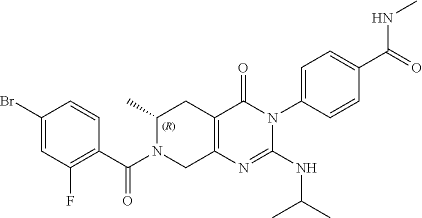 |
| C494 | 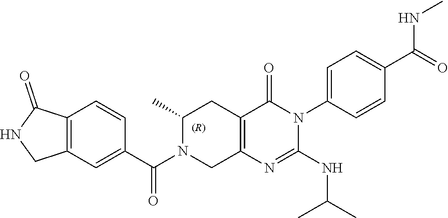 |
| C516 | 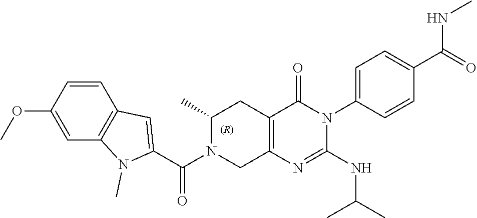 |
| C507 | 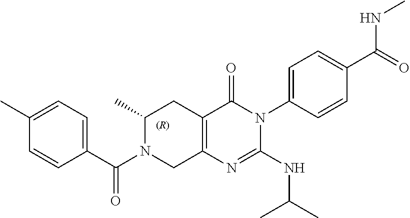 |
| C498 | 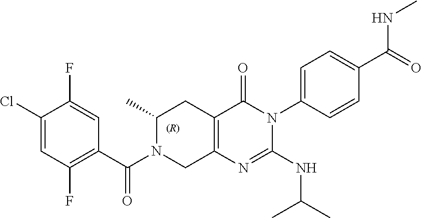 |
| C520 | 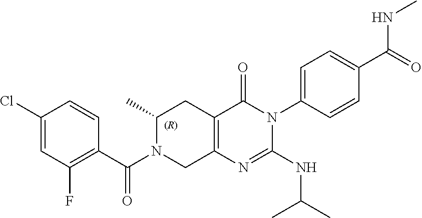 |
| C503 | 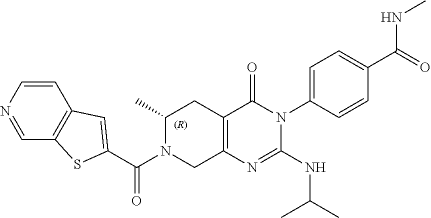 |
| C521 | 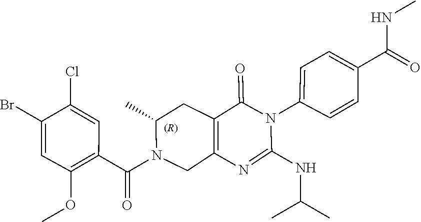 |
| C502 | 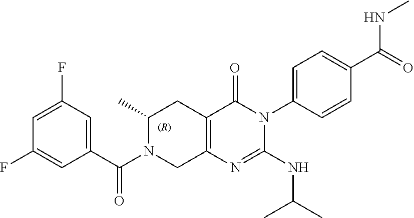 |
| C510 | 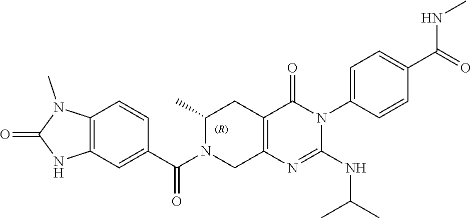 |
| C477 | 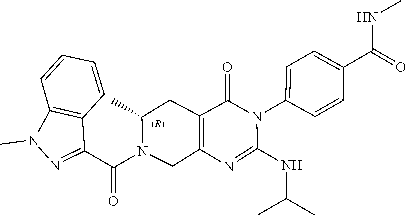 |
| C497 | 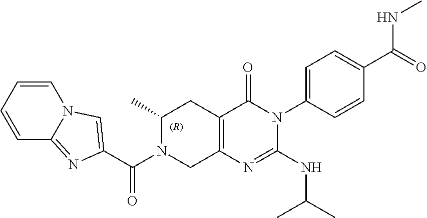 |
| C590 | 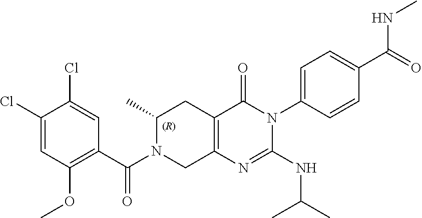 |
| C493 | 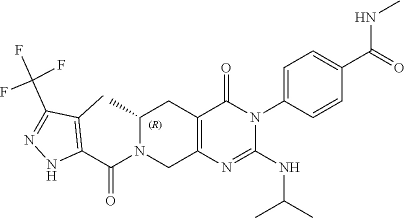 |
| C491 | 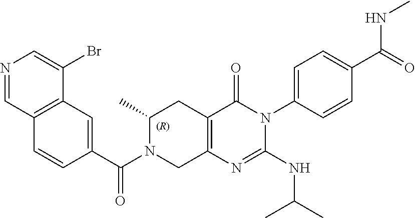 |
| C523 | 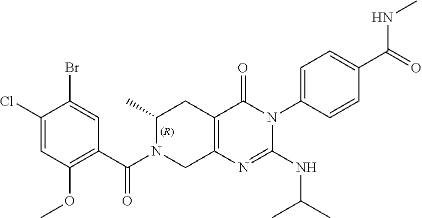 |
| C513 | 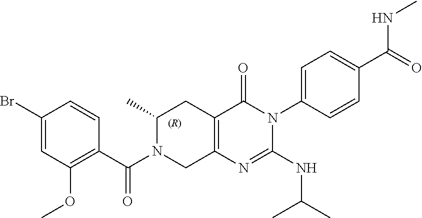 |
| C593 | 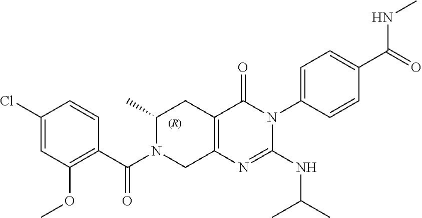 |
| C604 | 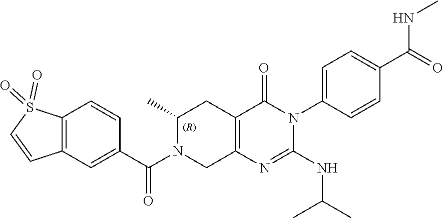 |
| C473 | 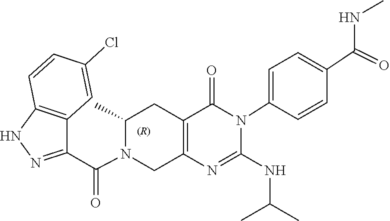 |
| C474 | 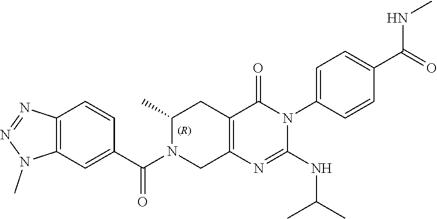 |
| C495 | 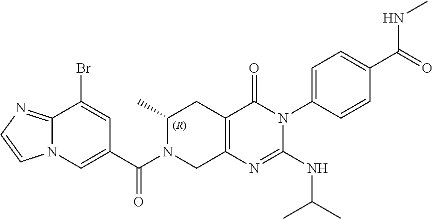 |
| C496 | 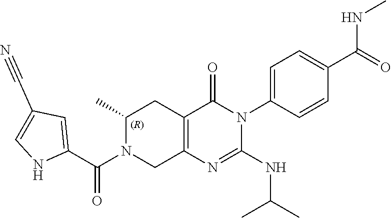 |
| C509 | 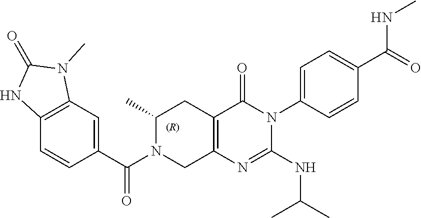 |

### Procedure AZ

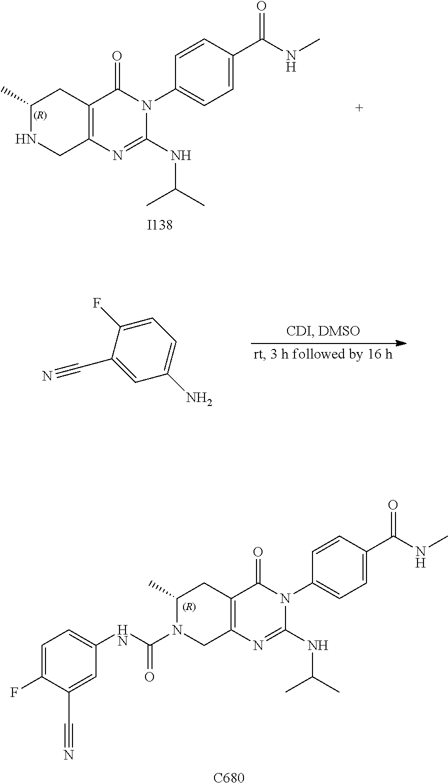

CDI (154 mg, 0.95 mmol) was added to a solution of 5-amino-2-fluorobenzonitrile (96 mg, 0.71 mmol) in DMSO (3 mL) at room temperature and the solution was stirred for 3 h. Then the solution was added to a mixture of (R)-4-(2-(isopropylamino)-6-methyl-4-oxo-5,6,7,8-tetrahydropyrido[3,4-d]pyrimidin-3(4H)-yl)-N-methylbenzamide I138 (202 mg, 0.472 mmol) in DMSO (2 mL), 1The reaction mixture was stirred for 16 h. The solvent was removed and a purification was performed via Prep HPLC (Stationary phase: RP XBridge Prep C18 OBD-10 μm, 50×150 mm, Mobile phase: 0.25% NH₄HCO₃ solution in water, CH₃CN). The product was recrystallized with a mixture of EtOH (4 mL) and water (5 mL) to afford the title compound C680 (64 mg, yield 26%) as a white powder.

### Procedure BA

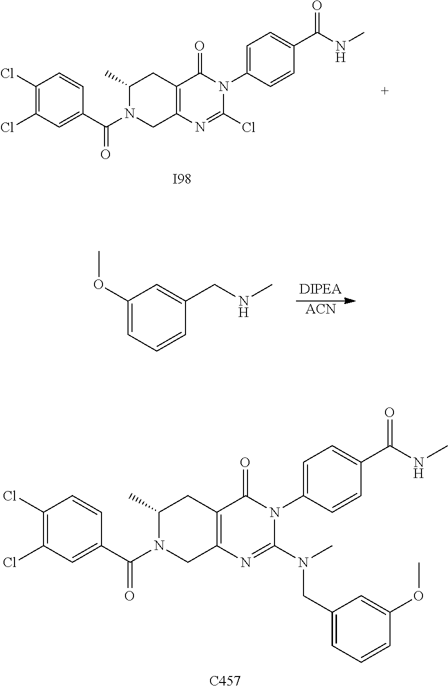

To a solution of (R)-4-(2-chloro-7-(3,4-dichlorobenzoyl)-6-methyl-4-oxo-5,6,7,8-tetrahydropyrido[3,4-d]pyrimidin-3(4H)-yl)-N-methylbenzamide 198 (150 mg, 0.29 mmol) in ACN (1.5 mL) was added DIPEA (153 μL, 0.89 mmol) and 1-(3-methoxy-phenyl)-N-methylmethanamine (89 mg, 0.60 mmol) and the μ-wave tube was sealed in a N₂-atm. The reaction mixture was heated at 80° C. for 16 h. The reaction mixture was concentrated and the crude was purified by column chromatography (DCM/MeOH 100/0 to 95/5). The product fractions were collected, concentrated and suspended in DIPE/ACN to afford the title compound C457 (90 mg, yield 49%) as a white solid.

In analogous manner, the compounds below were synthetized starting from intermediate I98 and appropriate amine.

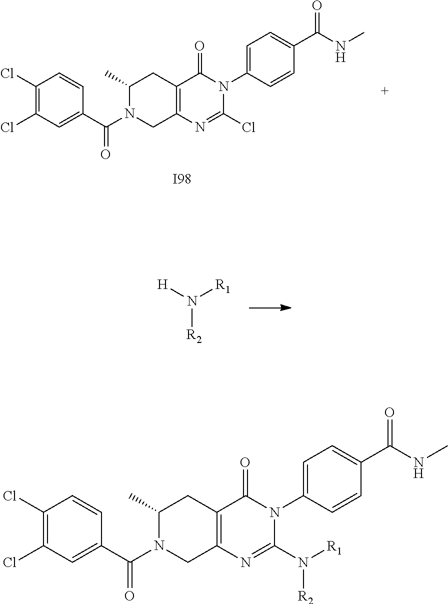

| Cmpd no | Structure |
|---|---|
| C372 | 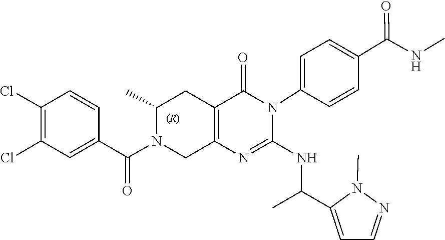 |
| C373 | 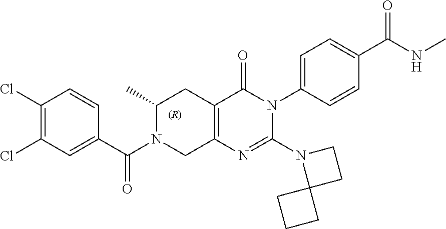 |
| C378 | 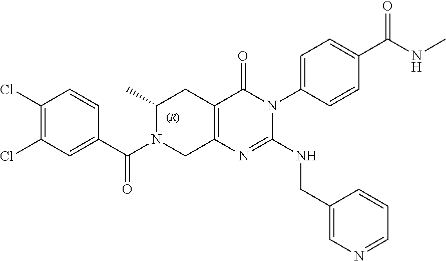 |
| C380 | 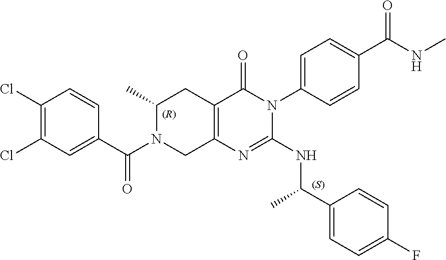 |
| C381 | 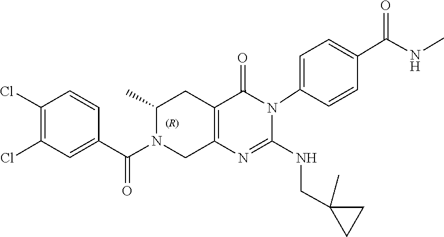 |
| C390 | 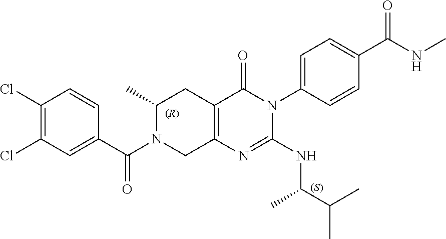 |
| C396 | 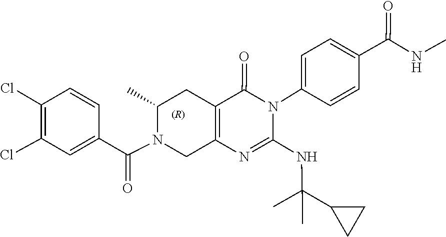 |
| C407 | 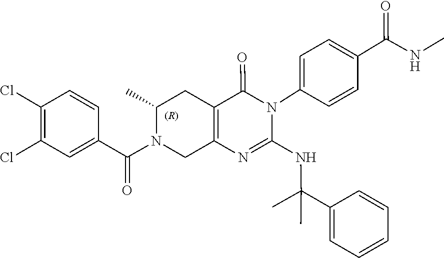 |
| C408 | 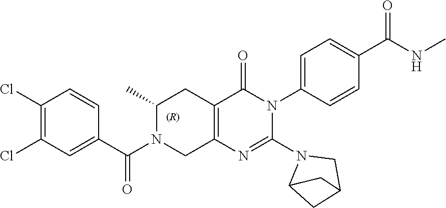 |
| C412 | 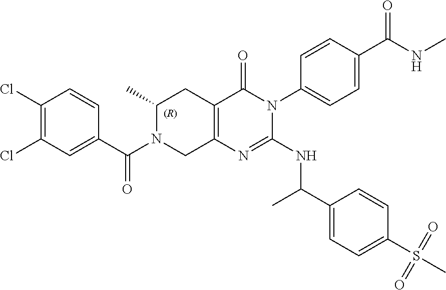 |
| C413 | 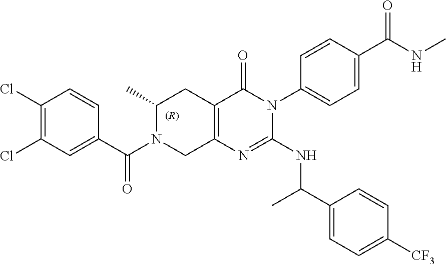 |
| C423^{x} | 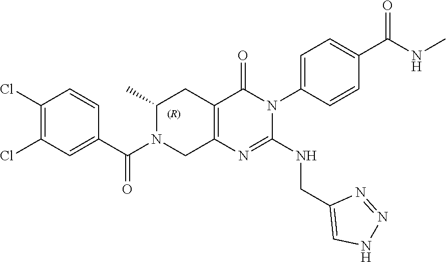 |
| C431^{x} | 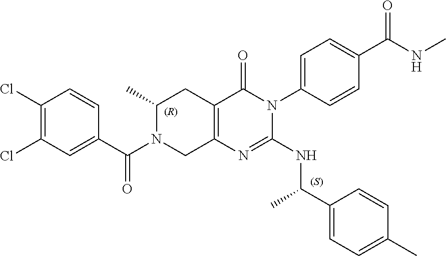 |
| C433^{x} | 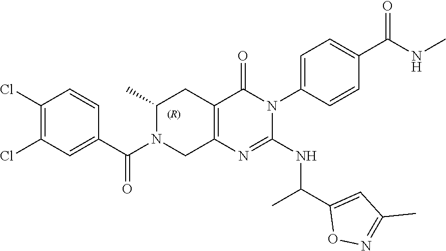 |
| C434^{x} | 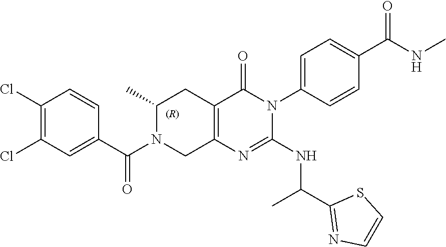 |
| C437^{x} | 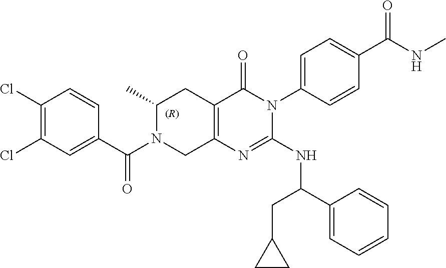 |
| C438^{x} | 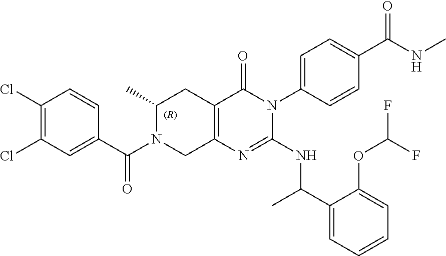 |
| C441 | 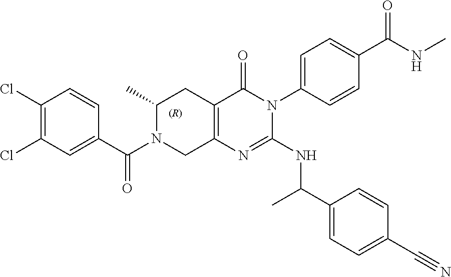 |
| C443^{x} | 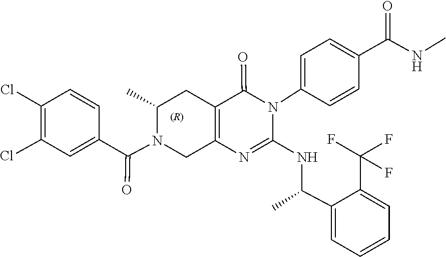 |
| C444 | 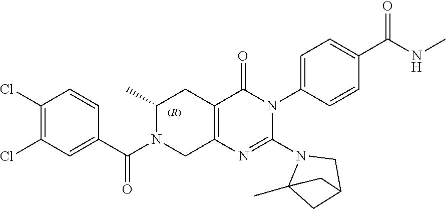 |
| C445^{x} | 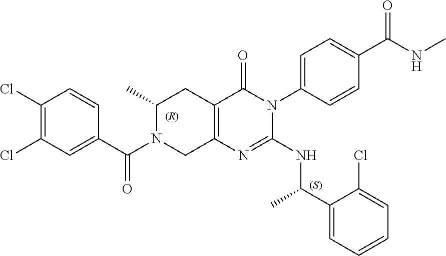 |
| C451 | 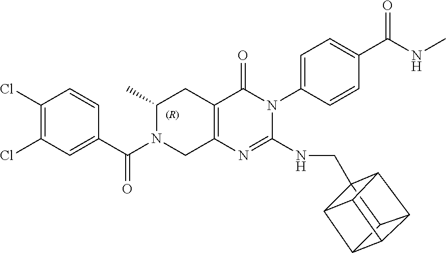 |
| C452 | 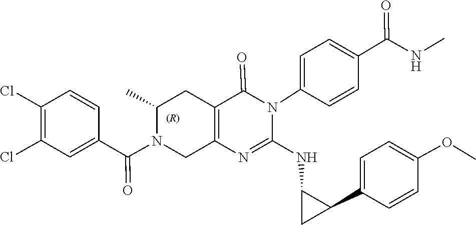 |
| C457 | 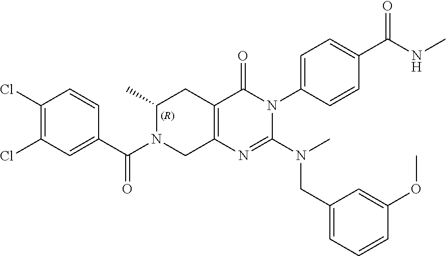 |
| C459 | 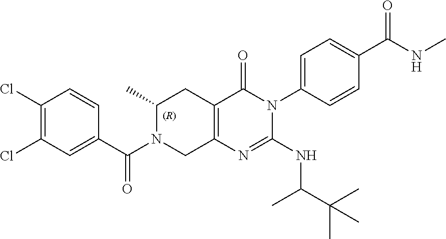 |
| C461 | 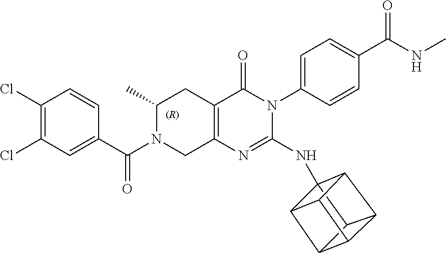 |
| C609 | 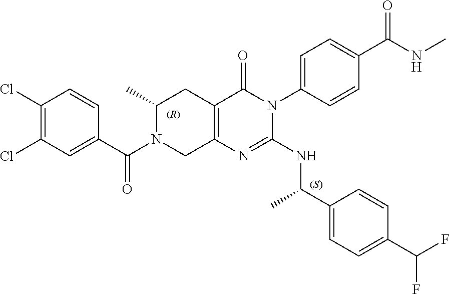 |
| C621 | 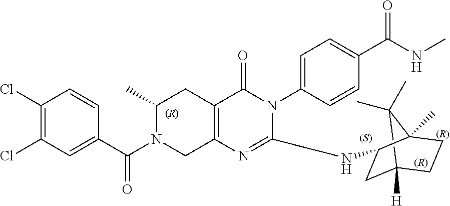 |
| C633 | 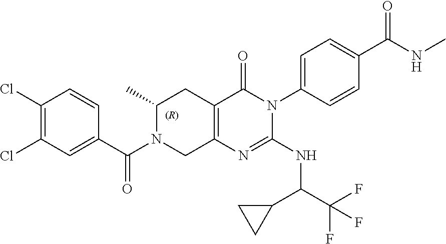 |
| C662^{x} | 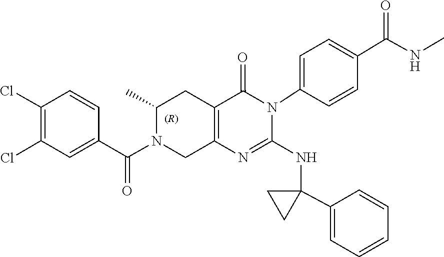 |
| C375 | 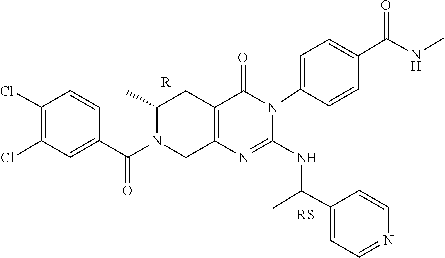 |
| C377 | 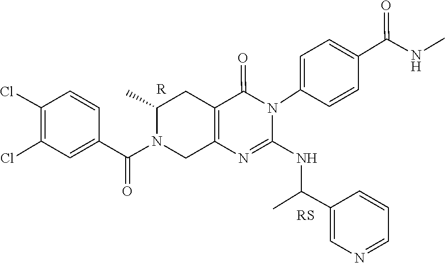 |
| C394 | 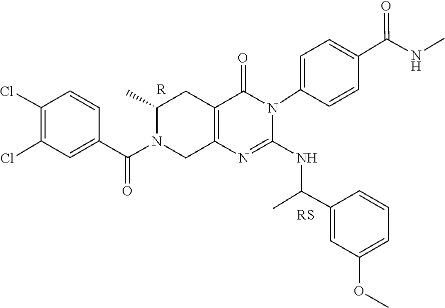 |
| C401 | 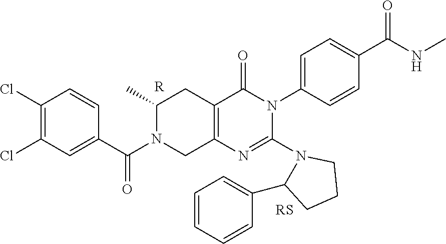 |
| C402 | 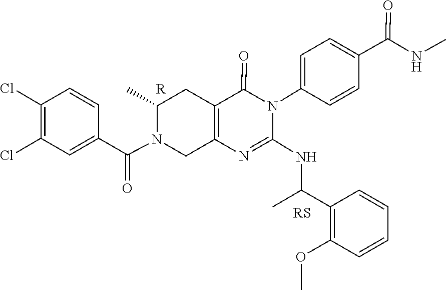 |
| C406 | 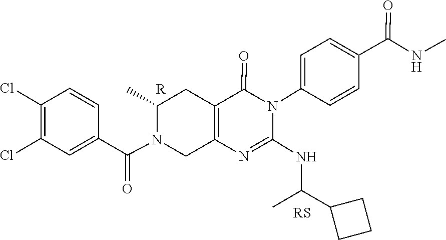 |
| C411 | 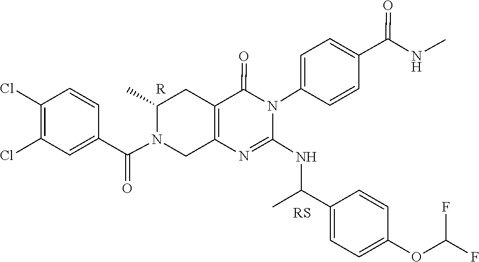 |
| C414 | 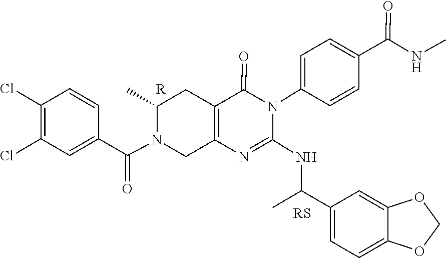 |
| C415 | 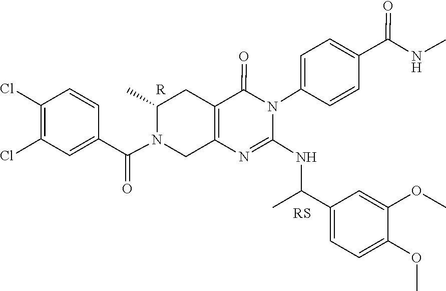 |
| C416 | 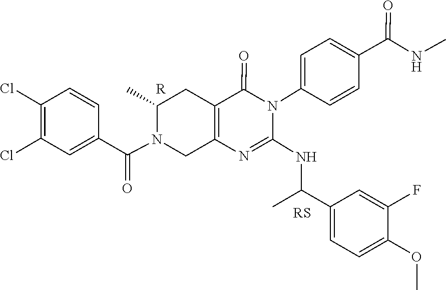 |
| C417 | 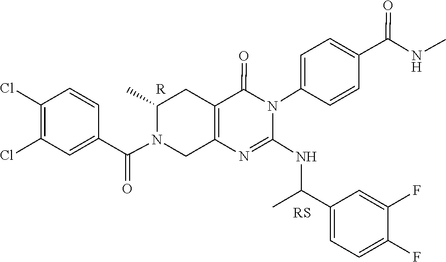 |
| C422 | 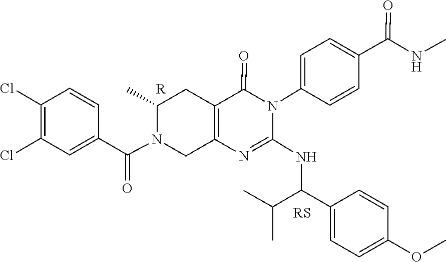 |
| C435 | 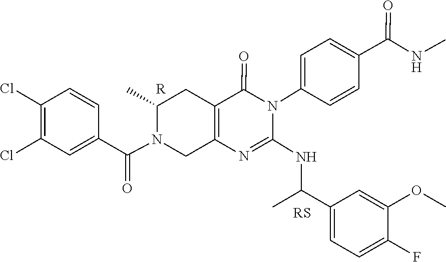 |
| C436 | 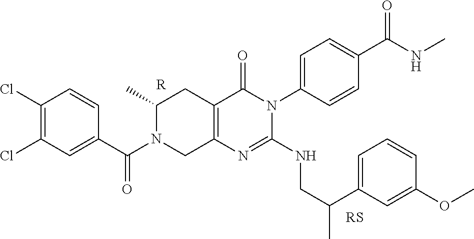 |
| C439^{x} | 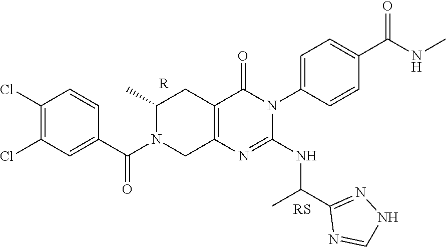 |
| C440^{x} | 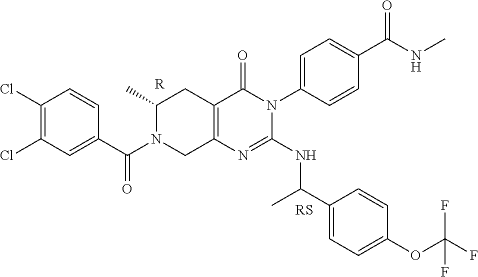 |
| C449 | 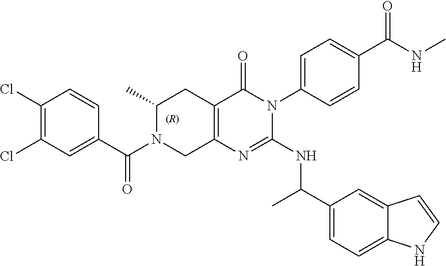 |
| C450 | 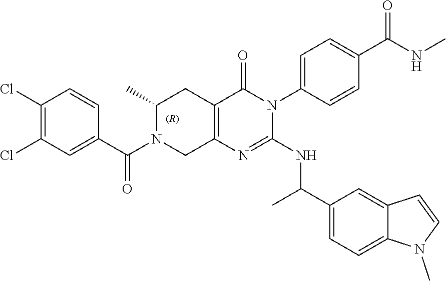 |
| C526^{x} | 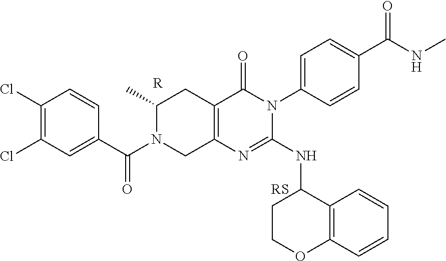 |
| C527^{x} | 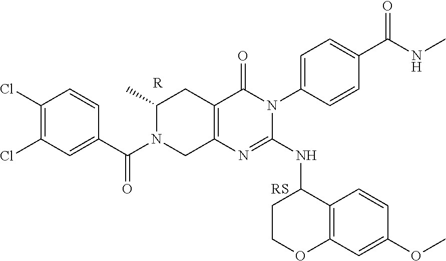 |
| C529 | 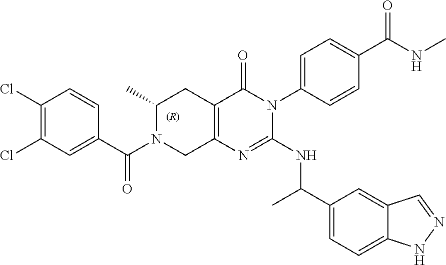 |
| C530 | 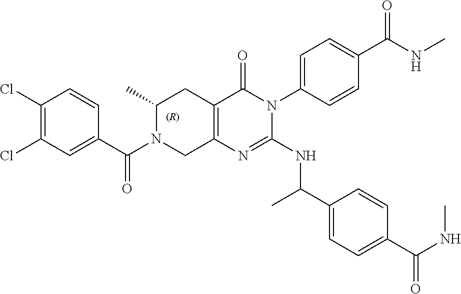 |
| C608 | 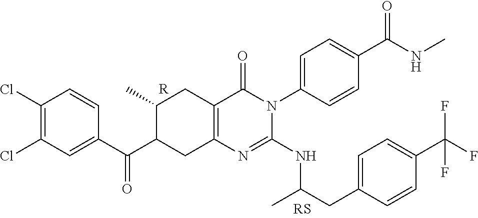 |
| C631 | 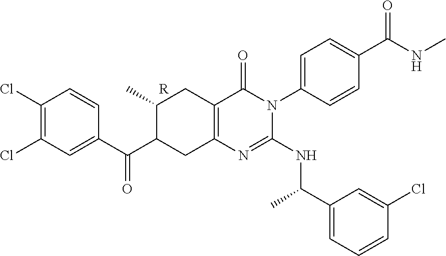 |
| C647^{x} | 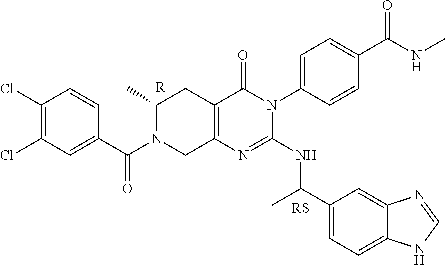 |
| C652^{x} | 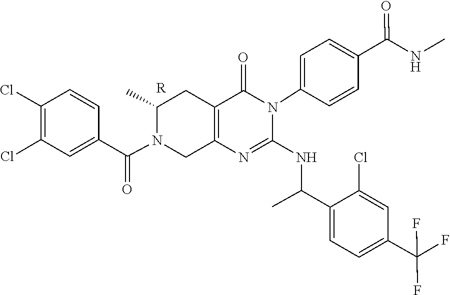 |
| C659 | 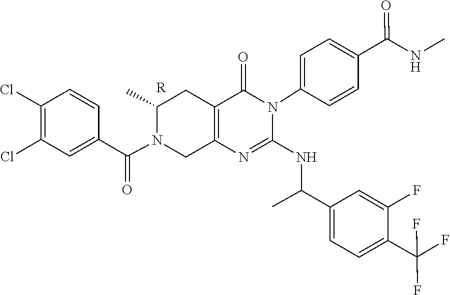 |
| C660^{x} | 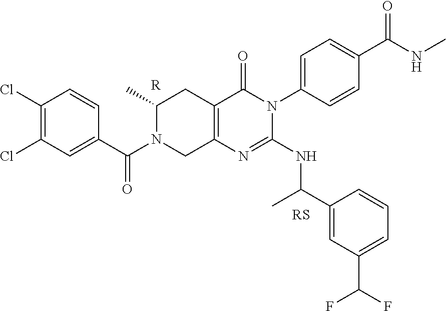 |
| C663^{xΩ} | 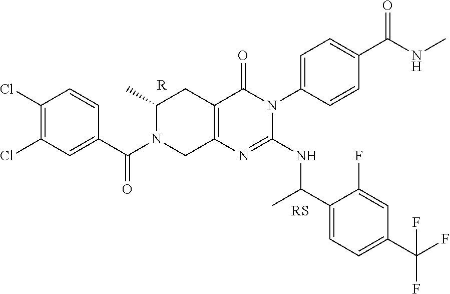 |
| C664^{x} | 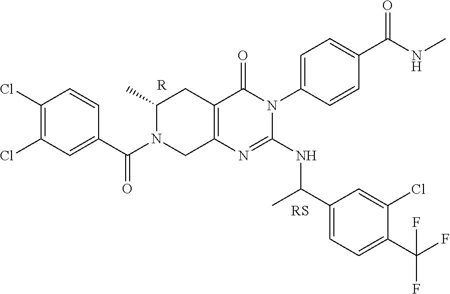 |
| C689 | 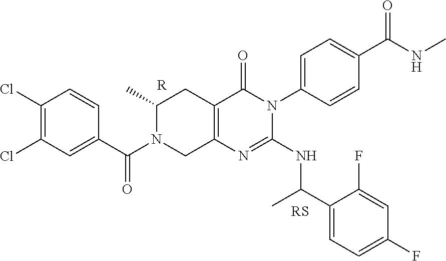 |
| C697 | 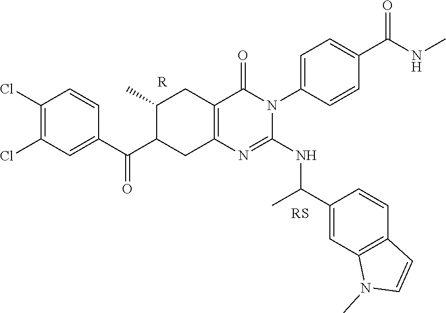 |

^{x}Et₃N used as base
^{Ω}C657 was obtained as by product due to contamination of 1-(2-fluoro-4-(trifluoromethyl)phenyl)ethan-1-amine with diethyl amine.

### SFC Separation

The compounds below have been purified by preparative SFC following one of the methods described in the general information “Separation of rotamers, diastereoisomers and enantiomers”

| Final compound | | Origin |
|---|---|---|
| 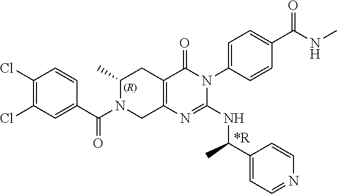 | 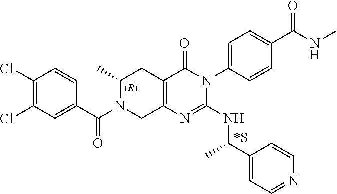 | C375 |
| C375a | 375b | |
| 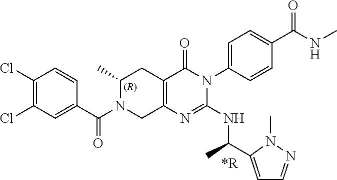 | 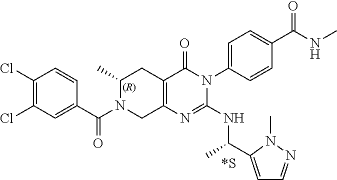 | C372 |
| C372a | 72514065 | |
| 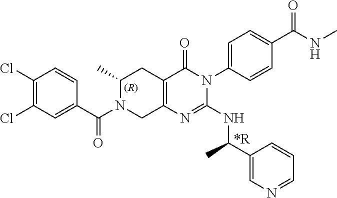 | 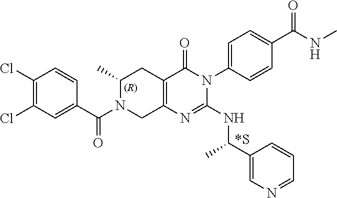 | C3777 |
| C377a | C377b | |
| 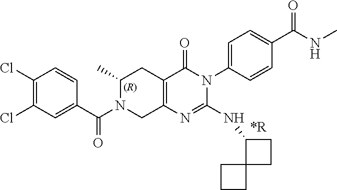 | 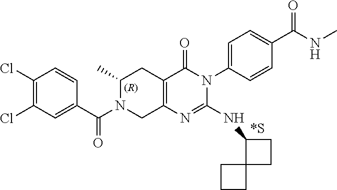 | C346 |
| C346a | C346a | |
| 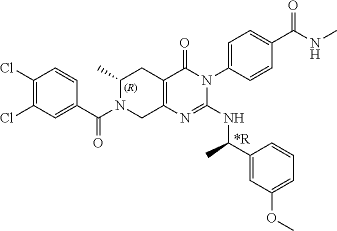 | 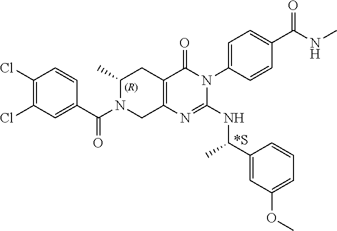 | C394 |
| C394a | C394b | |
| 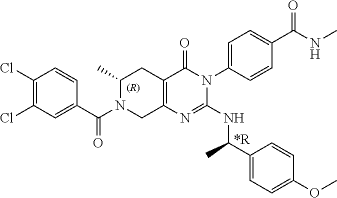 | 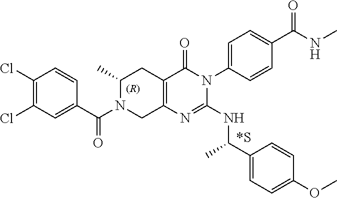 | C347 |
| C347a | C347b | |
| 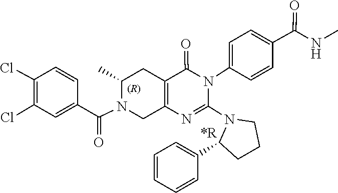 | 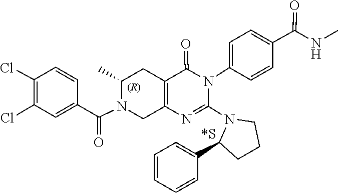 | C401 |
| C401a | C401b | |
| 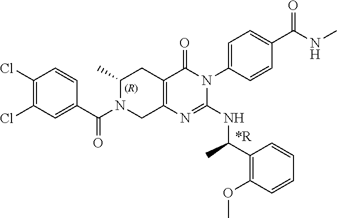 | 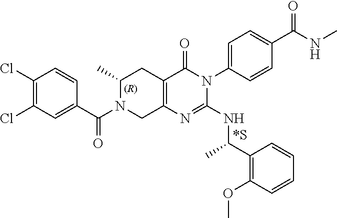 | C402 |
| C402a | C402b | |
| 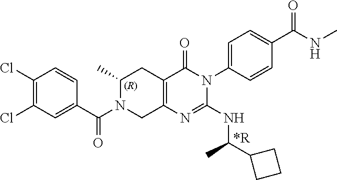 | 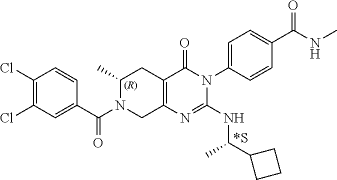 | C406 |
| C406a | C406b | |
| 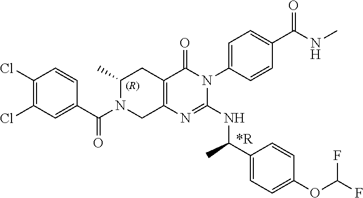 | 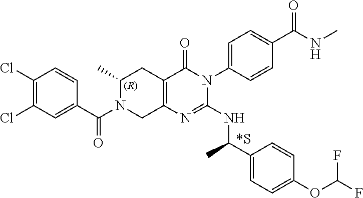 | C411 |
| C411a | C411b | |
| 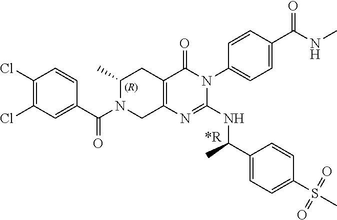 | 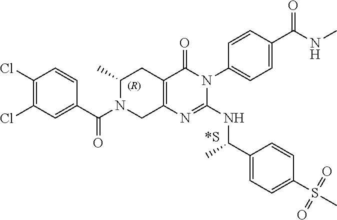 | C412 |
| C412a | C412b | |
| 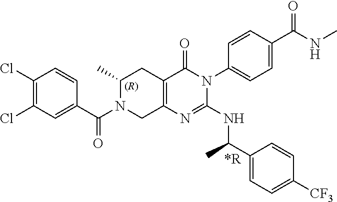 | 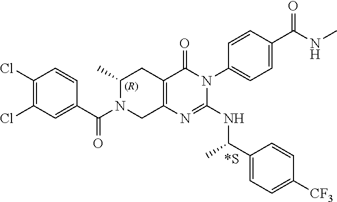 | C413 |
| C413a^{Δ} | C413b | |
| 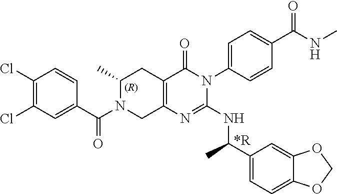 | 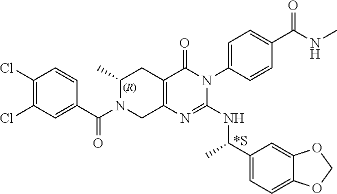 | C414 |
| C414a | C414b | |
| 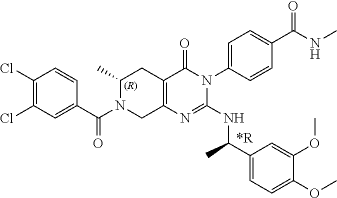 | 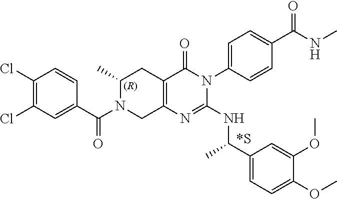 | C415 |
| C415a | C415b | |
| 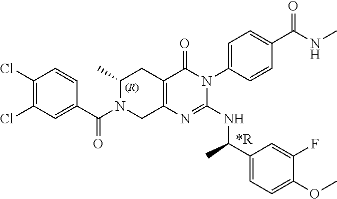 | 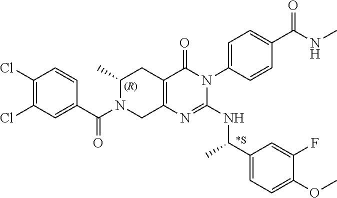 | C416b |
| C416a | C416b | |
| 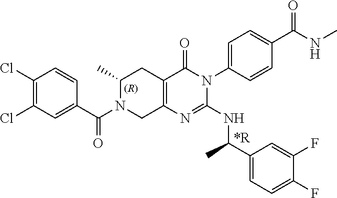 | 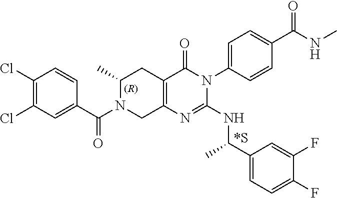 | C417 |
| C417a | C417b | |
| 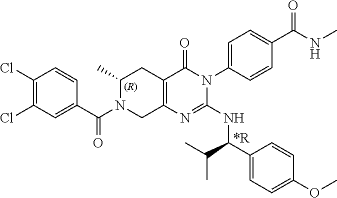 | 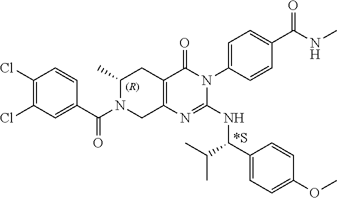 | C422 |
| C422a | C422b | |
| 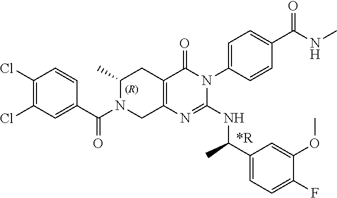 | 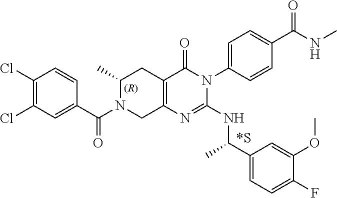 | C435 |
| C435a | C435b | |
| 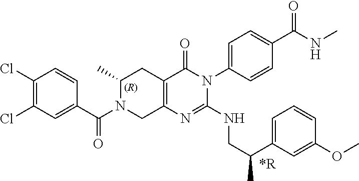 | 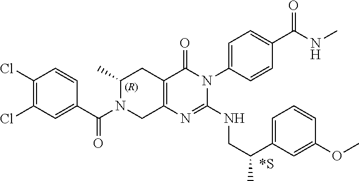 | C436 |
| C436a | C436b | |
| 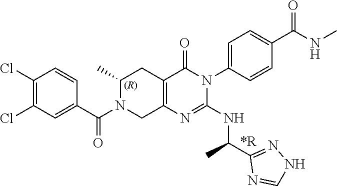 | 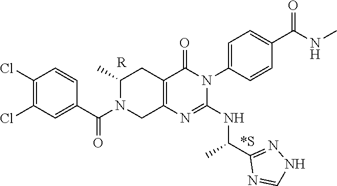 | C439 |
| C439a | C439b | |
| 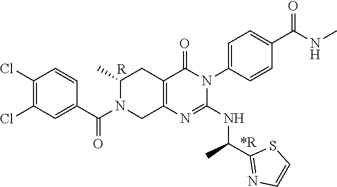 | 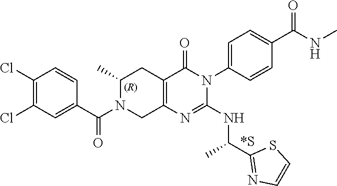 | C434 |
| C434a | C434b | |
| 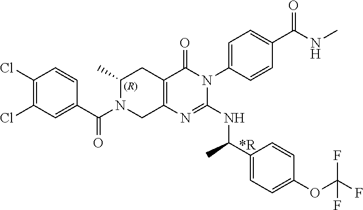 | 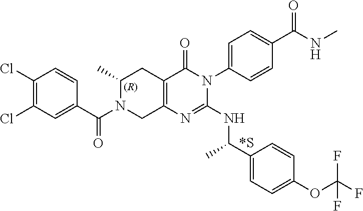 | C440 |
| C440a | C440b | |
| 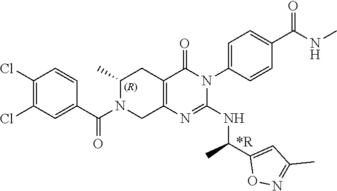 | 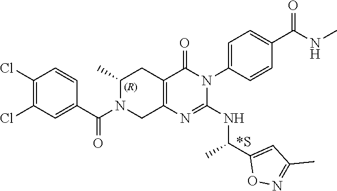 | C433 |
| C433a | C433b | |
| 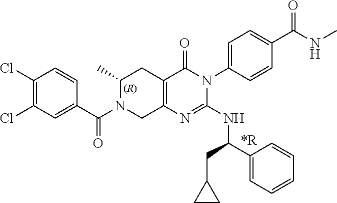 | 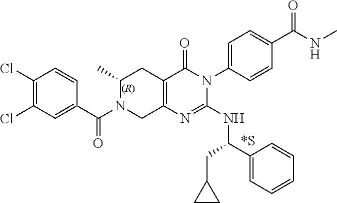 | C437 |
| C437a | C437b | |
| 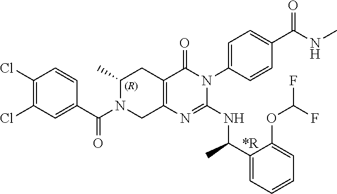 | 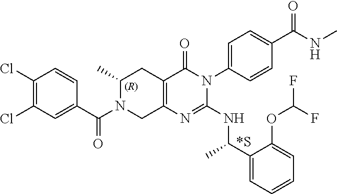 | C438 |
| C438a | C438b | |
| 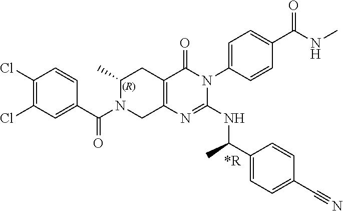 | 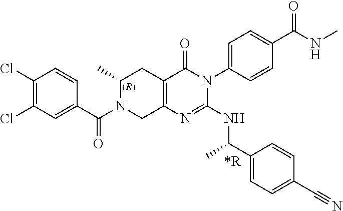 | C441 |
| C441a | C441b | |
| 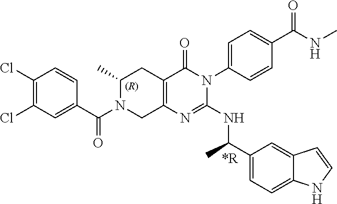 | 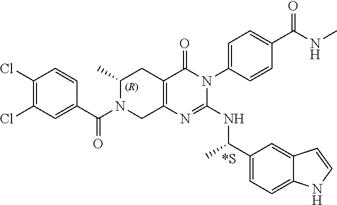 | C449 |
| C449a | C449b | |
| 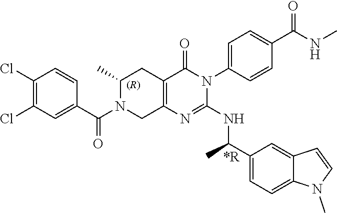 | 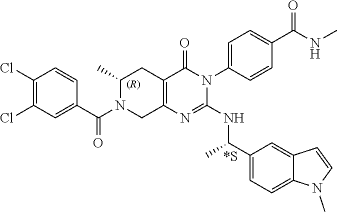 | C450 |
| C450a | C450b | |
| 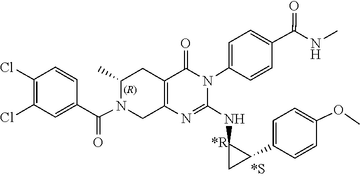 | 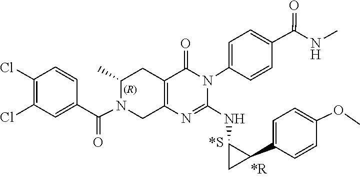 | C452 |
| C452a | C452b | |
| 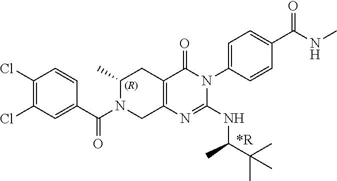 | 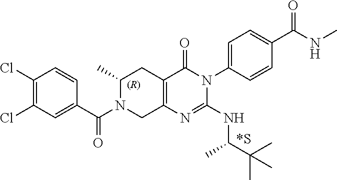 | C459 |
| C459a | C459b | |
| 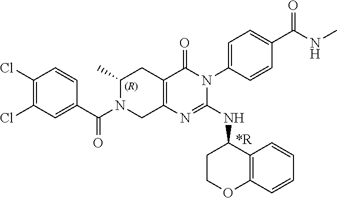 | 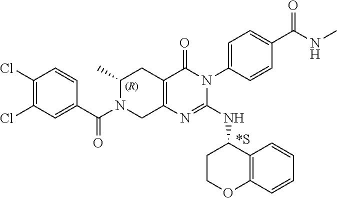 | C526a |
| C526a | C526b | |
| 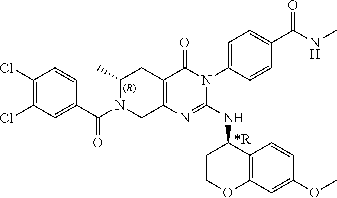 | 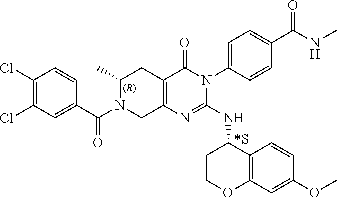 | C527 |
| C527a | C527b | |
| 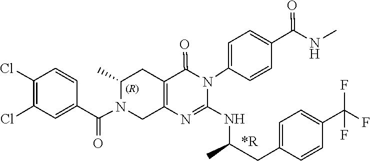 | 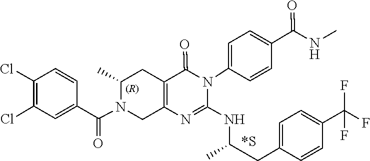 | C608 |
| C608a | C608b | |
| 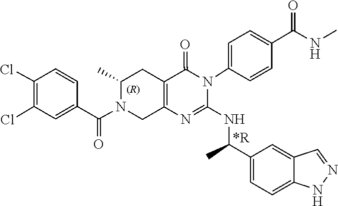 | 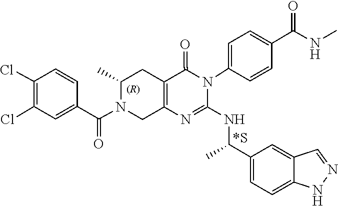 | C529 |
| C529a | C529a | |
| 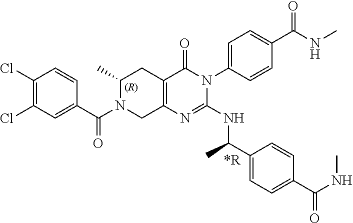 | 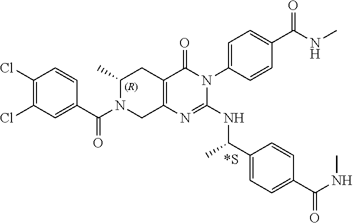 | C530 |
| C530a | C530b | |
| 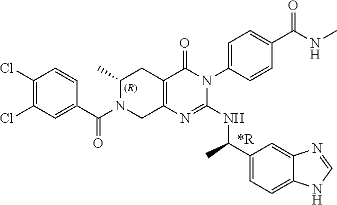 | 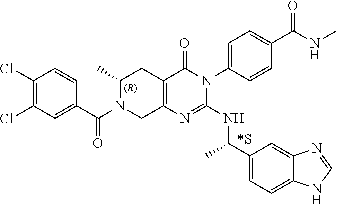 | C647 |
| C647a | C647b | |
| 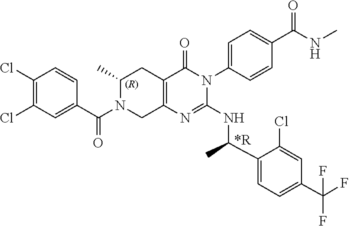 | 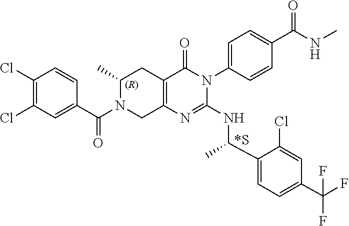 | C652 |
| C652a | C652b | |
| 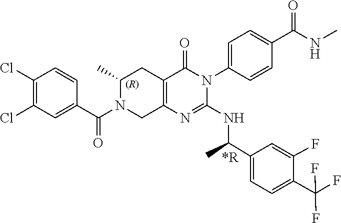 | 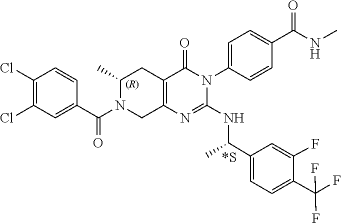 | C659 |
| C659a | C659b | |
| 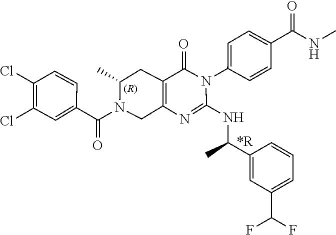 | 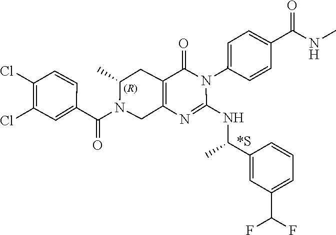 | C660 |
| C660a | C660b | |
| 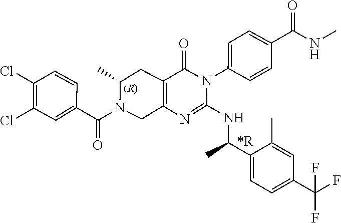 | 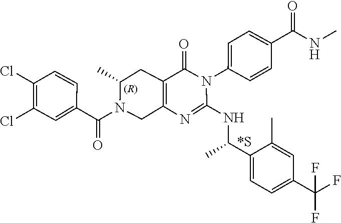 | C663 |
| C663a | C663b | |
| 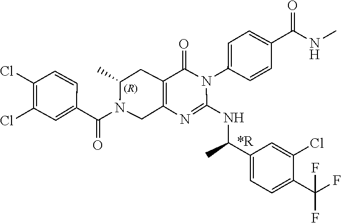 | 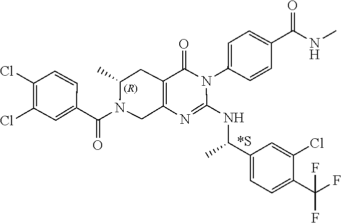 | C664 |
| C664a | C664b | |
| 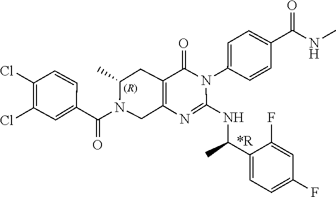 | 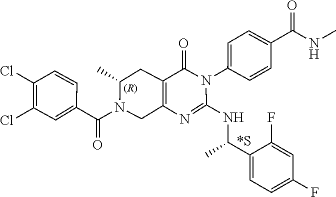 | C689 |
| C689a | C689b | |
| 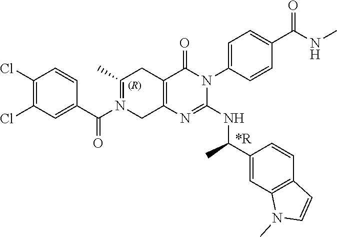 | 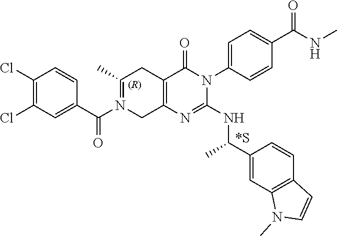 | C697 |
| C697a | C697b | |

^{Δ}Synthesis performed with enantiomeric pure (S)-1-[4-(trifluoromethyl)phenyl]-ethylamine demonstrate that C413a has the absolute S configuration.

### Procedure BB

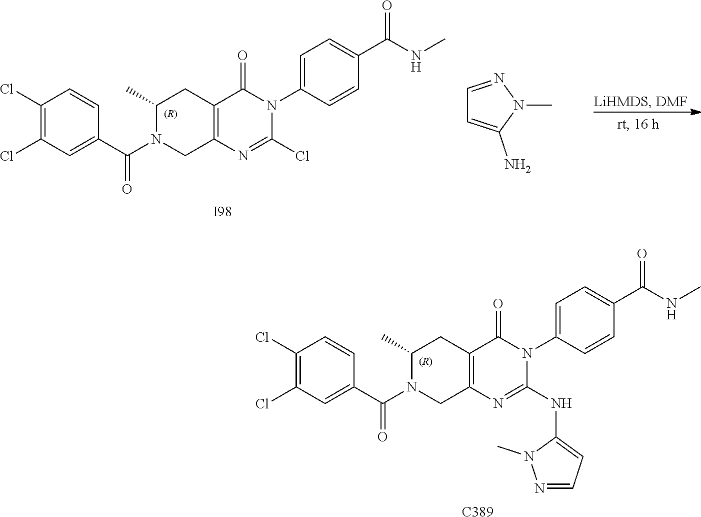

A reaction tube was charged with R-4-(2-chloro-7-(3,4-dichlorobenzoyl)-6-methyl-4-oxo-5,6,7,8-tetrahydropyrido[3,4-d]pyrimidin-3(4H)-yl)-N-methylbenzamide 198 (469 mg, 0.93 mmol), 1-methyl-1H-pyrazol-5-amine (108 mg, 1.11 mmol) in DMF (5 mL). LiHMDS (2.187 mL, 1.06 M in THF, 2.32 mmol) was added dropwise to the mixture at room temperature. The mixture was stirred for 16 h. The mixture was poured out in ice water and neutralized with 1 N HCl solution. The mixture was extracted with Me-THF(2×) and the organic layers were combined, dried over MgSO₄, filtered and concentrated in vacuo. The residue was purified by column chromatography using (DCM/(DCM:MeOH/NH₃ (9:1) from 100/0 to 0/100 in DCM. The product fractions were collected and concentrated in vacuo. The product was triturated in DIPE, filtered off and dried under vacuum to become the title compound C389 (325 mg, yield 62%).

The compounds listed below were synthetized in an analogous manner, starting from intermediate I98 and appropriate amine.

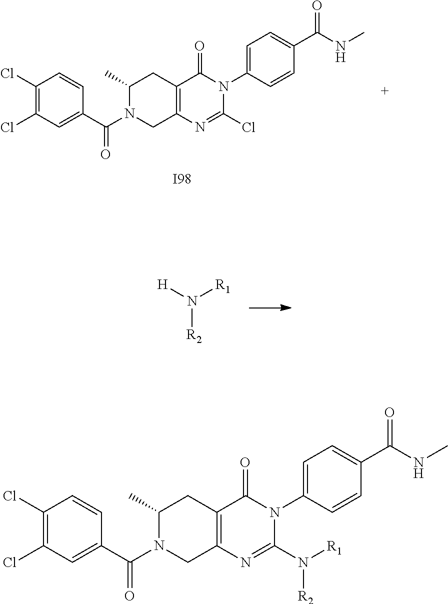

| Cmpd | |
|---|---|
| no | Structure |
| C389 | 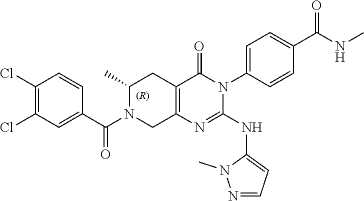 |
| C421 | 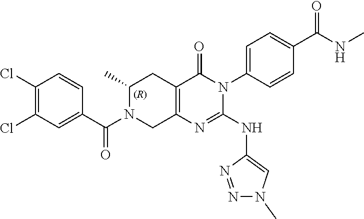 |

### Procedure BC

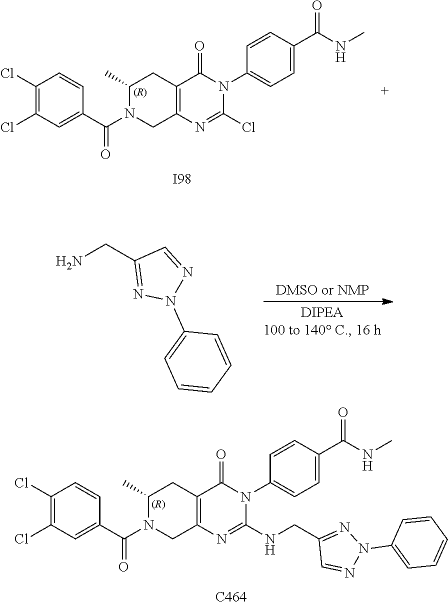

(2-phenyl-2H-1,2,3-triazol-4-yl)methanamine (261 mg, 1.5 mmol) and R-4-(2-chloro-7-(3,4-dichlorobenzoyl)-6-methyl-4-oxo-5,6, 7, 8-tetrahydropyrido[3,4-d]pyrimidin-3(4H)-yl)-N-methylbenzamide 198 (505 mg, 1 mmol) were placed in a vial and the mixture was dissolved in dry NMP (1 mL). Then DIPEA♦ (0.862 mL, 0.75 g/mL, 5 mmol) was added. The reaction mixture was heated with stirring for 16 h at 140° C. After cooling to ambient temperature, the mixture was evaporated under high vacuum. The residue was dissolved in DMSO, and the solution was subjected to HPLC purification♦♦ to obtain the title compound C464 (399 mg, yield 62%).

♦In case of using a salt of the reagent, an additional amount of DIPEA was added to the reaction mixture to transfer the amine to the base form.

♦♦The purification was performed using Agilent 1260 Infinity systems equipped with DAD and mass-detector. Waters Sunfire C18 OBD Prep Column, 100 Å, 5 μm, 19 mm×100 mm with SunFire C18 Prep Guard Cartridge, 100 Å, 10 μm, 19 mm×10 mm was used. Deionized Water (phase A) and HPLC-grade Methanol (phase B) were used as an eluent. In some cases, ammonia or TFA was used as an additive to improve the separation of the products. In these cases, free bases and TFA salts of the products were formed respectively.

The compounds listed below were synthetized in an analogous manner, starting from intermediate I98 and appropriate amine.

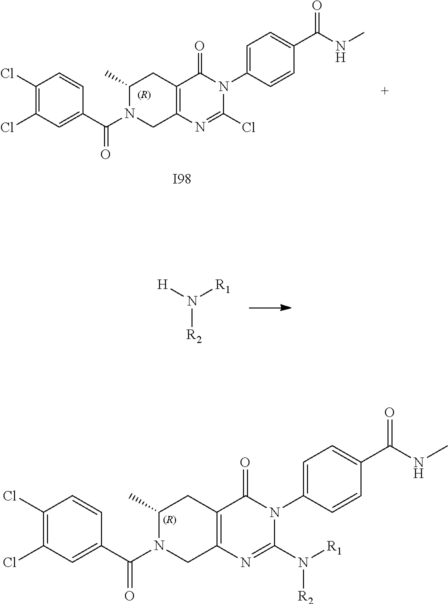

| Cmpd | |
|---|---|
| no | Structure |
| C464 | 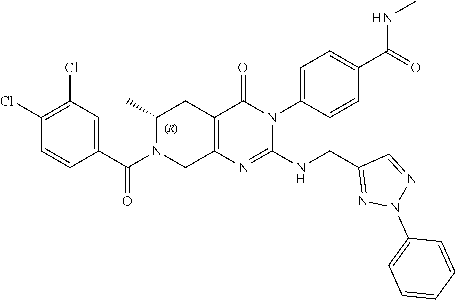 |
| C465 | 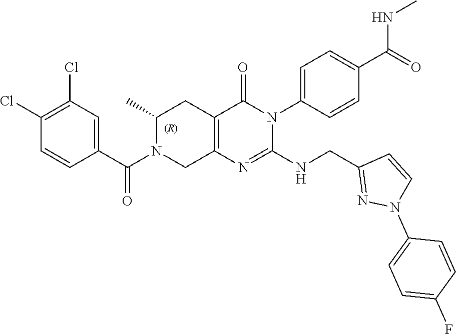 |
| C466 | 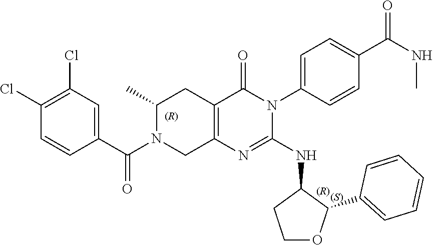 |
| C467 | 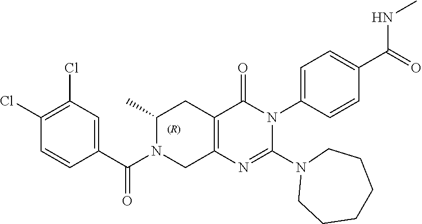 |
| C468 | 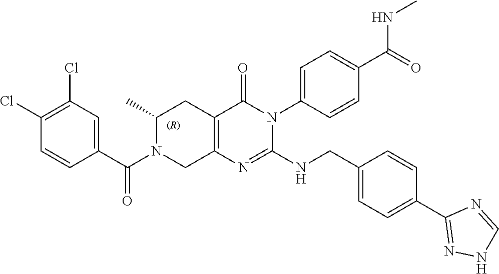 |
| C469 | 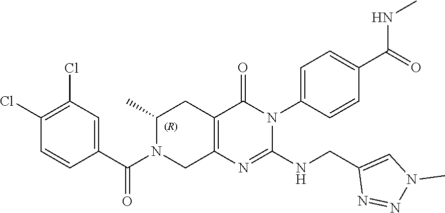 |
| C470 | 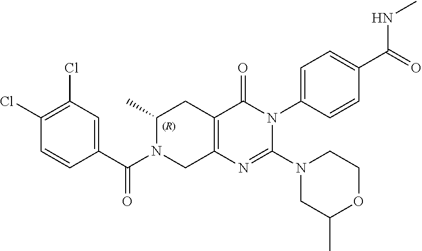 |
| C479 | 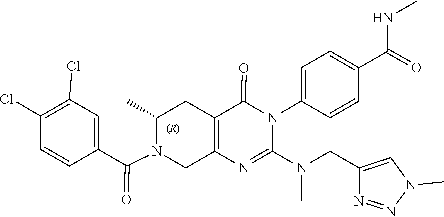 |
| C480 | 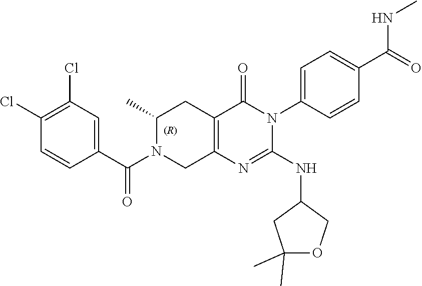 |
| C481 | 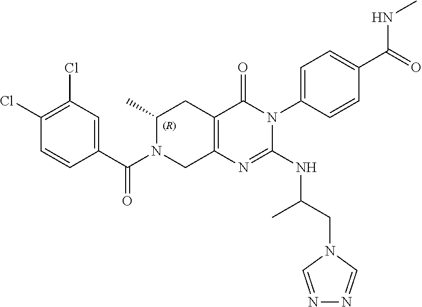 |
| C482 | 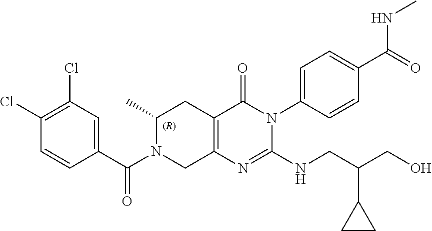 |
| C483 | 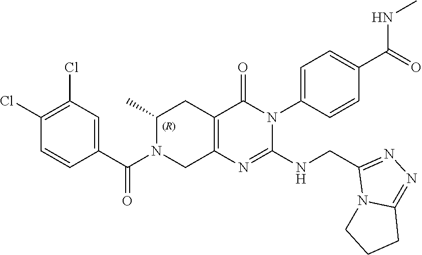 |
| C484 | 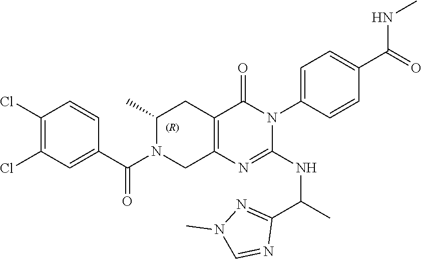 |
| C485 | 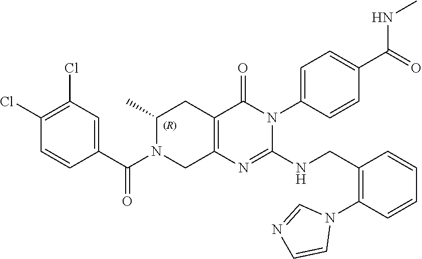 |
| C486 | 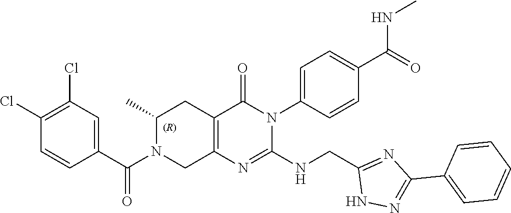 |
| C487 | 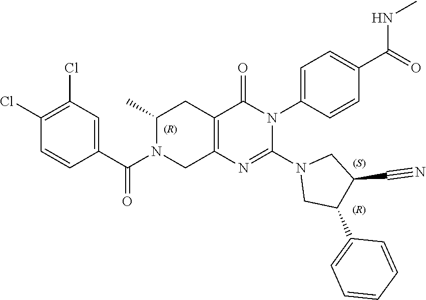 |
| C488 | 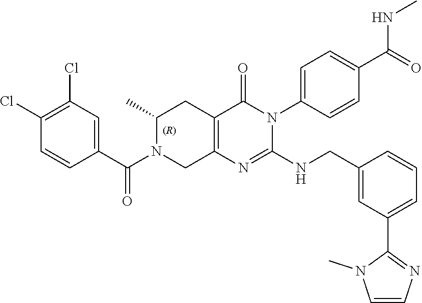 |
| C489 | 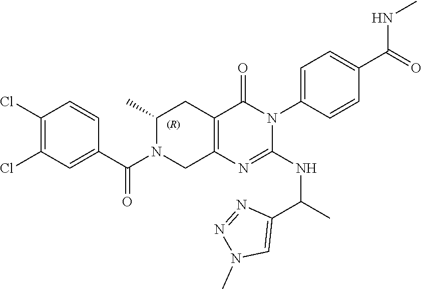 |
| C490 | 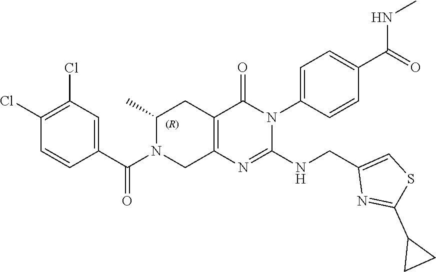 |
| C517 | 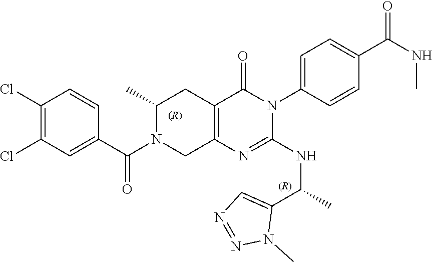 |
| C518 | 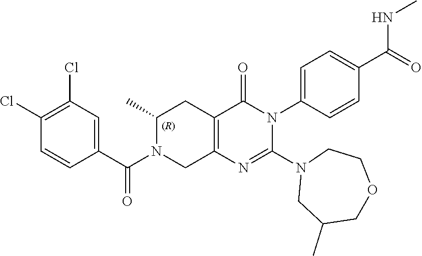 |
| C538 | 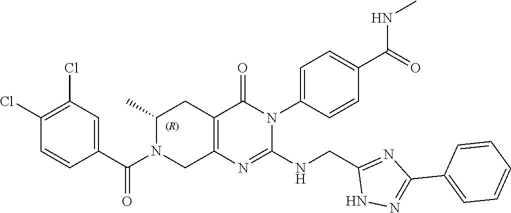 |
| C539 | 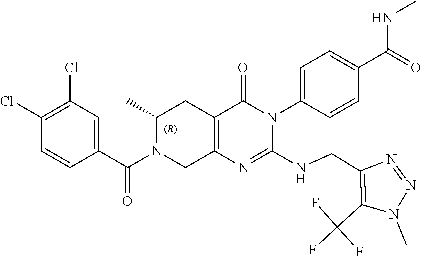 |
| C540 | 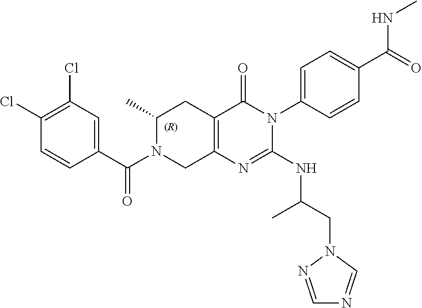 |
| C541 | 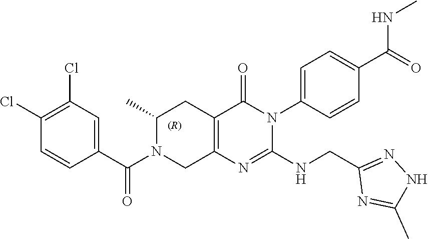 |
| C542 | 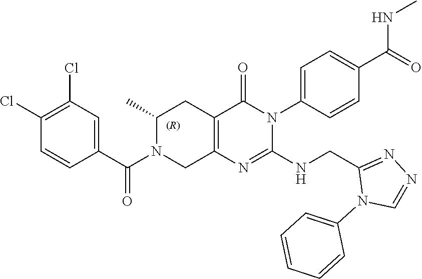 |
| C543 | 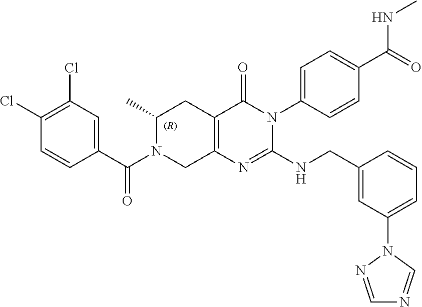 |
| C544 | 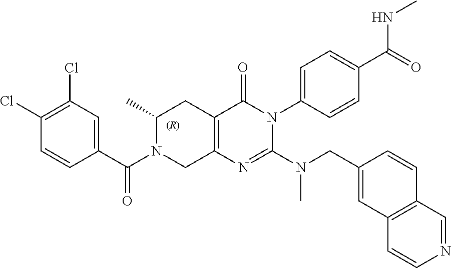 |
| C545 | 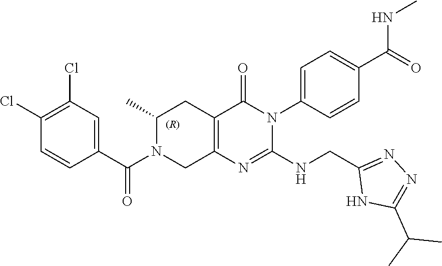 |
| C546 | 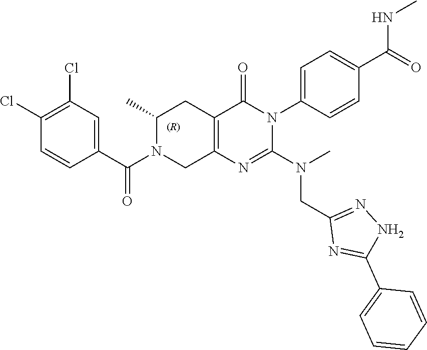 |
| C547 | 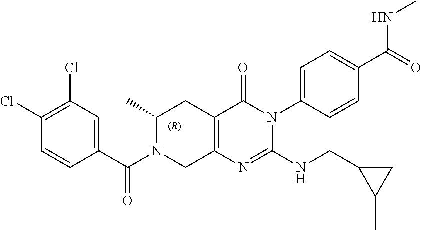 |
| C548 | 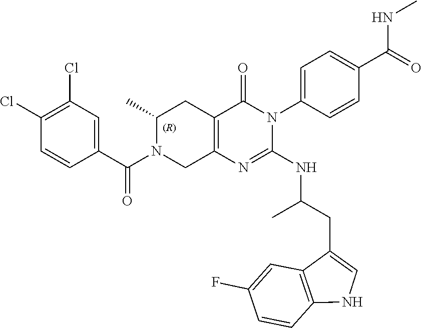 |
| C549 | 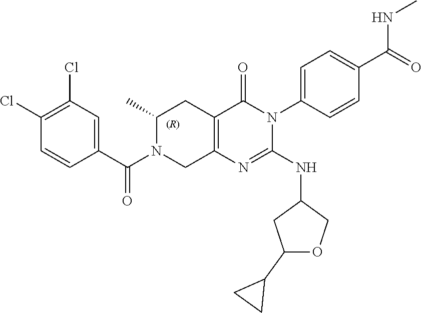 |
| C550 | 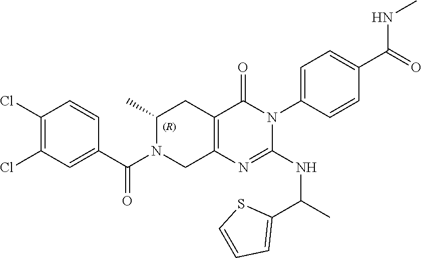 |
| C551 | 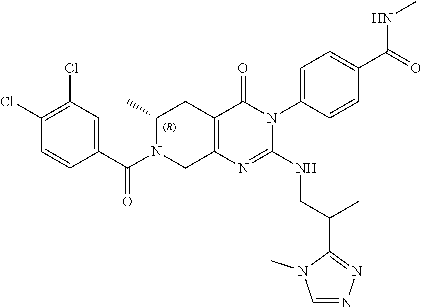 |
| C552 | 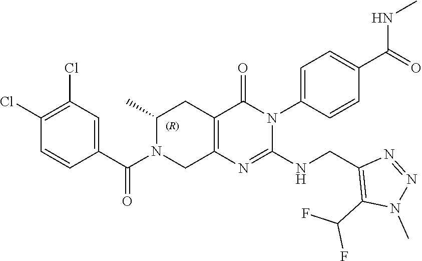 |
| C553 | 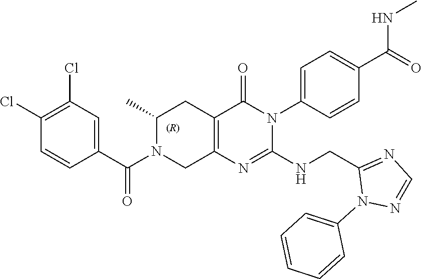 |
| C554 | 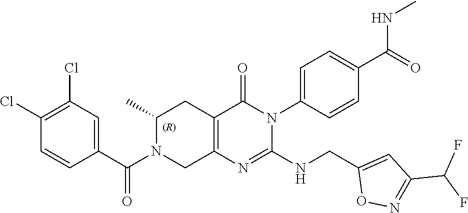 |
| C555 | 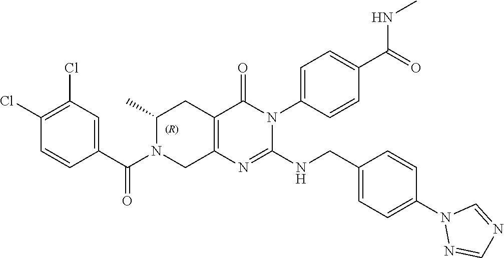 |
| C556 | 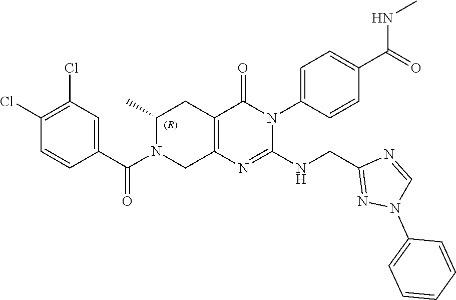 |
| C557 | 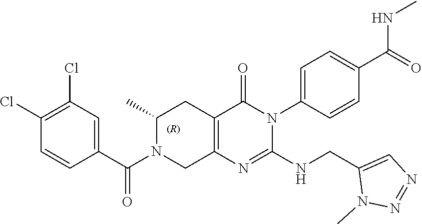 |
| C558 | 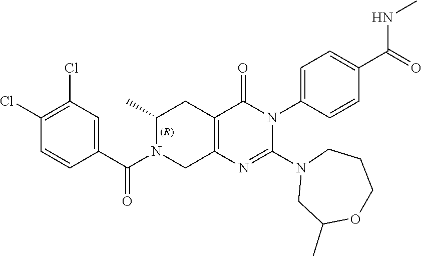 |
| C559 | 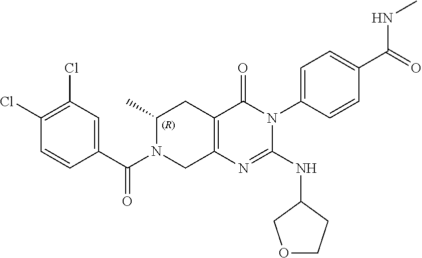 |
| C560 | 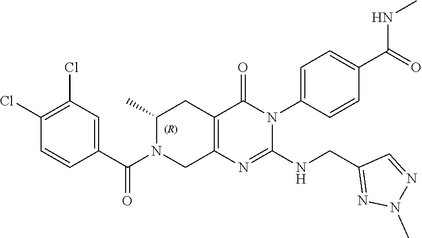 |
| C561 | 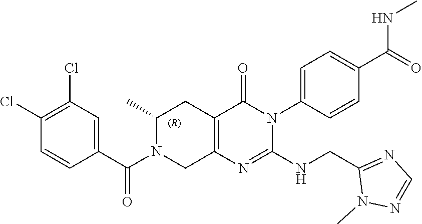 |
| C562 | 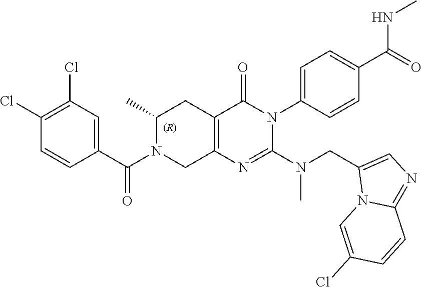 |
| C595 | 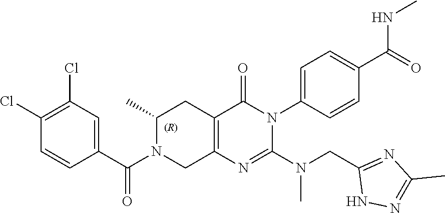 |
| C596 | 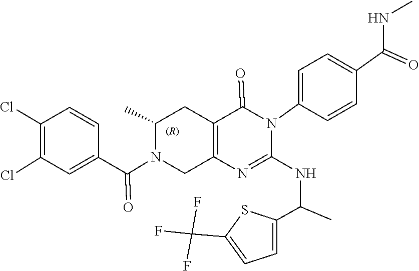 |
| C597 | 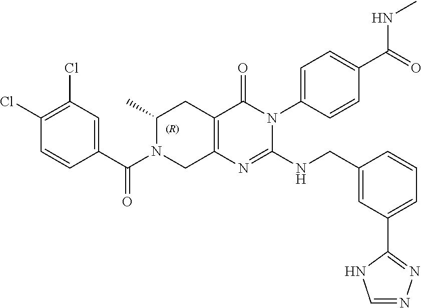 |
| C598 | 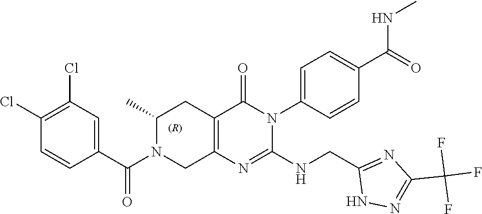 |
| C599 | 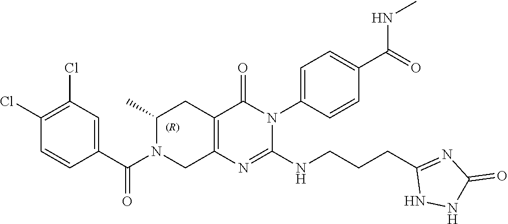 |
| C600 | 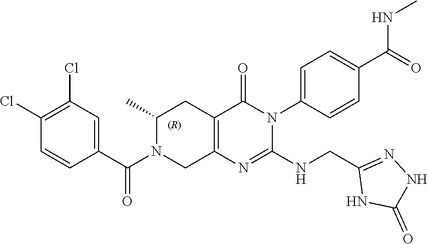 |
| C601 | 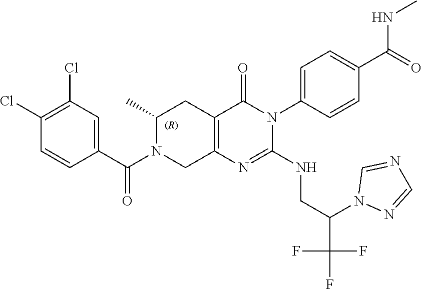 |
| C603 | 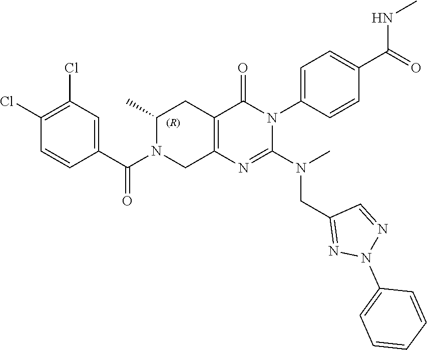 |
| C605 | 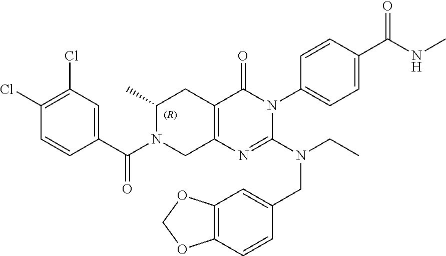 |

### Procedure BD

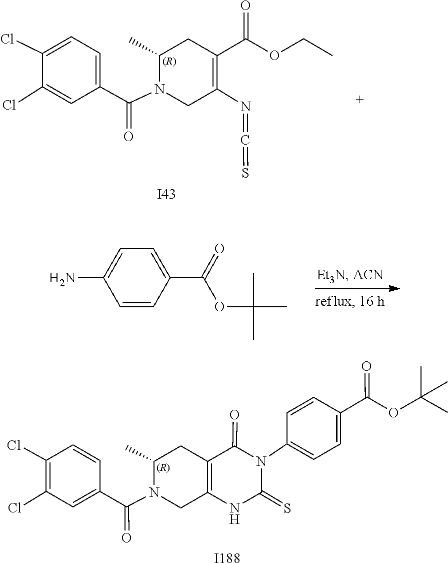

A flask was charged with ethyl R-1-(3,4-dichlorobenzoyl)-5-isothiocyanato-2-methyl-1,2,3,6-tetrahydropyridine-4-carboxylate (2 g, 5.01 mmol) 143, tert-butyl 4-aminobenzoate (1.26 g, 6.51 mmol), Et₃N (1.04 mL, 7.51 mmol) in dry CH₃CN (10 mL). The mixture was heated at reflux for 16 h. The mixture was cooled and concentrated in vacuo. The residue was triturated in DIPE/CH₃CN, filtered off and dried under vacuum to obtain the title compound tert-butyl R-4-(7-(3,4-dichlorobenzoyl)-6-methyl-4-oxo-2-thioxo-1,4,5,6,7,8-hexahydropyrido[3,4-d]pyrimidin-3(2H)-yl)benzoate I188 (2.5 g, yield 91%) as a pale yellow solid.

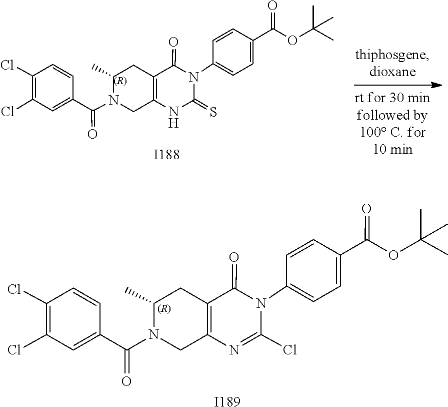

A reaction vial was charged with tert-butyl R-4-(7-(3,4-dichlorobenzoyl)-6-methyl-4-oxo-2-thioxo-1,4,5,6,7,8-hexahydropyrido[3,4-d]pyrimidin-3(2H)-yl)benzoate I188 (500 mg, 0.912 mmol) in dry 1,4-dioxane (4.7 mL). Thiophosgene (79 μL, 1.01 mmol) was added and the mixture was stirred at RT for 30 min. The mixture was then heated at 100° C. for 10 min. The mixture was concentrated in vacuo. The residue was purified by column chromatography on a 25 g SNAP cartridge in a Biotage system using a gradient from 0 till 100% EtOAc in Heptane over 12cv. The product fractions were collected and concentrated in vacuo to become tert-butyl R-4-(7-(3,4-dichlorobenzoyl)-6-methyl-4-oxo-2-thioxo-1,4,5,6,7,8-hexahydropyrido[3,4-d]-pyrimidin-3(2H)-yl)benzoate I189 (475 mg, yield 95%) as a yellow foam.

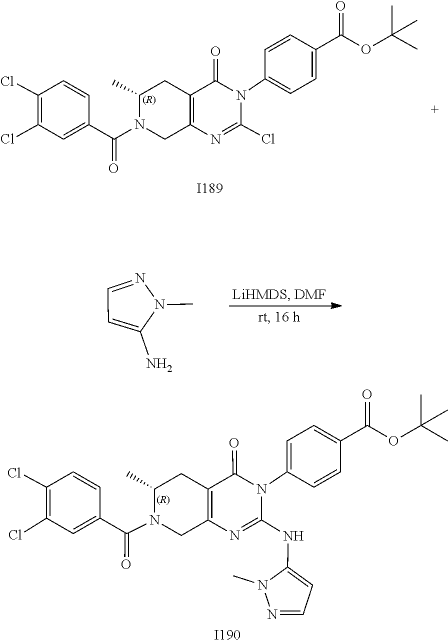

A reaction tube was charged with tert-butyl R-4-(7-(3,4-dichlorobenzoyl)-6-methyl-4-oxo-2-thioxo-1,4,5,6,7,8-hexahydropyrido[3,4-d]pyrimidin-3(2H)-yl)benzoate I189 (475 mg, 0.87 mmol), 1-methyl-1H-pyrazol-5-amine (100.8 mg, 1.04 mmol) in dry DMF (5.8 mL). LiHMDS (2.04 mL, 1.06 M in THF, 2.16 mmol) was added dropwise to the mixture at RT. The mixture was stirred at RT for 16 h. The mixture was poured out in sat. NH₄Cl solution. The mixture was extracted with Me-THF (2×) and the organic layers were combined, dried over MgSO₄, filtered and concentrated in vacuo. The residue was purified by column chromatography (heptane/EtOAC from 100/0 to 0/100) to afford the title compound tert-butyl R-4-(7-(3,4-dichlorobenzoyl)-6-methyl-2-((1-methyl-1H-pyrazol-5-yl)amino)-4-oxo-5,6,7,8-tetrahydropyrido[3,4-d]pyrimidin-3(4H)-yl)benzoate I190 (337 mg, yield 64%).

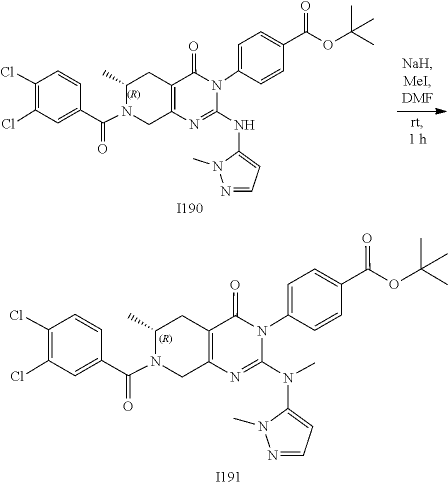

A reaction tube was charged with tert-butyl R-4-(7-(3,4-dichlorobenzoyl)-6-methyl-2-((1-methyl-1H-pyrazol-5-yl)amino)-4-oxo-5,6,7,8-tetrahydropyrido[3,4-d]pyrimidin-3(4H)-yl)benzoate I190 (337 mg, 0.553 mmol) in dry DMF (3 mL) under N₂. NaH (60% dispersion in mineral oil) (28.74 mg, 0.72 mmol) was added and the mixture was stirred at RT for 5 min. Mel (38 μL, 0.61 mmol) was added and the mixture was stirred at RT for 1 h. The mixture was concentrated in vacuo. The residue was purified by column chromatography DCM/[DCM-MeOH/NH₃(9-1)] from 100/0 to 0/100 to afford the title compound tert-butyl R-4-(7-(3,4-dichlorobenzoyl)-6-methyl-2-(methyl(1-methyl-1H-pyrazol-5-yl)amino)-4-oxo-5,6,7,8-tetrahydropyrido[3,4-d]pyrimidin-3(4H)-yl)benzoate I191 (147 mg, yield 43%).

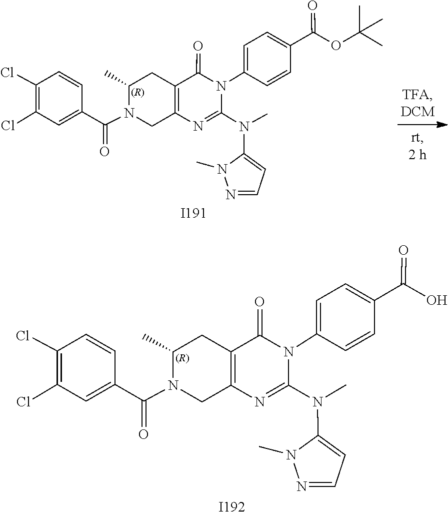

A reaction tube was charged with tert-butyl R-4-(7-(3,4-dichlorobenzoyl)-6-methyl-2-(methyl(1-methyl-1H-pyrazol-5-yl)amino)-4-oxo-5,6,7,8-tetrahydropyrido[3,4-d]pyrimidin-3(4H)-yl)benzoate I191 (147 mg, 0.24 mmol) in dry DCM (1 mL). TFA (0.5 mL, 6.53 mmol) was added and the mixture was stirred at RT for 2 h. The mixture was concentrated in vacuo. The residue was partitioned between water and Me-THF and neutralized with NaHCO₃. The organic layer was separated, dried (Na₂SO₄), filtered and concentrated in vacuo to become the title compound R-4-(7-(3,4-dichlorobenzoyl)-6-methyl-2-(methyl(1-methyl-1H-pyrazol-5-yl)amino)-4-oxo-5,6,7,8-tetrahydropyrido[3,4-d]pyrimidin-3(4H)-yl)benzoic acid I192 (124 mg, yield 93%).

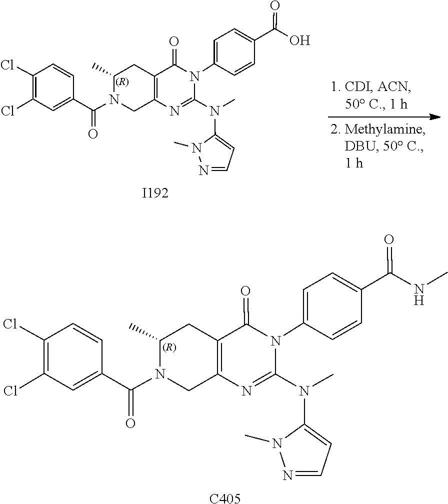

A reaction tube was charged with R-4-(7-(3,4-dichlorobenzoyl)-6-methyl-2-(methyl(1-methyl-1H-pyrazol-5-yl)amino)-4-oxo-5,6,7,8-tetrahydropyrido[3,4-d]pyrimidin-3(4H)-yl)benzoic acid I192 (124 mg, 0.22 mmol) in dry MeCN (3.8 mL). CDI (53 mg, 0.33 mmol) was added and the mixture was stirred at 50° C. for 1 h. The mixture was cooled to RT and methylamine (8.1 mg, 0.26 mmol) and DBU (0.0653 mL, 0.44 mmol) was added. The mixture was heated at 50° C. for 1 h. The mixture was cooled and concentrated in vacuo. The residue was purified by column chromatography (DCM/DCM-MeOH/NH₃(9-1) from 100/0 to 0/100). The product fractions were collected and concentrated in vacuo. The product was triturated in CH₃CN, filtered off and dried under vacuum to become the title compound C405 (40 mg, yield 32%) as a white solid.

### Procedure BE

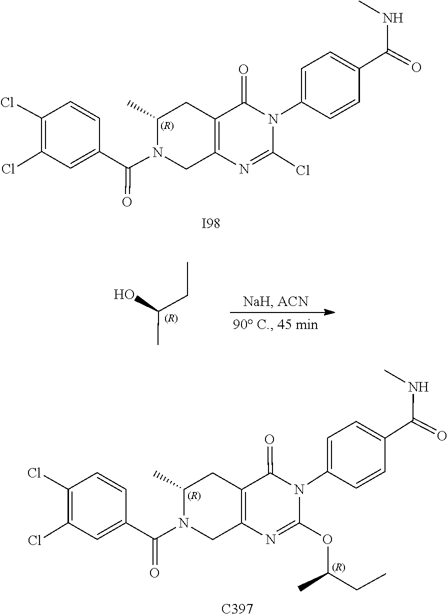

R-4-(2-chloro-7-(3,4-dichlorobenzoyl)-6-methyl-4-oxo-5,6,7,8-tetrahydropyrido[3,4-d]pyrimidin-3(4H)-yl)-N-methylbenzamide 198 (300 mg, 0.59 mmol) was dissolved in ACN (3 mL) and R-butan-2-ol (3 mL, 32.38 mmol). Sodium hydride (60% in mineral oil) (0.036 mg, 0.89 mmol) was added and the reaction vessel was sealed. The reaction mixture was heated at 90° C. for 45 min. The solvent was removed to afford a black oil. The crude was purified by flash chromatography (DCM/MeOH from 98/2 to 95/5) to afford crude product as a yellow oil. The oil was purified by flash chromatography over C18 silica gel (water/ACN from 80/20 to 40/60, 25 min) to afford the title compound 4-(R-2-(R-sec-butoxy)-7-(3,4-dichlorobenzoyl)-6-methyl-4-oxo-5,6,7,8-tetrahydropyrido[3,4-d]pyrimidin-3(4H)-yl)-N-methylbenzamide C397 (100 mg, yield 31%) as a white solid.

The compounds listed below were synthetized in an analogous manner, starting from intermediate I98 or I132 and appropriate alcohol.

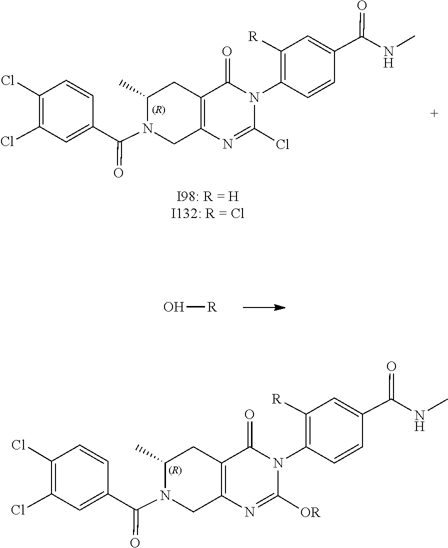

| Cmpd | |
|---|---|
| no | Structure |
| C397 | 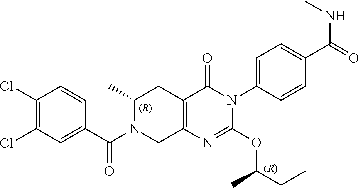 |
| C398 | 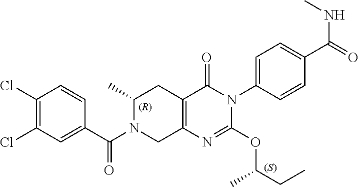 |
| C446 | 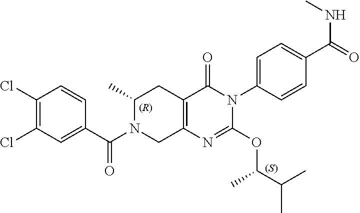 |
| C458 | 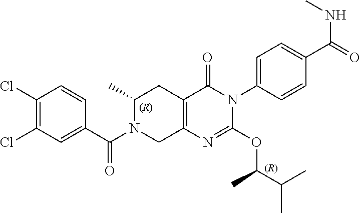 |
| C403 | 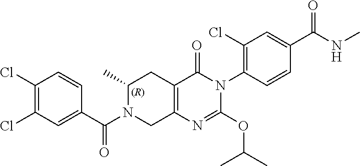 |

### SFC Separation

The compounds below have been purified by preparative SFC following one of the methods described in the general information “Separation of rotamers, diastereoisomers and enantiomers”

| Final compound | | Origin |
|---|---|---|
| 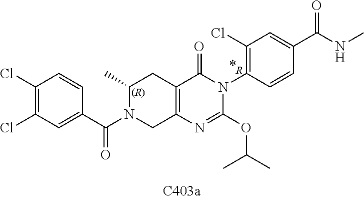 | 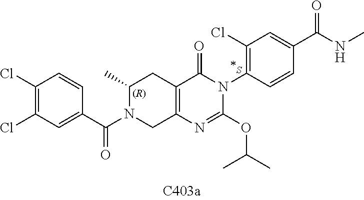 | C403 |

### Procedure BF

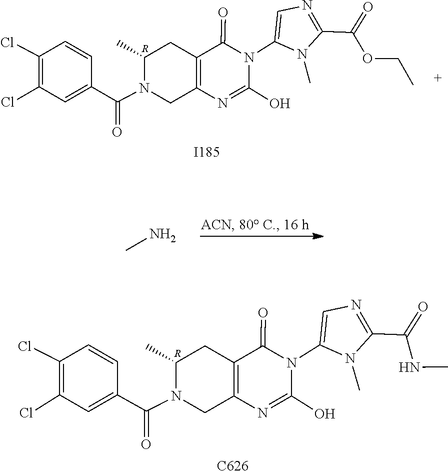

To the solution of ethyl R-5-(7-(3,4-dichlorobenzoyl)-2-hydroxy-6-methyl-4-oxo-5,6,7,8-tetrahydropyrido[3,4-d]pyrimidin-3(4H)-yl)-1-methyl-1H-imidazole-2-carboxylate I185 (17 mg, 0.034 mmol) in AGN (0.2 ml), methylamine (10 mg, 0.37 mmol) was added and stirred at 80° C. for 16 h. The solvent was removed under reduced pressure. A purification was performed via Prep HPLC (Stationary phase: RP Xbridge Prep C18 OBD-5 μm, 30×250 mm, Mobile phase: 0.25% NH₄HCO₃ solution in water, CH₃CN) yielding the title compound R-5-(7-(3,4-dichlorobenzoyl)-2-hydroxy-6-methyl-4-oxo-5,6,7,8-tetrahydropyrido[3,4-d]pyrimidin-3(4H)-yl)-N,1-dimethyl-1H-imidazole-2-carboxamide C626 (4.3 mg, yield 26%).

### Procedure BG

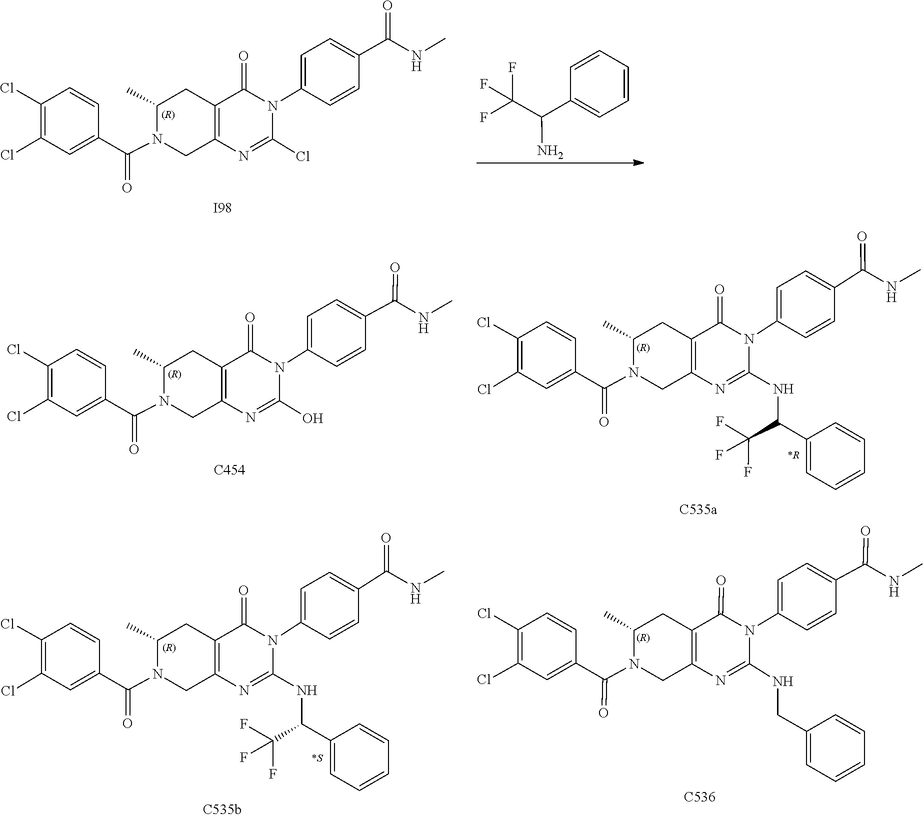

I98 (300 mg, 0.498 mmol) and 2,2,2-trifluoro-1-phenylethylamine (1 g, 5.71 mmol) were stirred at 100° C. for 2 days. LCMS showed no 198 left and a mixture of C454/C535 and C536. Water and EtOAc were added and the organic layer was separated. The aqueous layer was extracted with EtOAc and the combined organic layers were dried over Na₂SO₄, filtered and concentrated. The residue was This crude was then purified by column chromatography (DCM/MeOH 100/0 to 95/5) to afford C535 (62 mg, yield 19%), C536 (39 mg, yield 12%) and C454 (132 mg, yield 54%). C535 was purified by via Prep SFC (Stationary phase: Chiralpak Daicel ID 20×250 mm, Mobile phase: CO₂, i-PrOH+0.4 i-PrNH₂) to afford C535a (22 mg, yield 7%) and C535b (26 mg, yield 8%).

### Procedure BH

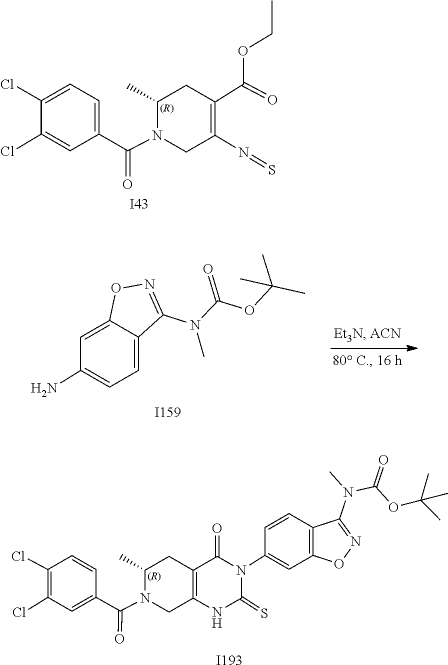

Ethyl (2R)-1-(3,4-dichlorobenzoyl)-5-isothiocyanato-2-methyl-3,6-dihydro-2H-pyridine-4-carboxylate I43 (1.792 g, 4.489 mmol), tert-butyl (6-aminobenzo[d]-isoxazol-3-yl)(methyl)carbamate I159 (1.3 g, 4.94 mmol), Et₃N (0.94 mL) in dry CH₃CN (25.5 mL).The mixture was stirred at 80° C. for 16 h. The mixture was cooled and the mixture was concentrated in vacuo. The residue was purified by column chromatography (heptane/EtOAc from 100/0 to 0/100). The product fractions were collected and concentrated in vacuo to become the title compound tert-butyl (R)-(6-(7-(3,4-dichlorobenzoyl)-6-methyl-4-oxo-2-thioxo-1,4,5,6,7,8-hexahydropyrido[3,4-d]pyrimidin-3(2H)-yl)benzo[d]isoxazol-3-yl)(methyl)carbamate I193 (1.8 g, yield 65%) as a yellow solid.

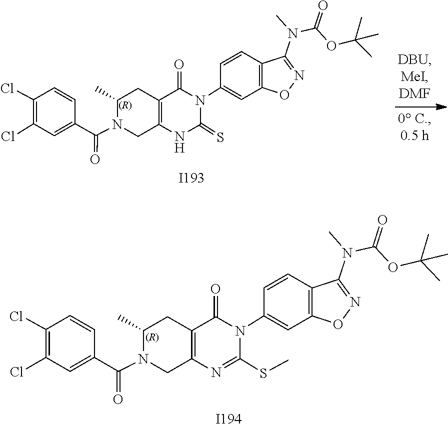

To a solution of tert-butyl (R)-(6-(7-(3,4-dichlorobenzoyl)-6-methyl-4-oxo-2-thioxo-1,4,5,6,7,8-hexahydropyrido[3,4-d]pyrimidin-3(2H)-yl)benzo[d]isoxazol-3-yl)-(methyl)carbamate I193 (1.8 g, 2.92 mmol) in DMF (12 mL) were added DBU (0.52 mL, 3.504 mmol) and Mel (0.2 mL, 3.21 mmol) at 0° C. The reaction mixture was stirred at 0° C. for 0.5 hour, and the reaction was quenched with a saturated aqueous sodium hydrogen carbonate solution. The mixture was extracted with Me-THF, and the organic layer was dried over MgSO₄, filtered, and concentrated under reduced pressure. The residue was purified by column chromatography (heptane/EtOAc from 100/0 to 0/100) to afford the title compound tert-butyl (R)-(6-(7-(3,4-dichlorobenzoyl)-6-methyl-2-(methylthio)-4-oxo-5,6,7,8-tetrahydropyrido[3,4-d]pyrimidin-3(4H)-yl)benzo[d]isoxazol-3-yl)(methyl)carbamate I194 (1.6 g, yield 87%) as a pale yellow foam.

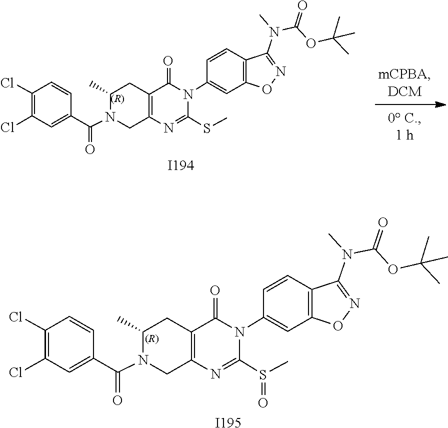

A solution of tert-butyl (R)-(6-(7-(3,4-dichlorobenzoyl)-6-methyl-2-(methylthio)-4-oxo-5,6,7,8-tetrahydropyrido[3,4-d]pyrimidin-3(4H)-yl)benzo[d]isoxazol-3-yl)(methyl)-carbamate I194 (500 mg, 0.79 mmol) in dry DCM (11 mL) was cooled to 0° C. and mCPBA (213 mg, 0.95 mmol) was added. The resulting reaction was allowed to stir for 1 h at 0° C. K₂CO₃ (497.5 mg, 3.6 mmol) was added and the mixture was stirred at RT for 30 min. The mixture was filtered and concentrated in vacuo to become the title compound tert-butyl (6-((6R)-7-(3,4-dichlorobenzoyl)-6-methyl-2-(methylsulfinyl)-4-oxo-5,6,7,8-tetrahydropyrido[3,4-d]pyrimidin-3(4H)-yl)benzo[d]isoxazol-3-yl)-(methyl)carbamate I195 (502 mg, yield 98%) as a yellow foam. The product was used as such in the next step.

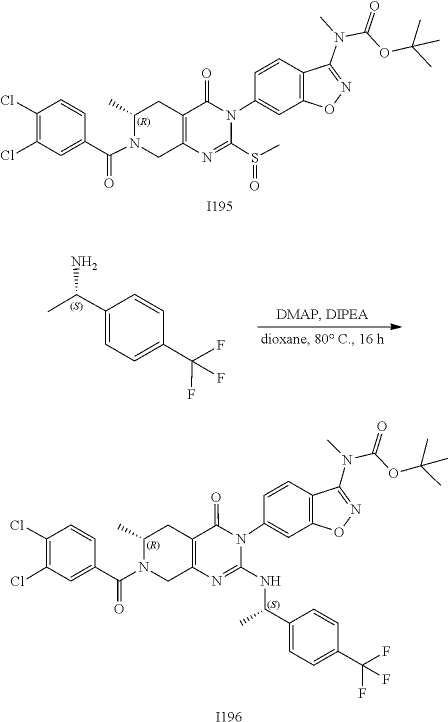

A reaction vial was charged with tert-butyl (6-((6R)-7-(3,4-dichlorobenzoyl)-6-methyl-2-(methylsulfinyl)-4-oxo-5,6,7,8-tetrahydropyrido[3,4-d]pyrimidin-3(4H)-yl)benzo[d]-isoxazol-3-yl)(methyl)carbamate I195 (502 mg, 0.78 mmol), (S)-1-[4-(trifluoromethyl)-phenyl]ethylamine (147 μL, 0.932 mmol), DIPEA (0.17 mL, 1.01 mmol) and DMAP (9.5 mg, 0.078 mmol) in dry 1,4-dioxane (7.3 mL). The mixture was stirred at 80° C. for 16 h. The mixture was cooled to RT and concentrated in vacuo. The residue was purified by column chromatography (heptane/EtOAc from 100/0 to 0/100) to afford the title compound tert-butyl (6-((R)-7-(3,4-dichlorobenzoyl)-6-methyl-4-oxo-2-(((S)-1-(4-(trifluoromethyl)phenyl)ethyl)amino)-5,6,7,8-tetrahydropyrido[3,4-d]pyrimidin-3(4H)-yl)benzo[d]isoxazol-3-yl)(methyl)carbamate I196 (63 mg, yield 11%) which was used as such in the next step.

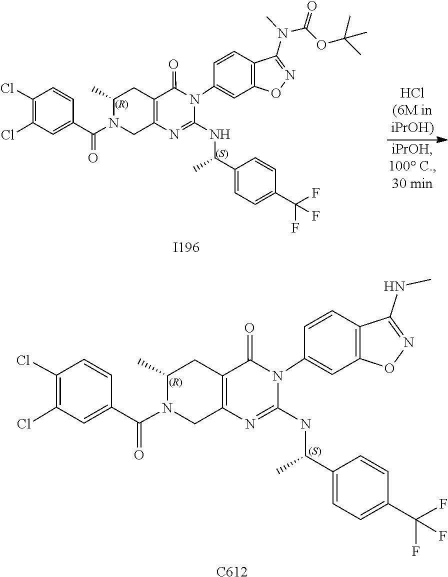

A reaction tube was charged with tert-butyl (6-((R)-7-(3,4-dichlorobenzoyl)-6-methyl-4-oxo-2-(((S)-1-(4-(trifluoromethyl)phenyl)ethyl)amino)-5,6,7,8-tetrahydropyrido-[3,4-d]pyrimidin-3(4H)-yl)benzo[d]isoxazol-3-yl)(methyl)carbamate (63 mg, 0.082 mmol) in i-PrOH (0.52 mL). HCl (0.14 mL, 6 M in i-PrOH, 0.82 mmol) was added and the mixture was heated at 100° C. for 30 min. The mixture was cooled and concentrated in vacuo. The residue was portioned between sat. NaHCO₃ solution and Me-THF. The organic layer was separated, dried over MgSO₄, filtered and concentrated in vacuo. A purification was performed via Prep HPLC (Stationary phase: RP XBridge Prep C18 OBD-10 μm, 30×150 mm, Mobile phase: 0.25% NH₄HCO₃ solution in water, CH₃CN). The product fractions were collected and concentrated in vacuo. The residue was dissolved in MeOH and concentrated in vacuo. The product was triturated in DIPE, filtered off and dried under vacuum to afford the title compound (R)-7-(3,4-dichlorobenzoyl)-6-methyl-3-(3-(methylamino)-benzo[d]isoxazol-6-yl)-2-(((S)-1-(4-(trifluoromethyl)phenyl)ethyl)amino)-5,6,7,8-tetrahydropyrido[3,4-d]pyrimidin-4(3H)-one C612 (14 mg, yield 26%) as a white solid.

The compounds listed below were synthetized in an analogous manner.

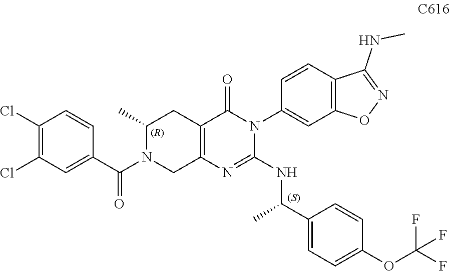

using I196 and (S)-1-[4-(trifluoromethoxy)phenyl]ethylamine.

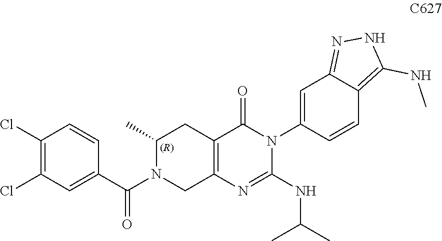

starting from Ethyl (2R)-1-(3,4-dichloro-benzoyl)-5-isothiocyanato-2-methyl-3,6-dihydro-2H-pyridine-4-carboxylate I43 and I154. C627 was obtained after silyl deprotection using HCl 1M in i-PrOH 10 eq. 1h at 90° C.

### Procedure BI

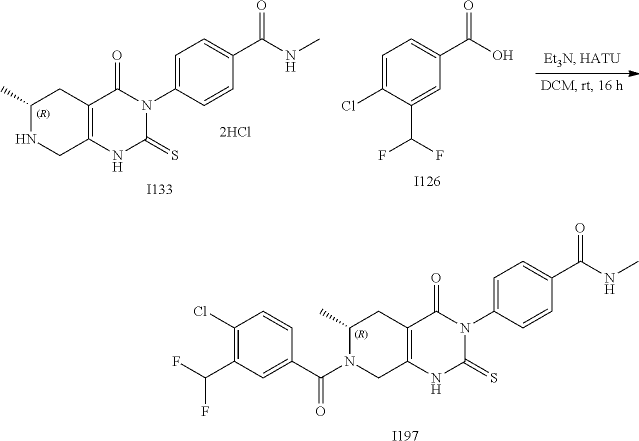

(R)-N-methyl-4-(6-methyl-4-oxo-2-thioxo-1,4,5,6,7,8-hexahydropyrido[3,4-d]-pyrimidin-3(2H)-yl)benzamide.2hydrochloride I133 (315 mg, 0.71 mmol) was added to a solution of 4-chloro-3-(difluoromethyl)benzoic acid I126 (135 mg, 0.65 mmol), dry triethylamine (0.5 mL, 3.70 mmol) and HBTU (397 mg, 1.05 mmol) in dry DCM (8 mL). The mixture was stirred at RT for 16 h. The solvent was removed and the crude was purified by column chromatography (heptane/EtOAc from 100/0 to 0/100) to afford the title compound (R)-4-(7-(4-chloro-3-(difluoromethyl)benzoyl)-6-methyl-4-oxo-2-thioxo-1,4,5,6,7,8-hexahydropyrido[3,4-d]pyrimidin-3(2H)-yl)-N-methylbenzamide I197 (311 mg, yield 74%) as a white powder.

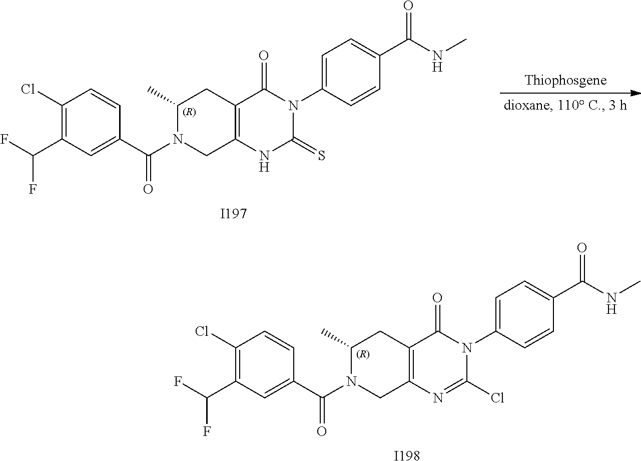

(R)-4-(7-(4-chloro-3-(difluoromethyl)benzoyl)-6-methyl-4-oxo-2-thioxo-1,4,5,6,7,8-hexahydropyrido[3,4-d]pyrimidin-3(2H)-yl)-N-methylbenzamide I197 (311 mg, 0.49 mmol) was suspended in dry 1,4-dioxane (5 mL) and then thiophosgene (0.15 mL, 1.96 mmol) was added. The reaction vessel was closed in a N₂-atmosphere. The bright orange mixture was stirred 3 h at 110° C. The solvent was removed under reduced pressure at 40° C. and the residue was purified by column chromatography (heptane/EtOAc from 100/0 to 0/100) afford the title compound (R)-4-(2-chloro-7-(4-chloro-3-(difluoromethyl)benzoyl)-6-methyl-4-oxo-5,6,7,8-tetrahydropyrido[3,4-d]pyrimidin-3(4H)-yl)-N-methylbenzamide I198 (169 mg, yield 41%).

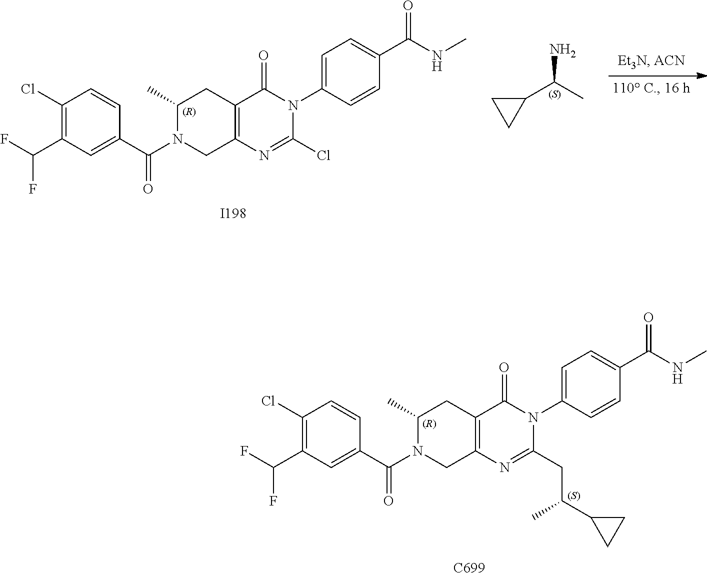

To a solution (R)-4-(2-chloro-7-(4-chloro-3-(difluoromethyl)benzoyl)-6-methyl-4-oxo-5,6,7,8-tetrahydropyrido[3,4-d]pyrimidin-3(4H)-yl)-N-methylbenzamide I198 (169 mg, 0.20 mmol) and dry Et₃N (0.2 mL, 1.44 mmol) in dry ACN (3 mL) was added (S)-1-cyclopropylethylamine (64 mg, 0.75 mmol) and the microwave tube was sealed under a N₂-atmosphere. The reaction mixture was heated at 110° C. for 16 h. The solvent was removed and the crude was purified via Prep HPLC (Stationary phase: RP XBridge Prep C18 OBD-10 μm, 50×150 mm, Mobile phase: 0.25% NH₄HCO₃ solution in water, CH₃CN). The product was recrystallized with a mixture of EtOH (˜2 mL) and water (˜4 mL). The solid was collected by filtration and the product was further purified via Prep SFC (Stationary phase: Chiralpak Diacel AD 20×250 mm, Mobile phase: CO₂, EtOH+0.4 i-PrNH₂) to afford the title compound 4-((R)-7-(4-chloro-3-(difluoromethyl)benzoyl)-2-(((S)-1-cyclopropylethyl)amino)-6-methyl-4-oxo-5,6,7,8-tetrahydropyrido[3,4-d]pyrimidin-3(4H)-yl)-N-methylbenzamide C699 (45 mg, yield 39%) as a white powder.

### Procedure BJ

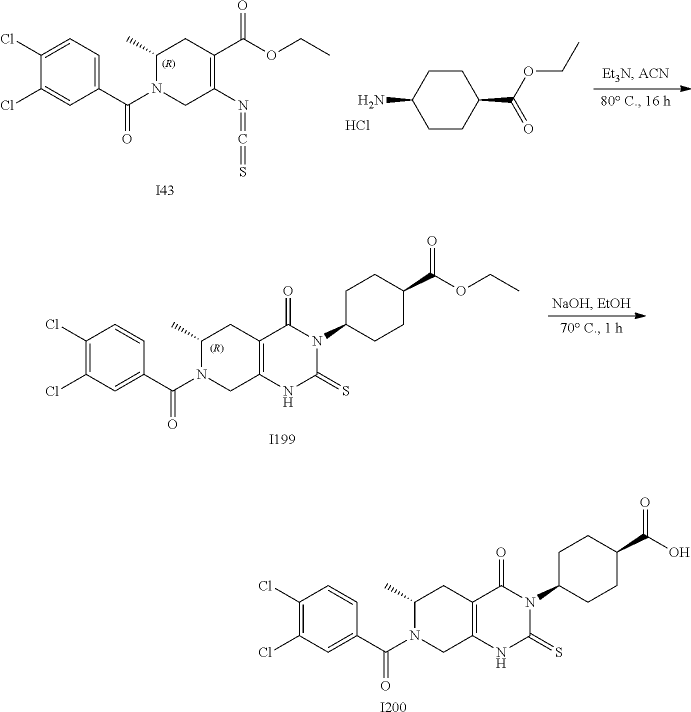

A reaction tube was charged with cis-ethyl 4-aminocyclohexanecarboxylate hydrochloride (624 mg, 3.01 mmol) in dry CH₃CN (14.2 mL). Et₃N (0.87 mL, 6.26 mmol) was added and the mixture was stirred for 5 min. at RT. Ethyl-(2R)-1-(3,4-dichlorobenzoyl)-5-isothiocyanato-2-methyl-3,6-dihydro-2H-pyridine-4-carboxylate I43 (1 g, 2.50 mmol) was added and the mixture was stirred at RT. for 10 min. The reaction mixture was further heated at 80° C. for 16 h. The mixture was cooled and concentrated in vacuo. NaOH (1 g, 25.00 mmol) was dissolved in EtOH (10 mL) and this solution was added to the residue under N₂ atmosphere. The mixture was heated at 70° C. for 1 h. The mixture was cooled and acidified with 1N HCl solution. The mixture was extracted with Me-THF and the organic layer was dried over MgSO₄, filtered and concentrated in vacuo. The product was triturated in CH₃CN, filtered off and washed with DIPE to become the title compound (1 S,4s)-4-((R)-7-(3,4-dichlorobenzoyl)-6-methyl-4-oxo-2-thioxo-1,4,5,6,7,8-hexahydropyrido[3,4-d]pyrimidin-3(2H)-yl)cyclohexane-1-carboxylic acid I200 (785 mg, yield 63%) as a white solid.

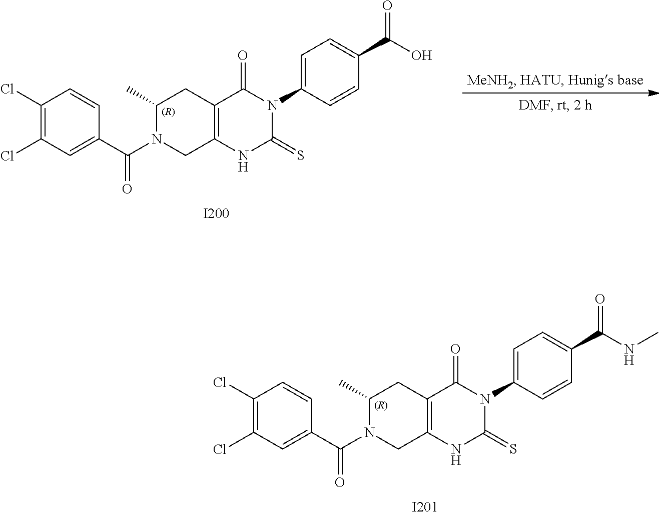

A reaction tube was charged with (1 S,4s)-4-((R)-7-(3,4-dichlorobenzoyl)-6-methyl-4-oxo-2-thioxo-1,4,5,6,7,8-hexahydropyrido[3,4-d]pyrimidin-3(2H)-yl)cyclohexane-1-carboxylic acid I200 (785 mg, 1.58 mmol) in DMF (10.2 mL). Hunig's base (0.354 mL, 2.06 mmol) and HATU (721.534 mg, 1.898 mmol) was added and the mixture was stirred at r.t for 1 h. Methylamine (58.935 mg, 1.898 mmol) was added and the mixture was stirred at RT for 1 h. The mixture was concentrated in vacuo. The residue was purified by column chromatography (heptane/EtOAC from 100/0 to 0/100). The obtained product was triturated in DIPE, filtered and dried under vacuum to become the title compound (1 S,4s)-4-((R)-7-(3,4-dichlorobenzoyl)-6-methyl-4-oxo-2-thioxo-1,4,5,6,7,8-hexahydropyrido[3,4-d]pyrimidin-3(2H)-yl)-N-methylcyclohexane-1-carboxamide I201 (700 mg, yield 87%) as a pale yellow solid.

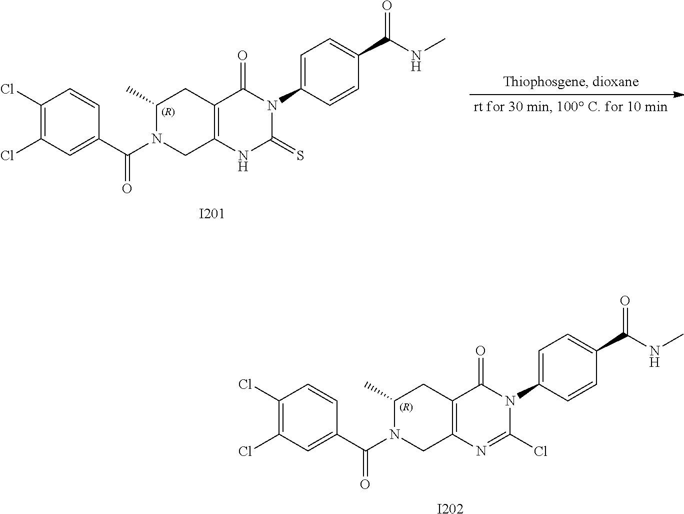

A reaction vial was charged with (1 S,4s)-4-((R)-7-(3,4-dichlorobenzoyl)-6-methyl-4-oxo-2-thioxo-1,4,5,6,7,8-hexahydropyrido[3,4-d]pyrimidin-3(2H)-yl)-N-methyl-cyclohexane-1-carboxamide I201 (300 mg, 0.59 mmol) in dry 1,4-dioxane (3 mL). Thiophosgene (51 μL, 0.65 mmol) was added and the mixture was stirred at RT for 30 min. The mixture was then heated at 100° C. for 10 min. The mixture was concentrated in vacuo. The residue was purified by column chromatography (heptane/EtOAc-EtOH(3:1) from 100/0 to 0/100) to the title compound (1S,4s)-4-((R)-2-chloro-7-(3,4-dichlorobenzoyl)-6-methyl-4-oxo-5,6,7,8-tetrahydropyrido[3,4-d]pyrimidin-3(4H)-yl)-N-methylcyclohexane-1-carboxamide I202 (175 mg, yield 58%) as a yellow solid.

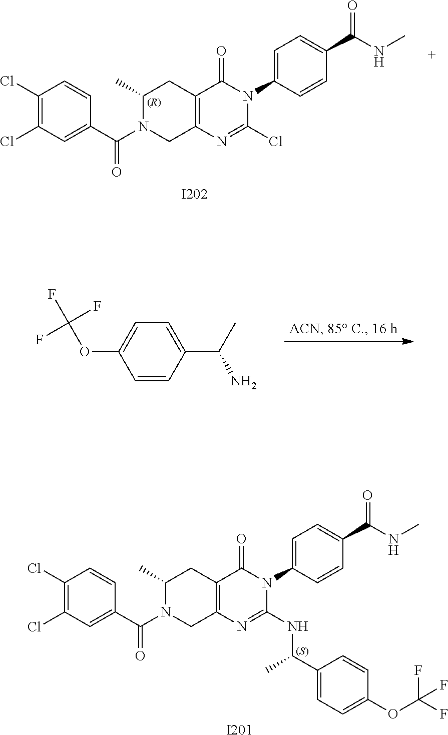

A reaction tube was charged with compound (1 S,4s)-4-((R)-2-chloro-7-(3,4-dichloro-benzoyl)-6-methyl-4-oxo-5,6,7,8-tetrahydropyrido[3,4-d]pyrimidin-3(4H)-yl)-N-methylcyclohexane-1-carboxamide I202 (175 mg, 0.342 mmol), (S)-1-(4-(trifluoro-methoxy)phenyl)ethanamine (175 mg, 0.86 mmol) in dry CH₃CN (1.9). The mixture was heated at 85° C. for 16 h. The mixture was cooled and concentrated in vacuo. The product was purified by column chromatography (heptane/EtOAc from 100/0 to 0/100). The obtained oil was crystallized from water-MeOH, filtered off and dried under vacuum to become the title compound (1R,4s)-4-((R)-7-(3,4-dichlorobenzoyl)-6-methyl-4-oxo-2-(((S)-1-(4-(trifluoromethoxy)phenyl)ethyl)amino)-5,6,7,8-tetrahydropyrido[3,4-d]pyrimidin-3(4H)-yl)-N-methylcyclohexane-1-carboxamide C636 (122 mg, yield 52%) as a white solid.

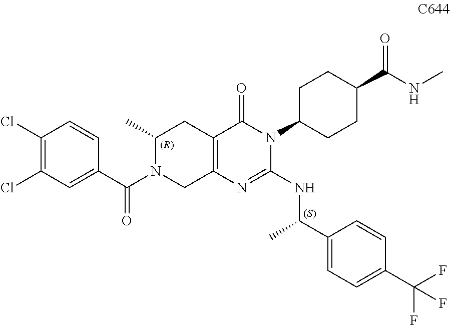

was synthetized in an analogous manner starting from I202 and (S)-1-[4-(trifluoromethyl)phenyl]ethylamine.

### Procedure BK

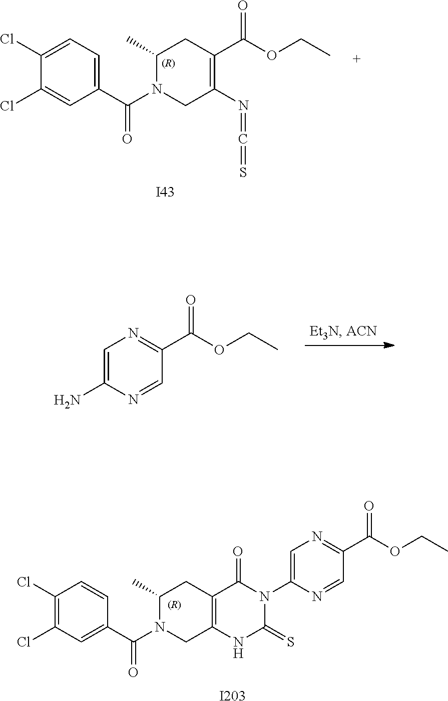

A μ-wave tube was charged with 143 (500 mg, 1.252 mmol), ethyl 5-aminopyrazine-2-carboxylate (272 mg, 1.63 mmol) and Et₃N (435 μL, 3.13 mmol) in dry CH₃CN (7 mL) and then sealed in a N₂-atm. The mixture was heated at 90° C. for 9 days. The solvents were removed under reduced pressure and the crude was purified by silica eluting (DCM/MeOH from 100/0 to 90/10) to afford I203 (265 mg, 70% pure, yield 28%) as a brown foam which was used as such in the next step.

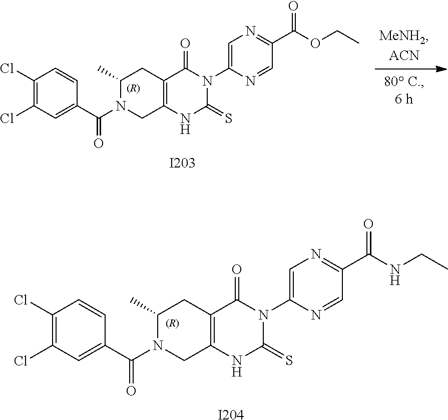

Ethyl (R)-5-(7-(3,4-dichlorobenzoyl)-6-methyl-4-oxo-2-thioxo-1,4,5,6,7,8-hexahydropyrido[3,4-d]pyrimidin-3(2H)-yl)pyrazine-2-carboxylate I203 (2.18 g, 3.26 mmol) was dissolved in dry ACN (29 mL)) and a few drops of pure methylamine was added and the vessel was sealed. The reaction mixture was heated at 80° C. for 5 h. The mixture was cooled to RT and the solvent was evaporated. The crude was purified via silica column chromatography (DCM/MeOH from 100/0 to 90/10). The obtained foam was triturated in DIPE and the formed solid was filtered to yield the title compound (R)-5-(7-(3,4-dichlorobenzoyl)-6-methyl-4-oxo-2-thioxo-1,4,5,6,7,8-hexahydropyrido[3,4-d]pyrimidin-3(2H)-yl)-N-methylpyrazine-2-carboxamide I204 (0.52 g, yield 32%) as a beige powder.

The compounds listed below were obtained was obtained by following steps similar to the synthesis of C636.

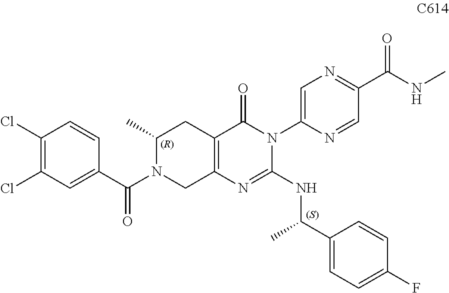

using (S)-1-(4-fluorophenyl)ethylamine.

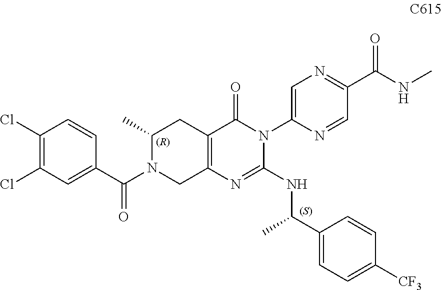

starting from (S)-1-[4-(trifluoromethyl)phenyl]ethylamine

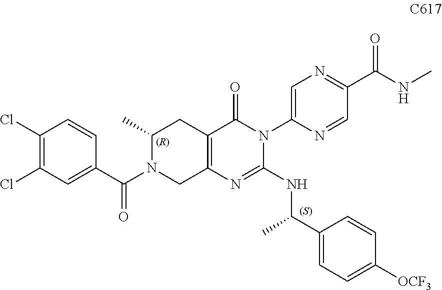

starting from (S)-1-(4-(trifluoromethoxy)phenyl)ethanamine

### Procedure BM

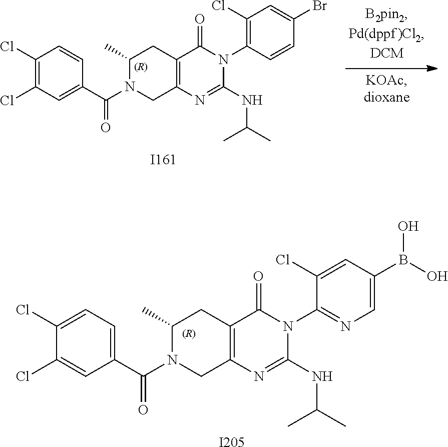

I161 (1.5 g, 2.57 mmol), bis(pinacolato)diboron (1.3 mg, 5.13 mmol), potassium acetate (528 mg, 5.39 mmol) and 1,1′-bis(diphenylphosphino)ferrocene-palladium(II)dichloride dichloromethane complex (212 mg, 0.26 mmol) were dissolved in 1,4-dioxane (10 mL). The reaction mixture was heated 48 hours in a heating block at 85° C. The reaction mixture was cooled down to room temperature and the volatiles were removed under reduced pressure. The residue was purified on silica column chromatography (heptane/EtOAc from 100/0 to 0/100) to afford I205 (633 mg, yield 45%).

### Synthesis of Compound C371, C392, C455

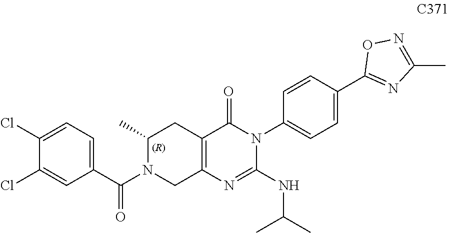

was obtained following procedure AH (condition B) starting from I74 and 5-bromo-3-methyl-1,2,4-oxadiazole,

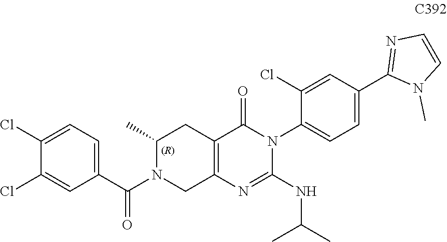

was obtained following procedure AH (condition B) starting from I205 and 2-bromo-1-methyl-1H-imidazole.

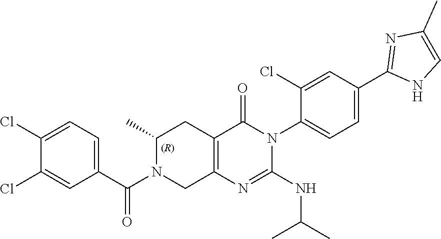

C455 was obtained following procedure AH (condition B) starting from I205 and I160 followed by the amino-Boc deprotection described below

Tert-butyl (R)-2-(3-chloro-4-(7-(3,4-dichlorobenzoyl)-2-(isopropylamino)-6-methyl-4-oxo-5,6,7,8-tetrahydropyrido[3,4-d]pyrimidin-3(4H)-yl)phenyl)-4-methyl-1H-imidazole-1-carboxylate (57 mg, 0.083 mmol) was dissolved in DCM (5 mL) and TFA (94 mg, 0.83 mmol) was added. The resulting mixture was stirred at RT for 16 h. The solvents was removed and the residue was purified on silica column chromatography (DCM/MeOH 100/0 to 90/10) to afford C455 (40 mg, 82% yield) as a white solid.

### Procedure BN

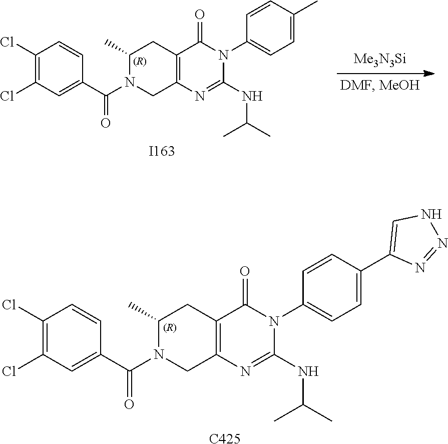

A microwave vial was charged with I163 (500 mg, 1.01 mmol), copper iodide (9.61 mg, 0.05 mmol), DMF (1.8 mL) and MeOH (0.2 mL). Azidotrimethylsilane (211 μL, 1.51 mmol) was added and the mixture was heated 16 h at 100° C. The volatiles were removed under reduced pressure and the residue was purified on silica column chromatography (heptane/EtOAc 100/0 to 0/100). The resulting product was triturated in CH₃CN, filtered off and dried under vacuum to afford C425 (150 mg, yield 28%) as a solid.

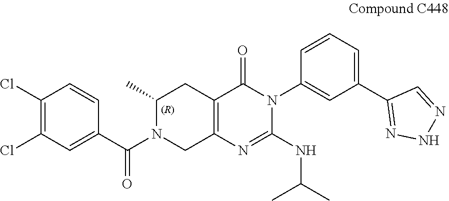

was obtained in analogous manner starting from I164,

### Procedure BO

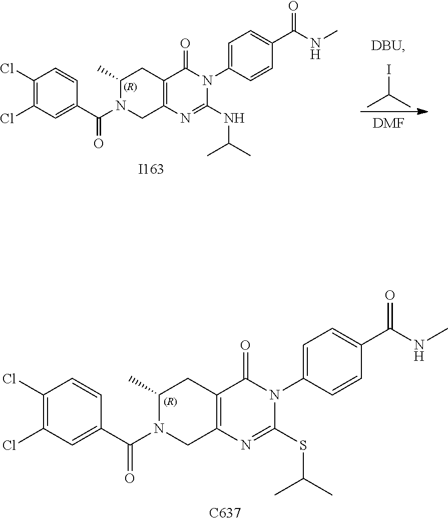

To a solution of I87 (199 mg, 0.38 mmol) in dry DMF (3 mL) were added 1,8-diazabicyclo[5.4.0]undec-7-ene (85 μL, 0.57 mmol) and 2-iodopropane (80 μL, 0.80 mmol). The reaction mixture was stirred 4 h at 0° C. followed by 30 min at RT. The mixture was concentrated under reduced pressure at 40° C. The residue was purified by column chromatography (heptane/ethyl acetate from 100/0 to 0/100). The obtained product was recrystallized with ACN (˜6 mL) to afford C637 (90 mg, yield 44%) as a white powder.

### Procedure BP

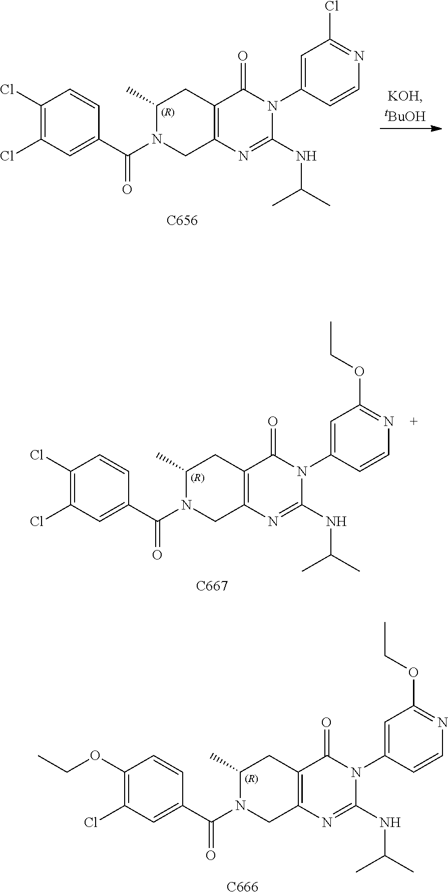

KOH was added to a solution of C656 (80 mg, 0.158 mmol) in t-BuOH (6 mL). The reaction mixture was stirred 6 h at reflux. LCMS showed no conversion. KOH (180 mg, 3.208 mmol) was added and the mixture was stirred overnight at reflux. LCMS showed no conversion.

The reaction was transferred in a microwave tube and the reaction was stirred in the microwave 30 min. at 150° C. [inadvertently, EtOH was used during the transfer in the microwave tube]. LCMS showed a conversion in C666 and C667. The reaction was cooled down and purified by prep HPLC (Stationary phase: RP XBridge Prep C18 OBD-10 μm, 50×150 mm, Mobile phase: 0.25% NH₄HCO₃ solution in water, CH₃CN) to afford C666 (6 mg, yield 7%) as a light-yellow foam and C667 (18 mg, yield 22%) as a white powder.

The following compounds were also made according to the procedures described herein:

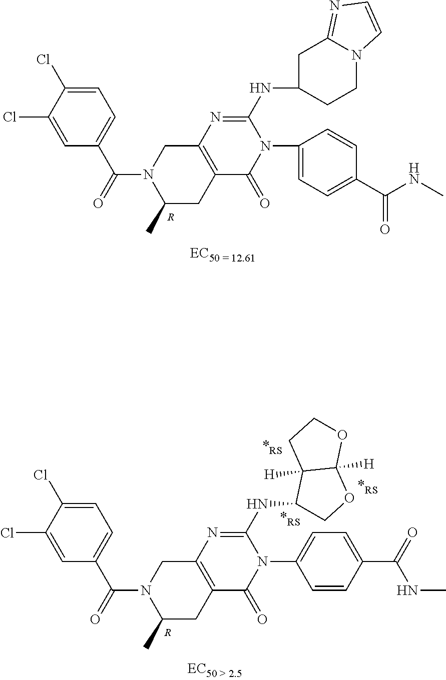

Compounds I89, I118-I120, I166, I190 and I191 are useful intermediates of synthesis and can also be considered final compounds.

X-Ray Crystallography Absolute configuration of C157 has been confirmed to be R by use of X-Ray crystallography. All intermediates were assigned accordingly.

The single crystal was obtained from Cooling in DMF followed by prolonged incubation at 5° C.

| | |
|---|---|
| Crystal System | Monoclinic |
| Space group | C2 |
| Unit cell dimensions | |
| a = 50.1456(14) Å | α = 90° |
| b = 5.8671(2) Å | β = 90.869(2)° |
| c = 10.2653(3) Å | γ = 90° |
| Volume | 3019.80(16) Å³ |
| Rfac = 6.58% | |

Large amount of disorder around the methylbenzamide and propenamine side groups of the API, however area around stereocenter is ordered and can confirm absolute stereochemistry.

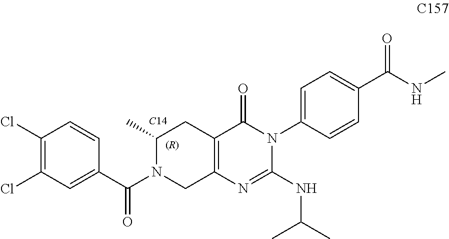

For the structure as presented, with the stereocenter in the R configuration at C14. The Flack parameter=−0.006(7), Acta Cryst. B69, 2013, 249-259.

Determination of the absolute structure using Bayesian statistics on Bijvoet differences, reveals that the probability of the absolute structure as presented being correct is 1.000, while the probabilities of the absolute structure being a racemic twin or false are both 0.000. The Flack equivalent and its uncertainty are calculated through this program to be=−0.010(6). The calculation was based on 2467 Bijvoet pairs with a coverage of 96%.

Hooft et al., J. Appl. Cryst., 2008, 41, 96-103.

### 5. Characterization of Compounds

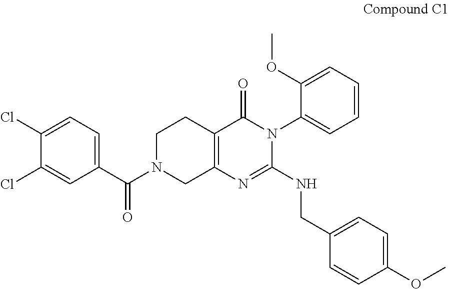 [SMILES: COc1ccc(CNc2nc3c(c(=O)n2-c2ccccc2OC)CCN(C(=O)c2ccc(Cl)c(Cl)c2)C3)cc1]

¹H NMR (400 MHz, DMSO-d₆, 80° C.) δ ppm 2.39 (t, J=5.8 Hz, 2H), 3.55-3.67 (m, 2H), 3.71 (s, 3H), 3.73 (s, 3H), 4.20-4.32 (m, 3H), 4.39 (dd, J=14.7, 6.1 Hz, 1H), 6.14 (br t, J=4.7 Hz, 1H), 6.81 (d, J=8.8 Hz, 2H), 7.05-7.18 (m, 4H), 7.21 (dd, J=8.4 Hz, 1H), 7.43 (dd, J=8.3, 1.9 Hz, 1H), 7.47 (ddd, J=8.3, 7.4, 1.9 Hz, 1H), 7.67-7.70 (m, 2H); LCMS (method B): Rt 2.08 min

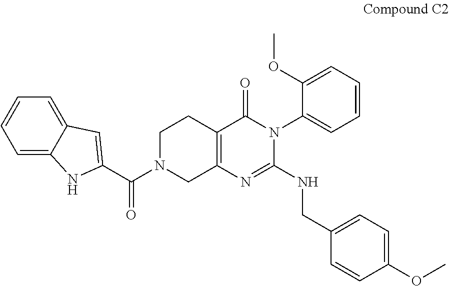 [SMILES: COc1ccc(CNc2nc3c(c(=O)n2-c2ccccc2OC)CCN(C(=O)c2cc4ccccc4[nH]2)C3)cc1]

¹H NMR (400 MHz, DMSO-d₆, 125° C.) δ ppm 2.49-2.52 (m, 2H), 3.70 (s, 3H), 3.72 (s, 3H), 3.89-3.95 (m, 2H), 4.27-4.34 (m, 1H), 4.38-4.47 (m, 1H), 4.51 (s, 2H), 5.81 (br s, 1H), 6.77-6.84 (m, 3H), 7.01-7.22 (m, 7H), 7.42-7.48 (m, 2H), 7.61 (d, J=7.9 Hz, 1H), 11.12 (br s, 1H); LCMS (method A): Rt 1.99 min

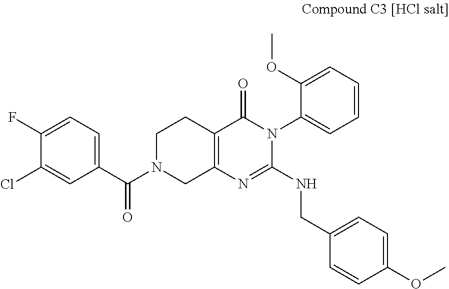 [SMILES: COc1ccc(CNc2nc3c(c(=O)n2-c2ccccc2OC)CCN(C(=O)c2ccc(F)c(Cl)c2)C3)cc1]

¹H NMR (400 MHz, DMSO-d₆, 125° C.) δ ppm 2.41 (t, J=5.8 Hz, 2H), 3.62 (t, J=5.8 Hz, 2H), 3.71 (s, 3H), 3.72 (s, 3H), 4.24-4.33 (m, 3H), 4.40 (dd, J=14.7, 5.6 Hz, 1H), 5.83 (br s, 1H), 6.78-6.83 (m, 2H), 7.05-7.16 (m, 4H), 7.20 (dd, J=8.4, 0.9 Hz, 1H), 7.38-7.48 (m, 3H), 7.62 (dd, J=7.2, 1.9 Hz, 1H); LCMS (method B): Rt 1.98 min

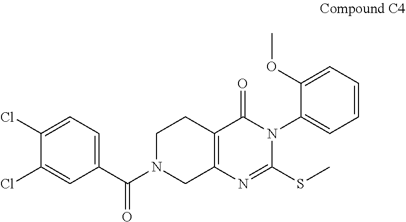 [SMILES: COc1ccccc1-n1c(SC)nc2c(c1=O)CCN(C(=O)c1ccc(Cl)c(Cl)c1)C2]

¹H NMR (400 MHz, DMSO-d₆) δ ppm 2.19-2.46 (m, 4H), 3.48-3.56 (m, 1H), 3.75 (s, 3H), 3.79-3.90 (m, 1H), 4.24-4.46 (m, 1H), 4.49-4.61 (m, 2H), 7.09 (t, J=6.9 Hz, 1H), 7.21-7.29 (m, 2H), 7.47-7.55 (m, 2H), 7.73-7.82 (m, 2H); LCMS (method C): Rt 1.13 min

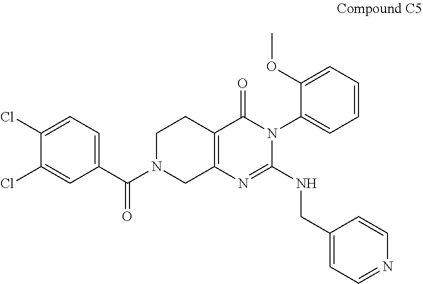 [SMILES: COc1ccccc1-n1c(NCc2ccncc2)nc2c(c1=O)CCN(C(=O)c1ccc(Cl)c(Cl)c1)C2]

¹H NMR (600 MHz, DMSO-d₆, 100° C.) δ ppm 2.39 (t, J=5.8 Hz, 2H), 3.60 (br s, 2H), 3.77 (s, 3H), 4.21 (brs, 2H), 4.34 (dd, J=16.1, 5.7 Hz, 1H), 4.49 (dd, J=16.1, 6.2 Hz, 1H), 6.38 (br t, J=5.9 Hz, 1H), 7.10 (td, J=7.6, 1.2 Hz, 1H), 7.18 (br d, J=5.3 Hz, 2H), 7.21 (dd, J=7.7, 1.7 Hz, 1H), 7.23 (dd, J=8.4, 1.0 Hz, 1H), 7.42 (dd, J=8.3, 2.0 Hz, 1H), 7.51 (ddd, J=8.3, 7.5, 1.7 Hz, 1H), 7.66-7.70 (m, 2H), 8.45 (d, J=5.8 Hz, 2H); LCMS (method C): Rt 0.93 min

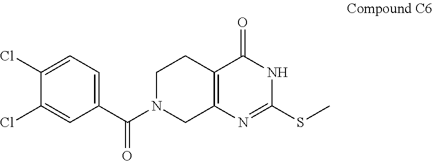 [SMILES: CSc1nc2c(c(=O)[nH]1)CCN(C(=O)c1ccc(Cl)c(Cl)c1)C2]

¹H NMR (400 MHz, DMSO-d₆, 60° C.) δ ppm 2.41-2.48 (m, 5H), 3.47-3.74 (m, 2H), 4.37 (br s, 2H), 7.44 (dd, J=8.1, 2.0 Hz, 1H), 7.69-7.73 (m, 2H), 12.52 (br s, 1H); LCMS (method B): Rt 1.63 min

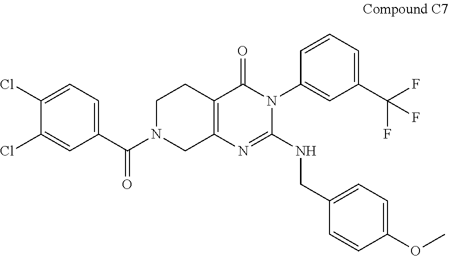 [SMILES: COc1ccc(CNc2nc3c(c(=O)n2-c2cccc(C(F)(F)F)c2)CCN(C(=O)c2ccc(Cl)c(Cl)c2)C3)cc1]

¹H NMR (400 MHz, DMSO-d₆, 100° C.) δ ppm 2.41 (t, J=5.8 Hz, 2H), 3.59-3.67 (m, 2H). 3.72 (s, 3H), 4.29 (s, 2H), 4.35 (s, 2H), 6.32 (br s, 1H), 6.78-6.83 (m, 2H), 7.15 (m, J=8.6 Hz, 2H), 7.41 (dd, J=8.1, 2.0 Hz, 1H), 7.57 (d, J=7.9 Hz, 1H), 7.62 (s, 1H), 7.66 (d, J=2.0 Hz, 1H), 7.68 (d, J=8.1 Hz, 1H), 7.74-7.80 (m, 1H), 7.80-7.86 (m, 1H); LCMS (method C): Rt 1.23 min

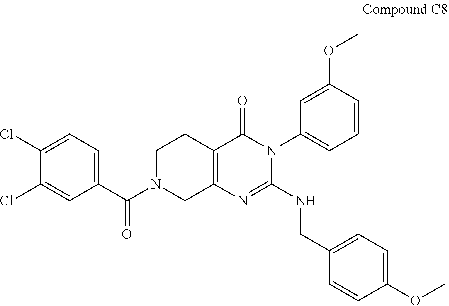 [SMILES: COc1ccc(CNc2nc3c(c(=O)n2-c2cccc(OC)c2)CCN(C(=O)c2ccc(Cl)c(Cl)c2)C3)cc1]

¹H NMR (400 MHz, DMSO-d₆, 100° C.) δ ppm 2.41 (t, J=5.8 Hz, 2H), 3.58-3.67 (m, 2H), 3.71 (s, 3H), 3.78 (s, 3H), 4.27 (s, 2H), 4.35 (br d, J=5.3 Hz, 2H), 5.98 (br s, 1H), 6.78-6.84 (m, 4H), 7.05 (ddd, J=8.5, 2.5, 0.9 Hz, 1H), 7.14 (d, J=8.8 Hz, 2H), 7.39-7.46 (m, 2H), 7.67 (d, J=11.5 Hz, 1H), 7.67 (s, 1H); LCMS (method C): Rt 1.16 min

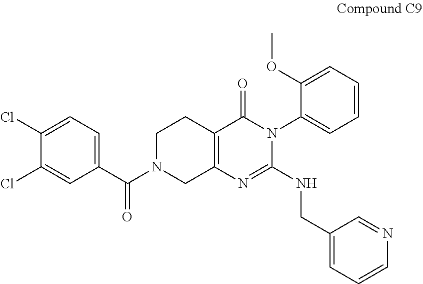 [SMILES: COc1ccccc1-n1c(NCc2cccnc2)nc2c(c1=O)CCN(C(=O)c1ccc(Cl)c(Cl)c1)C2]

¹H NMR (400 MHz, DMSO-d₆, 100° C.) δ ppm 2.39 (br s, 2H), 3.61 (br s, 2H), 3.72 (s, 3H), 4.26 (br s, 2H), 4.29-4.39 (m, 1H), 4.43-4.57 (m, 1H), 6.24-6.40 (m, 1H), 7.06-7.14 (m, 1H), 7.14-7.28 (m, 3H), 7.37-7.53 (m, 2H), 7.57-7.70 (m, 3H), 8.41 (br d, J=22.3 Hz, 2H); LCMS (method C): Rt 0.94 min

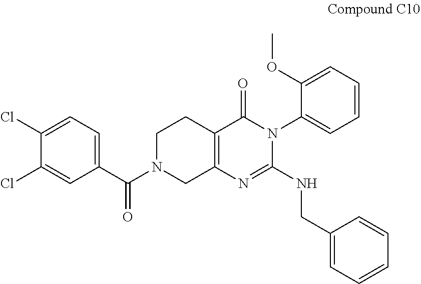 [SMILES: COc1ccccc1-n1c(NCc2ccccc2)nc2c(c1=O)CCN(C(=O)c1ccc(Cl)c(Cl)c1)C2]

¹H NMR (400 MHz, DMSO-d₆, 100° C.) δ ppm 2.40 (t, J=5.8 Hz, 2H), 3.55-3.65 (m, 2H), 3.74 (s, 3H), 4.25 (br s, 2H), 4.35 (d, J=15.3, 5.4 Hz, 1H), 4.48 (d, J=15.2, 5.9 Hz, 1H), 6.13 (br s, 1H), 7.05-7.12 (m, 1H), 7.14-7.28 (m, 7H), 7.41 (dd, J=8.1, 2.2 Hz, 1H), 7.45-7.51 (m, 1H), 7.65-7.68 (m, 2H); LCMS (method C): Rt 1.15 min

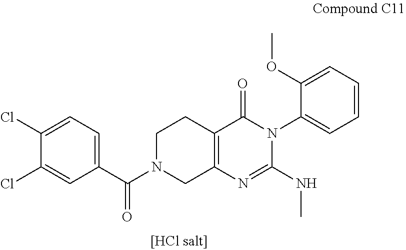 [SMILES: CNc1nc2c(c(=O)n1-c1ccccc1OC)CCN(C(=O)c1ccc(Cl)c(Cl)c1)C2]

¹H NMR (400 MHz, DMSO-d₆, 100° C.) δ ppm 2.40 (t, J=5.9 Hz, 2H), 2.71 (s, 3H), 3.56-3.69 (m, 2H), 3.74 (s, 3H), 4.24-4.40 (m, 2H), 5.67 (br s, 1H), 7.03-7.10 (m, 1H), 7.13 (dd, J=7.7, 2.0 Hz, 1H), 7.21 (dd, J=8.4, 1.1 Hz, 1H), 7.42-7.49 (m, 2H), 7.68 (d, J=1.1 Hz, 1H), 7.69 (d, J=5.1 Hz. 1H); LCMS (method C): Rt 0.95 min

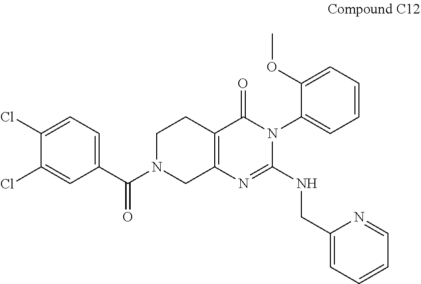 [SMILES: COc1ccccc1-n1c(NCc2ccccn2)nc2c(c1=O)CCN(C(=O)c1ccc(Cl)c(Cl)c1)C2]

¹H NMR (400 MHz, DMSO-d₆, 100° C.) δ ppm 2.41 (t, J=5.8 Hz, 2H), 3.56-3.67 (m, 2H), 3.76 (s, 3H), 4.24 (br s, 2H), 4.42-4.59 (m, 2H), 6.22 (br s, 1H), 7.08-7.14 (m, 1H), 7.18 (dd, J=7.3, 5.1 Hz, 1H), 7.21-7.27 (m, 3H), 7.41 (dd, J=8.1, 2.0 Hz, 1H), 7.47-7.53 (m, 1H), 7.63-7.73 (m, 3H), 8.37 (d, J=4.9 Hz, 1H); LCMS (method C): Rt 0.98 min

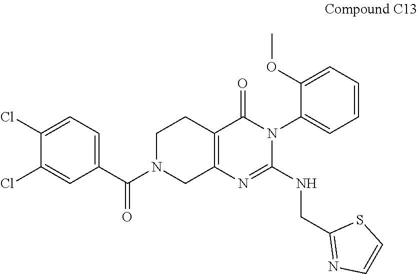 [SMILES: COc1ccccc1-n1c(NCc2nccs2)nc2c(c1=O)CCN(C(=O)c1ccc(Cl)c(Cl)c1)C2]

¹H NMR (400 MHz, DMSO-d₆, 100° C.) δ ppm 2.41 (br t, J=5.6 Hz, 2H), 3.50-3.67 (m, 2H), 3.74 (s, 3H), 4.28 (br s, 2H), 4.60-4.76 (m, 2H), 6.52 (br s, 1H), 7.05-7.25 (m, 3H), 7.40-7.51 (m, 3H), 7.62-7.69 (m, 3H); LCMS (method C): Rt 0.99 min

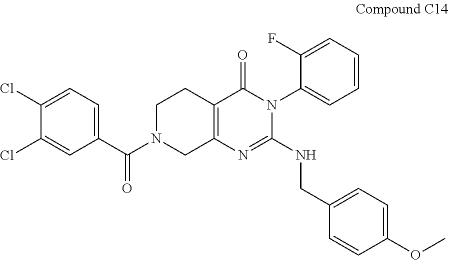 [SMILES: COc1ccc(CNc2nc3c(c(=O)n2-c2ccccc2F)CCN(C(=O)c2ccc(Cl)c(Cl)c2)C3)cc1]

¹H NMR (400 MHz, DMSO-d₆, 100° C.) δ ppm 2.41 (t, J=5.5 Hz, 2H), 3.53-3.67 (m, 2H), 3.72 (s, 3H), 4.29 (br s, 2H), 4.35 (s, 2H), 6.46 (br s, 1H), 6.81 (d, J=8.6 Hz, 2H), 7.15 (br d, J=8.6 Hz, 2H), 7.33-7.44 (m, 4H), 7.51-7.60 (m, 1H), 7.67 (d, J=5.6 Hz, 1H), 7.67 (s, 1H); LCMS (method A): Rt 2.17 min

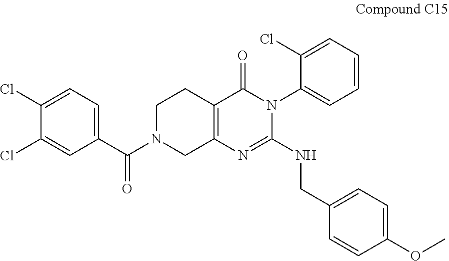 [SMILES: COc1ccc(CNc2nc3c(c(=O)n2-c2ccccc2Cl)CCN(C(=O)c2ccc(Cl)c(Cl)c2)C3)cc1]

¹H NMR (400 MHz, DMSO-d₆, 100° C.) δ ppm 2.42 (br t, J=5.7 Hz, 2H), 3.53-3.70 (m, 2H), 3.71 (s, 3H), 4.23-4.42 (m, 4H), 6.32 (br s, 1H), 6.78-6.83 (m, 2H), 7.15 (m, J=8.6 Hz, 2H), 7.36-7.45 (m, 2H), 7.47-7.56 (m, 2H), 7.62-7.71 (m, 3H); LCMS (method A): Rt 2.19 min

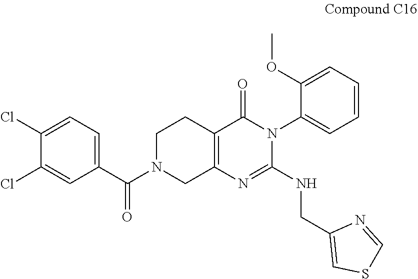 [SMILES: COc1ccccc1-n1c(NCc2cscn2)nc2c(c1=O)CCN(C(=O)c1ccc(Cl)c(Cl)c1)C2]

¹H NMR (400 MHz, DMSO-d₆, 100° C.) δ ppm 2.41 (t, J=5.8 Hz, 2H), 3.57-3.66 (m, 2H), 3.74 (s, 3H), 4.27 (s, 2H), 4.49-4.62 (m, 2H), 6.01-6.08 (m, 1H), 7.07-7.12 (m, 1H), 7.18-7.27 (m, 3H), 7.42 (dd, J=8.3, 1.9 Hz, 1H), 7.45-7.51 (m, 1H), 7.65-7.69 (m, 2H), 8.91 (s, 1H); LCMS (method C): Rt 0.96 min

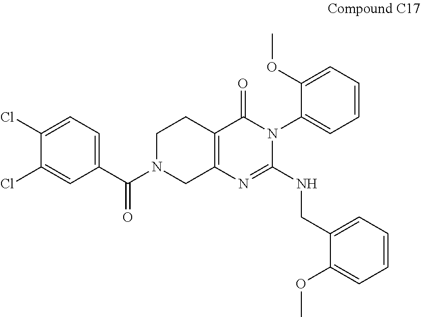 [SMILES: COc1ccccc1CNc1nc2c(c(=O)n1-c1ccccc1OC)CCN(C(=O)c1ccc(Cl)c(Cl)c1)C2]

¹H NMR (400 MHz, DMSO-d₆, 100° C.) δ ppm 2.40 (t, J=5.8 Hz, 2H), 3.58-3.64 (m, 2H), 3.68 (s, 3H), 3.73 (s, 3H), 4.26 (br s, 2H), 4.28-4.37 (m, 1H), 4.42-4.49 (m, 1H), 5.64-5.71 (m, 1H), 6.85 (t, J=7.3 Hz, 1H), 6.90 (d, J=8.1 Hz, 1H), 7.08-7.24 (m, 5H), 7.41 (dd, J=8.3, 1.9 Hz, 1H), 7.46-7.51 (m, 1H), 7.65-7.68 (m, 2H); LCMS (method C): Rt 1.18 min

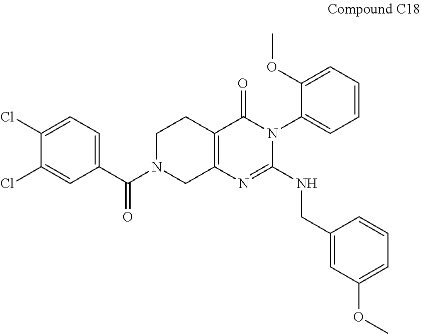 [SMILES: COc1cccc(CNc2nc3c(c(=O)n2-c2ccccc2OC)CCN(C(=O)c2ccc(Cl)c(Cl)c2)C3)c1]

¹H NMR (400 MHz, DMSO-d₆, 100° C.) δ ppm 2.40 (t, J=5.8 Hz, 2H), 3.56-3.67 (br s, 2H), 3.71 (s, 3H), 3.73 (s, 3H), 4.26 (br s, 2H), 4.31 (dd, J=15.2, 5.7 Hz, 1H), 4.45 (dd, J=15.4, 6.2 Hz, 1H), 6.07-6.14 (m, 1H), 6.71-6.81 (m, 3H), 7.05-7.12 (m, 1H), 7.12-7.19 (m, 2H), 7.21 (dd, J=8.4, 1.3 Hz, 1H), 7.41 (dd, J=8.3, 1.9 Hz, 1H), 7.47 (ddd, J=8.6, 7.5, 2.0 Hz, 1H), 7.65-7.69 (m, 2H); LCMS (method B): Rt 2.14 min

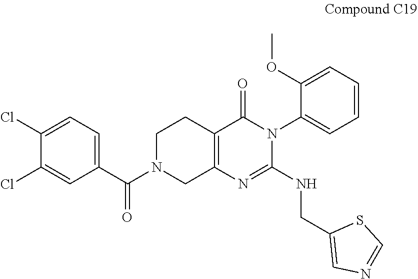 [SMILES: COc1ccccc1-n1c(NCc2cncs2)nc2c(c1=O)CCN(C(=O)c1ccc(Cl)c(Cl)c1)C2]

¹H NMR (400 MHz, DMSO-d₆, 100° C.) δ ppm 2.41 (t, J=5.9 Hz, 2H), 3.58-3.66 (m, 2H), 3.67 (s, 3H), 4.29-4.41 (m, 2H), 4.49-4.58 (m, 1H), 4.59-4.68 (m, 1H), 6.34 (br s, 1H), 7.04-7.14 (m, 2H), 7.20 (dd, J=8.5, 0.8 Hz, 1H), 7.42-7.49 (m, 2H), 7.65-7.71 (m, 3H), 8.82 (s, 1H); LCMS (method C): Rt 0.95 min

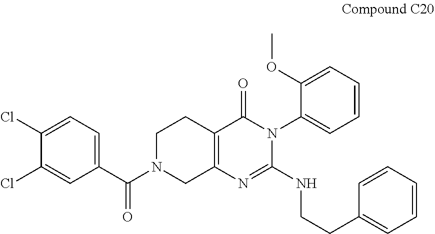 [SMILES: COc1ccccc1-n1c(NCCc2ccccc2)nc2c(c1=O)CCN(C(=O)c1ccc(Cl)c(Cl)c1)C2]

¹H NMR (400 MHz, DMSO-d₆, 100° C.) δ ppm 2.40 (t, J=5.8 Hz, 2H), 2.73 (t, J=7.2 Hz, 2H), 3.44 (q, J=6.7 Hz, 2H), 3.57-3.67 (m, 2H), 3.70 (s, 3H), 4.25-4.37 (m, 2H), 5.34 (br s, 1H), 7.03-7.23 (m, 8H), 7.41-7.48 (m, 2H), 7.67-7.70 (m, 2H); LCMS (method C): Rt 1.20 min

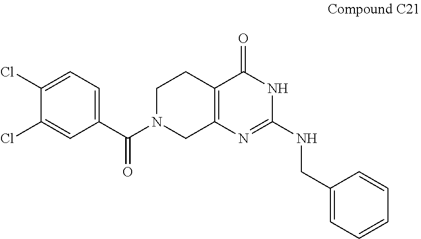 [SMILES: O=C(c1ccc(Cl)c(Cl)c1)N1CCc2c(nc(NCc3ccccc3)[nH]c2=O)C1]

¹H NMR (400 MHz, DMSO-d₆, 27° C.) δ ppm 2.35 (br s, 2H), 3.44 (br s, 1H), 3.77 (br s, 1H), 4.01-4.16 (m, 1H), 4.26-4.51 (m, 3H), 6.66-6.88 (m, 1H), 7.21-7.38 (m, 5H), 7.43 (br d, J=8.1 Hz, 1H), 7.69-7.75 (m, 2H), 10.81 (br s, 1H); LCMS (method B): Rt 1.87 min

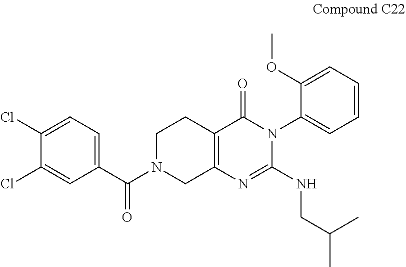 [SMILES: COc1ccccc1-n1c(NCC(C)C)nc2c(c1=O)CCN(C(=O)c1ccc(Cl)c(Cl)c1)C2]

¹H NMR (400 MHz, DMSO-d₆, 100° C.) δ ppm 0.76 (d, J=6.7 Hz, 6H), 1.79 (dt, J=13.5, 6.7 Hz, 1H), 2.40 (br t, J=5.9 Hz, 2H), 2.95-3.02 (m, 1H), 3.11 (dt, J=13.0, 6.6 Hz, 1H), 3.62 (br t, J=5.5 Hz, 2H), 3.74 (s, 3H), 4.22-4.36 (m, 2H), 5.22-5.33 (m, 1H), 7.06-7.12 (m, 1H), 7.15 (dd, J=7.7, 1.9 Hz, 1H), 7.22 (dd, J=8.3, 0.7 Hz, 1H), 7.43 (dd, J=8.1, 2.0 Hz, 1H), 7.45-7.51 (m, 1H), 7.66-7.70 (m, 2H); LCMS (method C): Rt 1.18 min

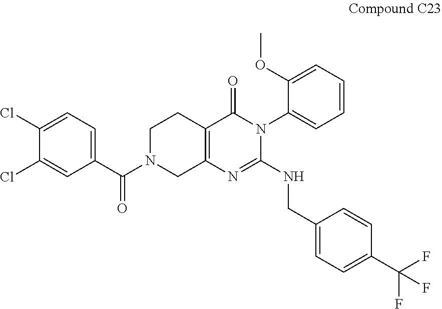 [SMILES: COc1ccccc1-n1c(NCc2ccc(C(F)(F)F)cc2)nc2c(c1=O)CCN(C(=O)c1ccc(Cl)c(Cl)c1)C2]

¹H NMR (400 MHz, DMSO-d₆, 100° C.) δ ppm 2.39 (t, J=5.9 Hz, 2H), 3.60 (br t, J=5.0 Hz, 2H), 3.75 (s, 3H), 4.23 (s, 2H), 4.41 (br d, J=15.5 Hz, 1H), 4.56 (br d, J=14.8 Hz, 1H), 6.35 (br s, 1H), 7.10 (td, J=7.6, 1.1 Hz, 1H), 7.19 (dd, J=7.8, 1.7 Hz, 1H), 7.22 (dd, J=8.5, 0.8 Hz, 1H), 7.39-7.50 (m, 4H), 7.59 (d, J=7.9 Hz, 2H), 7.63-7.68 (m, 2H); LCMS (method I): Rt 2.24 min

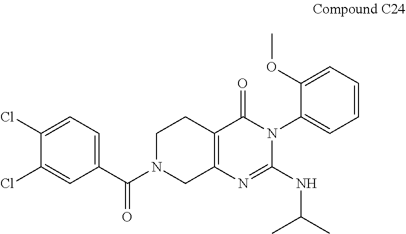 [SMILES: COc1ccccc1-n1c(NC(C)C)nc2c(c1=O)CCN(C(=O)c1ccc(Cl)c(Cl)c1)C2]

¹H NMR (400 MHz, DMSO-d₆, 100° C.) δ ppm 1.01-1.06 (m, 6H), 2.40 (t, J=5.8 Hz, 2H), 3.57-3.69 (m, 2H), 3.75 (s, 3H), 4.04-4.17 (m, 1H), 4.25-4.36 (m, 2H), 4.69-4.75 (m, 1H), 7.07-7.19 (m, 2H), 7.23 (d, J=8.1 Hz, 1H), 7.40-7.52 (m, 2H), 7.65-7.71 (m, 2H); LCMS (method C): Rt 1.13 min

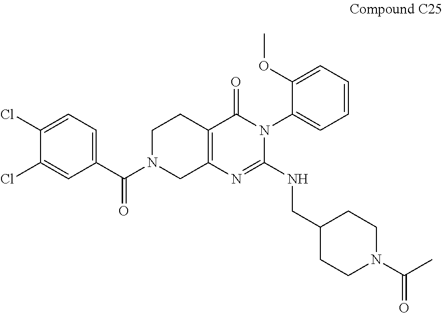 [SMILES: COc1ccccc1-n1c(NCC2CCN(C(C)=O)CC2)nc2c(c1=O)CCN(C(=O)c1ccc(Cl)c(Cl)c1)C2]

¹H NMR (400 MHz, DMSO-d₆, 100° C.) δ ppm 0.90-1.05 (m, 2H), 1.54 (br d, J=12.7 Hz, 2H), 1.69-1.81 (m, 1H), 1.93 (s, 3H), 2.40 (t, J=5.8 Hz, 2H), 2.54-2.87 (m, 2H), 3.01-3.10 (m, 1H), 3.19-3.29 (m, 1H), 3.62 (br t, J=5.0 Hz, 2H), 3.74 (s, 3H), 3.78-4.20 (m, 2H), 4.21-4.36 (m, 2H), 5.47 (br s, 1H), 7.09 (td, J=7.5, 1.1 Hz, 1H), 7.14 (dd, J=7.8, 1.9 Hz, 1H), 7.21 (dd, J=8.4, 1.2 Hz, 1H), 7.43 (dd, J=8.1, 2.0 Hz, 1H), 7.48 (ddd, J=8.4, 7.3, 2.0 Hz, 1H), 7.67-7.70 (m, 2H); LCMS (method A): Rt 1.76 min

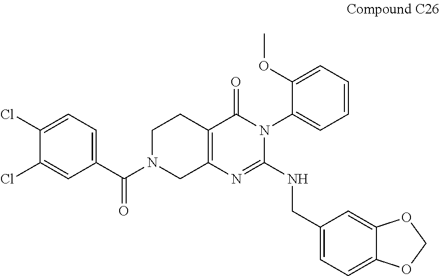 [SMILES: COc1ccccc1-n1c(NCc2ccc3c(c2)OCO3)nc2c(c1=O)CCN(C(=O)c1ccc(Cl)c(Cl)c1)C2]

¹H NMR (400 MHz, DMSO-d₆, 100° C.) δ ppm 2.39 (t, J=5.8 Hz, 2H), 3.54-3.68 (m, 2H), 3.73 (s, 3H), 4.19-4.30 (m, 3H), 4.39 (dd, J=15.0, 6.2 Hz, 1H), 5.91 (s, 2H), 6.01-6.10 (m, 1H), 6.67-6.78 (m, 3H), 7.09 (td, J=7.5, 1.1 Hz, 1H), 7.15 (dd, J=7.7, 1.8 Hz, 1H), 7.21 (dd, J=8.5, 1.2 Hz, 1H), 7.42 (dd, J=8.1, 1.9 Hz, 1H), 7.47 (ddd, J=9.0, 7.3, 1.8 Hz, 1H), 7.66 (d, J=0.4 Hz, 1H), 7.68 (d, J=5.5 Hz, 1H); LCMS (method C): Rt 1.12 min

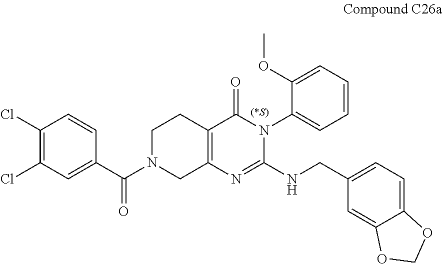 [SMILES: COc1ccccc1-n1c(NCc2ccc3c(c2)OCO3)nc2c(c1=O)CCN(C(=O)c1ccc(Cl)c(Cl)c1)C2]

¹H NMR (400 MHz, DMSO-d₆, 80° C.) δ ppm 2.39 (t, J=5.8 Hz, 2H), 3.61 (br s, 2H), 3.73 (s, 3H), 4.18-4.31 (m, 3H), 4.38 (dd, J=15.0, 5.9 Hz, 1H), 5.92 (s, 2H), 6.14-6.25 (m, 1H), 6.65-6.72 (m, 1H), 6.73-6.79 (m, 2H), 7.05-7.12 (m, 1H), 7.16 (dd, J=7.7, 1.5 Hz, 1H), 7.22 (d, J=8.4 Hz, 1H), 7.43 (dd, J=8.3, 1.9 Hz, 1H), 7.45-7.52 (m, 1H), 7.65-7.72 (m, 2H); LCMS (method D): Rt 2.02 min

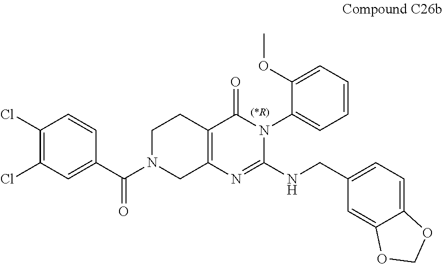 [SMILES: COc1ccccc1-n1c(NCc2ccc3c(c2)OCO3)nc2c(c1=O)CCN(C(=O)c1ccc(Cl)c(Cl)c1)C2]

¹H NMR (400 MHz, DMSO-d₆, 80° C.) δ ppm 2.39 (t, J=5.7 Hz, 2H), 3.61 (br s, 2H), 3.73 (s, 3H), 4.17-4.32 (m, 3H), 4.39 (dd, J=15.1, 6.1 Hz, 1H), 5.92 (s, 2H), 6.15-6.26 (m, 1H), 6.64-6.80 (m, 3H), 7.09 (td, J=7.7, 1.1 Hz, 1H), 7.16 (dd, J=7.7, 1.8 Hz, 1H), 7.22 (dd, J=8.5, 1.0 Hz, 1H), 7.43 (dd, J=8.5, 2.0 Hz, 1H), 7.48 (ddd, J=8.3, 7.4, 1.8 Hz, 1H), 7.66-7.71 (m, 2H); LCMS (method D): Rt 2.01 min

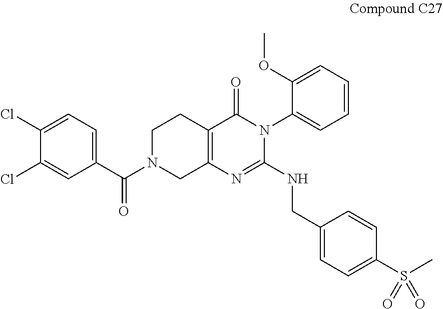 [SMILES: COc1ccccc1-n1c(NCc2ccc(S(C)(=O)=O)cc2)nc2c(c1=O)CCN(C(=O)c1ccc(Cl)c(Cl)c1)C2]

¹H NMR (400 MHz, DMSO-d₆, 100° C.) δ ppm 2.39 (t, J=5.8 Hz, 2H), 3.11 (s, 3H), 3.56-3.64 (m, 2H), 3.76 (s, 3H), 4.24 (s, 2H), 4.43 (dd, J=15.8, 5.7 Hz, 1H), 4.59 (dd, J=15.8, 6.3 Hz, 1H), 6.37 (br t, J=5.7 Hz, 1H), 7.10 (td, J=7.5, 1.3 Hz, 1H), 7.23 (dd, J=7.7, 1.8 Hz, 1H), 7.26 (dd, J=8.4, 1.1 Hz, 1H), 7.40 (dd, J=8.1, 2.0 Hz, 1H), 7.43-7.51 (m, 3H), 7.64-7.68 (m, 2H), 7.79-7.84 (m, 2H); LCMS (method C): Rt 0.99 min

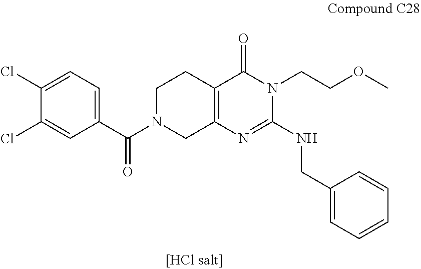 [SMILES: COCCn1c(NCc2ccccc2)nc2c(c1=O)CCN(C(=O)c1ccc(Cl)c(Cl)c1)C2]

¹H NMR (400 MHz, DMSO-d₆, 81° C.) δ ppm 2.39 (t, J=5.8 Hz, 2H), 3.23 (s, 3H), 3.55 (t, J=5.6 Hz, 2H), 4.16 (t, J=5.5 Hz, 2H), 4.15-4.25 (m, 2H), 4.30-4.43 (m, 2H), 4.50 (br d, J=5.3 Hz, 2H), 7.19-7.35 (m, 6H), 7.39 (dd, J=8.4, 2.0 Hz, 1H), 7.65 (d, J=2.0 Hz, 1H), 7.67 (d, J=8.1 Hz, 1H); LCMS (method B): Rt 2.16 min

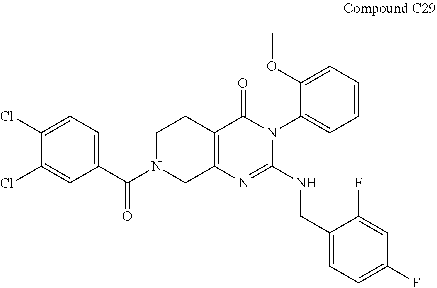 [SMILES: COc1ccccc1-n1c(NCc2ccc(F)cc2F)nc2c(c1=O)CCN(C(=O)c1ccc(Cl)c(Cl)c1)C2]

¹H NMR (400 MHz, DMSO-d₆, 100° C.) δ ppm 2.39 (t, J=5.9 Hz, 2H), 3.56-3.64 (m, 2H), 3.75 (s, 3H), 4.24 (br s, 2H), 4.33 (dd, J=15.3, 5.4 Hz, 1H), 4.48 (dd, J=15.4, 5.9 Hz, 1H), 6.15-6.22 (m, 1H), 6.93-7.03 (m, 2H), 7.09 (td, J=7.7, 1.1 Hz, 1H), 7.18 (dd, J=7.7, 1.8 Hz, 1H), 7.22 (dd, J=8.4, 0.9 Hz, 1H), 7.25-7.34 (m, 1H), 7.41 (dd, J=8.1, 2.0 Hz, 1H), 7.48 (ddd, J=9.1, 7.5, 1.8 Hz, 1H), 7.67-7.70 (m, 2H); LCMS (method B): Rt 2.16 min

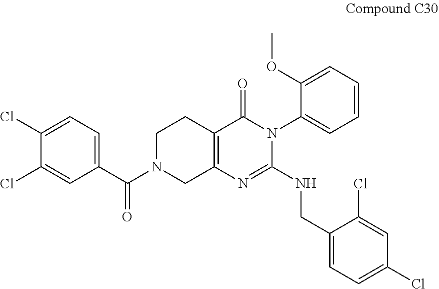 [SMILES: COc1ccccc1-n1c(NCc2ccc(Cl)cc2Cl)nc2c(c1=O)CCN(C(=O)c1ccc(Cl)c(Cl)c1)C2]

¹H NMR (400 MHz, DMSO-d₆, 100° C.) δ ppm 2.37-2.45 (m, 2H), 3.55-3.67 (m, 2H), 3.76-3.81 (m, 3H), 4.22 (br s, 2H), 4.33-4.41 (m, 1H), 4.49-4.58 (m, 1H), 6.18-6.27 (m, 1H), 7.08-7.14 (m, 1H), 7.20-7.35 (m, 4H), 7.37-7.51 (m, 3H), 7.64-7.69 (m, 2H); LCMS (method C): Rt 1.29 min

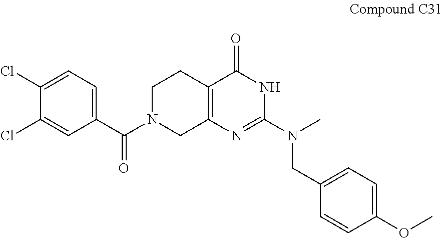 [SMILES: COc1ccc(CN(C)c2nc3c(c(=O)[nH]2)CCN(C(=O)c2ccc(Cl)c(Cl)c2)C3)cc1]

¹H NMR (400 MHz, DMSO-d₆, 125° C.) δ ppm 2.41 (t, J=5.9 Hz, 2H), 2.95 (s, 3H), 3.61 (t, J=5.8 Hz, 2H), 3.74 (s, 3H), 4.26 (s, 2H), 4.64 (s, 2H), 6.83-6.89 (m, 2H), 7.13-7.20 (m, 2H), 7.39 (dd, J=8.4, 2.0 Hz, 1H), 7.62 (d, J=1.8 Hz, 1H), 7.65 (d, J=8.4 Hz, 1H), 10.55 (br s, 1H); LCMS (method B): Rt 1.96 min

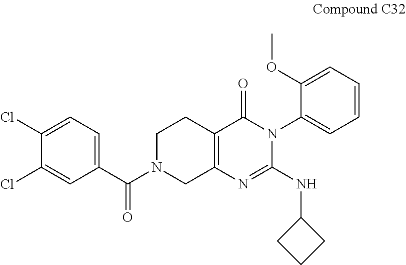 [SMILES: COc1ccccc1-n1c(NC2CCC2)nc2c(c1=O)CCN(C(=O)c1ccc(Cl)c(Cl)c1)C2]

¹H NMR (400 MHz, DMSO-d₆, 100° C.) δ ppm 1.52-1.63 (m, 2H), 1.75-1.91 (m, 2H), 2.09-2.21 (m, 2H), 2.39 (t, J=5.8 Hz, 2H), 3.57-3.68 (m, 2H), 3.76 (s, 3H), 4.22-4.40 (m, 3H), 5.30 (br d, J=6.4 Hz, 1H), 7.06-7.11 (m, 1H), 7.12-7.16 (m, 1H), 7.22 (dd, J=8.4, 1.0 Hz, 1H), 7.43 (dd, J=8.1, 2.0 Hz, 1H), 7.48 (ddd, J=8.4, 7.3, 1.9 Hz, 1H), 7.67 (s, 1H), 7.69 (d, J=6.6 Hz, 1H); LCMS (method C): Rt 1.16 min

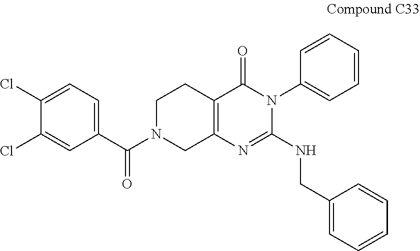 [SMILES: O=C(c1ccc(Cl)c(Cl)c1)N1CCc2c(nc(NCc3ccccc3)n(-c3ccccc3)c2=O)C1]

¹H NMR (400 MHz, DMSO-d₆, 100° C.) δ ppm 2.41 (t, J=5.9 Hz, 2H), 3.62 (br t, J=5.3 Hz, 2H), 4.26 (s, 2H), 4.42 (d, J=5.9 Hz, 2H), 5.96-6.05 (m, 1H), 7.14-7.28 (m, 7H), 7.40 (dd, J=8.1, 2.0 Hz, 1H), 7.46-7.58 (m, 3H), 7.64-7.69 (m, 2H); LCMS (method B): Rt 2.16 min

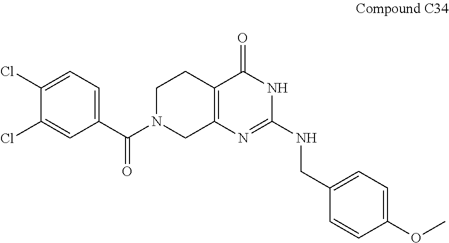 [SMILES: COc1ccc(CNc2nc3c(c(=O)[nH]2)CCN(C(=O)c2ccc(Cl)c(Cl)c2)C3)cc1]

¹H NMR (400 MHz, DMSO-d₆, 27° C.) δ ppm 2.34 (br s, 2H), 3.44 (br s, 1H), 3.65-3.83 (m, 1H), 3.73 (s, 3H), 4.09 (br s, 1H), 4.24-4.44 (m, 3H), 6.55-6.79 (m, 1H), 6.89 (br s, 2H), 7.13-7.32 (m, 2H), 7.44 (br d, J=7.9 Hz, 1H), 7.73 (d, J=8.1 Hz, 2H), 10.74 (br s, 1H); LCMS (method C): Rt 0.97 min

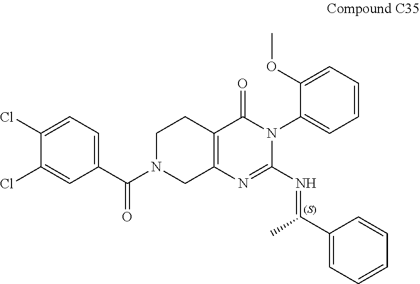

¹H NMR (400 MHz, DMSO-d₆, 100° C.) δ ppm 1.31-1.37 (m, 3H), 2.35-2.44 (m, 2H), 3.54-3.67 (m, 2H), 3.68-3.82 (m, 3H), 4.15-4.37 (m, 2H), 5.13-5.24 (m, 1H), 5.31 (br d, J=6.8 Hz, 1H), 7.06-7.30 (m, 8H), 7.38-7.43 (m, 1H), 7.46-7.53 (m, 1H), 7.63-7.71 (m, 2H); LCMS (method C): Rt 1.21 min

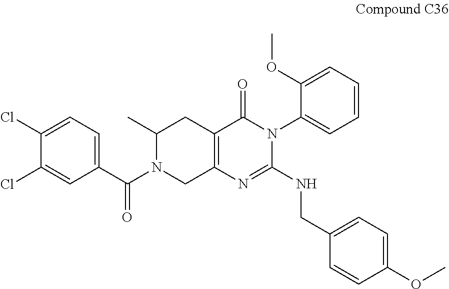 [SMILES: COc1ccc(CNc2nc3c(c(=O)n2-c2ccccc2OC)CC(C)N(C(=O)c2ccc(Cl)c(Cl)c2)C3)cc1]

¹H NMR (400 MHz, DMSO-d₆, 100° C.) δ ppm 1.13-1.20 (m, 3H), 2.31 (br d, J=16.0 Hz, 1H), 2.53 (br dd, J=16.3, 5.9 Hz, 1H), 3.71 (s, 3H), 3.73 (m, 3H), 3.91-4.03 (m, 1H), 4.20-4.66 (m, 4H), 5.96 (br s, 1H), 6.78-6.84 (m, 2H), 7.05-7.23 (m, 5H), 7.36-7.42 (m, 1H), 7.46 (ddd, J=9.0, 7.5, 1.8 Hz, 1H), 7.61-7.72 (m, 2H); LCMS (method C): Rt 1.17 min

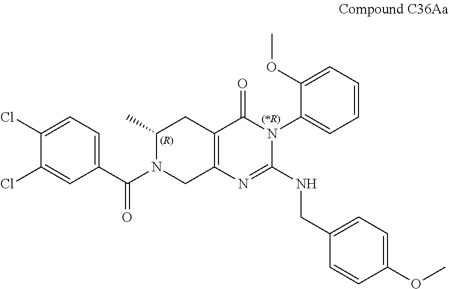

¹H NMR (400 MHz, DMSO-d₆, 80° C.) δ ppm 1.16 (d, J=6.8 Hz, 3H), 2.30 (br d, J=16.3 Hz, 1H), 2.50-2.59 (m, 1H), 3.71 (s, 3H), 3.72 (s, 3H), 3.96 (br d, J=18.7 Hz, 1H), 4.30-4.62 (m, 3H), 4.24 (dd, J=15.0, 5.5 Hz, 1H), 6.12 (br t, J=4.7 Hz, 1H), 6.79-6.85 (m, 2H), 7.08 (td, J=7.7, 1.3 Hz, 1H), 7.14 (d, J=8.8 Hz, 2H), 7.17-7.24 (m, 2H), 7.41 (dd, J=8.1, 2.0 Hz, 1H), 7.47 (ddd, J=8.4, 7.5, 1.8 Hz, 1H), 7.65-7.72 (m, 2H); LCMS (method D): Rt 2.10 min

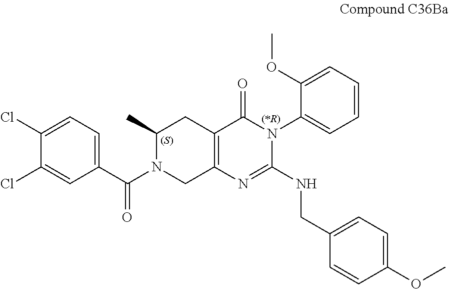

¹H NMR (400 MHz, DMSO-d₆, 80° C.) δ ppm 1.17 (d, J=6.8 Hz, 3H), 2.31 (d, J=16.5 Hz, 1H), 2.50-2.58 (m, 1H), 3.71 (s, 3H), 3.74 (s, 3H), 3.98 (br d, J=18.1 Hz, 1H), 4.27-4.58 (m, 4H), 6.12 (br t, J=5.5 Hz, 1H), 6.79-6.83 (m, 2H), 7.08 (td, J=7.5, 1.1 Hz, 1H), 7.11-7.18 (m, 3H), 7.21 (dd, J=8.4, 0.9 Hz, 1H), 7.40 (dd, J=8.1, 2.0 Hz, 1H), 7.47 (ddd, J=9.2, 7.4, 1.9 Hz, 1H), 7.66 (d, J=2.0 Hz, 1H), 7.69 (d, J=8.1 Hz, 1H); LCMS (method D): Rt 2.11 min

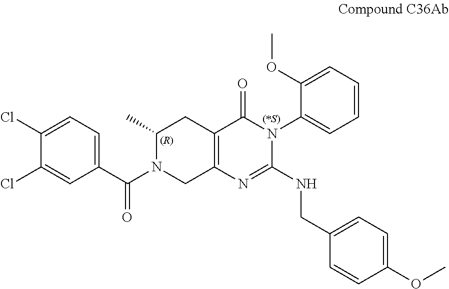

¹H NMR (400 MHz, DMSO-d₆, 80° C.) δ ppm 1.17 (d, J=6.8 Hz, 3H), 2.31 (br d, J=16.3 Hz. 1H). 2.49-2.58 (m. 1H). 3.71 (s. 3H). 3.74 (s. 3H). 3.98 (br d. J=18.5 Hz. 1H). 4.17-4.69 (m, 4H), 6.06-6.15 (m, 1H), 6.78-6.85 (m, 2H), 7.08 (td, J=7.7, 1.1 Hz, 1H), 7.11-7.17 (m, 3H), 7.21 (dd, J=8.4, 0.9 Hz, 1H), 7.40 (dd, J=8.3, 1.9 Hz, 1H), 7.47 (ddd, J=9.0, 7.3, 1.8 Hz, 1H), 7.66 (d, J=2.0 Hz, 1H), 7.69 (d, J=8.1 Hz, 1H); LCMS (method D): Rt 2.10 min

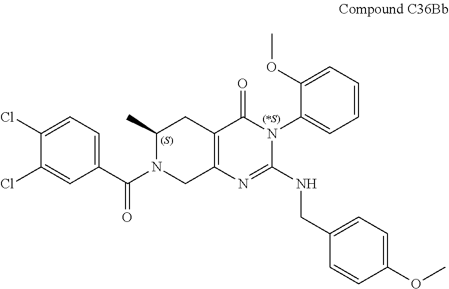

¹H NMR (400 MHz, DMSO-d₆, 80° C.) δ ppm 1.16 (d, J=6.8 Hz, 3H), 2.30 (br d, J=16.3 Hz, 1H), 2.49-2.58 (m, 1H), 3.71 (s, 3H), 3.72 (s, 3H), 3.96 (br d, J=19.6 Hz, 1H), 4.24 (dd, J=15.0, 5.5 Hz, 1H), 4.31-4.60 (m, 3H), 6.12 (br t, J=5.6 Hz, 1H), 6.79-6.84 (m, 2H), 7.08 (td, J=7.5, 1.1 Hz, 1H), 7.13 (d, J=8.6 Hz, 2H), 7.17-7.23 (m, 2H), 7.41 (dd, J=8.4, 2.0 Hz, 1H), 7.47 (ddd, J=8.4, 7.5, 1.8 Hz, 1H), 7.65-7.72 (m, 2H); LCMS (method D): Rt 2.10 min

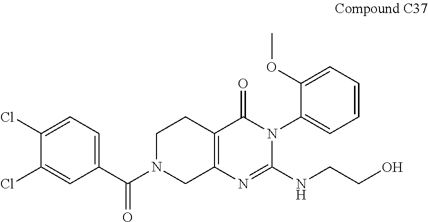 [SMILES: COc1ccccc1-n1c(NCCO)nc2c(c1=O)CCN(C(=O)c1ccc(Cl)c(Cl)c1)C2]

¹H NMR (400 MHz, DMSO-d₆, 125° C.) δ ppm 2.41 (br t, J=5.8 Hz, 2H), 3.26-3.33 (m, 2H), 3.37-3.49 (m, 2H), 3.63 (br t, J=5.9 Hz, 2H), 3.75 (s, 3H), 4.11 (br s, 1H), 4.24-4.38 (m, 2H), 5.17-5.28 (m, 1H), 7.09 (td, J=7.5, 1.2 Hz, 1H), 7.15 (dd, J=7.8, 1.9 Hz, 1H), 7.21 (dd, J=8.4, 1.1 Hz, 1H), 7.42 (dd, J=8.1, 1.2 Hz, 1H), 7.47 (ddd, J=8.3, 7.4, 1.9 Hz, 1H), 7.66 (s, 1H), 7.67 (d, J=6.6 Hz, 1H); LCMS (method B): Rt 1.65 min

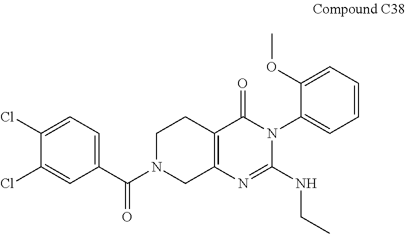 [SMILES: CCNc1nc2c(c(=O)n1-c1ccccc1OC)CCN(C(=O)c1ccc(Cl)c(Cl)c1)C2]

¹H NMR (400 MHz, DMSO-d₆, 80° C.) δ ppm 0.99 (t, J=7.0 Hz, 3H), 2.39 (t, J=5.9 Hz, 2H), 3.13-3.31 (m, 2H), 3.49-3.72 (m, 2H), 3.74 (s, 3H), 4.15-4.52 (m, 2H), 5.59 (br t, J=4.5 Hz, 1H), 7.04-7.15 (m, 2H), 7.21 (dd, J=8.4, 1.1 Hz, 1H), 7.42-7.51 (m, 2H), 7.68-7.72 (m, 2H); LCMS (method B): Rt 1.95 min

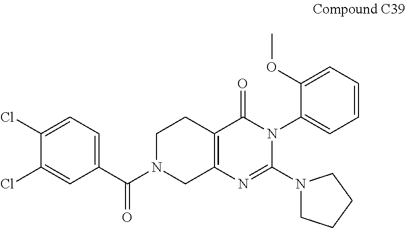 [SMILES: COc1ccccc1-n1c(N2CCCC2)nc2c(c1=O)CCN(C(=O)c1ccc(Cl)c(Cl)c1)C2]

¹H NMR (400 MHz, DMSO-d₆, 125° C.) δ ppm 1.58-1.67 (m, 4H), 2.41 (br t, J=5.9 Hz, 2H), 2.93-3.00 (m, 4H), 3.63 (t, J=5.9 Hz, 2H), 3.76 (s, 3H), 4.22-4.37 (m, 2H), 7.01 (td, J=7.6, 1.1 Hz, 1H), 7.13 (dd, J=8.4, 1.1 Hz, 1H), 7.17 (dd, J=7.7, 1.8 Hz, 1H), 7.37-7.44 (m, 2H), 7.64-7.69 (m, 2H); LCMS (method C): Rt 1.16 min

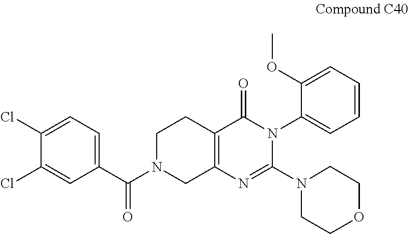 [SMILES: COc1ccccc1-n1c(N2CCOCC2)nc2c(c1=O)CCN(C(=O)c1ccc(Cl)c(Cl)c1)C2]

¹H NMR (400 MHz, DMSO-d₆, 125° C.) δ ppm 2.41-2.47 (m, 2H), 2.89-3.06 (m, 4H), 3.22-3.34 (m, 4H), 3.59-3.69 (m, 2H), 3.77 (s, 3H), 4.30-4.42 (m, 2H), 7.04 (td, J=7.6, 1.3 Hz, 1H), 7.17 (dd, J=8.4, 1.3 Hz, 1H), 7.24 (dd, J=7.8, 1.7 Hz, 1H), 7.38-7.45 (m, 2H), 7.66 (s, 1H), 7.67 (d, J=5.5 Hz, 1H); LCMS (method C): Rt 1.02 min

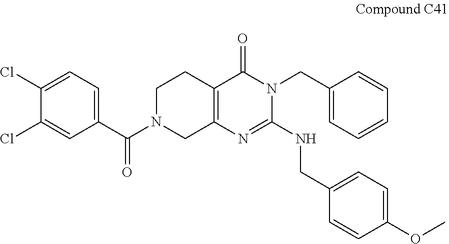 [SMILES: COc1ccc(CNc2nc3c(c(=O)n2Cc2ccccc2)CCN(C(=O)c2ccc(Cl)c(Cl)c2)C3)cc1]

¹H NMR (400 MHz, CDCl₃, 57° C.) δ ppm 2.66 (br t, J=5.4 Hz, 2H), 3.51-3.79 (m, 2H), 3.77 (s, 3H), 4.35 (br d, J=4.6 Hz, 2H), 4.21-4.55 (m, 2H), 4.65 (br t, J=4.3 Hz, 1H), 5.20 (s, 2H), 6.73-6.78 (m, 2H), 6.90 (br d, J=8.6 Hz, 2H), 7.15-7.19 (m, 2H), 7.26-7.34 (m, 4H), 7.50 (d, J=8.1 Hz, 1H), 7.56 (d, J=2.0 Hz, 1H); LCMS (method C): 1.21 min

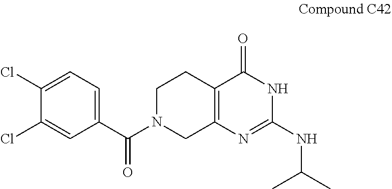 [SMILES: CC(C)Nc1nc2c(c(=O)[nH]1)CCN(C(=O)c1ccc(Cl)c(Cl)c1)C2]

¹H NMR (400 MHz, DMSO-d₆, 125° C.) δ ppm 1.13 (d, J=6.6 Hz, 6H), 2.37 (t, J=5.8 Hz, 2H), 3.58 (t, J=5.8 Hz, 2H), 3.89-4.03 (m, 1H), 4.21 (s, 2H), 5.89 (br s, 1H), 7.38 (dd, J=8.1, 2.0 Hz, 1H), 7.62 (d, J=2.0 Hz, 1H), 7.65 (d, J=8.4 Hz, 1H), 10.11 (br s, 1H); LCMS (method B): Rt 1.71 min

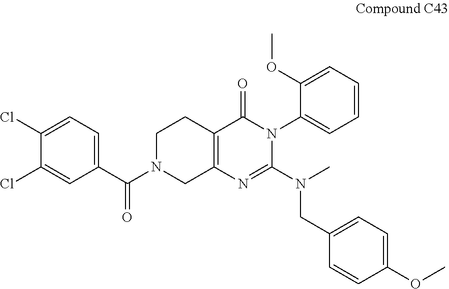 [SMILES: COc1ccc(CN(C)c2nc3c(c(=O)n2-c2ccccc2OC)CCN(C(=O)c2ccc(Cl)c(Cl)c2)C3)cc1]

¹H NMR (400 MHz, DMSO-d₆, 100° C.) δ ppm 2.34 (s, 3H), 2.45 (br t, J=5.9 Hz, 2H), 3.65 (br t, J=4.7 Hz, 2H), 3.71 (s, 3H), 3.74 (s, 3H), 4.19 (q, J=14.7 Hz, 2H), 4.29-4.43 (m, 2H), 6.75-6.82 (m, 2H), 6.92-6.98 (m, 2H), 7.01 (td, J=7.6, 1.3 Hz, 1H), 7.13 (dd, J=8.4, 1.1 Hz, 1H), 7.20 (dd, J=7.8, 1.7 Hz, 1H), 7.37 (ddd, J=8.3, 7.4, 1.7 Hz, 1H), 7.44 (dd, J=8.2, 1.9 Hz, 1H), 7.65-7.72 (m, 2H); LCMS (method C): Rt 1.23 min

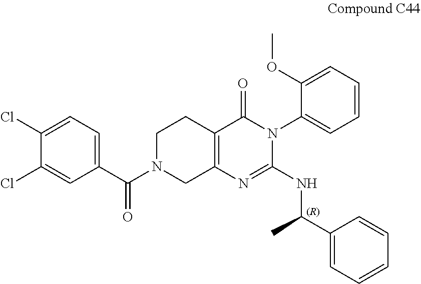

¹H NMR (400 MHz, DMSO-d₆, 100° C.) δ ppm 1.32-1.37 (m, 3H), 2.36-2.42 (m, 2H), 3.54-3.67 (m, 2H), 3.68-3.83 (m, 3H), 4.15-4.37 (m, 2H), 5.13-5.24 (m, 1H), 5.27-5.36 (m, 1H), 7.06-7.29 (m, 8H), 7.39-7.43 (m, 1H), 7.46-7.52 (m, 1H), 7.64-7.70 (m, 2H); LCMS (method C): Rt 1.22 min

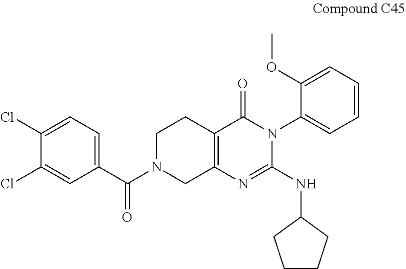 [SMILES: COc1ccccc1-n1c(NC2CCCC2)nc2c(c1=O)CCN(C(=O)c1ccc(Cl)c(Cl)c1)C2]

¹H NMR (400 MHz, DMSO-d₆, 80° C.) δ ppm 1.25-1.36 (m, 2H), 1.40-1.55 (m, 4H), 1.74-1.89 (m, 2H), 2.40 (br t, J=5.8 Hz, 2H), 3.63 (br s, 2H), 3.75 (s, 3H), 4.15-4.24 (m, 1H), 4.31 (br s, 2H), 4.82 (br d, J=7.5 Hz, 1H), 7.06-7.13 (m, 1H), 7.13-7.18 (m, 1H), 7.20-7.26 (m, 1H), 7.41-7.52 (m, 2H), 7.66-7.73 (m, 2H); LCMS (method C): Rt 1.21 min

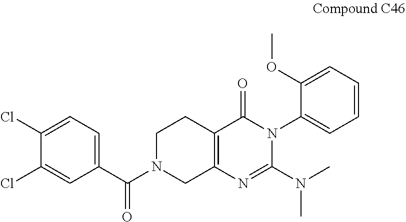 [SMILES: COc1ccccc1-n1c(N(C)C)nc2c(c1=O)CCN(C(=O)c1ccc(Cl)c(Cl)c1)C2]

¹H NMR (400 MHz, DMSO-d₆, 150° C.) δ ppm 2.43 (tt, J=5.9, 1.4 Hz, 2H), 2.57 (s, 6H), 3.64 (td, J=5.9, 1.5 Hz, 2H), 3.76 (s, 3H), 4.27-4.38 (m, 2H), 7.02 (td, J=7.6, 1.3 Hz, 1H), 7.14 (dd, J=8.4, 1.3 Hz, 1H), 7.17 (dd, J=7.7, 1.8 Hz, 1H), 7.35-7.44 (m, 2H), 7.60-7.71 (m, 2H); LCMS (method B): Rt 2.04 min

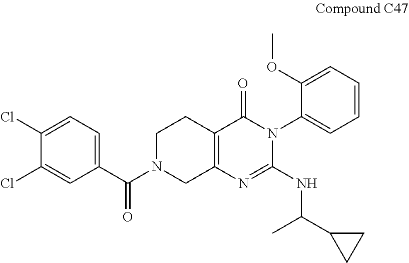 [SMILES: COc1ccccc1-n1c(NC(C)C2CC2)nc2c(c1=O)CCN(C(=O)c1ccc(Cl)c(Cl)c1)C2]

¹H NMR (400 MHz, DMSO-d₆, 80° C.) δ ppm 0.07-0.42 (m, 4H), 0.79-0.93 (m, 1H), 1.09 (dd, J=6.5, 2.5 Hz, 3H), 2.39 (br t, J=5.8 Hz, 2H), 3.42-3.54 (m, 1H), 3.54-3.70 (m, 2H), 3.77 (d, J=3.5 Hz, 3H), 4.17-4.41 (m, 2H), 4.89 (dd, J=20.9, 7.9 Hz, 1H), 7.11 (td, J=7.5, 1.5 Hz, 1H), 7.17 (dt, J=7.7, 1.5 Hz, 1H), 7.24 (dd, J=8.4, 0.9 Hz, 1H), 7.44 (dd, J=8.3, 1.9 Hz, 1H), 7.50 (ddd, J=8.6, 7.1, 1.9 Hz, 1H), 7.66-7.73 (m, 2H); LCMS (method B): Rt 2.19 min

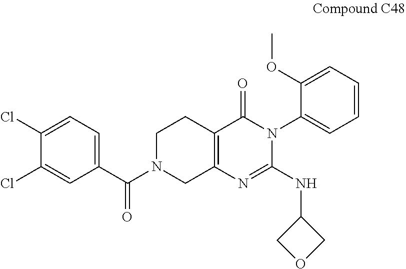 [SMILES: COc1ccccc1-n1c(NC2COC2)nc2c(c1=O)CCN(C(=O)c1ccc(Cl)c(Cl)c1)C2]

¹H NMR (400 MHz, DMSO-d₆, 80° C.) δ ppm 2.39 (br t, J=5.8 Hz, 2H), 3.48-3.71 (m, 2H), 3.76 (s, 3H), 4.28 (br s, 2H), 4.38 (dt, J=15.6, 6.5 Hz, 2H), 4.60 (td, J=6.7, 2.8 Hz, 2H), 4.75-4.89 (m, 1H), 6.25 (br s, 1H), 7.06-7.12 (m, 1H), 7.17 (dd, J=7.7, 1.8 Hz, 1H), 7.23 (dd, J=8.5, 1.0 Hz, 1H), 7.43 (dd, J=8.3, 2.1 Hz, 1H), 7.49 (ddd, J=9.0, 7.5, 1.8 Hz, 1H), 7.69 (s, 1H), 7.70 (d, J=6.4 Hz, 1H); LCMS (method B): Rt 1.77 min

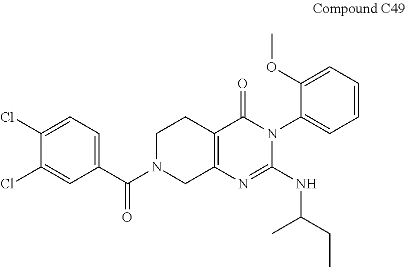 [SMILES: CCC(C)Nc1nc2c(c(=O)n1-c1ccccc1OC)CCN(C(=O)c1ccc(Cl)c(Cl)c1)C2]

¹H NMR (400 MHz, DMSO-d₆, 100° C.) δ ppm 0.74 (td, J=7.5, 2.6 Hz, 3H), 1.01 (dd, J=6.5, 1.7 Hz, 3H), 1.31-1.44 (m, 2H), 2.40 (t, J=5.9 Hz, 2H), 3.63 (br t, J=4.6 Hz, 2H), 3.75 (d, J=4.4 Hz, 3H), 3.87-3.99 (m, 1H), 4.21-4.38 (m, 2H), 4.63 (br d, J=7.5 Hz, 1H), 7.07-7.12 (m, 1H), 7.13-7.18 (m, 1H), 7.20-7.25 (m, 1H), 7.41-7.45 (m, 1H), 7.46-7.52 (m, 1H), 7.67 (s, 1H), 7.69 (d, J=5.9 Hz, 1H); LCMS (method A): Rt 2.17 min

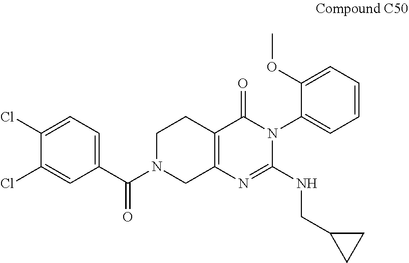 [SMILES: COc1ccccc1-n1c(NCC2CC2)nc2c(c1=O)CCN(C(=O)c1ccc(Cl)c(Cl)c1)C2]

¹H NMR (400 MHz, DMSO-d₆, 150° C.) δ ppm 0.05-0.17 (m, 2H), 0.28-0.38 (m, 2H), 0.90-1.03 (m, 1H), 2.42 (t, J=5.8 Hz, 2H), 3.02-3.12 (m, 1H), 3.13-3.23 (m, 1H), 3.64 (t, J=5.9 Hz, 2H), 3.76 (s, 3H), 4.23-4.35 (m, 2H), 5.10 (br s, 1H), 7.09 (td, J=7.6, 1.1 Hz, 1H), 7.14 (dd, J=7.8, 1.9 Hz, 1H), 7.21 (dd, J=8.4, 1.1 Hz, 1H), 7.41 (dd, J=8.3, 1.9 Hz, 1H), 7.47 (ddd, J=8.6, 7.0, 1.9 Hz, 1H), 7.61-7.68 (m, 2H); LCMS (method B): Rt 2.09 min

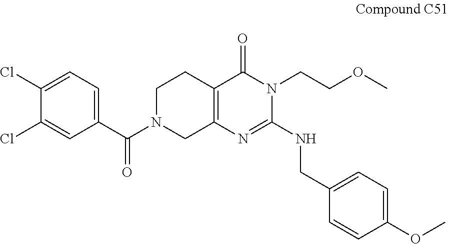 [SMILES: COCCn1c(NCc2ccc(OC)cc2)nc2c(c1=O)CCN(C(=O)c1ccc(Cl)c(Cl)c1)C2]

¹H NMR (400 MHz, CDCl₃, 57° C.) δ ppm 2.58 (br t, J=5.4 Hz, 2H), 3.23 (s, 3H), 3.58-3.85 (m, 2H), 3.65 (br t, J=4.4 Hz, 2H), 3.79 (s, 3H), 4.15 (br t, J=4.4 Hz, 2H), 4.28-4.59 (m, 2H), 4.43 (br d, J=4.6 Hz, 2H), 6.43 (br t, J=4.5 Hz, 1H), 6.82-6.92 (m, 2H), 7.19-7.29 (m, 3H), 7.49 (d, J=8.1 Hz, 1H), 7.55 (d, J=2.0 Hz, 1H); LCMS (method C): Rt1.14 min

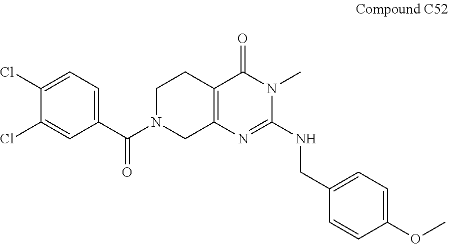 [SMILES: COc1ccc(CNc2nc3c(c(=O)n2C)CCN(C(=O)c2ccc(Cl)c(Cl)c2)C3)cc1]

¹H NMR (400 MHz, CDCl₃, 57° C.) δ ppm 2.59 (br t, J=5.5 Hz, 2H), 3.36 (s, 3H), 3.60-3.71 (m, 2H), 3.80 (s, 3H), 4.40 (br s, 2H), 4.52 (br d, J=4.8 Hz, 2H), 4.67-4.76 (m, 1H), 6.85-6.91 (m, 2H), 7.22-7.30 (m, 3H), 7.50 (d, J=8.1 Hz, 1H), 7.55 (d, J=2.0 Hz, 1H); LCMS (method C): Rt 1.05 min

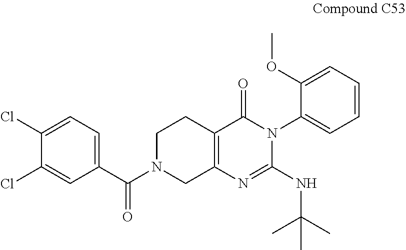 [SMILES: COc1ccccc1-n1c(NC(C)(C)C)nc2c(c1=O)CCN(C(=O)c1ccc(Cl)c(Cl)c1)C2]

¹H NMR (400 MHz, DMSO-d₆, 80° C.) δ ppm 1.26 (s, 9H), 2.41 (br t, J=5.9 Hz, 2H), 3.62 (br s, 2H), 3.78 (s, 3H), 4.13 (s, 1H), 4.22-4.52 (m, 2H), 7.12 (td, J=7.7, 1.1 Hz, 1H), 7.20 (dd, J=7.9, 1.8 Hz, 1H), 7.26 (dd, J=8.4, 1.1 Hz, 1H), 7.45 (dd, J=8.2, 1.9 Hz, 1H), 7.51 (ddd, J=8.4, 7.5, 1.8 Hz, 1H), 7.67-7.74 (m, 2H); LCMS (method A): Rt 2.23 min

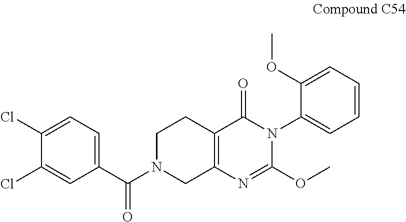 [SMILES: COc1ccccc1-n1c(OC)nc2c(c1=O)CCN(C(=O)c1ccc(Cl)c(Cl)c1)C2]

¹H NMR (400 MHz, DMSO-d₆, 80° C.) δ ppm 2.41-2.48 (m, 2H), 3.56-3.72 (m, 2H), 3.73 (s, 3H), 3.79 (s, 3H), 4.30-4.52 (m, 2H), 7.03 (td, J=7.6, 1.2 Hz, 1H), 7.14-7.21 (m, 2H), 7.41 (ddd, J=8.4, 7.5, 1.8 Hz, 1H), 7.46 (dd, J=8.4, 2.0 Hz, 1H), 7.66-7.76 (m, 2H); LCMS (method A): Rt 1.98 min

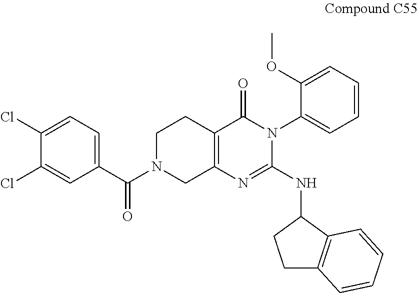 [SMILES: COc1ccccc1-n1c(NC2CCc3ccccc32)nc2c(c1=O)CCN(C(=O)c1ccc(Cl)c(Cl)c1)C2]

¹H NMR (400 MHz, DMSO-d₆, 80° C.) δ ppm 1.73-1.89 (m, 1H), 2.29-2.40 (m, 1H), 2.43 (br t, J=5.7 Hz, 2H), 2.69-2.90 (m, 2H), 3.65 (br s, 2H), 3.75 (d, J=1.5 Hz, 3H), 4.15-4.54 (m, 2H), 5.47-5.69 (m, 2H), 7.00-7.25 (m, 7H), 7.38-7.49 (m, 2H), 7.65-7.73 (m, 2H); LCMS (method B): Rt 2.31 min

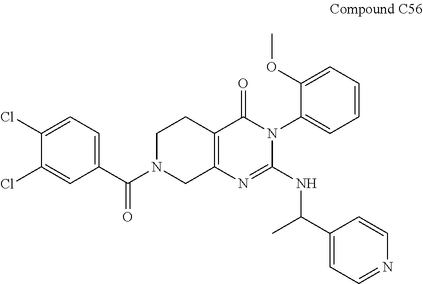 [SMILES: COc1ccccc1-n1c(NC(C)c2ccncc2)nc2c(c1=O)CCN(C(=O)c1ccc(Cl)c(Cl)c1)C2]

¹H NMR (400 MHz, DMSO-d₆, 80° C.) δ ppm 1.29-1.42 (m, 3H), 2.32-2.43 (m, 2H), 3.50-3.73 (m, 2H), 3.78 (d, J=7.7 Hz, 3H), 4.08-4.36 (m, 2H), 5.12-5.27 (m, 1H), 5.83 (br d, J=7.5 Hz, 1H), 7.07-7.15 (m, 1H), 7.18-7.29 (m, 4H), 7.41 (ddd, J=8.2, 1.9, 0.9 Hz, 1H), 7.47-7.55 (m, 1H), 7.63-7.72 (m, 2H), 8.41-8.49 (m, 2H); LCMS (method A): Rt 1.84 min

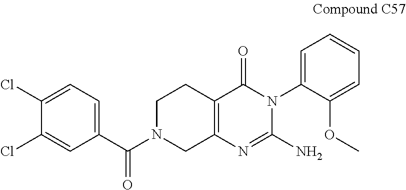 [SMILES: COc1ccccc1-n1c(N)nc2c(c1=O)CCN(C(=O)c1ccc(Cl)c(Cl)c1)C2]

¹H NMR (400 MHz, DMSO-d₆, 80° C.) δ ppm 2.39 (t, J=5.8 Hz, 2H), 3.51-3.71 (m, 2H), 3.75 (s, 3H), 4.11-4.41 (m, 2H), 6.01 (br s, 2H), 7.07 (td, J=7.5, 1.1 Hz, 1H), 7.14 (dd, J=7.7, 1.4 Hz, 1H), 7.20 (dd, J=8.4, 0.9 Hz, 1H), 7.40-7.49 (m, 2H), 7.66-7.72 (m, 2H); LCMS (method A): Rt 1.61 min

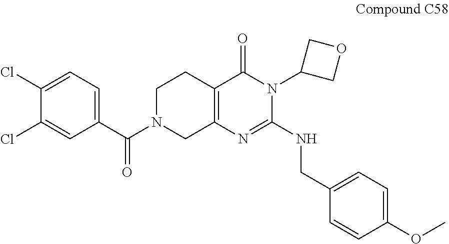 [SMILES: COc1ccc(CNc2nc3c(c(=O)n2C2COC2)CCN(C(=O)c2ccc(Cl)c(Cl)c2)C3)cc1]

¹H NMR (400 MHz, CDCl₃, 57° C.) δ ppm 2.58 (br t, J=5.3 Hz, 2H), 3.56-3.65 (m, 1H), 3.68 (br s, 2H), 3.74-3.78 (m, 1H), 3.79 (s, 3H), 3.86 (ddt, J=10.2, 6.8, 3.4, 3.4 Hz, 1H), 3.94-4.03 (m, 1H), 4.03-4.15 (m, 1H), 4.27-4.56 (m, 3H), 4.73 (br d, J=15.2 Hz, 1H), 6.84-6.89 (m, 2H), 7.22 (br d, J=8.4 Hz, 2H), 7.27 (dd, J=8.1, 2.0 Hz, 1H), 7.50 (d, J=8.1 Hz, 1H), 7.55 (d, J=2.0 Hz, 1H); LCMS (method C): Rt 0.95 min

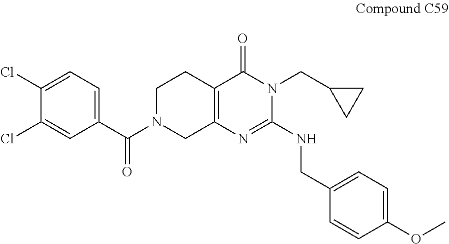 [SMILES: COc1ccc(CNc2nc3c(c(=O)n2CC2CC2)CCN(C(=O)c2ccc(Cl)c(Cl)c2)C3)cc1]

¹H NMR (400 MHz, CDCl₃, 57° C.) δ ppm 0.36-0.45 (m, 2H), 0.49-0.59 (m, 2H), 0.93-1.04 (m, 1H), 2.58 (br t, J=5.5 Hz, 2H), 3.50-3.78 (m, 2H), 3.80 (s, 3H), 3.87 (d, J=6.4 Hz, 2H), 4.40 (br s, 2H), 4.53 (br d, J=4.6 Hz, 2H), 4.92 (br t, J=4.7 Hz, 1H), 6.85-6.91 (m, 2H), 7.22-7.25 (m, 1H), 7.27 (dd, J=8.1, 2.0 Hz, 2H), 7.49 (d, J=8.4 Hz, 1H), 7.55 (d, J=1.8 Hz, 1H); LCMS (method C): Rt 1.17 min

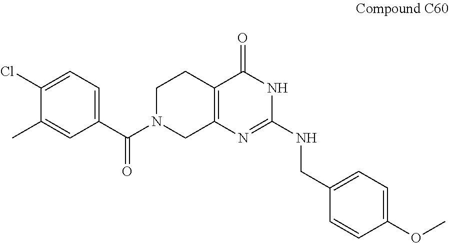

¹H NMR (400 MHz, DMSO-d₆, 80° C.) δ ppm 2.33-2.33 (m, 2H), 2.37 (s, 3H), 3.58 (br s, 2H), 3.73 (s, 3H), 4.22 (br s, 2H), 4.35 (br d, J=5.3 Hz, 2H), 6.51 (br t, J=5.2 Hz, 1H), 6.84-6.91 (m, 2H), 7.20-7.27 (m, 3H), 7.38 (d, J=1.8 Hz, 1H), 7.46 (d, J=8.1 Hz, 1H), 10.47 (br s, 1H); LCMS (method B): Rt 1.81 min

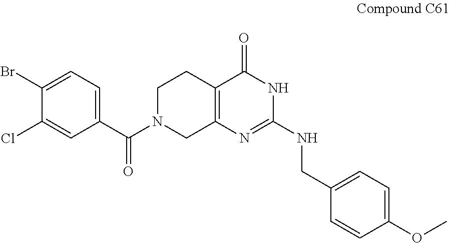 [SMILES: COc1ccc(CNc2nc3c(c(=O)[nH]2)CCN(C(=O)c2ccc(Br)c(Cl)c2)C3)cc1]

¹H NMR (400 MHz, DMSO-d₆, 80° C.) δ ppm 2.36 (t, J=5.8 Hz, 2H), 3.58 (br s, 2H), 3.73 (s, 3H), 4.22 (br s, 2H), 4.36 (br d, J=5.3 Hz, 2H), 6.51 (br t, J=5.1 Hz, 1H), 6.85-6.90 (m, 2H), 7.23 (d, J=8.4 Hz, 2H), 7.31 (dd, J=8.1, 2.0 Hz, 1H), 7.65 (d, J=1.8 Hz, 1H), 7.82 (d, J=8.1 Hz, 1H), 10.48 (br s, 1H); LCMS (method A): Rt 1.83 min

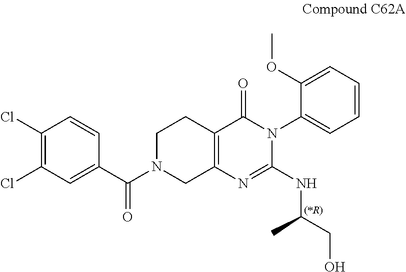 [SMILES: COc1ccccc1-n1c(N[C@H](C)CO)nc2c(c1=O)CCN(C(=O)c1ccc(Cl)c(Cl)c1)C2]

¹H NMR (400 MHz, DMSO-d₆, 80° C.) δ ppm 0.94-1.09 (m, 3H), 2.40 (t, J=5.8 Hz, 2H), 3.22-3.37 (m, 2H), 3.54-3.73 (m, 2H), 3.75 (d, J=2.2 Hz, 3H), 3.91-4.06 (m, 1H), 4.17-4.47 (m, 3H), 4.90 (dd, J=14.2, 7.8 Hz, 1H), 7.07-7.14 (m, 1H), 7.14-7.21 (m, 1H), 7.23 (dt, J=8.4, 1.3 Hz, 1H), 7.45 (dd, J=8.3, 1.9 Hz, 1H), 7.46-7.53 (m, 1H), 7.67-7.74 (m, 2H); LCMS (method D): Rt 1.72 min

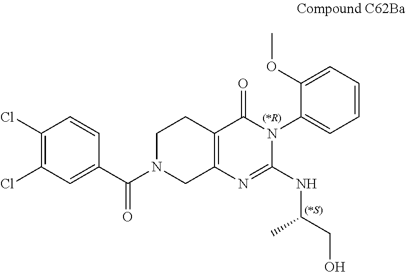 [SMILES: COc1ccccc1-n1c(N[C@@H](C)CO)nc2c(c1=O)CCN(C(=O)c1ccc(Cl)c(Cl)c1)C2]

¹H NMR (400 MHz, DMSO-d₆, 80° C.) δ ppm 1.00 (d, J=6.6 Hz, 3H), 2.40 (t, J=5.8 Hz, 2H), 3.24-3.36 (m, 2H), 3.49-3.74 (m, 2H), 3.76 (s, 3H), 3.94-4.08 (m, 1H), 4.19-4.39 (m, 2H), 4.42 (br t, J=4.4 Hz, 1H), 4.87 (br d, J=7.9 Hz, 1H), 7.07-7.13 (m, 1H), 7.18 (dd, J=7.7, 1.8 Hz, 1H), 7.24 (dd, J=8.5, 1.0 Hz, 1H), 7.45 (dd, J=8.3, 1.9 Hz, 1H), 7.47-7.52 (m, 1H), 7.66-7.76 (m, 2H); LCMS (method D): Rt 1.72 min

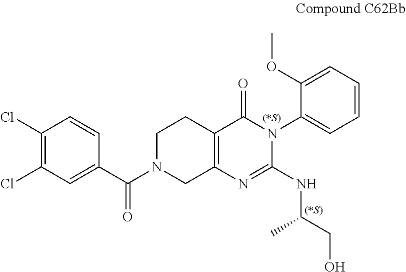 [SMILES: COc1ccccc1-n1c(N[C@@H](C)CO)nc2c(c1=O)CCN(C(=O)c1ccc(Cl)c(Cl)c1)C2]

¹H NMR (400 MHz, DMSO-d₆, 80° C.) δ ppm 1.03 (d, J=6.6 Hz, 3H), 2.40 (t, J=5.8 Hz, 2H), 3.16-3.37 (m, 2H), 3.52-3.74 (m, 2H), 3.76 (s, 3H), 3.91-4.04 (m, 1H), 4.21-4.37 (m, 2H), 4.41 (br t, J=4.4 Hz, 1H), 4.90 (br d, J=7.9 Hz, 1H), 7.06-7.12 (m, 1H), 7.16 (dd, J=7.7, 1.8 Hz, 1H), 7.23 (dd, J=8.4, 1.2 Hz, 1H), 7.45 (dd, J=8.1, 2.0 Hz, 1H), 7.46-7.52 (m, 1H), 7.67-7.73 (m, 2H); LCMS (method D): Rt 1.74 min

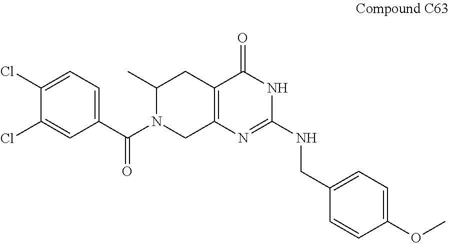 [SMILES: COc1ccc(CNc2nc3c(c(=O)[nH]2)CC(C)N(C(=O)c2ccc(Cl)c(Cl)c2)C3)cc1]

¹H NMR (400 MHz, DMSO-d₆, 80° C.) δ ppm 1.12 (d, J=6.8 Hz, 3H), 2.31 (br d, J=16.3 Hz, 1H), 2.40-2.47 (m, 1H), 3.73 (s, 3H), 3.82-4.02 (m, 1H), 4.11-4.73 (m, 4H), 6.63 (br s, 1H), 6.84-6.90 (m, 2H), 7.20-7.28 (m, 2H), 7.38 (dd, J=8.1, 2.0 Hz, 1H), 7.65 (d, J=1.8 Hz, 1H), 7.68 (d, J=8.1 Hz, 1H), 9.60-11.70 (m, 1H); LCMS (method B): Rt 1.91 min

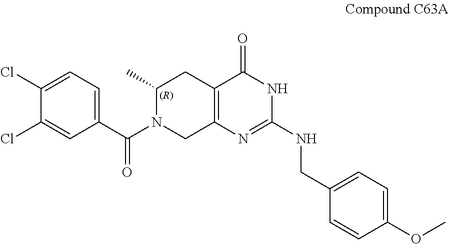

¹H NMR (400 MHz, DMSO-d₆, 81° C.) δ ppm 1.12 (d, J=6.8 Hz, 3H), 2.31 (br d, J=16.3 Hz, 1H), 2.42-2.51 (m, 1H), 3.74 (s, 3H), 3.85-4.03 (m, 1H), 4.29-4.58 (m, 4H), 6.52 (br t, J=5.4 Hz, 1H), 6.84-6.91 (m, 2H), 7.20-7.28 (m, 2H), 7.39 (dd, J=8.1, 2.0 Hz, 1H), 7.65 (d, J=2.0 Hz, 1H), 7.68 (d, J=8.1 Hz, 1H), 10.50 (br s, 1H); LCMS (method D): Rt 1.85 min

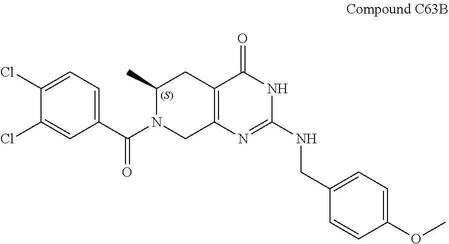

¹H NMR (400 MHz, DMSO-d₆, 81° C.) δ ppm 1.12 (d, J=6.8 Hz, 3H), 2.31 (br d, J=16.3 Hz, 1H), 2.41-2.52 (m, 1H), 3.74 (s, 3H), 3.82-4.03 (m, 1H), 4.17-4.87 (m, 4H), 6.55 (br t, J=4.5 Hz, 1H), 6.85-6.91 (m, 2H), 7.20-7.28 (m, 2H), 7.39 (dd, J=8.1, 2.0 Hz, 1H), 7.65 (d, J=2.0 Hz, 1H), 7.68 (d, J=8.1 Hz, 1H), 10.50 (br s, 1H); LCMS (method D): Rt 1.85 min

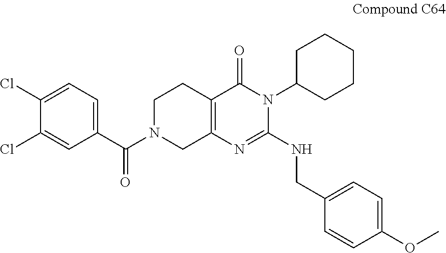 [SMILES: COc1ccc(CNc2nc3c(c(=O)n2C2CCCCC2)CCN(C(=O)c2ccc(Cl)c(Cl)c2)C3)cc1]

¹H NMR (600 MHz, DMSO-d₆, 118° C.) δ ppm 7.64 (d, J=8.3 Hz, 1H), 7.62 (d, J=1.8 Hz, 1H), 7.37 (dd, J=8.2, 1.9 Hz, 1H), 7.21-7.25 (m, 2H), 7.00 (br t, J=5.2 Hz, 1H), 6.82-6.86 (m, 2H), 4.46 (d, J=5.6 Hz, 2H), 4.33-4.40 (m, 1H), 4.17 (s, 2H), 3.73 (s, 3H), 3.56 (br t, J=5.5 Hz, 2H), 2.45-2.54 (m, 2H), 2.36 (t, J=5.9 Hz, 2H), 1.78 (br d, J=13.4 Hz, 2H), 1.58-1.65 (m, 3H), 1.35-1.44 (m, 2H), 1.18-1.28 (m, 1H); LCMS (method C): Rt 1.27 min

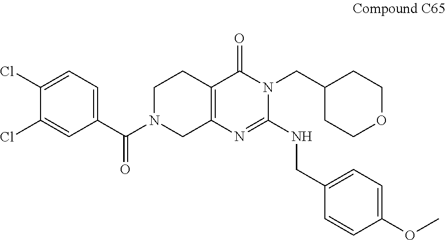 [SMILES: COc1ccc(CNc2nc3c(c(=O)n2CC2CCOCC2)CCN(C(=O)c2ccc(Cl)c(Cl)c2)C3)cc1]

¹H NMR (400 MHz, CDCl₃, 57° C.) δ ppm 1.34-1.47 (m, 2H), 1.47-1.52 (m, 2H), 1.92-2.04 (m, 1H), 2.58 (br t, J=5.5 Hz, 2H), 3.29 (td, J=11.7, 2.2 Hz, 2H), 3.49-3.74 (m, 2H), 3.75-3.85 (m, 1H), 3.76-3.83 (m, 2H), 3.80 (s, 2H), 3.88-3.96 (m, 2H), 4.40 (br s, 2H), 4.52 (br d, J=4.8 Hz, 2H), 4.69 (br t, J=4.4 Hz, 1H), 6.88 (m, 2H), 7.23 (d, J=8.4 Hz, 2H), 7.27 (dd, J=8.1, 2.0 Hz, 1H), 7.50 (d, J=8.4 Hz, 1H), 7.54 (d, J=2.0 Hz, 1H); LCMS (method C): Rt 1.09 min

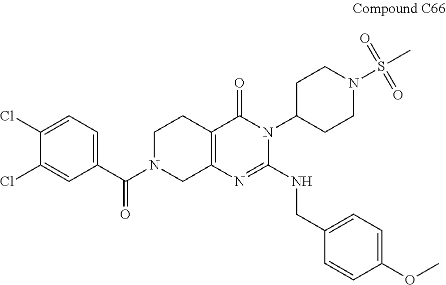 [SMILES: COc1ccc(CNc2nc3c(c(=O)n2C2CCN(S(C)(=O)=O)CC2)CCN(C(=O)c2ccc(Cl)c(Cl)c2)C3)cc1]

¹H NMR (400 MHz, DMSO-d₆, 100° C.) δ ppm 1.62-1.69 (m, 2H), 2.35 (t, J=5.8 Hz, 2H), 2.76-2.91 (m, 7H), 3.51-3.60 (m, 2H), 3.69-3.76 (m, 5H), 4.17 (s, 2H), 4.33-4.42 (m, 1H), 4.45 (d, J=4.8 Hz, 2H), 6.82-6.87 (m, 2H), 7.23 (d, J=8.6 Hz, 2H), 7.29 (br s, 1H), 7.38 (dd, J=8.2, 1.9 Hz, 1H), 7.63 (d, J=2.0 Hz, 1H), 7.66 (d, J=8.3 Hz, 1H); LCMS (method D): Rt 1.98 min

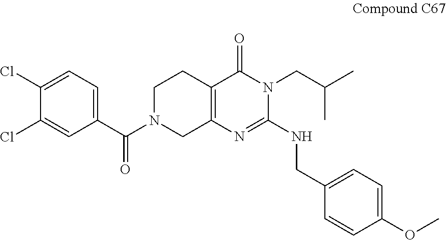 [SMILES: COc1ccc(CNc2nc3c(c(=O)n2CC(C)C)CCN(C(=O)c2ccc(Cl)c(Cl)c2)C3)cc1]

¹H NMR (400 MHz, CDCl₃, 57° C.) δ ppm 0.93 (d, J=6.6 Hz, 6H), 2.07 (spt, J=6.8 Hz, 1H), 2.57 (br t, J=5.4 Hz, 2H), 3.55-3.72 (m, 2H), 3.74 (d, J=7.7 Hz, 2H), 3.79 (s, 3H), 4.19-4.47 (m, 2H), 4.51 (br d, J=4.6 Hz, 2H), 4.80 (br t, J=4.3 Hz, 1H), 6.86 (d, J=8.6 Hz, 2H), 7.22 (br d, J=8.4 Hz, 2H), 7.26 (dd, J=8.1, 2.0 Hz, 1H), 7.49 (d, J=8.4 Hz, 1H), 7.54 (d, J=2.0 Hz, 1H); LCMS (method C): Rt 1.18 min

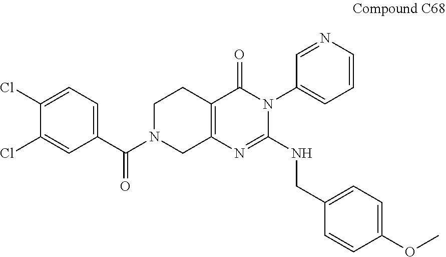 [SMILES: COc1ccc(CNc2nc3c(c(=O)n2-c2cccnc2)CCN(C(=O)c2ccc(Cl)c(Cl)c2)C3)cc1]

¹H NMR (400 MHz, DMSO-d₆, 100° C.) δ ppm 2.41 (t, J=5.8 Hz, 2H), 3.58-3.67 (m, 2H), 3.72 (s, 3H), 4.29 (s, 2H), 4.32-4.36 (m, 2H), 6.42 (br s, 1H), 6.78-6.83 (m, 2H), 7.15 (d, J=8.6 Hz, 2H), 7.41 (dd, J=8.1, 2.0 Hz, 1H), 7.56 (dd, J=8.0, 4.7 Hz, 1H), 7.65-7.69 (m, 2H), 7.71 (ddd, J=8.0, 2.4, 1.6 Hz, 1H), 8.44 (d, J=2.4 Hz, 1H), 8.66 (dd, J=4.8, 1.5 Hz, 1H); LCMS (method C): Rt 1.04 min

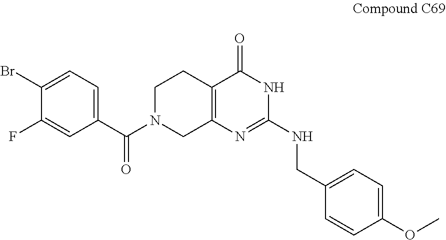 [SMILES: COc1ccc(CNc2nc3c(c(=O)[nH]2)CCN(C(=O)c2ccc(Br)c(F)c2)C3)cc1]

¹H NMR (400 MHz, DMSO-d₆, 125° C.) δ ppm 2.37 (t, J=5.8 Hz, 2H), 3.59 (t, J=6.0 Hz, 2H), 3.74 (s, 3H), 4.22 (s, 2H), 4.36 (br d, J=4.6 Hz, 2H), 6.39 (br s, 1H), 6.86 (d, J=8.7 Hz, 2H), 7.16-7.25 (m, 3H), 7.36 (dd, J=9.0, 1.8 Hz, 1H), 7.73 (dd, J=7.9, 7.2 Hz, 1H), 10.27 (br s, 1H); LCMS (method B): Rt 1.77 min

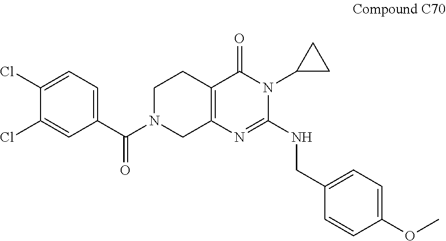 [SMILES: COc1ccc(CNc2nc3c(c(=O)n2C2CC2)CCN(C(=O)c2ccc(Cl)c(Cl)c2)C3)cc1]

¹H NMR (400 MHz, DMSO-d₆, 100° C.) δ ppm 0.69-0.76 (m, 2H), 1.14-1.22 (m, 2H), 2.36 (t, J=5.8 Hz, 2H), 2.55-2.65 (m, 1H), 3.56 (br t, J=5.3 Hz, 2H), 3.73 (s, 3H), 4.18 (s, 2H), 4.47 (d, J=5.7 Hz, 2H), 6.82-6.89 (m, 2H), 7.01 (br t, J=5.3 Hz, 1H), 7.23-7.29 (m, 2H), 7.38 (dd, J=8.3, 1.9 Hz, 1H), 7.63 (d, J=2.0 Hz, 1H), 7.66 (d, J=8.1 Hz, 1H); LCMS (method C): Rt 1.10 min

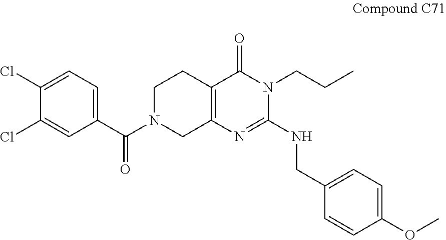 [SMILES: CCCn1c(NCc2ccc(OC)cc2)nc2c(c1=O)CCN(C(=O)c1ccc(Cl)c(Cl)c1)C2]

¹H NMR (400 MHz, DMSO-d₆, 100° C.) δ ppm 0.88 (t, J=7.4 Hz, 3H), 1.53-1.64 (m, 2H), 2.39 (t, J=5.8 Hz, 2H), 3.50-3.68 (m, 2H), 3.73 (s, 3H), 3.89 (t, J=3.9 Hz, 2H), 4.20 (s, 2H), 4.40-4.50 (m, 2H), 6.81-6.87 (m, 2H), 7.17-7.28 (m, 3H), 7.38 (dd, J=8.3, 1.9 Hz, 1H), 7.63 (d, J=2.0 Hz, 1H), 7.66 (d, J=7.8 Hz, 1H); LCMS (method C): Rt1.16 min

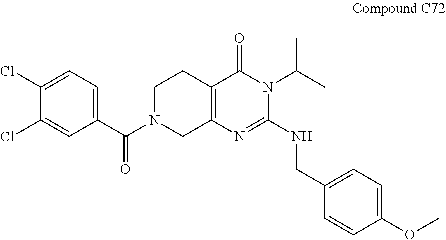 [SMILES: COc1ccc(CNc2nc3c(c(=O)n2C(C)C)CCN(C(=O)c2ccc(Cl)c(Cl)c2)C3)cc1]

¹H NMR (400 MHz, DMSO-d₆, 100° C.) δ ppm 1.46 (d, J=6.8 Hz, 6H), 2.35 (t, J=5.8 Hz, 2H), 3.50-3.62 (m, 2H), 3.73 (s, 3H), 4.17 (s, 2H), 4.39-4.49 (m, 2H), 4.84 (spt, J=6.8 Hz, 1H), 6.81-6.86 (m, 2H), 6.99 (br s, 1H), 7.23 (br d, J=8.6 Hz, 2H), 7.38 (dd, J=8.1, 2.0 Hz, 1H), 7.63 (d, J=2.0 Hz, 1H), 7.66 (d, J=8.4 Hz, 1H); LCMS (method C): Rt 1.17 min

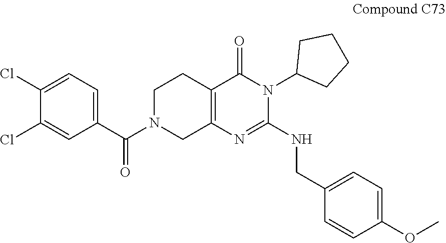 [SMILES: COc1ccc(CNc2nc3c(c(=O)n2C2CCCC2)CCN(C(=O)c2ccc(Cl)c(Cl)c2)C3)cc1]

¹H NMR (400 MHz, CDCl₃, 56° C.) δ ppm 1.60-1.84 (m, 4H), 1.91-2.05 (m, 4H), 2.57 (br t, J=5.3 Hz, 2H), 3.52-3.77 (m, 2H), 3.80 (s, 3H), 4.21-4.46 (m, 2H), 4.46-4.54 (m, 2H), 4.54-4.66 (m, 1H), 5.39 (quin, J=9.1 Hz, 1H), 6.86-6.91 (m, 2H), 7.20-7.25 (m, 2H), 7.27 (dd, J=8.1, 2.0 Hz, 1H), 7.50 (d, J=8.4 Hz, 1H), 7.55 (d, J=1.8 Hz, 1H); LCMS (method C): Rt 1.25 min

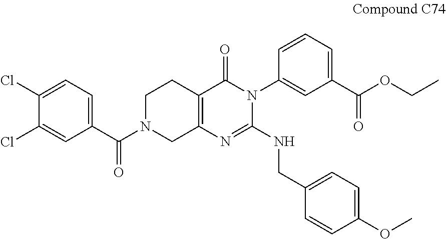 [SMILES: CCOC(=O)c1cccc(-n2c(NCc3ccc(OC)cc3)nc3c(c2=O)CCN(C(=O)c2ccc(Cl)c(Cl)c2)C3)c1]

¹H NMR (400 MHz, DMSO-d₆, 100° C.) δ ppm 1.33 (t, J=7.2 Hz, 3H), 2.41 (br t, J=5.7 Hz, 2H), 3.57-3.68 (m, 2H), 3.71 (br s, 3H), 4.24-4.41 (m, 6H), 6.25 (br t, J=5.1 Hz, 1H), 6.80 (br d, J=8.6 Hz, 2H), 7.14 (br d, J=8.6 Hz, 2H), 7.42 (dd, J=8.1, 2.0 Hz, 1H), 7.47-7.55 (m, 1H), 7.64-7.73 (m, 3H), 7.77 (t, J=1.5 Hz, 1H), 8.06 (d, J=7.9 Hz, 1H); LCMS (method C): Rt 1.22 min

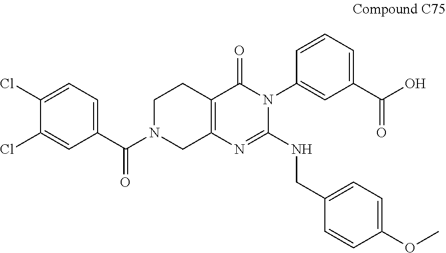 [SMILES: COc1ccc(CNc2nc3c(c(=O)n2-c2cccc(C(=O)O)c2)CCN(C(=O)c2ccc(Cl)c(Cl)c2)C3)cc1]

¹H NMR (400 MHz, DMSO-d₆, 100° C.) δ ppm 2.41 (br t, J=5.7 Hz, 2H), 3.05-3.19 (m, 1H), 3.59-3.69 (m, 2H), 3.71 (s, 3H), 4.26-4.43 (m, 4H), 5.91-6.00 (m, 1H), 6.80 (br d, J=8.6 Hz, 2H), 7.13 (br d, J=8.6 Hz, 2H), 7.22 (br d, J=7.9 Hz, 1H), 7.42 (dd, J=8.3, 1.9 Hz, 1H), 7.50 (t, J=7.7 Hz, 1H), 7.66-7.80 (m, 3H), 8.00 (d, J=7.7 Hz, 1H); LCMS (method C): Rt 0.85 min

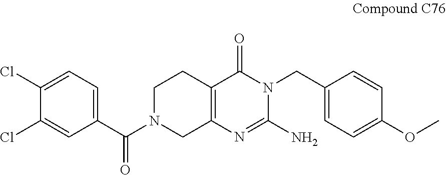 [SMILES: COc1ccc(Cn2c(N)nc3c(c2=O)CCN(C(=O)c2ccc(Cl)c(Cl)c2)C3)cc1]

¹H NMR (400 MHz, DMSO-d₆, 125° C.) δ ppm 2.43 (t, J=5.8 Hz, 2H), 3.60 (t, J=5.8 Hz, 2H), 3.73 (s, 3H), 4.21 (s, 2H), 5.08 (s, 2H), 6.40 (br s, 2H), 6.83-6.88 (m, 2H), 7.18-7.23 (m, 2H), 7.38 (dd, J=8.1, 2.0 Hz, 1H), 7.62 (d, J=2.0 Hz, 1H), 7.65 (d, J=8.4 Hz, 1H); LCMS (method B): Rt 1.79 min

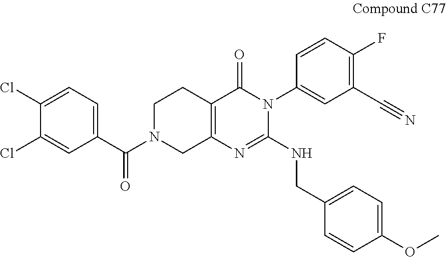 [SMILES: COc1ccc(CNc2nc3c(c(=O)n2-c2ccc(F)c(C#N)c2)CCN(C(=O)c2ccc(Cl)c(Cl)c2)C3)cc1]

¹H NMR (400 MHz, DMSO-d₆, 100° C.) δ ppm 2.41 (t, J=5.8 Hz, 2H), 3.62 (br t, J=5.0 Hz, 2H), 3.72 (s, 3H), 4.28 (s, 2H), 4.35 (br s, 2H), 6.48 (br s, 1H), 6.81 (d, J=8.6 Hz, 2H), 7.16 (d, J=8.6 Hz, 2H), 7.40 (dd, J=8.1, 2.0 Hz, 1H), 7.61-7.73 (m, 4H), 7.92 (dd, J=5.8, 2.5 Hz, 1H); LCMS (method C): Rt 1.15 min

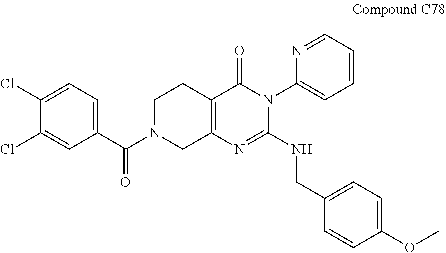 [SMILES: COc1ccc(CNc2nc3c(c(=O)n2-c2ccccn2)CCN(C(=O)c2ccc(Cl)c(Cl)c2)C3)cc1]

¹H NMR (400 MHz, DMSO-d₆, 100° C.) δ ppm 2.41 (t, J=5.9 Hz, 2H), 3.62 (br t, J=4.7 Hz, 2H), 3.71 (s, 3H), 4.28 (s, 2H), 4.35 (d, J=5.3 Hz, 2H), 6.26-6.35 (m, 1H), 6.77-6.84 (m, 2H), 7.15 (d, J=8.6 Hz, 2H), 7.39-7.44 (m, 2H), 7.51 (ddd, J=7.5, 4.8, 1.1 Hz, 1H), 7.65-7.69 (m, 2H), 8.00 (td, J=7.7, 1.9 Hz, 1H), 8.63 (ddd, J=4.8, 1.9, 0.8 Hz, 1H); LCMS (method C): Rt 1.06 min

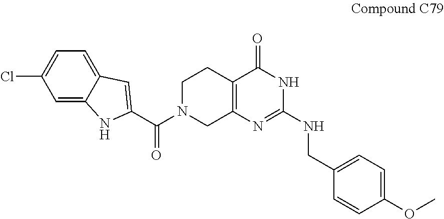 [SMILES: COc1ccc(CNc2nc3c(c(=O)[nH]2)CCN(C(=O)c2cc4ccc(Cl)cc4[nH]2)C3)cc1]

¹H NMR (400 MHz, DMSO-d₆, 81° C.) δ ppm 2.43-2.47 (m, 2H), 3.73 (s, 3H), 3.88 (t, J=5.8 Hz, 2H), 4.37 (d, J=5.3 Hz, 2H), 4.47 (s, 2H), 6.52 (br t, J=4.9 Hz, 1H), 6.85-6.94 (m, 3H), 7.05 (dd, J=8.6, 2.0 Hz, 1H), 7.24 (d, J=8.6 Hz, 2H), 7.43-7.47 (m, 1H), 7.63 (d, J=8.6 Hz, 1H), 10.50 (br s, 1H), 11.50 (br s, 1H); LCMS (method C): Rt 0.99 min

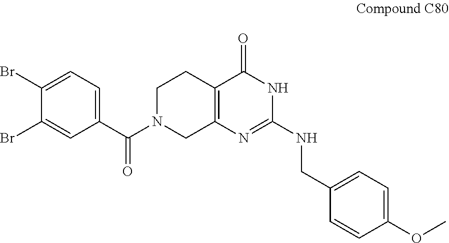 [SMILES: COc1ccc(CNc2nc3c(c(=O)[nH]2)CCN(C(=O)c2ccc(Br)c(Br)c2)C3)cc1]

¹H NMR (400 MHz, DMSO-d₆, 81° C.) δ ppm 2.36 (t, J=5.8 Hz, 2H), 3.49-3.67 (m, 2H), 3.73 (s, 3H), 4.22 (br s, 2H), 4.36 (d, J=5.0 Hz, 2H), 6.51 (br t, J=4.7 Hz, 1H), 6.84-6.91 (m, 2H), 7.23 (d, J=8.6 Hz, 2H), 7.35 (dd, J=8.1, 2.0 Hz, 1H), 7.77 (d, J=1.8 Hz, 1H), 7.81 (d, J=8.3 Hz, 1H), 10.48 (br s, 1H); LCMS (method B): Rt 1.88 min

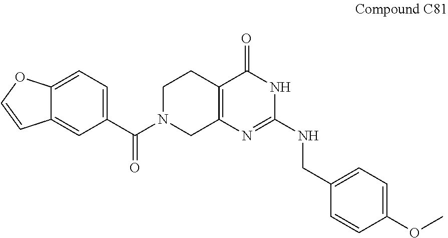 [SMILES: COc1ccc(CNc2nc3c(c(=O)[nH]2)CCN(C(=O)c2ccc4occc4c2)C3)cc1]

¹H NMR (400 MHz, DMSO-d₆, 81° C.) δ ppm 2.37 (t, J=5.7 Hz, 2H), 3.62 (br t, J=5.0 Hz, 2H), 3.73 (s, 3H), 4.27 (s, 2H), 4.34 (d, J=5.5 Hz, 2H), 6.50 (br t, J=5.2 Hz, 1H), 6.85-6.91 (m, 2H), 6.99 (dd, J=2.2, 0.9 Hz, 1H), 7.22 (d, J=8.8 Hz, 2H), 7.36 (dd, J=8.4, 1.6 Hz, 1H), 7.63 (d, J=8.6 Hz, 1H), 7.73 (d, J=1.3 Hz, 1H), 8.00 (d, J=2.2 Hz, 1H), 10.46 (br s, 1H); LCMS (method B): Rt 1.62 min

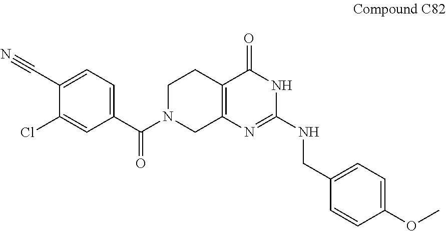 [SMILES: COc1ccc(CNc2nc3c(c(=O)[nH]2)CCN(C(=O)c2ccc(C#N)c(Cl)c2)C3)cc1]

¹H NMR (400 MHz, DMSO-d₆, 101° C.) δ ppm 2.37 (t, J=5.8 Hz, 2H), 3.49-3.63 (m, 2H), 3.74 (s, 3H), 4.22 (br s, 2H), 4.36 (br d, J=4.8 Hz, 2H), 6.47 (br s, 1H), 6.85-6.89 (m, 2H), 7.22 (d, J=8.6 Hz, 2H), 7.55 (dd, J=8.0, 1.4 Hz, 1H), 7.74 (d, J=1.3 Hz, 1H), 7.98 (d, J=7.9 Hz, 1H), 10.39 (br s, 1H); LCMS (method B): Rt 1.66 min

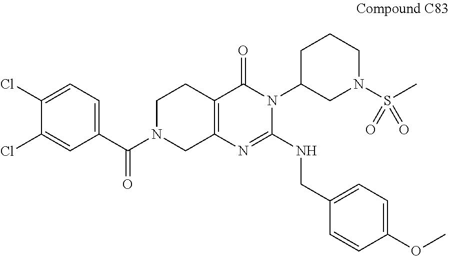 [SMILES: COc1ccc(CNc2nc3c(c(=O)n2C2CCCN(S(C)(=O)=O)C2)CCN(C(=O)c2ccc(Cl)c(Cl)c2)C3)cc1]

¹H NMR (400 MHz, CDCl₃, 56° C.) δ ppm 1.64-1.94 (m, 4H), 2.50 (br t, J=5.1 Hz, 2H), 2.72-2.81 (m, 1H), 2.80 (s, 3H), 2.82-2.94 (m, 1H), 3.52-3.75 (m, 3H), 3.75-3.81 (m, 4H), 3.90 (t, J=11.3 Hz, 1H), 4.25-4.45 (m, 2H), 4.49 (br d, J=4.8 Hz, 2H), 5.47-5.59 (m, 1H), 6.82-6.89 (m, 2H), 7.21-7.30 (m, 3H), 7.50 (d, J=8.1 Hz, 1H), 7.54 (d, J=2.0 Hz, 1H); LCMS (method C): Rt 1.14 min

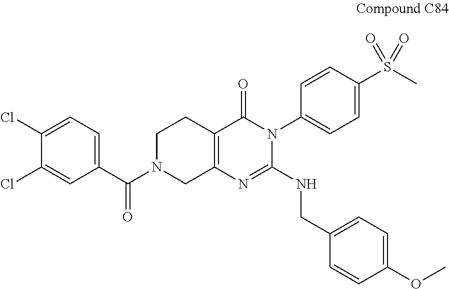 [SMILES: COc1ccc(CNc2nc3c(c(=O)n2-c2ccc(S(C)(=O)=O)cc2)CCN(C(=O)c2ccc(Cl)c(Cl)c2)C3)cc1]

¹H NMR (400 MHz, DMSO-d₆, 100° C.) δ ppm 2.41 (t, J=5.7 Hz, 2H), 3.24 (s, 3H), 3.62 (br t, J=4.4 Hz, 2H), 3.72 (s, 3H), 4.28 (br s, 2H), 4.35 (d, J=5.9 Hz, 2H), 6.36 (t, J=5.7 Hz, 1H), 6.79-6.84 (m, 2H), 7.15 (br d, J=8.6 Hz, 2H), 7.41 (dd, J=8.4, 2.0 Hz, 1H), 7.52-7.57 (m, 2H), 7.63-7.71 (m, 2H), 8.05-8.10 (m, 2H); LCMS (method C): Rt 1.09 min

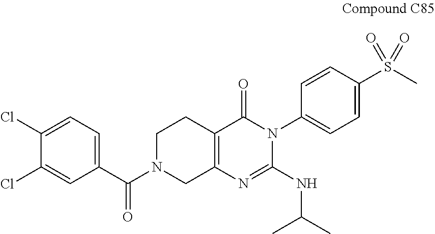 [SMILES: CC(C)Nc1nc2c(c(=O)n1-c1ccc(S(C)(=O)=O)cc1)CCN(C(=O)c1ccc(Cl)c(Cl)c1)C2]

¹H NMR (400 MHz, DMSO-d₆, 100° C.) δ ppm 1.07 (d, J=6.6 Hz, 6H), 2.41 (t, J=5.7 Hz, 2H), 3.25 (s, 3H), 3.64 (br t, J=4.7 Hz, 2H), 4.15 (dq, J=14.0, 6.8 Hz, 1H), 4.31 (s, 2H), 5.24 (br d, J=7.0 Hz, 1H), 7.43 (dd, J=8.4, 2.0 Hz, 1H), 7.50-7.55 (m, 2H), 7.66-7.70 (m, 2H), 8.05-8.10 (m, 2H); LCMS (method C): Rt 1.05 min

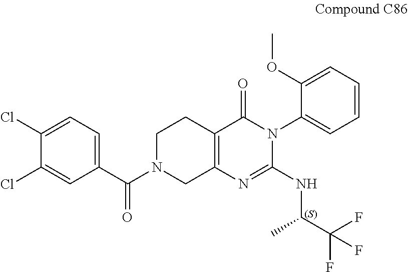

¹H NMR (400 MHz, DMSO-d₆, 81° C.) δ ppm 1.22 (d, J=7.0 Hz, 3H), 2.41 (t, J=5.8 Hz, 2H), 3.53-3.70 (m, 2H), 3.74 (d, J=15.6 Hz, 3H), 4.24-4.40 (m, 2H), 4.91-5.10 (m, 1H), 5.58-5.78 (m, 1H), 7.06-7.27 (m, 3H), 7.45 (dd, J=8.4, 2.0 Hz, 1H), 7.47-7.55 (m, 1H), 7.67-7.75 (m, 2H); LCMS (method D): Rt 2.05 min

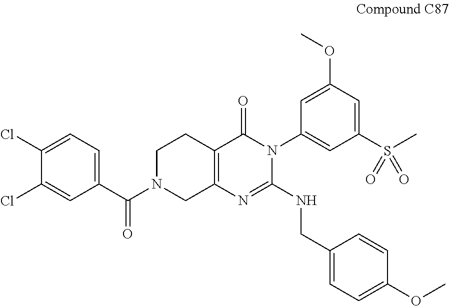 [SMILES: COc1ccc(CNc2nc3c(c(=O)n2-c2cc(OC)cc(S(C)(=O)=O)c2)CCN(C(=O)c2ccc(Cl)c(Cl)c2)C3)cc1]

¹H NMR (400 MHz, DMSO-d₆, 100° C.) δ ppm 7.68 (d, J=8.1 Hz, 1H), 7.66 (d, J=2.0 Hz, 1H), 7.53 (dd, J=2.4, 1.5 Hz, 1H), 7.41 (dd, J=8.3, 1.9 Hz, 1H), 7.36 (t, J=1.5 Hz, 1H), 7.23 (dd, J=2.4, 1.8 Hz, 1H), 7.16 (d, J=8.6 Hz, 2H), 6.78-6.84 (m, 2H), 6.42 (br t, J=5.3 Hz, 1H), 4.36 (d, J=5.7 Hz, 2H), 4.28 (s, 2H), 3.89 (s, 3H), 3.72 (s, 3H), 3.58-3.66 (m, 2H), 3.19 (s, 3H), 2.41 (t, J=6.8 Hz, 2H); LCMS (method C): Rt 1.10 min

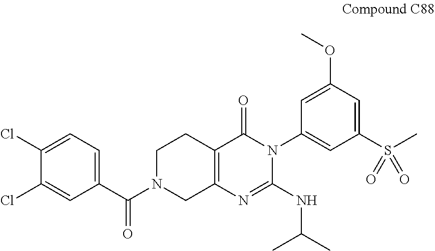 [SMILES: COc1cc(-n2c(NC(C)C)nc3c(c2=O)CCN(C(=O)c2ccc(Cl)c(Cl)c2)C3)cc(S(C)(=O)=O)c1]

¹H NMR (400 MHz, DMSO-d₆, 100° C.) δ ppm 1.07 (d, J=6.6 Hz, 6H), 2.41 (t, J=5.7 Hz, 2H), 3.20 (s, 3H), 3.53-3.73 (m, 2H), 3.90 (s, 3H), 4.11-4.22 (m, 1H), 4.31 (br s, 2H), 5.36 (br d, J=6.6 Hz, 1H), 7.17-7.24 (s, 1H), 7.32-7.38 (m, 1H), 7.42 (dd, J=8.3, 1.9 Hz, 1H), 7.49-7.56 (m, 1H), 7.63-7.75 (m, 2H); LCMS (method C): Rt 1.07 min

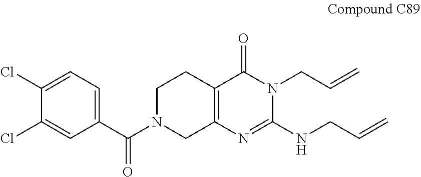 [SMILES: C=CCNc1nc2c(c(=O)n1CC=C)CCN(C(=O)c1ccc(Cl)c(Cl)c1)C2]

¹H NMR (400 MHz, DMSO-d₆, 125° C.) δ ppm 2.41 (t, J=5.9 Hz, 2H), 3.60 (t, J=5.9 Hz, 2H), 3.93 (tt, J=5.5, 1.6 Hz, 2H), 4.23 (s, 2H), 4.60 (dt, J=5.2, 1.7 Hz, 2H), 5.02-5.18 (m, 3H), 5.05 (dq, J=10.3, 1.5 Hz, 1H), 5.77-5.93 (m, 2H), 6.50-6.62 (m, 1H), 7.39 (dd, J=8.3, 1.9 Hz, 1H), 7.62 (d, J=2.0 Hz, 1H), 7.65 (d, J=8.3 Hz, 1H); LCMS (method D): Rt 1.87 min

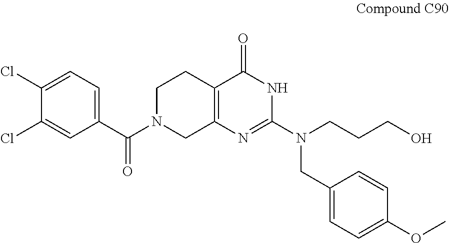 [SMILES: COc1ccc(CN(CCCO)c2nc3c(c(=O)[nH]2)CCN(C(=O)c2ccc(Cl)c(Cl)c2)C3)cc1]

¹H NMR (400 MHz, DMSO-d₆, 100° C.) δ ppm 1.66 (quin, J=6.6 Hz, 2H), 2.40 (t, J=5.8 Hz, 2H), 3.41 (t, J=6.1 Hz, 2H), 3.46 (t, J=7.0 Hz, 2H), 3.54-3.65 (m, 2H), 3.73 (s, 3H), 4.25 (s, 2H), 4.65 (s, 2H), 6.84-6.88 (m, 2H), 7.17 (br d, J=8.6 Hz, 2H), 7.40 (dd, J=8.1, 2.0 Hz, 1H), 7.65 (d, J=2.0 Hz, 1H), 7.67 (d, J=8.4 Hz, 1H); LCMS (method C): Rt 1.00 min

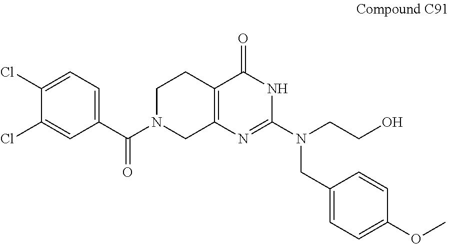 [SMILES: COc1ccc(CN(CCO)c2nc3c(c(=O)[nH]2)CCN(C(=O)c2ccc(Cl)c(Cl)c2)C3)cc1]

¹H NMR (400 MHz, DMSO-d₆, 100° C.) δ ppm 2.40 (t, J=5.8 Hz, 2H), 3.45-3.51 (m, 2H), 3.51-3.57 (m, 2H), 3.57-3.64 (m, 2H), 3.73 (s, 3H), 4.25 (s, 2H), 4.70 (s, 2H), 6.83-6.88 (m, 2H), 7.18 (br d, J=8.6 Hz, 2H), 7.40 (dd, J=8.4, 2.0 Hz, 1H), 7.65 (d, J=1.8 Hz, 1H), 7.67 (d, J=8.1 Hz, 1H); LCMS (method C): Rt 0.98 min

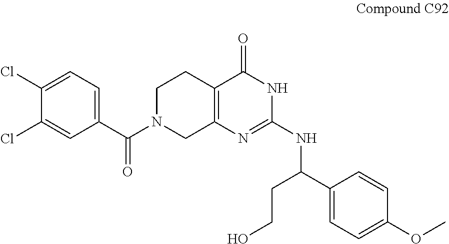 [SMILES: COc1ccc(C(CCO)Nc2nc3c(c(=O)[nH]2)CCN(C(=O)c2ccc(Cl)c(Cl)c2)C3)cc1]

¹H NMR (400 MHz, DMSO-d₆, 100° C.) δ ppm 1.83-1.96 (m, 2H), 2.33 (t, J=5.7 Hz, 2H), 3.36-3.45 (m, 2H), 3.48-3.60 (m, 2H), 3.73 (s, 3H), 4.09-4.25 (m, 2H), 4.99 (q, J=6.6 Hz, 1H), 6.59 (br d, J=7.5 Hz, 1H), 6.83-6.88 (m, 2H), 7.20-7.26 (m, 2H), 7.37 (dd, J=8.3, 1.9 Hz, 1H), 7.63 (d, J=2.0 Hz, 1H), 7.66 (d, J=8.1 Hz, 1H); LCMS (method C): Rt 0.88 min

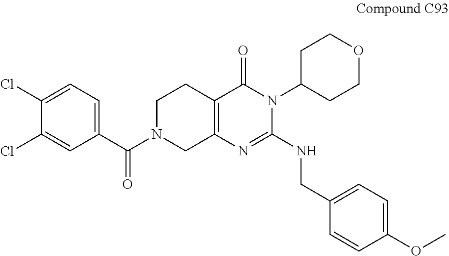 [SMILES: COc1ccc(CNc2nc3c(c(=O)n2C2CCOCC2)CCN(C(=O)c2ccc(Cl)c(Cl)c2)C3)cc1]

¹H NMR (400 MHz, DMSO-d₆, 100° C.) δ ppm 1.45-1.55 (m, 2H), 2.35 (t, J=5.9 Hz, 2H), 2.80 (qd, J=12.2, 4.7 Hz, 2H), 3.42 (td, J=11.9, 2.0 Hz, 2H), 3.49-3.65 (m, 2H), 3.73 (s, 3H), 3.93 (dd, J=11.2, 4.6 Hz, 2H), 4.17 (s, 2H), 4.45 (s, 2H), 4.47-4.55 (m, 1H), 6.80-6.88 (m, 2H), 7.20-7.26 (m, 2H), 7.29 (s, 1H), 7.38 (dd, J=8.4, 2.0 Hz, 1H), 7.63 (d, J=1.8 Hz, 1H), 7.66 (d, J=8.1 Hz, 1H); LCMS (method C): Rt 1.12 min

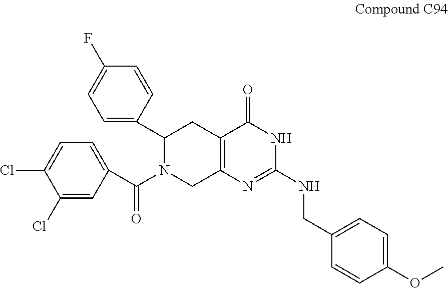 [SMILES: COc1ccc(CNc2nc3c(c(=O)[nH]2)CC(c2ccc(F)cc2)N(C(=O)c2ccc(Cl)c(Cl)c2)C3)cc1]

¹H NMR (400 MHz, DMSO-d₆, 81° C.) δ ppm 2.76 (br dd, J=16.6, 5.8 Hz, 1H), 3.01-3.14 (m, 1H), 3.69 (br d, J=18.5 Hz, 1H), 3.72 (s, 3H), 4.20-4.65 (m, 3H), 5.21-5.85 (m, 1H), 6.51 (br t, J=5.5 Hz, 1H), 6.81-6.90 (m, 2H), 7.06-7.28 (m, 6H), 7.44 (dd, J=8.4, 2.0 Hz, 1H), 7.68 (d, J=8.1 Hz, 1H), 7.71 (d, J=2.0 Hz, 1H), 10.49 (br s, 1H); LCMS (method B): Rt 2.10 min

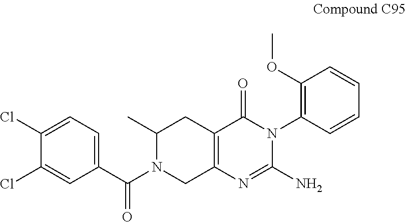 [SMILES: COc1ccccc1-n1c(N)nc2c(c1=O)CC(C)N(C(=O)c1ccc(Cl)c(Cl)c1)C2]

¹H NMR (400 MHz, DMSO-d₆, 80° C.) δ ppm 1.09-1.23 (m, 3H), 2.30 (br d, J=16.3 Hz, 1H), 2.49-2.60 (m, 1H), 3.75 (d, J=1.3 Hz, 3H), 3.88-4.05 (m, 1H), 4.13-5.00 (m, 2H), 6.01 (s, 2H), 7.04-7.10 (m, 1H), 7.12-7.23 (m, 2H), 7.37-7.50 (m, 2H), 7.65-7.72 (m, 2H); LCMS (method C): Rt 0.89 min

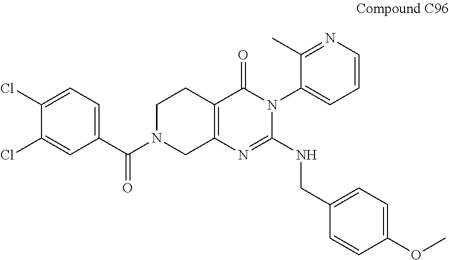 [SMILES: COc1ccc(CNc2nc3c(c(=O)n2-c2cccnc2C)CCN(C(=O)c2ccc(Cl)c(Cl)c2)C3)cc1]

¹H NMR (400 MHz, DMSO-d₆, 100° C.) δ ppm 2.18 (s, 3H), 2.37-2.45 (m, 2H), 3.62 (t, J=5.9 Hz, 2H), 3.72 (s, 3H), 4.26-4.41 (m, 4H), 6.44 (s, 1H), 6.79-6.83 (m, 2H), 7.15 (d, J=8.3 Hz, 2H), 7.37 (ddd, J=7.9, 4.8, 0.4 Hz, 1H), 7.42 (dd, J=8.1, 2.0 Hz, 1H), 7.58 (dd, J=7.9, 1.5 Hz, 1H), 7.67 (s, 1H), 7.68 (d, J=6.6 Hz, 1H), 8.55 (dd, J=4.7, 1.7 Hz, 1H); LCMS (method C): Rt 1.07 min

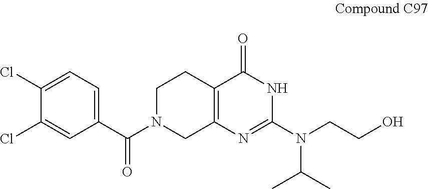 [SMILES: CC(C)N(CCO)c1nc2c(c(=O)[nH]1)CCN(C(=O)c1ccc(Cl)c(Cl)c1)C2]

¹H NMR (400 MHz, DMSO-d₆, 100° C.) δ ppm 1.11 (d, J=6.6 Hz, 6H), 2.38 (t, J=5.9 Hz, 2H), 3.36 (t, J=5.4 Hz, 2H), 3.54-3.65 (m, 6H), 4.23 (s, 2H), 4.64 (quin, J=6.7 Hz, 1H), 7.40 (dd, J=8.1, 2.0 Hz, 1H), 7.64 (d, J=2.0 Hz, 1H), 7.67 (d, J=8.1 Hz, 1H); LCMS (method C): Rt 0.93 min

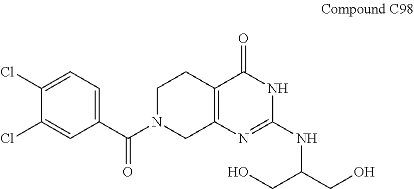 [SMILES: O=C(c1ccc(Cl)c(Cl)c1)N1CCc2c(nc(NC(CO)CO)[nH]c2=O)C1]

¹H NMR (400 MHz, DMSO-d₆, 100° C.) δ ppm 2.36 (t, J=5.8 Hz, 2H), 2.85-2.93 (m, 2H), 3.45-3.63 (m, 7H), 3.77-3.87 (m, 1H), 4.20 (s, 2H), 6.06-6.15 (m, 1H), 7.40 (dd, J=8.3, 1.9 Hz, 1H), 7.64 (d, J=2.0 Hz, 1H), 7.67 (d, J=8.1 Hz, 1H); LCMS (method C): Rt 0.69 min

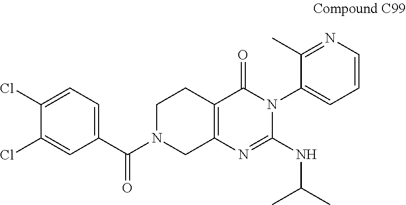 [SMILES: Cc1ncccc1-n1c(NC(C)C)nc2c(c1=O)CCN(C(=O)c1ccc(Cl)c(Cl)c1)C2]

¹H NMR (400 MHz, DMSO-d₆, 100° C.) δ ppm 1.02-1.09 (m, 6H), 2.20 (s, 3H), 2.36-2.45 (m, 2H), 3.56-3.71 (m, 2H), 4.17 (dq, J=13.9, 6.8 Hz, 1H), 4.24-4.38 (m, 2H), 5.36 (br d, J=6.6 Hz, 1H), 7.37 (dd, J=7.8, 4.7 Hz, 1H), 7.44 (dd, J=8.1, 2.0 Hz, 1H), 7.55 (dd, J=7.9, 1.5 Hz, 1H), 7.68 (s, 1H), 7.69 (d, J=6.6 Hz, 1H), 8.56 (dd, J=4.8, 1.5 Hz, 1H); LCMS (method C): Rt 1.03 min

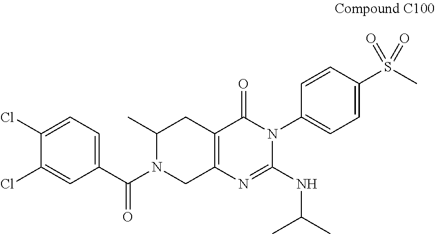 [SMILES: CC(C)Nc1nc2c(c(=O)n1-c1ccc(S(C)(=O)=O)cc1)CC(C)N(C(=O)c1ccc(Cl)c(Cl)c1)C2]

¹H NMR (400 MHz, DMSO-d₆, 126° C.) δ ppm 1.04-1.11 (m, 6H), 1.19 (d, J=6.8 Hz, 3H), 2.34 (br d, J=16.5, 5.9 Hz, 1H), 2.55 (br dd, J=16.5, 5.9 Hz, 1H), 3.23 (s, 3H), 4.01 (br d, J=18.3, 1H), 4.14 (dq, J=13.5, 6.8 Hz, 1H), 4.38-4.65 (m, 2H), 4.99-5.11 (m, 1H), 7.39 (dd, J=8.3, 1.9 Hz, 1H), 7.49-7.57 (m, 2H), 7.63 (d, J=2.0 Hz, 1H), 7.67 (d, J=8.1 Hz, 1H), 8.03-8.10 (m, 2H); LCMS (method E): Rt 1.67 min

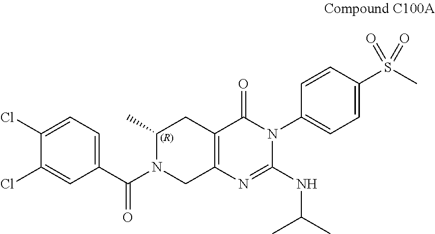

¹H NMR (400 MHz, DMSO-d₆, 126° C.) δ ppm 1.05-1.14 (m, 6H), 1.21 (d, J=6.8 Hz, 3H), 2.34 (br d, J=16.3 Hz, 1H), 2.55 (br dd, J=16.6, 6.1 Hz, 1H), 3.23 (s, 3H), 4.01 (br d, J=18.7 Hz, 1H), 4.16 (dq, J=13.6, 6.7 Hz, 1H), 4.41-4.64 (m, 2H), 5.09 (br d, J=7.5 Hz, 1H), 7.39 (dd, J=8.1, 2.0 Hz, 1H), 7.51-7.58 (m, 2H), 7.65 (d, J=2.0 Hz, 1H), 7.69 (d, J=8.1 Hz, 1H), 8.05-8.11 (m, 2H); LCMS (method D): Rt 1.95 min

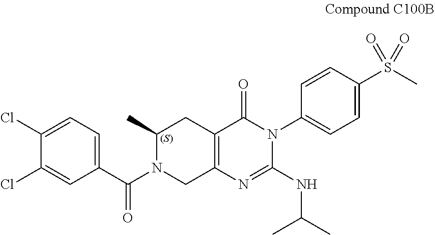

¹H NMR (400 MHz, DMSO-d₆, 126° C.) δ ppm 1.05-1.12 (m, 6H), 1.20 (d, J=6.6 Hz, 3H), 2.34 (br d, J=16.5 Hz, 1H), 2.55 (br dd, J=16.7, 6.2 Hz, 1H), 3.24 (s, 3H), 4.01 (br d, J=18.7 Hz, 1H), 4.15 (dq, J=13.6, 6.8 Hz, 1H), 4.40-4.64 (m, 2H), 5.08 (br d, J=7.5 Hz, 1H), 7.40 (dd, J=8.4, 2.0 Hz, 1H), 7.50-7.58 (m, 2H), 7.64 (d, J=1.8 Hz, 1H), 7.68 (d, J=8.1 Hz, 1H), 8.04-8.12 (m, 2H); LCMS (method D): Rt 1.93 min

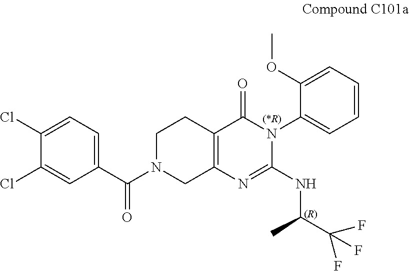

¹H NMR (400 MHz, DMSO-d₆, 80° C.) δ ppm 1.22 (d, J=7.1 Hz, 3H), 2.41 (t, J=5.8 Hz, 2H), 3.47-3.74 (m, 2H), 3.76 (s, 3H), 4.33 (br s, 2H), 4.88-5.12 (m, 1H), 5.71 (br d, J=8.8 Hz, 1H), 7.07-7.14 (m, 2H), 7.24 (br d, J=8.1 Hz, 1H), 7.45 (dd, J=8.3, 1.9 Hz, 1H), 7.51 (ddd, J=8.3, 6.2, 3.1 Hz, 1H), 7.67-7.73 (m, 2H); LCMS (method A): Rt 2.01 min

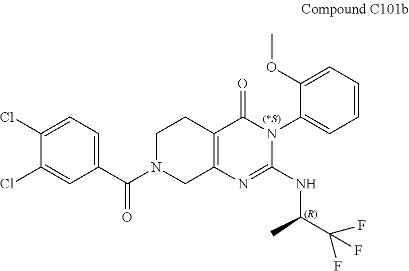

¹H NMR (400 MHz, DMSO-d₆, 80° C.) δ ppm 1.22 (d, J=7.1 Hz, 3H), 2.41 (t, J=5.5 Hz, 2H), 3.72 (s, 3H), 3.54-3.70 (m, 2H), 4.13-4.47 (m, 2H), 4.88-5.09 (m, 1H), 5.68 (br d, J=9.0 Hz, 1H), 7.07-7.13 (m, 1H), 7.20 (dd, J=7.8, 1.7 Hz, 1H), 7.22 (dd, J=8.4, 1.1 Hz, 1H), 7.45 (dd, J=8.4, 2.0 Hz, 1H), 7.50 (ddd, J=8.4, 7.5, 1.8 Hz, 1H), 7.67-7.73 (m, 2H); LCMS (method A): Rt 2.00 min

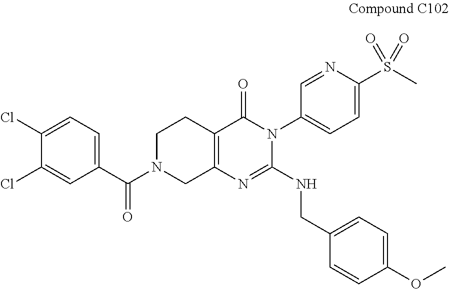 [SMILES: COc1ccc(CNc2nc3c(c(=O)n2-c2ccc(S(C)(=O)=O)nc2)CCN(C(=O)c2ccc(Cl)c(Cl)c2)C3)cc1]

¹H NMR (400 MHz, DMSO-d₆, 100° C.) δ ppm 2.42 (t, J=5.9 Hz, 2H), 3.30 (s, 3H), 3.59-3.68 (m, 2H), 3.72 (s, 3H), 4.30 (s, 2H), 4.36 (d, J=5.9 Hz, 2H), 6.75 (br t, J=5.7 Hz, 1H), 6.80-6.84 (m, 2H), 7.17 (br d, J=8.6 Hz, 2H), 7.41 (dd, J=8.4, 2.0 Hz, 1H), 7.65-7.72 (m, 2H), 8.10 (dd, J=8.4, 2.4 Hz, 1H), 8.19 (dd, J=8.1, 0.7 Hz, 1H), 8.68 (dd, J=2.4, 0.7 Hz, 1H); LCMS (method C): Rt 1.09 min

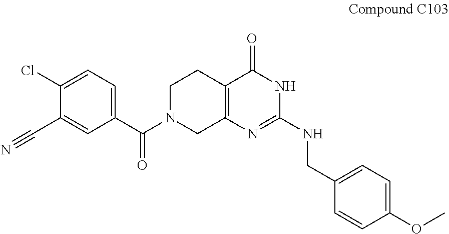 [SMILES: COc1ccc(CNc2nc3c(c(=O)[nH]2)CCN(C(=O)c2ccc(Cl)c(C#N)c2)C3)cc1]

¹H NMR (400 MHz, DMSO-d₆, 126° C.) δ ppm 2.38 (t, J=5.8 Hz, 2H), 3.58 (br t, J=5.8 Hz, 2H), 3.74 (s, 3H), 4.23 (s, 2H), 4.36 (br d, J=3.5 Hz, 2H), 6.42 (br s, 1H), 6.84-6.89 (m, 2H), 7.20-7.25 (m, 2H), 7.71-7.78 (m, 2H), 7.95 (d, J=1.8 Hz, 1H), 10.35 (br s, 1H); LCMS (method B): Rt 1.63 min

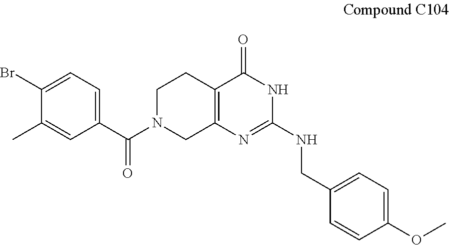

¹H NMR (400 MHz, DMSO-d₆, 126° C.) δ ppm 2.36 (t, J=5.8 Hz, 2H), 2.39 (s, 3H), 3.58 (t, J=5.8 Hz, 2H), 3.74 (s, 3H), 4.22 (s, 2H), 4.36 (br s, 2H), 6.40 (br s, 1H), 6.84-6.89 (m, 2H), 7.14 (dd, J=8.1, 2.0 Hz, 1H), 7.20-7.24 (m, 2H), 7.36 (d, J=1.8 Hz, 1H), 7.61 (d, J=8.1 Hz, 1H), 10.30 (br s, 1H); LCMS (method C): Rt 1.01 min

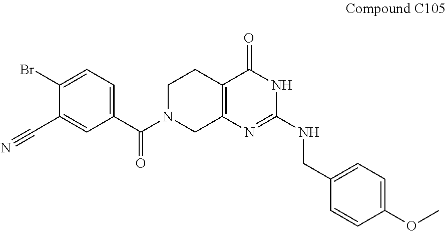 [SMILES: COc1ccc(CNc2nc3c(c(=O)[nH]2)CCN(C(=O)c2ccc(Br)c(C#N)c2)C3)cc1]

¹H NMR (400 MHz, DMSO-d₆, 126° C.) δ ppm 2.38 (t, J=5.8 Hz, 2H), 3.58 (br t, J=5.6 Hz, 2H), 3.74 (s, 3H), 4.22 (s, 2H), 4.36 (d, J=4.8 Hz, 2H), 6.35-6.46 (m, 1H), 6.84-6.89 (m, 2H), 7.20-7.24 (m, 2H), 7.64 (dd, J=8.3, 2.1 Hz, 1H), 7.88-7.92 (m, 1H), 10.32 (s, 1H); LCMS (method C): Rt 0.90 min

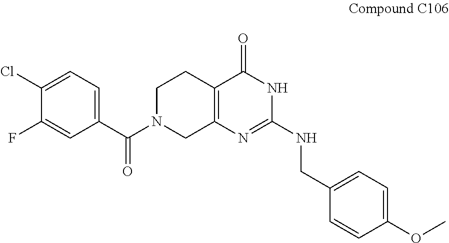 [SMILES: COc1ccc(CNc2nc3c(c(=O)[nH]2)CCN(C(=O)c2ccc(Cl)c(F)c2)C3)cc1]

¹H NMR (400 MHz, DMSO-d₆, 126° C.) δ ppm 2.37 (br t, J=5.7 Hz, 2H), 3.59 (br t, J=5.8 Hz, 2H), 3.74 (s, 3H), 4.22 (s, 2H), 4.36 (br d, J=4.0 Hz, 2H), 6.41 (br s, 1H), 6.86 (d, J=8.8 Hz, 2H), 7.22 (d, J=8.6 Hz, 2H), 7.26 (dd, J=8.1, 1.1 Hz, 1H), 7.40 (dd, J=9.7, 1.8 Hz, 1H), 7.60 (t, J=7.8 Hz, 1H), 10.28 (br s, 1H); LCMS (method B): Rt 1.78 min

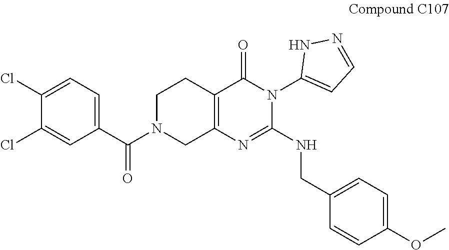 [SMILES: COc1ccc(CNc2nc3c(c(=O)n2-c2ccn[nH]2)CCN(C(=O)c2ccc(Cl)c(Cl)c2)C3)cc1]

¹H NMR (400 MHz, DMSO-d₆, 100° C.) δ ppm 2.40 (t, J=5.8 Hz, 2H), 3.59-3.68 (m, 2H), 3.72 (s, 3H), 4.27 (s, 2H), 4.36 (d, J=5.9 Hz, 2H), 6.23-6.33 (m, 2H), 6.79-6.84 (m, 2H), 7.16 (br d, J=8.3 Hz, 2H), 7.41 (dd, J=8.1, 2.0 Hz, 1H), 7.64-7.69 (m, 2H), 7.82 (s, 1H), 12.92 (s, 1H); LCMS (method F): Rt 1.83 min

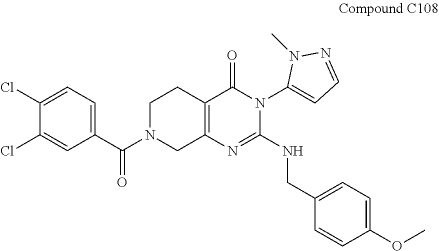 [SMILES: COc1ccc(CNc2nc3c(c(=O)n2-c2ccnn2C)CCN(C(=O)c2ccc(Cl)c(Cl)c2)C3)cc1]

¹H NMR (400 MHz, DMSO-d₆, 100° C.) δ ppm 2.37-2.45 (m, 2H), 3.53 (s, 3H), 3.57-3.66 (m, 2H), 3.72 (s, 3H), 4.23-4.42 (m, 4H), 6.30 (d, J=2.0 Hz, 1H), 6.61 (br t, J=5.6 Hz, 1H), 6.79-6.87 (m, 2H), 7.17 (br d, J=8.6 Hz, 2H), 7.41 (dd, J=8.1, 2.0 Hz, 1H), 7.55 (d, J=2.0 Hz, 1H), 7.64-7.71 (m, 2H); LCMS (method C): Rt 1.21 min

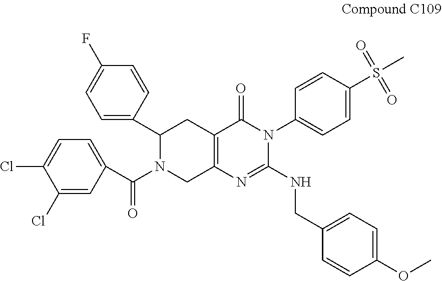 [SMILES: COc1ccc(CNc2nc3c(c(=O)n2-c2ccc(S(C)(=O)=O)cc2)CC(c2ccc(F)cc2)N(C(=O)c2ccc(Cl)c(Cl)c2)C3)cc1]

¹H NMR (400 MHz, DMSO-d₆, 101° C.) δ ppm 2.79-2.89 (m, 1H), 3.07 (br d, J=16.7 Hz, 1H), 3.25 (s, 3H), 3.71 (s, 3H), 3.75 (d, J=18.3 Hz, 1H), 4.24 (dd, J=14.6, 5.5 Hz, 1H), 4.34 (dd, J=14.4, 5.6 Hz, 1H), 4.39-4.67 (m, 1H), 5.43-5.75 (m, 1H), 6.31-6.43 (m, 1H), 6.78 (d, J=8.6 Hz, 2H), 7.06-7.18 (m, 4H), 7.22-7.32 (m, 2H), 7.45 (dd, J=8.3, 2.0 Hz, 1H), 7.55 (d, J=8.5 Hz, 2H), 7.69 (d, J=8.1 Hz, 1H), 7.71 (d, J=1.8 Hz, 1H), 8.08 (d, J=8.5 Hz, 2H); LCMS (method C): Rt 1.33 min

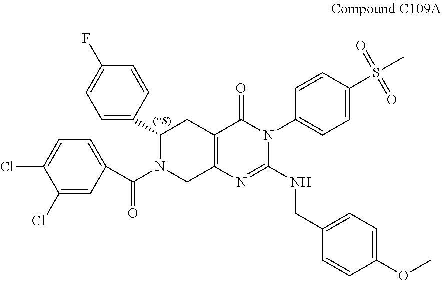 [SMILES: COc1ccc(CNc2nc3c(c(=O)n2-c2ccc(S(C)(=O)=O)cc2)C[C@@H](c2ccc(F)cc2)N(C(=O)c2ccc(Cl)c(Cl)c2)C3)cc1]

¹H NMR (400 MHz, DMSO-d₆, 81° C.) δ ppm 2.84 (br dd, J=16.7, 5.9 Hz, 1H), 2.98-3.14 (m, 1H), 3.25 (s, 3H), 3.70 (s, 3H), 3.74 (d, J=18.7 Hz, 1H), 4.05-4.67 (m, 1H), 4.23 (dd, J=15.0, 5.7 Hz, 1H), 4.34 (dd, J=14.7, 5.9 Hz, 1H), 5.20-6.07 (m, 1H), 6.46 (t, J=5.9 Hz, 1H), 6.78 (d, J=8.6 Hz, 2H), 7.05-7.19 (m, 4H), 7.22-7.32 (m, 2H), 7.46 (dd, J=8.3, 1.9 Hz, 1H), 7.56 (d, J=8.4 Hz, 2H), 7.69 (d, J=8.4 Hz, 1H), 7.73 (d, J=1.8 Hz, 1H), 8.08 (d, J=8.6 Hz, 2H); LCMS (method D): Rt 2.13 min

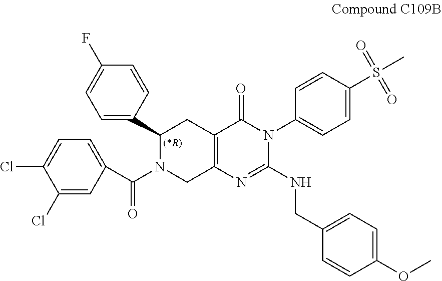 [SMILES: COc1ccc(CNc2nc3c(c(=O)n2-c2ccc(S(C)(=O)=O)cc2)C[C@H](c2ccc(F)cc2)N(C(=O)c2ccc(Cl)c(Cl)c2)C3)cc1]

¹H NMR (400 MHz, DMSO-d₆, 81° C.) δ ppm 2.84 (br dd, J=16.8, 6.1 Hz, 1H), 2.94-3.13 (m, 1H), 3.26 (s, 3H), 3.70 (s, 3H), 3.70-3.80 (m, 1H), 4.08-4.82 (m, 1H), 4.22 (dd, J=15.0, 5.7 Hz, 1H), 4.34 (dd, J=14.8, 5.9 Hz, 1H), 5.24-5.97 (m, 1H), 6.48 (t, J=5.9 Hz, 1H), 6.78 (d, J=8.4 Hz, 2H), 7.05-7.19 (m, 4H), 7.21-7.34 (m, 2H), 7.46 (dd, J=8.2, 2.0 Hz, 1H), 7.56 (d, J=8.4 Hz, 2H), 7.69 (d, J=8.1 Hz, 1H), 7.73 (d, J=2.0 Hz, 1H), 8.08 (d, J=8.8 Hz, 2H); LCMS (method D): Rt 2.13 min

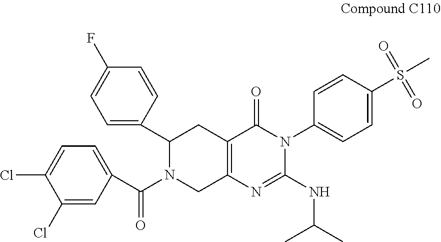 [SMILES: CC(C)Nc1nc2c(c(=O)n1-c1ccc(S(C)(=O)=O)cc1)CC(c1ccc(F)cc1)N(C(=O)c1ccc(Cl)c(Cl)c1)C2]

¹H NMR (400 MHz, DMSO-d₆, 126° C.) δ ppm 1.01 (d, J=6.6 Hz, 3H), 1.04 (d, J=6.6 Hz, 3H), 2.79-2.89 (m, 1H), 3.07 (d, J=16.7, 1H), 3.22 (s, 3H), 3.79 (d, J=18.5 Hz, 1H), 4.06 (dq, J=14.3, 6.5 Hz, 1H), 4.48 (br d, J=18.1 Hz, 1H), 5.12 (br d, J=7.5 Hz, 1H), 5.51-5.73 (m, 1H), 7.06-7.16 (m, 2H), 7.21-7.31 (m, 2H), 7.43 (dd, J=8.1, 2.0 Hz, 1H), 7.47-7.54 (m, 2H), 7.61-7.73 (m, 2H), 8.02-8.09 (m, 2H); LCMS (method C): Rt 1.32 min

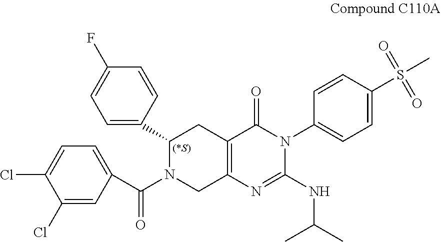 [SMILES: CC(C)Nc1nc2c(c(=O)n1-c1ccc(S(C)(=O)=O)cc1)C[C@@H](c1ccc(F)cc1)N(C(=O)c1ccc(Cl)c(Cl)c1)C2]

¹H NMR (400 MHz, DMSO-d₆, 126° C.) δ ppm 1.01 (d, J=6.4 Hz, 3H), 1.04 (d, J=6.4 Hz, 3H), 2.84-2.93 (m, 1H), 3.07 (br d, J=16.7 Hz), 3.23 (s, 3H), 3.80 (d, J=18.5 Hz, 1H), 4.08 (dq, J=13.6, 6.8 Hz, 1H), 4.48 (br d, J=18.3 Hz, 1H), 5.13 (br d, J=7.3 Hz, 1H), 5.57-5.69 (m, 1H), 7.09-7.18 (m, 2H), 7.24-7.33 (m, 2H), 7.45 (dd, J=8.1, 2.0 Hz, 1H), 7.49-7.54 (m, 2H), 7.66-7.73 (m, 2H), 8.04-8.09 (m, 2H); LCMS (method D): Rt 2.11 min

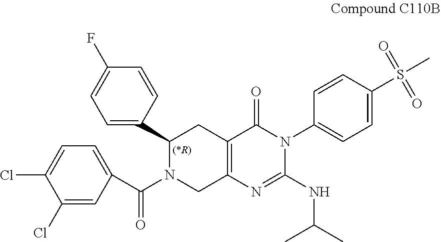 [SMILES: CC(C)Nc1nc2c(c(=O)n1-c1ccc(S(C)(=O)=O)cc1)C[C@H](c1ccc(F)cc1)N(C(=O)c1ccc(Cl)c(Cl)c1)C2]

¹H NMR (400 MHz, DMSO-d₆, 101° C.) δ ppm 1.01 (d, J=6.5 Hz, 3H), 1.03 (d, J=6.5 Hz, 3H), 3.08 (d, J=17.0 Hz, 1H), 3.25 (s, 3H), 3.77 (d, J=18.1 Hz, 1H), 4.07 (sext, J=6.6 Hz, 1H), 4.39-4.67 (m, 1H), 5.28 (d, J=7.9 Hz, 1H), 5.43-5.75 (m, 1H), 7.06-7.18 (m, 2H), 7.22-7.32 (m, 2H), 7.45 (dd, J=8.3, 2.0 Hz, 1H), 7.50-7.54 (m, 2H), 7.69 (d, J=8.1 Hz, 1H), 7.72 (d, J=2.0 Hz, 1H), 8.08-8.10 (m, 2H); LCMS (method D): Rt 2.11 min

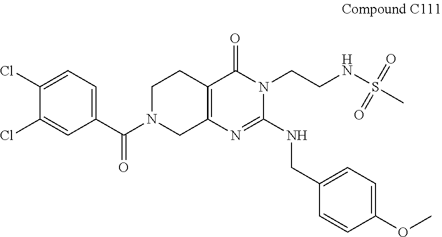 [SMILES: COc1ccc(CNc2nc3c(c(=O)n2CCNS(C)(=O)=O)CCN(C(=O)c2ccc(Cl)c(Cl)c2)C3)cc1]

¹H NMR (400 MHz, CDCl₃, 56° C.) δ ppm 2.52 (br t, J=5.3 Hz, 2H), 2.89 (s, 3H), 3.32 (t, J=6.8 Hz, 2H), 3.47-3.75 (m, 2H), 3.77 (s, 3H), 4.08 (t, J=6.8 Hz, 2H), 4.38 (br s, 2H), 4.51 (br s, 2H), 6.03 (br s, 1H), 6.83 (d, J=8.6 Hz, 2H), 7.19-7.32 (m, 3H), 7.50 (d, J=8.1 Hz, 1H), 7.54 (d, J=2.0 Hz, 1H); LCMS (method C): Rt 1.12 min

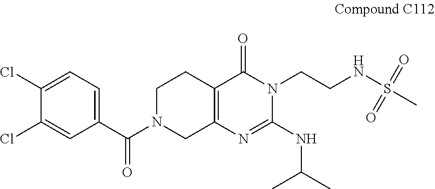 [SMILES: CC(C)Nc1nc2c(c(=O)n1CCNS(C)(=O)=O)CCN(C(=O)c1ccc(Cl)c(Cl)c1)C2]

¹H NMR (400 MHz, CDCl₃, 56° C.) δ ppm 1.24 (d, J=6.6 Hz, 6H), 2.55 (br t, J=5.8 Hz, 2H), 3.00 (s, 3H), 3.39 (t, J=6.7 Hz, 2H), 3.51-3.83 (m, 2H), 4.08 (t, J=6.7 Hz, 2H), 4.14-4.25 (m, 1H), 4.26-4.53 (m, 2H), 4.69 (br s, 1H), 5.48 (br d, J=6.8 Hz, 1H), 7.27 (dd, J=8.1 Hz, 1H), 7.50 (d, J=8.4 Hz, 1H), 7.54 (d, J=2.0 Hz, 1H); LCMS (method C): Rt1.10 min

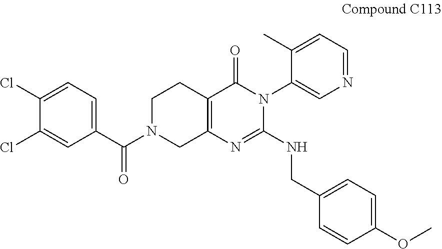 [SMILES: COc1ccc(CNc2nc3c(c(=O)n2-c2cnccc2C)CCN(C(=O)c2ccc(Cl)c(Cl)c2)C3)cc1]

¹H NMR (400 MHz, DMSO-d₆, 100° C.) δ ppm 2.01 (s, 3H), 2.42 (t, J=5.8 Hz, 2H), 3.62 (br t, J=4.6 Hz, 2H), 3.72 (s, 3H), 4.26-4.39 (m, 4H), 6.46 (br s, 1H), 6.79-6.84 (m, 2H), 7.15 (d, J=8.6 Hz, 2H), 7.40-7.44 (m, 2H), 7.67 (s, 1H), 7.68 (d, J=5.9 Hz, 1H), 8.29 (s, 1H), 8.52 (d, J=5.0 Hz, 1H); LCMS (method C): Rt 1.19 min

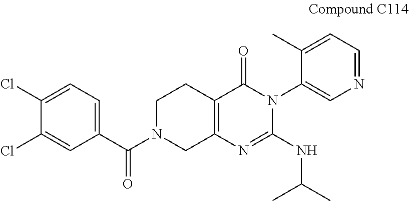 [SMILES: Cc1ccncc1-n1c(NC(C)C)nc2c(c1=O)CCN(C(=O)c1ccc(Cl)c(Cl)c1)C2]

¹H NMR (400 MHz, DMSO-d₆, 100° C.) δ ppm 1.05 (d, J=6.6 Hz, 3H), 1.06 (d, J=6.6 Hz, 3H), 2.04 (s, 3H), 2.42 (br t, J=5.9 Hz, 2H), 3.64 (t, J=4.7 Hz, 2H), 4.16 (dq, J=13.6, 6.6 Hz, 1H), 4.33 (br s, 2H), 5.37 (br d, J=7.5 Hz, 1H), 7.41-7.45 (m, 2H), 7.68 (s, 1H), 7.69 (d, J=6.2 Hz, 1H), 8.26 (s, 1H), 8.52 (d, J=5.1 Hz, 1H); LCMS (method B14001B7014): Rt 1.89 min

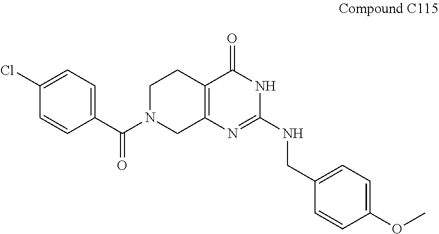 [SMILES: COc1ccc(CNc2nc3c(c(=O)[nH]2)CCN(C(=O)c2ccc(Cl)cc2)C3)cc1]

¹H NMR (400 MHz, DMSO-d₆, 126° C.) δ ppm 2.37 (t, J=5.9 Hz, 2H), 3.59 (t, J=5.8 Hz, 2H), 3.74 (s, 3H), 4.22 (s, 2H), 4.36 (br s, 2H), 6.40 (br s, 1H), 6.84-6.89 (m, 2H), 7.20-7.24 (m, 2H), 7.40-7.49 (m, 4H), 10.29 (br s, 1H); LCMS (method A): Rt 1.58 min

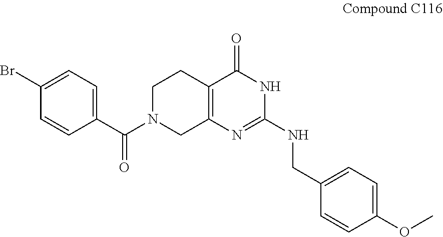 [SMILES: COc1ccc(CNc2nc3c(c(=O)[nH]2)CCN(C(=O)c2ccc(Br)cc2)C3)cc1]

¹H NMR (400 MHz, DMSO-d₆, 126° C.) δ ppm 2.37 (t, J=5.9 Hz, 2H), 3.58 (t, J=5.8 Hz, 2H), 3.74 (s, 3H), 4.22 (s, 2H), 4.36 (s, 2H), 6.40 (br s, 1H), 6.84-6.89 (m, 2H), 7.20-7.24 (m, 2H), 7.33-7.38 (m, 2H), 7.59-7.63 (m, 2H), 10.30 (br s, 1H); LCMS (method A): Rt 1.61 min

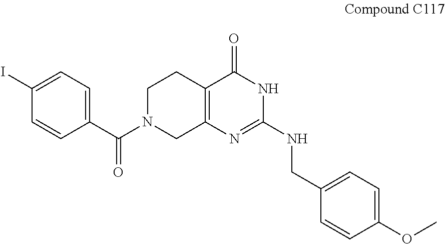 [SMILES: COc1ccc(CNc2nc3c(c(=O)[nH]2)CCN(C(=O)c2ccc(I)cc2)C3)cc1]

¹H NMR (400 MHz, DMSO-d₆, 126° C.) δ ppm 2.36 (t, J=5.8 Hz, 2H), 3.58 (t, J=5.8 Hz, 2H), 3.74 (s, 3H), 4.22 (s, 2H), 4.36 (s, 2H), 6.40 (br s, 1H), 6.84-6.89 (m, 2H), 7.17-7.25 (m, 4H), 7.78-7.83 (m, 2H), 10.30 (br s, 1H); LCMS (method A): Rt 1.65 min

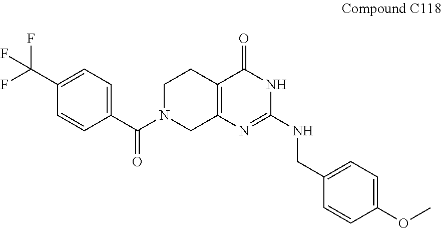 [SMILES: COc1ccc(CNc2nc3c(c(=O)[nH]2)CCN(C(=O)c2ccc(C(F)(F)F)cc2)C3)cc1]

¹H NMR (400 MHz, DMSO-d₆, 150° C.) δ ppm 2.39 (tt, J=5.8, 1.3 Hz, 2H), 3.59 (t, J=5.8 Hz, 2H), 3.74 (s, 3H), 4.23 (s, 2H), 4.36 (s, 2H), 6.36 (br s, 1H), 6.84-6.88 (m, 2H), 7.19-7.24 (m, 2H), 7.61 (d, J=7.9 Hz, 2H), 7.75 (d, J=7.9 Hz, 2H), 10.24 (s, 1H); LCMS (method B): Rt 1.80 min

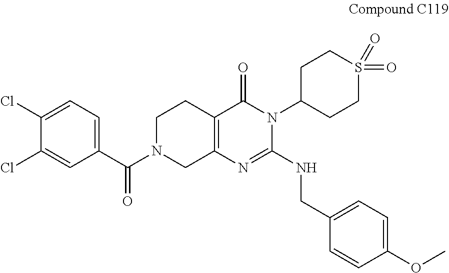 [SMILES: COc1ccc(CNc2nc3c(c(=O)n2C2CCS(=O)(=O)CC2)CCN(C(=O)c2ccc(Cl)c(Cl)c2)C3)cc1]

¹H NMR (400 MHz, DMSO-d₆, 100° C.) δ ppm 1.88-1.98 (m, 2H), 2.36 (t, J=5.9 Hz, 2H), 3.03-3.15 (m, 2H), 3.20-3.32 (m, 4H), 3.50-3.62 (m, 2H), 3.73 (s, 3H), 4.18 (s, 2H), 4.39-4.51 (m, 3H), 6.83-6.88 (m, 2H), 7.21-7.31 (m, 3H), 7.38 (dd, J=8.3, 1.9 Hz, 1H), 7.63 (d, J=2.0 Hz, 1H), 7.66 (d, J=8.4 Hz, 1H); LCMS (method C): Rt 1.20 min

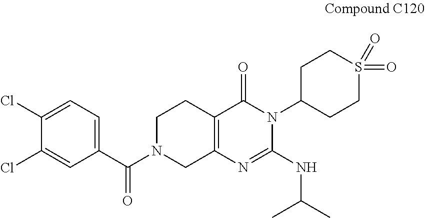 [SMILES: CC(C)Nc1nc2c(c(=O)n1C1CCS(=O)(=O)CC1)CCN(C(=O)c1ccc(Cl)c(Cl)c1)C2]

¹H NMR (400 MHz, DMSO-d₆, 100° C.) δ ppm 1.20 (d, J=6.6 Hz, 6H), 1.86-1.94 (m, 2H), 2.36 (t, J=5.9 Hz, 2H), 3.04-3.12 (m, 2H), 3.18-3.36 (m, 4H), 3.57 (br t, J=4.6 Hz, 2H), 4.11-4.23 (m, 3H), 4.38-4.46 (m, 1H), 6.22 (br d, J=6.6 Hz, 1H), 7.39 (dd, J=8.1, 2.0 Hz, 1H), 7.64 (d, J=1.8 Hz, 1H), 7.67 (d, J=8.1 Hz, 1H); LCMS (method C): Rt 1.11 min

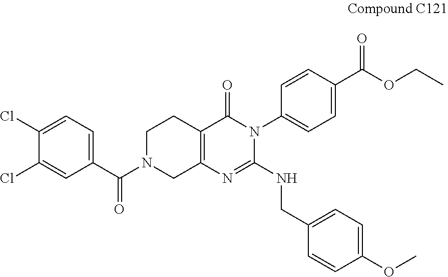 [SMILES: CCOC(=O)c1ccc(-n2c(NCc3ccc(OC)cc3)nc3c(c2=O)CCN(C(=O)c2ccc(Cl)c(Cl)c2)C3)cc1]

¹H NMR (400 MHz, DMSO-d₆, 100° C.) δ ppm 1.34 (t, J=7.1 Hz, 3H), 2.41 (t, J=5.8 Hz, 2H), 3.62 (br t, J=5.5 Hz, 2H), 3.71 (s, 3H), 4.28 (br s, 2H), 4.33 (d, J=5.7 Hz, 2H), 4.37 (q, J=7.0 Hz, 2H), 6.21 (br t, J=5.7 Hz, 1H), 6.78-6.83 (m, 2H), 7.14 (d, J=8.6 Hz, 2H), 7.38-7.43 (m, 3H), 7.64-7.70 (m, 2H), 8.07-8.11 (m, 2H); LCMS (method C): Rt 1.28 min

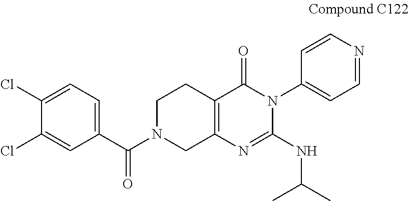 [SMILES: CC(C)Nc1nc2c(c(=O)n1-c1ccncc1)CCN(C(=O)c1ccc(Cl)c(Cl)c1)C2]

¹H NMR (400 MHz, DMSO-d₆, 100° C.) δ ppm 1.06 (d, J=6.6 Hz, 6H), 2.41 (t, J=5.8 Hz, 2H), 3.63 (t, J=4.5 Hz, 2H), 4.14 (dq, J=13.8, 6.7 Hz, 1H), 4.30 (s, 2H), 5.29 (br d, J=7.7 Hz, 1H), 7.28-7.32 (m, 2H), 7.42 (dd, J=8.1, 2.0 Hz, 1H), 7.67 (d, J=2.0 Hz, 1H), 7.69 (d, J=8.1 Hz, 1H), 8.73-8.77 (m, 2H); LCMS (method C): Rt 0.98 min

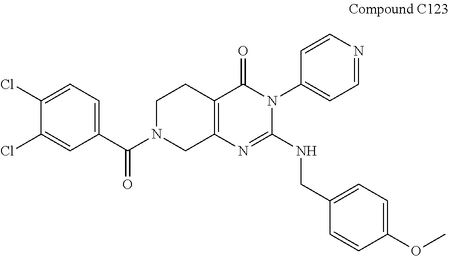 [SMILES: COc1ccc(CNc2nc3c(c(=O)n2-c2ccncc2)CCN(C(=O)c2ccc(Cl)c(Cl)c2)C3)cc1]

¹H NMR (400 MHz, DMSO-d₆, 100° C.) δ ppm 2.41 (t, J=5.9 Hz, 2H), 3.62 (br t, J=5.7 Hz, 2H), 3.72 (s, 3H), 4.28 (s, 2H), 4.33 (d, J=5.7 Hz, 2H), 6.33 (t, J=5.7 Hz, 1H), 6.79-6.83 (m, 2H), 7.13-7.18 (m, 2H), 7.30-7.33 (m, 2H), 7.41 (dd, J=8.1, 2.0 Hz, 1H), 7.65 (d, J=2.0 Hz, 1H), 7.67 (d, J=8.1 Hz, 1H), 8.74-8.77 (m, 2H); LCMS (method C): Rt 1.04 min

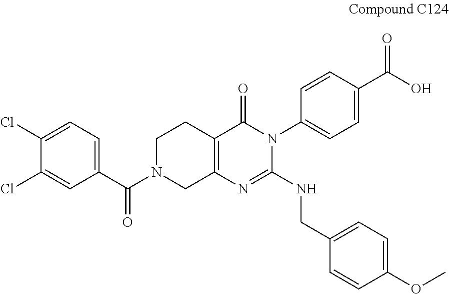 [SMILES: COc1ccc(CNc2nc3c(c(=O)n2-c2ccc(C(=O)O)cc2)CCN(C(=O)c2ccc(Cl)c(Cl)c2)C3)cc1]

¹H NMR (400 MHz, DMSO-d₆, 100° C.) δ ppm 2.41 (t, J=5.8 Hz, 2H), 3.62 (br t, J=5.0 Hz, 2H), 3.71 (s, 3H), 4.28 (s, 2H), 4.33 (d, J=5.7 Hz, 2H), 6.19 (br t, J=5.8 Hz, 1H), 6.78-6.83 (m, 2H), 7.14 (d, J=8.6 Hz, 2H), 7.34-7.39 (m, 2H), 7.41 (dd, J=8.4, 2.0 Hz, 1H), 7.65-7.69 (m, 2H), 8.05-8.10 (m, 2H); LCMS (method C): Rt 0.85 min

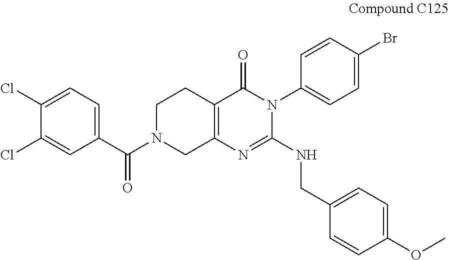 [SMILES: COc1ccc(CNc2nc3c(c(=O)n2-c2ccc(Br)cc2)CCN(C(=O)c2ccc(Cl)c(Cl)c2)C3)cc1]

¹H NMR (400 MHz, DMSO-d₆, 100° C.) δ ppm 2.40 (t, J=5.8 Hz, 2H), 3.61 (br t, J=4.8 Hz, 2H), 3.72 (s, 3H), 4.27 (s, 2H), 4.33 (d, J=5.9 Hz, 2H), 6.26 (br t, J=5.6 Hz, 1H), 6.78-6.83 (m, 2H), 7.15 (br d, J=8.6 Hz, 2H), 7.18-7.23 (m, 2H), 7.41 (dd, J=8.1, 2.0 Hz, 1H), 7.66 (d, J=2.0 Hz, 1H), 7.67 (d, J=8.4 Hz, 1H), 7.69-7.73 (m, 2H); LCMS (method C): Rt 1.23 min

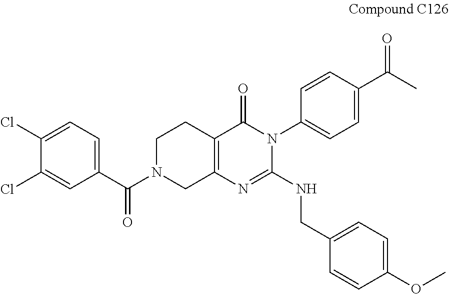 [SMILES: COc1ccc(CNc2nc3c(c(=O)n2-c2ccc(C(C)=O)cc2)CCN(C(=O)c2ccc(Cl)c(Cl)c2)C3)cc1]

¹H NMR (400 MHz, DMSO-d₆, 100° C.) δ ppm 2.41 (t, J=5.8 Hz, 2H), 2.61 (s, 3H), 3.62 (br t, J=5.1 Hz, 2H), 3.71 (s, 3H), 4.29 (s, 2H), 4.33 (d, J=5.7 Hz, 2H), 6.17 (br t, J=5.4 Hz, 1H), 6.78-6.83 (m, 2H), 7.15 (d, J=8.4 Hz, 2H), 7.36-7.45 (m, 3H), 7.65-7.70 (m, 2H), 8.06-8.11 (m, 2H); LCMS (method C): Rt 1.13 min

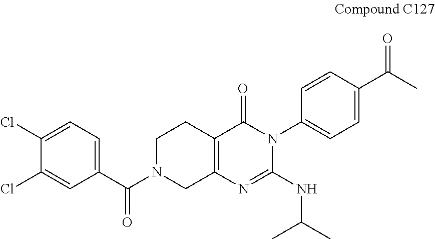 [SMILES: CC(=O)c1ccc(-n2c(NC(C)C)nc3c(c2=O)CCN(C(=O)c2ccc(Cl)c(Cl)c2)C3)cc1]

¹H NMR (400 MHz, DMSO-d₆, 100° C.) δ ppm 8.06-8.11 (m, 2H), 7.66-7.71 (m, 2H), 7.43 (dd, J=8.1, 2.0 Hz, 1H), 7.36-7.41 (m, 2H), 5.01 (br d, J=7.3 Hz, 1H), 4.31 (s, 2H), 4.13 (dq, J=13.9, 6.7 Hz, 1H), 3.64 (br t, J=5.5 Hz, 2H), 2.62 (s, 3H), 2.41 (t, J=5.8 Hz, 2H), 1.05 (d, J=6.6 Hz, 6H); LCMS (method C): Rt 1.11 min

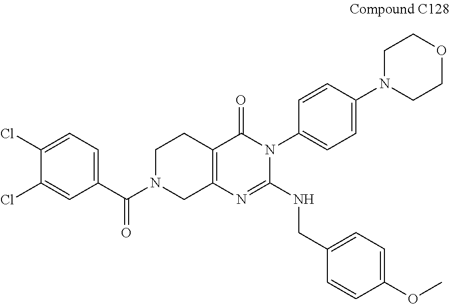 [SMILES: COc1ccc(CNc2nc3c(c(=O)n2-c2ccc(N4CCOCC4)cc2)CCN(C(=O)c2ccc(Cl)c(Cl)c2)C3)cc1]

¹H NMR (400 MHz, DMSO-d₆, 100° C.) δ ppm 2.40 (t, J=5.8 Hz, 2H), 3.18-3.23 (m, 4H), 3.56-3.67 (m, 2H), 3.71 (s, 3H), 3.73-3.77 (m, 4H), 4.26 (s, 2H), 4.34 (br d, J=4.6 Hz, 2H), 5.87-5.93 (m, 1H), 6.78-6.83 (m, 2H), 7.01-7.07 (m, 4H), 7.14 (d, J=8.6 Hz, 2H), 7.41 (dd, J=8.2, 2.0 Hz, 1H), 7.66 (d, J=2.0 Hz, 1H), 7.67 (d, J=8.1 Hz, 1H); LCMS (method C): Rt 1.14 min

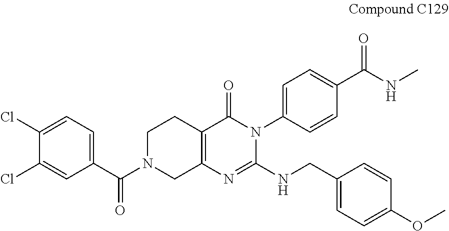 [SMILES: CNC(=O)c1ccc(-n2c(NCc3ccc(OC)cc3)nc3c(c2=O)CCN(C(=O)c2ccc(Cl)c(Cl)c2)C3)cc1]

¹H NMR (400 MHz, DMSO-d₆, 100° C.) δ ppm 2.41 (t, J=5.9 Hz, 2H), 2.82 (d, J=4.6 Hz, 3H), 3.58-3.66 (m, 2H), 3.71 (s, 3H), 4.28 (s, 2H), 4.33 (d, J=5.9 Hz, 2H), 6.11 (t, J=5.7 Hz, 1H), 6.77-6.83 (m, 2H), 7.14 (d, J=8.6 Hz, 2H), 7.28-7.34 (m, 2H), 7.41 (dd, J=8.1, 2.0 Hz, 1H), 7.64-7.70 (m, 2H), 7.94-8.00 (m, 2H), 8.16-8.24 (m, 1H); LCMS (method C): Rt 1.01 min

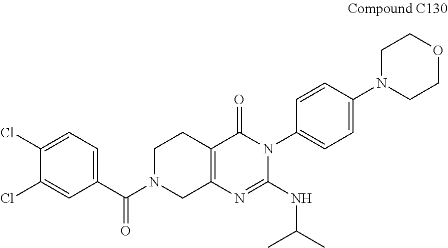 [SMILES: CC(C)Nc1nc2c(c(=O)n1-c1ccc(N3CCOCC3)cc1)CCN(C(=O)c1ccc(Cl)c(Cl)c1)C2]

¹H NMR (400 MHz, DMSO-d₆, 100° C.) δ ppm 1.04 (d, J=6.5 Hz, 6H), 2.40 (t, J=5.9 Hz, 2H), 3.17-3.26 (m, 4H), 3.58-3.67 (m, 2H), 3.72-3.79 (m, 4H), 4.08 (dq, J=13.6, 6.8 Hz, 1H), 4.29 (br s, 2H), 4.71 (br d, J=7.7 Hz, 1H), 7.04 (s, 4H), 7.42 (dd, J=8.4, 2.0 Hz, 1H), 7.65-7.71 (m, 2H); LCMS (method B): Rt 2.14 min

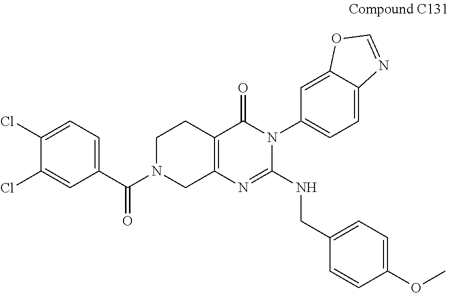 [SMILES: COc1ccc(CNc2nc3c(c(=O)n2-c2ccc4ncoc4c2)CCN(C(=O)c2ccc(Cl)c(Cl)c2)C3)cc1]

¹H NMR (400 MHz, DMSO-d₆, 100° C.) δ ppm 2.39-2.44 (m, 2H), 3.58-3.67 (m, 2H), 3.71 (s, 3H), 4.23-4.38 (m, 4H), 6.24-6.30 (m, 1H), 6.77-6.85 (m, 2H), 7.14 (d, J=8.6 Hz, 2H), 7.24 (dd, J=8.5, 1.9 Hz, 1H), 7.42 (dd, J=8.1, 2.0 Hz, 1H), 7.66-7.71 (m, 2H), 7.74 (d, J=2.0 Hz, 1H), 7.91 (d, J=8.4 Hz, 1H), 8.73 (s, 1H); LCMS (method C): Rt 1.08 min

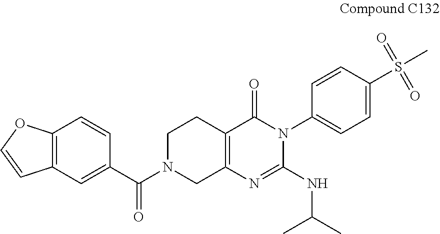 [SMILES: CC(C)Nc1nc2c(c(=O)n1-c1ccc(S(C)(=O)=O)cc1)CCN(C(=O)c1ccc3occc3c1)C2]

¹H NMR (400 MHz, DMSO-d₆, 100° C.) δ ppm 1.05 (d, J=6.4 Hz, 6H), 2.42 (t, J=5.8 Hz, 2H), 3.25 (s, 3H), 3.68 (t, J=5.8 Hz, 2H), 4.13 (dq, J=13.8, 6.7 Hz, 1H), 4.36 (s, 2H), 5.21 (br d, J=7.7 Hz, 1H), 6.99 (dd, J=2.2, 0.9 Hz, 1H), 7.39 (dd, J=8.5, 1.7 Hz, 1H), 7.50-7.55 (m, 2H), 7.63 (d, J=8.6 Hz, 1H), 7.76 (d, J=1.3 Hz, 1H), 7.99 (d, J=2.2 Hz, 1H), 8.04-8.10 (m, 2H); LCMS (method B): Rt 1.77 min

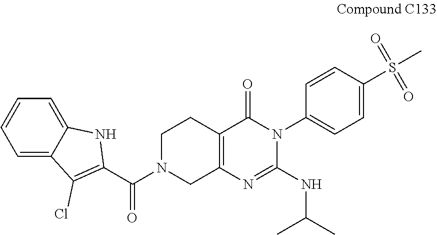 [SMILES: CC(C)Nc1nc2c(c(=O)n1-c1ccc(S(C)(=O)=O)cc1)CCN(C(=O)c1[nH]c3ccccc3c1Cl)C2]

¹H NMR (400 MHz, DMSO-d₆, 150° C.) δ ppm 1.07 (d, J=6.6 Hz, 6H), 2.47-2.52 (m, 2H), 3.22 (s, 3H), 3.79 (t, J=5.9 Hz, 2H), 4.12 (dq, J=13.8, 6.8 Hz, 1H), 4.40 (s, 2H), 4.91-4.96 (m, 1H), 7.13-7.18 (m, 1H), 7.22-7.28 (m, 1H), 7.44 (d, J=8.1 Hz, 1H), 7.52-7.61 (m, 3H), 8.04-8.09 (m, 2H), 11.39 (br s, 1H); LCMS (method B): Rt 1.96 min

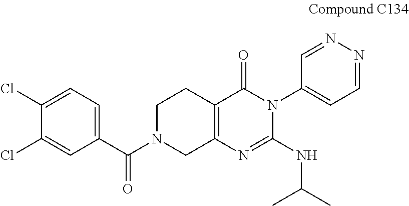 [SMILES: CC(C)Nc1nc2c(c(=O)n1-c1ccnnc1)CCN(C(=O)c1ccc(Cl)c(Cl)c1)C2]

¹H NMR (400 MHz, DMSO-d₆, 100° C.) δ ppm 1.07 (d, J=6.6 Hz, 6H), 2.41 (t, J=5.9 Hz, 2H), 3.64 (br t, J=5.5 Hz, 2H), 4.16 (dq, J=13.5, 6.8 Hz, 1H), 4.31 (s, 2H), 5.86 (br d, J=8.1 Hz, 1H), 7.42 (dd, J=8.1, 2.0 Hz, 1H), 7.66-7.72 (m, 3H), 9.14 (dd, J=2.4, 1.1 Hz, 1H), 9.40 (dd, J=5.5, 1.1 Hz, 1H); LCMS (method C): Rt 0.94 min

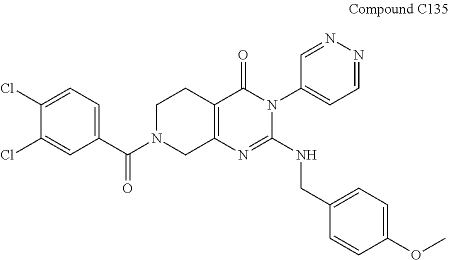 [SMILES: COc1ccc(CNc2nc3c(c(=O)n2-c2ccnnc2)CCN(C(=O)c2ccc(Cl)c(Cl)c2)C3)cc1]

¹H NMR (400 MHz, DMSO-d₆, 100° C.) δ ppm 2.41 (t, J=5.8 Hz, 2H), 3.62 (br t, J=5.1 Hz, 2H), 3.70 (s, 3H), 4.30 (s, 2H), 4.34 (s, 2H), 6.76 (br s, 1H), 6.79-6.85 (m, 2H), 7.17 (d, J=8.6 Hz, 2H), 7.41 (dd, J=8.3, 1.9 Hz, 1H), 7.66 (d, J=2.0 Hz, 1H), 7.68 (d, J=8.1 Hz, 1H), 7.74 (dd, J=5.5, 2.6 Hz, 1H), 9.19 (dd, J=2.4, 1.1 Hz, 1H), 9.41 (dd, J=5.5, 1.1 Hz, 1H); LCMS (method D): Rt 1.91 min

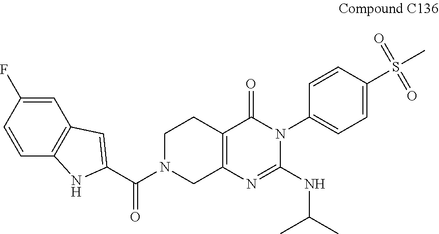 [SMILES: CC(C)Nc1nc2c(c(=O)n1-c1ccc(S(C)(=O)=O)cc1)CCN(C(=O)c1cc3cc(F)ccc3[nH]1)C2]

¹H NMR (400 MHz, DMSO-d₆, 150° C.) δ ppm 1.09 (d, J=6.4 Hz, 6H), 2.51 (t, J=5.8 Hz, 2H), 3.22 (s, 3H), 3.92 (t, J=5.9 Hz, 2H), 4.15 (dq, J=13.9, 6.6 Hz, 1H), 4.57 (t, J=1.3 Hz, 2H), 4.95 (br d, J=7.3 Hz, 1H), 6.83 (s, 1H), 6.99 (td, J=9.2, 2.4 Hz, 1H), 7.32 (dd, J=9.7, 2.4 Hz, 1H), 7.44 (dd, J=8.3, 4.6 Hz, 1H), 7.49-7.55 (m, 2H), 8.03-8.10 (m, 2H), 11.18 (br s, 1H); LCMS (method B): Rt 1.86 min

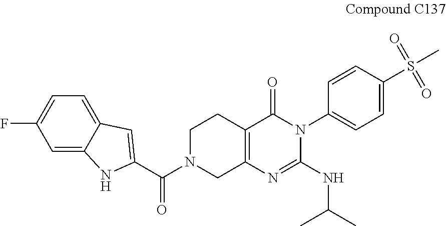 [SMILES: CC(C)Nc1nc2c(c(=O)n1-c1ccc(S(C)(=O)=O)cc1)CCN(C(=O)c1cc3ccc(F)cc3[nH]1)C2]

¹H NMR (400 MHz, DMSO-d₆, 150° C.) δ ppm 1.09 (d, J=6.6 Hz, 6H), 2.52 (t, J=5.9 Hz, 2H), 3.21 (s, 3H), 3.93 (t, J=5.8 Hz, 2H), 4.14 (dq, J=14.1, 6.8 Hz, 1H), 4.54 (m, 2H), 4.93 (br d, J=6.6 Hz, 1H), 6.85-6.95 (m, 2H), 7.16 (dd, J=10.0, 2.3 Hz, 1H), 7.50-7.56 (m, 2H), 7.61 (dd, J=8.8, 5.5 Hz, 1H), 8.04-8.10 (m, 2H), 11.14 (br s, 1H); LCMS (method B): Rt 1.92 min

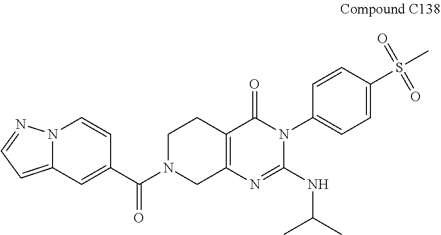 [SMILES: CC(C)Nc1nc2c(c(=O)n1-c1ccc(S(C)(=O)=O)cc1)CCN(C(=O)c1ccn3nccc3c1)C2]

¹H NMR (400 MHz, DMSO-d₆, 101° C.) δ ppm 1.06 (d, J=6.6 Hz, 6H), 2.45 (t, J=5.9 Hz, 2H), 3.25 (s, 3H), 3.71 (t, J=5.8 Hz, 2H), 4.14 (dq, J=14.0, 6.8 Hz, 1H), 4.37 (s, 2H), 5.23 (br d, J=7.7 Hz, 1H), 6.71 (dd, J=2.3, 0.8 Hz, 1H), 6.89 (dd, J=7.9, 1.8 Hz, 1H), 7.50-7.55 (m, 2H), 7.79 (dd, J=1.8, 1.1 Hz, 1H), 8.02 (d, J=2.2 Hz, 1H), 8.05-8.10 (m, 2H), 8.67 (dt, J=7.0, 0.9 Hz, 1H); LCMS (method B): Rt 1.58 min

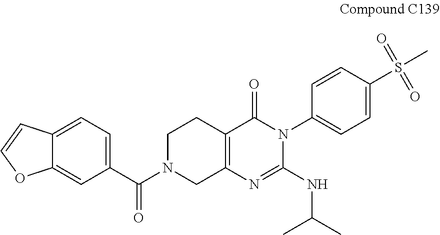 [SMILES: CC(C)Nc1nc2c(c(=O)n1-c1ccc(S(C)(=O)=O)cc1)CCN(C(=O)c1ccc3ccoc3c1)C2]

¹H NMR (400 MHz, DMSO-d₆, 101° C.) δ ppm 1.05 (d, J=6.6 Hz, 6H), 2.42 (t, J=5.8 Hz, 2H), 3.25 (s, 3H), 3.68 (t, J=5.5 Hz, 2H), 4.15 (dq, J=13.6, 6.6 Hz, 1H), 4.36 (s, 2H), 5.22 (br d, J=7.7 Hz, 1H), 6.99 (dd, J=2.2, 0.9 Hz, 1H), 7.33 (dd, J=7.9, 1.3 Hz, 1H), 7.50-7.56 (m, 2H), 7.66 (s, 1H), 7.73 (d, J=7.9 Hz, 1H), 8.01 (d, J=2.2 Hz, 1H), 8.05-8.09 (m, 2H); LCMS (method B): Rt 1.78 min

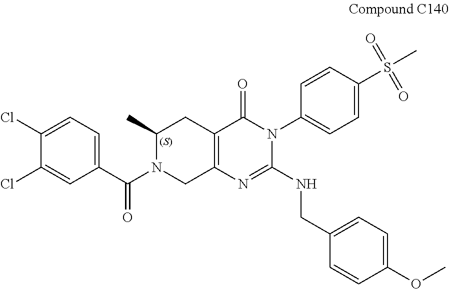

¹H NMR (400 MHz, DMSO-d₆, 80° C.) δ ppm 8.08 (d, J=8.6 Hz, 2H), 7.69 (d, J=8.1 Hz, 1H), 7.66 (d, J=1.8 Hz, 1H), 7.57 (br d, J=7.9 Hz, 2H), 7.35-7.42 (m, 3H), 6.92-6.98 (m, 2H), 6.44 (t, J=5.7 Hz, 1H), 4.65-4.20 (m, 4H), 3.99 (br d, J=18.3 Hz, 1H), 3.72 (s, 3H), 3.25 (s, 3H), 2.53 (dd, J=16.5, 5.9 Hz, 1H), 2.32 (d, J=16.3 Hz, 1H), 1.17 (d, J=6.6 Hz, 3H); LCMS (method C): Rt 1.11 min

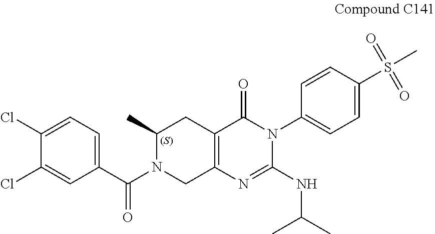

¹H NMR (400 MHz, DMSO-d₆, 80° C.) δ ppm 8.07 (d, J=8.8 Hz, 2H), 7.70 (d, J=8.1 Hz, 1H), 7.68 (d, J=2.0 Hz, 1H), 7.54 (d, J=8.4 Hz, 2H), 7.41 (dd, J=1.9, 8.2 Hz, 1H), 5.37 (br d, J=7.9 Hz, 1H), 4.23-4.76 (m, 2H), 4.16 (dq, J=13.8, 6.7 Hz, 1H), 4.02 (br d, J=19.2 Hz, 1H), 3.26 (s, 3H), 2.53 (dd, J=16.3, 5.9 Hz, 1H), 2.32 (br d, J=16.3 Hz, 1H), 1.18 (d, J=6.8 Hz, 3H), 1.07 (d, J=6.6 Hz, 3H), 1.06 (d, J=6.6 Hz, 3H); LCMS (method C): Rt 1.08 min

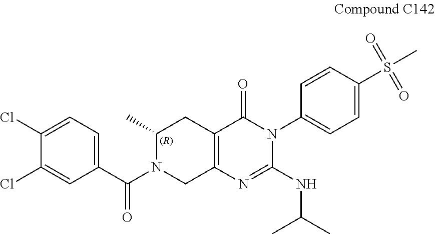

¹H NMR (400 MHz, DMSO-d₆, 80° C.) δ ppm 8.07 (d, J=8.8 Hz, 2H), 7.70 (d, J=8.4 Hz, 1H), 7.68 (d, J=2.0 Hz, 1H), 7.54 (br d, J=8.6 Hz, 2H), 7.42 (dd, J=2.0, 8.1 Hz, 1H), 5.37 (br d, J=7.7 Hz, 1H), 4.83-4.26 (m, 2H), 4.16 (qd, J=6.6, 13.8 Hz, 1H), 4.02 (br d, J=19.2 Hz, 1H), 3.26 (s, 3H), 2.60-2.50 (m, 1H), 2.32 (d, J=16.3 Hz, 1H), 1.18 (d, J=6.8 Hz, 3H), 1.07 (d, J=6.4 Hz, 3H), 1.06 (d, J=6.4 Hz, 3H); LCMS (method C): Rt 1.07 min

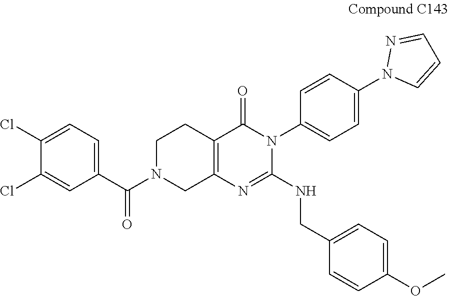 [SMILES: COc1ccc(CNc2nc3c(c(=O)n2-c2ccc(-n4cccn4)cc2)CCN(C(=O)c2ccc(Cl)c(Cl)c2)C3)cc1]

¹H NMR (400 MHz, DMSO-d₆, 100° C.) δ ppm 2.42 (t, J=5.8 Hz, 2H), 3.59-3.67 (m, 2H), 3.71 (s, 3H), 4.29 (s, 2H), 4.35 (d, J=5.9 Hz, 2H), 6.23 (br t, J=5.7 Hz, 1H), 6.54 (dd, J=2.5, 1.9 Hz, 1H), 6.78-6.84 (m, 2H), 7.16 (d, J=8.6 Hz, 2H), 7.33-7.38 (m, 2H), 7.42 (dd, J=8.1, 2.0 Hz, 1H), 7.65-7.69 (m, 3H), 7.92-8.03 (m, 2H), 8.45 (d, J=2.4 Hz, 1H); LCMS (method C): Rt 1.15 min

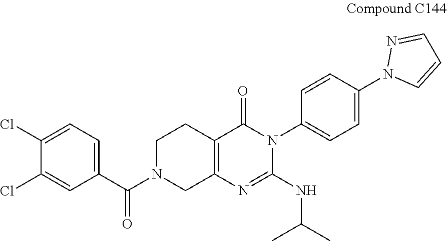 [SMILES: CC(C)Nc1nc2c(c(=O)n1-c1ccc(-n3cccn3)cc1)CCN(C(=O)c1ccc(Cl)c(Cl)c1)C2]

¹H NMR (400 MHz, DMSO-d₆, 100° C.) δ ppm 1.06 (br d, J=6.4 Hz, 6H), 2.30-2.46 (m, 2H), 3.48-3.77 (m, 2H), 4.05-4.20 (m, 1H), 4.32 (s, 2H), 5.07 (br d, J=6.8 Hz, 1H), 6.55 (s, 1H), 7.34 (br d, J=8.1 Hz, 2H), 7.44 (br d, J=8.4 Hz, 1H), 7.61-7.73 (m, 2H), 7.76 (s, 1H), 7.98 (br d, J=7.9 Hz, 2H), 8.47 (br s, 1H); LCMS (method C): Rt 1.14 min

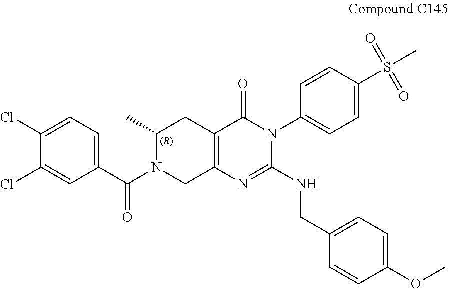

¹H NMR (400 MHz, DMSO-d₆, 80° C.) δ ppm 8.06-8.09 (m, 2H), 7.69 (d, J=8.4 Hz, 1H), 7.66 (d, J=2.0 Hz, 1H), 7.57 (br d, J=7.9 Hz, 2H), 7.40 (dd, J=1.9, 8.3 Hz, 1H), 7.16 (d, J=8.4 Hz, 2H), 6.82 (br d, J=8.6 Hz, 2H), 6.49-6.40 (m, 1H), 4.70-4.23 (m, 4H), 4.10-3.90 (m, 1H), 3.72 (s, 3H), 3.25 (s, 3H), 2.60-2.50 (m, 1H), 2.32 (br d, J=16.1 Hz, 1H), 1.17 (d, J=6.8 Hz, 3H); LCMS (method C): Rt 1.10 min

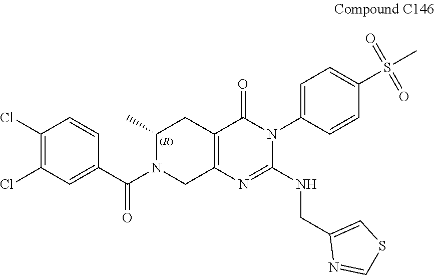

¹H NMR (400 MHz, DMSO-d₆, 80° C.) δ ppm 8.94 (d, J=1.8 Hz, 1H), 8.09 (d, J=8.8 Hz, 2H), 7.69 (d, J=8.1 Hz, 1H), 7.66 (d, J=2.0 Hz, 1H), 7.62 (br d, J=8.4 Hz, 2H), 7.40 (dd, J=1.9, 8.3 Hz, 1H), 7.38-7.34 (m, 1H), 6.49 (br s, 1H), 4.70-4.19 (m, 4H), 3.98 (br d, J=18.7 Hz, 1H), 3.26 (s, 3H), 2.54 (br dd, J=16.8, 5.6 Hz, 1H), 2.33 (d, J=15.9 Hz, 1H), 1.17 (d, J=6.8 Hz, 3H); LCMS (method C): Rt 0.97 min

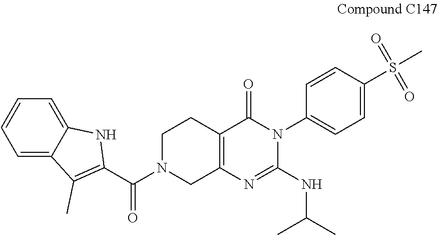 [SMILES: Cc1c(C(=O)N2CCc3c(nc(NC(C)C)n(-c4ccc(S(C)(=O)=O)cc4)c3=O)C2)[nH]c2ccccc12]

¹H NMR (400 MHz, DMSO-d₆, 101° C.) δ ppm 1.05 (d, J=6.6 Hz, 6H), 2.30 (s, 3H), 2.44-2.46 (m, 2H), 3.25 (s, 3H), 3.75 (t, J=5.9 Hz, 2H), 4.13 (dq, J=13.7, 6.7 Hz, 1H), 4.38 (s, 2H), 5.23 (br d, J=7.3 Hz, 1H), 7.01-7.08 (m, 1H), 7.16 (td, J=7.5, 1.0 Hz, 1H), 7.36 (d, J=8.1 Hz, 1H), 7.50-7.58 (m, 3H), 8.04-8.10 (m, 2H), 10.91 (br s, 1H); LCMS (method B): Rt 1.89 min

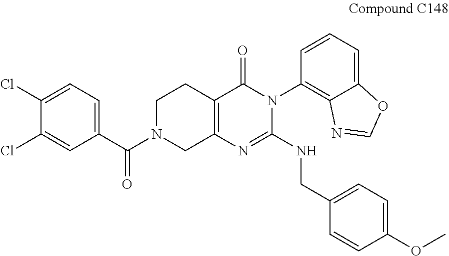 [SMILES: COc1ccc(CNc2nc3c(c(=O)n2-c2cccc4ocnc24)CCN(C(=O)c2ccc(Cl)c(Cl)c2)C3)cc1]

¹H NMR (400 MHz, DMSO-d₆, 100° C.) δ ppm 2.42 (t, J=5.8 Hz, 2H), 3.59-3.68 (m, 2H), 3.71 (s, 3H), 4.24-4.37 (m, 4H), 6.29 (br t, J=5.7 Hz, 1H), 6.75-6.81 (m, 2H), 7.11 (d, J=8.6 Hz, 2H), 7.32 (dd, J=7.8, 0.8 Hz, 1H), 7.43 (dd, J=8.4, 2.0 Hz, 1H), 7.58 (t, J=8.0 Hz, 1H), 7.65-7.70 (m, 2H), 7.87 (dd, J=8.3, 0.8 Hz, 1H), 8.66 (s, 1H); LCMS (method C): Rt 1.09 min

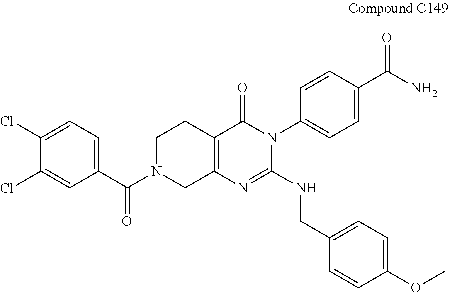 [SMILES: COc1ccc(CNc2nc3c(c(=O)n2-c2ccc(C(N)=O)cc2)CCN(C(=O)c2ccc(Cl)c(Cl)c2)C3)cc1]

¹H NMR (400 MHz, DMSO-d₆, 100° C.) δ ppm 2.41 (t, J=5.8 Hz, 2H), 3.62 (br t, J=5.1 Hz, 2H), 3.71 (s, 3H), 4.28 (s, 2H), 4.33 (s, 2H), 6.14 (br s, 1H), 6.77-6.85 (m, 2H), 7.14 (d, J=8.6 Hz, 2H), 7.29-7.34 (m, 2H), 7.41 (dd, J=8.1, 2.0 Hz, 3H), 7.65-7.69 (m, 2H), 7.99-8.05 (m, 2H); LCMS (method C): Rt 1.00 min

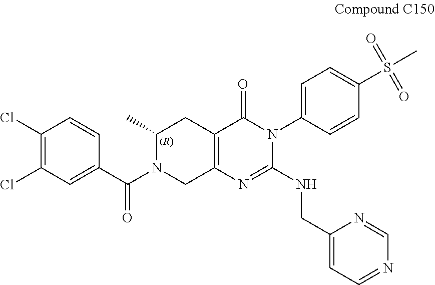

¹H NMR (400 MHz, DMSO-d₆, 81° C.) δ ppm 1.17 (d, J=6.8 Hz, 3H), 2.33 (d, J=16.1 Hz, 1H), 2.52-2.62 (m, 1H), 3.27 (s, 3H), 3.90 (br d, J=18.7 Hz, 1H), 4.14-4.80 (m, 4H), 6.59 (br t, J=5.2 Hz, 1H), 7.36-7.44 (m, 2H), 7.62-7.77 (m, 4H), 8.10-8.14 (m, 2H). 8.66 (d, J=5.3 Hz, 1H), 9.03 (d, J=1.1 Hz, 1H); LCMS (method C): Rt 0.89 min

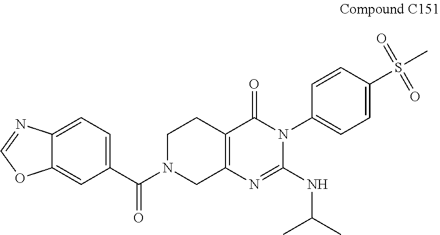 [SMILES: CC(C)Nc1nc2c(c(=O)n1-c1ccc(S(C)(=O)=O)cc1)CCN(C(=O)c1ccc3ncoc3c1)C2]

¹H NMR (400 MHz, DMSO-d₆, 150° C.) δ ppm 1.07 (d, J=6.4 Hz, 6H), 2.41-2.47 (m, 2H), 3.22 (s, 3H), 3.68 (t, J=5.8 Hz, 2H), 4.05-4.17 (m, 1H), 4.35 (t, J=1.2 Hz, 2H), 4.91 (br d, J=6.4 Hz, 1H), 7.46 (dd, J=8.1, 1.5 Hz, 1H), 7.49-7.54 (m, 2H), 7.79 (d, J=0.9 Hz, 1H), 7.82 (d, J=8.4 Hz, 1H), 8.02-8.09 (m, 2H), 8.62 (s, 1H); LCMS (method D): Rt 1.59 min

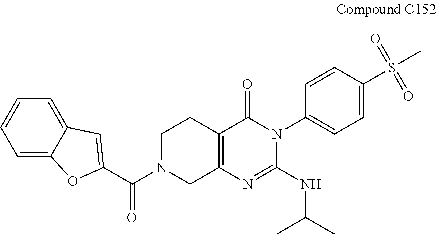 [SMILES: CC(C)Nc1nc2c(c(=O)n1-c1ccc(S(C)(=O)=O)cc1)CCN(C(=O)c1cc3ccccc3o1)C2]

¹H NMR (400 MHz, DMSO-d₆, 150° C.) δ ppm 1.09 (d, J=6.6 Hz, 6H), 2.52 (t, J=5.9 Hz, 2H), 3.22 (s, 3H), 3.91 (t, J=5.8 Hz, 2H), 4.16 (dq, J=14.0, 6.6 Hz, 1H), 4.51 (t, J=1.3 Hz, 2H), 4.98 (br d, J=5.3 Hz, 1H), 7.28-7.34 (m, 1H), 7.37 (d, J=0.9 Hz, 1H), 7.42 (ddd, J=8.4, 7.2, 1.3 Hz, 1H), 7.50-7.56 (m, 2H), 7.61 (dd, J=8.3, 0.8 Hz, 1H), 7.71-7.75 (m, 1H), 8.04-8.09 (m, 2H); LCMS (method B): Rt 1.87 min

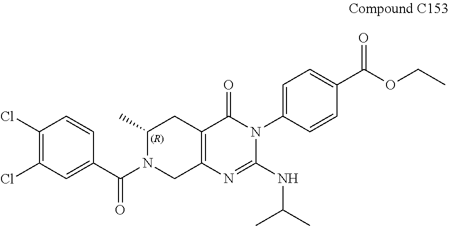

¹H NMR (400 MHz, DMSO-d₆, 81° C.) δ ppm 1.04 (d, J=6.6 Hz, 3H), 1.05 (d, J=6.6 Hz, 3H), 1.18 (d, J=6.8 Hz, 3H), 1.35 (t, J=7.1 Hz, 3H), 2.32 (br d, J=16.3 Hz, 1H), 2.50-2.58 (m, 1H), 4.02 (br d, J=18.9 Hz, 1H), 4.37 (q, J=7.0 Hz, 2H), 4.27-4.83 (m, 2H), 5.17 (br d, J=7.9 Hz, 1H), 7.34-7.47 (m, 3H), 7.67 (d, J=2.0 Hz, 1H), 7.70 (d, J=8.1 Hz, 1H), 8.09 (d, J=8.6 Hz, 2H); LCMS (method C): Rt 1.24 min

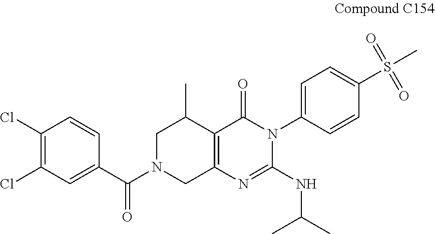 [SMILES: CC(C)Nc1nc2c(c(=O)n1-c1ccc(S(C)(=O)=O)cc1)C(C)CN(C(=O)c1ccc(Cl)c(Cl)c1)C2]

¹H NMR (400 MHz, DMSO-d₆, 81° C.) δ ppm 1.00-1.10 (m, 9H), 2.71-2.86 (m, 1H), 3.26 (s, 3H), 3.28-5.08 (m, 5H), 5.39 (br d, J=7.8 Hz, 1H), 7.43 (dd, J=8.2, 1.9 Hz, 1H), 7.50-7.55 (m, 2H), 7.68 (d, J=2.0 Hz, 1H), 7.71 (d, J=8.2 Hz, 1H), 8.04-7.09 (m, 2H); LCMS (method C): Rt 1.09 min

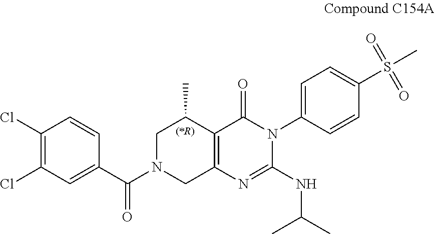 [SMILES: CC(C)Nc1nc2c(c(=O)n1-c1ccc(S(C)(=O)=O)cc1)[C@@H](C)CN(C(=O)c1ccc(Cl)c(Cl)c1)C2]

¹H NMR (400 MHz, DMSO-d₆, 81° C.) δ ppm 0.90-1.12 (m, 9H), 2.71-2.86 (m, 1H), 3.26 (s, 3H), 3.29-3.41 (m, 1H), 3.77-4.91 (m, 4H), 5.39 (br d, J=7.9 Hz, 1H), 7.43 (dd, J=8.2, 1.9 Hz, 1H), 7.50-7.54 (m, 2H), 7.68 (d, J=2.0 Hz, 1H), 7.71 (d, J=8.2 Hz, 1H), 8.03-8.10 (m, 2H); LCMS (method D): Rt 2.00 min

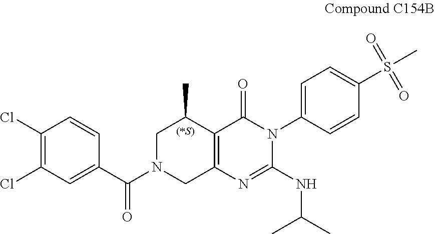 [SMILES: CC(C)Nc1nc2c(c(=O)n1-c1ccc(S(C)(=O)=O)cc1)[C@H](C)CN(C(=O)c1ccc(Cl)c(Cl)c1)C2]

¹H NMR (400 MHz, DMSO-d₆, 81° C.) δ ppm 0.91-1.12 (m, 9H), 2.70-2.86 (m, 1H), 3.26 (s, 3H), 3.28-3.44 (m, 1H), 3.66-5.12 (m, 4H), 5.39 (d, J=7.9 Hz, 1H), 7.43 (dd, J=8.2, 1.9 Hz, 1H), 7.50-7.55 (m, 2H), 7.68 (d, J=2.0 Hz, 1H), 7.71 (d, J=8.2 Hz, 1H), 8.04-8.10 (m, 2H); LCMS (method D): Rt 2.00 min

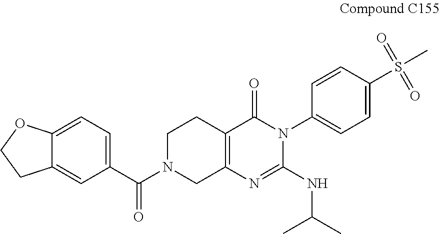 [SMILES: CC(C)Nc1nc2c(c(=O)n1-c1ccc(S(C)(=O)=O)cc1)CCN(C(=O)c1ccc3c(c1)CCO3)C2]

¹H NMR (400 MHz, DMSO-d₆, 150° C.) δ ppm 1.07 (d, J=6.4 Hz, 6H), 2.42 (t, J=5.9 Hz, 2H), 3.18-3.25 (m, 5H), 3.67 (t, J=5.9 Hz, 2H), 4.12 (dq, J=13.5, 6.6 Hz, 1H), 4.32 (t, J=1.3 Hz, 2H), 4.56 (t, J=8.7 Hz, 2H), 4.91 (br d, J=7.3 Hz, 1H), 6.76 (d, J=8.1 Hz, 1H), 7.14-7.22 (m, 1H), 7.31 (d, J=1.3 Hz, 1H), 7.48-7.55 (m, 2H), 8.03-8.10 (m, 2H); LCMS (method B): Rt 1.71 min

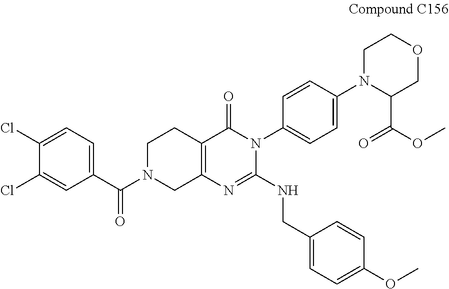 [SMILES: COC(=O)C1COCCN1c1ccc(-n2c(NCc3ccc(OC)cc3)nc3c(c2=O)CCN(C(=O)c2ccc(Cl)c(Cl)c2)C3)cc1]

¹H NMR (400 MHz, DMSO-d₆, 100° C.) δ ppm 2.40 (t, J=5.8 Hz, 2H), 3.34-3.43 (m, 1H), 3.45-3.53 (m, 1H), 3.55-3.64 (m, 3H), 3.63 (s, 3H), 3.71 (s, 3H), 3.78 (dd, J=11.7, 3.7 Hz, 1H), 3.95 (dd, J=11.2, 3.5 Hz, 1H), 4.23-4.28 (m, 3H), 4.34 (d, J=5.7 Hz, 2H), 4.59 (d, J=2.9 Hz, 1H), 5.79 (br t, J=5.6 Hz, 1H), 6.77-6.83 (m, 2H), 6.94-7.00 (m, 2H), 7.00-7.08 (m, 2H), 7.14 (d, J=8.6 Hz, 2H), 7.41 (dd, J=8.1, 2.0 Hz, 1H), 7.64-7.69 (m, 2H); LCMS (method C): Rt 1.15 min

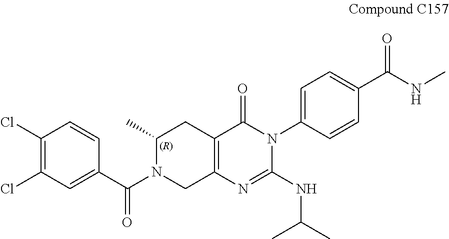

¹H NMR (400 MHz, DMSO-d₆, 81° C.) δ ppm 1.04 (d, J=6.6 Hz, 3H), 1.05 (d, J=6.6 Hz, 3H), 1.18 (d, J=6.8 Hz, 3H), 2.32 (d, J=16.5 Hz, 1H), 2.53 (br dd, J=16.6, 6.1 Hz, 1H), 2.83 (d, J=4.6 Hz, 3H), 4.02 (br d, J=18.5 Hz, 1H), 4.13 (dq, J=13.5, 6.7 Hz, 1H), 4.24-4.82 (m, 2H), 5.03 (br d, J=7.7 Hz, 1H), 7.33 (br d, J=8.1 Hz, 2H), 7.41 (dd, J=8.3, 1.9 Hz, 1H), 7.68 (d, J=1.8 Hz, 1H), 7.70 (d, J=8.1 Hz, 1H), 7.98 (d, J=8.8 Hz, 2H), 8.25-8.36 (m, 1H); LCMS (method B): Rt 1.92 min

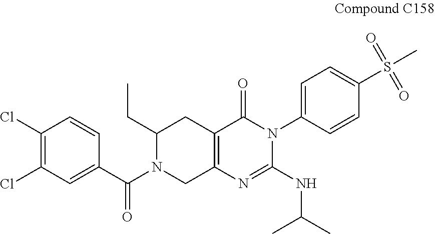 [SMILES: CCC1Cc2c(nc(NC(C)C)n(-c3ccc(S(C)(=O)=O)cc3)c2=O)CN1C(=O)c1ccc(Cl)c(Cl)c1]

¹H NMR (400 MHz, DMSO-d₆, 126° C.) δ ppm 0.84 (t, J=7.34 Hz, 3H), 1.07 (d, J=6.6 Hz, 3H), 1.07 (d, J=6.6 Hz, 3H), 1.42-1.54 (m, 1H), 1.58-1.71 (m, 1H), 2.43 (br d, J=16.5 Hz, 1H), 2.54 (ddt, J=16.6, 5.9, 1.8 Hz, 1H), 2.82 (s, 3H), 3.94 (br d, J=18.9 Hz, 1H), 4.14 (dq, J=13.5, 6.5 Hz, 1H), 4.24 (br s, 1H), 4.40-4.62 (m, 1H), 5.07 (br d, J=7.5 Hz, 1H), 7.39 (dd, J=8.3, 1.9 Hz, 1H), 7.50-7.55 (m, 2H), 7.61 (d, J=2.0 Hz, 1H), 7.67 (d, J=8.1 Hz, 1H), 8.04-8.09 (m, 2H); LCMS (method D): Rt 2.23 min

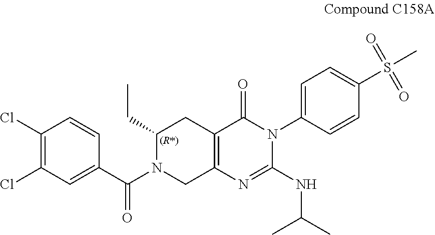 [SMILES: CC[C@@H]1Cc2c(nc(NC(C)C)n(-c3ccc(S(C)(=O)=O)cc3)c2=O)CN1C(=O)c1ccc(Cl)c(Cl)c1]

¹H NMR (400 MHz, DMSO-d₆, 126° C.) δ ppm 0.84 (t, J=7.4 Hz, 3H), 1.07 (d, J=6.6 Hz, 3H), 1.07 (d, J=6.6 Hz, 3H), 1.41-1.54 (m, 1H), 1.59-1.71 (m, 1H), 2.43 (br d, J=16.5 Hz, 1H), 2.54 (ddt, J=16.5, 6.2, 1.8 Hz, 1H), 2.82 (s, 3H), 3.94 (br d, J=18.7 Hz, 1H), 4.13 (dq, J=13.8, 6.6 Hz, 1H), 4.25 (br s, 1H), 4.40-4.62 (m, 1H), 5.06 (br d, J=7.3 Hz, 1H), 7.39 (dd, J=8.4, 2.0 Hz, 1H), 7.50-7.55 (m, 2H), 7.61 (d, J=1.8 Hz, 1H), 7.67 (d, J=8.1 Hz, 1H), 8.04-8.09 (m, 2H); LCMS (method B): Rt 2.12 min

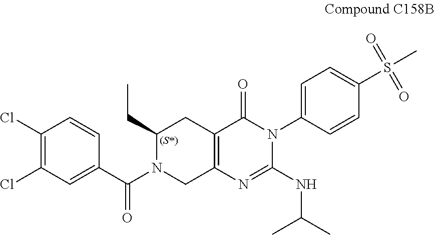 [SMILES: CC[C@H]1Cc2c(nc(NC(C)C)n(-c3ccc(S(C)(=O)=O)cc3)c2=O)CN1C(=O)c1ccc(Cl)c(Cl)c1]

¹H NMR (400 MHz, DMSO-d₆, 126° C.) δ ppm 0.84 (t, J=7.4 Hz, 3H), 1.07 (d, J=6.6 Hz, 3H), 1.07 (d, J=6.6 Hz, 3H), 1.42-1.55 (m, 1H), 1.59-1.70 (m, 1H), 2.43 (br d, J=16.5 Hz, 1H), 2.54 (ddt, J=16.5, 5.9, 1.8 Hz, 1H), 2.83 (s, 3H), 3.95 (br d, J=18.7 Hz, 1H), 4.14 (dq, J=13.8, 6.6 Hz, 1H), 4.24 (br s, 1H), 4.43-4.64 (m, 1H), 5.08 (br d, J=7.5 Hz, 1H), 7.39 (dd, J=8.4, 2.0 Hz, 1H), 7.50-7.55 (m, 2H), 7.62 (d, J=1.8 Hz, 1H), 7.67 (d, J=8.4 Hz, 1H), 8.05-8.09 (m, 2H); LCMS (method B): Rt 2.12 min

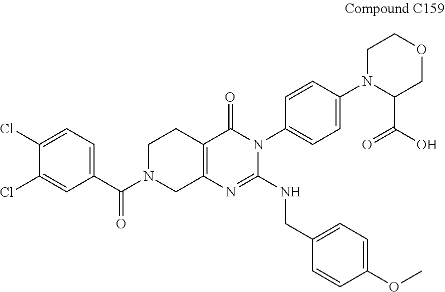 [SMILES: COc1ccc(CNc2nc3c(c(=O)n2-c2ccc(N4CCOCC4C(=O)O)cc2)CCN(C(=O)c2ccc(Cl)c(Cl)c2)C3)cc1]

¹H NMR (400 MHz, DMSO-d₆, 100° C.) δ ppm 2.39 (br t, J=5.7 Hz, 2H), 3.39-3.50 (m, 2H), 3.54-3.64 (m, 3H), 3.71 (s, 3H), 3.76 (dd, J=11.4, 3.7 Hz, 1H), 3.95 (br d, J=11.2 Hz, 1H), 4.23-4.31 (m, 3H), 4.34 (d, J=5.9 Hz, 2H), 4.43 (d, J=2.9 Hz, 1H), 5.83 (br t, J=5.7 Hz, 1H), 6.80 (d, J=8.6 Hz, 2H), 6.93-6.99 (m, 2H), 7.00-7.05 (m, 2H), 7.14 (d, J=8.6 Hz, 2H), 7.41 (dd, J=8.3, 1.9 Hz, 1H), 7.65-7.69 (m, 2H); LCMS (method C): Rt 0.86 min

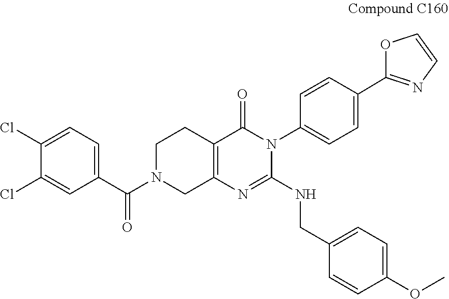 [SMILES: COc1ccc(CNc2nc3c(c(=O)n2-c2ccc(-c4ncco4)cc2)CCN(C(=O)c2ccc(Cl)c(Cl)c2)C3)cc1]

¹H NMR (400 MHz, DMSO-d₆, 100° C.) δ ppm 2.42 (br t, J=5.7 Hz, 2H), 3.59-3.66 (m, 2H), 3.71 (s, 3H), 4.29 (s, 2H), 4.34 (d, J=5.9 Hz, 2H), 6.24 (t, J=5.5 Hz, 1H), 6.77-6.84 (m, 2H), 7.15 (br d, J=8.6 Hz, 2H), 7.37 (d, J=0.4 Hz, 1H), 7.38-7.45 (m, 3H), 7.65-7.70 (m, 2H), 8.10-8.15 (m, 2H), 8.16 (s, 1H); LCMS (method C): Rt 1.13 min

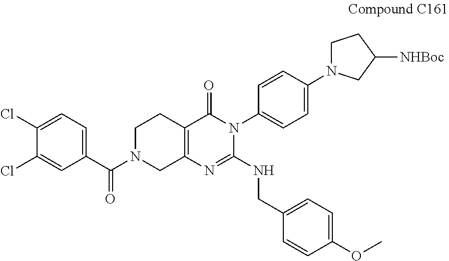 [SMILES: COc1ccc(CNc2nc3c(c(=O)n2-c2ccc(N4CCC(NC(=O)OC(C)(C)C)C4)cc2)CCN(C(=O)c2ccc(Cl)c(Cl)c2)C3)cc1]

¹H NMR (400 MHz, DMSO-d₆, 100° C.) δ ppm 1.40 (s, 9H), 1.88-1.99 (m, 1H), 2.13-2.23 (m, 1H), 2.39 (br t, J=5.6 Hz, 2H), 3.12 (dd, J=9.8, 5.2 Hz, 1H), 3.24-3.33 (m, 1H), 3.37-3.45 (m, 1H), 3.51 (dd, J=9.7, 6.6 Hz, 1H), 3.57-3.65 (m, 2H), 3.71 (s, 3H), 4.09-4.20 (m, 1H), 4.26 (br s, 2H), 4.33 (d, J=5.9 Hz, 2H), 5.80 (br t, J=5.5 Hz, 1H), 6.59-6.65 (m, 2H), 6.72 (br d, J=6.6 Hz, 1H), 6.78-6.83 (m, 2H), 6.94-7.00 (m, 2H), 7.14 (d, J=8.6 Hz, 2H), 7.41 (dd, J=8.3, 1.9 Hz, 1H), 7.65-7.70 (m, 2H); LCMS (method C): Rt 1.28 min

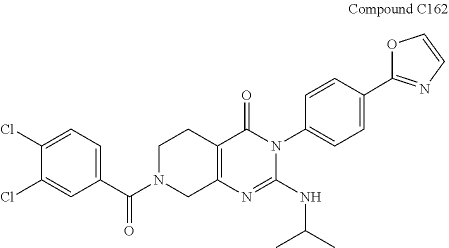 [SMILES: CC(C)Nc1nc2c(c(=O)n1-c1ccc(-c3ncco3)cc1)CCN(C(=O)c1ccc(Cl)c(Cl)c1)C2]

¹H NMR (400 MHz, DMSO-d₆, 100° C.) δ ppm 1.05 (d, J=6.6 Hz, 6H), 2.42 (br t, J=5.7 Hz, 2H), 3.59-3.70 (m, 2H), 4.14 (dq, J=13.6, 6.9 Hz, 1H), 4.32 (br s, 2H), 5.08 (br d, J=7.7 Hz, 1H), 7.35-7.41 (m, 3H), 7.43 (dd, J=8.1, 2.0 Hz, 1H), 7.65-7.73 (m, 2H), 8.09-8.19 (m, 3H); LCMS (method C): Rt 1.13 min

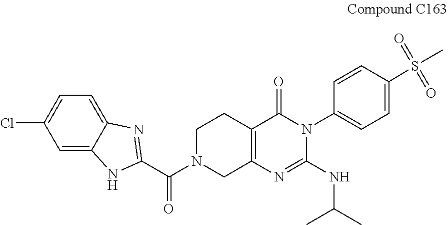 [SMILES: CC(C)Nc1nc2c(c(=O)n1-c1ccc(S(C)(=O)=O)cc1)CCN(C(=O)c1nc3ccc(Cl)cc3[nH]1)C2]

¹H NMR (400 MHz, DMSO-d₆, 150° C.) δ ppm 1.10 (d, J=6.6 Hz, 6H), 2.53 (t, J=5.8 Hz, 2H), 3.22 (s, 3H), 4.10-4.31 (m, 3H), 4.72-4.90 (m, 2H), 4.91-4.97 (m, 1H), 7.23-7.32 (m, 1H), 7.50-7.55 (m, 2H), 7.59-7.80 (m, 2H), 8.0.4-8.10 (m, 2H), 12.64-12.97 (m, 1H): LCMS (method B): Rt 1.87 min

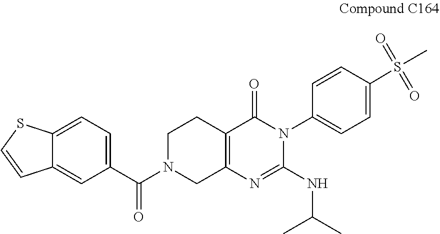 [SMILES: CC(C)Nc1nc2c(c(=O)n1-c1ccc(S(C)(=O)=O)cc1)CCN(C(=O)c1ccc3sccc3c1)C2]

¹H NMR (400 MHz, DMSO-d₆, 101° C.) δ ppm 1.05 (d, J=6.4 Hz, 6H), 2.43 (t, J=5.9 Hz, 2H), 3.25 (s, 3H), 3.69 (br t, J=5.7 Hz, 2H), 4.13 (dq, J=13.8, 6.7 Hz, 1H), 4.37 (s, 2H), 5.22 (br d, J=7.5 Hz, 1H), 7.42 (dd, J=8.1, 1.5 Hz, 1H), 7.51-7.58 (m, 3H), 7.80 (d, J=5.5 Hz, 1H), 7.97 (d, J=1.1 Hz, 1H), 8.05-8.13 (m, 3H); LCMS (method B): Rt 1.86 min

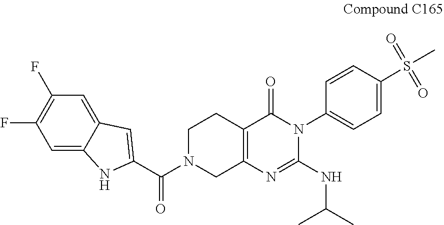 [SMILES: CC(C)Nc1nc2c(c(=O)n1-c1ccc(S(C)(=O)=O)cc1)CCN(C(=O)c1cc3cc(F)c(F)cc3[nH]1)C2]

¹H NMR (400 MHz, DMSO-d₆, 150° C.) δ ppm 1.09 (d, J=6.6 Hz, 6H), 2.51 (t, J=5.8 Hz, 2H), 3.22 (s, 3H), 3.91 (t, J=5.9 Hz, 2H), 4.15 (dq, J=14.1, 6.6 Hz, 1H), 4.53 (t, J=1.2 Hz, 2H), 4.94 (br d, J=6.6 Hz, 1H), 6.86 (s, 1H), 7.34 (dd, J=11.1, 7.2 Hz, 1H), 7.48-7.56 (m, 3H), 8.04-8.09 (m, 2H), 11.25 (s, 1H); LCMS (method D): Rt 2.02 min

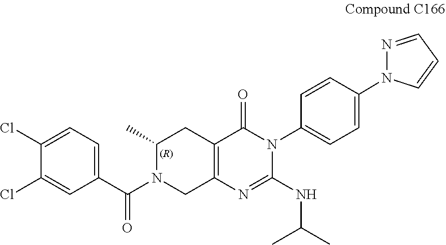

¹H NMR (400 MHz, DMSO-d₆, 100° C.) δ ppm 1.06 (d, J=6.6 Hz, 3H), 1.07 (d, J=6.6 Hz, 3H), 1.19 (d, J=6.8 Hz, 3H), 2.35 (d, J=16.3 Hz, 1H), 2.55 (br dd, J=16.5, 5.9 Hz, 1H), 4.02 (br d, J=18.7 Hz, 1H), 4.15 (dq, J=13.6, 6.8 Hz, 1H), 4.39-4.64 (m, 2H), 5.05 (br d, J=7.7 Hz, 1H), 6.55 (dd, J=2.6, 1.8 Hz, 1H), 7.33-7.38 (m, 2H), 7.41 (dd, J=8.4, 2.0 Hz, 1H), 7.66 (d, J=2.0 Hz, 1H), 7.69 (d, J=8.1 Hz, 1H), 7.75 (d, J=1.5 Hz, 1H), 7.98 (d, J=9.0 Hz, 2H), 8.47 (d, J=2.4 Hz, 1H); LCMS (method B): Rt 2.19 min

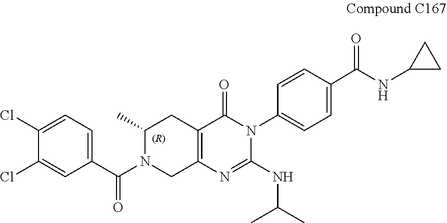

¹H NMR (400 MHz, DMSO-d₆, 81° C.) δ ppm 0.57-0.64 (m, 2H), 0.69-0.76 (m, 2H), 1.04 (d, J=6.6 Hz, 3H), 1.04 (d, J=6.6 Hz, 3H), 1.18 (d, J=6.8 Hz, 3H), 2.32 (d, J=16.5 Hz, 1H), 2.53 (br dd, J=16.4, 6.1 Hz, 1H), 2.90 (tq, J=7.4, 3.9 Hz, 1H), 4.02 (br d, J=19.2 Hz, 1H), 4.15 (dq, J=13.9, 6.7 Hz, 1H), 4.25-4.84 (m, 2H), 5.02 (br d, J=7.7 Hz, 1H), 7.31 (d, J=8.4 Hz, 2H), 7.41 (dd, J=8.3, 1.9 Hz, 1H), 7.67 (d, J=2.0 Hz, 1H), 7.70 (d, J=8.1 Hz, 1H), 7.97 (d, J=8.8 Hz, 2H), 8.33 (br d, J=4.0 Hz, 1H); LCMS (method B): Rt 2.00 min

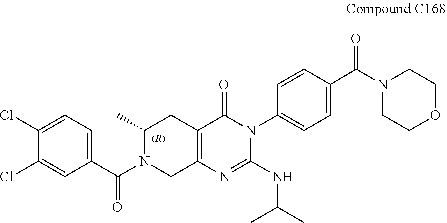

¹H NMR (400 MHz, DMSO-d₆, 81° C.) δ ppm 1.06 (d, J=6.6 Hz, 3H), 1.06 (d, J=6.6 Hz, 3H), 1.18 (d, J=6.8 Hz, 3H), 2.32 (d, J=16.3 Hz, 1H), 2.50-2.58 (m, 1H), 3.47 (m, 8H), 4.01 (br d, J=18.5 Hz, 1H), 4.13 (dq, J=13.8, 6.8 Hz, 1H), 4.23-4.81 (m, 2H), 5.09 (d, J=7.9 Hz, 1H), 7.32 (d, J=8.1 Hz, 2H), 7.41 (dd, J=8.1, 2.0 Hz, 1H), 7.52-7.60 (m, 2H), 7.66 (d, J=1.8 Hz, 1H), 7.70 (d, J=8.1 Hz, 1H); LCMS (method C): Rt 1.07 min

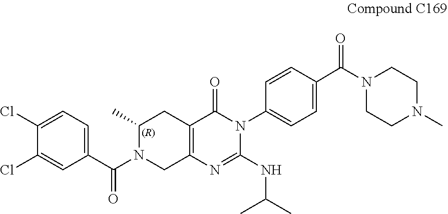

¹H NMR (400 MHz, DMSO-d₆, 81° C.) δ ppm 1.06 (d, J=6.4 Hz, 3H), 1.07 (d, J=6.4 Hz, 3H), 1.18 (d, J=6.8 Hz, 3H), 2.22 (s, 3H), 2.27-2.41 (m, 5H), 2.58 (br dd, J=16.4, 5.8 Hz, 1H), 3.45-3.59 (m, 4H), 4.02 (br d, J=17.5 Hz, 1H), 4.13 (dq, J=13.8, 6.8 Hz, 1H), 4.24-4.85 (m, 2H), 5.10 (d, J=7.7 Hz, 1H), 7.32 (d, J=8.4 Hz, 2H), 7.42 (dd, J=8.1, 2.0 Hz, 1H), 7.53 (d, J=8.6 Hz, 2H), 7.68 (d, J=1.8 Hz, 1H), 7.70 (d, J=8.1 Hz, 1H); LCMS (method B): Rt 1.95 min

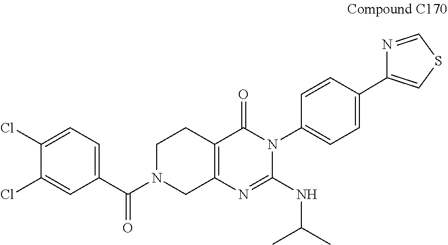 [SMILES: CC(C)Nc1nc2c(c(=O)n1-c1ccc(-c3cscn3)cc1)CCN(C(=O)c1ccc(Cl)c(Cl)c1)C2]

¹H NMR (400 MHz, DMSO-d₆, 100° C.) δ ppm 1.03-1.08 (m, 6H), 2.39-2.46 (m, 2H), 3.63-3.68 (m, 2H), 4.07-4.18 (m, 1H), 4.31 (s, 2H), 4.90 (br d, J=7.7 Hz, 1H), 7.28-7.33 (m, 2H), 7.43 (dd, J=8.1, 2.0 Hz, 1H), 7.66-7.70 (m, 2H), 8.12-8.17 (m, 3H), 9.16 (d, J=1.6 Hz, 1H); LCMS (method C): Rt 1.20 min

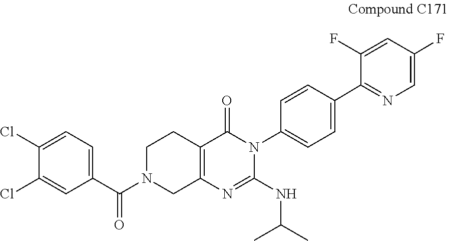 [SMILES: CC(C)Nc1nc2c(c(=O)n1-c1ccc(-c3ncc(F)cc3F)cc1)CCN(C(=O)c1ccc(Cl)c(Cl)c1)C2]

¹H NMR (400 MHz, DMSO-d₆, 100° C.) δ ppm 1.07 (d, J=6.5 Hz, 6H), 2.43 (t, J=5.9 Hz, 2H), 3.65 (br t, J=5.7 Hz, 2H), 4.13 (dq, J=13.0, 6.5 Hz, 1H), 4.32 (s, 2H), 4.96 (br d, J=4.9 Hz, 1H), 7.36-7.41 (m, 2H), 7.43 (dd, J=8.3, 1.8 Hz, 1H), 7.64-7.72 (m, 2H), 7.92 (ddd, J=11.3, 8.6, 2.4 Hz, 1H), 8.03-8.09 (m, 2H), 8.61 (d, J=2.0 Hz, 1H); LCMS (method C): Rt 1.28 min

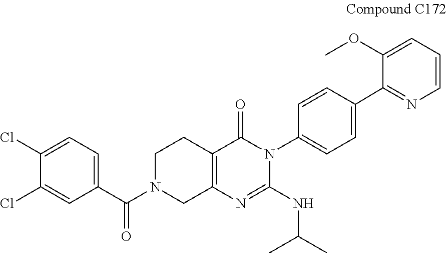 [SMILES: COc1cccnc1-c1ccc(-n2c(NC(C)C)nc3c(c2=O)CCN(C(=O)c2ccc(Cl)c(Cl)c2)C3)cc1]

¹H NMR (400 MHz, DMSO-d₆, 100° C.) δ ppm 1.03-1.09 (m, 6H), 2.39-2.46 (m, 2H), 3.60-3.72 (m, 2H), 3.90 (s, 3H), 4.06-4.17 (m, 1H), 4.28-4.36 (m, 2H), 4.86 (br s, 1H), 7.27-7.31 (m, 2H), 7.31-7.39 (m, 1H), 7.42-7.45 (m, 1H), 7.56-7.59 (m, 1H), 7.67-7.70 (m, 2H), 8.10-8.14 (m, 2H), 8.28 (dd, J=4.7, 1.4 Hz, 1H); LCMS (method C): Rt 1.22 min

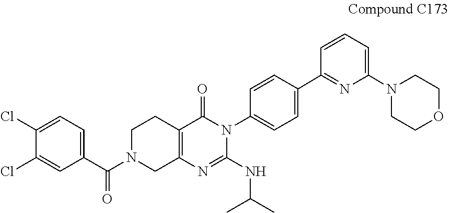 [SMILES: CC(C)Nc1nc2c(c(=O)n1-c1ccc(-c3cccc(N4CCOCC4)n3)cc1)CCN(C(=O)c1ccc(Cl)c(Cl)c1)C2]

¹H NMR (400 MHz, DMSO-d₆, 100° C.) δ ppm 1.06 (br d, J=6.1 Hz, 6H), 2.40-2.46 (m, 2H), 3.55-3.61 (m, 4H), 3.65 (br s, 2H), 3.70-3.79 (m, 4H), 4.07-4.19 (m, 1H), 4.32 (br s, 2H), 4.93 (br d, J=6.1 Hz, 1H), 6.79 (br d, J=8.5 Hz, 1H), 7.30 (br d, J=8.1 Hz, 3H), 7.43 (br d, J=8.1 Hz, 1H), 7.62-7.72 (m, 3H), 8.18 (br d, J=8.1 Hz, 2H); LCMS (method C): Rt 1.30 min

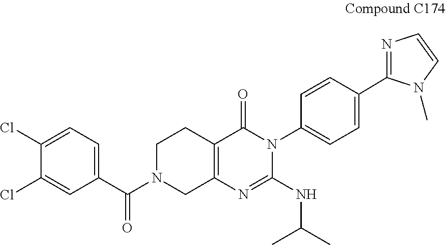 [SMILES: CC(C)Nc1nc2c(c(=O)n1-c1ccc(-c3nccn3C)cc1)CCN(C(=O)c1ccc(Cl)c(Cl)c1)C2]

¹H NMR (400 MHz, DMSO-d₆, 100° C.) δ ppm 1.07 (d, J=6.5 Hz, 6H), 2.43 (t, J=5.9 Hz, 2H), 3.65 (br t, J=5.5 Hz, 2H), 3.74 (s, 3H), 4.12 (dt, J=12.9, 6.4 Hz, 1H), 4.32 (s, 2H), 4.89 (br s, 1H), 7.14 (d, J=0.8 Hz, 1H), 7.29-7.33 (m, 2H), 7.43 (dd, J=8.1, 2.0 Hz, 1H), 7.63-7.71 (m, 5H); LCMS (method C): Rt 1.08 min

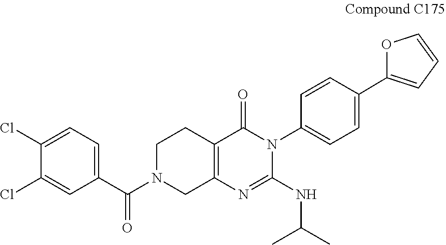 [SMILES: CC(C)Nc1nc2c(c(=O)n1-c1ccc(-c3ccco3)cc1)CCN(C(=O)c1ccc(Cl)c(Cl)c1)C2]

¹H NMR (400 MHz, DMSO-d₆, 100° C.) δ ppm 1.05 (d, J=6.5 Hz, 6H), 2.42 (t, J=5.9 Hz, 2H), 3.64 (br t, J=5.5 Hz, 2H), 4.12 (dq, J=13.4, 6.5 Hz, 1H), 4.31 (s, 2H), 4.91 (br d, J=7.7 Hz, 1H), 6.57-6.62 (m, 1H), 6.97 (dd, J=3.3, 0.8 Hz, 1H), 7.25-7.29 (m, 2H), 7.43 (dd, J=8.1, 2.0 Hz, 1H), 7.65-7.74 (m, 3H), 7.81-7.86 (m, 2H); LCMS (method C): Rt 1.29 min

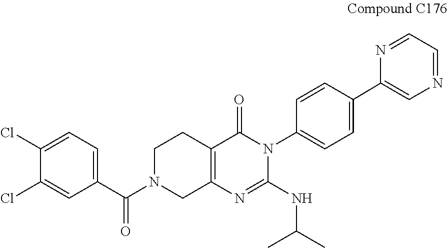 [SMILES: CC(C)Nc1nc2c(c(=O)n1-c1ccc(-c3cnccn3)cc1)CCN(C(=O)c1ccc(Cl)c(Cl)c1)C2]

¹H NMR (400 MHz, DMSO-d₆, 100° C.) δ ppm 1.06 (d, J=6.6 Hz, 6H), 2.40-2.46 (m, 2H), 3.65 (br s, 2H), 4.14 (dq, J=13.6, 6.8 Hz, 1H), 4.32 (s, 2H), 5.04 (br d, J=7.5 Hz, 1H), 7.38-7.42 (m, 2H), 7.44 (dd, J=8.1, 2.0 Hz, 1H), 7.68 (s, 1H), 7.69 (d, J=8.1 Hz, 1H), 8.28 (d, J=7.8 Hz, 2H), 8.62 (d, J=2.4 Hz, 1H), 8.73 (dd, J=2.4, 1.8 Hz, 1H), 9.27 (d, J=1.5 Hz, 1H); LCMS (method C): Rt 1.16 min

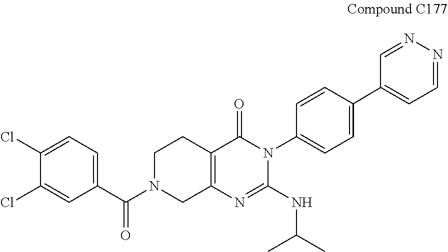 [SMILES: CC(C)Nc1nc2c(c(=O)n1-c1ccc(-c3ccnnc3)cc1)CCN(C(=O)c1ccc(Cl)c(Cl)c1)C2]

¹H NMR (400 MHz, DMSO-d₆, 100° C.) δ ppm 1.07 (d, J=6.6 Hz, 6H), 2.43 (t, J=5.7 Hz, 2H), 3.65 (br s, 2H), 4.15 (dq, J=13.5, 6.7 Hz, 1H), 4.32 (s, 2H), 5.08 (br d, J=7.3 Hz, 1H), 7.41-7.47 (m, 3H), 7.66-7.72 (m, 2H), 8.01 (dd, J=5.5, 2.6 Hz, 1H), 8.05-8.09 (m, 2H), 9.28 (dd, J=5.4, 1.2 Hz, 1H), 9.65 (dd, J=2.4, 1.1 Hz, 1H); LCMS (method C): Rt 1.07 min

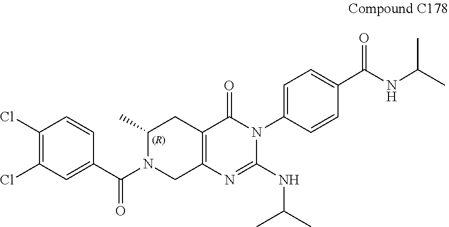

¹H NMR (400 MHz, DMSO-d₆, 81° C.) δ ppm 1.04 (d, J=6.6 Hz, 3H), 1.05 (d, J=6.6 Hz, 3H), 1.13-1.23 (m, 9H), 2.33 (d, J=16.3 Hz, 1H), 2.54 (br dd, J=16.5, 5.9 Hz, 1H), 4.02 (br d, J=18.5 Hz, 1H), 4.07-4.21 (m, 2H), 4.24-4.86 (m, 2H), 5.04 (d, J=7.7 Hz, 1H), 7.32 (br d, J=8.1 Hz, 2H), 7.42 (dd, J=8.3, 1.9 Hz, 1H), 7.68 (d, J=1.8 Hz, 1H), 7.70 (d, J=8.4 Hz, 1H), 8.00 (d, J=8.6 Hz, 2H), 8.10 (br d, J=7.9 Hz, 1H); LCMS (method D): Rt 2.07 min

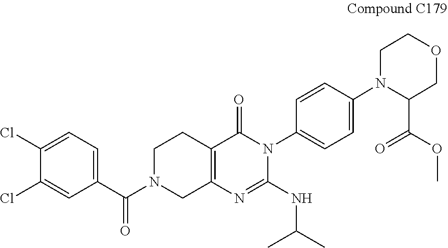 [SMILES: COC(=O)C1COCCN1c1ccc(-n2c(NC(C)C)nc3c(c2=O)CCN(C(=O)c2ccc(Cl)c(Cl)c2)C3)cc1]

¹H NMR (400 MHz, DMSO-d₆, 100° C.) δ ppm 1.00-1.08 (m, 6H), 2.40 (br t, J=5.9 Hz, 2H), 2.61-2.68 (m, 1H), 3.33-3.42 (m, 1H), 3.43-3.54 (m, 2H), 3.64 (s, 3H), 3.80 (dd, J=11.6, 3.6 Hz, 1H), 3.96 (dd, J=11.3, 3.9 Hz, 1H), 4.01-4.06 (m, 1H), 4.22-4.32 (m, 3H), 4.58 (br d, J=7.7 Hz, 1H), 4.62 (d, J=3.5 Hz, 1H), 6.96-7.12 (m, 5H), 7.43 (dd, J=8.4, 2.0 Hz, 1H), 7.66-7.71 (m, 2H); LCMS (method C): Rt 1.22 min

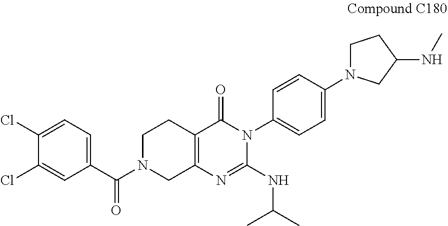 [SMILES: CNC1CCN(c2ccc(-n3c(NC(C)C)nc4c(c3=O)CCN(C(=O)c3ccc(Cl)c(Cl)c3)C4)cc2)C1]

¹H NMR (400 MHz, DMSO-d₆, 100° C.) δ ppm 1.04 (d, J=6.6 Hz, 6H), 1.78-1.87 (m, 1H), 2.07-2.18 (m, 1H), 2.35 (s, 3H), 2.40 (br t, J=5.8 Hz, 2H), 3.06 (dd, J=9.6, 4.7 Hz, 1H), 3.25-3.43 (m, 3H), 3.46 (dd, J=9.6, 6.3 Hz, 1H), 3.57-3.67 (m, 2H), 4.07 (dq, J=13.7, 6.7 Hz, 1H), 4.28 (s, 2H), 4.65 (br d, J=7.7 Hz, 1H), 6.60-6.66 (m, 2H), 6.92-6.99 (m, 2H), 7.42 (dd, J=8.1, 2.0 Hz, 1H), 7.66-7.72 (m, 2H); LCMS (method D): Rt 1.84 min

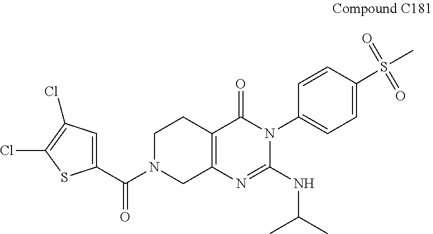 [SMILES: CC(C)Nc1nc2c(c(=O)n1-c1ccc(S(C)(=O)=O)cc1)CCN(C(=O)c1cc(Cl)c(Cl)s1)C2]

¹H NMR (400 MHz, DMSO-d₆, 150° C.) δ ppm 1.09 (d, J=6.4 Hz, 6H), 2.48-2.51 (m, 2H), 3.22 (s, 3H), 3.83 (t, J=5.9 Hz, 2H), 4.16 (dq, J=13.7, 6.7 Hz, 1H), 4.44 (t, J=1.2 Hz, 2H), 4.98 (br d, J=7.0 Hz, 1H), 7.46 (s, 1H), 7.49-7.52 (m, 2H), 8.04-8.10 (m, 2H); LCMS (method B): Rt 2.07 min

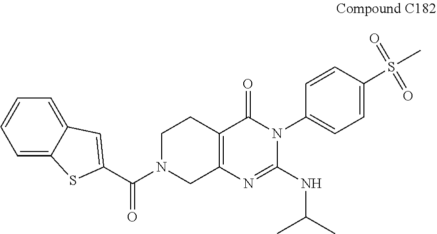 [SMILES: CC(C)Nc1nc2c(c(=O)n1-c1ccc(S(C)(=O)=O)cc1)CCN(C(=O)c1cc3ccccc3s1)C2]

¹H NMR (400 MHz, DMSO-d₆, 150° C.) δ ppm 1.08 (d, J=6.6 Hz, 6H), 2.51 (t, J=5.9 Hz, 2H), 3.22 (s, 3H), 3.87 (t, J=5.9 Hz, 2H), 4.14 (dq, J=13.5, 6.5 Hz, 1H), 4.49 (t, J=1.2 Hz, 2H), 4.95 (br d, J=6.4 Hz, 1H), 7.38-7.46 (m, 2H), 7.50-7.55 (m, 2H), 7.7 (d, J=0.8 Hz, 1H), 7.90-7.97 (m, 2H), 8.04-8.10 (m, 2H); LCMS (method B): Rt 1.94 min

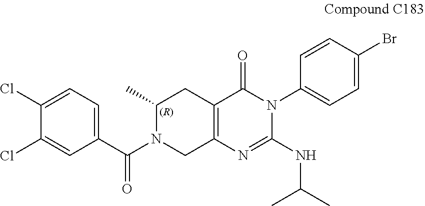

¹H NMR (400 MHz, DMSO-d₆, 81° C.) δ ppm 1.05 (d, J=6.6 Hz, 3H), 1.06 (d, J=6.6 Hz, 3H), 1.17 (d, J=6.8 Hz, 3H), 2.31 (br d, J=16.3 Hz, 1H), 2.50-2.57 (m, 1H), 4.00 (br d, J=18.9 Hz, 1H), 4.15 (dq, J=13.7, 6.8 Hz, 1H), 4.23-4.87 (m, 2H), 5.25 (br d, J=7.7 Hz, 1H), 7.21 (br d, J=8.6 Hz, 2H), 7.41 (dd, J=8.4, 2.0 Hz, 1H), 7.67 (d, J=2.0 Hz, 1H), 7.68-7.76 (m, 3H); LCMS (method C): 1.24 min

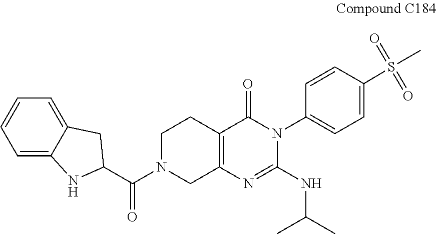 [SMILES: CC(C)Nc1nc2c(c(=O)n1-c1ccc(S(C)(=O)=O)cc1)CCN(C(=O)C1Cc3ccccc3N1)C2]

¹H NMR (400 MHz, DMSO-d₆, 101° C.) δ ppm 1.08 (d, J=6.6 Hz, 6H), 2.43 (br s, 2H), 3.15 (dd, J=15.9, 6.3 Hz, 1H), 3.25 (s, 3H), 3.35 (dd, J=15.9, 10.7 Hz, 1H), 3.72 (t, J=5.8 Hz, 2H), 4.18 (dq, J=13.2, 6.5 Hz, 1H), 4.33 (br s, 2H), 4.72 (dd, J=10.6, 6.2 Hz, 1H), 5.22 (br d, J=7.7 Hz, 1H), 5.41 (br s, 1H), 6.53-6.61 (m, 2H), 6.92 (t, J=7.3 Hz, 1H), 6.99 (d, J=7.3 Hz, 1H), 7.50-7.56 (m, 2H), 8.05-8.10 (m, 2H); LCMS (method D): Rt 1.66 min

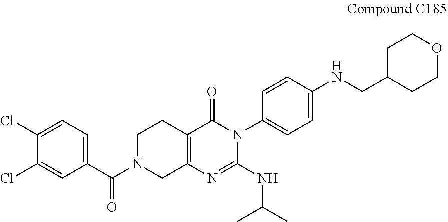 [SMILES: CC(C)Nc1nc2c(c(=O)n1-c1ccc(NCC3CCOCC3)cc1)CCN(C(=O)c1ccc(Cl)c(Cl)c1)C2]

20 ¹H NMR (400 MHz, DMSO-d₆, 100° C.) δ ppm 1.04 (d, J=6.6 Hz, 6H), 1.20-1.33 (m, 2H), 1.65-1.74 (m, 2H), 1.77-1.88 (m, 1H), 2.40 (t, J=5.8 Hz, 2H), 2.98 (t, J=6.2 Hz, 2H), 3.31 (td, J=11.5, 2.3 Hz, 2H), 3.53-3.72 (m, 2H), 3.83-3.89 (m, 2H), 4.06 (dq, J=13.7, 6.7 Hz, 1H), 4.28 (br s, 2H), 4.65 (br d, J=7.7 Hz, 1H), 5.72 (t, J=5.6 Hz, 1H), 6.68-6.73 (m, 2H), 6.84-6.89 (m, 2H), 7.42 (dd, J=8.1, 2.0 Hz, 1H), 7.66-7.70 (m, 2H); LCMS (method B): Rt 2.20 min

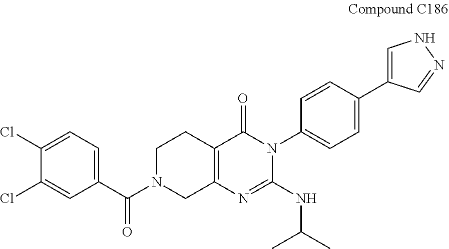 [SMILES: CC(C)Nc1nc2c(c(=O)n1-c1ccc(-c3cn[nH]c3)cc1)CCN(C(=O)c1ccc(Cl)c(Cl)c1)C2]

¹H NMR (400 MHz, DMSO-d₆, 100° C.) δ ppm 1.05 (d, J=6.5 Hz, 6H), 2.42 (t, J=5.9 Hz, 2H), 3.60-3.68 (m, 2H), 4.06-4.17 (m, 1H), 4.31 (s, 2H), 4.76-4.88 (m, 1H), 7.16-7.22 (m, 2H), 7.43 (dd, J=8.3, 1.8 Hz, 1H), 7.66-7.71 (m, 2H), 7.71-7.75 (m, 2H), 8.02 (s, 2H); LCMS (method C): Rt 1.05 min

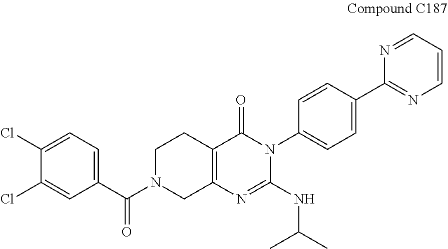 [SMILES: CC(C)Nc1nc2c(c(=O)n1-c1ccc(-c3ncccn3)cc1)CCN(C(=O)c1ccc(Cl)c(Cl)c1)C2]

¹H NMR (400 MHz, DMSO-d₆, 100° C.) δ ppm 1.06 (d, J=6.6 Hz, 6H), 2.43 (t, J=5.8 Hz, 2H), 3.59-3.71 (m, 2H), 4.14 (dq, J=13.6, 6.7 Hz, 1H), 4.32 (br s, 2H), 5.02 (br d, J=7.7 Hz, 1H), 7.36-7.40 (m, 2H), 7.42-7.47 (m, 2H), 7.66-7.72 (m, 2H), 8.50-8.57 (m, 2H), 8.92 (d, J=4.8 Hz, 2H); LCMS (method C): Rt 1.17 min

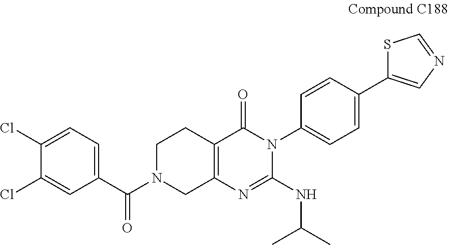 [SMILES: CC(C)Nc1nc2c(c(=O)n1-c1ccc(-c3cncs3)cc1)CCN(C(=O)c1ccc(Cl)c(Cl)c1)C2]

¹H NMR (400 MHz, DMSO-d₆, 100° C.) δ ppm 1.06 (d, J=6.6 Hz, 6H), 2.42 (t, J=5.8 Hz, 2H), 3.64 (br t, J=4.1 Hz, 2H), 4.14 (br dd, J=13.6, 6.8 Hz, 1H), 4.31 (s, 2H), 5.07 (br d, J=7.0 Hz, 1H), 7.28-7.34 (m, 2H), 7.43 (dd, J=8.4, 2.0 Hz, 1H), 7.65-7.71 (m, 2H), 7.80-7.85 (m, 2H), 8.33 (s, 1H), 9.07 (s, 1H); LCMS (method C): Rt 1.14 min

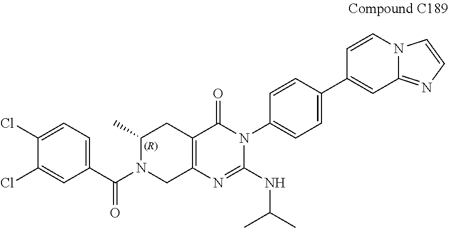

¹H NMR (400 MHz, DMSO-d₆, 100° C.) δ ppm 1.07 (d, J=6.6, 2.9 Hz, 3H), 1.08 (d, J=6.6 Hz, 3H), 1.20 (d, J=6.8 Hz, 3H), 2.35 (d, J=16.3 Hz, 1H), 2.56 (br dd, J=16.4, 5.8 Hz, 1H), 4.03 (br d, J=18.7 Hz, 1H), 4.15 (dq, J=13.5, 6.8 Hz, 1H), 4.41-4.67 (m, 2H), 5.02 (br d, J=7.7 Hz, 1H), 7.29 (dd, J=7.3, 1.8 Hz, 1H), 7.36 (d, J=8.6 Hz, 2H), 7.42 (dd, J=8.3, 1.9 Hz, 1H), 7.60 (d, J=0.9 Hz, 1H), 7.67 (d, J=2.0 Hz, 1H), 7.69 (d, J=8.1 Hz, 1H), 7.89-7.94 (m, 2H), 7.96 (d, J=8.6 Hz, 2H), 8.59 (dd, J=7.2, 0.8 Hz, 1H); LCMS (method C): Rt 1.12 min

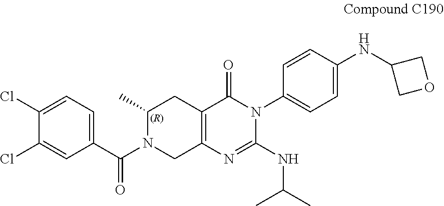

¹H NMR (400 MHz, DMSO-d₆, 100° C.) δ ppm 1.04 (d, J=6.4 Hz, 3H), 1.05 (d, J=6.4 Hz, 3H), 1.17 (d, J=6.6 Hz, 3H), 2.31 (d, J=16.5 Hz, 1H), 2.50-2.56 (m, 1H), 3.98 (br d, J=18.9 Hz, 1H), 4.06 (dq, J=13.7, 6.6 Hz, 1H), 4.46 (t, J=6.1 Hz, 1H), 4.40-4.62 (m, 3H), 4.58 (dq, J=12.8, 6.4 Hz, 1H), 4.66 (br d, J=7.7 Hz, 1H), 4.86 (t, J=6.5 Hz, 2H), 6.42 (d, J=6.4 Hz, 1H), 6.61 (d, J=8.8 Hz, 2H), 6.92 (br d, J=8.6 Hz, 2H), 7.40 (dd, J=8.1, 2.0 Hz, 1H), 7.65 (d, J=2.0 Hz, 1H), 7.68 (d, J=8.1 Hz, 1H); LCMS (method C): Rt 1.16 min

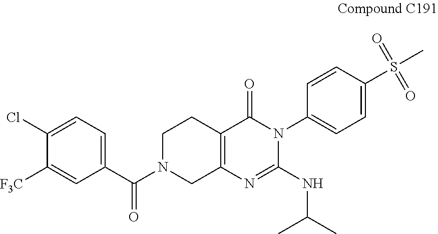 [SMILES: CC(C)Nc1nc2c(c(=O)n1-c1ccc(S(C)(=O)=O)cc1)CCN(C(=O)c1ccc(Cl)c(C(F)(F)F)c1)C2]

¹H NMR (400 MHz, DMSO-d₆, 126° C.) δ ppm 1.07 (d, J=6.6 Hz, 6H), 2.43 (t, J=5.8 Hz, 2H), 3.23 (s, 3H), 3.65 (t, J=5.8 Hz, 2H), 4.14 (dq, J=13.6, 6.6 Hz, 1H), 4.33 (s, 2H), 5.08 (br d, J=7.7 Hz, 1H), 7.49-7.54 (m, 2H), 7.72-7.79 (m, 2H), 7.84 (d, J=1.8 Hz, 1H), 8.05-8.10 (m, 2H); LCMS (method B): Rt 2.02 min

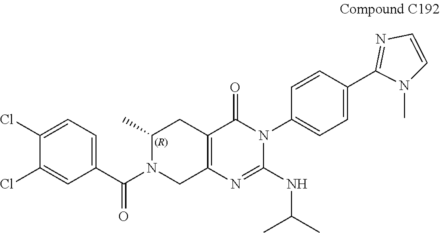

¹H NMR (400 MHz, DMSO-d₆, 81° C.) δ ppm 1.07 (d, J=6.5 Hz, 3H), 1.08 (d, J=6.5 Hz, 3H), 1.19 (d, J=6.8 Hz, 3H), 2.33 (d, J=16.3 Hz, 1H), 2.55 (br dd, J=16.3, 6.2 Hz, 1H), 3.75 (s, 3H), 4.02 (br d, J=18.7 Hz, 1H), 4.14 (dq, J=13.9, 6.7 Hz, 1H), 4.23-4.79 (m, 2H), 5.04 (d, J=7.7 Hz, 1H), 7.15 (d, J=1.1 Hz, 1H), 7.32 (d, J=8.4 Hz, 2H), 7.42 (dd, J=8.1, 2.0 Hz, 1H), 7.62-7.74 (m, 5H); LCMS (method C): Rt 1.11 min

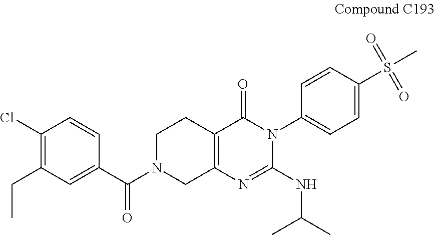 [SMILES: CCc1cc(C(=O)N2CCc3c(nc(NC(C)C)n(-c4ccc(S(C)(=O)=O)cc4)c3=O)C2)ccc1Cl]

¹H NMR (400 MHz, DMSO-d₆, 150° C.) δ ppm 1.07 (d, J=6.4 Hz, 6H), 1.22 (t, J=7.5 Hz, 3H), 2.42 (t, J=5.8 Hz, 2H), 2.78 (q, J=7.5 Hz, 2H), 3.22 (s, 3H), 3.64 (t, J=5.9 Hz, 2H), 4.12 (dq, J=13.6, 6.6 Hz, 1H), 4.31 (t, J=1.3 Hz, 2H), 4.92 (br d, J=7.3 Hz, 1H), 7.26 (dd, J=8.1, 2.0 Hz, 1H), 7.37 (d, J=2.0 Hz, 1H), 7.44 (d, J=8.1 Hz, 1H), 7.49-7.54 (m, 2H), 8.04-8.09 (m, 2H); LCMS (method B): Rt 2.06 min

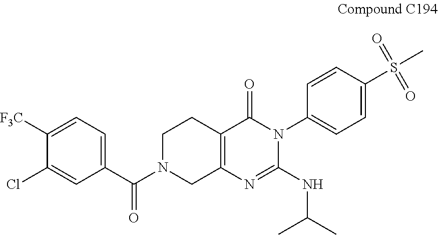 [SMILES: CC(C)Nc1nc2c(c(=O)n1-c1ccc(S(C)(=O)=O)cc1)CCN(C(=O)c1ccc(C(F)(F)F)c(Cl)c1)C2]

¹H NMR (400 MHz, DMSO-d₆, 126° C.) δ ppm 1.07 (d, J=6.6 Hz, 6H), 2.43 (br t, J=5.8 Hz, 2H), 3.23 (s, 3H), 3.65 (br t, J=5.3 Hz, 2H), 4.14 (dq, J=13.8, 6.8 Hz, 1H), 4.32 (s, 2H), 5.10 (br d, J=7.3 Hz, 1H), 7.49-7.54 (m, 2H), 7.59 (d, J=8.1 Hz, 1H), 7.73 (s, 1H), 7.90 (d, J=8.1 Hz, 1H), 8.04-8.10 (m, 2H); LCMS (method B): Rt 2.03 min

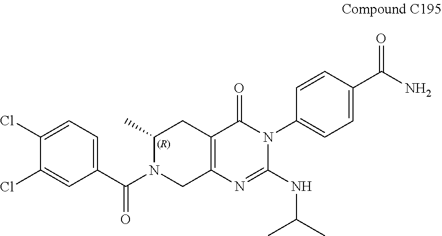

¹H NMR (400 MHz, CDCl₃, 56° C.) δ ppm 1.09 (d, J=6.4 Hz, 6H), 1.25 (d, J=7.0 Hz, 3H), 2.51 (d, J=16.7 Hz, 1H), 2.67 (br dd, J=16.7, 5.9 Hz, 1H), 3.74 (br d, J=7.5 Hz, 1H), 4.03 (br d, J=19.2 Hz, 1H), 4.16 (dq, J=13.4, 6.6 Hz, 1H), 4.25-5.18 (m, 2H), 5.55-6.32 (m, 2H), 7.22-7.28 (m, 1H), 7.28-7.41 (m, 2H), 7.48-7.55 (m, 2H), 7.93-8.05 (m, 2H); LCMS (method C): Rt 1.02 min

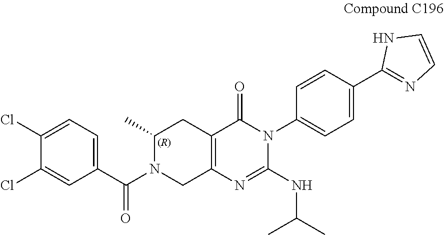

¹H NMR (400 MHz, CDCl₃, 56° C.) δ ppm 1.06 (br d, J=6.2 Hz, 6H), 1.21-1.38 (m, 3H), 2.56 (br d, J=16.5 Hz, 1H), 2.68-2.81 (m, 1H), 3.86-3.97 (m, 1H), 4.01-4.22 (m, 2H), 4.33-5.20 (m, 2H), 6.90-7.91 (m, 9H), 10.99 (s, 1H); LCMS (method C): Rt 1.06 min

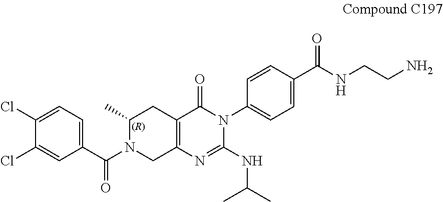

¹H NMR (400 MHz, DMSO-d₆, 81° C.) δ ppm 1.01-1.07 (m, 6H), 1.18 (d, J=6.8 Hz, 3H), 2.33 (d, J=16.1 Hz, 1H), 2.50-2.59 (m, 1H), 2.75 (t, J=6.4 Hz, 2H), 3.32 (br t, J=5.2 Hz, 2H), 3.51-3.64 (m, 1H), 3.94-4.08 (m, 1H), 4.14 (dq, J=13.8, 6.8 Hz, 1H), 4.24-4.86 (br s, 2H), 5.04 (br d, J=7.9 Hz, 1H), 7.33 (br d, J=8.1 Hz, 2H), 7.42 (dd, J=8.3, 1.9 Hz, 1H), 7.68 (d, J=2.0 Hz, 1H), 7.70 (d, J=8.4 Hz, 1H), 7.97-8.03 (m, 2H), 8.26-8.33 (br s, 1H); LCMS (method C): Rt 0.94 min

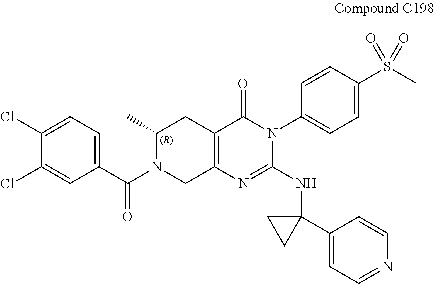

¹H NMR (400 MHz, CDCl₃, 56° C.) δ ppm 1.09-1.43 (m, 7H), 2.42-2.55 (m, 1H), 2.59-2.73 (m, 1H), 3.11 (s, 3H), 3.92 (br d, J=19.4 Hz, 1H), 4.20-5.12 (m, 3H), 6.68-7.23 (m, 3H), 7.40-7.61 (m, 4H), 8.18 (d, J=8.3 Hz, 2H), 8.27-8.88 (m, 2H); LCMS (method C): Rt 0.97 min

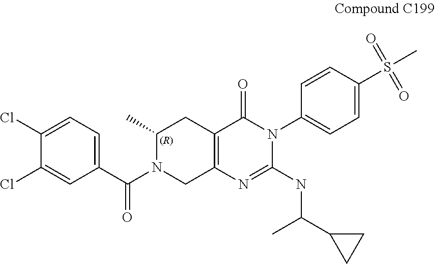

¹H NMR (400 MHz, DMSO-d₆, 81° C.) δ ppm 0.11-0.19 (m, 1H), 0.20-0.44 (m, 3H), 0.85-0.98 (m, 1H), 1.10-1.15 (m, 3H), 1.18 (dd, J=6.8, 1.3 Hz, 3H), 2.32 (br d, J=16.1 Hz, 1H), 2.50-2.58 (m, 1H), 3.27 (s, 3H), 3.37-3.63 (m, 1H), 3.99 (br d, J=18.1 Hz, 1H), 4.21-4.78 (m, 2H), 5.44 (br dd, J=7.7, 2.4 Hz, 1H), 7.41 (dd, J=8.3, 1.9 Hz, 1H), 7.56 (br d, J=8.4 Hz, 2H), 7.67 (d, J=1.8 Hz, 1H), 7.70 (d, J=8.1 Hz, 1H), 8.09 (d, J=8.8 Hz, 2H); LCMS (method C): Rt 1.15 min

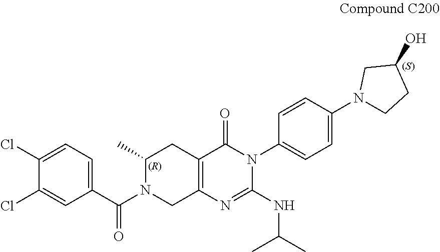

¹H NMR (400 MHz, DMSO-d₆, 100° C.) δ ppm 1.04 (d, J=6.4 Hz, 3H), 1.05 (d, J=6.4 Hz, 3H), 1.18 (d, J=6.8 Hz, 3H), 1.89-1.98 (m, 1H), 2.04-2.13 (m, 1H), 2.33 (d, J=16.3 Hz, 1H), 2.52 (br dd, J=16.5, 6.2 Hz, 1H), 3.14 (dd, J=10.1, 2.2 Hz, 1H), 3.32 (td, J=8.8, 4.2 Hz, 1H), 3.36-3.43 (m, 1H), 3.47 (dd, J=10.1, 5.1 Hz, 1H), 3.99 (br d, J=18.5 Hz, 1H), 4.07 (dq, J=13.5, 6.7 Hz, 1H), 4.36-4.60 (m, 3H), 4.62-4.69 (m, 2H), 6.63 (d, J=9.0 Hz, 2H), 6.97 (br d, J=8.6 Hz, 2H), 7.40 (dd, J=8.1, 2.0 Hz, 1H), 7.65 (d, J=2.0 Hz, 1H), 7.68 (d, J=8.1 Hz, 1H); LCMS (method D): Rt 2.08 min

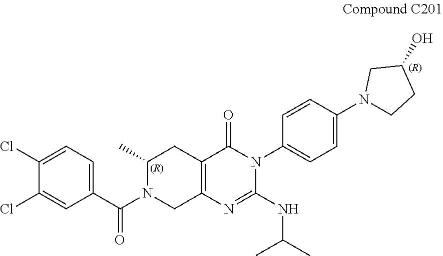

¹H NMR (400 MHz, DMSO-d₆, 100° C.) δ ppm 1.04 (d, J=6.4 Hz, 3H), 1.05 (d, J=6.4 Hz, 3H), 1.18 (d, J=6.8 Hz, 3H), 1.89-1.98 (m, 1H), 2.02-2.14 (m, 1H), 2.33 (d, J=16.5 Hz, 1H), 2.49-2.58 (m, 1H), 3.14 (dd, J=10.1, 2.4 Hz, 1H), 3.32 (td, J=8.8, 4.1 Hz, 1H), 3.37-3.43 (m, 1H), 3.47 (dd, J=10.1, 5.1 Hz, 1H), 3.99 (br d, J=19.2 Hz, 1H), 4.07 (dq, J=13.7, 6.6 Hz, 1H), 4.37-4.62 (m, 3H), 4.61-4.67 (m, 2H), 6.63 (d, J=9.0 Hz, 2H), 6.97 (br d, J=8.8 Hz, 2H), 7.40 (dd, J=8.3, 1.9 Hz, 1H), 7.65 (d, J=1.8 Hz, 1H), 7.68 (d, J=8.1 Hz, 1H); LCMS (method D): Rt 2.03 min

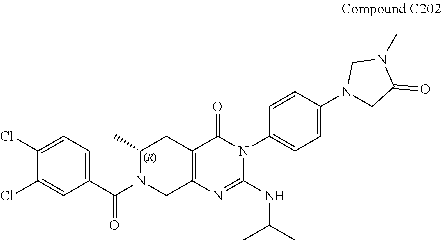

¹H NMR (400 MHz, DMSO-d₆, 100° C.) δ ppm 1.04 (d, J=6.5 Hz, 3H), 1.05 (d, J=6.5 Hz, 3H), 1.18 (d, J=6.8 Hz, 3H), 2.33 (d, J=16.3 Hz, 1H), 2.50-2.57 (m, 1H), 2.90 (s, 3H), 3.88 (s, 2H), 4.00 (br d, J=18.9 Hz, 1H), 4.08 (dq, J=13.4, 6.6 Hz, 1H), 4.35-4.61 (m, 2H), 4.68 (br d, J=7.7 Hz, 1H), 4.78 (s, 2H), 6.71 (d, J=9.0 Hz, 2H), 7.09 (br d, J=8.6 Hz, 2H), 7.40 (dd, J=8.1, 2.0 Hz, 1H), 7.65 (d, J=2.0 Hz, 1H), 7.69 (d, J=8.4 Hz, 1H); LCMS (method D): Rt 1.95 min

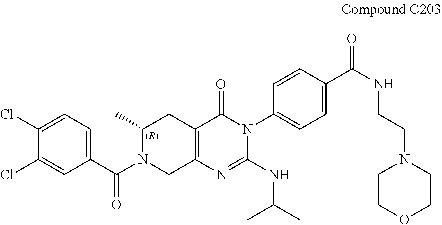

¹H NMR (400 MHz, DMSO-d₆, 81° C.) δ ppm 1.04 (d, J=6.6 Hz, 3H), 1.05 (d, J=6.6 Hz, 3H), 1.18 (d, J=6.8 Hz, 3H), 2.32 (br d, J=16.3 Hz, 1H), 2.40-2.47 (m, 4H), 2.49-2.57 (m, 1H), 2.52 (t, J=6.8 Hz, 2H), 3.43 (q, J=6.6 Hz, 2H), 3.54-3.61 (m, 4H), 4.02 (br d, J=17.8 Hz, 1H), 4.14 (dq, J=13.9, 6.7 Hz, 1H), 4.26-4.80 (m, 2H), 5.08 (br d, J=7.9 Hz, 1H), 7.33 (d, J=8.4 Hz, 2H), 7.42 (dd, J=8.1, 2.0 Hz, 1H), 7.68 (d, J=2.0 Hz, 1H), 7.70 (d, J=8.4 Hz, 1H), 7.98 (d, J=8.6 Hz, 2H), 8.27 (br t, J=5.3 Hz, 1H); LCMS (method C): Rt 1.06 min

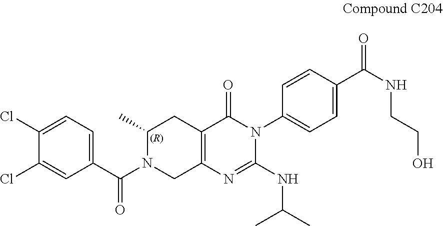

¹H NMR (400 MHz, DMSO-d₆, 81° C.) δ ppm 0.99-1.07 (m, 6H), 1.18 (d, J=6.8 Hz, 3H), 2.33 (d, J=16.1 Hz, 1H), 2.50-2.59 (m, 1H), 3.39 (q, J=6.0 Hz, 2H), 3.51-3.58 (m, 3H), 4.02 (br d, J=17.8 Hz, 1H), 4.14 (dq, J=13.6, 6.7 Hz, 1H), 4.28-4.71 (m, 1H), 5.03 (d, J=7.9 Hz, 1H), 7.33 (br d, J=8.1 Hz, 2H), 7.42 (dd, J=8.3, 1.9 Hz, 1H), 7.67 (d, J=2.0 Hz, 1H), 7.70 (d, J=8.4 Hz, 1H), 8.00 (d, J=8.6 Hz, 2H), 8.27 (br t, J=5.7 Hz, 1H); LCMS (method B): Rt 1.81 min

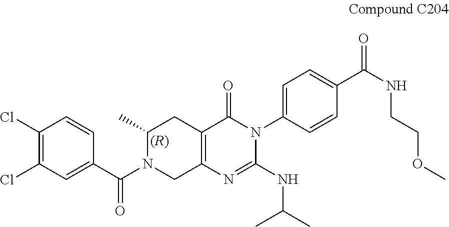

¹H NMR (400 MHz, DMSO-d₆, 81° C.) δ ppm 0.98-1.09 (m, 6H), 1.18 (br d, J=6.5 Hz, 3H), 2.33 (br d, J=16.2 Hz, 1H), 2.50-2.60 (m, 1H), 3.29 (s, 3H), 3.40-3.57 (m, 4H), 4.02 (br d, J=18.7 Hz, 1H), 4.07-4.21 (m, 1H), 4.26-4.80 (m, 2H), 5.00-5.08 (m, 1H), 7.33 (br d, J=7.7 Hz, 2H), 7.41 (dd, J=8.1, 1.6 Hz, 1H), 7.65-7.72 (m, 2H), 8.00 (br d, J=8.5 Hz, 2H), 8.35 (br s, 1H); LCMS (method C): Rt 1.08 min

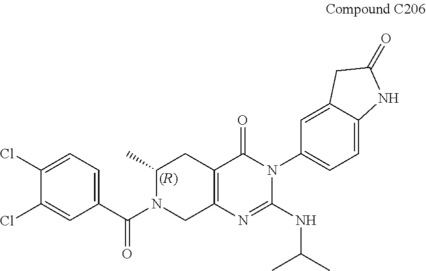

¹H NMR (400 MHz, DMSO-d₆, 100° C.) δ ppm 1.05 (d, J=6.5 Hz, 3H), 1.06 (d, J=6.5 Hz, 3H), 1.18 (d, J=6.8 Hz, 3H), 2.32 (d, J=16.3 Hz, 1H), 2.53 (br dd, J=16.4, 5.8 Hz, 1H), 3.50 (br s, 2H), 4.00 (br d, J=18.7 Hz, 1H), 4.11 (dq, J=13.5, 6.7 Hz, 1H), 4.30-4.68 (m, 2H), 4.86 (br d, J=7.7 Hz, 1H), 6.92-6.96 (m, 1H), 6.97-7.08 (m, 2H), 7.40 (dd, J=8.3, 1.9 Hz, 1H), 7.65 (d, J=2.0 Hz, 1H), 7.68 (d, J=8.1 Hz, 1H), 10.26 (br s, 1H); LCMS (method D): Rt 1.85 min

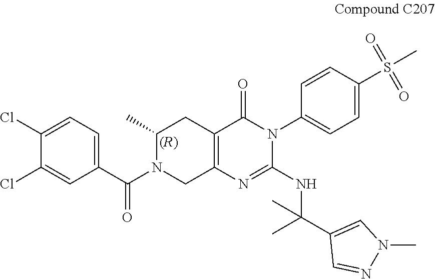

¹H NMR (400 MHz, DMSO-d₆, 81° C.) δ ppm 1.16 (d, J=6.8 Hz, 3H), 1.60 (s, 3H), 1.64 (s, 3H), 2.31 (d, J=16.4 Hz, 1H), 2.50-2.59 (m, 1H), 3.26 (s, 3H), 3.71 (s, 3H), 3.95 (br d, J=19.4 Hz, 1H), 4.22-4.74 (m, 2H), 4.63 (s, 1H), 7.23 (s, 1H), 7.40 (dd, J=8.3, 1.9 Hz, 1H), 7.45 (s, 1H), 7.62 (d, J=8.4 Hz, 2H), 7.67 (d, J=2.0 Hz, 1H), 7.70 (d, J=8.4 Hz, 1H), 8.10 (d, J=8.8 Hz, 2H); LCMS (method D): Rt 1.85 min

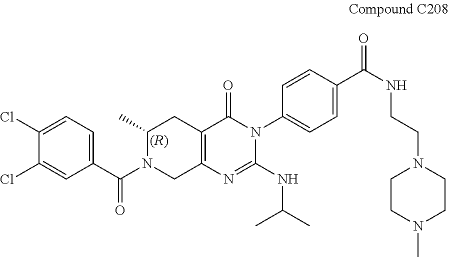

¹H NMR (400 MHz, DMSO-d₆, 81° C.) δ ppm 1.04 (d, J=6.4 Hz, 3H), 1.05 (d, J=6.4 Hz, 3H), 1.18 (d, J=6.8 Hz, 3H), 2.15 (s, 3H), 2.29-2.36 (m, 5H), 2.43-2.58 (m, 7H), 3.41 (q, J=6.6 Hz, 2H), 4.02 (br d, J=17.4 Hz, 1H), 4.14 (dq, J=13.8, 6.7 Hz, 1H), 4.25-4.75 (m, 2H), 5.09 (d, J=7.7 Hz, 1H), 7.33 (br d, J=8.4 Hz, 2H), 7.42 (dd, J=8.3, 1.9 Hz, 1H), 7.68 (d, J=2.0 Hz, 1H), 7.70 (d, J=8.1 Hz, 1H), 7.97 (d, J=8.8 Hz, 2H), 8.25 (br t, J=5.5 Hz, 1H); LCMS (method C): Rt 1.01 min

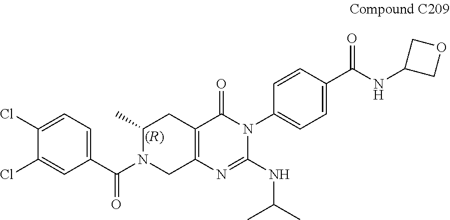

¹H NMR (400 MHz, DMSO-d₆, 81° C.) δ ppm 1.04 (d, J=6.4 Hz, 3H), 1.05 (d, J=6.4 Hz, 3H), 1.18 (d, J=6.8 Hz, 3H), 2.33 (br d, J=16.3 Hz, 1H), 2.50-2.60 (m, 1H), 4.02 (br d, J=18.9 Hz, 1H), 4.14 (dq, J=13.6, 7.0 Hz, 1H), 4.35-4.65 (m, 2H), 4.62 (t, J=6.5 Hz, 2H), 4.79 (t, J=6.8 Hz, 2H), 4.98-5.04 (m, 1H), 5.04-5.09 (m, 1H), 7.36 (br d, J=8.4 Hz, 2H), 7.42 (dd, J=8.1, 2.0 Hz, 1H), 7.68 (d, J=1.8 Hz, 1H), 7.70 (d, J=8.4 Hz, 1H), 8.04 (d, J=8.5 Hz, 2H), 9.00 (br d, J=6.2 Hz, 1H); LCMS (method C): Rt 1.07 min

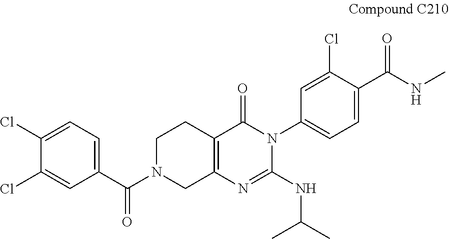 [SMILES: CNC(=O)c1ccc(-n2c(NC(C)C)nc3c(c2=O)CCN(C(=O)c2ccc(Cl)c(Cl)c2)C3)cc1Cl]

¹H NMR (400 MHz, DMSO-d₆, 100° C.) δ ppm 1.07 (d, J=6.6 Hz, 6H), 2.40 (t, J=5.9 Hz, 2H), 2.80 (d, J=4.4 Hz, 3H), 3.57-3.70 (m, 2H), 4.08-4.20 (m, 1H), 4.30 (s, 2H), 5.26 (br s, 1H), 7.25 (dd, J=8.1, 2.0 Hz, 1H), 7.39 (d, J=1.8 Hz, 1H), 7.42 (dd, J=8.1, 2.0 Hz, 1H), 7.57 (d, J=8.1 Hz, 1H), 7.67 (d, J=1.8 Hz, 1H), 7.69 (d, J=8.1 Hz, 1H), 8.07 (br s, 1H); LCMS (method C): Rt 1.01 min

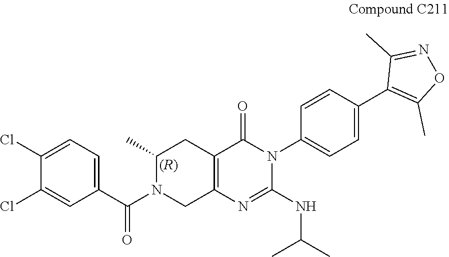

¹H NMR (400 MHz, DMSO-d₆, 100° C.) δ ppm 1.07 (d, J=6.6 Hz, 3H), 1.08 (d, J=6.6 Hz, 3H), 1.19 (d, J=6.8 Hz, 3H), 2.28 (s, 3H), 2.35 (d, J=16.1 Hz, 1H), 2.45 (s, 3H), 2.55 (br dd, J=16.4, 5.8 Hz, 1H), 4.02 (br d, J=18.9 Hz, 1H), 4.12 (sext, J=13.2, 6.6 Hz, 1H),4.31-4.71 (m, 2H), 4.82 (br d, J=7.5 Hz, 1H), 7.33-7.38 (m, 2H), 7.41 (dd, J=8.1, 2.0 Hz, 1H), 7.54 (d, J=8.6 Hz, 2H), 7.66 (d, J=2.0 Hz, 1H), 7.69 (d, J=8.1 Hz, 1H); LCMS (method C): Rt 1.26 min

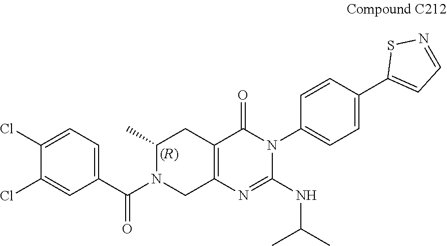

¹H NMR (400 MHz, CDCl₃, 47° C.) δ ppm 1.12 (d, J=6.4 Hz, 6H), 1.26 (d, J=6.8 Hz, 3H), 2.52 (d, J=16.7 Hz, 1H), 2.69 (br dd, J=16.4, 5.4 Hz, 1H), 3.85 (br d, J=7.5 Hz, 1H), 4.03 (br d, J=19.1 Hz, 1H), 4.18 (dq, J=13.1, 6.6 Hz, 1H), 4.46 (br s, 2H), 7.25-7.29 (m, 1H), 7.29-7.38 (m, 2H), 7.46 (d, J=1.8 Hz, 1H), 7.52 (d, J=8.1 Hz, 1H), 7.53 (d, J=2.0 Hz, 1H), 7.80 (d, J=7.7 Hz, 2H), 8.51 (d, J=1.8 Hz, 1H); LCMS (method C): Rt 1.19 min

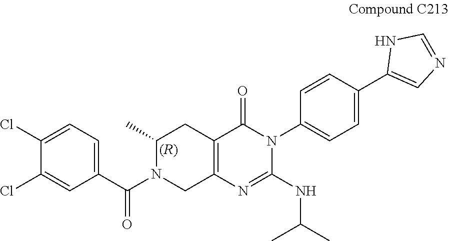

¹H NMR (400 MHz, DMSO-d₆, 100° C.) δ ppm 1.05 (d, J=6.6 Hz, 3H), 1.06 (d, J=6.6 Hz, 3H), 1.19 (d, J=6.8 Hz, 3H), 2.34 (br d, J=16.3 Hz, 1H), 2.55 (br dd, J=16.4, 5.8 Hz, 1H), 4.02 (br d, J=18.3 Hz, 1H), 4.12 (dq, J=13.8, 6.7 Hz, 1H), 4.40-4.62 (m, 2H), 4.90 (br d, J=7.5 Hz, 1H), 7.26 (d, J=8.6 Hz, 2H), 7.41 (dd, J=8.3, 1.9 Hz, 1H), 7.66 (d, J=1.8 Hz, 1H), 7.69 (d, J=8.4 Hz, 1H), 7.75 (s, 1H), 7.96 (d, J=8.6 Hz, 2H), 8.11 (s, 1H); LCMS (method C): Rt 1.01 min

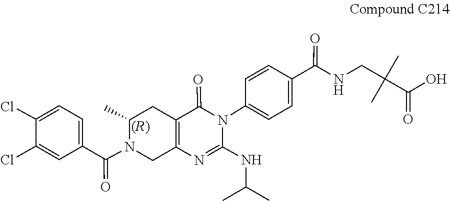

¹H NMR (400 MHz, DMSO-d₆, 81° C.) δ ppm 1.04 (d, J=6.4 Hz, 3H), 1.05 (d, J=6.4 Hz, 3H), 1.15 (s, 6H), 1.18 (d, J=6.8 Hz, 3H), 2.32 (br d, J=16.5 Hz, 1H), 2.50-2.58 (m, 1H), 3.45 (br d, J=2.2 Hz, 2H), 4.02 (br d, J=19.2 Hz, 1H), 4.14 (dq, J=13.8, 6.9 Hz, 1H), 4.25-4.73 (m, 2H), 5.11 (d, J=7.7 Hz, 1H), 7.33 (br d, J=8.1 Hz, 2H), 7.42 (dd, J=8.1, 2.0 Hz, 1H), 7.68 (d, J=2.0 Hz, 1H), 7.70 (d, J=8.1 Hz, 1H), 7.97 (d, J=8.6 Hz, 2H), 8.28 (br s, 1H); LCMS (method D): Rt 1.62 min

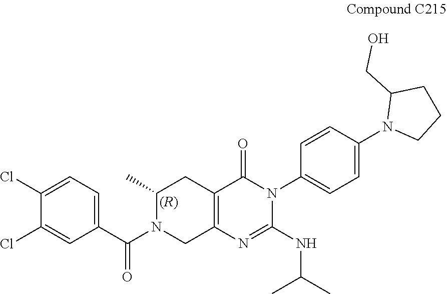

¹H NMR (400 MHz, DMSO-d₆, 100° C.) δ ppm 1.05 (d, J=6.5 Hz, 3H), 1.06 (d, J=6.5 Hz, 3H), 1.18 (d, J=6.6 Hz, 3H), 1.89-2.09 (m, 4H), 2.32 (d, J=16.5 Hz, 1H), 2.49-2.56 (m, 1H), 3.10-3.19 (m, 1H), 3.31 (ddd, J=10.8, 7.5, 6.3 Hz, 1H), 3.37-3.44 (m, 1H), 3.51-3.60 (m, 1H), 3.74-3.82 (m, 1H), 3.99 (br d, J=18.5 Hz, 1H), 4.08 (dq, J=13.5, 6.8 Hz, 1H), 4.37-4.60 (m, 2H), 4.41 (t, J=5.5 Hz, 1H), 4.67 (br d, J=7.9 Hz, 1H), 6.73 (d, J=9.0 Hz, 2H), 6.97 (br d, J=8.4 Hz, 2H), 7.40 (dd, J=8.1, 2.0 Hz, 1H), 7.65 (d, J=2.0 Hz, 1H), 7.68 (d, J=8.1 Hz, 1H); LCMS (method D): Rt 2.10 min

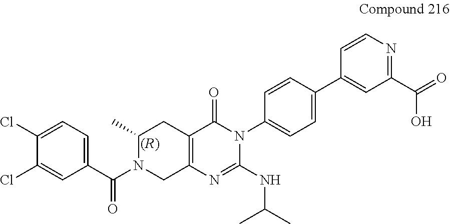

¹H NMR (400 MHz, DMSO-d₆, 100° C.) δ ppm 1.07 (d, J=6.6 Hz, 3H), 1.08 (d, J=6.6 Hz, 3H), 1.20 (d, J=6.8 Hz, 3H), 2.35 (d, J=16.5 Hz, 1H), 2.51-2.60 (m, 1H), 4.02 (br d, J=18.9 Hz, 1H), 4.15 (dq, J=13.5, 6.8 Hz, 1H), 4.37-4.67 (m, 2H), 5.02 (br d, J=7.3 Hz, 1H), 7.39-7.44 (m, 3H), 7.66 (d, J=2.0 Hz, 1H), 7.69 (d, J=8.1 Hz, 1H), 7.87 (br s, 1H), 7.98 (d, J=8.8 Hz, 2H), 8.29 (br s, 1H), 8.71 (br s, 1H); LCMS (method B): Rt 1.68 min

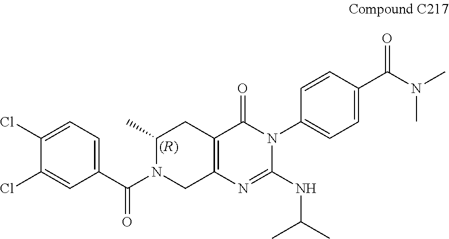

¹H NMR (400 MHz, DMSO-d₆, 81° C.) δ ppm 1.06 (d, J=6.6 Hz, 3H), 1.07 (d, J=6.6 Hz, 3H), 1.18 (d, J=6.8 Hz, 3H), 2.32 (br d, J=16.3 Hz, 1H), 2.53 (br dd, J=16.5, 5.9 Hz, 1H), 2.99 (s, 6H), 4.02 (br d, J=17.8 Hz, 1H), 4.12 (dq, J=13.5, 6.7 Hz, 1H), 4.22-4.89 (m, 2H), 5.05 (d, J=7.7 Hz, 1H), 7.31 (d, J=8.1 Hz, 2H), 7.42 (dd, J=8.1, 2.0 Hz, 1H), 7.55 (d, J=8.6 Hz, 2H), 7.68 (d, J=1.8 Hz, 1H), 7.70 (d, J=8.4 Hz, 1H); LCMS (method B): Rt 1.97 min

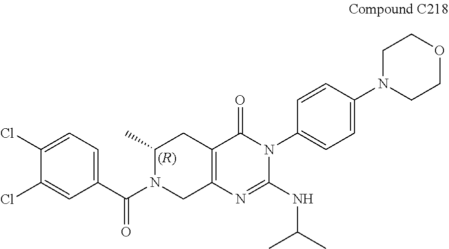

¹H NMR (400 MHz, DMSO-d₆, 100° C.) δ ppm 1.05 (d, J=6.6 Hz, 6H), 1.18 (d, J=6.8 Hz, 3H), 2.32 (d, J=16.5 Hz, 1H), 2.53 (br dd, J=16.5, 5.9 Hz, 1H), 3.18-3.25 (m, 4H), 3.71-3.80 (m, 4H), 4.00 (br d, J=18.7 Hz, 1H), 4.09 (dq, J=13.5, 6.7 Hz, 1H), 4.35-4.65 (m, 2H), 4.70 (br d, J=7.7 Hz, 1H), 7.01-7.09 (m, 4H), 7.40 (dd, J=8.3, 1.9 Hz, 1H), 7.65 (d, J=2.0 Hz, 1H), 7.68 (d, J=8.1 Hz, 1H); LCMS (method B): Rt 2.18 min

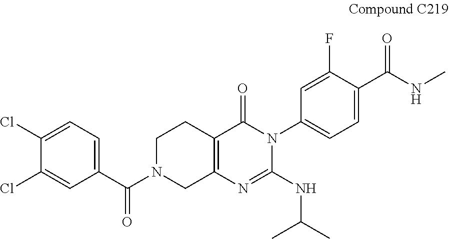 [SMILES: CNC(=O)c1ccc(-n2c(NC(C)C)nc3c(c2=O)CCN(C(=O)c2ccc(Cl)c(Cl)c2)C3)cc1F]

¹H NMR (400 MHz, DMSO-d₆, 100° C.) δ ppm 1.06 (d, J=6.6 Hz, 6H), 2.41 (t, J=5.8 Hz, 2H), 2.83 (s, 3H), 3.63 (br s, 2H), 4.14 (dt, J=13.0, 6.6 Hz, 1H), 4.30 (s, 2H), 5.24 (br s, 1H), 7.14 (dd, J=8.1, 1.8 Hz, 1H), 7.23 (dd, J=10.9, 1.9 Hz, 1H), 7.42 (dd, J=8.1, 2.0 Hz, 1H), 7.67 (d, J=2.0 Hz, 1H), 7.69 (d, J=8.1 Hz, 1H), 7.78 (t, J=7.9 Hz, 1H), 7.98 (br s, 1H); LCMS (method G): Rt 2.00 min

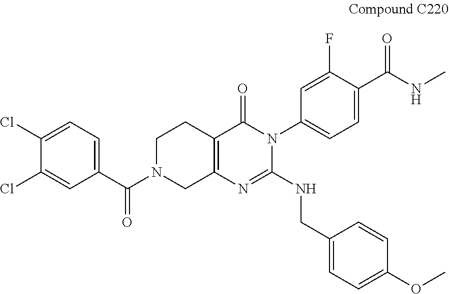 [SMILES: CNC(=O)c1ccc(-n2c(NCc3ccc(OC)cc3)nc3c(c2=O)CCN(C(=O)c2ccc(Cl)c(Cl)c2)C3)cc1F]

¹H NMR (400 MHz, DMSO-d₆, 100° C.) δ ppm 2.41 (t, J=5.7 Hz, 2H), 2.82 (d, J=4.6 Hz, 3H), 3.62 (br s, 2H), 3.72 (s, 3H), 4.28 (s, 2H), 4.34 (s, 2H), 6.38 (br s, 1H), 6.79-6.84 (m, 2H), 7.13-7.19 (m, 3H), 7.26 (dd, J=10.8, 1.8 Hz, 1H), 7.41 (dd, J=8.4, 2.0 Hz, 1H), 7.66 (d, J=2.0 Hz, 1H), 7.68 (d, J=8.4 Hz, 1H), 7.78 (t, J=8.0 Hz, 1H), 7.98 (br s, 1H); LCMS (method C): Rt 1.02 min

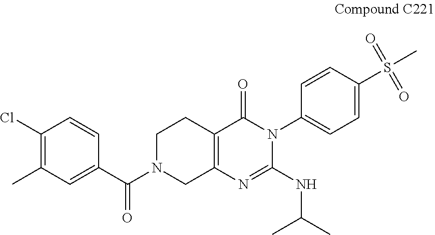 [SMILES: Cc1cc(C(=O)N2CCc3c(nc(NC(C)C)n(-c4ccc(S(C)(=O)=O)cc4)c3=O)C2)ccc1Cl]

¹H NMR (400 MHz, DMSO-d₆, 126° C.) δ ppm 1.10 (d, J=6.4 Hz, 6H), 2.39 (s, 3H), 2.41 (t, J=5.9 Hz, 2H), 3.23 (s, 3H), 3.64 (t, J=5.8 Hz, 2H), 4.13 (dq, J=13.6, 6.6 Hz, 1H), 4.31 (s, 2H), 5.06 (br d, J=7.3 Hz, 1H), 7.26 (dd, J=8.1, 1.5 Hz, 1H), 7.39 (d, J=1.8 Hz, 1H), 7.44 (d, J=8.1 Hz, 1H), 7.49-7.55 (m, 2H), 8.04-8.09 (m, 2H); LCMS (method B): Rt 1.96 min

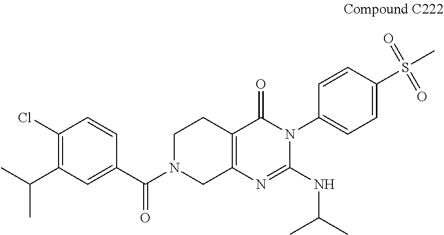 [SMILES: CC(C)Nc1nc2c(c(=O)n1-c1ccc(S(C)(=O)=O)cc1)CCN(C(=O)c1ccc(Cl)c(C(C)C)c1)C2]

¹H NMR (400 MHz, DMSO-d₆, 126° C.) δ ppm 1.07 (d, J=6.5 Hz, 6H), 1.25 (d, J=7.0 Hz, 6H), 2.42 (t, J=5.8 Hz, 2H), 3.23 (s, 3H), 3.38 (dt, J=13.7, 6.8 Hz, 1H), 3.63 (t, J=5.8 Hz, 2H), 4.13 (dq, J=13.7, 6.7 Hz, 1H), 4.32 (s, 2H), 5.08 (br d, J=7.5 Hz, 1H), 7.26 (dd, J=8.1, 2.0 Hz, 1H), 7.40 (d, J=2.2 Hz, 1H), 7.45 (d, J=8.1 Hz, 1H), 7.49-7.54 (m, 2H), 8.03-8.09 (m, 2H); LCMS (method D): Rt 2.07 min

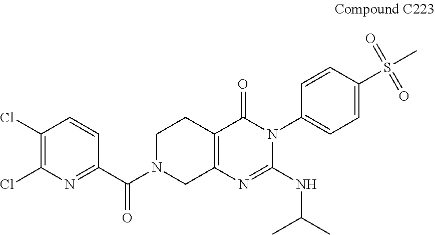 [SMILES: CC(C)Nc1nc2c(c(=O)n1-c1ccc(S(C)(=O)=O)cc1)CCN(C(=O)c1ccc(Cl)c(Cl)n1)C2]

¹H NMR (400 MHz, DMSO-d₆, 150° C.) δ ppm 1.09 (d, J=6.6 Hz, 6H), 2.45-2.54 (m, 2H), 3.21 (s, 3H), 3.72 (t, J=5.8 Hz, 2H), 4.13 (dq, J=13.2, 6.4 Hz, 1H), 4.40 (s, 2H), 4.88 (br d, J=6.4 Hz, 1H), 7.49-7.54 (m, 2H), 7.64 (d, J=8.1 Hz, 1H), 8.03-8.09 (m, 2H), 8.14 (d, J=8.1 Hz, 1H); LCMS (method B): Rt 1.87 min

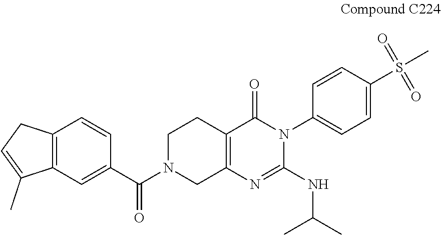 [SMILES: CC1=CCc2ccc(C(=O)N3CCc4c(nc(NC(C)C)n(-c5ccc(S(C)(=O)=O)cc5)c4=O)C3)cc21]

¹H NMR (400 MHz, DMSO-d₆, 101° C.) δ ppm 1.08 (d, J=6.6 Hz, 6H), 2.26 (d, J=1.3 Hz, 3H), 2.42 (br t, J=5.8 Hz, 2H), 3.25 (s, 3H), 3.69 (t, J=5.8 Hz, 2H), 4.13 (dq, J=14.0, 6.8 Hz, 1H), 4.36 (s, 2H), 5.21 (br d, J=7.7 Hz, 1H), 7.38 (dd, J=8.4, 1.8 Hz, 1H), 7.50-7.55 (m, 2H), 7.57 (dd, J=8.4, 0.4 Hz, 1H), 7.68 (d, J=1.3 Hz, 1H), 7.76 (d, J=1.3 Hz, 1H), 8.05-8.09 (m, 2H); LCMS (method B): Rt 1.86 min

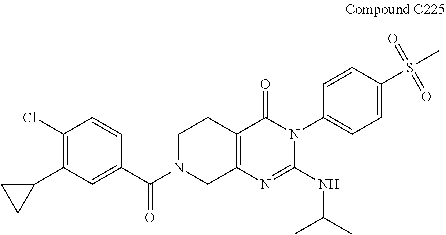 [SMILES: CC(C)Nc1nc2c(c(=O)n1-c1ccc(S(C)(=O)=O)cc1)CCN(C(=O)c1ccc(Cl)c(C3CC3)c1)C2]

¹H NMR (400 MHz, DMSO-d₆, 126° C.) δ ppm 0.69-0.75 (m, 2H), 1.00-1.06 (m, 2H), 1.07 (d, J=6.6 Hz, 6H), 2.14-2.27 (m, 1H), 2.40 (br t, J=6.1 Hz, 2H), 3.19 (s, 3H), 3.64 (br t, J=5.8 Hz, 2H), 4.07-4.19 (m, 1H), 4.29 (s, 2H), 5.12 (br d, J=1.5 Hz, 1H), 7.06 (d, J=1.5 Hz, 1H), 7.23 (dd, J=8.3, 1.9 Hz, 1H), 7.46 (d, J=8.1 Hz, 1H), 7.52 (d, J=8.6 Hz, 2H), 8.07 (d, J=8.4 Hz, 2H); LCMS (method D): Rt 2.04 min

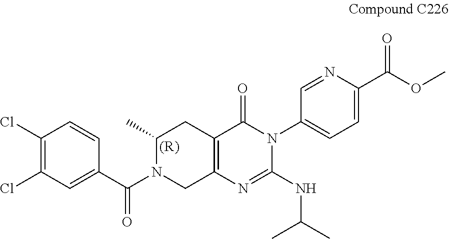

¹H NMR (400 MHz, DMSO-d₆, 81° C.) δ ppm 1.06 (d, J=6.5 Hz, 3H), 1.06 (d, J=6.5 Hz, 3H), 1.19 (d, J=6.6 Hz, 3H), 2.32 (br d, J=16.3 Hz, 1H), 2.54 (br dd, J=16.4, 6.1 Hz, 1H), 3.93 (s, 3H), 4.03 (br d, J=18.3 Hz, 1H), 4.17 (dq, J=13.8, 6.7 Hz, 1H), 4.28-4.91 (m, 2H), 5.73 (br d, J=7.7 Hz, 1H), 7.41 (dd, J=8.1, 2.0 Hz, 1H), 7.68 (d, J=1.8 Hz, 1H), 7.70 (d, J=8.4 Hz, 1H), 7.92 (dd, J=8.4, 2.4 Hz, 1H), 8.18 (dd, J=8.4, 0.7 Hz, 1H), 8.57 (d, J=2.0 Hz, 1H); LCMS (method D): Rt 2.01 min

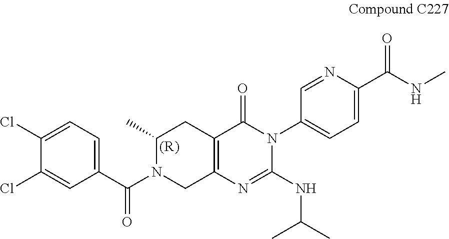

¹H NMR (400 MHz, DMSO-d₆, 81° C.) δ ppm 1.05 (d, J=6.6 Hz, 3H), 1.06 (d, J=6.6 Hz, 3H), 1.19 (d, J=6.8 Hz, 3H), 1.23 (d, J=6.6 Hz, 6H), 2.33 (br d, J=16.3 Hz, 1H), 2.54 (br dd, J=16.5, 6.2 Hz, 1H), 4.03 (br d, J=18.9 Hz, 1H), 4.08-4.25 (m, 2H), 4.28-4.83 (m, 2H), 5.72 (br d, J=7.9 Hz, 1H), 7.41 (dd, J=8.1, 2.0 Hz, 1H), 7.67 (d, J=2.0 Hz, 1H), 7.70 (d, J=8.1 Hz, 1H), 7.89 (dd, J=8.4, 2.2 Hz, 1H), 8.17 (d, J=8.4 Hz, 1H), 8.33 (br d, J=8.1 Hz, 1H), 8.48 (d, J=2.0 Hz, 1H); LCMS (method D): Rt 2.18 min

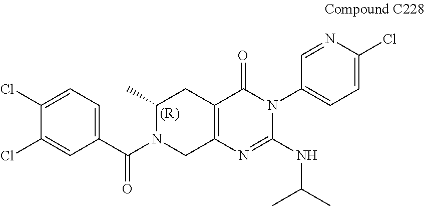

¹H NMR (400 MHz, DMSO-d₆, 81° C.) δ ppm 1.06 (br d, J=6.6 Hz, 1H), 1.07 (br d, J=6.6 Hz, 3H), 1.15-1.21 (m, 5H), 2.32 (d, J=16.3 Hz, 1H), 2.53 (br dd, J=16.5, 6.2 Hz, 1H), 4.02 (br d, J=18.9 Hz, 1H), 4.17 (dq, J=13.4, 6.6 Hz, 1H), 4.30-4.76 (m, 2H), 5.82 (d, J=7.9 Hz, 1H), 7.41 (dd, J=8.1, 2.0 Hz, 1H), 7.65 (dd, J=8.4, 0.4 Hz, 1H), 7.67 (d, J=1.8 Hz, 1H), 7.70 (d, J=8.1 Hz, 1H), 7.79 (dd, J=8.5, 2.3 Hz, 1H), 8.30 (d, J=2.6 Hz, 1H); LCMS (method C): Rt 1.13 min

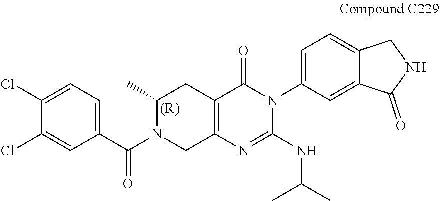

¹H NMR (400 MHz, DMSO-d₆, 100° C.) δ ppm 1.05 (d, J=6.6 Hz, 3H), 1.05 (d, J=6.6 Hz, 3H), 1.19 (d, J=6.8 Hz, 3H), 2.33 (d, J=16.5 Hz, 1H), 2.55 (br dd, J=16.3, 5.9 Hz, 1H), 4.02 (br d, J=18.7 Hz, 1H), 4.14 (dq, J=13.7, 6.7 Hz, 1H), 4.44 (s, 2H), 4.45-4.58 (m, 2H), 5.06 (br d, J=7.7 Hz, 1H), 7.41 (dd, J=8.3, 1.9 Hz, 2H), 7.49 (s, 1H), 7.66 (d, J=1.8 Hz, 1H), 7.67-7.73 (m, 2H), 8.32 (br s, 1H); LCMS (method D): Rt 1.89 min

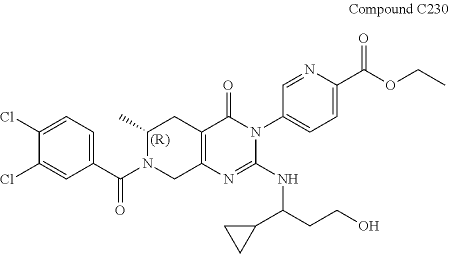

¹H NMR (400 MHz, DMSO-d₆, 81° C.) δ ppm 0.11-0.19 (m, 1H), 0.26-0.42 (m, 3H), 0.81-0.97 (m, 1H), 1.13-1.22 (m, 3H), 1.35 (t, J=7.1 Hz, 3H), 1.58-1.74 (m, 2H), 2.32 (br d, J=16.7 Hz, 1H), 2.50-2.59 (m, 1H), 3.53-3.64 (m, 1H), 3.37-3.51 (m, 2H), 3.86-4.06 (m, 1H), 4.06-4.22 (m, 1H), 4.27-4.67 (m, 2H), 4.37 (q, J=7.1 Hz, 2H), 5.46-5.63 (m, 1H), 7.36-7.45 (m, 3H), 7.66-7.68 (m, 1H), 7.70 (dd, J=8.3, 0.6 Hz, 1H), 8.10 (d, J=8.6 Hz, 2H); LCMS (method C): Rt 1.11 min

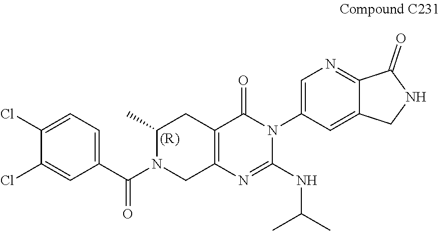

¹H NMR (400 MHz, DMSO-d₆, 100° C.) δ ppm 1.05 (d, J=6.4 Hz, 3H), 1.05 (d, J=6.4 Hz, 3H), 1.19 (d, J=6.8 Hz, 3H), 2.34 (d, J=16.4 Hz, 1H), 2.54 (br dd, J=16.3, 5.9 Hz, 1H), 4.02 (br d, J=18.5 Hz, 1H), 4.15 (dq, J=13.6, 6.8 Hz, 1H), 4.41 (s, 2H), 4.43-4.60 (m, 2H), 5.06 (br d, J=7.9 Hz, 1H), 7.31 (br d, J=7.9 Hz, 1H), 7.41 (dd, J=8.1, 2.0 Hz, 1H), 7.45 (br s, 1H), 7.66 (d, J=2.0 Hz, 1H), 7.69 (d, J=8.1 Hz, 1H), 7.81 (d, J=7.9 Hz, 1H), 8.31 (s, 1H); LCMS (method B): Rt 1.85 min

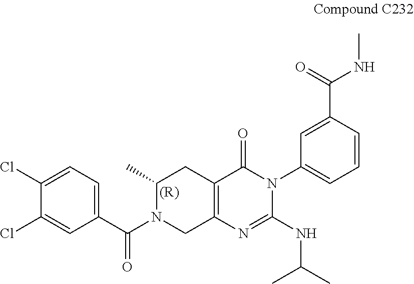

¹H NMR (400 MHz, DMSO-d₆, 100° C.) δ ppm 1.04-1.08 (m, 6H), 1.19 (d, J=6.6 Hz, 3H), 2.34 (d, J=16.5 Hz, 1H), 2.55 (br dd, J=16.5, 5.9 Hz, 1H), 2.81 (d, J=4.6 Hz, 3H), 4.02 (br d, J=18.7 Hz, 1H), 4.12 (dq, J=13.7, 6.4 Hz, 1H), 4.38-4.64 (s, 2H), 4.95 (br d, J=7.7 Hz, 1H), 7.38 (br d, J=8.4 Hz, 1H), 7.41 (dd, J=8.3, 1.4 Hz, 1H), 7.62 (t, J=7.9 Hz, 1H), 7.66 (d, J=2.0 Hz, 1H), 7.69 (d, J=8.1 Hz, 1H), 7.71 (br s, 1H), 7.97 (dt, J=8.0, 1.3 Hz, 1H), 8.18-8.24 (m, 1H); LCMS (method C): Rt 0.82 min

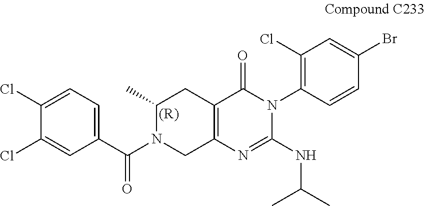

¹H NMR (400 MHz, DMSO-d₆, 80° C.) δ ppm 1.04-1.09 (m, 6H), 1.17 (d, J=6.8 Hz, 3H), 2.27-2.37 (m, 1H), 2.50-2.58 (m, 1H), 3.95-4.10 (m, 1H), 4.20 (dq, J=13.2, 6.6 Hz, 1H), 4.28-4.72 (m, 2H), 5.68 (d, J=7.9 Hz, 1H), 7.31-7.45 (m, 2H), 7.66-7.72 (m, 3H), 7.89-7.92 (m, 1H); LCMS (method C): Rt 1.28 min

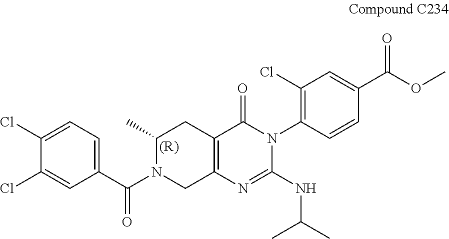

¹H NMR (400 MHz, DMSO-d₆) δ ppm 1.0-1.10 (m, 6H), 1.14-1.19 (m, 3H), 1.22-1.31 (m, 1H), 2.28-2.40 (m, 1H), 2.51-2.61 (m, 1H), 3.89-3.94 (m, 3H), 3.95-4.11 (m, 1H), 4.15-4.27 (m, 1H), 5.56-5.63 (m, 1H), 7.39-7.46 (m, 1H), 7.53-7.61 (m, 1H), 7.66-7.73 (m, 2H), 8.01-8.06 (m, 1H), 8.11-8.14 (m, 1H); LCMS (method C): Rt 1.23 min

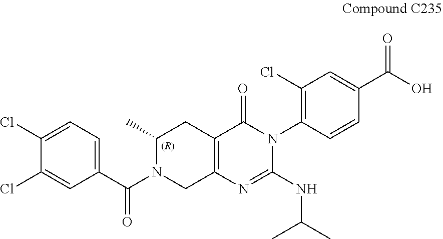

¹H NMR (400 MHz, DMSO-d₆, 80° C.) δ ppm 1.02-1.10 (m, 6H), 1.18 (br d, J=6.6 Hz, 3H), 2.33 (br d, J=16.5 Hz, 1H), 2.54 (br dd, J=16.2, 6.7 Hz, 1H), 3.96-4.11 (m, 1H), 4.21 (dq, J=13.4, 6.6 Hz, 1H), 4.31-4.69 (m, 2H), 5.59 (br d, J=7.9 Hz, 1H), 7.39-7.46 (m, 1H), 7.49-7.58 (m, 1H), 7.65-7.74 (m, 2H), 8.02 (dd, J=8.3, 1.7 Hz, 1H), 8.10 (s, 1H); LCMS (method C): Rt 0.84 min

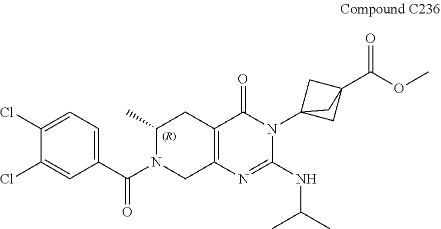

¹H NMR (400 MHz, DMSO-d₆, 81° C.) δ ppm 1.11 (d, J=6.8 Hz, 3H), 1.15-1.23 (m, 6H), 2.23-2.31 (m, 1H), 2.41-2.50 (m, 1H), 2.74 (s, 6H), 3.65 (s, 3H), 3.90 (br d, J=18.7 Hz, 1H), 4.09 (dq, J=13.3, 6.7 Hz, 1H), 4.18-4.80 (m, 2H), 5.44 (br d, J=7.4 Hz, 1H), 7.38 (dd, J=8.2, 1.9 Hz, 1H), 7.64 (d, J=2.0 Hz, 1H), 7.68 (d, J=8.1 Hz, 1H); LCMS (method C): Rt 1.19 min

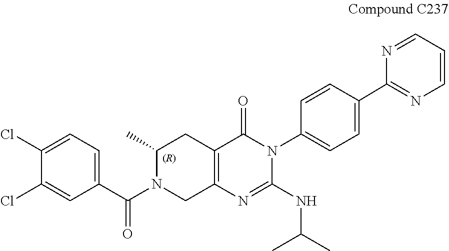

¹H NMR (400 MHz, DMSO-d₆, 100° C.) δ ppm 1.06 (d, J=6.5 Hz, 3H), 1.06 (d, J=6.5 Hz, 3H), 1.20 (d, J=6.9 Hz, 3H), 2.32-2.38 (m, 1H), 2.54-2.60 (m, 1H), 4.03 (br d, J=18.8 Hz, 1H), 4.11-4.18 (m, 1H), 4.40-4.60 (m, 2H), 5.02 (br d, J=8.6 Hz, 1H), 7.37-7.46 (m, 4H), 7.67 (d, J=2.0 Hz, 1H), 7.69 (d, J=8.2 Hz, 1H), 8.51-8.56 (m, 2H), 8.92 (d, J=4.9 Hz, 2H); LCMS (method C): Rt 1.16 min

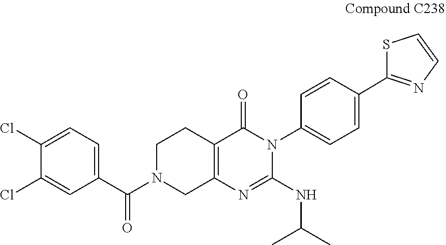 [SMILES: CC(C)Nc1nc2c(c(=O)n1-c1ccc(-c3nccs3)cc1)CCN(C(=O)c1ccc(Cl)c(Cl)c1)C2]

¹H NMR (400 MHz, DMSO-d₆, 100° C.) δ ppm 1.06 (d, J=6.6 Hz, 6H), 2.42 (t, J=5.7 Hz, 2H), 3.58-3.68 (m, 2H), 4.10-4.19 (m, 1H), 4.31 (s, 2H), 5.11 (br d, J=7.5 Hz, 1H), 7.34-7.38 (m, 2H), 7.43 (dd, J=8.1, 2.0 Hz, 1H), 7.67-7.71 (m, 2H), 7.77 (d, J=3.3 Hz, 1H), 7.95 (d, J=3.3 Hz, 1H), 8.08-8.11 (m, 2H); LCMS (method C): Rt 1.16 min

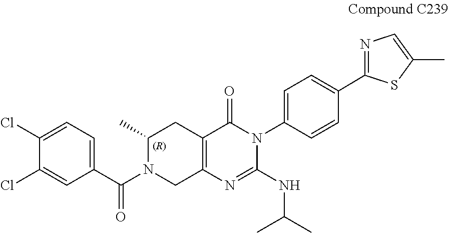

¹H NMR (400 MHz, DMSO-d₆, 100° C.) δ ppm 1.06 (d, J=6.6 Hz, 3H), 1.07 (d, J=6.6 Hz, 3H), 1.19 (d, J=6.8 Hz, 3H), 2.34 (d, J=16.3 Hz, 1H), 2.52 (d, J=1.1 Hz, 3H), 2.53-2.59 (m, 1H), 4.02 (br d, J=18.7 Hz, 1H), 4.14 (dq, J=13.7, 6.6 Hz, 1H), 4.39-4.67 (m, 2H), 5.09 (br d, J=7.9 Hz, 1H), 7.31-7.37 (m, 2H), 7.41 (dd, J=8.1, 2.0 Hz, 1H), 7.62 (d, J=1.1 Hz, 1H), 7.66 (d, J=2.0 Hz, 1H), 7.69 (d, J=8.1 Hz, 1H), 8.01 (d, J=8.6 Hz, 2H); LCMS (method C): Rt 1.25 min

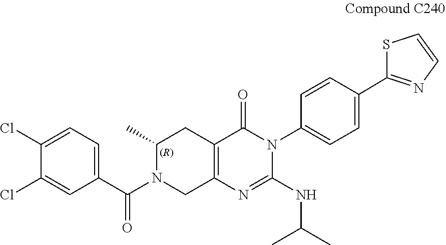

¹H NMR (400 MHz, DMSO-d₆, 100° C.) δ ppm 1.06 (d, J=6.6 Hz, 3H), 1.07 (d, J=6.6 Hz, 3H), 1.19 (d, J=6.8 Hz, 3H), 2.34 (d, J=16.5 Hz, 1H), 2.55 (br dd, J=16.4, 6.1 Hz, 1H), 4.02 (br d, J=18.3 Hz, 1H), 4.15 (dq, J=13.6, 6.8 Hz, 1H), 4.39-4.66 (m, 2H), 5.10 (br d, J=7.9 Hz, 1H), 7.37 (d, J=8.4 Hz, 2H), 7.41 (dd, J=8.1, 2.0 Hz, 1H), 7.66 (d, J=1.8 Hz, 1H), 7.69 (d, J=8.1 Hz, 1H), 7.77 (d, J=3.1 Hz, 1H), 7.95 (d, J=3.3 Hz, 1H), 8.10 (d, J=8.8 Hz, 2H); LCMS (method C): Rt 1.19 min

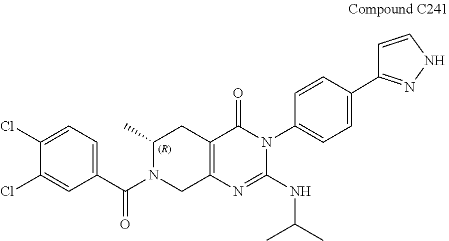

¹H NMR (400 MHz, CDCl₃, 47° C.) δ ppm 1.09 (d, J=6.4 Hz, 6H), 1.26 (d, J=6.6 Hz, 3H), 2.55 (d, J=16.5 Hz, 1H), 2.70 (br dd, J=16.3, 5.5 Hz, 1H), 3.93 (br d, J=7.7 Hz, 1H), 4.04 (br d, J=19.2 Hz, 1H), 4.16 (dq, J=13.1, 6.5 Hz, 1H), 4.30-5.17 (m, 2H), 6.64 (d, J=2.2 Hz, 1H), 7.22-7.30 (m, 3H), 7.51 (d, J=8.1 Hz, 1H), 7.54 (d, J=2.0 Hz, 1H), 7.60 (d, J=2.4 Hz, 1H), 7.92-7.97 (m, 2H); LCMS (method C): Rt 1.08 min

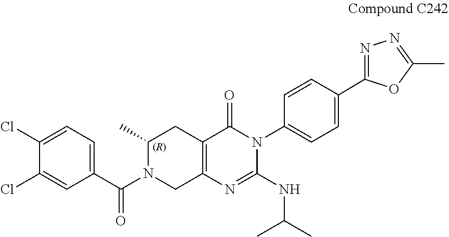

¹H NMR (400 MHz, DMSO-d₆, 120° C.) δ ppm 1.06 (d, J=6.5 Hz, 3H), 1.07 (d, J=6.9 Hz, 3H), 1.19 (d, J=6.9 Hz, 3H), 2.35 (d, J=16.3 Hz, 1H), 2.51-2.55 (m, 1H), 2.59 (s, 3H), 4.02 (br d, J=18.8 Hz, 1H), 4.14 (dt, J=13.1, 6.5 Hz, 1H), 4.46-4.59 (m, 2H), 4.99 (br s, 1H), 7.40 (dd, J=8.2, 2.0 Hz, 1H), 7.44-7.49 (m, 2H), 7.64 (d, J=2.0 Hz, 1H), 7.68 (d, J=8.1 Hz, 1H), 8.09-8.12 (m, 2H); LCMS (method C): Rt 1.08 min

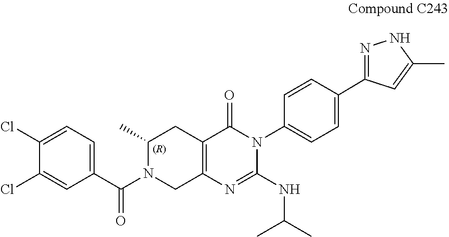

¹H NMR (400 MHz, DMSO-d₆, 100° C.) δ ppm 1.05 (d, J=6.6 Hz, 3H), 1.06 (d, J=6.6 Hz, 3H), 1.19 (d, J=6.8 Hz, 3H), 2.28 (s, 3H), 2.34 (d, J=16.3 Hz, 1H), 2.50-2.58 (m, 1H), 4.01 (br d, J=18.3 Hz, 1H), 4.12 (dt, J=12.9, 6.3 Hz, 1H), 4.36-4.65 (m, 2H), 4.89 (br s, 1H), 6.45 (s, 1H), 7.24 (d, J=8.4 Hz, 2H), 7.41 (dd, J=8.3, 1.9 Hz, 1H), 7.66 (d, J=1.8 Hz, 1H), 7.69 (d, J=8.1 Hz, 1H), 7.89 (d, J=8.8 Hz, 2H); LCMS (method C): Rt 1.12 min

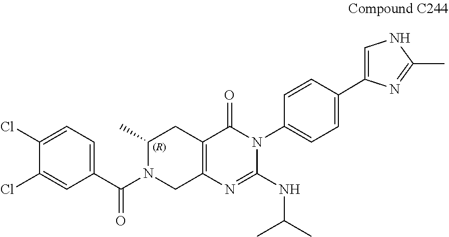

¹H NMR (400 MHz, DMSO-d₆, 120° C.) δ ppm 1.03-1.07 (m, 6H), 1.19 (d, J=6.5 Hz, 3H), 2.31-2.37 (m, 4H), 2.54-2.59 (m, 1H), 3.97-4.10 (m, 2H), 4.47-4.57 (m, 2H), 4.62-4.74 (m, 1H), 7.14-7.17 (m, 2H), 7.38-7.43 (m, 2H), 7.64 (d, J=2.0 Hz, 1H), 7.67 (d, J=8.2 Hz, 1H), 7.85-7.89 (m, 2H); LCMS (method C): Rt 1.02 min

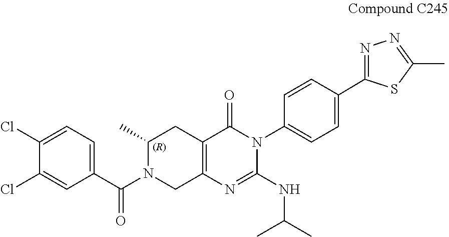

¹H NMR (400 MHz, DMSO-d₆, 120° C.) δ ppm 1.07 (d, J=6.5 Hz, 3H), 1.08 (d, J=6.6 Hz, 3H), 1.20 (d, J=6.9 Hz, 3H), 2.35 (d, J=16.7 Hz, 1H), 2.51-2.59 (m, 1H), 2.79 (s, 3H), 4.02 (br d, J=19.2 Hz, 1H), 4.09-4.21 (m, 1H), 4.45-4.58 (m, 2H), 5.04 (br d, J=8.6 Hz, 1H), 7.38-7.43 (m, 3H), 7.64 (d, J=2.0 Hz, 1H), 7.68 (d, J=8.2 Hz, 1H), 8.04-8.08 (m, 2H); LCMS (method C): Rt 1.12 min

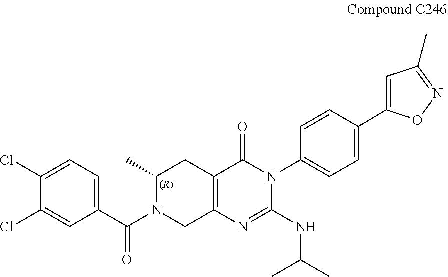

¹H NMR (400 MHz, DMSO-d₆, 100° C.) δ ppm 1.06 (d, J=6.5 Hz, 3H), 1.07 (d, J=6.6 Hz, 3H), 1.19 (d, J=6.8 Hz, 3H), 2.31 (s, 3H), 2.34 (br d, J=16.3 Hz, 1H), 2.54 (br dd, J=16.5, 6.2 Hz, 1H), 4.02 (br d, J=18.9 Hz, 1H), 4.09-4.23 (m, 1H), 4.40-4.63 (m, 2H), 5.08 (br s, 1H), 6.86 (s, 1H), 7.37-7.43 (m, 3H), 7.66 (d, J=2.0 Hz, 1H), 7.69 (d, J=8.1 Hz, 1H), 7.95 (d, J=8.6 Hz, 2H); LCMS (method C): Rt 1.19 min

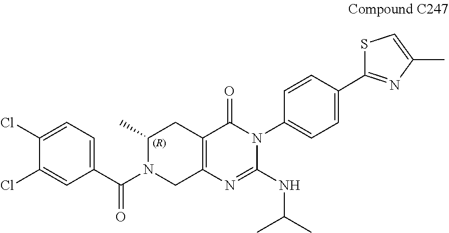

¹H NMR (400 MHz, DMSO-d₆, 100° C.) δ ppm 1.06 (d, J=6.5 Hz, 3H), 1.07 (d, J=6.5 Hz, 3H), 1.19 (d, J=6.8 Hz, 3H), 2.34 (br d, J=16.3 Hz, 1H), 2.45 (d, J=0.9 Hz, 3H), 2.51-2.59 (m, 1H), 4.02 (br d, J=18.5 Hz, 1H), 4.14 (dq, J=13.2, 6.2 Hz, 1H), 4.38-4.69 (m, 2H), 5.06 (br d, J=7.9 Hz, 1H), 7.30 (d, J=0.9 Hz, 1H), 7.33-7.37 (m, 2H), 7.41 (dd, J=8.1, 2.0 Hz, 1H), 7.66 (d, J=2.0 Hz, 1H), 7.69 (d, J=8.1 Hz, 1H), 8.05 (br d, J=8.8 Hz, 2H); LCMS (method C): Rt 1.25 min

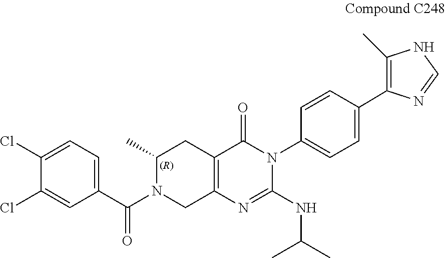

¹H NMR (400 MHz, DMSO-d₆, 100° C.) δ ppm 1.01-1.11 (m, 6H), 1.19 (br d, J=6.6 Hz, 3H), 2.35 (br d, J=16.5 Hz, 1H), 2.39-2.47 (m, 3H), 2.56 (br dd, J=16.5, 5.5 Hz, 1H), 4.02 (br d, J=18.5 Hz, 1H), 4.12 (dq, J=13.3, 6.6 Hz, 1H), 4.42-4.62 (m, 2H), 4.83 (br s, 1H), 7.23 (br s, 2H), 7.41 (br d, J=8.1 Hz, 1H), 7.53 (s, 1H), 7.63-7.76 (m, 2H), 7.83 (br s, 2H), 11.77 (br s, 1H); LCMS (method C): Rt 1.04 min

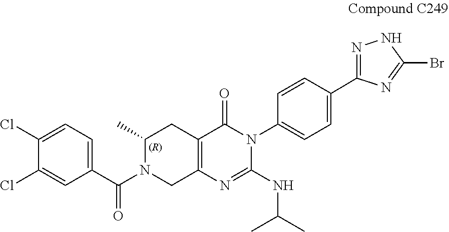

¹H NMR (400 MHz, DMSO-d₆, 100° C.) δ ppm 1.05 (d, J=6.6 Hz, 3H), 1.06 (d, J=6.6 Hz, 3H), 1.19 (d, J=6.8 Hz, 3H), 2.34 (br d, J=16.5 Hz, 1H), 2.55 (br dd, J=16.5, 6.2 Hz, 1H), 4.02 (br d, J=18.7 Hz, 1H), 4.14 (ddd, J=14.1, 7.5, 6.6 Hz, 1H), 4.37-4.65 (m, 2H), 5.06 (br d, J=7.7 Hz, 1H), 7.37-7.44 (m, 3H), 7.66 (d, J=2.0 Hz, 1H), 7.69 (d, J=8.4 Hz, 1H), 8.10 (d, J=8.8 Hz, 2H); LCMS (method C): Rt 1.04 min

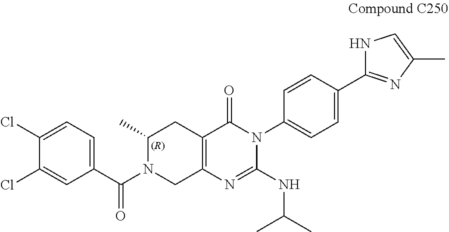

¹H NMR (400 MHz, DMSO-d₆, 100° C.) δ ppm 1.05 (d, J=6.6 Hz, 3H), 1.06 (d, J=6.6 Hz, 3H), 1.19 (d, J=6.8 Hz, 3H), 2.22 (s, 3H), 2.34 (d, J=16.3 Hz, 1H), 2.55 (br dd, J=16.4, 6.1 Hz, 1H), 4.02 (br d, J=18.7 Hz, 1H), 4.12 (dq, J=13.7, 6.6 Hz, 1H), 4.39-4.65 (m, 2H), 4.88 (br d, J=7.7 Hz, 1H), 6.82 (br s, 1H), 7.27 (d, J=8.4 Hz, 2H), 7.41 (dd, J=8.4, 2.0 Hz, 1H), 7.66 (d, J=2.0 Hz, 1H), 7.69 (d, J=8.1 Hz, 1H), 8.03 (d, J=8.8 Hz, 2H), 12.05 (br s, 1H); LCMS (method C): Rt 1.04 min

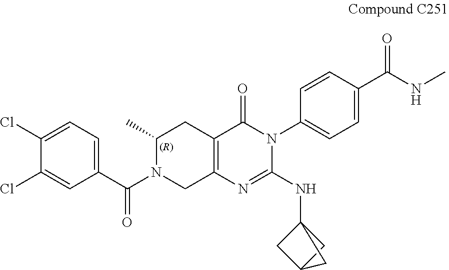

¹H NMR (400 MHz, DMSO-d₆, 81° C.) δ ppm 1.17 (d, J=6.8 Hz, 3H), 2.02 (s, 6H), 2.31 (d, J=16.5 Hz, 1H), 2.37 (s, 1H), 2.53 (br dd, J=16.5, 5.9 Hz, 1H), 2.83 (d, J=4.6 Hz, 3H), 3.99 (br d, J=18.5 Hz, 1H), 4.20-4.90 (m, 2H), 6.26 (s, 1H), 7.31 (br d, J=8.1 Hz, 2H), 7.42 (dd, J=8.1, 2.0 Hz, 1H), 7.68 (d, J=2.0 Hz, 1H), 7.70 (d, J=8.4 Hz, 1H), 7.95 (d, J=8.8 Hz, 2H), 8.26-8.36 (m, 1H); LCMS (method C): Rt 1.09 min

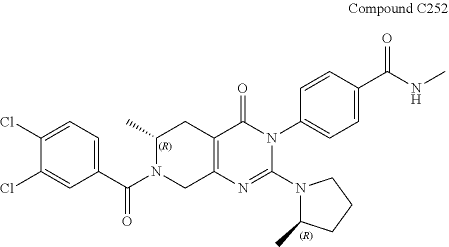

¹H NMR (400 MHz, DMSO-d₆, 81° C.) δ ppm 1.15 (d, J=6.3 Hz, 3H), 1.19 (d, J=6.8 Hz, 3H), 1.21-1.33 (m, 1H), 1.45-1.67 (m, 2H), 1.88-1.98 (m, 1H), 2.24 (td, J=9.9, 6.7 Hz, 1H), 2.34 (br d, J=16.5 Hz, 1H), 2.49-2.60 (m, 1H), 2.81 (d, J=4.6 Hz, 3H), 2.92-3.12 (m, 1H), 3.91-4.13 (m, 2H), 4.19-4.87 (m, 2H), 7.20-7.58 (m, 2H), 7.42 (dd, J=8.1, 2.0 Hz, 1H), 7.68 (d, J=2.0 Hz, 1H), 7.70 (d, J=8.1 Hz, 1H), 7.90 (d, J=8.4 Hz, 2H), 8.25-8.33 (m, 1H); LCMS (method C): Rt 1.06 min

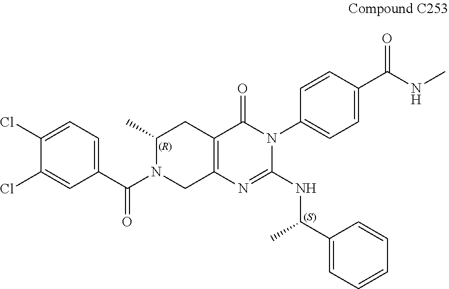

¹H NMR (400 MHz, DMSO-d₆, 81° C.) δ ppm 1.18 (d, J=6.6 Hz, 3H), 1.36 (d, J=7.0 Hz, 3H), 2.30 (d, J=16.5 Hz, 1H), 2.52 (br d, J=5.9 Hz, 1H), 2.83 (d, J=4.4 Hz, 3H), 3.98 (br d, J=19.4 Hz, 1H), 4.13-4.81 (m, 2H), 5.21 (quin, J=7.2 Hz, 1H), 5.70 (d, J=7.7 Hz, 1H), 7.14-7.21 (m, 1H), 7.22-7.27 (m, 4H), 7.31-7.45 (m, 2H), 7.38 (dd, J=8.4, 2.0 Hz, 1H), 7.64 (d, J=2.0 Hz, 1H), 7.68 (d, J=8.4 Hz, 1H), 8.00 (d, J=8.8 Hz, 2H), 8.28-8.39 (m, 1H); LCMS (method C): Rt 1.11 min

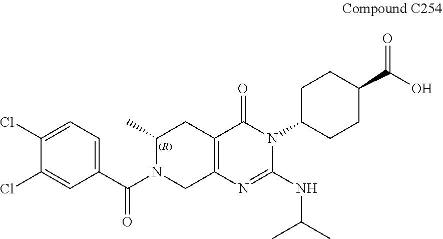

¹H NMR (400 MHz, DMSO-d₆, 100° C.) δ ppm 1.11 (d, J=6.8 Hz, 3H), 1.19 (d, J=6.6 Hz, 3H), 1.20 (d, J=6.6 Hz, 3H), 1.48-1.62 (m, 4H), 1.94 (br d, J=12.1 Hz, 2H), 2.01-2.12 (m, 1H), 2.29 (br d, J=16.3 Hz, 1H), 2.44-2.58 (m, 2H), 2.88-3.70 (m, 2H), 3.88 (br d, J=18.7 Hz, 1H), 4.18 (dq, J=13.3, 6.7 Hz, 1H), 4.23-4.35 (m, 1H), 4.35-4.55 (m, 2H), 6.18 (br d, J=7.0 Hz, 1H), 7.37 (dd, J=8.2, 2.0 Hz, 1H), 7.62 (d, J=1.8 Hz, 1H), 7.67 (d, J=8.1 Hz, 1H); LCMS (method D): Rt 1.62 min

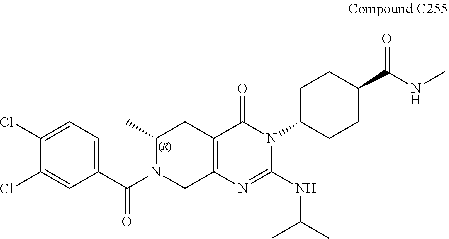

¹H NMR (400 MHz, DMSO-d₆, 100° C.) δ ppm 1.11 (d, J=6.8 Hz, 3H), 1.18 (d, J=6.6 Hz, 3H), 1.19 (d, J=6.6, 3H), 1.55-1.68 (m, 4H), 1.74-1.82 (m, 2H), 2.09-2.18 (m, 1H), 2.28 (br d, J=16.3 Hz, 1H), 2.43 (br d, J=6.2 Hz, 1H), 2.50-2.57 (m, 2H), 2.58 (d, J=4.6 Hz, 3H), 3.88 (br d, J=18.7 Hz, 1H), 4.13-4.23 (m, 2H), 4.30-4.54 (m, 2H), 6.23 (br d, J=7.0 Hz, 1H), 7.26-7.35 (m, 1H), 7.37 (dd, J=8.3, 1.9 Hz, 1H), 7.62 (d, J=1.8 Hz, 1H), 7.67 (d, J=8.1 Hz, 1H); LCMS (method D): Rt 1.94 min

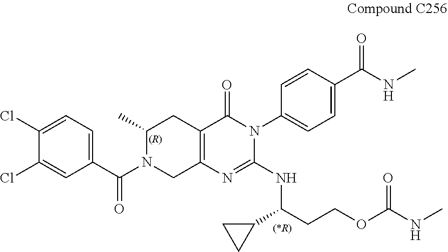

¹H NMR (400 MHz, DMSO-d₆, 81° C.) δ ppm 0.13-0.21 (m, 1H), 0.22-0.34 (m, 2H), 0.35-0.49 (m, 1H), 0.86-0.99 (m, 1H), 1.17 (d, J=6.8 Hz, 3H), 1.82 (q, J=6.8 Hz, 2H), 2.32 (br d, J=16.3 Hz, 1H), 2.44-2.59 (m, 4H), 2.83 (d, J=4.5 Hz, 3H), 3.47-3.59 (m, 1H), 3.81-4.05 (m, 3H), 4.19-4.83 (m, 2H), 5.28 (br d, J=8.4 Hz, 1H), 6.45 (br s, 1H), 7.35 (br d, J=8.1 Hz, 2H), 7.41 (dd, J=8.3, 2.0 Hz, 1H), 7.67 (d, J=2.0 Hz, 1H), 7.69 (d, J=8.1 Hz, 1H), 7.99 (d, J=8.4 Hz, 2H), 8.28-8.38 (m, 1H); LCMS (method D): Rt 1.88 min

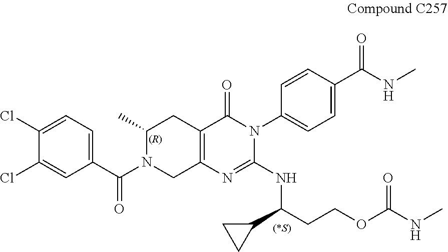

¹H NMR (400 MHz, DMSO-d₆, 81° C.) δ ppm 0.14-0.34 (m, 3H), 0.36-0.47 (m, 1H), 0.77-0.99 (m, 1H), 1.17 (d, J=6.8 Hz, 3H), 1.76-1.89 (m, 2H), 2.31 (br d, J=16.5 Hz, 1H), 2.50-2.59 (m, 4H), 2.83 (d, J=4.6 Hz, 3H), 3.50-3.60 (m, 1H), 3.83-4.05 (m, 3H), 4.11-4.81 (m, 2H), 5.30 (br d, J=8.4 Hz, 1H), 6.48 (br s, 1H), 7.35 (br d, J=7.7 Hz, 2H), 7.41 (dd, J=8.1, 2.0 Hz, 1H), 7.67 (d, J=1.8 Hz, 1H), 7.70 (d, J=8.4 Hz, 1H), 7.99 (d, J=8.8 Hz, 2H), 8.27-8.39 (m, 1H); LCMS (method D): Rt 1.86 min

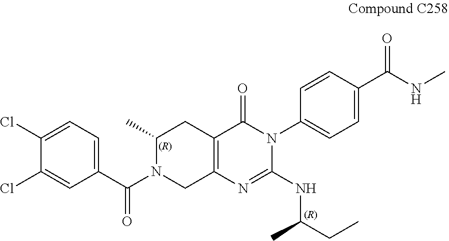

¹H NMR (400 MHz, DMSO-d₆, 81° C.) δ ppm 0.76 (t, J=7.4 Hz, 3H), 1.02 (d, J=6.6 Hz, 3H), 1.18 (d, J=6.8 Hz, 3H), 1.31-1.47 (m, 2H), 2.32 (d, J=16.5 Hz, 1H), 2.53 (br dd, J=16.5, 6.2 Hz, 1H), 2.83 (d, J=4.4 Hz, 3H), 3.86-4.10 (m, 2H), 4.24-4.78 (m, 2H), 4.94 (d, J=7.9 Hz, 1H), 7.34 (d, J=8.4 Hz, 2H), 7.42 (dd, J=8.1, 2.0 Hz, 1H), 7.68 (d, J=2.0 Hz, 1H), 7.70 (d, J=8.1 Hz, 1H), 7.98 (d, J=8.8 Hz, 2H), 8.26-8.38 (m, 1H); LCMS (method B): Rt 2.01 min

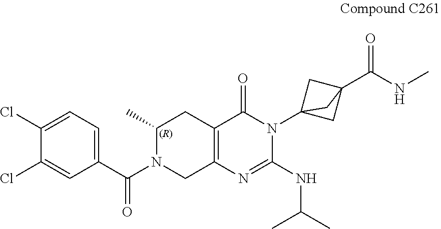

¹H NMR (400 MHz, DMSO-d₆, 81° C.) δ ppm 1.11 (d, J=6.8 Hz, 3H), 1.19 (d, J=6.6 Hz, 3H), 1.20 (d, J=6.6 Hz, 3H), 2.28 (d, J=16.5 Hz, 1H), 2.40-2.49 (m, 1H), 2.59 (d, J=4.6 Hz, 2H), 2.64 (s, 6H), 3.90 (br d, J=18.3 Hz, 1H), 4.09 (dq, J=13.4, 6.6 Hz, 1H), 4.16-4.90 (m, 2H), 5.39 (br d, J=7.3 Hz, 1H), 7.38 (dd, J=8.3, 1.9 Hz, 1H), 7.51-7.60 (m, 1H), 7.64 (d, J=2.0 Hz, 1H), 7.68 (d, J=8.1 Hz, 1H); LCMS (method C): Rt 0.99 min

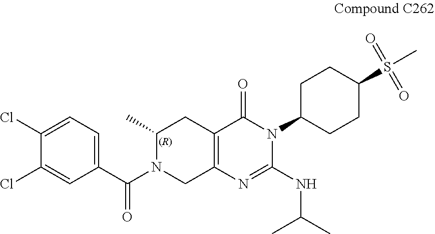

¹H NMR (400 MHz, DMSO-d₆, 100° C.) δ ppm 1.12 (d, J=6.8 Hz, 3H), 1.18 (d, J=6.2 Hz, 3H), 1.19 (d, J=6.2 Hz, 3H), 1.49-1.59 (m, 2H), 1.86-1.99 (m, 2H), 2.31 (d, J=16.3 Hz, 1H), 2.40-2.47 (m, 2H), 2.50-2.63 (m, 3H), 2.95 (s, 3H), 3.33 (dt, J=5.1, 2.6 Hz, 1H), 3.90 (br d, J=18.5 Hz, 1H), 4.22 (dq, J=13.3, 6.7 Hz, 1H), 4.30-4.60 (m, 2H), 5.19 (tt, J=12.7, 5.3 Hz, 1H), 5.67 (br d, J=7.0 Hz, 1H), 7.37 (dd, J=8.1, 2.0 Hz, 1H), 7.63 (d, J=2.0 Hz, 1H), 7.67 (d, J=8.1 Hz, 1H); LCMS (method D): Rt 2.08 min

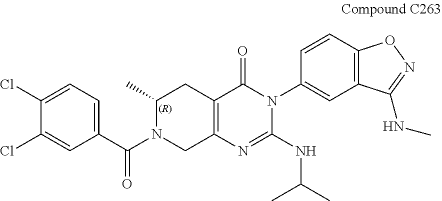

¹H NMR (400 MHz, DMSO-d₆, 100° C.) δ ppm 1.04 (d, J=6.6 Hz, 3H), 1.05 (d, J=6.6 Hz, 3H), 1.19 (d, J=6.8 Hz, 3H), 2.33 (d, J=16.3 Hz, 1H), 2.55 (br dd, J=16.4, 6.1 Hz, 1H), 2.91 (d, J=5.1 Hz, 3H), 4.02 (br d, J=18.7 Hz, 1H), 4.15 (dq, J=13.6, 6.8 Hz, 1H), 4.38-4.63 (m, 2H), 5.21 (br d, J=7.9 Hz, 1H), 6.62 (br s, 1H), 7.32 (br s, 1H), 7.41 (dd, J=8.3, 2.0 Hz, 1H), 7.55 (d, J=8.8 Hz, 1H), 7.61-7.73 (m, 3H); LCMS (method D): Rt 2.05 min

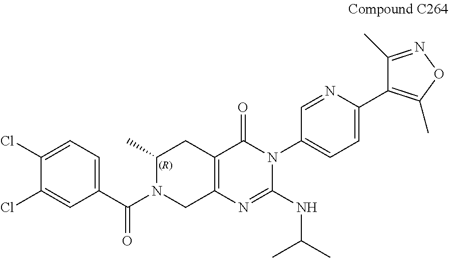

¹H NMR (400 MHz, DMSO-d₆, 81° C.) δ ppm 1.06 (d, J=6.6 Hz, 3H), 1.07 (d, J=6.6 Hz, 3H), 1.19 (d, J=6.8 Hz, 3H), 2.33 (br d, J=16.4 Hz, 1H), 2.45 (s, 3H), 2.51-2.59 (m, 1H), 2.64 (s, 3H), 4.03 (br d, J=17.8 Hz, 1H), 4.18 (dq, J=13.7, 6.7 Hz, 1H), 4.25-4.80 (m, 2H), 5.64 (br d, J=7.7 Hz, 1H), 7.41 (dd, J=8.3, 1.9 Hz, 1H), 7.68 (d, J=2.0 Hz, 1H), 7.69-7.75 (m, 2H), 7.79 (dd, J=8.5, 2.5 Hz, 1H), 8.31 (d, J=2.6 Hz, 1H); LCMS (method C): Rt 1.15 min

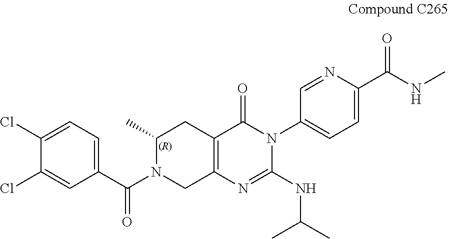

¹H NMR (400 MHz, DMSO-d₆, 81° C.) δ ppm 1.05 (d, J=6.6 Hz, 3H), 1.06 (d, J=6.6 Hz, 3H), 1.19 (d, J=6.8 Hz, 3H), 2.33 (br d, J=16.3 Hz, 1H), 2.54 (br dd, J=16.6, 6.1 Hz, 1H), 2.88 (d, J=5.1 Hz, 3H), 4.03 (br d, J=18.7 Hz, 1H), 4.18 (dq, J=13.6, 6.8 Hz, 1H), 4.24-4.94 (m, 2H), 5.70 (d, J=7.9 Hz, 1H), 7.41 (dd, J=8.1, 2.0 Hz, 1H), 7.67 (d, J=2.0 Hz, 1H), 7.70 (d, J=8.4 Hz, 1H), 7.89 (dd, J=8.3, 2.3 Hz, 1H), 8.17 (dd, J=8.3, 0.6 Hz, 1H), 8.48 (d, J=2.2 Hz, 1H), 8.56-8.66 (m, 1H); LCMS (method C): Rt 1.02 min

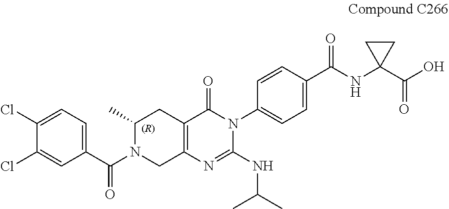

¹H NMR (400 MHz, DMSO-d₆, 81° C.) δ ppm 1.05 (d, J=6.6 Hz, 3H), 1.06 (d, J=6.6 Hz, 3H), 1.11-1.16 (m, 2H), 1.18 (d, J=6.8 Hz, 3H), 1.37-1.43 (m, 2H), 2.33 (br d, J=16.1 Hz, 1H), 2.53 (br dd, J=16.5, 5.9 Hz, 1H), 4.02 (br d, J=18.5 Hz, 1H), 4.14 (dq, J=13.9, 6.7 Hz, 1H), 4.30-4.70 (m, 2H), 5.09 (br d, J=7.7 Hz, 1H), 7.32 (br d, J=8.1 Hz, 2H), 7.42 (dd, J=8.1, 2.0 Hz, 1H), 7.68 (d, J=1.8 Hz, 1H), 7.70 (d, J=8.4 Hz, 1H), 8.00 (d, J=8.8 Hz, 2H), 8.85 (br s, 1H); LCMS (method C): Rt 0.81 min

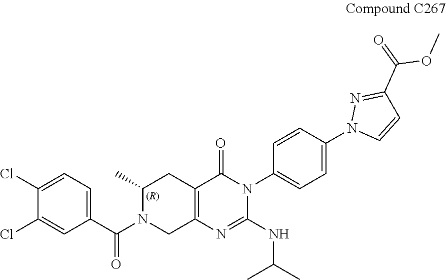

¹H NMR (400 MHz, DMSO-d₆, 100° C.) δ ppm 1.06 (d, J=6.5 Hz, 3H), 1.07 (d, J=6.5, 3H), 1.20 (d, J=6.8 Hz, 3H), 2.35 (d, J=16.5 Hz, 1H), 2.55 (br dd, J=16.3, 5.9 Hz, 1H), 3.87 (s, 3H), 4.02 (br d, J=18.7 Hz, 1H), 4.15 (dq, J=13.4, 6.8 Hz, 1H), 4.38-4.67 (m, 2H), 5.10 (br d, J=7.7 Hz, 1H), 7.00 (d, J=2.4 Hz, 1H), 7.36-7.48 (m, 3H), 7.66 (d, J=1.8 Hz, 1H), 7.69 (d, J=8.1 Hz, 1H), 8.04 (d, J=9.0 Hz, 2H), 8.60 (d, J=2.4 Hz, 1H); LCMS (method D): Rt 2.00 min

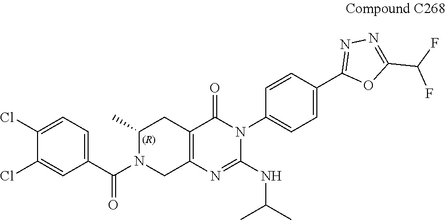

¹H NMR (400 MHz, DMSO-d₆, 100° C.) δ ppm 1.06 (d, J=6.6 Hz, 3H), 1.07 (d, J=6.6 Hz, 3H), 1.19 (d, J=6.8 Hz, 3H), 2.34 (d, J=16.5 Hz, 1H), 2.51-2.60 (m, 1H), 4.03 (br d, J=19.8 Hz, 1H), 4.15 (dq, J=13.4, 6.6 Hz, 1H), 4.51 (br s, 2H), 5.15 (br d, J=7.3 Hz, 1H), 7.41 (dd, J=8.3, 1.9 Hz, 1H), 7.49 (t, J=51.5 Hz, 1H), 7.51-7.56 (m, 2H), 7.66 (d, J=1.8 Hz, 1H), 7.69 (d, J=8.4 Hz, 1H), 8.18-8.23 (m, 2H); LCMS (method C): Rt 1.19 min

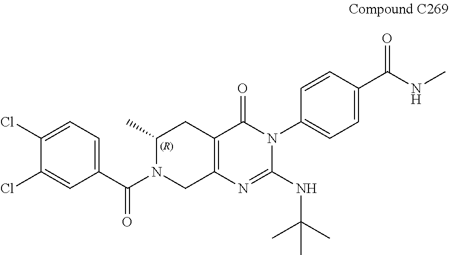

¹H NMR (400 MHz, DMSO-d₆, 81° C.) δ ppm 1.18 (d, J=6.8 Hz, 3H), 1.29 (s, 9H), 2.33 (br d, J=16.3 Hz, 1H), 2.51-2.61 (m, 1H), 2.82 (d, J=4.6 Hz, 3H), 4.02 (br d, J=19.2 Hz, 1H), 4.17 (s, 1H), 4.29-4.93 (m, 2H), 7.39 (d, J=8.4 Hz, 2H), 7.42 (dd, J=8.1, 2.0 Hz, 1H), 7.68 (d, J=1.8 Hz, 1H), 7.71 (d, J=8.4 Hz, 1H), 7.99 (d, J=8.8 Hz, 2H), 8.31-8.40 (m, 1H); LCMS (method C): Rt 1.10 min

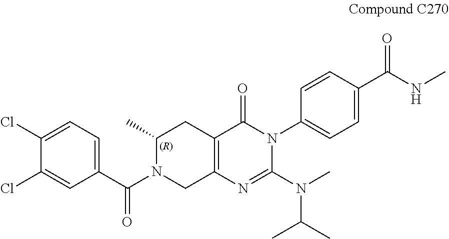

¹H NMR (400 MHz, DMSO-d₆, 81° C.) δ ppm 0.84 (d, J=6.8 Hz, 3H), 0.86 (d, J=6.8 Hz, 3H), 1.18 (d, J=6.8 Hz, 3H), 2.31 (s, 3H), 2.35 (br d, J=16.7 Hz, 1H), 2.55 (br dd, J=16.6, 5.8 Hz, 1H), 2.81 (d, J=4.6 Hz, 3H), 3.90-4.01 (m, 1H), 4.06 (br d, J=18.5 Hz, 1H), 4.27-4.84 (m, 2H), 7.40 (d, J=8.4 Hz, 2H), 7.43 (d, J=2.0 Hz, 1H), 7.68 (d, J=2.0 Hz, 1H), 7.70 (d, J=8.1 Hz, 1H), 7.90 (d, J=8.8 Hz, 2H), 8.24-8.33 (m, 1H); LCMS (method C): Rt 1.05 min

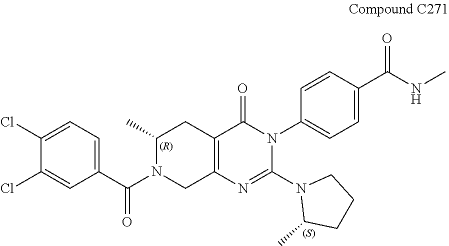

¹H NMR (400 MHz, DMSO-d₆, 81° C.) δ ppm 1.13 (d, J=5.9 Hz, 3H), 1.17 (d, J=6.8 Hz, 3H), 1.21-1.35 (m, 1H), 1.42-1.68 (m, 2H), 1.84-1.96 (m, 1H), 2.24-2.39 (m, 2H), 2.57 (br dd, J=16.4, 6.1 Hz, 1H), 2.81 (d, J=4.6 Hz, 3H), 2.95 (ddd, J=10.2, 7.8, 3.0 Hz, 1H), 3.94 (dq, J=13.5, 6.5 Hz, 1H), 4.05 (br d, J=18.5 Hz, 1H), 4.22-4.89 (m, 2H), 7.24-7.46 (m, 2H), 7.42 (dd, J=8.1, 2.0 Hz, 1H), 7.68 (d, J=1.8 Hz, 1H), 7.71 (d, J=8.4 Hz, 1H), 7.86-7.94 (m, 2H), 8.25-8.37 (m, 1H); LCMS (method C): Rt 1.07 min

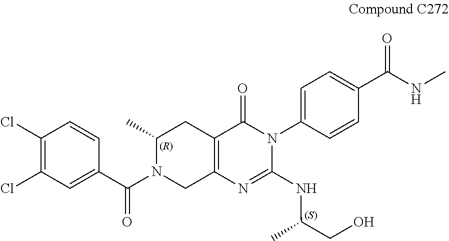

¹H NMR (400 MHz, DMSO-d₆, 81° C.) δ ppm 1.03 (d, J=6.6 Hz, 3H), 1.19 (d, J=6.6 Hz, 3H), 2.33 (br d, J=16.3 Hz, 1H), 2.54 (br dd, J=16.2, 5.8 Hz, 1H), 2.82 (d, J=4.6 Hz, 3H). 3.30 (br d, J=4.8 Hz, 2H), 3.95-4.11 (m, 2H), 4.31-4.67 (m, 2H), 4.96 (d, J=7.7 Hz, 1H), 7.36 (br d, J=8.4 Hz, 2H), 7.42 (dd, J=8.1, 2.0 Hz, 1H), 7.68 (d, J=1.8 Hz, 1H), 7.71 (d, J=8.4 Hz, 1H), 7.98 (d, J=8.8 Hz, 2H), 8.29-8.39 (m, 1H); LCMS (method C): Rt 0.85 min

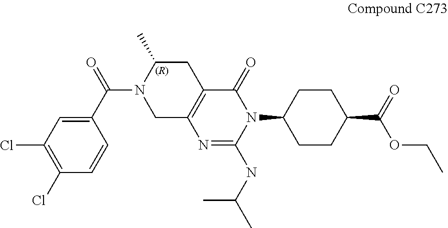

¹H NMR (400 MHz, DMSO-d₆, 100° C.) δ ppm 1.08-1.15 (m, 3H), 1.15-1.27 (m, 9H), 1.48 (br d, J=11.5 Hz, 2H), 1.61-1.72 (m, 2H), 2.10 (br d, J=13.6 Hz, 2H), 2.29 (br d, J=16.3 Hz, 1H), 2.44 (br d, J=6.1 Hz, 1H), 2.50-2.62 (m, 2H), 2.63-2.69 (m, 1H), 3.89 (br d, J=18.3 Hz, 1H), 4.10-4.23 (m, 3H), 4.32-4.50 (m, 2H), 4.52-4.64 (m, 1H), 5.92 (br d, J=6.6 Hz, 1H), 7.37 (dd, J=8.1, 1.8 Hz, 1H), 7.62 (d, J=2.0 Hz, 1H), 7.67 (d, J=8.1 Hz, 1H); LCMS (method D): Rt 2.39 min

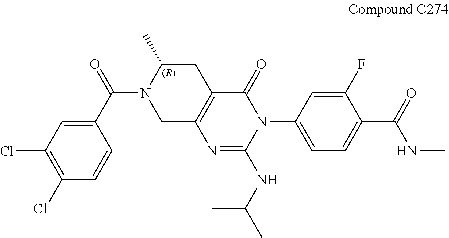

¹H NMR (400 MHz, DMSO-d₆, 100° C.) δ ppm 1.07 (d, J=6.6 Hz, 3H), 1.08 (d, J=6.6 Hz, 3H), 1.18 (d, J=6.8 Hz, 3H), 2.32 (br d, J=16.5 Hz, 1H), 2.53 (br dd, J=16.7, 5.9 Hz, 1H), 2.83 (d, J=4.6 Hz, 3H), 4.01 (br d, J=18.9 Hz, 1H), 4.15 (dq, J=13.7, 6.7 Hz, 1H), 4.28-4.78 (m, 2H), 5.23 (d, J=7.7 H, 1H), 7.15 (dd, J=8.0, 1.4 Hz, 1H), 7.25 (br dd, J=10.9, 1.4 Hz, 1H), 7.40 (dd, J=8.3, 1.9 Hz, 1H), 7.65 (d, J=1.8 Hz, 1H), 7.69 (d, J=8.1 Hz, 1H), 7.78 (br t, J=8.0 Hz, 1H), 7.99 (s, 1H); LCMS (method D): Rt 1.92 min

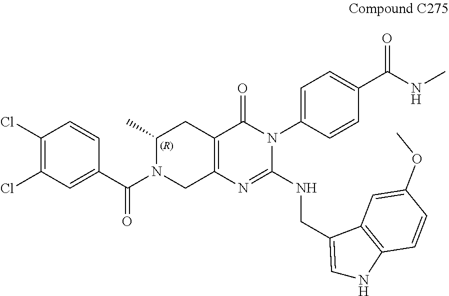

¹H NMR (400 MHz, DMSO-d₆, 81° C.) δ ppm 1.19 (d, J=6.6 Hz, 3H), 2.33 (d, J=16.5 Hz, 1H), 2.54 (dd, J=16.5, 5.9 Hz, 1H), 2.80 (d, J=4.6 Hz, 3H), 3.71 (s, 3H), 4.06 (br d, J=18.1 Hz, 1H), 4.23-4.95 (m, 4H), 5.82 (br t, J=5.7 Hz, 1H), 6.69 (dd, J=8.6, 2.4 Hz, 1H), 7.05 (d, J=2.2 Hz, 1H), 7.11 (br s, 1H), 7.19 (d, J=8.8 Hz, 1H), 7.30 (br d, J=7.3 Hz, 2H), 7.42 (dd, J=8.3, 1.9 Hz, 1H), 7.68 (d, J=2.0 Hz, 1H), 7.70 (d, J=8.4 Hz, 1H), 7.93 (d, J=8.8 Hz, 2H), 8.20-8.35 (m, 1H), 10.48 (br s, 1H); LCMS (method C): Rt 1.00 min

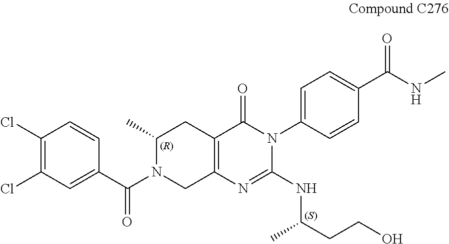

¹H NMR (400 MHz, DMSO-d₆, 81° C.) δ ppm 1.08 (d, J=6.6 Hz, 3H), 1.19 (d, J=6.6 Hz, 3H), 1.44-1.61 (m, 2H), 2.33 (d, J=16.5 Hz, 1H), 2.53 (dd, J=16.3, 5.9 Hz, 1H), 2.82 (d, J=4.6 Hz, 3H), 3.31-3.46 (m, 2H), 4.02 (br d, J=18.5 Hz, 1H), 4.07-4.21 (m, 2H), 4.21-4.92 (m, 2H), 5.47 (d, J=7.7 Hz, 1H), 7.32 (br d, J=8.1 Hz, 2H), 7.42 (dd, J=8.2, 2.0 Hz, 1H), 7.68 (d, J=2.0 Hz, 1H), 7.70 (d, J=8.4 Hz, 1H), 7.97 (d, J=8.8 Hz, 2H), 8.25-8.35 (m, 1H); LCMS (method C): Rt 0.88 min

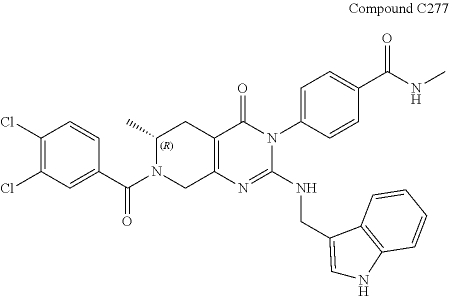

¹H NMR (400 MHz, DMSO-d₆, 81° C.) δ ppm 1.19 (d, J=6.8 Hz, 3H), 2.33 (d, J=16.5 Hz, 1H), 2.54 (dd, J=16.5, 6.1 Hz, 1H), 2.81 (d, J=4.6 Hz, 3H), 4.08 (br d, J=19.4 Hz, 1H), 4.19-5.28 (m, 4H), 5.89 (br t, J=5.5 Hz, 1H), 6.89 (br t, J=7.4 Hz, 1H), 6.99-7.07 (m, 1H), 7.15 (d, J=1.8 Hz, 1H), 7.24-7.36 (m, 3H), 7.42 (dd, J=8.2, 2.0 Hz, 1H), 7.55 (d, J=7.9 Hz, 1H), 7.69 (d, J=2.0 Hz, 1H), 7.71 (d, J=8.4 Hz, 1H), 7.94 (d, J=8.8 Hz, 2H). 8.23-8.37 (m, 1H), 10.65 (br s, 1H); LCMS (method C): Rt 1.02 min

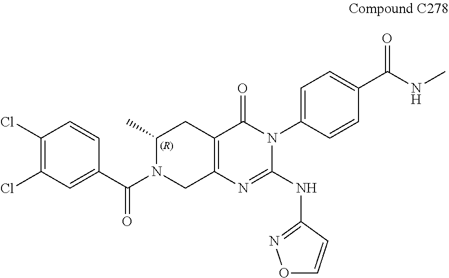

¹H NMR (400 MHz, DMSO-d₆, 81° C.) δ ppm 1.19 (d, J=6.8 Hz, 3H), 2.37 (br d, J=16.5 Hz, 1H), 2.55-2.64 (m, 1H), 2.83 (d, J=4.5 Hz, 3H), 4.08 (br d, J=18.7 Hz, 1H), 4.24-4.80 (m, 2H), 6.71-7.05 (m, 1H), 7.34-7.49 (m, 3H), 7.69 (d, J=2.0 Hz, 1H), 7.70 (d, J=8.4 Hz, 1H), 7.96 (d, J=8.6 Hz, 2H), 8.26-8.36 (m, 1H), 8.63 (s, 1H), 8.44-9.03 (m, 1H); LCMS (method C): Rt 0.90 min

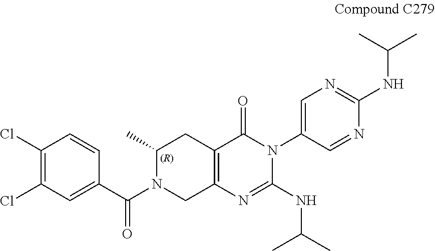

¹H NMR (400 MHz, DMSO-d₆, 101° C.) δ ppm 1.05-1.13 (m, 6H), 1.17 (br d, J=7.0 Hz, 3H), 1.21 (br d, J=6.4 Hz, 6H), 2.32 (br d, J=16.4 Hz, 1H), 2.51-2.58 (m, 1H), 4.00 (br d, J=18.9 Hz, 1H), 4.05-4.19 (m, 2H), 4.36-4.70 (m, 2H), 5.83 (br d, J=6.6 Hz, 1H), 6.90 (br d, J=5.7 Hz, 1H), 7.39 (br d, J=8.1 Hz, 1H), 7.64 (s, 1H), 7.68 (br d, J=8.1 Hz, 1H), 8.05 (s, 2H); LCMS (method D): Rt 2.14 min

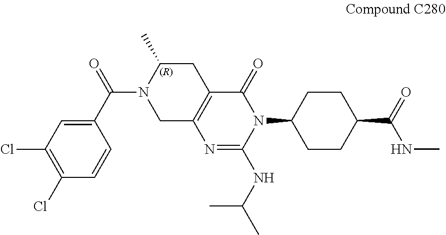

¹H NMR (400 MHz, DMSO-d₆, 100° C.) δ ppm 1.13 (d, J=6.8 Hz, 3H), 1.26 (d, J=6.6 Hz, 3H), 1.27 (d, J=6.6 Hz, 3H), 1.39-1.49 (m, 2H), 1.55-1.65 (m, 2H), 1.86-1.96 (m, 2H), 2.32 (br d, J=16.3 Hz, 1H), 2.50-2.62 (m, 3H), 2.64 (d, J=4.6 Hz, 3H), 3.90 (br d, J=18.7 Hz, 1H), 4.29 (dq, J=13.4, 6.7 Hz, 1H), 4.44 (br s, 2H), 5.25-5.35 (m, 1H), 6.66 (br d, J=7.5 Hz, 1H), 7.38 (dd, J=8.1, 2.0 Hz, 1H), 7.63 (d, J=2.0 Hz, 1H), 7.67 (d, J=8.1 Hz, 1H), 7.71 (br s, 1H); LCMS (method D): Rt 2.16 min

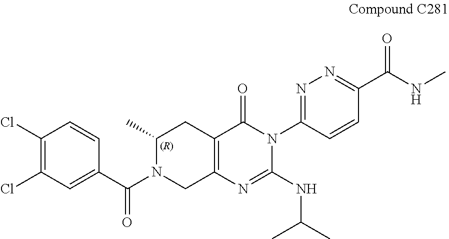

¹H NMR (400 MHz, DMSO-d₆, 150° C.) δ ppm 1.08 (d, J=6.6 Hz, 3H), 1.09 (d, J=6.6 Hz, 3H), 1.20 (d, J=6.6 Hz, 3H), 2.35 (d, J=16.3 Hz, 1H), 2.57 (br dd, J=16.4, 6.1 Hz, 1H), 2.94 (d, J=4.9 Hz, 3H), 4.03 (d, J=18.9 Hz, 1H), 4.17-4.28 (m, 1H), 4.48-4.66 (m, 2H), 5.64 (br d, J=7.3 Hz, 1H), 7.39 (dd, J=8.3, 1.9 Hz, 1H), 7.63 (d, J=1.8 Hz, 1H), 7.66 (d, J=8.1 Hz, 1H), 7.91 (d, J=8.8 Hz, 1H), 8.33 (d, J=8.6 Hz, 1H), 8.74 (s, 1H); LCMS (method D): Rt 1.94 min

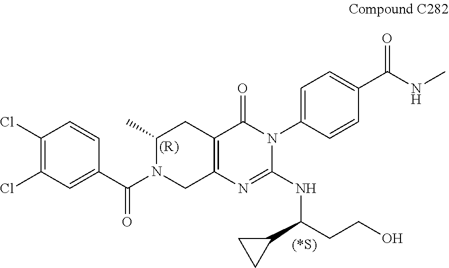

¹H NMR (400 MHz, DMSO-d₆, 81° C.) δ ppm −0.05-0.05 (m, 1H), 0.10-0.27 (m, 3H), 0.63-0.82 (m, 1H), 1.02 (br d, J=6.4 Hz, 3H), 1.41-1.60 (m, 2H), 2.16 (br d, J=16.7 Hz, 1H), 2.35-2.44 (m, 1H), 2.68 (br d, J=3.1 Hz, 3H), 3.17-3.54 (m, 2H), 3.81 (br d, J=19.2 Hz, 1H), 3.98 (br s, 1H), 4.09-4.66 (m, 2H), 5.31 (br d, J=7.5 Hz, 1H), 7.18 (br d, J=7.3 Hz, 2H), 7.26 (br d, J=8.1 Hz, 1H), 7.49-7.60 (m, 2H), 7.83 (br d, J=8.0 Hz, 2H), 8.12-8.22 (m, 1H); LCMS (method D): Rt 1.79 min

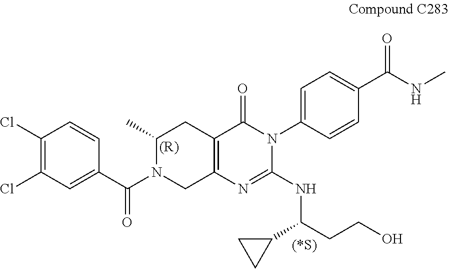

¹H NMR (400 MHz, DMSO-d₆, 81° C.) δ ppm 0.09-0.21 (m, 1H), 0.25-0.44 (m, 3H), 0.83-0.96 (m, 1H), 1.16-1.21 (m, 3H), 1.56-1.78 (m, 2H), 2.32 (br d, J=16.5 Hz, 1H), 2.50-2.58 (m, 1H), 2.83 (d, J=4.6 Hz, 3H), 3.35-3.52 (m, 2H), 3.52-3.65 (m, 1H), 3.97 (br d, J=19.1 Hz, 1H), 4.11 (br t, J=4.8 Hz, 1H), 4.16-4.86 (m, 2H), 5.42 (d, J=8.1 Hz, 1H), 7.33 (br d, J=7.9 Hz, 2H), 7.41 (dd, J=8.1, 2.0 Hz, 1H), 7.67 (d, J=1.8 Hz, 1H), 7.70 (d, J=8.1 Hz, 1H), 7.98 (d, J=8.6 Hz, 2H), 8.25-8.40 (m, 1H); LCMS (method D): Rt 1.81 min

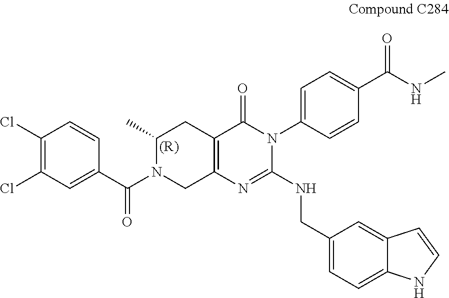

¹H NMR (400 MHz, DMSO-d₆, 81° C.) δ ppm 1.18 (d, J=6.8 Hz, 3H), 2.32 (d, J=16.3 Hz, 1H), 2.50-2.60 (m, 1H), 2.81 (d, J=4.4 Hz, 3H), 4.02 (br d, J=18.9 Hz, 1H), 4.19-4.76 (m, 4H), 6.12 (br t, J=5.6 Hz, 1H), 6.32 (d, J=2.9 Hz, 1H), 6.98 (dd, J=8.4, 1.1 Hz, 1H), 7.23-7.25 (m, 1H), 7.27 (d, J=8.4 Hz, 1H), 7.31-7.39 (m, 3H), 7.40 (dd, J=8.1, 2.0 Hz, 1H), 7.67 (d, J=1.8 Hz, 1H), 7.69 (d, J=8.1 Hz, 1H), 7.97 (d, J=8.8 Hz, 2H), 8.22-8.40 (m, 1H), 10.76 (s, 1H); LCMS (method C): Rt 1.00 min

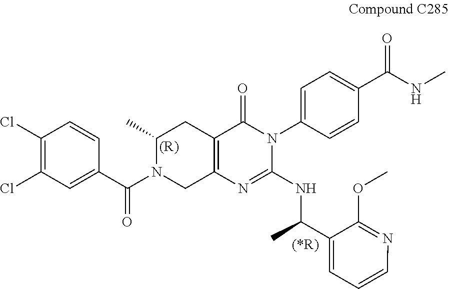

¹H NMR (400 MHz, DMSO-d₆, 81° C.) δ ppm 1.14 (d, J=6.9 Hz, 3H), 1.29 (d, J=6.9 Hz, 3H), 2.32 (d, J=16.3 Hz, 1H), 2.55 (br dd, J=16.7, 6.1 Hz, 1H), 2.84 (d, J=4.5 Hz, 3H), 3.66 (s, 3H), 3.95 (br d, J=18.7 Hz, 1H), 4.50 (br s, 2H), 5.25 (m, 1H), 5.65 (d, J=8.5 Hz, 1H), 6.89 (dd, J=7.3, 5.0 Hz, 1H), 7.35-7.48 (m, 3H), 7.55 (dd, J=7.3, 1.6 Hz, 1H), 7.68 (d, J=2.0 Hz, 1H), 7.70 (d, J=8.1 Hz, 1H), 7.97-8.07 (m, 3H), 8.28-8.38 (m, 1H); LCMS (method D): Rt 2.04 min

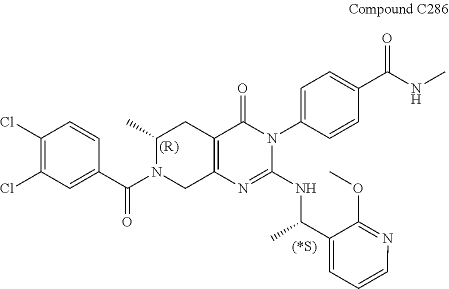

¹H NMR (400 MHz, DMSO-d₆, 81° C.) δ ppm 1.18 (d, J=6.8 Hz, 3H), 1.29 (d, J=6.9 Hz, 3H), 2.32 (d, J=16.3 Hz, 1H), 2.48-2.56 (m, 1H), 2.84 (d, J=4.5 Hz, 3H), 3.68 (s, 3H), 4.01 (br d, J=18.7 Hz, 1H), 4.44 (br s, 2H), 5.24 (m, 1H), 5.63 (d, J=8.5 Hz, 1H), 6.88 (dd, J=7.1, 5.1 Hz, 1H), 7.16-7.51 (m, 3H), 7.53 (dd, J=7.3, 1.6 Hz, 1H), 7.65 (d, J=1.6 Hz, 1H), 7.68 (d, J=8.1 Hz, 1H), 8.00 (dd, J=4.9, 1.6 Hz, 1H), 8.03 (br d, J=8.5 Hz, 2H), 8.28-8.38 (m, 1H); LCMS (method D): Rt 1.99 min

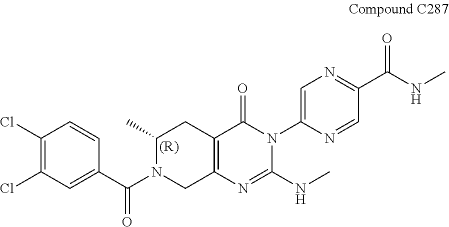

¹H NMR (400 MHz, DMSO-d₆, 81° C.) δ ppm 1.19 (d, J=6.5 Hz, 3H), 2.34 (br d, J=16.3 Hz, 1H), 2.52-2.61 (m, 1H), 2.71 (br s, 3H), 2.89 (d, J=4.9 Hz, 3H), 4.06 (br d, J=19.1 Hz, 1H), 4.31-4.78 (m, 2H), 6.30 (br s, 1H), 7.42 (dd, J=8.2, 2.0 Hz, 1H), 7.68 (d, J=2.0 Hz, 1H), 7.70 (d, J=8.5 Hz, 1H), 8.68-8.75 (m, 1H), 8.78 (d, J=1.2 Hz, 1H), 9.19 (d, J=1.2 Hz, 1H); LCMS (method D): Rt 1.69 min

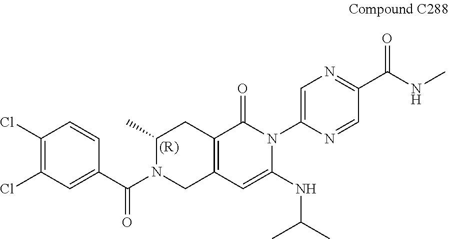

¹H NMR (400 MHz, DMSO-d₆, 81° C.) δ ppm 1.06 (d, J=6.2 Hz, 3H), 1.07 (d, J=6.2 Hz, 3H), 1.19 (d, J=6.8 Hz, 3H), 2.31 (br d, J=16.3 Hz, 1H), 2.39-2.63 (m, 1H), 2.89 (d, J=4.6 Hz, 3H), 4.03 (br d, J=18.9 Hz, 1H), 4.19 (dq, J=13.7, 6.9 Hz, 1H), 4.27-4.88 (m, 2H), 6.01 (br d, J=7.7 Hz, 1H), 7.35-7.47 (m, 1H), 7.61-7.85 (m, 2H), 8.68-8.94 (m, 2H), 9.14-9.29 (m, 1H); LCMS (method C): Rt 1.04 min

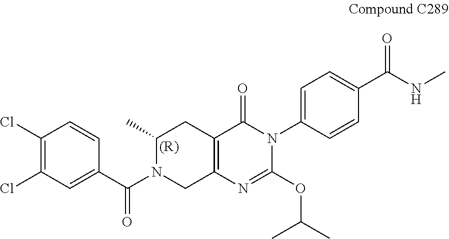

¹H NMR (400 MHz, DMSO-d₆, 81° C.) δ ppm 1.14 (d, J=6.2 Hz, 3H), 1.15 (d, J=6.2 Hz, 3H), 1.19 (d, J=6.8 Hz, 3H), 2.39 (br d, J=16.3 Hz, 1H), 2.55-2.65 (m, 1H), 2.81 (d, J=4.6 Hz, 3H), 4.10 (br d, J=19.3 Hz, 1H), 4.30-4.87 (m, 2H), 5.21 (dt, J=12.4, 6.3 Hz, 1H), 7.30-7.35 (m, 2H), 7.43 (dd, J=8.3, 1.9 Hz, 1H), 7.69 (d, J=2.0 Hz, 1H), 7.71 (d, J=8.4 Hz, 1H), 7.89 (d, J=8.6 Hz, 2H), 8.24-8.33 (m, 1H); LCMS (method C): Rt 1.04 min

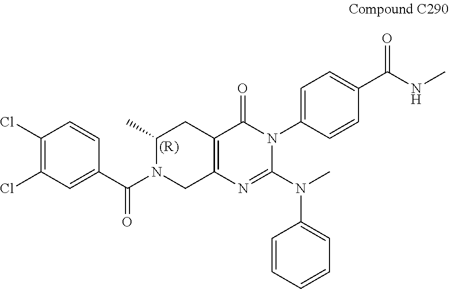

¹H NMR (400 MHz, DMSO-d₆, 81° C.) δ ppm 1.24 (d, J=6.8 Hz, 3H), 2.40 (br d, J=17.0 Hz, 1H), 2.57-2.67 (m, 1H), 2.76 (d, J=4.6 Hz, 3H), 3.15 (s, 3H), 4.19 (br d, J=19.3 Hz, 1H), 4.34-5.18 (m, 2H), 6.62-6.68 (m, 2H), 6.88-6.98 (m, 3H), 6.99-7.10 (m, 2H), 7.45 (dd, J=8.3, 1.9 Hz, 1H), 7.55 (d, J=8.2 Hz, 2H), 7.69-7.75 (m, 2H), 8.04-8.15 (m, 1H); LCMS (method D): Rt 2.00 min

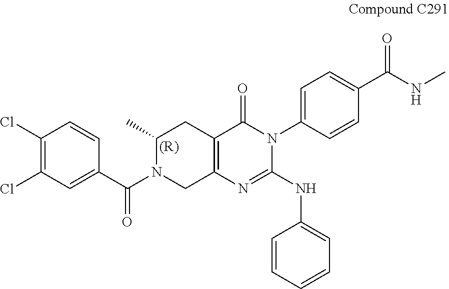

¹H NMR (400 MHz, DMSO-d₆, 81° C.) δ ppm 1.18 (d, J=6.8 Hz, 3H), 2.37 (br d, J=16.7 Hz, 1H), 2.55-2.63 (m, 1H), 2.83 (d, J=4.4 Hz, 3H), 4.00 (br d, J=18.9 Hz, 1H), 4.23-4.86 (m, 2H), 6.99-7.08 (m, 1H), 7.19-7.29 (m, 2H), 7.31-7.52 (m, 6H), 7.67 (d, J=2.0 Hz, 1H), 7.69 (d, J=8.1 Hz, 1H), 7.99 (d, J=8.3 Hz, 2H), 8.26-8.38 (m, 1H); LCMS (method D): Rt 1.93 min

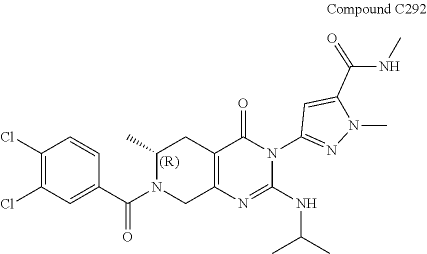

¹H NMR (400 MHz, CDCl₃) δ ppm 1.11-1.19 (m, 6H), 1.21-1.28 (m, 3H), 2.49 (br d, J=16.5 Hz, 1H), 2.62-2.73 (m, 1H), 2.90 (d, J=4.8 Hz, 3H), 3.93-4.35 (m, 6H), 4.89-5.43 (m, 2H), 6.60 (s, 1H), 6.63-6.76 (m, 1H), 7.24-7.28 (m, 1H), 7.49-7.55 (m, 2H); LCMS (method C): Rt 1.03 min

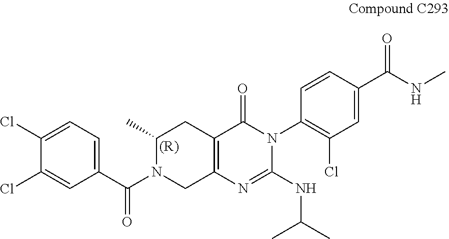

¹H NMR (400 MHz, DMSO-d₆, 81° C.) δ ppm 1.02-1.08 (m, 6H), 1.17 (d, J=6.8 Hz, 3H), 2.29-2.39 (m, 1H), 2.51-2.61 (m, 1H), 2.83 (d, J=4.6 Hz, 3H), 3.92-4.12 (m, 1H), 4.14-4.25 (m, 1H), 4.31-4.84 (m, 2H), 5.52 (d, J=8.1 Hz, 1H), 7.40-7.55 (m, 2H), 7.66-7.72 (m, 2H), 7.92 (dd, J=8.2, 1.9 Hz, 1H), 8.07 (d, J=1.8 Hz, 1H), 8.40-8.50 (m, 1H); LCMS (method B): Rt 2.01 min

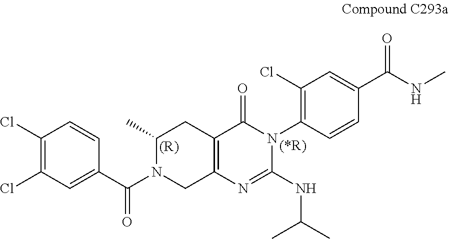

¹H NMR (400 MHz, DMSO-d₆, 80° C.) δ ppm 1.05 (br d, J=6.6 Hz, 3H), 1.06 (br d, J=6.8 Hz, 3H), 1.17 (d, J=6.6 Hz, 3H), 2.32 (br d, J=16.5 Hz, 1H), 2.51-2.59 (m, 1H), 2.83 (d, J=4.4 Hz, 3H), 4.01 (br d, J=18.1 Hz, 1H), 4.20 (dq, J=13.9, 6.9 Hz, 1H), 4.31-4.80 (m, 2H), 5.53 (d, J=8.1 Hz, 1H), 7.44 (dd, J=8.3, 1.9 Hz, 1H), 7.51 (d, J=8.1 Hz, 1H), 7.68-7.72 (m, 2H), 7.92 (dd, J=8.3, 1.9 Hz, 1H), 8.07 (d, J=1.8 Hz, 1H), 8.42-8.516 (m, 1H); LCMS (method C): Rt 1.08 min

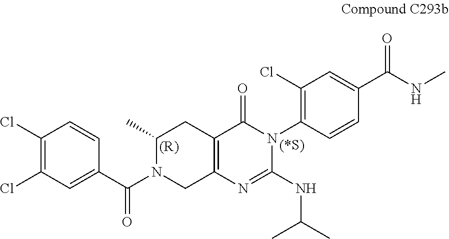

¹H NMR (400 MHz, DMSO-d₆, 80° C.) δ ppm 1.05 (br d, J=6.6 Hz, 3H), 1.06 (br d, J=6.6 Hz, 3H), 1.18 (d, J=6.8 Hz, 3H), 2.33 (d, J=16.5 Hz, 1H), 2.50-2.57 (m, 1H), 2.83 (d, J=4.6 Hz, 3H), 4.04 (br d, J=18.5 Hz, 1H), 4.20 (dq, J=13.4, 6.6 Hz, 1H), 4.28-4.82 (m, 2H), 5.53 (d, J=7.9 Hz, 1H), 7.42 (dd, J=8.4, 2.0 Hz, 1H), 7.47 (d, J=8.4 Hz, 1H), 7.68 (d, J=2.0 Hz, 1H), 7.70 (d, J=8.1 Hz, 1H), 7.93 (dd, J=8.3, 1.9 Hz, 1H), 8.07 (d, J=1.8 Hz, 1H), 8.43-8.50 (m, 1H); LCMS (method D): Rt 2.04 min

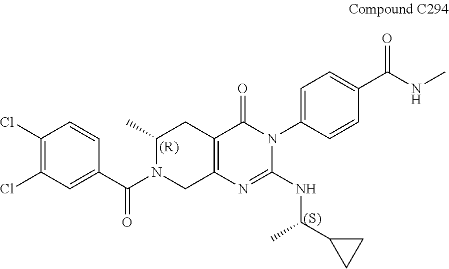

¹H NMR (400 MHz, DMSO-d₆, 81° C.) δ ppm 0.10-0.41 (m, 4H), 0.82-0.93 (m, 1H), 1.11 (d, J=6.6 Hz, 3H), 1.17 (d, J=6.8 Hz, 3H), 2.32 (br d, J=16.3 Hz, 1H), 2.50-2.57 (m, 1H), 2.83 (d, J=4.4 Hz, 3H), 3.44-3.57 (m, 1H), 3.98 (br d, J=17.8 Hz, 1H), 4.18-4.81 (m, 2H), 5.12 (br d, J=7.5 Hz, 1H), 7.35 (br d, J=8.4 Hz, 2H), 7.41 (dd, J=8.3, 1.9 Hz, 1H), 7.67 (d, J=2.0 Hz, 1H), 7.70 (d, J=8.4 Hz, 1H), 7.99 (d, J=8.8 Hz, 2H), 8.29-8.41 (m, 1H); LCMS (method D): Rt 2.04 min

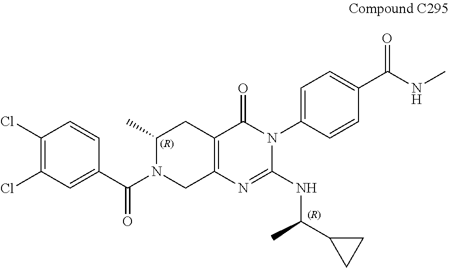

¹H NMR (400 MHz, DMSO-d₆, 81° C.) δ ppm −0.11-0.04 (m, 2H), 0.04-0.20 (m, 2H), 0.63-0.73 (m, 1H), 0.89 (d, J=6.6 Hz, 3H), 0.97 (d, J=6.8 Hz, 3H), 2.11 (br d, J=16.1 Hz, 1H), 2.29-2.37 (m, 1H), 2.62 (d, J=4.4 Hz, 3H), 3.16-3.35 (m, 1H), 3.78 (br d, J=18.5 Hz, 1H), 4.00-4.57 (m, 2H), 4.89 (d, J=7.7 Hz, 1H), 7.14 (br d, J=8.1 Hz, 2H), 7.20 (dd, J=8.3, 1.9 Hz, 1H), 7.46 (d, J=1.8 Hz, 1H), 7.49 (d, J=8.1 Hz, 1H), 7.78 (d, J=8.8 Hz, 2H), 8.09-8.06 (m, 1H); LCMS (method D): Rt 2.05 min

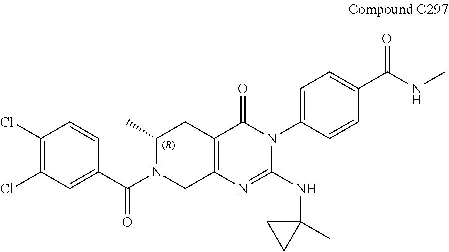

¹H NMR (400 MHz, DMSO-d₆, 81° C.) δ ppm 0.49-0.55 (m, 2H), 0.61-0.66 (m, 2H), 1.19 (d, J=6.9 Hz, 3H), 1.32 (s, 3H), 2.33 (d, J=16.7 Hz, 1H), 2.51-2.59 (m, 1H), 2.82 (d, J=4.5 Hz, 3H), 4.03 (br d, J=17.9 Hz, 1H), 4.23-4.89 (m, 2H), 5.78 (br s, 1H), 7.26 (d, J=8.5 Hz, 2H), 7.42 (dd, J=8.3, 1.8 Hz, 1H), 7.68 (d, J=2.0 Hz, 1H), 7.70 (d, J=8.1 Hz, 1H), 7.94 (d, J=8.5 Hz, 2H), 8.21-8.30 (m, 1H); LCMS (method C): Rt 0.99 min

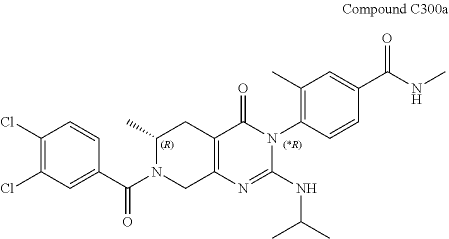

¹H NMR (400 MHz, DMSO-d₆, 81° C.) δ ppm 0.96-1.09 (m, 6H), 1.18 (d, J=6.8 Hz, 3H), 2.04 (s, 3H), 2.34 (d, J=16.3 Hz, 1H), 2.53 (br dd, J=16.3, 6.2 Hz, 1H), 2.82 (d, J=4.6 Hz, 3H), 4.04 (br d, J=18.1 Hz, 1H), 4.16 (dq, J=13.6, 6.8 Hz, 1H), 4.25-4.90 (m, 2H), 5.08 (d, J=7.9 Hz, 1H), 7.21 (d, J=8.1 Hz, 1H), 7.42 (dd, J=8.3, 1.9 Hz, 1H), 7.68 (d, J=2.0 Hz, 1H), 7.70 (d, J=8.4 Hz, 1H), 7.78 (dd, J=8.1, 1.8 Hz, 1H), 7.87 (d, J=1.8 Hz, 1H), 8.22-8.33 (m, 1H); LCMS (method D): Rt 1.94 min

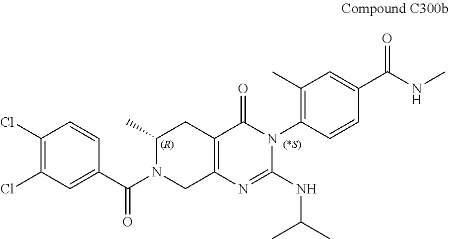

¹H NMR (400 MHz, DMSO-d₆, 81° C.) δ ppm 0.98-1.10 (m, 6H), 1.18 (d, J=6.8 Hz, 3H), 2.04 (s, 3H), 2.33 (d, J=16.5 Hz, 1H), 2.55 (br dd, J=16.5, 5.9 Hz, 1H), 2.82 (d, J=4.4 Hz, 3H), 4.02 (br d, J=17.8 Hz, 1H), 4.10-4.25 (m, 1H), 4.27-4.94 (m, 2H), 5.08 (d, J=7.9 Hz, 1H), 7.24 (d, J=8.1 Hz, 1H), 7.43 (dd, J=8.1, 1.8 Hz, 1H), 7.67-7.73 (m, 2H), 7.78 (dd, J=8.0, 1.9 Hz, 1H), 7.87 (d, J=1.8 Hz, 1H), 8.24-8.33 (m, 1H); LCMS (method D): Rt 1.92 min

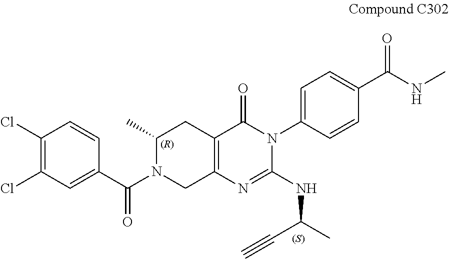

¹H NMR (400 MHz, DMSO-d₆, 81° C.) δ ppm 1.18 (d, J=6.8 Hz, 3H), 1.30 (d, J=6.8 Hz, 3H), 2.33 (d, J=16.5 Hz, 1H), 2.54 (br dd, J=16.5, 5.7 Hz, 1H), 2.83 (d, J=4.6 Hz, 3H), 2.94 (d, J=2.2 Hz, 1H), 4.02 (br d, J=18.9 Hz, 1H), 4.23-4.78 (m, 2H), 4.85-4.95 (m, 1H), 5.91 (d, J=7.9 Hz, 1H), 7.32 (br d, J=8.4 Hz, 2H), 7.42 (dd, J=8.1, 2.0 Hz, 1H), 7.68 (d, J=1.8 Hz, 1H), 7.70 (d, J=8.1 Hz, 1H), 7.98 (d, J=8.8 Hz, 2H), 8.27-8.37 (m, 1H); LCMS (method C): Rt 0.98 min

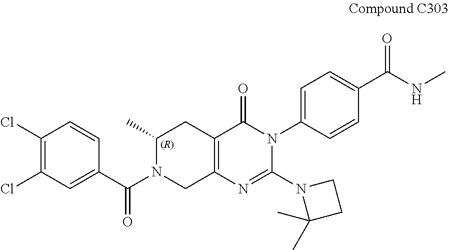

¹H NMR (400 MHz, DMSO-d₆, 81° C.) δ ppm 1.17 (d, J=6.6 Hz, 3H), 1.50 (s, 3H), 1.52 (s, 3H), 1.76 (t, J=7.7 Hz, 2H), 2.30 (d, J=16.3 Hz, 1H), 2.49-2.59 (m, 1H), 2.81 (d, J=4.6 Hz, 3H), 2.86-3.02 (m, 2H), 3.96 (br d, J=19.3 Hz, 1H), 4.17-4.78 (m, 2H), 7.31-7.46 (m, 3H), 7.67 (d, J=2.0 Hz, 1H), 7.70 (d, J=8.1 Hz, 1H), 7.91 (d, J=8.7 Hz, 2H), 8.25-8.34 (m, 1H); LCMS (method C): Rt 1.07 min

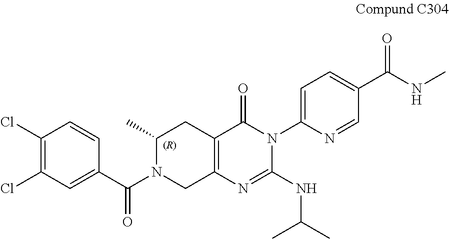

¹H NMR (400 MHz, DMSO-d₆, 81° C.) δ ppm 1.01-1.11 (m, 6H), 1.18 (d, J=6.9 Hz, 3H), 2.32 (d, J=16.3 Hz, 1H), 2.54 (dd, J=16.3, 6.1 Hz, 1H), 2.85 (d, J=4.5 Hz, 3H), 4.02 (br d, J=18.7 Hz, 1H), 4.17 (dq, J=13.7, 6.7 Hz, 1H), 4.25-4.87 (m, 2H), 5.53 (d, J=7.7 Hz, 1H), 7.42 (dd, J=8.3, 1.8 Hz, 1H), 7.51 (dd, J=8.1, 0.8 Hz, 1H), 7.67 (d, J=1.6 Hz, 1H), 7.70 (d, J=8.1 Hz, 1H), 8.35 (dd, J=8.1, 2.4 Hz, 1H), 8.45-8.55 (m, 1H), 9.01 (dd, J=2.4, 0.8 Hz, 1H); LCMS (method C): Rt 0.98 min

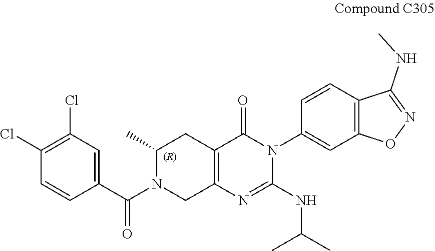

¹H NMR (400 MHz, DMSO-d₆, 100° C.) δ ppm 1.00-1.07 (m, 6H), 1.19 (d, J=6.8 Hz, 3H), 2.33 (br d, J=16.3 Hz, 1H), 2.54 (br dd, J=16.5, 5.9 Hz, 1H), 2.93 (d, J=4.8 Hz, 3H), 4.02 (br d, J=18.9 Hz, 1H), 4.15 (dq, J=13.6, 6.8 Hz, 1H), 4.35-4.68 (m, 2H), 5.15 (d, J=7.9 Hz, 1H), 6.74 (q, J=4.9 Hz, 1H), 7.05 (br d, J=7.9 Hz, 1H), 7.36-7.44 (m, 2H), 7.66 (d, J=2.0 Hz, 1H), 7.69 (d, J=8.1 Hz, 1H), 7.89 (d, J=8.4 Hz, 1H); LCMS (method D): Rt 1.99 min

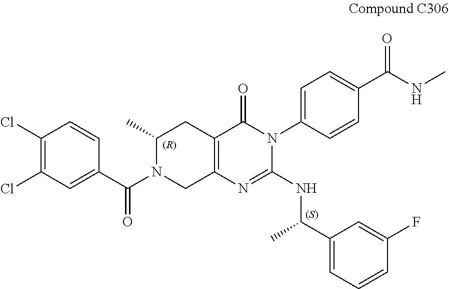

¹H NMR (400 MHz, DMSO-d₆, 81° C.) δ ppm 1.17 (d, J=6.8 Hz, 3H), 1.35 (d, J=7.0 Hz, 3H), 2.30 (d, J=16.3 Hz, 1H), 2.50-2.56 (m, 1H), 2.83 (d, J=4.6 Hz, 3H), 3.98 (br d, J=18.8 Hz, 1H), 4.12-4.78 (m, 2H), 5.22 (quin, J=7.3 Hz, 1H), 5.84 (d, J=7.7 Hz, 1H), 6.90-7.47 (m, 7H), 7.64 (d, J=2.0 Hz, 1H), 7.67 (d, J=8.4 Hz, 1H), 7.96-8.03 (m, 2H), 8.29-8.39 (m, 1H); LCMS (method C): Rt 1.10 min

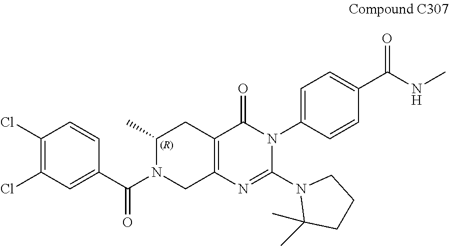

¹H NMR (400 MHz, DMSO-d₆, 81° C.) δ ppm 1.17 (d, J=6.8 Hz, 3H), 1.51 (s, 3H), 1.53 (s, 3H), 1.54-1.63 (m, 4H), 2.32 (d, J=16.7 Hz, 1H), 2.36-2.45 (m, 1H), 2.49-2.66 (m, 2H), 2.81 (d, J=4.9 Hz, 3H), 4.02 (br d, J=18.3 Hz, 1H), 4.24-4.99 (m, 2H), 7.32 (d, J=8.5 Hz, 2H), 7.41 (dd, J=8.2, 2.0 Hz, 1H), 7.67 (d, J=2.0 Hz, 1H), 7.69 (d, J=8.1 Hz, 1H), 7.88 (d, J=9.0 Hz, 2H), 8.19-8.30 (m, 1H); LCMS (method D): Rt 2.13 min

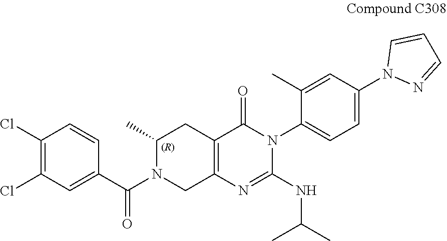

¹H NMR (400 MHz, DMSO-d₆, 80° C.) δ ppm 1.02-1.11 (m, 6H), 1.19 (d, J=6.8 Hz, 3H), 2.07 (s, 3H), 2.30-2.40 (m, 1H), 2.51-2.62 (m, 1H), 3.97-4.11 (m, 1H), 4.19 (dq, J=13.8, 6.7 Hz, 1H), 4.29-4.82 (m, 2H), 5.24 (br d, J=7.9 Hz, 1H), 6.55 (t, J=2.1 Hz, 1H), 7.22-7.29 (m, 1H), 7.41-7.46 (m, 1H), 7.67-7.73 (m, 2H), 7.76 (d, J=1.5 Hz, 1H), 7.81 (dd, J=8.6, 2.2 Hz, 1H), 7.91 (d, J=2.2 Hz, 1H), 8.49 (d, J=2.4 Hz, 1H); LCMS (method C): Rt 1.21 min

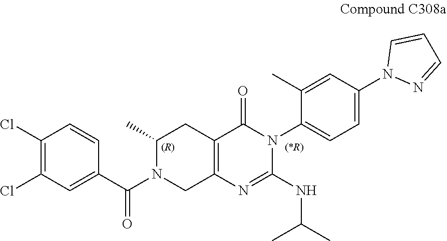

¹H NMR (400 MHz, DMSO-d₆, 80° C.) δ ppm 1.06 (d, J=6.6 Hz, 3H), 1.06 (br d, J=6.6 Hz, 3H), 1.19 (d, J=6.8 Hz, 3H), 2.07 (s, 3H), 2.36 (d, J=16.3 Hz, 1H), 2.55 (br dd, J=16.3, 5.7 Hz, 1H), 4.05 (br d, J=18.7 Hz, 1H), 4.19 (dq, J=13.2, 6.6 Hz, 1H), 4.26-4.90 (m, 2H), 5.24 (d, J=8.1 Hz, 1H), 6.55 (dd, J=2.4, 1.8 Hz, 1H), 7.24 (d, J=8.6 Hz, 1H), 7.43 (dd, J=8.1, 2.0 Hz, 1H), 7.67-7.73 (m, 2H), 7.76 (d, J=1.5 Hz, 1H), 7.81 (dd, J=8.4, 2.4 Hz, 1H), 7.91 (d, J=2.2 Hz, 1H), 8.49 (d, J=2.4 Hz, 1H); LCMS (method H): Rt 2.20 min

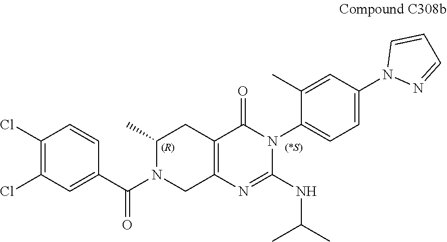

¹H NMR (400 MHz, DMSO-d₆, 80° C.) δ ppm 1.06 (br d, J=6.8 Hz, 3H), 1.06 (br d, J=6.4 Hz, 3H), 1.19 (d, J=6.8 Hz, 3H), 2.06 (s, 3H), 2.34 (br d, J=16.3 Hz, 1H), 2.57 (br dd, J=16.5, 5.9 Hz, 1H), 4.03 (br d, J=18.5 Hz, 1H), 4.19 (dq, J=13.9, 6.8 Hz, 1H), 4.29-4.91 (m, 2H), 5.23 (d, J=8.1 Hz, 1H), 6.55 (dd, J=2.4, 1.8 Hz, 1H), 7.27 (d, J=8.6 Hz, 1H), 7.44 (dd, J=8.1, 2.0 Hz, 1H), 7.68-7.72 (m, 2H), 7.76 (d, J=1.5 Hz, 1H), 7.80 (dd, J=8.5, 2.3 Hz, 1H), 7.91 (d, J=2.4 Hz, 1H), 8.49 (d, J=2.6 Hz, 1H); LCMS (method H): Rt 2.19 min

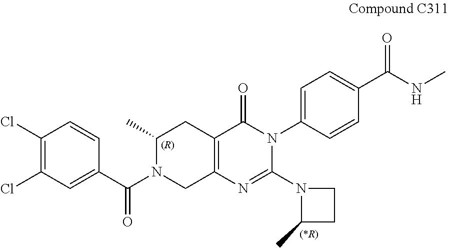

¹H NMR (400 MHz, DMSO-d₆, 81° C.) δ ppm 1.19 (d, J=6.5 Hz, 3H), 1.27 (d, J=6.5 Hz, 3H), 1.58 (ddt, J=10.9, 9.1, 6.9 Hz, 1H), 2.11 (dtd, J=10.8, 8.8, 5.0 Hz, 1H), 2.34 (d, J=16.3 Hz, 1H), 2.53 (br dd, J=16.7, 6.1 Hz, 1H), 2.69 (td, J=8.7, 5.1 Hz, 1H), 2.82 (d, J=4.8 Hz, 3H), 3.33-3.46 (m, 1H), 3.94-4.10 (m, 1H), 4.31 (dq, J=13.5, 6.6 Hz, 1H), 4.36-4.77 (m, 2H), 7.31-7.49 (m, 3H), 7.67 (d, J=1.6 Hz, 1H), 7.69 (d, J=8.1 Hz, 1H), 7.91 (d, J=9.0 Hz, 2H), 8.19-8.35 (m, 1H); LCMS (method D): Rt 1.93 min

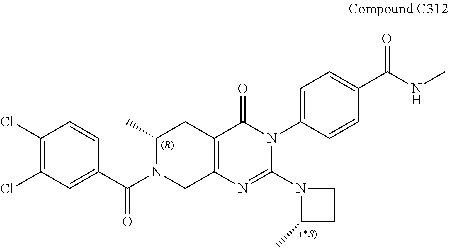

¹H NMR (400 MHz, DMSO-d₆, 81° C.) δ ppm 1.17 (d, J=6.5 Hz, 3H), 1.27 (d, J=6.1 Hz, 3H), 1.57 (ddt, J=10.7, 9.1, 7.0 Hz, 1H), 2.09 (dtd, J=10.8, 8.8, 5.1 Hz, 1H), 2.34 (d, J=16.7 Hz, 1H), 2.56 (br dd, J=16.5, 5.9 Hz, 1H), 2.72 (td, J=8.7, 5.1 Hz, 1H), 2.82 (d, J=4.5 Hz, 3H), 3.32-3.45 (m, 1H), 4.00 (br d, J=19.1 Hz, 1H), 4.23-4.33 (m, 1H), 4.36-4.70 (m, 2H), 7.30-7.49 (m, 3H), 7.67 (d, J=2.0 Hz, 1H), 7.70 (d, J=8.1 Hz, 1H), 7.91 (d, J=8.5 Hz, 2H), 8.20-8.32 (m, 1H); LCMS (method D): Rt 1.94 min

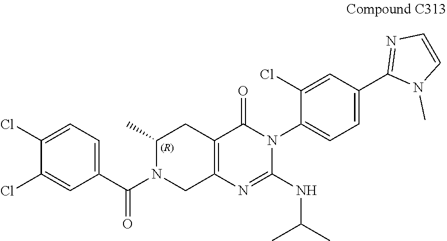

¹H NMR (400 MHz, DMSO-d₆, 80° C.) δ ppm 1.05-1.11 (m, 6H), 1.19 (d, J=6.8 Hz, 3H), 2.28-2.38 (m, 1H), 2.51-2.59 (m, 1H), 3.85 (s, 3H), 3.98-4.11 (m, 1H), 4.15-4.26 (m, 1H), 4.30-4.78 (m, 2H), 5.53 (d, J=7.9 Hz, 1H), 7.04 (d, J=1.1 Hz, 1H), 7.28 (d, J=1.1 Hz, 1H), 7.40-7.52 (m, 2H), 7.67-7.72 (m, 2H), 7.80-7.84 (m, 1H), 7.96 (d, J=1.8 Hz, 1H); LCMS (method B): Rt 2.10 min

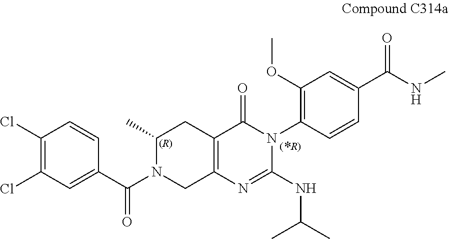

¹H NMR (400 MHz, DMSO-d₆, 81° C.) δ ppm 0.97-1.08 (m, 6H), 1.17 (d, J=6.5 Hz, 3H), 2.30 (br d, J=16.7 Hz, 1H), 2.50-2.59 (m, 1H), 2.83 (br d, J=4.5 Hz, 3H), 3.80 (s, 3H), 3.99 (br d, J=17.9 Hz, 1H), 4.14 (dq, J=13.5, 6.8 Hz, 1H), 4.24-4.81 (m, 2H), 5.01-5.17 (m, 1H), 7.21-7.29 (m, 1H), 7.38-7.46 (m, 1H), 7.50-7.58 (m, 1H), 7.63 (s, 1H), 7.66-7.73 (m, 2H), 8.30 (br s, 1H); LCMS (method D): Rt 2.00 min

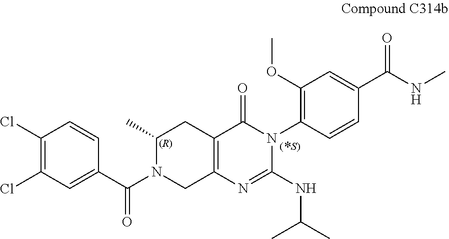

¹H NMR (400 MHz, DMSO-d₆, 81° C.) δ ppm 1.03 (d, J=6.5 Hz, 6H), 1.18 (d, J=6.9 Hz, 3H), 2.31 (br d, J=16.3 Hz, 1H), 2.50-2.57 (m, 1H), 2.84 (d, J=4.5 Hz, 3H), 3.81 (s, 3H), 4.02 (br d, J=18.7 Hz, 1H), 4.14 (dq, J=13.5, 6.7 Hz, 1H), 4.28-4.72 (m, 2H), 5.10 (br s, 1H), 7.19-7.27 (m, 1H), 7.38-7.46 (m, 1H), 7.52-7.59 (m, 1H), 7.61-7.75 (m, 3H), 8.31 (br s, 1H); LCMS (method D): Rt 1.95 min

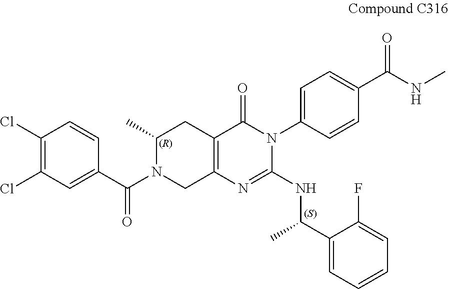

¹H NMR (400 MHz, DMSO-d₆, 81° C.) δ ppm 1.17 (d, J=6.5 Hz, 3H), 1.34 (d, J=6.9 Hz, 3H), 2.30 (d, J=16.7 Hz, 1H), 2.46-2.54 (m, 1H), 2.84 (d, J=4.5 Hz, 3H), 3.97 (br d, J=18.7 Hz, 1H), 4.17-4.65 (m, 2H), 5.38 (quin, J=7.2 Hz, 1H), 5.63 (d, J=7.7 Hz, 1H), 6.99-7.12 (m, 2H), 7.14-7.30 (m, 2H), 7.31-7.43 (m, 3H), 7.62 (d, J=2.0 Hz, 1H), 7.67 (d, J=8.1 Hz, 1H), 7.99-8.04 (m, 2H), 8.27-8.36 (m, 1H); LCMS (method C): Rt 1.10 min

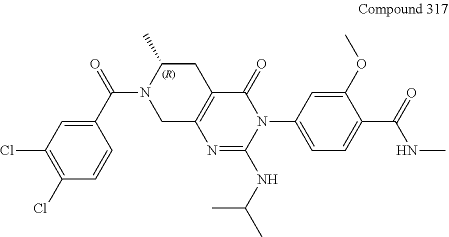

¹H NMR (400 MHz, DMSO-d₆, 100° C.) d ppm 1.07 (d, J=6.6 Hz, 3H), 1.05 (d, J=6.6 Hz, 3H), 1.19 (d, J=6.6 Hz, 3H), 2.34 (br d, J=16.5 Hz, 1H), 2.54 (dd, J=16.5, 5.9 Hz, 1H), 2.84 (d, J=4.8 Hz, 3H), 3.88 (s, 3H), 4.01 (br d, J=18.7 Hz, 1H), 4.05-4.18 (m, 1H), 4.38-4.63 (m, 2H), 5.01 (br d, J=7.7 Hz, 1H), 6.90 (dd, J=8.1, 1.5 Hz, 1H), 7.05 (d, J=1.5 Hz, 1H), 7.40 (dd, J=8.1, 2.0 Hz, 1H), 7.65 (d, J=2.0 Hz, 1H), 7.69 (d, J=8.4 Hz, 1H), 7.89 (d, J=7.9 Hz, 1H), 7.92-7.99 (m, 1H); LCMS (method C): Rt 1.05 min

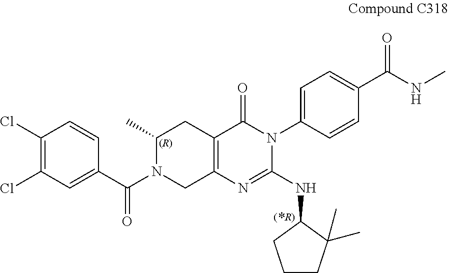

¹H NMR (400 MHz, DMSO-d₆, 81° C.) δ ppm 0.58 (s, 3H), 0.95 (s, 3H), 1.18 (d, J=6.5 Hz, 3H), 1.28-1.54 (m, 5H), 1.99 (dq, J=13.4, 6.8 Hz, 1H), 2.34 (d, J=16.7 Hz, 1H), 2.55 (br dd, J=16.3, 6.1 Hz, 1H), 2.83 (d, J=4.9 Hz, 3H), 3.90-4.10 (m, 2H), 4.28 (d, J=8.5 Hz, 1H), 4.35-4.80 (m, 2H), 7.37-7.45 (m, 3H), 7.67 (d, J=1.6 Hz, 1H), 7.70 (d, J=8.1 Hz, 1H), 8.01 (d, J=8.5 Hz, 2H), 8.26-8.38 (m, 1H); LCMS (method D): Rt 2.16 min

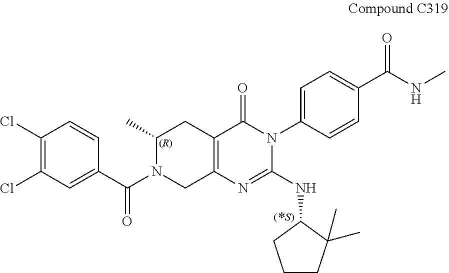

¹H NMR (400 MHz, DMSO-d₆, 81° C.) δ ppm 0.58 (s, 3H), 0.95 (s, 3H), 1.18 (d, J=6. Hz, 3H), 1.28-1.54 (m, 5H), 1.88-2.04 (m, 1H), 2.33 (d, J=16.3 Hz, 1H), 2.55 (br dd, J=16.5, 5.9 Hz, 1H), 2.83 (d, J=4.5 Hz, 3H), 3.88-4.13 (m, 2H), 4.17-4.86 (m, 3H), 7.35-7.46 (m, 3H), 7.67 (d, J=1.6 Hz, 1H), 7.69 (d, J=8.1 Hz, 1H), 8.01 (d, J=8.5 Hz, 2H), 8.27-8.38 (m, 1H); LCMS (method D): Rt 2.14 min

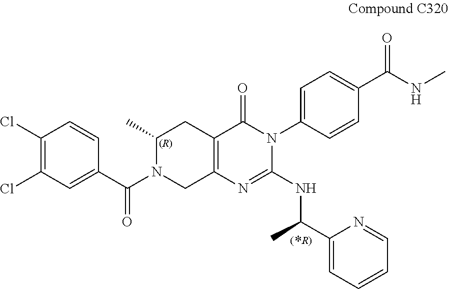

¹H NMR (400 MHz, DMSO-d₆, 81° C.) δ ppm 1.20 (d, J=6.9 Hz, 3H), 1.37 (d, J=6.9 Hz, 3H), 2.35 (br d, J=16.3 Hz, 1H), 2.50-2.60 (m, 1H), 2.84 (d, J=4.5 Hz, 3H), 4.03 (br d, J=18.7 Hz, 1H), 4.18-4.81 (m, 2H), 5.09-5.24 (m, 1H), 6.03 (br d, J=6.9 Hz, 1H), 7.18 (dd, J=6.9, 5.3 Hz, 1H), 7.25-7.52 (m, 4H), 7.63-7.73 (m, 3H), 8.02 (br d, J=8.6 Hz, 2H), 8.25-8.38 (m, 2H); LCMS (method D): Rt 1.91 min

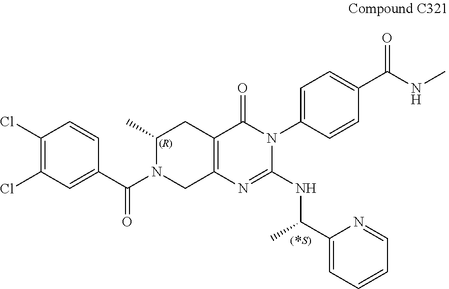

¹H NMR (400 MHz, DMSO-d₆, 81° C.) δ ppm 1.16 (br d, J=6.5 Hz, 3H), 1.37 (br d, J=6.9 Hz, 3H), 2.34 (br d, J=16.7 Hz, 1H), 2.51-2.62 (m, 1H), 2.78-2.89 (m, 3H), 3.99 (br d, J=18.7 Hz, 1H), 4.18-4.87 (m, 2H), 5.10-5.25 (m, 1H), 6.05 (br d, J=7.3 Hz, 1H), 7.11-7.24 (m, 1H), 7.30-7.48 (m, 4H), 7.61-7.76 (m, 3H), 8.02 (br d, J=8.1 Hz, 2H), 8.20-8.39 (m, 2H); LCMS (method D): Rt 1.91 min

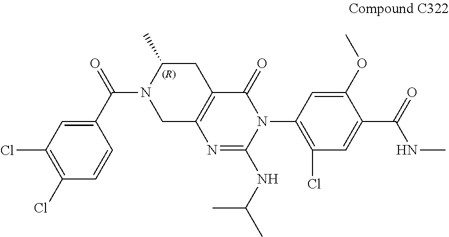

¹H NMR (400 MHz, DMSO-d₆, 125° C.) d ppm 1.01-1.13 (m, 6H), 1.13-1.22 (m, 3H), 2.30-2.43 (m, 1H), 2.49-2.61 (m, 1H), 2.84 (br s, 3H), 3.85-3.92 (m, 3H), 3.94-4.08 (m, 1H), 4.12-4.23 (m, 1H), 4.41-4.61 (m, 2H), 5.23 (br s, 1H), 7.18-7.24 (m, 1H), 7.35-7.43 (m, 1H), 7.60-7.70 (m, 2H), 7.89 (br s, 1H), 7.93 (br s, 1H); LCMS (method D): Rt 2.01 min

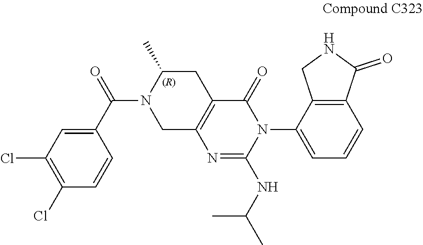

¹H NMR (400 MHz, DMSO-d₆, 125° C.) d ppm 1.05 (dt, J=6.5, 2.1 Hz, 6H), 1.19 (dd, J=6.8, 2.4 Hz, 3H), 2.31-2.38 (m, 1H), 2.50-2.63 (m, 1H), 3.17-4.20 (m, 4H), 4.40-4.63 (m, 2H), 5.26 (br d, J=7.4 Hz, 1H), 7.38-7.42 (m, 1H), 7.42-7.47 (m, 1H), 7.61-7.66 (m, 2H), 7.68 (d, J=8.1 Hz, 1H), 7.79 (d, J=7.3 Hz, 1H), 8.14 (s, 1H); LCMS (method C): Rt 0.99 min

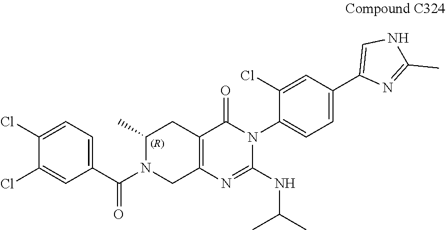

¹H NMR (400 MHz, DMSO-d₆, 80° C.) δ ppm 1.04-1.10 (m, 6H), 1.17 (d, J=6.8 Hz, 3H), 2.27-2.37 (m, 1H), 2.50-2.58 (m, 1H), 3.94-4.10 (m, 1H), 4.15-4.26 (m, 1H), 4.29-4.82 (m, 2H), 5.66-5.71 (m, 1H), 7.31-7.45 (m, 2H), 7.66-7.72 (m, 3H), 7.89-7.92 (m, 1H); LCMS (method C): 1.27 min

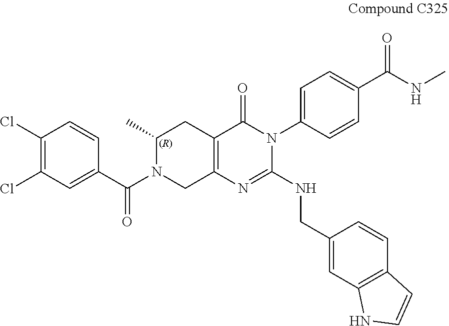

¹H NMR (400 MHz, DMSO-d₆, 81° C.) δ ppm 1.17 (d, J=6.8 Hz, 3H), 2.32 (d, J=16.5 Hz, 1H), 2.54 (br dd, J=16.5, 5.9 Hz, 1H), 2.82 (d, J=4.4 Hz, 3H), 4.00 (br d, J=18.7 Hz, 1H), 4.26-4.74 (m, 4H), 6.22 (br t, J=5.6 Hz, 1H), 6.34 (ddd, J=2.9, 1.9, 0.9 Hz, 1H), 6.89 (dd, J=8.0, 1.0 Hz, 1H), 7.21-7.24 (m, 1H), 7.26 (s, 1H), 7.33-7.43 (m, 4H), 7.65-7.70 (m, 2H), 7.98 (d, J=8.8 Hz, 2H), 8.24-8.39 (m, 1H), 10.76 (br s, 1H); LCMS (method C): Rt 1.03 min

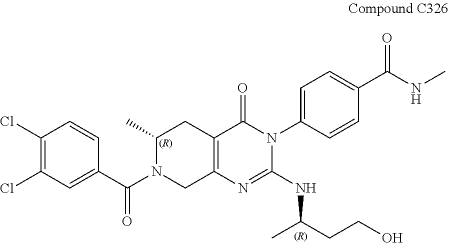

¹H NMR (400 MHz, DMSO-d₆, 81° C.) δ ppm 1.07 (br d, J=6.4 Hz, 3H), 1.18 (br d, J=6.6 Hz, 3H), 1.44-1.62 (m, 2H), 2.32 (br d, J=16.1 Hz, 1H), 2.51-2.59 (m, 1H), 2.82 (d, J=4.6 Hz, 3H), 3.32-3.48 (m, 2H), 3.94-4.05 (m, 1H), 4.07-4.20 (m, 2H), 4.27-4.76 (m, 2H), 5.51 (br d, J=7.3 Hz, 1H), 7.32 (br d, J=7.9 Hz, 2H), 7.42 (dd, J=8.1, 1.8 Hz, 1H), 7.65-7.74 (m, 2H), 7.97 (br d, J=8.6 Hz, 2H), 8.27-8.34 (m, 1H); LCMS (method C): Rt 0.77 min

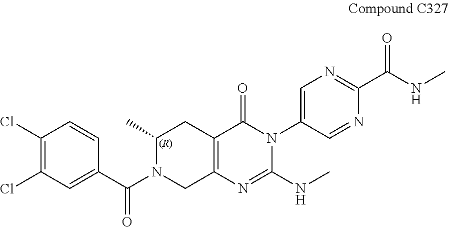

¹H NMR (400 MHz, DMSO-d₆, 81° C.) δ ppm 1.19 (d, J=6.8 Hz, 3H), 2.34 (d, J=16.3 Hz, 1H), 2.55 (br dd, J=16.7, 6.2 Hz, 1H), 2.71 (d, J=4.2 Hz, 3H), 2.88 (d, J=4.8 Hz, 3H), 4.06 (br d, J=18.7 Hz, 1H), 4.26-4.97 (m, 2H), 6.33-6.47 (m, 1H), 7.42 (dd, J=8.1, 2.0 Hz, 1H), 7.68 (d, J=1.8 Hz, 1H), 7.71 (d, J=8.1 Hz, 1H), 8.65-8.79 (m, 1H), 8.91 (s, 2H); LCMS (method D): Rt 1.64 min

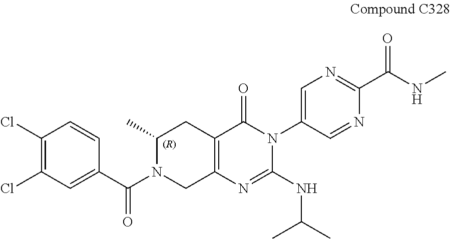

¹H NMR (400 MHz, DMSO-d₆, 81° C.) δ ppm 1.07 (d, J=6.5 Hz, 3H), 1.08 (d, J=6.5 Hz, 3H), 1.19 (d, J=6.5 Hz, 3H), 2.33 (d, J=16.7 Hz, 1H), 2.55 (br dd, J=16.3, 6.1 Hz, 1H), 2.88 (d, J=4.9 Hz, 3H), 4.03 (br d, J=18.3 Hz, 1H), 4.18 (dq, J=13.9, 6.8 Hz, 1H), 4.32-4.77 (m, 2H), 6.09 (br d, J=7.7 Hz, 1H), 7.41 (dd, J=8.3, 1.8 Hz, 1H), 7.67 (d, J=1.6 Hz, 1H), 7.70 (d, J=8.1 Hz, 1H), 8.64-8.74 (m, 1H), 8.87 (s, 2H); LCMS (method C): Rt 0.96 min

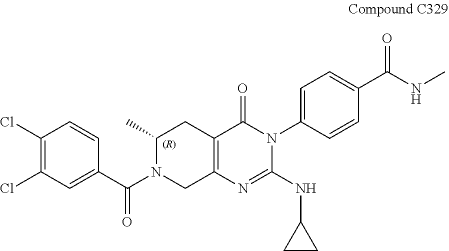

¹H NMR (400 MHz, DMSO-d₆, 100° C.) δ ppm 0.39-0.44 (m, 2H), 0.55-0.61 (m, 2H), 1.19 (d, J=6.8 Hz, 3H), 2.34 (d, J=16.5 Hz, 1H), 2.51-2.59 (m, 1H), 2.71 (tq, J=7.1, 3.8 Hz, 1H), 2.82 (d, J=4.6 Hz, 3H), 4.05 (br d, J=18.7 Hz, 1H), 4.37-4.69 (m, 2H), 5.56 (br d, J=2.6 Hz, 1H), 7.28 (d, J=8.4 Hz, 2H), 7.41 (dd, J=8.1, 2.0 Hz, 1H), 7.67 (d, J=2.0 Hz, 1H), 7.69 (d, J=8.1 Hz, 1H), 7.94 (d, J=8.6 Hz, 2H), 8.15-8.25 (m, 1H); LCMS (method C): Rt 0.91 min

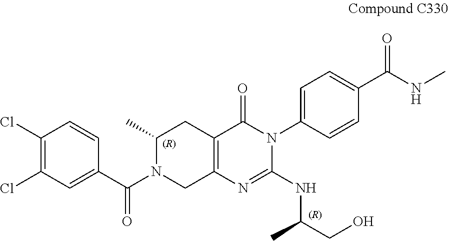

¹H NMR (400 MHz, DMSO-d₆, 81° C.) δ ppm 1.03 (d, J=6.6 Hz, 3H), 1.18 (d, J=6.8 Hz, 3H), 2.33 (d, J=16.5 Hz, 1H), 2.54 (br dd, J=16.5, 5.9 Hz, 1H), 2.83 (d, J=4.6 Hz, 3H), 3.22-3.41 (m, 2H), 3.90-4.09 (m, 2H), 4.42 (t, J=5.4 Hz, 1H), 4.31-4.68 (m, 2H), 4.95 (d, J=7.7 Hz, 1H), 7.36 (br d, J=8.1 Hz, 2H), 7.42 (dd, J=8.4, 2.0 Hz, 1H), 7.68 (d, J=2.0 Hz, 1H), 7.70 (d, J=8.1 Hz, 1H), 7.99 (d, J=8.8 Hz, 2H), 8.28-8.38 (m, 1H); LCMS (method C): Rt 0.84 min

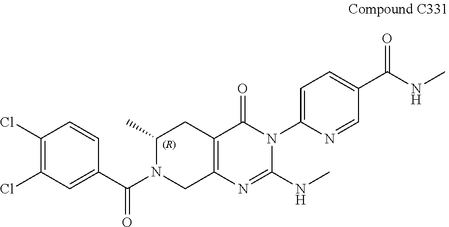

¹H NMR (400 MHz, DMSO-d₆, 81° C.) δ ppm 1.18 (d, J=6.8 Hz, 3H), 2.33 (d, J=16.3 Hz, 1H), 2.54 (dd, J=16.3, 5.7 Hz, 1H), 2.70 (d, J=4.5 Hz, 3H), 2.85 (d, J=4.9 Hz, 3H), 4.04 (br d, J=18.3 Hz, 1H), 4.26-4.87 (m, 2H), 5.91-6.07 (m, 1H), 7.42 (dd, J=8.3, 1.8 Hz, 1H), 7.51 (dd, J=8.1, 0.8 Hz, 1H), 7.68 (d, J=2.0 Hz, 1H), 7.70 (d, J=8.5 Hz, 1H), 8.35 (dd, J=8.3, 2.2 Hz, 1H), 8.44-8.54 (m, 1H), 9.01 (dd, J=2.4, 0.8 Hz, 1H); LCMS (method C): Rt 0.83 min

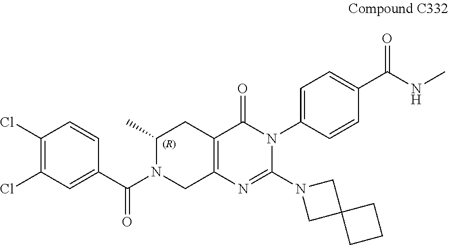

¹H NMR (400 MHz, DMSO-d₆, 81° C.) δ ppm 1.17 (d, J=6.8 Hz, 3H), 1.62 (quin, J=7.6 Hz, 2H), 1.89-1.99 (m, 4H), 2.32 (d, J=16.5 Hz, 1H), 2.50-2.58 (m, 1H), 2.83 (d, J=4.6 Hz, 3H), 3.41-3.51 (m, 4H), 4.02 (br d, J=18.3 Hz, 1H), 4.22-4.78 (m, 2H), 7.34-7.44 (m, 3H), 7.68 (d, J=2.0 Hz, 1H), 7.70 (d, J=8.4 Hz, 1H), 7.93 (d, J=8.8 Hz, 2H), 8.29-8.37 (m, 1H); LCMS (method C): Rt 1.06 min

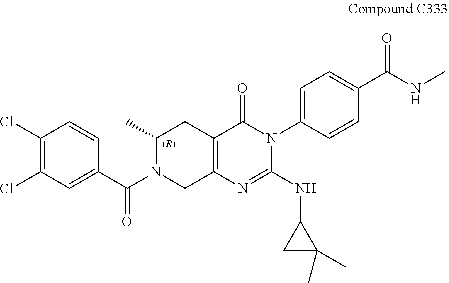

¹H NMR (400 MHz, DMSO-d₆, 81° C.) δ ppm 0.34 (t, J=4.8 Hz, 1H), 0.55 (dd, J=7.7, 5.5 Hz, 1H), 0.88 (s, 3H), 1.03 (s, 3H), 1.18 (d, J=6.6 Hz, 3H), 2.32 (br d, J=16.3 Hz, 1H), 2.35-2.41 (m, 1H), 2.56 (br dd, J=16.4, 5.8 Hz, 1H), 2.82 (d, J=4.4 Hz, 3H), 4.02 (br d, J=18.3 Hz, 1H), 4.21-4.81 (m, 2H), 5.48-5.66 (m, 1H), 7.23-7.38 (m, 2H), 7.42 (dd, J=8.1, 2.0 Hz, 1H), 7.68 (d, J=1.8 Hz, 1H), 7.70 (d, J=8.4 Hz, 1H), 7.91-8.00 (m, 2H), 8.27-8.34 (m, 1H); LCMS (method C): Rt 1.05 min

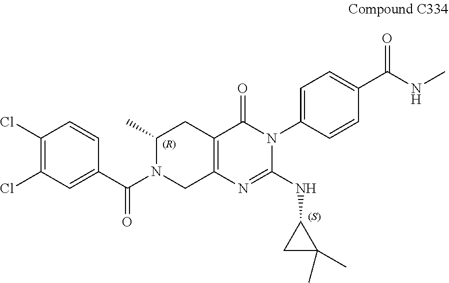

¹H NMR (400 MHz, DMSO-d₆, 81° C.) δ ppm 0.34 (t, J=4.7 Hz, 1H), 0.55 (dd, J=7.7, 5.5 Hz, 1H), 0.88 (s, 3H), 1.03 (s, 3H), 1.18 (d, J=6.6 Hz, 3H), 2.32 (br d, J=16.5 Hz, 1H), 2.35-2.40 (m, 1H), 2.56 (br dd, J=16.5, 6.2 Hz, 1H), 2.82 (d, J=4.4 Hz, 3H), 4.02 (br d, J=18.9 Hz, 1H), 4.22-4.94 (m, 2H), 5.53-5.61 (m, 1H), 7.21-7.39 (m, 2H), 7.42 (dd, J=8.1, 2.0 Hz, 1H), 7.68 (d, J=1.8 Hz, 1H), 7.70 (d, J=8.1 Hz, 1H), 7.96 (d, J=8.8 Hz, 2H), 8.25-8.37 (m, 1H); LCMS (method C): Rt 1.05 min

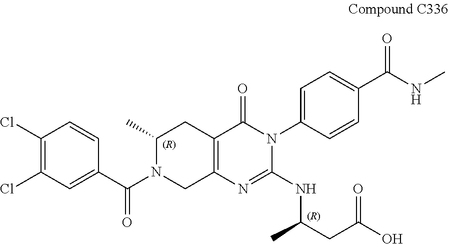

¹H NMR (400 MHz, DMSO-d₆, 81° C.) δ ppm 1.09 (d, J=6.6 Hz, 3H), 1.18 (d, J=6.8 Hz, 3H), 2.33 (br d, J=16.1 Hz, 1H), 2.39 (t, J=5.7 Hz, 2H), 2.51-2.58 (m, 2H), 2.83 (d, J=4.6 Hz, 3H), 3.97-4.08 (m, 1H), 4.24-4.75 (m, 3H), 5.55 (br d, J=8.4 Hz, 1H), 7.32 (d, J=8.6 Hz, 2H), 7.42 (dd, J=8.1, 2.0 Hz, 1H), 7.68 (d, J=1.8 Hz, 1H), 7.70 (d, J=8.4 Hz, 1H), 7.98 (d, J=8.4 Hz, 2H), 8.30-8.37 (m, 1H); LCMS (method D): Rt 1.40 min

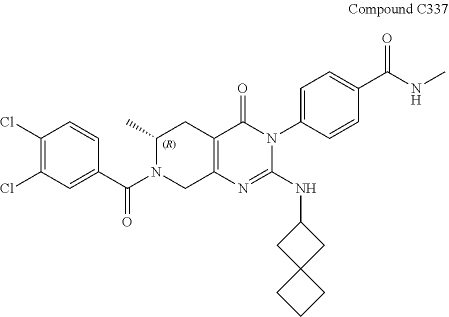

¹H NMR (400 MHz, DMSO-d₆, 81° C.) δ ppm 1.17 (d, J=6.9 Hz, 3H), 1.67-1.92 (m, 6H), 1.98 (t, J=7.1 Hz, 2H), 2.18-2.28 (m, 2H), 2.31 (br d, J=16.3 Hz, 1H), 2.50-2.58 (m, 1H), 2.83 (d, J=4.5 Hz, 3H), 4.00 (br d, J=18.7 Hz, 1H), 4.19 (sxt, J=7.7 Hz, 1H), 4.28-4.74 (m, 2H), 5.47 (br d, J=6.9 Hz, 1H), 7.31 (br d, J=8.1 Hz, 2H), 7.41 (dd, J=8.1, 2.0 Hz, 1H), 7.66 (d, J=1.6 Hz, 1H), 7.70 (d, J=8.1 Hz, 1H), 7.97 (d, J=9.0 Hz, 2H), 8.23-8.32 (m, 1H); LCMS (method C): Rt 1.17 min

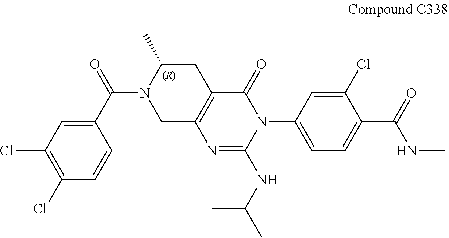

¹H NMR (400 MHz, DMSO-d₆, 100° C.) δ ppm 1.08 (d, J=6.5 Hz, 3H), 1.07 (d, J=6.5 Hz, 3H), 1.18 (d, J=6.8 Hz, 3H), 2.32 (d, J=16.0 Hz, 1H), 2.50-2.57 (m, 1H), 2.75-2.84 (m, 3H), 4.01 (br d, J=18.9 Hz, 1H), 4.15 (dq, J=13.8, 6.7 Hz, 1H), 4.37-4.63 (m, 2H), 5.26 (br d, J=7.7 Hz, 1H), 7.26 (dd, J=8.0, 1.9 Hz, 1H), 7.36-7.45 (m, 2H), 7.57 (d, J=7.9 Hz, 1H), 7.65 (d, J=2.0 Hz, 1H), 7.69 (d, J=8.1 Hz, 1H), 8.04-8.13 (m, 1H); LCMS (method D): Rt 1.95 min

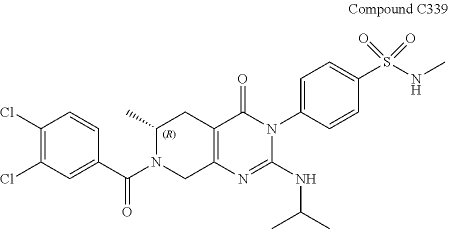

¹H NMR (400 MHz, CDCl₃) δ ppm 8.04-8.11 (m, 2H), 7.51-7.58 (m, 2H), 7.44-748 (m, 1H), 7.39-7.43 (m, 1H), 7.27-7.30 (m, 1H), 4.85-5.44 (m, 1H), 4.58 (q, J=5.3 Hz, 1H), 3.91-4.41 (m, 3H), 3.66-3.76 (m, 1H), 2.75 (d, J=5.3 Hz, 3H), 2.62-2.73 (m, 1H), 2.51 (br d, J=16.3 Hz, 1H), 1.26 (br d, J=4.1 Hz, 3H), 1.11 (br d, J=6.1 Hz, 6H); LCMS (method C): Rt 1.07 min

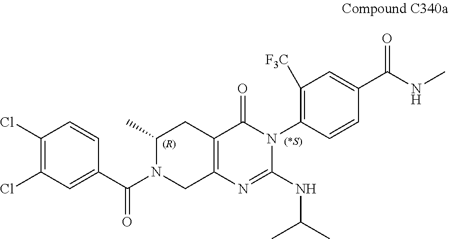

¹H NMR (400 MHz, DMSO-d₆, 81° C.) δ ppm 0.99-1.06 (m, 6H), 1.15 (d, J=6.8 Hz, 3H), 2.30 (d, J=16.3 Hz, 1H), 2.50-2.56 (m, 1H), 2.86 (d, J=4.6 Hz, 3H), 4.03 (br d, J=19.4 Hz, 1H), 4.21 (dq, J=13.7, 6.9 Hz, 1H), 4.30-4.76 (m, 2H), 5.54 (d, J=7.9 Hz, 1H), 7.41 (dd, J=8.1, 2.0 Hz, 1H), 7.53 (d, J=8.4 Hz, 1H), 7.68 (d, J=2.0 Hz, 1H), 7.70 (d, J=8.1 Hz, 1H), 8.25 (dd, J=8.1, 1.8 Hz, 1H), 8.31 (d, J=1.8 Hz, 1H), 8.58-8.65 (m, 1H); LCMS (method D): Rt 2.10 min

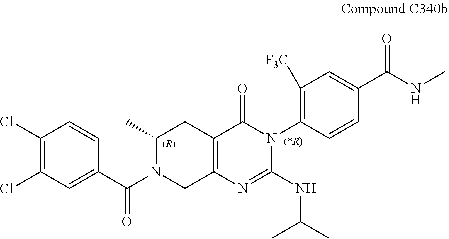

¹H NMR (400 MHz, DMSO-d₆, 81° C.) δ ppm 1.01 (d, J=6.4 Hz, 3H), 1.05 (d, J=6.6 Hz, 3H), 1.17 (d, J=6.8 Hz, 3H), 2.31 (br d, J=16.5 Hz, 1H), 2.50-2.57 (m, 1H), 2.86 (d, J=4.6 Hz, 3H), 4.00 (br d, J=18.5 Hz, 1H), 4.15-4.25 (m, 1H), 4.32-4.73 (m, 2H), 5.52 (d, J=7.9 Hz, 1H), 7.44 (dd, J=8.1, 2.0 Hz, 1H), 7.56 (d, J=8.1 Hz, 1H), 7.68-7.71 (m, 2H), 8.24 (dd, J=8.1, 1.8 Hz, 1H), 8.31 (d, J=1.8 Hz, 1H), 8.58-8.64 (m, 1H); LCMS (method D): Rt 2.07 min

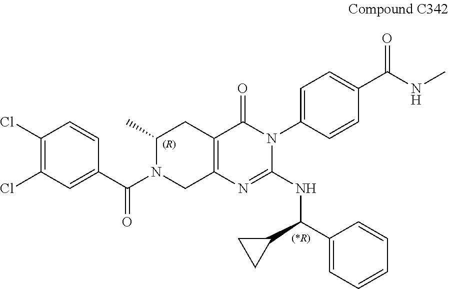

¹H NMR (400 MHz, DMSO-d₆, 81° C.) δ ppm 0.28-0.38 (m, 2H), 0.39-0.53 (m, 2H), 1.12 (d, J=6.8 Hz, 3H), 1.17-1.24 (m, 1H), 2.30 (br d, J=16.1 Hz, 1H), 2.51-2.57 (m, 1H), 2.83 (d, J=4.6 Hz, 3H), 3.88 (br d, J=18.3 Hz, 1H), 4.23-4.65 (m, 2H), 4.45 (t, J=8.5 Hz, 1H), 5.80 (d, J=7.9 Hz, 1H), 7.16-7.22 (m, 1H), 7.24-7.36 (m, 4H), 7.37-7.42 (m, 3H), 7.66 (d, J=2.0 Hz, 1H), 7.70 (d, J=8.4 Hz, 1H), 8.01 (d, J=8.6 Hz, 2H), 8.31-8.38 (m, 1H); LCMS (method D): Rt 2.20 min

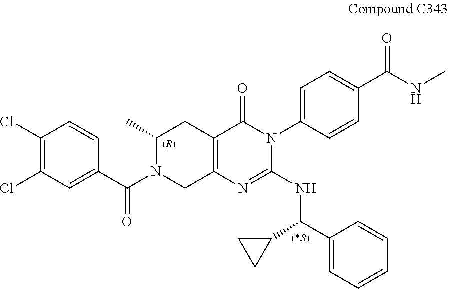

¹H NMR (400 MHz, DMSO-d₆, 81° C.) δ ppm 0.24-0.38 (m, 2H), 0.40-0.52 (m, 2H), 1.17 (d, J=6.8 Hz, 3H), 1.10-1.29 (m, 1H), 2.30 (br d, J=16.3 Hz, 1H), 2.50-2.56 (m, 1H), 2.83 (d, J=4.4 Hz, 3H), 3.93 (br d, J=19.4 Hz, 1H), 4.19-4.66 (m, 2H), 4.39 (br t, J=8.4 Hz, 1H), 5.79 (br d, J=7.7 Hz, 1H), 7.14-7.47 (m, 8H), 7.63 (d, J=1.5 Hz, 1H), 7.67 (d, J=8.4 Hz, 1H), 8.02 (d, J=8.8 Hz, 2H), 8.29-8.40 (m, 1H); LCMS (method D): Rt 2.16 min

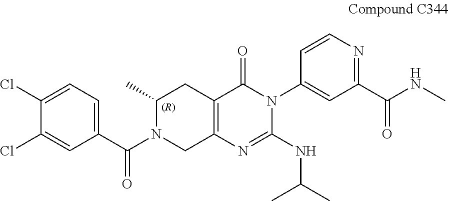

¹H NMR (400 MHz, DMSO-d₆, 100° C.) δ ppm 1.04-1.08 (m, 6H), 1.19 (d, J=6.8 Hz, 3H), 2.32 (d, J=16.5 Hz, 1H), 2.53-2.58 (m, 1H), 2.88 (d, J=5.1 Hz, 3H), 4.01 (br d, J=18.5 Hz, 1H), 4.16 (dq, J=13.7, 6.7 Hz, 1H), 4.39-4.62 (m, 2H), 5.52 (br d, J=7.5 Hz, 1H), 7.41 (dd, J=8.3, 1.9 Hz, 1H), 7.50 (dd, J=5.2, 2.1 Hz, 1H), 7.66 (d, J=2.0 Hz, 1H), 7.69 (d, J=8.1 Hz, 1H), 7.86 (d, J=1.5 Hz, 1H), 8.50-8.57 (m, 1H), 8.77 (dd, J=5.1, 0.7 Hz, 1H); LCMS (method C): Rt 1.01 min

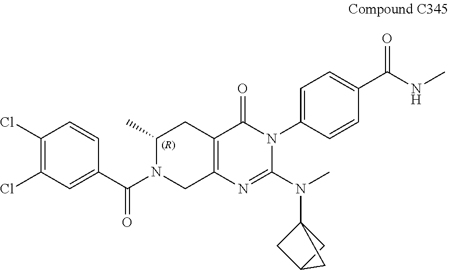

¹H NMR (400 MHz, DMSO-d₆, 80° C.) δ ppm 1.17 (d, J=6.6 Hz, 3H), 1.22-1.32 (m, 4H), 1.90-1.97 (m, 6H), 2.36 (d, J=16.9 Hz, 1H), 2.58 (br dd, J=16.9, 5.9 Hz, 1H), 2.81 (d, J=4.6 Hz, 3H), 4.07 (b d, J=17.6 Hz, 1H), 4.27-4.80 (m, 2H), 7.35-7.40 (m, 2H), 7.42 (dd, J=8.1, 2.0 Hz, 1H), 7.68 (d, J=1.8 Hz, 1H), 7.70 (d, J=8.1 Hz, 1H), 7.90 (d, J=8.6 Hz, 2H), 8.24-8.33 (m, 1H); LCMS (method C): Rt 1.10 min

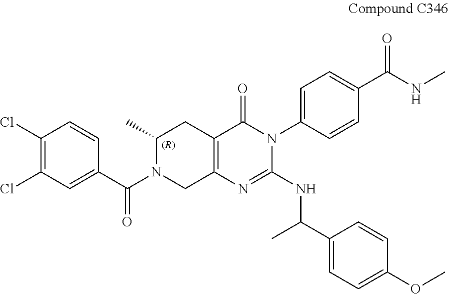

¹H NMR (400 MHz, DMSO-d₆, 81° C.) δ ppm 1.16 (dd, J=15.0, 6.8 Hz, 3H), 1.34 (d, J=7.0 Hz, 3H), 2.31 (br d, J=16.3 Hz, 1H), 2.51-2.57 (m, 1H), 2.83 (d, J=4.6 Hz, 3H), 3.71 (s, 3H), 3.90-4.07 (m, 1H), 4.23-4.74 (m, 2H), 5.11-5.23 (m, 1H), 5.57 (dd, J=12.0, 8.0 Hz, 1H), 6.78-6.85 (m, 2H), 7.13-7.20 (m, 2H), 7.31-7.43 (m, 3H), 7.64-7.72 (m, 2H), 7.99 (d, J=8.6 Hz, 2H), 8.29-8.36 (m, 1H); LCMS (method C): Rt 1.08 min

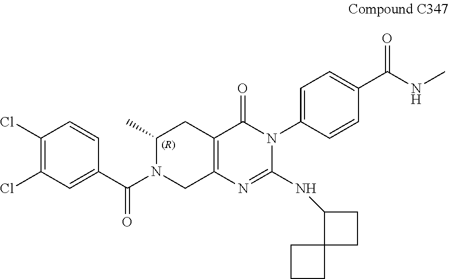

¹H NMR (400 MHz, DMSO-d₆, 81° C.) δ ppm 1.18 (d, J=6.8 Hz, 3H), 1.52-1.97 (m, 8H), 2.12-2.24 (m, 1H), 2.29-2.38 (m, 1H), 2.52-2.61 (m, 1H), 2.83 (d, J=4.6 Hz, 3H), 3.94-4.07 (m, 1H), 4.16-4.28 (m, 1H), 4.31-4.81 (m, 2H), 5.21 (dd, J=16.0, 7.2 Hz, 1H), 7.37-7.44 (m, 3H), 7.67-7.72 (m, 2H), 8.00 (d, J=8.4 Hz, 2H), 8.30-8.37 (m, 1H); LCMS (method C): Rt 1.15 min

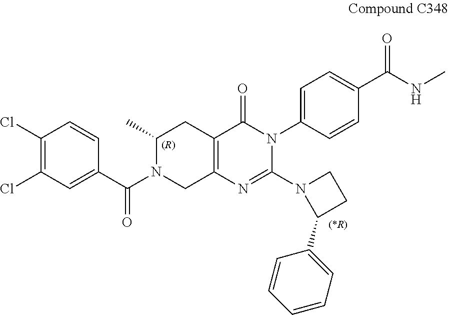

¹H NMR (400 MHz, DMSO-d₆, 81° C.) δ ppm 1.18 (d, J=6.8 Hz, 3H), 1.80-1.93 (m, 1H), 2.32 (d, J=16.7 Hz, 1H), 2.40-2.46 (m, 1H), 2.50-2.56 (m, 1H), 2.72 (td, J=8.8 Hz, 1H), 2.82 (d, J=4.4 Hz, 3H), 3.64 (q, J=7.9 Hz, 1H), 3.96 (br d, J=18.5 Hz, 1H), 4.10-4.65 (m, 2H), 5.34 (br t, J=7.9 Hz, 1H), 7.20-7.38 (m, 6H), 7.39-7.45 (m, 2H), 7.62 (d, J=2.0 Hz, 1H), 7.66 (d, J=8.1 Hz, 1H), 7.86-8.00 (m, 2H), 8.27-8.35 (m, 1H); LCMS (method D): Rt 2.08 min

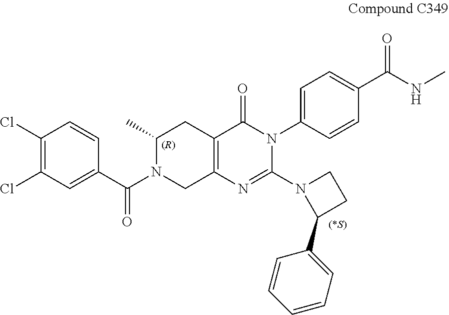

¹H NMR (400 MHz, DMSO-d₆, 81° C.) δ ppm 1.10 (d, J=6.8 Hz, 3H), 1.78-1.89 (m, 1H), 2.32 (d, J=16.7 Hz, 1H), 2.42-2.47 (m, 1H), 2.56 (br dd, J=16.7, 6.2 Hz, 1H), 2.77 (td, J=8.7, 5.1 Hz, 1H), 2.82 (d, J=4.6 Hz, 3H), 3.61 (q, J=8.1 Hz, 1H), 3.85 (br d, J=18.9 Hz, 1H), 4.25-4.72 (m, 2H), 5.31 (dd, J=8.8, 7.3 Hz, 1H), 7.21-7.36 (m, 5H), 7.36-7.45 (m, 3H), 7.66 (d, J=1.8 Hz, 1H), 7.70 (d, J=8.1 Hz, 1H), 7.83-8.02 (m, 2H), 8.24-8.35 (m, 1H); LCMS (method D): Rt 2.09 min

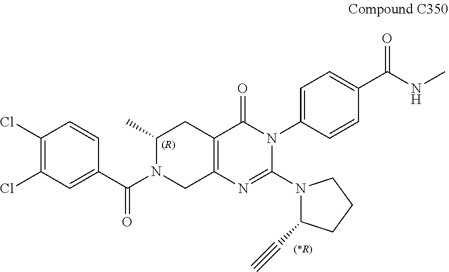

¹H NMR (400 MHz, DMSO-d₆, 81° C.) δ ppm 1.17 (d, J=6.8 Hz, 3H), 1.53-1.79 (m, 3H), 1.97-2.07 (m, 1H), 2.36 (d, J=16.7 Hz, 1H), 2.59 (br dd, J=16.6, 6.1 Hz, 1H), 2.59-2.72 (m, 1H), 2.81 (d, J=4.6 Hz, 3H), 2.95-3.03 (m, 2H), 4.06 (br d, J=19.2 Hz, 1H), 4.30 (ddd, J=7.2, 5.6, 2.0 Hz, 1H), 4.37-4.78 (m, 2H), 7.30-7.48 (m, 3H), 7.67-7.73 (m, 2H), 7.89-7.94 (m, 2H), 8.24-8.36 (m, 1H); LCMS (method D): Rt 1.97 min

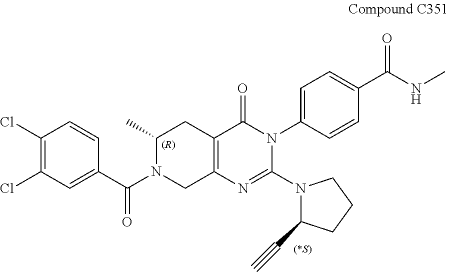

¹H NMR (400 MHz, DMSO-d₆, 81° C.) δ ppm 1.19 (d, J=6.8 Hz, 3H), 1.55-1.78 (m, 3H), 2.02-2.11 (m, 1H), 2.35 (d, J=16.5 Hz, 1H), 2.51-2.60 (m, 2H), 2.81 (d, J=4.6 Hz, 3H), 3.00-3.04 (m, 2H), 4.06 (br d, J=19.4 Hz, 1H), 4.28-4.81 (m, 2H), 4.39 (br t, J=5.8 Hz, 1H), 7.24-7.51 (m, 3H), 7.67-7.72 (m, 2H), 7.89-7.95 (m, 2H), 8.26-8.35 (m, 1H); LCMS (method D): Rt 1.97 min

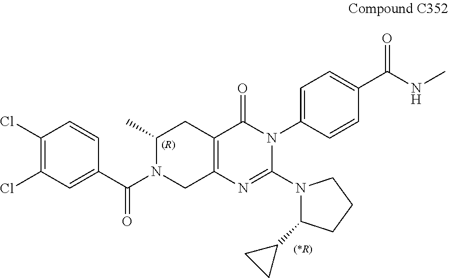

¹H NMR (400 MHz, DMSO-d₆, 81° C.) δ ppm 0.09-0.17 (m, 1H), 0.31-0.47 (m, 3H), 0.91-1.01 (m, 1H), 1.17 (d, J=6.8 Hz, 3H), 1.27-1.38 (m, 1H), 1.46-1.66 (m, 2H), 1.75-1.85 (m, 1H), 2.20 (td, J=9.5, 6.9 Hz, 1H), 2.36 (d, J=16.5 Hz, 1H), 2.58 (br dd, J=16.5, 5.9 Hz, 1H), 2.81 (d, J=4.6 Hz, 3H), 2.91-2.98 (m, 1H), 3.95-4.09 (m, 2H), 4.35-4.70 (m, 2H), 7.31-7.45 (m, 2H), 7.42 (dd, J=8.4, 2.0 Hz, 1H), 7.68 (d, J=2.0 Hz, 1H), 7.71 (d, J=8.1 Hz, 1H), 7.89-7.94 (m, 2H), 8.25-8.33 (m, 1H); LCMS (method D): Rt 2.12 min

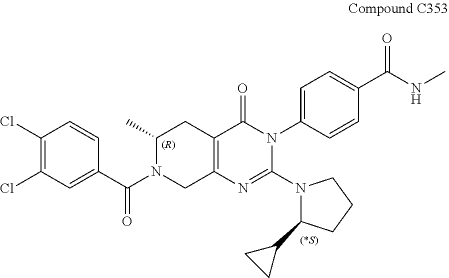

¹H NMR (400 MHz, DMSO-d₆, 81° C.) δ ppm 0.08-0.17 (m, 1H), 0.30-0.49 (m, 3H), 0.92-1.02 (m, 1H), 1.18 (d, J=6.6 Hz, 3H), 1.28-1.38 (m, 1H), 1.47-1.66 (m, 2H), 1.78-1.88 (m, 1H), 2.15 (td, J=9.7, 6.8 Hz, 1H), 2.34 (d, J=16.7 Hz, 1H), 2.55 (br dd, J=16.6, 6.0 Hz, 1H), 2.81 (d, J=4.6 Hz, 3H), 2.95-3.02 (m, 1H), 3.97-4.08 (m, 2H), 4.24-4.77 (m, 2H), 7.25-7.49 (m, 3H), 7.67-7.72 (m, 2H), 7.88-7.94 (m, 2H), 8.26-8.33 (m, 1H); LCMS (method D): Rt 2.12 min

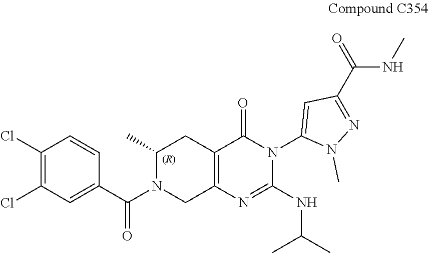

¹H NMR (400 MHz, CDCl₃) δ ppm 1.07-1.20 (m, 6H), 1.25 (d, J=5.7 Hz, 3H), 2.45-2.55 (m, 1H), 2.67 (br d, J=13.4 Hz, 1H), 3.00 (d, J=4.9 Hz, 3H), 3.69 (d, J=6.1 Hz, 3H), 3.93-3.46 (m, 4H), 4.76-5.43 (m, 1H), 6.81-6.89 (m, 2H), 7.24-7.30 (m, 1H) 7.50-7.55 (m, 2H); LCMS (method C): Rt 0.98 min

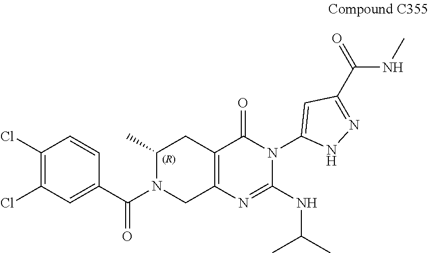

¹H NMR (400 MHz, DMSO-d₆) δ ppm 0.95-1.22 (m, 10H), 2.18-2.41 (m, 1H), 2.44-2.56 (m, 1H), 2.80 (d, J=4.4 Hz, 3H), 3.76-4.31 (m, 3H), 4.74-5.19 (m, 1H), 5.88-5.99 (m, 1H), 6.71-6.78 (9, 1H), 7.39-7.54 (m, 1H), 7.74 (d, J=8.4 Hz, 2H), 8.41-8.53 (m, 1H); LCMS (method C): Rt 0.95 min

SFC data

| Cmpd no | Rt (min) | [M + H]⁺ | [M − H]⁻ | SFC method |
|---|---|---|---|---|
| C375a | 5.98 | 591 | 589 | SFC_A |
| C375b | 5.21 | 591 | 589 | SFC_A |
| C372b | 3.66 | 594 | 592 | SFC_E |
| C372a | 3.9 | 591 | 592 | SFC_E |
| C377b | 3.65 | 591 | 589 | SFC_E |
| C377a | 3.83 | 591 | 589 | SFC_E |
| C383a | 6.81 | 556 | 554 | SFC_D |
| C383b | 8.14 | 556 | 554 | SFC_D |
| C386a | 7.08 | 624 | 622 | SFC_D |
| C386b | 7.78 | 624 | 622 | SFC_D |
| C387a | 6.97 | 588 | 586 | SFC_D |
| C387b | 7.72 | 588 | 586 | SFC_D |
| C392a | 2.84 | 585 | 583 | SFC_F |
| C392b | 3.08 | 585 | 583 | SFC_F |
| C346b | 4.41 | 580 | 578 | SFC_E |
| C394a | 5.16 | 620 | 618 | SFC_A |
| C346a | 4.24 | 580 | 578 | SFC_E |
| C394b | 5.64 | 620 | 618 | SFC_A |
| C347a | 5.39 | 620 | 618 | SFC_A |
| C347b | 5.77 | 620 | 618 | SFC_A |
| C401b | 7.83 | 616 | 614 | SFC_D |
| C401a | 7.21 | 616 | 614 | SFC_D |
| C402a | 4.12 | 620 | 618 | SFC_E |
| C402b | 4.78 | 620 | 618 | SFC_E |
| C403a | 6.9 | 622 | 561 | SFC_D |
| C403b | 8.56 | 622 | 561 | SFC_D |
| C393a | 5.81 | 571 | 569 | SFC_D |
| C393b | 6.38 | 571 | 569 | SFC_D |
| C399a | 6.61 | 551 | 549 | SFC_I |
| C399b | 7.42 | 551 | 549 | SFC_I |
| C406b | 5.11 | 568 | 566 | SFC_A |
| C406a | 5.33 | 568 | 566 | SFC_A |
| C411a | 7.75 | 656 | 654 | SFC_D |
| C414b | 7.17 | 634 | 632 | SFC_L |
| C415a | 4.66 | 650 | 648 | SFC_E |
| C416b | 5.25 | 638 | 636 | SFC_A |
| C415b | 5.17 | 650 | 648 | SFC_E |
| C416a | 5.58 | 638 | 636 | SFC_A |
| C411b | 7.08 | | | SFC_M |
| C417b | 7.3 | 626 | 624 | SFC_D |
| C417a | 8.28 | 626 | 624 | SFC_D |
| C414a | 7.5 | 634 | 632 | SFC_L |
| C419 | 2.15 | 532 | 530 | SFC_E |
| C422b | 4.93 | 648 | 646 | SFC_A |
| C422a | 6 | 648 | 646 | SFC_A |
| C412a | 5.61 | 727 | 666 | SFC_J |
| C412b | 6.39 | 727 | 666 | SFC_J |
| C413a | 6.43 | 658 | 656 | SFC_D |
| C413b | 7.14 | 658 | 656 | SFC_D |
| C420a | 2.25 | 570 | 568 | SFC_E |
| C420b | 2.54 | 570 | 568 | SFC_E |
| C431 | 5.38 | 604 | 602 | SFC_A |
| C432 | 2.94 | 598 | 596 | SFC_E |
| C435b | 4.33 | 638 | 636 | SFC_E |
| C435a | 4.84 | 638 | 636 | SFC_E |
| C436b | 4.13 | 634 | 632 | SFC_G |
| C436a | 4.42 | 634 | 632 | SFC_G |
| C439b | 4.89 | 581 | 579 | SFC_B |
| C439a | 5.57 | 581 | 579 | SFC_B |
| C434b | 3.97 | 597 | 595 | SFC_G |
| C434a | 4.72 | 597 | 595 | SFC_G |
| C440a | 6.81 | 674 | 672 | SFC_D |
| C441a | 5.28 | 615 | 613 | SFC_J |
| C441a | 5.65 | 615 | 613 | SFC_J |
| C433b | 4.96 | 595 | 593 | SFC_A |
| C433a | 5.77 | 595 | 593 | SFC_A |
| C443 | 7.16 | 658 | 656 | SFC_K |
| C445 | 4.17 | 624 | 622 | SFC_G |
| C449a | 6.17 | 629 | 627 | SFC_B |
| C449b | 6.62 | 629 | 627 | SFC_B |
| C450a | 5.55 | 643 | 641 | SFC_H |
| C450b | 6.28 | 643 | 641 | SFC_H |
| C437b | 5.07 | 630 | 628 | SFC_A |
| C437a | 5.69 | 630 | 628 | SFC_A |
| C438b | 7.05 | 656 | 654 | SFC_A |
| C438a | 7.79 | 656 | 654 | SFC_A |
| C447a | 2.67 | 555 | 553 | SFC_E |
| C447b | 3.58 | 555 | 553 | SFC_E |
| C455a | 6.12 | 585 | 583 | SFC_D |
| C455b | 6.89 | 585 | 583 | SFC_D |
| C452a | 5.62 | 632 | 630 | SFC_A |
| C452b | 6.05 | 632 | 630 | SFC_A |
| C410b | 4.8 | 620 | 618 | SFC_N |
| C410a | 5.59 | 620 | 618 | SFC_N |
| C462a | 5.36 | 630 | 628 | SFC_E |
| C462b | 5.77 | 630 | 628 | SFC_E |
| C459a | 8.1 | 570 | 568 | SFC_I |
| C459b | 8.46 | 570 | 568 | SFC_I |
| C463a | 5.31 | 683 | 681 | SFC_B |
| C526a | 6.48 | 618 | 616 | SFC_A |
| C526b | 7.84 | 618 | 616 | SFC_A |
| C527a | 5.43 | 648 | 646 | SFC_O |
| C527b | 5.7 | 648 | 646 | SFC_O |
| C528a | 4.38 | 647 | 645 | SFC_G |
| C528b | 4.75 | 647 | 645 | SFC_G |
| C529a | 6.64 | 630 | 628 | SFC_J |
| C529b | 7.33 | 630 | 628 | SFC_J |
| C463b | 5.76 | 683 | 681 | SFC_B |
| C530a | 6.03 | 647 | 645 | SFC_A |
| C530b | 7.08 | 647 | 645 | SFC_A |
| C535a | 6.63 | 644 | 642 | SFC_K |
| C535b | 7.08 | 644 | 642 | SFC_K |
| C607 | 5.61 | 664 | 662 | SFC_D |
| C608b | 4.68 | 672 | 670 | SFC_J |
| C609 | 4.34 | 640 | 638 | SFC_E |
| C608a | 3.15 | 672 | 670 | SFC_E |
| C614 | 4.24 | 610 | 608 | SFC_E |
| C615 | 3.8 | 660 | 658 | SFC_E |
| C617 | 3.24 | 676 | 674 | SFC_G |
| C621 | 5.34 | 622 | 620 | SFC_A |
| C622 | 2.68 | 678 | 676 | SFC_E |
| C623 | 2.65 | 662 | 660 | SFC_E |
| C624 | 3.11 | 612 | 610 | SFC_E |
| C630 | 2.92 | 648 | 646 | SFC_E |
| C639a | 5.01 | 596 | 594 | SFC_D |
| C639b | 5.57 | 596 | 594 | SFC_D |
| C643a | 2.67 | 592 | 590 | SFC_P |
| C647a | 5.5 | 630 | 628 | SFC_A |
| C647b | 6.35 | 630 | 628 | SFC_A |
| C643b | 3.08 | 592 | 590 | SFC_Q |
| C650 | 3.18 | 692 | 690 | SFC_A |
| C652a | 4.37 | 692 | 690 | SFC_B |
| C652b | 4.78 | 692 | 690 | SFC_B |
| C658 | 5.63 | 646 | 644 | SFC_D |
| C659a | 6.16 | 676 | 674 | SFC_D |
| C659b | 6.93 | 676 | 674 | SFC_D |
| C660a | 3.62 | 640 | 638 | SFC_E |
| C660b | 4.02 | 640 | 638 | SFC_E |
| C661a | 5.15 | 602 | 600 | SFC_D |
| C661b | 6.64 | 602 | 600 | SFC_D |
| C663a | 3.84 | 676 | 674 | SFC_A |
| C663b | 4.3 | 676 | 674 | SFC_A |
| C664a | 6.51 | 692 | 690 | SFC_D |
| C664b | 7.3 | 692 | 690 | SFC_D |
| C665a | 5.95 | 626 | 624 | SFC_C |
| C665b | 6.48 | 626 | 624 | SFC_C |
| C669a | 5.22 | 612 | 610 | SFC_K |
| C669b | 5.8 | 612 | 610 | SFC_K |
| C676a | 5.08 | 622 | 620 | SFC_D |
| C676b | 5.54 | 622 | 620 | SFC_D |
| C677b | 5.5 | 610 | 608 | SFC_D |
| C685a | 6.21 | 578 | 576 | SFC_R |
| C685b | 6.89 | 578 | 576 | SFC_R |
| C678a | 2.75 | 618 | 616 | SFC_Q |
| C678b | 3.26 | 618 | 616 | SFC_Q |
| C689a | 4.67 | 626 | 624 | SFC_J |
| C689b | 5.26 | 626 | 624 | SFC_J |
| C692 | 5.68 | 552 | 550 | SFC_A |
| C693 | 5.36 | 532 | 530 | SFC_A |
| C697a | 5.63 | 643 | 641 | SFC_E |
| C697b | 6.8 | 643 | 641 | SFC_E |
| C677a | 4.82 | 610 | 608 | SFC_D |
| C699 | 4.01 | 570 | 568 | SFC_A |
| C701a | 6.55 | 620 | 618 | SFC_D |
| C701b | 7.26 | 620 | 618 | SFC_D |

LCMS data for compounds

| | Rt (min) | [M + H]+ | [M − H]− | LCMS method |
|---|---|---|---|---|
| C356 | 1.46 | 507 | 505 | K |
| C357 | 1.97 | 540 | 538 | K |
| C358 | 1.71 | 507 | 505 | K |
| C359 | 1.44 | 507 | 505 | K |
| C360 | 1.87 | 509 | 507 | A |
| C361 | 1.46 | 507 | 505 | A |
| C362 | 1.84 | 508 | 506 | K |
| C363 | 1.43 | 521 | 519 | K |
| C364 | 1.56 | 508 | 506 | K |
| C365 | 1.43 | 508 | 506 | A |
| C366 | 1.63 | 524 | 522 | K |
| C367 | 1.77 | 536 | 534 | K |
| C368 | 1.47 | 507 | 505 | K |
| C369 | 1.40 | 507 | 505 | D |
| C370 | 2.26 | 535 | 533 | A |
| C371 | 1.18 | 553 | 551 | C |
| C372 | 0.91 | 594 | 592 | C |
| C373 | 1.11 | 566 | 564 | C |
| C374 | 0.77 | 521 | 519 | C |
| C375a | 1.81 | 591 | 589 | D |
| C375b | 1.82 | 591 | 589 | D |
| C376 | 1.13 | 596 | 594 | C |
| C372b | 1.78 | 594 | 592 | D |
| C372a | 1.79 | 594 | 592 | D |
| C377b | 1.79 | 591 | 589 | D |
| C378 | 1.73 | 577 | 575 | D |
| C377a | 1.80 | 591 | 589 | D |
| C379 | 1.09 | 535 | 533 | C |
| C380 | 1.10 | 608 | 606 | C |
| I70 | 1.63 | 515 | 513 | K |
| C381 | 1.05 | 554 | 552 | C |
| C382 | 2.04 | 558 | 556 | D |
| C383a | 2.11 | 556 | 554 | D |
| C383b | 2.08 | 556 | 554 | D |
| C384 | 1.83 | 395 | 393 | D |
| C385 | 1.14 | 599 | 597 | C |
| C386a | 2.10 | 624 | 622 | K |
| C386b | 2.16 | 624 | 622 | K |
| C386 | 2.13-2.19 | 624 | 621 | K |
| C387a | 2.14 | 588 | 586 | D |
| C387b | 2.17 | 588 | 586 | D |
| C387 | 2.10-2.13 | 588 | 586 | K |
| C388 | 1.14 | 592 | 592 | C |
| C389 | 0.81 | 566 | 985 | C |
| C390 | 1.11 | 556 | 554 | C |
| C391 | 2.04 | 556 | 554 | D |
| C392a | 2.11 | 585 | 583 | D |
| C392b | 2.09 | 585 | 583 | D |
| C393 | 1.06 | 571 | 569 | C |
| C346b | 2.20 | 580 | 578 | D |
| C394a | 2.09 | 620 | 618 | D |
| C395 | 2.17 | 526 | 524 | K |
| C396 | 2.14 | 568 | 566 | D |
| C346a | 2.25 | 580 | 578 | D |
| C394b | 2.08 | 620 | 618 | D |
| C397 | 10.5 | 543 | | J |
| C398 | 10.5 | 543 | | J |
| C399 | 1.96 | 549 | 551 | D |
| C347a | 2.06 | 620 | 618 | D |
| C347b | 2.06 | 620 | 618 | D |
| C401b | 2.15 | 616 | 614 | D |
| C401a | 2.14 | 616 | 614 | D |
| C402a | 2.13 | 620 | 618 | D |
| C402b | 2.12 | 620 | 618 | D |
| C403a | 2.08 | 563 | 561 | D |
| C403b | 2.09 | 563 | 561 | D |
| C393a | 2.01 | 571 | 569 | D |
| C393b | 2.03 | 571 | 569 | D |
| C404 | 1.24 | 569 | | C |
| C405 | 0.81 | 580 | 580 | C |
| C406b | 2.08 | 568 | 566 | A |
| C406a | 2.18 | 568 | 566 | D |
| C407 | 1.15 | 604 | 602 | C |
| C408 | 1.02 | 552 | 552 | C |
| C409 | 1.04 | 528 | 528 | C |
| C410 | 1.12 | 620 | 618 | C |
| C411a | 2.04 | 656 | 654 | A |
| C412 | 0.97 | 668 | 666 | C |
| C413 | 1.19-1.20 | 658 | 656 | C |
| C414b | 2.02 | 634 | 632 | D |
| C415a | 2.02 | 650 | 648 | D |
| C416b | 2.09 | 638 | 636 | D |
| C415b | 1.97 | 650 | 648 | D |
| C416a | 2.05 | 638 | 636 | D |
| C411b | 1.14 | 656 | 654 | C |
| C417b | 2.10 | 626 | 624 | D |
| C417a | 2.12 | 626 | 624 | D |
| C414a | 2.03 | 634 | 632 | D |
| C418 | 1.84 | 544 | 542 | D |
| C419 | 1.04 | 532 | 530 | C |
| C420 | 2.11-2.22 | 570 | 568 | K |
| C421 | 1.61 | 567 | 565 | A |
| C422b | 2.19 | 648 | 646 | D |
| C422a | 2.20 | 648 | 646 | D |
| C423 | 1.55 | 567 | 565 | D |
| C424 | 1.10 | 560 | 558 | C |
| C412a | 1.83 | 668 | 666 | D |
| C412b | 1.83 | 668 | 666 | D |
| C413a | 2.19 | 658 | 656 | D |
| C413b | 2.21 | 658 | 656 | D |
| C425 | 1.99 | 538 | 536 | A |
| C420a | 2.12 | 570 | 568 | D |
| C420b | 2.16 | 570 | 568 | D |
| C427 | 1.84 | 544 | 542 | K |
| C428 | 2.01 | 562 | 560 | K |
| C429 | 1.89 | 508 | 506 | K |
| C430 | 1.97 | 538 | 536 | A |
| C431 | 1.15 | 604 | 602 | C |
| C433 | 0.98 | 595 | 593 | C |
| C434 | 0.96 | 597 | 595 | C |
| C435b | 2.08 | 638 | 636 | D |
| C435a | 2.07 | 638 | 636 | D |
| C436b | 2.10 | 634 | 632 | D |
| C436a | 2.10 | 634 | 632 | D |
| C437 | 1.23 | 630 | 628 | C |
| C438 | 1.15 | 656 | 654 | C |
| C439b | 1.59 | 581 | 579 | D |
| C439a | 1.59 | 581 | 579 | D |
| C434b | 1.86 | 597 | 595 | D |
| C434a | 1.86 | 597 | 595 | D |
| C440b | 1.23 | 674 | 672 | C |
| C440a | 2.26 | 674 | 672 | D |
| C441a | 1.96 | 615 | 613 | D |
| C441b | 1.97 | 615 | 613 | D |
| C442 | 2.05 | 538 | 536 | D |
| C433b | 1.88 | 595 | 593 | D |
| C433a | 1.88 | 595 | 593 | D |
| C443 | 1.21 | 658 | 656 | C |
| C444 | 2.07 | 566 | 564 | D |
| C445 | 1.19 | 624 | 622 | C |
| C446 | 10.9 | 557 | | J |
| C447 | 2.04 | 555 | 553 | A |
| C448 | 1.07 | 538 | 536 | C |
| C449a | 1.98 | 629 | 627 | D |
| C449b | 1.98 | 629 | 627 | D |
| C450a | 2.16 | 643 | 641 | D |
| C450b | 2.12 | 643 | 641 | D |
| C437b | 2.23 | 630 | 628 | D |
| C437a | 2.23 | 630 | 628 | D |
| C438b | 2.14 | 656 | 654 | D |
| C438a | 2.10 | 656 | 654 | D |
| C451 | 1.22 | 602 | 600 | C |
| C452 | 1.11 | 632 | 630 | C |
| C453 | 0.87 | 539 | 537 | C |
| C454 | 0.81 | 484 | 486 | C |
| C447a | 2.11 | 555 | 553 | D |
| C447b | 2.10 | 555 | 553 | D |
| C455a | 2.06 | 585 | 583 | D |
| C455b | 2.07 | 585 | 583 | D |
| C456 | 1.04 | 554 | 552 | C |
| C457 | 2.45 | 620 | 618 | L |
| C458 | 10.9 | 557 | | J |
| C452a | 2.02 | 632 | 630 | D |
| C452b | 2.03 | 632 | 630 | D |
| C460 | 1.10 | 539 | 539 | C |
| C410b | 2.09 | 620 | 618 | D |
| C410a | 2.09 | 620 | 618 | D |
| C461 | 2.19 | 588 | 586 | D |
| C462b | 1.83 | 630 | 628 | D |
| C459a | 2.15 | 570 | 568 | D |
| C459b | 2.16 | 570 | 568 | D |
| C464 | 1.42 | 641 | 643 | N |
| C465 | 1.403 | 658 | 660 | N |
| C466 | 1.389 | 630 | 632 | N |
| C467 | 1.509 | 566 | 568 | N |
| C468 | 1.217 | 641 | 643 | N |
| C469 | 1.213 | 579 | 581 | N |
| C470 | 1.347 | 568 | 570 | N |
| C471 | 1.433 | 591 | 593 | N |
| C472 | 1.359 | 544 | 546 | N |
| C473 | 1.263 | 532 | 534 | N |
| C474 | 1.079 | 513 | 515 | N |
| C475 | 1.85 | 560 | 558 | K |
| C476 | 1.4 | 562 | 564 | N |
| C477 | 1.322 | 512 | 514 | N |
| C478 | 1.331 | 510 | 512 | N |
| C479 | 1.249 | 593 | 595 | N |
| C480 | 1.376 | 582 | 584 | N |
| C481 | 1.18 | 593 | 595 | N |
| C482 | 1.326 | 582 | 584 | N |
| C483 | 1.068 | 605 | 607 | N |
| C484 | 1.244 | 593 | 595 | N |
| C485 | 1.186 | 640 | 642 | N |
| C486 | 1.352 | 641 | 643 | N |
| C487 | 1.446 | 639 | 641 | N |
| C488 | 1.187 | 654 | 656 | N |
| C489 | 1.282 | 593 | 595 | N |
| C490 | 1.346 | 621 | 623 | N |
| C491 | 1.288 | 589 | 591 | N |
| C492 | 1.318 | 490 | 492 | N |
| C493 | 1.266 | 530 | 532 | N |
| C494 | 1.01 | 513 | 515 | N |
| C495 | 1.041 | 578 | 580 | N |
| C496 | 1.108 | 472 | 474 | N |
| C497 | 0.989 | 498 | 500 | N |
| C498 | 1.347 | 528 | 530 | N |
| C499 | 1.313 | 490 | 492 | N |
| C500 | 1.311 | 526 | 528 | N |
| C501 | 1.363 | 504 | 506 | N |
| C502 | 1.294 | 494 | 496 | N |
| C503 | 1.018 | 515 | 517 | N |
| C504 | 1.329 | 522 | 524 | N |
| C505 | 1.347 | 515 | 517 | N |
| C506 | 1.301 | 511 | 513 | N |
| C507 | 1.294 | 472 | 474 | N |
| C508 | 1.286 | 517 | 519 | N |
| C509 | 1.059 | 528 | 530 | N |
| C510 | 1.067 | 528 | 530 | N |
| C511 | 1.343 | 556 | 558 | N |
| C512 | 1.358 | 586 | 588 | N |
| C513 | 1.331 | 568 | 570 | N |
| C514 | 1.366 | 506 | 508 | N |
| C515 | 1.321 | 490 | 492 | N |
| C516 | 1.392 | 541 | 543 | N |
| C517 | 1.287 | 593 | 595 | N |
| C518 | 1.37 | 582 | 584 | N |
| C519 | 1.368 | 574 | 576 | N |
| C520 | 1.335 | 510 | 512 | N |
| C521 | 1.382 | 602 | 604 | N |
| C522 | 1.354 | 604 | 606 | N |
| C523 | 1.382 | 602 | 604 | N |
| C524 | 1.364 | 544 | 546 | N |
| C525 | 2.01 | 578 | 576 | K |
| C526a | 2.04 | 618 | 616 | D |
| C526b | 2.04 | 618 | 616 | D |
| C527a | 2.04 | 648 | 646 | D |
| C527b | 2.04 | 648 | 646 | D |
| C528a | 1.78 | 647 | 645 | D |
| C528b | 1.83 | 647 | 645 | K |
| C529a | 1.81 | 630 | 628 | D |
| C529b | 1.81 | 630 | 628 | D |
| C530a | 1.76 | 647 | 645 | D |
| C530b | 1.75 | 647 | 645 | D |
| C531 | 1.20 | 662 | 664 | C |
| C532 | 1.33 | 667 | 665 | C |
| C533 | 2.23 | 659 | 657 | D |
| C534 | 1.21 | 662 | 660 | C |
| C535a | 2.14 | 644 | 642 | D |
| C535b | 2.14 | 644 | 642 | D |
| C536 | 2.00 | 576 | 574 | D |
| C537 | 1.24 | 675 | 675 | C |
| C538 | 1.289 | 641 | 643 | N |
| C539 | 1.259 | 647 | 649 | N |
| C540 | 1.275 | 593 | 595 | N |
| C541 | 1.142 | 579 | 581 | N |
| C542 | 1.176 | 641 | 643 | N |
| C543 | 1.373 | 641 | 643 | N |
| C544 | 1.171 | 639 | 641 | N |
| C545 | 1.216 | 607 | 609 | N |
| C546 | 1.342 | 655 | 657 | N |
| C547 | 1.475 | 552 | 554 | N |
| C548 | 1.498 | 659 | 661 | N |
| C549 | 1.404 | 594 | 596 | N |
| C550 | 1.495 | 594 | 596 | N |
| C551 | 1.126 | 607 | 609 | N |
| C552 | 1.245 | 629 | 631 | N |
| C553 | 1.291 | 641 | 643 | N |
| C554 | 1.345 | 615 | 617 | N |
| C555 | 1.285 | 641 | 643 | N |
| C556 | 1.328 | 641 | 643 | N |
| C557 | 1.155 | 579 | 581 | N |
| C558 | 1.416 | 582 | 584 | N |
| C559 | 1.252 | 554 | 556 | N |
| C560 | 1.241 | 579 | 581 | N |
| C561 | 1.191 | 579 | 581 | N |
| C562 | 1.124 | 664 | 666 | N |
| C563 | 1.378 | 538 | 540 | N |
| C564 | 1.362 | 529 | 531 | N |
| C565 | 1.354 | 572 | 574 | N |
| C566 | 1.448 | 565 | 567 | N |
| C567 | 1.326 | 541 | 543 | N |
| C568 | 1.412 | 595 | 597 | N |
| C569 | 1.393 | 577 | 579 | N |
| C570 | 1.356 | 529 | 531 | N |
| C571 | 1.346 | 604 | 606 | N |
| C572 | 1.379 | 595 | 597 | N |
| C573 | 1.318 | 522 | 524 | N |
| C574 | 1.337 | 574 | 576 | N |
| C575 | 1.412 | 596 | 598 | N |
| C576 | 1.516 | 611 | 613 | N |
| C577 | 1.393 | 590 | 592 | N |
| C578 | 1.404 | 566 | 568 | N |
| C579 | 1.425 | 548 | 550 | N |
| C580 | 1.389 | 590 | 592 | N |
| C581 | 1.355 | 570 | 572 | N |
| C582 | 1.335 | 526 | 528 | N |
| C583 | 1.445 | 594 | 596 | N |
| C584 | 1.377 | 570 | 572 | N |
| C585 | 1.363 | 540 | 542 | N |
| C586 | 1.353 | 552 | 554 | N |
| C587 | 1.405 | 581 | 583 | N |
| C588 | 1.346 | 594 | 596 | N |
| C589 | 1.358 | 552 | 554 | N |
| C590 | 1.347 | 556 | 558 | N |
| C591 | 1.355 | 583 | 585 | N |
| C592 | 1.371 | 590 | 592 | N |
| C593 | 1.3 | 522 | 524 | N |
| C594 | 1.381 | 549 | 551 | N |
| C595 | 1.147 | 593 | 595 | N |
| C596 | 1.582 | 662 | 664 | N |
| C597 | 1.357 | 641 | 643 | N |
| C598 | 1.352 | 633 | 635 | N |
| C599 | 1.167 | 609 | 611 | N |
| C600 | 1.108 | 581 | 583 | N |
| C601 | 1.34 | 647 | 649 | N |
| C602 | 1.441 | 606 | 608 | N |
| C603 | 1.517 | 655 | 657 | N |
| C604 | 1.118 | 546 | 548 | N |
| C605 | 1.526 | 646 | 648 | N |
| C606 | 1.83 | 520 | 518 | D |
| C625 | 1.09 | 543 | 541 | C |
| C607 | 1.17 | 664 | 662 | C |
| C608b | 1.23 | 672 | 670 | C |
| C609 | 1.12 | 640 | 638 | C |
| C610 | 1.01 | 538 | 536 | C |
| C608a | 1.21 | 672 | 670 | C |
| C611 | 1.93 | 538 | 536 | A |
| C612 | 1.25 | 671 | 669 | C |
| C613 | 1.18 | 624 | 622 | C |
| C614 | 1.14 | 610 | 608 | C |
| C615 | 1.21 | 660 | 658 | C |
| C616 | 1.27 | 687 | 685 | C |
| C617 | 2.29 | 676 | 674 | K |
| C618 | 2.15 | 678 | 676 | A |
| C619 | 1.22 | 662 | 660 | C |
| C620 | 2.26 | 678 | 676 | D |
| C621 | 1.26 | 622 | 620 | C |
| C622 | 2.23 | 678 | 676 | A |
| C623 | 2.15 | 662 | 660 | A |
| C624 | 1.12 | 612 | 610 | C |
| C626 | 1.56 | 491 | 489 | K |
| C627 | 8.70 | 540 | 540 | M |
| C628 | 1.13 | 674 | 672 | C |
| C630 | 1.11 | 648 | 646 | C |
| C631 | 1.16 | 624 | 622 | C |
| C632 | 2.28 | 680 | 678 | D |
| C633 | 1.11 | 608 | 606 | C |
| C634 | 1.79 | 528 | 526 | K |
| C635 | 2.01 | 578 | 576 | K |
| C636 | 2.42 | 680 | 678 | A |
| C637 | 2.08 | 543 | 545 | K |
| C638 | 2.06 | 569 | 567 | D |
| C639a | 2.04 | 596 | 594 | D |
| C639b | 2.06 | 596 | 594 | D |
| C640 | 1.21 | 664 | 662 | C |
| C641 | 1.65 | 503 | 501 | K |
| C642 | 2.05 | 596 | 594 | D |
| C643 | 1.98 | 592 | 590 | D |
| C644 | 2.52 | 664 | 662 | K |
| C645 | 1.03 | 566 | 564 | C |
| C646 | 0.96 | 532 | 530 | C |
| C643a | 1.99 | 592 | 590 | D |
| C647a | 1.70 | 630 | 628 | D |
| C647b | 1.69 | 630 | 628 | D |
| C648 | 1.94 | 562 | 560 | K |
| C643b | 1.99 | 592 | 590 | D |
| C649 | 2.02 | 618 | 616 | D |
| C650 | 2.28 | 692 | 690 | D |
| C651 | 2.12 | 642 | 640 | K |
| C652a | 2.31 | 692 | 690 | D |
| C652b | 2.32 | 692 | 690 | D |
| C653 | 0.84 | 525 | 523 | C |
| C654 | 1.10 | 588 | 586 | C |
| C655 | 1.18 | 659 | 657 | C |
| C656 | 2.17 | 506 | 504 | K |
| C657 | 1.06 | 542 | 540 | C |
| C659a | 2.23 | 676 | 674 | D |
| C659b | 2.29 | 676 | 674 | D |
| C660a | 2.10 | 640 | 638 | D |
| C660b | 2.09 | 640 | 638 | D |
| C661 | 2.13 | 602 | 600 | D |
| C662 | 2.01 | 602 | 600 | A |
| C661a | 2.13 | 602 | 600 | D |
| C661b | 2.14 | 602 | 600 | D |
| C663a | 2.26 | 676 | 674 | D |
| C663b | 2.27 | 676 | 674 | D |
| C664a | 2.30 | 692 | 690 | D |
| C664b | 2.32 | 692 | 690 | D |
| C665a | 1.91 | 626 | 624 | D |
| C665b | 1.85 | 626 | 624 | D |
| C666 | 2.17 | 526 | 524 | K |
| C667 | 2.24 | 516 | 514 | K |
| C668 | 1.99 | 566 | 564 | K |
| C669 | 2.09 | 612 | 642 | D |
| C669a | 2.12 | 612 | 610 | D |
| C669b | 2.10 | 612 | 610 | D |
| C670 | 1.05 | 617 | 615 | C |
| C671 | 1.06 | 566 | 564 | C |
| C672 | 0.95 | 548 | 546 | C |
| C673 | 2.17 | 622 | 620 | D |
| C674 | 1.82 | 529 | 527 | A |
| C675 | 1.65 | 512 | 510 | A |
| C676a | 2.19 | 622 | 620 | D |
| C676b | 2.21 | 622 | 620 | D |
| C677b | 2.23 | 610 | 608 | D |
| C678 | 2.09 | 618 | 616 | D |
| C679 | 1.91 | 560 | 558 | K |
| C680 | 1.71 | 518 | 516 | K |
| C681 | 1.02 | 570 | 568 | C |
| C682 | 1.86 | 600 | 598 | A |
| C683 | 1.07 | 563 | 561 | C |
| C684 | 1.05 | 564 | 562 | C |
| C673a | 1.14 | 622 | 620 | C |
| C673b | 1.15 | 622 | 620 | C |
| C685a | 1.96 | 578 | 576 | D |
| C685b | 2.04 | 578 | 576 | D |
| C686 | 1.77 | 519 | 517 | K |
| C687 | 0.99 | 552 | 550 | C |
| C688 | 2.16 | 632 | 628 | K |
| C678a | 2.11 | 618 | 616 | D |
| C678b | 2.11 | 618 | 616 | D |
| C689a | 2.17 | 626 | 624 | D |
| C689b | 2.18 | 626 | 624 | D |
| C690 | 1.09 | 568 | 566 | C |
| C691 | 2.63 | 701 | 699 | D |
| C692 | 1.01 | 552 | 550 | C |
| C693 | 1.01 | 532 | 530 | C |
| C694 | 2.22 | 592 | 590 | D |
| C695 | 2.06 | 564 | 562 | D |
| C696 | 2.22 | 568 | 566 | A |
| C697a | 2.25 | 643 | 641 | D |
| C697b | 2.20 | 643 | 641 | D |
| C677a | 2.21 | 610 | 608 | D |
| C698 | 1.96 | 563 | 561 | D |
| C699 | 2.06 | 570 | 568 | D |
| C700 | 1.10 | 592 | 590 | C |
| C701b | 2.04 | 620 | 618 | A |
| C701b | 2.06 | 620 | 618 | A |
| C702 | 0.92 | 538 | 416 | C |
| C703 | 1.15 | 594 | 592 | C |

¹H NMR data

| | NMR_SPECTRUM |
|---|---|
| C356 | 1H NMR (400 MHz, DMSO-d₆) δ ppm 1.06 (d, J = 6.6 Hz, 6 H) 2.46 (br t, |
| | J = 5.8 Hz, 2 H) 3.29 (s, 3 H) 3.73 (br t, J = 5.8 Hz, 2 H) 4.18 (dq, J = 13.7, |
| | 6.8 Hz, 1 H) 4.39 (s, 2 H) 5.54 (br d, J = 7.9 Hz, 1 H) 6.82 (dd, J = 9.3, 1.4 |
| | Hz, 1 H) 7.42 (s, 1 H) 7.49-7.58 (m, 2 H) 7.61 (d, J = 9.3 Hz, 1 H) 8.04- |
| | 8.14 (m, 2 H) 8.43 (s, 1 H) 8.58 (s, 1 H) |
| C357 | 1H NMR (400 MHz, DMSO-d₆) δ ppm 1.12 (d, J = 6.6 Hz, 6 H) 2.54 (t, J = |
| | 5.9 Hz, 2 H) 3.25 (s, 3 H) 3.95 (t, J = 5.9 Hz, 2 H) 4.12-4.25 (m, 1 H) |
| | 4.56 (t, J = 1.4 Hz, 2 H) 5.00 (br d, J = 7.6 Hz, 1 H) 6.86 (s, 1 H) 7.19 (dd, |
| | J = 8.7, 2.1 Hz, 1 H) 7.48 (d, J = 8.7 Hz, 1 H) 7.55 (d, J = 8.8 Hz, 2 H) 7.67 |
| | (d, J = 2.1 Hz, 1 H) 8.10 (d, J = 8.5 Hz, 2 H) 11.33 (br s, 1 H) |
| C358 | 1H NMR (400 MHz, DMSO-d₆) δ ppm 0.99-1.13 (m, 4 H) 2.45 (br t, J = |
| | 5.6 Hz, 1 H) 2.51-2.57 (m, 1 H) 3.27-3.36 (m, 4 H) 3.93 (br t, J = 5.8 |
| | Hz, 1 H) 4.16-4.33 (m, 1 H) 4.54 (s, 1 H) 4.62 (br t, J = 5.7 Hz, 1 H) 5.30 |
| | (s, 1 H) 5.72-5.85 (m, 1 H) 7.23-7.39 (m, 2 H) 7.50-7.62 (m, 3 H) 7.73- |
| | 7.85 (m, 1 H) 8.04-8.15 (m, 2 H) 13.14-13.25 (m, 1 H) |
| C359 | 1H NMR (400 MHz, DMSO-d₆, 126° C.) δ ppm 1.06 (d, J = 6.6 Hz, 6 H) |
| | 2.45 (br s, 1 H) 3.23 (s, 3 H) 3.73 (t, J = 5.9 Hz, 2 H) 4.13 (dq, J = 13.6, 6.7 |
| | Hz, 1 H) 4.38 (s, 2 H) 5.07 (br d, J = 7.5 Hz, 1 H) 7.25 (dd, J = 9.3, 1.7 Hz, |
| | 1 H) 7.52 (d, J = 8.5 Hz, 2 H) 7.57 (d, J = 9.3 Hz, 1 H) 7.60 (d, J = 1.3 Hz, 1 |
| | H) 7.94 (s, 1 H) 8.06 (d, J = 7.9 Hz, 2 H) 8.72 (dd, J = 1.8, 0.7 Hz, 1 H) |
| C360 | 1H NMR (400 MHz, DMSO-d₆, 101° C.) δ ppm 1.11 (d, J = 6.6 Hz, 6 H) |
| | 2.48 (s, 2 H) 3.27 (s, 3 H) 3.36-3.59 (m, 2 H) 3.69-3.91 (m, 2 H) 4.13- |
| | 4.27 (m, 1 H) 4.40 (s, 2 H) 5.26 (br d, J = 7.7 Hz, 1 H) 5.70 (dd, J = 9.9, |
| | 6.8 Hz, 1 H) 6.79 (d, J = 8.0 Hz, 1 H) 6.86 (td, J = 7.4, 1.0 Hz, 1 H) 7.11 |
| | (td, J = 7.7, 1.4 Hz, 1 H) 7.23 (dd, J = 7.4, 1.5 Hz, 1 H) 7.56 (d, J = 8.5 Hz, |
| | 2 H) 8.10 (d, J = 8.6 Hz, 2 H) |
| C361 | 1H NMR (400 MHz, DMSO-d₆, 101° C.) δ ppm 1.08 (d, J = 6.6 Hz, 5 H) |
| | 2.42-2.48 (m, 2 H) 3.27 (s, 3 H) 3.74 (br t, J = 5.8 Hz, 2 H) 4.09-4.24 |
| | (m, 1 H) 4.39 (s, 2 H) 5.26 (br d, J = 7.8 Hz, 1 H) 6.70 (d, J = 7.2 Hz, 1 H) |
| | 7.48-7.59 (m, 3 H) 7.70 (s, 1 H) 8.09 (d, J = 8.1 Hz, 2 H) 8.36 (d, J = 7.2 |
| | Hz, 1 H) 8.42 (s, 1 H) |
| C362 | ^{1H} NMR (400 MHz, DMSO-d₆, 150° C.) δ ppm 1.13 (d, J = 6.6 Hz, 6 H) |
| | 2.57 (t, J = 5.8 Hz, 2 H) 3.25 (s, 3 H) 4.04 (br t, J = 5.9 Hz, 2 H) 4.19 (dq, |
| | J = 13.4, 6.6 Hz, 1 H) 4.66 (s, 2 H) 4.97-5.07 (m, 1 H) 7.50 (td, J = 7.7, |
| | 1.1 Hz, 1 H) 7.56 (s, 3 H) 7.80 (d, J = 8.1 Hz, 1 H) 7.90 (d, J = 7.8 Hz, 1 H) |
| | 8.07-8.13 (m, 2 H) |
| C363 | 1H NMR (400 MHz, DMSO-d₆, 101° C.) δ ppm 1.07 (d, J = 6.6 Hz, 6 H) |
| | 2.45 (t, J = 5.8 Hz, 2 H) 3.27 (s, 3 H) 3.72 (t, J = 5.8 Hz, 2 H) 3.89 (s, 3 H) |
| | 4.15 (dq, J = 13.6, 6.8 Hz, 1 H) 4.39 (s, 2 H) 5.23 (br d, J = 7.7 Hz, 1 H) |
| | 7.31 (dd, J = 8.3, 1.6 Hz, 1 H) 7.55 (d, J = 8.6 Hz, 2 H) 7.66-7.69 (m, 1 H) |
| | 7.71 (d, J = 8.3 Hz, 1 H) 8.09 (d, J = 8.6 Hz, 2 H) 8.21 (s, 1 H) |
| C364 | 1H NMR (400 MHz, DMSO-d₆, 126° C.) δ ppm 1.09 (d, J = 6.6 Hz, 6 H) |
| | 2.46 (t, J = 5.9 Hz, 2 H) 3.26 (s, 3 H) 3.71 (t, J = 5.9 Hz, 2 H) 4.09-4.20 |
| | (m, 1 H) 4.38 (s, 2 H) 5.09 (br d, J = 7.7 Hz, 1 H) 7.51-7.58 (m, 3 H) 7.82 |
| | (d, J = 8.4 Hz, 1 H) 7.87 (d, J = 1.6 Hz, 1 H) 8.09 (d, J = 8.8 Hz, 2 H) 8.68 |
| | (s, 1 H) |
| C365 | 1H NMR (400 MHz, DMSO-d₆, 126° C.) δ ppm 1.09 (d, J = 6.6 Hz, 5 H) |
| | 2.44-2.49 (m, 2 H) 3.26 (s, 3 H) 3.73 (br t, J = 5.9 Hz, 2 H) 4.09-4.23 |
| | (m, 1 H) 4.41 (s, 2 H) 5.11 (br d, J = 7.7 Hz, 1 H) 7.22 (dd, J = 7.0, 1.7 Hz, |
| | 1 H) 7.55 (d, J = 8.6 Hz, 2 H) 7.89 (s, 1 H) 8.10 (d, J = 8.6 Hz, 2 H) 8.50 (s, |
| | 1 H) 8.94 (dd, J = 7.0, 1.0 Hz, 1 H) |
| C366 | 1H NMR (400 MHz, DMSO-d₆, 126° C.) δ ppm 1.09 (d, J = 6.6 Hz, 6 H) |
| | 2.42-2.49 (m, 2 H) 3.26 (s, 3 H) 3.73 (t, J = 5.8 Hz, 2 H) 4.15 (dq, J = |
| | 13.5, 6.7 Hz, 1 H) 4.41 (s, 2 H) 5.08 (br d, J = 7.7 Hz, 1 H) 7.52-7.60 (m, |
| | 3 H) 8.09 (d, J = 8.8 Hz, 2 H) 8.15 (d, J = 1.6 Hz, 1 H) 8.24 (d, J = 8.2 Hz, 1 |
| | H) 9.41 (s, 1 H) |
| C367 | 1H NMR (400 MHz, DMSO-d₆, 127° C.) δ ppm 1.10 (d, J = 6.6 Hz, 6 H) |
| | 2.45-2.49 (m, 1 H) 3.26 (s, 3 H) 3.69 (br t, J = 5.8 Hz, 2 H) 4.17 (dq, J = |
| | 13.5, 6.7 Hz, 1 H) 4.39 (s, 2 H) 5.12 (br d, J = 7.7 Hz, 1 H) 7.55 (d, J = 8.5 |
| | Hz, 2 H) 7.95 (d, J = 8.1 Hz, 1 H) 8.06-8.13 (m, 2 H) 8.17 (dd, J = 8.0, 2.1 |
| | Hz, 1 H) 8.85 (d, J = 2.1 Hz, 1 H) |
| C368 | 1H NMR (400 MHz, DMSO-d₆, 126° C.) δ ppm 1.11 (d, J = 6.6 Hz, 6 H) |
| | 2.54 (br t, J = 5.9 Hz, 3 H) 3.26 (s, 3 H) 3.96 (t, J = 5.9 Hz, 2 H) 4.19 (dq, |
| | J = 13.6, 6.7 Hz, 1 H) 4.58 (s, 2 H) 5.12 (br d, J = 7.6 Hz, 1 H) 6.96 (s, 1 H) |
| | 7.19 (dd, J = 8.3, 4.5 Hz, 1 H) 7.56 (d, J = 8.6 Hz, 2 H) 7.79-7.85 (m, 1 H) |
| | 8.10 (d, J = 8.6 Hz, 2 H) 8.42 (dd, J = 4.5, 1.5 Hz, 1 H) 11.37-11.54 (m, 1 |
| | H) |
| C369 | 1H NMR (400 MHz, DMSO-d₆, 151° C.) δ ppm 1.12 (d, J = 6.6 Hz, 6 H) |
| | 2.56 (t, J = 6.0 Hz, 2 H) 3.25 (s, 3 H) 3.96 (t, J = 5.9 Hz, 2 H) 4.13-4.25 |
| | (m, 1 H) 4.57 (s, 2 H) 4.93-5.01 (m, 1 H) 7.00 (s, 1 H) 7.40 (d, J = 5.8 |
| | Hz, 1 H) 7.52-7.59 (m, 2 H) 8.07-8.13 (m, 2 H) 8.25 (d, J = 5.8 Hz, 1 H) |
| | 8.93 (d, J = 1.2 Hz, 1 H) 11.44-11.56 (m, 1 H). |
| C370 | 1H NMR (400 MHz, DMSO-d₆, 100° C.) δ ppm 1.11 (d, J = 6.8 Hz, 3 H) |
| | 1.16-1.22 (m, 6 H) 1.53-1.65 (m, 1 H) 1.53-1.67 (m, 3 H) 1.97 (br d, |
| | J = 12.7 Hz, 2 H) 2.26 (s, 1 H) 2.34 (tt, J = 12.1, 3.5 Hz, 1 H) 2.41-2.48 |
| | (m, 1 H) 2.50-2.63 (m, 1 H) 3.60-3.64 (m, 3 H) 3.89 (br d, J = 18.5 Hz, 1 |
| | H) 4.14-4.21 (m, 1 H) 4.23 (br t, J = 11.9 Hz, 1 H) 4.42 (br s, 1 H) 4.45 |
| | (br s, 1 H) 6.18 (br d, J = 7.3 Hz, 1 H) 7.37 (dd, J = 8.2, 1.9 Hz, 1 H) 7.62 |
| | (d, J = 1.8 Hz, 1 H) 7.67 (d, J = 8.4 Hz, 1 H) |
| C371 | 1H NMR (400 MHz, DMSO-d₆, 100° C.) ä ppm 1.06 (dd, J = 6.5, 3.0 Hz, 6 |
| | H), 1.19 (d, J = 6.8 Hz, 3 H), 2.34 (d, J = 16.5 Hz, 1 H), 2.42-2.46 (m, 3 |
| | H), 2.50-2.59 (m, 1 H), 3.98-4.07 (m, 1 H), 4.08-4.21 (m, 1 H), 4.51 |
| | (br s, 2 H), 5.17 (br d, J = 7.7 Hz, 1 H), 7.41 (dd, J = 8.1, 2.0 Hz, 1 H), |
| | 7.47-7.53 (m, 2 H), 7.66 (s, 1 H), 7.69 (d, J = 8.5 Hz, 1 H), 8.22 (d, J = 8.6 Hz, |
| | 2 H) |
| C372 | 1H NMR (400 MHz, DMSO-d₆, 81° C.) δ ppm 1.13-1.23 (m, 3 H) 1.36- |
| | 1.45 (m, 3 H) 2.31 (br d, J = 16.5 Hz, 1 H) 2.50-2.59 (m, 1 H) 2.82 (d, J = |
| | 4.5 Hz, 3 H) 3.69-3.84 (m, 3 H) 3.89-4.14 (m, 1 H) 4.20-4.97 (m, 2 H) |
| | 5.33 (sxt, J = 6.8 Hz, 1 H) 5.74-5.83 (m, 1 H) 6.03-6.07 (m, 1 H) 7.16- |
| | 7.49 (m, 4 H) 7.63-7.75 (m, 2 H) 7.96 (d, J = 8.1 Hz, 2 H) 8.26-8.38 (m, |
| | 1 H) |
| C373 | 1H NMR (400 MHz, DMSO-d₆, 81° C.) δ ppm 1.18 (d, J = 6.8 Hz, 3 H) |
| | 1.48-1.65 (m, 1 H) 1.71-1.96 (m, 3 H) 2.01-2.12 (m, 2 H) 2.32 (d, J = |
| | 16.4 Hz, 1 H) 2.51-2.61 (m, 1 H) 2.81 (d, J = 4.6 Hz, 3 H) 2.83-3.27 (m, |
| | 4 H) 4.02 (br d, J = 18.9 Hz, 1 H) 4.22-4.83 (m, 2 H) 7.30-7.46 (m, 3 H) |
| | 7.68 (d, J = 2.0 Hz, 1 H) 7.70 (d, J = 8.1 Hz, 1 H) 7.90 (d, J = 8.5 Hz, 2 H) |
| | 8.24-8.36 (m, 1 H) |
| C374 | 1H NMR (400 MHz, DMSO-d₆) δ ppm 0.97-1.20 (m, 9 H) 1.22-1.26 (m, |
| | 1 H) 2.19-2.45 (m, 3 H) 2.67-2.82 (m, 3 H) 3.89 (br s, 1 H) 3.99-4.17 |
| | (m, 2 H) 5.75 (s, 1 H) 7.39-7.56 (m, 1 H) 7.67-7.90 (m, 2 H) 8.36 (br s, |
| | 1 H) |
| C375a | 1H NMR (400 MHz, DMSO-d₆, 81° C.) δ ppm 1.12 (d, J = 6.8 Hz, 3 H) |
| | 1.36 (d, J = 7.2 Hz, 3 H) 2.31 (br d, J = 16.4 Hz, 1 H) 2.50-2.58 (m, 1 H) |
| | 2.83 (d, J = 4.5 Hz, 3 H) 3.89 (br d, J = 17.6 Hz, 1 H) 4.18-4.83 (m, 2 H) |
| | 5.24 (quin, J = 7.2 Hz, 1 H) 5.97 (br d, J = 7.9 Hz, 1 H) 7.26 (d, J = 5.1 Hz, |
| | 2 H) 7.32-7.52 (m, 3 H) 7.66 (d, J = 2.0 Hz, 1 H) 7.70 (d, J = 8.1 Hz, 1 H) |
| | 8.00 (d, J = 8.4 Hz, 2 H) 8.27-8.38 (m, 1 H) 8.45 (d, J = 5.1 Hz, 2 H) |
| C375b | 1H NMR (400 MHz, DMSO-d₆, 81° C.) δ ppm 1.17 (d, J = 6.8 Hz, 3 H) |
| | 1.36 (d, J = 7.1 Hz, 3 H) 2.30 (br d, J = 16.1 Hz, 1 H) 2.50-2.58 (m, 1 H) |
| | 2.83 (d, J = 4.6 Hz, 3 H) 3.95 (br d, J = 18.8 Hz, 1 H) 4.19-4.73 (m, 2 H) |
| | 5.21 (quin, J = 7.2 Hz, 1 H) 5.98 (d, J = 7.7 Hz, 1 H) 7.24 (d, J = 5.0 Hz, 2 |
| | H) 7.32-7.48 (m, 3 H) 7.63 (d, J = 1.8 Hz, 1 H) 7.67 (d, J = 8.1 Hz, 1 H) |
| | 8.00 (d, J = 8.4 Hz, 2 H) 8.29-8.37 (m, 1 H) 8.44 (d, J = 5.2 Hz, 2 H) |
| C376 | 1H NMR (400 MHz, DMSO-d₆, 81° C.) δ ppm 1.04-1.10 (m, 6 H) 1.17 (d, |
| | J = 6.8 Hz, 3 H) 2.33 (br d, J = 16.2 Hz, 1 H) 2.51-2.61 (m, 1 H) 2.84 (d, |
| | J = 4.6 Hz, 3 H) 3.98-4.09 (m, 1 H) 4.18-4.72 (m, 3 H) 6.01 (d, J = 8.0 |
| | Hz, 1 H) 7.43 (dd, J = 8.2, 1.9 Hz, 1 H) 7.67-7.73 (m, 2 H) 8.04 (s, 2 H) |
| | 8.53-8.62 (m, 1 H) |
| C372b | 1H NMR (400 MHz, DMSO-d₆, 81° C.) δ ppm 1.16 (d, J = 6.8 Hz, 3 H) 1.39 |
| | (d, J = 6.9 Hz, 3 H) 2.31 (d, J = 16.5 Hz, 1 H) 2.50-2.60 (m, 1 H) 2.82 (d, |
| | J = 4.6 Hz, 3 H) 3.78 (s, 3 H) 4.01 (br d, J = 18.9 Hz, 1 H) 4.24-4.86 (m, 2 |
| | H) 5.34 (quin, J = 7.1 Hz, 1 H) 5.79 (br d, J = 8.0 Hz, 1 H) 6.06 (d, J = 1.9 |
| | Hz, 1 H) 7.21 (d, J = 1.9 Hz, 1 H) 7.33 (d, J = 8.0 Hz, 2 H) 7.42 (dd, J = 8.2, |
| | 1.9 Hz, 1 H) 7.68 (d, J = 2.0 Hz, 1 H) 7.71 (d, J = 8.1 Hz, 1 H) 7.97 (d, J = |
| | 8.2 Hz, 2 H) 8.26-8.37 (m, 1 H) |
| C372a | 1H NMR (400 MHz, DMSO-d₆, 81° C.) δ ppm 1.19 (d, J = 6.8 Hz, 3 H) 1.40 |
| | (d, J = 6.9 Hz, 3 H) 2.32 (d, J = 16.4 Hz, 1 H) 2.48-2.60 (m, 1 H) 2.82 (d, |
| | J = 4.6 Hz, 3 H) 3.75 (s, 3 H) 4.04 (br d, J = 18.8 Hz, 1 H) 4.18-4.91 (m, 2 |
| | H) 5.33 (quin, J = 6.9 Hz, 1 H) 5.80 (br d, J = 7.5 Hz, 1 H) 6.05 (d, J = 1.9 |
| | Hz, 1 H) 7.20 (d, J = 1.8 Hz, 1 H) 7.23-7.39 (m, 2 H) 7.40 (dd, J = 8.4, 2.0 |
| | Hz, 1 H) 7.67 (d, J = 2.0 Hz, 1 H) 7.70 (d, J = 8.4 Hz, 1 H) 7.96 (d, J = 8.3 |
| | Hz, 2 H) 8.27-8.37 (m, 1 H) |
| C377b | 1H NMR (400 MHz, DMSO-d₆, 81° C.) δ ppm 1.13 (d, J = 6.8 Hz, 3 H) |
| | 1.39 (d, J = 7.0 Hz, 3 H) 2.30 (d, J = 16.4 Hz, 1 H) 2.50-2.59 (m, 1 H) |
| | 2.83 (d, J = 4.5 Hz, 3 H) 3.92 (br d, J = 18.8 Hz, 1 H) 4.12-4.91 (m, 2 H) |
| | 5.28 (quin, J = 7.2 Hz, 1 H) 5.92 (br d, J = 7.8 Hz, 1 H) 7.27 (dd, J = 7.9, |
| | 4.7 Hz, 1 H) 7.38 (d, J = 8.4 Hz, 2 H) 7.41 (dd, J = 8.4, 2.0 Hz, 1 H) 7.64- |
| | 7.69 (m, 2 H) 7.70 (d, J = 8.1 Hz, 1 H) 7.99 (d, J = 8.3 Hz, 2 H) 8.29-8.36 |
| | (m, 1 H) 8.39 (dd, J = 4.7, 1.6 Hz, 1 H) 8.51 (d, J = 2.4 Hz, 1 H) |
| C378 | 1H NMR (400 MHz, DMSO-d₆, 81° C.) δ ppm 1.16 (d, J = 6.8 Hz, 3 H) |
| | 2.31 (br d, J = 16.3 Hz, 1 H) 2.51-2.59 (m, 1 H) 2.82 (d, J = 4.5 Hz, 3 H) |
| | 3.97 (br d, J = 18.9 Hz, 1 H) 4.18-4.85 (m, 4 H) 6.40-6.53 (m, 1 H) 7.26 |
| | (dd, J = 7.8, 4.7 Hz, 1 H) 7.29-7.47 (m, 3 H) 7.59-7.65 (m, 1 H) 7.66 (d, |
| | J = 1.8 Hz, 1 H) 7.69 (d, J = 8.1 Hz, 1 H) 7.99 (d, J = 8.5 Hz, 2 H) 8.25- |
| | 8.36 (m, 1 H) 8.39 (dd, J = 4.8, 1.7 Hz, 1 H) 8.43-8.49 (m, 1 H) |
| C377a | 1H NMR (400 MHz, DMSO-d₆, 81° C.) δ ppm 1.17 (d, J = 6.8 Hz, 3 H) |
| | 1.39 (d, J = 7.1 Hz, 3 H) 2.30 (d, J = 16.4 Hz, 1 H) 2.46-2.57 (m, 1 H) |
| | 2.83 (d, J = 4.5 Hz, 3 H) 3.98 (br d, J = 18.9 Hz, 1 H) 4.12-4.77 (m, 2 H) |
| | 5.24 (quin, J = 7.2 Hz, 1 H) 5.93 (d, J = 7.7 Hz, 1 H) 7.26 (dd, J = 7.9, 4.7 |
| | Hz, 1 H) 7.30-7.44 (m, 3 H) 7.59-7.74 (m, 3 H) 8.00 (d, J = 8.4 Hz, 2 H) |
| | 8.28-8.36 (m, 1 H) 8.39 (dd, J = 4.7, 1.7 Hz, 1 H) 8.49 (d, J = 2.3 Hz, 1 H) |
| C379 | 1H NMR (400 MHz, DMSO-d₆, 100° C.) δ ppm 1.18 (d, J = 6.8 Hz, 3 H) |
| | 1.22 (dd, J = 6.6, 3.5 Hz, 6 H) 2.39 (d, J = 16.3 Hz, 1 H) 2.55-2.64 (m, 1 |
| | H) 2.82 (d, J = 4.6 Hz, 3 H) 4.03 (br d, J = 18.9 Hz, 1 H) 4.19 (dq, J = 13.2, |
| | 6.6 Hz, 1 H) 4.40-4.65 (m, 2 H) 7.41 (dd, J = 8.2, 2.0 Hz, 1 H) 7.66 (d, J = |
| | 2.0 Hz, 1 H) 7.69 (d, J = 8.1 Hz, 1 H) 8.00 (s, 1 H) 8.26 (s, 1 H) 8.46 (br d, |
| | J = 7.1 Hz, 1 H) |
| C380 | 1H NMR (400 MHz, DMSO-d₆, 81° C.) δ ppm 1.17 (d, J = 6.8 Hz, 3 H) |
| | 1.35 (d, J = 7.0 Hz, 3 H) 2.30 (d, J = 16.4 Hz, 1 H) 2.49-2.56 (m, 1 H) |
| | 2.83 (d, J = 4.6 Hz, 3 H) 3.98 (br d, J = 18.9 Hz, 1 H) 4.21-4.76 (m, 2 H) |
| | 5.20 (quin, J = 7.2 Hz, 1 H) 5.76 (br d, J = 7.8 Hz, 1 H) 6.99-7.09 (m, 2 H) |
| | 7.25-7.46 (m, 5 H) 7.64 (d, J = 2.0 Hz, 1 H) 7.68 (d, J = 8.1 Hz, 1 H) 7.99 |
| | (d, J = 8.4 Hz, 2 H) 8.28-8.38 (m, 1 H) |
| I70 | 1H NMR (400 MHz, DMSO-d₆, 80° C.) δ ppm 1.00-1.09 (m, 6 H) 1.18 (d, |
| | J = 6.7 Hz, 3 H) 2.32 (br d, J = 16.4 Hz, 1 H) 2.50-2.59 (m, 1 H) 4.02 (br |
| | d, J = 18.8 Hz, 1 H) 4.15 (dq, J = 13.6, 6.8 Hz, 1 H) 4.25-4.91 (m, 2 H) |
| | 5.17 (br d, J = 7.9 Hz, 1 H) 7.37 (br d, J = 8.4 Hz, 2 H) 7.42 (dd, J = 8.3, 1.9 |
| | Hz, 1 H) 7.68 (d, J = 1.9 Hz, 1 H) 7.70 (d, J = 8.1 Hz, 1 H) 8.08 (d, J = 8.7 |
| | Hz, 2 H) 12.42-13.27 (m, 1 H) |
| C381 | 1H NMR (400 MHz, DMSO-d₆) δ ppm −0.03-0.02 (m, 2 H) 0.15-0.25 |
| | (m, 2 H) 0.78 (s, 3 H) 2.17 (br d, J = 16.5 Hz, 1 H) 2.35-2.43 (m, 1 H) |
| | 2.68 (d, J = 4.6 Hz, 3 H) 2.96 (dd, J = 13.5, 5.4 Hz, 2 H) 3.02-3.12 (m, 1 |
| | H) 3.84 (br d, J = 19.4 Hz, 1 H) 4.34 (br s, 1 H) 5.22 (br t, J = 5.9 Hz, 1 H) |
| | 7.21 (br d, J = 8.1 Hz, 2 H) 7.27 (dd, J = 8.1, 2.0 Hz, 1 H) 7.53 (d, J = 2.0 |
| | Hz, 1 H) 7.55 (d, J = 8.4 Hz, 1 H) 7.85 (d, J = 8.8 Hz, 2 H) 8.18 (br d, J = |
| | 4.4 Hz, 1 H) |
| C382 | 1H NMR (400 MHz, CHLOROFORM-d, 27° C.) δ ppm 1.02-1.36 (m, 15 |
| | H) 2.44-2.60 (m, 1 H) 2.62-2.83 (m, 1 H) 3.92-4.40 (m, 4 H) 4.50 (br |
| | d, J = 7.0 Hz, 1 H) 4.92-5.56 (m, 1 H) 7.05-7.22 (m, 1 H) 7.24-7.30 (m, |
| | 1 H) 7.46-7.61 (m, 2 H) 9.22 (s, 2 H) |
| C383a | 1H NMR (400 MHz, DMSO-d₆, 81° C.) δ ppm 0.99-1.07 (m, 6 H) 1.12 (t, |
| | J = 7.6 Hz, 3H) 1.18 (d, J = 6.8 Hz, 3 H) 2.28-2.38 (m, 3 H) 2.51-2.60 |
| | (m, 1 H) 2.83 (d, J = 4.6 Hz, 3 H) 4.02 (br d, J = 18.8 Hz, 1 H) 4.16 (dsxt, |
| | J = 13.6, 6.8, 6.8, 6.8, 6.8, 6.8 Hz, 1 H) 4.24-4.84 (m, 2 H) 5.02 (br d, J = |
| | 8.0 Hz, 1 H) 7.22 (d, J = 8.1 Hz, 1 H) 7.44 (dd, J = 8.2, 1.9 Hz, 1 H) 7.69 |
| | (d, J = 1.5 Hz, 1 H) 7.70 (d, J = 4.8 Hz, 1 H) 7.80 (dd, J = 8.1, 2.0 Hz, 1 H) |
| | 7.90 (d, J = 2.0 Hz, 1 H) 8.26-8.35 (m, 1 H) |
| C383b | 1H NMR (400 MHz, DMSO-d₆, 81° C.) δ ppm 0.98-1.07 (m, 6 H) 1.11 (t, |
| | J = 7.6 Hz, 3H) 1.18 (d, J = 6.8 Hz, 3 H) 2.28-2.40 (m, 3 H) 2.50-2.58 |
| | (m, 1 H) 2.83 (d, J = 4.5 Hz, 3 H) 4.04 (br d, J = 18.7 Hz, 1 H) 4.17 (dq, J = |
| | 13.6, 6.8 Hz, 1 H) 4.24-4.89 (m, 2 H) 5.05 (br d, J = 8.0 Hz, 1 H) 7.19 (d, |
| | J = 8.1 Hz, 1 H) 7.42 (dd, J = 8.2, 1.9 Hz, 1 H) 7.68 (d, J = 2.0 Hz, 1 H) |
| | 7.70 (d, J = 8.1 Hz, 1 H) 7.80 (dd, J = 8.1, 2.0 Hz, 1 H) 7.90 (d, J = 2.0 Hz, |
| | 1 H) 8.25-8.36 (m, 1 H) |
| C384 | 1H NMR (400 MHz, DMSO-d₆, 81° C.) δ ppm 1.07-1.17 (m, 9 H) 2.24- |
| | 2.34 (m, 1 H) 2.40-2.50 (m, 1 H) 3.81-4.04 (m, 2 H) 4.19-4.67 (m, 2 |
| | H) 6.03 (br d, J = 7.4 Hz, 1 H) 7.39 (dd, J = 8.2, 2.0 Hz, 1 H) 7.65 (d, J = |
| | 1.9 Hz, 1 H) 7.69 (d, J = 8.2 Hz, 1 H) 9.94-10.48 (m, 1 H) |
| C385 | 1H NMR (400 MHz, ACETONITRILE-d3, 80° C.) ä ppm 1.14 (dd, J = 6.6, |
| | 2.0 Hz, 6 H), 1.27 (d, J = 6.8 Hz, 3 H), 2.43 (d, J = 16.1 Hz, 1 H), 2.60- |
| | 2.68 (m, 1 H), 3.27 (s, 3 H), 4.05 (br d, J = 18.3 Hz, 1 H), 4.19-4.31 (m, 1 |
| | H), 4.47 (br d, J = 7.7 Hz, 1 H), 4.61 (br s, 2 H), 7.38 (dd, J = 8.1, 2.0 Hz, 1 |
| | H), 7.68 (t, J = 54.6 Hz, 1 H), 7.63 (d, J = 1.8 Hz, 1 H), 7.65 (d, J = 8.1 Hz, |
| | 1 H), 7.71 (dt, J = 8.4, 1.1 Hz, 1 H), 7.86 (s, 1 H), 8.29 (d, J = 8.4 Hz, 1 H) |
| C386a | 1H NMR (400 MHz, DMSO-d₆, 101° C.) δ ppm 1.19 (d, J = 6.8 Hz, 3 H) |
| | 1.40 (d, J = 7.0 Hz, 3 H) 2.33 (br d, J = 16.3 Hz, 1 H) 2.53-2.62 (m, 2 H) |
| | 2.86 (d, J = 4.6 Hz, 3 H) 3.99 (br d, J = 19.0 Hz, 1 H) 4.31-4.62 (m, 2 H) |
| | 5.27 (quin, J = 7.3 Hz, 1 H) 6.03 (br d, J = 7.8 Hz, 1 H) 7.15-7.21 (m, 1 H) |
| | 7.22-7.33 (m, 4 H) 7.40 (dd, J = 8.2, 2.0 Hz, 1 H) 7.57 (d, J = 8.2 Hz, 1 H) |
| | 7.64-7.72 (m, 2 H) 7.96 (dd, J = 8.2, 1.9 Hz, 1 H) 8.09 (d, J = 1.9 Hz, 1 H) |
| | 8.40 (br d, J = 5.5 Hz, 1 H) |
| C386b | 1H NMR (400 MHz, DMSO-d₆, 101° C.) δ ppm 1.20 (d, J = 6.8 Hz, 3 H) |
| | 1.40 (d, J = 7.0 Hz, 3 H) 2.35 (br d, J = 16.3 Hz, 1 H) 2.54-2.61 (m, 1 H) |
| | 2.85 (d, J = 4.6 Hz, 3 H) 4.03 (br d, J = 18.9 Hz, 1 H) 4.33-4.57 (m, 1 H) |
| | 5.30 (quin, J = 7.2 Hz, 1 H) 6.06 (br d, J = 7.9 Hz, 1 H) 7.19 (s, 1 H) 7.23- |
| | 7.32 (m, 4 H) 7.39 (dd, J = 8.2, 2.0 Hz, 1 H) 7.49 (d, J = 8.2 Hz, 1 H) 7.64 |
| | (d, J = 1.9 Hz, 1 H) 7.68 (d, J = 8.2 Hz, 1 H) 7.95 (dd, J = 8.2, 1.9 Hz, 1 H) |
| | 8.10 (d, J = 1.9 Hz, 1 H) 8.40 (br d, J = 5.6 Hz, 1 H) |
| C386 | 1H NMR (400 MHz, DMSO-d₆, 126° C.) δ ppm 1.20 (dd, J = 6.8, 1.4 Hz, 3 |
| | H) 1.38-1.44 (m, 3 H) 2.36 (dd, J = 16.4, 5.1 Hz, 1 H) 2.52-2.61 (m, 1 |
| | H) 2.82-2.91 (m, 5 H) 3.95-4.08 (m, 1 H) 4.38-4.55 (m, 2 H) 5.27 (dt, |
| | J = 9.9, 7.2 Hz, 1 H) 5.84 (br t, J = 7.6 Hz, 1 H) 7.14-7.22 (m, 1 H) 7.23- |
| | 7.32 (m, 4 H) 7.39 (ddd, J = 8.2, 5.0, 1.9 Hz, 1 H) 7.52 (dd, J = 34.6, 8.2 |
| | Hz, 1 H) 7.61-7.70 (m, 2 H) 7.96 (ddd, J = 8.2, 3.4, 1.9 Hz, 1 H) 8.10 (dd, |
| | J = 5.4, 1.9 Hz, 1 H) 8.30 (br d, J = 5.5 Hz, 1 H) |
| C387a | 1H NMR (400 MHz, DMSO-d₆, 101° C.) δ ppm 0.13-0.44 (m, 4 H) 0.93 |
| | (qt, J = 8.2, 5.0 Hz, 1 H) 1.15 (d, J = 6.7 Hz, 3 H) 1.19 (d, J = 6.8 Hz, 3 H) |
| | 2.34 (d, J = 16.3 Hz, 1 H) 2.58 (br dd, J = 16.3, 6.1 Hz, 1 H) 2.86 (d, J = 4.6 |
| | Hz, 3 H) 3.52-3.68 (m, 1 H) 3.99 (br d, J = 18.9 Hz, 1 H) 4.33-4.71 (m, |
| | 2 H) 5.42 (br d, J = 8.0 Hz, 1 H) 7.44 (dd, J = 8.2, 2.0 Hz, 1 H) 7.53 (d, J = |
| | 8.2 Hz, 1 H) 7.67-7.73 (m, 2 H) 7.95 (dd, J = 8.2, 1.9 Hz, 1 H) 8.10 (d, J = |
| | 1.9 Hz, 1 H) 8.40 (br d, J = 5.3 Hz, 1 H) |
| C387b | 1H NMR (400 MHz, DMSO-d₆, 81° C.) δ ppm 0.12-0.44 (m, 4 H) 0.87- |
| | 0.99 (m, 1 H) 1.14 (d, J = 6.7 Hz, 3 H) 1.18 (d, J = 6.8 Hz, 3 H) 2.34 (br d, |
| | J = 16.3 Hz, 1 H) 2.51-2.60 (m, 1 H) 2.85 (d, J = 4.6 Hz, 3 H) 3.45-3.58 |
| | (m, 1 H) 4.02 (br d, J = 18.9 Hz, 1 H) 4.24-4.75 (m, 1 H) 5.61 (br d, J = |
| | 8.0 Hz, 1 H) 7.43 (dd, J = 8.2, 1.9 Hz, 1 H) 7.50 (d, J = 8.2 Hz, 1 H) 7.69 |
| | (d, J = 1.9 Hz, 1 H) 7.72 (d, J = 8.2 Hz, 1 H) 7.96 (dd, J = 8.2, 1.9 Hz, 1 H) |
| | 8.10 (d, J = 1.9 Hz, 1 H) 8.48 (br d, J = 5.3 Hz, 1 H) |
| C387 | 1H NMR (400 MHz, DMSO-d₆, 150° C.) δ ppm 0.13-0.46 (m, 4 H) 0.87-0.99 (m, 1 H) |
| | 1.16 (dd, J = 6.6, 2.0 Hz, 3 H) 1.21 (dd, J = 6.8, 1.6 Hz, 3 H) |
| | 2.34-2.43 (m, 1 H) 2.54-2.65 (m, 1 H) 2.87 (d, J = 4.6 Hz, 3 H) 3.53- |
| | 3.67 (m, 1 H) 3.96-4.08 (m, 1 H) 4.44-4.63 (m, 2 H) 5.03-5.15 (m, 1 |
| | H) 7.37-7.45 (m, 1 H) 7.46-7.54 (m, 1 H) 7.65 (dd, J = 5.7, 1.9 Hz, 1 H) |
| | 7.67-7.72 (m, 1 H) 7.92-7.99 (m, 1 H) 8.07-8.12 (m, 1 H) 8.21 (s, 1 H) |
| C388 | 1H NMR (400 MHz, CHLOROFORM-D δ ppm 1.05-1.28 (m, 16 H) 2.47- |
| | 2.56 (m, 1 H) 2.65 (br s, 1 H) 3.57 (dq, J = 13.4, 6.6 Hz, 1 H) 3.70 (br d, |
| | J = 7.7 Hz, 1 H) 3.99-4.26 (m, 2 H) 4.45 (d, J = 7.7 Hz, 1 H) 5.30 (s, 1 H) |
| | 7.27-7.45 (m, 3 H) 7.53 (d, J = 7.3 Hz, 1 H) 7.54 (s, 1 H) 8.08 (d, J = 9.0 |
| | Hz, 2 H) |
| C389 | 1H NMR (400 MHz, DMSO-d₆, 100° C.) δ ppm 1.17 (d, J = 6.8 Hz, 3 H) |
| | 2.30-2.46 (m, 1 H) 2.50-2.65 (m, 1 H) 2.83 (d, J = 4.6 Hz, 3 H) 3.44-3.56 |
| | (m, 3 H) 3.95 (br d, J = 18.9 Hz, 1 H) 4.48 (br s, 2 H) 6.01 (br s, 1 H) |
| | 7.24-7.29 (m, 1 H) 7.38 (dd, J = 8.3, 1.9 Hz, 1 H) 7.48 (br d, J = 8.1 Hz, 2 |
| | H) 7.64 (d, J = 2.0 Hz, 1 H) 7.67 (d, J = 8.4 Hz, 2 H) 7.99 (br d, J = 8.1 Hz, |
| | 2 H) 8.13-8.28 (m, 1 H) |
| C390 | 1H NMR (400 MHz, DMSO-d₆, 81° C.) δ ppm 0.67-0.81 (m, 6 H) 0.99 (d, |
| | J = 6.7 Hz, 3 H) 1.18 (d, J = 6.8 Hz, 3 H) 1.66 (dq, J = 13.5, 6.8 Hz, 1 H) |
| | 2.32 (br d, J = 16.3 Hz, 1 H) 2.50-2.58 (m, 1 H) 2.83 (d, J = 4.6 Hz, 3 H) |
| | 3.87 (sxt, J = 6.9 Hz, 1 H) 4.01 (br d, J = 18.9 Hz, 1 H) 4.25-4.68 (m, 2 H) |
| | 4.75 (br d, J = 8.4 Hz, 1 H) 7.36 (br d, J = 8.0 Hz, 2 H) 7.42 (dd, J = 8.2, 2.0 |
| | Hz, 1 H) 7.68 (d, J = 1.9 Hz, 1 H) 7.70 (d, J = 8.2 Hz, 1 H) 7.99 (d, J = 8.5 |
| | Hz, 2 H) 8.30-8.38 (m, 1 H) |
| C391 | 1H NMR (400 MHz, DMSO-d₆, 81° C.) δ ppm 1.00-1.08 (m, 6 H) 1.17 (d, |
| | J = 6.8 Hz, 3 H) 2.00 (s, 3 H) 2.02 (s, 3 H) 2.35 (br d, J = 16.4 Hz, 1 H) |
| | 2.52-2.62 (m, 1 H) 2.81 (d, J = 4.5 Hz, 3 H) 4.03 (br d, J = 18.5 Hz, 1 H) |
| | 4.19 (dq, J = 13.7, 6.9 Hz, 1 H) 4.28-4.82 (m, 2 H) 5.18 (br d, J = 8.1 Hz, |
| | 1 H) 7.41-7.45 (m, 1 H) 7.65-7.72 (m, 4 H) 8.17-8.26 (m, 1 H) |
| C392a | 1H NMR (400 MHz, DMSO-d₆) δ ppm 1.04-1.12 (m, 7 H) 1.07 (s, 1 H) |
| | 1.15-1.21 (m, 3 H) 1.18 (s, 1 H) 2.28-2.39 (m, 1 H) 2.50-2.61 (m, 1 H) |
| | 3.80-3.91 (m, 3 H) 3.85 (s, 1 H) 3.96-4.11 (m, 1 H) 4.14-4.27 (m, 1 H) |
| | 4.31-4.71 (m, 2 H) 5.50-5.58 (m, 1 H) 7.01-7.06 (m, 1 H) 7.26-7.29 |
| | (m, 1 H) 7.39-7.53 (m, 2 H) 7.66-7.73 (m, 2 H) 7.79-7.85 (m, 1 H) |
| | 7.94-7.99 (m, 1 H) |
| C393 | 1H NMR (400 MHz, DMSO-d₆) δ ppm 1.00-1.10 (m, 6 H) 1.13-1.23 (m, |
| | 3H) 1.19 (s, 1 H) 1.23-1.39 (m, 1 H) 1.49-1.61 (m, 1 H) 1.98 (s, 1 H) |
| | 2.27-2.39 (m, 1 H) 2.50-2.62 (m, 1 H) 3.94-4.10 (m, 1 H) 4.12-4.27 |
| | (m, 1 H) 4.32-4.73 (m, 2 H) 5.45-5.59 (m, 1 H) 7.04-7.16 (m, 1 H) |
| | 7.22-7.33 (m, 1 H) 7.38-7.49 (m, 2 H) 7.41 (s, 1 H) 7.64-7.76 (m, 2 H) |
| | 8.00-8.06 (m, 1 H) 8.15-8.22 (m, 1 H) 12.58 (br s, 1 H) |
| C346b | 1H NMR (400 MHz, DMSO-d₆) δ ppm 1.18 (d, J = 6.6 Hz, 3 H) 1.55-1.82 |
| | (m, 7 H) 1.85-1.96 (m, 2 H) 2.11-2.22 (m, 1 H) 2.32 (d, J = 16.3 Hz, 1 |
| | H) 2.51-2.62 (m, 2 H) 2.83 (d, J = 4.6 Hz, 3 H) 4.01 (br d, J = 19.1 Hz, 1 |
| | H) 4.18-4.29 (m, 1 H) 4.37-4.66 (m, 1 H) 5.23 (d, J = 7.0 Hz, 1 H) 7.37- |
| | 7.43 (m, 3 H) 7.67 (d, J = 1.8 Hz, 1 H) 7.69 (d, J = 8.1 Hz, 1 H) 8.00 (d, J = |
| | 7.8 Hz, 2 H) 8.33 (br d, J = 4.6 Hz, 1 H) |
| C394a | 1H NMR (400 MHz, DMSO-d₆) δ ppm 1.14 (d, J = 6.8 Hz, 3 H) 1.34 (d, J = |
| | 7.0 Hz, 3 H) 2.31 (br d, J = 16.5 Hz, 1 H) 2.54 (br dd, J = 16.7, 6.2 Hz, 1 H) |
| | 2.83 (d, J = 4.6 Hz, 3 H) 3.72 (s, 3 H) 3.95 (br d, J = 18.3 Hz, 1 H) 4.50 (br |
| | s, 2 H) 5.22 (br t, J = 7.3 Hz, 1 H) 5.68 (d, J = 7.9 Hz, 1 H) 6.71-6.78 (m, |
| | 1 H) 6.79-6.87 (m, 1 H) 7.18 (t, J = 7.8 Hz, 1 H) 7.37 (d, J = 8.4 Hz, 2 H) |
| | 7.41 (dd, J = 8.3, 1.9 Hz, 1 H) 7.68 (d, J = 2.0 Hz, 1 H) 7.70 (d, J = 8.4 Hz, |
| | 1 H) 8.00 (d, J = 8.6 Hz, 2 H) 8.29-8.38 (m, 1 H) |
| C395 | 1H NMR (400 MHz, DMSO-d₆, 100° C.) δ ppm 1.04 (dd, J = 6.6, 2.9 Hz, 6 |
| | H) 1.20 (d, J = 6.8 Hz, 3 H) 2.34 (d, J = 16.4 Hz, 1 H) 2.51-2.59 (m, 1 H) |
| | 2.60 (s, 3 H) 4.03 (br d, J = 18.7 Hz, 1 H) 4.16 (dq, J = 13.6, 6.8 Hz, 1 H) |
| | 4.39-4.65 (m, 2 H) 5.21 (br d, J = 7.8 Hz, 1 H) 7.20 (d, J = 8.3 Hz, 1 H) |
| | 7.41 (dd, J = 8.2, 2.0 Hz, 1 H) 7.66 (d, J = 2.0 Hz, 2 H) 7.69 (d, J = 8.1 Hz, |
| | 1 H) 7.96 (d, J = 8.1 Hz, 1 H) |
| C396 | 1H NMR (400 MHz, DMSO-d₆, 81° C.) δ ppm 0.11-0.28 (m, 4 H) 1.10- |
| | 1.20 (m, 4 H) 1.22 (s, 6 H) 2.33 (d, J = 16.5 Hz, 1 H) 2.52-2.61 (m, 1 H) |
| | 2.83 (d, J = 4.6 Hz, 3 H) 4.02 (br d, J = 18.9 Hz, 1 H) 4.11 (s, 1 H) 4.25- |
| | 4.94 (m, 2 H) 7.36-7.46 (m, 3 H) 7.68 (d, J = 2.0 Hz, 1 H) 7.70 (d, J = 8.4 |
| | Hz, 1 H) 8.01 (d, J = 8.5 Hz, 2 H) 8.31-8.42 (m, 1 H) |
| C346a | 1H NMR (400 MHz, DMSO-d₆) δ ppm 1.18 (d, J = 6.6 Hz, 3 H) 1.58-1.85 |
| | (m, 8 H) 1.87-1.96 (m, 2 H) 2.16-2.23 (m, 1 H) 2.30-2.39 (m, 1 H) |
| | 2.51-2.58 (m, 3 H) 2.83 (d, J = 4.6 Hz, 3 H) 3.96-4.04 (m, 1 H) 4.17- |
| | 4.24 (m, 1 H) 4.39-4.62 (m, 1 H) 5.18 (d, J = 6.6 Hz, 1 H) 7.37-7.44 (m, |
| | 3 H) 7.68 (d, J = 2.0 Hz, 1 H) 7.70 (d, J = 8.1 Hz, 1 H) 8.00 (d, J = 8.6 Hz, 2 |
| | H) 8.31-8.36 (m, 1 H) |
| C394b | 1H NMR (400 MHz, DMSO-d₆) δ ppm 1.18 (d, J = 6.8 Hz, 3 H) 1.34 (d, J = |
| | 7.0 Hz, 3 H) 2.27-2.35 (m, 1 H) 2.51-2.57 (m, 1 H) 2.83 (d, J = 4.4 Hz, 3 |
| | H) 3.70 (s, 3 H) 3.95-4.08 (m, 1 H) 4.32-4.57 (m, 1 H) 5.17 (quin, J = |
| | 7.2 Hz, 1 H) 5.70 (d, J = 7.5 Hz, 1 H) 6.72-6.76 (m, 1 H) 6.79-6.84 (m, 2 |
| | H) 7.16 (t, J = 8.1 Hz, 1 H) 7.34-7.43 (m, 2 H) 7.65 (d, J = 2.0 Hz, 1 H) |
| | 7.68 (d, J = 8.1 Hz, 1 H) 8.00 (d, J = 8.0 Hz, 2 H) 8.31-8.37 (m, 1 H) |
| C397 | ¹H NMR (400 MHz, DMSO-d₆) δ ppm 0.69-0.80 (m, 3 H) 1.21 (br dd, |
| | J = 18.19, 6.32 Hz, 6 H) 1.41-1.55 (m, 2 H) 2.31-2.42 (m, 1 H) 2.63 (br |
| | d, J = 15.92 Hz, 1 H) 2.80-2.89 (m, 3 H) 4.05-4.18 (m, 1 H) 4.48-4.72 |
| | (m, 2 H) 5.05-5.14 (m, 1 H) 7.31-7.38 (m, 2 H) 7.41-7.48 (m, 1 H) |
| | 7.65-7.76 (m, 2 H) 7.87-7.96 (m, 2 H) 8.11-8.33 (m, 1 H) |
| C398 | ¹H NMR (400 MHz, DMSO-d₆) δ ppm 0.73 (t, J = 7.45 Hz, 3 H) 1.16-1.24 |
| | (m, 6 H) 1.41-1.56 (m, 2 H) 2.35-2.43 (m, 1 H) 2.57-2.69 (m, 1 H) |
| | 2.85 (d, J = 4.80 Hz, 3 H) 4.03-4.18 (m, 1 H) 4.44-4.68 (m, 2 H) 5.02- |
| | 5.15 (m, 1 H) 7.34 (d, J = 8.59 Hz, 2 H) 7.45 (dd, J = 8.21, 1.89 Hz, 1 H) |
| | 7.66-7.77 (m, 2 H) 7.88-7.96 (m, 2 H) 8.20 (br d, J = 2.53 Hz, 1 H) |
| C399 | 1H NMR (400 MHz, DMSO-d₆) δ ppm 0.99-1.10 (m, 6 H), 1.16-1.22 |
| | (m, 3 H), 2.05 (s, 3 H), 2.28-2.42 (m, 1 H), 2.51-2.63 (m, 1 H), 3.95- |
| | 4.10 (m, 1 H), 4.10-4.24 (m, 1 H), 4.30-4.75 (m, 2 H), 5.04-5.13 (m, 1 |
| | H), 7.01-7.11 (m, 1 H), 7.14-7.26 (m, 2 H), 7.40-7.46 (m, 1 H), 7.66- |
| | 7.74 (m, 2 H), 7.86-7.92 (m, 1 H), 7.96-8.02 (m, 1 H), 12.14-12.59 (m, |
| | 1 H) |
| C400 | 1H NMR (400 MHz, CHLOROFORM-D δ ppm 1.05-1.29 (m, 10 H) 2.43-2.51 (m, 1 H) 2.62 |
| | (br s, 1 H) 3.75 (br d, J = 7.7 Hz, 1 H) 4.09 (br d, J = |
| | 59.0 Hz, 2 H) 6.83 (t, J = 6.5 Hz, 1 H) 7.27-7.45 (m, 4 H) 7.50-7.54 (m, |
| | 2 H) 7.73 (ddd, J = 9.1, 7.2, 2.0 Hz, 1 H) 8.14-8.25 (m, 3 H) |
| C347a | 1H NMR (400 MHz, DMSO-d₆) δ ppm 1.04 (d, J = 6.2 Hz, 2 H) 1.18 (d, J = |
| | 6.8 Hz, 3 H) 1.34 (d, J = 7.0 Hz, 3 H) 2.31 (br d, J = 16.3 Hz, 1 H) 2.51- |
| | 2.57 (m, 1 H) 2.83 (d, J = 4.6 Hz, 3 H) 3.71 (s, 3 H) 4.00 (br d, J = 19.1 Hz, |
| | 1 H) 4.17-4.77 (m, 2 H) 4.43 (s, 1 H) 5.16 (br t, J = 7.2 Hz, 1 H) 5.57 (d, |
| | J = 7.9 Hz, 1 H) 6.81 (m, J = 8.6 Hz, 2 H) 7.16 (m, J = 8.6 Hz, 2 H) 7.34 (br |
| | s, 1 H) 7.39 (dd, J = 8.1, 2.0 Hz, 1 H) 7.65 (d, J = 2.0 Hz, 1 H) 7.68 (d, J = |
| | 8.4 Hz, 1 H) 7.99 (d, J = 7.9 Hz, 2 H) 8.28-8.38 (m, 1 H) |
| C347b | 1H NMR (400 MHz, DMSO-d₆) δ ppm 1.14 (d, J = 6.8 Hz, 3 H) 1.33 (d, J = |
| | 6.8 Hz, 3 H) 2.27-2.35 (m, 1 H) 2.50-2.57 (m, 1 H) 2.82 (d, J = 4.6 Hz, 3 |
| | H) 3.71 (s, 3 H) 3.96 (br d, J = 18.3 Hz, 1 H) 4.50 (br dd, J = 11.1, 4.1 Hz, |
| | 1 H) 5.14-5.23 (m, 1 H) 5.56 (d, J = 7.9 Hz, 1 H) 6.82 (d, J = 7.8 Hz, 2 H) |
| | 7.17 (d, J = 8.6 Hz, 2 H) 7.31-7.44 (m, 3 H) 7.68 (d, J = 1.8 Hz, 1 H) 7.70 |
| | (d, J = 8.1 Hz, 1 H) 7.98 (d, J = 8.6 Hz, 2 H) 8.29-8.37 (m, 1 H) |
| C401b | 1H NMR (400 MHz, DMSO-d₆) δ ppm 0.99-1.04 (m, 4 H) 1.52-1.62 (m, |
| | 1 H) 1.62-1.74 (m, 2 H) 2.13-2.22 (m, 1 H) 2.22-2.30 (m, 1 H) 2.50- |
| | 2.60 (m, 2 H) 2.83 (d, J = 4.6 Hz, 3 H) 3.21 (ddd, J = 10.2, 6.7, 4.0 Hz, 1 |
| | H) 3.72-3.83 (m, 1 H) 4.33-4.61 (m, 2 H) 5.01 (t, J = 7.6 Hz, 1 H) 7.15-7.24 (m, 3 H) |
| | 7.24-7.36 (m, 3 H) 7.40 (dd, J = 8.1, 2.0 Hz, 1 H) 7.66 (d, |
| | J = 2.0 Hz, 1 H) 7.70 (d, J = 8.4 Hz, 1 H) 7.94 (br d, J = 8.6 Hz, 2 H) 8.28-8.36 (m, 1 H) |
| C401a | 1H NMR (400 MHz, DMSO-d₆) δ ppm 1.16 (d, J = 6.8 Hz, 3 H) 1.53-1.76 |
| | (m, 2 H) 1.69-1.78 (m, 1 H) 2.15-2.23 (m, 1 H) 2.23-2.32 (m, 1 H) |
| | 2.40-2.46 (m, 2 H) 2.51-2.57 (m, 1 H) 2.83 (d, J = 4.6 Hz, 3 H) 3.16-3.26 (m, 1 H) |
| | 3.91-4.01 (m, 1 H) 4.20-4.48 (m, 1 H) 5.00 (br t, J = 7.2 |
| | Hz, 1 H) 7.16-7.41 (m, 8 H) 7.61 (d, J = 1.8 Hz, 1 H) 7.67 (d, J = 8.4 Hz, 1 |
| | H) 7.96 (d, J = 8.6 Hz, 2 H) 8.32 (br d, J = 4.6 Hz, 1 H) |
| C402a | 1H NMR (400 MHz, DMSO-d₆) δ ppm 1.15 (d, J = 6.8 Hz, 3 H) 1.29 (d, J = |
| | 6.8 Hz, 3 H) 2.29-2.37 (m, 1 H) 2.51-2.59 (m, 1 H) 2.84 (d, J = 4.6 Hz, 3 |
| | H) 3.49 (s, 3 H) 3.93-4.08 (m, 1 H) 4.37-4.65 (m, 1 H) 5.26 (quin, J = |
| | 7.4 Hz, 1 H) 5.67-5.73 (m, 1 H) 6.85 (td, J = 7.4, 1.0 Hz, 1 H) 6.91 (d, J = |
| | 7.7 Hz, 1 H) 7.13 (dd, J = 7.5, 1.5 Hz, 1 H) 7.18 (ddd, J = 8.2, 7.4, 1.8 Hz, |
| | 1 H) 7.31-7.50 (m, 3 H) 7.69 (d, J = 2.0 Hz, 1 H) 7.71 (d, J = 8.1 Hz, 1 H) |
| | 8.03 (br s, 2 H) 8.37 (br d, J = 4.2 Hz, 1 H) |
| C402b | 1H NMR (400 MHz, DMSO-d₆) δ ppm 1.19 (d, J = 6.8 Hz, 3 H) 1.29 (d, J = |
| | 6.8 Hz, 3 H) 2.32 (br d, J = 16.5 Hz, 1 H) 2.51-2.57 (m, 1 H) 2.84 (d, J = |
| | 4.4 Hz, 3 H) 3.51 (s, 3 H) 4.03 (br d, J = 19.4 Hz, 1 H) 4.45 (br s, 2 H) |
| | 5.22-5.33 (m, 1 H) 5.66 (d, J = 8.6 Hz, 1 H) 6.82-6.87 (m, 1 H) 6.90 (d, |
| | J = 8.1 Hz, 1 H) 7.13 (d, J = 7.1 Hz, 1 H) 7.17 (t, J = 8.0 Hz, 1 H) 7.23- |
| | 7.54 (m, 1 H) 7.39 (dd, J = 8.1, 2.0 Hz, 1 H) 7.66 (d, J = 2.0 Hz, 1 H) 7.69 |
| | (d, J = 8.4 Hz, 1 H) 8.03 (br d, J = 7.3 Hz, 2 H) 8.37 (br d, J = 4.6 Hz, 1 H) |
| C403a | 1H NMR (400 MHz, DMSO-d₆, 126° C.) δ ppm 1.20 (d, J = 6.2 Hz, 6 H) |
| | 1.23 (d, J = 6.8 Hz, 3 H) 2.44 (d, J = 16.7 Hz, 1 H) 2.67 (br dd, J = 16.6, 6.4 |
| | Hz, 1 H) 2.86 (d, J = 4.6 Hz, 3 H) 4.12 (br d, J = 19.1 Hz, 1 H) 4.49-4.61 |
| | (m, 1 H) 4.69 (br d, J = 19.2 Hz, 1 H) 5.26 (dt, J = 12.4, 6.2 Hz, 1 H) 7.45 |
| | (dd, J = 8.3, 2.0 Hz, 1 H) 7.51 (d, J = 8.2 Hz, 1 H) 7.68 (s, 1 H) 7.70 (d, J = |
| | 6.8 Hz, 1 H) 7.89 (dd, J = 8.2, 1.9 Hz, 1 H) 8.03 (d, J = 1.9 Hz, 1 H) 8.21 |
| | (br s, 1 H) |
| C403b | 1H NMR (400 MHz, DMSO-d₆, 126° C.) δ ppm 1.15-1.26 (m, 9 H) 2.39-2.48 (m, 1 H) 2.59-2.70 |
| | (m, 1 H) 2.83-2.89 (m, 3 H) 4.15 (br d, J = 19.1 |
| | Hz, 1 H) 4.52-4.61 (m, 1 H) 4.66 (br d, J = 19.1 Hz, 1 H) 5.26 (quin, J = |
| | 6.2 Hz, 1 H) 7.39-7.45 (m, 1 H) 7.48 (d, J = 8.2 Hz, 1 H) 7.64-7.68 (m, 1 |
| | H) 7.68-7.73 (m, 1 H) 7.84-7.92 (m, 1 H) 8.00-8.04 (m, 1 H) 8.20 (s, 1 |
| | H) |
| C393a | 1H NMR (400 MHz, DMSO-d₆) δ ppm 1.03-1.10 (m, 6 H) 1.15-1.22 (m, |
| | 3 H) 2.28-2.38 (m, 1 H) 2.52-2.62 (m, 1 H) 3.94-4.08 (m, 1 H) 4.14-4.26 (m, 1 H) |
| | 4.30-4.76 (m, 2 H) 5.47-5.58 (m, 1 H) 7.07-7.30 (m, 2 |
| | H) 7.45 (s, 2 H) 7.67-7.74 (m, 1 H) 7.70 (s, 1 H) 7.99-8.06 (m, 1 H) |
| | 8.16-8.21 (m, 1 H) 12.21-12.79 (m, 1 H) |
| C393b | 1H NMR (400 MHz, DMSO-d₆) δ ppm 1.03-1.10 (m, 6 H) 1.14-1.21 (m, |
| | 3H) 1.17 (s, 1 H) 2.29-2.40 (m, 1 H) 2.50-2.58 (m, 1 H) 2.53 (s, 1 H) |
| | 3.99-4.11 (m, 1 H) 4.13-4.27 (m, 1 H) 4.31-4.76 (m, 2 H) 5.45-5.61 |
| | (m, 1 H) 5.53 (s, 1 H) 7.09-7.31 (m, 2 H) 7.39-7.46 (m, 2 H) 7.67-7.69 |
| | (m, 1 H) 7.69-7.73 (m, 1 H) 8.00-8.07 (m, 1 H) 8.15-8.20 (m, 1 H) |
| | 12.30-12.84 (m, 1 H) |
| C404 | 1H NMR (400 MHz, DMSO-d₆) δ ppm 1.09 (dd, J = 6.5, 3.0 Hz, 6 H) 1.20 |
| | (d, J = 6.8 Hz, 3 H) 2.33-2.40 (m, 1 H) 2.49-2.52 (m, 2 H) 2.53-2.61 |
| | (m, 1 H) 3.98-4.14 (m, 1 H) 4.15-4.31 (m, 1 H) 4.35-4.82 (m, 2 H) |
| | 5.64 (d, J = 8.1 Hz, 1 H) 6.61 (dd, J = 2.4, 1.8 Hz, 1 H) 7.41-7.46 (m, 1 H) |
| | 7.50 (d, J = 8.6 Hz, 1 H) 7.70 (d, J = 1.8 Hz, 1 H) 7.72 (d, J = 8.1 Hz, 1 H) |
| | 7.82 (d, J = 1.8 Hz, 1 H) 7.99 (dd, J = 8.6, 2.4 Hz, 1 H) 8.16 (d, J = 2.4 Hz, |
| | 1 H) 8.58-8.63 (m, 1 H) |
| C399a | 1H NMR (400 MHz, DMSO-d₆) δ ppm 1.05 (dd, J = 6.5, 2.1 Hz, 6 H) 1.19 |
| | (d, J = 6.8 Hz, 3 H) 2.04 (s, 3 H) 2.31-2.40 (m, 1 H) 2.53-2.61 (m, 1 H) |
| | 3.92-4.09 (m, 1 H) 4.12-4.23 (m, 1 H) 4.25-4.76 (m, 2 H) 4.52 (s, 1 H) |
| | 5.08 (d, J = 7.7 Hz, 1 H) 6.99-7.18 (m, 1 H) 7.12 (s, 1 H) 7.21 (d, J = 8.1 |
| | Hz, 1 H) 7.44 (dd, J = 8.4, 2.0 Hz, 1 H) 7.68-7.72 (m, 2 H) 7.89 (dd, J = |
| | 8.3, 1.7 Hz, 1 H) 7.95-8.01 (m, 1 H) 12.14-12.66 (m, 1 H) |
| C399b | 1H NMR (400 MHz, DMSO-d₆) δ ppm 1.05 (dd, J = 6.5, 1.9 Hz, 6 H) 1.18 |
| | (d, J = 6.8 Hz, 3 H) 2.04 (s, 3 H) 2.32-2.40 (m, 1 H) 2.51-2.59 (m, 2 H) |
| | 3.99-4.10 (m, 1 H) 4.10-4.22 (m, 1 H) 4.37-4.66 (m, 1 H) 5.10 (d, J = |
| | 7.9 Hz, 1 H) 7.03-7.15 (m, 1 H) 7.15-7.27 (m, 2 H) 7.21 (s, 1 H) 7.42 |
| | (dd, J = 8.3, 1.9 Hz, 1 H) 7.69 (d, J = 1.8 Hz, 1 H) 7.71 (d, J = 8.1 Hz, 1 H) |
| | 7.89 (dd, J = 8.1, 1.8 Hz, 1 H) 7.97-8.00 (m, 1 H) 12.40 (br d, J = 4.6 Hz, |
| | 1 H) |
| C405 | 1H NMR (400 MHz, DMSO-d₆, 100° C.) δ ppm 1.19 (d, J = 6.8 Hz, 3 H) |
| | 2.36 (d, J = 16.2 Hz, 1 H) 2.50-2.60 (m, 1 H) 2.82 (d, J = 4.6 Hz, 3 H) |
| | 3.19 (s, 3 H) 3.67 (s, 3 H) 3.98 (br d, J = 18.3 Hz, 1 H) 4.52 (s, 2 H) 6.76 |
| | (d, J = 3.2 Hz, 1 H) 7.16-7.22 (m, 2 H) 7.41 (dd, J = 8.2, 1.9 Hz, 1 H) 7.66 |
| | (d, J = 2.0 Hz, 1 H) 7.68 (d, J = 8.1 Hz, 1 H) 7.75 (d, J = 3.1 Hz, 1 H) 7.82-7.88 |
| | (m, 2 H) 8.11 (br d, J = 5.2 Hz, 1 H) |
| C406b | 1H NMR (400 MHz, DMSO-d₆, 81° C.) δ ppm 0.94 (d, J = 6.5 Hz, 3 H) |
| | 1.19 (d, J = 6.8 Hz, 3 H) 1.52-1.96 (m, 6 H) 2.17-2.40 (m, 2 H) 2.49-2.59 (m, 1 H) |
| | 2.83 (d, J = 4.5 Hz, 3 H) 3.87-4.13 (m, 2 H) 4.14-4.79 (m, |
| | 2 H) 4.84 (br d, J = 8.2 Hz, 1 H) 7.32 (br d, J = 7.9 Hz, 2 H) 7.42 (dd, J = |
| | 8.4, 2.0 Hz, 1 H) 7.68 (d, J = 1.8 Hz, 1 H) 7.70 (d, J = 8.1 Hz, 1 H) 7.98 (d, |
| | J = 8.4 Hz, 2 H) 8.28-8.39 (m, 1 H) |
| C406a | 1H NMR (400 MHz, DMSO-d₆, 81° C.) δ ppm 0.94 (d, J = 6.5 Hz, 3 H) |
| | 1.18 (d, J = 6.8 Hz, 3 H) 1.53-1.96 (m, 6 H) 2.20-2.39 (m, 2 H) 2.50-2.58 (m, 1 H) |
| | 2.83 (d, J = 4.6 Hz, 3 H) 3.89-4.12 (m, 2 H) 4.22-4.74 (m, |
| | 2 H) 4.81 (d, J = 8.1 Hz, 1 H) 7.32 (d, J = 8.4 Hz, 2 H) 7.42 (dd, J = 8.2, 2.0 |
| | Hz, 1 H) 7.68 (d, J = 2.0 Hz, 1 H) 7.71 (d, J = 8.1 Hz, 1 H) 7.98 (d, J = 8.4 |
| | Hz, 2 H) 8.28-8.38 (m, 1 H) |
| C407 | 1H NMR (400 MHz, DMSO-d₆) δ ppm 1.09 (d, J = 6.8 Hz, 3 H) 1.51-1.68 |
| | (m, 6 H) 2.27 (d, J = 16.5 Hz, 1 H) 2.51-2.57 (m, 1 H) 2.83 (d, J = 4.6 Hz, |
| | 3 H) 3.71 (br d, J = 18.7 Hz, 1 H) 4.29 (br d, J = 86.7 Hz, 2 H) 4.84 (s, 1 H) |
| | 7.10-7.15 (m, 1 H) 7.22 (br t, J = 7.6 Hz, 2 H) 7.26-7.31 (m, 2 H) 7.33 |
| | (dd, J = 8.3, 1.9 Hz, 1 H) 7.47 (m, J = 8.6 Hz, 2 H) 7.61 (d, J = 2.0 Hz, 1 H) |
| | 7.68 (d, J = 8.1 Hz, 1 H) 8.03 (m, J = 8.6 Hz, 2 H) 8.36 (br d, J = 4.4 Hz, 1 |
| | H) |
| C408 | 1H NMR (400 MHz, DMSO-d₆) δ ppm 1.14-1.17 (m, 2 H) 1.18 (d, J = 6.8 |
| | Hz, 3 H) 1.77 (br s, 2 H) 2.31-2.39 (m, 1 H) 2.51-2.55 (m, 2 H) 2.54- |
| | 2.59 (m, 1 H) 2.60 (br dd, J = 6.6, 3.3 Hz, 1 H) 2.82 (d, J = 4.6 Hz, 3 H) |
| | 4.04 (br d, J = 18.7 Hz, 1 H) 4.39 (br d, J = 7.0 Hz, 1 H) 4.43-4.64 (m, 1 |
| | H) 7.39-7.43 (m, 1 H) 7.44 (d, J = 8.1 Hz, 1 H) 7.68 (d, J = 1.8 Hz, 1 H) |
| | 7.70 (d, J = 8.4 Hz, 1 H) 7.92 (d, J = 8.8 Hz, 2 H) 8.30 (q, J = 4.3 Hz, 1 H) |
| C409 | 1H NMR (400 MHz, DMSO-d₆) δ ppm 1.05 (dd, J = 6.5, 3.2 Hz, 6 H) 1.18 |
| | (d, J = 6.6 Hz, 3 H) 2.07 (s, 3 H) 2.32 (d, J = 16.5 Hz, 1 H) 2.52 (br s, 1 H) |
| | 4.00 (br d, J = 18.0 Hz, 1 H) 4.05-4.15 (m, 1 H) 4.38-4.57 (m, 2 H) 4.78 |
| | (d, J = 7.9 Hz, 1 H) 7.13 (d, J = 8.6 Hz, 2 H) 7.40 (dd, J = 8.1, 2.0 Hz, 1 H) |
| | 7.62-7.76 (m, 4 H) 9.84 (s, 1 H) |
| C410 | 1H NMR (400 MHz, DMSO-d₆) δ ppm 1.05 (dd, J = 6.49, 3.19 Hz, 6 H) |
| | 1.18 (d, J = 6.60 Hz, 3 H) 2.07 (s, 3 H) 2.32 (d, J = 16.51 Hz, 1 H) 2.52 (br |
| | s, 1 H) 4.00 (br d, J = 18.05 Hz, 1 H) 4.05-4.15 (m, 1 H) 4.38-4.57 (m, 2 |
| | H) 4.78 (d, J = 7.92 Hz, 1 H) 7.13 (d, J = 8.58 Hz, 2 H) 7.40 (dd, J = 8.14, |
| | 1.98 Hz, 1 H) 7.62-7.76 (m, 4 H) 9.84 (s, 1 H) |
| C411a | 1H NMR (400 MHz, DMSO-d₆, 81° C.) δ ppm 1.13 (d, J = 6.8 Hz, 3 H) |
| | 1.35 (d, J = 7.0 Hz, 3 H) 2.31 (d, J = 16.4 Hz, 1 H) 2.50-2.59 (m, 1 H) |
| | 2.83 (d, J = 4.5 Hz, 3 H) 3.93 (br d, J = 18.6 Hz, 1 H) 4.29-4.72 (m, 2 H) |
| | 5.25 (quin, J = 7.2 Hz, 1 H) 5.76 (d, J = 8.1 Hz, 1 H) 6.88-7.27 (m, 3 H) |
| | 7.32 (d, J = 8.5 Hz, 2 H) 7.37 (d, J = 8.4 Hz, 2 H) 7.41 (dd, J = 8.2, 2.0 Hz, |
| | 1 H) 7.67 (d, J = 2.0 Hz, 1 H) 7.70 (d, J = 8.1 Hz, 1 H) 7.99 (d, J = 8.4 Hz, 2 |
| | H) 8.28-8.39 (m, 1 H) |
| C412 | 1H NMR (400 MHz, DMSO-d₆) δ ppm 1.11-1.21 (m, 3 H) 1.37-1.44 (m, |
| | 3 H) 2.34 (s, 1 H) 2.54-2.62 (m, 1 H) 2.85 (d, J = 4.6 Hz, 3 H) 3.15-3.16 |
| | (m, 3 H) 3.95 (br dd, J = 24.3, 17.7 Hz, 1 H) 4.21-4.75 (m, 2 H) 5.27- |
| | 5.41 (m, 1 H) 5.97-6.06 (m, 1 H) 7.35-7.52 (m, 3 H) 7.52-7.60 (m, 2 |
| | H) 7.64-7.76 (m, 2 H) 7.82-7.89 (m, 2 H) 8.00-8.06 (m, 2 H) 8.35 (br |
| | d, J = 4.4 Hz, 1 H) |
| C413 | 1H NMR (400 MHz, DMSO-d₆) δ ppm 1.10-1.22 (m, 3 H) 1.37-1.43 (m, |
| | 3 H) 2.28-2.37 (m, 1 H) 2.53-2.59 (m, 1 H) 2.85 (d, J = 4.4 Hz, 3 H) |
| | 3.86-4.07 (m, 1 H) 4.27-4.62 (m, 2 H) 5.24-5.38 (m, 1 H) 5.98 (br d, |
| | J = 7.7 Hz, 1 H) 7.36-7.46 (m, 3 H) 7.48-7.55 (m, 2 H) 7.62 (s, 3 H) |
| | 7.66-7.74 (m, 2 H) 8.02 (d, J = 8.6 Hz, 2 H) 8.32-8.40 (m, 1 H) |
| C414b | 1H NMR (400 MHz, DMSO-d₆, 81° C.) δ ppm 1.18 (d, J = 6.8 Hz, 3 H) |
| | 1.32 (d, J = 7.0 Hz, 3 H) 2.30 (d, J = 16.3 Hz, 1 H) 2.48-2.56 (m, 1 H) |
| | 2.83 (d, J = 4.6 Hz, 3 H) 4.00 (br d, J = 18.7 Hz, 1 H) 4.18-4.69 (m, 2 H) |
| | 5.13 (quin, J = 7.3 Hz, 1 H) 5.62 (d, J = 7.8 Hz, 1 H) 5.92 (s, 2 H) 6.68- |
| | 6.78 (m, 2 H) 6.79-6.84 (m, 1 H) 7.26-7.46 (m, 3 H) 7.65 (d, J = 1.9 Hz, |
| | 1 H) 7.69 (d, J = 8.2 Hz, 1 H) 7.99 (d, J = 8.6 Hz, 2 H) 8.27-8.39 (m, 1 H) |
| C415a | 1H NMR (400 MHz, DMSO-d₆, 81° C.) δ ppm 1.14 (d, J = 6.8 Hz, 3 H) |
| | 1.34 (d, J = 6.9 Hz, 3 H) 2.32 (br d, J = 16.4 Hz, 1 H) 2.50-2.59 (m, 1 H) |
| | 2.83 (d, J = 4.5 Hz, 3 H) 3.71 (s, 3 H) 3.72 (s, 3 H) 3.97 (br d, J = 18.8 Hz, |
| | 1 H) 4.25-4.83 (m, 2 H) 5.17 (quin, J = 7.2 Hz, 1 H) 5.55 (d, J = 8.0 Hz, 1 |
| | H) 6.77 (br dd, J = 8.4, 1.8 Hz, 1 H) 6.84 (d, J = 8.4 Hz, 1 H) 6.86 (br d, J = |
| | 1.8 Hz, 1 H) 7.37 (d, J = 8.2 Hz, 2 H) 7.42 (dd, J = 8.2, 2.0 Hz, 1 H) 7.68 |
| | (d, J = 1.9 Hz, 1 H) 7.71 (d, J = 8.2 Hz, 1 H) 7.99 (d, J = 8.1 Hz, 2 H) 8.28- |
| | 8.38 (m, 1 H) |
| C416b | 1H NMR (400 MHz, DMSO-d₆, 81° C.) δ ppm 1.18 (d, J = 6.8 Hz, 3 H) |
| | 1.33 (d, J = 7.0 Hz, 3 H) 2.31 (d, J = 16.5 Hz, 1 H) 2.46-2.56 (m, 1 H) |
| | 2.83 (d, J = 4.6 Hz, 3 H) 3.79 (s, 3H) 3.99 (br d, J = 17.2 Hz, 1 H) 4.17- |
| | 4.75 (m, 2 H) 5.16 (quin, J = 7.1 Hz, 1 H) 5.74 (d, J = 7.7 Hz, 1 H) 6.97- |
| | 7.10 (m, 3 H) 7.27-7.46 (m, 3 H) 7.65 (d, J = 1.9 Hz, 1 H) 7.68 (d, J = 8.2 |
| | Hz, 1 H) 7.99 (d, J = 8.9 Hz, 2 H) 8.29-8.39 (m, 1 H) |
| C415b | 1H NMR (400 MHz, DMSO-d₆, 81° C.) δ ppm 1.19 (d, J = 6.8 Hz, 3 H) |
| | 1.34 (d, J = 7.0 Hz, 3 H) 2.31 (br d, J = 16.4 Hz, 1 H) 2.48-2.56 (m, 1 H) |
| | 2.83 (d, J = 4.6 Hz, 3 H) 3.69 (s, 3 H) 3.71 (s, 3 H) 4.03 (br d, J = 18.5 Hz, |
| | 1 H) 4.22-4.82 (m, 2 H) 5.13 (quin, J = 7.2 Hz, 1 H) 5.60 (d, J = 7.9 Hz, 1 |
| | H) 6.73-6.79 (m, 1 H) 6.79-6.88 (m, 2 H) 7.27-7.47 (m, 3 H) 7.66 (d, |
| | J = 1.9 Hz, 1 H) 7.69 (d, J = 8.2 Hz, 1 H) 7.99 (d, J = 8.3 Hz, 2 H) 8.29-8.38 (m, 1 H) |
| C416a | 1H NMR (400 MHz, DMSO-d₆, 81° C.) δ ppm 1.14 (d, J = 6.8 Hz, 3 H) |
| | 1.33 (d, J = 7.0 Hz, 3 H) 2.31 (br d, J = 16.4 Hz, 1 H) 2.50-2.58 (m, 1 H) |
| | 2.83 (d, J = 4.4 Hz, 3 H) 3.80 (s, 3 H) 3.95 (br d, J = 19.3 Hz, 1 H) 4.23- |
| | 4.81 (m, 2 H) 5.19 (quin, J = 7.2 Hz, 1 H) 5.72 (d, J = 8.0 Hz, 1 H) 6.99-7.15 (m, 3 H) |
| | 7.36 (d, J = 8.4 Hz, 2 H) 7.41 (dd, J = 8.2, 2.0 Hz, 1 H) 7.68 |
| | (d, J = 1.9 Hz, 1 H) 7.70 (d, J = 8.2 Hz, 1 H) 7.99 (d, J = 8.3 Hz, 2 H) 8.29- |
| | 8.38 (m, 1 H) |
| C411b | 1H NMR (400 MHz, DMSO-d₆, 81° C.) δ ppm 1.17 (d, J = 6.8 Hz, 3 H) |
| | 1.35 (d, J = 7.0 Hz, 3 H) 2.31 (br d, J = 16.3 Hz, 1 H) 2.48-2.55 (m, 1 H) |
| | 2.83 (d, J = 4.4 Hz, 3 H) 3.98 (br d, J = 18.9 Hz, 1 H) 4.15-4.74 (m, 2 H) |
| | 5.22 (quin, J = 7.2 Hz, 1 H) 5.78 (d, J = 7.8 Hz, 1 H) 6.83-7.49 (m, 8 H) |
| | 7.65 (d, J = 1.9 Hz, 1 H) 7.68 (d, J = 8.2 Hz, 1 H) 7.99 (d, J = 8.4 Hz, 2 H) |
| | 8.29-8.38 (m, 1 H) |
| C417b | 1H NMR (400 MHz, DMSO-d₆, 81° C.) δ ppm 1.17 (d, J = 6.8 Hz, 3 H) |
| | 1.34 (d, J = 7.0 Hz, 3 H) 2.26-2.35 (m, 1 H) 2.48-2.56 (m, 1 H) 2.83 (d, |
| | J = 4.5 Hz, 3 H) 3.98 (br d, J = 18.7 Hz, 1 H) 4.16-4.79 (m, 2 H) 5.13- |
| | 5.25 (m, 1 H) 5.88 (br d, J = 7.7 Hz, 1 H) 7.06-7.44 (m, 6 H) 7.64 (d, J = |
| | 1.9 Hz, 1 H) 7.68 (d, J = 8.3 Hz, 1 H) 8.00 (d, J = 8.2 Hz, 2 H) 8.28-8.39 |
| | (m, 1 H) |
| C417a | 1H NMR (400 MHz, DMSO-d₆, 81° C.) δ ppm 1.13 (d, J = 6.8 Hz, 3 H) |
| | 1.34 (d, J = 7.0 Hz, 3 H) 2.30 (d, J = 16.4 Hz, 1 H) 2.49-2.58 (m, 1 H) |
| | 2.83 (d, J = 4.6 Hz, 3 H) 3.93 (br d, J = 18.8 Hz, 1 H) 4.23-4.84 (m, 2 H) |
| | 5.23 (quin, J = 7.2 Hz, 1 H) 5.87 (d, J = 8.0 Hz, 1 H) 7.08-7.16 (m, 1 H) |
| | 7.21-7.34 (m, 2 H) 7.38 (d, J = 8.1 Hz, 2 H) 7.41 (dd, J = 8.4, 2.0 Hz, 1 H) |
| | 7.67 (d, J = 1.9 Hz, 1 H) 7.70 (d, J = 8.2 Hz, 1 H) 8.00 (d, J = 8.5 Hz, 2 H) |
| | 8.28-8.39 (m, 1 H) |
| C414a | 1H NMR (400 MHz, DMSO-d₆, 81° C.) δ ppm 1.14 (d, J = 6.8 Hz, 3 H) |
| | 1.32 (d, J = 7.0 Hz, 3 H) 2.31 (br d, J = 16.4 Hz, 1 H) 2.50-2.58 (m, 1 H) |
| | 2.83 (d, J = 4.6 Hz, 3 H) 3.96 (br d, J = 18.8 Hz, 1 H) 4.19-4.84 (m, 2 H) |
| | 5.16 (quin, J = 7.2 Hz, 1 H) 5.61 (d, J = 8.1 Hz, 1 H) 5.89-5.96 (m, 2 H) |
| | 6.71-6.78 (m, 2 H) 6.84 (d, J = 1.6 Hz, 1 H) 7.36 (d, J = 8.2 Hz, 2 H) 7.41 |
| | (dd, J = 8.2, 2.0 Hz, 1 H) 7.68 (d, J = 1.9 Hz, 1 H) 7.70 (d, J = 8.2 Hz, 1 H) |
| | 7.99 (d, J = 8.3 Hz, 2 H) 8.30-8.39 (m, 1 H) |
| C418 | 1H NMR (400 MHz, DMSO-d₆, 101° C.) δ ppm 1.02-1.12 (m, 7 H) 1.22 |
| | (d, J = 6.8 Hz, 3 H) 2.36 (d, J = 16.4 Hz, 1 H) 2.57 (br dd, J = 16.4, 6.1 Hz, |
| | 1 H) 2.81-2.90 (m, 3 H) 4.04 (br d, J = 18.7 Hz, 1 H) 4.15 (dq, J = 13.5, |
| | 6.8 Hz, 1 H) 4.52 (br d, J = 5.0 Hz, 1 H) 4.95 (br d, J = 7.8 Hz, 1 H) 7.19 (t, |
| | J = 54.1 Hz, 1 H) 7.34 (d, J = 8.5 Hz, 1 H) 7.55 (dd, J = 7.7, 1.3 Hz, 1 H) |
| | 7.64 (d, J = 1.5 Hz, 1 H) 7.79 (d, J = 7.9 Hz, 1 H) 8.00 (d, J = 8.6 Hz, 2 H) |
| | 8.25 (br d, J = 5.3 Hz, 1 H) |
| C419 | 1H NMR (400 MHz, DMSO-d₆) δ ppm 1.09 (dd, J = 6.6, 4.2 Hz, 6 H) 1.13- |
| | 1.21 (m, 3 H) 2.27-2.46 (m, 1 H) 2.51-2.65 (m, 1 H) 2.80 (d, J = 4.8 |
| | Hz, 3 H) 3.57-3.63 (m, 3 H) 3.96-4.08 (m, 1 H) 4.16 (dq, J = 13.8, 6.7 |
| | Hz, 1 H) 4.36-4.64 (m, 2 H) 6.10 (br dd, J = 7.7, 3.3 Hz, 1 H) 7.03 (s, 1 |
| | H) 7.07 (s, 1 H) 7.39-7.45 (m, 1 H) 7.68 (s, 1 H) 7.70 (d, J = 8.1 Hz, 1 H) |
| | 8.22-8.30 (m, 1 H) |
| C420 | 1H NMR (400 MHz, DMSO-d₆, 81° C.) δ ppm 0.84-0.94 (m, 1 H) 0.99- |
| | 1.07 (m, 6 H) 1.08-1.13 (m, 3 H) 1.16-1.25 (m, 7 H) 2.35 (br d, J = 16.5 |
| | Hz, 1 H) 2.52-2.64 (m, 2 H) 2.85 (d, J = 4.6 Hz, 3 H) 3.96-4.12 (m, 1 H) |
| | 4.20 (dq, J = 13.6, 6.8 Hz, 1 H) 4.30-4.79 (m, 2 H) 4.98-5.08 (m, 1 H) |
| | 7.16-7.24 (m, 1 H) 7.41-7.49 (m, 1 H) 7.66-7.76 (m, 2 H) 7.81 (dd, J = |
| | 8.1, 2.0 Hz, 1 H) 7.96-8.01 (m, 1 H) 8.34 (br d, J = 5.4 Hz, 1 H) |
| C421 | 1H NMR (400 MHz, DMSO-d₆, 100° C.) δ ppm 1.19 (d, J = 6.8 Hz, 3 H) |
| | 2.38 (br d, J = 16.6 Hz, 1 H) 2.56-2.65 (m, 1 H) 2.84 (d, J = 4.6 Hz, 3 H) |
| | 4.00 (s, 3 H) 4.07 (br d, J = 19.1 Hz, 1 H) 4.43 (s, 1 H) 4.73 (br d, J = 19.3 |
| | Hz, 1 H) 7.39-7.46 (m, 3 H) 7.67 (d, J = 2.0 Hz, 1 H) 7.69 (d, J = 8.2 Hz, 1 |
| | H) 7.91 (br s, 1 H) 7.99 (d, J = 8.1 Hz, 2 H) 8.11 (br d, J = 4.6 Hz, 1 H) |
| | 8.25 (br d, J = 5.4 Hz, 1 H) |
| C422b | 1H NMR (400 MHz, DMSO-d₆, 81° C.) δ ppm 0.63 (d, J = 6.7 Hz, 3 H) 0.78 |
| | (d, J = 6.6 Hz, 3 H) 1.17 (d, J = 6.8 Hz, 3 H) 1.91-2.06 (m, 1 H) 2.24- |
| | 2.34 (m, 1 H) 2.41-2.55 (m, 1 H) 2.84 (d, J = 4.5 Hz, 3 H) 3.72 (s, 3 H) |
| | 3.98 (br d, J = 18.8 Hz, 1 H) 4.15-4.57 (m, 2 H) 4.61 (t, J = 8.5 Hz, 1 H) |
| | 5.31 (d, J = 8.2 Hz, 1 H) 6.80 (d, J = 8.3 Hz, 2 H) 7.10 (d, J = 8.2 Hz, 2 H) |
| | 7.19-7.55 (m, 3 H) 7.64 (d, J = 1.9 Hz, 1 H) 7.68 (d, J = 8.2 Hz, 1 H) 8.03 |
| | (d, J = 8.2 Hz, 2 H) 8.31-8.42 (m, 1 H) |
| C422a | 1H NMR (400 MHz, DMSO-d₆, 81° C.) δ ppm 0.64 (d, J = 6.6 Hz, 3 H) 0.75 |
| | (d, J = 6.6 Hz, 3 H) 1.11 (d, J = 6.8 Hz, 3 H) 1.93-2.04 (m, 1 H) 2.29 (br d, |
| | J = 16.5 Hz, 1 H) 2.49-2.57 (m, 1 H) 2.84 (d, J = 4.5 Hz, 3 H) 3.72 (s, 3 |
| | H) 3.91 (br d, J = 18.6 Hz, 1 H) 4.21-4.67 (m, 2 H) 4.71 (t, J = 8.5 Hz, 1 |
| | H) 5.28 (d, J = 8.5 Hz, 1 H) 6.83 (d, J = 8.5 Hz, 2 H) 7.13 (d, J = 8.4 Hz, 2 |
| | H) 7.37 (br d, J = 8.0 Hz, 2 H) 7.41 (dd, J = 8.2, 2.0 Hz, 1 H) 7.68 (d, J = |
| | 1.9 Hz, 1 H) 7.71 (d, J = 8.2 Hz, 1 H) 8.03 (d, J = 8.2 Hz, 2 H) 8.33-8.42 |
| | (m, 1 H) |
| C423 | 1H NMR (400 MHz, DMSO-d₆, 81° C.) δ ppm 1.18 (d, J = 6.8 Hz, 3 H) |
| | 2.33 (br d, J = 16.4 Hz, 1 H) 2.50-2.59 (m, 1 H) 2.82 (d, J = 4.6 Hz, 3 H) |
| | 4.02 (br d, J = 18.8 Hz, 1 H) 4.19-4.90 (m, 4 H) 6.12-6.38 (m, 1 H) 7.36 |
| | (br d, J = 8.0 Hz, 2 H) 7.41 (dd, J = 8.2, 2.0 Hz, 1 H) 7.59 (s, 1 H) 7.67 (d, |
| | J = 1.9 Hz, 1 H) 7.70 (d, J = 8.2 Hz, 1 H) 7.97 (d, J = 8.8 Hz, 2 H) 8.26- |
| | 8.36 (m, 1 H) |
| C424 | 1H NMR (400 MHz, CHLOROFORM-D δ ppm 1.05-1.18 (m, 6 H) 1.22-1.35 (m, 4 H) 1.36-1.44 |
| | (m, 3 H) 1.47-1.52 (m, 3 H) 2.43-2.56 (m, 1 |
| | H) 2.65 (br s, 1 H) 3.02 (d, J = 5.1 Hz, 3 H) 3.98-4.26 (m, 4 H) 5.15 (br s, |
| | 1 H) 6.74-6.85 (m, 1 H) 6.85-7.00 (m, 1 H) 7.28-7.43 (m, 1 H) 7.49- |
| | 7.58 (m, 2 H) |
| C412a | 1H NMR (400 MHz, DMSO-d₆) δ ppm 1.13-1.22 (m, 3 H) 1.35-1.49 (m, |
| | 3 H) 2.27-2.38 (m, 1 H) 2.52-2.56 (m, 1 H) 2.83-2.89 (m, 3 H) 3.15- |
| | 3.15 (m, 3 H) 3.98 (br d, J = 18.9 Hz, 1 H) 4.22-4.71 (m, 2 H) 5.33 (t, J = |
| | 7.3 Hz, 1 H) 6.02 (d, J = 7.7 Hz, 1 H) 7.39 (br dd, J = 8.1, 2.0 Hz, 3 H) 7.55 |
| | (d, J = 8.4 Hz, 2 H) 7.63-7.72 (m, 2 H) 7.84 (d, J = 8.4 Hz, 2 H) 7.99-8.09 (m, 2 H) |
| | 8.30-8.42 (m, 1 H) |
| C412b | 1H NMR (400 MHz, DMSO-d₆) δ ppm 1.13 (d, J = 6.8 Hz, 3 H) 1.37-1.43 |
| | (m, 3 H) 2.28-2.38 (m, 1 H) 2.53-2.60 (m, 1 H) 2.83-2.89 (m, 3 H) |
| | 3.19-3.22 (m, 3 H) 3.85-4.00 (m, 1 H) 4.27-4.72 (m, 2 H) 5.30-5.41 |
| | (m, 1 H) 5.98-6.05 (m, 1 H) 7.39-7.46 (m, 3 H) 7.57 (d, J = 8.4 Hz, 2 H) |
| | 7.67-7.75 (m, 2 H) 7.82-7.88 (m, 2 H) 8.02 (d, J = 8.6 Hz, 2 H) 8.36 (br |
| | d, J = 4.4 Hz, 1 H) |
| C413a | 1H NMR (400 MHz, DMSO-d₆) δ ppm 1.16-1.22 (m, 3 H) 1.37-1.42 (m, |
| | 3 H) 2.28-2.35 (m, 1 H) 2.54 (br s, 1 H) 2.84-2.88 (m, 3 H) 3.92-4.07 |
| | (m, 1 H) 4.25-4.62 (m, 2 H) 5.24-5.34 (m, 1 H) 5.95-6.03 (m, 1 H) |
| | 7.36-7.45 (m, 3 H) 7.47-7.53 (m, 2 H) 7.58-7.64 (m, 2 H) 7.64-7.72 |
| | (m, 2 H) 8.02 (d, J = 8.8 Hz, 2 H) 8.37 (br s, 1 H) |
| C413b | 1H NMR (400 MHz, DMSO-d₆) δ ppm 1.11-1.16 (m, 3 H) 1.36-1.42 (m, |
| | 3 H) 2.28-2.36 (m, 1 H) 2.53-2.59 (m, 1 H) 2.83-2.87 (m, 3 H) 3.86- |
| | 4.00 (m, 1 H) 4.26-4.76 (m, 2 H) 5.28-5.38 (m, 1 H) 5.98 (d, J = 7.9 Hz, |
| | 1 H) 7.38-7.46 (m, 3 H) 7.52 (d, J = 8.1 Hz, 2 H) 7.63 (d, J = 8.1 Hz, 2 H) |
| | 7.67-7.75 (m, 2 H) 8.02 (d, J = 8.8 Hz, 2 H) 8.33-8.40 (m, 1 H) |
| C425 | 1H NMR (400 MHz, DMSO-d₆, 100° C.) δ ppm 1.06 (dd, J = 6.6, 2.9 Hz, 6 |
| | H) 1.19 (d, J = 6.8 Hz, 3 H) 2.35 (d, J = 16.4 Hz, 1 H) 2.51-2.59 (m, 1 H) |
| | 4.02 (br d, J = 18.7 Hz, 1 H) 4.14 (dq, J = 13.5, 6.8 Hz, 1 H) 4.38-4.64 (m, |
| | 2 H) 4.99 (br d, J = 7.8 Hz, 1 H) 7.32 (d, J = 8.4 Hz, 2 H) 7.41 (dd, J = 8.1, |
| | 2.0 Hz, 1 H) 7.67 (d, J = 2.0 Hz, 1 H) 7.69 (d, J = 8.4 Hz, 1 H) 8.01 (d, J = |
| | 8.2 Hz, 2 H) 8.18-8.42 (m, 1 H) 14.96 (br s, 1 H) |
| C420a | 1H NMR (400 MHz, DMSO-d₆, 101° C.) δ ppm 1.06 (dd, J = 8.6, 6.5 Hz, 6 |
| | H) 1.12 (d, J = 6.8 Hz, 3 H) 1.21 (dd, J = 6.8, 1.7 Hz, 6 H) 2.36 (d, J = 16.4 |
| | Hz, 1 H) 2.53-2.67 (m, 2 H) 2.86 (d, J = 4.6 Hz, 3 H) 4.04 (br d, J = 18.8 |
| | Hz, 1 H) 4.19 (dq, J = 13.6, 6.8 Hz, 1 H) 4.36-4.70 (m, 2 H) 4.86 (br d, J = |
| | 7.9 Hz, 1 H) 7.21 (d, J = 8.2 Hz, 1 H) 7.45 (dd, J = 8.2, 2.0 Hz, 1 H) 7.70 |
| | (s, 2 H) 7.81 (dd, J = 8.2, 2.0 Hz, 1 H) 7.99 (d, J = 2.0 Hz, 1 H) 8.26 (br d, |
| | J = 5.4 Hz, 1 H) |
| C420b | 1H NMR (400 MHz, DMSO-d₆, 150° C.) δ ppm 1.03-1.10 (m, 5 H) 1.11- |
| | 1.17 (m, 3 H) 1.19-1.27 (m, 6 H) 2.35-2.44 (m, 1 H) 2.55-2.62 (m, 1 |
| | H) 2.62-2.72 (m, 1 H) 2.88 (d, J = 4.6 Hz, 3 H) 4.06 (br d, J = 18.7 Hz, 1 |
| | H) 4.12-4.24 (m, 1 H) 4.44-4.64 (m, 3H) 7.19 (d, J = 8.1 Hz, 1 H) 7.42 |
| | (dd, J = 8.2, 1.9 Hz, 1 H) 7.65 (d, J = 1.9 Hz, 1 H) 7.69 (d, J = 8.1 Hz, 1 H) |
| | 7.81 (dd, J = 8.1, 2.0 Hz, 1 H) 7.97 (d, J = 2.0 Hz, 1 H) 8.01-8.11 (m, 1 H) |
| C427 | 1H NMR (400 MHz, DMSO-d₆, 150° C.) δ ppm 1.09 (dd, J = 6.5, 2.7 Hz, 6 |
| | H) 1.23 (d, J = 6.8 Hz, 3 H) 2.35-2.43 (m, 1 H) 2.55-2.64 (m, 1 H) 2.87 |
| | (d, J = 4.6 Hz, 3 H) 4.00-4.08 (m, 1 H) 4.13 (dq, J = 14.0, 6.6 Hz, 1 H) |
| | 4.47-4.62 (m, 2 H) 4.68 (br d, J = 7.7 Hz, 1 H) 7.16 (t, J = 54.3 Hz, 1 H) |
| | 7.34 (d, J = 8.4 Hz, 2 H) 7.60-7.65 (m, 1 H) 7.65-7.70 (m, 1 H) 7.71 (s, |
| | 1 H) 7.99 (d, J = 8.5 Hz, 2 H) 8.03 (s, 1 H) |
| C428 | 1H NMR (400 MHz, DMSO-d₆, 126° C.) δ ppm 1.08 (dd, J = 6.5, 2.9 Hz, 6 |
| | H) 1.23 (d, J = 6.8 Hz, 3 H) 2.37 (d, J = 16.4 Hz, 1 H) 2.54-2.64 (m, 1 H) |
| | 2.81-2.86 (m, 3 H) 4.05 (br d, J = 18.7 Hz, 1 H) 4.14 (dq, J = 14.1, 6.6 Hz, |
| | 1 H) 4.44-4.53 (m, 1 H) 4.57 (br d, J = 19.6 Hz, 1 H) 4.81 (br d, J = 7.7 |
| | Hz, 1 H) 7.34 (d, J = 8.4 Hz, 2 H) 7.72-7.77 (m, 1 H) 7.80 (d, J = 8.4 Hz, 1 |
| | H) 7.85 (d, J = 1.9 Hz, 1 H) 8.00 (d, J = 8.5 Hz, 2 H) 8.14 (br d, J = 5.6 Hz, |
| | 1 H) |
| C429 | 1H NMR (400 MHz, DMSO-d₆, 101° C.) δ ppm 1.07 (dd, J = 6.6, 3.0 Hz, 6 |
| | H) 1.15-1.22 (m, 3 H) 2.30-2.38 (m, 1 H) 2.41 (s, 3 H) 2.51-2.60 (m, 1 |
| | H) 2.85 (d, J = 4.6 Hz, 3 H) 4.01 (br d, J = 18.8 Hz, 1 H) 4.08-4.20 (m, 1 |
| | H) 4.55 (s, 1 H) 4.93 (br d, J = 7.7 Hz, 1 H) 7.26 (dd, J = 8.1, 2.1 Hz, 1 H) |
| | 0.00 (d, J = 8.6 Hz, 2 H) 7.40 (d, J = 2.1 Hz, 1 H) 7.49 (d, J = 8.1 Hz, 1 H) |
| | 0.00 (d, J = 8.8 Hz, 2 H) 8.25 (br d, J = 5.4 Hz, 1 H) |
| C430 | 1H NMR (400 MHz, DMSO-d₆, 100° C.) δ ppm 1.08 (dd, J = 6.6, 3.1 Hz, 6 |
| | H) 1.20 (d, J = 6.8 Hz, 3 H) 2.35 (d, J = 16.3 Hz, 1 H) 2.50-2.60 (m, 1 H) |
| | 4.03 (br d, J = 18.7 Hz, 1 H) 4.17 (dq, J = 13.6, 6.8 Hz, 1 H) 4.51 (s, 2 H) |
| | 5.19 (br d, J = 7.8 Hz, 1 H) 7.41 (dd, J = 8.4, 2.0 Hz, 1 H) 7.45-7.51 (m, 2 |
| | H) 7.66 (d, J = 1.8 Hz, 1 H) 7.69 (d, J = 8.1 Hz, 1 H) 7.93 (d, J = 1.2 Hz, 1 |
| | H) 8.06 (d, J = 8.1 Hz, 2 H) 8.77 (d, J = 1.2 Hz, 1 H) |
| C431 | 1H NMR (400 MHz, DMSO-d₆, 81° C.) δ ppm 1.18 (d, J = 6.8 Hz, 3 H) |
| | 1.33 (d, J = 7.0 Hz, 3 H) 2.24 (s, 3 H) 2.30 (br d, J = 16.4 Hz, 1 H) 2.47- |
| | 2.56 (m, 1 H) 2.83 (d, J = 4.5 Hz, 3 H) 3.99 (br d, J = 18.8 Hz, 1 H) 4.16- |
| | 4.82 (m, 2 H) 5.15 (quin, J = 7.1 Hz, 1 H) 5.60 (d, J = 7.8 Hz, 1 H) 7.00- |
| | 7.08 (m, 2 H) 7.08-7.17 (m, 2 H) 7.25-7.48 (m, 3 H) 7.65 (d, J = 1.9 Hz, |
| | 1 H) 7.68 (d, J = 8.2 Hz, 1 H) 7.99 (d, J = 8.3 Hz, 2 H) 8.28-8.38 (m, 1 H) |
| C432 | 1H NMR (400 MHz, DMSO-d₆, 81° C.) δ ppm 1.16 (d, J = 6.8 Hz, 3 H) |
| | 1.38 (d, J = 7.0 Hz, 3 H) 2.30 (d, J = 16.3 Hz, 1 H) 2.44-2.58 (m, 1 H) |
| | 2.80 (d, J = 4.7 Hz, 3 H) 3.96 (br d, J = 18.9 Hz, 1 H) 4.16-4.70 (m, 2 H) |
| | 5.20 (quin, J = 7.1 Hz, 1 H) 6.40 (br d, J = 7.7 Hz, 1 H) 6.78 (s, 1 H) 6.99- |
| | 7.11 (m, 2 H) 7.31 (dd, J = 8.4, 5.4 Hz, 2 H) 7.38 (dd, J = 8.2, 1.9 Hz, 1 H) |
| | 7.65 (d, J = 1.9 Hz, 1 H) 7.68 (d, J = 8.2 Hz, 1 H) 8.17-8.29 (m, 1 H) |
| | 12.90-14.22 (m, 1 H) |
| C433 | 1H NMR (400 MHz, DMSO-d₆, 81° C.) δ ppm 1.11-1.22 (m, 3 H) 1.33- |
| | 1.45 (m, 3 H) 2.11-2.20 (m, 3 H) 2.33 (br d, J = 16.2 Hz, 1 H) 2.50-2.59 |
| | (m, 1 H) 2.82 (d, J = 4.5 Hz, 3 H) 3.88-4.10 (m, 1 H) 4.18-4.85 (m, 2 H) |
| | 5.34-5.45 (m, 1 H) 6.07 (br d, J = 9.0 Hz, 1 H) 6.13 (br s, 1 H) 7.36 (br d, |
| | J = 8.0 Hz, 2 H) 7.39-7.44 (m, 1 H) 7.65-7.72 (m, 2 H) 7.98 (br d, J = 8.3 |
| | Hz, 2 H) 8.28-8.37 (m, 1 H) |
| C434 | 1H NMR (400 MHz, DMSO-d₆, 81° C.) δ ppm 1.08-1.24 (m, 3 H) 1.51 (d, |
| | J = 6.9 Hz, 3 H) 2.27-2.39 (m, 1 H) 2.51-2.62 (m, 1 H) 2.82 (d, J = 4.5 |
| | Hz, 3 H) 3.89-4.11 (m, 1 H) 4.28-4.76 (m, 2 H) 5.44-5.61 (m, 1 H) |
| | 6.21-6.36 (m, 1 H) 7.26-7.79 (m, 7 H) 7.99 (d, J = 8.2 Hz, 2 H) 8.29- |
| | 8.39 (m, 1 H) |
| C435b | 1H NMR (400 MHz, DMSO-d₆, 81° C.) δ ppm 1.14 (d, J = 6.8 Hz, 3 H) |
| | 1.35 (d, J = 7.0 Hz, 3 H) 2.31 (d, J = 16.4 Hz, 1 H) 2.50-2.58 (m, 1 H) |
| | 2.83 (d, J = 4.6 Hz, 3 H) 3.81 (s, 3 H) 3.95 (br d, J = 18.9 Hz, 1 H) 4.22- |
| | 4.86 (m, 2 H) 5.21 (quin, J = 7.2 Hz, 1 H) 5.74 (d, J = 8.0 Hz, 1 H) 6.82 |
| | (ddd, J = 8.3, 4.3, 2.1 Hz, 1 H) 7.00-7.09 (m, 2 H) 7.38 (d, J = 8.4 Hz, 2 |
| | H) 7.41 (dd, J = 8.2, 2.0 Hz, 1 H) 7.68 (d, J = 1.9 Hz, 1 H) 7.70 (d, J = 8.2 |
| | Hz, 1 H) 7.99 (d, J = 8.3 Hz, 2 H) 8.29-8.38 (m, 1 H) |
| C435a | 1H NMR (400 MHz, DMSO-d₆, 81° C.) δ ppm 1.18 (d, J = 6.8 Hz, 3 H) |
| | 1.35 (d, J = 7.0 Hz, 3 H) 2.27-2.35 (m, 1 H) 2.48-2.55 (m, 1 H) 2.83 (d, |
| | J = 4.6 Hz, 3 H) 3.77 (s, 3 H) 4.01 (br d, J = 18.8 Hz, 1 H) 4.19-4.83 (m, 2 |
| | H) 5.17 (quin, J = 7.2 Hz, 1 H) 5.77 (d, J = 7.9 Hz, 1 H) 6.81 (ddd, J = 8.3, |
| | 4.3, 2.1 Hz, 1 H) 6.98-7.08 (m, 2 H) 7.26-7.48 (m, 3 H) 7.65 (d, J = 1.9 |
| | Hz, 1 H) 7.68 (d, J = 8.2 Hz, 1 H) 7.99 (d, J = 8.3 Hz, 2 H) 8.29-8.38 (m, 1 |
| | H) |
| C436b | 1H NMR (400 MHz, DMSO-d₆, 81° C.) δ ppm 1.11 (d, J = 7.0 Hz, 3 H) |
| | 1.18 (d, J = 6.8 Hz, 3 H) 2.31 (br d, J = 16.3 Hz, 1 H) 2.49-2.58 (m, 1 H) |
| | 2.83 (d, J = 4.6 Hz, 3 H) 2.91-3.07 (m, 1 H) 3.24-3.45 (m, 2 H) 3.70 (s, |
| | 3 H) 4.02 (br d, J = 18.6 Hz, 1 H) 4.17-4.92 (m, 2 H) 5.32 (br t, J = 5.6 Hz, |
| | 1 H) 6.61-6.69 (m, 2 H) 6.70-6.75 (m, 1 H) 7.09-7.20 (m, 3 H) 7.42 |
| | (dd, J = 8.4, 2.0 Hz, 1 H) 7.69 (d, J = 1.9 Hz, 1 H) 7.71 (d, J = 8.2 Hz, 1 H) |
| | 7.92 (d, J = 8.4 Hz, 2 H) 8.25-8.36 (m, 1 H) |
| C436a | 1H NMR (400 MHz, DMSO-d₆, 81° C.) δ ppm 1.12 (d, J = 7.0 Hz, 3 H) |
| | 1.16 (d, J = 6.8 Hz, 3 H) 2.30 (d, J = 16.3 Hz, 1 H) 2.48-2.59 (m, 1 H) |
| | 2.83 (d, J = 4.5 Hz, 3 H) 2.92-3.03 (m, 1 H) 3.24-3.43 (m, 2 H) 3.71 (s, |
| | 3 H) 3.98 (br d, J = 18.7 Hz, 1 H) 4.19-4.93 (m, 2 H) 5.32 (br t, J = 5.6 Hz, |
| | 1 H) 6.63-6.76 (m, 3 H) 7.08-7.25 (m, 3 H) 7.42 (dd, J = 8.1, 2.0 Hz, 1 |
| | H) 7.68 (d, J = 1.9 Hz, 1 H) 7.71 (d, J = 8.2 Hz, 1 H) 7.92 (d, J = 8.2 Hz, 2 |
| | H) 8.26-8.35 (m, 1 H) |
| C437 | 1H NMR (400 MHz, DMSO-d₆, 81° C.) δ ppm −0.20-−0.03 (m, 2 H) 0.15-0.37 (m, 2 H) |
| | 0.41-0.59 (m, 1 H) 1.03-1.24 (m, 3 H) 1.43-1.57 (m, 1 |
| | H) 1.60-1.74 (m, 1 H) 2.24-2.36 (m, 1 H) 2.49-2.58 (m, 1 H) 2.84 (d, |
| | J = 4.6 Hz, 3 H) 3.76-4.06 (m, 1 H) 4.16-4.82 (m, 2 H) 5.02-5.23 (m, 1 |
| | H) 5.57 (br d, J = 7.8 Hz, 1 H) 7.13-7.31 (m, 5 H) 7.33-7.51 (m, 3 H) |
| | 7.60-7.73 (m, 2 H) 8.03 (d, J = 8.2 Hz, 2 H) 8.30-8.42 (m, 1 H) |
| C438 | 1H NMR (400 MHz, DMSO-d₆, 81° C.) δ ppm 1.07-1.21 (m, 3 H) 1.30 (d, |
| | J = 7.0 Hz, 3 H) 2.24-2.35 (m, 1 H) 2.45-2.58 (m, 1 H) 2.84 (d, J = 4.5 |
| | Hz, 3 H) 3.76-4.05 (m, 1 H) 4.09-4.92 (m, 2 H) 5.35-5.53 (m, 1 H) |
| | 5.65-5.80 (m, 1 H) 7.02-7.45 (m, 8 H) 7.59-7.74 (m, 2 H) 7.96-8.07 |
| | (m, 2 H) 8.30-8.41 (m, 1 H) |
| C439b | 1H NMR (400 MHz, DMSO-d₆, 81° C.) δ ppm 1.19 (d, J = 6.8 Hz, 3 H) |
| | 1.42 (d, J = 6.8 Hz, 3 H) 2.34 (br d, J = 16.6 Hz, 1 H) 2.51-2.59 (m, 1 H) |
| | 2.83 (d, J = 4.6 Hz, 3 H) 3.90-4.14 (m, 1 H) 4.22-4.85 (m, 2 H) 5.20-5.38 (m, 1 H) |
| | 5.59-5.83 (m, 1 H) 7.34-7.48 (m, 3 H) 7.67 (d, J = 1.9 Hz, |
| | 1 H) 7.69 (d, J = 8.2 Hz, 1 H) 7.99 (d, J = 8.8 Hz, 2 H) 8.07 (br s, 1 H) 8.29- |
| | 8.38 (m, 1 H) |
| C439a | 1H NMR (400 MHz, DMSO-d₆, 81° C.) δ ppm 1.17 (d, J = 6.8 Hz, 3 H) |
| | 1.42 (d, J = 6.8 Hz, 3 H) 2.34 (br d, J = 16.6 Hz, 1 H) 2.52-2.62 (m, 1 H) |
| | 2.83 (d, J = 4.6 Hz, 3 H) 4.01 (br d, J = 18.6 Hz, 1 H) 4.22-4.86 (m, 2 H) |
| | 5.21-5.34 (m, 1 H) 5.60-5.84 (m, 1 H) 7.35-7.47 (m, 3 H) 7.68 (d, J = |
| | 1.8 Hz, 1 H) 7.71 (d, J = 8.4 Hz, 1 H) 7.99 (d, J = 8.8 Hz, 2 H) 8.08 (br s, 1 |
| | H) 8.30-8.39 (m, 1 H) |
| C434b | 1H NMR (400 MHz, DMSO-d₆, 81° C.) δ ppm 1.18 (d, J = 6.8 Hz, 3 H) |
| | 1.51 (d, J = 7.0 Hz, 3 H) 2.33 (d, J = 16.4 Hz, 1 H) 2.51-2.60 (m, 1 H) |
| | 2.82 (d, J = 4.6 Hz, 3 H) 4.01 (br d, J = 18.9 Hz, 1 H) 4.19-4.87 (m, 2 H) |
| | 5.47-5.59 (m, 1 H) 6.28 (br d, J = 7.2 Hz, 1 H) 7.30-7.47 (m, 3 H) 7.52 |
| | (d, J = 3.3 Hz, 1 H) 7.64 (d, J = 3.3 Hz, 1 H) 7.67 (d, J = 2.0 Hz, 1 H) 7.69 |
| | (d, J = 8.1 Hz, 1 H) 7.99 (d, J = 8.8 Hz, 2 H) 8.30-8.39 (m, 1 H) |
| C434a | 1H NMR (400 MHz, DMSO-d₆, 81° C.) δ ppm 1.16 (d, J = 6.8 Hz, 3 H) |
| | 1.50 (d, J = 6.8 Hz, 3 H) 2.34 (d, J = 16.6 Hz, 1 H) 2.51-2.61 (m, 1 H) |
| | 2.82 (d, J = 4.6 Hz, 3 H) 3.99 (br d, J = 18.7 Hz, 1 H) 4.20-4.96 (m, 2 H) |
| | 5.43-5.58 (m, 1 H) 6.18-6.38 (m, 1 H) 7.38 (d, J = 8.4 Hz, 2 H) 7.41 (dd, |
| | J = 8.3, 1.9 Hz, 1 H) 7.53 (d, J = 3.3 Hz, 1 H) 7.64 (d, J = 3.3 Hz, 1 H) 7.68 |
| | (d, J = 1.8 Hz, 1 H) 7.70 (d, J = 8.1 Hz, 1 H) 7.99 (d, J = 8.6 Hz, 2 H) 8.30-8.38 (m, 1 H) |
| C440b | 1H NMR (400 MHz, DMSO-d₆, 81° C.) δ ppm 1.17 (d, J = 6.8 Hz, 3 H) |
| | 1.36 (d, J = 7.0 Hz, 3 H) 2.25-2.36 (m, 1 H) 2.47-2.57 (m, 1 H) 2.83 (d, |
| | J = 4.4 Hz, 3 H) 3.98 (br d, J = 18.7 Hz, 1 H) 4.15-4.78 (m, 2 H) 5.24 |
| | (quin, J = 7.0 Hz, 1 H) 5.87 (br d, J = 7.3 Hz, 1 H) 7.21 (br d, J = 7.9 Hz, 2 |
| | H) 7.31-7.44 (m, 5 H) 7.64 (d, J = 2.0 Hz, 1 H) 7.67 (d, J = 8.1 Hz, 1 H) |
| | 7.99 (d, J = 8.6 Hz, 2 H) 8.28-8.36 (m, 1 H) |
| C440a | 1H NMR (400 MHz, DMSO-d₆, 81° C.) δ ppm 1.13 (d, J = 6.8 Hz, 3 H) |
| | 1.36 (d, J = 7.0 Hz, 3 H) 2.30 (d, J = 16.3 Hz, 1 H) 2.49-2.58 (m, 1 H) |
| | 2.83 (d, J = 4.4 Hz, 3 H) 3.92 (br d, J = 18.8 Hz, 1 H) 4.14-4.84 (m, 2 H) |
| | 5.22-5.35 (m, 1 H) 5.89 (br d, J = 6.6 Hz, 1 H) 7.19-7.27 (m, 2 H) 7.33- |
| | 7.45 (m, 5 H) 7.67 (d, J = 1.9 Hz, 1 H) 7.70 (d, J = 8.2 Hz, 1 H) 7.99 (d, J = |
| | 8.4 Hz, 2 H) 8.29-8.38 (m, 1 H) |
| C441a | 1H NMR (400 MHz, DMSO-d₆) δ ppm 1.15-1.21 (m, 3 H) 1.34-1.41 (m, |
| | 3 H) 2.27-2.37 (m, 1 H) 2.52-2.57 (m, 1 H) 2.85 (d, J = 4.6 Hz, 3 H) |
| | 3.89-4.06 (m, 1 H) 4.21-4.68 (m, 2 H) 5.27 (quin, J = 7.0 Hz, 1 H) 6.00 |
| | (d, J = 7.5 Hz, 1 H) 7.36-7.43 (m, 3 H) 7.48 (d, J = 8.1 Hz, 2 H) 7.64-7.66 (m, 1 H) |
| | 7.70 (dd, J = 8.4, 4.2 Hz, 3 H) 7.99-8.04 (m, 2 H) 8.35 (br |
| | d, J = 4.4 Hz, 1 H) |
| C441a | 1H NMR (400 MHz, DMSO-d₆) δ ppm 1.11-1.16 (m, 3 H) 1.35-1.40 (m, |
| | 3 H) 2.28-2.35 (m, 1 H) 2.53-2.59 (m, 1 H) 2.81-2.88 (m, 3 H) 3.83-4.00 (m, 1 H) 4.22-4.84 |
| | (m, 2 H) 5.25-5.37 (m, 1 H) 5.97-6.04 (m, 1 |
| | H) 7.38-7.44 (m, 3 H) 7.50 (d, J = 8.1 Hz, 2 H) 7.67-7.69 (m, 1 H) 7.70-7.76 (m, 3 H) |
| | 7.96-8.06 (m, 2 H) 8.36 (br d, J = 4.4 Hz, 1 H) |
| C442 | 1H NMR (400 MHz, DMSO-d₆, 100° C.) δ ppm 1.07 (dd, J = 6.5, 3.2 Hz, 6 |
| | H) 1.20 (d, J = 6.8 Hz, 3 H) 2.35 (d, J = 16.4 Hz, 1 H) 2.50-2.64 (m, 1 H) |
| | 4.03 (br d, J = 18.7 Hz, 1 H) 4.16 (dq, J = 13.5, 6.8 Hz, 1 H) 4.52 (s, 2 H) |
| | 5.26 (br d, J = 7.8 Hz, 1 H) 7.35 (br d, J = 7.7 Hz, 1 H) 7.41 (dd, J = 8.1, 2.0 |
| | Hz, 1 H) 7.66 (d, J = 2.0 Hz, 1 H) 7.69 (d, J = 8.1 Hz, 1 H) 7.75 (t, J = 8.1 |
| | Hz, 1 H) 7.86 (s, 1 H) 7.91 (d, J = 1.1 Hz, 1 H) 8.07 (ddd, J = 8.2, 2.2, 1.0 |
| | Hz, 1 H) 8.73 (d, J = 1.2 Hz, 1 H) |
| C433b | 1H NMR (400 MHz, DMSO-d₆, 81° C.) δ ppm 1.18 (d, J = 6.8 Hz, 3 H) |
| | 1.40 (d, J = 7.0 Hz, 3 H) 2.15 (s, 3 H) 2.33 (d, J = 16.3 Hz, 1 H) 2.50-2.59 |
| | (m, 1 H) 2.83 (d, J = 4.6 Hz, 3 H) 3.99 (br d, J = 18.9 Hz, 1 H) 4.22-4.77 |
| | (m, 2 H) 5.32-5.47 (m, 1 H) 6.06 (s, 1 H) 6.08-6.18 (m, 1 H) 7.36 (br d, |
| | J = 8.1 Hz, 2 H) 7.40 (dd, J = 8.2, 2.0 Hz, 1 H) 7.67 (d, J = 1.9 Hz, 1 H) |
| | 7.69 (d, J = 8.2 Hz, 1 H) 7.98 (d, J = 8.8 Hz, 2 H) 8.28-8.36 (m, 1 H) |
| C433a | 1H NMR (400 MHz, DMSO-d₆, 81° C.) δ ppm 1.17 (d, J = 6.8 Hz, 3 H) |
| | 1.39 (d, J = 7.0 Hz, 3 H) 2.16 (s, 3 H) 2.33 (d, J = 16.3 Hz, 1 H) 2.51-2.58 |
| | (m, 1 H) 2.82 (d, J = 4.5 Hz, 3 H) 4.00 (br d, J = 18.9 Hz, 1 H) 4.23-4.80 |
| | (m, 2 H) 5.40 (quin, J = 7.2 Hz, 1 H) 6.08 (s, 1 H) 6.13 (br d, J = 8.0 Hz, 1 |
| | H) 7.36 (br d, J = 7.9 Hz, 2 H) 7.41 (dd, J = 8.2, 2.0 Hz, 1 H) 7.68 (d, J = |
| | 1.9 Hz, 1 H) 7.70 (d, J = 8.2 Hz, 1 H) 7.98 (d, J = 8.8 Hz, 2 H) 8.28-8.36 |
| | (m, 1 H) |
| C443 | 1H NMR (400 MHz, DMSO-d₆, 81° C.) δ ppm 1.15 (d, J = 6.8 Hz, 3 H) |
| | 1.31 (d, J = 7.0 Hz, 3 H) 2.22-2.30 (m, 1 H) 2.39-2.47 (m, 1 H) 2.85 (d, |
| | J = 4.6 Hz, 3 H) 3.88 (br d, J = 18.7 Hz, 1 H) 4.01-4.73 (m, 2 H) 5.37-5.50 (m, 1 H) |
| | 6.04 (br d, J = 6.1 Hz, 1 H) 7.24-7.69 (m, 9 H) 8.03 (d, J = |
| | 8.3 Hz, 2 H) 8.32-8.41 (m, 1 H) |
| C444 | 1H NMR (400 MHz, DMSO-d₆) δ ppm 1.18 (d, J = 6.8 Hz, 3 H) 1.29-1.40 |
| | (m, 2 H) 1.58-1.64 (m, 2 H) 1.67 (s, 3 H) 2.38 (d, J = 16.9 Hz, 1 H) 2.42- |
| | 2.54 (m, 9 H) 2.45-2.52 (m, 1 H) 2.59 (br dd, J = 16.9, 5.9 Hz, 1 H) 2.81 |
| | (d, J = 4.6 Hz, 3 H) 4.05 (br d, J = 18.3 Hz, 1 H) 4.39-4.68 (m, 1 H) 7.39- |
| | 7.45 (m, 2 H) 7.41-7.44 (m, 1 H) 7.68 (d, J = 1.8 Hz, 1 H) 7.70 (d, J = 8.4 |
| | Hz, 1 H) 7.91 (d, J = 8.6 Hz, 2 H) 8.08-8.15 (m, 1 H) 8.28 (br d, J = 4.6 |
| | Hz, 1 H) |
| C445 | 1H NMR (400 MHz, DMSO-d₆, 81° C.) δ ppm 1.16 (d, J = 6.8 Hz, 3 H) |
| | 1.32 (d, J = 7.0 Hz, 3 H) 2.28 (d, J = 16.5 Hz, 1 H) 2.48 (dt, J = 3.8, 1.9 Hz, |
| | 1 H) 2.84 (d, J = 4.6 Hz, 3 H) 3.95 (br d, J = 18.8 Hz, 1 H) 4.09-4.71 (m, 2 |
| | H) 5.40-5.52 (m, 1 H) 5.90 (d, J = 7.2 Hz, 1 H) 7.14-7.48 (m, 7 H) 7.62 |
| | (d, J = 1.9 Hz, 1 H) 7.67 (d, J = 8.2 Hz, 1 H) 8.02 (d, J = 8.6 Hz, 2 H) 8.30-8.40 (m, 1 H) |
| C446 | ¹H NMR (400 MHz, DMSO-d₆) δ ppm 0.62-0.74 (m, 6 H) 1.14-1.30 (m, |
| | 7 H) 1.69 (br dd, J = 11.88, 6.75 Hz, 1 H) 2.30-2.46 (m, 2 H) 2.57-2.69 |
| | (m, 1 H) 2.84 (d, J = 4.63 Hz, 3 H) 4.02-4.23 (m, 1 H) 4.91-5.04 (m, 1 H) |
| | 7.36 (d, J = 8.50 Hz, 2 H) 7.46 (dd, J = 8.19, 1.94 Hz, 1 H) 7.70-7.77 (m, 2 |
| | H) 7.94 (d, J = 8.63 Hz, 2 H) 8.33 (br d, J = 4.25 Hz, 1 H) |
| C447 | 1H NMR (400 MHz, DMSO-d₆, 100° C.) δ ppm 0.99-1.09 (m, 6 H) 1.15- |
| | 1.22 (m, 3 H) 2.03 (s, 3 H) 2.30-2.46 (m, 1 H) 2.50-2.65 (m, 1 H) 2.92 |
| | (d, J = 4.8 Hz, 3 H) 4.03 (br d, J = 18.7 Hz, 1 H) 4.17 (dq, J = 13.8, 6.8 Hz, |
| | 1 H) 4.51 (br s, 2 H) 5.19 (br d, J = 7.9 Hz, 1 H) 6.65 (br d, J = 5.3 Hz, 1 H) |
| | 7.30-7.35 (m, 1 H) 7.38-7.45 (m, 1 H) 7.65-7.72 (m, 2 H) 7.76 (s, 1 H) |
| C448 | 1H NMR (400 MHz, DMSO-d₆, 100° C.) δ ppm 1.06 (dd, J = 6.5, 3.0 Hz, 6 |
| | H) 1.20 (d, J = 6.8 Hz, 3 H) 2.35 (d, J = 16.4 Hz, 1 H) 2.50-2.61 (m, 1 H) |
| | 2.85-3.13 (m, 1 H) 4.03 (br d, J = 18.7 Hz, 1 H) 4.13 (dq, J = 13.6, 6.7 Hz, |
| | 1 H) 4.40-4.63 (m, 2 H) 5.00 (d, J = 7.8 Hz, 1 H) 7.22 (br d, J = 7.8 Hz, 1 |
| | H) 7.41 (dd, J = 8.2, 2.0 Hz, 1 H) 7.62 (t, J = 7.8 Hz, 1 H) 7.67 (d, J = 1.8 |
| | Hz, 1 H) 7.69 (d, J = 8.4 Hz, 1 H) 7.71 (br s, 1 H) 7.98 (d, J = 7.5 Hz, 1 H) |
| | 8.26-8.31 (m, 1 H) |
| C449a | 1H NMR (400 MHz, DMSO-d₆) δ ppm 1.18-1.23 (m, 3 H) 1.40-1.45 (m, |
| | 3 H) 2.30-2.37 (m, 1 H) 2.52-2.58 (m, 1 H) 2.82-2.87 (m, 3 H) 3.98 (s, |
| | 1 H) 4.23-4.74 (m, 2 H) 5.21-5.35 (m, 1 H) 5.46 (d, J = 7.7 Hz, 1 H) |
| | 6.31-6.36 (m, 1 H) 6.96-7.03 (m, 1 H) 7.23-7.35 (m, 3 H) 7.36-7.45 |
| | (m, 3 H) 7.65-7.67 (m, 1 H) 7.68-7.71 (m, 1 H) 7.95-8.07 (m, 2 H) |
| | 8.29-8.42 (m, 1 H) 10.70-10.86 (m, 1 H) |
| C449b | 1H NMR (400 MHz, DMSO-d₆) δ ppm 1.12-1.20 (m, 3 H) 1.39-1.47 (m, |
| | 3 H) 2.29-2.38 (m, 1 H) 2.53-2.61 (m, 1 H) 2.81-2.87 (m, 3 H) 3.91- |
| | 4.09 (m, 1 H) 4.17-4.88 (m, 1 H) 5.25-5.36 (m, 1 H) 5.39-5.47 (m, 1 |
| | H) 6.33-6.40 (m, 1 H) 6.97-7.03 (m, 1 H) 7.25-7.28 (m, 1 H) 7.28-7.33 (m, 1 H) |
| | 7.36-7.46 (m, 4 H) 7.69-7.71 (m, 1 H) 7.71-7.75 (m, 1 |
| | H) 7.96-8.04 (m, 2 H) 8.31-8.39 (m, 1 H) 10.71-10.83 (m, 1 H) |
| C450a | 1H NMR (400 MHz, DMSO-d₆) δ ppm 1.15-1.23 (m, 3 H) 1.39-1.50 (m, |
| | 3 H) 2.27-2.38 (m, 1 H) 2.52-2.57 (m, 1 H) 2.81-2.87 (m, 3 H) 3.69-3.80 (m, 3 H) |
| | 3.97-4.14 (m, 1 H) 4.24-4.68 (m, 2 H) 5.22-5.35 (m, 1 |
| | H) 5.45-5.57 (m, 1 H) 6.29-6.37 (m, 1 H) 7.02-7.11 (m, 1 H) 7.20-7.25 (m, 1 H) |
| | 7.28-7.32 (m, 1 H) 7.33-7.52 (m, 4 H) 7.66-7.67 (m, 1 |
| | H) 7.67-7.71 (m, 1 H) 7.96-8.07 (m, 2 H) 8.30-8.43 (m, 1 H) |
| C450b | 1H NMR (400 MHz, DMSO-d₆) δ ppm 1.10-1.19 (m, 3 H) 1.39-1.47 (m, |
| | 3 H) 2.29-2.37 (m, 1 H) 2.52-2.61 (m, 1 H) 2.82-2.87 (m, 3 H) 3.73-3.78 (m, 3 H) |
| | 3.92-4.12 (m, 1 H) 4.22-4.87 (m, 2 H) 5.26-5.37 (m, 1 |
| | H) 5.46 (d, J = 7.9 Hz, 1 H) 6.33-6.39 (m, 1 H) 7.04-7.12 (m, 1 H) 7.21- |
| | 7.26 (m, 1 H) 7.29-7.34 (m, 1 H) 7.35-7.48 (m, 4 H) 7.67-7.71 (m, 1 |
| | H) 7.71-7.76 (m, 1 H) 7.95-8.06 (m, 2 H) 8.31-8.40 (m, 1 H) |
| C437b | 1H NMR (400 MHz, DMSO-d₆, 81° C.) δ ppm −0.20-−0.10 (m, 1 H) −0.10-−0.02 (m, 1 H) |
| | 0.14-0.26 (m, 1 H) 0.26-0.37 (m, 1 H) 0.42-0.55 (m, 1 |
| | H) 1.11 (d, J = 6.8 Hz, 3 H) 1.45-1.58 (m, 1 H) 1.62-1.75 (m, 1 H) 2.30 |
| | (d, J = 16.4 Hz, 1 H) 2.49-2.58 (m, 1 H) 2.84 (d, J = 4.6 Hz, 3 H) 3.89 (br |
| | d, J = 18.9 Hz, 1 H) 4.23-4.77 (m, 2 H) 5.17 (q, J = 7.4 Hz, 1 H) 5.57 (d, |
| | J = 7.9 Hz, 1 H) 7.14-7.31 (m, 5 H) 7.36-7.48 (m, 3 H) 7.67 (d, J = 1.9 |
| | Hz, 1 H) 7.70 (d, J = 8.2 Hz, 1 H) 8.03 (d, J = 8.6 Hz, 2 H) 8.32-8.40 (m, 1 |
| | H) |
| C437a | 1H NMR (400 MHz, DMSO-d₆, 81° C.) δ ppm −0.20-−0.11 (m, 1 H) −0.11-−0.03 (m, 1 H) |
| | 0.15-0.25 (m, 1 H) 0.28-0.37 (m, 1 H) 0.43-0.55 (m, 1 |
| | H) 1.17 (d, J = 6.8 Hz, 3 H) 1.51 (dt, J = 13.8, 6.6 Hz, 1 H) 1.68 (dt, J = |
| | 14.1, 7.2 Hz, 1 H) 2.25-2.34 (m, 1 H) 2.45-2.54 (m, 1 H) 2.84 (d, J = 4.6 |
| | Hz, 3 H) 3.96 (br d, J = 18.8 Hz, 1 H) 4.13-4.67 (m, 2 H) 5.09 (q, J = 7.2 |
| | Hz, 1 H) 5.58 (d, J = 7.7 Hz, 1 H) 7.13-7.30 (m, 5 H) 7.32-7.49 (m, 2 H) |
| | 7.36 (dd, J = 8.2, 2.0 Hz, 1 H) 7.63 (d, J = 1.9 Hz, 1 H) 7.67 (d, J = 8.2 Hz, |
| | 1 H) 8.03 (d, J = 8.7 Hz, 2 H) 8.33-8.41 (m, 1 H) |
| C438b | 1H NMR (400 MHz, DMSO-d₆, 81° C.) δ ppm 1.17 (d, J = 6.8 Hz, 3 H) |
| | 1.30 (d, J = 7.0 Hz, 3 H) 2.21-2.35 (m, 1 H) 2.41-2.53 (m, 1 H) 2.84 (d, |
| | J = 4.4 Hz, 3 H) 3.96 (br d, J = 18.8 Hz, 1 H) 4.10-4.83 (m, 2 H) 5.33-5.52 (m, 1 H) |
| | 5.66-5.82 (m, 1 H) 6.83-7.52 (m, 8 H) 7.62 (d, J = 1.9 Hz, |
| | 1 H) 7.67 (d, J = 8.2 Hz, 1 H) 8.02 (d, J = 8.8 Hz, 2 H) 8.30-8.41 (m, 1 H) |
| C438a | 1H NMR (400 MHz, DMSO-d₆, 81° C.) δ ppm 1.10 (d, J = 6.8 Hz, 3 H) |
| | 1.30 (d, J = 7.0 Hz, 3 H) 2.29 (d, J = 16.5 Hz, 1 H) 2.49-2.56 (m, 1 H) |
| | 2.84 (d, J = 4.6 Hz, 3 H) 3.89 (br d, J = 19.0 Hz, 1 H) 4.15-4.86 (m, 2 H) |
| | 5.39-5.51 (m, 1 H) 5.72 (d, J = 7.8 Hz, 1 H) 7.02-7.46 (m, 8 H) 7.67 (d, |
| | J = 1.9 Hz, 1 H) 7.70 (d, J = 8.2 Hz, 1 H) 8.01 (d, J = 8.8 Hz, 2 H) 8.31- |
| | 8.39 (m, 1 H) |
| C451 | 1H NMR (400 MHz, DMSO-d₆, 81° C.) δ ppm 1.18 (d, J = 6.8 Hz, 3 H) |
| | 2.32 (d, J = 16.3 Hz, 1 H) 2.50-2.60 (m, 1 H) 2.83 (d, J = 4.6 Hz, 3 H) |
| | 3.32-3.56 (m, 2 H) 3.69-3.88 (m, 6 H) 3.90-4.11 (m, 2 H) 4.18-4.84 |
| | (m, 2 H) 5.49-5.60 (m, 1 H) 7.30 (br d, J = 8.1 Hz, 2 H) 7.41 (dd, J = 8.2, |
| | 1.9 Hz, 1 H) 7.68 (d, J = 1.9 Hz, 1 H) 7.70 (d, J = 8.2 Hz, 1 H) 7.98 (d, J = |
| | 8.8 Hz, 2 H) 8.27-8.38 (m, 1 H) |
| C452 | 1H NMR (400 MHz, DMSO-d₆, 81° C.) δ ppm 0.97-1.11 (m, 2 H) 1.13- |
| | 1.25 (m, 3 H) 1.83-1.99 (m, 1 H) 2.34 (br d, J = 16.6 Hz, 1 H) 2.51-2.73 |
| | (m, 2 H) 2.83 (d, J = 4.6 Hz, 3 H) 3.73 (s, 3 H) 3.95-4.18 (m, 1 H) 4.22-4.95 (m, 2 H) |
| | 6.02 (br s, 1 H) 6.66-7.22 (m, 4 H) 7.32 (br d, J = 8.0 Hz, 2 |
| | H) 7.43 (dd, J = 8.2, 2.0 Hz, 1 H) 7.66-7.76 (m, 2 H) 7.96 (d, J = 8.6 Hz, 2 |
| | H) 8.26-8.37 (m, 1 H) |
| C453 | 1H NMR (400 MHz, DMSO-d₆, 100° C.) δ ppm 1.03-1.09 (m, 6 H) 1.20 |
| | (d, J = 6.8 Hz, 3 H) 2.35 (d, J = 16.3 Hz, 1 H) 2.50-2.61 (m, 1 H) 4.02 (br |
| | d, J = 18.7 Hz, 1 H) 4.12 (dq, J = 13.6, 6.7 Hz, 1 H) 4.51 (s, 2 H) 4.92 (br |
| | d, J = 7.9 Hz, 1 H) 7.28 (d, J = 8.4 Hz, 2 H) 7.42 (dd, J = 8.1, 2.0 Hz, 1 H) |
| | 7.67 (d, J = 1.8 Hz, 1 H) 7.69 (d, J = 8.1 Hz, 1 H) 8.15 (d, J = 7.9 Hz, 2 H) |
| C447a | 1H NMR (400 MHz, DMSO-d₆, 100° C.) δ ppm 1.00-1.07 (m, 7 H) 1.18 |
| | (d, J = 6.8 Hz, 3 H) 2.03 (s, 3 H) 2.35 (d, J = 16.3 Hz, 1 H) 2.50-2.58 (m, |
| | 1 H) 2.92 (d, J = 5.1 Hz, 3 H) 4.03 (br d, J = 18.7 Hz, 1 H) 4.17 (dq, J = |
| | 13.6, 6.8 Hz, 1 H) 4.52 (s, 2 H) 5.18 (br d, J = 7.9 Hz, 1 H) 6.61-6.68 (m, |
| | 1 H) 7.30 (s, 1 H) 7.41 (dd, J = 8.2, 2.0 Hz, 1 H) 7.66 (d, J = 2.0 Hz, 1 H) |
| | 7.69 (d, J = 8.1 Hz, 1 H) 7.76 (s, 1 H) |
| C447b | 1H NMR (400 MHz, DMSO-d₆, 100° C.) δ ppm 1.04 (dd, J = 11.3, 6.6 Hz, |
| | 7 H) 1.19 (d, J = 6.8 Hz, 3 H) 2.02 (s, 3 H) 2.34 (d, J = 16.4 Hz, 1 H) 2.50-2.60 (m, 1 H) |
| | 2.92 (d, J = 5.1 Hz, 3 H) 4.02 (br d, J = 18.8 Hz, 1 H) 4.17 |
| | (dq, J = 13.7, 6.8 Hz, 1 H) 4.54 (br s, 2 H) 5.18 (d, J = 7.9 Hz, 1 H) 6.65 (br |
| | d, J = 5.1 Hz, 1 H) 7.34 (s, 1 H) 7.42 (dd, J = 8.2, 2.0 Hz, 1 H) 7.66-7.71 |
| | (m, 2 H) 7.76 (s, 1 H) |
| C455a | ¹H NMR (400 MHz, DMSO-d₆, 80° C.) δ ppm 1.06 (dd, J = 6.5, 2.5 Hz, 6 H) |
| | 1.18 (d, J = 6.8 Hz, 3 H) 2.23 (s, 3 H) 2.34 (br d, J = 16.3 Hz, 1 H) 2.50- |
| | 2.58 (m, 1 H) 4.04 (br d, J = 18.7 Hz, 1 H) 4.20 (dq, J = 13.8, 6.8 Hz, 1 H) |
| | 4.33-4.77 (m, 2 H) 5.50 (d, J = 8.1 Hz, 1 H) 6.87 (br s, 1 H) 7.39 (d, J = 8.4 |
| | Hz, 1 H) 7.42 (dd, J = 8.1, 2.0 Hz, 1 H) 7.68 (d, J = 2.0 Hz, 1 H) 7.71 (d, |
| | J = 8.4 Hz, 1 H) 7.97 (dd, J = 8.3, 1.9 Hz, 1 H) 8.12 (d, J = 1.8 Hz, 1 H) 12.09-12.53 (m, 1 H) |
| C455b | 1H NMR (400 MHz, DMSO-d₆) δ ppm 1.02-1.11 (m, 6 H) 1.14-1.22 (m, |
| | 3 H) 2.18-2.27 (m, 3 H) 2.29-2.39 (m, 1 H) 2.50-2.59 (m, 1 H) 3.94-4.11 (m, 1 H) |
| | 4.13-4.29 (m, 1 H) 4.31-4.81 (m, 2 H) 5.43-5.55 (m, 1H) 6.77-7.05 (m, 1 H) 7.37-7.40 |
| | (m, 1 H) 7.40-7.45 (m, 1 H) 7.68 (s, 1H) 7.67-7.69 (m, 1 H) 7.69-7.73 (m, 1 H) 7.94-8.01 |
| | (m, 1 H) 8.12 (d, J = 1.8 Hz, 1 H) 12.05-12.59 (m, 1 H) |
| C456 | 1H NMR (400 MHz, DMSO-d₆) δ ppm 1.08-1.15 (m, 3 H) 1.11 (s, 1 H) |
| | 1.16-1.23 (m, 6 H) 2.25-2.34 (m, 1 H) 2.43-2.47 (m, 1 H) 2.57-2.66 |
| | (m, 3 H) 3.87-3.98 (m, 1 H) 3.99-4.08 (m, 1 H) 4.09-4.16 (m, 3 H) |
| | 4.27-4.65 (m, 5 H) 4.94-5.01 (m, 1 H) 7.36-7.41 (m, 1 H) 7.41-7.48 |
| | (m, 1 H) 7.64-7.66 (m, 1 H) 7.68 (s, 1 H) |
| C457 | 1H NMR (400 MHz, DMSO-d₆) δ ppm 1.19 (d, J = 6.8 Hz, 3 H) 2.34 (s, 3 |
| | H) 2.35-2.41 (m, 1 H) 2.58 (br dd, J = 16.9, 5.9 Hz, 1 H) 2.81 (d, J = 4.6 |
| | Hz, 3 H) 3.70 (s, 3 H) 4.08 (br d, J = 18.9 Hz, 1 H) 4.20 (d, J = 14.7 Hz, 1 |
| | H) 4.32 (d, J = 14.7 Hz, 1 H) 4.39-4.74 (m, 2 H) 6.66 (d, J = 1.8 Hz, 1 H) |
| | 6.69 (d, J = 7.7 Hz, 1 H) 6.77 (dd, J = 7.8, 2.1 Hz, 1 H) 7.16 (t, J = 7.8 Hz, 1 |
| | H) 7.38-7.47 (m, 3 H) 7.67-7.73 (m, 2 H) 7.89 (d, J = 8.8 Hz, 2 H) 8.25 |
| | (q, J = 4.3 Hz, 1 H) |
| C458 | ¹H NMR (400 MHz, DMSO-d₆) δ ppm 0.58-0.76 (m, 6 H) 1.10-1.29 (m, |
| | 7 H) 1.70 (dq, J = 11.80, 6.74 Hz, 1 H) 2.31-2.43 (m, 2 H) 2.57-2.71 (m, |
| | 1 H) 2.84 (d, J = 4.50 Hz, 3 H) 4.02-4.17 (m, 1 H) 4.91-4.98 (m, 1 H) |
| | 7.36 (d, J = 8.51 Hz, 2 H) 7.46 (dd, J = 8.19, 1.94 Hz, 1 H) 7.70-7.76 (m, 2 |
| | H) 7.94 (d, J = 8.76 Hz, 2 H) 8.34 (br d, J = 4.63 Hz, 1 H) |
| C459 | 1H NMR (400 MHz, DMSO-d₆) δ ppm 0.72 (d, J = 1.8 Hz, 9 H) 0.94-1.00 |
| | (m, 3 H) 1.19 (dd, J = 6.7, 2.5 Hz, 3 H) 2.34 (br d, J = 16.5 Hz, 1 H) 2.50-2.58 (m, 1 H) |
| | 2.82 (d, J = 4.4 Hz, 3 H) 3.86-4.09 (m, 2 H) 4.22 (dd, J = |
| | 22.9, 9.0 Hz, 2 H) 4.36-4.64 (m, 1 H) 7.37-7.47 (m, 3 H) 7.68 (dd, J = |
| | 1.8, 1.1 Hz, 1 H) 7.71 (dd, J = 8.1, 2.2 Hz, 1 H) 8.02 (d, J = 8.4 Hz, 2 H) |
| | 8.37 (br d, J = 4.2 Hz, 1 H) |
| C452a | 1H NMR (400 MHz, DMSO-d₆, 81° C.) δ ppm 0.99-1.09 (m, 2 H) 1.19 (d, |
| | J = 6.8 Hz, 3 H) 1.80-1.96 (m, 1 H) 2.34 (br d, J = 16.6 Hz, 1 H) 2.51-2.66 (m, 2 H) |
| | 2.83 (d, J = 4.6 Hz, 3 H) 3.73 (s, 3 H) 4.06 (br d, J = 18.4 Hz, |
| | 1 H) 4.23-4.93 (m, 2 H) 6.04 (d, J = 2.5 Hz, 1 H) 6.72 (br d, J = 8.1 Hz, 2 |
| | H) 7.08 (br d, J = 8.1 Hz, 2 H) 7.32 (br d, J = 7.9 Hz, 2 H) 7.43 (dd, J = 8.2, |
| | 2.0 Hz, 1 H) 7.70 (d, J = 1.9 Hz, 1 H) 7.72 (d, J = 8.2 Hz, 1 H) 7.96 (d, J = |
| | 8.3 Hz, 2 H) 8.27-8.35 (m, 1 H) |
| C452b | 1H NMR (400 MHz, DMSO-d₆, 81° C.) δ ppm 0.97-1.10 (m, 2 H) 1.20 (d, |
| | J = 6.8 Hz, 3 H) 1.94 (ddd, J = 9.4, 6.5, 3.4 Hz, 1 H) 2.34 (br d, J = 16.5 Hz, |
| | 1 H) 2.51-2.60 (m, 1 H) 2.62-2.74 (m, 1 H) 2.83 (d, J = 4.6 Hz, 3 H) |
| | 3.73 (s, 3 H) 4.06 (br d, J = 18.6 Hz, 1 H) 4.22-4.84 (m, 2 H) 6.02 (d, J = |
| | 2.4 Hz, 1 H) 6.79 (d, J = 8.6 Hz, 2 H) 7.13 (d, J = 8.6 Hz, 2 H) 7.33 (d, J = |
| | 8.2 Hz, 2 H) 7.43 (dd, J = 8.2, 2.0 Hz, 1 H) 7.66-7.74 (m, 2 H) 7.96 (d, J = |
| | 8.5 Hz, 2 H) 8.27-8.36 (m, 1 H) |
| C460 | 1H NMR (400 MHz, DMSO-d₆, 100° C.) δ ppm 1.08 (dd, J = 6.6, 3.1 Hz, 6 |
| | H) 1.20 (d, J = 6.8 Hz, 3 H) 2.35 (d, J = 16.3 Hz, 1 H) 2.50-2.60 (m, 1 H) |
| | 4.03 (br d, J = 18.7 Hz, 1 H) 4.17 (dq, J = 13.5, 6.8 Hz, 1 H) 4.51 (s, 2 H) |
| | 5.21 (br d, J = 7.8 Hz, 1 H) 7.41 (dd, J = 8.2, 1.9 Hz, 1 H) 7.52-7.58 (m, 2 |
| | H) 7.66 (d, J = 1.8 Hz, 1 H) 7.69 (d, J = 8.1 Hz, 1 H) 8.07 (d, J = 8.1 Hz, 2 |
| | H) 10.02 (s, 1 H) |
| C410b | 1H NMR (400 MHz, DMSO-d₆) δ ppm 1.18 (d, J = 6.60 Hz, 3 H) 1.34 (d, |
| | J = 6.82 Hz, 3 H) 2.07 (s, 3 H) 2.31 (d, J = 16.51 Hz, 1 H) 2.53 (br d, J = |
| | 6.16 Hz, 1 H) 3.71 (s, 3 H) 3.91-4.04 (m, 1 H) 4.30-4.57 (m, 2 H) 5.11 |
| | (t, J = 7.15 Hz, 1 H) 5.26 (d, J = 7.92 Hz, 1 H) 6.78-6.83 (m, 2 H) 7.15 (d, |
| | J = 8.58 Hz, 4 H) 7.37 (dd, J = 8.25, 1.87 Hz, 1 H) 7.62-7.76 (m, 4 H) |
| | 9.85 (s, 1 H) |
| C410a | 1H NMR (400 MHz, DMSO-d₆) δ ppm 1.14 (d, J = 6.82 Hz, 3 H) 1.34 (d, |
| | J = 6.82 Hz, 3 H) 2.07 (s, 3 H) 2.31 (d, J = 16.51 Hz, 1 H) 2.49-2.58 (m, 1 |
| | H) 3.72 (s, 3 H) 3.94 (br d, J = 18.93 Hz, 1 H) 4.38-4.65 (m, 2 H) 5.14 |
| | (quin, J = 7.10 Hz, 1 H) 5.19-5.28 (m, 1 H) 6.79-6.85 (m, 2 H) 7.16 (d, |
| | J = 8.58 Hz, 4 H) 7.40 (dd, J = 8.25, 1.87 Hz, 1 H) 7.64-7.76 (m, 4 H) |
| | 9.84 (s, 1 H) |
| C461 | 1H NMR (400 MHz, CHLOROFORM-d, 27° C.) δ ppm 1.18-1.31 (m, 3 H) |
| | 2.46-2.57 (m, 1 H) 2.61-2.76 (m, 1 H) 3.03 (d, J = 4.8 Hz, 3 H) 3.50-5.44 |
| | (m, 11 H) 6.22-6.38 (m, 1 H) 7.19-7.29 (m, 1 H) 7.32-7.42 (m, 2 |
| | H) 7.48-7.58 (m, 2 H) 7.86-7.99 (m, 2 H) |
| C462a | 1H NMR (400 MHz, DMSO-d₆) δ ppm 1.10-1.16 (m, 3 H) 1.43 (d, J = 6.8 |
| | Hz, 3 H) 2.06-2.12 (m, 3 H) 2.27-2.36 (m, 1 H) 2.53-2.60 (m, 1 H) |
| | 3.87-4.06 (m, 1 H) 4.23-4.83 (m, 2 H) 5.32 (t, J = 7.3 Hz, 1 H) 5.42-5.52 (m, 1 H) |
| | 5.67-5.69 (m, 1 H) 7.13-7.25 (m, 2 H) 7.26-7.34 (m, 1 |
| | H) 7.39-7.49 (m, 2 H) 7.58-7.64 (m, 1 H) 7.66-7.70 (m, 1 H) 7.70-7.74 (m, 1 H) |
| | 7.74-7.80 (m, 2 H) 7.94-8.02 (m, 1 H) 9.92-9.99 (m, 1 |
| | H) 12.69-12.88 (m, 1 H) |
| C462b | 1H NMR (400 MHz, DMSO-d₆) δ ppm 1.15-1.23 (m, 3 H) 1.44 (d, J = 6.8 |
| | Hz, 3 H) 2.09 (s, 3 H) 2.27-2.36 (m, 1 H) 2.52-2.58 (m, 1 H) 3.93-4.10 |
| | (m, 1 H) 4.15-4.78 (m, 2 H) 5.23-5.38 (m, 1 H) 5.46-5.58 (m, 1 H) |
| | 5.67-5.70 (m, 1 H) 7.03-7.32 (m, 3 H) 7.36-7.41 (m, 1 H) 7.42-7.48 |
| | (m, 1 H) 7.56-7.62 (m, 1 H) 7.64-7.66 (m, 1 H) 7.67-7.70 (m, 1 H) |
| | 7.73-7.80 (m, 2 H) 7.96 (s, 1 H) 9.93-10.01 (m, 1 H) 12.70-12.84 (m, |
| | 1 H) |
| C459a | 1H NMR (400 MHz, DMSO-d₆) δ ppm 0.72 (s, 9 H) 0.97 (d, J = 6.8 Hz, 3 |
| | H) 1.19 (d, J = 6.6 Hz, 3H) 2.34 (br d, J = 16.3 Hz, 1 H) 2.51-2.58 (m, 1 |
| | H) 2.83 (d, J = 4.4 Hz, 3 H) 3.90-4.09 (m, 2 H) 4.23 (br d, J = 9.0 Hz, 1 H) |
| | 4.36-4.64 (m, 1 H) 7.42 (br dd, J = 8.1, 2.0 Hz, 3 H) 7.68 (d, J = 2.0 Hz, 1 |
| | H) 7.70 (d, J = 8.1 Hz, 1 H) 8.02 (d, J = 8.1 Hz, 2 H) 8.36 (br d, J = 4.6 Hz, |
| | 1 H) |
| C459b | 1H NMR (400 MHz, DMSO-d₆) δ ppm 0.72 (s, 9 H) 0.92-0.99 (m, 3 H) |
| | 1.18 (d, J = 6.8 Hz, 3 H) 2.34 (br d, J = 16.3 Hz, 1 H) 2.55 (br dd, J = 16.6, |
| | 6.1 Hz, 1 H) 2.83 (d, J = 4.6 Hz, 3 H) 3.86-3.97 (m, 1 H) 4.02 (br d, J = |
| | 18.7 Hz, 1 H) 4.18 (d, J = 9.0 Hz, 1 H) 4.38-4.66 (m, 1 H) 7.37-7.46 (m, |
| | 3 H) 7.68 (d, J = 1.8 Hz, 1 H) 7.71 (d, J = 8.4 Hz, 1 H) 8.02 (d, J = 8.8 Hz, 2 |
| | H) 8.37 (br d, J = 4.4 Hz, 1 H) |
| C463a | 1H NMR (400 MHz, DMSO-d₆, 100° C.) δ ppm 1.18 (d, J = 6.8 Hz, 3 H) |
| | 1.38 (d, J = 7.0 Hz, 3 H) 2.33 (d, J = 16.5 Hz, 1 H) 2.50-2.57 (m, 1 H) |
| | 3.93-4.05 (m, 1 H) 4.45 (br s, 2 H) 5.25 (quin, J = 7.2 Hz, 1 H) 5.86 (d, |
| | J = 7.8 Hz, 1 H) 6.55 (dd, J = 2.5, 1.7 Hz, 1 H) 7.20 (d, J = 8.2 Hz, 2 H) |
| | 7.34-7.44 (m, 5 H) 7.63 (d, J = 2.0 Hz, 1 H) 7.66 (d, J = 8.1 Hz, 1 H) 7.76 |
| | (d, J = 1.8 Hz, 1 H) 8.00 (d, J = 8.1 Hz, 2 H) 8.48 (d, J = 2.5 Hz, 1 H) |
| C475 | 1H NMR (400 MHz, DMSO-d₆, 101° C.) δ ppm 1.07 (dd, J = 6.5, 3.2 Hz, 5 |
| | H) 1.21 (d, J = 6.8 Hz, 3 H) 2.36 (d, J = 16.4 Hz, 1 H) 2.57 (br dd, J = 16.3, |
| | 6.2 Hz, 1 H) 2.85 (d, J = 4.5 Hz, 3 H) 4.04 (br d, J = 18.7 Hz, 1 H) 4.09-4.21 (m, 1 H) |
| | 4.41-4.65 (m, 2 H) 4.94 (br d, J = 3.4 Hz, 1 H) 7.26 (t, J = |
| | 73.1 Hz, 1 H) 7.34 (d, J = 8.4 Hz, 2 H) 7.41-7.51 (m, 1 H) 7.42-7.50 (m, |
| | 1 H) 7.65 (d, J = 1.9 Hz, 1 H) 8.00 (d, J = 8.6 Hz, 2 H) 8.26 (br d, J = 5.5 |
| | Hz, 1 H) |
| C525 | 1H NMR (400 MHz, DMSO-d₆, 101° C.) δ ppm 1.03-1.08 (m, 5 H) 1.22 |
| | (d, J = 6.8 Hz, 3 H) 2.36 (br d, J = 16.4 Hz, 1 H) 2.56 (br d, J = 6.3 Hz, 1 H) |
| | 2.85 (d, J = 4.6 Hz, 3 H) 4.05 (br d, J = 18.8 Hz, 1 H) 4.10-4.19 (m, 1 H) |
| | 4.38-4.68 (m, 2 H) 4.94 (br d, J = 7.8 Hz, 1 H) 7.34 (br d, J = 8.0 Hz, 2 H) |
| | 7.54 (dd, J = 8.4, 1.8 Hz, 1 H) 7.62 (dd, J = 8.4, 1.1 Hz, 1 H) 7.74 (d, J = |
| | 2.0 Hz, 1 H) 8.00 (d, J = 8.1 Hz, 2 H) 8.25 (br d, J = 5.5 Hz, 1 H) |
| C526a | 1H NMR (400 MHz, DMSO-d₆, 126° C.) δ ppm 1.25 (d, J = 6.8 Hz, 3 H) |
| | 2.10 (q, J = 5.8 Hz, 2 H) 2.40 (d, J = 16.5 Hz, 1 H) 2.61 (br dd, J = 16.4, 6.2 |
| | Hz, 1 H) 2.82 (d, J = 4.6 Hz, 3 H) 4.03-4.20 (m, 3 H) 4.48-4.66 (m, 2 H) |
| | 5.29 (q, J = 6.9 Hz, 1 H) 5.54 (d, J = 7.8 Hz, 1 H) 6.69 (dd, J = 8.2, 1.2 Hz, |
| | 1 H) 6.82 (td, J = 7.5, 1.2 Hz, 1 H) 7.04-7.11 (m, 1 H) 7.17 (d, J = 7.6 Hz, |
| | 1 H) 7.39 (d, J = 8.5 Hz, 1 H) 7.43 (dd, J = 8.2, 1.9 Hz, 1 H) 7.67 (d, J = 1.9 |
| | Hz, 1 H) 7.70 (d, J = 8.2 Hz, 1 H) 7.95 (d, J = 8.8 Hz, 2 H) 8.09 (br d, J = |
| | 5.5 Hz, 1 H) |
| C526b | 1H NMR (400 MHz, DMSO-d₆, 101° C.) δ ppm 1.23 (d, J = 6.8 Hz, 3 H) |
| | 2.03-2.15 (m, 2 H) 2.39 (d, J = 16.4 Hz, 1 H) 2.60 (br dd, J = 16.5, 5.9 Hz, |
| | 1 H) 2.81 (d, J = 4.6 Hz, 2 H) 4.06 (br d, J = 18.8 Hz, 1 H) 4.11-4.20 (m, 2 |
| | H) 4.42-4.71 (m, 2 H) 5.33 (q, J = 7.1 Hz, 1 H) 5.75 (d, J = 7.9 Hz, 1 H) |
| | 6.69 (dd, J = 8.2, 1.2 Hz, 1 H) 6.81 (td, J = 7.5, 1.2 Hz, 1 H) 7.08 (td, J = |
| | 7.7, 1.7 Hz, 1 H) 7.15 (d, J = 7.6 Hz, 1 H) 7.39 (d, J = 8.5 Hz, 2 H) 7.43 |
| | (dd, J = 8.2, 2.0 Hz, 1 H) 7.69 (d, J = 1.9 Hz, 1 H) 7.71 (d, J = 8.2 Hz, 1 H) |
| | 7.94 (d, J = 8.8 Hz, 2 H) 8.20 (br d, J = 5.1 Hz, 1 H) |
| C527a | 1H NMR (400 MHz, DMSO-d₆, 101° C.) δ ppm 1.21 (d, J = 6.8 Hz, 3 H) |
| | 2.03 (q, J = 5.7 Hz, 2 H) 2.37 (d, J = 16.5 Hz, 1 H) 2.57 (br dd, J = 16.5, 5.9 |
| | Hz, 1 H) 2.79 (d, J = 4.6 Hz, 3 H) 3.66 (s, 3 H) 4.03 (s, 1 H) 4.05-4.14 |
| | (m, 2 H) 4.35-4.69 (m, 2 H) 5.18 (q, J = 6.4 Hz, 1 H) 5.49 (d, J = 7.5 Hz, 1 |
| | H) 6.24 (d, J = 2.6 Hz, 1 H) 6.41 (dd, J = 8.6, 2.6 Hz, 1 H) 7.04 (d, J = 8.4 |
| | Hz, 1 H) 7.37 (br d, J = 8.1 Hz, 2 H) 7.41 (dd, J = 8.1, 2.0 Hz, 1 H) 7.66 (d, |
| | J = 1.8 Hz, 1 H) 7.69 (d, J = 8.1 Hz, 1 H) 7.92 (d, J = 7.9 Hz, 2 H) 8.18 (q, |
| | J = 4.4 Hz, 1 H) |
| C527b | 1H NMR (400 MHz, DMSO-d₆, 126° C.) δ ppm 1.24 (d, J = 6.8 Hz, 3 H) |
| | 2.07 (q, J = 5.7 Hz, 2 H) 2.40 (d, J = 16.5 Hz, 1 H) 2.57-2.66 (m, 1 H) |
| | 2.83 (d, J = 4.6 Hz, 3 H) 3.69 (s, 3 H) 4.02-4.18 (m, 3 H) 4.49-4.66 (m, |
| | 2 H) 5.22 (q, J = 6.6 Hz, 1 H) 5.35 (d, J = 7.5 Hz, 1 H) 6.26 (d, J = 2.5 Hz, 1 |
| | H) 6.43 (dd, J = 8.5, 2.6 Hz, 1 H) 7.05 (dd, J = 8.6, 0.7 Hz, 1 H) 7.38 (d, J = |
| | 8.6 Hz, 2 H) 7.43 (dd, J = 8.2, 2.0 Hz, 1 H) 7.67 (d, J = 1.9 Hz, 1 H) 7.70 |
| | (d, J = 8.2 Hz, 1 H) 7.94 (d, J = 8.4 Hz, 2 H) 8.09 (br d, J = 5.5 Hz, 1 H) |
| C528a | 1H NMR (400 MHz, DMSO-d₆) δ ppm 1.15-1.21 (m, 3 H) 1.36-1.45 (m, |
| | 3 H) 2.08-2.13 (m, 3 H) 2.28-2.36 (m, 1 H) 2.52-2.53 (m, 1 H) 2.76-2.84 (m, 3 H) |
| | 3.89-4.06 (m, 1 H) 4.17-4.77 (m, 2 H) 5.19-5.31 (m, 1 |
| | H) 5.66-5.74 (m, 1 H) 7.11-7.30 (m, 2 H) 7.30-7.36 (m, 2 H) 7.36-7.42 (m, 1 H) |
| | 7.64-7.67 (m, 1 H) 7.68-7.71 (m, 1 H) 7.72-7.76 (m, 2 |
| | H) 7.76-7.82 (m, 2 H) 8.06-8.15 (m, 1 H) 9.94-10.00 (m, 1 H) |
| C528b | 1H NMR (400 MHz, DMSO-d₆) δ ppm 1.10-1.17 (m, 3 H) 1.36-1.43 (m, |
| | 3 H) 2.08-2.12 (m, 3 H) 2.28-2.36 (m, 1 H) 2.56-2.59 (m, 1 H) 2.77- |
| | 2.82 (m, 3 H) 3.85-4.00 (m, 1 H) 4.12-4.92 (m, 2 H) 5.22-5.33 (m, 1 |
| | H) 5.64-5.72 (m, 1 H) 7.18-7.25 (m, 2 H) 7.31-7.39 (m, 2 H) 7.39- |
| | 7.46 (m, 1 H) 7.68-7.70 (m, 1 H) 7.71-7.73 (m, 1 H) 7.73-7.76 (m, 2 |
| | H) 7.76-7.81 (m, 2 H) 8.08-8.16 (m, 1 H) 9.95-9.99 (m, 1 H) |
| C529a | 1H NMR (400 MHz, DMSO-d₆) δ ppm 1.17-1.24 (m, 3 H) 1.40-1.49 (m, |
| | 3 H) 2.28-2.37 (m, 1 H) 2.53-2.56 (m, 1 H) 2.81-2.88 (m, 3 H) 3.95-4.14 (m, 1 H) |
| | 4.25-4.76 (m, 2 H) 5.26-5.45 (m, 1 H) 5.63-5.78 (m, 1 |
| | H) 7.25-7.32 (m, 1 H) 7.32-7.49 (m, 4 H) 7.58-7.63 (m, 1 H) 7.65-7.67 (m, 1 H) |
| | 7.67-7.72 (m, 1 H) 7.93-7.98 (m, 1 H) 7.98-8.06 (m, 2 |
| | H) 8.29-8.39 (m, 1 H) 12.67-12.87 (m, 1 H) |
| C529b | 1H NMR (400 MHz, DMSO-d₆) δ ppm 1.11-1.18 (m, 3 H) 1.40-1.48 (m, |
| | 3 H) 2.28-2.37 (m, 1 H) 2.54-2.58 (m, 1 H) 2.83-2.87 (m, 3 H) 3.90-4.07 (m, 1 H) |
| | 4.17-4.90 (m, 2 H) 5.31-5.43 (m, 1 H) 5.65-5.74 (m, 1 |
| | H) 7.28-7.34 (m, 1 H) 7.37-7.42 (m, 2 H) 7.42-7.48 (m, 2 H) 7.61-7.64 (m, 1 H) |
| | 7.69-7.71 (m, 1 H) 7.71-7.76 (m, 1 H) 7.97-7.99 (m, 1 |
| | H) 7.99-8.07 (m, 2 H) 8.31-8.39 (m, 1 H) 12.78 (br s, 1 H) |
| C463b | 1H NMR (400 MHz, DMSO-d₆, 100° C.) δ ppm 1.14 (d, J = 6.8 Hz, 3 H) |
| | 1.39 (d, J = 7.0 Hz, 3 H) 2.29-2.45 (m, 1 H) 2.51-2.60 (m, 1 H) 3.92 (br |
| | d, J = 18.8 Hz, 1 H) 4.50 (s, 2 H) 5.29 (quin, J = 7.2 Hz, 1 H) 5.86 (d, J = |
| | 7.9 Hz, 1 H) 6.55 (s, 1 H) 7.22 (d, J = 8.2 Hz, 2 H) 7.36-7.46 (m, 5 H) |
| | 7.65 (d, J = 2.0 Hz, 1 H) 7.69 (d, J = 8.1 Hz, 1 H) 7.76 (d, J = 1.8 Hz, 1 H) |
| | 8.00 (d, J = 7.9 Hz, 2 H) 8.48 (d, J = 2.6 Hz, 1 H) |
| C530a | 1H NMR (400 MHz, DMSO-d₆) δ ppm 1.18-1.26 (m, 3 H) 1.39-1.46 (m, |
| | 3 H) 2.29-2.39 (m, 1 H) 2.55-2.57 (m, 1 H) 2.79-2.85 (m, 3 H) 2.88 (d, |
| | J = 4.6 Hz, 3 H) 3.94-4.11 (m, 1 H) 4.16-4.87 (m, 2 H) 5.24-5.36 (m, 1 |
| | H) 5.82-5.93 (m, 1 H) 7.33-7.38 (m, 2 H) 7.39-7.47 (m, 3 H) 7.66-7.70 (m, 1 H) |
| | 7.70-7.74 (m, 1 H) 7.74-7.80 (m, 2 H) 8.00-8.07 (m, 2 |
| | H) 8.08-8.16 (m, 1 H) 8.34-8.42 (m, 1 H) |
| C530b | 1H NMR (400 MHz, DMSO-d₆) δ ppm 1.13-1.21 (m, 3 H) 1.37-1.47 (m, |
| | 3 H) 2.30-2.40 (m, 1 H) 2.55-2.57 (m, 1 H) 2.79-2.84 (m, 3 H) 2.85-2.92 (m, 3 H) |
| | 3.86-4.06 (m, 1 H) 4.16-4.87 (m, 2 H) 5.24-5.42 (m, 1 |
| | H) 5.81-5.94 (m, 1 H) 7.34-7.40 (m, 2 H) 7.41-7.47 (m, 3 H) 7.70-7.73 (m, 1 H) |
| | 7.73-7.76 (m, 1 H) 7.76-7.80 (m, 2 H) 8.01-8.08 (m, 2 |
| | H) 8.10-8.19 (m, 1 H) 8.34-8.45 (m, 1 H) |
| C531 | 1H NMR (400 MHz, CHLOROFORM-d, 27° C.) δ ppm 1.06-1.44 (m, 6 H) |
| | 2.37-3.10 (m, 5 H) 3.62-5.28 (m, 4 H) 6.50-6.66 (m, 1 H) 7.02-7.35 |
| | (m, 5 H) 7.44-7.58 (m, 2 H) 7.65-8.13 (m, 1 H) |
| C532 | 1H NMR (400 MHz, DMSO-d₆, 100° C.) δ ppm 1.18 (d, J = 6.8 Hz, 3 H) |
| | 1.40 (d, J = 7.1 Hz, 3 H) 2.33 (d, J = 16.5 Hz, 1 H) 2.50-2.56 (m, 1 H) |
| | 3.97 (br d, J = 18.8 Hz, 1 H) 4.44 (br s, 2 H) 5.29 (quin, J = 7.2 Hz, 1 H) |
| | 5.95 (d, J = 7.7 Hz, 1 H) 6.55 (dd, J = 2.5, 1.7 Hz, 1 H) 7.36 (dd, J = 8.1, |
| | 2.0 Hz, 1 H) 7.41 (br d, J = 8.4 Hz, 2 H) 7.47-7.52 (m, 2 H) 7.57-7.61 |
| | (m, 2 H) 7.62 (d, J = 2.0 Hz, 1 H) 7.65 (d, J = 8.1 Hz, 1 H) 7.76 (d, J = 1.7 |
| | Hz, 1 H) 8.01 (d, J = 8.2 Hz, 2 H) 8.48 (d, J = 2.5 Hz, 1 H) |
| C533 | 1H NMR (400 MHz, DMSO-d₆, 100° C.) δ ppm 1.18 (d, J = 6.8 Hz, 3 H) |
| | 1.40 (d, J = 7.1 Hz, 3 H) 2.29-2.36 (m, 1 H) 2.51-2.57 (m, 1 H) 2.89 (d, |
| | J = 4.8 Hz, 3 H) 3.98 (br d, J = 18.9 Hz, 1 H) 4.30-4.55 (m, 2 H) 5.30 |
| | (quin, J = 7.2 Hz, 1 H) 6.39 (d, J = 7.6 Hz, 1 H) 7.36 (dd, J = 8.2, 2.0 Hz, 1 |
| | H) 7.47-7.53 (m, 2 H) 7.59 (d, J = 8.1 Hz, 1 H) 7.61 (d, J = 1.8 Hz, 1 H) |
| | 7.65 (d, J = 8.1 Hz, 1 H) 7.89-7.99 (m, 1 H) 8.19 (d, J = 8.2 Hz, 1 H) 8.50-8.60 (m, 2 H) |
| C534 | 1H NMR (400 MHz, DMSO-d₆) δ ppm 1.16 (dd, J = 9.5, 6.9 Hz, 3 H) 1.44 |
| | (dd, J = 7.1, 4.3 Hz, 3 H) 2.25-2.35 (m, 1 H) 2.50-2.55 (m, 1 H) 2.79 |
| | (dd, J = 4.7, 0.7 Hz, 3 H) 3.59 (d, J = 31.7 Hz, 3 H) 3.91-4.02 (m, 1 H) |
| | 4.27-4.54 (m, 1 H) 5.20-5.34 (m, 1 H) 6.73 (d, J = 19.3 Hz, 1 H) 6.89 (br |
| | d, J = 7.1 Hz, 1 H) 7.36 (dd, J = 8.2, 1.9 Hz, 1 H) 7.50-7.57 (m, 2 H) 7.57- |
| | 7.68 (m, 3 H) 7.66-7.77 (m, 1 H) 7.84-7.92 (m, 1 H) 8.01-8.06 (m, 1 |
| | H) |
| C535a | 1H NMR (400 MHz, DMSO-d₆) δ ppm 1.19 (d, J = 6.8 Hz, 3 H) 2.33 (d, J = |
| | 16.7 Hz, 1 H) 2.50-2.58 (m, 1 H) 2.84 (d, J = 4.4 Hz, 3 H) 4.05 (br d, J = |
| | 17.8 Hz, 1 H) 4.35-4.63 (m, 2 H) 6.05-6.20 (m, 2 H) 7.31-7.44 (m, 8 |
| | H) 7.65 (d, J = 1.8 Hz, 1 H) 7.69 (d, J = 8.4 Hz, 1 H) 8.01 (d, J = 8.6 Hz, 2 |
| | H) 8.36 (br d, J = 4.4 Hz, 1 H) |
| C535b | 1H NMR (400 MHz, DMSO-d₆) δ ppm 1.13 (d, J = 6.8 Hz, 3 H) 2.29-2.36 |
| | (m, 1 H) 2.51-2.60 (m, 2 H) 2.84 (d, J = 4.6 Hz, 3 H) 3.95-4.06 (m, 1 H) |
| | 4.39-4.67 (m, 1 H) 6.12-6.21 (m, 2 H) 7.35-7.45 (m, 8 H) 7.69 (d, J = |
| | 2.0 Hz, 1 H) 7.71 (d, J = 8.1 Hz, 1 H) 8.02 (d, J = 8.4 Hz, 2 H) 8.34-8.40 |
| | (m, 1 H) |
| C536 | 1H NMR (400 MHz, DMSO-d₆) δ ppm 1.17 (d, J = 6.8 Hz, 3 H) 2.32 (d, J = |
| | 16.3 Hz, 1 H) 2.50-2.58 (m, 1 H) 2.82 (d, J = 4.6 Hz, 3 H) 3.97 (br d, J = |
| | 19.1 Hz, 1 H) 4.33-4.56 (m, 4 H) 6.35 (br s, 1 H) 7.16-7.29 (m, 5 H) |
| | 7.33-7.41 (m, 3 H) 7.66 (d, J = 2.0 Hz, 1 H) 7.68 (d, J = 8.1 Hz, 1 H) 7.98 |
| | (d, J = 7.9 Hz, 2 H) 8.31 (br d, J = 4.4 Hz, 1 H) |
| C537 | 1H NMR (400 MHz, DMSO-d₆, 100° C.) δ ppm 1.18 (d, J = 6.8 Hz, 3 H) |
| | 1.38 (d, J = 7.1 Hz, 3 H) 2.32 (d, J = 16.4 Hz, 1 H) 2.54 (br d, J = 6.3 Hz, 1 |
| | H) 2.88 (d, J = 5.1 Hz, 3 H) 3.99 (br d, J = 18.9 Hz, 1 H) 4.35-4.54 (m, 2 |
| | H) 5.27 (quin, J = 7.2 Hz, 1 H) 6.33 (d, J = 7.7 Hz, 1 H) 7.20 (d, J = 8.2 Hz, |
| | 2 H) 7.36 (dd, J = 8.1, 2.0 Hz, 1 H) 7.39 (d, J = 8.6 Hz, 2 H) 7.62 (d, J = 1.8 |
| | Hz, 1 H) 7.66 (d, J = 8.1 Hz, 1 H) 7.93 (br d, J = 8.3 Hz, 1 H) 8.18 (dd, J = |
| | 8.3, 0.8 Hz, 1 H) 8.52 (d, J = 2.4 Hz, 1 H) 8.56 (br d, J = 5.3 Hz, 1 H) |
| C606 | 1H NMR (400 MHz, DMSO-d₆, 101° C.) δ ppm 1.09 (dd, J = 6.6, 2.1 Hz, 6 |
| | H) 1.21 (br d, J = 6.7 Hz, 3 H) 2.34-2.44 (m, 1 H) 2.53-2.62 (m, 1 H) |
| | 2.85 (d, J = 4.6 Hz, 3 H) 3.43-4.08 (m, 2 H) 4.10-4.18 (m, 1 H) 4.19-4.31 (m, 6 H) |
| | 4.42-4.86 (m, 1 H) 4.92 (br d, J = 7.7 Hz, 1 H) 7.34 (d, J = |
| | 8.3 Hz, 2 H) 7.99 (d, J = 8.4 Hz, 2 H) 8.21-8.30 (m, 1 H) |
| C625 | 1H NMR (400 MHz, DMSO-d₆, 100° C.) δ ppm 0.78 (t, J = 7.0 Hz, 6 H) |
| | 1.19 (d, J = 6.8 Hz, 3 H) 2.38 (d, J = 16.8 Hz, 1 H) 2.51-2.65 (m, 1 H) |
| | 2.86 (d, J = 4.8 Hz, 3 H) 2.97-3.07 (m, 4 H) 4.09 (br d, J = 19.0 Hz, 1 H) |
| | 4.42-4.69 (m, 2 H) 7.41 (dd, J = 8.2, 2.0 Hz, 1 H) 7.66 (d, J = 2.0 Hz, 1 H) |
| | 7.69 (d, J = 8.4 Hz, 1 H) 7.98 (dd, J = 8.3, 2.4 Hz, 1 H) 8.14 (d, J = 8.3 Hz, |
| | 1 H) 8.49 (br d, J = 5.7 Hz, 1 H) 8.56 (d, J = 2.4 Hz, 1 H) |
| C607 | 1H NMR (400 MHz, DMSO-d₆, 81° C.) δ ppm 1.16 (d, J = 6.9 Hz, 3 H) |
| | 1.40 (d, J = 7.0 Hz, 3 H) 2.30 (d, J = 16.3 Hz, 1 H) 2.43-2.58 (m, 1 H) |
| | 2.81 (d, J = 4.7 Hz, 3 H) 3.96 (br d, J = 19.1 Hz, 1 H) 4.20-4.62 (m, 2 H) |
| | 5.24 (quin, J = 7.2 Hz, 1 H) 6.41 (br d, J = 7.8 Hz, 1 H) 6.81 (s, 1 H) 7.22 |
| | (br d, J = 8.2 Hz, 2 H) 7.37 (dd, J = 8.2, 2.0 Hz, 1 H) 7.40 (br d, J = 8.7 Hz, |
| | 2 H) 7.64 (d, J = 1.9 Hz, 1 H) 7.67 (d, J = 8.2 Hz, 1 H) 8.19-8.35 (m, 1 H) |
| | 12.92-14.06 (m, 1 H) |
| C608b | 1H NMR (400 MHz, DMSO-d₆, 81° C.) δ ppm 1.02-1.16 (m, 6 H) 2.26 (br |
| | d, J = 16.4 Hz, 1 H) 2.44-2.55 (m, 1 H) 2.73-2.90 (m, 5 H) 3.88 (br d, J = |
| | 18.8 Hz, 1 H) 4.15-4.85 (m, 3 H) 5.21 (br d, J = 8.3 Hz, 1 H) 7.08-7.28 |
| | (m, 2 H) 7.31 (br d, J = 7.9 Hz, 2 H) 7.40 (dd, J = 8.2, 2.0 Hz, 1 H) 7.58 (br |
| | d, J = 7.9 Hz, 2 H) 7.67 (d, J = 1.9 Hz, 1 H) 7.70 (d, J = 8.2 Hz, 1 H) 7.98 |
| | (br d, J = 8.6 Hz, 2 H) 8.29-8.39 (m, 1 H) |
| C609 | 1H NMR (400 MHz, DMSO-d₆, 81° C.) δ ppm 1.17 (d, J = 6.8 Hz, 3 H) 1.37 |
| | (d, J = 7.0 Hz, 3 H) 2.26-2.34 (m, 1 H) 2.47-2.55 (m, 1 H) 2.83 (d, J = |
| | 4.6 Hz, 3 H) 3.97 (br d, J = 18.8 Hz, 1 H) 4.18-4.75 (m, 2 H) 5.25 (quin, |
| | J = 7.2 Hz, 1 H) 5.87 (br d, J = 7.7 Hz, 1 H) 6.91 (t, J = 56.1 Hz, 1 H) 7.31-7.50 |
| | (m, 7 H) 7.64 (d, J = 1.9 Hz, 1 H) 7.67 (d, J = 8.2 Hz, 1 H) 8.00 (d, J = |
| | 8.8 Hz, 2 H) 8.28-8.37 (m, 1 H) |
| C610 | 1H NMR (400 MHz, DMSO-d₆, 100° C.) δ ppm 1.06 (dd, J = 6.4, 2.9 Hz, 6 |
| | H) 1.19 (d, J = 6.8 Hz, 3 H) 2.31-2.45 (m, 1 H) 2.50-2.59 (m, 1 H) 4.02 |
| | (br d, J = 18.7 Hz, 1 H) 4.14 (dq, J = 13.6, 6.7 Hz, 1 H) 4.51 (s, 2 H) 4.99 |
| | (br d, J = 7.8 Hz, 1 H) 7.34 (br d, J = 8.1 Hz, 2 H) 7.41 (dd, J = 8.3, 1.9 Hz, |
| | 1 H) 7.66 (d, J = 2.0 Hz, 1 H) 7.69 (d, J = 8.1 Hz, 1 H) 8.17 (d, J = 7.7 Hz, 2 |
| | H) 8.39 (br s, 1 H) 14.02 (br s, 1 H) |
| C608a | 1H NMR (400 MHz, DMSO-d₆, 81° C.) δ ppm 1.09 (d, J = 6.6 Hz, 3 H) 1.16 |
| | (d, J = 6.8 Hz, 3 H) 2.23-2.34 (m, 1 H) 2.40-2.50 (m, 1 H) 2.69-2.79 |
| | (m, 1 H) 2.80-2.90 (m, 4 H) 4.00 (br d, J = 18.5 Hz, 1 H) 4.22-4.73 (m, 3 |
| | H) 5.22 (br d, J = 8.4 Hz, 1 H) 6.93-7.38 (m, 4 H) 7.41 (dd, J = 8.1, 2.0 |
| | Hz, 1 H) 7.55 (br d, J = 7.9 Hz, 2 H) 7.67 (d, J = 1.9 Hz, 1 H) 7.71 (d, J = |
| | 8.2 Hz, 1 H) 7.97 (d, J = 8.8 Hz, 2 H) 8.29-8.37 (m, 1 H) |
| C611 | 1H NMR (400 MHz, DMSO-d₆, 100° C.) δ ppm 1.07 (dd, J = 6.4, 3.1 Hz, 6 |
| | H) 1.19 (d, J = 6.6 Hz, 3 H) 2.34 (d, J = 16.4 Hz, 1 H) 2.50-2.60 (m, 1 H) |
| | 4.02 (br d, J = 18.7 Hz, 1 H) 4.09-4.23 (m, 1 H) 4.51 (br s, 2 H) 5.13 (br |
| | d, J = 7.8 Hz, 1 H) 7.41 (dd, J = 8.1, 2.0 Hz, 1 H) 7.43-7.48 (m, 2 H) 7.66 |
| | (d, J = 1.8 Hz, 1 H) 7.69 (d, J = 8.1 Hz, 1 H) 7.99-8.04 (m, 2 H) 8.20 (s, 1 |
| | H) 9.25 (s, 1 H) |
| C612 | 1H NMR (400 MHz, DMSO-d₆, 100° C.) δ ppm 1.18 (d, J = 6.8 Hz, 3 H) |
| | 1.37 (d, J = 7.0 Hz, 3 H) 2.28-2.45 (m, 1 H) 2.51-2.61 (m, 1 H) 2.93 (d, |
| | J = 5.1 Hz, 3 H) 3.97 (br d, J = 18.9 Hz, 1 H) 4.43 (br s, 2 H) 5.23-5.34 |
| | (m, 1 H) 6.00 (br d, J = 7.7 Hz, 1 H) 6.71-6.79 (m, 1 H) 7.10 (s, 1 H) 7.36 |
| | (dd, J = 8.2, 2.0 Hz, 1 H) 7.48 (br d, J = 8.6 Hz, 3 H) 7.58 (br d, J = 7.9 Hz, |
| | 2 H) 7.62 (d, J = 2.0 Hz, 1 H) 7.65 (d, J = 8.1 Hz, 1 H) 7.92 (d, J = 8.3 Hz, 1 |
| | H) |
| C613 | 1H NMR (400 MHz, DMSO-d₆) δ ppm 1.17-1.22 (m, 3 H) 1.34-1.38 (m, |
| | 3 H) 2.28-2.37 (m, 1 H) 2.53-2.54 (m, 1 H) 2.83-2.88 (m, 3 H) 3.91-4.10 (m, 1 H) |
| | 4.21-4.73 (m, 2 H) 5.21 (s, 1 H) 5.85 (d, J = 7.5 Hz, 1 H) |
| | 7.24-7.33 (m, 4 H) 7.33-7.43 (m, 3 H) 7.65-7.68 (m, 1 H) 7.68-7.73 |
| | (m, 1 H) 7.99-8.05 (m, 2 H) 8.30-8.42 (m, 1 H) |
| C614 | 1H NMR (400 MHz, DMSO-d₆, 81° C.) δ ppm 1.18 (d, J = 6.8 Hz, 3 H) |
| | 1.36 (d, J = 7.1 Hz, 3 H) 2.30 (br d, J = 16.3 Hz, 1 H) 2.48-2.57 (m, 1 H) |
| | 2.89 (d, J = 4.8 Hz, 3 H) 4.00 (br d, J = 19.0 Hz, 1 H) 4.20-4.70 (m, 2 H) |
| | 5.28 (quin, J = 7.2 Hz, 1 H) 6.63 (d, J = 7.7 Hz, 1 H) 7.00-7.11 (m, 2 H) |
| | 7.26-7.35 (m, 2 H) 7.38 (dd, J = 8.2, 2.0 Hz, 1 H) 7.64 (d, J = 1.9 Hz, 1 H) |
| | 7.68 (d, J = 8.2 Hz, 1 H) 8.74-8.81 (m, 1 H) 8.82 (d, J = 1.4 Hz, 1 H) 9.22 |
| | (d, J = 1.5 Hz, 1 H) |
| C615 | 1H NMR (400 MHz, DMSO-d₆, 81° C.) δ ppm 1.17 (d, J = 6.8 Hz, 3 H) |
| | 1.39 (d, J = 7.3 Hz, 3 H) 2.30 (d, J = 16.4 Hz, 1 H) 2.48-2.57 (m, 1 H) |
| | 2.90 (d, J = 4.8 Hz, 3 H) 3.99 (br d, J = 18.9 Hz, 1 H) 4.14-4.83 (m, 2 H) |
| | 5.34 (quin, J = 7.1 Hz, 1 H) 6.74 (br d, J = 7.6 Hz, 1 H) 7.37 (dd, J = 8.4, |
| | 2.0 Hz, 1 H) 7.51 (br d, J = 8.0 Hz, 2 H) 7.59-7.65 (m, 3 H) 7.66 (d, J = |
| | 8.2 Hz, 1 H) 8.79 (q, J = 4.7 Hz, 1 H) 8.85 (d, J = 1.4 Hz, 1 H) 9.24 (d, J = |
| | 1.3 Hz, 1 H) |
| C616 | 1H NMR (400 MHz, DMSO-d₆, 100° C.) δ ppm 1.18 (d, J = 6.8 Hz, 3 H) |
| | 1.32-1.40 (m, 3 H) 2.32 (d, J = 16.4 Hz, 1 H) 2.50-2.57 (m, 1 H) 2.93 (d, |
| | J = 5.0 Hz, 3 H) 3.98 (br d, J = 18.8 Hz, 1 H) 4.32-4.56 (m, 2 H) 5.25 |
| | (quin, J = 7.2 Hz, 1 H) 5.92 (d, J = 7.7 Hz, 1 H) 6.74 (q, J = 4.9 Hz, 1 H) |
| | 7.09 (br s, 1 H) 7.19 (d, J = 8.3 Hz, 2 H) 7.37 (dt, J = 7.9, 2.0 Hz, 3 H) 7.45 |
| | (br s, 1 H) 7.62 (d, J = 1.9 Hz, 1 H) 7.66 (d, J = 8.2 Hz, 1 H) 7.91 (d, J = 8.3 |
| | Hz, 1 H) |
| C617 | 1H NMR (400 MHz, DMSO-d₆, 81° C.) δ ppm 1.18 (d, J = 6.8 Hz, 3 H) |
| | 1.37 (d, J = 7.0 Hz, 3 H) 2.31 (br d, J = 16.4 Hz, 1 H) 2.49-2.56 (m, 1 H) |
| | 2.90 (d, J = 4.8 Hz, 3 H) 4.00 (br d, J = 18.7 Hz, 1 H) 4.18-4.75 (m, 2 H) |
| | 5.32 (quin, J = 7.2 Hz, 1 H) 6.68 (br d, J = 7.7 Hz, 1 H) 7.23 (br d, J = 8.1 |
| | Hz, 2 H) 7.35-7.45 (m, 3 H) 7.64 (d, J = 1.9 Hz, 1 H) 7.67 (d, J = 8.2 Hz, 1 |
| | H) 8.78 (q, J = 4.8 Hz, 1 H) 8.84 (d, J = 1.4 Hz, 1 H) 9.23 (d, J = 1.3 Hz, 1 |
| | H) |
| C618 | 1H NMR (400 MHz, DMSO-d₆) δ ppm 1.17 (dd, J = 9.2, 6.8 Hz, 3 H) 1.42 |
| | (dd, J = 7.2, 3.6 Hz, 3 H) 2.26-2.36 (m, 1 H) 2.50-2.58 (m, 1 H) 2.79 |
| | (dd, J = 4.8, 1.1 Hz, 3 H) 3.58 (d, J = 35.2 Hz, 3 H) 3.97 (br dd, J = 20.1, |
| | 3.0 Hz, 1 H) 4.30-4.55 (m, 1 H) 5.17-5.31 (m, 1 H) 6.69 (s, 1 H) 6.75 |
| | (s, 1 H) 6.77-6.85 (m, 1 H) 7.23 (br t, J = 7.5 Hz, 2 H) 7.37 (dd, J = 8.1, |
| | 2.0 Hz, 1 H) 7.41-7.46 (m, 2 H) 7.64 (t, J = 1.7 Hz, 1 H) 7.67 (dd, J = 8.1, |
| | 1.5 Hz, 1 H) 7.87 (br d, J = 4.4 Hz, 1 H) |
| C619 | 1H NMR (400 MHz, DMSO-d₆) δ ppm 1.07-1.30 (m, 5 H) 1.43 (dd, J = |
| | 7.0, 5.1 Hz, 2 H) 1.95-2.19 (m, 1 H) 2.22-2.35 (m, 1 H) 2.50-2.60 (m, |
| | 1 H) 2.81 (dd, J = 4.8, 2.6 Hz, 2 H) 3.59 (s, 1 H) 3.63-3.72 (m, 1 H) 3.90- |
| | 4.13 (m, 1 H) 4.41 (br s, 1 H) 5.28 (dt, J = 39.1, 7.3 Hz, 1 H) 6.87-6.95 |
| | (m, 1 H) 7.07-7.17 (m, 1 H) 7.37 (dd, J = 8.3, 1.9 Hz, 1 H) 7.50-7.71 (m, |
| | 7 H) 8.28 (br t, J = 5.3 Hz, 1 H) |
| C620 | 1H NMR (400 MHz, DMSO-d₆) δ ppm 1.13-1.21 (m, 3 H) 1.38-1.44 (m, |
| | 3 H) 2.23-2.36 (m, 1 H) 2.51-2.59 (m, 1 H) 2.77-2.85 (m, 3 H) 3.54-3.67 (m, 3 H) |
| | 3.90-4.04 (m, 1 H) 4.28-4.55 (m, 2 H) 5.17-5.33 (m, 1 |
| | H) 6.81-6.90 (m, 1 H) 7.05-7.15 (m, 1 H) 7.19-7.28 (m, 2 H) 7.35-7.39 (m, 1 H) |
| | 7.39-7.46 (m, 2 H) 7.64 (d, J = 2.0 Hz, 1 H) 7.67 (d, J = 8.4 |
| | Hz, 1 H) 8.24-8.31 (m, 1 H) |
| C621 | 1H NMR (400 MHz, DMSO-d₆) δ ppm 0.71 (s, 3 H) 0.74-0.79 (m, 1 H) |
| | 0.80 (s, 3 H) 0.90 (s, 3 H) 0.90-0.98 (m, 2 H) 1.18 (d, J = 6.8 Hz, 4 H) |
| | 1.50-1.63 (m, 2 H) 2.18-2.29 (m, 1 H) 2.29-2.38 (m, 1 H) 2.50-2.59 |
| | (m, 2 H) 2.83 (d, J = 4.6 Hz, 3 H) 3.93-4.05 (m, 1 H) 4.24-4.32 (m, 1 H) |
| | 4.37 (br d, J = 8.1 Hz, 1 H) 4.39-4.68 (m, 2 H) 7.41 (dd, J = 8.1, 2.0 Hz, 1 |
| | H) 7.45 (br d, J = 7.9 Hz, 2 H) 7.67 (d, J = 2.0 Hz, 1 H) 7.70 (d, J = 8.1 Hz, |
| | 1 H) 8.03 (d, J = 8.0 Hz, 2 H) 8.31-8.43 (m, 1 H) |
| C622 | 1H NMR (400 MHz, DMSO-d₆, 81° C.) δ ppm 1.15 (d, J = 6.8 Hz, 3 H) |
| | 1.41 (d, J = 7.1 Hz, 3 H) 2.23-2.34 (m, 1 H) 2.47-2.56 (m, 1 H) 2.79 (d, |
| | J = 4.6 Hz, 3 H) 3.96 (br d, J = 18.9 Hz, 1 H) 4.11 (s, 3 H) 4.23-4.61 (m, 2 |
| | H) 5.26 (quin, J = 7.3 Hz, 1 H) 6.45 (br d, J = 7.8 Hz, 1 H) 6.85 (s, 1 H) |
| | 7.23 (br d, J = 8.1 Hz, 2 H) 7.37 (dd, J = 8.2, 2.0 Hz, 1 H) 7.39-7.44 (m, 2 |
| | H) 7.64 (d, J = 1.9 Hz, 1 H) 7.67 (d, J = 8.2 Hz, 1 H) 8.25-8.37 (m, 1 H) |
| C623 | 1H NMR (400 MHz, DMSO-d₆, 81° C.) δ ppm 1.15 (d, J = 6.8 Hz, 3 H) |
| | 1.43 (d, J = 7.1 Hz, 3 H) 2.21-2.36 (m, 1 H) 2.42-2.58 (m, 1 H) 2.79 (d, |
| | J = 4.5 Hz, 3 H) 3.86-4.01 (m, 1 H) 4.12 (s, 3 H) 4.22-4.73 (m, 2 H) |
| | 5.29 (quin, J = 7.3 Hz, 1 H) 6.52 (br d, J = 7.7 Hz, 1 H) 6.85 (s, 1 H) 7.36 |
| | (dd, J = 8.2, 1.9 Hz, 1 H) 7.51 (d, J = 8.0 Hz, 2 H) 7.58-7.64 (m, 3 H) 7.66 |
| | (d, J = 8.2 Hz, 1 H) 8.28-8.37 (m, 1 H) |
| C624 | 1H NMR (400 MHz, DMSO-d₆, 81° C.) δ ppm 1.16 (d, J = 6.8 Hz, 3 H) |
| | 1.40 (d, J = 7.0 Hz, 3 H) 2.24-2.33 (m, 1 H) 2.49-2.57 (m, 1 H) 2.79 (d, |
| | J = 4.6 Hz, 3 H) 3.96 (br d, J = 19.1 Hz, 1 H) 4.11 (s, 3 H) 4.21-4.69 (m, 2 |
| | H) 5.21 (quin, J = 7.2 Hz, 1 H) 6.38 (br d, J = 7.9 Hz, 1 H) 6.84 (s, 1 H) |
| | 7.01-7.12 (m, 2 H) 7.28-7.35 (m, 2 H) 7.37 (dd, J = 8.2, 2.0 Hz, 1 H) |
| | 7.64 (d, J = 1.9 Hz, 1 H) 7.67 (d, J = 8.2 Hz, 1 H) 8.26-8.37 (m, 1 H) |
| C626 | 1H NMR (400 MHz, CHLOROFORM-D δ ppm 1.20-1.31 (m, 4 H) 2.47 |
| | (dd, J = 16.8, 3.6 Hz, 1 H) 2.59-2.69 (m, 1 H) 2.95 (d, J = 5.3 Hz, 3 H) |
| | 3.80-3.85 (m, 3 H) 3.92 (br d, J = 19.4 Hz, 1 H) 4.43 (br d, J = 2.4 Hz, 1 |
| | H) 5.09 (br d, J = 18.5 Hz, 1 H) 7.02 (d, J = 4.8 Hz, 1 H) 7.25 (s, 1 H) 7.26-7.28 |
| | (m, 1 H) 7.41 (br dd, J = 10.0, 5.0 Hz, 1 H) 7.52 (dd, J = 1.8, 1.1 Hz, |
| | 1 H) 7.54 (d, J = 8.1 Hz, 1 H) |
| C627 | 1H NMR (400 MHz, DMSO-d₆, 100° C.) δ ppm 1.03 (dd, J = 6.5, 2.6 Hz, 6 |
| | H) 1.19 (d, J = 6.8 Hz, 3 H) 2.30-2.38 (m, 1 H) 2.50-2.59 (m, 1 H) 2.92 |
| | (d, J = 5.3 Hz, 3 H) 4.02 (br d, J = 18.7 Hz, 1 H) 4.12 (dq, J = 13.5, 6.8 Hz, |
| | 1 H) 4.39-4.64 (m, 2 H) 4.87 (d, J = 7.8 Hz, 1 H) 5.69 (q, J = 5.0 Hz, 1 H) |
| | 6.67 (br d, J = 8.4 Hz, 1 H) 7.12 (s, 1 H) 7.41 (dd, J = 8.2, 1.9 Hz, 1 H) |
| | 7.66 (d, J = 2.0 Hz, 1 H) 7.69 (d, J = 8.4 Hz, 1 H) 7.79 (d, J = 8.4 Hz, 1 H) |
| | 11.38 (s, 1 H) |
| C628 | 1H NMR (400 MHz, DMSO-d₆, 81° C.) δ ppm 1.10 (d, J = 6.8 Hz, 3 H) |
| | 1.31 (d, J = 7.0 Hz, 3 H) 2.24 (br d, J = 16.4 Hz, 1 H) 2.35-2.50 (m, 1 H) |
| | 2.76 (d, J = 4.6 Hz, 3 H) 3.91 (br d, J = 18.8 Hz, 1 H) 4.06-4.72 (m, 2 H) |
| | 5.20 (quin, J = 7.0 Hz, 1 H) 5.89 (br d, J = 7.6 Hz, 1 H) 7.10 (t, J = 54.2 Hz, |
| | 1 H) 7.26-7.64 (m, 9 H) 7.93 (d, J = 8.8 Hz, 2 H) 8.16-8.33 (m, 1 H) |
| C630 | 1H NMR (400 MHz, DMSO-d₆, 81° C.) δ ppm 1.16 (d, J = 6.8 Hz, 3 H) |
| | 1.42 (d, J = 7.0 Hz, 3 H) 2.23-2.34 (m, 1 H) 2.48 (dt, J = 3.8, 1.9 Hz, 1 H) |
| | 2.81 (d, J = 4.6 Hz, 3 H) 3.95 (br d, J = 19.0 Hz, 1 H) 4.15-4.77 (m, 2 H) |
| | 5.27 (quin, J = 7.2 Hz, 1 H) 6.49 (br d, J = 7.6 Hz, 1 H) 6.81 (s, 1 H) 7.37 |
| | (dd, J = 8.2, 1.9 Hz, 1 H) 7.47-7.54 (m, 2 H) 7.60 (d, J = 8.1 Hz, 2 H) 7.64 |
| | (d, J = 1.9 Hz, 1 H) 7.65-7.68 (m, 1 H) 8.26 (br s, 1 H) 13.58 (br s, 1 H) |
| C631 | 1H NMR (400 MHz, DMSO-d₆) δ ppm 1.14-1.24 (m, 3 H) 1.32-1.45 (m, |
| | 3 H) 2.27-2.38 (m, 1 H) 2.52-2.53 (m, 1 H) 2.83-2.89 (m, 3 H) 3.90-4.11 (m, 1 H) |
| | 4.15-4.74 (m, 2 H) 5.21 (br t, J = 7.2 Hz, 1 H) 5.92 (br d, J = |
| | 7.5 Hz, 1 H) 7.19-7.35 (m, 4 H) 7.35-7.46 (m, 3 H) 7.65-7.68 (m, 1 H) |
| | 7.68-7.72 (m, 1 H) 7.97-8.07 (m, 2 H) 8.29-8.40 (m, 1 H) |
| C632 | 1H NMR (400 MHz, DMSO-d₆, 100° C.) δ ppm 1.10 (d, J = 6.8 Hz, 3 H) |
| | 1.52 (d, J = 7.0 Hz, 3 H) 1.55-1.71 (m, 4 H) 1.76-1.84 (m, 2 H) 2.15 (br |
| | t, J = 11.9 Hz, 1 H) 2.21-2.29 (m, 1 H) 2.36-2.46 (m, 1 H) 2.59 (d, J = |
| | 4.6 Hz, 5 H) 3.85 (br d, J = 18.7 Hz, 1 H) 4.29 (br t, J = 12.1 Hz, 2 H) 4.38 |
| | (br s, 1 H) 5.24 (quin, J = 7.0 Hz, 1 H) 6.95 (br d, J = 7.0 Hz, 1 H) 7.22 (d, |
| | J = 8.1 Hz, 2 H) 7.30-7.37 (m, 2 H) 7.45 (d, J = 7.9 Hz, 2 H) 7.59 (d, J = |
| | 1.8 Hz, 1 H) 7.63 (d, J = 8.1 Hz, 1 H) |
| C633 | 1H NMR (400 MHz, CHLOROFORM-D δ ppm 0.30 (dt, J = 9.9, 4.7 Hz, 1 |
| | H) 0.46-0.57 (m, 2 H) 0.66-0.77 (m, 1 H) 0.80-0.94 (m, 1 H) 1.20- |
| | 1.28 (m, 4 H) 2.48-2.56 (m, 1 H) 2.63-2.74 (m, 1 H) 3.04 (d, J = 4.8 Hz, |
| | 3 H) 3.94-4.05 (m, 1 H) 4.09-4.17 (m, 1 H) 4.22-5.13 (m, 1 H) 4.23- |
| | 4.36 (m, 1 H) 6.13-6.23 (m, 1 H) 7.19-7.24 (m, 1 H) 7.26-7.41 (m, 3 |
| | H) 7.49-7.55 (m, 2 H) 7.88-8.04 (m, 2 H) |
| C634 | 1H NMR (400 MHz, DMSO-d₆, 101° C.) δ ppm 1.07 (dd, J = 6.6, 3.3 Hz, 6 |
| | H) 1.21 (d, J = 6.8 Hz, 3 H) 2.36 (d, J = 16.5 Hz, 1 H) 2.53-2.62 (m, 1 H) |
| | 2.85 (d, J = 4.6 Hz, 3 H) 4.05 (br d, J = 18.8 Hz, 1 H) 4.14 (dq, J = 13.6, 6.8 |
| | Hz, 1 H) 4.54 (s, 2 H) 4.93 (br d, J = 7.7 Hz, 1 H) 7.19 (t, J = 54.3 Hz, 1 H) |
| | 7.34 (d, J = 8.3 Hz, 2 H) 7.44 (dd, J = 10.4, 9.1 Hz, 1 H) 7.69 (br d, J = 6.8 |
| | Hz, 2 H) 7.96-8.03 (m, 2 H) 8.25 (br d, J = 5.3 Hz, 1 H) |
| C635 | 1H NMR (400 MHz, DMSO-d₆, 101° C.) δ ppm 1.07 (dd, J = 6.6, 2.8 Hz, 6 |
| | H) 1.21 (d, J = 6.8 Hz, 3 H) 2.35 (d, J = 16.5 Hz, 1 H) 2.52-2.61 (m, 2 H) |
| | 2.85 (d, J = 4.6 Hz, 3 H) 4.03 (br d, J = 18.7 Hz, 1 H) 4.14 (dd, J = 13.6, 6.8 |
| | Hz, 1 H) 4.36-4.66 (m, 2 H) 4.94 (d, J = 7.7 Hz, 1 H) 7.34 (d, J = 8.2 Hz, 2 |
| | H) 7.51 (dd, J = 8.2, 1.9 Hz, 1 H) 7.57-7.60 (m, 1 H) 7.77 (d, J = 8.2 Hz, 1 |
| | H) 7.96-8.03 (m, 2 H) 8.25 (br d, J = 5.3 Hz, 1 H) |
| C636 | 1H NMR (400 MHz, DMSO-d₆, 100° C.) δ ppm 1.08-1.15 (m, 3 H) 1.38-1.46 (m, 1 H) |
| | 1.47-1.56 (m, 1 H) 1.57-1.68 (m, 5 H) 1.93 (br t, J = 12.7 |
| | Hz, 2 H) 2.25-2.32 (m, 1 H) 2.42-2.46 (m, 1 H) 2.50-2.63 (m, 2 H) |
| | 2.63-2.66 (m, 4 H) 3.88 (br d, J = 18.6 Hz, 1 H) 4.22-4.48 (m, 2 H) 5.28- |
| | 5.39 (m, 2 H) 7.22 (d, J = 8.2 Hz, 2 H) 7.30-7.37 (m, 2 H) 7.48-7.55 |
| | (m, 2 H) 7.60 (d, J = 1.9 Hz, 1 H) 7.64 (d, J = 8.3 Hz, 1 H) 7.79 (s, 1 H) |
| C637 | 1H NMR (400 MHz, DMSO-d₆, 101° C.) δ ppm 1.24 (d, J = 6.8 Hz, 3 H) |
| | 1.33 (dd, J = 6.8, 1.4 Hz, 6 H) 2.46 (d, J = 17.2 Hz, 1 H) 2.64-2.73 (m, 1 |
| | H) 2.86 (d, J = 4.6 Hz, 3 H) 3.88 (dt, J = 13.7, 6.8 Hz, 1 H) 4.19 (br d, J = |
| | 19.1 Hz, 1 H) 4.47-4.62 (m, 1 H) 4.66-4.84 (m, 1 H) 7.35-7.45 (m, 2 |
| | H) 7.47 (dd, J = 8.2, 2.0 Hz, 1 H) 7.72 (d, J = 1.9 Hz, 1 H) 7.74 (d, J = 8.2 |
| | Hz, 1 H) 7.96 (d, J = 8.8 Hz, 2 H) 8.27 (br d, J = 5.5 Hz, 1 H) |
| C638 | 1H NMR (400 MHz, DMSO-d₆, 100° C.) δ ppm 1.05 (dd, J = 6.5, 2.8 Hz, 6 |
| | H) 1.20 (d, J = 6.8 Hz, 3 H) 2.34 (d, J = 16.4 Hz, 1 H) 2.50-2.59 (m, 1 H) |
| | 2.90 (d, J = 4.8 Hz, 3 H) 4.03 (br d, J = 18.9 Hz, 1 H) 4.17 (dq, J = 13.6, 6.8 |
| | Hz, 1 H) 4.38-4.64 (m, 2 H) 5.33 (br d, J = 7.8 Hz, 1 H) 7.30 (d, J = 9.5 |
| | Hz, 1 H) 7.41 (dd, J = 8.4, 2.0 Hz, 1 H) 7.66 (d, J = 1.8 Hz, 1 H) 7.69 (d, J = |
| | 8.1 Hz, 1 H) 7.83 (s, 1 H) 8.20 (d, J = 8.4 Hz, 1 H) 8.67 (br d, J = 5.5 Hz, 1 |
| | H) |
| C639a | 1H NMR (400 MHz, DMSO-d₆, 81° C.) δ ppm 1.07 (dd, J = 6.6, 3.1 Hz, 6 |
| | H) 1.20 (d, J = 6.7 Hz, 3 H) 2.34 (br d, J = 16.3 Hz, 1 H) 2.58 (br dd, J = |
| | 16.4, 6.0 Hz, 1 H) 2.85 (d, J = 4.6 Hz, 2 H) 4.04 (br d, J = 18.8 Hz, 1 H) |
| | 4.21 (dq, J = 13.7, 6.9 Hz, 1 H) 4.31-4.83 (m, 2 H) 5.54 (d, J = 8.0 Hz, 1 |
| | H) 7.52 (d, J = 8.2 Hz, 1 H) 7.76-7.84 (m, 2 H) 7.89 (d, J = 1.7 Hz, 1 H) |
| | 7.94 (dd, J = 8.2, 1.9 Hz, 1 H) 8.09 (d, J = 1.9 Hz, 1 H) 8.47 (br d, J = 5.3 |
| | Hz, 1 H) |
| C639b | 1H NMR (400 MHz, DMSO-d₆, 101° C.) δ ppm 1.08 (dd, J = 6.6, 2.3 Hz, 6 |
| | H) 1.21 (d, J = 6.8 Hz, 3 H) 2.36 (d, J = 16.4 Hz, 1 H) 2.57 (br dd, J = 16.3, |
| | 6.1 Hz, 1 H) 2.86 (d, J = 4.6 Hz, 2 H) 4.07 (br d, J = 18.7 Hz, 1 H) 4.21 (dq, |
| | J = 13.7, 6.8 Hz, 1 H) 4.54 (s, 2 H) 5.40 (br d, J = 7.9 Hz, 1 H) 7.47 (d, J = |
| | 8.2 Hz, 1 H) 7.76 (dd, J = 8.4, 1.7 Hz, 1 H) 7.81 (d, J = 8.2 Hz, 1 H) 7.86 |
| | (d, J = 1.9 Hz, 1 H) 7.94 (dd, J = 8.2, 1.9 Hz, 1 H) 8.08 (d, J = 1.9 Hz, 1 H) |
| | 8.39 (br d, J = 5.3 Hz, 1 H) |
| C640 | 1H NMR (400 MHz, DMSO-d₆, 100° C.) δ ppm 1.09 (d, J = 6.8 Hz, 3 H) |
| | 1.54 (d, J = 7.0 Hz, 3 H) 1.56-1.65 (m, 2 H) 1.65-1.72 (m, 2 H) 1.76- |
| | 1.85 (m, 2 H) 2.09-2.20 (m, 1 H) 2.21-2.29 (m, 1 H) 2.37-2.46 (m, 1 |
| | H) 2.51-2.67 (m, 5 H) 3.83 (br d, J = 18.6 Hz, 1 H) 4.17-4.44 (m, 3 H) |
| | 5.26 (quin, J = 6.9 Hz, 1 H) 7.01 (br d, J = 6.8 Hz, 1 H) 7.31 (dd, J = 8.4, |
| | 2.0 Hz, 1 H) 7.34 (br d, J = 5.5 Hz, 1 H) 7.53-7.57 (m, 2 H) 7.58 (d, J = |
| | 2.0 Hz, 1 H) 7.61 (t, J = 7.9 Hz, 3 H) |
| C641 | 1H NMR (400 MHz, DMSO-d₆, 101° C.) δ ppm 1.03 (dd, J = 6.6, 3.1 Hz, 6 |
| | H) 1.17 (d, J = 6.8 Hz, 3 H) 2.31 (d, J = 16.4 Hz, 1 H) 2.55 (br dd, J = 16.3, |
| | 5.3 Hz, 1 H) 2.81 (d, J = 4.6 Hz, 3 H) 4.02 (br d, J = 18.7 Hz, 1 H) 4.11 (dq, |
| | J = 13.6, 6.7 Hz, 1 H) 4.32-4.58 (m, 2 H) 4.91 (br d, J = 7.7 Hz, 1 H) 7.30 |
| | (d, J = 8.2 Hz, 2 H) 7.54 (t, J = 9.0 Hz, 1 H) 7.81 (ddd, J = 8.7, 5.2, 2.2 Hz, |
| | 1 H) 7.92-8.00 (m, 3 H) 8.21 (br d, J = 5.5 Hz, 1 H) |
| C642 | 1H NMR (400 MHz, DMSO-d₆, 100° C.) δ ppm 1.08 (dd, J = 6.6, 3.5 Hz, 6 |
| | H) 1.19 (d, J = 6.6 Hz, 3 H) 2.28-2.46 (m, 1 H) 2.50-2.64 (m, 1 H) 2.80 |
| | (d, J = 4.6 Hz, 3 H) 4.02 (br d, J = 18.7 Hz, 1 H) 4.15 (dq, J = 13.6, 6.8 Hz, |
| | 1 H) 4.36-4.62 (m, 2 H) 5.25 (br d, J = 7.7 Hz, 1 H) 7.26 (dd, J = 8.1, 2.0 |
| | Hz, 1 H) 7.41 (s, 1 H) 7.57 (d, J = 8.1 Hz, 1 H) 7.71-7.76 (m, 1 H) 7.76-7.81 |
| | (m, 1 H) 7.83 (d, J = 2.0 Hz, 1 H) 8.01-8.14 (m, 1 H) |
| C643 | 1H NMR (400 MHz, DMSO-d₆, 100° C.) δ ppm 1.04 (dt, J = 6.5, 3.1 Hz, 6 |
| | H) 1.19 (dd, J = 6.7, 2.8 Hz, 3 H) 2.32 (br d, J = 16.1 Hz, 1 H) 2.50-2.60 |
| | (m, 2 H) 2.84 (d, J = 4.4 Hz, 3 H) 3.81 (d, J = 2.5 Hz, 3 H) 4.02 (br dd, J = |
| | 18.6, 10.7 Hz, 1 H) 4.08-4.20 (m, 1 H) 4.49 (br s, 2 H) 5.01 (br d, J = 7.6 |
| | Hz, 1 H) 7.23 (dd, J = 18.5, 8.0 Hz, 1 H) 7.54 (dd, J = 7.9, 1.8 Hz, 1 H) |
| | 7.63 (d, J = 1.5 Hz, 1 H) 7.72-7.81 (m, 2 H) 7.84 (d, J = 8.8 Hz, 1 H) 8.25 |
| | (br d, J = 5.1 Hz, 1 H) |
| C644 | 1H NMR (400 MHz, DMSO-d₆, 100° C.) δ ppm 1.11 (d, J = 6.8 Hz, 3 H) |
| | 1.40-1.47 (m, 1 H) 1.49-1.56 (m, 1 H) 1.59-1.69 (m, 5 H) 1.88-2.00 |
| | (m, 2 H) 2.24-2.32 (m, 1 H) 2.43 (br d, J = 5.3 Hz, 1 H) 2.56-2.72 (m, 1 |
| | H) 2.64 (d, J = 4.4 Hz, 3 H) 3.87 (br d, J = 18.7 Hz, 1 H) 4.23-4.34 (m, 1 |
| | H) 4.37 (br s, 1 H) 5.33 (br s, 1 H) 5.34-5.42 (m, 1 H) 7.33 (dd, J = 8.4, |
| | 2.0 Hz, 1 H) 7.40 (br d, J = 7.3 Hz, 1 H) 7.59 (d, J = 2.0 Hz, 1 H) 7.61 (s, 4 |
| | H) 7.64 (d, J = 8.4 Hz, 1 H) 7.79 (s, 1 H) |
| C645 | 1H NMR (400 MHz, DMSO-d₆, 81° C.) δ ppm 1.05-1.14 (m, 6 H) 1.14- |
| | 1.24 (m, 3 H) 2.25-2.38 (m, 1 H) 2.48-2.62 (m, 1 H) 2.78 (d, J = 4.8 Hz, |
| | 3 H) 3.51-3.63 (m, 3 H) 4.03 (br d, J = 18.8 Hz, 1 H) 4.17 (dq, J = 13.6, |
| | 6.8 Hz, 1 H) 4.25-4.94 (m, 2 H) 6.01-6.11 (m, 1 H) 6.62-6.72 (m, 1 H) |
| | 7.71-7.94 (m, 4 H) |
| C646 | 1H NMR (400 MHz, CHLOROFORM-d, 51° C.) δ ppm 1.00-1.19 (m, 6 H) |
| | 1.26 (d, J = 6.8 Hz, 3 H) 2.41-2.55 (m, 1 H) 2.61-2.75 (m, 1 H) 2.93- |
| | 3.04 (m, 3 H) 3.62-3.71 (m, 3 H) 3.86-5.55 (m, 5 H) 6.74-6.90 (m, 2 |
| | H) 7.52-7.77 (m, 4 H) |
| C643a | 1H NMR (400 MHz, DMSO-d₆, 100° C.) δ ppm 1.04 (t, J = 6.3 Hz, 6 H) |
| | 1.19 (d, J = 6.8 Hz, 3 H) 2.28-2.44 (m, 1 H) 2.50-2.65 (m, 1 H) 2.84 (d, |
| | J = 4.6 Hz, 3 H) 3.80 (s, 3 H) 4.01 (br d, J = 18.7 Hz, 1 H) 4.14 (dq, J = |
| | 13.7, 6.7 Hz, 1 H) 4.49 (br d, J = 25.6 Hz, 2 H) 5.00 (br d, J = 7.9 Hz, 1 H) |
| | 7.25 (d, J = 8.0 Hz, 1 H) 7.54 (dd, J = 8.1, 1.8 Hz, 1 H) 7.63 (d, J = 1.8 Hz, |
| | 1 H) 7.72-7.77 (m, 1 H) 7.77-7.81 (m, 1 H) 7.86 (d, J = 1.8 Hz, 1 H) |
| | 8.24 (br d, J = 5.0 Hz, 1 H) |
| C647a | 1H NMR (400 MHz, DMSO-d₆) δ ppm 1.13-1.21 (m, 3 H) 1.37-1.48 (m, |
| | 3 H) 2.26-2.35 (m, 1 H) 2.50-2.52 (m, 1 H) 2.80-2.86 (m, 3 H) 3.92-4.09 (m, 1 H) |
| | 4.18-4.72 (m, 2 H) 5.27-5.42 (m, 1 H) 5.58-5.75 (m, 1 |
| | H) 7.07-7.15 (m, 1 H) 7.21-7.52 (m, 5 H) 7.62-7.65 (m, 1 H) 7.65-7.69 (m, 1 H) |
| | 7.95-8.03 (m, 2 H) 8.05-8.13 (m, 1 H) 8.28-8.42 (m, 1 |
| | H) 11.82-12.31 (m, 1 H) |
| C647b | 1H NMR (400 MHz, DMSO-d₆) δ ppm 1.09-1.17 (m, 3 H) 1.37-1.46 (m, |
| | 3 H) 2.26-2.35 (m, 1 H) 2.50-2.51 (m, 1 H) 2.82 (d, J = 4.4 Hz, 3 H) |
| | 3.85-4.04 (m, 1 H) 4.13-4.86 (m, 2 H) 5.30-5.43 (m, 1 H) 5.59-5.71 |
| | (m, 1 H) 7.06-7.17 (m, 1 H) 7.32-7.52 (m, 4 H) 7.47 (br s, 1 H) 7.66- |
| | 7.69 (m, 1 H) 7.70 (s, 1 H) 7.96-8.03 (m, 2 H) 8.08 (s, 1 H) 8.28-8.38 |
| | (m, 1 H) 11.94-12.29 (m, 1 H) |
| C648 | 1H NMR (400 MHz, DMSO-d₆, 27° C.) δ ppm 0.92-0.99 (m, 2 H) 1.03-1.12 (m, 5 H) |
| | 1.12-1.26 (m, 1 H) 1.13-1.20 (m, 1 H) 2.14-2.48 (m, 1 |
| | H) 2.78-2.87 (m, 3 H) 3.57-3.96 (m, 2 H) 3.97-4.09 (m, 1 H) 4.09-4.17 (m, 1 H) |
| | 4.23 (dq, J = 13.6, 6.7 Hz, 1 H) 4.87-5.23 (m, 1 H) 5.40-5.57 (m, 1 H) 7.29-7.42 |
| | (m, 2 H) 7.53-7.70 (m, 1 H) 7.81-7.93 (m, 1 |
| | H) 7.94-8.03 (m, 3 H) 8.55 (br d, J = 5.0 Hz, 1 H) |
| C643b | 1H NMR (400 MHz, DMSO-d⁶, 100° C.) δ ppm 1.00-1.08 (m, 6 H) 1.16-1.22 (m, 3 H) |
| | 2.32 (br d, J = 16.3 Hz, 1 H) 2.50-2.58 (m, 1 H) 2.84 (d, J = |
| | 4.4 Hz, 3 H) 3.78-3.83 (m, 3 H) 3.97-4.08 (m, 1 H) 4.14 (dq, J = 13.6, |
| | 6.8 Hz, 1 H) 4.37-4.63 (m, 2 H) 5.01 (br d, J = 7.9 Hz, 1 H) 7.19-7.27 |
| | (m, 1 H) 7.54 (dd, J = 8.0, 1.7 Hz, 1 H) 7.63 (d, J = 1.8 Hz, 1 H) 7.74 (td, |
| | J = 7.4, 1.9 Hz, 1 H) 7.77-7.82 (m, 1 H) 7.82-7.88 (m, 1 H) 8.24 (br d, |
| | J = 5.3 Hz, 1 H) |
| C649 | 1H NMR (400 MHz, DMSO-d₆, 100° C.) δ ppm 1.07 (dd, J = 6.4, 3.3 Hz, 6 |
| | H) 1.20 (d, J = 6.8 Hz, 3 H) 2.33 (d, J = 16.4 Hz, 1 H) 2.50-2.59 (m, 1 H) |
| | 3.49-3.58 (m, 4 H) 3.60-3.69 (m, 4 H) 4.03 (br d, J = 18.8 Hz, 1 H) 4.12 |
| | (dq, J = 13.6, 6.7 Hz, 1 H) 4.38-4.64 (m, 2 H) 4.98 (d, J = 7.7 Hz, 1 H) |
| | 7.32 (d, J = 8.1 Hz, 2 H) 7.56 (d, J = 8.4 Hz, 2 H) 7.72-7.76 (m, 1 H) |
| | 7.76-7.81 (m, 1 H) 7.84 (d, J = 1.8 Hz, 1 H) |
| C650 | 1H NMR (400 MHz, DMSO-d₆, 100° C.) δ ppm 1.18 (d, J = 6.8 Hz, 3 H) |
| | 1.38 (d, J = 7.1 Hz, 3 H) 2.32 (d, J = 16.4 Hz, 1 H) 2.50-2.57 (m, 1 H) |
| | 2.84 (d, J = 4.6 Hz, 3 H) 3.99 (br d, J = 18.8 Hz, 1 H) 4.42 (br s, 2 H) 5.26 |
| | (quin, J = 7.1 Hz, 1 H) 5.82 (d, J = 7.5 Hz, 1 H) 7.37 (br d, J = 8.1 Hz, 2 H) |
| | 7.47 (d, J = 8.1 Hz, 2 H) 7.58 (d, J = 8.1 Hz, 2 H) 7.67-7.71 (m, 1 H) 7.72- |
| | 7.79 (m, 1 H) 7.81 (d, J = 2.0 Hz, 1 H) 7.97-8.04 (m, 2 H) 8.24 (br d, J = |
| | 5.0 Hz, 1 H) |
| C651 | 1H NMR (400 MHz, DMSO-d₆, 101° C.) δ ppm 1.03 (d, J = 6.8 Hz, 3 H) |
| | 1.20 (d, J = 7.0 Hz, 3 H) 2.16 (d, J = 16.3 Hz, 1 H) 2.35 (s, 1 H) 2.68 (d, J = |
| | 4.6 Hz, 3 H) 3.85 (br d, J = 18.8 Hz, 1 H) 4.28 (s, 2 H) 5.04 (quin, J = 7.1 |
| | Hz, 1 H) 5.46 (d, J = 7.7 Hz, 1 H) 6.87 (t, J = 8.8 Hz, 2 H) 7.12 (dd, J = 8.5, |
| | 5.5 Hz, 2 H) 7.19 (br d, J = 8.0 Hz, 2 H) 7.51-7.57 (m, 1 H) 7.58-7.63 |
| | (m, 1 H) 7.66 (d, J = 2.0 Hz, 1 H) 7.80-7.88 (m, 2 H) 8.08 (br d, J = 5.4 |
| | Hz, 1 H) |
| C652a | 1H NMR (400 MHz, DMSO-d₆) δ ppm 1.12-1.18 (m, 3 H) 1.30-1.36 (m, |
| | 3 H) 2.23-2.30 (m, 1 H) 2.50-2.51 (m, 1 H) 2.80-2.88 (m, 3 H) 3.85-4.03 (m, 1 H) |
| | 4.05-4.69 (m, 2 H) 5.47 (br t, J = 6.9 Hz, 1 H) 6.18 (d, J = |
| | 6.8 Hz, 1 H) 7.32-7.36 (m, 1 H) 7.36-7.48 (m, 2 H) 7.55-7.69 (m, 5 H) |
| | 7.99-8.07 (m, 2 H) 8.31-8.42 (m, 1 H) |
| C652b | 1H NMR (400 MHz, DMSO-d₆) δ ppm 1.06 (d, J = 6.8 Hz, 3 H) 1.30-1.38 |
| | (m, 3 H) 2.22-2.30 (m, 1 H) 2.49-2.50 (m, 1 H) 2.82-2.87 (m, 3 H) |
| | 3.71-3.89 (m, 1 H) 4.01-4.93 (m, 2 H) 5.44-5.64 (m, 1 H) 6.09-6.21 |
| | (m, 1 H) 7.36-7.43 (m, 3 H) 7.57-7.76 (m, 5 H) 7.98-8.07 (m, 2 H) |
| | 8.29-8.46 (m, 1 H) |
| C653 | 1H NMR (400 MHz, DMSO-d₆, 81° C.) δ ppm 1.15-1.26 (m, 3 H) 2.28 (br |
| | d, J = 16.6 Hz, 1 H) 2.50-2.62 (m, 1 H) 2.76 (d, J = 4.8 Hz, 3 H) 3.54-3.67 |
| | (m, 3 H) 4.01 (br d, J = 19.1 Hz, 1 H) 4.24-4.92 (m, 2 H) 6.51-6.61 |
| | (m, 1 H) 7.70-7.92 (m, 4 H) 9.63 (br s, 1 H) |
| C654 | 1H NMR (400 MHz, DMSO-d₆, 100° C.) δ ppm 0.56-0.64 (m, 2 H) 0.68-0.76 (m, 2 H) |
| | 1.01-1.07 (m, 6 H) 1.20 (dd, J = 6.7, 1.4 Hz, 3 H) 2.30-2.38 (m, 1 H) 2.51-2.60 |
| | (m, 1 H) 2.89 (td, J = 7.5, 3.7 Hz, 1 H) 4.03 (br d, |
| | J = 18.7 Hz, 1 H) 4.13 (dq, J = 13.5, 6.8 Hz, 1 H) 4.37-4.64 (m, 2 H) 4.93 |
| | (br d, J = 7.8 Hz, 1 H) 7.31 (d, J = 8.0 Hz, 2 H) 7.72-7.76 (m, 1 H) 7.76-7.82 |
| | (m, 1 H) 7.84 (s, 1 H) 7.97 (d, J = 7.9 Hz, 2 H) 8.25 (br d, J = 4.2 Hz, |
| | 1 H) |
| C655 | 1H NMR (400 MHz, CHLOROFORM-D δ ppm 1.23 (br d, J = 6.8 Hz, 4 H) |
| | 1.39-1.47 (m, 5 H) 2.46-2.54 (m, 1 H) 2.62 (br d, J = 5.5 Hz, 1 H) 3.06 |
| | (d, J = 4.8 Hz, 3 H) 3.98 (br d, J = 18.9 Hz, 1 H) 4.36-4.90 (m, 2 H) 5.20-5.30 |
| | (m, 1 H) 5.62 (d, J = 6.6 Hz, 1 H) 6.23 (br d, J = 3.7 Hz, 1 H) 7.21 (dd, |
| | J = 8.3, 1.9 Hz, 1 H) 7.30-7.37 (m, 2 H) 7.45-7.51 (m, 2 H) 7.52-7.59 |
| | (m, 3 H) 8.25 (dd, J = 8.3, 2.3 Hz, 1 H) 8.99 (d, J = 2.0 Hz, 1 H) |
| C656 | 1H NMR (400 MHz, DMSO-d₆, 126° C.) δ ppm 1.15 (dd, J = 6.6, 3.1 Hz, 7 |
| | H) 1.24 (d, J = 6.8 Hz, 4 H) 2.35-2.42 (m, 2 H) 2.57 (s, 3 H) 4.06 (br d, |
| | J = 18.9 Hz, 1 H) 4.17-4.28 (m, 1 H) 4.49-4.67 (m, 2 H) 0.00 (br d, J = |
| | 8.1 Hz, 1 H) 7.39 (dd, J = 5.2, 1.7 Hz, 1 H) 7.45 (dd, J = 8.2, 1.9 Hz, 1 H) |
| | 7.54 (dd, J = 1.8, 0.4 Hz, 1 H) 7.69 (d, J = 1.9 Hz, 1 H) 7.73 (d, J = 8.2 Hz, |
| | 1 H) 8.62 (dd, J = 5.2, 0.5 Hz, 1 H) |
| C657 | 1H NMR (400 MHz, DMSO-d₆) δ ppm 0.77 (s, 6 H) 1.14-1.21 (m, 3 H) |
| | 2.28-2.40 (m, 1 H) 2.52-2.61 (m, 1 H) 2.81 (d, J = 4.6 Hz, 3 H) 2.91- |
| | 3.03 (m, 4 H) 4.07 (br d, J = 19.1 Hz, 1 H) 4.56 (td, J = 5.7, 2.6 Hz, 2 H) |
| | 7.35-7.41 (m, 2 H) 7.41-7.47 (m, 1 H) 7.67-7.69 (m, 1 H) 7.69-7.73 |
| | (m, 1 H) 7.92 (d, J = 8.6 Hz, 2 H) 8.22-8.37 (m, 1 H) |
| C658 | 1H NMR (400 MHz, DMSO-d₆) δ ppm 1.13-1.22 (m, 3 H) 1.40 (br d, J = |
| | 6.8 Hz, 3 H) 2.24-2.37 (m, 1 H) 2.49-2.59 (m, 1 H) 2.75-2.82 (m, 3 H) |
| | 3.42-3.68 (m, 3 H) 3.90-4.04 (m, 1 H) 4.26-4.61 (m, 2 H) 5.07-5.32 |
| | (m, 1 H) 6.49-6.80 (m, 2 H) 6.97-7.11 (m, 2 H) 7.27-7.40 (m, 2 H) |
| | 7.66-7.85 (m, 4 H) |
| C659a | 1H NMR (400 MHz, DMSO-d₆) δ ppm 1.13-1.21 (m, 3 H) 1.32-1.44 (m, |
| | 3 H) 1.88-1.91 (m, 1 H) 2.02-2.04 (m, 1 H) 2.25-2.37 (m, 1 H) 2.50-2.56 (m, 1 H) |
| | 2.81-2.88 (m, 3 H) 3.85-4.04 (m, 1 H) 4.09-4.86 (m, 2 |
| | H) 5.19-5.32 (m, 1 H) 5.96-6.12 (m, 1 H) 7.12-7.48 (m, 2 H) 7.20- |
| | 7.49 (m, 1 H) 7.26-7.47 (m, 2 H) 7.60-7.70 (m, 3 H) 8.01 (d, J = 8.8 Hz, |
| | 2 H) 8.26-8.38 (m, 1 H) |
| C659b | 1H NMR (400 MHz, DMSO-d₆) δ ppm 1.08-1.16 (m, 3 H) 1.32-1.41 (m, |
| | 3 H) 2.25-2.35 (m, 1 H) 2.51-2.58 (m, 1 H) 2.80-2.86 (m, 3 H) 3.90 (br |
| | d, J = 17.4 Hz, 1 H) 4.09-4.83 (m, 2 H) 5.31 (t, J = 7.3 Hz, 1 H) 5.98-6.10 (m, 1 H) |
| | 7.40 (br dd, J = 8.3, 1.9 Hz, 5 H) 7.29-7.44 (m, 1 H) 7.62-7.66 (m, 1 H) 7.66-7.68 |
| | (m, 1 H) 7.68-7.72 (m, 1 H) 8.01 (d, J = 8.8 Hz, |
| | 2 H) 8.25-8.41 (m, 1 H) |
| C660a | 1H NMR (400 MHz, DMSO-d₆) δ ppm 1.08-1.16 (m, 3 H) 1.33-1.42 (m, |
| | 3 H) 2.26-2.34 (m, 1 H) 2.51-2.58 (m, 1 H) 2.80-2.87 (m, 3 H) 3.91 (br |
| | d, J = 19.4 Hz, 1 H) 4.14-4.84 (m, 2 H) 5.24-5.35 (m, 1 H) 5.92 (d, J = |
| | 7.9 Hz, 1 H) 6.77-7.09 (m, 1 H) 7.29-7.63 m, 2 H) 7.34-7.52 (m, 6 H) |
| | 7.66-7.68 (m, 1 H) 7.68-7.72 (m, 1 H) 7.95-8.04 (m, 2 H) 8.29-8.39 |
| | (m, 1 H) |
| C660b | 1H NMR (400 MHz, DMSO-d₆) δ ppm 1.13-1.20 (m, 3 H) 1.34-1.40 (m, |
| | 3 H) 2.26-2.34 (m, 1 H) 2.50-2.55 (m, 1 H) 2.79-2.88 (m, 3 H) 3.90-4.11 (m, 1 H) |
| | 4.18-4.78 (m, 2 H) 5.26 (s, 1 H) 5.88-6.02 (m, 1 H) 6.72-7.07 (m, 1 H) 7.23-7.52 |
| | (m, 7 H) 7.63-7.65 (m, 1 H) 7.65-7.69 (m, 1 |
| | H) 7.95-8.05 (m, 2 H) 8.28-8.40 (m, 1 H) |
| C661 | 1H NMR (400 MHz, DMSO-d⁶, 100° C.) δ ppm 0.62-0.86 (m, 4 H) 1.01-1.08 (m, 6 H) |
| | 1.16-1.23 (m, 3 H) 1.50-1.60 (m, 1 H) 2.31-2.45 (m, 1 |
| | H) 2.50-2.66 (m, 1 H) 2.82 (d, J = 4.6 Hz, 3 H) 4.04 (br d, J = 18.7 Hz, 1 |
| | H) 4.17 (dq, J = 13.6, 6.8 Hz, 1 H) 4.38-4.65 (m, 2 H) 4.81-4.96 (m, 1 |
| | H) 7.17-7.24 (m, 1 H) 7.54-7.62 (m, 1 H) 7.72-7.76 (m, 1 H) 7.76-7.80 (m, 2 H) |
| | 7.84 (d, J = 1.8 Hz, 1 H) 8.21 (q, J = 4.5 Hz, 1 H) |
| C662 | 1H NMR (400 MHz, DMSO-d₆, 100° C.) δ ppm 1.07-1.22 (m, 7 H) 2.30 |
| | (d, J = 16.5 Hz, 1 H) 2.47-2.57 (m, 1 H) 2.83 (d, J = 4.6 Hz, 3 H) 3.89 (br |
| | d, J = 18.8 Hz, 1 H) 4.26-4.60 (m, 2 H) 6.42 (s, 1 H) 7.06-7.15 (m, 1 H) |
| | 7.17-7.26 (m, 4 H) 7.31-7.41 (m, 3 H) 7.62 (d, J = 1.9 Hz, 1 H) 7.66 (d, |
| | J = 8.2 Hz, 1 H) 7.98 (d, J = 8.6 Hz, 2 H) 8.16-8.27 (m, 1 H) |
| C661a | 1H NMR (400 MHz, DMSO-d₆, 100° C.) δ ppm 0.61-0.87 (m, 4 H) 1.05 |
| | (d, J = 6.6 Hz, 6 H) 1.20 (d, J = 6.8 Hz, 3 H) 1.50-1.58 (m, 1 H) 2.29-2.46 (m, 1 H) |
| | 2.50-2.65 (m, 1 H) 2.82 (d, J = 4.6 Hz, 3 H) 4.04 (br d, J = |
| | 18.7 Hz, 1 H) 4.17 (dq, J = 13.6, 6.8 Hz, 1 H) 4.51 (br d, J = 38.4 Hz, 2 H) |
| | 4.87 (br d, J = 7.9 Hz, 1 H) 7.22 (d, J = 8.1 Hz, 1 H) 7.55 (d, J = 2.0 Hz, 1 |
| | H) 7.72-7.81 (m, 3 H) 7.84 (d, J = 1.8 Hz, 1 H) 8.21 (br d, J = 5.3 Hz, 1 H) |
| C661b | 1H NMR (400 MHz, DMSO-d₆, 100° C.) δ ppm 0.61-0.86 (m, 4 H) 1.05 |
| | (dd, J = 6.5, 3.4 Hz, 6H) 1.18 (d, J = 6.8 Hz, 3 H) 1.52-1.59 (m, 1 H) 2.36 |
| | (d, J = 16.3 Hz, 1 H) 2.50-2.65 (m, 1 H) 2.82 (d, J = 4.6 Hz, 3 H) 4.04 (br |
| | d, J = 18.9 Hz, 1 H) 4.06-4.13 (m, 1 H) 4.17 (dq, J = 13.6, 6.8 Hz, 1 H) |
| | 4.51 (br d, J = 27.1 Hz, 2 H) 4.91 (d, J = 7.9 Hz, 1 H) 7.19 (d, J = 8.1 Hz, 1 |
| | H) 7.59 (d, J = 2.0 Hz, 1 H) 7.72-7.76 (m, 1 H) 7.76-7.82 (m, 2 H) 7.84 |
| | (d, J = 2.0 Hz, 1 H) 8.22 (br d, J = 5.5 Hz, 1 H) |
| C663a | 1H NMR (400 MHz, DMSO-d₆) δ ppm 1.12-1.20 (m, 1 H) 1.36 (d, J = 7.0 |
| | Hz, 3 H) 2.25-2.34 (m, 1 H) 2.50-2.54 (m, 1 H) 2.84 (d, J = 4.6 Hz, 3 H) |
| | 3.06 (s, 28 H) 3.90-4.03 (m, 1 H) 4.11-4.66 (m, 2 H) 5.38-5.47 (m, 1 |
| | H) 5.97 (d, J = 7.3 Hz, 1 H) 7.30-7.48 (m, 5 H) 7.32-7.58 (m, 6 H) 7.63 |
| | (d, J = 1.8 Hz, 1 H) 7.66 (d, J = 8.1 Hz, 1 H) 8.02 (d, J = 8.0 Hz, 2 H) 8.31-8.38 (m, 1 H) |
| C663b | 1H NMR (400 MHz, DMSO-d₆) δ ppm 1.09 (d, J = 6.8 Hz, 3 H) 1.33-1.40 |
| | (m, 3 H) 2.28 (d, J = 16.7 Hz, 1 H) 2.51-2.59 (m, 1 H) 2.84 (d, J = 4.6 Hz, |
| | 3 H) 3.06 (s, 25 H) 3.85 (br d, J = 17.6 Hz, 1 H) 4.12-4.84 (m, 2 H) 5.47 |
| | (quin, J = 7.1 Hz, 1 H) 5.96 (d, J = 7.5 Hz, 1 H) 7.37-7.43 (m, 3 H) 7.47-7.61 |
| | (m, 3 H) 7.66 (d, J = 2.0 Hz, 1 H) 7.70 (d, J = 8.1 Hz, 1 H) 8.02 (d, J = |
| | 7.8 Hz, 2 H) 8.30-8.40 (m, 1 H) |
| C664a | 1H NMR (400 MHz, DMSO-d₆) δ ppm 1.14-1.20 (m, 3 H) 1.33-1.41 (m, |
| | 3 H) 2.26-2.36 (m, 1 H) 2.50-2.59 (m, 1 H) 2.84 (d, J = 4.6 Hz, 3 H) |
| | 3.07 (s, 31 H) 3.85-4.08 (m, 1 H) 4.14-4.81 (m, 2 H) 5.19-5.28 (m, 1 |
| | H) 6.11 (br s, 1 H) 7.31-7.48 (m, 3 H) 7.42-7.46 (m, 1 H) 7.58 (s, 1 H) |
| | 7.64 (d, J = 1.8 Hz, 1 H) 7.67 (d, J = 8.4 Hz, 1 H) 7.69-7.74 (m, 1 H) 8.01 |
| | (d, J = 7.8 Hz, 2 H) 8.30-8.38 (m, 1 H) |
| C664b | 1H NMR (400 MHz, DMSO-d₆) δ ppm 1.12 (d, J = 6.8 Hz, 3 H) 1.33-1.43 |
| | (m, 3 H) 2.27-2.35 (m, 1 H) 2.51-2.58 (m, 1 H) 2.83 (d, J = 4.6 Hz, 3 H) |
| | 3.07 (s, 43 H) 3.91 (br d, J = 19.1 Hz, 1 H) 4.82 (s, 2 H) 5.25-5.34 (m, 1 |
| | H) 6.06-6.13 (m, 1 H) 7.36-7.43 (m, 3 H) 7.44-7.49 (m, 1 H) 7.60-7.63 (m, 1 H) |
| | 7.65-7.68 (m, 1 H) 7.68-7.72 (m, 1 H) 7.72-7.76 (m, 1 |
| | H) 8.01 (d, J = 8.8 Hz, 2 H) 8.34 (br d, J = 4.4 Hz, 1 H) |
| C665a | 1H NMR (400 MHz, DMSO-d₆, 101° C.) δ ppm 1.22 (d, J = 6.8 Hz, 3 H) |
| | 1.43 (d, J = 6.9 Hz, 3 H) 2.36 (br d, J = 16.7 Hz, 1 H) 2.52-2.62 (m, 2 H) |
| | 2.86 (d, J = 4.6 Hz, 3 H) 4.03 (br d, J = 18.8 Hz, 1 H) 4.33-4.60 (m, 2 H) |
| | 5.31 (quin, J = 7.0 Hz, 1 H) 5.86 (br d, J = 7.3 Hz, 1 H) 7.43 (br d, J = 8.0 |
| | Hz, 2 H) 7.73 (dd, J = 8.1, 1.8 Hz, 1 H) 7.79 (d, J = 8.1 Hz, 1 H) 7.83 (d, J = |
| | 1.9 Hz, 1 H) 8.03 (d, J = 8.9 Hz, 2 H) 8.27 (br d, J = 5.4 Hz, 1 H) 8.41- |
| | 8.49 (m, 2 H) 8.64 (d, J = 1.5 Hz, 1 H) |
| C665b | 1H NMR (400 MHz, DMSO-d₆, 101° C.) δ ppm 1.17 (d, J = 6.8 Hz, 3 H) |
| | 1.43 (d, J = 6.9 Hz, 3 H) 2.36 (d, J = 16.6 Hz, 1 H) 2.59 (br dd, J = 16.4, 6.2 |
| | Hz, 1 H) 2.86 (d, J = 4.6 Hz, 3 H) 3.97 (br d, J = 18.8 Hz, 1 H) 4.33-4.68 |
| | (m, 2 H) 5.34 (quin, J = 7.0 Hz, 1 H) 5.88 (d, J = 7.4 Hz, 1 H) 7.43 (d, J = |
| | 8.5 Hz, 2 H) 7.75 (dd, J = 8.4, 1.8 Hz, 1 H) 7.81 (d, J = 8.4 Hz, 1 H) 7.86 |
| | (d, J = 1.9 Hz, 1 H) 8.03 (d, J = 8.6 Hz, 2 H) 8.28 (br d, J = 5.4 Hz, 1 H) |
| | 8.42-8.50 (m, 2 H) 8.66 (d, J = 1.5 Hz, 1 H) |
| C666 | 1H NMR (400 MHz, DMSO-d₆, 101° C.) δ ppm 1.12-1.20 (m, 9 H) 1.31-1.44 (m, 6 H) |
| | 2.34-2.43 (m, 1 H) 2.55-2.61 (m, 1 H) 4.02 (br d, J = 18.8 |
| | Hz, 1 H) 4.21 (q, J = 7.0 Hz, 2 H) 4.36 (q, J = 7.0 Hz, 2 H) 4.49-4.63 (m, 2 |
| | H) 4.80-4.92 (m, 1 H) 6.49 (d, J = 1.8 Hz, 1 H) 6.70 (dd, J = 5.6, 1.8 Hz, 1 |
| | H) 7.19 (d, J = 8.5 Hz, 1 H) 7.36 (dd, J = 8.4, 2.1 Hz, 1 H) 7.47 (d, J = 2.1 |
| | Hz, 1 H) 8.12 (d, J = 5.5 Hz, 1 H) |
| C667 | 1H NMR (400 MHz, DMSO-d₆, 101° C.) δ ppm 1.18 (dt, J = 6.7, 1.5 Hz, 9 |
| | H) 1.35 (t, J = 7.0 Hz, 3 H) 2.34-2.43 (m, 1 H) 2.53-2.63 (m, 1 H) 4.03 |
| | (br d, J = 18.7 Hz, 1 H) 4.37 (q, J = 7.0 Hz, 2 H) 4.44-4.64 (m, 2 H) 4.85 |
| | (spt, J = 6.8 Hz, 1 H) 6.49 (d, J = 1.9 Hz, 1 H) 6.70 (dd, J = 5.5, 1.9 Hz, 1 |
| | H) 7.41 (dd, J = 8.2, 2.0 Hz, 1 H) 7.65 (d, J = 1.9 Hz, 1 H) 7.69 (d, J = 8.2 |
| | Hz, 1 H) 8.13 (d, J = 5.5 Hz, 1 H) 10.36-10.80 (m, 1 H) |
| C668 | 1H NMR (400 MHz, DMSO-d₆, 100° C.) δ ppm 1.04-1.13 (m, 6 H) 1.18 |
| | (d, J = 6.8 Hz, 3 H) 2.32 (d, J = 16.2 Hz, 1 H) 2.51-2.59 (m, 1 H) 2.78 (d, |
| | J = 4.6 Hz, 3 H) 4.01 (br d, J = 18.8 Hz, 1 H) 4.07-4.19 (m, 4 H) 4.34- |
| | 4.70 (m, 2 H) 5.64 (d, J = 7.7 Hz, 1 H) 6.79 (s, 1 H) 7.70-7.76 (m, 1 H) |
| | 7.76-7.81 (m, 1 H) 7.84 (d, J = 1.9 Hz, 1 H) 8.16-8.26 (m, 1 H) |
| C669a | 1H NMR (400 MHz, DMSO-d₆, 100° C.) δ ppm 1.04 (t, J = 6.6 Hz, 6 H) |
| | 1.20 (d, J = 6.8 Hz, 3 H) 2.34 (d, J = 16.3 Hz, 1 H) 2.50-2.59 (m, 1 H) |
| | 2.85 (d, J = 4.6 Hz, 3 H) 4.04 (br d, J = 18.7 Hz, 1 H) 4.18 (dq, J = 13.8, 6.8 |
| | Hz, 1 H) 4.50 (br d, J = 28.1 Hz, 2 H) 5.21 (d, J = 7.9 Hz, 1 H) 6.67 (t, J = |
| | 54.6 Hz, 1 H) 7.40 (d, J = 8.2 Hz, 1 H) 7.71-7.76 (m, 1 H) 7.77-7.81 (m, |
| | 1 H) 7.84 (d, J = 2.0 Hz, 1 H) 8.12 (d, J = 8.4 Hz, 1 H) 8.21 (s, 1 H) 8.42 |
| | (br d, J = 5.6 Hz, 1 H) |
| C669b | 1H NMR (400 MHz, DMSO-d₆, 100° C.) δ ppm 1.04 (dd, J = 12.5, 6.6 Hz, |
| | 6 H) 1.20 (d, J = 6.6 Hz, 3 H) 2.33 (d, J = 16.4 Hz, 1 H) 2.50-2.66 (m, 1 |
| | H) 2.85 (d, J = 4.6 Hz, 3 H) 4.03 (br d, J = 18.9 Hz, 1 H) 4.18 (dq, J = 13.7, |
| | 6.7 Hz, 1 H) 4.51 (br d, J = 39.5 Hz, 2 H) 5.22 (d, J = 7.9 Hz, 1 H) 6.49-6.82 |
| | (m, 1 H) 7.44 (d, J = 8.2 Hz, 1 H) 7.73-7.77 (m, 1 H) 7.77-7.81 (m, |
| | 1 H) 7.85 (d, J = 2.0 Hz, 1 H) 8.13 (d, J = 8.1 Hz, 1 H) 8.21 (s, 1 H) 8.44 |
| | (br d, J = 5.4 Hz, 1 H) |
| C670 | 1H NMR (400 MHz, DMSO-d₆) δ ppm 1.00-1.09 (m, 3 H) 1.18 (d, J = 6.8 |
| | Hz, 3 H) 1.20-1.28 (m, 2 H) 1.31-1.49 (m, 4 H) 2.27-2.40 (m, 1 H) |
| | 2.50-2.57 (m, 1 H) 3.32-3.39 (m, 2 H) 3.79-3.86 (m, 6 H) 3.95-4.10 |
| | (m, 3 H) 4.35-4.62 (m, 1 H) 5.23 (dd, J = 8.0, 5.4 Hz, 1 H) 6.89 (br d, J = |
| | 7.3 Hz, 1 H) 7.08 (s, 1 H) 7.41 (d, J = 8.4 Hz, 1 H) 7.67 (d, J = 2.0 Hz, 1 H) |
| | 7.70 (dd, J = 8.1, 0.9 Hz, 1 H) 7.76 (d, J = 8.1 Hz, 1 H) |
| C671 | 1H NMR (400 MHz, DMSO-d₆, 100° C.) δ ppm 1.05-1.13 (m, 6 H) 1.15-1.23 (m, 3 H) |
| | 2.27-2.38 (m, 1 H) 2.51-2.63 (m, 1 H) 2.81 (d, J = 4.9 Hz, |
| | 3 H) 3.57-3.64 (m, 3 H) 4.02 (br d, J = 18.9 Hz, 1 H) 4.16 (dq, J = 13.6, |
| | 6.8 Hz, 1 H) 4.29-4.76 (m, 2 H) 5.92-6.05 (m, 1 H) 6.98-7.11 (m, 1 H) |
| | 7.70-7.87 (m, 3 H) 8.10-8.23 (m, 1 H) |
| C672 | 1H NMR (400 MHz, DMSO-d₆, 100° C.) δ ppm 1.02-1.14 (m, 6 H) 1.15-1.23 (m, 3 H) |
| | 2.28-2.39 (m, 1 H) 2.50-2.62 (m, 1 H) 2.79 (d, J = 4.8 Hz, |
| | 3 H) 3.46-3.65 (m, 3 H) 4.02 (br d, J = 18.9 Hz, 1 H) 4.16 (dq, J = 13.5, |
| | 6.7 Hz, 1 H) 4.29-4.77 (m, 2 H) 5.84-5.97 (m, 1 H) 6.62-6.73 (m, 1 H) |
| | 7.17 (t, J = 54.2 Hz, 1 H) 7.57-7.72 (m, 3 H) 7.73-7.85 (m, 1 H) |
| C673 | 1H NMR (400 MHz, DMSO-d₆, 100° C.) δ ppm 0.56-0.64 (m, 2 H) 0.69-0.78 (m, 2 H) |
| | 1.03-1.09 (m, 7 H) 1.15-1.22 (m, 3 H) 2.29-2.39 (m, 1 |
| | H) 2.51-2.62 (m, 1 H) 2.90 (tq, J = 7.5, 3.9 Hz, 1 H) 3.98-4.09 (m, 1 H) |
| | 4.12-4.24 (m, 1 H) 4.50 (br d, J = 22.3 Hz, 2 H) 5.36 (d, J = 7.9 Hz, 1 H) |
| | 7.41-7.50 (m, 1 H) 7.72-7.77 (m, 1 H) 7.77-7.81 (m, 1 H) 7.82-7.87 |
| | (m, 1 H) 7.92 (dd, J = 8.1, 1.8 Hz, 1 H) 8.06 (d, J = 1.9 Hz, 1 H) 8.37 (br d, |
| | J = 4.2 Hz, 1 H) |
| C674 | 1H NMR (400 MHz, DMSO-d₆, 126° C.) δ ppm 1.09 (dd, J = 6.5, 2.0 Hz, 6 |
| | H) 1.23 (d, J = 6.8 Hz, 4 H) 2.38 (br d, J = 16.4 Hz, 1 H) 2.55-2.64 (m, 1 |
| | H) 2.86 (d, J = 4.6 Hz, 3 H) 4.05 (br d, J = 19.2 Hz, 1 H) 4.15 (dq, J = 13.5, |
| | 6.8 Hz, 1 H) 4.51-4.75 (m, 1 H) 4.78 (br d, J = 6.8 Hz, 1 H) 7.34 (d, J = |
| | 8.4 Hz, 2 H) 7.88 (s, 1 H) 7.99 (d, J = 8.6 Hz, 2 H) 8.13 (br d, J = 3.9 Hz, 1 |
| | H) 8.77 (s, 1 H) |
| C675 | 1H NMR (400 MHz, DMSO-d₆, 101° C.) δ ppm 1.07 (br s, 6 H) 1.30 (d, J = |
| | 6.8 Hz, 3 H) 2.34-2.45 (m, 1 H) 2.60-2.74 (m, 1 H) 2.85 (d, J = 4.6 Hz, 3 |
| | H) 3.93-4.32 (m, 1 H) 4.35-4.82 (m, 1 H) 4.95 (br d, J = 8.1 Hz, 2 H) |
| | 7.36 (d, J = 8.1 Hz, 2 H) 7.90-7.98 (m, 2 H) 8.00 (d, J = 8.8 Hz, 2 H) 8.10 |
| | -8.20 (m, 2 H) 8.25 (br d, J = 5.0 Hz, 1 H) 9.12 (s, 1 H) |
| C676a | 1H NMR (400 MHz, DMSO-d₆, 126° C.) δ ppm 0.10-0.40 (m, 4 H) 0.81-0.95 (m, 1 H) |
| | 1.12 (d, J = 6.7 Hz, 3 H) 1.18 (d, J = 6.8 Hz, 3 H) 2.33 (br d, |
| | J = 16.3 Hz, 1 H) 2.52-2.61 (m, 1 H) 2.83 (d, J = 4.6 Hz, 3 H) 3.57 (br d, |
| | J = 7.4 Hz, 1 H) 3.98 (br d, J = 18.8 Hz, 1 H) 4.36-4.48 (m, 1 H) 4.52 (s, 1 |
| | H) 5.12-5.23 (m, 1 H) 7.48 (d, J = 8.2 Hz, 1 H) 7.68-7.77 (m, 2 H) 7.82 |
| | (s, 1 H) 7.92 (dd, J = 8.2, 1.9 Hz, 1 H) 8.06 (d, J = 1.9 Hz, 1 H) 8.25 (br d, |
| | J = 4.3 Hz, 1 H) |
| C676b | 1H NMR (400 MHz, DMSO-d₆, 101° C.) δ ppm 0.17 (dt, J = 9.3, 4.6 Hz, 1 |
| | H) 0.21-0.28 (m, 1 H) 0.29-0.35 (m, 1 H) 0.36-0.45 (m, 1 H) 0.84-0.99 (m, 1 H) |
| | 1.15 (d, J = 6.7 Hz, 3 H) 1.20 (d, J = 6.8 Hz, 3 H) 2.36 (br d, |
| | J = 16.5 Hz, 1 H) 2.53-2.62 (m, 1 H) 2.86 (d, J = 4.6 Hz, 3 H) 3.48-3.62 |
| | (m, 1 H) 4.03 (br d, J = 18.7 Hz, 1 H) 4.35-4.66 (m, 2 H) 5.47 (br d, J = |
| | 7.8 Hz, 1 H) 7.49 (d, J = 8.2 Hz, 1 H) 7.75 (dd, J = 8.1, 1.5 Hz, 1 H) 7.81 |
| | (d, J = 8.1 Hz, 1 H) 7.85 (d, J = 1.9 Hz, 1 H) 7.96 (dd, J = 8.2, 1.9 Hz, 1 H) |
| | 8.10 (d, J = 1.9 Hz, 1 H) 8.40 (br d, J = 5.3 Hz, 1 H) |
| C677b | 1H NMR (400 MHz, DMSO-d₆, 101° C.) δ ppm 1.21 (d, J = 6.8 Hz, 4 H) |
| | 1.30-1.37 (m, 10 H) 2.37 (d, J = 16.4 Hz, 1 H) 2.55-2.64 (m, 1 H) 2.85 |
| | (d, J = 4.6 Hz, 3 H) 4.08 (br d, J = 18.9 Hz, 1 H) 4.29 (s, 1 H) 4.37-4.53 |
| | (m, 1 H) 4.57-4.73 (m, 1 H) 7.55 (d, J = 8.2 Hz, 1 H) 7.76 (dd, J = 8.4, 1.8 |
| | Hz, 1 H) 7.81 (d, J = 8.4 Hz, 1 H) 7.86 (d, J = 1.9 Hz, 1 H) 7.96 (dd, J = 8.2, |
| | 1.9 Hz, 1 H) 8.11 (d, J = 1.9 Hz, 1 H) 8.41 (s, 1 H) |
| C678 | 1H NMR (400 MHz, DMSO-d₆, 100° C.) δ ppm 0.58-0.67 (m, 2 H) 0.67-0.75 (m, 2 H) |
| | 1.00-1.07 (m, 7 H) 1.14-1.22 (m, 3 H) 2.23-2.44 (m, 1 |
| | H) 2.50-2.58 (m, 1 H) 2.88 (tt, J = 7.4, 3.9 Hz, 1 H) 3.76-3.84 (m, 3 H) |
| | 3.95-4.07 (m, 1 H) 4.07-4.19 (m, 1 H) 4.36-4.62 (m, 2 H) 4.99 (br d, |
| | J = 7.9 Hz, 1 H) 7.17-7.27 (m, 1 H) 7.49-7.56 (m, 1 H) 7.56-7.63 (m, 1 |
| | H) 7.71-7.81 (m, 2 H) 7.84 (br d, J = 8.8 Hz, 1 H) 8.24 (s, 1 H) |
| C679 | 1H NMR (400 MHz, DMSO-d₆, 101° C.) δ ppm 1.08 (dd, J = 6.5, 2.6 Hz, 6 |
| | H) 1.22 (d, J = 6.8 Hz, 3 H) 2.37 (br d, J = 16.5 Hz, 1 H) 2.57 (dd, J = 16.6, |
| | 6.3 Hz, 1 H) 2.83-2.86 (m, 3 H) 4.03 (br d, J = 18.7 Hz, 1 H) 4.14 (dq, J = |
| | 13.6, 6.7 Hz, 1 H) 4.45-4.64 (m, 2 H) 4.81 (br d, J = 7.7 Hz, 1 H) 7.03-7.44 |
| | (m, 4 H) 7.67 (d, J = 8.2 Hz, 1 H) 8.00 (d, J = 8.3 Hz, 2 H) 8.15 (br d, |
| | J = 4.8 Hz, 1 H) |
| C680 | 1H NMR (400 MHz, DMSO-d₆, 27° C.) δ ppm 1.06 (d, J = 6.6 Hz, 6 H) |
| | 1.10 (d, J = 6.7 Hz, 3 H) 2.36 (d, J = 16.1 Hz, 1 H) 2.46 (m, J = 5.7 Hz, 1 H) |
| | 2.82 (d, J = 4.5 Hz, 3 H) 3.98 (br d, J = 18.5 Hz, 1 H) 4.20 (dq, J = 13.8, 6.8 |
| | Hz, 1 H) 4.59 (d, J = 18.5 Hz, 1 H) 4.77 (quin, J = 6.3 Hz, 1 H) 5.45 (d, J = |
| | 8.1 Hz, 1 H) 7.36 (br dd, J = 14.5, 8.1 Hz, 2 H) 7.43 (t, J = 9.2 Hz, 1 H) |
| | 7.85 (ddd, J = 9.2, 4.9, 2.8 Hz, 1 H) 7.95-8.04 (m, 3 H) 8.56 (br q, J = 4.4 |
| | Hz, 1 H) 8.92 (s, 1 H) |
| C681 | 1H NMR (400 MHz, DMSO-d₆, 100° C.) δ ppm 0.60-0.69 (m, 2 H) 0.70-0.78 (m, 2 H) |
| | 1.00-1.16 (m, 6 H) 1.18-1.28 (m, 3 H) 2.30-2.43 (m, 1 |
| | H) 2.53-2.63 (m, 1 H) 2.86-2.97 (m, 1 H) 4.05 (br d, J = 18.7 Hz, 1 H) |
| | 4.10-4.26 (m, 1 H) 4.42-4.69 (m, 2 H) 4.89-5.03 (m, 1 H) 7.04-7.45 |
| | (m, 3 H) 7.62-7.80 (m, 3 H) 7.92-8.08 (m, 2 H) 8.17-8.40 (m, 1 H) |
| C682 | 1H NMR (400 MHz, DMSO-d₆, 100° C.) δ ppm 1.11 (dd, J = 6.5, 3.1 Hz, 6 |
| | H) 1.23 (d, J = 6.8 Hz, 3 H) 2.34-2.43 (m, 1 H) 2.53-2.62 (m, 1 H) 3.53-3.61 |
| | (m, 4 H) 3.62-3.70 (m, 4 H) 4.05 (br d, J = 18.7 Hz, 1 H) 4.14 (dq, |
| | J = 13.5, 6.7 Hz, 1 H) 4.46-4.65 (m, 2 H) 4.87 (br d, J = 7.6 Hz, 1 H) 7.04-7.33 |
| | (m, 1 H) 7.36 (d, J = 8.3 Hz, 2 H) 7.56-7.62 (m, 2 H) 7.62-7.67 |
| | (m, 1 H) 7.67-7.71 (m, 1 H) 7.72 (d, J = 1.8 Hz, 1 H) |
| C683 | 1H NMR (400 MHz, DMSO-d₆, 100° C.) δ ppm 0.99-1.11 (m, 6 H) 1.20 |
| | (d, J = 6.8 Hz, 3 H) 2.30-2.39 (m, 1 H) 2.52-2.62 (m, 1 H) 2.89 (d, J = |
| | 4.9 Hz, 3 H) 4.03 (br d, J = 18.7 Hz, 1 H) 4.10-4.25 (m, 1 H) 4.41-4.64 |
| | (m, 2 H) 5.46 (br d, J = 7.5 Hz, 1 H) 7.67-7.92 (m, 4 H) 8.16 (d, J = 8.3 |
| | Hz, 1 H) 8.33-8.58 (m, 2 H) |
| C684 | 1H NMR (400 MHz, DMSO-d₆, 100° C.) δ ppm 0.97-1.13 (m, 6 H) 1.21 |
| | (d, J = 6.8 Hz, 3 H) 2.34 (br d, J = 16.5 Hz, 1 H) 2.53-2.63 (m, 1 H) 2.86-3.04 |
| | (m, 3 H) 4.06 (br d, J = 18.9 Hz, 1 H) 4.21 (dq, J = 13.6, 7.0 Hz, 1 H) |
| | 4.34-4.78 (m, 2 H) 5.86 (br d, J = 7.5 Hz, 1 H) 7.69-7.82 (m, 2 H) 7.85 |
| | (s, 1 H) 7.92-8.00 (m, 1 H) 8.36 (d, J = 8.8 Hz, 1 H) 8.91-9.06 (m, 1 H) |
| C673a | 1H NMR (400 MHz, DMSO-d₆, 100° C.) δ ppm 0.58-0.66 (m, 2 H) 0.66-0.75 (m, 2 H) |
| | 1.06 (dd, J = 6.6, 2.9 Hz, 6 H) 1.19 (d, J = 6.6 Hz, 3 H) 2.33 |
| | (d, J = 16.4 Hz, 1 H) 2.53-2.61 (m, 1 H) 2.86-2.93 (m, 1 H) 4.02 (br d, |
| | J = 18.8 Hz, 1 H) 4.19 (dq, J = 13.6, 6.8 Hz, 1 H) 4.51 (br d, J = 37.8 Hz, 2 |
| | H) 5.37 (br d, J = 7.8 Hz, 1 H) 7.48 (d, J = 8.2 Hz, 1 H) 7.74-7.77 (m, 1 H) |
| | 7.77-7.80 (m, 1 H) 7.85 (d, J = 1.8 Hz, 1 H) 7.92 (dd, J = 8.1, 2.0 Hz, 1 H) |
| | 8.06 (d, J = 1.9 Hz, 1 H) 8.38 (br d, J = 4.1 Hz, 1 H) |
| C673b | 1H NMR (400 MHz, DMSO-d₆, 100° C.) δ ppm 0.58-0.67 (m, 2 H) 0.67-0.75 (m, 2 H) |
| | 1.06 (dd, J = 6.6, 2.2 Hz, 6 H) 1.19 (d, J = 6.8 Hz, 3 H) 2.34 |
| | (d, J = 16.4 Hz, 1 H) 2.50-2.59 (m, 2 H) 2.90 (td, J = 7.4, 3.8 Hz, 1 H) |
| | 4.05 (br d, J = 18.7 Hz, 1 H) 4.18 (dq, J = 13.6, 6.8 Hz, 1 H) 4.52 (s, 2 H) |
| | 5.37 (br d, J = 7.9 Hz, 1 H) 7.44 (d, J = 8.2 Hz, 1 H) 7.71-7.76 (m, 1 H) |
| | 7.76-7.81 (m, 1 H) 7.84 (d, J = 1.8 Hz, 1 H) 7.92 (dd, J = 8.1, 2.0 Hz, 1 H) |
| | 8.06 (d, J = 1.9 Hz, 1 H) 8.38 (br d, J = 4.2 Hz, 1 H) |
| C685a | 1H NMR (400 MHz, DMSO-d₆, 101° C.) δ ppm 1.10 (dd, J = 6.6, 2.4 Hz, 6 |
| | H) 1.22 (d, J = 6.8 Hz, 3 H) 2.37 (d, J = 16.5 Hz, 1 H) 2.56-2.64 (m, 1 H) |
| | 2.87 (d, J = 4.6 Hz, 3 H) 4.04 (br d, J = 18.8 Hz, 1 H) 4.19 (d, J = 6.9 Hz, 1 |
| | H) 4.43-4.55 (m, 1 H) 4.56-4.66 (m, 1 H) 5.11-5.18 (m, 1 H) 7.17 (t, |
| | J = 54.2 Hz, 1 H) 7.50 (d, J = 8.2 Hz, 1 H) 7.62-7.66 (m, 1 H) 7.67 (s, 1 |
| | H) 7.73 (s, 1 H) 7.94 (dd, J = 8.2, 1.9 Hz, 1 H) 8.08 (d, J = 1.9 Hz, 1 H) |
| | 8.19-8.29 (m, 1 H) |
| C685b | 1H NMR (400 MHz, DMSO-d₆, 81° C.) δ ppm 1.07 (dd, J = 6.6, 2.7 Hz, 6 |
| | H) 1.20 (d, J = 6.8 Hz, 3 H) 2.35 (br d, J = 16.6 Hz, 1 H) 2.52-2.59 (m, 1 |
| | H) 2.85 (d, J = 4.5 Hz, 3 H) 4.06 (br d, J = 18.9 Hz, 1 H) 4.21 (br dd, J = |
| | 13.7, 6.8 Hz, 1 H) 4.32-4.76 (m, 2 H) 5.54 (d, J = 8.0 Hz, 1 H) 7.21 (t, J = |
| | 54.1 Hz, 1 H) 7.49 (d, J = 8.2 Hz, 1 H) 7.62-7.67 (m, 1 H) 7.70 (d, J = |
| | 10.2 Hz, 2 H) 7.94 (dd, J = 8.2, 1.9 Hz, 1 H) 8.08 (d, J = 1.9 Hz, 1 H) 8.47 |
| | (br d, J = 5.1 Hz, 1 H) |
| C686 | 1H NMR (400 MHz, DMSO-d₆, 101° C.) δ ppm 1.07 (dd, J = 6.6, 3.0 Hz, 6 |
| | H) 1.21 (d, J = 6.8 Hz, 3 H) 2.35 (d, J = 16.4 Hz, 1 H) 2.58 (br dd, J = 16.5, |
| | 5.5 Hz, 1 H) 2.85 (d, J = 4.6 Hz, 3 H) 4.05 (br d, J = 18.6 Hz, 1 H) 4.15 (dq, |
| | J = 13.5, 6.8 Hz, 1 H) 4.38-4.63 (m, 2 H) 4.94 (br d, J = 7.8 Hz, 1 H) 7.34 |
| | (d, J = 8.2 Hz, 2 H) 7.78 (dd, J = 8.4, 2.0 Hz, 1 H) 7.82 (d, J = 8.4 Hz, 1 H) |
| | 7.99 (d, J = 8.6 Hz, 2 H) 8.02 (d, J = 1.9 Hz, 1 H) 8.24 (br d, J = 5.3 Hz, 1 |
| | H) |
| C687 | 1H NMR (400 MHz, DMSO-d₆, 100° C.) δ ppm 1.05-1.11 (m, 6 H) 1.19 |
| | (d, J = 6.8 Hz, 3 H) 2.33 (br d, J = 16.5 Hz, 1 H) 2.51-2.60 (m, 1 H) 2.80 |
| | (d, J = 4.7 Hz, 3 H) 4.01 (br d, J = 18.8 Hz, 1 H) 4.12 (dq, J = 13.5, 6.7 Hz, |
| | 1 H) 4.34-4.74 (m, 2 H) 5.59 (br d, J = 7.7 Hz, 1 H) 6.76 (s, 1 H) 7.71-7.76 (m, 1 H) |
| | 7.76-7.81 (m, 1 H) 7.84 (d, J = 1.9 Hz, 1 H) 8.06-8.27 (m, |
| | 1 H) 10.71-12.50 (m, 1 H) |
| C688 | 1H NMR (400 MHz, DMSO-d₆, 101° C.) δ ppm 1.10 (dd, J = 6.6, 2.3 Hz, 6 |
| | H) 1.20 (d, J = 6.8 Hz, 3 H) 2.36 (d, J = 16.4 Hz, 1 H) 2.59 (dd, J = 16.7, |
| | 6.4 Hz, 1 H) 2.86 (d, J = 4.5 Hz, 3 H) 4.06 (br d, J = 18.8 Hz, 1 H) 4.20-4.31 |
| | (m, 1 H) 4.37-4.54 (m, 1 H) 4.54-4.70 (m, 1 H) 5.91 (br d, J = 7.9 |
| | Hz, 1 H) 7.77 (dd, J = 8.4, 1.8 Hz, 1 H) 7.80 (d, J = 8.1 Hz, 1 H) 7.87 (d, J = |
| | 1.8 Hz, 1 H) 8.05 (s, 2 H) 8.46-8.57 (m, 1 H) |
| C678a | 1H NMR (400 MHz, DMSO-d₆, 100° C.) δ ppm 0.58-0.66 (m, 2 H) 0.66-0.75 (m, 2 H) |
| | 1.04 (t, J = 6.2 Hz, 6 H) 1.19 (d, J = 6.6 Hz, 3 H) 2.32 (d, J = |
| | 16.3 Hz, 1 H) 2.50-2.59 (m, 1 H) 2.84-2.93 (m, 1 H) 3.80 (s, 3 H) 4.01 |
| | (br d, J = 18.7 Hz, 1 H) 4.14 (dq, J = 13.6, 6.8 Hz, 1 H) 4.49 (br d, J = 29.7 |
| | Hz, 2 H) 4.99 (d, J = 7.9 Hz, 1 H) 7.24 (d, J = 8.1 Hz, 1 H) 7.53 (dd, J = 8.1, |
| | 1.8 Hz, 1 H) 7.61 (d, J = 1.5 Hz, 1 H) 7.72-7.77 (m, 1 H) 7.77-7.80 (m, 1 |
| | H) 7.85 (d, J = 2.0 Hz, 1 H) 8.25 (br d, J = 4.1 Hz, 1 H) |
| C678b | 1H NMR (400 MHz, DMSO-d₆, 100° C.) δ ppm 0.58-0.67 (m, 2 H) 0.67-0.75 (m, 2 H) |
| | 1.04 (d, J = 6.6 Hz, 6 H) 1.19 (d, J = 6.8 Hz, 3 H) 2.32 (d, J = |
| | 16.3 Hz, 1 H) 2.50-2.65 (m, 1 H) 2.84-2.93 (m, 1 H) 3.81 (s, 3 H) 3.98-4.08 |
| | (m, 1 H) 4.14 (dq, J = 13.7, 6.7 Hz, 1 H) 4.49 (br s, 2 H) 5.00 (br d, |
| | J = 7.9 Hz, 1 H) 7.20 (d, J = 8.0 Hz, 1 H) 7.53 (dd, J = 8.1, 1.8 Hz, 1 H) |
| | 7.61 (d, J = 1.8 Hz, 1 H) 7.71-7.76 (m, 1 H) 7.76-7.81 (m, 1 H) 7.83 (d, |
| | J = 2.0 Hz, 1 H) 8.25 (br d, J = 4.1 Hz, 1 H) |
| C689a | 1H NMR (400 MHz, DMSO-d₆) δ ppm 1.15-1.22 (m, 3 H) 1.32-1.38 (m, |
| | 3 H) 2.31 (d, J = 16.5 Hz, 1 H) 2.52-2.60 (m, 1 H) 2.86 (d, J = 4.6 Hz, 3 |
| | H) 3.99 (br d, J = 18.0 Hz, 1 H) 4.29-4.57 (m, 2 H) 5.37 (br t, J = 7.2 Hz, 1 |
| | H) 5.78 (d, J = 7.5 Hz, 1 H) 6.94-7.06 (m, 2 H) 7.30-7.46 (m, 4 H) 7.66 |
| | (d, J = 2.0 Hz, 1 H) 7.70 (d, J = 8.1 Hz, 1 H) 8.03 (d, J = 7.9 Hz, 2 H) 8.36 |
| | (br d, J = 4.4 Hz, 1 H) |
| C689b | 1H NMR (400 MHz, DMSO-d₆) δ ppm 1.13 (d, J = 6.8 Hz, 3 H) 1.35 (d, J = |
| | 7.0 Hz, 3 H) 2.31 (d, J = 16.9 Hz, 1 H) 2.53-2.59 (m, 1 H) 2.86 (d, J = 4.6 |
| | Hz, 3 H) 3.85-3.96 (m, 1 H) 4.37-4.63 (m, 1 H) 5.41 (quin, J = 7.3 Hz, 1 |
| | H) 5.77 (d, J = 7.7 Hz, 1 H) 6.99 (t, J = 8.3 Hz, 1 H) 7.08 (ddd, J = 11.2, |
| | 9.1, 2.4 Hz, 1 H) 7.34-7.44 (m, 4 H) 7.69 (d, J = 2.0 Hz, 1 H) 7.72 (d, J = |
| | 8.1 Hz, 1 H) 8.03 (d, J = 8.6 Hz, 2 H) 8.34-8.40 (m, 1 H) |
| C690 | 1H NMR (400 MHz, DMSO-d₆, 100° C.) δ ppm 1.12 (d, J = 6.8 Hz, 3 H) |
| | 1.19 (dd, J = 6.2 Hz, 6 H) 1.54-1.68 (m, 4 H) 1.74-1.83 (m, 2 H) 2.08- |
| | 2.19 (m, 1 H) 2.25-2.32 (m, 1 H) 2.45 (br d, J = 5.5 Hz, 1 H) 2.58 (d, J = |
| | 4.6 Hz, 5 H) 3.90 (br d, J = 18.5 Hz, 1 H) 4.13-4.24 (m, 2 H) 4.42 (s, 2 H) |
| | 6.24 (br d, J = 7.2 Hz, 1 H) 7.27-7.37 (m, 1 H) 7.68-7.73 (m, 1 H) 7.74-7.79 |
| | (m, 1 H) 7.81 (d, J = 2.0 Hz, 1 H) |
| C691 | 1H NMR (400 MHz, DMSO-d₆) δ ppm 0.82 (dd, J = 36.3, 6.6 Hz, 7 H) 1.15 |
| | (d, J = 6.8 Hz, 3 H) 2.27-2.37 (m, 1 H) 2.50-2.56 (m, 1 H) 3.54-3.63 |
| | (m, 1 H) 3.66-3.78 (m, 1 H) 3.70 (s, 2 H) 3.74 (s, 3 H) 3.99 (dd, J = 59.8, |
| | 14.2 Hz, 2 H) 4.10-4.16 (m, 1 H) 4.39-4.66 (m, 1 H) 6.36 (dd, J = 8.0, |
| | 1.9 Hz, 1 H) 6.48 (d, J = 2.4 Hz, 1 H) 6.92-7.05 (m, 3 H) 7.43 (dd, J = 8.1, |
| | 2.0 Hz, 1 H) 7.61 (d, J = 8.0 Hz, 2 H) 7.70 (s, 1 H) 7.71 (d, J = 8.6 Hz, 2 H) |
| C692 | 1H NMR (400 MHz, DMSO-d₆, 100° C.) δ ppm 1.04-1.12 (m, 6 H) 1.17 |
| | (d, J = 6.8 Hz, 3 H) 2.32 (d, J = 16.3 Hz, 1 H) 2.51-2.59 (m, 1 H) 2.85 (d, |
| | J = 4.7 Hz, 3 H) 4.01 (br d, J = 18.9 Hz, 1 H) 4.17 (dq, J = 13.6, 6.8 Hz, 1 |
| | H) 4.32-4.69 (m, 2 H) 5.72 (br d, J = 7.9 Hz, 1 H) 7.41 (dd, J = 8.3, 1.9 |
| | Hz, 1 H) 7.60-7.73 (m, 3 H) 12.72-14.79 (m, 1 H) |
| C693 | 1H NMR (400 MHz, DMSO-d₆, 100° C.) δ ppm 1.01-1.10 (m, 6 H) 1.13-1.20 (m, 3 H) |
| | 1.90-2.03 (m, 3 H) 2.33 (br d, J = 16.4 Hz, 1 H) 2.51-2.60 |
| | (m, 1 H) 2.77-2.85 (m, 3 H) 4.00 (br d, J = 18.9 Hz, 1 H) 4.06-4.22 (m, 1 |
| | H) 4.24-4.80 (m, 2 H) 5.21-5.49 (m, 1 H) 7.41 (dd, J = 8.1, 1.8 Hz, 1 H) |
| | 7.52-7.75 (m, 3 H) 12.50-13.85 (m, 1 H) |
| C694 | 1H NMR (400 MHz, DMSO-d₆, 100° C.) δ ppm 1.02-1.26 (m, 15 H) 2.33 |
| | (br d, J = 16.5 Hz, 1 H) 2.52-2.61 (m, 1 H) 3.95-4.11 (m, 1 H) 4.11-4.26 (m, 2 H) |
| | 4.31-4.78 (m, 2 H) 5.95 (br d, J = 7.7 Hz, 1 H) 7.68-7.88 |
| | (m, 3 H) 8.39 (br d, J = 8.3 Hz, 1 H) 8.74 (d, J = 1.4 Hz, 1 H) 9.19 (d, J = |
| | 1.4 Hz, 1 H) |
| C695 | 1H NMR (400 MHz, DMSO-d₆, 100° C.) δ ppm 1.03-1.12 (m, 6 H) 1.20 |
| | (d, J = 6.8 Hz, 3 H) 2.34 (d, J = 16.4 Hz, 1 H) 2.56 (br dd, J = 16.3, 6.1 Hz, |
| | 1 H) 2.90 (d, J = 4.9 Hz, 3 H) 4.05 (br d, J = 18.8 Hz, 1 H) 4.20 (dq, J = |
| | 13.6, 6.8 Hz, 1 H) 4.34-4.73 (m, 2 H) 5.92 (br d, J = 7.6 Hz, 1 H) 7.70- |
| | 7.89 (m, 3 H) 8.57-8.68 (m, 1 H) 8.74 (d, J = 1.3 Hz, 1 H) 9.18 (d, J = 1.3 |
| | Hz, 1 H) |
| C696 | 1H NMR (400 MHz, DMSO-d₆, 100° C.) δ ppm 1.16 (d, J = 6.8 Hz, 3 H) |
| | 1.28 (dd, J = 10.0, 6.5 Hz, 6 H) 1.41-1.52 (m, 2 H) 1.58-1.68 (m, 2 H) |
| | 1.94 (m, J = 12.5 Hz, 2 H) 2.29-2.38 (m, 1 H) 2.52-2.65 (m, 4 H) 2.67 |
| | (d, J = 4.6 Hz, 3 H) 3.95 (br d, J = 18.5 Hz, 1 H) 4.32 (dq, J = 13.5, 6.7 Hz, |
| | 1 H) 4.46 (br s, 2 H) 5.32 (tt, J = 12.8, 6.1 Hz, 1 H) 6.69 (br d, J = 7.4 Hz, 1 |
| | H) 7.73 (dd, J = 8.3, 2.1 Hz, 2 H) 7.77-7.81 (m, 1 H) 7.83 (d, J = 2.0 Hz, 1 |
| | H) |
| C697a | 1H NMR (400 MHz, DMSO-d₆) δ ppm 1.08-1.17 (m, 3 H) 1.37-1.48 (m, |
| | 3 H) 2.26-2.36 (m, 1 H) 2.45-2.52 (m, 6 H) 2.52-2.59 (m, 1 H) 2.79-2.85 (m, 3 H) |
| | 3.74 (s, 3 H) 4.00 (br s, 1 H) 4.17-4.93 (m, 2 H) 5.28-5.41 |
| | (m, 1 H) 5.50-5.61 (m, 1 H) 6.34 (dd, J = 3.1, 0.7 Hz, 1 H) 6.91-6.98 (m, |
| | 1 H) 7.19-7.23 (m, 1 H) 7.23-7.28 (m, 1 H) 7.32-7.48 (m, 4 H) 7.67-7.69 (m, 1 H) |
| | 7.69-7.73 (m, 1 H) 7.94-8.06 (m, 2 H) 8.29-8.38 (m, 1 |
| | H) |
| C697b | 1H NMR (400 MHz, DMSO-d₆) δ ppm 1.19 (d, J = 6.8 Hz, 3 H) 1.38-1.46 |
| | (m, 3 H) 2.26-2.36 (m, 1 H) 2.52 (br s, 1 H) 2.79-2.86 (m, 3 H) 3.66-3.75 |
| | (m, 3 H) 3.95-4.15 (m, 1 H) 4.21-4.80 (m, 2 H) 5.32 (br d, J = 7.3 |
| | Hz, 1 H) 5.59 (d, J = 7.9 Hz, 1 H) 6.29-6.36 (m, 1 H) 6.89-6.97 (m, 1 H) |
| | 7.19-7.22 (m, 1 H) 7.22-7.26 (m, 1 H) 7.28-7.47 (m, 4 H) 7.64-7.66 |
| | (m, 1 H) 7.66-7.69 (m, 1 H) 7.94-8.04 (m, 2 H) 8.27-8.41 (m, 1 H) |
| C677a | 1H NMR (400 MHz, DMSO-d₆, 81° C.) δ ppm 1.23 (d, J = 6.8 Hz, 3 H) |
| | 1.35 (s, 9 H) 2.33-2.40 (m, 1 H) 2.58-2.67 (m, 1 H) 2.87 (d, J = 4.5 Hz, |
| | 3 H) 4.08 (br d, J = 20.2 Hz, 1 H) 4.31-4.81 (m, 3 H) 7.63 (d, J = 8.2 Hz, 1 |
| | H) 7.79-7.86 (m, 2 H) 7.92 (s, 1 H) 7.98 (dd, J = 8.2, 1.9 Hz, 1 H) 8.13 |
| | (d, J = 1.9 Hz, 1 H) 8.47-8.55 (m, 1 H) |
| C698 | 1H NMR (400 MHz, CHLOROFORM-d, 51° C.) δ ppm 1.00-1.15 (m, 6 H) |
| | 1.26 (d, J = 6.8 Hz, 3 H) 2.46-2.57 (m, 1 H) 2.64-2.75 (m, 1 H) 3.02 (d, |
| | J = 4.8 Hz, 3 H) 3.57-5.73 (m, 5 H) 6.37-6.71 (m, 1 H) 7.43-7.50 (m, 1 |
| | H) 7.51-7.56 (m, 1 H) 7.56-7.61 (m, 1 H) 7.78 (d, J = 1.5 Hz, 1 H) 8.23 |
| | (dd, J = 8.3, 2.4 Hz, 1 H) 8.96 (d, J = 2.4 Hz, 1 H) |
| C699 | 1H NMR (400 MHz, DMSO-d₆, 101° C.) δ ppm 0.11-0.44 (m, 4 H) 0.89 |
| | (m, J = 8.1, 8.1, 8.1, 5.0, 5.0 Hz, 1 H) 1.13 (d, J = 6.6 Hz, 3 H) 1.21 (d, J = |
| | 6.8 Hz, 3 H) 2.35 (d, J = 16.5 Hz, 1 H) 2.56 (dd, J = 16.5, 6.2 Hz, 1 H) 2.85 |
| | (d, J = 4.6 Hz, 3 H) 3.54 (sxt, J = 7.2 Hz, 1 H) 4.01 (br d, J = 18.8 Hz, 1 H) |
| | 4.42 (br d, J = 8.6 Hz, 2 H) 4.99 (d, J = 7.8 Hz, 1 H) 7.19 (t, J = 54.2 Hz, 1 |
| | H) 7.36 (d, J = 8.3 Hz, 2 H) 7.61-7.66 (m, 1 H) 7.69 (d, J = 8.4 Hz, 1 H) |
| | 7.71 (s, 1 H) 8.01 (d, J = 8.5 Hz, 2 H) 8.22-8.30 (m, 1 H) |
| C700 | 1H NMR (400 MHz, DMSO-d₆, 100° C.) δ ppm 0.57-0.64 (m, 2 H) 0.64- |
| | 0.72 (m, 2 H) 1.10 (dt, J = 6.7, 3.5 Hz, 6 H) 1.19 (dd, J = 6.8, 5.5 Hz, 3 H) |
| | 2.29-2.38 (m, 1 H) 2.56 (ddd, J = 16.6, 10.7, 6.2 Hz, 1 H) 2.79-2.88 (m, |
| | 1 H) 3.56 (d, J = 3.9 Hz, 3 H) 4.02 (br d, J = 18.9 Hz, 1 H) 4.09-4.22 (m, 1 |
| | H) 4.46 (s, 2 H) 5.93 (m, J = 6.6, 6.6 Hz, 1 H) 6.68 (d, J = 15.5 Hz, 1 H) |
| | 7.71-7.76 (m, 1 H) 7.75-7.80 (m, 2 H) 7.84 (d, J = 1.8 Hz, 1 H) |
| C701a | 1H NMR (400 MHz, DMSO-d₆, 81° C.) δ ppm 1.10-1.31 (m, 5 H) 1.76- |
| | 1.88 (m, 2 H) 2.38 (br d, J = 16.5 Hz, 1 H) 2.45-2.49 (m, 1 H) 2.57-2.72 |
| | (m, 3 H) 2.84 (d, J = 4.6 Hz, 3 H) 4.07 (br d, J = 18.9 Hz, 1 H) 4.28-4.83 |
| | (m, 3 H) 7.68 (d, J = 8.2 Hz, 1 H) 7.76-7.84 (m, 2 H) 7.89 (d, J = 1.7 Hz, 1 |
| | H) 7.92 (dd, J = 8.2, 1.9 Hz, 1 H) 8.06 (d, J = 1.9 Hz, 1 H) 8.43-8.50 (m, 1 |
| | H) |
| C701b | 1H NMR (400 MHz, DMSO-d₆, 101° C.) δ ppm 1.12-1.29 (m, 5 H) 1.77- |
| | 1.88 (m, 2 H) 2.40 (d, J = 16.5 Hz, 1 H) 2.52-2.72 (m, 4 H) 2.84 (d, J = |
| | 4.6 Hz, 3 H) 4.09 (br d, J = 18.8 Hz, 1 H) 4.40-4.70 (m, 3 H) 7.62 (d, J = |
| | 8.2 Hz, 1 H) 7.73-7.78 (m, 1 H) 7.78-7.83 (m, 1 H) 7.86 (d, J = 1.9 Hz, 1 |
| | H) 7.92 (dd, J = 8.2, 2.0 Hz, 1 H) 8.05 (d, J = 1.9 Hz, 1 H) 8.39 (br d, J = |
| | 5.3 Hz, 1 H) |
| C702 | 1H NMR (400 MHz, DMSO-d₆) δ ppm 1.19 (dd, J = 6.7, 5.0 Hz, 3 H) 2.25- |
| | 2.38 (m, 1 H) 2.50-2.61 (m, 1 H) 2.72 (d, J = 4.2 Hz, 3 H) 2.80 (d, J = |
| | 4.8 Hz, 3 H) 3.04 (s, 9 H) 3.60 (d, J = 5.3 Hz, 3 H) 4.04 (br d, J = 19.6 Hz, |
| | 1 H) 4.31-4.72 (m, 2 H) 6.45-6.54 (m, 1 H) 7.06 (d, J = 18.0 Hz, 1 H) |
| | 7.73-7.77 (m, 1 H) 7.77-7.82 (m, 1 H) 7.86 (t, J = 2.1 Hz, 1 H) 8.20- |
| | 8.29 (m, 1 H) |
| C703 | 1H NMR (400 MHz, DMSO-d₆) δ ppm 1.09 (dd, J = 6.3, 4.5 Hz, 6 H) 1.14 |
| | (br d, J = 3.3 Hz, 1 H) 1.15-1.23 (m, 9 H) 1.23-1.27 (m, 1 H) 2.27-2.38 |
| | (m, 1 H) 2.50-2.61 (m, 1 H) 3.60 (d, J = 4.6 Hz, 3 H) 3.92-4.27 (m, 2 H) |
| | 4.31-4.73 (m, 2 H) 6.12-6.23 (m, 1 H) 7.05 (d, J = 20.0 Hz, 1 H) 7.73-7.78 |
| | (m, 1 H) 7.78-7.83 (m, 1 H) 7.83-7.90 (m, 1 H) 7.96 (br d, J = 8.1 |
| | Hz, 1 H) |

### 6. Anti-HBV Activity of Compounds of Formula (I)

### Procedure

The anti HBV activity was measured using the HepG2.117 cell line, a stable, inducibly HBV producing cell line, which replicates HBV in the absence of doxycycline (Tet-off system). The HepG2 cell line is available from ATCCR under number HB-8065. Transfection of the HepG2 cell line can be as described in Sun and Nassal 2006 Journal of Hepatology 45 (2006) 636-645 “Stable HepG2- and Huh7-based human hepatoma cell lines for efficient regulated expression of infectious hepatitis B virus”.

For the antiviral assay, HBV replication was induced, followed by a treatment with serially diluted compound in 96-well plates. After 3 days of treatment, the antiviral activity was determined by quantification of intracellular HBV DNA using real-time PCR and an HBV specific primer set and probe.

Cytotoxicity of the compounds was tested using HepG2 or HepG2.117 cells, incubated for 3 or 4 days in the presence of compounds. The viability of the cells was assessed using the PERKIN ELMER ATPlite Luminescence Assay System.”

### Results

| Compound | EC₅₀ | CC₅₀ |
|---|---|---|
| number | (μM, mean value) | (μM, mean value) |
| C1 | 1.47 | >50 |
| C2 | 1.75 | >50* |
| C3 | 8.48 | >50* |
| C4 | 1.79 | 36.8* |
| C5 | 5.35 | >50* |
| C6 | 15.99 | >50* |
| C7 | 2.03 | >50* |
| C8 | 1.00 | >50 |
| C9 | 11.59 | >50* |
| C10 | 6.21 | >50* |
| C11 | 6.41 | >50* |
| C12 | 5.95 | >50* |
| C13 | 8.75 | 38.9* |
| C14 | 1.00 | >50 |
| C15 | 1.08 | >50 |
| C16 | 2.29 | 45.3* |
| C17 | 0.97 | 28.9* |
| C18 | 3.27 | >50* |
| C19 | 8.77 | >50 |
| C20 | 8.92 | 23.1* |
| C21 | 7.51 | 24.7* |
| C22 | 21.74 | 26.2* |
| C23 | 2.56 | >50* |
| C24 | 2.25 | >50* |
| C25 | 44.66 | >50 |
| C26 | 1.72 | >50 |
| C26a | 1.42 | >50 |
| C26b | 1.04 | >50 |
| C27 | 1.98 | >50 |
| C28 | 9.10 | 25.2 |
| C29 | 7.43 | 23.3 |
| C30 | >50 | >50 |
| C31 | 2.63 | >50 |
| C32 | 19.89 | 29.5 |
| C33 | 4.27 | 24.4 |
| C34 | 4.03 | 26.6 |
| C35 | 1.41 | 33.2 |
| C36 | 1.26 | >50 |
| C36Aa | 0.31 | >50 |
| C36Ab | 2.12 | >50 |
| C36Ba | 1.21 | >50 |
| C36Bb | >50 | >50 |
| C37 | 32.09 | >50 |
| C38 | 6.87 | >50 |
| C39 | 9.21 | >50 |
| C40 | 11.02 | >50 |
| C41 | 8.70 | >50 |
| C42 | 2.79 | >50 |
| C43 | 5.64 | >50 |
| C44 | 6.99 | 25.3 |
| C45 | 6.62 | 27.2 |
| C46 | 6.49 | >50 |
| C47 | 2.68 | 27.3 |
| C48 | 30.09 | >50 |
| C49 | 2.41 | 26.8 |
| C50 | 5.85 | 26.7 |
| C51 | 4.35 | 36.6 |
| C52 | 5.10 | >50 |
| C53 | 2.05 | 31.6 |
| C54 | 4.59 | 29.7 |
| C55 | 22.08 | 23.6 |
| C56 | 1.93 | >50 |
| C57 | 0.91 | >50 |
| C58 | 2.56 | 42.8 |
| C59 | 2.53 | >50 |
| C60 | 3.25 | 23.8 |
| C61 | 2.43 | 27.9 |
| C62A | 9.88 | >50 |
| C62Ba | 3.65 | >50 |
| C62Bb | 6.60 | >50 |
| C63 | 2.59 | 28.4 |
| C63A | 3.58 | 30.4 |
| C63B | 1.63 | 35.3 |
| C64 | 8.91 | >50 |
| C65 | 2.72 | 37.8 |
| C66 | 0.77 | >50 |
| C67 | 9.51 | >50 |
| C68 | 0.66 | 22.4 |
| C69 | 10.01 | 21.0 |
| C70 | 10.77 | 23.1 |
| C71 | 7.04 | >50 |
| C72 | 2.77 | 40.0 |
| C73 | 9.37 | >50 |
| C74 | 1.43 | >50 |
| C75 | >50 | >50 |
| C76 | 9.63 | 7.1 |
| C77 | 1.58 | >50 |
| C78 | 2.11 | 41.8 |
| C79 | 2.32 | 37.5 |
| C80 | 2.02 | >50 |
| C81 | 4.49 | >50 |
| C82 | 23.45 | >50 |
| C83 | 1.44 | >50 |
| C84 | 0.57 | >50 |
| C85 | 0.85 | >50 |
| C86 | 1.44 | 25.2 |
| C87 | 1.55 | >50 |
| C88 | 4.99 | >50 |
| C89 | 8.76 | >50 |
| C90 | 8.00 | 21.2 |
| C91 | 8.92 | 22.2 |
| C92 | 7.09 | 24.8 |
| C93 | 1.73 | 32.0 |
| C94 | 0.77 | >50 |
| C95 | 1.02 | >50 |
| C96 | 0.65 | 24.3 |
| C97 | 3.22 | >50 |
| C98 | 36.53 | >50 |
| C99 | 2.05 | 28.3 |
| C100 | 0.51 | >25 |
| C100A | 0.27 | >50 |
| C100B | 10.14 | >50 |
| C101a | 22.10 | 36.9 |
| C101b | 9.59 | 33.2 |
| C102 | 0.39 | >50 |
| C103 | 18.34 | >50 |
| C104 | 1.80 | 24.1 |
| C105 | 6.81 | >50 |
| C106 | 21.05 | 27.1 |
| C107 | 1.78 | 22.6 |
| C108 | 1.87 | 26.5 |
| C109 | 2.11 | >50 |
| C109A | >50 | >50 |
| C109B | ND | >50 |
| C110 | >50 | >50 |
| C110A | >50 | >50 |
| C110B | 6.38 | >50 |
| C111 | 7.64 | >50 |
| C112 | 11.46 | >50 |
| C113 | 0.65 | 19.4 |
| C114 | 1.78 | >50 |
| C115 | 37.14 | 42.8 |
| C116 | 21.59 | 27.4 |
| C117 | 9.61 | 43.5 |
| C118 | 22.01 | 39.0 |
| C119 | 6.95 | 33.5 |
| C120 | 7.36 | >50 |
| C121 | 1.55 | >50 |
| C122 | 1.44 | >50 |
| C123 | 1.23 | 24.4 |
| C124 | 36.77 | >50 |
| C125 | 1.64 | >50 |
| C126 | 1.51 | >50 |
| C127 | 1.28 | >50 |
| C128 | 1.06 | >50 |
| C129 | 0.44 | 44.4 |
| C130 | 0.48 | >50 |
| C131 | 0.53 | >50 |
| C132 | 0.70 | >50 |
| C133 | 1.94 | 28.3 |
| C134 | 4.39 | >50 |
| C135 | 1.13 | 23.3 |
| C136 | 0.24 | >50 |
| C137 | 0.30 | >50 |
| C138 | 5.98 | >50 |
| C139 | 11.22 | >50 |
| C140 | 1.88 | >50 |
| C141 | ND | >50 |
| C142 | 0.12 | >50 |
| C143 | 1.46 | >50 |
| C144 | 0.60 | >50 |
| C145 | 0.31 | >42 |
| C146 | 1.95 | >50 |
| C147 | 1.88 | >50 |
| C148 | 1.48 | >38 |
| C149 | 1.50 | 23.2 |
| C150 | 5.44 | >50 |
| C151 | 10.73 | >50 |
| C152 | 30.82 | >50 |
| C153 | 0.75 | >50 |
| C154 | 0.52 | >50 |
| C154A | 1.32 | >50 |
| C154B | 0.51 | >50 |
| C155 | 5.29 | >50 |
| C156 | 0.61 | >50 |
| C157 | 0.04 | >50 |
| C158 | 1.14 | >50 |
| C158A | 0.91 | >50 |
| C158B | 2.13 | 35.7 |
| C159 | 23.06 | >50 |
| C160 | 0.54 | >50 |
| C161 | 2.40 | >50 |
| C162 | 0.47 | >50 |
| C163 | 4.45 | >50 |
| C164 | 0.24 | >50 |
| C165 | 0.05 | >50 |
| C166 | 0.11 | >50 |
| C167 | 0.06 | >50 |
| C168 | 0.24 | >50 |
| C169 | 0.15 | 30.7 |
| C170 | 0.53 | >50 |
| C171 | 3.95 | 4.6 |
| C172 | 1.66 | 5.3 |
| C173 | 0.88 | 7.8 |
| C174 | 0.42 | 6.8 |
| C175 | 1.25 | 17.0 |
| C176 | 0.57 | 8.7 |
| C177 | 0.26 | 18.2 |
| C178 | 0.04 | >50 |
| C179 | 0.72 | 19.3 |
| C180 | 0.72 | 5.6 |
| C181 | 2.56 | >50 |
| C182 | 3.14 | >50 |
| C183 | 0.21 | >50 |
| C184 | 0.71 | >50 |
| C185 | 0.80 | 18.9 |
| C186 | 0.51 | >50 |
| C187 | 0.71 | >45 |
| C188 | 0.51 | >50 |
| C189 | 0.10 | 9.8 |
| C190 | 0.18 | >50 |
| C191 | 0.40 | >50 |
| C192 | 0.13 | 36.1 |
| C193 | 0.62 | >50 |
| C194 | 0.55 | >50 |
| C195 | 0.16 | >50 |
| C196 | 0.17 | >43 |
| C197 | 1.81 | >50 |
| C198 | 3.25 | >50 |
| C199 | 0.30 | >50 |
| C200 | 0.20 | 35.4 |
| C201 | 0.20 | 36.8 |
| C202 | 0.19 | >50 |
| C203 | 0.15 | >50 |
| C204 | 0.54 | >50 |
| C205 | 0.13 | >50 |
| C206 | 0.16 | 48.9 |
| C207 | 0.59 | >50 |
| C208 | 0.62 | 26.3 |
| C209 | 0.82 | >50 |
| C211 | 0.15 | >50 |
| C212 | 0.26 | >50 |
| C213 | 0.36 | >50 |
| C214 | 14.55 | >50 |
| C215 | 0.21 | 31.9 |
| C216 | ND | ND |
| C217 | ND | ND |
| C218 | 0.16 | >50 |
| C210 | 0.27 | >50 |
| C219 | 0.28 | >50 |
| C220 | 0.42 | >50 |
| C221 | 1.58 | >50 |
| C222 | 1.45 | >50 |
| C223 | >10 | >50 |
| C224 | 6.71 | >50 |
| C225 | 3.46 | >50 |
| C226 | 2.30 | >50 |
| C227 | 0.18 | >50 |
| C228 | 0.27 | >50 |
| C229 | 0.11 | >50 |
| C230 | 1.70 | >50 |
| C231 | 0.15 | >50 |
| C232 | 0.15 | >50 |
| C233 | 0.35 | >50 |
| C234 | 0.39 | >50 |
| C235 | 5.44 | >50 |
| C236 | >50 | >50 |
| C237 | 0.23 | >50 |
| C238 | 0.56 | >50 |
| C239 | 0.37 | >50 |
| C240 | 0.13 | >50 |
| C241 | 0.19 | 33.1 |
| C242 | 0.28 | >50 |
| C243 | 0.23 | 28.7 |
| C244 | 0.12 | 22.9 |
| C245 | 0.17 | >50 |
| C246 | 0.19 | >50 |
| C247 | 0.12 | >50 |
| C248 | 0.13 | 31.1 |
| C249 | 0.49 | >50 |
| C250 | 0.07 | 8.9 |
| C251 | 0.25 | >50 |
| C252 | 0.04 | 25.9 |
| C253 | 0.10 | 22.9 |
| C254 | 1.99 | >50 |
| C255 | 0.09 | >50 |
| C256 | 3.26 | >50 |
| C257 | 0.84 | >50 |
| C258 | 0.05 | >50 |
| C261 | 0.34 | >50 |
| C262 | 0.11 | 22.5 |
| C263 | 0.17 | >50 |
| C264 | 0.26 | >50 |
| C265 | 0.18 | >50 |
| C266 | 13.36 | >50 |
| C267 | 0.21 | >50 |
| C268 | ND | ND |
| C269 | 0.05 | >50 |
| C270 | 0.08 | >50 |
| C271 | 0.27 | 24.9 |
| C272 | 2.39 | >50 |
| C273 | 0.31 | 33.4 |
| C274 | 0.09 | >50 |
| C275 | 1.58 | >50 |
| C276 | 1.68 | >50 |
| C277 | 1.27 | 40.1 |
| C278 | 11.00 | >50 |
| C279 | 0.31 | >46 |
| C280 | 0.10 | 24.6 |
| C281 | 0.16 | >50 |
| C282 | ND | ND |
| C283 | 5.52 | >50 |
| C284 | 0.13 | >50 |
| C285 | 0.37 | 27.6 |
| C286 | 0.12 | 29.0 |
| C287 | 0.75 | >50 |
| C288 | 0.07 | >50 |
| C289 | 0.08 | >50 |
| C290 | 0.01 | 27.6 |
| C291 | 1.98 | >50 |
| C292 | 0.04 | >50 |
| C293 | 0.06 | 34.9 |
| C293b | 0.06 | 27.1 |
| C293a | 0.06 | 33.4 |
| C294 | 0.05 | 26.5 |
| C295 | 0.22 | 37.2 |
| C297 | 0.05 | >50 |
| C300a | 0.06 | >50 |
| C300b | 0.09 | >50 |
| C302 | 0.06 | >50 |
| C303 | 0.07 | 26.7 |
| C304 | 0.07 | >50 |
| C305 | 0.07 | >50 |
| C306 | 0.08 | 19.3 |
| C307 | 0.09 | 28.0 |
| C308 | 0.11 | >50 |
| C308a | 0.11 | >50 |
| C308b | 0.16 | >50 |
| C311 | 0.09 | >50 |
| C312 | 0.26 | >50 |
| C313 | 0.12 | 24.4 |
| C314a | 0.10 | >50 |
| C314b | 0.22 | >50 |
| C316 | 0.13 | 27.8 |
| C317 | 0.14 | >50 |
| C318 | 0.68 | 23.2 |
| C319 | 0.21 | 23.5 |
| C320 | 0.27 | >50 |
| C321 | 0.94 | >50 |
| C322 | 0.28 | 24.2 |
| C323 | 0.35 | >50 |
| C324 | 0.41 | >50 |
| C325 | 0.42 | 22.8 |
| C326 | 0.46 | >50 |
| C327 | 5.15 | >50 |
| C328 | 0.51 | >50 |
| C329 | 0.55 | >50 |
| C330 | 0.90 | >50 |
| C331 | 0.95 | >50 |
| C332 | 1.46 | 26.0 |
| C333 | 1.64 | >43 |
| C334 | 1.89 | >50 |
| C336 | 7.66 | >50 |
| C337 | ND | ND |
| C338 | 0.10 | >50 |
| C339 | 0.57 | >50 |
| C340a | 0.10 | 23.2 |
| C340b | 0.39 | 29.8 |
| C342 | 0.37 | 16.0 |
| C343 | 0.19 | 13.7 |
| C344 | 0.15 | >50 |
| C345 | 0.32 | 21.6 |
| C346 | 0.13 | 36.3 |
| C347 | 0.27 | 24.2 |
| C348 | 0.20 | 31.2 |
| C349 | 0.32 | 31.7 |
| C350 | 0.23 | >50 |
| C351 | 0.20 | >50 |
| C352 | 0.29 | 27.5 |
| C353 | 0.11 | 19.5 |
| C354 | 0.16 | >50 |
| C355 | 0.09 | 34.2 |
| C356 | >50 | >50 |
| C357 | ND. | >50 |
| C358 | >50 | >50 |
| C359 | >50 | >50 |
| C360 | >50 | >50 |
| C361 | >50 | >50 |
| C362 | >50 | >50 |
| C363 | >50 | >50 |
| C364 | >7.2 | >50 |
| C365 | >10 | >50 |
| C366 | >10 | >50 |
| C367 | >10 | >50 |
| C368 | ND. | >50 |
| C369 | ND. | >50 |
| C370 | 0.85 | >50 |
| C371 | 0.13 | >50 |
| C372 | 11.67 | >50 |
| C373 | 0.71 | 28.3 |
| C374 | 1.49 | >50 |
| C375a | 3.19 | >50 |
| C375b | 0.62 | >50 |
| C376 | 0.09 | >50 |
| C372b | 2.54 | >50 |
| C372a | 5.42 | >50 |
| C377b | 3.95 | >50 |
| C378 | 5.73 | >50 |
| C377a | 0.82 | >50 |
| C379 | 0.22 | 22.1 |
| C380 | 0.10 | 15.7 |
| I70 | 3.60 | >50 |
| C381 | 0.95 | 31.8 |
| C382 | 0.05 | >50 |
| C383a | 0.32 | >50 |
| C383b | 0.17 | >45 |
| C384 | 0.75 | >50 |
| C385 | 0.86 | >50 |
| C386a | 0.08 | >50 |
| C386b | 0.10 | >50 |
| C386 | 0.12 | >50 |
| C387a | 0.05 | >50 |
| C387b | 0.06 | 36.8 |
| C387 | 0.06 | 38.0 |
| C388 | 0.62 | >50 |
| C389 | >10 | >50 |
| C390 | 0.08 | 26.5 |
| C391 | 0.18 | >50 |
| C392a | 0.16 | 22.7 |
| C392b | 0.12 | 26.7 |
| C393 | 0.17 | 24.1 |
| C394a | 32.49 | >50 |
| C395 | 0.15 | >50 |
| C396 | 0.08 | 39.5 |
| C346b | 0.22 | >50 |
| C346a | 0.94 | 27.6 |
| C394b | 0.15 | 29.0 |
| C397 | 0.05 | 25.2 |
| C398 | 0.10 | 25.3 |
| C399 | 0.24 | 25.2 |
| C400 | 1.15 | >50 |
| C347a | 0.10 | 35.1 |
| C347b | 0.27 | >50 |
| C401b | 0.83 | 18.2 |
| C401a | 0.36 | 13.8 |
| C402a | 0.32 | 13.8 |
| C402b | 0.08 | 18.8 |
| C403a | 0.06 | 36.7 |
| C403b | 0.08 | 31.1 |
| C393a | 0.12 | 14.1 |
| C393b | 0.24 | 31.7 |
| C404 | 0.13 | >50 |
| C399a | 0.14 | 27.7 |
| C399b | 0.16 | 27.01 |
| C405 | >8.6 | >50 |
| C406b | 0.16 | 24.5 |
| C406a | 0.18 | 31.3 |
| C407 | 0.15 | 18.0 |
| C408 | 0.05 | >50 |
| C409 | 0.11 | >50 |
| C410 | 0.13 | 32.6 |
| C411a | 1.02 | 47.4 |
| C412 | 1.60 | >50 |
| C413 | 0.15 | 6.5 |
| C414b | 0.06 | >50 |
| C415a | 1.06 | 32.9 |
| C416b | 0.06 | 34.5 |
| C415b | 0.12 | 27.6 |
| C416a | 0.96 | >50 |
| C411b | 0.07 | 42.1 |
| C417b | 0.06 | 7.3 |
| C417a | 1.22 | 41.8 |
| C414a | 0.29 | >50 |
| C418 | 0.08 | >50 |
| C419 | 0.19 | >50 |
| C420 | 0.61 | 25.0 |
| C421 | >8.1 | >50 |
| C422b | 0.28 | 17.2 |
| C422a | 7.77 | 10.5 |
| C423 | >7.4 | >50 |
| C424 | 0.93 | >50 |
| C412a | 0.94 | >50 |
| C412b | >10 | >50 |
| C413a | 0.06 | 9.0 |
| C413b | 4.13 | 8.3 |
| C425 | 0.12 | >50 |
| C420a | 0.72 | 21.7 |
| C420b | 0.35 | 23.5 |
| C427 | 0.05 | >50 |
| C428 | 0.03 | >50 |
| C429 | 0.06 | >50 |
| C430 | 0.06 | >50 |
| C431 | 0.03 | 20.6 |
| C432 | 0.10 | 21.4 |
| C433 | 0.33 | >50 |
| C434 | 0.57 | >50 |
| C435b | 1.29 | 45.8 |
| C435a | 0.10 | 26.4 |
| C436b | 0.36 | 22.3 |
| C436a | 0.99 | 22.3 |
| C437 | 0.44 | 28.1 |
| C438 | 0.40 | 31.2 |
| C439b | >10 | >50 |
| C439a | >10 | >50 |
| C434b | 0.84 | >50 |
| C434a | 0.99 | >50 |
| C440b | 0.07 | 6.3 |
| C440a | 5.42 | 25.0 |
| C441a | 0.22 | >50 |
| C441b | 2.75 | >50 |
| C442 | 0.21 | >50 |
| C433b | 0.73 | >50 |
| C433a | 1.89 | >50 |
| C443 | 0.45 | 7.20 |
| C444 | 0.06 | 37.57 |
| C445 | 0.28 | 24.1 |
| C446 | 0.03 | 27.6 |
| C447 | 0.10 | >50 |
| C448 | 0.12 | 27.9 |
| C449a | 0.04 | >50 |
| C449b | 0.20 | 46.4 |
| C450a | 0.02 | >50 |
| C450b | 0.18 | >50 |
| C437b | 0.91 | 29.4 |
| C437a | 0.16 | 24.6 |
| C438b | 0.15 | 29.9 |
| C438a | 0.73 | 29.2 |
| C451 | 0.41 | >50 |
| C452 | 2.91 | >50 |
| C453 | 5.81 | >50 |
| C447a | 0.23 | >50 |
| C447b | 0.05 | >50 |
| C455a | 0.05 | 17.6 |
| C455b | 0.10 | >50 |
| C456 | 0.40 | >50 |
| C457 | 0.10 | 38.1 |
| C458 | 0.06 | 24.8 |
| C459 | 0.24 | 28.6 |
| C452a | 1.44 | >25 |
| C452b | >10 | >50 |
| C460 | 0.09 | >50 |
| C410b | 0.11 | >50 |
| C410a | 0.95 | >50 |
| C461 | 1.45 | >50 |
| C462a | 1.24 | >50 |
| C462b | 0.37 | >50 |
| C459a | 0.44 | 31.9 |
| C459b | 0.63 | 32.5 |
| C463a | 0.24 | >50 |
| C464 | 0.62 | |
| C465 | 0.65 | 22.6 |
| C466 | 1.43 | 31.5 |
| C467 | 0.10 | 30.0 |
| C468 | 18.84 | >50 |
| C469 | >25 | >50 |
| C470 | >25 | >50 |
| C471 | 0.25 | 21.56 |
| C472 | 0.92 | >50 |
| C473 | >25 | >50 |
| C474 | >25 | >50 |
| C475 | 0.07 | >50 |
| C476 | 0.12 | >50 |
| C477 | 10.23 | >50 |
| C478 | 1.28 | >50 |
| C479 | 3.81 | >50 |
| C480 | 10.34 | >50 |
| C481 | >25 | >50 |
| C482 | 8.34 | >50 |
| C483 | >12.5 | >25 |
| C484 | 3.09 | >50 |
| C485 | 11.93 | 24.9 |
| C486 | 17.34 | >50 |
| C487 | 0.25 | >50 |
| C488 | 7.31 | 28.2 |
| C489 | >25 | >50 |
| C490 | 0.89 | 28.4 |
| C491 | 15.33 | >50 |
| C492 | 1.98 | >50 |
| C493 | 15.17 | >50 |
| C494 | 2.64 | >50 |
| C495 | >25 | >50 |
| C496 | >25 | >50 |
| C497 | 11.83 | >50 |
| C498 | 2.92 | >50 |
| C499 | 1.53 | >50 |
| C500 | 0.59 | >50 |
| C501 | 0.36 | >50 |
| C502 | 8.95 | >50 |
| C503 | 6.32 | >50 |
| C504 | 1.01 | >50 |
| C505 | 0.02 | 25.6 |
| C506 | 0.94 | >50 |
| C507 | 2.89 | >50 |
| C508 | 0.88 | >50 |
| C509 | >25 | >50 |
| C510 | 9.42 | >50 |
| C511 | 2.41 | >50 |
| C512 | 1.11 | >50 |
| C513 | 22.14 | >50 |
| C514 | 0.61 | >50 |
| C515 | 1.14 | >50 |
| C516 | 2.68 | >50 |
| C517 | >25 | >50 |
| C518 | 0.47 | >50 |
| C519 | 0.02 | >50 |
| C520 | 3.74 | >50 |
| C521 | 7.64 | >50 |
| C522 | 0.77 | >50 |
| C523 | 18.02 | >50 |
| C524 | 1.36 | >50 |
| C525 | 0.15 | >50 |
| C526a | 0.28 | >50 |
| C526b | 5.42 | 16.3 |
| C527a | 0.39 | >50 |
| C527b | 0.65 | >50 |
| C528a | 3.38 | >50 |
| C528b | >10 | >50 |
| C529a | 0.16 | >50 |
| C529b | 0.72 | >50 |
| C463b | 7.21 | >50 |
| C530a | >7.4 | >50 |
| C530b | >10 | >50 |
| C531 | 0.21 | 26.3 |
| C532 | 0.16 | >50 |
| C533 | 0.11 | >50 |
| C534 | 0.14 | >50 |
| C535a | ND. | ND. |
| C535b | 0.39 | 36.7 |
| C536 | 0.40 | 48.1 |
| C537 | 0.08 | >50 |
| C538 | 20.60 | >50 |
| C539 | 5.47 | >50 |
| C540 | 15.82 | >50 |
| C541 | >25 | >50 |
| C542 | 12.87 | >50 |
| C543 | 7.02 | >50 |
| C544 | 0.60 | 26.7 |
| C545 | >25 | >50 |
| C546 | 2.64 | >50 |
| C547 | 0.97 | 47.2 |
| C548 | 0.58 | 10.3 |
| C549 | 1.22 | >50 |
| C550 | 0.54 | 30.2 |
| C551 | >25 | >50 |
| C552 | >25 | >50 |
| C553 | 23.74 | >50 |
| C554 | 1.77 | >50 |
| C555 | 1.33 | >50 |
| C556 | 0.57 | 25.6 |
| C557 | >25 | >50 |
| C558 | 0.46 | >50 |
| C559 | 0.60 | >50 |
| C560 | 2.59 | >50 |
| C561 | >25 | >50 |
| C562 | 2.87 | 26.8 |
| C563 | 0.26 | >50 |
| C564 | 0.02 | 21.7 |
| C565 | 0.68 | >50 |
| C566 | 0.03 | 6.8 |
| C567 | 0.67 | 36.5 |
| C568 | 0.02 | 25.8 |
| C569 | 0.01 | 21.4 |
| C570 | 0.01 | 24.5 |
| C571 | 0.09 | >50 |
| C572 | 0.01 | 14.6 |
| C573 | 0.32 | >50 |
| C574 | 0.73 | >50 |
| C575 | 0.55 | >50 |
| C576 | 0.05 | >50 |
| C577 | 0.17 | >50 |
| C578 | 0.11 | 33.2 |
| C579 | 0.12 | >50 |
| C580 | 0.08 | >50 |
| C581 | 0.15 | >50 |
| C582 | 2.34 | >50 |
| C583 | 0.21 | >50 |
| C584 | 0.10 | >50 |
| C585 | 0.10 | >50 |
| C586 | 0.21 | >50 |
| C587 | 0.09 | 9.2 |
| C588 | 1.61 | >50 |
| C589 | 0.04 | >50 |
| C590 | 12.44 | >50 |
| C591 | 0.01 | 23.2 |
| C592 | 0.70 | >50 |
| C593 | >25 | >50 |
| C594 | 0.04 | 26.1 |
| C595 | >25 | >50 |
| C596 | 0.40 | 25.5 |
| C597 | 9.49 | >50 |
| C598 | >25 | >50 |
| C599 | >25 | >50 |
| C600 | >25 | >50 |
| C601 | 7.71 | >50 |
| C602 | 0.38 | >25 |
| C603 | 0.21 | >50 |
| C604 | >25 | >50 |
| C605 | 0.15 | >50 |
| C606 | >0.5 | >10 |
| C625 | 0.18 | >50 |
| C607 | 0.14 | 11.4 |
| C608b | 1.74 | 13.0 |
| C609 | 0.08 | 18.7 |
| C610 | 0.17 | >50 |
| C608a | 0.77 | 15.6 |
| C611 | 0.17 | >50 |
| C612 | 0.14 | >50 |
| C613 | 0.20 | 19.2 |
| C614 | 0.09 | >50 |
| C615 | 0.13 | >50 |
| C616 | 0.19 | >50 |
| C617 | 0.08 | >50 |
| C618 | 0.09 | >45 |
| C619 | 0.20 | >50 |
| C620 | 0.17 | >50 |
| C621 | >2.5 | 5.99 |
| C622 | 0.07 | >50 |
| C623 | 0.04 | >50 |
| C624 | 0.06 | >50 |
| C626 | >2.5 | >50 |
| C627 | 0.12 | 33.6 |
| C628 | 0.05 | 23.2 |
| C630 | 0.11 | 20.7 |
| C631 | 0.13 | 23.9 |
| C632 | 0.12 | 6.2 |
| C633 | ND. | ND. |
| C634 | 0.63 | >50 |
| C635 | 0.07 | >50 |
| C636 | 0.23 | >35 |
| C637 | 0.16 | >50 |
| C638 | 0.14 | >50 |
| C639a | 0.02 | >50 |
| C639b | 0.02 | 25.4 |
| C640 | 0.07 | 5.2 |
| C641 | 1.99 | >50 |
| C642 | 0.04 | >50 |
| C643 | 0.05 | >50 |
| C644 | 0.23 | 7.5 |
| C645 | 0.04 | >50 |
| C646 | 1.62 | >50 |
| C643a | 0.04 | >50 |
| C647a | >2.5 | >50 |
| C647b | >2.5 | >50 |
| C648 | 0.22 | >50 |
| C643b | 0.05 | >50 |
| C649 | ND. | >50 |
| C650 | 0.05 | ND. |
| C651 | 0.05 | 8.8 |
| C652a | 0.84 | 6.2 |
| C652b | >2.5 | ND. |
| C653 | >2.5 | >50 |
| C654 | 0.03 | >50 |
| C655 | 0.08 | 9.1 |
| C656 | 0.26 | >50 |
| C657 | 0.03 | 36.7 |
| C658 | 0.07 | 32.2 |
| C659a | 0.12 | 6.8 |
| C659b | >2.5 | ND. |
| C660a | 1.30 | >50 |
| C660b | 0.32 | 27.5 |
| C661 | 0.07 | 25.0 |
| C662 | 0.16 | 30.5 |
| C661a | 0.07 | >50 |
| C661b | 0.06 | 25.4 |
| C663a | 0.24 | 13.2 |
| C663b | >2.5 | 11.3 |
| C664a | 0.13 | 6.7 |
| C664b | >2.5 | >50 |
| C665a | 0.49 | >50 |
| C665b | 0.65 | >50 |
| C666 | >2.5 | >50 |
| C667 | 1.82 | >50 |
| C668 | 0.01 | >50 |
| C669 | ND. | ND. |
| C669a | 0.02 | 29.8 |
| C669b | 0.04 | >50 |
| C670 | 0.37 | >50 |
| C671 | 0.05 | >50 |
| C672 | 0.09 | >50 |
| C673 | 0.01 | >50 |
| C674 | 2.04 | >50 |
| C675 | 1.60 | >50 |
| C676a | 0.03 | >50 |
| C676b | 0.02 | 28.7 |
| C677b | 0.02 | 24.0 |
| C678 | 0.04 | 45.3 |
| C679 | 0.21 | >50 |
| C680 | 0.50 | >50 |
| C681 | 0.04 | >50 |
| C682 | 0.12 | >50 |
| C683 | 0.06 | >50 |
| C684 | 0.06 | >50 |
| C673a | 0.03 | >25 |
| C673b | 0.03 | >50 |
| C685a | 0.03 | >50 |
| C685b | 0.03 | >50 |
| C686 | 0.20 | >50 |
| C687 | 0.02 | >50 |
| C688 | 0.04 | 29.3 |
| C678a | 0.04 | 43.0 |
| C678b | 0.04 | 44.1 |
| C689a | 0.20 | 16.7 |
| C689b | 0.46 | 27.3 |
| C690 | 0.03 | 25.3 |
| C691 | 2.45 | >50 |
| C692 | 0.07 | >50 |
| C693 | 0.11 | >50 |
| C694 | 0.05 | >50 |
| C695 | 0.04 | >50 |
| C696 | 0.05 | >50 |
| C697a | 0.34 | 37.1 |
| C697b | 0.06 | 18.4 |
| C677a | 0.02 | >50 |
| C698 | 0.02 | >50 |
| C699 | 0.04 | >50 |
| C700 | 0.05 | >50 |
| C701a | 0.04 | >50 |
| C701b | 0.03 | 25.4 |
| C702 | ND. | >50 |
| C703 | 0.09 | >50 |

N.D = not determined
CC₅₀ values: 3-days incubation unless marked with*
(*= 4-days incubation)

### Induction or Non-Induction of HBc Speckling

HepG2.117 cells were cultured in the presence of DMSO or test compound in absence of doxycycline.

After formaldehyde fixation and Triton-X-100 permeabilization, Hepatitis B virus core protein (HBc) was immunolabeled with a primary anti-HBc antibody. ALEXA 488-conjugated secondary antibody was used for fluorescent detection of the primary HBV Core signal. CELLMASK Deep Red and HOECHST 33258 were used for the detection of cytoplasm and nucleus respectively, which allowed the segmentation of cellular compartments.

An image analysis software that allows to detect different morphological phenotypes was used to determine the level of HBV core in the cytoplasm or nucleus (high content imaging assay).

1. A compound of Formula (I) 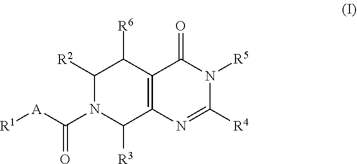
including any of its stereoisomers or tautomeric forms thereof, wherein:
A is a bond or NH;
R¹ is a 5- to 10-membered monocyclic or bicyclic ring system, more particularly a 5- to 9-membered monocyclic or bicyclic ring, wherein the 5- to 10-membered monocyclic or bicyclic ring system, more particularly the 5- to 9-membered monocyclic or bicyclic ring system, optionally contains 1 to 3 heteroatoms, the heteroatoms each independently being selected from N, O and S;
wherein the 5- to 10-membered monocyclic or bicyclic ring, more particularly the 5- to 9-membered monocyclic or bicyclic ring is optionally substituted with one or more substituents each independently selected from the group consisting of halo, CN, CF₃, CF₂H, CFH₂, CF₂CH₃, C₁₋₆alkyl, OC₁₋₆alkyl, OCF₃, OCF₂H, and C₃₋₄cycloalkyl;
or R¹ is selected from the group consisting of 1-methyl-2-oxo-1,3-dihydro-1H-benzo[d]imidazol-5-yl, 1-oxo-isoindolin-5-yl, and 1,1-dioxo-benzo[b]thiophen-5-yl;
R² is selected from the group consisting of hydrogen, C₁₋₆alkyl, CF₃, CHF₂, CH₂F, phenyl and fluorophenyl;
R³ is hydrogen;
R⁴ is X—R′;
wherein X is NR″, S or O;
wherein R′ is hydrogen, C₁₋₄alkyl, C₁₋₆alkyl substituted with OH, or C₂₋₃alkenyl, when X is NR″;
wherein R′ is C₁₋₆alkyl, when X is S;
wherein R′ is C₁₋₆alkyl, when X is O;
wherein R″ is selected from the group consisting of hydrogen, Cycle1, Aryl1, C₂₋₄alkynyl, C₁₋₆alkyl and C₁₋₆alkyl substituted with one or more substituents each independently selected from the group consisting of
fluoro,
OH,
CO₂R¹⁶,
OCONHR¹⁷,
C₃₋₆cycloalkyl,
C₃₋₆cycloalkyl substituted with one or more from among C₁₋₆alkyl,
N-acetyl piperidine,
cubanyl,
benzo[d][1,3]dioxole, and
Aryl2;
wherein R¹⁶ is hydrogen or C₁₋₆alkyl;
wherein R¹⁷ is C₁₋₆alkyl;
wherein Cycle1 is selected from the group consisting of
C₃₋₈cycloalkyl,
C₃₋₈cycloalkyl containing a heteroatom, said heteroatom being an oxygen atom,
C₃₋₈cycloalkyl substituted with one or more substituents each independently selected from CH₃ and Aryl2,
C₃₋₈cycloalkyl containing a heteroatom and being substituted with one or more substituents each independently selected from the group consisting of CH₃, cyclopropyl, and Aryl2, said heteroatom being an oxygen atom,
a 5- to 9-membered fused bicyclic unsaturated or saturated ring system, in particular a saturated heterocycle fused with an aromatic ring, which may be optionally substituted with OCH₃,
a 5- to 9-membered bridged bicyclic unsaturated or saturated ring system optionally substituted with 1, 2 or 3 CH₃ substituents,
a C₅₋₁₂spirocycloalkyl, and
cubanyl;
wherein Aryl1 is selected from the group consisting of phenyl, monocyclic heteroaryl, and bicyclic heteroaryl which is in particular an aromatic ring fused to a saturated ring or an aromatic ring fused to another aromatic ring, said Aryl1 being optionally substituted with CH₃;
wherein Aryl2 is selected from the group consisting of phenyl, monocyclic heteroaryl, and bicyclic heteroaryl, said Aryl2 being optionally substituted with one or more substituents each independently selected from the group consisting of halogens, CF₃, CF₂H, CH₂F, C₁₋₄alkyl, C₃₋₆cycloalkyl, CN, CONR¹⁸R¹⁹, OH, OCF₃, OCF₂H, OCH₂F, OC₁₋₄alkyl, OC₃₋₆cycloalkyl, SO₂CH₃, imidazolyl optionally substituted with CH₃, phenyl optionally substituted with fluoro, and triazolyl;
wherein R¹⁸ and R¹⁹ are independently selected from the group consisting of hydrogen, C₁₋₆alkyl and C₃₋₆cycloalkyl;
or wherein N, R′ and R″ together form a cycle selected from the group consisting of
a C₃₋₈cycloalkyl ring,
a C₃₋₈cycloalkyl ring containing a heteroatom, said heteroatom being an oxygen atom, and optionally being substituted with CH₃,
a C₃₋₈cycloalkyl ring substituted with one or more substituents each independently selected from the group consisting of C₁₋₆alkyl, phenyl, C₂₋₆alkynyl and C₃₋₆cycloalkyl,
a C₃₋₈cycloalkyl ring containing a heteroatom and being substituted with one or more substituents each independently selected from C₁₋₆alkyl, CN, phenyl, C₂₋₆alkynyl and C₃₋₆cycloalkyl, said heteroatom being an oxygen atom,
a C₅₋₁₂-spirocycloalkyl optionally substituted with CH₃, and
a C₅₋₆ bridged bicyclic saturated ring system;
R⁵ is selected from the group consisting of hydrogen, C₁₋₆alkyl, C₂₋₃alkenyl, Cycle2 and Aryl3;
wherein C₁₋₆alkyl is optionally substituted with one or more substituents each independently selected from the group consisting of phenyl, methoxyphenyl, OC₁₋₆alkyl, NHSO₂CH₃, C₃₋₆cycloalkyl, and C₃₋₆cycloalkyl containing a heteroatom, said heteroatom being an oxygen atom;
wherein Cycle2 is selected from the group consisting of
C₃₋₆cycloalkyl,
C₃₋₆cycloalkyl containing SO₂ or a heteroatom, the heteroatom being selected from the group consisting of oxygen and nitrogen,
C₃₋₆cycloalkyl substituted with CO₂R^{20a}, CONHR^{20b} or SO₂C₁₋₆alkyl,
C₃₋₆cycloalkyl containing SO₂ or a heteroatom and being substituted with CO₂R^{20a}, CONHR^{20b} or SO₂C₁₋₆alkyl, the heteroatom being selected from the group consisting of oxygen and nitrogen,
a 5-membered bridged bicyclic saturated ring substituted with CO₂C₁₋₆alkyl or CONHR^{20b}, cubanyl optionally substituted with CO₂C₁₋₆alkyl or CONHR^{20b},
isoindoline-1-one, and
indoline-2-one;
wherein R^{20a} is hydrogen or C₁₋₆alkyl;
wherein R^{20b} is C₁₋₆alkyl or C₃₋₆cycloalkyl;
wherein Aryl3 is selected from the group consisting of phenyl, monocyclic heteroaryl, and bicyclic heteroaryl, said Aryl3 being optionally substituted with one or more substituents each independently selected from the group consisting of halogen, C₁₋₆alkyl, CF₃, CF₂H, CH₂F, CN, OC₁₋₆alkyl, OCF₃, OCF₂H, OCH₂F, OC₃₋₆cycloalkyl, SO₂R²¹, SO₂NHR²², CO₂R²³, COR²⁴, CONR²⁵R²⁶, NHR²⁷, NHCOR²⁸, Cycle3 and Aryl4;
wherein R²¹ is C₁₋₆alkyl or C₃₋₆cycloalkyl;
wherein R²² is C₁₋₆alkyl or pyridine;
wherein R²³ is hydrogen or C₁₋₆alkyl;
wherein R²⁴ is selected from the group consisting of C₁₋₆alkyl, C₅₋₆heterocycle and C₅₋₆heterocycle substituted with CH₃;
wherein R²⁵ is hydrogen or CH₃;
wherein R²⁶ is selected from the group consisting of hydrogen,
C₁₋₆alkyl,
C₁₋₆alkyl optionally substituted with one or more substituents each independently selected from the group consisting of OH, OCH₃, NH₂, CO₂H, C₃₋₆heterocycloalkyl and C₃₋₆heterocycloalkyl substituted with CH₃,
C₃₋₆cycloalkyl;
C₃₋₆cycloalkyl containing a heteroatom, said heteroatom being an oxygen atom;
C₃₋₆cycloalkyl substituted with CO₂H; and
C₃₋₆cycloalkyl containing a heteroatom and being substituted with CO₂H, said heteroatom being an oxygen atom;
wherein R²⁷ is selected from the group consisting of
C₁₋₆alkyl,
C₁₋₆alkyl substituted with C₃₋₆heterocycloalkyl, and
C₃₋₆heterocycloalkyl;
wherein R²⁸ is C₁₋₆alkyl or C₃₋₆cycloalkyl;
wherein Cycle3 is selected from the group consisting of
C₃₋₆cycloalkyl,
C₃₋₆heterocycloalkyl,
C₃₋₆heterocycloalkyl substituted with one or more substituents each independently selected from the group consisting of OH, CH₂OH, CO₂R²⁹, NHCH₃ or NHCO₂t-Bu; and
imidazolidin-4-one substituted with CH₃;
wherein R²⁹ is hydrogen or C₁₋₆alkyl;
wherein Aryl4 is selected from the group consisting of monocyclic heteroaryl and bicyclic heteroaryl, said monocyclic or bicyclic heteroaryl being optionally substituted with one or two substituents each independently selected from the group consisting of halogens, CF₃, CH₂F, C₁₋₆alkyl, C₃₋₆cycloalkyl, OCF₃, OCH₂F, OC₁₋₆alkyl, OC₃₋₆cycloalkyl, CO₂R³⁰, SO₂CH₃, and morpholine;
wherein R³⁰ is hydrogen or C₁₋₆alkyl;
wherein R′, R″ and R⁵ are not all hydrogen; and
wherein R⁶ is hydrogen, CH₃, CF₃ or CF₂H;
or a pharmaceutically acceptable salt thereof,
for use in the prevention or treatment of an HBV infection or of an HBV-induced disease.

2. A compound of Formula (I) 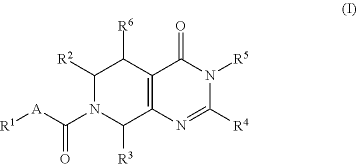
including any of its stereoisomers or tautomeric forms thereof, wherein:
A is a bond or NH;
R¹ is a 5- to 10-membered monocyclic or bicyclic ring system, more particularly a 5- to 9-membered monocyclic or bicyclic ring system, wherein the 5- to 10-membered monocyclic or bicyclic ring system, more particularly the 5- to 9-membered monocyclic or bicyclic ring system, optionally contains 1 to 3 heteroatoms, the heteroatoms each independently being selected from N, O and S;
wherein the 5- to 10-membered monocyclic or bicyclic ring, more particularly the 5- to 9-membered monocyclic or bicyclic ring is optionally substituted with one or more substituents each independently selected from the group consisting of halo, CN, CF₃, CF₂H, CFH₂, CF₂CH₃, C₁₋₆alkyl, OC₁₋₆alkyl, OCF₃, OCF₂H, and C₃₋₄cycloalkyl;
or R¹ is selected from the group consisting of 1-methyl-2-oxo-1,3-dihydro-1H-benzo[d]imidazol-5-yl, 1-oxo-isoindolin-5-yl, and 1,1-dioxo-benzo[b]thiophen-5-yl;
R² is selected from the group consisting of hydrogen, C₁₋₆alkyl, CF₃, CHF₂, CH₂F, phenyl and fluorophenyl;
R³ is hydrogen;
R⁴ is X—R′;
wherein X is NR″, S or O;
wherein R′ is hydrogen, C₁₋₄alkyl, C₁₋₆alkyl substituted with OH, or C₂₋₃alkenyl, when X is NR″;
wherein R′ is C₁₋₆alkyl, when X is S;
wherein R′ is C₁₋₆alkyl, when X is O;
wherein R″ is selected from the group consisting of hydrogen, Cycle1, Aryl1, C₂₋₄alkynyl, C₁₋₆alkyl and C₁₋₆alkyl substituted with one or more substituents each independently selected from the group consisting of
fluoro,
OH,
CO₂R¹⁶,
OCONHR¹⁷,
C₃₋₆cycloalkyl,
C₃₋₆cycloalkyl substituted with one or more from among C₁₋₆alkyl,
N-acetyl piperidine,
cubanyl,
benzo[d][1,3]dioxole, and
Aryl2;
wherein R¹⁶ is hydrogen or C₁₋₆alkyl;
wherein R¹⁷ is C₁₋₆alkyl;
wherein Cycle1 is selected from the group consisting of
C₃₋₈cycloalkyl,
C₃₋₈cycloalkyl containing a heteroatom, said heteroatom being an oxygen atom,
C₃₋₈cycloalkyl substituted with one or more substituents each independently selected from CH₃ and Aryl2,
C₃₋₈cycloalkyl containing a heteroatom and being substituted with one or more substituents each independently selected from the group consisting of CH₃, cyclopropyl, and Aryl2, said heteroatom being an oxygen atom,
a 5- to 9-membered fused bicyclic unsaturated or saturated ring, in particular a saturated heterocycle fused with an aromatic ring which may be optionally substituted with OCH₃,
a 5- to 9-membered bridged bicyclic unsaturated or saturated ring optionally substituted with 1, 2 or 3 CH₃ substituents,
a C₅₋₁₂spirocycloalkyl, and
cubanyl;
wherein Aryl1 is selected from the group consisting of phenyl, monocyclic heteroaryl, and bicyclic heteroaryl which is in particular an aromatic ring fused to a saturated ring or an aromatic ring fused to another aromatic ring, said Aryl1 being optionally substituted with CH₃;
wherein Aryl2 is selected from the group consisting of phenyl, monocyclic heteroaryl, and bicyclic heteroaryl, said Aryl2 being optionally substituted with one or more substituents each independently selected from the group consisting of halogens, CF₃, CF₂H, CH₂F, C₁₋₄alkyl, C₃₋₆cycloalkyl, CN, CONR¹⁸R¹⁹, OH, OCF₃, OCF₂H, OCH₂F, OC₁₋₄alkyl, OC₃₋₆cycloalkyl, SO₂CH₃, imidazolyl optionally substituted with CH₃, phenyl optionally substituted with fluoro, and triazolyl;
wherein R¹⁸ and R¹⁹ are independently selected from the group consisting of hydrogen, C₁₋₆alkyl and C₃₋₆cycloalkyl;
or wherein R′ and R″ together form a cycle or cycle system selected from the group consisting of
a C₃₋₈cycloalkyl ring,
a C₃₋₈cycloalkyl ring containing a heteroatom, said heteroatom being an oxygen atom, and optionally being substituted with CH₃,
a C₃₋₈cycloalkyl ring substituted with one or more substituents each independently selected from the group consisting of C₁₋₆alkyl, phenyl, C₂₋₆alkynyl and C₃₋₆cycloalkyl,
a C₃₋₈cycloalkyl ring containing a heteroatom and being substituted with one or more substituents each independently selected from the group consisting of C₁₋₆alkyl, CN, phenyl, C₂-6alkynyl and C₃₋₆cycloalkyl, said heteroatom being an oxygen atom,
a C₅₋₁₂-spirocycloalkyl optionally substituted with CH₃, and
a C₅₋₆ bridged bicyclic saturated ring system;
R⁵ is selected from the group consisting of hydrogen, C₁₋₆alkyl, C₂₋₃alkenyl, Cycle2 and Aryl3;
wherein C₁₋₆alkyl is optionally substituted with one or more substituents each independently selected from the group consisting of phenyl, methoxyphenyl, OC₁₋₆alkyl, NHSO₂CH₃, C₃₋₆cycloalkyl, and C₃₋₆cycloalkyl containing a heteroatom, said heteroatom being an oxygen atom;
wherein Cycle2 is selected from the group consisting of
C₃₋₆cycloalkyl,
C₃₋₆cycloalkyl containing SO₂ or a heteroatom, the heteroatom being selected from the group consisting of oxygen and nitrogen,
C₃₋₆cycloalkyl substituted with CO₂R^{20a}, CONHR^{20b} or SO₂C₁₋₆alkyl,
C₃₋₆cycloalkyl containing SO₂ or a heteroatom and being substituted with CO₂R^{20a}, CONHR^{20b} or SO₂C₁₋₆alkyl, the heteroatom being selected from the group consisting of oxygen and nitrogen,
a 5-membered bridged bicyclic saturated ring substituted with CO₂C₁₋₆alkyl or CONHR^{20b}, cubanyl optionally substituted with CO₂C₁₋₆alkyl or CONHR^{20b},
isoindoline-1-one, and
indoline-2-one;
wherein R^{20a} is hydrogen or C₁₋₆alkyl;
wherein R^{20b} is C₁₋₆alkyl or C₃₋₆cycloalkyl;
wherein Aryl3 is selected from the group consisting of phenyl, monocyclic heteroaryl, and bicyclic heteroaryl, said Aryl3 being optionally substituted with one or more substituents each independently selected from the group consisting of halo, C₁₋₆alkyl, CF₃, CF₂H, CH₂F, CN, OC₁₋₆alkyl, OCF₃, OCF₂H, OCH₂F, OC₃₋₆cycloalkyl, SO₂R²¹, SO₂NHR²², CO₂R²³, COR²⁴, CONR²⁵R²⁶, NHR²⁷, NHCOR²⁸, Cycle3 and Aryl4;
wherein R²¹ is C₁₋₆alkyl or C₃₋₆cycloalkyl;
wherein R²² is C₁₋₆alkyl or pyridine;
wherein R²³ is hydrogen or C₁₋₆alkyl;
wherein R²⁴ is selected from the group consisting of C₁₋₆alkyl, C₅₋₆heterocycle in particular C₅₋₆heterocycloalkyl and C₅₋₆heterocycle, in particular C₅₋₆heterocycloalkyl, substituted with CH₃;
wherein R²⁵ is hydrogen or CH₃;
wherein R²⁶ is selected from the group consisting of hydrogen,
C₁₋₆alkyl,
C₁₋₆alkyl substituted with one or more substituents each independently selected from the group consisting of OH, OCH₃, NH₂, CO₂H, C₃₋₆heterocycloalkyl and C₃₋₆heterocycloalkyl substituted with CH₃,
C₃₋₆cycloalkyl;
C₃₋₆cycloalkyl containing a heteroatom, said heteroatom being an oxygen atom;
C₃₋₆cycloalkyl substituted with CO₂H; and
C₃₋₆cycloalkyl containing a heteroatom and being substituted with CO₂H, said heteroatom being an oxygen atom;
wherein R²⁷ is selected from the group consisting of
C₁₋₆alkyl,
C₁₋₆alkyl substituted with C₃₋₆heterocycloalkyl, and
C₃₋₆heterocycloalkyl;
wherein R²⁸ is C₁₋₆alkyl or C₃₋₆cycloalkyl;
wherein Cycle3 is selected from the group consisting of
C₃₋₆cycloalkyl,
C₃₋₆heterocycloalkyl,
C₃₋₆heterocycloalkyl substituted with one or more substituents each independently selected from the group consisting of OH, CH₂OH, CO₂R²⁹, NHCH₃ or NHCO₂t-Bu; and
imidazolidin-4-one substituted with CH₃;
wherein R²⁹ is hydrogen or C₁₋₆alkyl;
wherein Aryl4 is selected from the group consisting of monocyclic heteroaryl and bicyclic heteroaryl, said monocyclic or bicyclic heteroaryl being optionally substituted with one or two substituents each independently selected from the group consisting of halo, CF₃, CH₂F, C₁₋₆alkyl, C₃-6cycloalkyl, OCF₃, OCH₂F, OC₁₋₆alkyl, OC₃₋₆cycloalkyl, CO₂R³⁰, SO₂CH₃, and morpholine;
wherein R³⁰ is hydrogen or C₁₋₆alkyl;
wherein R′, R″ and R⁵ are not all hydrogen; and R⁵ is not CH(Ph)₂ when R⁴ is NH₂; and
wherein R⁶ is hydrogen, CH₃, CF₃ or CF₂H;
or a pharmaceutically acceptable salt thereof,
with the proviso that the compound is not
2-(dimethylamino)-5,6,7,8-tetrahydro-7-[(2-methylimidazo[1,2-a]pyrimidin-3-yl)carbonyl]-Pyrido[3,4-d]pyrimidin-4(3H)-one,
7-[(2,3-dihydro-1,4-benzodioxin-6-yl)carbonyl]-2-(dimethylamino)-5,6,7,8-tetrahydro-pyrido[3,4-d]pyrimidin-4(3H)-one,
2-(dimethylamino)-5,6,7,8-tetrahydro-7-[(4,5,6,7-tetrahydrobenzo[b]thien-3-yl)-carbonyl]-pyrido[3,4-d]pyrimidin-4(3H)-one,
5,6,7,8-tetrahydro-2-(1-piperidinyl)-7-(4-thiazolylcarbonyl)-pyrido[3,4-d]pyrimidin-4(3H)-one,
7-(2-fluorobenzoyl)-5,6,7,8-tetrahydro-2-(1-piperidinyl)-pyrido[3,4-d]pyrimidin-4(3H)-one,
5,6,7,8-tetrahydro-2-(4-methyl-1-piperidinyl)-7-(4-thiazolylcarbonyl)-pyrido[3,4-d]-pyrimidin-4(3H)-one,
7-(3,4-dimethoxybenzoyl)-5,6,7,8-tetrahydro-2-(4-methyl-1-piperidinyl)-pyrido[3,4-d]pyrimidin-4(3H)-one,
5,6,7,8-tetrahydro-7-[(5-methyl-1-propyl-1H-pyrazol-4-yl)carbonyl]-2-(4-morpholinyl)-pyrido[3,4-d]pyrimidin-4(3H)-one,
7-[(1-ethyl-3-methyl-1H-pyrazol-4-yl)carbonyl]-5,6,7,8-tetrahydro-2-(4-methyl-1-piperidinyl)-pyrido[3,4-d]pyrimidin-4(3H)-one,
7-(2-furanylcarbonyl)-5,6,7,8-tetrahydro-2-(4-methyl-1-piperidinyl)-pyrido[3,4-d]-pyrimidin-4(3H)-one,
7-(3,4-dimethoxybenzoyl)-5,6,7,8-tetrahydro-2-(1-pyrrolidinyl)-pyrido[3,4-d]-pyrimidin-4(3H)-one,
5,6,7,8-tetrahydro-7-(pyrazolo[1,5-a]pyrimidin-3-ylcarbonyl)-2-(1-pyrrolidinyl)-pyrido[3,4-d]pyrimidin-4(3H)-one,
5,6,7,8-tetrahydro-7-[(5-methyl-1-propyl-1H-pyrazol-4-yl)carbonyl]-2-(1-piperidinyl)-pyrido[3,4-d]pyrimidin-4(3H)-one,
7-(3,4-dimethoxybenzoyl)-5,6,7,8-tetrahydro-2-(1-piperidinyl)-pyrido[3,4-d]pyrimidin-4(3H)-one,
5,6,7,8-tetrahydro-7-(2-pyridinylcarbonyl)-2-(1-pyrrolidinyl)-pyrido[3,4-d]pyrimidin-4(3H)-one,
2-(2,6-dimethyl-4-morpholinyl)-5,6,7,8-tetrahydro-7-(2-thienylcarbonyl)-pyrido[3,4-d]pyrimidin-4(3H)-one,
7-(cyclohexylcarbonyl)-5,6,7,8-tetrahydro-2-(1-pyrrolidinyl)-pyrido[3,4-d]pyrimidin-4(3H)-one,
5,6,7,8-tetrahydro-2-(4-methyl-1-piperidinyl)-7-[(5-methyl-1-propyl-1H-pyrazol-4-yl)-carbonyl]-pyrido[3,4-d]pyrimidin-4(3H)-one,
7-(2-furanylcarbonyl)-5,6,7,8-tetrahydro-2-(2-methyl-1-piperidinyl)-pyrido[3,4-d]-pyrimidin-4(3H)-one,
5,6,7,8-tetrahydro-2-(1-piperidinyl)-7-(2-pyrazinylcarbonyl)-pyrido[3,4-d]pyrimidin-4(3H)-one,
5,6,7,8-tetrahydro-2-(2-methyl-1-piperidinyl)-7-(2-thienylcarbonyl)-pyrido[3,4-d]-pyrimidin-4(3H)-one,
2-(2,6-dimethyl-4-morpholinyl)-7-(2-fluorobenzoyl)-5,6,7,8-tetrahydro-pyrido[3,4-d]-pyrimidin-4(3H)-one,
5,6,7,8-tetrahydro-2-(1-pyrrolidinyl)-7-(5-quinoxalinylcarbonyl)-pyrido-[3,4-d]pyrimidin-4(3H)-one,
7-[(1,2-dimethyl-1H-benzimidazol-5-yl)carbonyl]-5,6,7,8-tetrahydro-2-(4-morpholinyl)-pyrido[3,4-d]pyrimidin-4(3H)-one,
5,6,7,8-tetrahydro-2-(4-morpholinyl)-7-[(4,5,6,7-tetrahydrobenzo[b]thien-3-yl)-carbonyl]-pyrido[3,4-d]pyrimidin-4(3H)-one,
5,6,7,8-tetrahydro-2-(4-methyl-1-piperidinyl)-7-[(3-methyl-1H-pyrazol-4-yl)carbonyl]-pyrido[3,4-d]pyrimidin-4(3H)-one,
7-(2H-1-benzopyran-3-ylcarbonyl)-5,6,7,8-tetrahydro-2-(4-morpholinyl)-pyrido[3,4-d]pyrimidin-4(3H)-one,
5,6,7,8-tetrahydro-2-(4-methyl-1-piperidinyl)-7-[(4,5,6,7-tetrahydro-TH-indazol-3-yl)-carbonyl]-pyrido[3,4-d]pyrimidin-4(3H)-one,
2-(2,6-dimethyl-4-morpholinyl)-5,6,7,8-tetrahydro-7-(1H-pyrazol-3-ylcarbonyl)-pyrido[3,4-d]pyrimidin-4(3H)-one,
5,6,7,8-tetrahydro-7-(1H-pyrazol-3-ylcarbonyl)-2-(1-pyrrolidinyl)-pyrido[3,4-d]-pyrimidin-4(3H)-one,
7-(2-furanylcarbonyl)-5,6,7,8-tetrahydro-2-(1-pyrrolidinyl)-pyrido[3,4-d]pyrimidin-4(3H)-one,
7-[(1,3-dimethyl-1H-pyrazol-4-yl)carbonyl]-5,6,7,8-tetrahydro-2-(4-methyl-1-piperidinyl)-pyrido[3,4-d]pyrimidin-4(3H)-one,
7-[(1-ethyl-1H-pyrazol-4-yl)carbonyl]-5,6,7,8-tetrahydro-2-(1-pyrrolidinyl)-pyrido[3,4-d]pyrimidin-4(3H)-one,
5,6,7,8-tetrahydro-2-(4-methyl-1-piperidinyl)-7-[(4-methyl-5-thiazolyl)carbonyl]-pyrido[3,4-d]pyrimidin-4(3H)-one,
5,6,7,8-tetrahydro-7-[[5-(2-methylpropyl)-3-isoxazolyl]carbonyl]-2-(1-pyrrolidinyl)-pyrido[3,4-d]pyrimidin-4(3H)-one,
5,6,7,8-tetrahydro-7-(3-pyridinylcarbonyl)-2-(1-pyrrolidinyl)-pyrido[3,4-d]pyrimidin-4(3H)-one,
7-(2-fluorobenzoyl)-5,6,7,8-tetrahydro-2-(1-pyrrolidinyl)-pyrido[3,4-d]pyrimidin-4(3H)-one,
7-(cyclohexylcarbonyl)-2-(2,6-dimethyl-4-morpholinyl)-5,6,7,8-tetrahydro-pyrido-[3,4-d]pyrimidin-4(3H)-one,
2-(dimethylamino)-5,6,7,8-tetrahydro-7-(5-quinoxalinylcarbonyl)-pyrido[3,4-d]-pyrimidin-4(3H)-one,
2-(dimethylamino)-5,6,7,8-tetrahydro-7-[(2-propyl-4-thiazolyl)carbonyl]-pyrido[3,4-d]pyrimidin-4(3H)-one,
7-[(3-chloro-2-thienyl)carbonyl]-2-(dimethylamino)-5,6,7,8-tetrahydro-pyrido[3,4-d]pyrimidin-4(3H)-one,
2-(dimethylamino)-7-[(1-ethyl-3-methyl-3-piperidinyl)carbonyl]-5,6,7,8-tetrahydro-pyrido[3,4-d]pyrimidin-4(3H)-one,
2-(dimethylamino)-5,6,7,8-tetrahydro-7-[(tetrahydro-2-furanyl)carbonyl]-pyrido[3,4-d]pyrimidin-4(3H)-one,
2-(dimethylamino)-5,6,7,8-tetrahydro-7-(4Hthieno[3,2-b]pyrrol-5-ylcarbonyl)-pyrido[3,4-d]pyrimidin-4(3H)-one,
7-[(2,6-dimethoxy-3-pyridinyl)carbonyl]-2-(dimethylamino)-5,6,7,8-tetrahydro-pyrido[3,4-d]pyrimidin-4(3H)-one,
7-[(4-chloro-1H-pyrazol-3-yl)carbonyl]-2-(dimethylamino)-5,6,7,8-tetrahydro-pyrido[3,4-d]pyrimidin-4(3H)-one,
2-(dimethylamino)-7-[(1-ethyl-5-methyl-1Hpyrazol-4-yl)carbonyl]-5,6,7,8-tetrahydro-pyrido[3,4-d]pyrimidin-4(3H)-one,
2-(dimethylamino)-5,6,7,8-tetrahydro-7-(2-hydroxybenzoyl)-pyrido[3,4-d]pyrimidin-4(3H)-one,
2-(dimethylamino)-5,6,7,8-tetrahydro-7-[(3-methylbenzo[b]thien-2-yl)carbonyl]-pyrido[3,4-d]pyrimidin-4(3H)-one,
7-[(3-cyclohexyl-1H-pyrazol-4-yl)carbonyl]-2-(dimethylamino)-5,6,7,8-tetrahydro-pyrido[3,4-d]pyrimidin-4(3H)-one,
2-(dimethylamino)-5,6,7,8-tetrahydro-7-[(7-methylpyrazolo[1,5-a]pyrimidin-6-yl)-carbonyl]-pyrido[3,4-d]pyrimidin-4(3H)-one,
7-[(6,7-dihydro-2-methoxy-5H-cyclopenta[b]pyridin-3-yl)carbonyl]-2-(dimethyl-amino)-5,6,7,8-tetrahydro-pyrido[3,4-d]pyrimidin-4(3H)-one,
2-(dimethylamino)-7-[(2-ethyl-4-methyl-5-oxazolyl)carbonyl]-5,6,7,8-tetrahydro-pyrido[3,4-d]pyrimidin-4(3H)-one,
7-(2-chloro-3-methylbenzoyl)-2-(dimethylamino)-5,6,7,8-tetrahydro-pyrido[3,4-d]-pyrimidin-4(3H)-one,
2-(dimethylamino)-5,6,7,8-tetrahydro-7-[2-(trifluoromethyl)benzoyl]-pyrido[3,4-d]-pyrimidin-4(3H)-one,
7-[(1,2-dihydro-1,4,6-trimethyl-2-oxo-3-pyridinyl)carbonyl]-2-(dimethylamino)-5,6,7,8-tetrahydro-pyrido[3,4-d]pyrimidin-4(3H)-one,
2-(dimethylamino)-5,6,7,8-tetrahydro-7-[(6-methylimidazo[2,1-b]thiazol-5-yl)carbonyl]-pyrido[3,4-d]pyrimidin-4(3H)-one,
2-(dimethylamino)-5,6,7,8-tetrahydro-7-[(1,4,5,6-tetrahydro-3-cyclopentapyrazolyl)-carbonyl]-pyrido[3,4-d]pyrimidin-4(3H)-one,
7-[(2,3-dihydrothieno[3,4-b]-1,4-dioxin-5-yl)carbonyl]-2-(dimethylamino)-5,6,7,8-tetrahydro-pyrido[3,4-d]pyrimidin-4(3H)-one,
2-(dimethylamino)-5,6,7,8-tetrahydro-7-[[3-(2-methylpropyl)-5-isoxazolyl]carbonyl]-pyrido[3,4-d]pyrimidin-4(3H)-one,
2-(dimethylamino)-5,6,7,8-tetrahydro-7-(4-propylbenzoyl)-pyrido[3,4-d]pyrimidin-4(3H)-one,
7-[(6-chloroimidazo[1,2-a]pyridin-2-yl)carbonyl]-2-(dimethylamino)-5,6,7,8-tetrahydro-pyrido[3,4-d]pyrimidin-4(3H)-one,
2-(dimethylamino)-7-[[1-ethyl-3-(1-methylethyl)-1H-pyrazol-5-yl]carbonyl]-5,6,7,8-tetrahydro-pyrido[3,4-d]pyrimidin-4(3H)-one,
2-(dimethylamino)-5,6,7,8-tetrahydro-7-[(1-methyl-1H-indol-2-yl)carbonyl]-pyrido[3,4-d]pyrimidin-4(3H)-one,
2-(dimethylamino)-5,6,7,8-tetrahydro-7-[(2R)-2-pyrrolidinylcarbonyl]-pyrido[3,4-d]-pyrimidin-4(3H)-one,
2-(dimethylamino)-7-[(5,7-dimethyl-1,2,4-triazolo[4,3-a]pyrimidin-3-yl)carbonyl]-5,6,7,8-tetrahydro-pyrido[3,4-d]pyrimidin-4(3H)-one,
2-(dimethylamino)-5,6,7,8-tetrahydro-7-[(7-methyl-2-benzofuranyl)carbonyl]-pyrido[3,4-d]pyrimidin-4(3H)-one,
2-(dimethylamino)-5,6,7,8-tetrahydro-7-[[5-(1-methylethyl)-3-isoxazolyl]carbonyl]-pyrido[3,4-d]pyrimidin-4(3H)-one,
2-(dimethylamino)-7-[(5,7-dimethyl[1,2,4]triazolo[1,5-a]pyrimidin-2-yl)carbonyl]-5,6,7,8-tetrahydro-pyrido[3,4-d]pyrimidin-4(3H)-one,
2-(dimethylamino)-5,6,7,8-tetrahydro-7-[[4-methyl-2-(1-methylethyl)-5-pyrimidinyl]carbonyl]-pyrido[3,4-d]pyrimidin-4(3H)-one,
2-(dimethylamino)-5,6,7,8-tetrahydro-7-[(4,5,6,7-tetrahydro-5-methyl-2H-indazol-3-yl)carbonyl]-pyrido[3,4-d]pyrimidin-4(3H)-one,
7-(5-chloro-2-methoxybenzoyl)-2-(dimethylamino)-5,6,7,8-tetrahydro-pyrido[3,4-d]pyrimidin-4(3H)-one,
2-(dimethylamino)-5,6,7,8-tetrahydro-7-[(4,5,6,7-tetrahydro-5-methyl-TH-pyrazolo[4,3-c]pyridin-3-yl)carbonyl]-pyrido[3,4-d]pyrimidin-4(3H)-one,
2-(dimethylamino)-5,6,7,8-tetrahydro-7-[(1,2,3,4-tetrahydro-8-quinolinyl)carbonyl]-pyrido[3,4-d]pyrimidin-4(3H)-one,
2-(dimethylamino)-7-[[2-(ethylamino)-4-methyl-5-thiazolyl]carbonyl]-5,6,7,8-tetrahydro-pyrido[3,4-d]pyrimidin-4(3H)-one,
2-(dimethylamino)-7-[(2,7-dimethylpyrazolo[1,5-a]pyrimidin-5-yl)carbonyl]-5,6,7,8-tetrahydro-pyrido[3,4-d]pyrimidin-4(3H)-one,
5,6,7,8-tetrahydro-2-(4-methyl-1-piperidinyl)-7-(1Hpyrazol-3-ylcarbonyl)-pyrido[3,4-d]pyrimidin-4(3H)-one,
7-[[3-(1,1-dimethylethyl)-1-methyl-TH-pyrazol-5-yl]carbonyl]-5,6,7,8-tetrahydro-2-(4-morpholinyl)-pyrido[3,4-d]pyrimidin-4(3H)-one,
7-(2-fluorobenzoyl)-5,6,7,8-tetrahydro-2-(4-morpholinyl)-pyrido[3,4-d]pyrimidin-4(3H)-one,
7-[(1-ethyl-1H-pyrazol-4-yl)carbonyl]-5,6,7,8-tetrahydro-2-(4-methyl-1-piperidinyl)-pyrido[3,4-d]pyrimidin-4(3H)-one,
7-(cyclohexylcarbonyl)-5,6,7,8-tetrahydro-2-(4-morpholinyl)-pyrido[3,4-d]pyrimidin-4(3H)-one,
7-[(1-ethyl-5-methyl-TH-pyrazol-4-yl)carbonyl]-5,6,7,8-tetrahydro-2-(1-pyrrolidinyl)-pyrido[3,4-d]pyrimidin-4(3H)-one,
7-[(1,5-dimethyl-TH-pyrazol-4-yl)carbonyl]-5,6,7,8-tetrahydro-2-(1-pyrrolidinyl)-pyrido[3,4-d]pyrimidin-4(3H)-one,
5,6,7,8-tetrahydro-2-(4-methyl-1-piperidinyl)-7-(2-pyrazinylcarbonyl)-pyrido[3,4-d]-pyrimidin-4(3H)-one,
5,6,7,8-tetrahydro-2-(1-piperidinyl)-7-[(1,4,5,6-tetrahydro-3-cyclopentapyrazolyl)-carbonyl]-pyrido[3,4-d]pyrimidin-4(3H)-one,
7-[(1-ethyl-5-methyl-1H-pyrazol-4-yl)carbonyl]-5,6,7,8-tetrahydro-2-(4-methyl-1-piperidinyl)-pyrido[3,4-d]pyrimidin-4(3H)-one,
5,6,7,8-tetrahydro-2-(1-piperidinyl)-7-[(4,5,6,7-tetrahydro-1H-indazol-3-yl)carbonyl]-pyrido[3,4-d]pyrimidin-4(3H)-one,
7-[(2,3-dihydro-1,4-benzodioxin-6-yl)carbonyl]-5,6,7,8-tetrahydro-2-(1-pyrrolidinyl)-pyrido[3,4-d]pyrimidin-4(3H)-one,
7-(3,5-difluorobenzoyl)-5,6,7,8-tetrahydro-2-(1-pyrrolidinyl)-pyrido[3,4-d]pyrimidin-4(3H)-one,
5,6,7,8-tetrahydro-2-(1-pyrrolidinyl)-7-(2-thienylcarbonyl)-pyrido[3,4-d]pyrimidin-4(3H)-one,
5,6,7,8-tetrahydro-7-(1-isoquinolinylcarbonyl)-2-(4-morpholinyl)-pyrido[3,4-d]-pyrimidin-4(3H)-one,
5,6,7,8-tetrahydro-7-(1-isoquinolinylcarbonyl)-2-(1-pyrrolidinyl)-pyrido[3,4-d]-pyrimidin-4(3H)-one,
7-[(1-ethyl-1H-pyrazol-4-yl)carbonyl]-5,6,7,8-tetrahydro-2-(1-piperidinyl)-pyrido[3,4-d]pyrimidin-4(3H)-one,
5,6,7,8-tetrahydro-2-(1-piperidinyl)-7-(2-thienylcarbonyl)-pyrido[3,4-d]pyrimidin-4(3H)-one,
7-[(1,5-dimethyl-1H-pyrazol-4-yl)carbonyl]-5,6,7,8-tetrahydro-2-(4-methyl-1-piperidinyl)-pyrido[3,4-d]pyrimidin-4(3H)-one,
5,6,7,8-tetrahydro-2-(1-piperidinyl)-7-(1H-pyrazol-3-ylcarbonyl)-pyrido[3,4-d]-pyrimidin-4(3H)-one, or
7-[(2-cyclopropyl-4-quinolinyl)carbonyl]-5,6,7,8-tetrahydro-2-(4-morpholinyl)-pyrido[3,4-d]pyrimidin-4(3H)-one.

3. The compound according to claim 2, wherein
R¹ is selected from the group consisting of
phenyl optionally substituted with one or more substituents, in particular 1, 2 or 3 substituents, each independently selected from the group consisting of halo, CN, CF₃, CF₂H, CH₂F, CF₂CH₃, C₁₋₆alkyl, OC₁₋₆alkyl, OCF₃, OCF₂H, and C₃₋₄cycloalkyl;
a 5- to 6-membered heteroaryl group selected from pyridyl, thienyl, pyrrolyl and pyrazolyl, each of which is optionally substituted with one or more substituents, in particular 1 to 2 substituents, each independently selected from the group consisting of halo, CN, CF₃, C₁₋₆alkyl, OC₁₋₆alkyl, and C₃₋₄cycloalkyl, more in particular selected from the group consisting of halo, CN, CF₃, and C₁₋₆alkyl;
a 8- to 10-membered bicyclic heteroaromatic ring system selected from the group consisting of 1H-indolyl, 2,3-dihydro-1H-pyrrolo[3,2-b]pyridinyl, 1H-benzo[d]imidazolyl, benzo[b]thiophenyl,
thieno[2,3-c]pyridinyl, imidazo[1,2-a]pyridinyl, imidazo[1,5-a]pyridinyl, pyrazolo[1,5-a]pyridinyl,
1H-indazolyl, 1H-benzo[d][1,2,3]triazolyl, 1,1-dioxo-benzo[b]thiophenyl, [1,2,4]triazolo[1,5-a]pyridinyl, benzofuranyl, benzo[d]oxazolyl, benzo[d]thiazolyl, 4H-thieno[3,2-b]pyrrolyl,
isoquinolinyl,
each of which is optionally substituted with one or more substituents, in particular 1, 2 or 3 substituents, each independently selected from the group consisting of halo, CN, CF₃, C₁₋₆alkyl, OC₁₋₆alkyl, and OCF₃;
a 9- to 10-ring system selected from the group consisting of chromanyl, indolinyl, 2,3-dihydrobenzofuranyl, each optionally substituted with one or more substituents, in particular 1 or 2 substituents, each independently selected from the group consisting of halo, C₁₋₆alkyl, and OC₁₋₆alkyl;
cubanyl optionally substituted with a halo substituent;
or R¹ is selected from the group consisting of 1-methyl-2-oxo-1,3-dihydro-1H-benzo[d]imidazol-5-yl, 1-oxo-isoindolin-5-yl, and 1,1-dioxo-benzo[b]thiophen-5-yl.

4. The compound according to claim 2, wherein
R⁴ is selected from the group consisting of —OC₁₋₆alkyl, —SC₁₋₆alkyl and NR′R″, wherein
R′ is hydrogen, C₁₋₄alkyl, C₁₋₆alkyl substituted with OH, or C₂₋₃alkenyl; and
R″ is selected from the group consisting of hydrogen, Cycle1, Aryl1, C₂₋₄alkynyl, C₁₋₆alkyl and C₁₋₆alkyl substituted with one or more substituents each independently selected from the group consisting of
fluoro,
OH,
CO₂R¹⁶,
OCONHR¹⁷,
C₃₋₆cycloalkyl,
C₃₋₆cycloalkyl substituted with one or more from among C₁₋₆alkyl,
N-acetyl piperidine,
cubanyl,
benzo[d][1,3]dioxole, and
Aryl2;
wherein R¹⁶ is hydrogen or C₁₋₆alkyl;
wherein R¹⁷ is C₁₋₆alkyl;
wherein Cycle1 is selected from the group consisting of
C₃₋₈cycloalkyl
C₃₋₈cycloalkyl containing a heteroatom, said heteroatom being an oxygen atom,
C₃₋₈cycloalkyl substituted with one or more substituents each independently selected from CH₃ and Aryl2,
C₃₋₈cycloalkyl containing a heteroatom and being substituted with one or more substituents each independently selected from the group consisting of CH₃, cyclopropyl, and phenyl, said heteroatom being an oxygen atom,
a 5- to 9-membered fused bicyclic unsaturated or saturated ring, in particular a saturated heterocycle fused with an aromatic ring which may be optionally substituted with OCH₃,
a 5- to 9-membered bridged bicyclic unsaturated or saturated ring, optionally substituted with 1, 2 or 3 CH₃ substituents,
a C₇₋₉spirocycloalkyl, and
cubanyl;
wherein Aryl1 is selected from the group consisting of phenyl, monocyclic heteroaryl, and bicyclic heteroaryl which is in particular an aromatic ring fused to a saturated ring or an aromatic ring fused to another aromatic ring, said Aryl1 being optionally substituted with CH₃;
wherein Aryl2 is selected from the group consisting of
phenyl optionally substituted with one or more substituents each independently selected from the group consisting of halo, CF₃, CF₂H, CH₂F, C₁₋₄alkyl, C₃₋₆cycloalkyl, CN, CONR¹⁸R¹⁹, OH, OCF₃, OCF₂H, OCH₂F, OC₁₋₄alkyl, OC₃₋₆cycloalkyl, SO₂CH₃, imidazolyl optionally substituted with CH₃, and triazolyl;
wherein R¹⁸ and R¹⁹ are independently selected from the group consisting of hydrogen, C₁₋₆alkyl and C₃₋₆cycloalkyl;
monocyclic 5- to 6-membered heteroaryl containing 1, 2 or 3 heteroatoms each independently selected from N, O and S, and being optionally substituted with one or more substituents each independently selected from the group consisting of halo, CF₃, CF₂H, CH₂F, C₁₋₄alkyl, C₃-6cycloalkyl, OH, OCF₃, OCF₂H, OCH₂F, OC₁₋₄alkyl, OC₃₋₆cycloalkyl, and phenyl optionally substituted with fluoro;
9- to 10-membered bicyclic heteroaryl which is in particular an aromatic ring fused to a saturated ring or an aromatic ring fused to another aromatic ring, containing 1, 2 or 3 heteroatoms each independently selected from N, S, and O, and being optionally substituted with one or more substituents each independently selected from the group consisting of halo, C₁₋₄alkyl, OC₁₋₄alkyl, and C₃₋₆cycloalkyl;
or wherein NR′ and R″ together form a saturated cycle or cycle system selected from the group consisting of
a 4- to 7-membered heterocycloalkyl ring, optionally containing a further heteroatom, said heteroatom being an oxygen, and said ring being optionally substituted with CH₃,
a 4- to 7-membered heterocycloalkyl ring optionally substituted with one or more substituents each independently selected from the group consisting of C₁₋₆alkyl, phenyl, C₂₋₆alkynyl and C₃₋₆cycloalkyl,
a C₅₋₁₂-spirocycloalkyl, in particular a C₆₋₈spirocycloalkyl, optionally substituted with CH₃, and
a C₅₋₆ bridged bicyclic saturated ring system, in particular 2-azabicyclo[2.1.1]hexyl.

5. The compound according to claim 2, wherein
R⁵ is selected from the group consisting of hydrogen, C₁₋₆alkyl, Cycle2 and Aryl3;
wherein C₁₋₆alkyl is optionally substituted with one or more substituents each independently selected from the group consisting of phenyl, methoxyphenyl, OC₁₋₆alkyl, NHSO₂CH₃, and C₃₋₆cycloalkyl;
wherein Cycle2 is selected from the group consisting of
C₃₋₆cycloalkyl,
C₃₋₆cycloalkyl containing SO₂ or a heteroatom, the heteroatom being selected from the group consisting of oxygen and nitrogen,
C₃₋₆cycloalkyl substituted with CONHR^{20b} or SO₂C₁₋₆alkyl,
C₃₋₆cycloalkyl containing SO₂ or a heteroatom and being substituted with CONHR^{20b} or SO₂C₁₋₆alkyl, the heteroatom being selected from the group consisting of oxygen and nitrogen,
a 5-membered bridged bicyclic saturated ring, in particular bicyclo[1.1.1]pentanyl or bicyclo[2.1.0]pentanyl, substituted with CONHR^{20b}, and
cubanyl optionally substituted with CONHR^{20b};
wherein R^{20b} is C₁₋₆alkyl or C₃₋₆cycloalkyl;
wherein Aryl3 is selected from the group consisting of
phenyl optionally substituted with one or more substituents each independently selected from the group consisting of halo, C₁₋₆alkyl, CF₃, CF₂H, CH₂F, CN, OC₁₋₆alkyl, OCF₃, OCF₂H, OCH₂F, OC₃₋₆cycloalkyl, SO₂R²¹, SO₂NHR²², COR²⁴, CONR²⁵R²⁶, NHR²⁷, NHCOR²⁸, Cycle3 and Aryl4;
5- to 6-membered monocyclic heteroaryl selected from the group consisting of pyrazolyl, imidazolyl, oxazolyl, isoxazolyl, thiazolyl, triazolyl, pyridyl, pyridazinyl, pyrimidinyl, and pyrazinyl, each of which may be optionally substituted with one or more substituents each independently selected from the group consisting of halo, C₁₋₆alkyl, OC₁₋₆alkyl, SO₂R²¹, CONR²⁵R²⁶ and NHR²⁷; and
bicyclic heteroaryl selected from the group consisting of 1H-indolyl, 1H-indazolyl,
benzo[d]oxazolyl, and benzo[d]isoxazolyl, each of which may be optionally substituted with one or more substituents each independently selected from the group consisting of C₁₋₆alkyl, CONR²⁵R²⁶, and NHR²⁷;
wherein R²¹ is C₁₋₆alkyl or C₃₋₆cycloalkyl;
wherein R²² is C₁₋₆alkyl or pyridine;
wherein R²⁴ is selected from the group consisting of C₁₋₆alkyl, and morpholinyl or piperazinyl each of which may be optionally substituted with CH₃;
wherein R²⁵ is hydrogen or CH₃;
wherein R²⁶ is selected from the group consisting of
hydrogen,
C₁₋₆alkyl,
C₁₋₆alkyl substituted with one or more substituents each independently selected from the group consisting of OH, OCH₃, NH₂, CO₂H, and morpholinyl or piperazinyl each of which may be optionally substituted with CH₃,
C₃₋₄cycloalkyl; and
C₃₋₄cycloalkyl containing a heteroatom, said heteroatom being an oxygen atom;
wherein R²⁷ is C₁₋₆alkyl; and
wherein R²⁸ is C₁₋₆alkyl or C₃₋₆cycloalkyl;
wherein Cycle3 is selected from the group consisting of cyclopropyl,
C₃₋₆heterocycloalkyl, in particular pyrrolidinyl or morpholinyl, substituted with one or more substituents each independently selected from the group consisting of OH, CH₂OH, CO₂R²⁹, NHCH₃ or NHCO₂t-Bu; and
imidazolidin-4-one substituted with CH₃;
wherein R²⁹ is hydrogen or C₁₋₆alkyl; and
wherein Aryl4 is a monocyclic heteroaryl selected from the group consisting of furanyl, pyrazolyl, imidazolyl, oxazolyl, isoxazolyl, thiazolyl, isothiazolyl, triazolyl, tetrazolyl, oxadiazolyl, thiadiazolyl, pyridyl, pyridazinyl, pyrimidinyl, and pyrazinyl, each of which may be optionally substituted with one or two substituents each independently selected from the group consisting of halo, CF₃, CH₂F, C₁₋₆alkyl, C₃₋₆cycloalkyl, CO₂R³⁰, SO₂CH₃, and morpholine;
wherein R³⁰ is hydrogen or C₁₋₆alkyl.

6. The compound according to claim 2 wherein
A is a bond or NH;
R¹ is a 5- to 10-membered monocyclic or bicyclic ring, more particularly a 5- to 9-membered monocyclic or bicyclic ring, wherein the 5- to 10-membered monocyclic or bicyclic ring, more particularly the 5- to 9-membered monocyclic or bicyclic ring, optionally contains 1 to 3 heteroatoms, the heteroatoms independently being selected from N, O and S;
wherein the 5- to 10-membered monocyclic or bicyclic ring, more particularly the 5- to 9-membered monocyclic or bicyclic ring is optionally substituted with one or more substituents selected from halogens, CN, CF₃, CF₂H, CFH₂, CF₂CH₃, C₁₋₆alkyl, OC₁₋₆alkyl, OCF₃, OCF₂H and C₃₋₄cycloalkyl;
R² is selected from the group consisting of hydrogen, C₁₋₆alkyl, CF₃, CHF₂, CH₂F, phenyl and fluorophenyl;
R³ is hydrogen;
R⁴ is X—R′;
wherein X is NR″, S or O;
wherein R′ is hydrogen, C₁₋₄alkyl, C₁₋₆alkyl substituted with OH, or C₂₋₃alkenyl, when X is NR″;
wherein R′ is C₁₋₆alkyl, when X is S;
wherein R′ is C₁₋₆alkyl, when X is O;
wherein R″ is selected from the group consisting of hydrogen, Cycle1, Aryl1, C₂₋₄alkynyl, C₁₋₆alkyl and C₁₋₆alkyl substituted with one or more substituents selected from the group consisting of
fluoro,
OH,
CO₂R¹⁶,
OCONHR¹⁷,
C₃₋₆cycloalkyl, and C₃₋₆cycloalkyl substituted with one or more from among C₁₋₆alkyl,
N-acetyl piperidine,
benzo[d][1,3]dioxole and
Aryl2;
wherein R¹⁶ is hydrogen or C₁₋₆alkyl;
wherein R¹⁷ is C₁₋₆alkyl;
wherein Cycle1 is selected from the group consisting of
C₃₋₈cycloalkyl
C₃₋₈cycloalkyl containing a heteroatom, said heteroatom being an oxygen atom,
C₃₋₈cycloalkyl substituted with one or more substituents selected from CH₃ and Aryl2,
C₃₋₈cycloalkyl containing a heteroatom and being substituted with one or more substituents selected from CH₃ and Aryl2, said heteroatom being an oxygen atom,
a 5-9 membered fused bicyclic unsaturated or saturated ring,
a 5-9 membered bridged bicyclic unsaturated or saturated ring, and
a C₅₋₁₂spirocycloalkyl;
wherein Aryl1 is selected from the group consisting of phenyl, monocyclic heteroaryl, and bicyclic heteroaryl, said Aryl1 being optionally substituted with CH₃;
wherein Aryl2 is selected from the group consisting of phenyl, monocyclic heteroaryl, and bicyclic heteroaryl, said Aryl2 being optionally substituted with one or more substituents selected from the group consisting of halogens, CF₃, CF₂H, CH₂F, C₁₋₄alkyl, C₃₋₆cycloalkyl, CN, CONR¹⁸R¹⁹, OCF₃, OCF₂H, OCH₂F, OC₁₋₄alkyl, OC₃₋₆cycloalkyl, and SO₂CH₃;
wherein R¹⁸ and R¹⁹ are independently selected from the group consisting of hydrogen, C₁₋₆alkyl and C₃₋₆cycloalkyl;
or wherein R′ and R″ together form a cycle selected from the group consisting of
a C₃₋₈cycloalkyl ring,
a C₃₋₈cycloalkyl ring containing a heteroatom, said heteroatom being an oxygen atom,
a C₃₋₈cycloalkyl ring substituted with one or more substituents selected from C₁₋₆alkyl, phenyl, C₂₋₆alkynyl and C₃₋₆cycloalkyl,
a C₃₋₈cycloalkyl ring containing a heteroatom and being substituted with one or more substituents selected from C₁₋₆alkyl, phenyl, C₂₋₆alkynyl and C₃₋₆cycloalkyl, said heteroatom being an oxygen atom and
a C₅₋₁₂-spirocycloalkyl;
R⁵ is selected from the group consisting of hydrogen, C₁₋₆alkyl, C₂₋₃alkenyl, Cycle2 and Aryl3;
wherein C₁₋₆alkyl is optionally substituted with one or more substituents selected from the group consisting of phenyl, methoxyphenyl, OC₁₋₆alkyl, NHSO₂CH₃, C₃₋₆cycloalkyl, and C₃₋₆cycloalkyl containing a heteroatom, said heteroatom being an oxygen atom;
wherein Cycle2 is selected from the group consisting of
C₃₋₆cycloalkyl,
C₃₋₆cycloalkyl containing SO₂ or a heteroatom, the heteroatom being selected from the group consisting of oxygen and nitrogen,
C₃₋₆cycloalkyl substituted with CO₂R^{20a}, CONHC₁₋₆alkyl or SO₂C₁₋₆alkyl,
C₃₋₆cycloalkyl containing SO₂ or a heteroatom and being substituted with CO₂R^{20a}, CONHC₁₋₆alkyl or SO₂C₁₋₆alkyl, the heteroatom being selected from the group consisting of oxygen and nitrogen,
a 5-membered bridged bicyclic saturated ring substituted with CO₂C₁₋₆alkyl or CONHC₁₋₆Alkyl,
isoindoline-1-one, and
indoline-2-one;
wherein R^{20a} is hydrogen or C₁₋₆alkyl;
wherein Aryl3 is selected from the group consisting of phenyl, monocyclic heteroaryl, and bicyclic heteroaryl, said Aryl3 being optionally substituted with one or more substituents selected from the group consisting of halogen, C₁₋₆alkyl, CF₃, CF₂H, CH₂F, CN, OC₁₋₆alkyl, OCF₃, OCF₂H, OCH₂F, OC₃₋₆cycloalkyl, SO₂R²¹, SO₂NHR²², CO₂R²³, COR²⁴, CONR²¹R²⁶, NHR²⁷, NHCOR²⁸, Cycle3 and Aryl4;
wherein R²¹ is C₁₋₆alkyl or C₃₋₆cycloalkyl;
wherein R²² is C₁₋₆alkyl or pyridine;
wherein R²³ is hydrogen or C₁₋₆alkyl;
wherein R²⁴ is selected from the group consisting of C₁₋₆alkyl, C₅₋₆heterocycle and C₅₋₆heterocycle substituted with CH₃;
wherein R²⁵ is hydrogen or CH₃;
wherein R²⁶ is selected from the group consisting of hydrogen,
C₁₋₆alkyl,
C₁₋₆alkyl optionally substituted with one or more substituents selected from the group consisting of OH, OCH₃, NH₂, CO₂H, C₃₋₆heterocycloalkyl and C₃₋₆heterocycloalkyl substituted with CH₃,
C₃₋₄cycloalkyl;
C₃₋₄cycloalkyl containing a heteroatom, said heteroatom being an oxygen atom;
C₃₋₄cycloalkyl substituted with CO₂H; and
C₃₋₄cycloalkyl containing a heteroatom and being substituted with CO₂H, said heteroatom being an oxygen atom;
wherein R²⁷ is selected from the group consisting of
C₁₋₆alkyl,
C₁₋₆alkyl substituted with C₃₋₆heterocycloalkyl, and
C₃₋₆heterocycloalkyl;
wherein R²⁸ is C₁₋₆alkyl or C₃₋₆cycloalkyl;
wherein Cycle3 is selected from the group consisting of
C₃₋₆heterocycloalkyl,
C₃₋₆heterocycloalkyl substituted with one or more substituents selected from the group consisting of OH, CH₂OH, CO₂R²⁹, NHCH₃ or NHCO₂t-Bu; and
imidazolidin-4-one substituted with CH₃;
wherein R²⁹ is hydrogen or C₁₋₆alkyl;
wherein Aryl4 is selected from the group consisting of monocyclic heteroaryl and bicyclic heteroaryl, said monocyclic or bicyclic heteroaryl being optionally substituted with one or two substituents selected from the group consisting of halogens, CF₃, CH₂F, C₁₋₆alkyl, C₃₋₆cycloalkyl, OCF₃, OCH₂F, OC₁₋₆alkyl, OC₃₋₆cycloalkyl, CO₂R³⁰, SO₂CH₃, and morpholine;
wherein R³⁰ is hydrogen or C₁₋₆alkyl;
wherein R⁶ is hydrogen, CH₃, CF₃ or CF₂H.

7. The compound according to claim 2, wherein R⁵ is phenyl or phenyl substituted with one or more substituents selected from the group consisting of halogen, C₁₋₆alkyl, CF₃, CF₂H, CH₂F, CN, OC₁₋₆alkyl, OCF₃, OCF₂H, OCH₂F, OC₃₋₆cycloalkyl, SO₂R²¹, SO₂NHR²², CO₂R²³, COR²⁴, CONR²⁵R²⁶, NHR²⁷, NHCOR²⁸, Cycle3 and Aryl4.

8. The compound of claim 2, wherein A is a bond.

9. The compound of claim 2, wherein R³ and R⁶ are both hydrogen.

10. A pharmaceutical composition, which comprises the compound or pharmaceutically acceptable salt of claim 2, and which further comprises at least one pharmaceutically acceptable carrier.

11. (canceled)

12. A process for the preparation of the pharmaceutical composition according to claim 10, characterized in that at least one pharmaceutically acceptable carrier is intimately mixed with a therapeutically effective amount of a compound of Formula (I) as defined in claim 2.

13. A product comprising a first compound and a second compound as a combined preparation for simultaneous, separate or sequential use in the prevention or treatment of an HBV infection or of an HBV-induced disease in mammal in need thereof, wherein said first compound is different from said second compound, wherein said first compound is the compound or pharmaceutically acceptable salt of claim 2, and wherein said second compound is another HBV inhibitor which is selected from the group consisting of therapeutic agents selected from HBV combination drugs, HBV vaccines, HBV DNA polymerase inhibitors, immunomodulators, toll-like receptor (TLR) modulators, interferon alpha receptor ligands, hyaluronidase inhibitors, hepatitis b surface antigen (HBsAg) inhibitors, cytotoxic T-lymphocyte-associated protein 4 (ipi4) inhibitors, cyclophilin inhibitors, HBV viral entry inhibitors, antisense oligonucleotide targeting viral mRNA, short interfering RNAs (siRNA) and ddRNAi endonuclease modulators, ribonucleotide reductase inhibitors, HBV E antigen inhibitors, covalently closed circular DNA (cccDNA) inhibitors, famesoid X receptor agonists, HBV antibodies, CCR2 chemokine antagonists, thymosin agonists, cytokines, nucleoprotein modulators, retinoic acid-inducible gene 1 simulators, NOD2 stimulators, phosphatidylinositol 3-kinase (PI3K) inhibitors, indoleamine-2, 3-dioxygenase (IDO) pathway inhibitors, PD-1 inhibitors, PD-L1 inhibitors, recombinant thymosin alpha-1, bruton's tyrosine kinase (BTK) inhibitors, KDM inhibitors, HBV replication inhibitors, arginase inhibitors, and other HBV drugs.

14. The product of claim 13, which is for simultaneous, separate or sequential use in the prevention or treatment of chronic Hepatitis B.

15. A method of treating or preventing HBV infection or an HBV-induced disease in a subject in need thereof, said method comprising administering to said subject a therapeutically effective amount of a compound of Formula (I) 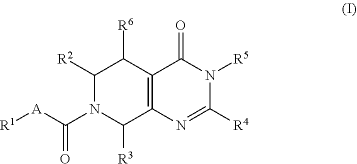
including any of its stereoisomers or tautomeric forms thereof, wherein:
A is a bond or NH;
R¹ is a 5- to 10-membered monocyclic or bicyclic ring system, more particularly a 5- to 9-membered monocyclic or bicyclic ring system, wherein the 5- to 10-membered monocyclic or bicyclic ring system, more particularly the 5- to 9-membered monocyclic or bicyclic ring system, optionally contains 1 to 3 heteroatoms, the heteroatoms each independently being selected from N, O and S;
wherein the 5- to 10-membered monocyclic or bicyclic ring system, more particularly the 5- to 9-membered monocyclic or bicyclic ring system is optionally substituted with one or more substituents each independently selected from the group consisting of halo, CN, CF₃, CF₂H, CFH₂, CF₂CH₃, C₁₋₆alkyl, OC₁₋₆alkyl, OCF₃, OCF₂H, and C₃₋₄cycloalkyl;
or R¹ is selected from the group consisting of 1-methyl-2-oxo-1,3-dihydro-1H-benzo[d]imidazol-5-yl, 1-oxo-isoindolin-5-yl, and 1,1-dioxo-benzo[b]thiophen-5-yl;
R² is selected from the group consisting of hydrogen, C₁₋₆alkyl, CF₃, CHF₂, CH₂F, phenyl and fluorophenyl;
R³ is hydrogen;
R⁴ is X—R′;
wherein X is NR″, S or O;
wherein R′ is hydrogen, C₁₋₄alkyl, C₁₋₆alkyl substituted with OH, or C₂₋₃alkenyl, when X is NR″;
wherein R′ is C₁₋₆alkyl, when X is S;
wherein R′ is C₁₋₆alkyl, when X is O;
wherein R″ is selected from the group consisting of hydrogen, Cycle1, Aryl1, C₂₋₄alkynyl, C₁₋₆alkyl and C₁₋₆alkyl substituted with one or more substituents selected from the group consisting of
fluoro,
OH,
CO₂R¹⁶,
OCONHR¹⁷,
C₃₋₆cycloalkyl,
C₃₋₆cycloalkyl substituted with one or more from among C₁₋₆alkyl,
N-acetyl piperidine,
cubanyl,
benzo[d][1,3]dioxole, and
Aryl2;
wherein R¹⁶ is hydrogen or C₁₋₆alkyl;
wherein R¹⁷ is C₁₋₆alkyl;
wherein Cycle1 is selected from the group consisting of
C₃₋₈cycloalkyl,
C₃₋₈cycloalkyl containing a heteroatom, said heteroatom being an oxygen atom,
C₃₋₈cycloalkyl substituted with one or more substituents each independently selected from CH₃ and Aryl2,
C₃₋₈cycloalkyl containing a heteroatom and being substituted with one or more substituents each independently selected from the group consisting of CH₃, cyclopropyl, and Aryl2, said heteroatom being an oxygen atom,
a 5- to 9-membered fused bicyclic unsaturated or saturated ring system, in particular a saturated heterocycle fused with an aromatic ring which may be optionally substituted with OCH₃,
a 5- to 9-membered bridged bicyclic unsaturated or saturated ring system optionally substituted with 1, 2 or 3 CH₃ substituents,
a C₅₋₁₂spirocycloalkyl, and
cubanyl;
wherein Aryl1 is selected from the group consisting of phenyl, monocyclic heteroaryl, and bicyclic heteroaryl which is in particular an aromatic ring fused to a saturated ring or an aromatic ring fused to another aromatic ring, said Aryl1 being optionally substituted with CH₃;
wherein Aryl2 is selected from the group consisting of phenyl, monocyclic heteroaryl, and bicyclic heteroaryl, said Aryl2 being optionally substituted with one or more substituents each independently selected from the group consisting of halogens, CF₃, CF₂H, CH₂F, C₁₋₄alkyl, C₃₋₆cycloalkyl, CN, CONR¹⁸R¹⁹, OH, OCF₃, OCF₂H, OCH₂F, OC₁₋₄alkyl, OC₃₋₆cycloalkyl, SO₂CH₃, imidazolyl optionally substituted with CH₃, phenyl optionally substituted with fluoro, and triazolyl;
wherein R¹⁸ and R¹⁹ are independently selected from the group consisting of hydrogen, C₁₋₆alkyl and C₃₋₆cycloalkyl;
or wherein N, R′ and R″ together form a cycle selected from the group consisting of
a C₃₋₈cycloalkyl ring,
a C₃₋₈cycloalkyl ring containing a heteroatom, said heteroatom being an oxygen atom, and optionally being substituted with CH₃,
a C₃₋₈cycloalkyl ring substituted with one or more substituents each independently selected from the group consisting of C₁₋₆alkyl, phenyl, C₂₋₆alkynyl and C₃₋₆cycloalkyl,
a C₃₋₈cycloalkyl ring containing a heteroatom and being substituted with one or more substituents each independently selected from the group consisting of C₁₋₆alkyl, CN, phenyl, C₂₋₆alkynyl and C₃₋₆cycloalkyl, said heteroatom being an oxygen atom,
a C₅₋₁₂-spirocycloalkyl optionally substituted with CH₃, and
a C₅₋₆ bridged bicyclic saturated ring system;
R⁵ is selected from the group consisting of hydrogen, C₁₋₆alkyl, C₂₋₃alkenyl, Cycle2 and Aryl3;
wherein C₁₋₆alkyl is optionally substituted with one or more substituents each independently selected from the group consisting of phenyl, methoxyphenyl, OC₁₋₆alkyl, NHSO₂CH₃, C₃₋₆cycloalkyl, and
C₃₋₆cycloalkyl containing a heteroatom, said heteroatom being an oxygen atom;
wherein Cycle2 is selected from the group consisting of
C₃₋₆cycloalkyl,
C₃₋₆cycloalkyl containing SO₂ or a heteroatom, the heteroatom being selected from the group consisting of oxygen and nitrogen,
C₃₋₆cycloalkyl substituted with CO₂R^{20a}, CONHR^{20b} or SO₂C₁₋₆alkyl,
C₃₋₆cycloalkyl containing SO₂ or a heteroatom and being substituted with CO₂R^{20a}, CONHR^{20b} or SO₂C₁₋₆alkyl, the heteroatom being selected from the group consisting of oxygen and nitrogen,
a 5-membered bridged bicyclic saturated ring substituted with CO₂C₁₋₆alkyl or CONHR^{20b}, cubanyl optionally substituted with CO₂C₁₋₆alkyl or CONHR^{20b},
isoindoline-1-one, and
indoline-2-one;
wherein R^{20a} is hydrogen or C₁₋₆alkyl;
wherein R^{20b} is C₁₋₆alkyl or C₃₋₆cycloalkyl;
wherein Aryl3 is selected from the group consisting of phenyl, monocyclic heteroaryl, and bicyclic heteroaryl, said Aryl3 being optionally substituted with one or more substituents each independently selected from the group consisting of halogen, C₁₋₆alkyl, CF₃, CF₂H, CH₂F, CN, OC₁₋₆alkyl, OCF₃, OCF₂H, OCH₂F, OC₃₋₆cycloalkyl, SO₂R²¹, SO₂NHR²², CO₂R²³, COR²⁴, CONR²⁵R²⁶, NHR²⁷, NHCOR²⁸, Cycle3 and Aryl4;
wherein R²¹ is C₁₋₆alkyl or C₃₋₆cycloalkyl;
wherein R²² is C₁₋₆alkyl or pyridine;
wherein R²³ is hydrogen or C₁₋₆alkyl;
wherein R²⁴ is selected from the group consisting of C₁₋₆alkyl, C₅₋₆heterocycle in particular C₅₋₆heterocycloalkyl and C₅₋₆heterocycle, in particular C₅₋₆heterocycloalkyl, substituted with CH₃;
wherein R²⁵ is hydrogen or CH₃;
wherein R²⁶ is selected from the group consisting of
hydrogen,
C₁₋₆alkyl,
C₁₋₆alkyl optionally substituted with one or more substituents each independently selected from the group consisting of OH, OCH₃, NH₂, CO₂H, C₃₋₆heterocycloalkyl and C₃₋₆heterocycloalkyl substituted with CH₃,
C₃₋₆cycloalkyl;
C₃₋₆cycloalkyl containing a heteroatom, said heteroatom being an oxygen atom;
C₃₋₆cycloalkyl substituted with CO₂H; and
C₃₋₆cycloalkyl containing a heteroatom and being substituted with CO₂H, said heteroatom being an oxygen atom;
wherein R²⁷ is selected from the group consisting of
C₁₋₆alkyl,
C₁₋₆alkyl substituted with C₃₋₆heterocycloalkyl, and
C₃₋₆heterocycloalkyl;
wherein R²⁸ is C₁₋₆alkyl or C₃₋₆cycloalkyl;
wherein Cycle3 is selected from the group consisting of
C₃₋₆cycloalkyl,
C₃₋₆heterocycloalkyl,
C₃₋₆heterocycloalkyl substituted with one or more substituents selected from the group consisting of OH, CH₂OH, CO₂R²⁹, NHCH₃ or NHCO₂t-Bu; and
imidazolidin-4-one substituted with CH₃;
wherein R²⁹ is hydrogen or C₁₋₆alkyl;
wherein Aryl4 is selected from the group consisting of monocyclic heteroaryl and bicyclic heteroaryl, said monocyclic or bicyclic heteroaryl being optionally substituted with one or two substituents selected from the group consisting of halogens, CF₃, CH₂F, C₁₋₆alkyl, C₃₋₆cycloalkyl, OCF₃, OCH₂F, OC₁₋₆alkyl, OC₃₋₆cycloalkyl, CO₂R³⁰, SO₂CH₃, and morpholine;
wherein R³⁰ is hydrogen or C₁₋₆alkyl;
wherein R′, R″ and R⁵ are not all hydrogen; and
wherein R⁶ is hydrogen, CH₃, CF₃ or CF₂H;
or a pharmaceutically acceptable salt thereof.
